(19) United States
(12) Patent Application Publication
(10) Pub. No.: US 2023/0295617 A1
(43) Pub. Date: Sep. 21, 2023

(54) METHODS AND COMPOSITIONS OF BIOLOGICALLY ACTIVE AGENTS

(71) Applicant: WAVE LIFE SCIENCES LTD., Singapore (SG)

(72) Inventors: Chandra Vargeese, Schwenksville, PA (US); Jason Jingxin Zhang, Walpole, MA (US); Sethumadhavan Divakaramenon, Lexington, MA (US); David Charles Donnell Butler, Medford, MA (US); Genliang Lu, Winchester, MA (US); Naoki Iwamoto, Boston, MA (US); Hailin Yang, West Roxbury, MA (US); Maria David Frank-Kamenetsky, Brookline, MA (US); Subramanian Marappan, Acton, MA (US)

(21) Appl. No.: 18/072,296

(22) Filed: Nov. 30, 2022

Related U.S. Application Data
(63) Continuation of application No. 16/098,658, filed on Nov. 2, 2018, now abandoned.
(60) Provisional application No. 62/331,961, filed on May 4, 2016.
(60) Provisional application No. 62/405,810, filed on Oct. 7, 2016.

(51) Int. Cl. C12N 15/11; A61K 47/54; A61K 31/7088

(52) U.S. Cl. CPC C12N 15/111; A61K 47/542; A61K 31/7088; C12N 2310/141; C12N 2310/14; C12N 2310/20; C12N 2320/32; C12N 2310/11; C12N 2310/16; C12N 2310/315; C12N 2310/3515

(57) ABSTRACT

In some embodiments, the present disclosure pertains to compositions and methods related to delivery of a biologically active agent, wherein the compositions comprise a biologically active agent and a lipid. In various embodiments, the lipid is selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, a composition and method are useful for delivery of a biologically active agent to a particular cell or tissue, e.g., a muscle cell or tissue.

### CROSS-REFERENCE TO RELATED APPLICATIONS

This application is a continuation of U.S. application Ser. No. 16/098,658, filed Nov. 2, 2018, which is the National Stage of International Application No. PCT/US2017/030777, filed May 3, 2017, which claims priority to United States Provisional Application Nos. 62/331,961, filed May 4, 2016, and 62/405,810, filed Oct. 7, 2016, the entirety of each of which is incorporated herein by reference.

### SEQUENCE LISTING

The instant application contains a Sequence Listing which has been submitted electronically in XML format and is hereby incorporated by reference in its entirety. Said XML copy, created on May 9, 2023, is named 2010581-1183.xml and is 3,771,428 bytes in size.

### BACKGROUND

Many biologically active agents cannot be effectively delivered to their target locations, e.g., cells, tissues, organs, etc., thereby limiting their use as therapeutics. There is a long-felt need in the art for efficient and/or effective delivery of biologically active agents to such target locations. There is a particular long-felt need in the art for efficient and/or effective delivery of biologically active agents into cells (i.e., to intracellular sites).

### SUMMARY

Among other things, the present disclosure encompasses the recognition that lipids can surprisingly enable and/or promote delivery of biologically active agents to their target location(s) (e.g., cells, tissues, organs, etc.) In some embodiments, lipids can be utilized to effectively improve delivery of biologically active agents to their target location(s) in a subject, e.g., in a mammal or human subject, etc. The present disclosure particularly documents the surprising achievement of efficient and/or effective delivery of biologically active agent(s) into cells (i.e., to intracellular location(s)). In some embodiments, the present disclosure also demonstrates surprising achievements that lipids can improve many properties, e.g., pharmacokinetics (e.g., half-life), activities, immunogenicity, etc. of biologically active agents. For example, in some embodiments, the present disclosure demonstrates that lipids can be utilized to effectively improve immune characteristics of biologically active agents, e.g., by modulating immune responses mediated by TLR9.

In light of the findings provided herein, those skilled in the art will appreciate that use of lipids can permit or facilitate delivery of biologically active agents, particularly to intracellular locations. Furthermore, in light of the findings provided herein, those skilled in the art will appreciate that use of lipids as described herein may permit or facilitate delivery of an effective and/or desired amount of biologically active agent to its target location(s) so that, for example, a comparable or higher level of the biologically active agent is achieved at the target location(s) than is observed when the biologically active agent is administered absent the lipid, in some embodiments, even though a lower amount of the biologically active agent may be administered with the lipid than without. Alternatively or additionally, in light of the findings provided herein, those skilled in the art will appreciate that use of lipids as described herein may permit or facilitate improved distribution (i.e., increased relative level of biologically active agent at a target location(s) as compared with at a non-target location(s)) relative to an appropriate control (e.g., that level observed when the biologically active agent, e.g., oligonucleotide, is comparably administered absent the lipid). Furthermore, in light of the findings provided herein, those skilled in the art will appreciate that use of lipids as described herein may permit or facilitate improved efficacy and/or low toxicities relative to an relative control (e.g., absent the lipids), for example, in some embodiments, improved properties (e.g., activities, pharmacokinetics, etc.) may permit a lower unit doses and/or less frequent administrations; in some embodiments, improved properties and/or lower toxicities (e.g., improved pharmacokinetics, undesired immune responses mediated by TLR9) may permit, if desired, higher unit doses and/or more frequent administrations. Still further, in light of the findings provided herein, those skilled in the art will appreciate that use of lipids as described herein may render biologically active agents that have otherwise been considered unsuitable for therapeutic use to be successfully used for treating various diseases, disorders and/or conditions.

In some embodiments, the present disclosure encompasses certain surprising findings, including that certain lipids are particularly effective at delivering biologically active agents to particular types of cells and tissues, including, but not limited to, cells and tissues outside the liver (e.g., extra-hepatic), including, but not limited to, muscle cells and tissues. In some embodiments, the present disclosure provides technologies (compounds, compositions, methods, etc.) that are surprisingly effective at delivering biologically active agents to muscle cells and tissues, e.g., of heart, thoracic diaphragm, skeletal muscle cells and tissues, gastrocnemius, quadriceps, triceps, and/or smooth muscle cells and tissues, etc.

In some embodiments, the present disclosure provides a composition comprising a biologically active agent and a lipid. Many lipids can be utilized in provided technologies in accordance with the present disclosure. In some embodiments, a lipid comprises an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein. In some embodiments, a lipid comprises an optionally substituted C₁₀-C₈₀ saturated or partially unsaturated, aliphatic chain. In some embodiments, a lipid comprises an optionally substituted C₁₀-C₈₀ linear, saturated or partially unsaturated, aliphatic chain. In some embodiments, a lipid comprises a C₁₀-C₈₀ linear, saturated or partially unsaturated, aliphatic chain, optionally substituted with one or more C₁₋₄ aliphatic group. In some embodiments, a lipid comprises an unsubstituted C₁₀-C₈₀ linear, saturated or partially unsaturated, aliphatic chain. In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain. In some embodiments, a lipid comprises an optionally substituted C₁₀-C₈₀ saturated or partially unsaturated, aliphatic chain. In some embodiments, a lipid comprises an optionally substituted C₁₀-C₈₀ linear, saturated or partially unsaturated, aliphatic chain. In some embodiments, a lipid comprises a C₁₀-C₈₀ linear, saturated or partially unsaturated, aliphatic chain, optionally substituted with one or more C₁₋₄ aliphatic group. In some embodiments, a lipid comprises an unsubstituted C₁₀-C₈₀ linear, saturated or partially unsaturated, aliphatic chain. In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain. In some embodiments, a lipid comprises an optionally substituted C₁₀-C₄₀ saturated or partially unsaturated, aliphatic chain. In some embodiments, a lipid comprises an optionally substituted C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, a lipid comprises an unsubstituted C₁₀-C₄₀ linear, saturated or partially unsaturated, aliphatic chain. In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₄₀ linear, saturated or partially unsaturated, aliphatic chain.

In some embodiments, the present disclosure provides a composition comprising a biologically active agent and a lipid. Many lipids can be utilized in provided technologies in accordance with the present disclosure. In some embodiments, a lipid comprises an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein. In some embodiments, a lipid comprises an optionally substituted C₁₀-C₈₀ saturated or partially unsaturated, aliphatic chain. In some embodiments, a lipid comprises an optionally substituted C₁₀-C₈₀ linear, saturated or partially unsaturated, aliphatic chain. In some embodiments, a lipid comprises a C₁₀-C₈₀ linear, saturated or partially unsaturated, aliphatic chain, optionally substituted with one or more C₁₋₄ aliphatic group. In some embodiments, a lipid comprises an unsubstituted C₁₀-C₈₀ linear, saturated or partially unsaturated, aliphatic chain. In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain. In some embodiments, a lipid comprises an optionally substituted C₁₀-C₆₀ saturated or partially unsaturated, aliphatic chain. In some embodiments, a lipid comprises an optionally substituted C₁₀-C₆₀ linear, saturated or partially unsaturated, aliphatic chain. In some embodiments, a lipid comprises a C₁₀-C₆₀ linear, saturated or partially unsaturated, aliphatic chain, optionally substituted with one or more C₁₋₄ aliphatic group. In some embodiments, a lipid comprises an unsubstituted C₁₀-C₆₀ linear, saturated or partially unsaturated, aliphatic chain. In some embodiments, a lipid comprises no more than one optionally substituted C₁₀-C₆₀ linear, saturated or partially unsaturated, aliphatic chain. In some embodiments, a lipid comprises two or more optionally substituted C₁₀-C₆₀ linear, saturated or partially unsaturated, aliphatic chain. In some embodiments, a lipid comprises an optionally substituted C₁₀-C₄₀ saturated or partially unsaturated, aliphatic chain. In some embodiments, a lipid comprises an optionally substituted C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, a lipid comprises an unsubstituted C₁₀-C₄₀ linear, saturated or partially unsaturated, aliphatic chain. In some embodiments, a lipid comprises no more than one optionally substituted C₁₀-C₆₀ linear, saturated or partially unsaturated, aliphatic chain. In some embodiments, a lipid comprises two or more optionally substituted C₁₀-C₄₀ linear, saturated or partially unsaturated, aliphatic chain.

In some embodiments, the present disclosure provides a composition comprising a biologically active agent and a lipid. Many lipids can be utilized in provided technologies in accordance with the present disclosure. In some embodiments, a lipid comprises an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein. In some embodiments, a lipid comprises an optionally substituted C₁₀-C₈₀ saturated or partially unsaturated, aliphatic chain. In some embodiments, a lipid comprises an optionally substituted C₁₀-C₈₀ linear, saturated or partially unsaturated, aliphatic chain. In some embodiments, a lipid comprises a C₁₀-C₈₀ linear, saturated or partially unsaturated, aliphatic chain, optionally substituted with one or more C₁₋₄ aliphatic group. In some embodiments, a lipid comprises an unsubstituted C₁₀-C₈₀ linear, saturated or partially unsaturated, aliphatic chain. In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain. In some embodiments, a lipid comprises an optionally substituted C₁₀-C₄₀ saturated or partially unsaturated, aliphatic chain. In some embodiments, a lipid comprises an optionally substituted C₁₀-C₆₀ linear, saturated or partially unsaturated, aliphatic chain. In some embodiments, a lipid comprises a C₁₀-C₆₀ linear, saturated or partially unsaturated, aliphatic chain, optionally substituted with one or more C₁₋₄ aliphatic group. In some embodiments, a lipid comprises an unsubstituted C₁₀-C₆₀ linear, saturated or partially unsaturated, aliphatic chain. In some embodiments, a lipid comprises no more than one optionally substituted C₁₀-C₆₀ linear, saturated or partially unsaturated, aliphatic chain. In some embodiments, a lipid comprises two or more optionally substituted C₁₀-C₆₀ linear, saturated or partially unsaturated, aliphatic chain. In some embodiments, a lipid comprises an optionally substituted C₁₀-C₆₀ saturated or partially unsaturated, aliphatic chain. In some embodiments, a lipid comprises an optionally substituted C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, a lipid comprises an unsubstituted C₁₀-C₄₀ linear, saturated or partially unsaturated, aliphatic chain. In some embodiments, a lipid comprises no more than one optionally substituted C₁₀-C₄₀ linear, saturated or partially unsaturated, aliphatic chain. In some embodiments, a lipid comprises two or more optionally substituted C₁₀-C₄₀ linear, saturated or partially unsaturated, aliphatic chain.

In some embodiments, the present disclosure provides a composition comprising a biologically active agent and a lipid comprising a C₁₀-C₈₀ linear, saturated or partially unsaturated, aliphatic chain. In some embodiments, the present disclosure pertains to a composition comprising a biologically active agent and a lipid comprising a C₁₀-C₈₀ linear, saturated or partially unsaturated, aliphatic chain, optionally substituted with one or more C₁₋₄ aliphatic group. In some embodiments, the present disclosure provides a composition comprising a biologically active agent and a lipid comprising a C₁₀-C₆₀ linear, saturated or partially unsaturated, aliphatic chain. In some embodiments, the present disclosure pertains to a composition comprising a biologically active agent and a lipid comprising a C₁₀-C₆₀ linear, saturated or partially unsaturated, aliphatic chain, optionally substituted with one or more C₁₋₄ aliphatic group. In some embodiments, the present disclosure provides a composition comprising a biologically active agent and a lipid comprising a C₁₀-C₄₀ linear, saturated or partially unsaturated, aliphatic chain. In some embodiments, the present disclosure pertains to a composition comprising a biologically active agent and a lipid comprising a C₁₀-C₄₀ linear, saturated or partially unsaturated, aliphatic chain, optionally substituted with one or more C₁₋₄ aliphatic group.

In some embodiments, the present disclosure pertains to a composition comprising a biologically active agent and a lipid selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, a lipid has a structure of any of:

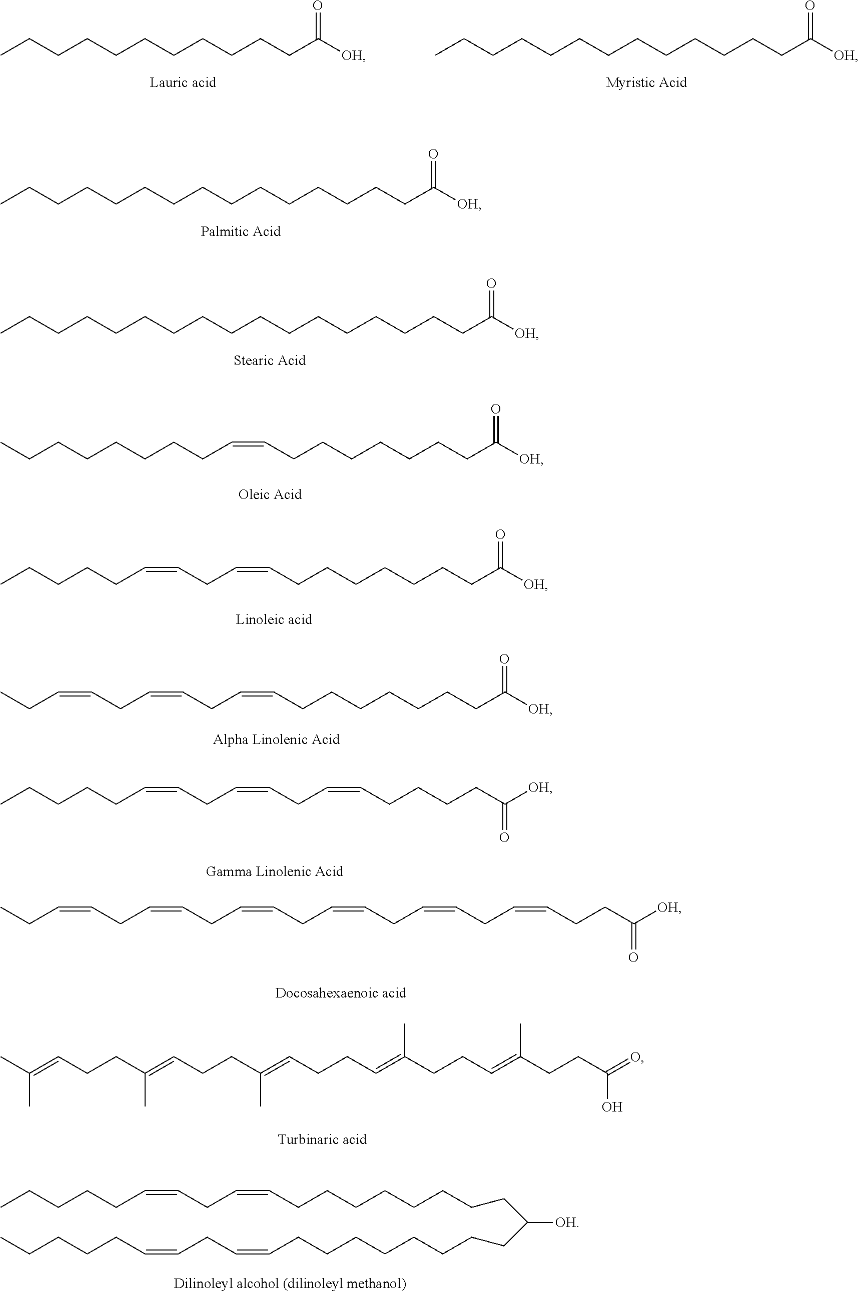

In some embodiments, a lipid is conjugated to a biologically active agent. A person having ordinary skill in the art appreciates that various technologies can be utilized to conjugate lipids to biologically active agent in accordance with the present disclosure. In some embodiments, a lipid is not conjugated to a biologically active agent.

Various biologically active agents can be effectively delivered to their targets in accordance with the present disclosure. In some embodiments, a biologically active agent is selected from the group consisting of: a small molecule, a peptide, a protein, a component of a CRISPR-Cas system, a carbohydrate, a therapeutic agent, a chemotherapeutic agent, a vaccine, a nucleic acid, and a lipid. In some embodiments, a nucleic acid comprises one or more: nucleotides (e.g., natural nucleotides, modified nucleotides nucleotide analogs, etc.). In some embodiments, a nucleic acid is an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, a vector, or a portion thereof. In some embodiments, a biologically active agent is an oligonucleotide. In some embodiments, the present disclosure provides compositions comprising an oligonucleotide and a lipid. Among other things, such compositions are surprisingly effective at delivering oligonucleotides to their target locations, in some embodiments, delivering oligonucleotides into the cells at the target locations. In some embodiments, provided technologies are surprisingly effective at delivering oligonucleotides to muscle cells, tissues, etc. In some embodiments, provided compounds, for example, oligonucleotides conjugated with lipids, have unexpectedly improved properties, e.g., improved activities, improved pharmacokinetics, lowered toxicities (e.g., lowered undesired immuno responses), improved delivery to targets (e.g., cells, tissues, organs, organisms, etc.), etc. In some embodiments, an oligonucleotide is an oligonucleotide described in Patent Application Publications US20120316224, US20140194610, US20150211006, and WO2015107425, and U.S. Pat. Nos. 9,243,245; 9,249,416; 9,175,286; 9,234,198; 8,895,309; 8,741,863; 8,097,596; 5,854,223; 5,756,476; and 8,871,918; the oligonucleotides and oligonucleotide compositions of each of which are incorporated herein by reference. In some embodiments, an oligonucleotide comprises one or more chiral internucleotidic linkages. In some embodiments, for oligonucleotides comprising one or more chiral internucleotidic linkages, a provided composition is a stereorandom composition of such oligonucleotides in that stereochemistry of each of the chiral internucleotidic linkages is not controlled. In some embodiments, a stereorandom composition is prepared by oligonucleotide synthesis without dedicated efforts e.g., through chiral auxiliaries, etc. to control the stereochemistry of each chiral internucleotidic linkages. In some embodiments, for oligonucleotides comprising one or more chiral internucleotidic linkages, a provided composition is a chirally controlled oligonucleotide composition of such oligonucleotides in that stereochemistry of at least one of the chiral internucleotidic linkages is controlled. In some embodiments, stereochemistry of each of the chiral internucleotidic linkages is independently controlled, and a provided composition is a completely chirally controlled oligonucleotide composition. In some embodiments, stereochemistry of one or more chiral internucleotidic linkages is controlled (chiral controlled internucleotidic linkages) while stereochemistry of one or more chiral internucleotidic linkages is not controlled (stereorandom/non-chirally controlled internucleotidic linkages), and a provided composition is a partially chirally controlled oligonucleotide composition. In some embodiments, a chirally controlled oligonucleotide composition can be prepared by oligonucleotide synthesis comprising stereoselective formation of one or more or all chiral internucleotidic linkages using, for example, technologies described in Patent Application Publications US20120316224, US20140194610, US20150211006, and WO2015107425, the technologies of each of which are incorporated herein by reference. In some embodiments, a provided composition comprises a chirally controlled oligonucleotide composition described in Patent Application Publications US20120316224, US20140194610, US20150211006, and WO2015107425, the chirally controlled oligonucleotide compositions of each of which are incorporated herein by reference, and a lipid. In some embodiments, a lipid is conjugated to oligonucleotides comprising stereochemically controlled internucleotidic linkages.

In some embodiments, the present disclosure provides a chirally controlled oligonucleotide composition comprising a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages, and level of the plurality of oligonucleotides in the composition is pre-determined;

one or more oligonucleotides of the plurality are independently conjugated to a lipid; and one or more oligonucleotides of the plurality are optionally and independently conjugated to a target component.

In some embodiments, the present disclosure provides a chirally controlled oligonucleotide composition comprising a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages, and at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% of oligonucleotides in the composition that share the common base sequence, the common pattern of backbone linkages; and the common pattern of backbone phosphorus modifications share the same stereochemistry at the one or more chiral internucleotidic linkages;

one or more oligonucleotides of the plurality are independently conjugated to a lipid; and one or more oligonucleotides of the plurality are optionally and independently conjugated to a target component.

In some embodiments, the present disclosure provides an oligonucleotide composition comprising a plurality of oligonucleotides having the structure of:

A^{c}-[-L^{LD}-(R^{LD})ₐ]_{b}, or [(A^{c})ₐ-L^{LD}]_{b}-R^{LD},

wherein:
A^{c} is a biologically active agent;
a is 1-1000;
b is 1-1000;
each L^{LD} is independently a linker moiety or a covalent bond; and
each R^{LD} is independently a lipid moiety or a targeting component.

In some embodiments, the present disclosure provides an oligonucleotide composition comprising a plurality of oligonucleotides having the structure of:

A^{c}-[-L^{LD}-(R^{LD})ₐ]_{b}, or [(A^{c})ₐ-L^{LD}]_{b}-R^{LD},

wherein:
A^{c} is a biologically active agent;
a is 1-1000;
b is 1-1000;
each L^{LD} is independently a linker moiety; and
each R^{LD} is independently a lipid moiety or a targeting component, wherein at least one R^{LD} is a lipid moiety.

In some embodiments, the present disclosure provides an oligonucleotide composition comprising a plurality of oligonucleotides having the structure of:

A^{c}-[-L^{LD}-(R^{LD})ₐ]_{b}, or [(A^{c})ₐ-L^{LD}]_{b}-R^{LD},

wherein:
A^{c} is a biologically active agent;
a is 1-1000;
b is 1-1000;
each L^{LD} is independently a covalent bond or an optionally substituted, C₁-C₈₀ saturated or partially unsaturated aliphatic group, wherein one or more methylene units are optionally and independently replaced by T^{LD} or an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
each R^{LD} is independently an optionally substituted, C₁-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
T^{LD} has the structure of:

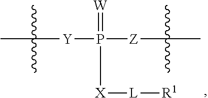

W is O, S or Se;
each of X, Y and Z is independently —O—, —S—, —N(-L-R′)—, or L;
L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene; and
each R is independently hydrogen, or an optionally substituted group selected from C₁-C₆ aliphatic, carbocyclyl, aryl, heteroaryl, and heterocyclyl.

In some embodiments, A^{c} is an oligonucleotide chain ([H]_{b}-A^{c} is an oligonucleotide). In some embodiments, [H]_{b}-A^{c} is an oligonucleotide of any of the Tables. In some embodiments, H-A^{c} is a small molecule. In some embodiments, H-A^{c} is a peptide. In some embodiments, H-A^{c} is a protein.

In some embodiments, P in T^{LD} is P*. In some embodiments, a conjugate has the structure of [(A^{c})ₐ-L^{LD}]_{b}-R^{LD}. In some embodiments, a conjugate has the structure of (A^{c})ₐ-L^{LD}-R^{LD}. In some embodiments, a is 1-100. In some embodiments, a is 1-50. In some embodiments, a is 1-40. In some embodiments, a is 1-30. In some embodiments, a is 1-20. In some embodiments, a is 1-15. In some embodiments, a is 1-10. In some embodiments, a is 1-9. In some embodiments, a is 1-8. In some embodiments, a is 1-7. In some embodiments, a is 1-6. In some embodiments, a is 1-5. In some embodiments, a is 1-4. In some embodiments, a is 1-3. In some embodiments, a is 1-2. In some embodiments, a is 1. In some embodiments, a is 2. In some embodiments, a is 3. In some embodiments, a is 4. In some embodiments, a is 5. In some embodiments, a is 6. In some embodiments, a is 7. In some embodiments, a is 8. In some embodiments, a is 9. In some embodiments, a is 10. In some embodiments, a is more than 10. In some embodiments, b is 1-100. In some embodiments, b is 1-50. In some embodiments, b is 1-40. In some embodiments, b is 1-30. In some embodiments, b is 1-20. In some embodiments, b is 1-15. In some embodiments, b is 1-10. In some embodiments, b is 1-9. In some embodiments, b is 1-8. In some embodiments, b is 1-7. In some embodiments, b is 1-6. In some embodiments, b is 1-5. In some embodiments, b is 1-4. In some embodiments, b is 1-3. In some embodiments, b is 1-2. In some embodiments, b is 1. In some embodiments, b is 2. In some embodiments, b is 3. In some embodiments, b is 4. In some embodiments, b is 5. In some embodiments, b is 6. In some embodiments, b is 7. In some embodiments, b is 8. In some embodiments, b is 9. In some embodiments, b is 10. In some embodiments, b is more than 10. In some embodiments, a conjugate has the structure of A^{c}-L^{LD}-R^{LD}. In some embodiments, A^{c} is conjugated through one or more of its sugar, base and/or internucleotidic linkage moieties. In some embodiments, A^{c} is conjugated through its 5′-OH (5′-O—). In some embodiments, A^{c} is conjugated through its 3′-OH (3′-O—). In some embodiments, before conjugation, A^{c}-(H)_{b} (b is an integer of 1-1000 depending on valency of A^{c}) is an oligonucleotide as described herein, for example, one of those described in any one of the Tables. In some embodiments, L^{LD} is -L-. In some embodiments, L^{LD} comprises a phosphorothioate group. In some embodiments, L^{LD} is —C(O)NH—(CH₂)₆—OP(═O)(S⁻)—O—. In some embodiments, the —C(O)NH end is connected to R^{LD}, and the —O— end is connected to the oligonucleotide, e.g., through 5′- or 3′-end. In some embodiments, R^{LD} is optionally substituted C₁₀, C₁₅, C₁₆, C₁₇, C₁₈, C₁₉, C₂₀, C₂₁, C₂₂, C₂₃, C₂₄, or C₂₅ to C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₆₀, C₇₀, or C₈₀ aliphatic. In some embodiments, R^{LD} is optionally substituted C₁₀₋₈₀ aliphatic. In some embodiments, R^{LD} is optionally substituted C₂₀₋₈₀ aliphatic. In some embodiments, R^{LD} is optionally substituted C₁₀₋₇₀ aliphatic. In some embodiments, R^{LD} is optionally substituted C₂₀₋₇₀ aliphatic. In some embodiments, R^{LD} is optionally substituted C₁₀₋₆₀ aliphatic. In some embodiments, R^{LD} is optionally substituted C₂₀₋₆₀ aliphatic. In some embodiments, R^{LD} is optionally substituted C₁₀₋₅₀ aliphatic. In some embodiments, R^{LD} is optionally substituted C₂₀₋₅₀ aliphatic. In some embodiments, R^{LD} is optionally substituted C₁₀₋₄₀ aliphatic. In some embodiments, R^{LD} is optionally substituted C₂₀₋₄₀ aliphatic. In some embodiments, R^{LD} is optionally substituted C₁₀₋₃₀ aliphatic. In some embodiments, R^{LD} is optionally substituted C₂₀₋₃₀ aliphatic. In some embodiments, R^{LD} is unsubstituted C₁₀, C₁₅, C₁₆, C₁₇, C₁₈, C₁₉, C₂₀, C₂₁, C₂₂, C₂₃, C₂₄, or C₂₅ to C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₆₀, C₇₀, or C₈₀ aliphatic. In some embodiments, R^{LD} is unsubstituted C₁₀₋₈₀ aliphatic. In some embodiments, R^{LD} is unsubstituted C₂₀₋₈₀ aliphatic. In some embodiments, R^{LD} is unsubstituted C₁₀₋₇₀ aliphatic. In some embodiments, R^{LD} is unsubstituted C₂₀₋₇₀ aliphatic. In some embodiments, R^{LD} is unsubstituted C₁₀₋₆₀ aliphatic. In some embodiments, R^{LD} is unsubstituted C₂₀₋₆₀ aliphatic. In some embodiments, R^{LD} is unsubstituted C₁₀₋₅₀ aliphatic. In some embodiments, R^{LD} is unsubstituted C₂₀₋₅₀ aliphatic. In some embodiments, R^{LD} is unsubstituted C₁₀₋₄₀ aliphatic. In some embodiments, R^{LD} is unsubstituted C₂₀₋₄₀ aliphatic. In some embodiments, R^{LD} is unsubstituted C₁₀₋₃₀ aliphatic. In some embodiments, R^{LD} is unsubstituted C₂₀₋₃₀ aliphatic.

In some embodiments, a plurality of oligonucleotides share the same stereochemistry at one or more chiral internucleotidic linkages (chirally controlled internucleotidic linkages). In some embodiments, they share the same stereochemistry at two or more chiral internucleotidic linkages. In some embodiments, they share the same stereochemistry at three or more chiral internucleotidic linkages. In some embodiments, they share the same stereochemistry at four or more chiral internucleotidic linkages. In some embodiments, they share the same stereochemistry at five or more chiral internucleotidic linkages. In some embodiments, they share the same stereochemistry at six or more chiral internucleotidic linkages. In some embodiments, they share the same stereochemistry at seven or more chiral internucleotidic linkages. In some embodiments, they share the same stereochemistry at eight or more chiral internucleotidic linkages. In some embodiments, they share the same stereochemistry at nine or more chiral internucleotidic linkages. In some embodiments, they share the same stereochemistry at ten or more chiral internucleotidic linkages. In some embodiments, they share the same stereochemistry at 11 or more chiral internucleotidic linkages. In some embodiments, they share the same stereochemistry at 12 or more chiral internucleotidic linkages. In some embodiments, they share the same stereochemistry at 13 or more chiral internucleotidic linkages. In some embodiments, they share the same stereochemistry at 14 or more chiral internucleotidic linkages. In some embodiments, they share the same stereochemistry at 15 or more chiral internucleotidic linkages. In some embodiments, they share the same stereochemistry at 10% or more of the chiral internucleotidic linkages. In some embodiments, they share the same stereochemistry at 20% or more of the chiral internucleotidic linkages. In some embodiments, they share the same stereochemistry at 30% or more of the chiral internucleotidic linkages. In some embodiments, they share the same stereochemistry at 40% or more of the chiral internucleotidic linkages. In some embodiments, they share the same stereochemistry at 50% or more of the chiral internucleotidic linkages. In some embodiments, they share the same stereochemistry at 60% or more of the chiral internucleotidic linkages. In some embodiments, they share the same stereochemistry at 70% or more of the chiral internucleotidic linkages. In some embodiments, they share the same stereochemistry at 80% or more of the chiral internucleotidic linkages. In some embodiments, they share the same stereochemistry at 90% or more of the chiral internucleotidic linkages. In some embodiments, they share the same stereochemistry at 95% or more of the chiral internucleotidic linkages. In some embodiments, they share the same stereochemistry at 96% or more of the chiral internucleotidic linkages. In some embodiments, they share the same stereochemistry at 97% or more of the chiral internucleotidic linkages. In some embodiments, they share the same stereochemistry at 98% or more of the chiral internucleotidic linkages. In some embodiments, they share the same stereochemistry at each of the chiral internucleotidic linkages. As readily appreciated by a person having ordinary skill in the art and illustrated the examples, chiral internucleotidic linkages where a plurality of oligonucleotides share the same stereochemistry can independently be either Rp or Sp, e.g., at a first chiral internucleotidic linkage a plurality of oligonucleotides are all Rp while at a second position they are all Sp (RpSp; can also be RpRp, SpSp, or SpRp as desired).

In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% of oligonucleotides in a provided composition that share the common base sequence, the common pattern of backbone linkages; and the common pattern of backbone phosphorus modifications share the same stereochemistry at the one or more chiral internucleotidic linkages. In some embodiments, the percentage is at least 0.5%. In some embodiments, the percentage is at least 1%. In some embodiments, the percentage is at least 2%. In some embodiments, the percentage is at least 3%. In some embodiments, the percentage is at least 4%. In some embodiments, the percentage is at least 5%. In some embodiments, the percentage is at least 6%. In some embodiments, the percentage is at least 7%. In some embodiments, the percentage is at least 8%. In some embodiments, the percentage is at least 9%. In some embodiments, the percentage is at least 10%. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 70%. In some embodiments, the percentage is at least 75%. In some embodiments, the percentage is at least 80%. In some embodiments, the percentage is at least 81%. In some embodiments, the percentage is at least 82%. In some embodiments, the percentage is at least 83%. In some embodiments, the percentage is at least 84%. In some embodiments, the percentage is at least 85%. In some embodiments, the percentage is at least 86%. In some embodiments, the percentage is at least 87%. In some embodiments, the percentage is at least 88%. In some embodiments, the percentage is at least 89%. In some embodiments, the percentage is at least 90%. In some embodiments, the percentage is at least 91%. In some embodiments, the percentage is at least 92%. In some embodiments, the percentage is at least 93%. In some embodiments, the percentage is at least 94%. In some embodiments, the percentage is at least 95%. In some embodiments, the percentage is at least 96%. In some embodiments, the percentage is at least 97%. In some embodiments, the percentage is at least 98%. In some embodiments, the percentage is at least 99%.

In some embodiments, oligonucleotides that share the common base sequence, the common pattern of backbone linkages, the common pattern of backbone phosphorus modifications and the same stereochemistry at the one or more chiral internucleotidic linkages are enriched, for example, relative to oligonucleotides that share the common base sequence, the common pattern of backbone linkages, the common pattern of backbone phosphorus modifications but not the same stereochemistry at the one or more chiral internucleotidic linkages. In some embodiments, as understood by a person having ordinary skill in the art, the enrichment is from the use of one or more provided technologies that enable stereoselective (chirally controlled) formation of each of the internucleotidic linkages where the oligonucleotides share the same stereochemistry.

In some embodiments, oligonucleotides that share the common base sequence, the common pattern of backbone linkages, the common pattern of backbone phosphorus modifications and the same stereochemistry at the one or more chiral internucleotidic linkages are enriched at least 5 fold (such oligonucleotides have a fraction of 5*(½ⁿ) of oligonucleotides that share the common base sequence, the common pattern of backbone linkages, and the common pattern of backbone phosphorus modifications, wherein n is the number of internucleotidic linkages where such oligonucleotides share the same stereochemistry; or oligonucleotides that share the common base sequence, the common pattern of backbone linkages, the common pattern of backbone phosphorus modifications but not the same stereochemistry at the one or more chiral internucleotidic linkages are no more than [1-(1/2ⁿ)]/5 of oligonucleotides that share the common base sequence, the common pattern of backbone linkages, and the common pattern of backbone phosphorus modifications) compared to a stereorandom preparation of the oligonucleotides wherein none of the internucleotidic linkages are chirally controlled (oligonucleotides that share the common base sequence, the common pattern of backbone linkages, the common pattern of backbone phosphorus modifications, and the same stereochemistry at the one or more chiral internucleotidic linkages are typically considered to have a fraction of ½ⁿ of oligonucleotides that share the common base sequence, the common pattern of backbone linkages, and the common pattern of backbone phosphorus modifications, wherein n is the number of chiral internucleotidic linkages wherein the oligonucleotides share the same stereochemistry, and oligonucleotides that share the common base sequence, the common pattern of backbone linkages, the common pattern of backbone phosphorus modifications but are not of the particular oligonucleotide type are typically considered to have a fraction of [1-(1/2ⁿ)] of oligonucleotides that share the common base sequence, the common pattern of backbone linkages, and the common pattern of backbone phosphorus modifications). In some embodiments, the enrichment is at least 20 fold. In some embodiments, the enrichment is at least 30 fold. In some embodiments, the enrichment is at least 40 fold. In some embodiments, the enrichment is at least 50 fold. In some embodiments, the enrichment is at least 60 fold. In some embodiments, the enrichment is at least 70 fold. In some embodiments, the enrichment is at least 80 fold. In some embodiments, the enrichment is at least 90 fold. In some embodiments, the enrichment is at least 100 fold. In some embodiments, the enrichment is at least 200 fold. In some embodiments, the enrichment is at least 300 fold. In some embodiments, the enrichment is at least 400 fold. In some embodiments, the enrichment is at least 500 fold. In some embodiments, the enrichment is at least 600 fold. In some embodiments, the enrichment is at least 700 fold. In some embodiments, the enrichment is at least 800 fold. In some embodiments, the enrichment is at least 900 fold. In some embodiments, the enrichment is at least 1,000 fold. In some embodiments, the enrichment is at least 2,000 fold. In some embodiments, the enrichment is at least 4,000 fold. In some embodiments, the enrichment is at least 8,000 fold. In some embodiments, the enrichment is at least 10,000 fold. In some embodiments, the enrichment is at least 20,000 fold. In some embodiments, the enrichment is at least (1.5)ⁿ. In some embodiments, the enrichment is at least (1.6)ⁿ. In some embodiments, the enrichment is at least (1.7)ⁿ. In some embodiments, the enrichment is at least (1.1)ⁿ. In some embodiments, the enrichment is at least (1.8)ⁿ. In some embodiments, the enrichment is at least (1.9)ⁿ. In some embodiments, the enrichment is at least 2ⁿ. In some embodiments, the enrichment is at least 3ⁿ. In some embodiments, the enrichment is at least 4ⁿ. In some embodiments, the enrichment is at least 5ⁿ. In some embodiments, the enrichment is at least 6ⁿ. In some embodiments, the enrichment is at least 7ⁿ. In some embodiments, the enrichment is at least 8ⁿ. In some embodiments, the enrichment is at least 9ⁿ. In some embodiments, the enrichment is at least 10ⁿ. In some embodiments, the enrichment is at least 15ⁿ. In some embodiments, the enrichment is at least 20ⁿ. In some embodiments, the enrichment is at least 25ⁿ. In some embodiments, the enrichment is at least 30ⁿ. In some embodiments, the enrichment is at least 40ⁿ. In some embodiments, the enrichment is at least 50ⁿ. In some embodiments, the enrichment is at least 100ⁿ. In some embodiments, enrichment is measured by increase of the fraction of oligonucleotides that share the common base sequence, the common pattern of backbone linkages, the common pattern of backbone phosphorus modifications and the same stereochemistry at the one or more chiral internucleotidic linkages. In some embodiments, an enrichment is measured by decrease of the fraction of oligonucleotides that share the common base sequence, the common pattern of backbone linkages, the common pattern of backbone phosphorus modifications but not the same stereochemistry at the one or more chiral internucleotidic linkages.

In some embodiments, oligonucleotides of a particular type in a chirally controlled oligonucleotide composition are structurally identical (including stereochemically) and are enriched at least 5 fold (oligonucleotides of the particular type have a fraction of 5*(½ⁿ) of oligonucleotides that have the base sequence, the pattern of backbone linkages, and the pattern of backbone phosphorus modifications of the particular oligonucleotide type, wherein n is the number of chiral internucleotidic linkages; or oligonucleotides that have the base sequence, the pattern of backbone linkages, and the pattern of backbone phosphorus modifications of the particular oligonucleotide type but are not of the particular oligonucleotide type are no more than [1-(1/2ⁿ)]/5 of oligonucleotides that have the base sequence, the pattern of backbone linkages, and the pattern of backbone phosphorus modifications of the particular oligonucleotide type) compared to a stereorandom preparation of the oligonucleotides (oligonucleotides of the particular type are typically considered to have a fraction of ½ⁿ of oligonucleotides that have the base sequence, the pattern of backbone linkages, and the pattern of backbone phosphorus modifications of the particular oligonucleotide type, wherein n is the number of chiral internucleotidic linkages, and oligonucleotides that have the base sequence, the pattern of backbone linkages, and the pattern of backbone phosphorus modifications of the particular oligonucleotide type but are not of the particular oligonucleotide type are typically considered to have a fraction of [1-(1/2ⁿ)] of oligonucleotides that have the base sequence, the pattern of backbone linkages, and the pattern of backbone phosphorus modifications of the particular oligonucleotide type). In some embodiments, the enrichment is at least 20 fold. In some embodiments, the enrichment is at least 30 fold. In some embodiments, the enrichment is at least 40 fold. In some embodiments, the enrichment is at least 50 fold. In some embodiments, the enrichment is at least 60 fold. In some embodiments, the enrichment is at least 70 fold. In some embodiments, the enrichment is at least 80 fold. In some embodiments, the enrichment is at least 90 fold. In some embodiments, the enrichment is at least 100 fold. In some embodiments, the enrichment is at least 200 fold. In some embodiments, the enrichment is at least 300 fold. In some embodiments, the enrichment is at least 400 fold. In some embodiments, the enrichment is at least 500 fold. In some embodiments, the enrichment is at least 600 fold. In some embodiments, the enrichment is at least 700 fold. In some embodiments, the enrichment is at least 800 fold. In some embodiments, the enrichment is at least 900 fold. In some embodiments, the enrichment is at least 1,000 fold. In some embodiments, the enrichment is at least 2,000 fold. In some embodiments, the enrichment is at least 4,000 fold. In some embodiments, the enrichment is at least 8,000 fold. In some embodiments, the enrichment is at least 10,000 fold. In some embodiments, the enrichment is at least 20,000 fold. In some embodiments, the enrichment is at least (1.5)ⁿ. In some embodiments, the enrichment is at least (1.6)ⁿ. In some embodiments, the enrichment is at least (1.7)ⁿ. In some embodiments, the enrichment is at least (1.1)ⁿ. In some embodiments, the enrichment is at least (1.8)ⁿ. In some embodiments, the enrichment is at least (1.9)ⁿ. In some embodiments, the enrichment is at least 2ⁿ. In some embodiments, the enrichment is at least 3ⁿ. In some embodiments, the enrichment is at least 4ⁿ. In some embodiments, the enrichment is at least 5ⁿ. In some embodiments, the enrichment is at least 6ⁿ. In some embodiments, the enrichment is at least 7ⁿ. In some embodiments, the enrichment is at least 8ⁿ. In some embodiments, the enrichment is at least 9ⁿ. In some embodiments, the enrichment is at least 10ⁿ. In some embodiments, the enrichment is at least 15ⁿ. In some embodiments, the enrichment is at least 20ⁿ. In some embodiments, the enrichment is at least 25ⁿ. In some embodiments, the enrichment is at least 30ⁿ. In some embodiments, the enrichment is at least 40ⁿ. In some embodiments, the enrichment is at least 50ⁿ. In some embodiments, the enrichment is at least 100ⁿ. In some embodiments, enrichment is measured by increase of the fraction of oligonucleotides of the particular oligonucleotide type in oligonucleotides that have the base sequence, the pattern of backbone linkages, and the pattern of backbone phosphorus modifications of the particular oligonucleotide type. In some embodiments, an enrichment is measured by decrease of the fraction of oligonucleotides that have the base sequence, the pattern of backbone linkages, and the pattern of backbone phosphorus modifications of the particular oligonucleotide type but are not of the particular oligonucleotide type in oligonucleotides that have the base sequence, the pattern of backbone linkages, and the pattern of backbone phosphorus modifications of the particular oligonucleotide type.

In some embodiments, a composition further comprises a targeting component. A targeting component can be either conjugated or not conjugated to a lipid or a biologically active agent. In some embodiments, a targeting component is conjugated to a biologically active agent. In some embodiments, a biologically active agent is conjugated to both a lipid and a targeting component. Various targeting components can be used in accordance with the present disclosure, e.g., lipids, antibodies, peptides, carbohydrates, etc.

In some embodiments, the present disclosure encompasses the use of a composition comprising a lipid and a biologically active agent. In some embodiments, the present disclosure provides methods for delivering a biologically active agent to a target location comprising administering a provided composition. In some embodiments, a provided method delivers a biologically active agent into a cell. In some embodiments, a provided method delivers a biologically active agent into a muscle cell. In some embodiments, a provided method delivers a biologically active agent into a cell within a tissue. In some embodiments, a provided method delivers a biologically active agent into a cell within an organ. In some embodiments, a provided method delivers a biologically active agent into a cell within a subject, comprising administering to the subject a provided composition. In some embodiments, a provided method delivers a biologically active agent into cytoplasm. In some embodiments, a provided method delivers a biologically active agent into nucleus.

In some embodiments, the present disclosure pertains to methods related to the delivery of a biologically active agent to a muscle cell or tissue, or a muscle cell or tissue in a mammal (e.g., a human subject), which method pertains to a use of a composition comprising a biologically active agent and a lipid and any one or more additional components selected from: a polynucleotide, a dye, an intercalating agent (e.g. an acridine), carbonic anhydrase inhibitor, a cross-linker (e.g. psoralene, or mitomycin C), a porphyrin (e.g., TPPC4, texaphyrin, or Sapphyrin), a polycyclic aromatic hydrocarbon (e.g., phenazine, or dihydrophenazine), an artificial endonuclease, a chelating agent, EDTA, an alkylating agent, a phosphate, an amino, a mercapto, a PEG (e.g., PEG-40K), MPEG, [MPEG]₂, a polyamino, an alkyl, a substituted alkyl, a radiolabeled marker, an enzyme, a hapten (e.g. biotin), a transport/absorption facilitator (e.g., aspirin, vitamin E, or folic acid), a synthetic ribonuclease, a protein, e.g., a glycoprotein, or peptide, e.g., a molecule having a specific affinity for a co-ligand, or antibody e.g., an antibody, a hormone, a hormone receptor, a non-peptidic species, a lipid, a lectin, a carbohydrate, a vitamin, a cofactor, or a drug. In some embodiments, the present disclosure pertains to compositions or methods related to a composition comprising a biologically active agent and a lipid comprising a C₁₀-C₈₀ linear, saturated or partially unsaturated, aliphatic chain. In some embodiments, the present disclosure pertains to compositions or methods related to a composition comprising a biologically active agent and a lipid comprising a C₁₀-C₈₀ linear, saturated or partially unsaturated, aliphatic chain, optionally substituted with one or more C₁₋₄ aliphatic group. In some embodiments, the present disclosure pertains to compositions or methods related to a composition comprising a biologically active agent and a lipid comprising a C₁₀-C₆₀ linear, saturated or partially unsaturated, aliphatic chain. In some embodiments, the present disclosure pertains to compositions or methods related to a composition comprising a biologically active agent and a lipid comprising a C₁₀-C₆₀ linear, saturated or partially unsaturated, aliphatic chain, optionally substituted with one or more C₁₋₄ aliphatic group. In some embodiments, the present disclosure pertains to compositions or methods related to a composition comprising a biologically active agent and a lipid comprising a C₁₀-C₄₀ linear, saturated or partially unsaturated, aliphatic chain. In some embodiments, the present disclosure pertains to compositions or methods related to a composition comprising a biologically active agent and a lipid comprising a C₁₀-C₄₀ linear, saturated or partially unsaturated, aliphatic chain, optionally substituted with one or more C₁₋₄ aliphatic group. In some embodiments, the present disclosure provides chirally controlled oligonucleotide compositions and a lipid selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the composition is suitable for delivery of the oligonucleotide to a muscle cell or tissue, or a muscle cell or tissue in a mammal (e.g., a human subject). In some embodiments, a biologically active agent is an oligonucleotide comprising one or more chiral internucleotidic linkages, and a provided composition is a chirally controlled oligonucleotide composition of the oligonucleotide. In some embodiments, a biologically active agent is an oligonucleotide comprising one or more chiral internucleotidic linkages, and a provided composition is a non-chirally controlled oligonucleotide composition of the oligonucleotide.

In some embodiments, the present disclosure pertains to a method of delivering a biologically active agent to a cell or tissue, wherein the method comprises steps of: providing a composition comprising a biologically active agent and a lipid; and contacting the cell or tissue with the composition; in some embodiments, the present disclosure pertains to a method of administering a biologically active agent to a subject, wherein the method comprises steps of: providing a composition comprising a biologically active agent and a lipid; and administering the composition to the subject. In some embodiments, a lipid comprises a C₁₀-C₈₀ linear, saturated or partially unsaturated, aliphatic chain. In some embodiments, a lipid comprises a C₁₀-C₈₀ linear, saturated or partially unsaturated, aliphatic chain, optionally substituted with one or more C₁₋₄ aliphatic group. In some embodiments, a lipid comprises a C₁₀-C₆₀ linear, saturated or partially unsaturated, aliphatic chain. In some embodiments, a lipid comprises a C₁₀-C₆₀ linear, saturated or partially unsaturated, aliphatic chain, optionally substituted with one or more C₁₋₄ aliphatic group. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In various embodiments, the lipid is selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, a biologically active agent is selected from the group consisting of: a small molecule, a peptide, a protein, a component of a CRISPR-Cas system, a carbohydrate, a therapeutic agent, a chemotherapeutic agent, a vaccine, a nucleic acid, and a lipid. In some embodiments, a nucleic acid is an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, a vector, or a portion thereof. In some embodiments, a provided composition is a chirally controlled oligonucleotide composition of a nucleic acid which comprises one or more chiral internucleotidic linkages. In various embodiments, the extra-hepatic cell or tissue is a muscle cell or tissue. In various embodiments, a muscle cell or tissue is in a subject. In various embodiments, a muscle cell or tissue is in a subject suffering from a muscle-related disease or disorder. In various embodiments, a muscle-related disorder is sarcopenia, a muscle movement disorder, a muscle wasting-related disorder, muscle degeneration, muscle weakness, muscular dystrophy, Duchenne muscular dystrophy, heart failure, breathing disorder, skeletal muscle degeneration caused by malnutrition and disease, a muscle-related disease related to impaired insulin-dependent signaling, amyotrophic lateral sclerosis, spinal muscle atrophy and spinal cord injury, ischemic muscle disease. In some embodiments, the present disclosure pertains to a method of administering a nucleic acid (as a non-limiting example, an oligonucleotide or a stereodefined oligonucleotide) to a muscle cell or tissue in a subject, wherein the subject is afflicted with a muscle-related disease or disorder, wherein the method comprises steps of: providing a composition comprising a lipid and the nucleic acid, and administering a therapeutically effective amount of the composition to the subject.

In some embodiments, a biologically active agent is an oligonucleotide, whose sequence is or comprises an element that is substantially complementary to a targeted element in a cellular nucleic acid. In some embodiments, a targeted element is or comprises a sequence element that is associated with a muscle disease, disorder or condition. In some embodiments, a muscle disease, disorder or condition is DMD. In some embodiments, a cellular nucleic acid is or comprises a transcript. In some embodiments, a cellular nucleic acid is or comprises a primary transcript. In some embodiments, a cellular nucleic acid is or comprises a genomic nucleic acid.

In some embodiments, the present disclosure provides a composition comprising a lipid and a biologically active agent.

In some embodiments, the present disclosure provides a composition comprising a lipid and a biologically active agent, characterized in that the composition delivers the biologically active agent into cells.

In some embodiments, a composition delivers the biologically active agent into the cytoplasm of the cells.

In some embodiments, a composition delivers the biologically active agent into the nucleus of the cells.

In some embodiments, the present disclosure provides a composition comprising a lipid and a biologically active agent, wherein the composition delivers the biologically active agent into cells to a level higher than that observed for the biologically active agent absent the lipid.

In some embodiments, the present disclosure provides a composition comprising a lipid and a biologically active agent, wherein the composition is characterized in that it delivers the biologically active agent into muscle cells.

In some embodiments, a composition delivers the biologically active agent into the cytoplasm of the muscle cells.

In some embodiments, a composition delivers the biologically active agent into the nucleus of the muscle cells.

In some embodiments, a composition is characterized in that when administered to a subject, the composition delivers the biologically active agent to a muscle cell in the subject.

In some embodiments, a composition delivers the biologically active agent into the cytoplasm of the muscle cells.

In some embodiments, a composition delivers the biologically active agent into the nucleus of the muscle cells.

In some embodiments, the present disclosure provides a composition for delivery of a biologically active agent to a muscle cell or tissue, comprising a lipid and the biologically active agent.

In some embodiments, the present disclosure provides a composition comprising a biologically active agent and a lipid selected from the list of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

In some embodiments, the present disclosure provides a composition comprising a biologically active agent and a lipid selected from:

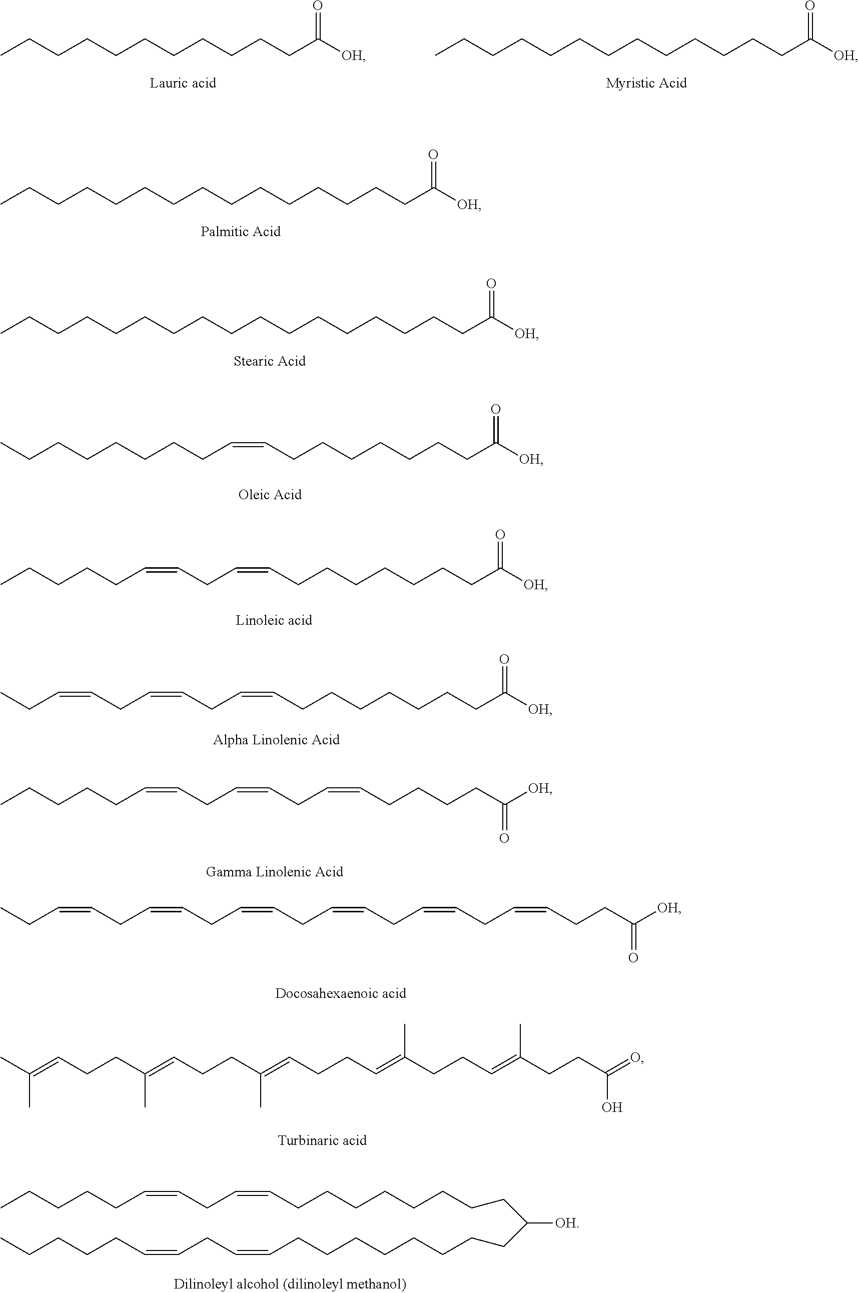

In some embodiments, the present disclosure provides a composition comprising a biologically active agent and a lipid,

wherein the lipid comprises a C₁₀-C₄₀ linear, saturated or partially unsaturated, aliphatic chain, optionally substituted with one or more C₁₋₄ aliphatic group,

wherein the biologically active agent is selected from the group consisting of: a small molecule, a peptide, a protein, a component of a CRISPR-Cas system, a carbohydrate, a therapeutic agent, a chemotherapeutic agent, a vaccine, a nucleic acid, and a lipid.

In some embodiments, the present disclosure provides a composition comprising a nucleic acid and a lipid, for delivery of the lipid to a muscle cell or tissue.

In some embodiments, the present disclosure provides an oligonucleotide composition comprising a plurality of oligonucleotides, which share:
1) a common base sequence;
2) a common pattern of backbone linkages; and
3) a common pattern of backbone phosphorus modifications;
   wherein one or more oligonucleotides of the plurality are individually conjugated to a lipid.

In some embodiments, the present disclosure provides a chirally controlled oligonucleotide composition comprising a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages;

one or more oligonucleotides of the plurality are individually conjugated to a lipid; and

one or more oligonucleotides of the plurality are optionally and individually conjugated to a targeting compound or moiety.

In some embodiments, an oligonucleotide comprises a sequence which is substantially complementary to that of a targeted element in a nucleic acid in a cell.

In some embodiments, an oligonucleotide comprises a sequence which is substantially complementary to that of a targeted element in a nucleic acid in a cell in a subject.

In some embodiments, an oligonucleotide comprises a sequence which is substantially complementary to that of a targeted element in a nucleic acid in a cell, wherein the nucleic acid is genomic.

In some embodiments, an oligonucleotide comprises a sequence which is substantially complementary to that of a targeted element in a nucleic acid in a cell in a subject, wherein the nucleic acid is genomic.

In some embodiments, an oligonucleotide comprises a sequence which is substantially complementary to that of a targeted element in a nucleic acid in a cell, wherein the targeted element is a mRNA or a portion thereof.

In some embodiments, an oligonucleotide comprises a sequence which is substantially complementary to that of a targeted element in a nucleic acid in a cell in a subject, wherein the targeted element is a mRNA or a portion thereof.

In some embodiments, an oligonucleotide comprises a sequence which is substantially complementary to that of a targeted element in a nucleic acid in a cell, wherein the targeted element is associated with a disease or disorder.

In some embodiments, an oligonucleotide comprises a sequence which is substantially complementary to that of a targeted element in a nucleic acid in a cell in a subject, wherein the targeted element is associated with a disease or disorder.

In some embodiments, an oligonucleotide comprises a sequence which is substantially complementary to that of a targeted element in a nucleic acid in a muscle cell, wherein the targeted element is associated with a muscle-related disease or disorder.

In some embodiments, an oligonucleotide comprises a sequence which is substantially complementary to that of a targeted element in a nucleic acid in a muscle cell in a subject, wherein the targeted element is associated with a muscle-related disease or disorder.

In some embodiments, a plurality of oligonucleotides share the same stereochemistry at five or more chiral internucleotidic linkages.

In some embodiments, a plurality of oligonucleotides share the same stereochemistry at ten or more chiral internucleotidic linkages.

In some embodiments, a plurality of oligonucleotides share the same stereochemistry at each of the chiral internucleotidic linkages so that they share a common pattern of backbone chiral centers.

In some embodiments, one or more oligonucleotides of the plurality are independently conjugated to a lipid through a 5′-OH on the oligonucleotide.

In some embodiments, one or more oligonucleotides of the plurality are independently conjugated to a lipid through a 3′-OH on the oligonucleotide.

In some embodiments, each oligonucleotide of the plurality is individually conjugated to a lipid.

In some embodiments, each oligonucleotide of the plurality is individually conjugated to the same lipid.

In some embodiments, the present disclosure provides a composition comprising a biologically active agent and a lipid, wherein the agent is any agent disclosed herein, and wherein the lipid is any lipid disclosed herein.

In some embodiments, the present disclosure provides a method of delivering an oligonucleotide to a muscle cell or tissue in a human subject, comprising:
(a) Providing a composition or method of any one embodiment; and
(b) Administering the composition to the human subject such that the oligonucleotide is delivered to a muscle cell or tissue in the subject.

In some embodiments, the present disclosure provides a method for delivering a biologically active agent to a muscle cell or tissue comprising preparing a composition according to any one of the embodiments and treating [contacting] the cell or tissue with the composition.

In some embodiments, the present disclosure provides a method of modulating the level of a transcript or gene product of a gene in a cell, the method comprising the step of contacting the cell with a composition according to any one of the embodiments, wherein the biologically active agent is capable of modulating the level of the transcript or gene product.

In some embodiments, the present disclosure provides a method for inhibiting expression of a gene in a muscle cell or tissue comprising preparing a composition according to any one of the embodiments and treating the muscle cell or tissue with the composition.

In some embodiments, the present disclosure provides a method for inhibiting expression of a gene in a muscle cell or tissue in a mammal comprising preparing a composition according to any one of the embodiments and administering the composition to the mammal.

In some embodiments, the present disclosure provides a method of treating a disease that is caused by the over-expression of one or several proteins in a muscle cell or tissue in a subject, said method comprising the administration of a composition according to any one of the embodiments to the subject.

In some embodiments, the present disclosure provides a method of treating a disease that is caused by a reduced, suppressed or missing expression of one or several proteins in a subject, said method comprising the administration of a composition according to any one of the embodiments to the subject.

In some embodiments, the present disclosure provides a method for generating an immune response in a subject, said method comprising the administration of a composition according to any one of the embodiments to the subject, wherein the biologically active compound is an immunomodulating nucleic acid.

In some embodiments, the present disclosure provides a method for treating a sign and/or symptom of a disease, disorder, or condition in a subject selected from cancer, a proliferative disease, disorder, or condition, a metabolic disease, disorder, or condition, an inflammatory disease, disorder, or condition, and a viral infection by providing a composition or method of any one of the embodiments and administering the composition to the subject.

In some embodiments, the present disclosure provides a method of modulating the amount of exon skipping in a cell, the method comprising the step of contacting the cell with a composition according to any one of the embodiments, wherein the biologically active agent is capable of modulating the amount of exon skipping.

In some embodiments, the present disclosure provides a method of administering a biologically active agent to a subject in need thereof, comprising steps of providing a composition comprising the agent a lipid, and administering the composition to the subject, wherein the agent is any agent disclosed herein, and wherein the lipid is any lipid disclosed herein.

In some embodiments, the present disclosure provides a method of treating a disease in a subject, the method comprising steps of providing a composition comprising the agent a lipid, and administering a therapeutically effective amount of the composition to the subject, wherein the agent is any agent disclosed herein, and wherein the lipid is any lipid disclosed herein, and wherein the disease is any disease disclosed herein.

In some embodiments, a lipid comprises an optionally substituted C₁₀-C₄₀ saturated or partially unsaturated aliphatic chain.

In some embodiments, a lipid comprises an optionally substituted C₁₀-C₄₀ linear, saturated or partially unsaturated, aliphatic chain.

In some embodiments, a lipid comprises a C₁₀-C₄₀ linear, saturated or partially unsaturated, aliphatic chain, optionally substituted with one or more C₁₋₄ aliphatic group.

In some embodiments, a lipid comprises an unsubstituted C₁₀-C₄₀ linear, saturated or partially unsaturated, aliphatic chain.

In some embodiments, a lipid comprises no more than one optionally substituted C₁₀-C₄₀ linear, saturated or partially unsaturated, aliphatic chain.

In some embodiments, a lipid comprises two or more optionally substituted C₁₀-C₄₀ linear, saturated or partially unsaturated, aliphatic chain.

In some embodiments, a lipid comprises no tricyclic or polycyclic moiety.

In some embodiments, a lipid has the structure of R¹—COOH, wherein R¹ is an optionally substituted C₁₀-C₄₀ saturated or partially unsaturated aliphatic chain.

In some embodiments, a lipid is conjugated through its carboxyl group.

In some embodiments, a lipid is selected from:

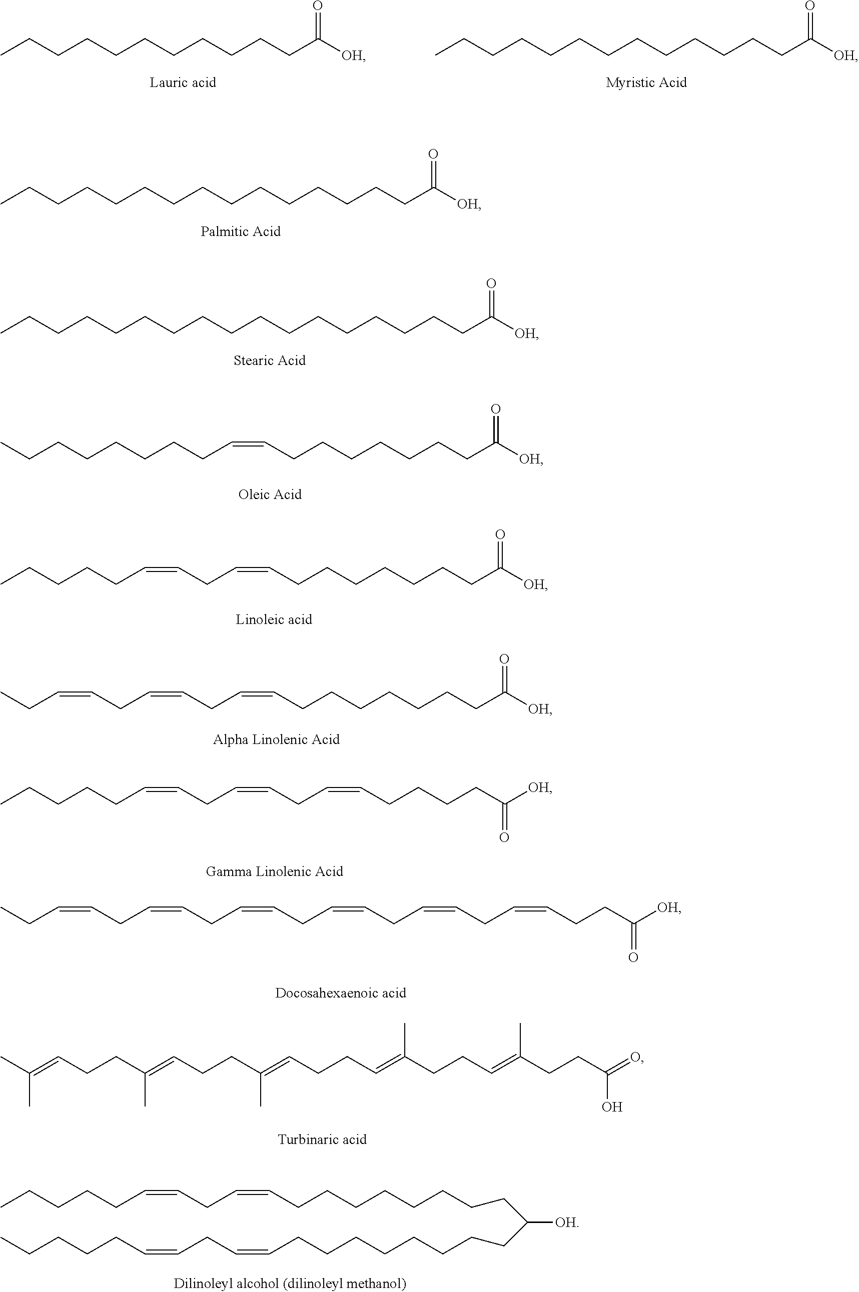

In some embodiments, a lipid is conjugated to the biologically active agent.

In some embodiments, a lipid is directly conjugated to the biologically active agent.

In some embodiments, a lipid is conjugated to the biologically active agent via a linker.

In some embodiments, a linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; and a linker comprising at least one peptide-based cleavage group.

In some embodiments, a nucleic acid is an oligonucleotide, an antisense oligonucleotide, an RNAi agent, a miRNA, splice switching oligonucleotide (SSO), immunomodulatory nucleic acid, an aptamer, a ribozyme, a mRNA, a lncRNA, a ncRNA, an antigomir (e.g., an antagonist to a miRNA, lncRNA, ncRNA or other nucleic acid), a plasmid, a vector, or a portion thereof.

In some embodiments, a RNAi agent is a siRNA, a shRNA, a miRNA, a sisiRNA, a meroduplex RNA (mdRNA), a DNA-RNA chimera, a siRNA comprising two mismatches (or more mismatches), a neutral siRNA, an aiRNA, or a siRNA comprising a terminal or internal spacer.

In some embodiments, each oligonucleotide of the plurality is individually conjugated to the same lipid at the same location.

In some embodiments, a lipid is conjugated to an oligonucleotide through a linker.

In some embodiments, one or more oligonucleotides of the plurality are independently conjugated to a targeting compound or moiety.

In some embodiments, one or more oligonucleotides of the plurality are independently conjugated to a lipid and a targeting compound or moiety.

In some embodiments, one or more oligonucleotides of the plurality are independently conjugated to a lipid at one end and a targeting compound or moiety at the other.

In some embodiments, oligonucleotides of the plurality share the same chemical modification patterns.

In some embodiments, oligonucleotides of the plurality share the same chemical modification patterns comprising one or more base modifications.

In some embodiments, oligonucleotides of the plurality share the same chemical modification patterns comprising one or more sugar modifications.

In some embodiments, a common base sequence is capable of hybridizing with a transcript in a muscle cell, which transcript contains a mutation that is linked to a muscle disease, or whose level, activity and/or distribution is linked to a muscle disease.

In some embodiments, a common base sequence is capable of hybridizing with a transcript in a muscle cell, and the composition is characterized in that when it is contacted with the transcript in a transcript splicing system, splicing of the transcript is altered relative to that observed under reference conditions selected from the group consisting of absence of the composition, presence of a reference composition, and combinations thereof.

In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).

In some embodiments, a common base sequence hybridizes with a transcript of dystrophin.

In some embodiments, a common base sequence hybridizes with a transcript of dystrophin, and the composition increases the production of one or more functional or partially functional proteins encoded by dystrophin.

In some embodiments, an oligonucleotide or oligonucleotides is or are splice switching oligonucleotide or oligonucleotides.

In some embodiments, a plurality of oligonucleotides share a common pattern of sugar modification, which comprises 3 or more 2′-F.

In some embodiments, a plurality of oligonucleotides share a common pattern of sugar modification, which comprises 3 or more consecutive 2′-F.

In some embodiments, a plurality of oligonucleotides share a common pattern of sugar modification, which comprises 3 or more consecutive 2′-F within the 10 nucleotide at the 5′-end.

In some embodiments, a plurality of oligonucleotides share a common pattern of sugar modification, which comprises 3 or more 2′-F within the 10 nucleotide at the 5′-end.

In some embodiments, a plurality of oligonucleotides share a common pattern of sugar modification, which comprises 3 or more consecutive 2′-F at the 5′-end.

In some embodiments, a plurality of oligonucleotides share a common pattern of sugar modification, which comprises 5 or more consecutive 2′-F within the first 10 nucleotide at the 3′-end.

In some embodiments, a plurality of oligonucleotides share a common pattern of sugar modification, which comprises 5 or more 2′-F within the 10 nucleotide at the 3′-end.

In some embodiments, a plurality of oligonucleotides share a common pattern of sugar modification, which comprises 7 or more consecutive 2′-F at the 3′-end.

In some embodiments, a plurality of oligonucleotides share a common pattern of sugar modification, which comprises 3 or more consecutive 2′-F at the 5′-end, 3 or more consecutive 2′-F at the 3′-end, and 3 or more 2′-OR between the 5′-end 2′-F and the 3′-end 2′-F modifications.

In some embodiments, a plurality of oligonucleotides share a common pattern of sugar modification, which comprises 3 or more 2′-F at the 5′-end, 3 or more 2′-F at the 3′-end, and 3 or more 2′-OR between the 5′-end 2′-F and the 3′-end 2′-F modifications.

In some embodiments, a plurality of oligonucleotides share a common pattern of sugar modification, which comprises 5 or more 2′-F within the 10 nucleotides at the 5′-end.

In some embodiments, a plurality of oligonucleotides share a common pattern of sugar modification, which comprises 3 or more consecutive 2′-F at the 5′-end.

In some embodiments, a plurality of oligonucleotides share a common pattern of sugar modification, which comprises 7 or more 2′-F within the 10 nucleotides at the 3′-end.

In some embodiments, a plurality of oligonucleotides share a common pattern of sugar modification, which comprises 5 or more consecutive 2′-F within the 10 nucleotides at the 3′-end.

In some embodiments, a plurality of oligonucleotides share a common pattern of sugar modification, which comprises 7 or more consecutive 2′-F at the 3′-end.

In some embodiments, a plurality of oligonucleotides comprises a 5′-wing-core-wing-3′ structure, wherein each wing region independently comprises 3 to 10 nucleosides, and the core region independently comprises 3 to 10 nucleosides.

In some embodiments, a core comprises at least one internucleotidic linkage which is chirally controlled (e.g., a phosphorothioate in Sp or Rp configuration) and at least one internucleotidic linkage which is not chiral (e.g., a phosphodiester or phosphorodithioate). In some embodiments, a core comprises at least one internucleotidic linkage which is chirally controlled phosphorothioate in Sp configuration and at least one internucleotidic linkage which is not chiral (e.g., a phosphodiester or phosphorodithioate). In some embodiments, each wing region comprises no modified sugar moieties. In some embodiments, a core region comprises one or more natural phosphate linkages. In some embodiments, each internucleotidic linkage following a core nucleoside is a natural phosphate linkage. In some embodiments, a wing comprises at least one internucleotidic linkage which is chirally controlled (e.g., a phosphorothioate in Sp or Rp configuration) and at least one internucleotidic linkage which is not chiral (e.g., a phosphodiester or phosphorodithioate). In some embodiments, a wing comprises at least one internucleotidic linkage which is chirally controlled phosphorothioate in Sp configuration and at least one internucleotidic linkage which is not chiral (e.g., a phosphodiester or phosphorodithioate). In some embodiments, a wing comprises one or more modified internucleotidic linkages. In some embodiments, each internucleotidic linkage following a core nucleoside is a modified internucleotidic linkage.

In some embodiments, a 5′-wing region comprises 3 or more 2′-F.

In some embodiments, a 5′-wing region comprises 3 or more consecutive 2′-F.

In some embodiments, a 5′-wing region comprises 10% or more 2′-F.

In some embodiments, each sugar of a 5′-wing region comprises a 2′-F.

In some embodiments, a 5′-wing region comprises 3 or more chiral internucleotidic linkages.

In some embodiments, a 5′-wing region comprises 3 or more consecutive internucleotidic linkages.

In some embodiments, a 5′-wing region comprises 10% or more internucleotidic linkages.

In some embodiments, each internucleotidic linkage of a 5′-wing region is chiral.

In some embodiments, each internucleotidic linkage of a 5′-wing region is a phosphorothioate linkage.

In some embodiments, a 5′-wing region comprises 5 or more Rp chiral internucleotidic linkages.

In some embodiments, a 5′-wing region comprises 5 or more Rp consecutive internucleotidic linkages.

In some embodiments, a 5′-wing region comprises 10% or more Rp internucleotidic linkages.

In some embodiments, each internucleotidic linkage of a 5′-wing region is Rp.

In some embodiments, a 3′-wing region comprises 3 or more 2′-F.

In some embodiments, a 3′-wing region comprises 5 or more consecutive 2′-F.

In some embodiments, a 3′-wing region comprises 10% or more 2′-F.

In some embodiments, each sugar of a 3′-wing region comprises a 2′-F.

In some embodiments, a 3′-wing region comprises 3 or more chiral internucleotidic linkages.

In some embodiments, a 3′-wing region comprises 5 or more consecutive internucleotidic linkages.

In some embodiments, a 3′-wing region comprises 10% or more internucleotidic linkages.

In some embodiments, each internucleotidic linkage of a 3′-wing region is chiral.

In some embodiments, each internucleotidic linkage of a 3′-wing region is a phosphorothioate linkage.

In some embodiments, a 3′-wing region comprises 3 or more Rp chiral internucleotidic linkages.

In some embodiments, a 3′-wing region comprises 5 or more Rp consecutive internucleotidic linkages.

In some embodiments, a 3′-wing region comprises 10% or more Rp internucleotidic linkages.

In some embodiments, each internucleotidic linkage of a 3′-wing region is Rp.

In some embodiments, a 5′-wing and the 3′-wing have the same length, pattern of chemical modifications, pattern of backbone internucleotidic linkages, and pattern of backbone chiral centers.

In some embodiments, an internucleotidic linkage between the 5′-wing region and the core region is a chiral internucleotidic linkage.

In some embodiments, an internucleotidic linkage between the 5′-wing region and the core region is a phosphorothioate linkage.

In some embodiments, an internucleotidic linkage between the 5′-wing region and the core region is an Rp phosphorothioate linkage.

In some embodiments, an internucleotidic linkage between the 3′-wing region and the core region is a chiral internucleotidic linkage.

In some embodiments, an internucleotidic linkage between the 3′-wing region and the core region is a phosphorothioate linkage.

In some embodiments, an internucleotidic linkage between the 3′-wing region and the core region is an Rp phosphorothioate linkage.

In some embodiments, a core region comprises 3 or more 2′-OR.

In some embodiments, a core region comprises 5 or more consecutive 2′-OR.

In some embodiments, a core region comprises 10% or more 2′-OR.

In some embodiments, each sugar of a core region comprises a 2′-OR.

In some embodiments, a core region comprises 3 or more chiral internucleotidic linkages.

In some embodiments, a core region comprises 5 or more consecutive internucleotidic linkages.

In some embodiments, a core region comprises 10% or more internucleotidic linkages.

In some embodiments, each internucleotidic linkage of a core region is chiral.

In some embodiments, each internucleotidic linkage of a core region is a phosphorothioate linkage.

In some embodiments, a core region comprises 3 or more Sp chiral internucleotidic linkages.

In some embodiments, a core region comprises 5 or more Sp consecutive internucleotidic linkages.

In some embodiments, a core region comprises 10% or more Sp internucleotidic linkages.

In some embodiments, each internucleotidic linkage of a core region is Sp.

In some embodiments, a 5′-wing region comprises 5 or more Sp chiral internucleotidic linkages.

In some embodiments, a 5′-wing region comprises 5 or more Sp consecutive internucleotidic linkages.

In some embodiments, a 5′-wing region comprises 10% or more Sp internucleotidic linkages.

In some embodiments, each internucleotidic linkage of a 5′-wing region is Sp.

In some embodiments, a 3′-wing region comprises 3 or more Sp chiral internucleotidic linkages.

In some embodiments, a 3′-wing region comprises 5 or more Sp consecutive internucleotidic linkages.

In some embodiments, a 3′-wing region comprises 10% or more Sp internucleotidic linkages.

In some embodiments, each internucleotidic linkage of a 3′-wing region is Sp.

In some embodiments, an internucleotidic linkage between the 5′-wing region and the core region is an Sp phosphorothioate linkage.

In some embodiments, an internucleotidic linkage between the 3′-wing region and the core region is an Sp phosphorothioate linkage.

In some embodiments, a nucleic acid is a splice switching oligonucleotide (SSO).

In some embodiments, a nucleic acid is a splice switching oligonucleotide (SSO) which targets dystrophin.

In some embodiments, a nucleic acid is a splice switching oligonucleotide (SSO) which targets dystrophin exon 51, 45, 53 or 44.

In some embodiments, a nucleic acid is a splice switching oligonucleotide (SSO) which targets dystrophin exon 51.

In some embodiments, an immunomodulatory nucleic acid is a CpG oligonucleotide.

In some embodiments, an immunomodulatory nucleic acid is a CpG oligonucleotide which is capable of agonizing an immune response which is TLR9-mediated or TLR9-associated.

In some embodiments, an immunomodulatory nucleic acid is a CpG oligonucleotide which is capable of antagonizing an immune response which is TLR9-mediated or TLR9-associated.

In some embodiments, an oligonucleotide comprises a strand of about 14 to about 49 nucleotides.

Where the oligonucleotide further comprises a second strand.

In some embodiments, an oligonucleotide comprises at least one modification to a base, sugar or internucleoside linkage.

In some embodiments, a modification is a sugar modifications at the 2′ carbon.

In some embodiments, a modification is a sugar modifications at the 2′ carbon selected from: 2′-MOE, 2′-OMe, and 2′-F.

In some embodiments, a biologically active agent is a nucleic acid.

In some embodiments, a biologically active agent is an immunomodulatory nucleic acid.

In some embodiments, a biologically active agent is a CpG oligonucleotide that agonizes or antagonizes an immune response

In some embodiments, a biologically active agent is an CpG oligonucleotide that agonizes or antagonizes an immune response which is TLR9-mediated or TLR9-associated.

In some embodiments, a biologically active agent is a small molecule, and wherein the small molecule is hydrophobic

In some embodiments, a biologically active agent is a hydrophobic small molecule selected from the group consisting of a sterol and a hydrophobic vitamin.

In some embodiments, a biologically active agent is cholesterol.

In some embodiments, a biologically active agent is a protein selected from the group consisting of a nucleoprotein, a mucoprotein, a lipoprotein, a synthetic polypeptide, a small molecule linked to a protein and a glycoprotein.

In some embodiments, a biologically active agent is a nucleic acid in the form of a single stranded or partially double stranded oligomer or a polymer composed of ribonucleotides.

In some embodiments, a biologically active agent is a nucleic acid selected from the group consisting of miRNA, antisense oligonucleotides, siRNA, immune-stimulatory oligonucleotides, aptamers, Piwi-interacting RNAs (piRNAs), small nucleolar RNAs (snoRNAs), ribozymes, and plasmids encoding a specific gene or siRNA.

In some embodiments, a cell or tissue is a muscle cell or tissue.

In some embodiments, a biologically active agent is a nucleic acid.

In some embodiments, a biologically active agent is an oligonucleotide.

In some embodiments, a biologically active agent is an oligonucleotide which mediates exon skipping.

In some embodiments, a biologically active agent is a stereodefined oligonucleotide which mediates exon skipping.

In some embodiments, a disease or disorder is a muscle-related disease or disorder.

In some embodiments, a muscle-related disorder is sarcopenia, a muscle movement disorder, a muscle wasting-related disorder, muscle degeneration, muscle weakness, muscular dystrophy, Duchenne muscular dystrophy, heart failure, breathing disorder, skeletal muscle degeneration caused by malnutrition and disease, a muscle-related disease related to impaired insulin-dependent signaling, amyotrophic lateral sclerosis, spinal muscle atrophy and spinal cord injury, ischemic muscle disease.

In some embodiments, a cell or tissue is a muscle cell or tissue, wherein the biologically active agent is a stereodefined oligonucleotide which is a splice switching oligonucleotide, and wherein the subject is afflicted with a muscle disorder.

In some embodiments, a cell or tissue is a muscle cell or tissue, wherein the biologically active agent is a stereodefined oligonucleotide which is a splice switching oligonucleotide, and wherein the subject is afflicted with muscular dystrophy.

In some embodiments, a cell or tissue is a muscle cell or tissue, wherein the biologically active agent is a stereodefined oligonucleotide which is a splice switching oligonucleotide, and wherein the subject is afflicted with Duchenne muscular dystrophy.

In some embodiments, a lipid comprises an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₈₀ saturated or partially unsaturated, aliphatic chain.

In some embodiments, a lipid comprises an optionally substituted C₁₀-C₈₀ linear, saturated or partially unsaturated, aliphatic chain.

In some embodiments, a lipid comprises an optionally substituted C₁₀-C₆₀ saturated or partially unsaturated, aliphatic chain.

In some embodiments, a lipid comprises an optionally substituted C₁₀-C₆₀ linear, saturated or partially unsaturated, aliphatic chain.

In some embodiments, a lipid comprises an optionally substituted C₁₀-C₄₀ saturated or partially unsaturated, aliphatic chain.

In some embodiments, a lipid comprises an optionally substituted C₁₀-C₄₀ linear, saturated or partially unsaturated, aliphatic chain.

In some embodiments, a lipid comprises an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₈₀ saturated or partially unsaturated, aliphatic chain.

In some embodiments, a lipid comprises an optionally substituted C₁₀-C₆₀ linear, saturated or partially unsaturated, aliphatic chain.

In some embodiments, a lipid comprises an optionally substituted C₁₀-C₄₀ linear, saturated or partially unsaturated, aliphatic chain.

In some embodiments, a lipid comprises an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₄₀ saturated or partially unsaturated, aliphatic chain.

In some embodiments, a lipid comprises an optionally substituted C₁₀-C₄₀ linear, saturated or partially unsaturated, aliphatic chain.

In some embodiments, a composition further comprises one or more additional components selected from: a polynucleotide, carbonic anhydrase inhibitor, a dye, an intercalating agent, an acridine, a cross-linker, psoralene, mitomycin C, a porphyrin, TPPC4, texaphyrin, Sapphyrin, a polycyclic aromatic hydrocarbon phenazine, dihydrophenazine, an artificial endonuclease, a chelating agent, EDTA, an alkylating agent, a phosphate, an amino, a mercapto, a PEG, PEG-40K, MPEG, [MPEG]₂, a polyamino, an alkyl, a substituted alkyl, a radiolabeled marker, an enzyme, a hapten biotin, a transport/absorption facilitator, aspirin, vitamin E, folic acid, a synthetic ribonuclease, a protein, a glycoprotein, a peptide, a molecule having a specific affinity for a co-ligand, an antibody, a hormone, a hormone receptor, a non-peptidic species, a lipid, a lectin, a carbohydrate, a vitamin, a cofactor, or a drug.

In some embodiments, a lipid comprises a C₁₀-C₈₀ linear, saturated or partially unsaturated, aliphatic chain.

In some embodiments, a composition further comprises a linker linking the biologically active agent and the lipid, wherein the linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group.

In some embodiments, a biologically active agent comprises or consists of or is an oligonucleotide or oligonucleotide composition or chirally controlled oligonucleotide composition.

In some embodiments, a biologically active agent comprises or consists of or is an oligonucleotide or oligonucleotide composition or chirally controlled oligonucleotide composition, wherein the sequence of the oligonucleotide comprises or consists of the sequence of any oligonucleotide described herein.

In some embodiments, a biologically active agent comprises or consists of or is an oligonucleotide or oligonucleotide composition or chirally controlled oligonucleotide composition, wherein the sequence of the oligonucleotide comprises or consists of the sequence of any oligonucleotide listed in Table 4A.

In some embodiments, a biologically active agent comprises or consists of or is an oligonucleotide or oligonucleotide composition or chirally controlled oligonucleotide composition, wherein the sequence of the oligonucleotide comprises or consists of the sequence of a splice-switching oligonucleotide.

In some embodiments, a biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of an exon in the dystrophin gene.

In some embodiments, a biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of exon 51 in the dystrophin gene.

In some embodiments, a biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of exon 51, 45, 53 or 44 in the dystrophin gene.

In some embodiments, a biologically active agent comprises or consists of or is an oligonucleotide or oligonucleotide composition or chirally controlled oligonucleotide composition, wherein the sequence of the oligonucleotide comprises or consists of the sequence of any of: WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2531, WV-2533, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546.

In some embodiments, a common base sequence is UCAAGGAAGAUGGCAUUUCU (SEQ ID NO: 1). In some embodiments, a common base sequence comprises UCAAGGAAGAUGGCAUUUCU (SEQ ID NO: 1), and the oligonucleotide has a length of up to 30 bases. In some embodiments, a common base sequence comprises UCAAGGAAGAUGGCAUUUCU (SEQ ID NO: 1), and the oligonucleotide has a length of up to 40 bases. In some embodiments, a common base sequence comprises UCAAGGAAGAUGGCAUUUCU (SEQ ID NO: 1), and the oligonucleotide has a length of up to 50 bases. In some embodiments, a common base sequence comprises at least 15 contiguous bases of UCAAGGAAGAUGGCAUUUCU (SEQ ID NO: 1), and the oligonucleotide has a length of up to 30 bases. In some embodiments, a common base sequence comprises at least 15 contiguous bases of UCAAGGAAGAUGGCAUUUCU (SEQ ID NO: 1), and the oligonucleotide has a length of up to 40 bases. In some embodiments, a common base sequence comprises at least 15 contiguous bases of UCAAGGAAGAUGGCAUUUCU (SEQ ID NO: 1), and the oligonucleotide has a length of up to 50 bases. In some embodiments, a common base sequence comprises a sequence having no more than 5 mismatches from the sequence of bases of UCAAGGAAGAUGGCAUUUCU (SEQ ID NO: 1), and the oligonucleotide has a length of up to 30 bases. In some embodiments, a common base sequence comprises a sequence having no more than 5 mismatches from the sequence of bases of UCAAGGAAGAUGGCAUUUCU (SEQ ID NO: 1), and the oligonucleotide has a length of up to 40 bases. In some embodiments, a common base sequence comprises a sequence having no more than 5 mismatches from the sequence of bases of UCAAGGAAGAUGGCAUUUCU (SEQ ID NO: 1), and the oligonucleotide has a length of up to 50 bases.

In some embodiments, a common base sequence is UCAAGGAAGAUGGCAUUUCU (SEQ ID NO: 1), and a common pattern of backbone chiral centers comprises at least one chirally controlled center. In some embodiments, a common base sequence comprises UCAAGGAAGAUGGCAUUUCU (SEQ ID NO: 1), the oligonucleotide has a length of up to 30 bases, and a common pattern of backbone chiral centers comprises at least one chirally controlled center. In some embodiments, a common base sequence comprises UCAAGGAAGAUGGCAUUUCU (SEQ ID NO: 1), and the oligonucleotide has a length of up to 40 bases, and a common pattern of backbone chiral centers comprises at least one chirally controlled center. In some embodiments, a common base sequence comprises UCAAGGAAGAUGGCAUUUCU (SEQ ID NO: 1), the oligonucleotide has a length of up to 50 bases, and a common pattern of backbone chiral centers comprises at least one chirally controlled center. In some embodiments, a common base sequence comprises at least 15 contiguous bases of UCAAGGAAGAUGGCAUUUCU (SEQ ID NO: 1), the oligonucleotide has a length of up to 30 bases, and a common pattern of backbone chiral centers comprises at least one chirally controlled center. In some embodiments, a common base sequence comprises at least 15 contiguous bases of UCAAGGAAGAUGGCAUUUCU (SEQ ID NO: 1), the oligonucleotide has a length of up to 40 bases, and a common pattern of backbone chiral centers comprises at least one chirally controlled center. In some embodiments, a common base sequence comprises at least 15 contiguous bases of UCAAGGAAGAUGGCAUUUCU (SEQ ID NO: 1), the oligonucleotide has a length of up to 50 bases, and a common pattern of backbone chiral centers comprises at least one chirally controlled center. In some embodiments, a common base sequence comprises a sequence having no more than 5 mismatches from the sequence of bases of UCAAGGAAGAUGGCAUUUCU (SEQ ID NO: 1), the oligonucleotide has a length of up to 30 bases, and a common pattern of backbone chiral centers comprises at least one chirally controlled center. In some embodiments, a common base sequence comprises a sequence having no more than 5 mismatches from the sequence of bases of UCAAGGAAGAUGGCAUUUCU (SEQ ID NO: 1), the oligonucleotide has a length of up to 40 bases, and a common pattern of backbone chiral centers comprises at least one chirally controlled center. In some embodiments, a common base sequence comprises a sequence having no more than 5 mismatches from the sequence of bases of UCAAGGAAGAUGGCAUUUCU (SEQ ID NO: 1), the oligonucleotide has a length of up to 50 bases, and a common pattern of backbone chiral centers comprises at least one chirally controlled center.

In some embodiments, a common base sequence is UCAAGGAAGAUGGCAUUUCU (SEQ ID NO: 1), and a common pattern of backbone chiral centers comprises at least one chirally controlled center which is a phosphorothioate in the Sp configuration. In some embodiments, a common base sequence comprises UCAAGGAAGAUGGCAUUUCU (SEQ ID NO: 1), the oligonucleotide has a length of up to 30 bases, and a common pattern of backbone chiral centers comprises at least one chirally controlled center which is a phosphorothioate in the Sp configuration. In some embodiments, a common base sequence comprises UCAAGGAAGAUGGCAUUUCU (SEQ ID NO: 1), and the oligonucleotide has a length of up to 40 bases, and a common pattern of backbone chiral centers comprises at least one chirally controlled center which is a phosphorothioate in the Sp configuration. In some embodiments, a common base sequence comprises UCAAGGAAGAUGGCAUUUCU (SEQ ID NO: 1), the oligonucleotide has a length of up to 50 bases, and a common pattern of backbone chiral centers comprises at least one chirally controlled center which is a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 30 bases, and a common pattern of backbone chiral centers comprises at least one chirally controlled center which is a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 40 bases, and a common pattern of backbone chiral centers comprises at least one chirally controlled center which is a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 50 bases, and a common pattern of backbone chiral centers comprises at least one chirally controlled center which is a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 30 bases, and a common pattern of backbone chiral centers comprises at least one chirally controlled center which is a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 40 bases, and a common pattern of backbone chiral centers comprises at least one chirally controlled center which is a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 50 bases, and a common pattern of backbone chiral centers comprises at least one chirally controlled center which is a phosphorothioate in the Sp configuration.

In some embodiments, a common base sequence is UCAAGGAAGAUGGCAUUUCU (SEQ ID NO: 1), and a common pattern of backbone chiral centers comprises at least three chirally controlled centers. In some embodiments, a common base sequence comprises UCAAGGAAGAUGGCAUUUCU (SEQ ID NO: 1), the oligonucleotide has a length of up to 30 bases, and a common pattern of backbone chiral centers comprises at least three chirally controlled centers. In some embodiments, a common base sequence comprises UCAAGGAAGAUGGCAUUUCU (SEQ ID NO: 1), and the oligonucleotide has a length of up to 40 bases, and a common pattern of backbone chiral centers comprises at least three chirally controlled centers. In some embodiments, a common base sequence comprises UCAAGGAAGAUGGCAUUUCU (SEQ ID NO: 1), the oligonucleotide has a length of up to 50 bases, and a common pattern of backbone chiral centers comprises at least three chirally controlled centers. In some embodiments, a common base sequence comprises at least 15 contiguous bases of UCAAGGAAGAUGGCAUUUCU (SEQ ID NO: 1), the oligonucleotide has a length of up to 30 bases, and a common pattern of backbone chiral centers comprises at least three chirally controlled centers. In some embodiments, a common base sequence comprises at least 15 contiguous bases of UCAAGGAAGAUGGCAUUUCU (SEQ ID NO: 1), the oligonucleotide has a length of up to 40 bases, and a common pattern of backbone chiral centers comprises at least three chirally controlled centers. In some embodiments, a common base sequence comprises at least 15 contiguous bases of UCAAGGAAGAUGGCAUUUCU (SEQ ID NO: 1), the oligonucleotide has a length of up to 50 bases, and a common pattern of backbone chiral centers comprises at least three chirally controlled centers. In some embodiments, a common base sequence comprises a sequence having no more than 5 mismatches from the sequence of bases of UCAAGGAAGAUGGCAUUUCU (SEQ ID NO: 1), the oligonucleotide has a length of up to 30 bases, and a common pattern of backbone chiral centers comprises at least three chirally controlled centers. In some embodiments, a common base sequence comprises a sequence having no more than 5 mismatches from the sequence of bases of UCAAGGAAGAUGGCAUUUCU (SEQ ID NO: 1), the oligonucleotide has a length of up to 40 bases, and a common pattern of backbone chiral centers comprises at least three chirally controlled centers. In some embodiments, a common base sequence comprises a sequence having no more than 5 mismatches from the sequence of bases of UCAAGGAAGAUGGCAUUUCU (SEQ ID NO: 1), the oligonucleotide has a length of up to 50 bases, and a common pattern of backbone chiral centers comprises at least three chirally controlled centers.

In some embodiments, a common base sequence is UCAAGGAAGAUGGCAUUUCU (SEQ ID NO: 1), and a common pattern of backbone chiral centers comprises at least five chirally controlled centers which are each a phosphorothioate in the Sp configuration. In some embodiments, a common base sequence comprises UCAAGGAAGAUGGCAUUUCU (SEQ ID NO: 1), the oligonucleotide has a length of up to 30 bases, and a common pattern of backbone chiral centers comprises at least five chirally controlled centers which are each a phosphorothioate in the Sp configuration. In some embodiments, a common base sequence comprises UCAAGGAAGAUGGCAUUUCU (SEQ ID NO: 1), and the oligonucleotide has a length of up to 40 bases, and a common pattern of backbone chiral centers comprises at least five chirally controlled centers which are each a phosphorothioate in the Sp configuration. In some embodiments, a common base sequence comprises UCAAGGAAGAUGGCAUUUCU (SEQ ID NO: 1), the oligonucleotide has a length of up to 50 bases, and a common pattern of backbone chiral centers comprises at least five chirally controlled centers which are each a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 30 bases, and a common pattern of backbone chiral centers comprises at least five chirally controlled centers which are each a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 40 bases, and a common pattern of backbone chiral centers comprises at least five chirally controlled centers which are each a phosphorothioate in the Sp configuration. In some embodiments, a common base sequence comprises at least 15 contiguous bases of UCAAGGAAGAUGGCAUUUCU (SEQ ID NO: 1), the oligonucleotide has a length of up to 50 bases, and a common pattern of backbone chiral centers comprises at least five chirally controlled centers which are each a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 30 bases, and a common pattern of backbone chiral centers comprises at least five chirally controlled centers which are each a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 40 bases, and a common pattern of backbone chiral centers comprises at least five chirally controlled centers which are each a phosphorothioate in the Sp configuration. In some embodiments, a common base sequence comprises a sequence having no more than 5 mismatches from the sequence of bases of UCAAGGAAGAUGGCAUUUCU (SEQ ID NO: 1), the oligonucleotide has a length of up to 50 bases, and a common pattern of backbone chiral centers comprises at least five chirally controlled centers which are each a phosphorothioate in the Sp configuration.

In some embodiments, a common pattern of backbone chiral centers is selected from: SSS, SSSS, SSSSS, SOS, SSOSS, SSSOSSS, SSSSOSSSS, SSSSSOSSSSS, SSSSSSOSSSSSS, SSSSSSSOSSSSSSS, SSSSSSSSOSSSSSSSS, SSSSSSSSSOSSSSSSSSS, SOSOSOSOS, SSOSOSOSOSS, SSSOSOSOSOSSS, SSSSOSOSOSOSSSS, SSSSSOSOSOSOSSSSS, SSSSSSOSOSOSOSSSSSS, SOSOSSOOS, SSOSOSSOOSS, SSSOSOSSOOSSS, SSSSOSOSSOOSSSS, SSSSSOSOSSOOSSSSS, SSSSSSOSOSSOOSSSSSS, SOSOOSOOS, SSOSOOSOOSS, SSSOSOOSOOSSS, SSSSOSOOSOOSSSS, SSSSSOSOOSOOSSSSS, SSSSSSOSOOSOOSSSSSS, SOSOSSOOS, SSOSOSSOOSO, SSSOSOSSOOSOS, SSSSOSOSSOOSOSS, SSSSSOSOSSOOSOSSS, SSSSSSOSOSSOOSOSSSS, SOSOOSOOSO, SSOSOOSOOSOS, SSSOSOOSOOSOS, SSSSOSOOSOOSOSS, SSSSSOSOOSOOSOSSS, SSSSSSOSOOSOOSOSSSS, SSOSOSSOO, SSSOSOSSOOS, SSSSOSOSSOOS, SSSSSOSOSSOOSS, SSSSSSOSOSSOOSSS, OSSSSSSOSOSSOOSSS, OOSSSSSSOSOSSOOS, OOSSSSSSOSOSSOOSS, OOSSSSSSOSOSSOOSSS, OOSSSSSSOSOSSOOSSSS, OOSSSSSSOSOSSOOSSSSS, and OOSSSSSSOSOSSOOSSSSSS, wherein O is a non-chiral center and S is a chiral center in a Sp configuration. In some embodiments, the non-chiral center is phosphodiester. In some embodiments, the chiral center in a Sp configuration is a phosphorothioate.

In some embodiments, a sequence of an oligonucleotide includes any one or more of: base sequence (including length); pattern of chemical modifications to sugar and base moieties; pattern of backbone linkages; pattern of natural phosphate linkages, phosphorothioate linkages, phosphorothioate triester linkages, and combinations thereof, pattern of backbone chiral centers; pattern of stereochemistry (Rp/Sp) of chiral internucleotidic linkages; pattern of backbone phosphorus modifications; pattern of modifications on the internucleotidic phosphorus atom, such as —S—, and -L-R¹ of formula I.

In some embodiments, a muscle cell or tissue is selected from: skeletal muscle, smooth muscle, heart muscle, thoracic diaphragm, gastrocnemius, quadriceps, triceps, and/or heart.

In some embodiments, a method delivers the biologically active agent into the cytoplasm of a cell.

In some embodiments, a method delivers the biologically active agent into the nucleus of a cell.

In some embodiments, a chiral internucleoside linkage is a phosphorothioate.

In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).

### BRIEF DESCRIPTION OF THE DRAWINGS

FIG. 1. Exon skipping mediated by a biologically active agent, oligonucleotide WV-942 (SEQ ID NO: 206), delivered via gymnotic delivery (not conjugated to a lipid), or conjugated to a lipid (listed in Table 1).

FIG. 2. Example lipid conjugates.

FIG. 3. In vivo pharmacokinetic (PK) data related to delivery of oligonucleotide WV-942 delivered via gymnotic delivery (not conjugated to a lipid), or conjugated to a lipid, to gastrocnemius, heart and quadriceps muscle tissues. Tested articles are listed in Table 1.

FIG. 4. In vivo pharmacokinetic (PK) data related to delivery of WV-942 delivered via gymnotic delivery (not conjugated to a lipid), or conjugated to a lipid, to gastrocnemius, heart and quadriceps and diaphragm muscle tissues.

FIG. 5. Standard curves for lipid conjugates in different tissues (quadriceps and thoracic diaphragm).

FIG. 6. Standard curves for lipid conjugates in different tissues (heart and gastrocnemius).

FIG. 7. Example structures of lipids and linkers for conjugation to a biologically active agent. Abbreviation: Oligo: an example oligonucleotide.

FIG. 8. Hybridization assay to detect ASO: Sandwich. Abbreviations: B: biotin; SA: streptavidin; AP: alkaline phosphatase; ASO: antisense oligonucleotide.

FIG. 9A to 9E. LC-MS and deconvoluted mass of lipid conjugates of various oligonucleotides.

FIGS. 10A and 10B. Sequences and the chemistry of various oligonucleotides: WV395 (SEQ ID NO: 2432) and WV884 to WV897 (SEQ ID NOS 1159-1172, respectively). The suffices 0.01 and 0.02 indicate batch numbers. These include stereopure (chirally pure) oligonucleotides or oligonucleotide compositions, including 2′-OMe modifications. FIG. 10B discloses SEQ ID NO: 206.

FIGS. 11A and 11B. Ability of various oligonucleotides to induce skipping of exon 51 of human dystrophin. FIG. 11B is a compilation of data, including three or more replicates. Controls: WV-942, WV-1714, and untreated; Concentration: 10 uM; Duration: 4 days in differentiation medium; treatment was gymnotic (without transfection reagent); Cells: Del 48-50 [Primary human myoblasts from a patient with (dystrophin deletion exon 48-50), DL 589.2 (dystrophin deletion exon 51-55)].

FIGS. 12A and 12B. Composition of PS (phosphorothioates) and 2′-F on the wings of various oligonucleotides, including WV-2095 to WV-2109 (SEQ ID NOS: 1144-1158, respectively). WV-2106 to WV-2109 are hemimers. FIG. 12B discloses SEQ ID NO: 434.

FIGS. 13A and 13B. Ability of various oligonucleotides to induce skipping of exon 51 of dystrophin. FIG. 13B shows additional data for WV-1714 (SEQ ID NO: 434). WV-1683 (SEQ ID NO: 426), a negative control in this experiment, targets mouse exon 23.

FIGS. 14A and 14B. Sequence and chemistry of various oligonucleotides, WV-1108 (SEQ ID NO: 1226) and WV-2381 to WV-2385 (SEQ ID NOS 1268-1272, respectively). These have PS (phosphorothioates) in the wings and PO (phosphorodiesters) in the core. FIG. 14B discloses SEQ ID NO: 474.

FIG. 15. Ability of various oligonucleotides to induce skipping of exon 51 of dystrophin. Controls: WV-942 (Drisapersen, stereorandom) and untreated; Concentration: 10 uM; Duration: 4 days in differentiation medium; Cells: Del 48-50; treatment was gymnotic (without transfection reagent).

FIGS. 16A and 16B. Sequences and chemistry of various oligonucleotides, WV-2366 to WV-2370 (SEQ ID NOS 1263-1267, respectively). These have phosphorothioates in the Sp conformation in the wings and PO (phosphorodiesters) in the core.

FIG. 17. Ability of various oligonucleotides to induce skipping of exon 51 of dystrophin. Controls: WV-942 and untreated; Concentration: 10 uM; Duration: 4 days in differentiation medium; Cells: Del 48-50; treatment was gymnotic (without transfection reagent).

FIG. 18. Sequences and chemistry of various oligonucleotides, which are 20-mers or 25-mers, including WV-2313 to WV-2320 (SEQ ID NOS 1091-1098, respectively), and WV-2223 to WV-2230 (SEQ ID NOS 1005-1012, respectively).

FIG. 19. Location of the sequences of various oligonucleotides, which are 20-mers or 25-mers, including WV-2313 to WV-2320, and WV-2223 to WV-2230, relative to the human (H) and mouse (M) dystrophin sequences (SEQ ID NOS 2434 and 2433, respectively).

FIG. 20. Ability of various oligonucleotides to induce skipping of exon 51 of dystrophin. Controls: WV-942 and untreated; Concentration: 10 uM; Duration: 4 days in differentiation medium; Cells: Del 48-50; treatment was gymnotic (without transfection reagent).

FIG. 21. FIG. 21 shows the efficacy of stereopure oligonucleotides with 2′-F wings and either PO or Rp cores, in skipping exon 51 of human dystrophin, compared to WV-942 (Drisapersen). Treatment was 10 μM, gymnotic treatment.

FIG. 22. FIG. 22 shows the efficacy of stereopure oligonucleotides in skipping exon 51 of human dystrophin, compared to WV-942. Data for two different doses, 3 μM and 10 μM, are presented. On the bottom left are stereorandomers with different patterns of 2′-F and 2′-OMe modifications (SEQ ID NOS 429-436, respectively, in order of appearance). On the bottom right are stereopure oligonucleotides.

FIG. 23. FIG. 23 shows the efficacy of various oligonucleotides; shown are fold-changes compared to WV-942. Data for two different doses, 3 μM and 10 μM, are presented.

FIG. 24. FIG. 24 shows an example of a CA (carbonic anhydrase) inhibitor and an example linker, for attachment to a biologically active agent (as a non-limiting example, an oligonucleotide).

FIG. 25. FIG. 25 shows example skipping efficiency of oligonucleotides comprising lipid moieties in skipping exon 51 of human dystrophin. Data for different doses from 0.3 μM to 30 μM, are presented. Skipping efficiency generally increases with increased concentration. WV-3545 (WV-3473 conjugated to stearic acid by PO and C6 amino linker) and WV-3546 (WV-3473 conjugated to turbinaric acid by PO and C6 amino linker), both containing lipid moieties, demonstrated higher efficiency. Treatment was gymnotic (without transfection reagent). The experiment was done in triplicate, with average data shown.

FIG. 26. FIG. 26 shows that several example provided oligonucleotides do not have hTLR9 agonist activity under the tested conditions. The experiment was done in triplicate, with average data shown.

FIG. 27. FIG. 27 shows that example provided oligonucleotides comprising lipid moieties can effectively counteract hTLR9 agonistic activity (and to antagonize hTLR9). As demonstrated, conjugates of lipids (e.g., stearic acid (WV-3545) or turbinaric acid (WV-3546)) and oligonucleotides (e.g., WV-3473 (WV-3545 and WV-3546)) have significantly increased hTLR9 antagonistic activities. The concentration of agonistic oligonucleotide ODN2006 was held constant at 0.3 μM. Each oligonucleotide was tested at decreasing concentrations of: 5, 2.5, 1.25, 0.6, 0.3, 0.15 and 0.075 μM (from left to right). Treatment was gymnotic (without transfection reagent). The experiment was done in triplicate, with average data shown.

FIG. 28. FIG. 28 shows that example provided oligonucleotides comprising lipid moieties can effectively counteract hTLR9 agonistic activity (and to antagonize hTLR9). As demonstrated, conjugates of lipids (e.g., stearic acid (WV-3545) or turbinaric acid (WV-3546)) and oligonucleotides (e.g., WV-3473 (WV-3545 and WV-3546)) have significantly increased hTLR9 antagonistic activities. neg: negative control (buffer only). ODN2006c: an agonistic control in which the CpG sequence is replaced by GpC. PMO: Eteplirsen. The concentration of agonistic oligonucleotide ODN2006 was held constant at 0.3 μM. Each oligonucleotide was tested at decreasing concentrations of: 5, 2.5, 1.25, 0.6, 0.3, 0.15 and 0.075 μM (from left to right). Treatment was gymnotic (without transfection reagent). The experiment was done in triplicate, with average data shown.

FIG. 29. FIG. 29 shows that example provided oligonucleotides comprising various lipid moieties can significantly improve skipping efficiency compared to WV-942. Data for two doses, 3 μM (right column) and 10 μM (left column), are presented. Treatment was gymnotic (without transfection reagent). ND: not determined.

FIG. 30. FIG. 30 shows example skipping efficiency of example provided oligonucleotides in skipping exon 51 of human dystrophin. Lipid conjugation (WV-3534, WV-3553, WV-3546, and WV-4106) significantly improved efficiency. Skipping efficiency generally increases with increased concentration. Data for four different doses, 1 μM, 3 μM, 10 μM and 10 μM are presented. DMD del48-50 cells were used. Treatment was gymnotic (without transfection reagent). Figure discloses SEQ ID NOS 703, 721, 728, 722, and 750, respectively, in order of appearance.

FIGS. 31A to 31D. FIGS. 31A to 31D show the distribution of oligonucleotides in various muscle tissues: gastrocnemius (FIG. 31A); triceps (FIG. 31B); heart (FIG. 31C); and diaphragm (FIG. 31D). Oligonucleotides tested were: WV-3473 (SEQ ID NO: 703), WV-3545 (SEQ ID NO: 721) and WV-3546 (SEQ ID NO: 722), with WV-942 (SEQ ID NO: 206) as a control. Example oligonucleotides comprising lipid moieties have improved distributions to one or more muscle tissues, and/or may be readily cleared after a period of time compared to the control.

### DETAILED DESCRIPTION OF CERTAIN EMBODIMENTS

### 1. Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. Unless otherwise specified, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof.

Alkenyl: As used herein, the term “alkenyl” refers to an alkyl group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an alkyl group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Antibody: The terms “antibody”, “immunoglobulin” and related terms, as used herein, refer to a protein (or fragment thereof, or biologically active fragment thereof) produced mainly by plasma cells that is used by the immune system to recognize, identify and/or neutralize specific antigens, epitopes, structures, pathogens, nucleic acids and other molecules. In some embodiments, an antibody recognizes a unique molecule of the harmful agent, called an antigen, via the variable region. In some embodiments, antibodies include, without limitation: monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. In some embodiments, an antibody is a monoclonal antibody, for example, an antibody obtained from a population of substantially homogeneous antibodies. In some embodiments, an antibody is a chimeric antibody, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include, but are not limited to, “primatized” antibodies comprising variable domain antigen-binding sequences derived from a non-human primate (e.g., Old World Monkey, Ape etc.) and human constant region sequences. In some embodiments, an antibody fragment comprises a portion of an intact antibody, preferably the antigen binding and/or the variable region of the intact antibody. Non-limiting examples of antibody fragments include Fab, Fab′, F(ab′)₂ and Fv fragments; diabodies; linear antibodies; nanobodies; single-chain antibody molecules and multispecific antibodies formed from antibody fragments. In some embodiments, an antibody can be of any of five classes, IgA, IgD, IgE, IgG and IgM, and may be encoded by a mRNA, including the heavy chains designated alpha, delta, epsilon, gamma and mu, respectively. In some embodiments, any of the subclasses of antibodies may be encoded in part or in whole and include the following subclasses: IgG1, IgG2, IgG3, IgG4, IgA1 and IgA2. In various embodiments, an antibody can be utilized to treat conditions or diseases in many therapeutic areas such as, but not limited to, blood, cardiovascular, CNS, poisoning (including antivenoms), dermatology, endocrinology, gastrointestinal, medical imaging, musculoskeletal, oncology, immunology, respiratory, sensory and anti-infective. In some embodiments, an antibody is any of antibody variants, including, but not limited to, substitutional variants, conservative amino acid substitution, insertional variants, deletional variants and/or covalent derivatives. In one embodiment, the primary construct and/or mmRNA disclosed herein may encode an immunoglobulin Fc region. In another embodiment, the primary constructs and/or mmRNA may encode a variant immunoglobulin Fc region. In some embodiments, the primary constructs and/or mmRNA may encode an antibody having a variant immunoglobulin Fc region as described in U.S. Pat. No. 8,217,147.

Antisense oligonucleotide: The terms “antisense oligonucleotide” or “ASO”, as used herein, refer to an oligonucleotide or the like having, comprising, or consisting of a sequence of bases or the like which allow the oligonucleotide or the like to hybridize to a target molecule, such as another nucleic acid, modified nucleic acid or nucleic acid analog, e.g., by base-pairing, such as Watson-Crick base-pairing or non-Watson-Crick basepairing. In some embodiments, an antisense oligonucleotide is fully complementary or nearly fully complementary to the target molecule. In some embodiments, any olignucleotide of any type described herein or known in the art can be used as an antisense oligonucleotide. In various embodiments, an antisense oligonucleotide can perform or participate in any of various biological functions, including RNA interference, RNaseH-mediated cleavage, exon skipping, the prevention of exon skipping, the enhancement or blocking of an agent (e.g., a protein, RNA, protein-RNA complex, or any other molecule) from binding to another nucleic acid, or any other biological function performed by an antisense oligonucleotide, as described herein or known in the art. In some embodiments, an antisense oligonucleotide is an oligonucleotide which participates in RNaseH-mediated cleavage; for example, an antisense oligonucleotide hybridizes in a sequence-specific manner to a portion of a target mRNA, thus targeting the mRNA for cleavage my RNaseH. In some embodiments, an antisense oligonucleotide is able to differentiate between a wild-type and a mutant allele of a target. In some embodiments, an antisense oligonucleotide significantly participates in RNaseH-mediated cleavage of a mutant allele but participates in RNaseH-mediated cleavage of a wild-type allele to a much less degree (e.g., does not significantly participate in RNaseH-mediated cleavage of the wild-type allele of the target).

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, use of the term “about” in reference to dosages means±5 mg/kg/day.

Aptamer: The term “aptamer”, as used herein, refers to a nucleic acid molecule, e.g., a molecule comprising a RNA, DNA or nucleotide analog, that is capable of binding to a specific molecule with high affinity and specificity (Ellington et al., Nature 346, 818-22 (1990); and Tuerk et al., Science 249, 505-10 (1990)). In various embodiments, a ligand that binds to an aptamer includes, without limitation, small molecules, such as drugs, metabolites, intermediates, cofactors, transition state analogs, ions, metals, nucleic acids, and toxins. In some embodiments, an aptamer may also bind natural and synthetic polymers, including proteins, peptides, nucleic acids, polysaccharides, glycoproteins, hormones, receptors and cell surfaces such as cell walls and cell membranes. In some embodiments, an aptamer is between about 10 and about 300 nucleotides in length. In some embodiments, an aptamer is between about 30 and about 100 nucleotides in length. In some embodiments, an aptamer is made that bind to a wide variety of molecules. Each of these molecules can be used as a modulator of gene expression. In some embodiments, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have reportedly been used as target ligands in vitro aptamer selections (Werstuck and Green, Science 282:296-298 (1998)). Other small organic molecules like dopamine, theophylline, sulforhodamine B, and cellobiose have also been reported as ligands in the isolation of aptamers. In some embodiments, an aptamers is been isolated for antibiotics such as kanamycin A, lividomycin, tobramycin, neomycin B, viomycin, chloramphenicol and streptomycin. For a review of aptamers that recognize small molecules, see Famulok, Science 9:324-9 (1999). In some embodiments, a ligand of the aptamer of an aptamer-regulated nucleic acid of the invention is a cell-permeable, small organic molecule. Small organic molecules which do not have a general inhibitory effect on translation can be used as ligands. The small molecule can also exhibit in vivo persistence sufficient for achieving a desired level of inhibition of translation. The molecules also can be screened to identify those that are bioavailable after, for example, oral administration. In some embodiments, the ligand is nontoxic. The ligand may optionally be a drug, including, for example, a steroid. In some embodiments, in some of the methods of controlling gene expression, a ligand can be pharmacologically inert. In some embodiments, a ligand is a polypeptide whose presence in the cell is indicative of a disease or pathological condition. In other embodiments, the ligand for an aptamer is an antibiotic, such as chloramphenicol. In an alternative embodiment, the ligand of the aptamer is an organic dye such as Hoeschst dye 33258. In still another embodiment, the ligand may be a metal ion. In a specific embodiment, the aptamer domain of an aptamer-regulated nucleic acid responds to binding to caffeine. In some embodiments, an aptamers is developed to bind particular ligands by employing known in vivo or in vitro (most typically, in vitro) selection techniques known as SELEX (Ellington et al., Nature 346, 818-22 (1990); and Tuerk et al., Science 249, 505-10 (1990)). Methods of making aptamers are also described in, for example, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291. In some embodiments, aptamers include those that target any of: VEGF, tissue factor pathway inhibitor (TFPI), Factor IXa, complement component 5 (C₅), HIV Tat protein, and HIV Rev protein.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. In some embodiments, an aryl group has a radical or point of attachment on an aromatic ring.

Biologically active agent: The term “biologically active agent”, as used herein, refers to any agent (including, but not limited to, an active compound) which has, mediates, or participates in a biological activity. In various embodiments, a biologically active agent can be organic or in-organic. Non-limiting examples of biologically active agents include: a small molecule, a peptide, a protein, a component of a CRISPR-Cas system, a carbohydrate, a therapeutic agent, a chemotherapeutic agent, a vaccine, a nucleic acid, and a lipid. In some embodiments, a biologically active agent includes an inorganic or organic molecule including a small molecule, peptide (e.g. cell penetrating peptides), carbohydrate (including monosaccharides, oligosaccharides, and polysaccharides), protein (including nucleoprotein, mucoprotein, lipoprotein, synthetic polypeptide, or a small molecule linked to a protein, glycoprotein), steroid, nucleic acid, lipid, hormone, or combination thereof, that causes a biological effect when administered in vivo to an animal, including but not limited to birds and mammals, including humans. In some embodiments, the biologically active agent is charged. In some embodiments, the biologically active agent is positively charged. In some embodiments, the biologically active agent is negatively charged. In some embodiments, a biologically active agent is selected from: 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, Abamectin, abanoquil, abecarnil, abiraterone, Ablukast, Ablukast Sodium, Acadesine, acamprosate, Acarbose, Acebutolol, Acecamide Hydrochloride, Aceclidine, aceclofenae, Acedapsone, Aceglutamide Aluminum, Acemannan, Acetaminophen, Acetazolamide, Acetohexamide, Acetohydroxamic Acid, acetomepregenol, Acetophenazine Maleate, Acetosulfone Sodium, Acetylcholine Chloride, Acetylcysteine, acetyl-L-carnitine, acetylmethadol, Acifran, acipimox, acitemate, Acitretin, Acivicin, Aclarubicin, aclatonium, Acodazole Hydrochloride, aconiazide, Acrisorcin, Acrivastine, Acronine, Actisomide, Actodigin, Acyclovir, acylfulvene, adafenoxate, adapalene, Adapalene, adatanserin, Adatanserin Hydrochloride, adecypenol, adecypenol, Adefovir, adelmidrol, ademetionine, Adenosine, Adinazolam, Adipheinine Hydrochloride, adiposin, Adozelesin, adrafinil, Adrenalone, airbutamine, alacepril, Alamecin, Alanine, Alaproclate, alaptide, Albendazole, albolabrin, Albuterol, Albutoin, Alclofenae, Alclometasone Dipropionate, Alcloxa, aldecalmycin, Aldesleukin, Aldioxa, Alendronate Sodium, alendronic acid, alentemol, Alentemol Hydrobromide, Aletamine Hydrochloride, Aleuronium Chloride, Alexidine, alfacalcidol, Alfentanil Hydrochloride, alfuzosin, Algestone Acetonide, alglucerase, Aliflurane, alinastine, Alipamide, Allantoin, Allobarbital, Allopurinol, ALL-TK antagonists, Alonimid, alosetron, Alosetron Hydrochloride, Alovudine, Alpertine, Alpha Amylase, alpha idosone, Alpidem, Alprazolam, Alprenolol Hydrochloride, Alprenoxime Hydrochloride, Alprostadil, Alrestatin Sodium, Altanserin Tartrate, Alteplase, Althiazide, Altretamine, altromycin B, Alverinc Citrate, Alvircept Sudotox, Amadinone Acetate, Amantadine Hydrochloride, ambamustine, Ambomycin, Ambruticin, Ambuphylline, Ambuside, Amcinafal, Amcinonide, Amdinocillin, Amdinocillin Pivoxil, Amedalin Hydrochloride, amelometasone, Ameltolide, Amesergide, Ametantrone Acetate, amezinium metilsulfate, amfebutamone, Amfenac Sodium, Amflutizole, Amicycline, Amidephrine Mesylate, amidox, Amifloxacin, amifostine, Amikacin, Amiloride Hydrochloride, Aminacrine Hydrochloride, Aminobenzoate Potassium, Aminobenzoate Sodium, Aminocaproic Acid, Aminoglutethimide, Aminohippurate Sodium, aminolevulinic acid, Aminophylline, A minorex, Aminosalicylate sodium, Aminosalicylic acid, Amiodarone, Amiprilose Hydrochloride, Amiquinsin Hydrochloride, amisulpride, Amitraz, Amitriptyline Hydrochloride, Amlexanox, amlodipine, Amobarbital Sodium, Amodiaquine, Amodiaquine Hydrochloride, Amorolfine, Amoxapine, Amoxicillin, Amphecloral, Amphetamine Sulfate, Amphomycin, Amphotericin B, Ampicillin, ampiroxicam, Ampyzine Sulfate, Amquinate, Amrinone, aminone, amrubicin, Amsacrine, amylin, amythiamicin, Anagestone Acetate, anagrelide, Anakinra, ananain, anaritide, Anaritide Acetate, Anastrozole, Anazolene Sodium, Ancrod, andrographolide, Androstenedione, angiogenesis inhibitors, Angiotensin Amide, Anidoxime, Anileridine, Anilopam Hydrochloride, Aniracetam, Anirolac, Anisotropine Methylbromide, Anistreplase, Anitrazafen, anordrin, antagonist D, antagonist G, antarelix, Antazoline Phosphate, Anthelmycin, Anthralin, Anthramycin, antiandrogen, Acedapsone, Felbamate, antiestrogen, antineoplaston, Antipyrine, antisense oligonucleotides, apadoline, apafant, Apalcillin Sodium, apaxifylline, Apazone, aphidicolin glycinate, Apixifylline, Apomorphine Hydrochloride, apraclonidine, Apraclonidine Hydrochloride, Apramycin, Aprindine, Aprindine Hydrochloride, aprosulate sodium, Aprotinin, Aptazapine Maleate, aptiganel, apurinic acid, apurinic acid, aranidipine, Aranotin, Arbaprostil, arbekicin, arbidol, Arbutamine Hydrochloride, Arclofenin, Ardeparin Sodium, argatroban, Arginine, Argipressin Tannate, Arildone, aripiprazol, arotinolol, Arpinocid, Arteflene, Artilide Fumarate, asimadoline, aspalatone, Asparaginase, Aspartic Acid, Aspartocin, asperfuran, Aspirin, aspoxicillin, Asprelin, Astemizole, Astromicin Sulfate, asulacrine, atamestane, Atenolol, atevirdine, Atipamezole, Atiprosin Maleate, Atolide, Atorvastatin Calcium, Atosiban, Atovaquone, atpenin B, Atracurium Besylate, atrimustine, atrinositol, Atropine, Auranofin, aureobasidin A, Aurothioglucose, Avilamycin, Avoparcin, Avridine, Axid, axinastatin 1, axinastatin 2, axinastatin 3, Azabon, Azacitidinie, Azaclorzine Hydrochloride, Azaconazole, azadirachtine, Azalanstat Dihydrochloride, Azaloxan Fumarate, Azanator Maleate, Azanidazole, Azaperone, Azaribine, Azaserine, azasetron, Azatadine Maleate, Azathioprine, Azathioprine Sodium, azatoxin, azatyrosine, azelaic acid, azelastine, azelnidipine, Azepindole, Azetepa, azimilide, Azithromycin, Azlocillin, Azolimine, Azosemide, Azotomycin, Aztreonam, Azumolene Sodium, Bacampicillin Hydrochloride, baccatin III, Bacitracin, Baclofen, bacoside A, bacoside B, bactobolamine, balanol, balazipone, balhimycin, balofloxacin, balsalazide, Bambermycins, bambuterol, Bamethan Sulfate, Bamifylline Hydrochloride, Bamidazole, baohuoside 1, Barmastine, barnidipine, Basifungin, Batanopride Hydrochloride, batebulast, Batelapine Maleate, Batimastat, beauvericin, Becanthone Hydrochloride, becaplermin, becliconazole, Beclomethasone Dipropionate, befloxatone, Beinserazide, Belfosdil, Belladonna, Beloxamide, Bemesetron, Bemitradine, Bemoradan, Benapryzine Hydrochloride, Benazepril Hydrochloride, Benazeprilat, Bendacalol Mesylate, Bendazac, Bendroflumethiazide, benflumetol, benidipine, Benorterone, Benoxaprofen, Benoxaprofen, Benoxinate Hydrochloride, Benperidol, Bentazepam, Bentiromide, Benurestat, Benzbromarone, Benzethonium Chloride, Benzetimide Hydrochloride, Benzilonium Bromide, Benzindopyrine Hydrochloride, benzisoxazole, Benzocaine, benzochlorins, Benzoctamine Hydrochloride, Benzodepa, benzoidazoxan, Benzonatate, Benzoyl Peroxide, Benzoylpas Calcium, benzoylstaurosporine, Benzquinamide, Benzthiazide, benztropine, Benztropine Mesylate, Benzydamine Hydrochloride, Benzylpenicilloyl Polylysine, bepridil, Bepridil Hydrochloride, Beractant, Beraprost, Berefrine, berlafenone, bertosamil, Berythromycin, besipirdine, beta-alethine, betaclamycin B, Betamethasone, betamipron, betaxolol, Betaxolol Hydrochloride, Bethanechol Chloride, Bethanidine Sulfate, betulinic acid, bevantolol, Bevantolol Hydrochloride, Bezafibrate, bFGF inhibitor, Bialamicol Hydrochloride, Biapenem, Bicalutamide, Bicifadine Hydrochloride, Biclodil Hydrochloride, Bidisomide, bifemelane, Bifonazole, bimakalim, bimithil, Bindarit, Biniramycin, binospirone, bioxalomycin alpha2, Bipenamol Hydrochloride, Biperiden, Biphenamine Hydrochloride, biriperone, bisantrene, bisaramil, bisaziridinylspermine, bis-benzimidazole A, bis-benzimidazole B, bisnafide, Bisobrin Lactate, Bisoprolol, Bispyrithione Magsulfex, bistramide D, bistramide K, bistratene A, Bithionolate Sodium, Bitolterol Mesylate, Bivalirudin, Bizelesin, Bleomycin Sulfate, Bolandiol Dipropionate, Bolasterone, Boldenone Undecylenate, boldine, Bolenol, Bolmantalate, bopindolol, Bosentan, Boxidine, brefeldin, breflate, Brequinar Sodium, Bretazenil, Bretylium Tosylate, Brifentanil Hydrochloride, brimonidine, Brinolase, Brocresine, Brocrinat, Brofoxine, Bromadoline Maleate, Bromazepam, Bromchlorenone, Bromelains, bromfenac, Brominidione, Bromocriptine, Bromodiphenhydramine Hydrochloride, Bromoxamide, Bromperidol, Bromperidol Decanoate, Brompheniramine Maleate, Broperamole, Bropirimine, Brotizolam, Bucamide Maleate, bucindolol, Buclizine Hydrochloride, Bucromarone, Budesonide, budipine, budotitane, Buformin, Bumetamide, Bunaprolast, bunazosin, Bunolol Hydrochloride, Bupicomide, Bupivacaine Hydrochloride, Buprenorphine Hydrochloride, Bupropion Hydrochloride, Buramate, Buserelin Acetate, Buspirone Hydrochloride, Busulfan, Butabarbital, Butacetin, Butaclamol Hydrochloride, Butalbital, Butamben, Butamirate Citrate, Butaperazine, Butaprost, Butedronate Tetrasodium, butenafine, Buterizine, buthionine sulfoximine, Butikacin, Butilfenin, Butirosin Sulfate, Butixirate, butixocort propionate, Butoconazole Nitrate, Butonate, Butopamine, Butoprozine Hydrochloride, Butorphanol, Butoxamine Hydrochloride, Butriptyline Hydrochloride, Cactinomycin, Cadexomer Iodine, Caffeine, calanolide A, Calcifediol, Calcipotriene, calcipotriol, Calcitonin, Calcitriol, Calcium Undecylenate, calphostin C, Calusterone, Cambendazole, camonagrel, camptothecin derivatives, canarypox IL-2, candesartan, Candicidin, candoxatril, candoxatrilat, Caniglibose, Canrenoate Potassium, Canrenone, capecitabine, Capobenate Sodium, Capobenic Acid, Capreomycin Sulfate, capromab, capsaicin, Captopril, Capuride, Caracemide, Carbachol, Carbadox, Carbamazepine, Carbamide Peroxide, Carbantel Lauryl Sulfate, Carbaspirin Calcium, Carbazeran, carbazomycin C, Carbenicillin Potassium, Carbenoxolone Sodium, Carbetimer, carbetocin, Carbidopa, Carbidopa-Levodopa, Carbinoxamine Maleate, Carbiphene Hydrochloride, Carbocloral, Carbocysteine, Carbol-Fuchsin, Carboplatin, Carboprost, carbovir, carboxamide-amino-triazole, carboxyamidotriazole, carboxymethylated beta-1,3-glucan, Carbuterol Hydrochloride, CaRest M3, Carfentanil Citrate, Carisoprodol, Carmantadine, Carmustine, CARN 700, Camidazole, Caroxazone, carperitide, Carphenazine Maleate, Carprofen, Carsatrin Succinate, Cartazolate, carteolol, Carteolol Hydrochloride, cartilage derived inhibitor, Carubicin Hydrochloride, Carumonam Sodium, carvedilol, carvotroline, Carvotroline Hydrochloride, carzelesin, casein kinase inhibitors (ICOS), castanospermine, caurumonam, cebaracetam, cecropin B, Cedefingol, Cefaclor, Cefadroxil, Cefamandole, Cefaparole, Cefatrizine, Cefazaflur Sodium, Cefazolin, Cefbuperazone, cefcapene pivoxil, cefdaloxime pentexil tosilate, Cefdinir, cefditoren pivoxil, Cefepime, cefetamet, Cefetecol, cefixime, cefluprenam, Cefinenoxime Hydrochloride, Cefinetazole, cefminlox, cefodizime, Cefonicid Sodium, Cefoperazone Sodium, Ceforamide, cefoselis, Cefotaxime Sodium, Cefotetan, cefotiam, Cefoxitin, cefozopran, cefpimizole, Cefpiramide, cefpirome, cefpodoxime proxetil, cefprozil, Cefroxadine, cefsulodin, Ceftazidime, cefteram, ceftibuten, Ceftizoxime Sodium, ceftriaxone, Cefuroxime, celastrol, celikalim, celiprolol, cepacidiine A, Cephacetrile Sodium, Cephalexin, Cephaloglycin, Cephaloridine, Cephalothin Sodium, Cephapirin Sodium, Cephradine, cericlamine, cerivastatin, Ceronapril, certoparin sodium, Ceruletide, Cetaben Sodium, Cetalkonium Chloride, Cetamolol Hydrochloride, cetiedil, cetirizine, Cetophenicol, Cetraxate Hydrochloride, cetrorelix, Cetylpyridinium Chloride, Chenodiol, Chlophedianol Hydrochloride, Chloral Betaine, Chlorambucil, Chloramphenicol, Chlordantoin, Chlordiazepoxide, Chlorhexidine Gluconate, chlorins, Chlormadinone Acetate, chloroorienticin A, Chloroprocaine Hydrochloride, Chloropropamide, Chloroquine, chloroquinoxaline sulfonamide, Chlorothiazide, Chlorotrianisene, Chloroxine, Chloroxylenol, Chlorphenesin Carbamate, chlorpheniramine Maleate, Chlorpromazine, Chlorpropamide, Chlorprothixene, Chlortetracycline Bisulfate, Chlorthalidone, Chlorzoxazone, Cholestyramine Resin, Chromonar Hydrochloride, cibenzoline, cicaprost, Ciclafrine Hydrochloride, Ciclazindol, ciclesonide, cicletanine, Ciclopirox, Cicloprofen, cicloprolol, Cidofovir, Cidoxepin Hydrochloride, Cifenline, Ciglitazone, Ciladopa Hydrochloride, cilansetron, Cilastatin Sodium, Cilazapril, cilnidipine, Cilobamine Mesylate, cilobradine, Cilofungin, cilostazol, Cimaterol, Cimetidine, cimetropium bromide, Cinalukast, Cinanserin Hydrochloride, Cinepazet Maleate, Cinflumide, Cingestol, cinitapride, Cinnamedrine, Cinnarizine, cinolazepam, Cinoxacin, Cinperene, Cinromide, Cintazone, Cintriamide, Cioteronel, Cipamfylline, Ciprefadol Succinate, Ciprocinonide, Ciprofibrate, Ciprofloxacin, ciprostene, Ciramadol, Cirolemycin, cisapride, cisatracurium besilate, Cisconazole, Cisplatin, cis-porphyrin, cistinexine, citalopram, Citenamide, citicoline, citreamicin alpha, cladribine, Clamoxyquin Hydrochloride, Clarithromycin, clausenamide, Clavulanate Potassium, Clazolam, Clazolimine, clebopride, Clemastine, Clentiazem Maleate, Clidinium Bromide, clinafloxacin, Clindamycin, Clioquinol, Clioxamide, Cliprofen, clobazam, Clobetasol Propionate, Clobetasone Butyrate, Clocortolone Acetate, Clodanolene, Clodazon Hydrochloride, clodronic acid, Clofazimine, Clofibrate, Clofilium Phosphate, Clogestone Acetate, Clomacran Phosphate, Clomegestone Acetate, Clometherone, clomethiazole, clomifene analogues, Clominorex, Clomiphene, Clomipramine Hydrochloride, Clonazepam, Clonidine, Clonitrate, Clonixeril, Clonixin, Clopamide, Clopenthixol, Cloperidone Hydrochloride, clopidogrel, Clopimozide, Clopipazan Mesylate, Clopirac, Cloprednol, Cloprostenol Sodium, Clorazepate Dipotassium, Clorethate, Clorexolone, Cloroperone Hydrochloride, Clorprenaline Hydrochloride, Clorsulon, Clortermine Hydrochloride, Closantel, Closiramine Aceturate, Clothiapine, Clothixamide Maleate Cloticasone Propionate, Clotrimazole, Cloxacillin Benzathine, Cloxyquin, Clozapine, Cocaine, Coccidioidin, Codeine, Codoxime, Colchicine, colestimide, Colestipol Hydrochloride, Colestolone, Colforsin, Colfosceril Palmitate, Colistimethate Sodium, Colistin Sulfate, collismycin A, collismycin B, Colterol Mesylate, combretastatin A4, combretastatin analogue, complestatin, conagenin, Conorphone Hydrochloride, contignasterol, contortrostatin, Cormethasone Acetate, Corticorelin Ovine Triflutate, Corticotropin, Cortisone Acetate, Cortivazol, Cortodoxone, cosalane, costatolide, Cosyntropin, cotinine, Coumadin, Coumermycin, crambescidin 816, Crilvastatin, crisnatol, Cromitrile Sodium, Cromolyn Sodium, Crotamiton, cryptophycin 8, cucumariosid, Cuprimyxin, curacin A, curdlan sulfate, curiosin, Cyclacillin, Cyclazocine, cyclazosin, cyclic HPMPC, Cyclindole, Cycliramine Maleate, Cyclizine, Cyclobendazole, cyclobenzaprine, cyclobut A, cyclobut G, cyclocapron, Cycloguanil Pamoate, Cycloheximide, cyclopentanthraquinones, Cyclopenthiazide, Cyclopentolate Hydrochloride, Cyclophenazine Hydrochloride, Cyclophosphamide, cycloplatam, Cyclopropane, Cycloserine, cyclosin, Cyclosporine, cyclothialidine, Cyclothiazide, cyclothiazomycin, Cyheptamide, cypemycin, Cypenamine Hydrochloride, Cyprazepam, Cyproheptadine Hydrochloride, Cyprolidol Hydrochloride, cyproterone, Cyproximide, Cysteamine, Cysteine Hydrochloride, Cystine, Cytarabine, Cytarabine Hydrochloride, cytarabine Ocfosfate, cytochalasin B, cytolytic factor, cytostatin, Dacarbazine, dacliximab, dactimicin, Dactinomycin, daidzein, Daledalin Tosylate, dalfopristin, Dalteparin Sodium, Daltroban, Dalvastatin, danaparoid, Danazol, Dantrolene, daphlnodorin A, dapiprazole, dapitant, Dapoxetine Hydrochloride, Dapsone, Daptomycin, Darglitazone Sodium, darifenacin, darlucin A, Darodipine, darsidomine, Daunorubicin Hydrochloride, Dazadrol Maleate, Dazepinil Hydrochloride, Dazmegrel, Dazopride Fumarate, Dazoxiben Hydrochloride, Debrisoquin Sulfate, Decitabine, deferiprone, deflazacort, Dehydrocholic Acid, dehydrodidemnin B, Dehydroepiandrosterone, delapril, Delapril Hydrochloride, Delavirdine Mesylate, delequamine, delfaprazine, Delmadinone Acetate, delmopinol, delphinidin, Demecarium Bromide, Demeclocycline, Demecycline, Demoxepam, Denofungin, deoxypyridinoline, Depakote, deprodone, Deprostil, depsidomycin, deramciclane, dermatan sulfate, Desciclovir, Descinolone Acetonide, Desflurane, Desipramine Hydrochloride, desirudin, Deslanoside, deslorelin, desmopressin, desogestrel, Desonide, Desoximetasone, desoxoamiodarone, Desoxycorticosterone Acetate, detajmium bitartrate, Deterenol Hydrochloride, Detirelix Acetate, Devazepide, Dexamethasone, Dexamisole, Dexbrompheniramine Maleate, Dexchlorpheniramine Maleate, Dexclamol Hydrochloride, Dexetimide, Dexfenfluramine Hydrochloride, dexifosfamide, Deximafen, Dexivacaine, dexketoprofen, dexloxiglumide, Dexmedetomidine, Dexormaplatin, Dexoxadrol Hydrochloride, Dexpanthenol, Dexpemedolac, Dexpropranolol Hydrochloride, Dexrazoxane, dexsotalol, dextrin 2-sulphate, Dextroamphetamine, Dextromethorphan, Dextrorphan Hydrochloride, Dextrothyroxine Sodium, dexverapamil, Dezaguanine, dezinamide, dezocine, Diacetolol Hydrochloride, Diamocaine Cyclamate, Diapamide, Diatrizoate Meglumine, Diatrizoic Acid, Diaveridine, Diazepam, Diaziquone, Diazoxide, Dibenzepin Hydrochloride, Dibenzothiophene, Dibucaine, Dichliorvos, Dichloralphenazone, Dichlorphenamide, Dicirenone, Diclofenac Sodium, Dicloxacillin, dicranin, Dicumarol, Dicyclomine Hydrochloride, Didanosine, didemnin B, didox, Dienestrol, dienogest, Diethylcarbamazine Citrate, diethylhomospermine, diethylnorspermine, Diethylpropion Hydrochloride, Diethylstilbestrol, Difenoximide Hydrochloride, Difenoxin, Diflorasone Diacetate, Difloxacin Hydrochloride, Difluanine Hydrochloride, Diflucortolone, Diflumidone Sodium, Diflunisal, Difluprednate, Diftalone, Digitalis, Digitoxin, Digoxin, Dihexyverine Hydrochloride, dihydrexidine, dihydro-5-azacytidine, Dihydrocodeine Bitartrate, Dihydroergotamine Mesylate, Dihydroestosterone, Dihydrostreptomycin Sulfate, Dihydrotachysterol, dihydrotaxol, 9-, Dilantin, Dilevalol Hydrochloride, Diltiazem Hydrochloride, Dimefadane, Dimefline Hydrochloride, Dimenhydrinate, Dimercaprol, Dimethadione, Dimethindene Maleate, Dimethisterone, dimethyl prostaglandin A1, Dimethyl Sulfoxide, dimethylhomospermine, dimiracetam, Dimoxamine Hydrochloride, Dinoprost, Dinoprostone, Dioxadrol Hydrochloride, dioxamycin, Diphenhydramine Citrate, Diphenidol, Diphenoxylate Hydrochloride, diphenyl spiromustine, Dipivefin Hydrochloride, Dipivefrin, dipliencyprone, diprafenone, dipropylnorspermine, Dipyridamole, Dipyrithione, Dipyrone, dirithromycin, discodermolide, Disobutamide, Disofenin, Disopyramide, Disoxaril, disulfuram, Ditekiren, Divalproex Sodium, Dizocilpine Maleate, Dobutamine, docarpamine, Docebenone, Docetaxel, Doconazole, docosanol, dofetilide, dolasetron, Ebastine, ebiratide, ebrotidine, ebselen, ecabapide, ecabet, ecadotril, ecdisteron, echicetin, echistatin, Echothiophate Iodide, Eclanamine Maleate, Eclazolast, ecomustine, Econazole, ecteinascidin 722, edaravone, Edatrexate, edelfosine, Edifolone Acetate, edobacomab, Edoxudine, edrecolomab, Edrophonium Chloride, edroxyprogesteone Acetate, efegatran, eflornithine, efonidipine, egualcen, Elantrine, eleatonin, elemene, eletriptan, elgodipine, eliprodil, Elsamitrucin, eltenae, Elucaine, emalkalim, emedastine, Emetine Hydrochloride, emiglitate, Emilium Tosylate, emitefur, emoctakin, Enadoline Hydrochloride, enalapril, Enalaprilat, Enalkiren, enazadrem, Encyprate, Endralazine Mesylate, Endrysone, Enflurane, englitazone, Enilconazole, Enisoprost, Enlimomab, Enloplatin, Enofelast, Enolicam Sodium, Enoxacin, enoxacin, enoxaparin sodium, Enoxaparin Sodium, Enoximone, Enpiroline Phosphate, Enprofylline, Enpromate, entacapone, enterostatin, Enviradene, Enviroxime, Ephedrine, Epicillin, Epimestrol, Epinephrine, Epinephryl Borate, Epipropidine, Epirizole, epirubicin, Epitetracycline Hydrochloride, Epithiazide, Epoetin Alfa, Epoetin Beta, Epoprostenol, Epoprostenol Sodium, epoxymexrenone, epristeride, Eprosartan, eptastigmine, equilenin, Equilin, Erbulozole, erdosteine, Ergoloid Mesylates, Ergonovine Maleate, Ergotamine Tartrate, ersentilide, Ersofermin, erythritol, Erythrityl Tetranitrate, Erythromycin, Esmolol Hydrochloride, Esorubicin Hydrochloride, Esproquin Hydrochloride, Estazolam, Estradiol, Estramustine, estramustine analogue, Estrazinol Hydrobromide, Estriol, Estrofurate, estrogen agonists, estrogen antagonists, Estrogens, Conjugated, Estrogens, Esterified, Estrone, Estropipate, esuprone, Etafedrine Hydrochloride, Etanidazole, etanterol, Etarotene, Etazolate Hydrochloride, Eterobarb, ethacizin, Ethacrynate Sodium, Ethacrynic Acid, Ethambutol Hydrochloride, Ethamivan, Ethanolamine Oleate, Ethehlorvynol, Ether, Ethinyl estradiol, Ethiodized Oil, Ethionamide, Ethonam Nitrate, Ethopropazine Hydrochloride, Ethosuximide, Ethotoin, Ethoxazene Hydrochloride, Ethybenztropine, Ethyl Chloride, Ethyl Dibunate, Ethylestrenol, Ethyndiol, Ethynerone, Ethynodiol Diacetate, Etibendazole, Etidocaine, Etidronate Disodium, Etidronic Acid, Etifenin, Etintidine Hydrochloride, etizolam, Etodolac, Etofenamate, Etoformin Hydrochloride, Etomidate, Etonogestrel, Etoperidone Hydrochloride, Etoposide, Etoprine, Etoxadrol Hydrochloride, Etozolin, etrabamine, Etretinate, Etryptamine Acetate, Eucatropine Hydrochloride, Eugenol, Euprocin Hydrochloride, eveminomicin, Exametazime, examorelin, Exaprolol Hydrochloride, exemestane, fadrozole, faeriefungin, Famciclovir, Famotidine, Fampridine, fantofarone, Fantridone Hydrochloride, faropenem, fasidotril, fasudil, fazarabine, fedotozine, felbamate, Felbinac, Felodipine, Felypressin, Fenalamide, Fenamole, Fenbendazole, Fenbufen, Fencibutirol, Fenclofenac, Fenclonine, Fenclorac, Fendosal, Fenestrel, Fenethylline Hydrochloride, Fenfluramine Hydrochloride, Fengabine, Fenimide, Fenisorex, Fenmetozole Hydrochloride, Fenmetramide, Fenobam, Fenoctimine Sulfate, fenofibrate, fenoldopam, Fenoprofen, Fenoterol, Fenpipalone, Fenprinast Hydrochloride, Fenprostalene, Fenquizone, fenretinide, fenspiride, Fentanyl Citrate, Fentiazac, Fenticlor, fenticonazole, Fenyripol Hydrochloride, fepradinol, ferpifosate sodium, ferristene, ferrixan, Ferrous Sulfate, Dried, Ferumoxides, ferumoxsil, Fetoxylate Hydrochloride, fexofenadine, Fezolamine Fumarate, Fiacitabine, Fialuridine, Fibrinogen I 125, filgrastim, Filipin, finasteride, Flavodilol Maleate, flavopiridol, Flavoxate Hydrochloride, Flazalone, flecamide, flerobuterol, Fleroxacin, flesinoxan, Flestolol Sulfate, Fletazepam, flezelastine, flobufen, Floctafenine, flomoxef, Flordipine, florfenicol, florifenine, flosatidil, Flosequinan, Floxacillin, Floxuridine, fluasterone, Fluazacort, Flubanilate Hydrochloride, Flubendazole, Flucindole, Flucloronide, Fluconazole, Flucytosine, Fludalanine, Fludarabine Phosphate, Fludazonium Chloride, Fludeoxyglucose F 18, Fludorex, Fludrocortisone Acetate, Flufenamic Acid, Flufenisal, Flumazenil, flumecinol, Flumequine, Flumeridone, Flumethasone, Flumetramide, Flumezapine, Fluminorex, Flumizole, Flumoxonide, flunarizine, Flunidazole, Flunisolide, Flunitrazepam, Flunixin, fluocalcitriol, Fluocinolone Acetonide, Fluocinonide, Fluocortin Butyl, Fluocortolone, Fluorescein, fluorodaunorunicin hydrochloride, Fluorodopa F 18, Fluorometholone, Fluorouracil, Fluotracen Hydrochloride, Fluoxetine, Fluoxymesterone, fluparoxan, Fluperamide, Fluperolone Acetate, Fluphenazine Decanoate, flupirtine, Fluprednisolone, Fluproquazone, Fluprostenol Sodium, Fluquazone, Fluradoline Hydrochloride, Flurandrenolide, Flurazepam Hydrochloride, Flurbiprofen, Fluretofen, flurithromycin, Fluorocitabine, Fluorofamide, Fluorogestone Acetate, Fluorothyl, Fluoroxene, Fluspiperone, Fluspirilene, Fluticasone Propionate, flutrimazole, Flutroline, fluvastatin, Fluvastatin Sodium, fluvoxamine, Fluzinamide, Folic Acid, Follicle regulatory protein, Folliculostatin, Fomepizole, Fonazine Mesylate, forasartan, forfenimex, forfenirmex, formestane, Formocortal, formoterol, Fosarilate, Fosazepam, Foscarnet Sodium, fosfomycin, Fosfonet Sodium, fosinopril, Fosinoprilat, fosphenyloin, Fosquidone, Fostedil, fostriecin, fotemustine, Fuchsin, Basic, Fumoxicillin, Fungimycin, Furaprofen, Furazolidone, Furazolium Chloride, Furegrelate Sodium, Furobufen, Furodazole, Furosemide, Fusidate Sodium, Fusidic Acid, gabapentin, Gadobenate Dimeglumine, gadobenic acid, gadobutrol, Gadodiamide, gadolinium texaphyrin, Gadopentetate Dimegiumine, gadoteric acid, Gadoteridol, Gadoversetamide, galantamine, galdansetron, Galdansetron Hydrochloride, Gallamine Triethiodide, gallium nitrate, gallopamil, galocitabine, Gamfexine, gamolenic acid, Ganciclovir, ganirelix, gelatinase inhibitors, Gemcadiol, Gemcitabine, Gemeprost, Gemfibrozil, Gentamicin Sulfate, Gentian Violet, gepirone, Gestaclone, Gestodene, Gestonorone Caproate, Gestrinone, Gevotroline Hydrochloride, girisopam, glaspimod, glaucocalyxin A, Glemanserin, Gliamilide, Glibornuride, Glicetanile Sodium, Gliflumide, Glimepiride, Glipizide, Gloximonam, Glucagon, glutapyrone, glutathione inhibitors, Glutethimide, Glyburide, glycopine, glycopril, Glycopyrrolate, Glyhexamide, Glymidine Sodium, Glyoctamide, Glyparamide, Gold Au 198, Gonadoctrinins, Gonadorelin, Gonadotropins, Goserelin, Gramicidin, Granisetron, grepafloxacin, Griseofulvin, Guaiapate, Guaithylline, Guanabenz, Guanabenz Acetate, Guanadrel Sulfate, Guancydine, Guanethidine Monosulfate, Guanfacine Hydrochloride, Guanisoquin Sulfate, Guanoclor Sulfate, Guanoctine Hydrochloride, Guanoxabenz, Guanoxan Sulfate, Guanoxyfen Sulfate, Gusperimus Trihydrochloride, Halazepam, Halcinonide, halichondrin B, Halobetasol Propionate, halofantrine, Halofantrine Hydrochloride, Halofenate, Halofuginone Hydrobromide, halomon, Halopemide, Haloperidol, halopredone, Haloprogesterone, Haloprogin, Halothane, Halquinols, Hamycin, Han memopausal gonadotropins, hatomamicin, hatomarubigin A, hatomarubigin B, hatomarubigin C, hatomarubigin D, Heparin Sodium, hepsulfam, heregulin, Hetacillin, Heteronium Bromide, Hexachlorophene:Hydrogen Peroxide, Hexafluorenium Bromide, hexamethylene bisacetamide, Hexedine, Hexobendine, Hexoprenaline Sulfate, Hexylresorcinol, Histamine Phosphate, Histidine, Histoplasmin, Histrelin, Homatropine Hydrobromide, Hoquizil Hydrochloride, Human chorionic gonadotropin, Hycanthone, Hydralazine Hydrochloride, Hydralazine Polistirex, Hydrochlorothiazide, Hydrocodone Bitartrate, Hydrocortisone, Hydroflumethiazide, Hydromorphone Hydrochloride, Hydroxyamphetamine Hydrobromide, Hydroxychloroquine Sulfate, Hydroxyphenamate, Hydroxyprogesterone Caproate, Hydroxyurca, Hydroxyzine Hydrochloride, Hymecromone, Hyoscyamine, hypericin, Ibafloxacin, ibandronic acid, ibogaine, Ibopamine, ibudilast, Ibufenac, Ibuprofen, Ibutilide Fumarate, Icatibant Acetate, Ichthammol, Icotidine, idarubicin, idoxifene, Idoxuridine, idramantone, Iemefloxacin, Iesopitron, Ifetroban, Ifosfamide, Ilepeimide, illimaquinone, ilmofosine, ilomastat, Ilonidap, iloperidone, iloprost, Imafen Hydrochloride, Imazodan Hydrochloride, imidapril, imidazenil, imidazoacridones, Imidecyl Iodine, Imidocarb Hydrochloride, Imidoline Hydrochloride, Imidurea, Imiloxan Hydrochloride, Imipenem, Imipramine Hydrochloride, imiquimod, immunostimulant peptides, Impromidine Hydrochloride, Indacrinone, Indapamide, Indecamide Hydrochloride, Indeloxazine Hydrochloride, Indigotindisulfonate Sodium, indinavir, Indocyanine Green, Indolapril Hydrochloride, Indolidan, indometacin, Indomethacin Sodium, Indoprofen, indoramin, Indorenate Hydrochloride, Indoxole, Indriline Hydrochloride, inocoterone, inogatran, inolimomab, Inositol Niacinate, Insulin, interferons, interleukins, Intrazole, Intriptyline Hydrochloride, iobenguane, Iobenzamic Acid, iobitridol, Iocarmate Meglumine, Iocarmic Acid, Iocetamic Acid, Iodamide, Iodine, Iodipamide Meglumine, Iodixanol, iodoamiloride, Iodoantipyrine I 131, Iodocholesterol I 131, iododoxorubicin, Iodohippurate Sodium I 131, Iodopyracet I 125, Iodoquinol, Iodoxamate Meglumine, Iodoxamie Acid, Ioglicic Acid, Iofetamine Hydrochloride I 123, iofratol, Ioglucol, Ioglucomide, Ioglycamic Acid, Iogulamide, Iohexyl, iomeprol, Iomethin I 125, Iopamidol, Iopanoic Acid, iopentol, Iophendylate, Ioprocemic Acid, iopromide, Iopronic Acid, Iopydol, Iopydone, iopyrol, Iosefamic Acid, Ioseric Acid, Iosulamide Meglumine, Iosumetic Acid, Iotasul, Iotetric Acid, Iothalamate Sodium, Iothalamic Acid, iotriside, Iotrolan, Iotroxic Acid, Iotyrosine I 131, Ioversol, Ioxagiate Sodium, Ioxaglate Meglumine, Ioxaglic Acid, ioxilan, Ioxotrizoic Acid, ipazilide, ipenoxazone, ipidacrine, Ipodate Calcium, ipomeanol, 4-, Ipratropium Bromide, ipriflavone, Iprindole, Iprofenin, Ipronidazole, Iproplatin, Iproxamine Hydrochloride, ipsapirone, irbesartan, irinotecan, irloxacin, iroplact, irsogladine, Irtemazole, isalsteine, Isamoxole, isbogrel, Isepamicin, isobengazole, Isobutamben, Isocarboxazid, Isoconazole, Isoetharine, isofloxythepin, Isoflupredone Acetate, Isoflurane, Isofluorophate, isohomohalicondrin B, Isoleucine, Isomazole Hydrochloride, Isomylamine Hydrochloride, Isoniazid, Isopropamide Iodide, Isopropyl Alcohol, isopropyl unoprostone, Isoproterenol Hydrochloride, Isosorbide, Isosorbide Mononitrate, Isotiquimide, Isotretinoin, Isoxepac, Isoxicam, Isoxsuprine Hydrochloride, isradipine, itameline, itasetron, Itazigrel, itopride, Itraconazole, Ivermectin, jasplakinolide, Josamycin, kahalalide F, Kalafungin, Kanamycin Sulfate, Ketamine Hydrochloride, Ketanserin, Ketazocine, Ketazolam, Kethoxal, Ketipramine Fumarate, Ketoconazole, Ketoprofen, Ketorfanol, ketorolac, Ketotifen Fumarate, Kitasamycin, Labetalol Hydrochloride, Lacidipine, lacidipine, lactitol, lactivicin, laennec, lafutidine, lamellarin-N triacetate, lamifiban, Lamivudine, Lamotrigine, lanoconazole, Lanoxin, lanperisone, lanreotide, Lansoprazole, latanoprost, lateritin, laurocapram, Lauryl Isoquinolinium Bromide, Lavoltidine Succinate, lazabemide, Lecimibide, leinamycin, lemildipine, leminoprazole, lenercept, Leniquinsin, lenograstim, Lenperone, lentinan sulfate, leptin, leptolstatin, lercanidipine, Lergotrile, lerisetron, Letimide Hydrochloride, letrazuril, letrozole, Leucine, leucomyzin, Leuprolide Acetate, leuprolide+estrogen+progesterone-, leuprorelin, Levamfetamine Succinate, levamisole, Levdobutamine Lactobionate, Leveromakalim, levetiracetam, Leveycloserine, levobetaxolol, levobunolol, levobupivacaine, levocabastine, levocarnitine, Levodopa, levodropropizine, levofloxacin, Levofuraltadone, Levoleucovorin Calcium, Levomethadyl Acetate, Levomethadyl Acetate Hydrochloride, levomoprolol, Levonantradol Hydrochloride, Levonordefrin, Levonorgestrel, Levopropoxyphene Napsylate, Levopropylcillin Potassium, levormeloxifene, Levorphanol Tartrate, levosimendan, levosulpiride, Levothyroxine Sodium, Levoxadrol Hydrochloride, Lexipafant, Lexithromycin, liarozole, Libenzapril, Lidamidine Hydrochloride, Lidocaine, Lidofenin, Lidoflazine, Lifarizine, Lifibrate, Lifibrol, Linarotene, Lincomycin, linear polyamine analogue, Linogliride, Linopirdine, linotroban, linsidomine, lintitript, lintopride, Liothyronine I 125, liothyronine sodium, Liotrix, lirexapride, lisinopril, lissoclinamide 7, Lixazinone Sulfate, lobaplatin, Lobenzarit Sodium, Lobucavir, Lodelaben, lodoxamide, Lofemizole Hydrochloride, Lofentanil Oxalate, Lofepramine Hydrochloride, Lofexidine Hydrochloride, lombricine, Lomefloxacin, lomerizine, Lometraline Hydrochloride, lometrexol, Lomofungin, Lomoxicam, Lomustine, Lonapalene, lonazolac, lonidamine, Loperamide Hydrochloride, loracarbef, Lorajmine Hydrochloride, loratadine, Lorazepam, Lorbamate, Lorcamide Hydrochloride, Loreclezole, Loreinadol, lorglumide, Lormetazepam, Lornoxicam, lornoxicam, Lortalamine, Lorzafone, losartan, losigamone, losoxantrone, Losulazine Hydrochloride, loteprednol, lovastatin, loviride, Loxapine, Loxoribine, lubeluzole, Lucanthone Hydrochloride, Lufironil, Lurosetron Mesylate, lurtotecan, luteinizing hormone, lutetium, Lutrelin Acetate, luzindole, Lyapolate Sodium, Lycetamine, lydicamycin, Lydimycin, Lynestrenol, Lypressin, Lysine, lysofylline, lysostaphin, lytic peptides, Maduramicin, Mafenide, magainin 2 amide, Magnesium Salicylate, Magnesium Sulfate, magnolol, maitansine, Malethamer, mallotochromene, mallotojaponin, Malotilate, malotilate, mangafodipir, manidipine, maniwamycin A, Mannitol, mannostatin A, manumycin E, manumycin F, mapinastine, Maprotiline, marimastat, Martek 158708, Martek 92211, Masoprocol, maspin, massetolide, matrilysin inhibitors, Maytansine, Mazapertine Succiniate, Mazindol, Mebendazole, Mebeverine Hydrochloride, Mebrofenin, Mebutamate, Mecamylamine Hydrochloride, Mechlorethamine Hydrochloride, Meclocycline, Meclofenamate Sodium, Mecloqualone, Meclorisone Dibutyrate, Medazepam Hydrochloride, Medorinone, Medrogestone, Medroxalol, Medroxyprogesterone, Medrysone, Meelizine Hydrochloride, Mefenamic Acid, Mefenidil, Mefenorex Hydrochloride, Mefexamide, Mefloquine Hydrochloride, Mefruside, Megalomicin Potassium Phosphate, Megestrol Acetate, Meglumine, Meglutol, Melengestrol Acetate, Melitracen Hydrochloride, Melphalan, Memotine Hydrochloride, Menabitan Hydrochloride, Menoctone, menogaril, Menotropins, Meobentine Sulfate, Mepartricin, Mepenzolate Bromide, Meperidine Hydrochloride, Mephentermine Sulfate, Mephenyloin, Mephobarbital, Mepivacaine Hydrochloride, Meprobamate, Meptazinol Hydrochloride, Mequidox, Meralein Sodium, merbarone, Mercaptopurine, Mercufenol Chloride, Mercury, Ammoniated, Merisoprol Hg 197, Meropenem, Mesalamine, Meseclazone, Mesoridazine, Mesterolone, Mestranol, Mesuprine Hydrochloride, Metalol Hydrochloride, Metaproterenol Polistirex, Metaraminol Bitartrate, Metaxalone, Meteneprost, meterelin, Metformin, Methacholine Chloride, Methacycline, Methadone Hydrochloride, Methadyl Acetate, Methalthiazide, Methamphetamine Hydrochloride, Methaqualone, Methazolamide, Methdilazine, Methenamine, Methenolone Acetate, Methetoin, Methicillin Sodium, Methimazole, methioninase, Methionine, Methisazone, Methixene Hydrochloride, Methocarbamol, Methohexital Sodium, Methopholine, Methotrexate, Methotrimeprazine, methoxatone, Methoxyflurane, Methsuximide, Methyclothiazide, Methyl 10 Palmoxirate, Methylatropine Nitrate, Methylbenzethonium Chloride, Methyldopa, Methyldopate Hydrochloride, Methylene Blue, Methylergonovine Maleate, methylhistamine, R-alpha, methylinosine monophosphate, Methylphenidate Hydrochloride, Methylprednisolone, Methyltestosterone, Methynodiol Diacelate, Methysergide, Methysergide Maleate, Metiamide, Metiapine, Metioprim, metipamide, Metipranolol, Metizoline Hydrochloride, Metkephamid Acetate, metoclopramide, Metocurine Iodide, Metogest, Metolazone, Metopimazine, Metoprine, Metoprolol, Metoquizine, Metrifonate, Metrizamide, Metrizoate Sodium, Metronidazole, Meturedepa, Metyrapone, Metyrosine, Mexiletine Hydrochloride, Mexrenoate Potassium, Mezlocillin, mfonelic Acid, Mianserin Hydrochloride, mibefradil, Mibefradil Dihydrochloride, Mibolerone, michellamine B, Miconazole, microcolin A, Midaflur, Midazolam Hydrochloride, midodrine, mifepristone, Mifobate, miglitol, milacemide, milameline, mildronate, Milenperone, Milipertine, milnacipran, Milrinone, miltefosine, Mimbane Hydrochloride, minaprine, Minaxolone, Minocromil, Minocycline, Minoxidil, Mioflazine Hydrochloride, miokamycin, mipragoside, mirfentanil, mirimostim, Mirincamycin Hydrochloride, Mirisetron Maleate, Mirtazapine, mismatched double stranded RNA, Misonidazole, Misoprostol, Mitindomide, Mitocarcin, Mitocromin, Mitogillin, mitoguazone, mitolactol, Mitomalcin, Mitomycin, mitonafide, Mitosper, Mitotane, mitoxantrone, mivacurium chloride, mivazerol, mixanpril, Mixidine, mizolastine, mizoribine, Moclobemide, modafinil, Modaline Sulfate, Modecamide, moexipril, mofarotene, Mofegiline Hydrochloride, mofezolac, molgramostim, Molinazone, Molindone Hydrochloride, Molsidomine, mometasone, Monatepil Maleate, Monensin, Monoctanoin, Montelukast Sodium, montirelin, mopidamol, moracizine, Morantel Tartrate, Moricizine, Morniflumate, Morphine Sulfate, Morrhuate Sodium, mosapramine, mosapride, motilide, Motretinide, Moxalactam Disodium, Moxazocine, moxiraprine, Moxnidazole, moxonidine, Mumps Skin Test Antigen, mustard anticancer agent, Muzolimine, mycaperoxide B, Mycophenolic Acid, myriaporone, Nabazenil, Nabilone, Nabitan Hydrochloride, Naboctate Hydrochloride, Nabumetone, N-acetyldinaline, Nadide, nadifloxacin, Nadolol, nadroparin calcium, nafadotride, nafamostat, nafarelin, Nafcillin Sodium, Nafenopin, Nafimidone Hydrochloride, Naflocort, Nafomine Malate, Nafoxidine Hydrochloride, Nafronyl Oxalate, Naftifine Hydrochloride, naftopidil, naglivan, nagrestip, Nalbuphine Hydrochloride, Nalidixate Sodium, Nalidixic Acid, nalmefene, Nalmexone Hydrochloride, naloxone+pentazocine, Naltrexone, Namoxyrate, Nandrolone Phenpropionate, Nantradol Hydrochloride, Napactadine Hydrochloride, napadisilate, Napamezole Hydrochloride, napaviin, Naphazoline Hydrochloride, naphterpin, Naproxen, Naproxol, napsagatran, Naranol Hydrochloride, Narasin, naratriptan, nartograstim, nasaruplase, Natamycin, nateplase, Naxagolide Hydrochloride, Nebivolol, Nebramycin, nedaplatin, Nedocromil, Nefazodone Hydrochloride, Neflumozide Hydrochloride, Nefopam Hydrochloride, Nelezaprine Maleate, Nemazoline Hydrochloride, nemorubicin, Neomycin Palmitate, Neostigmine Bromide, neridronic acid, Netilmicin Sulfate, neutral endopeptidase, Neutramycin, Nevirapine, Nexeridine Hydrochloride, Niacin, Nibroxane, Nicardipine Hydrochloride, Nicergoline, Niclosamide, Nicorandil, Nicotinyl Alcohol, Nifedipine, Nifirmerone, Nifluridide, Nifuradene, Nifuraldezone, Nifuratel, Nifuratrone, Nifurdazil, Nifurimide, Nifurpirinol, Nifurquinazol, Nifurthiazole, nilutamide, Nilvadipine, Nimazone, Nimodipine, niperotidine, niravoline, Niridazole, nisamycin, Nisbuterol Mesylate, nisin, Nisobamate, Nisoldipine, Nisoxetine, Nisterime Acetate, Nitarsone, nitazoxamide, nitecapone, Nitrafudam Hydrochloride, Nitralamine Hydrochloride, Nitramisole Hydrochloride, Nitrazepam, Nitrendipine, Nitrocycline, Nitrodan, Nitrofurantoin, Nitrofurazone, Nitroglycerin, Nitromersol, Nitromide, Nitromifene Citrate, Nitrous Oxide, nitroxide antioxidant, nitrullyn, Nivazol, Nivimedone Sodium, Nizatidine, Noberastine, Nocodazole, Nogalamycin, Nolinium Bromide, Nomifensine Maleate, Noracymethadol Hydrochloride, Norbolethone, Norepinephrine Bitartrate, Norethindrone, Norethynodrel, Norfloxacin, Norflurane, Norgestimate, Norgestomet, Norgestrel, Nortriptyline Hydrochloride, Noscapine, Novobiocin Sodium, N-substituted benzaimides, Nufenoxole, Nylestriol, Nystatin, O6-benzylguanine, Obidoxime Chloride, Ocaperidone, Ocfentanil Hydrochloride, Ocinaplon, Octanoic Acid, Octazamide, Octenidine Hydrochloride, Octodrine, Octreotide, Octriptyline Phosphate, Ofloxacin, Oformine, okicenone, Olanzapine, oligonucleotides, olopatadine, olprinone, olsalazine, Olsalazine Sodium, Olvanil, omeprazole, onapristone, ondansetron, Ontazolast, Oocyte maturation inhibitor, Opipramol Hydrochloride, oracin, Orconazole Nitrate, Orgotein, Orlislat, Ormaplatin, Ormetoprim, Ornidazole, Orpanoxin, Orphenadrine Citrate, osaterone, otenzepad, Oxacillin Sodium, Oxagrelate, oxaliplatin, Oxamarin Hydrochloride, oxamisole, Oxamniquine, oxandrolone, Oxantel Pamoate, Oxaprotiline Hydrochloride, Oxaprozin, Oxarbazole, Oxatomide, oxaunomycin, Oxazepam, oxcarbazepine, Oxendolone, Oxethazaine, Oxetorone Fumarate, Oxfendazole, Oxfenicine, Oxibendazole, oxiconazole, Oxidopamine, Oxidronic Acid, Oxifungin Hydrochloride, Oxilorphan, Oximonam, Oximonam Sodium, Oxiperomide, oxiracetam, Oxiramide, Oxisuran, Oxmetidine Hydrochloride, oxodipine, Oxogestone Phenpropionate, Oxolinic Acid, Oxprenolol Hydrochloride, Oxtriphylline, Oxybutynin Chloride, Oxychlorosene, Oxycodone, Oxymetazoline Hydrochloride, Oxymetholone, Oxymorphone Hydrochloride, Oxypertine, Oxyphenbutazone, Oxypurinol, Oxytetracycline, Oxytocin, ozagrel, Ozolinone, Paclitaxel, palauamine, Paldimycin, palinavir, palmitoylrhizoxin, Palmoxirate Sodium, pamaqueside, Pamatolol Sulfate, pamicogrel, Pamidronate Disodium, pamidronic acid, Panadiplon, panamesine, panaxytriol, Pancopride, Pancuronium Bromide, panipenem, pannorin, panomifene, pantethine, pantoprazole, Papaverine Hydrochloride, parabactin, Parachlorophenol, Paraldehyde, Paramethasone Acetate, Paranyline Hydrochloride, Parapenzolate Bromide, Pararosaniline Pamoate, Parbendazole, Parconazole Hydrochloride, Paregoric, Pareptide Sulfate, Pargyline Hydrochloride, parnaparin sodium, Paromomycin Sulfate, Paroxetine, parthenolide, Partricin, Paulomycin, pazelliptine, Pazinaclone, Pazoxide, pazufloxacin, pefloxacin, pegaspargase, Pegorgotein, Pelanserin Hydrochloride, peldesine, Peliomycin, Pelretin, Pelrinone Hydrochloride, Pemedolac, Pemerid Nitrate, pemirolast, Pemoline, Penamecillin, Penbutolol Sulfate, Penciclovir, Penfluridol, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentabamate, Pentaerythritol Tetranitrate, pentafuside, pentamidine, pentamorphone, Pentamustine, Pentapiperium Methylsulfate, Pentazocine, Pentetic Acid, Pentiapine Maleate, pentigetide, Pentisomicin, Pentizidone Sodium, Pentobarbital, Pentomone, Pentopril, pentosan, pentostatin, Pentoxifylline, Pentrinitrol, pentrozole, Peplomycin Sulfate, Pepstatin, perflubron, perfofamide, Perfosfamide, pergolide, Perhexyline Maleate, perillyl alcohol, Perindopril, perindoprilat, Perlapine, Permethrin, perospirone, Perphenazine, Phenacemide, phenaridine, phenazinomycin, Phenazopyridine Hydrochloride, Phenbutazone Sodium Glycerate, Phencarbamide, Phencyclidine Hydrochloride, Phendimetrazine Tartrate, Phenelzine Sulfate, Phenmetrazine Hydrochloride, Phenobarbital, Phenoxybenzamine Hydrochloride, Phenprocoumon, phenserine, phensuccinal, Phensuximide, Phentermine, Phentermine Hydrochloride, phentolamine mesilate, Phentoxifylline, Phenyl Aminosalicylate, phenylacetate, Phenylalanine, phenylalanyl ketoconazole, Phenylbutazone, Phenylephrine Hydrochloride, Phenylpropanolamine Hydrochloride, Phenylpropanolamine Polistirex, Phenyramidol Hydrochloride, Phenyloin, phosphatase inhibitors, Physostigmine, picenadol, picibanil, Picotrin Diolamine, picroliv, picumeterol, pidotimod, Pifamine, Pilocarpine, pilsicamide, pimagedine, Pimetine Hydrochloride, pimilprost, Pimobendan, Pimozide, Pinacidil, Pinadoline, Pindolol, pinnenol, pinocebrin, Pinoxepin Hydrochloride, pioglitazone, Pipamperone, Pipazethate, pipecuronium bromide, Piperacetazine, Piperacillin Sodium, Piperamide Maleate, piperazine, Pipobroman, Piposulfan, Pipotiazine Palmitate, Pipoxolan Hydrochloride, Piprozolin, Piquindone Hydrochloride, Piquizil Hydrochloride, Piracetam, Pirandamine Hydrochloride, pirarubicin, Pirazmonam Sodium, Pirazolac, Pirbenicillin Sodium, Pirbuterol Acetate, Pirenperone, Pirenzepine Hydrochloride, piretanide, Pirfenidone, Piridicillin Sodium, Piridronate Sodium, Piriprost, piritrexim, Pirlimycin Hydrochloride, pirlindole, pirmagrel, Pirmenol Hydrochloride, Pirnabine, Piroctone, Pirodavir, pirodomast, Pirogliride Tartrate, Pirolate, Pirolazamide, Piroxantrone Hydrochloride, Piroxicam, Piroximone, Pirprofen, Pirquinozol, Pirsidomine, Prenylamine, Pituitary, Posterior, Pivampicillin Hydrochloride, Pivopril, Pizotyline, placetin A, platinum compounds, platinum-triamine complex, Plicamycin, Plomestane, Pobilukast Edamine, Podofilox, Poisonoak Extract, Poldine Methylsulfate, Poliglusam, Polignate Sodium, Polymyxin B Sulfate, Polythiazide, Ponalrestat, Porfimer Sodium, Porfiromycin, Potassium Chloride, Potassium Iodide, Potassium Permanganate, Povidone-Iodine, Practolol, Pralidoxime Chloride, Pramiracetam Hydrochloride, Pramoxine Hydrochloride, Pranolium Chloride, Pravadoline Maleate, Pravastatin (Pravachol), Prazepam, Prazosin, Prazosin Hydrochloride, Prednazate, Prednicarbate, Prednimustine, Prednisolone, Prednisone, Prednival, Pregnenolone Succiniate, Prenalterol Hydrochloride, Pridefine Hydrochloride, Prifelone, Prilocalne Hydrochloride, Prilosec, Primaquine Phosphate, Primidolol, Primidone, Prinivil, prinomide Tromethamine, Prinoxodan, Prizidilol Hydrochloride, Proadifen Hydrochloride, Probenecid, Probicromil Calcium, Probucol, Procainamide Hydrochloride, Procaine Hydrochloride, Procarbazine Hydrochloride, Procaterol Hydrochloride, Prochlorperazine, Procinonide, Proclonol, Procyclidine Hydrochloride, Prodilidine Hydrochloride, Prodolic Acid, Profadol Hydrochloride, Progabide, Progesterone, Proglumide, Proinsulin Human, Proline, Prolintane Hydrochloride, Promazine Hydrochloride, Promethazine Hydrochloride, Propafenone Hydrochloride, propagermanium, Propanidid, Propantheline Bromide, Proparacaine Hydrochloride, Propatyl Nitrate, propentofylline, Propenzolate Hydrochloride, Propikacin, Propiomazine, Propionic Acid, propionylcarnitine, L-, propiram, propiram+paracetamol, propiverine, Propofol, Propoxycaine Hydrochloride, Propoxyphene Hydrochloride, Propranolol Hydrochloride, Propulsid, propyl bis-acridone, Propylhexedrine, Propyliodone, Propylthiouracil, Proquazone, Prorenoate Potassium, Proroxan Hydrochloride, Proscillaridin, Prostalene, prostratin, Protamine Sulfate, protegrin, Protirelin, protosufloxacin, Protriptyline Hydrochloride, Proxazole, Proxazole Citrate, Proxicromil, Proxorphan Tartrate, prulifloxacin, Pseudoephedrine Hydrochloride, Puromycin, purpurins, Pyrabrom, Pyrantel, Pamoate, Pyrazinamide, Pyrazofurin, pyrazoloacridine, Pyridostigmine Bromide, Pyrilamine Maleate, Pyrimethamine, Pyrinoline, Pyrithione Sodium, Pyrithione Zinc, Pyrovalerone Hydrochloride, Pyroxamine Maleate, Pyrrocaine, Pyrroliphene Hydrochloride, PyrroInitrin, Pyrvinium Pamoate, Quadazocine Mesylate, Quazepam, Quazinone, Quazodine, Quazolast, quetiapine, quiflapon, quinagolide, Quinaldine Blue, quinapril, Quinaprilat, Quinazosin Hydrochloride, Quinbolone, Quinctolate, Quindecamine Acetate, Quindonium Bromide, Quinelorane Hydrochloride, Quinestrol, Quinfamide, Quingestanol Acetate, Quingestrone, Quinidine Gluconate, Quinielorane Hydrochloride, Quinine Sulfate, Quinpirole Hydrochloride, Quinterenol Sulfate, Quinuclium Bromide, Quinupristin, Quipazine Maleate, Rabeprazole Sodium, Racephenicol, Racepinephrine, raf antagonists, Rafoxamide, Ralitoline, raloxifene, raltitrexed, ramatroban, Ramipril, Ramoplanin, ramosetron, ranelic acid, Ranimycin, Ranitidine, ranolazine, Rauwolfia Serpentina, recainam, Recainam Hydrochloride, Reclazepam, regavirumab, Regramostim, Relaxin, Relomycin, Remacemide Hydrochloride, Remifentanil Hydrochloride, Remiprostol, Remoxipride, Repirinast, Repromicin, Reproterol Hydrochloride, Reserpine, resinferatoxin, Resorcinol, retelliptine demethylated, reticulon, reviparin sodium, revizinone, rhenium Re 186 etidronate, rhizoxin, Ribaminol, Ribavirin, Riboprine, ribozymes, ricasetron, Ridogrel, Rifabutin, Rifametane, Rifamexil, Rifamide, Rifampin, Rifapentine, Rifaximin, retinamide, rilopirox, Riluzole, rimantadine, Rimcazole Hydrochloride, Rimexolone, Rimiterol Hydrobromide, rimoprogin, riodipine, Rioprostil, Ripazepam, ripisartan, Risedronate Sodium, risedronic acid, Risocaine, Risotilide Hydrochloride, rispenzepine, Risperdal, Risperidone, Ritanserin, ritipenem, Ritodrine, Ritolukast, ritonavir, rizatriptan benzoate, Rocastine Hydrochloride, Rocuronium Bromide, Rodocaine, Roflurane, Rogletimide, rohitukine, rokitamycin, Roletamicide, Rolgamidine, Rolicyprine, Rolipram, Rolitetracycline, Rolodine, Romazarit, romurtide, Ronidazole, ropinirole, Ropitoin Hydrochloride, ropivacaine, Ropizine, roquinimex, Rosaramicin, Rosoxacin, Rotoxamine, roxaitidine, Roxarsone, roxindole, roxithromycin, rubiginone Bi, ruboxyl, rufloxacin, rupatidine, Rutamycin, ruzadolane, Sabeluzole, safingol, safironil, saintopin, salbutamol, R-, Salcolex, Salethamide Maleate, Salicyl Alcohol, Salicylamide, Salicylate Meglumine, Salicylic Acid, Salmeterol, Salnacediin, Salsalate, sameridine, sampatrilat, Sancycline, sanfetrinem, Sanguinarium Chloride, Saperconazole, saprisartan, sapropterin, saquinavir, Sarafloxacin Hydrochloride, Saralasin Acetate, SarCNU, sarcophytol A, sargramostim, Sarmoxicillin, Sarpicillin, sarpogrelate, saruplase, saterinone, satigrel, satumomab pendetide, Schick Test Control, Scopafungin, Scopolamine Hydrobromide, Scrazaipine Hydrochloride, Sdi 1 mimetics, Secalciferol, Secobarbital, Seelzone, Seglitide Acetate, selegiline, Selegiline Hydrochloride, Selenium Sulfide, Selenomethionine Se 75, Selfotel, sematilide, semduramicin, semotiadil, semustine, sense oligonucleotides, Sepazonium Chloride, Seperidol Hydrochloride, Seprilose, Seproxetine Hydrochloride, Seractide Acetate, Sergolexole Maleate, Serine, Sermetacin, Sermorelin Acetate, sertaconazole, sertindole, sertraline, setiptiline, Setoperone, sevirumab, sevoflurane, sezolamide, Sibopirdine, Sibutramine Hydrochloride, signal transduction inhibitors, Silandrone, silipide, silteplase, Silver Nitrate, simendan, Simtrazene, Simvastatin, Sincalide, Sinefungin, sinitrodil, sinnabidol, sipatrigine, sirolimus, Sisomicin, Sitogluside, sizofuran, sobuzoxane, Sodium Amylosulfate, Sodium Iodide I 123, Sodium Nitroprusside, Sodium Oxybate, sodium phenylacetate, Sodium Salicylate, solverol, Solypertine Tartrate, Somalapor, Somantadine Hydrochloride, somatomedin B, somatomedin C, somatrem, somatropin, Somenopor, Somidobove, sonermin, Sorbinil, Sorivudine, sotalol, Soterenol Hydrochloride, Sparfloxacin, Sparfosate Sodium, sparfosic acid, Sparsomycin, Sparteine Sulfate, Spectinomycin Hydrochloride, spicamycin D, Spiperone, Spiradoline Mesylate, Spiramycin, Spirapril Hydrochloride, Spiraprilat, Spirogermanium Hydrochloride, Spiromustine, Spironolactone, Spiroplatin, Spiroxasone, splenopentin, spongistatin 1, Sprodiamide, squalamine, Stallimycin Hydrochloride, Stannous Pyrophosphate, Stannous Sulfur Colloid, Stanozolol, Statolon, staurosporine, stavudine, Steffimycin, Stenbolone Acetate, stepronin, Stilbazium Iodide, Stilonium Iodide, stipiamide, Stiripentol, stobadine, Streptomycin Sulfate, Streptonicozid, Streptonigrin, Streptozocin, stromelysin inhibitors, Strontium Chloride Sr 89, succibun, Succimer, Succinylcholine Chloride, Sucralfate, Sucrosofate Potassium, Sudoxicam, Sufentanil, Sufotidine, Sulazepam, Sulbactam Pivoxil, Sulconazole Nitrate, Sulfabenz, Sulfabenzamide, Sulfacetamide, Sulfacytine, Sulfadiazine, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, sulfasalazine, Sulfasomizole, Sulfazamet, Sulfinalol Hydrochloride, sulfinosine, Sulfinpyrazone, Sulfisoxazole, Sulfomyxin, Sulfonterol Hydrochloride, sulfoxamine, Sulinldac, Sulmarin, Sulnidazole, Suloctidil, Sulofenur, sulopenem, Suloxifen Oxalate, Sulpiride, Sulprostone, sultamicillin, Sulthiame, sultopride, sulukast, Sumarotene, sumatriptan, Suncillin Sodium, Suproclone, Suprofen, suradista, suramin, Surfomer, Suricamide Maleate, Suritozole, Suronacrine Maleate, Suxemerid Sulfate, swainsonine, symakalim, Symclosene, Symetine Hydrochloride, synthetic glycosaminoglycans, Taciamine Hydrochloride, Tacrine Hydrochloride, Tacrolimus, Talampicillin Hydrochloride, Taleranol, Talisomycin, tallimustine, Talmetacin, Talniflumate, Talopram Hydrochloride, Talosalate, Tametraline Hydrochloride, Tamoxifen, Tampramine Fumarate, Tamsulosin Hydrochloride, Tandamine Hydrochloride, tandospirone, tapgen, taprostene, Tasosartan, tauromustine, Taxane, Taxoid, Tazadolene Succinate, tazanolast, tazarotene, Tazifylline Hydrochloride, Tazobactam, Tazofelone, Tazolol Hydrochloride, Tebufelone, Tebuquine, Technetium Tc 99 m Bicisate, Teclozan, Tecogalan Sodium, Teecleukin, Teflurane, Tegafur, Tegretol, Teicoplanin, telenzepine, tellurapyrylium, telmesteine, telmisartan, telomerase inhibitors, Teloxantrone Hydrochloride, Teludipine Hydrochloride, Temafloxacin Hydrochloride, Tematropium Methyl sulfate, Temazepam, Temelastine, temocapril, Temocillin, temoporfin, temozolomide, Tenidap, Teniposide, tenosal, tenoxicam, tepirindole, Tepoxalin, Teprotide, terazosin, Terbinafine, Terbutaline Sulfate, Terconazole, terfenadine, terflavoxate, terguride, Teriparatide Acetate, terlakiren, terlipressin, terodiline, Teroxalene Hydrochloride, Teroxirone, tertatolol, Tesicam, Tesimide, Testolactone, Testosterone, Tetracaine, tetrachlorodecaoxide, Tetracycline, Tetrahydrozoline Hydrochloride, Tetramisole Hydrochloride, Tetrazolast Meglumine, tetrazomine, Tetrofosmin, Tetroquinone, Tetroxoprim, Tetrydamine, thaliblastine, Thalidomide, Theofibrate, Theophylline, Thiabendazole, Thiamiprine, Thiamphenicol, Thiamylal, Thiazesim Hydrochloride, Thiazinamium Chloride, Thiethylperazine, Thimerfonate Sodium, Thimerosal, thiocoraline, thiofedrine, Thioguanine, thiomarinol, Thiopental Sodium, thioperamide, Thioridazine, Thiotepa, Thiothixene, Thiphenamil Hydrochloride, Thiphencillin Potassium, Thiram, Thozalinone, Threonine, Thrombin, thrombopoietin, thrombopoietin mimetic, thymalfasin, thymopoietin receptor agonist, thymotrinan, Thyromedan Hydrochloride, Thyroxine 1 125, Thyroxine 1 131, Tiacrilast, Tiacrilast Sodium, tiagabine, Tiamenidine, tianeptine, tiapafant, Tiapamil Hydrochloride, Tiaramide Hydrochloride, Tiazofurin, Tibenelast Sodium, Tibolone, Tibric Acid, Ticabesone Propionate, Ticarbodine, Ticarcillin Cresyl Sodium, Ticlatone, ticlopidine, Ticrynafen, tienoxolol, Tifurac Sodium, Tigemonam Dicholine, Tigestol, Tiletamine Hydrochloride, Tilidine Hydrochloride, tilisolol, tilnoprofen arbamel, Tilorone Hydrochloride, Tiludronate Disodium, tiludronic acid, Timefurone, Timobesone Acetate, Timolol, tin ethyl etiopurpurin, Tinabinol, Timidazole, Tinzaparin Sodium, Tioconazole, Tiodazosin, Tiodonium Chloride, Tioperidone Hydrochloride, Tiopinac, Tiospirone Hydrochloride, Tiotidine, tiotropium bromide, Tioxidazole, Tipentosin Hydrochloride, Tipredane, Tiprenolol Hydrochloride, Tiprinast Meglumine, Tipropidil Hydrochloride, Tiqueside, Tiquinamide Hydrochloride, tirandalydigin, Tirapazamine, tirilazad, tirofiban, tiropramide, titanocene dichloride, Tixanox, Tixocortol Pivalate, Tizanidine Hydrochloride, Tobramycin, Tocamide, Tocamphyl, Tofenacin Hydrochloride, Tolamolol, Tolazamide, Tolazoline Hydrochloride, Tolbutamide, Tolcapone, Tolciclate, Tolfamide, Tolgabide, lamotrigine, Tolimidone, Tolindate, Tolmetin, Tolnaftate, Tolpovidone 1 131, Tolpyrramide, Tolrestat, Tomelukast, Tomoxetine Hydrochloride, Tonazocine Mesylate, Topiramate, topotecan, Topotecan Hydrochloride, topsentin, Topterone, Toquizine, torasemide, toremifene, Torsemide, Tosifen, Tosufloxacin, totipotent stem cell factor, Tracazolate, trafermin, Tralonide, Tramadol Hydrochloride, Tramazoline Hydrochloride, trandolapril, Tranexamic Acid, Tranilast, Transcamide, translation inhibitors, traxanox, Trazodone Hydrochloride, Trazodone-HCL, Trebenzomine Hydrochloride, Trefentanil Hydrochloride, Treloxinate, Trepipam Maleate, Trestolone Acetate, tretinoin, Triacetin, triacetyluridine, Triafungin, Triamcinolone, Triampyzine Sulfate, Triamterene, Triazolam, Tribenoside, tricaprilin, Tricetamide, Trichlormethiazide, trichohyalin, triciribine, Tricitrates, Triclofenol piperazine, Triclofos Sodium, Triclonide, trientine, Trifenagrel, triflavin, Triflocin, Triflubazam, Triflumidate, Trifluoperazine Hydrochloride, Trifluperidol, Triflupromazine, Triflupromazine Hydrochloride, Trifluridine, Trihexyphenidyl Hydrochloride, Trilostane, Trimazosin Hydrochloride, trimegestone, Trimeprazine Tartrate, Trimethadione, Trimethaphan Camsylate, Trimethobenzamide Hydrochloride, Trimethoprim, Trimetozine, Trimetrexate, Trimipramine, Trimoprostil, Trimoxamine Hydrochloride, Triolein 1 125, Triolein 1 131, Trioxifene Mesylate, Tripamide, Tripelennamine Hydrochloride, Triprolidine Hydrochloride, Triptorelin, Trisulfapyrimidines, Troclosene Potassium, troglitazone, Trolamine, Troleandomycin, trombodipine, trometamol, Tropanserin Hydrochloride, Tropicamide, tropine ester, tropisetron, trospectomycin, trovafloxacin, trovirdine, Tryptophan, Tuberculin, Tubocurarine Chloride, Tubulozole Hydrochloride, tucarcsol, tulobuterol, turosteride, Tybamate, tylogenin, Tyropanoate Sodium, Tyrosine, Tyrothricin, tyrphostins, ubenimex, Uldazepam, Undecylenic Acid, Uracil Mustard, urapidil, Urea, Uredepa, uridine triphosphate, Urofollitropin, Urokinase, Ursodiol, valaciclovir, Valine, Valnoctamide, Valproate Sodium, Valproic Acid, valsartan, vamicamide, vanadeine, Vancomycin, vaminolol, Vapiprost Hydrochloride, Vapreotide, variolin B, Vasopressin, Vecuronium Bromide, velaresol, Velnacrine Maleate, venlafaxine, veradoline Hydrochloride, veramine, Verapamil Hydrochloride, verdins, Verilopam Hydrochloride, Verlukast, Verofylline, veroxan, verteporfin, Vesnarinone, vexibinol, Vidarabine, vigabatrin, Viloxazine Hydrochloride, Vinblastine Sulfate, vinburnine citrate, Vincofos, vinconate, Vincristine Sulfate, Vindesine, Vindesine Sulfate, Vinepidine Sulfate, Vinglycinate Sulfate, Vinleurosine Sulfate, vinorelbine, vinpocetine, vintoperol, vinxaltine, Vinzolidine Sulfate, Viprostol, Virginiamycin, Viridofulvin, Viroxime, vitaxin, Volazocine, voriconazole, vorozole, voxergolide, Warfarin Sodium, Xamoterol, Xanomeline, Xanoxate Sodium, Xanthinol Niacinate, xemilofiban, Xenalipin, Xenbucin, Xilobam, ximoprofen, Xipamide, Xorphanol Mesylate, Xylamidine Tosylate, Xylazine Hydrochloride, Xylometazoline Hydrochloride, Xylose, yangambin, zabicipril, zacopride, zafirlukast, Zalcitabine, zaleplon, zalospirone, Zaltidine Hydrochloride, zaltoprofen, zanamivir, zankiren, zanoterone, Zantac, Zarirlukast, zatebradine, zatosetron, Zatosetron Maleate, zenarestat, Zenazocine Mesylate, Zeniplatin, Zeranol, Zidometacin, Zidovudine, zifrosilone, Zilantel, zilascorb, zileuton, Zimeldine Hydrochloride, Zinc Undecylenate, Zindotrine, Zinoconazole Hydrochloride, Zinostatin, Zinterol Hydrochloride, Zinviroxime, ziprasidone, Zobolt, Zofenopril Calcium, Zofenoprilat, Zolamine Hydrochloride, Zolazepam Hydrochloride, zoledronie acid, Zolertine Hydrochloride, zolmitriptan, zolpidem, Zomepirac Sodium, Zometapine, Zoniclezole Hydrochloride, Zonisamide, zopiclone, Zopolrestat, Zorbamyciin, Zorubicin Hydrochloride, zotepine, Zucapsaicin, JTT-501 (PNU-182716) (Reglitazar), AR-H039122, MCC-555 (Netoglitazone), AR-H049020, Tesaglitazar), CS-011 (CI-1037), GW-409544X, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (Ragaglitazar), L-686398, L-168049, L-805645, L-054852, Demethyl asteriquinone B1 (L-783281), L-363586, KRP-297, P32/98, CRE-16336, EML-1625, pharmaceutically acceptable salts thereof, or a biologically active fragment, variant or derivative thereof, or a combination thereof. In some embodiments, a biologically active agent is selected from: leuprolide, octreotide, brimonidine, latanoprost, latanoprost acid, travoprost, travoprost acid, brinzolamide, dorzolamide, betaxolol, terbinafine, risperidone, and/or rapamycin, or a combination thereof.

Carbohydrate: The term “carbohydrate”, as used herein, refers to a biological molecule comprising carbon, oxygen and hydrogen; in some embodiments, a carbohydrate includes a saccharide, a sugar, a starch or cellulose. In some embodiments, saccharides include monosaccharides, disaccharides, oligosaccharides and polysaccharides. In some embodiments, a polysaccharide acts as a structural component or for energy storage. In some embodiments, a carbohydrate is involved in the immune system, fertilization, preventing pathogenesis, blood clotthing and/or development. In some embodiments, a biologically active agent comprises a carbohydrate.

Cellpenetrating peptide: The terms “cell penetrating peptide”, “cell penetrating protein”, “CPP” and the like, as used herein, refer to a peptide or protein having an ability to pass through cellular membranes. In various embodiments, a CPP is conjugated to a biologically active agent to facilitate transport of the agent across the membrane. In some embodiments, the CPP is useful in facilitating the uptake of such agents across cell membranes, such as the plasma membrane of a mammalian cell and/or the nuclear membrane of a mammalian cell. In some embodiments, a CPP is capable of being internalized into a cell and passing cellular membranes (including, inter alia, the outer “limiting” cell membrane (also commonly referred to as “plasma membrane”), endosomal membranes, and membranes of the endoplasmatic reticulum) and/or directing the passage of a given agent or cargo through these cellular membranes. In some embodiments, any possible mechanism of internalization is envisaged including both energy-dependent (i.e. active) transport mechanisms (e.g., endocytosis) and energy-independent (i.e. passive) transport mechanism (e.g., diffusion). In various embodiments, internalization includes involving the localization of at least a part of the peptides that passed through the plasma cellular membrane into the cytoplasma (in contrast to localization in different cellular compartments such as vesicles, endosomes or in the nucleus). A non-limiting example of a CPP is a peptide having amino acid sequence GRKKRRQRRRPPQ (SEQ ID NO: 2) (Vives; E. et al. (1997), supra). Non-limiting examples of CPPs include the HIV-1 TAT translocation domain (Green; M. and Loewenstein, P. M. (1988) Cell 55, 1179-1188) and the homeodomain of the Antennapedia protein from Drosophila (Joliot; A. et al. (1991) Proc. Natl. Acad. Sci. USA 88, 1864-1868); a sequence of 16 amino acids called penetratin or pAntp of the Antennapedia protein (Derossi, D. et al. (1994) J. Biol. Chem. 269, 10444-10450); a basic sequence of the HIV-1 Tat protein (Vives, E. et al. (1997) J. Biol. Chem. 272, 16010-16017); and a synthetic peptide developed is the amphipathic model peptide MAP (Oehlke, J. et al. (1998) Biochim. Biophys. Acta 1414, 127-139). Additional non-limiting examples of CPPs are described in U.S. Pat. Nos. 9,303,076; and 9,302,014.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, 10, 15, 20 or more amino acids are required to be characteristic of a protein. In general, a characteristic portion is one that, in addition to the sequence identity specified above, shares at least one functional characteristic with the relevant intact protein.

Characteristic sequence: A “characteristic sequence”, as used herein, is a sequence that is found in all members of a family of polypeptides or nucleic acids, and therefore can be used by those of ordinary skill in the art to define members of the family.

Characteristic structural element: The term “characteristic structural element”, as used herein, refers to a distinctive structural element (e.g., core structure, collection of pendant moieties, sequence element, etc) that is found in all members of a family of polypeptides, small molecules, or nucleic acids, and therefore can be used by those of ordinary skill in the art to define members of the family.

Chemotherapeutic agent: The term “chemotherapeutic agent”, as used herein, refers to a drug or agent capable of killing growing cells, including cancer cells. Chemotherapeutic agents are frequently used to treat various forms of cancer. In some embodiments, non-limiting examples of chemotherapeutic agents include adriamycin, paclitaxel (Taxol), docetaxel (Taxotere), actinomycin D, doxorubicin, daunorubicin, valrubicin, idarubicin, epirubicin, bleomycin, plicamycin, camptothecin and derivatives, bleomycin, etoposide, teniposide, mitomycin, vinca alkaloids, such as vinblastine and vincristine, and platinum-based compounds such as cisplatin, gemcitabine. In some embodiments, a composition comprises a lipid and a portion of a chemotherapeutic agent capable of mediating at least one function of a chemotherapeutic agent.

Comparable: The term “comparable”, as used herein, is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Conjugate: The term “conjugate”, as used herein, refers to a composition comprising two or more components, moieties or molecules which are physically linked together, e.g., by a covalent bond, either directly or indirectly (as a non-limiting example, with one or more linkers interposed between two adjacent components, moieties or molecules). The term “conjugated”, as used herein, in reference to a composition comprising two or more components, moieties or molecules, references the state the two or more components, moieties or molecules are physically linked together. In some embodiments, a composition comprises a lipid and a biologically active agent, wherein the lipid and the biologically active agent are conjugated.

CRISPR and related terms: The term “CRISPR”, “CRISPR/Cas system” and the like, as used herein, refers to a biologically active system involving clustered regularly-interspaced short palindromic repeats (CRISPR), which are segments of prokaryotic DNA containing short repetitions of base sequences, or various artificial systems derived or inspired by the naturally-occurring prokaryotic system. In some embodiments, a biologically active agent comprises a component of a CRISPR/Cas system. In some embodiments, a component of a CRISPR/Cas system include, without limitation: a gene encoding a Cas protein (including, as non-limiting examples, Cas9, dCas9, and variants thereof, both naturally-occurring and artificial) or the protein itself, a guide RNA; any component of a CAS crRNA complex; a cas (CRISPR-associated) gene or gene product; and any other biologically active molecule involved in a naturally-occurring or artificial CRISPR/Cas system. See, for example, Jinek et al. 2012 Science 337: 816-821; Cong et al. 2013 Science 339: 819-823; U.S. Pat. App. 20140234972; DiCarlo 2013 Nucl. Acids Res. 41: 4336-43; Hwang et al. 2013 Nat. Biotech. 31: 227-9; and Flowers et al. 2014 Development 141: 2165-71.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₅-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Equivalent agents: Those of ordinary skill in the art, reading the present disclosure, will appreciate that the scope of useful agents in the context of the present invention is not limited to those specifically mentioned or exemplified herein. In particular, those skilled in the art will recognize that active agents typically have a structure that consists of a core and attached pendant moieties, and furthermore will appreciate that simple variations of such core and/or pendant moieties may not significantly alter activity of the agent. For example, in some embodiments, substitution of one or more pendant moieties with groups of comparable three-dimensional structure and/or chemical reactivity characteristics may generate a substituted compound or portion equivalent to a parent reference compound or portion. In some embodiments, addition or removal of one or more pendant moieties may generate a substituted compound equivalent to a parent reference compound. In some embodiments, alteration of core structure, for example by addition or removal of a small number of bonds (typically not more than 5, 4, 3, 2, or 1 bonds, and often only a single bond) may generate a substituted compound equivalent to a parent reference compound. In many embodiments, equivalent compounds may be prepared by methods illustrated in general reaction schemes as, for example, described below, or by modifications thereof, using readily available starting materials, reagents and conventional or provided synthesis procedures. In these reactions, it is also possible to make use of variants, which are in themselves known, but are not mentioned here.

Equivalent Dosage: The term “equivalent dosage”, as used herein, is used herein to compare dosages of different pharmaceutically active agents that effect the same biological result. Dosages of two different agents are considered to be “equivalent” to one another in accordance with the present invention if they achieve a comparable level or extent of the biological result. In some embodiments, equivalent dosages of different pharmaceutical agents for use in accordance with the present invention are determined using in vitro and/or in vivo assays as described herein. In some embodiments, one or more lysosomal activating agents for use in accordance with the present invention is utilized at a dose equivalent to a dose of a reference lysosomal activating agent; in some such embodiments, the reference lysosomal activating agent for such purpose is selected from the group consisting of small molecule allosteric activators (e.g., pyrazolpyrimidines), imminosugars (e.g., isofagomine), antioxidants (e.g., n-acetyl-cysteine), and regulators of cellular trafficking (e.g., Rabla polypeptide).

Halogen: The term “halogen”, as used herein, means F, Cl, Br, or I.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, or CH₃ are independently replaced by one or more heteroatoms (including oxidized and/or substituted form thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.).

Heterocyclyl: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Immunomodulatory nucleic acid and CpG oligonucleotide and related terms: The term “immunomodulatory nucleic acid”, as used herein, refers to a nucleic acid which is capable of modulating an immune response, e.g., in a mammal, e.g., in a human subject. In various embodiments, the immunomodulatory nucleic acid is capable of stimulating (agonizing) an immune response; in other embodiments, different immunomodulatory nucleic acids are capable of decreasing (antagonizing) an immune response. In non-limiting examples, an immunomodulatory nucleic acid includes a CpG oligonucleotide. The term “CpG oligonucleotide”, as used herein, refers to an oligonucleotide comprising an unmethylated CpG motif, wherein the oligonucleotide can comprise nucleotides, modified nucleotides and/or nucleotide analogs. In some embodiments, a CpG oligonucleotide is capable of agonizing a TLR9-mediated and/or TLR9-associated immune response in at least one assay; in some embodiments, a CpG oligonucleotide is capable of antagonizing an immune response in at least one assay. Others do neither. In some embodiments, a CpG oligonucleotide can optionally comprise modifications of the sugar, base or phosphate (phosphodiester), as well as secondary and tertiary structures. See, for example, Vollmer et al. 2009 Adv. Drug. Del. Rev. 61: 195-204. In some embodiments, an example of a modified phosphodiester is a phosphorothioate. In some embodiments, one or more phosphorothioates (PS) is incorporated into the backbone of a CpG oligonucleotide (in place of a phosphodiester or PO); the PS can reportedly reduce nuclease degradation and, in at least some cases, enhance the immunogenic activity of the CpG oligonucleotide 10- to 100-fold. Vollmer et al. 2009 Adv. Drug Del. Rev. 61: 195-204. In some embodiments, a CpG oligonucleotide can comprise all phosphodiesters in the backbone; or a mixture of phosphodiesters and internucleoside linkers in the backbone; or all internucleoside linkers in the backbone. For example, WO 2015/108047 reports CpG oligonucleotides with a mixture of phosphodiester and internucleoside (e.g., phosphorothioate) linkages; in this case, the CpG region motif comprises phosphodiesters, with phosphorothioates flanking the CpG region motif. In various embodiments, the CpG oligonucleotide can comprise a phosphorothioate which is in the Rp or Sp conformation. The terms “CpG ODN” or “CpG oligodeoxynucleotide” as used in the literature, and as used herein, are not strictly limited to oligonucleotides wherein “p” is a phosphate; these terms have previously been used in the literature and are used herein to encompass oligonucleotides which comprise one or more phosphorothioates in place of phosphodiesters, or even comprise all phosphorothioates in their backbones, and/or other modifications. In some embodiments, an “immunostimulatory” CpG oligonucleotide is capable of agonizing an immune response. In some embodiments, a CpG oligonucleotide can comprise one strand; or, optionally, it can further comprise a second or other additional strands. In some embodiments, a CpG oligonucleotide can further comprise or be conjugated to other components which are not nucleotides. In some embodiments, a composition comprises a lipid and a portion of an immunomodulatory nucleic acid capable of mediating at least one function of an immunomodulatory nucleic acid.

Intraperitoneal: The phrases “intraperitoneal administration” and “administered intraperitonealy” as used herein have their art-understood meaning referring to administration of a compound or composition into the peritoneum of a subject.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

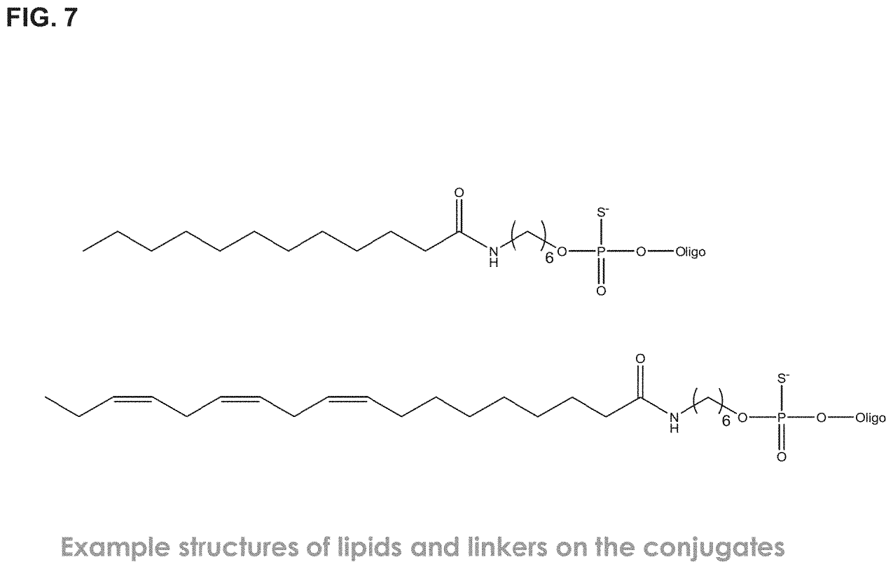

Linker: The term “linker”, as used herein, refers to a moiety that connects two parts of a composition; as a non-limiting example, a linker physically connects a biologically active agent to a lipid. Non-limiting examples of suitable linkers include: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group. Other non-limiting examples of linkers are described herein, or detailed in FIG. 7.

Lower alkyl: The term “lower alkyl”, as used herein, refers to a C₁₋₄ straight or branched alkyl group. Example lower alkyl groups are methyl, ethyl, propyl, isopropyl, butyl, isobutyl, and tert-butyl.

Lipid: The term “lipid”, as used herein, refers to any member of a large group of molecules which are generally at least partially hydrophobic or amphiphilic, and include, inter alia, phospholipids, triglycerides, diglycerides, monoglycerides, fat-soluble vitamins, sterols, fats and waxes. In some embodiments, lipids include fatty acids, glycerolipids, glycerophospholipids, sphingolipids, sterol lipids, prenol lipids, saccharolipids, polyketides, and other molecules. In some embodiments, the present disclosure pertains to a composition comprising a biologically active agent and a lipid comprising a C₁₀-C₈₀ linear, saturated or partially unsaturated, aliphatic chain. In some embodiments, the present disclosure pertains to a composition comprising a biologically active agent and a lipid comprising a C₁₀-C₈₀ linear, saturated or partially unsaturated, aliphatic chain, optionally substituted with one or more C₁₋₄ aliphatic group. In some embodiments, the present disclosure pertains to a composition comprising a biologically active agent and a lipid comprising a C₁₀-C₆₀ linear, saturated or partially unsaturated, aliphatic chain. In some embodiments, the present disclosure pertains to a composition comprising a biologically active agent and a lipid comprising a C₁₀-C₆₀ linear, saturated or partially unsaturated, aliphatic chain, optionally substituted with one or more C₁₋₄ aliphatic group. In some embodiments, the present disclosure pertains to a composition comprising a biologically active agent and a lipid comprising a C₁₀-C₄₀ linear, saturated or partially unsaturated, aliphatic chain. In some embodiments, the present disclosure pertains to a composition comprising a biologically active agent and a lipid comprising a C₁₀-C₄₀ linear, saturated or partially unsaturated, aliphatic chain, optionally substituted with one or more C₁₋₄ aliphatic group. In some embodiments, a lipid includes, without limitation, lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, a lipid includes, without limitation: an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; and a targeting lipid; and any other lipid described herein or reported in the art. In some embodiments, a composition comprises a lipid and a portion of another lipid capable of mediating at least one function of another lipid. In various embodiments, a composition of the present disclosure comprises any one or more of any lipid described herein or known in the art.

lncRNA: The terms “Long non-coding RNA” and “lncRNA”, as used herein, refer to non-protein coding RNA transcripts longer than about 200 nucleotides. This numerical limit distinguishes long ncRNAs from small regulatory RNAs such as microRNAs (miRNAs), short interfering RNAs (siRNAs), Piwi-interacting RNAs (piRNAs), small nucleolar RNAs (snoRNAs), and other short RNAs. In some embodiments, a lncRNA bears one or more signatures of mRNAs, including 5′ capping, splicing, and poly-adenylation, but has little or no open reading frame (ORF). In some embodiments, a lncRNA is Air or Xist. In some embodiments, a lncRNA functions in regulating expression of another gene. In some embodiments, a lncRNA is a lncRNA listed in any lncRNA database, including, but not limited to: ChIPBase, C-It-Loci, LNCipedia, lncRNABase, lncRNAdb, lncRNome, MONOCLdb, NONCODE, and NRED. In some embodiments, a composition comprises a lipid and a portion of a lncRNA capable of mediating at least one function of a lncRNA.

mRNA: The terms “Messenger RNA”, “mRNA” and the like, as used herein, refer to any of a large family of RNA molecules that convey genetic information from DNA to the ribosome, where they specify the amino acid sequence of the protein products of gene expression. In various embodiments, following transcription of primary transcript mRNA (known as pre-mRNA) by RNA polymerase, processed, mature mRNA is translated into a polymer of amino acids: a protein, as summarized in the central dogma of molecular biology. In some embodiments, the mRNA includes a modified mRNA or mmRNA. U.S. Pat. No. 9,220,792. In some embodiments, a mRNA encodes any of: an allergen, a blood component, a gene therapy product, a human tissue or cellular product used in transplantation, a vaccine, an antibody, a cytokine, a growth factor, an enzyme, a thrombolytic, or an immunomodulator. In some embodiments, a composition comprises a lipid and a portion of a mRNA capable of mediating at least one function of a mRNA.

Muscle: The term “muscle”, as used herein, refers to a type of tissue found in animals (including, without limitation, mammals, including humans); muscle tissue is a type of fibrous tissue that has the ability to contract, producing movement in or maintaining the position of parts of the body. A muscle cell or tissue includes any skeletal muscle cell or tissue, cardiac muscle cell or tissue, smooth muscle cell or tissue, and/or myoepithelial cell or tissue. In some embodiments, a muscle cell or tissue includes a heart muscle cell or tissue. In some embodiments, a muscle cell or tissue includes a thoracic diaphragm muscle cell or tissue. In some embodiments, a muscle cell or tissue is a skeletal muscle cell or tissue. In various embodiments, a muscle cell or tissue is selected from: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, aryjordanicus, auricularis, biceps brachii, biceps femoris, brachialis, brachioradialis, buccinator, bulbospongiosus, constrictor of pharynx—inferior, constrictor of pharynx—middle, constrictor of pharynx—superior, coracobrachialis, corrugator supercilii, cremaster, cricothyroid, dartos, deep transverse perinei, deltoid, depressor anguli oris, depressor labii inferioris, thoracic diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei—dorsal of hand, interossei—dorsal of foot, interossei—palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani—coccygeus, levator ani—iliococcygeus, levator ani—pubococcygeus, levator ani—puborectalis, levator ani—pubovaginalis, levator labii superioris, levator labii superioris, alaeque nasi, levator palpebrae superioris, levator scapulae, levator veli palatini, levatores costarum, longus capitis, longus colli, lumbricals of foot (4), lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse, perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis—multifidus, transversospinalis—rotatores, transversospinalis—semispinalis, transversus abdominis, transversus thoracis, trapezius, triceps, vastus intermedius, vastus lateralis, vastus medialis, zygomaticus major, and zygomaticus minor. In some embodiments, the muscle cell or tissue is a smooth muscle cell or tissue. In various embodiments, the muscle cell or tissue is selected from a muscle cell or tissue found in any of: esophagus, stomach, intestines, bronchi, uterus, urethra, bladder, blood vessels, and the arrector pili in the skin. In various embodiments, a muscle cell or tissues includes any structure or sub-structure which is a part of a muscle, including, but not limited to: epimysium, myocyte, sarcomere, tendon, fascile, muscle fiber, perimysium, collagen, collagen fiber, muscle spindle, sarcolemma, sarcoplasmic reticulum, thin filament, thick filament, Z disc, H zone, I band, A band or M line. In some embodiments, the muscle cell or tissue is healthy. In some embodiments, the muscle cell or tissue is afflicted with a disorder or disease.

Muscle-related disorder and the like: The terms “muscle-related disorder”, “muscle-related disease” and the like, as used herein, refers to a disease or disorder associated with a muscle cell or tissue, or neuromuscular system, including a skeletal muscle cell or tissue, cardiac muscle cell or tissue, smooth muscle cell or tissue, or myoepithelial cell or tissue, or other muscle cell or tissue. In various embodiments, the present disclosure pertains to a method pertaining to a composition comprising a lipid and a biologically active agent, wherein the composition is administered to a subject who is suffering from a muscle-related disorder. In various embodiments, a muscle-related disorder is sarcopenia, a muscle movement disorder, a muscle wasting-related disorder, muscle degeneration, muscle weakness, muscular dystrophy, Duchenne muscular dystrophy, heart failure, breathing disorder, skeletal muscle degeneration caused by malnutrition and disease, a muscle-related disease related to impaired insulin-dependent signaling, amyotrophic lateral sclerosis, spinal muscle atrophy and spinal cord injury, ischemic muscle disease. In some embodiments, a muscle related disorder includes, for example, shoulder stiffness, frozen shoulder (stiff shoulder due to age), rheumatoid arthritis, myofascitis, neck muscle rigidity, neck-shoulder-arm syndrome, whiplash syndrome, sprain, tendon sheath inflammation, low back pain syndrome, skeletal muscle atrophy and the like. In some embodiments, a muscle movement disorder includes a condition associated with one or more of bruxism, periodic limb movement disorder, restless leg syndrome, muscular dystrophy, muscle inflammation, pinched nerves, peripheral nerve damage, amyotrophic lateral sclerosis, myasthenia gravis, and disc herniation, sleep-related involuntary muscle movement disorder. In some embodiments, a muscle wasting-related disorder is a disease or condition that involves symptoms such as the gradual loss of muscle mass. In some embodiments, a muscle wasting is attributed to any of various causes, including genetic predispositions; age-related diseases such as hypertension, impaired glucose tolerance, diabetes, obesity, dyslipidemia, atherosclerosis, and cardiovascular diseases; chronic diseases such as cancers, autoimmune diseases, infectious diseases, AIDS, chronic inflammatory diseases, arthritis, malnutrition, renal diseases, chronic obstructive pulmonary disease, pulmonary emphysema, rachitis, chronic lower spine pain, peripheral nerve injury, central nerve injury, and chemical injury; conditions such as long-term immobilization, ineffectualness-like conditions such as bone fracture or trauma, and post-surgery bed rest; and the progressive decrease in skeletal muscle mass and strength caused by aging processes. The muscle wasting-related disease can cause weakened physical conditions, which can deteriorate health conditions and induce incapable physical activity. In some embodiments, sarcopenia is the gradual decrease in skeletal muscle mass caused by aging, which can directly cause a decrease in muscle strength, resulting in a decrease and impairment in various physical functions. In some embodiments, a muscular dystrophy is a disorder in which strength and muscle bulk gradually decline. Non-limiting examples of muscular dystrophy diseases includes Becker muscular dystrophy, tibial muscular dystrophy, Duchenne muscular dystrophy, Emery-Dreifuss muscular dystrophy, facioscapulohumeral muscular dystrophy, sarcoglycanopathies, congenital muscular dystrophy such as congenital muscular dystrophy due to partial LAMA2 deficiency, merosin-deficient congenital muscular dystrophy, type 1D congenital muscular dystrophy, Fukuyama congenital muscular dystrophy, limb-girdle type 1A muscular dystrophy, limb-girdle type 2A muscular dystrophy, limb-girdle type 2B muscular dystrophy, limb-girdle type 2C muscular dystrophy, limb-girdle type 2D muscular dystrophy, limb-girdle type 2E muscular dystrophy, limb-girdle type 2F muscular dystrophy, limb-girdle type 2G muscular dystrophy, limb-girdle type 2H muscular dystrophy, limb-girdle type 21 muscular dystrophy, limb-girdle type 21 muscular dystrophy, limb-girdle type 2J muscular dystrophy, limb-girdle type 2K muscular dystrophy, limb-girdle type IC muscular dystrophy, rigid spine muscular dystrophy with epidermolysis bullosa simplex, oculopharyngeal muscular dystrophy, Ullrich congenital muscular dystrophy, and Ullrich scleroatonic muscular dystrophy. In some embodiments, a subject has Duchenne muscular dystrophy. In some embodiments, a muscle degeneration is caused by an injury, by a degenerative muscle disease or disorder, or by a disease, disorder or damage to the nervous system which results in denervation of muscle. Such diseases or disorders include, but are not limited to, degenerative or inflammatory muscle diseases such as muscular dystrophy, myotonic dystrophy, fascio-scapulo-humoral dystrophy, limb girdle dystrophy, distal muscular dystrophy or myositis or peripheral neuropathies associated with diabetic neuropathy, acute neurapraxia, neurotmesis or axotmesis. In addition, the methods described herein can be used to diagnose or monitor neurological degenerative diseases, especially those associated with degeneration of motor neurons, such as amylotrophic laterial sclerosis, spinal muscular atrophy, post-polio syndrome, infantile muscular atrophy, poliomyelitis or Charlot-Marie Tooth disease or inflammatory or demyelinating neurological diseases or disorders such as Guillan-Barre Syndrome or chronic inflammatory demyelinating polyneuropathy. The methods of the present invention may also be used to diagnose or monitor degeneration caused by nerve injuries such as those associated with carpal tunnel syndrome, compression, mechanical severance of a nerve or a tumor. In addition, the methods disclosed herein may be utilized to diagnose neural or non-neuronal tumors.

ncRNA: The term “ncRNA”, as used herein, refers to non-coding RNA, of which there are several types, including, but not limited to lncRNA (long non-coding RNA). In some embodiments, a ncRNA participates in regulating the expression of a gene or protein or gene product. Wahlestedt 2013 Nat. Rev. Drug Disc. 12: 433-446. Antagonists to ncRNAs have been reported. Meng et al. 2015 Nature 518: 409-412; and Ling et al. 2013 Nature Rev. Drug Discov. 12: 847-865. In some embodiments, a composition comprises a biologically active agent and a lipid, wherein the biologically active agent is a nucleic acid or other antagonist to a ncRNA. In some embodiments, a composition comprises a lipid and a portion of a ncRNA capable of mediating at least one function of a ncRNA.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide”, as used herein, refers to a molecule comprising a plurality of amino acids joined together via peptide bonds. In some embodiments, a peptide includes a dipeptide, tripeptide, oligopeptide and polypeptide. In some embodiments, a dipeptide contains two amino acids; a tripeptide contains three amino acids; and an oligopeptide comprises about 2 to about 50 or more amino acids. In some embodiments, peptides comprise more than about 50 amino acids. In some embodiments, a polypeptide and a protein are also molecules comprising a plurality of amino acids joined together via peptide bonds. In some embodiments, a peptide includes any therapeutic peptide listed in the SATPdb database of therapeutic peptides. Singh et al. 2015 Nucl. Acids Res. doi: 10.1093/nar/gkv1114. In some embodiments, a composition comprises a lipid and a portion of a peptide capable of mediating at least one function of a peptide.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Plasmid: The term “plasmid”, as used herein, refers to an extra-chromosomal (apart from a chromosome) length of DNA; plasmids are generally circular and generally capable of independent replication, though exceptions exist such as linear plasmids and plasmids which are not capable of independent replication (including, but not limited to, suicide vectors). In some embodiments, a plasmid can be extra-chromosomal under some conditions (e.g., in a laboratory), but capable of integrating into a chromosome (e.g., acting as a suicide vector capable of integrating into a chromosome in a cell or subject). Plasmids naturally exist in many organisms, including bacteria and some eukaryotic organisms, and are commonly engineered and produced artificially to carry genes into an organism. A plasmid is generally double-stranded, or can alternatively be single-stranded or partially single- and double-stranded, or have other strandedness. Artificial plasmids are commonly used in genetic engineering. Plasmids include plasmids encoding or capable of expressing a nucleic acid, including, without limitation, a mRNA, a RNAi agent or precursor thereof, an antagonist to another nucleic acid (including, without limitation, an antagonist to a miRNA, RNAi agent, mRNA, etc.) or precursor thereof, or other nucleic acids of therapeutic benefit. Additional parts of a plasmid can optionally include one or more copies of any one or more component selected from: a gene encoding a protein related to replication, an origin or replication, a gene encoding a replication initiator protein, an origin of replication enhancer, a gene encoding a nucleic acid of therapeutic benefit (or a precursor thereof), one or multiple promoters, one or multiple transcription enhancers, one or multiple transcription terminators, one or more marker genes (e.g., a gene encoding resistance to an antibiotic or encoding an enzyme required for survival and/or growth under certain laboratory conditions). In some embodiments, a plasmid is a suicide vector, which can lack any of: an origin of replication, a gene encoding a DNA replication initiator protein, or any other component required for independent replication. In some embodiments, two plasmids can be physically separate, but produce products which work in concert; for example, one plasmid can encode a gene for a transcriptional enhancer which enhances transcription of a gene encoded on another plasmid; for another example, one plasmid can comprise a gene encoding a DNA replication initiator protein which initiates replication at a DNA replication origin on another plasmid. Various plasmids are known in the art. In some embodiments, a composition comprises a lipid and a portion of a plasmid capable of mediating at least one function of a plasmid.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O— nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group.

In some embodiments, a phosphorous protecting group is a group attached to the internucleotide phosphorous linkage throughout oligonucleotide synthesis. In some embodiments, the phosphorous protecting group is attached to the sulfur atom of the internucleotide phosphorothioate linkage. In some embodiments, the phosphorous protecting group is attached to the oxygen atom of the internucleotide phosphorothioate linkage. In some embodiments, the phosphorous protecting group is attached to the oxygen atom of the internucleotide phosphate linkage. In some embodiments the phosphorous protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, 4-[N-methyl-N-(2,2,2-trifluoroacetyl)amino]butyl.

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contain a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both; in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Ribozymes: The term “ribozyme”, as used herein, refers to a catalytic RNA that functions as an enzyme and does not require proteins for catalysis. In some embodiments, a ribozyme is a self-processing RNA that catalyzes RNA cleavage and ligation reactions. In some embodiments, a substrate recognition domain of a ribozyme is artificially engineered to stimulate site-specific cleavage in cis (the same nucleic acid strand) or trans (a non-covalently linked nucleic acid). Scherer et al. 2003 Nat Biotechnol. 21:1457-1465. In some embodiments, a ribozyme is subject to in vitro selection and directed evolution to generate improved properties and new functions for therapeutic and diagnostic reagents. In some embodiments, a ribozyme is engineered to be allosterically activated by effector molecules, which has led to the development of artificial “riboswitches” as biosensors and synthetic biological tools. Wieland et al. 2010 Chem Biol. 17:236-242; Liang et al. 2011 Mol Cell. 43:915-926. In some embodiments, a ribozyme is derived from a “hammerhead” or “hairpin/paperclip” motifs. In some embodiments, a ribozyme is delivered to the target cells in RNA form or can be transcribed from therapeutic genes. In some embodiments, a ribozyme is chemically modified with any one or more of the following modifications: 5′-PS backbone linkage, 2′-O-Me, 2′-deoxy-2′-C-allyl uridine, and terminal inverted 3′-3′ deoxyabasic nucleotides. A non-limiting example of a ribozyme is Angiozyme (RPI.4610), which targets the mRNA of the vascular endothelial growth factor receptor-1 (VEGFR-1) to block angiogenesis and tumor growth. Kobayashi et al. 2005 Cancer Chemother Pharmacol. 56:329-336; Weng et al. 2005 Mol Cancer Ther. 4:948-955. Another non-limiting example of a ribozyme is Heptazyme, a synthetic ribozyme against hepatitis C virus (HCV). Sandberg et al. 2001 Hepatology 34:333a-333a; Tong et al. 2002 Hepatology 36:360a-360a; Berk 2006 Hepatology 43:S13-S30. In some embodiments, Ribozymes include those that target any of: VEGFR-1, HCV IRES, HIV U5 and pol, HIV Tat and Vpr, CCR5, HIV Tat and Rev. In some embodiments, a composition comprises a lipid and a portion of a ribozyme capable of mediating at least one function of a ribozyme.

RNAi agent: The term “RNAi agent”, as used herein, refers to a molecule capable of mediating RNA interference. The term encompasses a variety of strucures and formats, including, as a non-limiting example, siRNAs (including but not limited to those of the “canonical” structure), in addition to various natural and artificial structures capable of mediating RNA interference. The term “RNA interference” or “RNAi”, as used herein, refers to a post-transcriptional, targeted gene-silencing technique that uses a RNAi agent to degrade messenger RNA (mRNA) containing a sequence which is the same as or very similar to the RNAi agent. See: Zamore and Haley, 2005, Science, 309, 1519-1524; Zamore et al., 2000, Cell, 101, 25-33; Elbashir et al., 2001, Nature, 41 1, 494-498; and Kreutzer et al., PCT Publication WO 00/44895; Fire, PCT Publication WO 99/32619; Mello and Fire, PCT Publication WO 01/29058; and the like. The process of RNAi occurs naturally when long dsRNA is introduced into a cell and cleaved by ribonuclease III (Dicer) into shorter fragments called siRNAs. Naturally produced siRNAs are typically about 21 nucleotides long and comprise about 19 base pair duplexes with two 2-nt overhangs (the “canonical” structure). One strand of the siRNA is reportedly incorporated into the RNA-induced silencing complex (RISC). This strand (known as the anti-sense or guide strand strand) guides RISC to a complementary mRNA. One or more nucleases in the RISC then reportedly mediates cleavage of the target mRNA to induce silencing. Cleavage of the target RNA reportedly takes place in the middle of the region complementary to the anti-sense strand. See: Nykanen, et al. 2001 Cell 107:309; Sharp et al. 2001 Genes Dev. 15:485; Bernstein, et al. 2001 Nature 409:363; Elbashir, et al. 2001 Genes Dev. 15:188. As various non-limiting examples, a RNAi agent includes: siRNAs (including but not limited to those of the canonical structure), shRNAs, miRNAs, sisiRNAs, meroduplex RNAs (mdRNAs), DNA-RNA chimeras, siRNAs comprising two mismatches (or more mismatches), neutral siRNAs, aiRNAs, or a siRNA comprising a terminal or internal spacer (e.g., an 18-mer format siRNA). In various non-limiting examples, the RNAi agent is a shRNA (small hairpin RNA or short hairpin RNA), which reportedly comprises a sequence of RNA that makes a tight hairpin turn and, like siRNAs, silences targets via RISC. The antisense and sense strand are thus reportedly connected by a hairpin. shRNAs reportedly can be expressed, for example, via delivery of plasmids or through viral or bacterial vectors. Various varieties of shRNAs have been reported in the art. See, for example: Xiang et al. 2006. Nature Biotech. 24: 697-702; Macrae et al. 2006 Science 31 1: 195-8. Lombardo et al. 2007. Nature Biotech. 25: 1298-1306; Wang et al. 2011. Pharm. Res. 28: 2983-2995; Senzer et al. 2011 Mol. Ther. 20: 679-686. In various non-limiting examples, the RNAi agent is a miRNA (microRNA), which reportedly is a small RNA molecule (ca. 22 nt) that, like siRNAs, also silences targets via RISC. Naturally-occurring miRNAs are encoded by eukaryotic nuclear DNA; miRNAs are generated by post-transcriptional RNA processing, and function via base-pairing with complementary sequences within mRNA molecules, usually resulting in translational repression or target degradation and gene silencing. The human genome can reportedly encode over 1000 miRNAs, which may target about 60% of mammalian genes and are abundant in many human cell types. Various varieties of naturally-occurring and artificial derivatives of miRNAs have been reported in the art. See, for example: Lewis et al. 2003. Cell 1 15: 787-798; Lim et al. 2003. Genes Dev. 17: 991-1008; He et al. 2004. Nat. Rev. Genet. 5: 522-31; Bentwich et al. 2005. Nat. Genet. 37: 766-70; Lewis et al. 2005. Cell 120: 15-20; Kusenda et al. 2006. Biomed Pap Med Fac Univ Palacky Olomouc Czech Repub 150: 205-15; Zhang et al. 2006. J. Gen. Gen. 36: 1-6; Brodersen et al. 2008. Science 320: 1 185-90; Friedman et al. 2009. Genome Res. 19 (1): 92-105; Bartel 2009. Cell 136 (2): 215-33. In various non-limiting examples, the RNAi agent is a sisiRNA (small internally segmented interfering RNA), wherein the sense strand comprises at least one single-stranded nick. This nick decreases the incorporation of the sense strand into the RISC complex and thus reduces off-target effects. See: WO 2007/107162. In various non-limiting examples, a DNA-RNA chimera, wherein the seed portion of each strand is DNA, while the remainder of each strand is RNA. See: Yamato et al. 2011 Cancer Gene Ther. 18: 587-597. In various non-limiting examples, the RNAi agent is a siRNA comprising two mismatches, wherein that the molecule reportedly comprises three short double-stranded regions. In one embodiment of this RNAi agent, the guide (antisense) strand is a 22-mer, while the sense strand is a 20-mer (producing only a single 2-nt overhang on the 3′ end of the anti-sense strand; and two mismatches reportedly produce double-stranded regions of 6, 8 and 4 bp. See: U.S. Pat. App. 2009/0209626. In various embodiments, the RNAi agent is a neutral siRNA, in which the negative charges of the phosphate backbone are reversibly masked; Meade et al. 2014 Nat. Biotech. 32: 1256-1261. In various non-limiting examples, the RNAi agent is a aiRNA (assymetrical interfering RNA) which comprises a sense strand is shorter than 19-nt long, so that the anti-sense strand is reportedly preferentially loaded into RISC, and thus off-target effects are reduced. In various embodiments of this RNAi agent, the anti-sense strand is 21-nt long, but the sense strand is only 15 or 16 nt long. See: Sun et al. 2008 Nature Biotech. 26: 1379-1382; and Chu and Rana. 2008 RNA 14: 1714-1719. In various non-limiting examples, the RNAi agent is a siRNA comprising a terminal or internal spacer (e.g., an 18-mer format siRNA), which reportedly comprises a strand which is shorter than that of a canonical siRNA, wherein the strand comprises an internal or terminal spacer such as a ribitol or other type of non-nucleotidic spacer. See: WO2015/051366. In some embodiments, RNAi agents include those that target any of: miR-122, VEGF, VEGF-R1, RTP801, Caspase 2, KRT6A (N171K), ADRB2, TRPV1, Syk kinase, RSV Nucleocapsid, Beta catenin, KRASG12D, Apo B, PLK1, KSP and VEGF, TTR, Bcr-Abl, PKN3, P53, RRM2, Furin and GM-CSF, LMP2, LMP7, MECL1, HIV Tat and Rev. In some embodiments, a composition comprises a lipid and a portion of a RNAi agent capable of mediating at least one function of a RNAi agent.

Sample: A “sample” as used herein is a specific organism or material obtained therefrom. In some embodiments, a sample is a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, a sample is an organism. In some embodiments, a sample is a plant. In some embodiments, a sample is an animal. In some embodiments, a sample is a human. In some embodiments, a sample is an organism other than a human.

Small molecule: The terms “small molecule” or “low molecular weight molecule” or “LMW molecule” and the like, as used herein, refer to molecules which have a relatively low molecular weight. As a non-limiting example, small molecules include molecules that are less than about 7500, 7000, 6000, 5000, 4000, 3000, 2500, 2000, 1500, 1000, 900, 800, 700, 600, 500, 400, 300, 200, or 100 molecular weight. In some embodiments, a small molecule is a biologically active agent, and inhibit or decrease target gene or target gene product level, product, and/or activity. Example small molecules include, but are not limited to, small organic molecules (e.g., Cane et al. 1998. Science 282: 63), and natural product extract libraries. In another embodiment, small molecules are small, organic non-peptidic compounds. In some embodiments, small molecule inhibitors indirectly or directly inhibit or decrease target gene or target gene product level, product, and/or activity. In some embodiments, a composition comprises a lipid and a portion of a small molecule capable of mediating at least one function of a small molecule.

Small nucleolar RNAs (snoRNAs): The terms “small nucleolar RNA”, “snoRNA” and the like, as used herein, refer to any of a class of small RNA molecules that, for example, guide chemical modifications of other RNAs. In some embodiments, snoRNAs are capable of guiding chemical modifications of other RNAs, including ribosomal RNAs, transfer RNAs and small nuclear RNAs. In some embodiments, there are reportedly two main classes of snoRNA, the C/D box snoRNAs, which are associated with methylation, and the H/ACA box snoRNAs, which are associated with pseudouridylation.

Splice switching oligonucleotide (SSO): The term “Splice switching oligonucleotide” or “SSO”, as used herein, refers to an oligonucleotide capable of altering the splicing of a pre-mRNA. In a non-limiting example, a SSO can bind to a 5′ or 3′ splicing junction or to exonic splicing enhancer or silencing sites. In doing so, a SSO can modify splicing in various ways, such as promoting alternative use of exons, exon exclusion, or exon inclusion. In various embodiments, a SSO can cause an exon to be skipped; or, in other cases, prevent the skipping of an exon. Crooke 2004 Curr. Mol. Med. 4: 465-487; Bennett et al. 2010 Ann. Rev. Pharmacol. Toxicol. 50: 259-293; and Kole et al. 2012 Nat. Rev. Drug Discov. 11: 125-140. A non-limiting example of a SSO is an oligonucleotide which is reportedly capable of mediating skipping of an exon in dystrophin pre-mRNA. A non-limiting example of a SSO is WV-942. A non-limiting example of a SSO is an oligonucleotide which is capable of preventing the skipping of an exon in the SMN2 pre-mRNA; see Rigo et al. 2012 J. Cell Biol. 199: 21-25; and Kaczmarek et al. 2015 Exp. Opin. Exp. Drugs 24: 867-881. In some embodiments, a composition comprises a lipid and a portion of a snoRNA capable of mediating at least one function of a snoRNA. In some embodiments, a SSO switches splicing in a gene related to a muscle-related disorder. In some embodiments, a SSO is capable of skipping or mediating the skipping of an exon, wherein a mutation in the exon is related to a muscle-related disorder. In some embodiments, a SSO is capable of preventing the skipping or mediating the prevent of skipping of an exon, wherein a mutation in the exon is related to a muscle-related disorder. In some embodiments, a SSO is capable of skipping or mediates skipping of an exon in the dystrophin gene. In some embodiments, a SSO is capable of skipping or mediates skipping of exon 51, 45, 53 or 44 in the dystrophin gene. In some embodiments, a SSO is capable of preventing or mediating the prevention of skipping of an exon in a gene related to SMA. In some embodiments, a SSO is capable of preventing or mediating the prevention of skipping of an exon in the SMN2 gene. In some embodiments, a SSO is capable of preventing or mediating the prevention of skipping of exon 7 in the SMN2 gene.

Stereochemically isomericforms: The phrase “stereochemically isomeric forms,” “stereoisomers,” and the like, as used herein, refers to different compounds made up of the same atoms bonded by the same sequence of bonds but having different three-dimensional structures which are not interchangeable. In some embodiments of the invention, provided chemical compositions may be or include pure preparations of individual stereochemically isomeric forms of a compound; in some embodiments, provided chemical compositions may be or include mixtures of two or more stereochemically isomeric forms of the compound. In certain embodiments, such mixtures contain equal amounts of different stereochemically isomeric forms; in certain embodiments, such mixtures contain different amounts of at least two different stereochemically isomeric forms. In some embodiments, a chemical composition may contain all diastereomers and/or enantiomers of the compound. In some embodiments, a chemical composition may contain less than all diastereomers and/or enantiomers of a compound. In some embodiments, if a particular enantiomer of a compound of the present invention is desired, it may be prepared, for example, by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, diastereomeric salts are formed with an appropriate optically-active acid, and resolved, for example, by fractional crystallization. In some embodiments, a composition which is stereorandom comprises two or more stereoisomers.

Subject and related terms: As used herein, the term “subject”, “human subject”, “test subject” and related terms, as used herein, refer to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human being or other mammal. In some embodiments, a subject can be male or female. In non-limiting examples, the animal is a vertebrate such as a primate, rodent, domestic animal or game animal. In non-limiting examples, primates include chimpanzees, cynomologous monkeys, spider monkeys, and macaques, e.g., Rhesus. Rodents include mice, rats, woodchucks, ferrets, rabbits and hamsters. In non-limiting examples, domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate, e.g., a human. In non-limiting examples, the mammal can be a human, non-human primate, mouse, rat, dog, cat, horse, or cow, but are not limited to these examples. In some embodiments, a mammal other than a human can be advantageously used as subjects that represent animal models of disorders associated with autoimmune disease or inflammation. In some embodiments, a method and composition described herein can be used to treat domesticated animals and/or pets.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Targeting compound or moiety or component: The term “targeting moiety”, “targeting compound or moiety”, “targeting compound”, “target component”, and the like, as used herein, is a structure capable of targeting a compound or composition to a particular cell or tissue or subset of cells or tissues. In some embodiments, a targeting moiety is designed to take advantage of cell- or tissue-specific expression of particular targets, receptors, proteins, or other subcellular components; In some embodiments, a targeting moiety is a ligand (e.g., a small molecule, antibody, peptide, protein, carbohydrate, aptamer, etc.) that targets a compound or a composition to a cell or tissue, and/or binds to a target, receptor, protein, or other subcellular component. In some embodiments, a targeting moiety targets a composition comprising a lipid and a biologically active agent to a muscle cell or tissue. In some embodiments, a targeting moiety comprises a compound that targets a muscle cell or tissue. In some embodiments, a targeting moiety comprises fetuin, epidermal growth factor, fibroblast growth factor, insulin, and/or dexamethasone, or a component or fragment or combination thereof. In some embodiments, a targeting moiety targets a composition comprising a lipid and a biologically active agent to a neuron or other cell or tissue in the neuromuscular system. In some embodiments, a targeting moiety comprises a rabies virus peptide (see Kumar et al. 2007 Nature 448: 39-43; and Hwang do et al. 2011 Biomaterials 32: 4968-4975). In some embodiments, a targeting moiety is a moiety capable of binding to a neurotransmitter transporter, a dopamine transporter, a serotonin transporter, or norepinephrine transporter, or alpha-synuclein, or a mRNA encoding any of these components (see U.S. Pat. No. 9,084,825). In some embodiments, a targeting moiety is a transferrin receptor ligand or alpha-transferrin antibody, thus reportedly making use of a transferrin receptor-mediated route across the vascular endothelium. Clark et al. 2015 Proc. Natl. Acad. Sci. USA 112: 12486-12491; Bien-Ly et al. 2014 J. Exp. Med. 211: 233-244; and Youn et al. 2014 Mol. Pharm. 11: 486-495. In some embodiments, a targeting moiety binds to an integrin. In some embodiments, a targeting moiety binds to alphaIIbeta3, e.g., on platelets. In some embodiments, a targeting moiety binds to a beta2 integrin, e.g., on a leukocyte. In some embodiments, a targeting moiety binds to an alphavbeta3, e.g., on a tumor cell. In some embodiments, a targeting moiety binds to a GPCR (G protein-coupled receptor) (see Hanyaloglu et al. 2008 Ann. Rev. Pharm. Tox. 48: 537-568). In some embodiments, a targeting moiety binds to a gastrin releasing peptide receptor, e.g., on a cancer cell (see Cornelio et al. 2007 Ann. Oncol. 18: 1457-1466). In some embodiments, a targeting moiety comprises a carbonic anhydrase inhibitor.

Tautomeric forms: The phrase “tautomeric forms,” as used herein, is used to describe different isomeric forms of organic compounds that are capable of facile interconversion. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present invention. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the invention, chemical compositions may be provided that are or include pure preparations of a single tautomeric form of a compound. In some embodiments of the invention, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the invention, chemical compositions may contain all tautomeric forms of a compound. In some embodiments of the invention, chemical compositions may contain less than all tautomeric forms of a compound. In some embodiments of the invention, chemical compositions may contain one or more tautomeric forms of a compound in amounts that vary over time as a result of interconversion. In some embodiments of the invention, the tautomerism is keto-enol tautomerism. One of skill in the chemical arts would recognize that a keto-enol tautomer can be “trapped” (i.e., chemically modified such that it remains in the “enol” form) using any suitable reagent known in the chemical arts to provide an enol derivative that may subsequently be isolated using one or more suitable techniques known in the art. Unless otherwise indicated, the present invention encompasses all tautomeric forms of relevant compounds, whether in pure form or in admixture with one another.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated” as used herein, means that a moiety has one or more units of unsaturation.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Vaccine: The term “vaccine”, as used herein, refers to a molecule that improves immunity to a particular disease or infectious agent. Vaccines encoded in the polynucleotides, primary constructs or mmRNA of the invention may be utilized to treat conditions or diseases in many therapeutic areas such as, but not limited to, cardiovascular, CNS, dermatology, endocrinology, oncology, immunology, respiratory, and anti-infective. In some embodiments, a vaccine comprises an agent that immunologically resembles a disease-causing micro-organism or fragment thereof; In some embodiments, a vaccine is made from weakened or killed forms of the virus, microbe, parasite or other pathogen, or a fragment thereof. In some embodiments, a vaccine stimulates the body's immune system to recognize the agent as a threat, destroy it, and keep a record of it, so that the immune system can more easily recognize and destroy any of these micro-organisms that it later encounters. In some embodiments, a vaccine is prophylactic or therapeutic. In various embodiments, a vaccine can be to a virus, a bacterium, a parasite, or another pathogen. In some embodiments, a vaccine is to a virus selected from: common cold virus, Hepatitis A virus, Hepatitis B virus, Hepatitis E virus, Human papillomavirus, Influenza virus, Japanese encephalitis virus, Measles virus, Mumps virus, Polio virus, Rabies virus, Rhinovirus, Rotavirus, Rubella virus, Varicella zoster virus, Variola virus, and Yellow fever virus. In various embodiments, a vaccine is a vaccine selected from: a virus vaccine, Adenovirus vaccine, Coxsackie B virus vaccine, Cytomegalovirus vaccine, Dengue vaccine for humans, Eastern Equine encephalitis virus vaccine for humans, Ebola vaccine, Enterovirus 71 vaccine, Epstein-Barr vaccine, Hepatitis C vaccine, HIV vaccine, HTLV-1 T-lymphotropic leukemia vaccine for humans, Marburg virus disease vaccine, Norovirus vaccine, Respiratory syncytial virus vaccine for humans, Severe acute respiratory syndrome (SARS) vaccine, West Nile virus vaccine for humans, and Zika virus vaccine. In some embodiments, a vaccine is to a bacterium selected from: Bacillus anthracis, Vibrio cholerae, Bordetella pertussis, Clostridium tetani, Corynebacterium diphtheriae, Haemophilus influenzae type B (Hib), Neisseria meningitidis, Streptococcus pneumoniae, Coxiella burnetii, Mycobacterium tuberculosis, and Salmonella typhi. In various embodiments, a vaccine is a vaccine selected from: a Bacterial disease vaccine, Caries vaccine, Ehrlichiosis vaccine, Leprosy vaccine, Lyme disease vaccine, Staphylococcus aureus vaccine, Streptococcus pyogenes vaccine, Syphilis vaccine, Tularemia vaccine, and Yersinia pestis vaccine. In various embodiments, a vaccine is a vaccine selected from: A parasitic disease vaccine, Malaria vaccine, Schistosomiasis vaccine, Chagas disease vaccine, Hookworm vaccine, Onchocerciasis river blindness vaccine for humans, Trypanosomiasis vaccine, and Visceral leishmaniasis vaccine. In various embodiments, a vaccine is selected from: a non-infectious disease vaccine, Alzheimer's disease amyloid protein vaccine, Breast cancer vaccine, Ovarian cancer vaccine, Prostate cancer vaccine, and Talimogene laherparepvec (T-VEC). In some embodiments, a composition comprises a lipid and a portion of a vaccine capable of mediating at least one function of a vaccine.

Wild-type: As used herein, the term “wild-type” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms (e.g., alleles).

Nucleic acid: The term “nucleic acid”, as used herein, includes any nucleotides, analogs, and polymers thereof. The term “polynucleotide” as used herein refers to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges or internucleotidic linkage. The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. The prefix poly- refers to a nucleic acid containing 2 to about 10,000 nucleotide monomer units and wherein the prefix oligo- refers to a nucleic acid containing 2 to about 200 nucleotide monomer units. In some embodiments, a nucleic acid includes, but not limited to, deoxyribonucleotides or ribonucleotides and polymers thereof, for example, in at least partially single- or double-stranded form. In some embodiments, a nucleic acid includes any nucleotides, modified nucleotides, and/or nucleotide analogs, and polymers thereof. In some embodiments, a polynucleotide includes a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. Analogs of RNA and DNA (e.g., nucleotide analogs) include, but are not limited to: Morpholino, PNA, LNA, BNA, TNA, GNA, ANA, FANA, CeNa, HNA and UNA. Modified nucleotides include those which are modified in the phosphate, sugar, and/or base. Such modifications include sugar modifications at the 2′ carbon, such as 2′-MOE, 2′-OMe, and 2′-F. In some embodiments, a nucleic acid includes a poly- or oligo-ribonucleotide (RNA) and poly- or oligo-deoxyribonucleotide (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges or internucleotidic linkage. The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, a nucleic acid is an oligonucleotide, an antisense oligonucleotide, an RNAi agent, a miRNA, splice switching oligonucleotide (SSO), immunomodulatory nucleic acid, an aptamer, a ribozyme, a Piwi-interacting RNA (piRNA), a small nucleolar RNA (snoRNA), a mRNA, a lncRNA, a ncRNA, an antigomir (e.g., an antagonist to a miRNA, lncRNA, ncRNA or other nucleic acid), a plasmid, a vector, or a portion thereof. In some embodiments, a nucleic acid is a chirally controlled nucleic acid composition. In some embodiments, the biologically active agent is a chirally controlled oligonucleotide composition, or a chirally controlled nucleic acid composition. In some embodiments, a base, nucleobase, nitrogenous base, heterocyclic base and the like includes a part (or a modified variant thereof) of a nucleic acid that is involved in the hydrogen-bonding that binds one nucleic acid strand to another complementary strand in a sequence-specific manner. The naturally occurring bases, [guanine, (G), adenine, (A), cytosine, (C), thymine, (T), and uracil (U)] are derivatives of purine (Pu) or pyrimidine (Py), though it should be understood that naturally and non-naturally occurring base analogs are also included. In some embodiments, the nucleobases are modified adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. Various additional modifications of the base are known in the art. In some cases, a nucleic acid sequence may be defined as a sequence of bases, generally presented in the 5′ to 3′ direction. While in the context of a nucleic acid, a base is normally conjugated to a sugar which forms the backbone along with an internucleotidic linkage (e.g., a phosphate or phosphorothioate); however, as used herein, the term “base” does not comprise a sugar or an internucleotidic linkage. In some embodiments, a nucleoside includes a unit consisting of: (a) a base covalently bound to (b) a sugar. The base and/or sugar can be modified or not modified. In some embodiments, a sugar, as referenced herein in the context of referencing a nucleic acid, includes to a monosaccharide in closed and/or open form. The naturally occurring sugar is the pentose (five-carbon sugar) deoxyribose (which forms DNA) or ribose (which forms RNA), though it should be understood that naturally and non-naturally occurring sugar analogs are also included. Sugars include, but are not limited to, ribose, deoxyribose, pentofuranose, pentopyranose, and hexopyranose moieties. As used herein, the term also encompasses structural analogs used in lieu of conventional sugar molecules, such as glycol, polymer of which forms the backbone of the nucleic acid analog, glycol nucleic acid (“GNA”). A deoxynucleoside comprises a deoxyribose. In some cases, a nucleic acid sequence may be defined as a sequence of bases and sugar modifications. In some embodiments, a sugar includes a modified sugar or unmodified sugar. In some embodiments, a modified sugar includes, as referenced in the context of a nucleic acid, a sugar which has been modified or a moiety that can functionally replace a sugar in a nucleic acid or modified nucleic acid. The modified sugar mimics the spatial arrangement, electronic properties, or some other physicochemical property of a sugar. A modified sugar, as a non-limiting example, can have a modification at the 2′ carbon. Various modifications include 2′-MOE, 2′-OMe and 2′-F. Various additional modifications of the sugar are known in the art. In some embodiments, a nucleotide includes to a monomeric unit of a polynucleotide that consists of: (a) a heterocyclic base, a sugar, and one or more phosphate groups or phosphorus-containing internucleotidic linkages; a nucleotide is a subunit of a polynucleotide, nucleic acid or oligonucleotide. Each base, sugar and phosphate or internucleoside linker can be independently modified or not modified. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, an internucleotidic linkage includes linkage between nucleoside units of an oligonucleotide; in most cases the linkage comprises a phosphorus or linkage phosphorus; in some embodiments, the linkage is referred to as “p”. In some embodiments, an internucleotidic linkage is a phosphodiester linkage, as found in naturally occurring DNA and RNA molecules. In some embodiments, the linkage is a phosphorothioate. In some embodiments, the backbone of an oligonucleotide or a nucleic acid includes the alternating sugars and internucleotidic linkages (e.g., a phosphodiester or phosphorothioate). Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Also included are molecules having naturally occurring phosphodiester linkages as well as those having non-naturally occurring linkages, e.g., for stabilization purposes. The nucleic acid may be in any physical form, e.g., linear, circular, or supercoiled. The term nucleic acid is used interchangeably with oligonucleotide, gene, cDNA, and mRNA encoded by a gene. In various embodiments, one or more nucleotides is modified or is substituted with one or more DNA, a peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), constrained ethyl (cEt), tricyclo-DNA (tc-DNA), xeno nucleic acid (XNA), and/or unlocked nucleic acid (UNA). In various embodiments, the nucleic acid comprises a modified internucleoside linker.

Nucleotide: The term “nucleotide” as used herein refers to a monomeric unit of a polynucleotide that consists of a heterocyclic base, a sugar, and one or more phosphate groups or phosphorus-containing internucleotidic linkages. The naturally occurring bases, (guanine, (G), adenine, (A), cytosine, (C), thymine, (T), and uracil (U)) are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. As described herein, in some embodiments, a nucleotide is a natural nucleotide; in some embodiments, a nucleotide is modified.

Nucleoside: The term “nucleoside”, as used herein, refers to a moiety wherein a nucleobase or a modified nucleobase is covalently bound to a sugar or modified sugar.

Sugar: The term “sugar”, as used herein, refers to a saccharide, in some embodiments, a monosaccharide in closed and/or open form. Sugars include, but are not limited to, ribose, deoxyribose, pentofuranose, pentopyranose, and hexopyranose moieties. As used herein, the term also encompasses structural analogs used in lieu of conventional sugar molecules, such as glycol, polymer of which forms the backbone of the nucleic acid analog, glycol nucleic acid (“GNA”).

Modified sugar: The term “modified sugar”, as used herein, refers to a moiety that can replace a sugar, in some embodiments, in oligonucleotides. The modified sugar mimics the spatial arrangement, electronic properties, or some other physicochemical property of a sugar.

Nucleobase: The term “nucleobase”, as used herein, refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, the naturally-occurring nucleobases are modified adenine, guanine, uracil, cytosine, or thymine. In some embodiments, the naturally-occurring nucleobases are methylated adenine, guanine, uracil, cytosine, or thymine. In some embodiments, a nucleobase is a “modified nucleobase,” e.g., a nucleobase other than adenine (A), guanine (G), uracil (U), cytosine (C), and thymine (T). In some embodiments, the modified nucleobases are methylated adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex.

Chiral ligand: The term “chiral ligand” or “chiral auxiliary”, as used herein, refers to a moiety that is chiral and can be incorporated into a reaction so that the reaction can be carried out with certain stereoselectivity.

Condensing reagent: In a condensation reaction, the term “condensing reagent”, as used herein, refers to a reagent that activates a less reactive site and renders it more susceptible to attack by another reagent. In some embodiments, such another reagent is a nucleophile.

Blocking group: The term “blocking group”, as used herein, refers to a group that masks the reactivity of a functional group. The functional group can be subsequently unmasked by removal of the blocking group. In some embodiments, a blocking group is a protecting group.

Moiety: The term “moiety”, as used herein, refers to a specific segment or functional group of a molecule. Chemical moieties are often recognized chemical entities embedded in or appended to a molecule.

Solid support: The term “solid support”, as used herein, refers to any support which enables synthesis of nucleic acids. In some embodiments, the term refers to a glass or a polymer, that is insoluble in the media employed in the reaction steps performed to synthesize nucleic acids, and is derivatized to comprise reactive groups. In some embodiments, the solid support is Highly Cross-linked Polystyrene (HCP) or Controlled Pore Glass (CPG). In some embodiments, the solid support is Controlled Pore Glass (CPG). In some embodiments, the solid support is hybrid support of Controlled Pore Glass (CPG) and Highly Cross-linked Polystyrene (HCP).

Coding sequence: A DNA “coding sequence” or “coding region” is a double-stranded DNA sequence which is transcribed and translated into a polypeptide in vivo when placed under the control of appropriate expression control sequences. The boundaries of the coding sequence (the “open reading frame” or “ORF”) are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence is, usually, be located 3′ to the coding sequence. The term “non-coding sequence” or “non-coding region” refers to regions of a polynucleotide sequence that are not translated into amino acids (e.g. 5′ and 3′ un-translated regions).

Reading frame: The term “reading frame”, as used herein, refers to one of the six possible reading frames, three in each direction, of the double stranded DNA molecule. The reading frame that is used determines which codons are used to encode amino acids within the coding sequence of a DNA molecule.

Homology: The terms “Homology” or “identity” or “similarity”, as used herein, refers to sequence similarity between two nucleic acid molecules. Homology and identity can each be determined by comparing a position in each sequence which can be aligned for purposes of comparison. When an equivalent position in the compared sequences is occupied by the same base, then the molecules are identical at that position; when the equivalent site occupied by the same or a similar nucleic acid residue (e.g., similar in steric and/or electronic nature), then the molecules can be referred to as homologous (similar) at that position. Expression as a percentage of homology/similarity or identity refers to a function of the number of identical or similar nucleic acids at positions shared by the compared sequences. A sequence which is “unrelated” or “non-homologous” shares less than 40% identity, less than 35% identity, less than 30% identity, or less than 25% identity with a sequence described herein. In comparing two sequences, the absence of residues (amino acids or nucleic acids) or presence of extra residues also decreases the identity and homology/similarity. In some embodiments, the term “homology” describes a mathematically based comparison of sequence similarities which is used to identify genes with similar functions or motifs. The nucleic acid sequences described herein can be used as a “query sequence” to perform a search against public databases, for example, to identify other family members, related sequences or homologs. In some embodiments, such searches can be performed using the NBLAST and XBLAST programs (version 2.0) of Altschul, et al. (1990) J. Mol. Biol. 215:403-10. In some embodiments, BLAST nucleotide searches can be performed with the NBLAST program, score=100, word length=12 to obtain nucleotide sequences homologous to nucleic acid molecules of the invention. In some embodiments, to obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (See www.ncbi.nlm.nih.gov).

Identity: As used herein, “identity” means the percentage of identical nucleotide residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm can also be used to determine identity.

Heterologous: A “heterologous” region of a DNA sequence is an identifiable segment of DNA within a larger DNA sequence that is not found in association with the larger sequence in nature. Thus, when the heterologous region encodes a mammalian gene, the gene can usually be flanked by DNA that does not flank the mammalian genomic DNA in the genome of the source organism. Another example of a heterologous coding sequence is a sequence where the coding sequence itself is not found in nature (e.g., a cDNA where the genomic coding sequence contains introns or synthetic sequences having codons or motifs different than the unmodified gene). Allelic variations or naturally-occurring mutational events do not give rise to a heterologous region of DNA as defined herein.

Oligonucleotide: The term “oligonucleotide”, as used herein, refers to a polymer or oligomer of nucleotide monomers, containing any combination of nucleobases, modified nucleobases, sugars, modified sugars, phosphate bridges, or modified phosphorus atom bridges (also referred to herein as “internucleotidic linkage”, defined further herein).

Oligonucleotides can be single-stranded or double-stranded. As used herein, the term “oligonucleotide strand” encompasses a single-stranded oligonucleotide. A single-stranded oligonucleotide can have double-stranded regions and a double-stranded oligonucleotide can have single-stranded regions. Example oligonucleotides include, but are not limited to structural genes, genes including control and termination regions, self-replicating systems such as viral or plasmid DNA, single-stranded and double-stranded siRNAs and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Double-stranded and single-stranded oligonucleotides that are effective in inducing RNA interference are also referred to as siRNA, RNAi agent, or iRNA agent, herein. In some embodiments, these RNA interference inducing oligonucleotides associate with a cytoplasmic multi-protein complex known as RNAi-induced silencing complex (RISC). In many embodiments, single-stranded and double-stranded RNAi agents are sufficiently long that they can be cleaved by an endogenous molecule, e.g., by Dicer, to produce smaller oligonucleotides that can enter the RISC machinery and participate in RISC mediated cleavage of a target sequence, e.g. a target mRNA.

Oligonucleotides of the present invention can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length. In some embodiments, a sequence of a nucleic acid or an oligonucleotide comprises or consists of a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, a sequence of a nucleic acid or an oligonucleotide comprises or consists of a common base sequence hybridizes with a transcript of a gene related to Huntington's disease, spinal muscular atrophy, spinal muscular atrophy type 1, amyotrophic lateral sclerosis, Duchenne muscular dystrophy, myotonic dystrophy, myotonic dystrophy type 1, a genetic disease of the liver, a metabolic disease of the liver, epidermolysis bullosa simplex, a genetic disease of the skin, a genetic disease of the skin, or irritable bowel syndrome, or a genetic disease, or a metabolic disease.

Internucleotidic linkage: As used herein, the phrase “internucleotidic linkage” refers generally to the phosphorus-containing linkage between nucleotide units of an oligonucleotide, and is interchangeable with “inter-sugar linkage” and “phosphorus atom bridge,” as used above and herein. In some embodiments, an internucleotidic linkage is a phosphodiester linkage, as found in naturally occurring DNA and RNA molecules. In some embodiments, an internucleotidic linkage is a modified phosphodiester linkage. In some embodiments, an internucleotidic linkage is a “modified internucleotidic linkage” wherein each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described below. In some embodiments, an internucleotidic linkage is a phosphotriester linkage, phosphorothioate diester linkage

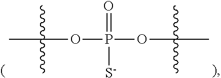

or modified phosphorothioate triester linkage. It is understood by a person of ordinary skill in the art that the internucleotidic linkage may exist as an anion or cation at a given pH due to the existence of acid or base moieties in the linkage.

Unless otherwise specified, when used with an oligonucleotide sequence, each of s, s1, s2, s3, s4, s5, s6 and s7 independently represents the following modified internucleotidic linkage as illustrated in Table 2, below.

TABLE 2
Example Modified Internucleotidic Linkage.

| Symbol | Modified Internucleotidic Linkage |
|---|---|
| s | 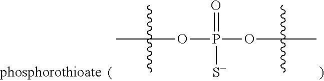 [SMILES: *OP(=O)([S-])O*] |
| s1 | 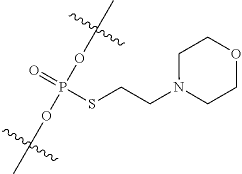 [SMILES: *OP(=O)(O*)SCCN1CCOCC1] |
| s2 | 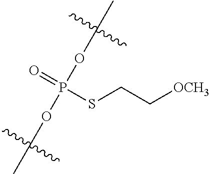 [SMILES: *OP(=O)(O*)SCCOC] |
| s3 | 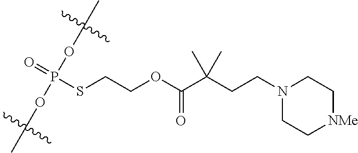 [SMILES: *OP(=O)(O*)SCCOC(=O)C(C)(C)CCN1CCN(C)CC1] |
| s4 | 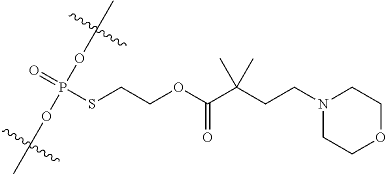 [SMILES: *OP(=O)(O*)SCCOC(=O)C(C)(C)CCN1CCOCC1] |
| s5 | 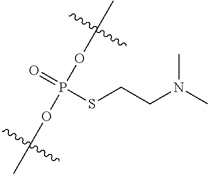 [SMILES: *OP(=O)(O*)SCCN(C)C] |
| s6 | 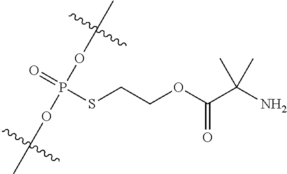 [SMILES: *OP(=O)(O*)SCCOC(=O)C(C)(C)N] |
| s7 | 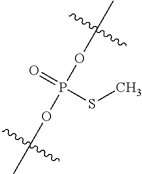 [SMILES: *OP(=O)(O*)SC] |
| s8 | 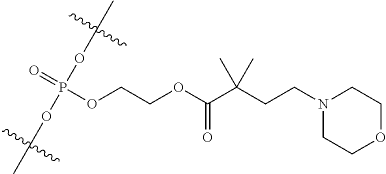 [SMILES: *OP(=O)(O*)OCCOC(=O)C(C)(C)CCN1CCOCC1] |
| s9 | 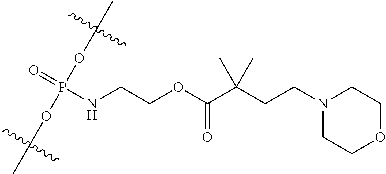 [SMILES: *OP(=O)(NCCOC(=O)C(C)(C)CCN1CCOCC1)O*] |
| s10 | 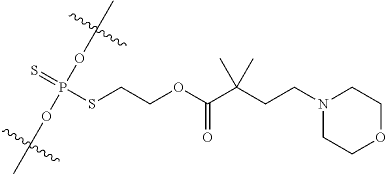 [SMILES: *OP(=S)(O*)SCCOC(=O)C(C)(C)CCN1CCOCC1] |
| s11 | 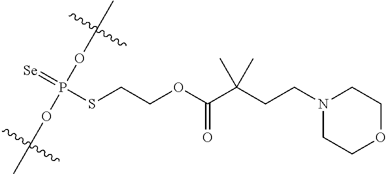 [SMILES: *OP(=[Se])(O*)SCCOC(=O)C(C)(C)CCN1CCOCC1] |
| s12 | 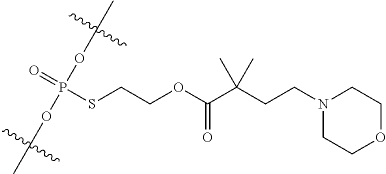 [SMILES: *OP(=O)(O*)SCCOC(=O)C(C)(C)CCN1CCOCC1] |
| s13 | 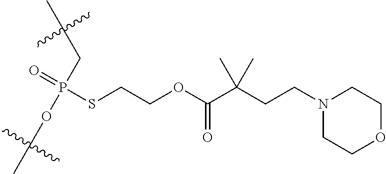 [SMILES: *CP(=O)(O*)SCCOC(=O)C(C)(C)CCN1CCOCC1] |
| s14 | 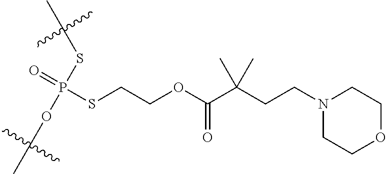 [SMILES: *OP(=O)(S*)SCCOC(=O)C(C)(C)CCN1CCOCC1] |
| s15 | 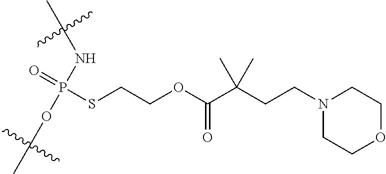 [SMILES: *NP(=O)(O*)SCCOC(=O)C(C)(C)CCN1CCOCC1] |
| s16 | 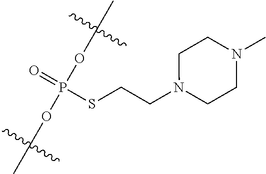 [SMILES: *OP(=O)(O*)SCCN1CCN(C)CC1] |
| s17 | 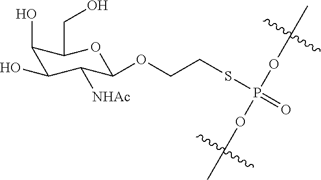 [SMILES: *OP(=O)(O*)SCCOC1OC(CO)C(O)C(O)C1NC(C)=O] |
| s18 | 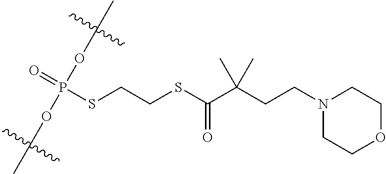 [SMILES: *OP(=O)(O*)SCCSC(=O)C(C)(C)CCN1CCOCC1] |

For instance, (Rp, Sp)-ATsCs1GA has 1) a phosphorothioate internucleotidic linkage

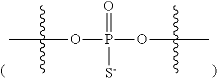

between T and C; and 2) a phosphorothioate triester internucleotidic linkage having the structure of

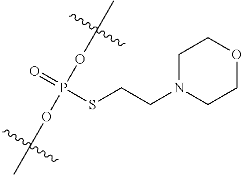 [SMILES: *OP(=O)(O*)SCCN1CCOCC1]

between C and G. Unless otherwise specified, the Rp/Sp designations preceding an oligonucleotide sequence describe the configurations of chiral linkage phosphorus atoms in the internucleotidic linkages sequentially from 5′ to 3′ of the oligonucleotide sequence. For instance, in (Rp, Sp)-ATsCs1GA, the phosphorus in the “s” linkage between T and C has Rp configuration and the phosphorus in “s1” linkage between C and G has Sp configuration. In some embodiments, “All-(Rp)” or “All-(Sp)” is used to indicate that all chiral linkage phosphorus atoms in oligonucleotide have the same Rp or Sp configuration, respectively. For instance, All-(Rp)-GsCsCsTsCsAsGsTsCsTsGsCsTsTsCsGsCsAsCsC (SEQ ID NO: 3) indicates that all the chiral linkage phosphorus atoms in the oligonucleotide have Rp configuration; All-(Sp)-GsCsCsTsCsAsGsTsCsTsGsCsTsTsCsGsCsAsCsC (SEQ ID NO: 4) indicates that all the chiral linkage phosphorus atoms in the oligonucleotide have Sp configuration.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, etc), pattern of backbone chiral centers (i.e. pattern of linkage phosphorus stereochemistry (Rp/Sp)), and pattern of backbone phosphorus modifications (e.g., pattern of “—XLR¹” groups in formula I). In some embodiments, oligonucleotides of a common designated “type” are structurally identical to one another.

One of skill in the art will appreciate that synthetic methods of the present invention provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. The present invention provides compositions comprising or consisting of a plurality of oligonucleotide molecules (e.g., chirally controlled oligonucleotide compositions). In some embodiments, all such molecules are of the same type. In some embodiments, provided compositions comprise a plurality of oligonucleotides of different types, typically in pre-determined relative amounts.

Chiral control: As used herein, “chiral control” refers to an ability to control the stereochemical designation of a chiral linkage phosphorus in a chiral internucleotidic linkage within an oligonucleotide. In some embodiments, a control is achieved through a chiral element that is absent from the sugar and base moieties of an oligonucleotide, for example, in some embodiments, a control is achieved through use of one or more chiral auxiliaries during oligonucleotide preparation as exemplified in the present disclosure. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage.

Chirally controlled oligonucleotide composition: The terms “chirally controlled oligonucleotide composition”, “chirally controlled nucleic acid composition”, and the like, as used herein, refers to a composition that comprising a plurality of oligonucleotides (or nucleic acids) which share 1) a common base sequence, 2) a common pattern of backbone linkages, and 3) a common pattern of backbone phosphorus modifications, wherein the plurality of oligonucleotides share the same stereochemistry at one or more chiral internucleotidic linkages (chirally controlled internucleotidic linkages), and the level of the plurality of oligonucleotides in the composition is pre-determined. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type. In some embodiments, a chirally controlled oligonucleotide composition comprises more than one oligonucleotide type. In some embodiments, a chirally controlled oligonucleotide composition comprises multiple oligonucleotide types.

Chirally pure: As used herein, the phrase “chirally pure” is used to describe a chirally controlled oligonucleotide composition, or a plurality of oligonucleotides, in which all of the oligonucleotides exist in a single diastereomeric form with respect to the linkage phosphorus.

Chirally uriform: As used herein, the phrase “chirally uniform” is used to describe an oligonucleotide molecule or type in which all nucleotide units have the same stereochemistry at the linkage phosphorus. For instance, an oligonucleotide whose nucleotide units all have Rp stereochemistry at the linkage phosphorus is chirally uniform. Likewise, an oligonucleotide whose nucleotide units all have Sp stereochemistry at the linkage phosphorus is chirally uniform.

Predetermined: By predetermined (or pre-determined) is meant deliberately selected, for example as opposed to randomly occurring or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that cannot be controlled to intentionally generate the particular chemistry and/or stereochemistry features is not a “predetermined” composition. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled.

Linkage phosphorus: As defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester of an internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is P* of formula I. In some embodiments, a linkage phosphorus atom is chiral. In some embodiments, a chiral linkage phosphorus atom is P* of formula I.

P-modification: As used herein, the term “P-modification” refers to any modification at the linkage phosphorus other than a stereochemical modification. In some embodiments, a P-modification comprises addition, substitution, or removal of a pendant moiety covalently attached to a linkage phosphorus. In some embodiments, the “P-modification” is —X-L-R¹ wherein each of X, L and R¹ is independently as defined and described herein and below.

Blockmer: The term “blockmer,” as used herein, refers to an oligonucleotide strand whose pattern of structural features characterizing each individual nucleotide unit is characterized by the presence of at least two consecutive nucleotide units sharing a common structural feature at the internucleotidic phosphorus linkage. By common structural feature is meant common stereochemistry at the linkage phosphorus or a common modification at the linkage phosphorus. In some embodiments, the at least two consecutive nucleotide units sharing a common structure feature at the internucleotidic phosphours linkage are referred to as a “block”.

In some embodiments, a blockmer is a “stereoblockmer,” e.g., at least two consecutive nucleotide units have the same stereochemistry at the linkage phosphorus. Such at least two consecutive nucleotide units form a “stereoblock.” For instance, (Sp, Sp)-ATsCs1GA is a stereoblockmer because at least two consecutive nucleotide units, the Ts and the Cs1, have the same stereochemistry at the linkage phosphorus (both Sp). In the same oligonucleotide (Sp, Sp)-ATsCs1GA, TsCs1 forms a block, and it is a stereoblock.

In some embodiments, a blockmer is a “P-modification blockmer,” e.g., at least two consecutive nucleotide units have the same modification at the linkage phosphorus. Such at least two consecutive nucleotide units form a “P-modification block”. For instance, (Rp, Sp)-ATsCsGA is a P-modification blockmer because at least two consecutive nucleotide units, the Ts and the Cs, have the same P-modification (i.e., both are a phosphorothioate diester). In the same oligonucleotide of (Rp, Sp)-ATsCsGA, TsCs forms a block, and it is a P-modification block.

In some embodiments, a blockmer is a “linkage blockmer,” e.g., at least two consecutive nucleotide units have identical stereochemistry and identical modifications at the linkage phosphorus. At least two consecutive nucleotide units form a “linkage block”. For instance, (Rp, Rp)-ATsCsGA is a linkage blockmer because at least two consecutive nucleotide units, the Ts and the Cs, have the same stereochemistry (both Rp) and P-modification (both phosphorothioate). In the same oligonucleotide of (Rp, Rp)-ATsCsGA, TsCs forms a block, and it is a linkage block.

In some embodiments, a blockmer comprises one or more blocks independently selected from a stereoblock, a P-modification block and a linkage block. In some embodiments, a blockmer is a stereoblockmer with respect to one block, and/or a P-modification blockmer with respect to another block, and/or a linkage blockmer with respect to yet another block. For instance, (Rp, Rp, Rp, Rp, Rp, Sp, Sp, Sp)-AAsTsCsGsAs1Ts1Cs1Gs1ATCG (SEQ ID NO: 5) is a stereoblockmer with respect to the stereoblock AsTsCsGsAs1 (all Rp at linkage phosphorus) or Ts1Cs1Gs1 (all Sp at linkage phosphorus), a P-modification blockmer with respect to the P-modification block AsTsCsGs (all s linkage) or As1Ts1Cs1Gs1 (all s1 linkage), or a linkage blockmer with respect to the linkage block AsTsCsGs (all Rp at linkage phosphorus and all s linkage) or Ts1Cs1Gs1 (all Sp at linkage phosphorus and all s1 linkage).

Altmer: The term “altmer,” as used herein, refers to an oligonucleotide strand whose pattern of structural features characterizing each individual nucleotide unit is characterized in that no two consecutive nucleotide units of the oligonucleotide strand share a particular structural feature at the internucleotidic phosphorus linkage. In some embodiments, an altmer is designed such that it comprises a repeating pattern. In some embodiments, an altmer is designed such that it does not comprise a repeating pattern.

In some embodiments, an altmer is a “stereoaltmer,” e.g., no two consecutive nucleotide units have the same stereochemistry at the linkage phosphorus. For instance, (Rp, Sp, Rp, Sp, Rp, Sp, Rp, Sp, Rp, Sp Rp, Sp, Rp, Sp, Rp, Sp, Rp, Sp, Rp)-GsCsCsTsCsAsGsTsCsTsGsCsTsTsCsGsCsAsCsC (SEQ ID NO: 6).

In some embodiments, an altmer is a “P-modification altmer” e.g., no two consecutive nucleotide units have the same modification at the linkage phosphorus. For instance, All-(Sp)-CAs1GsT, in which each linkage phosphorus has a different P-modification than the others.

In some embodiments, an altmer is a “linkage altmer,” e.g., no two consecutive nucleotide units have identical stereochemistry or identical modifications at the linkage phosphorus. For instance, (Rp, Sp, Rp, Sp, Rp, Sp, Rp, Sp, Rp, Sp Rp, Sp, Rp, Sp, Rp, Sp, Rp, Sp, Rp)-GsCs1CsTs1CsAs1GsTs1CsTs1GsCs1TsTs2CsGs3CsAs4CsC (SEQ ID NO: 7).

Sequence: As used herein, the term “sequence” refers to any arrangement of molecules or atoms characteristic of a particular molecule. In some embodiments, in referencing a nucleic acid, a “sequence” refers to any of: base sequence (including length), the pattern of chemical modifications to sugar and base moieties, the pattern of backbone linkages (e.g., pattern of natural phosphate linkages, phosphorothioate linkages, phosphorothioate triester linkages, and combinations thereof), the pattern of backbone chiral centers (e.g., pattern of stereochemistry (Rp/Sp) of chiral internucleotidic linkages), and the pattern of backbone phosphorus modifications (e.g., pattern of modifications on the internucleotidic phosphorus atom, such as —S—, and -L-R¹ of formula I). In some embodiments, in referencing a nucleic acid or oligonucleotide, a “sequence” refers to the sequence of bases or base sequence. In some embodiments, in reference to a peptide or protein, a sequence refers to a sequence of amino acids.

Unimer: The term “unimer,” as used herein, refers to an oligonucleotide strand whose pattern of structural features characterizing each individual nucleotide unit is such that all nucleotide units within the strand share at least one common structural feature at the internucleotidic phosphorus linkage. By common structural feature is meant common stereochemistry at the linkage phosphorus or a common modification at the linkage phosphorus.

In some embodiments, a unimer is a “stereounimer,” e.g., all nucleotide units have the same stereochemistry at the linkage phosphorus. For instance, All-(Sp)-CsAs1GsT, in which all the linkages have Sp phosphorus.

In some embodiments, a unimer is a “P-modification unimer”, e.g., all nucleotide units have the same modification at the linkage phosphorus. For instance, (Rp, Sp, Rp, Sp, Rp, Sp, Rp, Sp, Rp, Sp Rp, Sp, Rp, Sp, Rp, Sp, Rp, Sp, Rp)-GsCsCsTsCsAsGsTsCsTsGsCsTsTsCsGsCsAsCsC (SEQ ID NO: 6), in which all the internucleotidic linkages are phosphorothioate diester.

In some embodiments, a unimer is a “linkage unimer,” e.g., all nucleotide units have the same stereochemistry and the same modifications at the linkage phosphorus. For instance, All-(Sp)-GsCsCsTsCsAsGsTsCsTsGsCsTsTsCsGsCsAsCsC (SEQ ID NO: 4), in which all the internucleotidic linkages are phosphorothioate diester having Sp linkage phosphorus.

Gapmer: As used herein, the term “gapmer” refers to an oligonucleotide strand characterized in that at least one internucleotidic phosphorus linkage of the oligonucleotide strand is a phosphate diester linkage, for example such as those found in naturally occurring DNA or RNA. In some embodiments, more than one internucleotidic phosphorus linkage of the oligonucleotide strand is a phosphate diester linkage such as those found in naturally occurring DNA or RNA. For instance, All-(Sp)-CAs1GsT, in which the internucleotidic linkage between C and A is a phosphate diester linkage.

Skipmer: As used herein, the term “skipmer” refers to a type of gapmer in which every other internucleotidic phosphorus linkage of the oligonucleotide strand is a phosphate diester linkage, for example such as those found in naturally occurring DNA or RNA, and every other internucleotidic phosphorus linkage of the oligonucleotide strand is a modified internucleotidic linkage. For instance, All-(Sp)-AsTCs1GAs2TCs3G.

Unless specified otherwise, methods and structures described herein relating to compounds and compositions also apply to pharmaceutically acceptable acid or base addition salts and stereoisomeric forms of these compounds and compositions.

### 2. Detailed Description of Certain Embodiments

Many technologies for delivering biologically active agents can suffer from an inability to target desired cells or tissues. For example, delivery of biologically active agents to tissues outside the liver remains particularly difficult. Juliano reported that, despite advances at the clinical level, effective delivery of oligonucleotides in vivo remains a major challenge, especially at extra-hepatic sites. Juliano 2016 Nucl. Acids Res. Doi: 10.1093/nar/gkw236. Lou also reported that delivery of siRNA to organs beyond the liver remains the biggest hurdle to using the technology for a host of diseases. Lou 2014 SciBX 7(48); doi:10.1038/scibx.2014.1394. In some embodiments, the present disclosure encompasses surprising findings, including that lipids can be particularly effective at delivering biologically active agents to particular cells and tissues, including cells and tissues outside the liver, including, as non-limiting examples, muscle cells and tissues.

Among other things, the present disclosure encompasses the recognition that lipids can surprisingly enable and/or promote delivery of biologically active agents to their target location(s) (e.g., cells, tissues, organs, etc.). In some embodiments, the present disclosure provides compositions comprising a biologically active agent and a lipid. In some embodiments, provided compositions and methods are particularly effective for delivering the biologically active agent therein to target locations. In some embodiments, a target location is a cell. In some embodiments, a target location is a type of cell in a tissue. In some embodiments, a target location is a tissue. In some embodiments, a target location is an organ. In some embodiments, at a target location, a biologically active agent of a provided composition is delivered into a cell, e.g., the cytoplasm, nucleus, etc.

In some embodiments, provided technologies can be utilized to effectively improve delivery of biologically active agents to their target location(s) in a subject, e.g., in a mammal or human subject, etc. In some embodiments, provided technologies provide surprising achievement of efficient and/or effective delivery of biologically active agent(s) into cells (i.e., to intracellular location(s) such as cytoplasm, nucleus, etc.) of a subject.

In some embodiments, provided technologies permit or facilitate delivery of an effective and/or desired amount of biologically active agent to its target location(s) so that, for example, a comparable or higher level of the biologically active agent is achieved at the target location(s) than is observed when the biologically active agent is administered absent the lipid, in some embodiments, even though a lower amount of the biologically active agent may be administered with the lipid than without. In some embodiments, provided technologies permit or facilitate improved distribution (i.e., increased relative level of biologically active agent at a target location(s) as compared with at a non-target location(s)) relative to an appropriate control (e.g., that level observed when the oligonucleotide is comparably administered absent the lipid). In some embodiments, provided technologies render biologically active agents that have otherwise been considered unsuitable for therapeutic use to be successfully used for treating various diseases, disorders and/or conditions.

In some embodiments, provided technologies are particularly effective at delivering biologically active agents to particular types of cells and tissues, including, but not limited to, cells and tissues outside the liver (e.g., extra-hepatic), including, but not limited to, muscle cells and tissues. In some embodiments, the present disclosure provides technologies that are surprisingly effective at delivering biologically active agents to muscle cells and tissues, e.g., of skeletal muscles, gastrocnemius, heart, quadriceps, triceps, and/or thoracic diaphragm, etc. In some embodiments, provided technologies effectively deliver a biologically active agent into cells of gastrocnemius muscle of a subject. In some embodiments, provided technologies effectively deliver a biologically active agent into cells of cardiac muscle of a subject. In some embodiments, provided technologies effectively deliver a biologically active agent into cells of quadriceps of a subject. In some embodiments, provided technologies effectively deliver a biologically active agent into cells of triceps of a subject. In some embodiments, provided technologies effectively deliver a biologically active agent into cells of thoracic diaphragm of a subject.

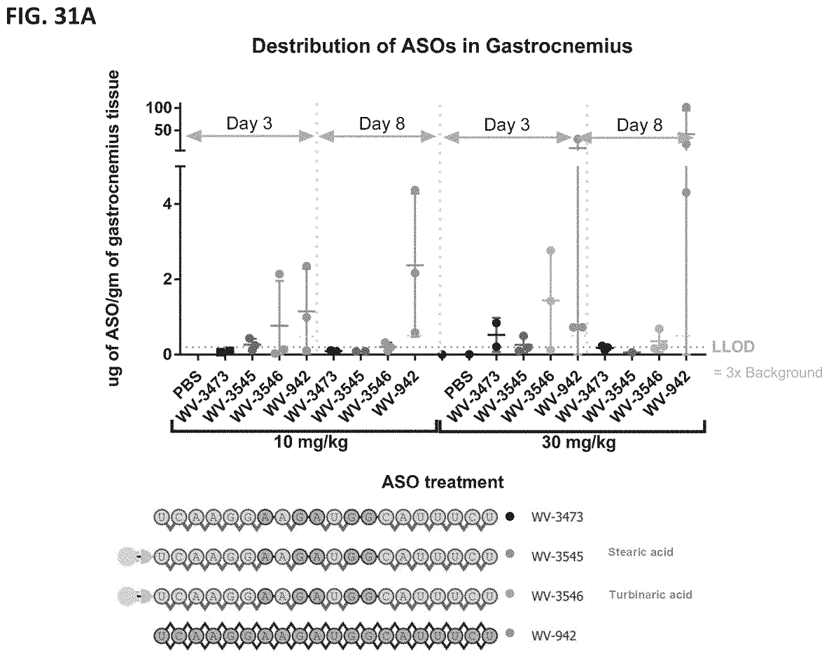

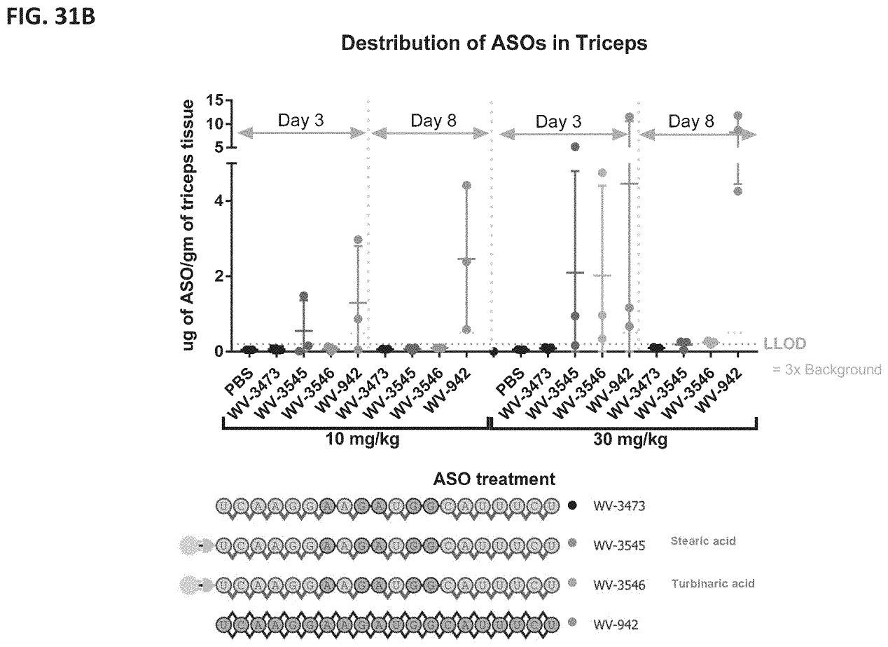

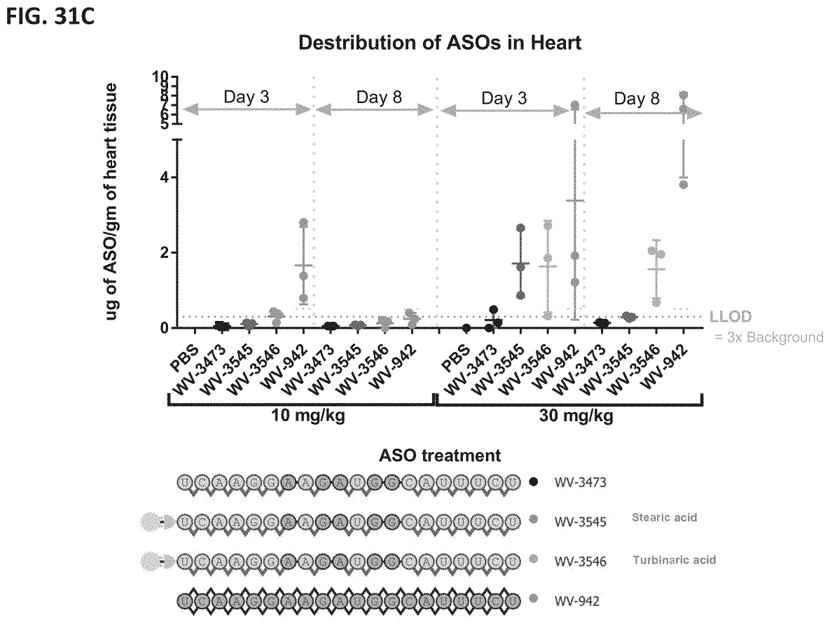

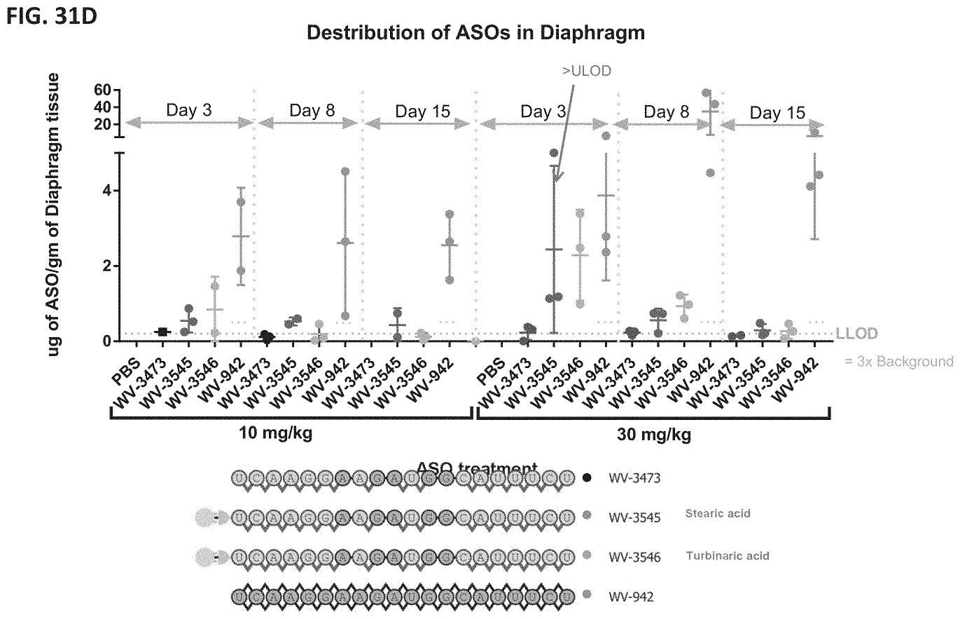

In some embodiments, provided oligonucleotides comprising lipid moieties provide improved delivery to muscles, e.g., gastrocnemius, triceps, heart, diaphragm, etc., compared to reference oligonucleotides, e.g., having no lipid moieties, having no lipid moieties and different stereochemistry (e.g., chirally controlled v. stereorandom, one pattern of backbone chiral centers v. another pattern of backbone chiral centers, etc.), etc. In some embodiments, provided oligonucleotides comprising lipid moieties provide improved pharmacokinetics compared to reference oligonucleotides. In some embodiments, provided oligonucleotides provides faster clearance from a system than reference oligonucleotides, which, as appreciated by a person having ordinary skill in the art, may provide lower toxicities compared to reference oligonucleotides. Example data are presented in FIGS. 31A to 31D.

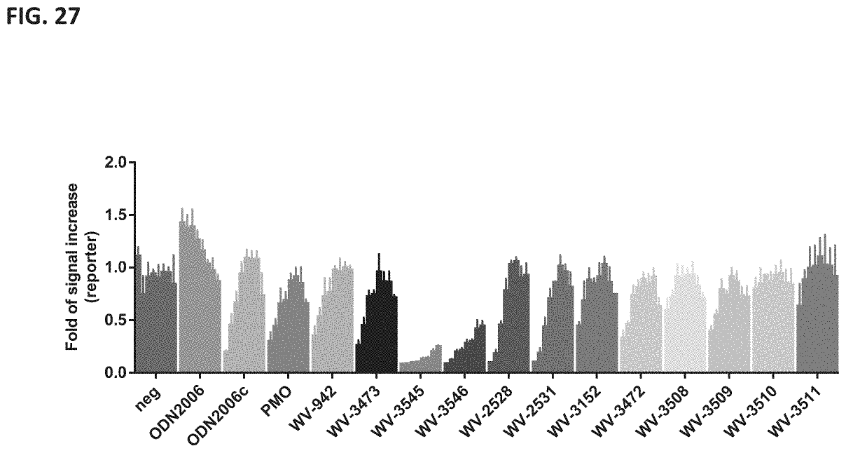

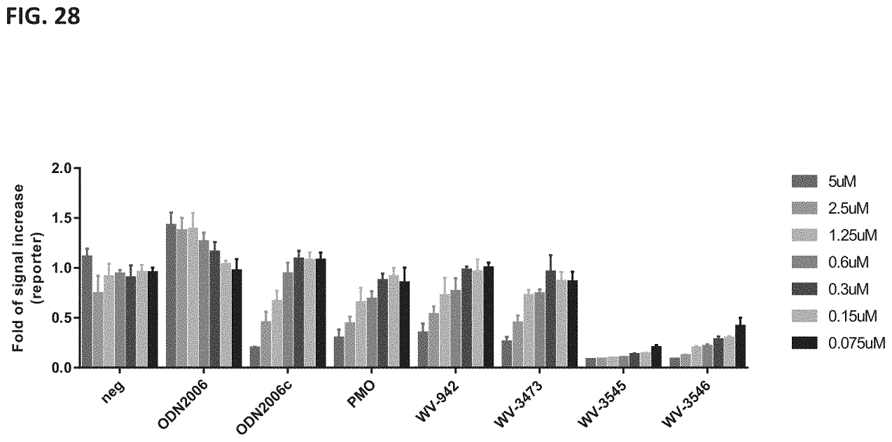

In some embodiments, provided technologies are particularly effective at improving immunogenic properties of biologically active agents. In some embodiments, conjugation of a biologically active agent with a lipid can reduce the immunogenicity of the biologically active agent. In some embodiments, conjugation of a biologically active agent with a lipid can enhance the ability of the biologically active agent to antagonize an immune response. In some embodiments, conjugation of a biologically active agent with a lipid can enhance the ability of the biologically active agent to antagonize an immune response, wherein the immune response is mediated at least partially by TLR9. In some embodiments, conjugation of a lipid to an oligonucleotide improves at least one property of the oligonucleotide. In some embodiments, improved properties include increased activity (e.g., increased ability to induce desirable skipping of a deleterious exon), decreased toxicity, and/or improved distribution to a tissue. In some embodiments, a tissue is muscle tissue. In some embodiments, a tissue is skeletal muscle, gastrocnemius, triceps, heart or diaphragm. In some embodiments, improved properties include reduced hTLR9 agonist activity. In some embodiments, improved properties include hTLR9 antagonist activity. In some embodiments, improved properties include increased hTLR9 antagonist activity. In some embodiments, conjugation of oligonucleotides with lipids can provide hTLR9 antagonist activities, for example, as demonstrated in FIGS. 27 and 28.

### Lipids

In some embodiments, the present disclosure provides a composition comprising a biologically active agent and a lipid. Many lipids can be utilized in provided technologies in accordance with the present disclosure.

In some embodiments, a lipid comprises an R^{LD} group, wherein R^{LD} is an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—, wherein: each R′ is independently —R, —C(O)R, —CO₂R, or —SO₂R, or:
two R′ are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, and heterocyclylene; and
each R is independently hydrogen, or an optionally substituted group selected from C₁-C₆ aliphatic, phenyl, carbocyclyl, aryl, heteroaryl, or heterocyclyl.

In some embodiments, a lipid comprises an R^{LD} group, wherein R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—, wherein:
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, and heterocyclylene; and
each R is independently hydrogen, or an optionally substituted group selected from C₁-C₆ aliphatic, phenyl, carbocyclyl, aryl, heteroaryl, or heterocyclyl.

In some embodiments, a lipid comprises an R^{LD} group, wherein R^{LD} is an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—, wherein:
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, and heterocyclylene; and
each R is independently hydrogen, or an optionally substituted group selected from C₁-C₆ aliphatic, phenyl, carbocyclyl, aryl, heteroaryl, or heterocyclyl.

In some embodiments, R^{LD} is an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is a hydrocarbon group consisting carbon and hydrogen atoms.

In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is a hydrocarbon group consisting carbon and hydrogen atoms.

In some embodiments, R^{LD} is an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and -Cy-. In some embodiments, R^{LD} is a hydrocarbon goup consisting carbon and hydrogen atoms.

The aliphatic group of R^{LD} can be a variety of suitable length. In some embodiments, it is C₁₀-C₈₀. In some embodiments, it is C₁₀-C₇₅. In some embodiments, it is C₁₀-C₇₀. In some embodiments, it is C₁₀-C₆₅. In some embodiments, it is C₁₀-C₆₀. In some embodiments, it is C₁₀-C₅₀. In some embodiments, it is C₁₀-C₄₀. In some embodiments, it is C₁₀-C₃₅. In some embodiments, it is C₁₀-C₃₀. In some embodiments, it is C₁₀-C₂₅. In some embodiments, it is C₁₀-C₂₄. In some embodiments, it is C₁₀-C₂₃. In some embodiments, it is C₁₀-C₂₂. In some embodiments, it is C₁₀-C₂₁. In some embodiments, it is C₁₂-C₂₂. In some embodiments, it is C₁₃-C₂₂. In some embodiments, it is C₁₄-C₂₂. In some embodiments, it is C₁₅-C₂₂. In some embodiments, it is C₁₆-C₂₂. In some embodiments, it is C₁₇-C₂₂. In some embodiments, it is C₁₈-C₂₂. In some embodiments, it is C₁₀-C₂₀. In some embodiments, the lower end of the range is C₁₀, C₁₁, C₁₂, C₁₃, C₁₄, C₁₅, C₁₆, C₁₇, or Cis. In some embodiments, the higher end of the range is C₁₈, C₁₉, C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₅₅, or C₆₀. In some embodiments, it is C₁₀. In some embodiments, it is C₁₁. In some embodiments, it is C₁₂. In some embodiments, it is C₁₃. In some embodiments, it is C₁₄. In some embodiments, it is C₁₅. In some embodiments, it is C₁₆. In some embodiments, it is C₁₇. In some embodiments, it is C₁₈. In some embodiments, it is C₁₉. In some embodiments, it is C₂₀. In some embodiments, it is C₂₁. In some embodiments, it is C₂₂. In some embodiments, it is C₂₃. In some embodiments, it is C₂₄. In some embodiments, it is C₂₅. In some embodiments, it is C₃₀. In some embodiments, it is C₃₅. In some embodiments, it is C₄₀. In some embodiments, it is C₄₅. In some embodiments, it is C₅₀. In some embodiments, it is C₅₅. In some embodiments, it is C₆₀.

In some embodiments, a lipid comprises no more than one R^{LD} group. In some embodiments, a lipid comprises two or more R^{LD} groups.

In some embodiments, a lipid is conjugated to a biologically active agent, optionally through a linker, as a moiety comprising an R^{LD} group. In some embodiments, a lipid is conjugated to a biologically active agent, optionally through a linker, as a moiety comprising no more than one R^{LD} group. In some embodiments, a lipid is conjugated to a biologically active agent, optionally through a linker, as an R^{LD} group. In some embodiments, a lipid is conjugated to a biologically active agent, optionally through a linker, as a moiety comprising two or more R^{LD} groups.

In some embodiments, R^{LD} is an optionally substituted, C₁₀-C₄₀ saturated or partially unsaturated, aliphatic chain. In some embodiments, a lipid comprises an optionally substituted C₁₀-C₄₀ saturated or partially unsaturated, aliphatic chain.

In some embodiments, R^{LD} is an optionally substituted C₁₀-C₄₀ linear, saturated or partially unsaturated, aliphatic chain. In some embodiments, a lipid comprises an optionally substituted C₁₀-C₄₀ linear, saturated or partially unsaturated, aliphatic chain.

In some embodiments, R^{LD} is a C₁₀-C₄₀ linear, saturated or partially unsaturated, aliphatic chain, optionally substituted with one or more C₁₋₄ aliphatic groups. In some embodiments, a lipid comprises a C₁₀-C₄₀ linear, saturated or partially unsaturated, aliphatic chain, optionally substituted with one or more C₁₋₄ aliphatic groups. In some embodiments, R^{LD} is a C₁₀-C₄₀ linear, saturated or partially unsaturated, aliphatic chain, optionally substituted with one or more C₁₋₂ aliphatic groups. In some embodiments, a lipid comprises a C₁₀-C₄₀ linear, saturated or partially unsaturated, aliphatic chain, optionally substituted with one or more C₁₋₂ aliphatic groups. In some embodiments, R^{LD} is a C₁₀-C₄₀ linear, saturated or partially unsaturated, aliphatic chain, optionally substituted with one or more methyl groups. In some embodiments, a lipid comprises a C₁₀-C₄₀ linear, saturated or partially unsaturated, aliphatic chain, optionally substituted with one or more methyl groups.

In some embodiments, R^{LD} is an unsubstituted C₁₀-C₄₀ linear, saturated or partially unsaturated, aliphatic chain. In some embodiments, a lipid comprises an unsubstituted C₁₀-C₄₀ linear, saturated or partially unsaturated, aliphatic chain.

In some embodiments, a lipid comprises no more than one optionally substituted C₁₀-C₄₀ linear, saturated or partially unsaturated, aliphatic chain. In some embodiments, a lipid comprises two or more optionally substituted C₁₀-C₄₀ linear, saturated or partially unsaturated, aliphatic chain.

In some embodiments, R^{LD} is an optionally substituted, C₁₀-C₆₀ saturated or partially unsaturated, aliphatic chain. In some embodiments, a lipid comprises an optionally substituted C₁₀-C₆₀ saturated or partially unsaturated, aliphatic chain.

In some embodiments, R^{LD} is an optionally substituted C₁₀-C₆₀ linear, saturated or partially unsaturated, aliphatic chain. In some embodiments, a lipid comprises an optionally substituted C₁₀-C₆₀ linear, saturated or partially unsaturated, aliphatic chain.

In some embodiments, R^{LD} is a C₁₀-C₆₀ linear, saturated or partially unsaturated, aliphatic chain, optionally substituted with one or more C₁₋₄ aliphatic groups. In some embodiments, a lipid comprises a C₁₀-C₆₀ linear, saturated or partially unsaturated, aliphatic chain, optionally substituted with one or more C₁₋₄ aliphatic groups. In some embodiments, R^{LD} is a C₁₀-C₆₀ linear, saturated or partially unsaturated, aliphatic chain, optionally substituted with one or more C₁₋₂ aliphatic groups. In some embodiments, a lipid comprises a C₁₀-C₆₀ linear, saturated or partially unsaturated, aliphatic chain, optionally substituted with one or more C₁₋₂ aliphatic groups. In some embodiments, R^{LD} is a C₁₀-C₆₀ linear, saturated or partially unsaturated, aliphatic chain, optionally substituted with one or more methyl groups. In some embodiments, a lipid comprises a C₁₀-C₆₀ linear, saturated or partially unsaturated, aliphatic chain, optionally substituted with one or more methyl groups.

In some embodiments, R^{LD} is an unsubstituted C₁₀-C₆₀ linear, saturated or partially unsaturated, aliphatic chain. In some embodiments, a lipid comprises an unsubstituted C₁₀-C₆₀ linear, saturated or partially unsaturated, aliphatic chain.

In some embodiments, a lipid comprises no more than one optionally substituted C₁₀-C₆₀ linear, saturated or partially unsaturated, aliphatic chain. In some embodiments, a lipid comprises two or more optionally substituted C₁₀-C₆₀ linear, saturated or partially unsaturated, aliphatic chain.

In some embodiments, R^{LD} is an optionally substituted, C₁₀-C₈₀ saturated or partially unsaturated, aliphatic chain. In some embodiments, a lipid comprises an optionally substituted C₁₀-C₈₀ saturated or partially unsaturated, aliphatic chain.

In some embodiments, R^{LD} is an optionally substituted C₁₀-C₈₀ linear, saturated or partially unsaturated, aliphatic chain. In some embodiments, a lipid comprises an optionally substituted C₁₀-C₈₀ linear, saturated or partially unsaturated, aliphatic chain.

In some embodiments, R^{LD} is a C₁₀-C₈₀ linear, saturated or partially unsaturated, aliphatic chain, optionally substituted with one or more C₁₋₄ aliphatic groups. In some embodiments, a lipid comprises a C₁₀-C₈₀ linear, saturated or partially unsaturated, aliphatic chain, optionally substituted with one or more C₁₋₄ aliphatic groups. In some embodiments, R^{LD} is a C₁₀-C₈₀ linear, saturated or partially unsaturated, aliphatic chain, optionally substituted with one or more C₁₋₂ aliphatic groups. In some embodiments, a lipid comprises a C₁₀-C₈₀ linear, saturated or partially unsaturated, aliphatic chain, optionally substituted with one or more C₁₋₂ aliphatic groups. In some embodiments, R^{LD} is a C₁₀-C₈₀ linear, saturated or partially unsaturated, aliphatic chain, optionally substituted with one or more methyl groups. In some embodiments, a lipid comprises a C₁₀-C₈₀ linear, saturated or partially unsaturated, aliphatic chain, optionally substituted with one or more methyl groups.

In some embodiments, R^{LD} is an unsubstituted C₁₀-C₈₀ linear, saturated or partially unsaturated, aliphatic chain. In some embodiments, a lipid comprises an unsubstituted C₁₀-C₈₀ linear, saturated or partially unsaturated, aliphatic chain.

In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain.

In some embodiments, R^{LD} is or comprises a C₁₀ saturated linear aliphatic chain. In some embodiments, R^{LD} is or comprises a C₁₀ partially unsaturated linear aliphatic chain. In some embodiments, R^{LD} is or comprises a C₁₁ saturated linear aliphatic chain. In some embodiments, R^{LD} is or comprises a C₁₁ partially unsaturated linear aliphatic chain. In some embodiments, R^{LD} is or comprises a C₁₂ saturated linear aliphatic chain. In some embodiments, R^{LD} is or comprises a C₁₂ partially unsaturated linear aliphatic chain. In some embodiments, R^{LD} is or comprises a C₁₃ saturated linear aliphatic chain. In some embodiments, R^{LD} is or comprises a C₁₃ partially unsaturated linear aliphatic chain. In some embodiments, R^{LD} is or comprises a C₁₄ saturated linear aliphatic chain. In some embodiments, R^{LD} is or comprises a C₁₄ partially unsaturated linear aliphatic chain. In some embodiments, R^{LD} is or comprises a C₁₅ saturated linear aliphatic chain. In some embodiments, R^{LD} is or comprises a C₁₅ partially unsaturated linear aliphatic chain. In some embodiments, R^{LD} is or comprises a C₁₆ saturated linear aliphatic chain. In some embodiments, R^{LD} is or comprises a C₁₆ partially unsaturated linear aliphatic chain. In some embodiments, R^{LD} is or comprises a C₁₇ saturated linear aliphatic chain. In some embodiments, R^{LD} is or comprises a C₁₇ partially unsaturated linear aliphatic chain. In some embodiments, R^{LD} is or comprises a C₁₈ saturated linear aliphatic chain. In some embodiments, R^{LD} is or comprises a C₁₈ partially unsaturated linear aliphatic chain. In some embodiments, R^{LD} is or comprises a C₁₉ saturated linear aliphatic chain. In some embodiments, R^{LD} is or comprises a C₁₉ partially unsaturated linear aliphatic chain. In some embodiments, R^{LD} is or comprises a C₂₀ saturated linear aliphatic chain. In some embodiments, R^{LD} is or comprises a C₂₀ partially unsaturated linear aliphatic chain. In some embodiments, R^{LD} is or comprises a C₂₁ saturated linear aliphatic chain. In some embodiments, R^{LD} is or comprises a C₂₁ partially unsaturated linear aliphatic chain. In some embodiments, R^{LD} is or comprises a C₂₂ saturated linear aliphatic chain. In some embodiments, R^{LD} is or comprises a C₂₂ partially unsaturated linear aliphatic chain. In some embodiments, R^{LD} is or comprises a C₂₃ saturated linear aliphatic chain. In some embodiments, R^{LD} is or comprises a C₂₃ partially unsaturated linear aliphatic chain. In some embodiments, R^{LD} is or comprises a C₂₄ saturated linear aliphatic chain. In some embodiments, R^{LD} is or comprises a C₂₄ partially unsaturated linear aliphatic chain. In some embodiments, R^{LD} is or comprises a C₂₅ saturated linear aliphatic chain. In some embodiments, R^{LD} is or comprises a C₂₅ partially unsaturated linear aliphatic chain. In some embodiments, R^{LD} is or comprises a C₂₆ saturated linear aliphatic chain. In some embodiments, R^{LD} is or comprises a C₂₆ partially unsaturated linear aliphatic chain. In some embodiments, R^{LD} is or comprises a C₂₇ saturated linear aliphatic chain. In some embodiments, R^{LD} is or comprises a C₂₇ partially unsaturated linear aliphatic chain. In some embodiments, R^{LD} is or comprises a C₂₈ saturated linear aliphatic chain. In some embodiments, R^{LD} is or comprises a C₂₈ partially unsaturated linear aliphatic chain. In some embodiments, R^{LD} is or comprises a C₂₉ saturated linear aliphatic chain. In some embodiments, R^{LD} is or comprises a C₂₉ partially unsaturated linear aliphatic chain. In some embodiments, R^{LD} is or comprises a C₃₀ saturated linear aliphatic chain. In some embodiments, R^{LD} is or comprises a C₃₀ partially unsaturated linear aliphatic chain.

In some embodiments, a lipid has the structure of R^{LD}—OH. In some embodiments, a lipid has the structure of R^{LD}—C(O)OH. In some embodiments, R^{LD} is

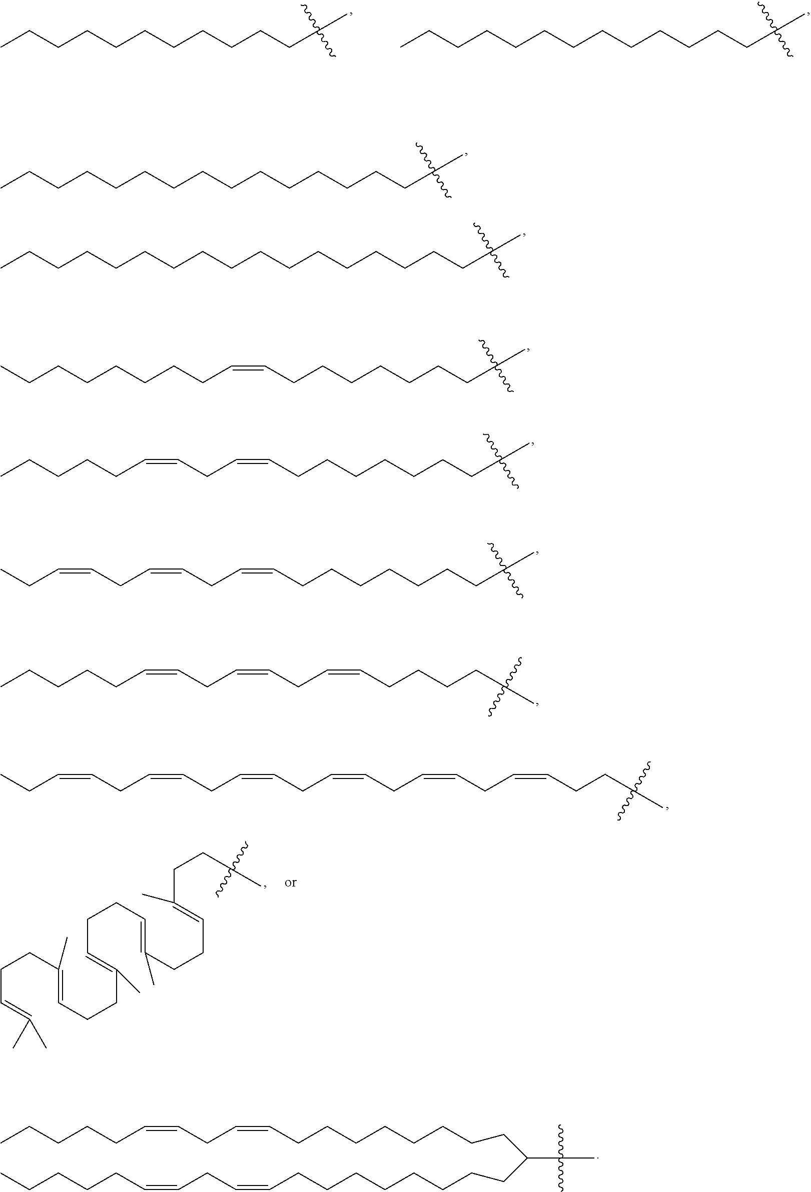

Example oligonucleotides comprising such RD groups are illustrated, e.g., in Table 4A. In some embodiments, a lipid is lauric acid, myristic acid, palmitic acid, stearic acid, oleic acid, linoleic acid, alpha-linolenic acid, gamma-linolenic acid, docosahexaenoic acid (DHA or cis-DHA), turbinaric acid, arachidonic acid, and dilinoleyl. In some embodiments, a lipid has a structure of:

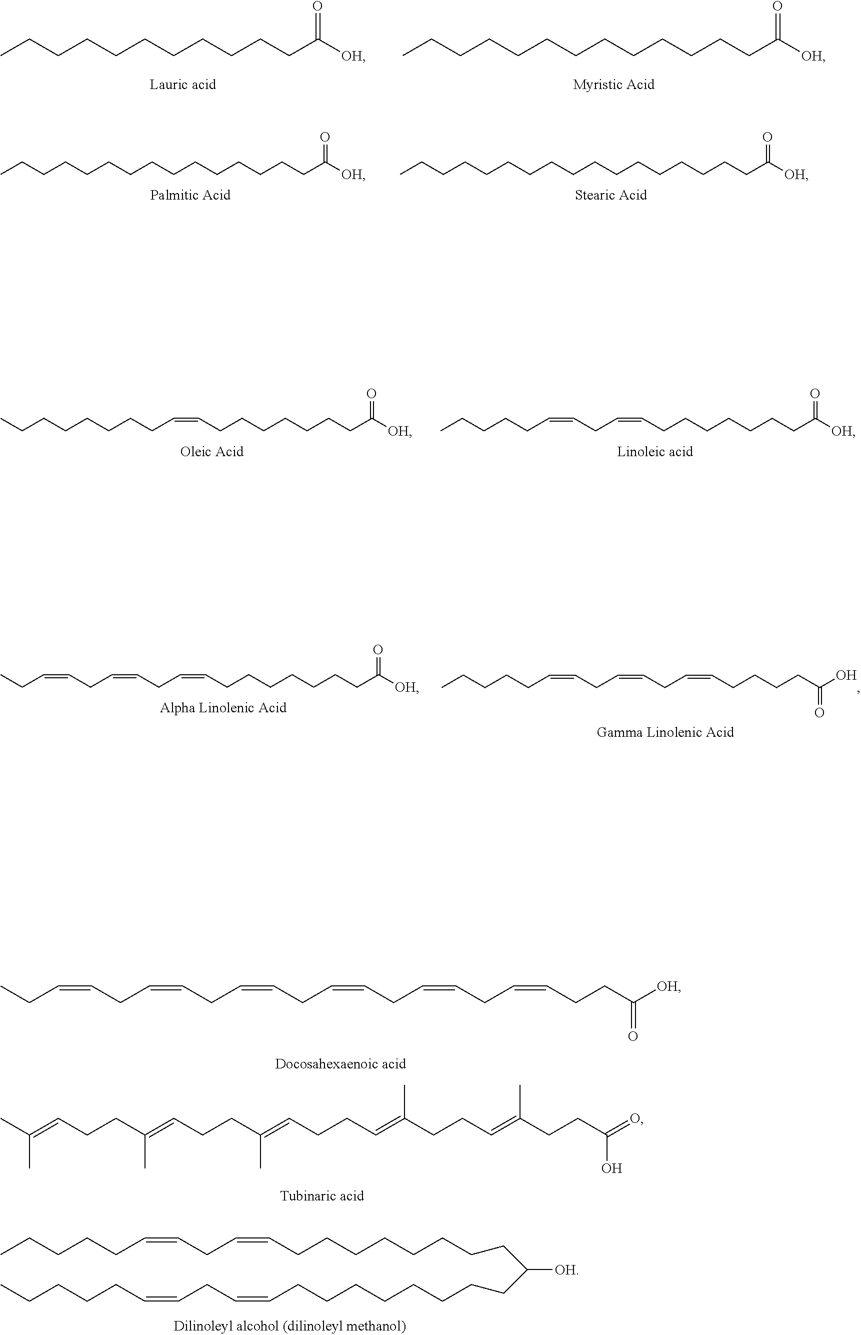

Example oligonucleotides comprising conjugation with these lipids are illustrated, e.g., in Table 4.

In some embodiments, a lipid is, comprises or consists of any of: an at least partially hydrophobic or amphiphilic molecule, a phospholipid, a triglyceride, a diglyceride, a monoglyceride, a fat-soluble vitamin, a sterol, a fat and a wax. In some embodiments, a lipid is any of: a fatty acid, glycerolipid, glycerophospholipid, sphingolipid, sterol lipid, prenol lipid, saccharolipid, polyketide, and other molecule.

In some embodiments, a lipid is conjugated to a biologically active agent optionally through a linker moiety. A person having ordinary skill in the art appreciates that various technologies can be utilized to conjugate lipids to biologically active agent in accordance with the present disclosure. For example, for lipids comprising carboxyl groups, such lipids can be conjugated through the carboxyl groups.

Lipids can be conjugated to oligonucleotides optionally through linkers. Various types of linkers in the art can be utilized in accordance of the present disclosure. In some embodiments, a linker comprise a phosphate group, which can, for example, be used for conjugating lipids through chemistry similar to those employed in oligonucleotide synthesis. In some embodiments, a linker comprises an amide, ester, or ether group.

In some embodiments, a linker has the structure of -L^{LD}-. In some embodiments, L^{LD} is T^{LD} having the structure of

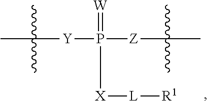

wherein each variable is independently as defined and described. In some embodiments, T^{LD} has the structure of formula I. In some embodiments, T^{LD} with the 5′-O— of an oligonucleotide chain form a phosphorothioate linkage (—OP(O)(S⁻)O—). In some embodiments, T^{LD} with the 5′-O— of an oligonucleotide chain form an Sp phosphorothioate linkage. In some embodiments, T^{LD} with the 5′-O— of an oligonucleotide chain form an Rp phosphorothioate linkage. In some embodiments, T^{LD} with the 5′-O— of an oligonucleotide chain form a phosphate linkage (—OP(O)(O⁻)O—). In some embodiments, T^{LD} with the 5′-O— of an oligonucleotide chain form a phosphorodithioate linkage. In some embodiments, L^{LD} is -L-T^{LD}-. In some embodiments, Y connects to -L- and —Z— is a covalent bond, so that P directly connects to a hydroxyl group of the oligonucleotide chain. In some embodiments, P connects to the 5′-end hydroxyl (5′-O—) to form a phosphate group (natural phosphate linkage) or phosphorothioate group (phosphorothioate linkage). In some embodiments, the phosphorothioate linkage is chirally controlled and can be either Rp or Sp. Unless otherwise specified, chiral centers in the linkers (e.g., P in T^{LD}) can be either stereorandom or chirally controlled, and they are not considered as part of the backbone chiral centers, e.g., for determining whether a composition is chirally controlled. In some embodiments, L^{LD} is —NH—(CH₂)₆-T^{LD}-. In some embodiments, L^{LD} is —C(O)—NH—(CH₂)₆-T^{LD}-.

In some embodiments, a linker has the structure of -L-. In some embodiments, after conjugation to oligonucleotides, a lipid forms a moiety having the structure of -L-R^{LD}wherein each of L and R^{LD} is independently as defined and described herein.

In some embodiments, -L- comprises a bivalent aliphatic chain. In some embodiments, -L- comprises a phosphate group. In some embodiments, -L- comprises a phosphorothioate group. In some embodiments, -L- has the structure of —C(O)NH—(CH₂)₆—OP(═O)(S⁻)—. In some embodiments, -L- has the structure of —C(O)NH—(CH₂)₆—OP(═O)(O⁻)—.

Lipids, optionally through linkers, can be conjugated to oligonucleotides at various suitable locations. In some embodiments, lipids are conjugated through the 5′-OH group. In some embodiments, lipids are conjugated through the 3′-OH group. In some embodiments, lipids are conjugated through one or more sugar moieties. In some embodiments, lipids are conjugated through one or more bases. In some embodiments, lipids are incorporated through one or more internucleotidic linkages. In some embodiments, an oligonucleotide may contain multiple conjugated lipids which are independently conjugated through its 5′-OH, 3′-OH, sugar moieties, base moieties and/or internucleotidic linkages.

In some embodiments, a linker is a moiety that connects two parts of a composition; as a non-limiting example, a linker physically connects a active compound to a lipid. Non-limiting examples of suitable linkers include: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group.

In some embodiments, a lipid is conjugated to an active compound optionally through a linker moiety. A person having ordinary skill in the art appreciates that various technologies can be utilized to conjugate lipids to active compound in accordance with the present disclosure. For example, for lipids comprising carboxyl groups, such lipids can be conjugated through the carboxyl groups. In some embodiments, a lipid is conjugated through a linker having the structure of -L-, wherein L is as defined and described in formula I. In some embodiments, L comprises a phosphate diester or modified phosphate diester moiety. In some embodiments, a compound formed by lipid conjugation has the structure of (R^{LD}-L-)ₓ-(active compound), wherein x is 1 or an integer greater than 1, and each of R^{LD} and L is independently as defined and described herein. In some embodiments, x is 1. In some embodiments, x is greater than 1. In some embodiments, x is 1-50. In some embodiments, an active compound is an oligonucleotide. For example, in some embodiments, a conjugate has the following structures:

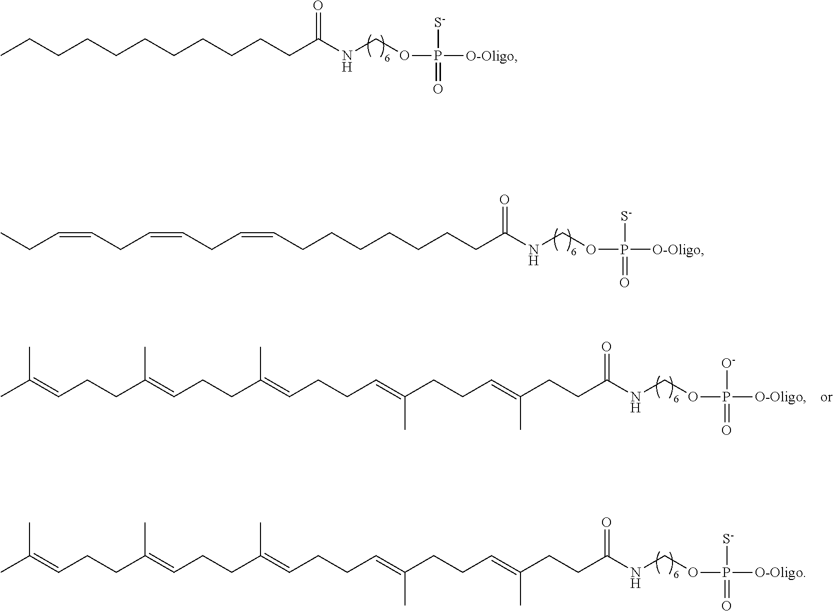

In some embodiments, a linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; and a linker comprising at least one peptide-based cleavage group. Other non-limiting examples of linkers are described herein, or detailed in FIG. 7. In some embodiments, a linker has the structure of -L^{LD}-. In some embodiments, a linker has the structure of -L-. In some embodiments, a linker comprises a linkage of formula I. In some embodiments, a linker is —C(O)NH—(CH₂)₆-L¹-, wherein L¹ has the structure of formula I as described herein. In some embodiments, a linker is —C(O)NH—(CH₂)₆—O—P(═O)(SR¹)—O—. In some embodiments, R¹ is —H, and a linker is —C(O)NH—(CH₂)₆—O—P(═O)(SH)—O—, in some conditions, e.g., certain pH, —C(O)NH—(CH₂)₆—O—P(═O)(S⁻)—O—. In some embodiments, a linker is —C(O)NH—(CH₂)₆—O—P(═S)(SR¹)—O—. In some embodiments, R¹ is —H, and a linker is —C(O)NH—(CH₂)₆—O—P(═S)(SH)—O—, in some conditions, e.g., certain pH, —C(O)NH—(CH₂)₆—O—P(═S)(S⁻)—O—. In some embodiments, a linker is —C(O)NH—(CH₂)₆—O—P(═S)(OR¹)—O—, wherein R¹ is —CH₂CH₂CN. In some embodiments, a linker is —C(O)NH—(CH₂)₆—O—P(═S)(SR′)—O—, wherein R¹ is —CH₂CH₂CN. In some embodiments, a provided oligonucleotide is coupled with a linker and forms a structure of H-linker-oligonucleotide. In some embodiments, a provided oligonucleotide is conjugated to a lipid and forms the structure of lipid-linker-oligonucleotide, e.g., R^{LD}-L^{L}D-oligonucleotide. In some embodiments, the —O— end of a linker is connected to an oligonucleotide. In some embodiments, the —O— end of a linker is connected to the 5′-end oligonucleotide (—O— being the oxygen in the 5′-OH).

In some embodiments, a linker comprises a PO (phosphodiester linkage), a PS (phosphorothioate linkage) or PS2 (phosphorodithioate linkage). A non-limiting example including a PS linker is shown below. In some embodiments, a linker is —O—P(O)(OH)—O— [phosphodiester], —O—P(O)(SH)—O— [phosphorothioate] or —O—P(S)(SH)—O— [phosphorodithioate]. In some embodiments, a linker comprises a C₆ amino moiety (—NH—(CH₂)₆—), which is illustrated below. In some embodiments, a linker comprises a C₆ amino bound to a PO, a PS, or PS2. In some embodiments, a linker is a C₆ amino bound to a PO, a PS, or PS2. In some embodiments, a linker, e.g., L^{LD} or L, is —C(O)—NH—(CH₂)₆—P(O)(OH)—. In some embodiments, a linker, e.g., L^{LD} or L, is —C(O)—NH—(CH₂)₆—P(O)(OH)—, wherein —C(O)— is connected to a lipid moiety and —P(O)(OH)— is connected to an oligonucleotide chain. In some embodiments, a linker, e.g., L^{LD} or L, is —C(O)—NH—(CH₂)₆—P(O)(OH)—, wherein —C(O)— is connected to a lipid moiety and —P(O)(OH)— is connected to the 5′-O— of an oligonucleotide chain. In some embodiments, a linker, e.g., L^{LD} or L, is —C(O)—NH—(CH₂)₆—P(O)(OH)—, wherein —C(O)— is connected to a lipid moiety and —P(O)(OH)— is connected to the 3′-O— of an oligonucleotide chain. In some embodiments, a linker, e.g., L^{LD} or L, is —C(O)—NH—(CH₂)₆—P(O)(SH)—. In some embodiments, a linker, e.g., L^{LD} or L, is —C(O)—NH—(CH₂)₆—P(O)(SH)—, wherein —C(O)— is connected to a lipid moiety and —P(O)(SH)— is connected to an oligonucleotide chain. In some embodiments, a linker, e.g., L^{LD} or L, is —C(O)—NH—(CH₂)₆—P(O)(SH)—, wherein —C(O)— is connected to a lipid moiety and —P(O)(SH)— is connected to the 5′-O— of an oligonucleotide chain. In some embodiments, a linker, e.g., L^{LD} or L, is —C(O)—NH—(CH₂)₆—P(O)(SH)—, wherein —C(O)— is connected to a lipid moiety and —P(O)(SH)— is connected to the 3′-O— of an oligonucleotide chain. In some embodiments, a linker, e.g., L^{LD} or L, is —C(O)—NH—(CH₂)₆—P(S)(SH)—. In some embodiments, a linker, e.g., L^{LD} or L, is —C(O)—NH—(CH₂)₆—P(S)(SH)—, wherein —C(O)— is connected to a lipid moiety and —P(S)(SH)— is connected to an oligonucleotide chain. In some embodiments, a linker, e.g., L^{L}D or L, is —C(O)—NH—(CH₂)₆—P(S)(SH)—, wherein —C(O)— is connected to a lipid moiety and —P(S)(SH)— is connected to the 5′-O— of an oligonucleotide chain. In some embodiments, a linker, e.g., L^{L}D or L, is —C(O)—NH—(CH₂)₆—P(S)(SH)—, wherein —C(O)— is connected to a lipid moiety and —P(S)(SH)— is connected to the 3′-O— of an oligonucleotide chain. As appreciated by a person having ordinary skill in the art, at certain pH —P(O)(OH)—, —P(O)(SH)—, —P(S)(SH)— may exist as —P(O)(O⁻)—, —P(O)(S⁻)—, —P(S)(S⁻)—, respectively. In some embodiments, a lipid moiety is R^{LD}.

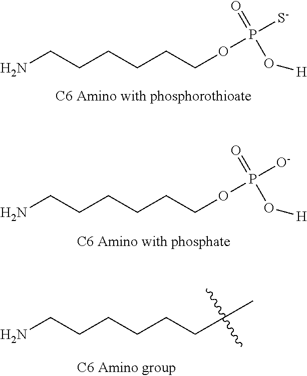 [SMILES: NCCCCCCOP(=O)(O)[S-] ; NCCCCCCOP(=O)([O-])O ; *CCCCCCN]

Various chemistry and linkers can be used for conjugation in accordance with the present disclosure. For example, lipids, targeting components, etc. can be conjugated to oligonucleotides through linkers using chemistry as described below either on solid phase or in solution phase to prepare certain provided oligonucleotides, for example, those described in Table 4 (WV-2538, WV-2733, WV-2734, WV-2578 to WV-2588, WV-2807, WV-2808, WV-3022 to WV-3027, WV-3029 to WV-3038, WV-3084 to WV-3089, WV-3357 to WV-3366, WV-3517, WV-3520, WV-3543 to WV-3560, WV-3753, WV-3754, WV-3820, WV-3821, WV-3855, WV-3856, WV-3976, WV-3977, WV-3979, WV-3980, WV-4106, WV-4107, etc.):

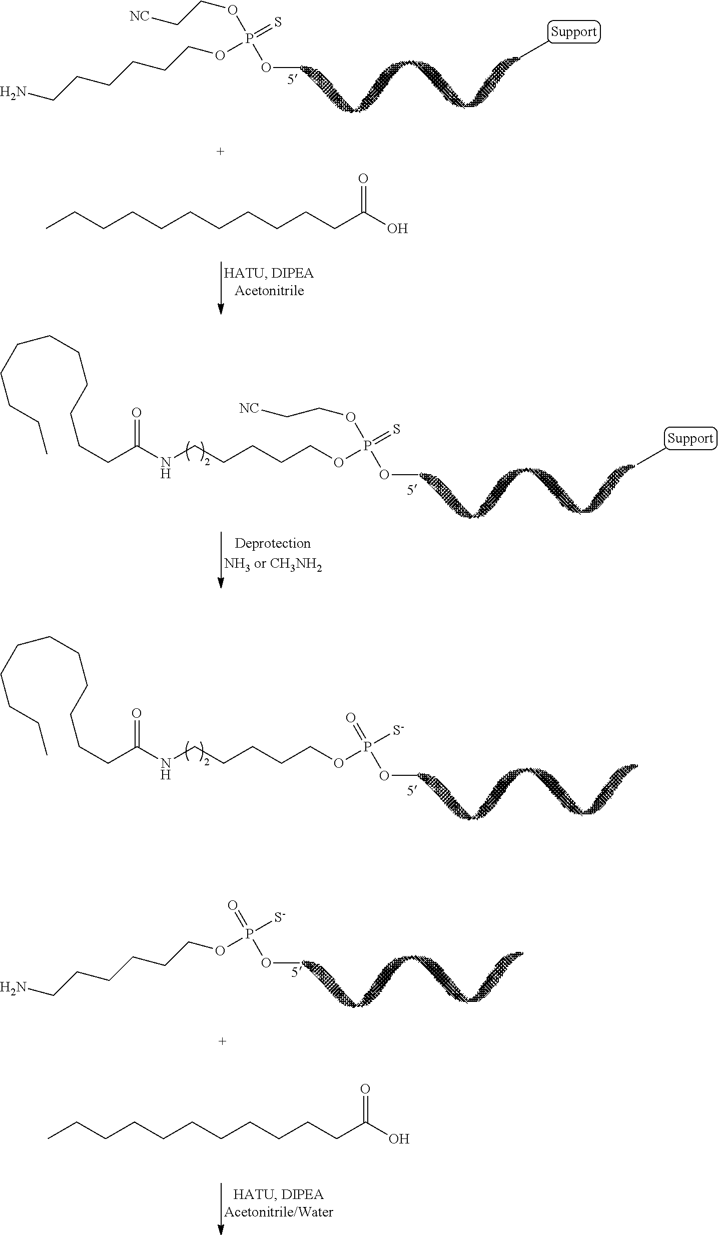

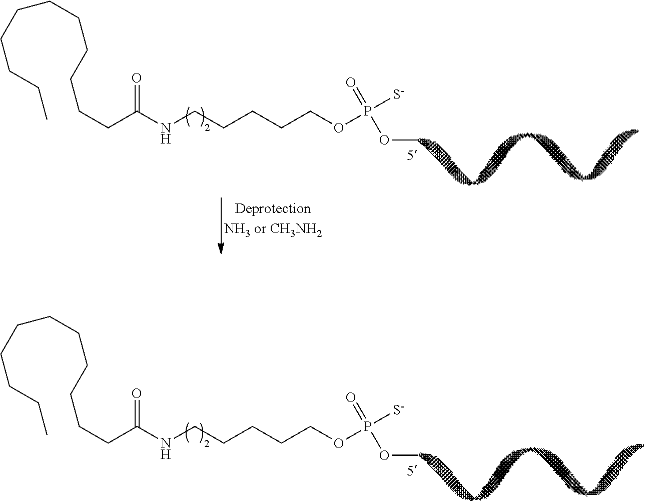

Non-limiting examples of protocols for conjugation of a lipid to a biologically active agent (e.g., an oligonucleotide) using a linker are described, e.g., in the Examples.

In some embodiments, a lipid is not conjugated to a biologically active agent.

### Biologically Active Agents

Various biologically active agents can be effectively delivered to their targets in accordance with the present disclosure. In some embodiments, a biologically active agent is selected from the group consisting of: a small molecule, a peptide, a protein, a component of a CRISPR-Cas system, a carbohydrate, a therapeutic agent, a chemotherapeutic agent, a vaccine, a nucleic acid, and a lipid. In some embodiments, a nucleic acid is an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, a vector, or a portion thereof.

In some embodiments, a biologically active agent is a small molecule. In some embodiments, a biologically active agent is selected from biologics. In some embodiments, a biologically active agent is a protein. In some embodiments, a biologically active agent is an antibody. In some embodiments, a biologically active agent is a peptide.

In some embodiments, a biologically active agent is an oligonucleotide. In some embodiments, the present disclosure provides compositions comprising an oligonucleotide and a lipid. Among other things, such compositions are surprisingly effective at delivering oligonucleotides to their target locations, in some embodiments, delivering oligonucleotides into the cells at the target locations. In some embodiments, provided technologies are surprisingly effective at delivering oligonucleotides to muscle cells, tissues, etc. As will be appreciated by a person having ordinary skill in the art, oligonucleotides of various sequences, functions, etc., can be included in provided technologies and can be efficiently and effectively delivered to target locations, including into cells, in accordance with the present disclosure.

In some embodiments, provided technologies can be utilized to effectively improve delivery of oligonucleotides to their target location(s) in a subject, e.g., in a mammal or human subject, etc. In some embodiments, provided technologies provide surprising achievement of efficient and/or effective delivery of oligonucleotide(s) into cells (i.e., to intracellular location(s) such as cytoplasm, nucleus, etc.) of a subject.

In some embodiments, provided technologies permit or facilitate delivery of an effective and/or desired amount of oligonucleotide to its target location(s) so that, for example, a comparable or higher level of the oligonucleotide is achieved at the target location(s) than is observed when the oligonucleotide is administered absent the lipid, in some embodiments, even though a lower amount of the oligonucleotide may be administered with the lipid than without. In some embodiments, provided technologies permit or facilitate improved distribution (i.e., increased relative level of oligonucleotide at a target location(s) as compared with at a non-target location(s)) relative to an appropriate control (e.g., that level observed when the oligonucleotide is comparably administered absent the lipid). In some embodiments, provided technologies render oligonucleotides that have otherwise been considered unsuitable for therapeutic use to be successfully used for treating various diseases, disorders and/or conditions.

In some embodiments, provided technologies are particularly effective at delivering oligonucleotides to particular types of cells and tissues, including, but not limited to, cells and tissues outside the liver (e.g., extra-hepatic), including, but not limited to, muscle cells and tissues. In some embodiments, the present disclosure provides technologies that are surprisingly effective at delivering oligonucleotides to muscle cells and tissues, e.g., of gastrocnemius, heart, quadriceps, triceps, and/or thoracic diaphragm, etc. In some embodiments, provided technologies effectively deliver an oligonucleotide into cells of gastrocnemius muscle of a subject. In some embodiments, provided technologies effectively deliver an oligonucleotide into cells of cardiac muscle of a subject. In some embodiments, provided technologies effectively deliver an oligonucleotide into cells of quadriceps of a subject. In some embodiments, provided technologies effectively deliver an oligonucleotide into cells of thoracic diaphragm of a subject.

In some embodiments, a provided composition is an oligonucleotide composition comprising one ore more lipids, and a plurality of oligonucleotides, which share:
1) a common base sequence;
2) a common pattern of backbone linkages; and
3) a common pattern of backbone phosphorus modifications;
   wherein one or more oligonucleotides of the plurality are independently and optionally conjugated to the lipids.

In some embodiments, a provided composition is an oligonucleotide composition comprising a plurality of oligonucleotides, which share:
1) a common base sequence;
2) a common pattern of backbone linkages; and
3) a common pattern of backbone phosphorus modifications;
   wherein one or more oligonucleotides of the plurality are independently conjugated to one or more lipids.

In some embodiments, a provided composition is a chirally controlled oligonucleotide composition comprising one or more lipids, and a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages, and level of the plurality of oligonucleotides in the composition is pre-determined;

one or more oligonucleotides of the plurality are optionally and independently conjugated to one ore more lipids; and one or more oligonucleotides of the plurality are optionally and independently conjugated to a target component.

In some embodiments, a provided composition is a chirally controlled oligonucleotide composition comprising a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages, and level of the plurality of oligonucleotides in the composition is pre-determined;

one or more oligonucleotides of the plurality are independently conjugated to one or more lipids; and one or more oligonucleotides of the plurality are optionally and independently conjugated to a target component.

In some embodiments, a provided composition is a chirally controlled oligonucleotide composition comprising one or more lipids, and a plurality of oligonucleotides, which share:
1) a common base sequence;
2) a common pattern of backbone linkages; and
3) a common pattern of backbone phosphorus modifications;
   wherein:

the composition is chirally controlled in that the plurality of oligonucleotides share the same stereochemistry at one or more chiral internucleotidic linkages, and level of the plurality of oligonucleotides in the composition is pre-determined;

one or more oligonucleotides of the plurality are optionally and independently conjugated to one ore more lipids; and one or more oligonucleotides of the plurality are optionally and independently conjugated to a target component.

In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, a nucleic acid or oligonucleotide or other biologically active agent is capable of reducing the level and/or activity of a mutant form of any of: dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, a nucleic acid or oligonucleotide or other biologically active agent is capable of increasing the level and/or activity of a wild-type and/or functional form of any of: dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).

In some embodiments, stereochemistry at one or more chiral internucleotidic linkages are the same (chirally controlled). In some embodiments, two or more chiral internucleotidic linkages are chirally controlled. In some embodiments, three or more chiral internucleotidic linkages are chirally controlled. In some embodiments, four or more chiral internucleotidic linkages are chirally controlled. In some embodiments, five or more chiral internucleotidic linkages are chirally controlled. In some embodiments, six or more chiral internucleotidic linkages are chirally controlled. In some embodiments, seven or more chiral internucleotidic linkages are chirally controlled. In some embodiments, eight or more chiral internucleotidic linkages are chirally controlled. In some embodiments, nine or more chiral internucleotidic linkages are chirally controlled. In some embodiments, ten or more chiral internucleotidic linkages are chirally controlled. In some embodiments, 11 or more chiral internucleotidic linkages are chirally controlled. In some embodiments, 12 or more chiral internucleotidic linkages are chirally controlled. In some embodiments, 13 or more chiral internucleotidic linkages are chirally controlled. In some embodiments, 14 or more chiral internucleotidic linkages are chirally controlled. In some embodiments, 15 or more chiral internucleotidic linkages are chirally controlled. In some embodiments, 16 or more chiral internucleotidic linkages are chirally controlled. In some embodiments, 17 or more chiral internucleotidic linkages are chirally controlled. In some embodiments, 18 or more chiral internucleotidic linkages are chirally controlled. In some embodiments, 19 or more chiral internucleotidic linkages are chirally controlled. In some embodiments, 20 or more chiral internucleotidic linkages are chirally controlled. In some embodiments, 21 or more chiral internucleotidic linkages are chirally controlled. In some embodiments, 22 or more chiral internucleotidic linkages are chirally controlled. In some embodiments, 23 or more chiral internucleotidic linkages are chirally controlled. In some embodiments, 24 or more chiral internucleotidic linkages are chirally controlled. In some embodiments, 25 or more chiral internucleotidic linkages are chirally controlled. In some embodiments, 26 or more chiral internucleotidic linkages are chirally controlled. In some embodiments, 27 or more chiral internucleotidic linkages are chirally controlled. In some embodiments, 28 or more chiral internucleotidic linkages are chirally controlled. In some embodiments, 29 or more chiral internucleotidic linkages are chirally controlled. In some embodiments, 30 or more chiral internucleotidic linkages are chirally controlled. In some embodiments, each chiral internucleotidic linkage is chirally controlled, and oligonucleotides share a common pattern of backbone chiral centers.

In some embodiments, not all chiral internucleotidic linkages are chirally controlled, and a chirally controlled oligonucleotide composition is a partially chirally controlled oligonucleotide composition. In some embodiments, all chiral internucleotidic linkage are chirally controlled, and a chirally controlled oligonucleotide composition is a complete chirally controlled oligonucleotide composition.

In some embodiments, a chiral internucleoside linkage is a phosphorothioate. In some embodiments, a phosphorothioate can exist in a Rp or Sp conformation. Various other internucleotidic linkages, which can be chiral, are described herein.

In some embodiments, an oligonucleotide is an oligonucleotide described in Patent Application Publications US20120316224, US20140194610, US20150211006, and WO2015107425, the oligonucleotides and oligonucleotide compositions of each of which are incorporated herein by reference.

In some embodiments, the sequence of the oligonucleotide in the oligonucleotide composition comprises or consists of the sequence of any oligonucleotide described herein. In some embodiments, the sequence of the oligonucleotide in the oligonucleotide composition comprises or consists of the sequence of any oligonucleotide listed in Table 4A. In some embodiments, the oligonucleotide in the oligonucleotide composition is a splice-switching oligonucleotide. In some embodiments, the oligonucleotide in the oligonucleotide composition is capable of skipping or mediating skipping of an exon in the dystrophin gene. In some embodiments, the oligonucleotide in the oligonucleotide composition is a splice-switching oligonucleotide. In some embodiments, the oligonucleotide in the oligonucleotide composition is capable of skipping or mediating skipping of exon 51 in the dystrophin gene. In some embodiments, a biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of exon 51, 45, 53 or 44 in the dystrophin gene. In some embodiments, the sequence of the oligonucleotide in the oligonucleotide composition comprises or consists of the sequence of WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2531, WV-2533, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546.

In some embodiments, structural elements of an oligonucleotide includes any one or more of: base sequence (including length), pattern of chemical modifications to sugar and base moieties, pattern of backbone linkages (e.g., pattern of natural phosphate linkages, phosphorothioate linkages, phosphorothioate triester linkages, and combinations thereof), pattern of backbone chiral centers (e.g., pattern of stereochemistry (Rp/Sp) of chiral internucleotidic linkages), and pattern of backbone phosphorus modifications (e.g., pattern of modifications on the internucleotidic phosphorus atom, such as —S—, and -L-R¹ of formula I). In some embodiments, structural elements include lipid moieties and/or targeting components, for example, as moieties connected to sugars, bases, and/or internucleotidic linkages. In some embodiments, a structural element is base sequence. In some embodiments, a structural element is pattern of chemical modifications. In some embodiments, a structural element is pattern of sugar modifications. In some embodiments, a structural element is nucleobase modifications. In some embodiments, a structural element is pattern of lipid moieties. In some embodiments, a structural element is pattern of targeting component. In some embodiments, a structural element is a linker connecting a biologically active agent, e.g., a provided oligonucleotide, and a lipid moiety and/or a targeting component. In some embodiments, a structural element is pattern of backbone linkages. In some embodiments, a structural element is pattern of backbone chiral centers. In some embodiments, a structural element is pattern of backbone phosphorus modifications. In some embodiments, an oligonucleotide or oligonucleotide composition of any structural elements of any oligonucleotide listed herein can be used in combination with any composition and/or method described herein, including, but not limited to, any combination with any lipid described herein, any additional component described herein, or any other composition (or component thereof) or method described herein. In some embodiments, structural elements of provided oligonucleotides comprise or consist of one or more structural elements of any oligonucleotides described herein. In some embodiments, structural elements of provided oligonucleotides comprise or consist of one or more structural elements of any oligonucleotides listed in Table 4A. In some embodiments, a provided oligonucleotide in a provided oligonucleotide composition is a splice-switching oligonucleotide. In some embodiments, a provided oligonucleotide in a provided oligonucleotide composition is capable of skipping or mediating skipping of an exon in the dystrophin gene. In some embodiments, a provided oligonucleotide in a provided oligonucleotide composition is a splice-switching oligonucleotide. In some embodiments, a provided oligonucleotide in a provided oligonucleotide composition is capable of skipping or mediating skipping of exon 51 in the dystrophin gene. In some embodiments, a biologically active agent comprises or consists of or is a provided oligonucleotide, wherein structural elements of the oligonucleotide comprises or consists of one or more structural elements of an oligonucleotide capable of skipping or mediating skipping of exon 51, 45, 53 or 44 in the dystrophin gene. In some embodiments, one or more structural elements of provided oligonucleotides comprise or consist of one or more structural elements of WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2531, WV-2533, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546. For example, in some embodiments, a structural element is base sequence comprising or consisting of the base sequence of WV-887; in some embodiments, a structural element is pattern of chemical modifications comprising or consisting of that of WV-887; in some embodiments, a structural element is pattern of sugar modifications comprising or consisting of that of WV-887; in some embodiments, a structural element is nucleobase modifications comprising or consisting of that of WV-887; in some embodiments, a structural element is pattern of lipid moieties comprising or consisting of that of WV-3546; in some embodiments, a structural element is pattern of targeting component comprising or consisting of that of WV-3548; in some embodiments, a structural element is a linker comprising or consisting of that of WV-3548; in some embodiments, a structural element is pattern of backbone linkages comprising or consisting of that of WV-887; in some embodiments, a structural element is pattern of backbone chiral centers comprising or consisting of that of WV-887; in some embodiments, a structural element is pattern of backbone phosphorus modifications comprising of consisting of that of WV-887. In some embodiments, the present disclosure provides a chirally controlled oligonucleotide composition, wherein one or more of the structural elements of the oligonucleotide comprise one or more structural elements of WV-2444.

In some embodiments, the present disclosure provides a chirally controlled oligonucleotide composition, wherein one or more of the structural elements of the oligonucleotide comprise one or more structural elements of WV-2445.

In some embodiments, the present disclosure provides a chirally controlled oligonucleotide composition, wherein one or more of the structural elements of the oligonucleotide comprise one or more structural elements of WV-2526.

In some embodiments, the present disclosure provides a chirally controlled oligonucleotide composition, wherein one or more of the structural elements of the oligonucleotide comprise one or more structural elements of WV-2527.

In some embodiments, the present disclosure provides a chirally controlled oligonucleotide composition, wherein one or more of the structural elements of the oligonucleotide comprise one or more structural elements of WV-2528.

In some embodiments, the present disclosure provides a chirally controlled oligonucleotide composition, wherein one or more of the structural elements of the oligonucleotide comprise one or more structural elements of WV-2530.

In some embodiments, the present disclosure provides a chirally controlled oligonucleotide composition, wherein one or more of the structural elements of the oligonucleotide comprise one or more structural elements of WV-2531.

In some embodiments, the present disclosure provides a chirally controlled oligonucleotide composition, wherein one or more of the structural elements of the oligonucleotide comprise one or more structural elements of WV-2578.

In some embodiments, the present disclosure provides a chirally controlled oligonucleotide composition, wherein one or more of the structural elements of the oligonucleotide comprise one or more structural elements of WV-2580.

In some embodiments, the present disclosure provides a chirally controlled oligonucleotide composition, wherein one or more of the structural elements of the oligonucleotide comprise one or more structural elements of WV-2587.

In some embodiments, the present disclosure provides a chirally controlled oligonucleotide composition, wherein one or more of the structural elements of the oligonucleotide comprise one or more structural elements of WV-3047.

In some embodiments, the present disclosure provides a chirally controlled oligonucleotide composition, wherein one or more of the structural elements of the oligonucleotide comprise one or more structural elements of WV-3152.

In some embodiments, the present disclosure provides a chirally controlled oligonucleotide composition, wherein one or more of the structural elements of the oligonucleotide comprise one or more structural elements of WV-3472.

In some embodiments, the present disclosure provides a chirally controlled oligonucleotide composition, wherein one or more of the structural elements of the oligonucleotide comprise one or more structural elements of WV-3473.

In some embodiments, the present disclosure provides a chirally controlled oligonucleotide composition, wherein one or more of the structural elements of the oligonucleotide comprise one or more structural elements of WV-3507.

In some embodiments, the present disclosure provides a chirally controlled oligonucleotide composition, wherein one or more of the structural elements of the oligonucleotide comprise one or more structural elements of WV-3508.

In some embodiments, the present disclosure provides a chirally controlled oligonucleotide composition, wherein one or more of the structural elements of the oligonucleotide comprise one or more structural elements of WV-3509.

In some embodiments, the present disclosure provides a chirally controlled oligonucleotide composition, wherein one or more of the structural elements of the oligonucleotide comprise one or more structural elements of WV-3510.

In some embodiments, the present disclosure provides a chirally controlled oligonucleotide composition, wherein one or more of the structural elements of the oligonucleotide comprise one or more structural elements of WV-3511.

In some embodiments, the present disclosure provides a chirally controlled oligonucleotide composition, wherein one or more of the structural elements of the oligonucleotide comprise one or more structural elements of WV-3512.

In some embodiments, the present disclosure provides a chirally controlled oligonucleotide composition, wherein one or more of the structural elements of the oligonucleotide comprise one or more structural elements of WV-3513.

In some embodiments, the present disclosure provides a chirally controlled oligonucleotide composition, wherein one or more of the structural elements of the oligonucleotide comprise one or more structural elements of WV-3514.

In some embodiments, the present disclosure provides a chirally controlled oligonucleotide composition, wherein one or more of the structural elements of the oligonucleotide comprise one or more structural elements of WV-3515.

In some embodiments, the present disclosure provides a chirally controlled oligonucleotide composition, wherein one or more of the structural elements of the oligonucleotide comprise one or more structural elements of WV-3545.

In some embodiments, the present disclosure provides a chirally controlled oligonucleotide composition, wherein one or more of the structural elements of the oligonucleotide comprise one or more structural elements of WV-3546.

In some embodiments, the present disclosure provides a chirally controlled oligonucleotide composition, wherein one or more of the structural elements of the oligonucleotide consist of one or more structural elements of WV-2444.

In some embodiments, the present disclosure provides a chirally controlled oligonucleotide composition, wherein one or more of the structural elements of the oligonucleotide consist of one or more structural elements of WV-2445.

In some embodiments, the present disclosure provides a chirally controlled oligonucleotide composition, wherein one or more of the structural elements of the oligonucleotide consist of one or more structural elements of WV-2526.

In some embodiments, the present disclosure provides a chirally controlled oligonucleotide composition, wherein one or more of the structural elements of the oligonucleotide consist of one or more structural elements of WV-2527.

In some embodiments, the present disclosure provides a chirally controlled oligonucleotide composition, wherein one or more of the structural elements of the oligonucleotide consist of one or more structural elements of WV-2528.

In some embodiments, the present disclosure provides a chirally controlled oligonucleotide composition, wherein one or more of the structural elements of the oligonucleotide consist of one or more structural elements of WV-2530.

In some embodiments, the present disclosure provides a chirally controlled oligonucleotide composition, wherein one or more of the structural elements of the oligonucleotide consist of one or more structural elements of WV-2531.

In some embodiments, the present disclosure provides a chirally controlled oligonucleotide composition, wherein one or more of the structural elements of the oligonucleotide consist of one or more structural elements of WV-2578.

In some embodiments, the present disclosure provides a chirally controlled oligonucleotide composition, wherein one or more of the structural elements of the oligonucleotide consist of one or more structural elements of WV-2580.

In some embodiments, the present disclosure provides a chirally controlled oligonucleotide composition, wherein one or more of the structural elements of the oligonucleotide consist of one or more structural elements of WV-2587.

In some embodiments, the present disclosure provides a chirally controlled oligonucleotide composition, wherein one or more of the structural elements of the oligonucleotide consist of one or more structural elements of WV-3047.

In some embodiments, the present disclosure provides a chirally controlled oligonucleotide composition, wherein one or more of the structural elements of the oligonucleotide consist of one or more structural elements of WV-3152.

In some embodiments, the present disclosure provides a chirally controlled oligonucleotide composition, wherein one or more of the structural elements of the oligonucleotide consist of one or more structural elements of WV-3472.

In some embodiments, the present disclosure provides a chirally controlled oligonucleotide composition, wherein one or more of the structural elements of the oligonucleotide consist of one or more structural elements of WV-3473.

In some embodiments, the present disclosure provides a chirally controlled oligonucleotide composition, wherein one or more of the structural elements of the oligonucleotide consist of one or more structural elements of WV-3507.

In some embodiments, the present disclosure provides a chirally controlled oligonucleotide composition, wherein one or more of the structural elements of the oligonucleotide consist of one or more structural elements of WV-3508.

In some embodiments, the present disclosure provides a chirally controlled oligonucleotide composition, wherein one or more of the structural elements of the oligonucleotide consist of one or more structural elements of WV-3509.

In some embodiments, the present disclosure provides a chirally controlled oligonucleotide composition, wherein one or more of the structural elements of the oligonucleotide consist of one or more structural elements of WV-3510.

In some embodiments, the present disclosure provides a chirally controlled oligonucleotide composition, wherein one or more of the structural elements of the oligonucleotide consist of one or more structural elements of WV-3511.

In some embodiments, the present disclosure provides a chirally controlled oligonucleotide composition, wherein one or more of the structural elements of the oligonucleotide consist of one or more structural elements of WV-3512.

In some embodiments, the present disclosure provides a chirally controlled oligonucleotide composition, wherein one or more of the structural elements of the oligonucleotide consist of one or more structural elements of WV-3513.

In some embodiments, the present disclosure provides a chirally controlled oligonucleotide composition, wherein one or more of the structural elements of the oligonucleotide consist of one or more structural elements of WV-3514.

In some embodiments, the present disclosure provides a chirally controlled oligonucleotide composition, wherein one or more of the structural elements of the oligonucleotide consist of one or more structural elements of WV-3515.

In some embodiments, the present disclosure provides a chirally controlled oligonucleotide composition, wherein one or more of the structural elements of the oligonucleotide consist of one or more structural elements of WV-3545.

In some embodiments, the present disclosure provides a chirally controlled oligonucleotide composition, wherein one or more of the structural elements of the oligonucleotide consist of one or more structural elements of WV-3546.

In some embodiments, the present disclosure provides a chirally controlled oligonucleotide composition, wherein one or more of the structural elements of the oligonucleotide comprise or consist of one or more structural elements of WV-2444, wherein the composition further comprises a lipid.

In some embodiments, the present disclosure provides a chirally controlled oligonucleotide composition, wherein one or more of the structural elements of the oligonucleotide comprise or consist of one or more structural elements of WV-2445, wherein the composition further comprises a lipid.

In some embodiments, the present disclosure provides a chirally controlled oligonucleotide composition, wherein one or more of the structural elements of the oligonucleotide comprise or consist of one or more structural elements of WV-2526, wherein the composition further comprises a lipid.

In some embodiments, the present disclosure provides a chirally controlled oligonucleotide composition, wherein one or more of the structural elements of the oligonucleotide comprise or consist of one or more structural elements of WV-2527, wherein the composition further comprises a lipid.

In some embodiments, the present disclosure provides a chirally controlled oligonucleotide composition, wherein one or more of the structural elements of the oligonucleotide comprise or consist of one or more structural elements of WV-2528, wherein the composition further comprises a lipid.

In some embodiments, the present disclosure provides a chirally controlled oligonucleotide composition, wherein one or more of the structural elements of the oligonucleotide comprise or consist of one or more structural elements of WV-2530, wherein the composition further comprises a lipid.

In some embodiments, the present disclosure provides a chirally controlled oligonucleotide composition, wherein one or more of the structural elements of the oligonucleotide comprise or consist of one or more structural elements of WV-2531, wherein the composition further comprises a lipid.

In some embodiments, the present disclosure provides a chirally controlled oligonucleotide composition, wherein one or more of the structural elements of the oligonucleotide comprise or consist of one or more structural elements of WV-2578, wherein the composition further comprises a lipid.

In some embodiments, the present disclosure provides a chirally controlled oligonucleotide composition, wherein one or more of the structural elements of the oligonucleotide comprise or consist of one or more structural elements of WV-2580, wherein the composition further comprises a lipid.

In some embodiments, the present disclosure provides a chirally controlled oligonucleotide composition, wherein one or more of the structural elements of the oligonucleotide comprise or consist of one or more structural elements of WV-2587, wherein the composition further comprises a lipid.

In some embodiments, the present disclosure provides a chirally controlled oligonucleotide composition, wherein one or more of the structural elements of the oligonucleotide comprise or consist of one or more structural elements of WV-3047, wherein the composition further comprises a lipid.

In some embodiments, the present disclosure provides a chirally controlled oligonucleotide composition, wherein one or more of the structural elements of the oligonucleotide comprise or consist of one or more structural elements of WV-3152, wherein the composition further comprises a lipid.

In some embodiments, the present disclosure provides a chirally controlled oligonucleotide composition, wherein one or more of the structural elements of the oligonucleotide comprise or consist of one or more structural elements of WV-3472, wherein the composition further comprises a lipid.

In some embodiments, the present disclosure provides a chirally controlled oligonucleotide composition, wherein one or more of the structural elements of the oligonucleotide comprise or consist of one or more structural elements of WV-3473, wherein the composition further comprises a lipid.

In some embodiments, the present disclosure provides a chirally controlled oligonucleotide composition, wherein one or more of the structural elements of the oligonucleotide comprise or consist of one or more structural elements of WV-3507, wherein the composition further comprises a lipid.

In some embodiments, the present disclosure provides a chirally controlled oligonucleotide composition, wherein one or more of the structural elements of the oligonucleotide comprise or consist of one or more structural elements of WV-3508, wherein the composition further comprises a lipid.

In some embodiments, the present disclosure provides a chirally controlled oligonucleotide composition, wherein one or more of the structural elements of the oligonucleotide comprise or consist of one or more structural elements of WV-3509, wherein the composition further comprises a lipid.

In some embodiments, the present disclosure provides a chirally controlled oligonucleotide composition, wherein one or more of the structural elements of the oligonucleotide comprise or consist of one or more structural elements of WV-3510, wherein the composition further comprises a lipid.

In some embodiments, the present disclosure provides a chirally controlled oligonucleotide composition, wherein one or more of the structural elements of the oligonucleotide comprise or consist of one or more structural elements of WV-3511, wherein the composition further comprises a lipid.

In some embodiments, the present disclosure provides a chirally controlled oligonucleotide composition, wherein one or more of the structural elements of the oligonucleotide comprise or consist of one or more structural elements of WV-3512, wherein the composition further comprises a lipid.

In some embodiments, the present disclosure provides a chirally controlled oligonucleotide composition, wherein one or more of the structural elements of the oligonucleotide comprise or consist of one or more structural elements of WV-3513, wherein the composition further comprises a lipid.

In some embodiments, the present disclosure provides a chirally controlled oligonucleotide composition, wherein one or more of the structural elements of the oligonucleotide comprise or consist of one or more structural elements of WV-3514, wherein the composition further comprises a lipid.

In some embodiments, the present disclosure provides a chirally controlled oligonucleotide composition, wherein one or more of the structural elements of the oligonucleotide comprise or consist of one or more structural elements of WV-3515, wherein the composition further comprises a lipid.

In some embodiments, the present disclosure provides chirally controlled oligonucleotide compositions of WV-887, WV-892, WV-896, WV-1714, WV-2444, WV-2445, WV-2526, WV-2527, WV-2528, WV-2530, WV-2531, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546. In some embodiments, the present disclosure provides a chirally controlled oligonucleotide composition of WV-887. In some embodiments, the present disclosure provides a chirally controlled oligonucleotide composition of WV-892. In some embodiments, the present disclosure provides a chirally controlled oligonucleotide composition of WV-896. In some embodiments, the present disclosure provides a chirally controlled oligonucleotide composition of WV-1714. In some embodiments, the present disclosure provides a chirally controlled oligonucleotide composition of WV-2444. In some embodiments, the present disclosure provides a chirally controlled oligonucleotide composition of WV-2445. In some embodiments, the present disclosure provides a chirally controlled oligonucleotide composition of WV-2526. In some embodiments, the present disclosure provides a chirally controlled oligonucleotide composition of WV-2527. In some embodiments, the present disclosure provides a chirally controlled oligonucleotide composition of WV-2528. In some embodiments, the present disclosure provides a chirally controlled oligonucleotide composition of WV-2530. In some embodiments, the present disclosure provides a chirally controlled oligonucleotide composition of WV-2531. In some embodiments, the present disclosure provides a chirally controlled oligonucleotide composition of WV-2578. In some embodiments, the present disclosure provides a chirally controlled oligonucleotide composition of WV-2580. In some embodiments, the present disclosure provides a chirally controlled oligonucleotide composition of WV-2587. In some embodiments, the present disclosure provides a chirally controlled oligonucleotide composition of WV-3047. In some embodiments, the present disclosure provides a chirally controlled oligonucleotide composition of WV-3152. In some embodiments, the present disclosure provides a chirally controlled oligonucleotide composition of WV-3472. In some embodiments, the present disclosure provides a chirally controlled oligonucleotide composition of WV-3473. In some embodiments, the present disclosure provides a chirally controlled oligonucleotide composition of WV-3507. In some embodiments, the present disclosure provides a chirally controlled oligonucleotide composition of WV-3508. In some embodiments, the present disclosure provides a chirally controlled oligonucleotide composition of WV-3509. In some embodiments, the present disclosure provides a chirally controlled oligonucleotide composition of WV-3510. In some embodiments, the present disclosure provides a chirally controlled oligonucleotide composition of WV-3511. In some embodiments, the present disclosure provides a chirally controlled oligonucleotide composition of WV-3512. In some embodiments, the present disclosure provides a chirally controlled oligonucleotide composition of WV-3513. In some embodiments, the present disclosure provides a chirally controlled oligonucleotide composition of WV-3514. In some embodiments, the present disclosure provides a chirally controlled oligonucleotide composition of WV-3515. In some embodiments, the present disclosure provides a chirally controlled oligonucleotide composition of WV-3545. In some embodiments, the present disclosure provides a chirally controlled oligonucleotide composition of WV-3546. As readily appreciated by one skilled in the art, such chirally controlled oligonucleotide compositions comprise predetermined levels of WV-887, WV-892, WV-896, WV-1714, WV-2444, WV-2445, WV-2526, WV-2527, WV-2528, WV-2530, WV-2531, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546.

In some embodiments, a lipid is a fatty acid. In some embodiments, an oligonucleotide is conjugated to a fatty acid. In some embodiments, a fatty acid comprises 10, 11, 12, 13, 14, 15, 16, 17, 18, 19, 20, 21, 22, 23, 24, 25, 26, 27, 28, 29, 30 or more carbon atoms. In some embodiments, a fatty acid comprises 10 or more carbon atoms. In some embodiments, a fatty acid comprises 11 or more carbon atoms. In some embodiments, a fatty acid comprises 12 or more carbon atoms. In some embodiments, a fatty acid comprises 13 or more carbon atoms. In some embodiments, a fatty acid comprises 14 or more carbon atoms. In some embodiments, a fatty acid comprises 15 or more carbon atoms. In some embodiments, a fatty acid comprises 16 or more carbon atoms. In some embodiments, a fatty acid comprises 17 or more carbon atoms. In some embodiments, a fatty acid comprises 18 or more carbon atoms. In some embodiments, a fatty acid comprises 19 or more carbon atoms. In some embodiments, a fatty acid comprises 20 or more carbon atoms. In some embodiments, a fatty acid comprises 21 or more carbon atoms. In some embodiments, a fatty acid comprises 22 or more carbon atoms. In some embodiments, a fatty acid comprises 23 or more carbon atoms. In some embodiments, a fatty acid comprises 24 or more carbon atoms. In some embodiments, a fatty acid comprises 25 or more carbon atoms. In some embodiments, a fatty acid comprises 26 or more carbon atoms. In some embodiments, a fatty acid comprises 27 or more carbon atoms. In some embodiments, a fatty acid comprises 28 or more carbon atoms. In some embodiments, a fatty acid comprises 29 or more carbon atoms. In some embodiments, a fatty acid comprises 30 or more carbon atoms.

In some embodiments, a lipid is stearic acid or turbinaric acid. In some embodiments, a lipid is stearic acid. In some embodiments, a lipid is turbinaric acid.

In some embodiments, a provided oligonucleotide is no more than 25 bases long. In some embodiments, a provided oligonucleotide is no more than 30 bases long. In some embodiments, a provided oligonucleotide is no more than 35 bases long. In some embodiments, a provided oligonucleotide is no more than 40 bases long. In some embodiments, a provided oligonucleotide is no more than 45 bases long. In some embodiments, a provided oligonucleotide is no more than 50 bases long. In some embodiments, a provided oligonucleotide is no more than 55 bases long. In some embodiments, the oligonucleotide is no more than 60 bases long.

In some embodiments, an oligonucleotide comprises one or more chiral internucleotidic linkages. In some embodiments, for oligonucleotides comprising one or more chiral internucleotidic linkages, a provided composition is a stereorandom composition of such oligonucleotides in that stereochemistry of each of the chiral internucleotidic linkages is not controlled. In some embodiments, a stereorandom composition is prepared by oligonucleotide synthesis without dedicated efforts e.g., through chiral auxiliaries, etc. to control the stereochemistry of each chiral internucleotidic linkages. In some embodiments, for oligonucleotides comprising one or more chiral internucleotidic linkages, a provided composition is a chirally controlled oligonucleotide composition of such oligonucleotides in that stereochemistry of at least one of the chiral internucleotidic linkages is controlled. In some embodiments, stereochemistry of each of the chiral internucleotidic linkages is independently controlled, and a provided composition is a completely chirally controlled oligonucleotide composition. In some embodiments, stereochemistry of one or more chiral internucleotidic linkages is controlled (chiral controlled internucleotidic linkages) while stereochemistry of one or more chiral internucleotidic linkages is not controlled (stereorandom/non-chirally controlled internucleotidic linkages), and a provided composition is a partially chirally controlled oligonucleotide composition. In some embodiments, a chirally controlled oligonucleotide composition can be prepared by oligonucleotide synthesis comprising stereoselective formation of one or more or all chiral internucleotidic linkages using, for example, technologies described in Patent Application Publications US20120316224, US20140194610, US20150211006, and WO2015107425, the technologies of each of which are incorporated herein by reference. In some embodiments, a provided composition comprises a chirally controlled oligonucleotide composition described in Patent Application Publications US20120316224, US20140194610, US20150211006, and WO2015107425, the chirally controlled oligonucleotide compositions of each of which are incorporated herein by reference, and a lipid. In some embodiments, a lipid is conjugated to oligonucleotides comprising stereochemically controlled internucleotidic linkages.

In some embodiments, the present disclosure provides an oligonucleotide composition comprising a lipid, and a first plurality of oligonucleotides which have a common base sequence, and comprise one or more modified sugar moieties, one or more natural phosphate linkages, or combinations thereof. In some embodiments, the present disclosure provides a lipid, and an oligonucleotide composition comprising a first plurality of oligonucleotides which have a common base sequence, comprise one or more modified internucleotidic linkages, and comprise one or more modified sugar moieties, one or more natural phosphate linkages, or combinations thereof. In some embodiments, oligonucleotides of a first plurality have a wing-core-wing structure. In some embodiments, each wing region independently comprises one or more natural phosphate linkages and optionally one or more modified internucleotidic linkages, and the core comprises one or more modified internucleotidic linkages and optionally one or more natural phosphate linkages. In some embodiments, each wing region independently comprises one or more natural phosphate linkages and one or more modified internucleotidic linkages, and the core comprises one or more modified internucleotidic linkages and no natural phosphate linkages. In some embodiments, a wing comprises modified sugar moieties. In some embodiments, a modified internucleotidic linkage is phosphorothioate. In some embodiments, a modified internucleotidic linkage is substituted phosphorothioate. In some embodiments, a modified internucleotidic linkage has the structure of formula I described in this disclosure. In some embodiments, a modified sugar moiety is 2′-modified. In some embodiments, a 2′-modification is 2′-OR¹. In some embodiments, a 2′-modification is 2′-R¹.

In some embodiments, a wing comprises at least 3 2′-F modifications. In some embodiments, a wing comprises at least 4 2′-F modifications. In some embodiments, a wing comprises at least 5 2′-F modifications. In some embodiments, a wing comprises at least 6 2′-F modifications. In some embodiments, a core comprising any two or more of: a 2′-F modification, a 2′-OMe modification, or 2′-OH. In some embodiments, a core comprises at least 1 2′-OMe modification. In some embodiments, a core comprises at least 2 2′-OMe modifications. In some embodiments, a core comprises at least 3 2′-OMe modifications. In some embodiments, a core comprises at least 2 2′-OMe modifications. In some embodiments, a core comprises at least 4 2′-OMe modifications. In some embodiments, a core comprises at least 1 2′-F modification. In some embodiments, a core comprises at least 2 2′-F modifications. In some embodiments, a core comprises at least 3 2′-F modifications. In some embodiments, a core comprises at least 2 2′-F modifications. In some embodiments, a core comprises at least 4 2′-F modifications. In some embodiments, a core comprises at least 1 2′-F modification and at least 1 2′-OMe modification. In some embodiments, a core comprises at least 1 2′-F modification and at least 2 2′-OMe modifications. In some embodiments, a core comprises at least 2 2′-F modifications and at least 1 2′-OMe modification. In some embodiments, a core comprises at least 2 2′-F modifications and at least 2 2′-OMe modifications. In some embodiments, the 2′-F modifications in the core and/or wing are contiguous or non-contiguous. In some embodiments, the 2′-OMe modifications in the core and/or wing are contiguous or non-contiguous. In some embodiments, the 2′-OH in the core and/or wing are contiguous or non-contiguous.

In some embodiments, each wing comprises at least one chiral internucleotidic linkage and at least one natural phosphate linkage. In some embodiments, each wing comprises at least one modified sugar moiety. In some embodiments, each wing sugar moiety is modified. In some embodiments, a wing sugar moiety is modified by a modification that is absent from the core region. In some embodiments, a wing region only has modified internucleotidic linkages at one or both of its ends. In some embodiments, a wing region only has a modified internucleotidic linkage at its 5′-end. In some embodiments, a wing region only has a modified internucleotidic linkage at its 3′-end. In some embodiments, a wing region only has modified internucleotidic linkages at its 5′- and 3′-ends. In some embodiments, a wing is to the 5′-end of a core, and the wing only has a modified internucleotidic linkage at its 5′-end. In some embodiments, a wing is to the 5′-end of a core, and the wing only has a modified internucleotidic linkage at its 3′-end. In some embodiments, a wing is to the 5′-end of a core, and the wing only has modified internucleotidic linkages at both its 5′- and 3′-ends. In some embodiments, a wing is to the 3′-end of a core, and the wing only has a modified internucleotidic linkage at its 5′-end. In some embodiments, a wing is to the 3′-end of a core, and the wing only has a modified internucleotidic linkage at its 3′-end. In some embodiments, a wing is to the 3′-end of a core, and the wing only has modified internucleotidic linkages at both its 5′- and 3′-ends.

In some embodiments, a wing comprises at least 4 phosphorothioates. In some embodiments, a wing comprises at least 5 phosphorothioates. In some embodiments, a wing comprises at least 6 phosphorothioates. In some embodiments, a core comprises at least 2 phosphorothioates. In some embodiments, a core comprises at least 3 phosphorothioates. In some embodiments, a core comprises at least 4 phosphorothioates. In some embodiments, a core comprises at least 5 phosphorothioates. In some embodiments, a core comprises at least 6 phosphorothioates. In some embodiments, a core comprises at least 2 phosphodiesters. In some embodiments, a core comprises at least 3 phosphodiesters. In some embodiments, a core comprises at least 4 phosphodiesters. In some embodiments, a core comprises at least 5 phosphodiesters. In some embodiments, a core comprises at least 6 phosphodiesters. In some embodiments, a core comprises at least 1 phosphodiester and at least 1 phosphorothioate. In some embodiments, a core comprises at least 1 phosphodiesters and at least 2 phosphorothioates. In some embodiments, a core comprises at least 2 phosphodiesters and at least 1 phosphorothioates. In some embodiments, a core comprises at least 2 phosphodiesters and at least 2 phosphorothioates. In some embodiments, a core comprises at least 2 phosphodiesters and at least 3 phosphorothioates. In some embodiments, a core comprises at least 3 phosphodiesters and at least 2 phosphorothioates. In some embodiments, a core comprises at least 3 phosphodiesters and at least 3 phosphorothioates. In some embodiments, the phosphodiesters in the core and/or one or both wings are optionally contiguous or not contiguous. In some embodiments, such provided compositions have lower toxicity. In some embodiments, provided compositions have lower complement activation.

In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, a nucleic acid or oligonucleotide or other biologically active agent is capable of reducing the level and/or activity of a mutant form of any of: dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, a nucleic acid or oligonucleotide or other biologically active agent is capable of increasing the level and/or activity of a wild-type and/or functional form of any of: dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).

In some embodiments, provided compositions is a chirally controlled oligonucleotide composition comprising a lipid, which is optionally conjugated with oligonucleotides. In some embodiments, a provided oligonucleotide composition comprising a first plurality of oligonucleotides is chirally controlled, and oligonucleotides of the first plurality comprise a combination of 2′-modification of one or more sugar moieties, one or more natural phosphate linkages, and one or more chiral internucleotidic linkages. In some embodiments, a provided oligonucleotide composition comprising a first plurality of oligonucleotides is chirally controlled, and oligonucleotides of the first plurality comprise a combination of 2′-modification of one or more sugar moieties, one or more natural phosphate linkages, one or more chiral internucleotidic linkages, wherein the 5′- and/or the 3′-end internucleotidic linkages are chiral. In some embodiments, both the 5′- and the 3′-end internucleotidic linkages are chiral. In some embodiments, both the 5′- and the 3′-end internucleotidic linkages are chiral and Sp. In some embodiments, a provided oligonucleotide composition comprising a first plurality of oligonucleotides is chirally controlled, and oligonucleotides of the first plurality comprise a combination of 2′-modification of one or more sugar moieties, one or more natural phosphate linkages, one or more chiral internucleotidic linkages, and a stereochemistry pattern of (Rp)n(Sp)m, (Np)t(Rp)n(Sp)m, or (Sp)t(Rp)n(Sp)m, wherein m>2. In some embodiments, a chiral internucleotidic linkage has the structure of formula I. In some embodiments, a chiral internucleotidic linkage is a phosphorothioate linkage. In some embodiments, a chiral internucleotidic linkage is a substituted phosphorothioate linkage. In some embodiments, oligonucleotides of the first plurality are optionally and independently conjugated to a lipid.

In some embodiments, provided oligonucleotides in provided technologies comprise a wing region and a core region. In some embodiments, provided oligonucleotides have a wing-core-wing structure, wherein the core region comprises one or more sugar moieties and/or internucleotidic linkages not in the wing regions. In some embodiments, provided oligonucleotides have a wing-core-wing structure, wherein the core region comprises one or more sugar moieties and internucleotidic linkages not in the wing regions. In some embodiments, provided oligonucleotides have a wing-core-wing structure, wherein the core region comprises one or more sugar moieties not in the wing regions. In some embodiments, provided oligonucleotides have a wing-core-wing structure, wherein the core region comprises one or more internucleotidic linkages not in the wing regions. In some embodiments, a core region comprises a modified sugar moiety. In some embodiments, each sugar moiety in a core region is modified. Example sugar modifications are widely known in the art including but not limited to those described in this disclosure. In some embodiments, each wing region comprises no modified sugar moieties. In some embodiments, a core region comprises one or more natural phosphate linkages. In some embodiments, each internucleotidic linkage following a core nucleoside is natural phosphate linkage. In some embodiments, a wing comprises one or more modified internucleotidic linkages. In some embodiments, each internucleotidic linkage following a core nucleoside is a modified internucleotidic linkage.

In some embodiments, provided oligonucleotides are blockmers. In some embodiments, provided oligonucleotide are altmers. In some embodiments, provided oligonucleotides are altmers comprising alternating blocks. In some embodiments, a blockmer or an altmer can be defined by chemical modifications (including presence or absence), e.g., base modifications, sugar modification, internucleotidic linkage modifications, stereochemistry, etc.

In some embodiments, provided oligonucleotides comprise blocks comprising different internucleotidic linkages. In some embodiments, provided oligonucleotides comprise blocks comprising modified internucleotidic linkages and natural phosphate linkages. In some embodiments, provided oligonucleotides comprise blocks comprising different modified internucleotidic linkages. In some embodiments, provided oligonucleotides comprise alternating blocks comprising different internucleotidic linkages. In some embodiments, provided oligonucleotides comprise alternating blocks comprising modified internucleotidic linkages and natural phosphate linkages. In some embodiments, provided oligonucleotides comprise alternating blocks comprising different modified internucleotidic linkages. In some embodiments, a block comprising modified internucleotidic linkages have pattern of backbone chiral centers as described herein. In some embodiments, each block comprising modified internucleotidic linkages has the same pattern of backbone chiral centers. In some embodiments, blocks comprising modified internucleotidic linkages have different patterns of backbone chiral centers. In some embodiments, blocks comprising modified internucleotidic linkages have different length and/or modifications. In some embodiments, blocks comprising modified internucleotidic linkages have the same length and/or modifications. In some embodiments, blocks comprising modified internucleotidic linkages have the same length. In some embodiments, blocks comprising modified internucleotidic linkages have the same internucleotidic linkages. In some embodiments, provided oligonucleotides comprise a first block at the 5′-end (5′-block), and a second block at the 3′-end (3′-block), each of which independently comprise one or more modified internucleotidic linkages. In some embodiments, each of the 5′- and 3′-blocks independently comprises 2, 3, 4, 5, 6, 7, 8, 9, 10, 11, 12, 13, 14, 15, 16, 17, 18, 19, 20 or more modified internucleotidic linkages. In some embodiments, a 5′-block comprises 4 or more modified internucleotidic linkages. In some embodiments, a 5′-block comprises 5 or more modified internucleotidic linkages. In some embodiments, a 5′-block comprises 6 or more modified internucleotidic linkages. In some embodiments, a 5′-block comprises 7 or more modified internucleotidic linkages. In some embodiments, a 3′-block comprises 4 or more modified internucleotidic linkages. In some embodiments, a 3′-block comprises 5 or more modified internucleotidic linkages. In some embodiments, a 3′-block comprises 6 or more modified internucleotidic linkages. In some embodiments, a 3′-block comprises 7 or more modified internucleotidic linkages. In some embodiments, each of the 5′- and 3′-blocks independently comprises at least 4 modified internucleotidic linkages. In some embodiments, each of the 5′- and 3′-blocks independently comprises at least 5 modified internucleotidic linkages. In some embodiments, each of the 5′- and 3′-blocks independently comprises at least 6 modified internucleotidic linkages. In some embodiments, each of the 5′- and 3′-blocks independently comprises at least 7 modified internucleotidic linkages. In some embodiments, modified internucleotidic linkages within a block are consecutive. In some embodiments, each linkage of the 5′-block is independently a modified internucleotidic linkage. In some embodiments, each linkage of the 5′-block is independently a phosphorothioate linkage. In some embodiments, each linkage of the 5′-block is independently chirally controlled. In some embodiments, each linkage of the 5′-block is Sp. In some embodiments, each linkage of the 3′-block is independently a modified internucleotidic linkage. In some embodiments, each linkage of the 3′-block is independently a phosphorothioate linkage. In some embodiments, each linkage of the 3′-block is independently chirally controlled. In some embodiments, each linkage of the 3′-block is Sp.

In some embodiments, provided oligonucleotides comprise blocks comprising sugar modifications. In some embodiments, provided oligonucleotides comprise one or more blocks comprising one or more 2′-F modifications (2′-F blocks). In some embodiments, provided oligonucleotides comprise blocks comprising consecutive 2′-F modifications. In some embodiments, a block comprises 2, 3, 4, 5, 6, 7, 8, 9, 10, 11, 12, 13, 14, 15, 16, 17, 18, 19, 20 or more consecutive 2′-F modifications. In some embodiments, a block comprises 4 or more 2′-F modifications. In some embodiments, a block comprises 5 or more 2′-F modifications. In some embodiments, a block comprises 6 or more 2′-F modifications. In some embodiments, a block comprises 7 or more 2′-F modifications. In some embodiments, provided oligonucleotides comprises one or more blocks comprising one or more 2′-OR¹ modifications (2′-OR¹ blocks). In some embodiments, provided oligonucleotides comprise both 2′-F and 2′-OR¹ blocks. In some embodiments, provided oligonucleotides comprise alternating 2′-F and 2′-OR¹ blocks. In some embodiments, provided oligonucleotides comprise a first 2′-F block at the 5′-end, and a second 2′-F block at the 3′-end, each of which independently comprises 2, 3, 4, 5, 6, 7, 8, 9, 10, 11, 12, 13, 14, 15, 16, 17, 18, 19, 20 or more consecutive 2′-F modifications; in some embodiments, each of which independently comprises 4 or more 2′-F modifications; in some embodiments, each of which independently comprises 5 or more 2′-F modifications; in some embodiments, each of which independently comprises 6 or more 2′-F modifications; in some embodiments, each of which independently comprises 7 or more 2′-F modifications. In some embodiments, provided oligonucleotides comprise a 5′-block wherein each sugar moiety of the 5′-block comprises a 2′-F modification. In some embodiments, provided oligonucleotides comprise a 3′-block wherein each sugar moiety of the 3′-block comprises a 2′-F modification. In some embodiments, such provided oligonucleotides comprise one or more 2′-OR¹ blocks, and optionally one or more 2′-F blocks, between the 5′ and 3′ 2′-F blocks. In some embodiments, such provided oligonucleotides comprise one or more 2′-OR¹ blocks, and one or more 2′-F blocks, between the 5′ and 3′ 2′-F blocks (e.g., WV-3407, WV-3408, etc.).

In some embodiments, provided oligonucleotides comprise one or more 2′-F modified sugar moieties whose 3′-internucleotidic linkages are modified internucleotidic linkages. In some embodiments, a modified internucleotidic linkage is phosphorothioate. In some embodiments, a modified internucleotidic linkage is chirally controlled and is Rp. In some embodiments, a modified internucleotidic linkage is chirally controlled and is Sp. In some embodiments, provided oligonucleotides comprise one or more 2′-OR¹ modified sugar moieties whose 3′-internucleotidic linkages are natural phosphate linkages.

In some embodiments, a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp. In some embodiments, A and G are followed by natural phosphate linkage (PO).

In some embodiments, provided oligonucleotides comprise alternating blocks comprising modified sugar moieties and unmodified sugar moieties. In some embodiments, modified sugar moieties comprise 2′-modifications. In some embodiments, provided oligonucleotides comprise alternating 2′-OMe modified sugar moieties and unmodified sugar moieties. For examples, see WV-1112, WV-1113, etc.

In some embodiments, provided oligonucleotides comprise alternating blocks comprising different modified sugar moieties and/or unmodified sugar moieties. In some embodiments, provided oligonucleotides comprise alternating blocks comprising different modified sugar moieties and unmodified sugar moieties. In some embodiments, provided oligonucleotides comprise alternating blocks comprising different modified sugar moieties. In some embodiments, provided oligonucleotides comprise alternating blocks comprising different modified sugar moieties, wherein the modified sugar moieties comprise different 2′-modifications. For example, in some embodiments, provided oligonucleotide comprises alternating blocks comprising 2′-OMe and 2′-F, respectively. For examples, see WV-1712, WV1713, WV-1714, etc.

In some embodiments, a type of nucleoside in a region or an oligonucleotide is modified, optionally with a different modification compared to another type of nucleoside. In some embodiments, a type of nucleoside in a region or an oligonucleotide is modified with a different modification compared to another type of nucleoside. For example, in some embodiments, a pyrimidine nucleoside comprises a 2′-F modification, and a purine nucleoside comprises a 2′-OMe modification. In some other embodiments, a pyrimidine nucleoside comprises a 2′-OMe modification, and a purine nucleoside comprises a 2′-F modification. In some embodiments, G and C has one type of sugar modification, and A and U has another type of sugar modification. In some embodiments, G and C comprises 2′-OMe modification, and A and U comprises 2′-F modification. In some embodiments, G and C comprises 2′-F modification, and A and U comprises 2′-OMe modification.

In some embodiments, an internucleotidic linkage following an unmodified sugar moiety is a modified internucleotidic linkage. In some embodiments, an internucleotidic linkage after an unmodified sugar moiety is a phosphorothioate linkage. In some embodiments, each internucleotidic linkage after an unmodified sugar moiety is a modified internucleotidic linkage. In some embodiments, each internucleotidic linkage after an unmodified sugar moiety is a phosphorothioate linkage. In some embodiments, an internucleotidic linkage following a modified sugar moiety is a natural phosphate linkage. In some embodiments, each internucleotidic linkage following a modified sugar moiety is a natural phosphate linkage.

In some embodiments, a provided pattern of backbone chiral centers comprises repeating (Sp)m(Rp)n, (Rp)n(Sp)m, (Np)t(Rp)n(Sp)m, or (Sp)t(Rp)n(Sp)m units. In some embodiments, a repeating unit is (Sp)m(Rp)n. In some embodiments, a repeating unit is SpRp. In some embodiments, a repeating unit is SpSpRp. In some embodiments, a repeating unit is SpRpRp. In some embodiments, a repeating unit is RpRpSp. In some embodiments, a repeating unit is (Rp)n(Sp)m. In some embodiments, a repeating unit is (Np)t(Rp)n(Sp)m. In some embodiments, a repeating unit is (Sp)t(Rp)n(Sp)m.

In some embodiments, a provided pattern of backbone chiral centers comprises a (Sp)m(Rp)n, (Rp)n(Sp)m, (Np)t(Rp)n(Sp)m, or (Sp)t(Rp)n(Sp)m unit. In some embodiments, a unit is (Sp)m(Rp)n. In some embodiments, a unit is SpRp. In some embodiments, a unit is SpSpRp. In some embodiments, a unit is SpRpRp. In some embodiments, a unit is RpRpSp. In some embodiments, a unit is (Rp)n(Sp)m. In some embodiments, a unit is (Sp)m(Rp)n. In some embodiments, a unit is (Rp)n(Sp)m. In some embodiments, a unit is (Np)t(Rp)n(Sp)m. In some embodiments, a unit is (Sp)t(Rp)n(Sp)m.

In some embodiments, a provided pattern of backbone chiral centers comprises (Rp/Sp)-(All Rp or All Sp)-(Rp/Sp). In some embodiments, a provided pattern of backbone chiral centers comprises (Rp)-(All Sp)-(Rp). In some embodiments, a provided pattern of backbone chiral centers comprises (Sp)-(All Rp)-(Sp). In some embodiments, a provided pattern of backbone chiral centers comprises (Rp/Sp)-(repeating (Sp)m(Rp)n)-(Rp/Sp). In some embodiments, a provided pattern of backbone chiral centers comprises (Rp/Sp)-(repeating SpSpRp)-(Rp/Sp).

In some embodiments, a provided pattern of backbone chiral centers is (Rp/Sp)-(All Rp or All Sp)-(Rp/Sp). In some embodiments, a provided pattern of backbone chiral centers is (Sp)-(All Sp)-(Sp). In some embodiments, each chiral internucleotidic linkage is Sp. In some embodiments, a provided pattern of backbone chiral centers is (Rp)-(All Sp)-(Rp). In some embodiments, a provided pattern of backbone chiral centers is (Sp)-(All Rp)-(Sp). In some embodiments, a provided pattern of backbone chiral centers is (Rp/Sp)-(repeating (Sp)m(Rp)n)-(Rp/Sp). In some embodiments, a provided pattern of backbone chiral centers is (Rp/Sp)-(repeating SpSpRp)-(Rp/Sp).

In some embodiments, the present disclosure provides oligonucleotide compositions having low toxicity. In some embodiments, the present disclosure provides oligonucleotide compositions having improved protein binding profile. In some embodiments, the present disclosure provides oligonucleotide compositions having improved binding to albumin. In some embodiments, provided compositions have low toxicity and improved binding to certain desired proteins. In some embodiments, provided compositions have low toxicity and improved binding to certain desired proteins. In some embodiments, provided oligonucleotide compositions at the same time provides the same level of, or greatly enhanced, stability and/or activities, e.g., better target-cleavage pattern, better target-cleavage efficiency, better target specificity, etc.

In some embodiments, the present disclosure provides an oligonucleotide composition comprising a lipid and a first plurality of oligonucleotides which:
1) have a common base sequence complementary to a target sequence in a transcript; and
2) comprise one or more modified sugar moieties and modified internucleotidic linkages; wherein the lipid is optionally conjugated to one or more oligonucleotides of the plurality.

In some embodiments, a provided oligonucleotide composition is characterized in that, when it is contacted with the transcript in a transcript splicing system, splicing of the transcript is altered relative to that observed under reference conditions selected from the group consisting of absence of a lipid of the composition, absence of the composition, presence of a reference composition, and combinations thereof.

In some embodiments, a reference condition is absence of lipids in the composition. In some embodiments, a reference condition is absence of the composition. In some embodiments, a reference condition is presence of a reference composition. Example reference compositions comprising a reference plurality of oligonucleotides are extensively described in this disclosure. In some embodiments, oligonucleotides of the reference plurality have a different structural elements (chemical modifications, stereochemistry, etc.) compared with oligonucleotides of the first plurality in a provided composition. In some embodiments, a reference composition is a stereorandom preparation of oligonucleotides having the same chemical modifications. In some embodiments, a reference composition is a mixture of stereoisomers while a provided composition is a chirally controlled oligonucleotide composition of one stereoisomer. In some embodiments, oligonucleotides of the reference plurality have the same base sequence as oligonucleotide of the first plurality in a provided composition. In some embodiments, oligonucleotides of the reference plurality have the same chemical modifications as oligonucleotide of the first plurality in a provided composition. In some embodiments, oligonucleotides of the reference plurality have the same sugar modifications as oligonucleotide of the first plurality in a provided composition. In some embodiments, oligonucleotides of the reference plurality have the same base modifications as oligonucleotide of the first plurality in a provided composition. In some embodiments, oligonucleotides of the reference plurality have the same internucleotidic linkage modifications as oligonucleotide of the first plurality in a provided composition. In some embodiments, oligonucleotides of the reference plurality have the same stereochemistry as oligonucleotide of the first plurality in a provided composition but different chemical modifications, e.g., base modification, sugar modification, internucleotidic linkage modifications, etc. In some embodiments, oligonucleotides of the reference plurality differ only in that they are not conjugated to lipids.

In some embodiments, provided oligonucleotide compositions have lower toxicity. In some embodiments, provided oligonucleotide oligonucleotides have improved safety profile. In some embodiments, provided oligonucleotide compositions provided better protein binding properties.

Example splicing systems are widely known in the art. In some embodiments, a splicing system is an in vivo or in vitro system including components sufficient to achieve splicing of a relevant target transcript. In some embodiments, a splicing system is or comprises a spliceosome (e.g., protein and/or RNA components thereof). In some embodiments, a splicing system is or comprises an organellar membrane (e.g., a nuclear membrane) and/or an organelle (e.g., a nucleus). In some embodiments, a splicing system is or comprises a cell or population thereof. In some embodiments, a splicing system is or comprises a tissue. In some embodiments, a splicing system is or comprises an organism, e.g., an animal, e.g., a mammal such as a mouse, rat, monkey, human, etc.

In some embodiments, conjugation of oligonucleotides with lipids may improve oligonucleotide properties, e.g., activities, toxicities, etc. In some embodiments, as demonstrated by the present disclosure, conjugation may improve activities of oligonucleotides. In some embodiments, as demonstrated by the present disclosure, conjugation may improve stability of oligonucleotides. In some embodiments, as demonstrated by the present disclosure, conjugation may improve delivery of oligonucleotides to target locations. In some embodiments, as demonstrated by the present disclosure, conjugation may improve delivery of oligonucleotides into cells. In some embodiments, as demonstrated by the present disclosure, conjugation may improve delivery of oligonucleotides into cells in a subject. In some embodiments, as demonstrated by the present disclosure, conjugation may improve activity, safety, stability, and/or delivery of oligonucleotides.

In some embodiments, the present disclosure provides an oligonucleotide composition comprising one or more lipids, and a first plurality of oligonucleotides which:
1) have a common base sequence complementary to a target sequence in a transcript; and
2) comprise one or more modified sugar moieties and modified internucleotidic linkages, the oligonucleotide composition being characterized in that, when it is contacted with the transcript in a transcript splicing system, splicing of the transcript is altered relative to that observed under reference conditions selected from the group consisting of absence of the composition, presence of a reference composition, and combinations thereof, wherein the lipids are optionally and independently conjugated to one or more oligonucleotides of the plurality.

In some embodiments, the present disclosure provides an oligonucleotide composition comprising one or more lipids, and a first plurality of oligonucleotides of a particular oligonucleotide type defined by:
1) base sequence;
2) pattern of backbone linkages;
3) pattern of backbone chiral centers; and
4) pattern of backbone phosphorus modifications;

wherein the lipids are optionally and independently conjugated to one or more oligonucleotides of the plurality, and level of oligonucleotides of the plurality is predetermined.

In some embodiments, the present disclosure provides an oligonucleotide composition comprising one or more lipids and a first plurality of oligonucleotides of a particular oligonucleotide type defined by:
1) base sequence;
2) pattern of backbone linkages;
3) pattern of backbone chiral centers; and
4) pattern of backbone phosphorus modifications,
   which composition is chirally controlled in that it is enriched, relative to a substantially racemic preparation of oligonucleotides having the same base sequence, for oligonucleotides of the particular oligonucleotide type,

the oligonucleotide composition being characterized in that, when it is contacted with the transcript in a transcript splicing system, splicing of the transcript is altered relative to that observed under reference conditions selected from the group consisting of absence of the composition, presence of a reference composition, and combinations thereof, and

wherein the lipids are optionally and independently conjugated to one or more oligonucleotides of the plurality, and level of oligonucleotides of the plurality is predetermined.

In some embodiments, the present disclosure provides an oligonucleotide composition comprising one or more lipids, and a first plurality of oligonucleotides comprising one or more wing regions and a core region, wherein:

oligonucleotides of the first plurality have the same base sequence; and

each wing region independently comprises one or more modified internucleotidic linkages and optionally one or more natural phosphate linkages, and the core region independently comprises one or more modified internucleotidic linkages; or

each wing region independently comprises one or more modified sugar moieties, and the core region comprises one or more un-modified sugar moieties; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising one or more wing regions and a core region, wherein:

oligonucleotides of the first plurality have the same base sequence;

each wing region independently has a length of two or more bases, and independently comprises one or more modified internucleotidic linkages and optionally one or more natural phosphate linkages; and

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one or more lipids and a first plurality of oligonucleotides comprising one or more wing regions and a core region, wherein:

oligonucleotides of the first plurality have the same base sequence;

each wing region independently has a length of two or more bases, and independently comprises one or more modified internucleotidic linkages and one or more natural phosphate linkages; and

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one ore more lipids, and a first plurality of oligonucleotides comprising two wing regions and a core region, wherein:

oligonucleotides of the first plurality have the same base sequence;

each wing region independently has a length of two or more bases, and independently comprises one or more modified internucleotidic linkages and one or more natural phosphate linkages; and

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising two wing regions and a core region, wherein:

oligonucleotides of the first plurality have the same base sequence;

each wing region independently has a length of two or more bases, and independently comprises one or more modified internucleotidic linkages and one or more natural phosphate linkages;

the wing region to the 5′-end of the core region comprises at least one modified internucleotidic linkage followed by a natural phosphate linkage in the wing; and

the wing region to the 3′-end of the core region comprises at least one modified internucleotidic linkage preceded by a natural phosphate linkage in the wing;

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising a wing region and a core region, wherein:

oligonucleotides of the first plurality have the same base sequence;

the wing region has a length of two or more bases, and comprises one or more modified internucleotidic linkages and one or more natural phosphate linkages;

the wing region is to the 5′-end of the core region and comprises a natural phosphate linkage between the two nucleosides at its 3′-end, or the wing region to the 3′-end of the core region and comprises a natural phosphate linkage between the two nucleosides at its 5′-end; and

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising two wing regions and a core region, wherein:

oligonucleotides of the first plurality have the same base sequence;

each wing region independently has a length of two or more bases, and independently comprises one or more modified internucleotidic linkages and one or more natural phosphate linkages;

the wing region to the 5′-end of the core region comprises a natural phosphate linkage between the two nucleosides at its 3′-end;

the wing region to the 3′-end of a core region comprises a natural phosphate linkage between the two nucleosides at its 5′-end; and

the core region independently has a length of two or more bases and independently comprises one or more modified internucleotidic linkages; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising one or more wing regions and a core region, wherein:

oligonucleotides of the first plurality have the same base sequence; and

each wing region independently comprises one or more modified internucleotidic linkages and optionally one or more natural phosphate linkages, and the core region independently comprises one or more modified internucleotidic linkages; and

each wing region independently comprises one or more modified sugar moieties, and the core region comprises one or more un-modified sugar moieties; and

wherein the lipids are optionally and independently conjugated to one or more oligonucleotides of the plurality.

In some embodiments, the present disclosure provides an oligonucleotide composition comprising one or more lipids, and a first plurality of oligonucleotides comprising one or more wing regions and a core region, wherein:

oligonucleotides of the first plurality have the same base sequence; and

each wing region independently comprises one or more modified internucleotidic linkages and one or more natural phosphate linkages, and the core region independently comprises one or more modified internucleotidic linkages; and

each wing region independently comprises one or more modified sugar moieties, and the core region comprises one or more un-modified sugar moieties; and

wherein the lipids are optionally and independently conjugated to one or more oligonucleotides of the plurality.

In some embodiments, the present disclosure provides an oligonucleotide composition comprising one or more lipids, and a first plurality of oligonucleotides which:
1) have a common base sequence; and
2) comprise one or more wing regions and a core region;
   wherein:

each wing region comprises at least one modified sugar moiety; and

each core region comprises at least one un-modified sugar moiety; and

wherein the lipids are optionally and independently conjugated to one or more oligonucleotides of the plurality.

In some embodiments, the present disclosure provides a chirally controlled oligonucleotide composition comprising one or more lipids, and oligonucleotides defined by having:
1) a common base sequence and length;
2) a common pattern of backbone linkages; and
3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers; and

wherein the lipids are optionally conjugated to one or more of the defined oligonucleotides.

In some embodiments, the present disclosure provides a chirally controlled oligonucleotide composition comprising one or more lipids, and oligonucleotides of a particular oligonucleotide type characterized by:
1) a common base sequence and length;
2) a common pattern of backbone linkages; and
3) a common pattern of backbone chiral centers;
   which composition is chirally controlled in that it is enriched, relative to a substantially racemic preparation of oligonucleotides having the same base sequence and length, for oligonucleotides of the particular oligonucleotide type; and

wherein the lipids are optionally conjugated to one or more oligonucleotides of the oligonucleotide type.

In some embodiments, the present disclosure provides a chirally controlled oligonucleotide composition comprising one or more lipids, and oligonucleotides of a particular oligonucleotide type characterized by:
1) a common base sequence and length;
2) a common pattern of backbone linkages; and
3) a common pattern of backbone chiral centers, which composition is a substantially pure preparation of a single oligonucleotide in that at least about 10% of the oligonucleotides in the composition have the common base sequence and length, the common pattern of backbone linkages, and the common pattern of backbone chiral centers; and

wherein the lipids are optionally and independently conjugated to one or more oligonucleotides of the oligonucleotide type.

In some embodiments, the present disclosure provides an oligonucleotide composition comprising one or more lipids, and a predetermined level of oligonucleotides which comprise one or more wing regions and a common core region, wherein:
each wing region independently has a length of two or more bases, and independently and optionally comprises one or more chiral internucleotidic linkages;
the core region independently has a length of two or more bases, and independently comprises one or more chiral internucleotidic linkages, and the common core region has:
1) a common base sequence and length;
2) a common pattern of backbone linkages; and
3) a common pattern of backbone chiral centers; and

wherein the lipids are optionally and independently conjugated to one or more of the oligonucleotides.

In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).

In some embodiments, levels of defined oligonucleotides in provided compositions (e.g., oligonucleotides of a plurality; oligonucleotides of an oligonucleotide type, oligonucleotides defined by sequence, backbone linkages, and/or backbone chiral centers, etc.) are predetermined. In some embodiments, levels of defined oligonucleotides are predetermined in that their absolute or relative (e.g., ratio, percentage, etc.) amounts within a composition is controlled.

A wing and core can be defined by any structural elements. In some embodiments, a wing and core is defined by nucleoside modifications, wherein a wing comprises a nucleoside modification that the core region does not have. In some embodiments, oligonucleotides in provided compositions have a wing-core structure of nucleoside modification. In some embodiments, oligonucleotides in provided compositions have a core-wing structure of nucleoside modification. In some embodiments, oligonucleotides in provided compositions have a wing-core-wing structure of nucleoside modification. In some embodiments, a wing and core is defined by modifications of the sugar moieties. In some embodiments, a wing and core is defined by modifications of the base moieties. In some embodiments, each sugar moiety in the wing region has the same 2′-modification which is not found in the core region. In some embodiments, each sugar moiety in the wing region has the same 2′-modification which is different than any sugar modifications in the core region. In some embodiments, each sugar moiety in the wing region has the same 2′-modification, and the core region has no 2′-modifications. In some embodiments, when two or more wings are present, each sugar moiety in a wing region has the same 2′-modification, yet the common 2′-modification in a first wing region can either be the same as or different from the common 2′-modification in a second wing region. In some embodiments, a wing and core is defined by pattern of backbone internucleotidic linkages. In some embodiments, a wing comprises a type of internucleotidic linkage, and/or a pattern of internucleotidic linkages, that are not found in a core. In some embodiments, a wing region comprises both a modified internucleotidic linkage and a natural phosphate linkage. In some embodiments, the internucleotidic linkage at the 5′-end of a wing to the 5′-end of the core region is a modified internucleotidic linkage. In some embodiments, the internucleotidic linkage at the 3′-end of a wing to the 3′-end of the core region is a modified internucleotidic linkage. In some embodiments, a modified internucleotidic linkage is a chiral internucleotidic linkage.

In some embodiments, each wing comprises at least one chiral internucleotidic linkage and at least one natural phosphate linkage. In some embodiments, each wing comprises at least one modified sugar moiety. In some embodiments, each wing sugar moiety is modified. In some embodiments, a wing sugar moiety is modified by a modification that is absent from the core region. In some embodiments, a wing region only has modified internucleotidic linkages at one or both of its ends. In some embodiments, a wing region only has a modified internucleotidic linkage at its 5′-end. In some embodiments, a wing region only has a modified internucleotidic linkage at its 3′-end. In some embodiments, a wing region only has modified internucleotidic linkages at its 5′- and 3′-ends. In some embodiments, a wing is to the 5′-end of a core, and the wing only has a modified internucleotidic linkage at its 5′-end. In some embodiments, a wing is to the 5′-end of a core, and the wing only has a modified internucleotidic linkage at its 3′-end. In some embodiments, a wing is to the 5′-end of a core, and the wing only has modified internucleotidic linkages at both its 5′- and 3′-ends. In some embodiments, a wing is to the 3′-end of a core, and the wing only has a modified internucleotidic linkage at its 5′-end. In some embodiments, a wing is to the 3′-end of a core, and the wing only has a modified internucleotidic linkage at its 3′-end. In some embodiments, a wing is to the 3′-end of a core, and the wing only has modified internucleotidic linkages at both its 5′- and 3′-ends.

In some embodiments, each internucleotidic linkage within a core region is modified. In some embodiments, each internucleotidic linkage within a core region is chiral. In some embodiments, a core region comprises a pattern of backbone chiral centers of (Sp)m(Rp)n, (Rp)n(Sp)m, (Np)t(Rp)n(Sp)m, or (Sp)t(Rp)n(Sp)m. In some embodiments, the pattern of backbone chiral centers of a core region is (Sp)m(Rp)n, (Rp)n(Sp)m, (Np)t(Rp)n(Sp)m, or (Sp)t(Rp)n(Sp)m. In some embodiments, a core region comprises a pattern of backbone chiral centers of (Rp)n(Sp)m, (Np)t(Rp)n(Sp)m, or (Sp)t(Rp)n(Sp)m, wherein m>2. In some embodiments, the pattern of backbone chiral centers of a core region is (Sp)m(Rp)n, (Rp)n(Sp)m, (Np)t(Rp)n(Sp)m, or (Sp)t(Rp)n(Sp)m, wherein m>2. Among other things, in some embodiments such patterns can provide or enhance controlled cleavage of a target sequence, e.g., an RNA sequence.

In some embodiments, oligonucleotides in provided compositions have a common pattern of backbone phosphorus modifications. In some embodiments, a provided composition is an oligonucleotide composition that is chirally controlled in that the composition contains a predetermined level of oligonucleotides of an individual oligonucleotide type, wherein an oligonucleotide type is defined by:
1) base sequence;
2) pattern of backbone linkages;
3) pattern of backbone chiral centers; and
4) pattern of backbone phosphorus modifications.

As noted above and understood in the art, in some embodiments, base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, a particular oligonucleotide type may be defined by
1A) base identity;
1B) pattern of base modification;
1C) pattern of sugar modification;
2) pattern of backbone linkages;
3) pattern of backbone chiral centers; and
4) pattern of backbone phosphorus modifications.

Thus, in some embodiments, oligonucleotides of a particular type may share identical bases but differ in their pattern of base modifications and/or sugar modifications. In some embodiments, oligonucleotides of a particular type may share identical bases and pattern of base modifications (including, e.g., absence of base modification), but differ in pattern of sugar modifications. In some embodiments, oligonucleotides of a particular type are chemically identical in that they have the same base sequence (including length), the same pattern of chemical modifications to sugar and base moieties, the same pattern of backbone linkages (e.g., pattern of natural phosphate linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as —S—, and -L-R¹ of formula I).

In some embodiments, the present disclosure provides chirally controlled oligonucleotide compositions of oligonucleotides comprising multiple (e.g., more than 5, 6, 7, 8, 9, or 10) internucleotidic linkages, and particularly for oligonucleotides comprising multiple (e.g., more than 5, 6, 7, 8, 9, or 10) chiral internucleotidic linkages. In some embodiments, in a stereorandom or racemic preparation of oligonucleotides, at least one chiral internucleotidic linkage is formed with less than 90:10, 95:5, 96:4, 97:3, or 98:2 diastereoselectivity. In some embodiments, for a stereoselective or chirally controlled preparation of oligonucleotides, each chiral internucleotidic linkage is formed with greater than 90:10, 95:5, 96:4, 97:3, or 98:2 diastereoselectivity. In some embodiments, for a stereoselective or chirally controlled preparation of oligonucleotides, each chiral internucleotidic linkage is formed with greater than 95:5 diastereoselectivity. In some embodiments, for a stereoselective or chirally controlled preparation of oligonucleotides, each chiral internucleotidic linkage is formed with greater than 96:4 diastereoselectivity. In some embodiments, for a stereoselective or chirally controlled preparation of oligonucleotides, each chiral internucleotidic linkage is formed with greater than 97:3 diastereoselectivity. In some embodiments, for a stereoselective or chirally controlled preparation of oligonucleotides, each chiral internucleotidic linkage is formed with greater than 98:2 diastereoselectivity. In some embodiments, for a stereoselective or chirally controlled preparation of oligonucleotides, each chiral internucleotidic linkage is formed with greater than 99:1 diastereoselectivity. In some embodiments, diastereoselectivity of a chiral internucleotidic linkage in an oligonucleotide may be measured through a model reaction, e.g. formation of a dimer under essentially the same or comparable conditions wherein the dimer has the same internucleotidic linkage as the chiral internucleotidic linkage, the 5′-nucleoside of the dimer is the same as the nucleoside to the 5′-end of the chiral internucleotidic linkage, and the 3′-nucleoside of the dimer is the same as the nucleoside to the 3′-end of the chiral internucleotidic linkage.

As described herein, provided compositions and methods are capable of altering splicing of transcripts. In some embodiments, provided compositions and methods provide improved splicing patterns of transcripts compared to reference conditions selected from the group consisting of absence of the composition, presence of a reference composition, and combinations thereof. An improvement can be an improvement of any desired biological functions. In some embodiments, for example, in DMD, an improvement is production of an mRNA from which a dystrophin protein with improved biological activities is produced. In some other embodiments, for example, an improvement is down-regulation of STAT3, HNRNPH1 and/or KDR to mitigate tumor progression, malignancy, and angiogenesis through forced splicing-induced nonsense-mediated decay (DSD-NMD).

In some embodiments, the present disclosure provides methods for modulating levels of target nucleic acids in a system comprising administering a provided composition. In some embodiments, a system is an in vitro system. In some embodiments, a system is a cell. In some embodiments, a system is a tissue. In some embodiments, a system is an organ. In some embodiments, a system is a subject. In some embodiments, a target nucleic acid is genomic DNA. In some embodiments, a target nucleic acid is a transcript. In some embodiments, a target nucleic acid is a primary transcript. In some embodiments, a target nucleic acid is a processed transcript. In some embodiments, a target nucleic acid is a spliced transcript. In some embodiments, a target nucleic acid is RNA. In some embodiments, a target nucleic acid is mRNA. In some embodiments, a target nucleic acid is pre-mRNA. In some embodiments, technologies of the present disclosure which comprise one or more lipids provide better delivery to target locations, better safety, better activity, better stability, and/or better overall results. etc. compared to absence of the lipids.

In some embodiments, the present disclosure provides a method for altering splicing of a target transcript, comprising administering a provided composition comprising one or more lipids, wherein the splicing of the target transcript is altered relative to reference conditions selected from the group consisting of absence of the lipids, absence of the composition, presence of a reference composition, and combinations thereof.

In some embodiments, the present disclosure provides a method of generating a set of spliced products from a target transcript, the method comprising steps of:

contacting a splicing system containing the target transcript with a provided oligonucleotide composition comprising one or more lipids and a first plurality of oligonucleotides, in an amount, for a time, and under conditions sufficient for a set of spliced products to be generated that is different from a set generated under reference conditions selected from the group consisting of absence of the lipids, absence of the composition, presence of a reference composition, and combinations thereof.

As widely known in the art, many diseases and/or conditions are associated with transcript splicing. For examples, see Garcia-Blanco, et al., Alternative splicing in disease and therapy, Nat. Biotechnol. 2004 May; 22(5):535-46; Wang, et al., Splicing in disease: disruption of the splicing code and the decoding machinery, Nat. Rev. Genet. 2007 October; 8(10):749-61; Havens, et al., Targeting RNA splicing for disease therapy, Wiley Interdiscip. Rev. RNA. 2013 May-June; 4(3):247-66. In some embodiments, the present disclosure provides compositions and methods for treating or preventing diseases.

In some embodiments, the present disclosure provides a method for treating or preventing a disease, comprising administering to a subject an oligonucleotide composition described herein.

In some embodiments, the present disclosure provides a method for treating or preventing a disease, comprising administering to a subject a provided oligonucleotide composition.

In some embodiments, the present disclosure provides a method for treating or preventing a disease, comprising administering a provided oligonucleotide composition,

the oligonucleotide composition being characterized in that, when it is contacted with the transcript in a transcript splicing system, splicing of the transcript is altered relative to that observed under reference conditions selected from the group consisting of absence of the composition, presence of a reference composition, and combinations thereof.

In some embodiments, a disease is one in which, after administering a provided composition, one or more spliced transcripts repair, restore or introduce a new beneficial function. For example, in DMD, after skipping one or more exons, functions of dystrophin can be restored, or partially restored, through a truncated but (partially) active version. In some embodiments, a disease is one in which, after administering a provided composition, one or more spliced transcripts repair, a gene is effectively knockdown by altering splicing of the gene transcript.

In some embodiments, a disease is Duchenne muscular dystrophy. In some embodiments, a disease is spinal muscular atrophy (SMA). In some embodiments, a disease is cancer.

In general, properties of oligonucleotide compositions as described herein can be assessed using any appropriate assay. Relative toxicity and/or protein binding properties and/or activity and/or delivery for different compositions (e.g., stereocontrolled vs non-stereocontrolled, and/or different stereocontrolled compositions) are typically desirably determined in the same assay, in some embodiments substantially simultaneously and in some embodiments with reference to historical results.

Those of skill in the art will be aware of and/or will readily be able to develop appropriate assays for particular oligonucleotide compositions. The present disclosure provides descriptions of certain particular assays, for example that may be useful in assessing one or more features of oligonucleotide composition behavior e.g., complement activation, injection site inflammation, protein biding, etc.

For example, certain assays that may be useful in the assessment of toxicity and/or protein binding properties and/or activity and/or delivery of oligonucleotide compositions may include any assay described and/or exemplified herein.

In some embodiments, the present disclosure demonstrates that oligonucleotide compositions comprising oligonucleotides with conjugation to one or more lipids and controlled structural elements, e.g., controlled chemical modification and/or controlled backbone stereochemistry patterns, provide unexpected properties, including but not limited to those described herein. In some embodiments, provided compositions have improved properties, such as improved splicing-altering capabilities, lower toxicity, or improved protein binding profile, and/or improved delivery, etc. Particularly, in some embodiments, the present disclosure provides compositions and methods for improved delivery to target locations. Further, in some embodiments, the present disclosure provides compositions and methods for altering splicing of transcripts. In some embodiments, the present disclosure provides compositions and methods for improving splicing of transcripts. In some embodiments, altered transcript splicing by provided compositions and methods include production of products having desired and/or improved biological functions, and/or knockdown of undesired product by, e.g., modifying splicing products so that undesired biological functions can be suppressed or removed.

In some embodiments, a transcript is pre-mRNA. In some embodiments, a splicing product is mature RNA. In some embodiments, a splicing product is mRNA. In some embodiments, alteration comprises skipping one or more exons. In some embodiments, splicing of a transcript is improved in that exon skipping increases levels of mRNA and proteins that have improved beneficial activities compared with absence of exon skipping. In some embodiments, an exon causing frameshift is skipped. In some embodiments, an exon comprising an undesired mutation is skipped. In some embodiments, an exon comprising a premature termination codon is skipped. An undesired mutation can be a mutation causing changes in protein sequences; it can also be a silent mutation. In some embodiments, an exon comprising an undesired SNP is skipped.

In some embodiments, splicing of a transcript is improved in that exon skipping lowers levels of mRNA and proteins that have undesired activities compared with absence of exon skipping. In some embodiments, a target is knocked down through exon skipping which, by skipping one or more exons, causes premature stop codon and/or frameshift mutations.

Reading frame correction is achieved by skipping one or two exons flanking a deletion, by skipping in-frame exons containing a nonsense mutation, or by skipping duplicated exons.

In some embodiments, the present disclosure provides compositions and methods for reducing certain undesired repeats, such as CAG repeat (see, e.g., Evers, et al., Targeting several CAG expansion diseases by a single antisense oligonucleotide, PLoS One. 2011; 6(9):e24308. doi: 10.1371/journal.pone.0024308; Mulders, et al., Triplet-repeat oligonucleotide-mediated reversal of RNA toxicity in myotonic dystrophy, Proc Natl Acad Sci U.S.A. 2009 Aug. 18; 106(33):13915-20; etc.) by altering splicing, e.g., exon skipping. In some embodiments, example targets include but are not limited to: HTT (Huntingtin), ATXN3 (Ataxin 3), DMPK (dystrophia myotonica protein kinase), CNBP (Cellular Nucleic Acid Binding Protein), AR (Androgen Receptor), FOX01 (forkhead box protein 01), PCSK9 (proprotein convertase subtilisin/kexin type 9), TTR (transthyretin), AAT (alpha-1 antitrypsin), TMPRSS6 (transmembrane protease, serine 6), ALAS1 (aminolevulinate synthase 1), ATIII (antithrombin 3), FVII (factor VII), HAMP (hepcidin antimicrobial peptide), FXI (factor XI), FXII (factor XII), and PD-L1 (programmed death-ligand 1), APOC3 (Apolipoprotein C-III), PNPLA3 (patatin like phospholipase domain containing 3), and C9orf72. In some embodiments, targets include but are not limited to HTT, ATXN3, DMPK, CNBP, AR, C9ORF72 (target for familial frontotemporal dementia and amyotrophic lateral sclerosis) and those listed below:

| | | | | | | | |
|---|---|---|---|---|---|---|---|
| | | | | | Repeat | | |
| | | | Parent of | Repeat | number | Repeat | |
| | | | origin of | number | (pre- | number | Somatic |
| Disease | Sequence | Location | expansion | (normal) | mutation) | (disease) | instability |

Diseases with coding TNRs

| | | | | | | | |
|---|---|---|---|---|---|---|---|
| DRPLA | CAG | ATN1 (exon 5) | P | 6-35 | 35-48 | 49-88 | Yes |
| HD | CAG | HTT (exon 1) | P | 6-29 | 29-37 | 38-180 | Yes |
| OPMD | GCN | PABPN1 | P and M | 10 | 12-17 | >11 | None found in |
| | | (exon 1) | | | | | tissue tested |
| | | | | | | | (hypothalamus) |
| SCA1 | CAG | ATXN1 | P | 6-39 | 40 | 41-83 | Yes |
| | | (exon 8) | | | | | |
| SCA2 | CAG | ATXN2 | P | <31 | 31-32 | 32-200 | Unknown |
| | | (exon 1) | | | | | |
| SCA3 | CAG | ATXN3 | P | 12-40 | 41-85 | 52-86 | Unknown |
| (Machado- | | (exon 8) | | | | | |
| Joseph | | | | | | | |
| disease) | | | | | | | |
| SCA6 | CAG | CACNA1A | P | <18 | 19 | 20-33 | None found |
| | | (exon 47) | | | | | |
| SCA7 | CAG | ATXN7 | P | 4-17 | 28-33 | >36 | Yes |
| | | (exon 3) | | | | to >460 | |
| SCA17 | CAG | TBP (exon 3) | P > M | 25-42 | 43-48 | 45-66 | Yes |
| SMBA | CAG | AR (exon 1) | P | 13-31 | 32-39 | 40 | None found |

Diseases with non-coding TNRs

| | | | | | | | |
|---|---|---|---|---|---|---|---|
| DM1 | CTG | DMPK (3′ UTR) | M | 5-37 | 37-50 | <50 | Yes |
| DM2 | CCTG | CNBP | Uncertain | <30 | 31-74 | 75-11,000 | Yes |
| | | (intron 1) | | | | | |
| FRAX-E | GCC | AFF2 (5′ UTR) | M | 4-39 | 40-200 | >200 | Unknown |
| FRDA | GAA | FXN (intron 1) | Recessive | 5-30 | 31-100 | 70-1,000 | Yes |
| FXS | CGG | FMR1 (5′ UTR) | M | 6-50 | 55-200 | 200-4,000 | Yes |
| HDL2 | CTG | JPH3 (exon 2A) | M | 6-27 | 29-35 | 36-57 | Unknown |
| SCAB | CTG | ATXN8OS | M | 15-34 | 34-89 | 89-250 | Unknown |
| | | (3′ UTR) | | | | | |
| SCA10 | ATTCT | ATXN10 | M and P | 10-29 | 29-400 | 400-4,500 | Yes |
| | | (intron 9) | (smaller | | | | |
| | | | changes | | | | |
| | | | with M) | | | | |
| SCA12 | CAG | PPP2R2B | M and P | 7-28 | 28-66 | 66-78 | None found |
| | | (5′ UTR) | (more | | | | |
| | | | unstable | | | | |
| | | | with P) | | | | |

AFF2, AF4/FMR2 family, member 2; AR, androgen receptor;
ATN1, atrophin 1;
ATXN, ataxin;
ATXN8OS, ATXN8 opposite strand (non-protein coding);
CACNA1A, calcium channel, voltage-dependent, P/Q type, alpha 1A subunit;
CNBP, CCHC-type zinc finger nucleic acid binding protein;
DM, myotonic dystrophy; DMPK, dystrophia myotonica-protein kinase;
DRPLA, dentatorubral-pallidoluysian atrophy;
FMR1, fragile X mental retardation 1;
FRAX-E, mental retardation, X-linked, associated with FRAXE;
FRDA, Friedreich's ataxia; FXN, frataxin;
FXS, fragile X syndrome;
FXTAS, fragile X-associated tremor/ataxia syndrome;
HD, Huntington's disease;
HDL2, Huntington's disease-like 2;
HTT, huntingtin;
JPH3, junctophilin 3;
M, maternal;
OPMD, oculopharyngeal muscular dystrophy;
P, paternal;
PABPN1, poly(A) binding protein nuclear 1;
PPP2R2B, protein phosphatase 2, regulatory subunit B;
SCA, spinocerebellar ataxia;
SMBA, spinomuscular bulbar atrophy;
TBP, TATA-box binding protein;
TNR, trinucleotide repeat.

In some embodiments, provided oligonucleotides in provided compositions, e.g., oligonucleotides of a first plurality, comprise base modifications, sugar modifications, and/or internucleotidic linkage modifications. In some embodiments, provided oligonucleotides comprise base modifications and sugar modifications. In some embodiments, provided oligonucleotides comprise base modifications and internucleotidic linkage modifications. In some embodiments, provided oligonucleotides comprise sugar modifications and internucleotidic modifications. In some embodiments, provided compositions comprise base modifications, sugar modifications, and internucleotidic linkage modifications. Example chemical modifications, such as base modifications, sugar modifications, internucleotidic linkage modifications, etc. are widely known in the art including but not limited to those described in this disclosure. In some embodiments, a modified base is substituted A, T, C, G or U. In some embodiments, a sugar modification is 2′-modification. In some embodiments, a 2′-modification is 2′-R¹. In some embodiments, a 2′-modification is 2′-F modification. In some embodiments, a 2′-modification is 2′-OR¹. In some embodiments, a 2′-modification is 2′-OR¹, wherein R¹ is optionally substituted alkyl. In some embodiments, a 2′-modification is 2′-OMe. In some embodiments, a 2′-modification is 2′-MOE. In some embodiments, a modified sugar moiety is a bridged bicyclic or polycyclic ring. In some embodiments, a modified sugar moiety is a bridged bicyclic or polycyclic ring having 5-20 ring atoms wherein one or more ring atoms are optionally and independently heteroatoms. Example ring structures are widely known in the art, such as those found in BNA, LNA, etc. In some embodiments, provided oligonucleotides may comprise more than one types of sugar modifications; in some embodiments, provided oligonucleotides comprise both 2′-F and 2′-OR¹ modifications. In some embodiments, provided oligonucleotides comprise both 2′-F and 2′-OMe modifications. In some embodiments, provided oligonucleotides comprise both 2′-F and 2′-OMe modifications, and both phosphorothioate and natural phosphate linkages. In some embodiments, each chiral internucleotidic linkage, e.g., phosphorothioate linkage, is chirally controlled. In some embodiments, provided oligonucleotides comprise both one or more modified internucleotidic linkages and one or more natural phosphate linkages. In some embodiments, oligonucleotides comprising both modified internucleotidic linkage and natural phosphate linkage and compositions thereof provide improved properties, e.g., activities and toxicities, etc. In some embodiments, a modified internucleotidic linkage is a chiral internucleotidic linkage. In some embodiments, a modified internucleotidic linkage is a phosphorothioate linkage. In some embodiments, a modified internucleotidic linkage is a substituted phosphorothioate linkage.

Among other things, the present disclosure encompasses the recognition that stereorandom oligonucleotide preparations contain a plurality of distinct chemical entities that differ from one another, e.g., in the stereochemical structure of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence, they are different chemical entities at least due to their different backbone stereochemistry, and they can have, as demonstrated herein, different properties, e.g., activities, toxicities, etc. Among other things, the present disclosure provides new compositions that are or contain particular stereoisomers of oligonucleotides of interest. In some embodiments, a particular stereoisomer may be defined, for example, by its base sequence, its length, its pattern of backbone linkages, and its pattern of backbone chiral centers. As is understood in the art, in some embodiments, base sequence may refer to the identity and/or modification status of nucleoside residues (e.g., of sugar and/or base components, relative to standard naturally occurring nucleotides such as adenine, cytosine, guanosine, thymine, and uracil) in an oligonucleotide and/or to the hybridization character (i.e., the ability to hybridize with particular complementary residues) of such residues. In some embodiments, oligonucleotides in provided compositions comprise sugar modifications, e.g., 2′-modifications, at e.g., a wing region. In some embodiments, oligonucleotides in provided compositions comprise a region in the middle, e.g., a core region, that has no sugar modifications. In some embodiments, the present disclosure provide an oligonucleotide composition comprising a predetermined level of oligonucleotides of an individual oligonucleotide type which are chemically identical, e.g., they have the same base sequence, the same pattern of nucleoside modifications (modifications to sugar and base moieties, if any), the same pattern of backbone chiral centers, and the same pattern of backbone phosphorus modifications. The present disclosure demonstrates, among other things, that individual stereoisomers of a particular oligonucleotide can show different stability and/or activity (e.g., functional and/or toxicity properties) from each other. In some embodiments, property improvements achieved through inclusion and/or location of particular chiral structures within an oligonucleotide can be comparable to, or even better than those achieved through use of particular backbone linkages, residue modifications, etc. (e.g., through use of certain types of modified phosphates [e.g., phosphorothioate, substituted phosphorothioate, etc.], sugar modifications [e.g., 2′-modifications, etc.], and/or base modifications [e.g., methylation, etc.]). Among other things, the present disclosure recognizes that, in some embodiments, properties (e.g., activities, toxicities, etc.) of an oligonucleotide can be adjusted by optimizing its pattern of backbone chiral centers, optionally in combination with adjustment/optimization of one or more other features (e.g., linkage pattern, nucleoside modification pattern, etc.) of the oligonucleotide. As exemplified by various examples in the present disclosure, provided chirally controlled oligonucleotide compositions can demonstrate improved properties, such as lower toxicity, improved protein binding profile, improved delivery, etc.

In some embodiments, oligonucleotide properties can be adjusted by optimizing stereochemistry (pattern of backbone chiral centers) and chemical modifications (modifications of base, sugar, and/or internucleotidic linkage). Among other things, the present disclosure demonstrates that stereochemistry can further improve properties of oligonucleotides comprising chemical modifications. In some embodiments, the present disclosure provides oligonucleotide compositions wherein the oligonucleotides comprise nucleoside modifications, chiral internucleotidic linkages and natural phosphate linkages. For example, WV-1092 (mG*SmGmCmAmC*SA*SA*SG*SG*SG*SC*SA*SC*RA*SG*SmAmCmUmU*SmC (SEQ ID NO: 8)) comprises 2′-OMe modifications, phosphate and phosphorothioate linkages in its 5′- and 3′-wing regions, and phosphorothioate linkages in its core regions.

In some embodiments, the present disclosure provides oligonucleotide compositions which, unexpectedly, greatly improve properties of oligonucleotides. In some embodiments, provided oligonucleotide compositions provides surprisingly low toxicity. In some embodiments, provided oligonucleotide compositions provides surprisingly improved protein binding profile. In some embodiments, provided oligonucleotide compositions provides surprisingly enhanced delivery. In some embodiments, certain property improvement, such as lower toxicity, improved protein binding profile, and/or enhanced delivery, etc., are achieved without sacrificing other properties, e.g., activities, specificity, etc. In some embodiments, provided compositions provides lower toxicity, improved protein binding profile, and/or enhanced delivery, and improved activity, stability, and/or specificity (e.g., target-specificity, cleavage site specificity, etc.). Example improved activities (e.g., enhanced cleavage rates, increased target-specificity, cleavage site specificity, etc.) include but are not limited to those described in WO/2014/012081 and WO/2015/107425.

In some embodiments, a pattern of backbone chiral centers provides increased stability. In some embodiments, a pattern of backbone chiral centers provides surprisingly increased activity. In some embodiments, a pattern of backbone chiral centers provides increased stability and activity. In some embodiments, a pattern of backbone chiral centers provides surprisingly low toxicity. In some embodiments, a pattern of backbone chiral centers provides surprisingly low immune response. In some embodiments, a pattern of backbone chiral centers provides surprisingly low complement activation. In some embodiments, a pattern of backbone chiral centers provides surprisingly low complement activation via the alternative pathway. In some embodiments, a pattern of backbone chiral centers provides surprisingly improved protein binding profile. In some embodiments, a pattern of backbone chiral centers provides surprisingly increased binding to certain proteins. In some embodiments, a pattern of backbone chiral centers provides surprisingly enhanced delivery.

In some embodiments, a pattern of backbone chiral centers comprises or is (Sp)m(Rp)n, (Rp)n(Sp)m, (Np)t(Rp)n(Sp)m, or (Sp)t(Rp)n(Sp)m. In some embodiments, a pattern of backbone chiral centers comprises or is (Rp)n(Sp)m, (Np)t(Rp)n(Sp)m, or (Sp)t(Rp)n(Sp)m, wherein m>2. In some embodiments, a pattern of backbone chiral centers comprises or is (Rp)n(Sp)m, (Np)t(Rp)n(Sp)m, or (Sp)t(Rp)n(Sp)m, wherein n is 1, t>1, and m>2. In some embodiments, m>3. In some embodiments, m>4. In some embodiments, a pattern of backbone chiral centers comprises one or more achiral natural phosphate linkages.

In some embodiments, the present disclosure recognizes that chemical modifications, such as modifications of nucleosides and internucleotidic linkages, can provide enhanced properties. In some embodiments, the present disclosure demonstrates that combinations of chemical modifications and stereochemistry can provide unexpected, greatly improved properties (e.g., bioactivity, selectivity, etc.). In some embodiments, chemical combinations, such as modifications of sugars, bases, and/or internucleotidic linkages, are combined with stereochemistry patterns, e.g., (Rp)n(Sp)m, (Np)t(Rp)n(Sp)m, or (Sp)t(Rp)n(Sp)m, to provide oligonucleotides and compositions thereof with surprisingly enhanced properties. In some embodiments, a provided oligonucleotide composition is chirally controlled, and comprises a combination of 2′-modification of one or more sugar moieties, one or more natural phosphate linkages, one or more phosphorothioate linkages, and a stereochemistry pattern of (Rp)n(Sp)m, (Np)t(Rp)n(Sp)m, or (Sp)t(Rp)n(Sp)m, wherein m>2. In some embodiments, n is 1, t>1, and m>2. In some embodiments, m>3. In some embodiments, m>4.

In some embodiments, a pattern of backbone chiral centers comprises or is (Rp)n(Sp)m, (Sp)t(Rp)n, (Np)t(Rp)n(Sp)m, or (Sp)t(Rp)n(Sp)m. In some embodiments, a pattern of backbone chiral centers comprises or is (Rp)n(Sp)m. In some embodiments, a pattern of backbone chiral centers comprises or is (Sp)t(Rp)n. In some embodiments, a pattern of backbone chiral centers comprises or is (Np)t(Rp)n(Sp)m. In some embodiments, a pattern of backbone chiral centers comprises or is (Sp)t(Rp)n(Sp)m. In some embodiments, each of t and m is independently greater than 1, 2, 3, 4, 5, 6, 7, 8, 9, 10, 11, 12, 13, 14, 15, 16, 17, 18, 19 or 20. In some embodiments, each of t and m is independently greater than 1. In some embodiments, each of t and m is independently greater than 2. In some embodiments, each of t and m is independently greater than 2. In some embodiments, each of t and m is independently greater than 3. In some embodiments, each of t and m is independently greater than 4. In some embodiments, each of t and m is independently greater than 5. In some embodiments, each of t and m is independently greater than 6. In some embodiments, each of t and m is independently greater than 7. In some embodiments, each of t and m is independently greater than 8. In some embodiments, each of t and m is independently greater than 9. In some embodiments, each of t and m is independently greater than 10. In some embodiments, each of t and m is independently greater than 11. In some embodiments, each of t and m is independently greater than 12. In some embodiments, each of t and m is independently greater than 13. In some embodiments, each of t and m is independently greater than 14. In some embodiments, each of t and m is independently greater than 15. In some embodiments, t is greater than 1, 2, 3, 4, 5, 6, 7, 8, 9, 10, 11, 12, 13, 14, 15, 16, 17, 18, 19 or 20. In some embodiments, t is greater than 1. In some embodiments, t is greater than 2. In some embodiments, t is greater than 2. In some embodiments, t is greater than 3. In some embodiments, t is greater than 4. In some embodiments, t is greater than 5. In some embodiments, t is greater than 6. In some embodiments, t is greater than 7. In some embodiments, t is greater than 8. In some embodiments, t is greater than 9. In some embodiments, t is greater than 10. In some embodiments, t is greater than 11. In some embodiments, t is greater than 12. In some embodiments, t is greater than 13. In some embodiments, t is greater than 14. In some embodiments, t is greater than 15. In some embodiments, t is 1, 2, 3, 4, 5, 6, 7, 8, 9, 10, 11, 12, 13, 14, 15, 16, 17, 18, 19 or 20. In some embodiments, t is 1. In some embodiments, t is 2. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, t is 11. In some embodiments, t is 12. In some embodiments, t is 13. In some embodiments, t is 14. In some embodiments, t is 15. In some embodiments, m is greater than 1, 2, 3, 4, 5, 6, 7, 8, 9, 10, 11, 12, 13, 14, 15, 16, 17, 18, 19 or 20. In some embodiments, m is greater than 1. In some embodiments, m is greater than 2. In some embodiments, m is greater than 2. In some embodiments, m is greater than 3. In some embodiments, m is greater than 4. In some embodiments, m is greater than 5. In some embodiments, m is greater than 6. In some embodiments, m is greater than 7. In some embodiments, m is greater than 8. In some embodiments, m is greater than 9. In some embodiments, m is greater than 10. In some embodiments, m is greater than 11. In some embodiments, m is greater than 12. In some embodiments, m is greater than 13. In some embodiments, m is greater than 14. In some embodiments, m is greater than 15. In some embodiments, m is 1, 2, 3, 4, 5, 6, 7, 8, 9, 10, 11, 12, 13, 14, 15, 16, 17, 18, 19 or 20. In some embodiments, m is 1. In some embodiments, m is 2. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, t=m. In some embodiments, n is greater than 1, 2, 3, 4, 5, 6, 7, 8, 9, 10, 11, 12, 13, 14, 15, 16, 17, 18, 19 or 20. In some embodiments, n is greater than 1. In some embodiments, n is greater than 2. In some embodiments, n is greater than 2. In some embodiments, n is greater than 3. In some embodiments, n is greater than 4. In some embodiments, n is greater than 5. In some embodiments, n is greater than 6. In some embodiments, n is greater than 7. In some embodiments, n is greater than 8. In some embodiments, n is greater than 9. In some embodiments, n is greater than 10. In some embodiments, n is greater than 11. In some embodiments, n is greater than 12. In some embodiments, n is greater than 13. In some embodiments, n is greater than 14. In some embodiments, n is greater than 15. In some embodiments, n is 1, 2, 3, 4, 5, 6, 7, 8, 9, 10, 11, 12, 13, 14, 15, 16, 17, 18, 19 or 20. In some embodiments, n is 1. In some embodiments, n is 2. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15.

In some embodiments, provided oligonucleotides comprise one or more modified sugar moieties. In some embodiments, provided oligonucleotides comprise one or more modified sugar moieties. In some embodiments, provided oligonucleotides comprise 2 or more modified sugar moieties. In some embodiments, provided oligonucleotides comprise 3 or more modified sugar moieties. In some embodiments, provided oligonucleotides comprise 4 or more modified sugar moieties. In some embodiments, provided oligonucleotides comprise 5 or more modified sugar moieties. In some embodiments, provided oligonucleotides comprise 6 or more modified sugar moieties. In some embodiments, provided oligonucleotides comprise 7 or more modified sugar moieties. In some embodiments, provided oligonucleotides comprise 8 or more modified sugar moieties. In some embodiments, provided oligonucleotides comprise 9 or more modified sugar moieties. In some embodiments, provided oligonucleotides comprise 10 or more modified sugar moieties. In some embodiments, provided oligonucleotides comprise 15 or more modified sugar moieties. In some embodiments, provided oligonucleotides comprise 20 or more modified sugar moieties. In some embodiments, provided oligonucleotides comprise 25 or more modified sugar moieties. In some embodiments, provided oligonucleotides comprise 30 or more modified sugar moieties.

Provided oligonucleotides can comprise various number of chiral internucleotidic linkages. In some embodiments, provided oligonucleotides comprise no chiral internucleotidic linkages. In some embodiments, provided oligonucleotides comprise one chiral internucleotidic linkage. In some embodiments, provided oligonucleotides comprise 2 or more chiral internucleotidic linkages. In some embodiments, provided oligonucleotides comprise 3 or more chiral internucleotidic linkages. In some embodiments, provided oligonucleotides comprise 4 or more chiral internucleotidic linkages. In some embodiments, provided oligonucleotides comprise 5 or more chiral internucleotidic linkages. In some embodiments, provided oligonucleotides comprise 6 or more chiral internucleotidic linkages. In some embodiments, provided oligonucleotides comprise 7 or more chiral internucleotidic linkages. In some embodiments, provided oligonucleotides comprise 8 or more chiral internucleotidic linkages. In some embodiments, provided oligonucleotides comprise 9 or more chiral internucleotidic linkages. In some embodiments, provided oligonucleotides comprise 10 or more chiral internucleotidic linkages. In some embodiments, provided oligonucleotides comprise 15 or more chiral internucleotidic linkages. In some embodiments, provided oligonucleotides comprise 20 or more chiral internucleotidic linkages. In some embodiments, provided oligonucleotides comprise 25 or more chiral internucleotidic linkages. In some embodiments, provided oligonucleotides comprise 30 or more chiral internucleotidic linkages.

Provided oligonucleotides can comprise various number of achiral internucleotidic linkages. In some embodiments, provided oligonucleotides comprise no achiral internucleotidic linkages. In some embodiments, provided oligonucleotides comprise one achiral internucleotidic linkage. In some embodiments, provided oligonucleotides comprise 2 or more achiral internucleotidic linkages. In some embodiments, provided oligonucleotides comprise 3 or more achiral internucleotidic linkages. In some embodiments, provided oligonucleotides comprise 4 or more achiral internucleotidic linkages. In some embodiments, provided oligonucleotides comprise 5 or more achiral internucleotidic linkages. In some embodiments, provided oligonucleotides comprise 6 or more achiral internucleotidic linkages. In some embodiments, provided oligonucleotides comprise 7 or more achiral internucleotidic linkages. In some embodiments, provided oligonucleotides comprise 8 or more achiral internucleotidic linkages. In some embodiments, provided oligonucleotides comprise 9 or more achiral internucleotidic linkages. In some embodiments, provided oligonucleotides comprise 10 or more achiral internucleotidic linkages. In some embodiments, provided oligonucleotides comprise 15 or more achiral internucleotidic linkages. In some embodiments, provided oligonucleotides comprise 20 or more achiral internucleotidic linkages. In some embodiments, provided oligonucleotides comprise 25 or more achiral internucleotidic linkages. In some embodiments, provided oligonucleotides comprise 30 or more achiral internucleotidic linkages.

In some embodiments, 5% or more of the sugar moieties of provided oligonucleotides are modified. In some embodiments, 10% or more of the sugar moieties of provided oligonucleotides are modified. In some embodiments, 15% or more of the sugar moieties of provided oligonucleotides are modified. In some embodiments, 20% or more of the sugar moieties of provided oligonucleotides are modified. In some embodiments, 25% or more of the sugar moieties of provided oligonucleotides are modified. In some embodiments, 30% or more of the sugar moieties of provided oligonucleotides are modified. In some embodiments, 35% or more of the sugar moieties of provided oligonucleotides are modified. In some embodiments, 40% or more of the sugar moieties of provided oligonucleotides are modified. In some embodiments, 45% or more of the sugar moieties of provided oligonucleotides are modified. In some embodiments, 50% or more of the sugar moieties of provided oligonucleotides are modified. In some embodiments, 55% or more of the sugar moieties of provided oligonucleotides are modified. In some embodiments, 60% or more of the sugar moieties of provided oligonucleotides are modified. In some embodiments, 65% or more of the sugar moieties of provided oligonucleotides are modified. In some embodiments, 70% or more of the sugar moieties of provided oligonucleotides are modified. In some embodiments, 75% or more of the sugar moieties of provided oligonucleotides are modified. In some embodiments, 80% or more of the sugar moieties of provided oligonucleotides are modified. In some embodiments, 85% or more of the sugar moieties of provided oligonucleotides are modified. In some embodiments, 90% or more of the sugar moieties of provided oligonucleotides are modified. In some embodiments, 95% or more of the sugar moieties of provided oligonucleotides are modified. In some embodiments, each sugar moiety of provided oligonucleotides is modified.

In some embodiments, provided oligonucleotides comprise one or more 2′-F. In some embodiments, provided oligonucleotides comprise two or more 2′-F. In some embodiments, provided oligonucleotides comprise three or more 2′-F. In some embodiments, provided oligonucleotides comprise four or more 2′-F. In some embodiments, provided oligonucleotides comprise five or more 2′-F. In some embodiments, provided oligonucleotides comprise six or more 2′-F. In some embodiments, provided oligonucleotides comprise seven or more 2′-F. In some embodiments, provided oligonucleotides comprise eight or more 2′-F. In some embodiments, provided oligonucleotides comprise nine or more 2′-F. In some embodiments, provided oligonucleotides comprise ten or more 2′-F. In some embodiments, provided oligonucleotides comprise 11 or more 2′-F. In some embodiments, provided oligonucleotides comprise 12 or more 2′-F. In some embodiments, provided oligonucleotides comprise 13 or more 2′-F. In some embodiments, provided oligonucleotides comprise 14 or more 2′-F. In some embodiments, provided oligonucleotides comprise 15 or more 2′-F. In some embodiments, provided oligonucleotides comprise 16 or more 2′-F. In some embodiments, provided oligonucleotides comprise 17 or more 2′-F. In some embodiments, provided oligonucleotides comprise 18 or more 2′-F. In some embodiments, provided oligonucleotides comprise 19 or more 2′-F. In some embodiments, provided oligonucleotides comprise 20 or more 2′-F. In some embodiments, provided oligonucleotides comprise 21 or more 2′-F. In some embodiments, provided oligonucleotides comprise 22 or more 2′-F. In some embodiments, provided oligonucleotides comprise 23 or more 2′-F. In some embodiments, provided oligonucleotides comprise 24 or more 2′-F. In some embodiments, provided oligonucleotides comprise 25 or more 2′-F. In some embodiments, provided oligonucleotides comprise 30 or more 2′-F. In some embodiments, provided oligonucleotides comprise 35 or more 2′-F.

In some embodiments, provided oligonucleotides comprise one 2′-F. In some embodiments, provided oligonucleotides comprise two 2′-F. In some embodiments, provided oligonucleotides comprise three 2′-F. In some embodiments, provided oligonucleotides comprise four 2′-F. In some embodiments, provided oligonucleotides comprise five 2′-F. In some embodiments, provided oligonucleotides comprise six 2′-F. In some embodiments, provided oligonucleotides comprise seven 2′-F. In some embodiments, provided oligonucleotides comprise eight 2′-F. In some embodiments, provided oligonucleotides comprise nine 2′-F. In some embodiments, provided oligonucleotides comprise ten 2′-F. In some embodiments, provided oligonucleotides comprise 11 2′-F. In some embodiments, provided oligonucleotides comprise 12 2′-F. In some embodiments, provided oligonucleotides comprise 13 2′-F. In some embodiments, provided oligonucleotides comprise 14 2′-F. In some embodiments, provided oligonucleotides comprise 15 2′-F. In some embodiments, provided oligonucleotides comprise 16 2′-F. In some embodiments, provided oligonucleotides comprise 17 2′-F. In some embodiments, provided oligonucleotides comprise 18 2′-F. In some embodiments, provided oligonucleotides comprise 19 2′-F. In some embodiments, provided oligonucleotides comprise 20 2′-F. In some embodiments, provided oligonucleotides comprise 21 2′-F. In some embodiments, provided oligonucleotides comprise 22 2′-F. In some embodiments, provided oligonucleotides comprise 23 2′-F. In some embodiments, provided oligonucleotides comprise 24 2′-F. In some embodiments, provided oligonucleotides comprise 25 2′-F. In some embodiments, provided oligonucleotides comprise 30 2′-F. In some embodiments, provided oligonucleotides comprise 35 2′-F.

In some embodiments, provided oligonucleotides comprise one or more consecutive 2′-F. In some embodiments, provided oligonucleotides comprise two or more consecutive 2′-F. In some embodiments, provided oligonucleotides comprise three or more consecutive 2′-F. In some embodiments, provided oligonucleotides comprise four or more consecutive 2′-F. In some embodiments, provided oligonucleotides comprise five or more consecutive 2′-F. In some embodiments, provided oligonucleotides comprise six or more consecutive 2′-F. In some embodiments, provided oligonucleotides comprise seven or more consecutive 2′-F. In some embodiments, provided oligonucleotides comprise eight or more consecutive 2′-F. In some embodiments, provided oligonucleotides comprise nine or more consecutive 2′-F. In some embodiments, provided oligonucleotides comprise ten or more consecutive 2′-F. In some embodiments, provided oligonucleotides comprise 11 or more consecutive 2′-F. In some embodiments, provided oligonucleotides comprise 12 or more consecutive 2′-F. In some embodiments, provided oligonucleotides comprise 13 or more consecutive 2′-F. In some embodiments, provided oligonucleotides comprise 14 or more consecutive 2′-F. In some embodiments, provided oligonucleotides comprise 15 or more consecutive 2′-F. In some embodiments, provided oligonucleotides comprise 16 or more consecutive 2′-F. In some embodiments, provided oligonucleotides comprise 17 or more consecutive 2′-F. In some embodiments, provided oligonucleotides comprise 18 or more consecutive 2′-F. In some embodiments, provided oligonucleotides comprise 19 or more consecutive 2′-F. In some embodiments, provided oligonucleotides comprise 20 or more consecutive 2′-F. In some embodiments, provided oligonucleotides comprise 21 or more consecutive 2′-F. In some embodiments, provided oligonucleotides comprise 22 or more consecutive 2′-F. In some embodiments, provided oligonucleotides comprise 23 or more consecutive 2′-F. In some embodiments, provided oligonucleotides comprise 24 or more consecutive 2′-F. In some embodiments, provided oligonucleotides comprise 25 or more consecutive 2′-F. In some embodiments, provided oligonucleotides comprise 30 or more consecutive 2′-F. In some embodiments, provided oligonucleotides comprise 35 or more consecutive 2′-F.

In some embodiments, provided oligonucleotides comprise one consecutive 2′-F. In some embodiments, provided oligonucleotides comprise two consecutive 2′-F. In some embodiments, provided oligonucleotides comprise three consecutive 2′-F. In some embodiments, provided oligonucleotides comprise four consecutive 2′-F. In some embodiments, provided oligonucleotides comprise five consecutive 2′-F. In some embodiments, provided oligonucleotides comprise six consecutive 2′-F. In some embodiments, provided oligonucleotides comprise seven consecutive 2′-F. In some embodiments, provided oligonucleotides comprise eight consecutive 2′-F. In some embodiments, provided oligonucleotides comprise nine consecutive 2′-F. In some embodiments, provided oligonucleotides comprise ten consecutive 2′-F. In some embodiments, provided oligonucleotides comprise 11 consecutive 2′-F. In some embodiments, provided oligonucleotides comprise 12 consecutive 2′-F. In some embodiments, provided oligonucleotides comprise 13 consecutive 2′-F. In some embodiments, provided oligonucleotides comprise 14 consecutive 2′-F. In some embodiments, provided oligonucleotides comprise 15 consecutive 2′-F. In some embodiments, provided oligonucleotides comprise 16 consecutive 2′-F. In some embodiments, provided oligonucleotides comprise 17 consecutive 2′-F. In some embodiments, provided oligonucleotides comprise 18 consecutive 2′-F. In some embodiments, provided oligonucleotides comprise 19 consecutive 2′-F. In some embodiments, provided oligonucleotides comprise 20 consecutive 2′-F. In some embodiments, provided oligonucleotides comprise 21 consecutive 2′-F. In some embodiments, provided oligonucleotides comprise 22 consecutive 2′-F. In some embodiments, provided oligonucleotides comprise 23 consecutive 2′-F. In some embodiments, provided oligonucleotides comprise 24 consecutive 2′-F. In some embodiments, provided oligonucleotides comprise 25 consecutive 2′-F. In some embodiments, provided oligonucleotides comprise 30 consecutive 2′-F. In some embodiments, provided oligonucleotides comprise 35 consecutive 2′-F.

In some embodiments, a nucleoside comprising a 2′-modification is followed by a modified internucleotidic linkage. In some embodiments, a nucleoside comprising a 2′-modification is preceded by a modified internucleotidic linkage. In some embodiments, a modified internucleotidic linkage is a chiral internucleotidic linkage. In some embodiments, a modified internucleotidic linkage is a phosphorothioate. In some embodiments, a chiral internucleotidic linkage is Sp. In some embodiments, a nucleoside comprising a 2′-modification is followed by an Sp chiral internucleotidic linkage. In some embodiments, a nucleoside comprising a 2′-F is followed by an Sp chiral internucleotidic linkage. In some embodiments, a nucleoside comprising a 2′-modification is preceded by an Sp chiral internucleotidic linkage. In some embodiments, a nucleoside comprising a 2′-F is preceded by an Sp chiral internucleotidic linkage. In some embodiments, a chiral internucleotidic linkage is Rp. In some embodiments, a nucleoside comprising a 2′-modification is followed by an Rp chiral internucleotidic linkage. In some embodiments, a nucleoside comprising a 2′-F is followed by an Rp chiral internucleotidic linkage. In some embodiments, a nucleoside comprising a 2′-modification is preceded by an Rp chiral internucleotidic linkage. In some embodiments, a nucleoside comprising a 2′-Fis preceded by an Rp chiral internucleotidic linkage.

In some embodiments, provided oligonucleotides comprise one or more natural phosphate linkages and one or more modified internucleotidic linkages.

Provided oligonucleotides can comprise various number of natural phosphate linkages. In some embodiments, provided oligonucleotides comprise no natural phosphate linkages. In some embodiments, provided oligonucleotides comprise one natural phosphate linkage. In some embodiments, provided oligonucleotides comprise 2 or more natural phosphate linkages. In some embodiments, provided oligonucleotides comprise 3 or more natural phosphate linkages. In some embodiments, provided oligonucleotides comprise 4 or more natural phosphate linkages. In some embodiments, provided oligonucleotides comprise 5 or more natural phosphate linkages. In some embodiments, provided oligonucleotides comprise 6 or more natural phosphate linkages. In some embodiments, provided oligonucleotides comprise 7 or more natural phosphate linkages. In some embodiments, provided oligonucleotides comprise 8 or more natural phosphate linkages. In some embodiments, provided oligonucleotides comprise 9 or more natural phosphate linkages. In some embodiments, provided oligonucleotides comprise 10 or more natural phosphate linkages. In some embodiments, provided oligonucleotides comprise 15 or more natural phosphate linkages. In some embodiments, provided oligonucleotides comprise 20 or more natural phosphate linkages. In some embodiments, provided oligonucleotides comprise 25 or more natural phosphate linkages. In some embodiments, provided oligonucleotides comprise 30 or more natural phosphate linkages.

Provided oligonucleotides can comprise various numbers of consecutive chiral internucleotidic linkages. In some embodiments, provided oligonucleotides comprise no consecutive chiral internucleotidic linkages. In some embodiments, provided oligonucleotides comprise one consecutive chiral internucleotidic linkage. In some embodiments, provided oligonucleotides comprise 2 or more consecutive chiral internucleotidic linkages. In some embodiments, provided oligonucleotides comprise 3 or more consecutive chiral internucleotidic linkages. In some embodiments, provided oligonucleotides comprise 4 or more consecutive chiral internucleotidic linkages. In some embodiments, provided oligonucleotides comprise 5 or more consecutive chiral internucleotidic linkages. In some embodiments, provided oligonucleotides comprise 6 or more consecutive chiral internucleotidic linkages. In some embodiments, provided oligonucleotides comprise 7 or more consecutive chiral internucleotidic linkages. In some embodiments, provided oligonucleotides comprise 8 or more consecutive chiral internucleotidic linkages. In some embodiments, provided oligonucleotides comprise 9 or more consecutive chiral internucleotidic linkages. In some embodiments, provided oligonucleotides comprise 10 or more consecutive chiral internucleotidic linkages. In some embodiments, provided oligonucleotides comprise 15 or more consecutive chiral internucleotidic linkages. In some embodiments, provided oligonucleotides comprise 20 or more consecutive chiral internucleotidic linkages. In some embodiments, provided oligonucleotides comprise 25 or more consecutive chiral internucleotidic linkages. In some embodiments, provided oligonucleotides comprise 30 or more consecutive chiral internucleotidic linkages.

Provided oligonucleotides can comprise various numbers of consecutive achiral internucleotidic linkages. In some embodiments, provided oligonucleotides comprise no consecutive achiral internucleotidic linkages. In some embodiments, provided oligonucleotides comprise one consecutive achiral internucleotidic linkage. In some embodiments, provided oligonucleotides comprise 2 or more consecutive achiral internucleotidic linkages. In some embodiments, provided oligonucleotides comprise 3 or more consecutive achiral internucleotidic linkages. In some embodiments, provided oligonucleotides comprise 4 or more consecutive achiral internucleotidic linkages. In some embodiments, provided oligonucleotides comprise 5 or more consecutive achiral internucleotidic linkages. In some embodiments, provided oligonucleotides comprise 6 or more consecutive achiral internucleotidic linkages. In some embodiments, provided oligonucleotides comprise 7 or more consecutive achiral internucleotidic linkages. In some embodiments, provided oligonucleotides comprise 8 or more consecutive achiral internucleotidic linkages. In some embodiments, provided oligonucleotides comprise 9 or more consecutive achiral internucleotidic linkages. In some embodiments, provided oligonucleotides comprise 10 or more consecutive achiral internucleotidic linkages. In some embodiments, provided oligonucleotides comprise 15 or more consecutive achiral internucleotidic linkages. In some embodiments, provided oligonucleotides comprise 20 or more consecutive achiral internucleotidic linkages. In some embodiments, provided oligonucleotides comprise 25 or more consecutive achiral internucleotidic linkages. In some embodiments, provided oligonucleotides comprise 30 or more consecutive achiral internucleotidic linkages.

In some embodiments, 5% or more of the internucleotidic linkages of provided oligonucleotides are natural phosphate linkages. In some embodiments, 10% or more of the internucleotidic linkages of provided oligonucleotides are natural phosphate linkages. In some embodiments, 15% or more of the internucleotidic linkages of provided oligonucleotides are natural phosphate linkages. In some embodiments, 20% or more of the internucleotidic linkages of provided oligonucleotides are natural phosphate linkages. In some embodiments, 25% or more of the internucleotidic linkages of provided oligonucleotides are natural phosphate linkages. In some embodiments, 30% or more of the internucleotidic linkages of provided oligonucleotides are natural phosphate linkages. In some embodiments, 35% or more of the internucleotidic linkages of provided oligonucleotides are natural phosphate linkages. In some embodiments, 40% or more of the internucleotidic linkages of provided oligonucleotides are natural phosphate linkages. In some embodiments, 45% or more of the internucleotidic linkages of provided oligonucleotides are natural phosphate linkages. In some embodiments, 50% or more of the internucleotidic linkages of provided oligonucleotides are natural phosphate linkages. In some embodiments, 55% or more of the internucleotidic linkages of provided oligonucleotides are natural phosphate linkages. In some embodiments, 60% or more of the internucleotidic linkages of provided oligonucleotides are natural phosphate linkages. In some embodiments, 65% or more of the internucleotidic linkages of provided oligonucleotides are natural phosphate linkages. In some embodiments, 70% or more of the internucleotidic linkages of provided oligonucleotides are natural phosphate linkages. In some embodiments, 75% or more of the internucleotidic linkages of provided oligonucleotides are natural phosphate linkages. In some embodiments, 80% or more of the internucleotidic linkages of provided oligonucleotides are natural phosphate linkages. In some embodiments, 85% or more of the internucleotidic linkages of provided oligonucleotides are natural phosphate linkages. In some embodiments, 90% or more of the internucleotidic linkages of provided oligonucleotides are natural phosphate linkages. In some embodiments, 95% or more of the internucleotidic linkages of provided oligonucleotides are natural phosphate linkages.

In some embodiments, provided oligonucleotides comprise no more than about 25 consecutive unmodified sugar moieties. In some embodiments, provided oligonucleotides comprise no more than about 20 consecutive unmodified sugar moieties. In some embodiments, provided oligonucleotides comprise no more than about 15 consecutive unmodified sugar moieties. In some embodiments, provided oligonucleotides comprise no more than about 10 consecutive unmodified sugar moieties. In some embodiments, provided oligonucleotides comprise no more than about 9 consecutive unmodified sugar moieties. In some embodiments, provided oligonucleotides comprise no more than about 8 consecutive unmodified sugar moieties. In some embodiments, provided oligonucleotides comprise no more than about 7 consecutive unmodified sugar moieties. In some embodiments, provided oligonucleotides comprise no more than about 6 consecutive unmodified sugar moieties. In some embodiments, provided oligonucleotides comprise no more than about 5 consecutive unmodified sugar moieties. In some embodiments, provided oligonucleotides comprise no more than about 4 consecutive unmodified sugar moieties. In some embodiments, provided oligonucleotides comprise no more than about 3 consecutive unmodified sugar moieties. In some embodiments, provided oligonucleotides comprise no more than about 2 consecutive unmodified sugar moieties. In some embodiments, provided oligonucleotides comprise no more than about 25 unmodified sugar moieties. In some embodiments, provided oligonucleotides comprise no more than about 20 unmodified sugar moieties. In some embodiments, provided oligonucleotides comprise no more than about 15 unmodified sugar moieties. In some embodiments, provided oligonucleotides comprise no more than about 10 unmodified sugar moieties. In some embodiments, provided oligonucleotides comprise no more than about 5 unmodified sugar moieties.

In some embodiments, provided oligonucleotides comprise no more than about 95% unmodified sugar moieties. In some embodiments, provided oligonucleotides comprise no more than about 90% unmodified sugar moieties. In some embodiments, provided oligonucleotides comprise no more than about 85% unmodified sugar moieties. In some embodiments, provided oligonucleotides comprise no more than about 80% unmodified sugar moieties. In some embodiments, provided oligonucleotides comprise no more than about 70% unmodified sugar moieties. In some embodiments, provided oligonucleotides comprise no more than about 60% unmodified sugar moieties. In some embodiments, provided oligonucleotides comprise no more than about 50% unmodified sugar moieties. In some embodiments, provided oligonucleotides comprise no more than about 40% unmodified sugar moieties. In some embodiments, provided oligonucleotides comprise no more than about 30% unmodified sugar moieties. In some embodiments, provided oligonucleotides comprise no more than about 20% unmodified sugar moieties. In some embodiments, provided oligonucleotides comprise no more than about 10% unmodified sugar moieties. In some embodiments, provided oligonucleotides comprise no more than about 5% unmodified sugar moieties. In some embodiments, provided oligonucleotides comprise no more than about 15 consecutive unmodified sugar moieties. In some embodiments, provided oligonucleotides comprise no more than about 10 consecutive unmodified sugar moieties. In some embodiments, provided oligonucleotides comprise no more than about 9 consecutive unmodified sugar moieties. In some embodiments, provided oligonucleotides comprise no more than about 8 consecutive unmodified sugar moieties. In some embodiments, provided oligonucleotides comprise no more than about 7 consecutive unmodified sugar moieties. In some embodiments, provided oligonucleotides comprise no more than about 6 consecutive unmodified sugar moieties. In some embodiments, provided oligonucleotides comprise no more than about 5 consecutive unmodified sugar moieties. In some embodiments, provided oligonucleotides comprise no more than about 4 consecutive unmodified sugar moieties. In some embodiments, provided oligonucleotides comprise no more than about 3 consecutive unmodified sugar moieties. In some embodiments, provided oligonucleotides comprise no more than about 2 consecutive unmodified sugar moieties. In some embodiments, provided oligonucleotides comprise no more than about 25 unmodified sugar moieties. In some embodiments, provided oligonucleotides comprise no more than about 20 unmodified sugar moieties. In some embodiments, provided oligonucleotides comprise no more than about 15 unmodified sugar moieties. In some embodiments, provided oligonucleotides comprise no more than about 10 unmodified sugar moieties. In some embodiments, provided oligonucleotides comprise no more than about 5 unmodified sugar moieties.

In some embodiments, provided oligonucleotides comprise no more than about 95% unmodified sugar moieties. In some embodiments, provided oligonucleotides comprise no more than about 90% unmodified sugar moieties. In some embodiments, provided oligonucleotides comprise no more than about 85% unmodified sugar moieties. In some embodiments, provided oligonucleotides comprise no more than about 80% unmodified sugar moieties. In some embodiments, provided oligonucleotides comprise no more than about 70% unmodified sugar moieties. In some embodiments, provided oligonucleotides comprise no more than about 60% unmodified sugar moieties. In some embodiments, provided oligonucleotides comprise no more than about 50% unmodified sugar moieties. In some embodiments, provided oligonucleotides comprise no more than about 40% unmodified sugar moieties. In some embodiments, provided oligonucleotides comprise no more than about 30% unmodified sugar moieties. In some embodiments, provided oligonucleotides comprise no more than about 20% unmodified sugar moieties. In some embodiments, provided oligonucleotides comprise no more than about 10% unmodified sugar moieties. In some embodiments, provided oligonucleotides comprise no more than about 5% unmodified sugar moieties. In some embodiments, each sugar moiety of the oligonucleotides of the first plurality is independently modified.

In some embodiments, provided oligonucleotides comprise two or more modified internucleotidic linkages. In some embodiments, provided oligonucleotides comprise three or more modified internucleotidic linkages. In some embodiments, provided oligonucleotides comprise four or more modified internucleotidic linkages. In some embodiments, provided oligonucleotides comprise five or more modified internucleotidic linkages. In some embodiments, provided oligonucleotides comprise ten or more modified internucleotidic linkages. In some embodiments, provided oligonucleotides comprise about 15 or more modified internucleotidic linkages. In some embodiments, provided oligonucleotides comprise about 20 or more modified internucleotidic linkages. In some embodiments, provided oligonucleotides comprise about 25 or more modified internucleotidic linkages.

In some embodiments, about 5% of the internucleotidic linkages in provided oligonucleotides are modified internucleotidic linkages. In some embodiments, about 10% of the internucleotidic linkages in provided oligonucleotides are modified internucleotidic linkages. In some embodiments, about 20% of the internucleotidic linkages in provided oligonucleotides are modified internucleotidic linkages. In some embodiments, about 30% of the internucleotidic linkages in provided oligonucleotides are modified internucleotidic linkages. In some embodiments, about 40% of the internucleotidic linkages in provided oligonucleotides are modified internucleotidic linkages. In some embodiments, about 50% of the internucleotidic linkages in provided oligonucleotides are modified internucleotidic linkages. In some embodiments, about 60% of the internucleotidic linkages in provided oligonucleotides are modified internucleotidic linkages. In some embodiments, about 70% of the internucleotidic linkages in provided oligonucleotides are modified internucleotidic linkages. In some embodiments, about 80% of the internucleotidic linkages in provided oligonucleotides are modified internucleotidic linkages. In some embodiments, about 85% of the internucleotidic linkages in provided oligonucleotides are modified internucleotidic linkages. In some embodiments, about 90% of the internucleotidic linkages in provided oligonucleotides are modified internucleotidic linkages. In some embodiments, about 95% of the internucleotidic linkages in provided oligonucleotides are modified internucleotidic linkages.

In some embodiments, provided oligonucleotides comprise no more than about 25 consecutive natural phosphate linkages. In some embodiments, provided oligonucleotides comprise no more than about 20 consecutive natural phosphate linkages. In some embodiments, provided oligonucleotides comprise no more than about 15 consecutive natural phosphate linkages. In some embodiments, provided oligonucleotides comprise no more than about 10 consecutive natural phosphate linkages. In some embodiments, provided oligonucleotides comprise no more than about 9 consecutive natural phosphate linkages. In some embodiments, provided oligonucleotides comprise no more than about 8 consecutive natural phosphate linkages. In some embodiments, provided oligonucleotides comprise no more than about 7 consecutive natural phosphate linkages. In some embodiments, provided oligonucleotides comprise no more than about 6 consecutive natural phosphate linkages. In some embodiments, provided oligonucleotides comprise no more than about 5 consecutive natural phosphate linkages. In some embodiments, provided oligonucleotides comprise no more than about 4 consecutive natural phosphate linkages. In some embodiments, provided oligonucleotides comprise no more than about 3 consecutive natural phosphate linkages. In some embodiments, provided oligonucleotides comprise no more than about 2 consecutive natural phosphate linkages. In some embodiments, provided oligonucleotides comprise no more than about 25 natural phosphate linkages. In some embodiments, provided oligonucleotides comprise no more than about 20 natural phosphate linkages. In some embodiments, provided oligonucleotides comprise no more than about 15 natural phosphate linkages. In some embodiments, provided oligonucleotides comprise no more than about 10 natural phosphate linkages. In some embodiments, provided oligonucleotides comprise no more than about 5 natural phosphate linkages. In some embodiments, provided oligonucleotides comprise no more than about 95% natural phosphate linkages. In some embodiments, provided oligonucleotides comprise no more than about 90% natural phosphate linkages. In some embodiments, provided oligonucleotides comprise no more than about 85% natural phosphate linkages. In some embodiments, provided oligonucleotides comprise no more than about 80% natural phosphate linkages. In some embodiments, provided oligonucleotides comprise no more than about 70% natural phosphate linkages. In some embodiments, provided oligonucleotides comprise no more than about 60% natural phosphate linkages. In some embodiments, provided oligonucleotides comprise no more than about 50% natural phosphate linkages. In some embodiments, provided oligonucleotides comprise no more than about 40% natural phosphate linkages. In some embodiments, provided oligonucleotides comprise no more than about 30% natural phosphate linkages. In some embodiments, provided oligonucleotides comprise no more than about 20% natural phosphate linkages. In some embodiments, provided oligonucleotides comprise no more than about 10% natural phosphate linkages. In some embodiments, provided oligonucleotides comprise no more than about 5% natural phosphate linkages.

In some embodiments, provided oligonucleotides comprise no DNA nucleotide.

A DNA nucleotide is a nucleotide in which the sugar moiety is an unmodified DNA sugar moiety, and the internucleotidic linkage is a natural phosphate linkage. In some embodiments, provided oligonucleotides comprise no more than 2 DNA nucleotides. In some embodiments, provided oligonucleotides comprise no more than 3 DNA nucleotides. In some embodiments, provided oligonucleotides comprise no more than 4 DNA nucleotides. In some embodiments, provided oligonucleotides comprise no more than 5 DNA nucleotides. In some embodiments, provided oligonucleotides comprise no more than 6 DNA nucleotides. In some embodiments, provided oligonucleotides comprise no more than 7 DNA nucleotides. In some embodiments, provided oligonucleotides comprise no more than 8 DNA nucleotides. In some embodiments, provided oligonucleotides comprise no more than 9 DNA nucleotides. In some embodiments, provided oligonucleotides comprise no more than 10 DNA nucleotides. In some embodiments, provided oligonucleotides comprise no more than 11 DNA nucleotides. In some embodiments, provided oligonucleotides comprise no more than 12 DNA nucleotides. In some embodiments, provided oligonucleotides comprise no more than 13 DNA nucleotides. In some embodiments, provided oligonucleotides comprise no more than 14 DNA nucleotides. In some embodiments, provided oligonucleotides comprise no more than 15 DNA nucleotides. In some embodiments, provided oligonucleotides comprise no more than 20 DNA nucleotides. In some embodiments, provided oligonucleotides comprise no more than 25 DNA nucleotides. In some embodiments, provided oligonucleotides comprise no more than 30 DNA nucleotides.

In some embodiments, provided oligonucleotides comprise no more than 2 consecutive DNA nucleotides. In some embodiments, provided oligonucleotides comprise no more than 3 consecutive DNA nucleotides. In some embodiments, provided oligonucleotides comprise no more than 4 consecutive DNA nucleotides. In some embodiments, provided oligonucleotides comprise no more than 5 consecutive DNA nucleotides. In some embodiments, provided oligonucleotides comprise no more than 6 consecutive DNA nucleotides. In some embodiments, provided oligonucleotides comprise no more than 7 consecutive DNA nucleotides. In some embodiments, provided oligonucleotides comprise no more than 8 consecutive DNA nucleotides. In some embodiments, provided oligonucleotides comprise no more than 9 consecutive DNA nucleotides. In some embodiments, provided oligonucleotides comprise no more than 10 consecutive DNA nucleotides. In some embodiments, provided oligonucleotides comprise no more than 11 consecutive DNA nucleotides. In some embodiments, provided oligonucleotides comprise no more than 12 consecutive DNA nucleotides. In some embodiments, provided oligonucleotides comprise no more than 13 consecutive DNA nucleotides. In some embodiments, provided oligonucleotides comprise no more than 14 consecutive DNA nucleotides. In some embodiments, provided oligonucleotides comprise no more than 15 consecutive DNA nucleotides. In some embodiments, provided oligonucleotides comprise no more than 20 consecutive DNA nucleotides. In some embodiments, provided oligonucleotides comprise no more than 25 consecutive DNA nucleotides. In some embodiments, provided oligonucleotides comprise no more than 30 consecutive DNA nucleotides.

In some embodiments, compared to a reference condition, provided oligonucleotide compositions are surprisingly effective. In some embodiments, desired biological effects (e.g., as measured by increased levels of desired mRNA, proteins, etc., decreased levels of undesired mRNA, proteins, etc., delivery to target locations, etc.) can be enhanced by more than 5, 10, 15, 20, 25, 30, 40, 50, or 100 folds. In some embodiments, a change is measured by increase of a desired mRNA level compared to a reference condition. In some embodiments, a change is measured by decrease of an undesired mRNA level compared to a reference condition. In some embodiments, a change is measured by increase of delivery to target locations compared to a reference condition. In some embodiments, a reference condition is absence of oligonucleotide treatment. In some embodiments, a reference condition is a stereorandom composition of oligonucleotides having the same base sequence and chemical modifications.

In some embodiments, a desired biological effect is enhanced by more than 2 folds. In some embodiments, a desired biological effect is enhanced by more than 3 folds. In some embodiments, a desired biological effect is enhanced by more than 4 folds. In some embodiments, a desired biological effect is enhanced by more than 5 folds. In some embodiments, a desired biological effect is enhanced by more than 6 folds. In some embodiments, a desired biological effect is enhanced by more than 7 folds. In some embodiments, a desired biological effect is enhanced by more than 8 folds. In some embodiments, a desired biological effect is enhanced by more than 9 folds. In some embodiments, a desired biological effect is enhanced by more than 10 folds. In some embodiments, a desired biological effect is enhanced by more than 11 folds. In some embodiments, a desired biological effect is enhanced by more than 12 folds. In some embodiments, a desired biological effect is enhanced by more than 13 folds. In some embodiments, a desired biological effect is enhanced by more than 14 folds. In some embodiments, a desired biological effect is enhanced by more than 15 folds. In some embodiments, a desired biological effect is enhanced by more than 20 folds. In some embodiments, a desired biological effect is enhanced by more than 25 folds. In some embodiments, a desired biological effect is enhanced by more than 30 folds. In some embodiments, a desired biological effect is enhanced by more than 35 folds. In some embodiments, a desired biological effect is enhanced by more than 40 folds. In some embodiments, a desired biological effect is enhanced by more than 45 folds. In some embodiments, a desired biological effect is enhanced by more than 50 folds. In some embodiments, a desired biological effect is enhanced by more than 60 folds. In some embodiments, a desired biological effect is enhanced by more than 70 folds. In some embodiments, a desired biological effect is enhanced by more than 80 folds. In some embodiments, a desired biological effect is enhanced by more than 90 folds. In some embodiments, a desired biological effect is enhanced by more than 100 folds. In some embodiments, a desired biological effect is enhanced by more than 200 folds. In some embodiments, a desired biological effect is enhanced by more than 500 folds.

In some embodiments, provided oligonucleotides comprise two wing and one core regions. In some embodiments, provided oligonucleotides comprises a 5′-wing-core-wing-3′ structure. In some embodiments, provided oligonucleotides are of a 5′-wing-core-wing-3′ gapmer structure. In some embodiments, the two wing regions are identical. In some embodiments, the two wing regions are different. In some embodiments, the two wing regions are identical in chemical modifications. In some embodiments, the two wing regions are identical in 2′-modifications. In some embodiments, the two wing regions are identical in internucleotidic linkage modifications. In some embodiments, the two wing regions are identical in patterns of backbone chiral centers. In some embodiments, the two wing regions are identical in pattern of backbone linkages. In some embodiments, the two wing regions are identical in pattern of backbone linkage types. In some embodiments, the two wing regions are identical in pattern of backbone phosphorus modifications.

In some embodiments, provided oligonucleotides comprise one wing and one core regions. In some embodiments, provided oligonucleotides comprises a 5′-wing-core-3′ hemimer structure. In some embodiments, provided oligonucleotides are of a 5′-wing-core-3′ hemimer structure. In some embodiments, provided oligonucleotides comprises a 5′-core-wing-3′ hemimer structure. In some embodiments, provided oligonucleotides are of a 5′-core-wing-3′ hemimer structure.

A wing region can be differentiated from a core region in that a wing region contains a different structure feature than a core region. For example, in some embodiments, a wing region differs from a core region in that they have different sugar modifications, base modifications, internucleotidic linkages, internucleotidic linkage stereochemistry, etc. In some embodiments, a wing region differs from a core region in that they have different 2′-modifications of the sugars.

In some embodiments, an internucleotidic linkage between a wing region and a core region is considered part of the wing region. In some embodiments, an internucleotidic linkage between a 5′-wing region and a core region is considered part of the wing region. In some embodiments, an internucleotidic linkage between a 3′-wing region and a core region is considered part of the wing region. In some embodiments, an internucleotidic linkage between a wing region and a core region is considered part of the core region. In some embodiments, an internucleotidic linkage between a 5′-wing region and a core region is considered part of the core region. In some embodiments, an internucleotidic linkage between a 3′-wing region and a core region is considered part of the core region.

In some embodiments, an internucleotidic linkage between a wing region and a core region is considered part of the wing region. In some embodiments, an internucleotidic linkage between a 5′-wing region and a core region is considered part of the wing region. In some embodiments, an internucleotidic linkage between a 3′-wing region and a core region is considered part of the wing region. In some embodiments, an internucleotidic linkage between a wing region and a core region is considered part of the core region. In some embodiments, an internucleotidic linkage between a 5′-wing region and a core region is considered part of the core region. In some embodiments, an internucleotidic linkage between a 3′-wing region and a core region is considered part of the core region.

In some embodiments, a wing region comprises 2 or more nucleosides. In some embodiments, a wing region comprises 3 or more nucleosides. In some embodiments, a wing region comprises 4 or more nucleosides. In some embodiments, a wing region comprises 5 or more nucleosides. In some embodiments, a wing region comprises 6 or more nucleosides. In some embodiments, a wing region comprises 7 or more nucleosides. In some embodiments, a wing region comprises 8 or more nucleosides. In some embodiments, a wing region comprises 9 or more nucleosides. In some embodiments, a wing region comprises 10 or more nucleosides. In some embodiments, a wing region comprises 11 or more nucleosides. In some embodiments, a wing region comprises 12 or more nucleosides. In some embodiments, a wing region comprises 13 or more nucleosides. In some embodiments, a wing region comprises 14 or more nucleosides. In some embodiments, a wing region comprises 15 or more nucleosides.

In some embodiments, a wing region comprises 2 or more modified internucleotidic linkages. In some embodiments, a wing region comprises 3 or more modified internucleotidic linkages. In some embodiments, a wing region comprises 4 or more modified internucleotidic linkages. In some embodiments, a wing region comprises 5 or more modified internucleotidic linkages. In some embodiments, a wing region comprises 6 or more modified internucleotidic linkages. In some embodiments, a wing region comprises 7 or more modified internucleotidic linkages. In some embodiments, a wing region comprises 8 or more modified internucleotidic linkages. In some embodiments, a wing region comprises 9 or more modified internucleotidic linkages. In some embodiments, a wing region comprises 10 or more modified internucleotidic linkages. In some embodiments, a wing region comprises 11 or more modified internucleotidic linkages. In some embodiments, a wing region comprises 12 or more modified internucleotidic linkages. In some embodiments, a wing region comprises 13 or more modified internucleotidic linkages. In some embodiments, a wing region comprises 14 or more modified internucleotidic linkages. In some embodiments, a wing region comprises 15 or more modified internucleotidic linkages.

In some embodiments, a chiral internucleotidic linkage or a modified internucleotidic linkage has the structure of formula I. In some embodiments, a chiral internucleotidic linkage or a modified internucleotidic linkage is phosphorothioate. In some embodiments, each chiral internucleotidic linkage or a modified internucleotidic linkage independently has the structure of formula I. In some embodiments, each chiral internucleotidic linkage or a modified internucleotidic linkage is phosphorothioate.

In some embodiments, a wing region comprises 2 or more consecutive modified internucleotidic linkages. In some embodiments, a wing region comprises 3 or more consecutive modified internucleotidic linkages. In some embodiments, a wing region comprises 4 or more consecutive modified internucleotidic linkages. In some embodiments, a wing region comprises 5 or more consecutive modified internucleotidic linkages. In some embodiments, a wing region comprises 6 or more consecutive modified internucleotidic linkages. In some embodiments, a wing region comprises 7 or more consecutive modified internucleotidic linkages. In some embodiments, a wing region comprises 8 or more consecutive modified internucleotidic linkages. In some embodiments, a wing region comprises 9 or more consecutive modified internucleotidic linkages. In some embodiments, a wing region comprises 10 or more consecutive modified internucleotidic linkages. In some embodiments, a wing region comprises 11 or more consecutive modified internucleotidic linkages. In some embodiments, a wing region comprises 12 or more consecutive modified internucleotidic linkages. In some embodiments, a wing region comprises 13 or more consecutive modified internucleotidic linkages. In some embodiments, a wing region comprises 14 or more consecutive modified internucleotidic linkages. In some embodiments, a wing region comprises 15 or more consecutive modified internucleotidic linkages. In some embodiments, each internucleotidic linkage in a wing region is independently a modified internucleotidic linkage.

In some embodiments, 5% or more of the internucleotidic linkages of provided oligonucleotides are modified internucleotidic linkages. In some embodiments, 10% or more of the internucleotidic linkages of provided oligonucleotides are modified internucleotidic linkages. In some embodiments, 15% or more of the internucleotidic linkages of provided oligonucleotides are modified internucleotidic linkages. In some embodiments, 20% or more of the internucleotidic linkages of provided oligonucleotides are modified internucleotidic linkages. In some embodiments, 25% or more of the internucleotidic linkages of provided oligonucleotides are modified internucleotidic linkages. In some embodiments, 30% or more of the internucleotidic linkages of provided oligonucleotides are modified internucleotidic linkages. In some embodiments, 35% or more of the internucleotidic linkages of provided oligonucleotides are modified internucleotidic linkages. In some embodiments, 40% or more of the internucleotidic linkages of provided oligonucleotides are modified internucleotidic linkages. In some embodiments, 45% or more of the internucleotidic linkages of a wing region are modified internucleotidic linkages. In some embodiments, 50% or more of the internucleotidic linkages of a wing region are modified internucleotidic linkages. In some embodiments, 55% or more of the internucleotidic linkages of a wing region are modified internucleotidic linkages. In some embodiments, 60% or more of the internucleotidic linkages of a wing region are modified internucleotidic linkages. In some embodiments, 65% or more of the internucleotidic linkages of a wing region are modified internucleotidic linkages. In some embodiments, 70% or more of the internucleotidic linkages of a wing region are modified internucleotidic linkages. In some embodiments, 75% or more of the internucleotidic linkages of a wing region are modified internucleotidic linkages. In some embodiments, 80% or more of the internucleotidic linkages of a wing region are modified internucleotidic linkages. In some embodiments, 85% or more of the internucleotidic linkages of a wing region are modified internucleotidic linkages. In some embodiments, 90% or more of the internucleotidic linkages of a wing region are modified internucleotidic linkages. In some embodiments, 95% or more of the internucleotidic linkages of a wing region are modified internucleotidic linkages. In some embodiments, each internucleotidic linkage of a wing region is a modified internucleotidic linkage.

In some embodiments, a wing region comprises 2 or more natural phosphate linkages. In some embodiments, a wing region comprises 3 or more natural phosphate linkages. In some embodiments, a wing region comprises 4 or more natural phosphate linkages. In some embodiments, a wing region comprises 5 or more natural phosphate linkages. In some embodiments, a wing region comprises 6 or more natural phosphate linkages. In some embodiments, a wing region comprises 7 or more natural phosphate linkages. In some embodiments, a wing region comprises 8 or more natural phosphate linkages. In some embodiments, a wing region comprises 9 or more natural phosphate linkages. In some embodiments, a wing region comprises 10 or more natural phosphate linkages. In some embodiments, a wing region comprises 11 or more natural phosphate linkages. In some embodiments, a wing region comprises 12 or more natural phosphate linkages. In some embodiments, a wing region comprises 13 or more natural phosphate linkages. In some embodiments, a wing region comprises 14 or more natural phosphate linkages. In some embodiments, a wing region comprises 15 or more natural phosphate linkages. In some embodiments, a wing region comprises 2 or more consecutive natural phosphate linkages. In some embodiments, a wing region comprises 3 or more consecutive natural phosphate linkages. In some embodiments, a wing region comprises 4 or more consecutive natural phosphate linkages. In some embodiments, a wing region comprises 5 or more consecutive natural phosphate linkages. In some embodiments, a wing region comprises 6 or more consecutive natural phosphate linkages. In some embodiments, a wing region comprises 7 or more consecutive natural phosphate linkages. In some embodiments, a wing region comprises 8 or more consecutive natural phosphate linkages. In some embodiments, a wing region comprises 9 or more consecutive natural phosphate linkages. In some embodiments, a wing region comprises 10 or more consecutive natural phosphate linkages. In some embodiments, a wing region comprises 11 or more consecutive natural phosphate linkages. In some embodiments, a wing region comprises 12 or more consecutive natural phosphate linkages. In some embodiments, a wing region comprises 13 or more consecutive natural phosphate linkages. In some embodiments, a wing region comprises 14 or more consecutive natural phosphate linkages. In some embodiments, a wing region comprises 15 or more consecutive natural phosphate linkages. In some embodiments, each internucleotidic linkage in a wing region is independently a natural phosphate linkage.

In some embodiments, 5% or more of the internucleotidic linkages of provided oligonucleotides are natural phosphate linkages. In some embodiments, 10% or more of the internucleotidic linkages of provided oligonucleotides are natural phosphate linkages. In some embodiments, 15% or more of the internucleotidic linkages of provided oligonucleotides are natural phosphate linkages. In some embodiments, 20% or more of the internucleotidic linkages of provided oligonucleotides are natural phosphate linkages. In some embodiments, 25% or more of the internucleotidic linkages of provided oligonucleotides are natural phosphate linkages. In some embodiments, 30% or more of the internucleotidic linkages of provided oligonucleotides are natural phosphate linkages. In some embodiments, 35% or more of the internucleotidic linkages of provided oligonucleotides are natural phosphate linkages. In some embodiments, 40% or more of the internucleotidic linkages of provided oligonucleotides are natural phosphate linkages. In some embodiments, 45% or more of the internucleotidic linkages of a wing region are natural phosphate linkages. In some embodiments, 50% or more of the internucleotidic linkages of a wing region are natural phosphate linkages. In some embodiments, 55% or more of the internucleotidic linkages of a wing region are natural phosphate linkages. In some embodiments, 60% or more of the internucleotidic linkages of a wing region are natural phosphate linkages. In some embodiments, 65% or more of the internucleotidic linkages of a wing region are natural phosphate linkages. In some embodiments, 70% or more of the internucleotidic linkages of a wing region are natural phosphate linkages. In some embodiments, 75% or more of the internucleotidic linkages of a wing region are natural phosphate linkages. In some embodiments, 80% or more of the internucleotidic linkages of a wing region are natural phosphate linkages. In some embodiments, 85% or more of the internucleotidic linkages of a wing region are natural phosphate linkages. In some embodiments, 90% or more of the internucleotidic linkages of a wing region are natural phosphate linkages. In some embodiments, 95% or more of the internucleotidic linkages of a wing region are natural phosphate linkages. In some embodiments, each internucleotidic linkage of a wing region is a natural phosphate linkage.

In some embodiments, a core region comprises 2 or more modified internucleotidic linkages. In some embodiments, a core region comprises 3 or more modified internucleotidic linkages. In some embodiments, a core region comprises 4 or more modified internucleotidic linkages. In some embodiments, a core region comprises 5 or more modified internucleotidic linkages. In some embodiments, a core region comprises 6 or more modified internucleotidic linkages. In some embodiments, a core region comprises 7 or more modified internucleotidic linkages. In some embodiments, a core region comprises 8 or more modified internucleotidic linkages. In some embodiments, a core region comprises 9 or more modified internucleotidic linkages. In some embodiments, a core region comprises 10 or more modified internucleotidic linkages. In some embodiments, a core region comprises 11 or more modified internucleotidic linkages. In some embodiments, a core region comprises 12 or more modified internucleotidic linkages. In some embodiments, a core region comprises 13 or more modified internucleotidic linkages. In some embodiments, a core region comprises 14 or more modified internucleotidic linkages. In some embodiments, a core region comprises 15 or more modified internucleotidic linkages. In some embodiments, a core region comprises 2 or more consecutive modified internucleotidic linkages. In some embodiments, a core region comprises 3 or more consecutive modified internucleotidic linkages. In some embodiments, a core region comprises 4 or more consecutive modified internucleotidic linkages. In some embodiments, a core region comprises 5 or more consecutive modified internucleotidic linkages. In some embodiments, a core region comprises 6 or more consecutive modified internucleotidic linkages. In some embodiments, a core region comprises 7 or more consecutive modified internucleotidic linkages. In some embodiments, a core region comprises 8 or more consecutive modified internucleotidic linkages. In some embodiments, a core region comprises 9 or more consecutive modified internucleotidic linkages. In some embodiments, a core region comprises 10 or more consecutive modified internucleotidic linkages. In some embodiments, a core region comprises 11 or more consecutive modified internucleotidic linkages. In some embodiments, a core region comprises 12 or more consecutive modified internucleotidic linkages. In some embodiments, a core region comprises 13 or more consecutive modified internucleotidic linkages. In some embodiments, a core region comprises 14 or more consecutive modified internucleotidic linkages. In some embodiments, a core region comprises 15 or more consecutive modified internucleotidic linkages. In some embodiments, each internucleotidic linkage in a core region is independently a modified internucleotidic linkage.

In some embodiments, 5% or more of the internucleotidic linkages of provided oligonucleotides are modified internucleotidic linkages. In some embodiments, 10% or more of the internucleotidic linkages of provided oligonucleotides are modified internucleotidic linkages. In some embodiments, 15% or more of the internucleotidic linkages of provided oligonucleotides are modified internucleotidic linkages. In some embodiments, 20% or more of the internucleotidic linkages of provided oligonucleotides are modified internucleotidic linkages. In some embodiments, 25% or more of the internucleotidic linkages of provided oligonucleotides are modified internucleotidic linkages. In some embodiments, 30% or more of the internucleotidic linkages of provided oligonucleotides are modified internucleotidic linkages. In some embodiments, 35% or more of the internucleotidic linkages of provided oligonucleotides are modified internucleotidic linkages. In some embodiments, 40% or more of the internucleotidic linkages of provided oligonucleotides are modified internucleotidic linkages. In some embodiments, 45% or more of the internucleotidic linkages of a core region are modified internucleotidic linkages. In some embodiments, 50% or more of the internucleotidic linkages of a core region are modified internucleotidic linkages. In some embodiments, 55% or more of the internucleotidic linkages of a core region are modified internucleotidic linkages. In some embodiments, 60% or more of the internucleotidic linkages of a core region are modified internucleotidic linkages. In some embodiments, 65% or more of the internucleotidic linkages of a core region are modified internucleotidic linkages. In some embodiments, 70% or more of the internucleotidic linkages of a core region are modified internucleotidic linkages. In some embodiments, 75% or more of the internucleotidic linkages of a core region are modified internucleotidic linkages. In some embodiments, 80% or more of the internucleotidic linkages of a core region are modified internucleotidic linkages. In some embodiments, 85% or more of the internucleotidic linkages of a core region are modified internucleotidic linkages. In some embodiments, 90% or more of the internucleotidic linkages of a core region are modified internucleotidic linkages. In some embodiments, 95% or more of the internucleotidic linkages of a core region are modified internucleotidic linkages. In some embodiments, each internucleotidic linkage of a core region is a modified internucleotidic linkage.

Provided oligonucleotides can comprise various number of modified internucleotidic linkages. In some embodiments, provided oligonucleotides comprise one modified internucleotidic linkage. In some embodiments, provided oligonucleotides comprise 2 or more modified internucleotidic linkages. In some embodiments, provided oligonucleotides comprise 3 or more modified internucleotidic linkages. In some embodiments, provided oligonucleotides comprise 4 or more modified internucleotidic linkages. In some embodiments, provided oligonucleotides comprise 5 or more modified internucleotidic linkages. In some embodiments, provided oligonucleotides comprise 6 or more modified internucleotidic linkages. In some embodiments, provided oligonucleotides comprise 7 or more modified internucleotidic linkages. In some embodiments, provided oligonucleotides comprise 8 or more modified internucleotidic linkages. In some embodiments, provided oligonucleotides comprise 9 or more modified internucleotidic linkages. In some embodiments, provided oligonucleotides comprise 10 or more modified internucleotidic linkages. In some embodiments, provided oligonucleotides comprise 15 or more modified internucleotidic linkages. In some embodiments, provided oligonucleotides comprise 20 or more modified internucleotidic linkages. In some embodiments, provided oligonucleotides comprise 25 or more modified internucleotidic linkages. In some embodiments, provided oligonucleotides comprise 30 or more modified internucleotidic linkages.

In some embodiments, 5% or more of the internucleotidic linkages of provided oligonucleotides are modified internucleotidic linkages. In some embodiments, 10% or more of the internucleotidic linkages of provided oligonucleotides are modified internucleotidic linkages. In some embodiments, 15% or more of the internucleotidic linkages of provided oligonucleotides are modified internucleotidic linkages. In some embodiments, 20% or more of the internucleotidic linkages of provided oligonucleotides are modified internucleotidic linkages. In some embodiments, 25% or more of the internucleotidic linkages of provided oligonucleotides are modified internucleotidic linkages. In some embodiments, 30% or more of the internucleotidic linkages of provided oligonucleotides are modified internucleotidic linkages. In some embodiments, 35% or more of the internucleotidic linkages of provided oligonucleotides are modified internucleotidic linkages. In some embodiments, 40% or more of the internucleotidic linkages of provided oligonucleotides are modified internucleotidic linkages. In some embodiments, 45% or more of the internucleotidic linkages of provided oligonucleotides are modified internucleotidic linkages. In some embodiments, 50% or more of the internucleotidic linkages of provided oligonucleotides are modified internucleotidic linkages. In some embodiments, 55% or more of the internucleotidic linkages of provided oligonucleotides are modified internucleotidic linkages. In some embodiments, 60% or more of the internucleotidic linkages of provided oligonucleotides are modified internucleotidic linkages. In some embodiments, 65% or more of the internucleotidic linkages of provided oligonucleotides are modified internucleotidic linkages. In some embodiments, 70% or more of the internucleotidic linkages of provided oligonucleotides are modified internucleotidic linkages. In some embodiments, 75% or more of the internucleotidic linkages of provided oligonucleotides are modified internucleotidic linkages. In some embodiments, 80% or more of the internucleotidic linkages of provided oligonucleotides are modified internucleotidic linkages. In some embodiments, 85% or more of the internucleotidic linkages of provided oligonucleotides are modified internucleotidic linkages. In some embodiments, 90% or more of the internucleotidic linkages of provided oligonucleotides are modified internucleotidic linkages. In some embodiments, 95% or more of the internucleotidic linkages of provided oligonucleotides are modified internucleotidic linkages. In some embodiments, each internucleotidic linkage of provided oligonucleotides is a modified internucleotidic linkage.

In some embodiments, the present disclosure provides a chirally controlled oligonucleotide composition comprising one or more lipids, and a first plurality of oligonucleotides defined by having:
1) a common base sequence and length;
2) a common pattern of backbone linkages; and
3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers;

wherein the lipids are optionally and independently conjugated to one or more oligonucleotides of the plurality.

In some embodiments, a common base sequence and length may be referred to as a common base sequence. In some embodiments, oligonucleotides having a common base sequence may have the same pattern of nucleoside modifications, e.g., sugar modifications, base modifications, etc. In some embodiments, a pattern of nucleoside modifications may be represented by a combination of locations and modifications.

As understood by a person having ordinary skill in the art, a stereorandom or racemic preparation of oligonucleotides is prepared by non-stereoselective and/or low-stereoselective coupling of nucleotide monomers, typically without using any chiral auxiliaries, chiral modification reagents, and/or chiral catalysts. In some embodiments, in a substantially racemic (or chirally uncontrolled) preparation of oligonucleotides, all or most coupling steps are not chirally controlled in that the coupling steps are not specifically conducted to provide enhanced stereoselectivity. An example substantially racemic preparation of oligonucleotides is the preparation of phosphorothioate oligonucleotides through sulfurizing phosphite triesters from commonly used phosphoramidite oligonucleotide synthesis with either tteraethylthiuram disulfide or (TETD) or 3H-1, 2-bensodithiol-3-one 1, 1-dioxide (BDTD), a well-known process in the art. In some embodiments, substantially racemic preparation of oligonucleotides provides substantially racemic oligonucleotide compositions (or chirally uncontrolled oligonucleotide compositions). In some embodiments, at least one coupling of a nucleotide monomer has a diastereoselectivity lower than about 60:40, 70:30, 80:20, 85:15, 90:10, 91:9, 92:8, 97:3, 98:2, or 99:1. In some embodiments, at least two couplings of a nucleotide monomer have a diastereoselectivity lower than about 60:40, 70:30, 80:20, 85:15, 90:10, 91:9, 92:8, 97:3, 98:2, or 99:1. In some embodiments, at least three couplings of a nucleotide monomer have a diastereoselectivity lower than about 60:40, 70:30, 80:20, 85:15, 90:10, 91:9, 92:8, 97:3, 98:2, or 99:1. In some embodiments, at least four couplings of a nucleotide monomer have a diastereoselectivity lower than about 60:40, 70:30, 80:20, 85:15, 90:10, 91:9, 92:8, 97:3, 98:2, or 99:1. In some embodiments, at least five couplings of a nucleotide monomer have a diastereoselectivity lower than about 60:40, 70:30, 80:20, 85:15, 90:10, 91:9, 92:8, 97:3, 98:2, or 99:1. In some embodiments, each coupling of a nucleotide monomer independently has a diastereoselectivity lower than about 60:40, 70:30, 80:20, 85:15, 90:10, 91:9, 92:8, 97:3, 98:2, or 99:1. In some embodiments, in a stereorandom or racemic preparations, at least one internucleotidic linkage has a diastereoselectivity lower than about 60:40, 70:30, 80:20, 85:15, 90:10, 91:9, 92:8, 97:3, 98:2, or 99:1. In some embodiments, at least two internucleotidic linkages have a diastereoselectivity lower than about 60:40, 70:30, 80:20, 85:15, 90:10, 91:9, 92:8, 97:3, 98:2, or 99:1. In some embodiments, at least three internucleotidic linkages have a diastereoselectivity lower than about 60:40, 70:30, 80:20, 85:15, 90:10, 91:9, 92:8, 97:3, 98:2, or 99:1. In some embodiments, at least four internucleotidic linkages have a diastereoselectivity lower than about 60:40, 70:30, 80:20, 85:15, 90:10, 91:9, 92:8, 97:3, 98:2, or 99:1. In some embodiments, at least five internucleotidic linkages have a diastereoselectivity lower than about 60:40, 70:30, 80:20, 85:15, 90:10, 91:9, 92:8, 97:3, 98:2, or 99:1. In some embodiments, each internucleotidic linkage independently has a diastereoselectivity lower than about 60:40, 70:30, 80:20, 85:15, 90:10, 91:9, 92:8, 97:3, 98:2, or 99:1. In some embodiments, a diastereoselectivity is lower than about 60:40. In some embodiments, a diastereoselectivity is lower than about 70:30. In some embodiments, a diastereoselectivity is lower than about 80:20. In some embodiments, a diastereoselectivity is lower than about 90:10. In some embodiments, a diastereoselectivity is lower than about 91:9. In some embodiments, a diastereoselectivity is lower than about 92:8. In some embodiments, a diastereoselectivity is lower than about 93:7. In some embodiments, a diastereoselectivity is lower than about 94:6. In some embodiments, a diastereoselectivity is lower than about 95:5. In some embodiments, a diastereoselectivity is lower than about 96:4. In some embodiments, a diastereoselectivity is lower than about 97:3. In some embodiments, a diastereoselectivity is lower than about 98:2. In some embodiments, a diastereoselectivity is lower than about 99:1. In some embodiments, at least one coupling has a diastereoselectivity lower than about 90:10. In some embodiments, at least two couplings have a diastereoselectivity lower than about 90:10. In some embodiments, at least three couplings have a diastereoselectivity lower than about 90:10. In some embodiments, at least four couplings have a diastereoselectivity lower than about 90:10. In some embodiments, at least five couplings have a diastereoselectivity lower than about 90:10. In some embodiments, each coupling independently has a diastereoselectivity lower than about 90:10. In some embodiments, at least one internucleotidic linkage has a diastereoselectivity lower than about 90:10. In some embodiments, at least two internucleotidic linkages have a diastereoselectivity lower than about 90:10. In some embodiments, at least three internucleotidic linkages have a diastereoselectivity lower than about 90:10. In some embodiments, at least four internucleotidic linkages have a diastereoselectivity lower than about 90:10. In some embodiments, at least five internucleotidic linkages have a diastereoselectivity lower than about 90:10. In some embodiments, each internucleotidic linkage independently has a diastereoselectivity lower than about 90:10.

As understood by a person having ordinary skill in the art, in some embodiments, diastereoselectivity of a coupling or a linkage can be assessed through the diastereoselectivity of a dimer formation under the same or comparable conditions, wherein the dimer has the same 5′- and 3′-nucleosides and internucleotidic linkage. For example, diastereoselectivity of the underlined coupling or linkage in NNNNNNNG*SGNNNNNNN can be assessed from coupling two G moieties under the same or comparable conditions, e.g., monomers, chiral auxiliaries, solvents, activators, temperatures, etc.

In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have identical structures.

In some embodiments, oligonucleotides of an oligonucleotide type have a common pattern of backbone phosphorus modifications and a common pattern of sugar modifications. In some embodiments, oligonucleotides of an oligonucleotide type have a common pattern of backbone phosphorus modifications and a common pattern of base modifications. In some embodiments, oligonucleotides of an oligonucleotide type have a common pattern of backbone phosphorus modifications and a common pattern of nucleoside modifications. In some embodiments, oligonucleotides of an oligonucleotide type are identical.

In some embodiments, a chirally controlled oligonucleotide composition is a substantially pure preparation of an oligonucleotide type in that oligonucleotides in the composition that are not of the oligonucleotide type are impurities form the preparation process of said oligonucleotide type, in some case, after certain purification procedures.

In some embodiments, at least about 20% of the oligonucleotides in the composition have a common base sequence and length, a common pattern of backbone linkages, and a common pattern of backbone chiral centers. In some embodiments, at least about 25% of the oligonucleotides in the composition have a common base sequence and length, a common pattern of backbone linkages, and a common pattern of backbone chiral centers. In some embodiments, at least about 30% of the oligonucleotides in the composition have a common base sequence and length, a common pattern of backbone linkages, and a common pattern of backbone chiral centers. In some embodiments, at least about 35% of the oligonucleotides in the composition have a common base sequence and length, a common pattern of backbone linkages, and a common pattern of backbone chiral centers. In some embodiments, at least about 40% of the oligonucleotides in the composition have a common base sequence and length, a common pattern of backbone linkages, and a common pattern of backbone chiral centers. In some embodiments, at least about 45% of the oligonucleotides in the composition have a common base sequence and length, a common pattern of backbone linkages, and a common pattern of backbone chiral centers. In some embodiments, at least about 50% of the oligonucleotides in the composition have a common base sequence and length, a common pattern of backbone linkages, and a common pattern of backbone chiral centers. In some embodiments, at least about 55% of the oligonucleotides in the composition have a common base sequence and length, a common pattern of backbone linkages, and a common pattern of backbone chiral centers. In some embodiments, at least about 60% of the oligonucleotides in the composition have a common base sequence and length, a common pattern of backbone linkages, and a common pattern of backbone chiral centers. In some embodiments, at least about 65% of the oligonucleotides in the composition have a common base sequence and length, a common pattern of backbone linkages, and a common pattern of backbone chiral centers. In some embodiments, at least about 70% of the oligonucleotides in the composition have a common base sequence and length, a common pattern of backbone linkages, and a common pattern of backbone chiral centers. In some embodiments, at least about 75% of the oligonucleotides in the composition have a common base sequence and length, a common pattern of backbone linkages, and a common pattern of backbone chiral centers. In some embodiments, at least about 80% of the oligonucleotides in the composition have a common base sequence and length, a common pattern of backbone linkages, and a common pattern of backbone chiral centers. In some embodiments, at least about 85% of the oligonucleotides in the composition have a common base sequence and length, a common pattern of backbone linkages, and a common pattern of backbone chiral centers. In some embodiments, at least about 90% of the oligonucleotides in the composition have a common base sequence and length, a common pattern of backbone linkages, and a common pattern of backbone chiral centers. In some embodiments, at least about 92% of the oligonucleotides in the composition have a common base sequence and length, a common pattern of backbone linkages, and a common pattern of backbone chiral centers. In some embodiments, at least about 94% of the oligonucleotides in the composition have a common base sequence and length, a common pattern of backbone linkages, and a common pattern of backbone chiral centers. In some embodiments, at least about 95% of the oligonucleotides in the composition have a common base sequence and length, a common pattern of backbone linkages, and a common pattern of backbone chiral centers. In some embodiments, at least about 91%, 92%, 93%, 94%, 95%, 96%, 97%, 98%, or 99% of the oligonucleotides in the composition have a common base sequence and length, a common pattern of backbone linkages, and a common pattern of backbone chiral centers. In some embodiments, greater than about 99% of the oligonucleotides in the composition have a common base sequence and length, a common pattern of backbone linkages, and a common pattern of backbone chiral centers. In some embodiments, purity of a chirally controlled oligonucleotide composition of an oligonucleotide can be expressed as the percentage of oligonucleotides in the composition that have a common base sequence and length, a common pattern of backbone linkages, and a common pattern of backbone chiral centers.

In some embodiments, oligonucleotides having a common base sequence and length, a common pattern of backbone linkages, and a common pattern of backbone chiral centers have a common pattern of backbone phosphorus modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have a common pattern of backbone phosphorus modifications and a common pattern of sugar modifications. In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers are identical.

In some embodiments, oligonucleotides in provided compositions have a common pattern of backbone phosphorus modifications. In some embodiments, a common base sequence is a base sequence of an oligonucleotide type.

As noted above and understood in the art, in some embodiments, base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, a particular oligonucleotide type may be defined by
1A) base identity;
1B) pattern of base modification;
1C) pattern of sugar modification;
2) pattern of backbone linkages;
3) pattern of backbone chiral centers; and
4) pattern of backbone phosphorus modifications.
   Thus, in some embodiments, oligonucleotides of a particular type may share identical bases but differ in their pattern of base modifications and/or sugar modifications. In some embodiments, oligonucleotides of a particular type may share identical bases and pattern of base modifications (including, e.g., absence of base modification), but differ in pattern of sugar modifications.

In some embodiments, oligonucleotides of a particular type are identical in that they have the same base sequence (including length), the same pattern of chemical modifications to sugar and base moieties, the same pattern of backbone linkages (e.g., pattern of natural phosphate linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as —S—, and -L-R¹ of formula I).

In some embodiments, purity of a chirally controlled oligonucleotide composition of an oligonucleotide type is expressed as the percentage of oligonucleotides in the composition that are of the oligonucleotide type. In some embodiments, at least about 10% of the oligonucleotides in a chirally controlled oligonucleotide composition are of the same oligonucleotide type. In some embodiments, at least about 20% of the oligonucleotides in a chirally controlled oligonucleotide composition are of the same oligonucleotide type. In some embodiments, at least about 30% of the oligonucleotides in a chirally controlled oligonucleotide composition are of the same oligonucleotide type. In some embodiments, at least about 40% of the oligonucleotides in a chirally controlled oligonucleotide composition are of the same oligonucleotide type. In some embodiments, at least about 50% of the oligonucleotides in a chirally controlled oligonucleotide composition are of the same oligonucleotide type. In some embodiments, at least about 60% of the oligonucleotides in a chirally controlled oligonucleotide composition are of the same oligonucleotide type. In some embodiments, at least about 70% of the oligonucleotides in a chirally controlled oligonucleotide composition are of the same oligonucleotide type. In some embodiments, at least about 80% of the oligonucleotides in a chirally controlled oligonucleotide composition are of the same oligonucleotide type. In some embodiments, at least about 90% of the oligonucleotides in a chirally controlled oligonucleotide composition are of the same oligonucleotide type. In some embodiments, at least about 92% of the oligonucleotides in a chirally controlled oligonucleotide composition are of the same oligonucleotide type. In some embodiments, at least about 94% of the oligonucleotides in a chirally controlled oligonucleotide composition are of the same oligonucleotide type. In some embodiments, at least about 95% of the oligonucleotides in a chirally controlled oligonucleotide composition are of the same oligonucleotide type. In some embodiments, at least about 96% of the oligonucleotides in a chirally controlled oligonucleotide composition are of the same oligonucleotide type. In some embodiments, at least about 97% of the oligonucleotides in a chirally controlled oligonucleotide composition are of the same oligonucleotide type. In some embodiments, at least about 98% of the oligonucleotides in a chirally controlled oligonucleotide composition are of the same oligonucleotide type. In some embodiments, at least about 99% of the oligonucleotides in a chirally controlled oligonucleotide composition are of the same oligonucleotide type.

In some embodiments, purity of a chirally controlled oligonucleotide composition can be controlled by stereoselectivity of each coupling step in its preparation process. In some embodiments, a coupling step has a stereoselectivity (e.g., diastereoselectivity) of 60% (60% of the new internucleotidic linkage formed from the coupling step has the intended stereochemistry). After such a coupling step, the new internucleotidic linkage formed may be referred to have a 60% purity. In some embodiments, each coupling step has a stereoselectivity of at least 60%. In some embodiments, each coupling step has a stereoselectivity of at least 70%. In some embodiments, each coupling step has a stereoselectivity of at least 80%. In some embodiments, each coupling step has a stereoselectivity of at least 85%. In some embodiments, each coupling step has a stereoselectivity of at least 90%. In some embodiments, each coupling step has a stereoselectivity of at least 91%. In some embodiments, each coupling step has a stereoselectivity of at least 92%. In some embodiments, each coupling step has a stereoselectivity of at least 93%. In some embodiments, each coupling step has a stereoselectivity of at least 94%. In some embodiments, each coupling step has a stereoselectivity of at least 95%. In some embodiments, each coupling step has a stereoselectivity of at least 96%. In some embodiments, each coupling step has a stereoselectivity of at least 97%. In some embodiments, each coupling step has a stereoselectivity of at least 98%. In some embodiments, each coupling step has a stereoselectivity of at least 99%. In some embodiments, each coupling step has a stereoselectivity of at least 99.5%. In some embodiments, each coupling step has a stereoselectivity of virtually 100%. In some embodiments, a coupling step has a stereoselectivity of virtually 100% in that all detectable product from the coupling step by an analytical method (e.g., NMR, HPLC, etc) has the intended stereoselectivity. In some embodiments, stereoselectivity of a chiral internucleotidic linkage in an oligonucleotide may be measured through a model reaction, e.g. formation of a dimer under essentially the same or comparable conditions wherein the dimer has the same internucleotidic linkage as the chiral internucleotidic linkage, the 5′-nucleoside of the dimer is the same as the nucleoside to the 5′-end of the chiral internucleotidic linkage, and the 3′-nucleoside of the dimer is the same as the nucleoside to the 3′-end of the chiral internucleotidic linkage (e.g., for fU*SfU*SfC*SfU, through the dimer of fU*SfC). As appreciated by a person having ordinary skill in the art, percentage of oligonucleotides of a particular type having n internucleotidic linkages in a preparation may be calculated as SE¹*SE²*SE³* . . . SEⁿ, wherein SE¹, SE², SE³, . . . , SEⁿ is independently the stereoselectivity of the 1^{st}, 2^{nd}, 3^{rd}, . . . , and n^{th} chiral internucleotidic linkage.

In some embodiments, in provided compositions, at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 97% or 99% of oligonucleotides that have the base sequence of a particular oligonucleotide type (defined by 1) base sequence; 2) pattern of backbone linkages; 3) pattern of backbone chiral centers; and 4) pattern of backbone phosphorus modifications) are oligonucleotides of the particular oligonucleotide type. In some embodiments, at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 97% or 99% of oligonucleotides that have the base sequence, the pattern of backbone linkages, and the pattern of backbone phosphorus modifications of a particular oligonucleotide type are oligonucleotides of the particular oligonucleotide type. In some embodiments, the percentage is at least 0.5%. In some embodiments, the percentage is at least 1%. In some embodiments, the percentage is at least 2%. In some embodiments, the percentage is at least 3%. In some embodiments, the percentage is at least 4%. In some embodiments, the percentage is at least 5%. In some embodiments, the percentage is at least 6%. In some embodiments, the percentage is at least 7%. In some embodiments, the percentage is at least 8%. In some embodiments, the percentage is at least 9%. In some embodiments, the percentage is at least 10%. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 70%. In some embodiments, the percentage is at least 75%. In some embodiments, the percentage is at least 80%. In some embodiments, the percentage is at least 81%. In some embodiments, the percentage is at least 82%. In some embodiments, the percentage is at least 83%. In some embodiments, the percentage is at least 84%. In some embodiments, the percentage is at least 85%. In some embodiments, the percentage is at least 86%. In some embodiments, the percentage is at least 87%. In some embodiments, the percentage is at least 88%. In some embodiments, the percentage is at least 89%. In some embodiments, the percentage is at least 90%. In some embodiments, the percentage is at least 91%. In some embodiments, the percentage is at least 92%. In some embodiments, the percentage is at least 93%. In some embodiments, the percentage is at least 94%. In some embodiments, the percentage is at least 95%. In some embodiments, the percentage is at least 96%. In some embodiments, the percentage is at least 97%. In some embodiments, the percentage is at least 98%. In some embodiments, the percentage is at least 99%.

As described herein, in some embodiments, provided oligonucleotides comprises one or more wing regions and a core region. In some embodiments, a wing region comprises a structural feature that is not in a core region. In some embodiments, a wing and core can be defined by any structural elements, e.g., base modifications (e.g., methylated/non-methylated, methylation at position 1/methylation at position 2, etc.), sugar modifications (e.g., modified/non-modified, 2′-modification/another type of modification, one type of 2′-modification/another type of 2′-modification, etc.), backbone linkage types (e.g., phosphate/phosphorothioate, phosphorothioate/substituted phosphorothioate, etc.), backbone chiral center stereochemistry (e.g., all Sp/all Rp, (SpRp) repeats/all Rp, etc.), backbone phosphorus modification types (e.g., s1/s2, s1/s3, etc.), etc.

In some embodiments, a wing and core is defined by nucleoside modifications, wherein a wing comprises a nucleoside modification that the core region does not have. In some embodiments, a wing and core is defined by sugar modifications, wherein a wing comprises a sugar modification that the core region does not have. In some embodiments, a sugar modification is a 2′-modification. In some embodiments, a sugar modification is 2′-OR¹. In some embodiments, a sugar modification is 2′-MOE. In some embodiments, a sugar modification is 2′-OMe. Additionally example sugar modifications are described in the present disclosure. In some embodiments, a wing and core is defined by internucleotidic linkages, wherein a wing comprises a internucleotidic linkage type (e.g., natural phosphate linkage, a type of modified internucleotidic linkage, etc.) that the core region does not have. In some embodiments, a wing and core is defined by internucleotidic linkages, wherein a wing has a pattern of backbone linkage that is different from that of the core.

In some embodiments, oligonucleotides in provided compositions have a wing-core or core-wing structure (hemimer). In some embodiments, oligonucleotides in provided compositions have a wing-core structure of nucleoside modifications. In some embodiments, oligonucleotides in provided compositions have a core-wing structure (another type of hemimer). In some embodiments, oligonucleotides in provided compositions have a core-wing structure of nucleoside modifications. In some embodiments, oligonucleotides in provided compositions have a wing-core-wing structure (gapmer). In some embodiments, oligonucleotides in provided compositions have a wing-core-wing structure of nucleoside modifications. In some embodiments, a wing and core is defined by modifications of the sugar moieties. In some embodiments, a wing and core is defined by modifications of the base moieties. In some embodiments, each sugar moiety in the wing region has the same 2′-modification which is not found in the core region. In some embodiments, each sugar moiety in the wing region has the same 2′-modification which is different than any sugar modifications in the core region. In some embodiments, a core region has no sugar modification. In some embodiments, each sugar moiety in the wing region has the same 2′-modification, and the core region has no 2′-modifications. In some embodiments, when two or more wings are present, each wing is defined by its own modifications. In some embodiments, each wing has its own characteristic sugar modification. In some embodiments, each wing has the same characteristic sugar modification differentiating it from a core. In some embodiments, each wing sugar moiety has the same modification. In some embodiments, each wing sugar moiety has the same 2′-modification. In some embodiments, each sugar moiety in a wing region has the same 2′-modification, yet the common 2′-modification in a first wing region can either be the same as or different from the common 2′-modification in a second wing region. In some embodiments, each sugar moiety in a wing region has the same 2′-modification, and the common 2′-modification in a first wing region is the same as the common 2′-modification in a second wing region. In some embodiments, each sugar moiety in a wing region has the same 2′-modification, and the common 2′-modification in a first wing region is different from the common 2′-modification in a second wing region.

In some embodiments, provided chirally controlled (and/or stereochemically pure) preparations are antisense oligonucleotides (e.g., chiromersen). In some embodiments, provided chirally controlled (and/or stereochemically pure) preparations are siRNA oligonucleotides. In some embodiments, a provided chirally controlled oligonucleotide composition is of oligonucleotides that can be antisense oligonucleotide, antagomir, microRNA, pre-microRNs, antimir, supermir, ribozyme, Ul adaptor, RNA activator, RNAi agent, decoy oligonucleotide, triplex forming oligonucleotide, aptamer or adjuvant. In some embodiments, a chirally controlled oligonucleotide composition is of antisense oligonucleotides. In some embodiments, a chirally controlled oligonucleotide composition is of antagomir oligonucleotides. In some embodiments, a chirally controlled oligonucleotide composition is of microRNA oligonucleotides. In some embodiments, a chirally controlled oligonucleotide composition is of pre-microRNA oligonucleotides. In some embodiments, a chirally controlled oligonucleotide composition is of antimir oligonucleotides. In some embodiments, a chirally controlled oligonucleotide composition is of supermir oligonucleotides. In some embodiments, a chirally controlled oligonucleotide composition is of ribozyme oligonucleotides. In some embodiments, a chirally controlled oligonucleotide composition is of Ul adaptor oligonucleotides. In some embodiments, a chirally controlled oligonucleotide composition is of RNA activator oligonucleotides. In some embodiments, a chirally controlled oligonucleotide composition is of RNAi agent oligonucleotides. In some embodiments, a chirally controlled oligonucleotide composition is of decoy oligonucleotides. In some embodiments, a chirally controlled oligonucleotide composition is of triplex forming oligonucleotides. In some embodiments, a chirally controlled oligonucleotide composition is of aptamer oligonucleotides. In some embodiments, a chirally controlled oligonucleotide composition is of adjuvant oligonucleotides.

In some embodiments, provided chirally controlled (and/or stereochemically pure) preparations are of oligonucleotides that include one or more modified backbone linkages, bases, and/or sugars.

In some embodiments, a provided oligonucleotide comprises one or more chiral, modified phosphate linkages. In some embodiments, a provided oligonucleotide comprises two or more chiral, modified phosphate linkages. In some embodiments, a provided oligonucleotide comprises three or more chiral, modified phosphate linkages. In some embodiments, a provided oligonucleotide comprises four or more chiral, modified phosphate linkages. In some embodiments, a provided oligonucleotide comprises five or more chiral, modified phosphate linkages. In some embodiments, a provided oligonucleotide comprises 1, 2, 3, 4, 5, 6, 7, 8, 9, 10, 11, 12, 13, 14, 15, 16, 17, 18, 19, 20, 21, 22, 23, 24, or 25 chiral, modified phosphate linkages. In some embodiments, a provided oligonucleotide type comprises 5 or more chiral, modified phosphate linkages. In some embodiments, a provided oligonucleotide type comprises 6 or more chiral, modified phosphate linkages. In some embodiments, a provided oligonucleotide type comprises 7 or more chiral, modified phosphate linkages. In some embodiments, a provided oligonucleotide type comprises 8 or more chiral, modified phosphate linkages. In some embodiments, a provided oligonucleotide type comprises 9 or more chiral, modified phosphate linkages. In some embodiments, a provided oligonucleotide type comprises 10 or more chiral, modified phosphate linkages. In some embodiments, a provided oligonucleotide type comprises 11 or more chiral, modified phosphate linkages. In some embodiments, a provided oligonucleotide type comprises 12 or more chiral, modified phosphate linkages. In some embodiments, a provided oligonucleotide type comprises 13 or more chiral, modified phosphate linkages. In some embodiments, a provided oligonucleotide type comprises 14 or more chiral, modified phosphate linkages. In some embodiments, a provided oligonucleotide type comprises 15 or more chiral, modified phosphate linkages. In some embodiments, a provided oligonucleotide type comprises 16 or more chiral, modified phosphate linkages. In some embodiments, a provided oligonucleotide type comprises 17 or more chiral, modified phosphate linkages. In some embodiments, a provided oligonucleotide type comprises 18 or more chiral, modified phosphate linkages. In some embodiments, a provided oligonucleotide type comprises 19 or more chiral, modified phosphate linkages. In some embodiments, a provided oligonucleotide type comprises 20 or more chiral, modified phosphate linkages. In some embodiments, a provided oligonucleotide type comprises 21 or more chiral, modified phosphate linkages. In some embodiments, a provided oligonucleotide type comprises 22 or more chiral, modified phosphate linkages. In some embodiments, a provided oligonucleotide type comprises 23 or more chiral, modified phosphate linkages. In some embodiments, a provided oligonucleotide type comprises 24 or more chiral, modified phosphate linkages. In some embodiments, a provided oligonucleotide type comprises 25 or more chiral, modified phosphate linkages.

In some embodiments, a provided oligonucleotide comprises at least 5%, 10%, 15%, 20%, 25%, 30%, 35%, 40%, 45%, 50%, 55%, 60%, 65%, 70%, 75%, 80%, 85%, 90%, 95%, or 100% chiral, modified phosphate linkages. Example such chiral, modified phosphate linkages are described above and herein. In some embodiments, a provided oligonucleotide comprises at least 5%, 10%, 15%, 20%, 25%, 30%, 35%, 40%, 45%, 50%, 55%, 60%, 65%, 70%, 75%, 80%, 85%, 90%, 95%, or 100% chiral, modified phosphate linkages in the Sp configuration.

In some embodiments, provided chirally controlled (and/or stereochemically pure) preparations are of a stereochemical purity of greater than about 80%. In some embodiments, provided chirally controlled (and/or stereochemically pure) preparations are of a stereochemical purity of greater than about 85%. In some embodiments, provided chirally controlled (and/or stereochemically pure) preparations are of a stereochemical purity of greater than about 90%. In some embodiments, provided chirally controlled (and/or stereochemically pure) preparations are of a stereochemical purity of greater than about 91%. In some embodiments, provided chirally controlled (and/or stereochemically pure) preparations are of a stereochemical purity of greater than about 92%. In some embodiments, provided chirally controlled (and/or stereochemically pure) preparations are of a stereochemical purity of greater than about 93%. In some embodiments, provided chirally controlled (and/or stereochemically pure) preparations are of a stereochemical purity of greater than about 94%. In some embodiments, provided chirally controlled (and/or stereochemically pure) preparations are of a stereochemical purity of greater than about 95%. In some embodiments, provided chirally controlled (and/or stereochemically pure) preparations are of a stereochemical purity of greater than about 96%. In some embodiments, provided chirally controlled (and/or stereochemically pure) preparations are of a stereochemical purity of greater than about 97%. In some embodiments, provided chirally controlled (and/or stereochemically pure) preparations are of a stereochemical purity of greater than about 98%. In some embodiments, provided chirally controlled (and/or stereochemically pure) preparations are of a stereochemical purity of greater than about 99%. In some embodiments, such a provided purity can be of one or more chiral internucleotidic linkage is a composition is partially chirally controlled.

In some embodiments, a chiral, modified phosphate linkage is a chiral phosphorothioate linkage, i.e., phosphorothioate internucleotidic linkage. In some embodiments, a provided oligonucleotide comprises at least 5%, 10%, 15%, 20%, 25%, 30%, 35%, 40%, 45%, 50%, 55%, 60%, 65%, 70%, 75%, 80%, 85%, 90%, 95%, or 100% chiral phosphorothioate internucleotidic linkages. In some embodiments, all chiral, modified phosphate linkages are chiral phosphorothioate internucleotidic linkages. In some embodiments, at least about 10, 20, 30, 40, 50, 60, 70, 80, or 90% chiral phosphorothioate internucleotidic linkages of a provided oligonucleotide are of the Sp conformation. In some embodiments, at least about 10% chiral phosphorothioate internucleotidic linkages of a provided oligonucleotide are of the Sp conformation. In some embodiments, at least about 20% chiral phosphorothioate internucleotidic linkages of a provided oligonucleotide are of the Sp conformation. In some embodiments, at least about 30% chiral phosphorothioate internucleotidic linkages of a provided oligonucleotide are of the Sp conformation. In some embodiments, at least about 40% chiral phosphorothioate internucleotidic linkages of a provided oligonucleotide are of the Sp conformation. In some embodiments, at least about 50% chiral phosphorothioate internucleotidic linkages of a provided oligonucleotide are of the Sp conformation. In some embodiments, at least about 60% chiral phosphorothioate internucleotidic linkages of a provided oligonucleotide are of the Sp conformation. In some embodiments, at least about 70% chiral phosphorothioate internucleotidic linkages of a provided oligonucleotide are of the Sp conformation. In some embodiments, at least about 80% chiral phosphorothioate internucleotidic linkages of a provided oligonucleotide are of the Sp conformation. In some embodiments, at least about 90% chiral phosphorothioate internucleotidic linkages of a provided oligonucleotide are of the Sp conformation. In some embodiments, at least about 95% chiral phosphorothioate internucleotidic linkages of a provided oligonucleotide are of the Sp conformation.

In some embodiments, at least about 10, 20, 30, 40, 50, 60, 70, 80, or 90% chiral phosphorothioate internucleotidic linkages of a provided oligonucleotide are of the Rp conformation. In some embodiments, at least about 10% chiral phosphorothioate internucleotidic linkages of a provided oligonucleotide are of the Rp conformation. In some embodiments, at least about 20% chiral phosphorothioate internucleotidic linkages of a provided oligonucleotide are of the Rp conformation. In some embodiments, at least about 30% chiral phosphorothioate internucleotidic linkages of a provided oligonucleotide are of the Rp conformation. In some embodiments, at least about 40% chiral phosphorothioate internucleotidic linkages of a provided oligonucleotide are of the Rp conformation. In some embodiments, at least about 50% chiral phosphorothioate internucleotidic linkages of a provided oligonucleotide are of the Rp conformation. In some embodiments, at least about 60% chiral phosphorothioate internucleotidic linkages of a provided oligonucleotide are of the Rp conformation. In some embodiments, at least about 70% chiral phosphorothioate internucleotidic linkages of a provided oligonucleotide are of the Rp conformation. In some embodiments, at least about 80% chiral phosphorothioate internucleotidic linkages of a provided oligonucleotide are of the Rp conformation. In some embodiments, at least about 90% chiral phosphorothioate internucleotidic linkages of a provided oligonucleotide are of the Rp conformation. In some embodiments, at least about 95% chiral phosphorothioate internucleotidic linkages of a provided oligonucleotide are of the Rp conformation.

In some embodiments, less than about 10, 20, 30, 40, 50, 60, 70, 80, or 90% chiral phosphorothioate internucleotidic linkages of a provided oligonucleotide are of the Rp conformation. In some embodiments, less than about 10% chiral phosphorothioate internucleotidic linkages of a provided oligonucleotide are of the Rp conformation. In some embodiments, less than about 20% chiral phosphorothioate internucleotidic linkages of a provided oligonucleotide are of the Rp conformation. In some embodiments, less than about 30% chiral phosphorothioate internucleotidic linkages of a provided oligonucleotide are of the Rp conformation. In some embodiments, less than about 40% chiral phosphorothioate internucleotidic linkages of a provided oligonucleotide are of the Rp conformation. In some embodiments, less than about 50% chiral phosphorothioate internucleotidic linkages of a provided oligonucleotide are of the Rp conformation. In some embodiments, less than about 60% chiral phosphorothioate internucleotidic linkages of a provided oligonucleotide are of the Rp conformation. In some embodiments, less than about 70% chiral phosphorothioate internucleotidic linkages of a provided oligonucleotide are of the Rp conformation. In some embodiments, less than about 80% chiral phosphorothioate internucleotidic linkages of a provided oligonucleotide are of the Rp conformation. In some embodiments, less than about 90% chiral phosphorothioate internucleotidic linkages of a provided oligonucleotide are of the Rp conformation. In some embodiments, less than about 95% chiral phosphorothioate internucleotidic linkages of a provided oligonucleotide are of the Rp conformation. In some embodiments, a provided oligonucleotide has only one Rp chiral phosphorothioate internucleotidic linkages. In some embodiments, a provided oligonucleotide has only one Rp chiral phosphorothioate internucleotidic linkages, wherein all internucleotide linkages are chiral phosphorothioate internucleotidic linkages.

In some embodiments, a chiral phosphorothioate internucleotidic linkage is a chiral phosphorothioate diester linkage. In some embodiments, each chiral phosphorothioate internucleotidic linkage is independently a chiral phosphorothioate diester linkage. In some embodiments, each internucleotidic linkage is independently a chiral phosphorothioate diester linkage. In some embodiments, each internucleotidic linkage is independently a chiral phosphorothioate diester linkage, and only one is Rp.

In some embodiments, provided chirally controlled (and/or stereochemically pure) preparations are of oligonucleotides that contain one or more modified bases. In some embodiments, provided chirally controlled (and/or stereochemically pure) preparations are of oligonucleotides that contain no modified bases. Example such modified bases are described above and herein.

In some embodiments, an oligonucleotide comprises at least one internucleotidic linkage which is chirally controlled (e.g., a phosphorothioate in Sp or Rp configuration) and at least one internucleotidic linkage which is not chiral (e.g., a phosphodiester or phosphorodithioate). In some embodiments, an oligonucleotide comprises at least one internucleotidic linkage which is chirally controlled phosphorothioate in Sp configuration and at least one internucleotidic linkage which is not chiral (e.g., a phosphodiester or phosphorodithioate).

In some embodiments, oligonucleotides of provided compositions comprise at least 2, 3, 4, 5, 6, 7, 8, 9 or 10 natural phosphate linkages. In some embodiments, oligonucleotides of provided compositions comprise at least one natural phosphate linkage. In some embodiments, oligonucleotides of provided compositions comprise at least two natural phosphate linkages. In some embodiments, oligonucleotides of provided compositions comprise at least three natural phosphate linkages. In some embodiments, oligonucleotides of provided compositions comprise at least four natural phosphate linkages. In some embodiments, oligonucleotides of provided compositions comprise at least five natural phosphate linkages. In some embodiments, oligonucleotides of provided compositions comprise at least six natural phosphate linkages. In some embodiments, oligonucleotides of provided compositions comprise at least seven natural phosphate linkages. In some embodiments, oligonucleotides of provided compositions comprise at least eight natural phosphate linkages. In some embodiments, oligonucleotides of provided compositions comprise at least nine natural phosphate linkages. In some embodiments, oligonucleotides of provided compositions comprise at least ten natural phosphate linkages.

In some embodiments, oligonucleotides of provided compositions comprise 2, 3, 4, 5, 6, 7, 8, 9 or 10 natural phosphate linkages. In some embodiments, oligonucleotides of provided compositions comprise one natural phosphate linkage. In some embodiments, oligonucleotides of provided compositions comprise two natural phosphate linkages. In some embodiments, oligonucleotides of provided compositions comprise three natural phosphate linkages. In some embodiments, oligonucleotides of provided compositions comprise four natural phosphate linkages. In some embodiments, oligonucleotides of provided compositions comprise five natural phosphate linkages. In some embodiments, oligonucleotides of provided compositions comprise six natural phosphate linkages. In some embodiments, oligonucleotides of provided compositions comprise seven natural phosphate linkages. In some embodiments, oligonucleotides of provided compositions comprise eight natural phosphate linkages. In some embodiments, oligonucleotides of provided compositions comprise nine natural phosphate linkages. In some embodiments, oligonucleotides of provided compositions comprise ten natural phosphate linkages.

In some embodiments, oligonucleotides of provided compositions comprise at least 2, 3, 4, 5, 6, 7, 8, 9 or 10 consecutive natural phosphate linkages. In some embodiments, oligonucleotides of provided compositions comprise at least two consecutive natural phosphate linkages. In some embodiments, oligonucleotides of provided compositions comprise at least three consecutive natural phosphate linkages. In some embodiments, oligonucleotides of provided compositions comprise at least four consecutive natural phosphate linkages. In some embodiments, oligonucleotides of provided compositions comprise at least five consecutive natural phosphate linkages. In some embodiments, oligonucleotides of provided compositions comprise at least six consecutive natural phosphate linkages. In some embodiments, oligonucleotides of provided compositions comprise at least seven consecutive natural phosphate linkages. In some embodiments, oligonucleotides of provided compositions comprise at least eight consecutive natural phosphate linkages. In some embodiments, oligonucleotides of provided compositions comprise at least nine consecutive natural phosphate linkages. In some embodiments, oligonucleotides of provided compositions comprise at least ten consecutive natural phosphate linkages.

In some embodiments, oligonucleotides of provided compositions comprise 2, 3, 4, 5, 6, 7, 8, 9 or 10 consecutive natural phosphate linkages. In some embodiments, oligonucleotides of provided compositions comprise two consecutive natural phosphate linkages. In some embodiments, oligonucleotides of provided compositions comprise three consecutive natural phosphate linkages. In some embodiments, oligonucleotides of provided compositions comprise four consecutive natural phosphate linkages. In some embodiments, oligonucleotides of provided compositions comprise five consecutive natural phosphate linkages. In some embodiments, oligonucleotides of provided compositions comprise six consecutive natural phosphate linkages. In some embodiments, oligonucleotides of provided compositions comprise seven consecutive natural phosphate linkages. In some embodiments, oligonucleotides of provided compositions comprise eight consecutive natural phosphate linkages. In some embodiments, oligonucleotides of provided compositions comprise nine consecutive natural phosphate linkages. In some embodiments, oligonucleotides of provided compositions comprise ten consecutive natural phosphate linkages.

In some embodiments, provided chirally controlled (and/or stereochemically pure) preparations are of oligonucleotides having a common base sequence of at least 8 bases. In some embodiments, provided chirally controlled (and/or stereochemically pure) preparations are of oligonucleotides having a common base sequence of at least 9 bases. In some embodiments, provided chirally controlled (and/or stereochemically pure) preparations are of oligonucleotides having a common base sequence of at least 10 bases. In some embodiments, provided chirally controlled (and/or stereochemically pure) preparations are of oligonucleotides having a common base sequence of at least 11 bases. In some embodiments, provided chirally controlled (and/or stereochemically pure) preparations are of oligonucleotides having a common base sequence of at least 12 bases. In some embodiments, provided chirally controlled (and/or stereochemically pure) preparations are of oligonucleotides having a common base sequence of at least 13 bases. In some embodiments, provided chirally controlled (and/or stereochemically pure) preparations are of oligonucleotides having a common base sequence of at least 14 bases. In some embodiments, provided chirally controlled (and/or stereochemically pure) preparations are of oligonucleotides having a common base sequence of at least 15 bases. In some embodiments, provided chirally controlled (and/or stereochemically pure) preparations are of oligonucleotides having a common base sequence of at least 16 bases. In some embodiments, provided chirally controlled (and/or stereochemically pure) preparations are of oligonucleotides having a common base sequence of at least 17 bases. In some embodiments, provided chirally controlled (and/or stereochemically pure) preparations are of oligonucleotides having a common base sequence of at least 18 bases. In some embodiments, provided chirally controlled (and/or stereochemically pure) preparations are of oligonucleotides having a common base sequence of at least 19 bases. In some embodiments, provided chirally controlled (and/or stereochemically pure) preparations are of oligonucleotides having a common base sequence of at least 20 bases. In some embodiments, provided chirally controlled (and/or stereochemically pure) preparations are of oligonucleotides having a common base sequence of at least 21 bases. In some embodiments, provided chirally controlled (and/or stereochemically pure) preparations are of oligonucleotides having a common base sequence of at least 22 bases. In some embodiments, provided chirally controlled (and/or stereochemically pure) preparations are of oligonucleotides having a common base sequence of at least 23 bases. In some embodiments, provided chirally controlled (and/or stereochemically pure) preparations are of oligonucleotides having a common base sequence of at least 24 bases. In some embodiments, provided chirally controlled (and/or stereochemically pure) preparations are of oligonucleotides having a common base sequence of at least 25 bases. In some embodiments, provided chirally controlled (and/or stereochemically pure) preparations are of oligonucleotides having a common base sequence of at least 30, 35, 40, 45, 50, 55, 60, 65, 70, or 75 bases.

In some embodiments, provided compositions comprise oligonucleotides containing one or more residues which are modified at the sugar moiety. In some embodiments, provided compositions comprise oligonucleotides containing one or more residues which are modified at the 2′ position of the sugar moiety (referred to herein as a “2′-modification”). Example such modifications are described above and herein and include, but are not limited to, 2′-OMe, 2′-MOE, 2′-LNA, 2′-F, FRNA, FANA, S-cEt, etc. In some embodiments, provided compositions comprise oligonucleotides containing one or more residues which are 2′-modified. For example, in some embodiments, provided oligonucleotides contain one or more residues which are 2′-O-methoxyethyl (2′-MOE)-modified residues. In some embodiments, provided compositions comprise oligonucleotides which do not contain any 2′-modifications. In some embodiments, provided compositions are oligonucleotides which do not contain any 2′-MOE residues. That is, in some embodiments, provided oligonucleotides are not MOE-modified. Additional example sugar modifications are described in the present disclosure.

In some embodiments, provided oligonucleotides are of a general motif of wing-core or core-wing (hemimer, also represented herein generally as X—Y or Y—X, respectively). In some embodiments, provided oligonucleotides are of a general motif of wing-core-wing (gapmer, also represented herein generally as X—Y—X). In some embodiments, each wing region independently contains one or more residues having a particular modification, which modification is absent from the core “Y” portion. In some embodiments, each wing region independently contains one or more residues having a particular nucleoside modification, which modification is absent from the core “Y” portion. In some embodiments, each wing region independently contains one or more residues having a particular base modification, which modification is absent from the core “Y” portion. In some embodiments, each wing region independently contains one or more residues having a particular sugar modification, which modification is absent from the core “Y” portion. Example sugar modifications are widely known in the art. In some embodiments, a sugar modification is a modification selected from those modifications described in U.S. Pat. No. 9,006,198, which sugar modifications are incorporated herein by references. Additional example sugar modifications are described in the present disclosure. In some embodiment, each wing contains one or more residues having a 2′ modification that is not present in the core portion. In some embodiments, a 2′-modification is 2′-OR¹, wherein R¹ is as defined and described in the present disclosure.

In some embodiments, provided oligonucleotides have a wing-core motif represented as X—Y, or a core-wing motif represented as Y—X, wherein the residues at the “X” portion are sugar modified residues of a particular type and the residues in the core “Y” portion are not sugar modified residues of the same particular type. In some embodiments, provided oligonucleotides have a wing-core-wing motif represented as X—Y—X, wherein the residues at each “X” portion are sugar modified residues of a particular type and the residues in the core “Y” portion are not sugar modified residues of the same particular type. In some embodiments, provided oligonucleotides have a wing-core motif represented as X—Y, or a core-wing motif represented as Y—X, wherein the residues at the “X” portion are 2′-modified residues of a particular type and the residues in the core “Y” portion are not 2′-modified residues of the same particular type. In some embodiments, provided oligonucleotides have a wing-core motif represented as X—Y, wherein the residues at the “X” portion are 2′-modified residues of a particular type and the residues in the core “Y” portion are not 2′-modified residues of the same particular type. In some embodiments, provided oligonucleotides have a core-wing motif represented as Y—X, wherein the residues at the “X” portion are 2′-modified residues of a particular type and the residues in the core “Y” portion are not 2′-modified residues of the same particular type. In some embodiments, provided oligonucleotides have a wing-core-wing motif represented as X—Y—X, wherein the residues at each “X” portion are 2′-modified residues of a particular type and the residues in the core “Y” portion are not 2′-modified residues of the same particular type. In some embodiments, provided oligonucleotides have a wing-core motif represented as X—Y, wherein the residues at the “X” portion are 2′-modified residues of a particular type and the residues in the core “Y” portion are 2′-deoxyribonucleoside. In some embodiments, provided oligonucleotides have a core-wing motif represented as Y—X, wherein the residues at the “X” portion are 2′-modified residues of a particular type and the residues in the core “Y” portion are 2′-deoxyribonucleoside. In some embodiments, provided oligonucleotides have a wing-core-wing motif represented as X—Y—X, wherein the residues at each “X” portion are 2′-modified residues of a particular type and the residues in the core “Y” portion are 2′-deoxyribonucleoside. In some embodiments, provided oligonucleotides have a wing-core-wing motif represented as X—Y—X, wherein the residues at each “X” portion are 2′-modified residues of a particular type and the residues in the core “Y” portion are 2′-deoxyribonucleoside. For instance, in some embodiments, provided oligonucleotides have a wing-core-wing motif represented as X—Y—X, wherein the residues at each “X” portion are 2′-MOE-modified residues and the residues in the core “Y” portion are not 2′-MOE-modified residues. In some embodiments, provided oligonucleotides have a wing-core-wing motif represented as X—Y—X, wherein the residues at each “X” portion are 2′-MOE-modified residues and the residues in the core “Y” portion are 2′-deoxyribonucleoside. One of skill in the relevant arts will recognize that all such 2′-modifications described above and herein are contemplated in the context of such X—Y, Y—X and/or X—Y—X motifs.

In some embodiments, a wing has a length of one or more bases. In some embodiments, a wing has a length of two or more bases. In some embodiments, a wing has a length of three or more bases. In some embodiments, a wing has a length of four or more bases. In some embodiments, a wing has a length of five or more bases. In some embodiments, a wing has a length of six or more bases. In some embodiments, a wing has a length of seven or more bases. In some embodiments, a wing has a length of eight or more bases. In some embodiments, a wing has a length of nine or more bases. In some embodiments, a wing has a length of ten or more bases. In some embodiments, a wing has a length of 11 or more bases. In some embodiments, a wing has a length of 12 or more bases. In some embodiments, a wing has a length of 13 or more bases. In some embodiments, a wing has a length of 14 or more bases. In some embodiments, a wing has a length of 15 or more bases. In some embodiments, a wing has a length of 16 or more bases. In some embodiments, a wing has a length of 17 or more bases. In some embodiments, a wing has a length of 18 or more bases. In some embodiments, a wing has a length of 19 or more bases. In some embodiments, a wing has a length of ten or more bases.

In some embodiments, a wing has a length of one base. In some embodiments, a wing has a length of two bases. In some embodiments, a wing has a length of three bases. In some embodiments, a wing has a length of four bases. In some embodiments, a wing has a length of five bases. In some embodiments, a wing has a length of six bases. In some embodiments, a wing has a length of seven bases. In some embodiments, a wing has a length of eight bases. In some embodiments, a wing has a length of nine bases. In some embodiments, a wing has a length of ten bases. In some embodiments, a wing has a length of 11 bases. In some embodiments, a wing has a length of 12 bases. In some embodiments, a wing has a length of 13 bases. In some embodiments, a wing has a length of 14 bases. In some embodiments, a wing has a length of 15 bases. In some embodiments, a wing has a length of 16 bases. In some embodiments, a wing has a length of 17 bases. In some embodiments, a wing has a length of 18 bases. In some embodiments, a wing has a length of 19 bases. In some embodiments, a wing has a length of ten bases.

In some embodiments, a wing comprises one or more chiral internucleotidic linkages. In some embodiments, a wing comprises one or more natural phosphate linkages. In some embodiments, a wing comprises one or more chiral internucleotidic linkages and one or more natural phosphate linkages. In some embodiments, a wing comprises one or more chiral internucleotidic linkages and two or more natural phosphate linkages. In some embodiments, a wing comprises one or more chiral internucleotidic linkages and two or more natural phosphate linkages, wherein two or more natural phosphate linkages are consecutive. In some embodiments, a wing comprises no chiral internucleotidic linkages. In some embodiments, each wing linkage is a natural phosphate linkage. In some embodiments, a wing comprises no phosphate linkages. In some embodiments, each wing is independently a chiral internucleotidic linkage.

In some embodiments, each wing region independently comprises one or more chiral internucleotidic linkages. In some embodiments, each wing region independently comprises one or more natural phosphate linkages. In some embodiments, each wing region independently comprises one or more chiral internucleotidic linkages and one or more natural phosphate linkages. In some embodiments, each wing region independently comprises one or more chiral internucleotidic linkages and two or more natural phosphate linkages. In some embodiments, each wing region independently comprises one or more chiral internucleotidic linkages and two or more natural phosphate linkages, wherein two or more natural phosphate linkages are consecutive.

In some embodiments, each wing region independently comprises at least one chiral internucleotidic linkage. In some embodiments, each wing region independently comprises at least two chiral internucleotidic linkages. In some embodiments, each wing region independently comprises at least three chiral internucleotidic linkages. In some embodiments, each wing region independently comprises at least four chiral internucleotidic linkages. In some embodiments, each wing region independently comprises at least five chiral internucleotidic linkages. In some embodiments, each wing region independently comprises at least six chiral internucleotidic linkages. In some embodiments, each wing region independently comprises at least seven chiral internucleotidic linkages. In some embodiments, each wing region independently comprises at least eight chiral internucleotidic linkages. In some embodiments, each wing region independently comprises at least nine chiral internucleotidic linkages. In some embodiments, each wing region independently comprises at least ten chiral internucleotidic linkages. In some embodiments, each wing region independently comprises at least 11 chiral internucleotidic linkages. In some embodiments, each wing region independently comprises at least 12 chiral internucleotidic linkages. In some embodiments, each wing region independently comprises at least 13 chiral internucleotidic linkages. In some embodiments, each wing region independently comprises at least 14 chiral internucleotidic linkages. In some embodiments, each wing region independently comprises at least 15 chiral internucleotidic linkages. In some embodiments, each wing region independently comprises at least 16 chiral internucleotidic linkages. In some embodiments, each wing region independently comprises at least 17 chiral internucleotidic linkages. In some embodiments, each wing region independently comprises at least 18 chiral internucleotidic linkages. In some embodiments, each wing region independently comprises at least 19 chiral internucleotidic linkages. In some embodiments, each wing region independently comprises at least 20 chiral internucleotidic linkages.

In some embodiments, each wing region independently comprises one chiral internucleotidic linkage. In some embodiments, each wing region independently comprises two chiral internucleotidic linkages. In some embodiments, each wing region independently comprises three chiral internucleotidic linkages. In some embodiments, each wing region independently comprises four chiral internucleotidic linkages. In some embodiments, each wing region independently comprises five chiral internucleotidic linkages. In some embodiments, each wing region independently comprises six chiral internucleotidic linkages. In some embodiments, each wing region independently comprises seven chiral internucleotidic linkages. In some embodiments, each wing region independently comprises eight chiral internucleotidic linkages. In some embodiments, each wing region independently comprises nine chiral internucleotidic linkages. In some embodiments, each wing region independently comprises ten chiral internucleotidic linkages. In some embodiments, each wing region independently comprises 11 chiral internucleotidic linkages. In some embodiments, each wing region independently comprises 12 chiral internucleotidic linkages. In some embodiments, each wing region independently comprises 13 chiral internucleotidic linkages. In some embodiments, each wing region independently comprises 14 chiral internucleotidic linkages. In some embodiments, each wing region independently comprises 15 chiral internucleotidic linkages. In some embodiments, each wing region independently comprises 16 chiral internucleotidic linkages. In some embodiments, each wing region independently comprises 17 chiral internucleotidic linkages. In some embodiments, each wing region independently comprises 18 chiral internucleotidic linkages. In some embodiments, each wing region independently comprises 19 chiral internucleotidic linkages. In some embodiments, each wing region independently comprises 20 chiral internucleotidic linkages.

In some embodiments, each wing region independently comprises at least one consecutive natural phosphate linkage. In some embodiments, each wing region independently comprises at least two consecutive chiral internucleotidic linkages. In some embodiments, each wing region independently comprises at least three consecutive chiral internucleotidic linkages. In some embodiments, each wing region independently comprises at least four consecutive chiral internucleotidic linkages. In some embodiments, each wing region independently comprises at least five consecutive chiral internucleotidic linkages. In some embodiments, each wing region independently comprises at least six consecutive chiral internucleotidic linkages. In some embodiments, each wing region independently comprises at least seven consecutive chiral internucleotidic linkages. In some embodiments, each wing region independently comprises at least eight consecutive chiral internucleotidic linkages. In some embodiments, each wing region independently comprises at least nine consecutive chiral internucleotidic linkages. In some embodiments, each wing region independently comprises at least ten consecutive chiral internucleotidic linkages. In some embodiments, each wing region independently comprises at least 11 consecutive chiral internucleotidic linkages. In some embodiments, each wing region independently comprises at least 12 consecutive chiral internucleotidic linkages. In some embodiments, each wing region independently comprises at least 13 consecutive chiral internucleotidic linkages. In some embodiments, each wing region independently comprises at least 14 consecutive chiral internucleotidic linkages. In some embodiments, each wing region independently comprises at least 15 consecutive chiral internucleotidic linkages. In some embodiments, each wing region independently comprises at least 16 consecutive chiral internucleotidic linkages. In some embodiments, each wing region independently comprises at least 17 consecutive chiral internucleotidic linkages. In some embodiments, each wing region independently comprises at least 18 consecutive chiral internucleotidic linkages. In some embodiments, each wing region independently comprises at least 19 consecutive chiral internucleotidic linkages. In some embodiments, each wing region independently comprises at least 20 consecutive chiral internucleotidic linkages.

In some embodiments, each wing region independently comprises one consecutive natural phosphate linkage. In some embodiments, each wing region independently comprises two consecutive chiral internucleotidic linkages. In some embodiments, each wing region independently comprises three consecutive chiral internucleotidic linkages. In some embodiments, each wing region independently comprises four consecutive chiral internucleotidic linkages. In some embodiments, each wing region independently comprises five consecutive chiral internucleotidic linkages. In some embodiments, each wing region independently comprises six consecutive chiral internucleotidic linkages. In some embodiments, each wing region independently comprises seven consecutive chiral internucleotidic linkages. In some embodiments, each wing region independently comprises eight consecutive chiral internucleotidic linkages. In some embodiments, each wing region independently comprises nine consecutive chiral internucleotidic linkages. In some embodiments, each wing region independently comprises ten consecutive chiral internucleotidic linkages. In some embodiments, each wing region independently comprises 11 consecutive chiral internucleotidic linkages. In some embodiments, each wing region independently comprises 12 consecutive chiral internucleotidic linkages. In some embodiments, each wing region independently comprises 13 consecutive chiral internucleotidic linkages. In some embodiments, each wing region independently comprises 14 consecutive chiral internucleotidic linkages. In some embodiments, each wing region independently comprises 15 consecutive chiral internucleotidic linkages. In some embodiments, each wing region independently comprises 16 consecutive chiral internucleotidic linkages. In some embodiments, each wing region independently comprises 17 consecutive chiral internucleotidic linkages. In some embodiments, each wing region independently comprises 18 consecutive chiral internucleotidic linkages. In some embodiments, each wing region independently comprises 19 consecutive chiral internucleotidic linkages. In some embodiments, each wing region independently comprises 20 consecutive chiral internucleotidic linkages.

In some embodiments, each wing region independently comprises at least one natural phosphate linkage. In some embodiments, each wing region independently comprises at least two natural phosphate linkages. In some embodiments, each wing region independently comprises at least three natural phosphate linkages. In some embodiments, each wing region independently comprises at least four natural phosphate linkages. In some embodiments, each wing region independently comprises at least five natural phosphate linkages. In some embodiments, each wing region independently comprises at least six natural phosphate linkages. In some embodiments, each wing region independently comprises at least seven natural phosphate linkages. In some embodiments, each wing region independently comprises at least eight natural phosphate linkages. In some embodiments, each wing region independently comprises at least nine natural phosphate linkages. In some embodiments, each wing region independently comprises at least ten natural phosphate linkages. In some embodiments, each wing region independently comprises at least 11 natural phosphate linkages. In some embodiments, each wing region independently comprises at least 12 natural phosphate linkages. In some embodiments, each wing region independently comprises at least 13 natural phosphate linkages. In some embodiments, each wing region independently comprises at least 14 natural phosphate linkages. In some embodiments, each wing region independently comprises at least 15 natural phosphate linkages. In some embodiments, each wing region independently comprises at least 16 natural phosphate linkages. In some embodiments, each wing region independently comprises at least 17 natural phosphate linkages. In some embodiments, each wing region independently comprises at least 18 natural phosphate linkages. In some embodiments, each wing region independently comprises at least 19 natural phosphate linkages. In some embodiments, each wing region independently comprises at least 20 natural phosphate linkages.

In some embodiments, each wing region independently comprises one natural phosphate linkage. In some embodiments, each wing region independently comprises two natural phosphate linkages. In some embodiments, each wing region independently comprises three natural phosphate linkages. In some embodiments, each wing region independently comprises four natural phosphate linkages. In some embodiments, each wing region independently comprises five natural phosphate linkages. In some embodiments, each wing region independently comprises six natural phosphate linkages. In some embodiments, each wing region independently comprises seven natural phosphate linkages. In some embodiments, each wing region independently comprises eight natural phosphate linkages. In some embodiments, each wing region independently comprises nine natural phosphate linkages. In some embodiments, each wing region independently comprises ten natural phosphate linkages. In some embodiments, each wing region independently comprises 11 natural phosphate linkages. In some embodiments, each wing region independently comprises 12 natural phosphate linkages. In some embodiments, each wing region independently comprises 13 natural phosphate linkages. In some embodiments, each wing region independently comprises 14 natural phosphate linkages. In some embodiments, each wing region independently comprises 15 natural phosphate linkages. In some embodiments, each wing region independently comprises 16 natural phosphate linkages. In some embodiments, each wing region independently comprises 17 natural phosphate linkages. In some embodiments, each wing region independently comprises 18 natural phosphate linkages. In some embodiments, each wing region independently comprises 19 natural phosphate linkages. In some embodiments, each wing region independently comprises 20 natural phosphate linkages.

In some embodiments, each wing region independently comprises at least one consecutive natural phosphate linkage. In some embodiments, each wing region independently comprises at least two consecutive natural phosphate linkages. In some embodiments, each wing region independently comprises at least three consecutive natural phosphate linkages. In some embodiments, each wing region independently comprises at least four consecutive natural phosphate linkages. In some embodiments, each wing region independently comprises at least five consecutive natural phosphate linkages. In some embodiments, each wing region independently comprises at least six consecutive natural phosphate linkages. In some embodiments, each wing region independently comprises at least seven consecutive natural phosphate linkages. In some embodiments, each wing region independently comprises at least eight consecutive natural phosphate linkages. In some embodiments, each wing region independently comprises at least nine consecutive natural phosphate linkages. In some embodiments, each wing region independently comprises at least ten consecutive natural phosphate linkages. In some embodiments, each wing region independently comprises at least 11 consecutive natural phosphate linkages. In some embodiments, each wing region independently comprises at least 12 consecutive natural phosphate linkages. In some embodiments, each wing region independently comprises at least 13 consecutive natural phosphate linkages. In some embodiments, each wing region independently comprises at least 14 consecutive natural phosphate linkages. In some embodiments, each wing region independently comprises at least 15 consecutive natural phosphate linkages. In some embodiments, each wing region independently comprises at least 16 consecutive natural phosphate linkages. In some embodiments, each wing region independently comprises at least 17 consecutive natural phosphate linkages. In some embodiments, each wing region independently comprises at least 18 consecutive natural phosphate linkages. In some embodiments, each wing region independently comprises at least 19 consecutive natural phosphate linkages. In some embodiments, each wing region independently comprises at least 20 consecutive natural phosphate linkages.

In some embodiments, each wing region independently comprises one consecutive natural phosphate linkage. In some embodiments, each wing region independently comprises two consecutive natural phosphate linkages. In some embodiments, each wing region independently comprises three consecutive natural phosphate linkages. In some embodiments, each wing region independently comprises four consecutive natural phosphate linkages. In some embodiments, each wing region independently comprises five consecutive natural phosphate linkages. In some embodiments, each wing region independently comprises six consecutive natural phosphate linkages. In some embodiments, each wing region independently comprises seven consecutive natural phosphate linkages. In some embodiments, each wing region independently comprises eight consecutive natural phosphate linkages. In some embodiments, each wing region independently comprises nine consecutive natural phosphate linkages. In some embodiments, each wing region independently comprises ten consecutive natural phosphate linkages. In some embodiments, each wing region independently comprises 11 consecutive natural phosphate linkages. In some embodiments, each wing region independently comprises 12 consecutive natural phosphate linkages. In some embodiments, each wing region independently comprises 13 consecutive natural phosphate linkages. In some embodiments, each wing region independently comprises 14 consecutive natural phosphate linkages. In some embodiments, each wing region independently comprises 15 consecutive natural phosphate linkages. In some embodiments, each wing region independently comprises 16 consecutive natural phosphate linkages. In some embodiments, each wing region independently comprises 17 consecutive natural phosphate linkages. In some embodiments, each wing region independently comprises 18 consecutive natural phosphate linkages. In some embodiments, each wing region independently comprises 19 consecutive natural phosphate linkages. In some embodiments, each wing region independently comprises 20 consecutive natural phosphate linkages.

In some embodiments, a wing is to the 5′-end of a core (5′-end wing). In some embodiments, a wing is to the 3′-end of a core (3′-end wing).

In some embodiments, a 5′-end wing comprises one or more modified internucleotidic linkages and one or more natural phosphate internucleotidic linkages. In some embodiments, a 3′-end wing comprises one or more modified internucleotidic linkages and one or more natural phosphate internucleotidic linkages. In some embodiments, each wing independently comprises one or more modified internucleotidic linkages and one or more natural phosphate internucleotidic linkages.

In some embodiments, a 5′-end wing comprises a modified internucleotidic linkage having one or more natural phosphate linkages connecting two or more nucleosides after (to the 3′-end) the modified internucleotidic linkage in the 5′-end wing. For example, a 5′-end wing mG*SmGmCmAmC comprises a modified internucleotidic linkage (mG*SmG) which has three natural phosphate linkages connecting four nucleosides (mGmCmAmC) after the modified internucleotidic linkage in the 5′-end wing. In some embodiments, a 5′-end wing comprises a modified internucleotidic linkages followed by one or more natural phosphate linkages and/or one or more modified internucleotidic linkages, which are followed by one or more natural phosphate linkages in the 5′-end wing (for example, mG*SmG and mG*SmC in mG*SmG*SmCmAmC). In some embodiments, a 5′-end wing comprises a modified internucleotidic linkages followed by one or more natural phosphate linkages in the 5′-end wing. In some embodiments, a 5′-end wing comprises a modified internucleotidic linkages followed by one or more consecutive natural phosphate linkages in the 5′-end wing. In some embodiments, a 5′-end wing comprises a natural phosphate linkage between the two nucleosides at its 3′-end. For example, a 5′-end wing mG*SmGmCmAmC has a natural phosphate linkage between the two nucleosides at its 3′-end (mG*SmGmCmAmC).

In some embodiments, a 3′-end wing comprises a modified internucleotidic linkage having one or more natural phosphate linkages connecting two or more nucleosides before (to the 5′-end) the modified internucleotidic linkage in the 3′-end wing. For example, a 3′-end wing mAmCmUmU*SmC comprises a modified internucleotidic linkage (mU*SmC) which has three natural phosphate linkages connecting four nucleosides (mAmCmUmU) before the modified internucleotidic linkage in the 3′-end wing. In some embodiments, a 3′-end wing comprises a modified internucleotidic linkages preceded by one or more natural phosphate linkages and/or one or more modified internucleotidic linkages, which are preceded by one or more natural phosphate linkages in the 3′-end wing (for example, mU*SmU and mU*SmC in mAmCmU*SmU*SmC). In some embodiments, a 3′-end wing comprises a modified internucleotidic linkages preceded by one or more natural phosphate linkages in the 3′-end wing. In some embodiments, a 3′-end wing comprises a modified internucleotidic linkages preceded by one or more consecutive natural phosphate linkages in the 3′-end wing. In some embodiments, a 3′-end wing comprises a natural phosphate linkage between the two nucleosides at its 5′-end. For example, a 3′-end wing having the structure of mAmCmUmU*SmC has a natural phosphate linkage between the two nucleosides at its 5′-end (mAmCmUmU*SmC).

In some embodiments, one or more is one. In some embodiments, one or more is two. In some embodiments, one or more is three. In some embodiments, one or more is four. In some embodiments, one or more is five. In some embodiments, one or more is six. In some embodiments, one or more is seven. In some embodiments, one or more is eight. In some embodiments, one or more is nine. In some embodiments, one or more is ten. In some embodiments, one or more is at least one. In some embodiments, one or more is at least two. In some embodiments, one or more is at least three. In some embodiments, one or more is at least four. In some embodiments, one or more is at least five. In some embodiments, one or more is at least six. In some embodiments, one or more is at least seven. In some embodiments, one or more is at least eight. In some embodiments, one or more is at least nine. In some embodiments, one or more is at least ten.

In some embodiments, a wing comprises only one chiral internucleotidic linkage. In some embodiments, a 5′-end wing comprises only one chiral internucleotidic linkage. In some embodiments, a 5′-end wing comprises only one chiral internucleotidic linkage at the 5′-end of the wing. In some embodiments, a 5′-end wing comprises only one chiral internucleotidic linkage at the 5′-end of the wing, and the chiral internucleotidic linkage is Rp. In some embodiments, a 5′-end wing comprises only one chiral internucleotidic linkage at the 5′-end of the wing, and the chiral internucleotidic linkage is Sp. In some embodiments, a 3′-end wing comprises only one chiral internucleotidic linkage at the 3′-end of the wing. In some embodiments, a 3′-end wing comprises only one chiral internucleotidic linkage at the 3′-end of the wing, and the chiral internucleotidic linkage is Rp. In some embodiments, a 3′-end wing comprises only one chiral internucleotidic linkage at the 3′-end of the wing, and the chiral internucleotidic linkage is Sp.

In some embodiments, a wing comprises two or more natural phosphate linkages. In some embodiments, all phosphate linkages within a wing are consecutive, and there are no non-phosphate linkages between any two phosphate linkages within a wing.

In some embodiments, a linkage connecting a wing and a core is considered part of the core when describing linkages, e.g., linkage chemistry, linkage stereochemistry, etc.

In some embodiments, a 5′-internucleotidic linkage connected to a sugar moiety without a 2′-modification is a modified linkage. In some embodiments, a 5′-internucleotidic linkage connected to a sugar moiety without a 2′-modification is a linkage having the structure of formula I. In some embodiments, a 5′-internucleotidic linkage connected to a sugar moiety without a 2′-modification is phosphorothioate linkage. In some embodiments, a 5′-internucleotidic linkage connected to a sugar moiety without a 2′-modification is a substituted phosphorothioate linkage. In some embodiments, a 5′-internucleotidic linkage connected to a sugar moiety without a 2′-modification is a phosphorothioate triester linkage. In some embodiments, each 5′-internucleotidic linkage connected to a sugar moiety without a 2′-modification is a modified linkage. In some embodiments, each 5′-internucleotidic linkage connected to a sugar moiety without a 2′-modification is a linkage having the structure of formula I. In some embodiments, each 5′-internucleotidic linkage connected to a sugar moiety without a 2′-modification is phosphorothioate linkage. In some embodiments, each 5′-internucleotidic linkage connected to a sugar moiety without a 2′-modification is a substituted phosphorothioate linkage. In some embodiments, each 5′-internucleotidic linkage connected to a sugar moiety without a 2′-modification is a phosphorothioate triester linkage.

In some embodiments, a 3′-internucleotidic linkage connected to a sugar moiety without a 2′-modification is a modified linkage. In some embodiments, a 3′-internucleotidic linkage connected to a sugar moiety without a 2′-modification is a linkage having the structure of formula I. In some embodiments, a 3′-internucleotidic linkage connected to a sugar moiety without a 2′-modification is phosphorothioate linkage. In some embodiments, a 3′-internucleotidic linkage connected to a sugar moiety without a 2′-modification is a substituted phosphorothioate linkage. In some embodiments, a 3′-internucleotidic linkage connected to a sugar moiety without a 2′-modification is a phosphorothioate triester linkage. In some embodiments, each 3′-internucleotidic linkage connected to a sugar moiety without a 2′-modification is a modified linkage. In some embodiments, each 3′-internucleotidic linkage connected to a sugar moiety without a 2′-modification is a linkage having the structure of formula I. In some embodiments, each 3′-internucleotidic linkage connected to a sugar moiety without a 2′-modification is phosphorothioate linkage. In some embodiments, each 3′-internucleotidic linkage connected to a sugar moiety without a 2′-modification is a substituted phosphorothioate linkage. In some embodiments, each 3′-internucleotidic linkage connected to a sugar moiety without a 2′-modification is a phosphorothioate triester linkage.

In some embodiments, both internucleotidic linkages connected to a sugar moiety without a 2′-modification are modified linkages. In some embodiments, both internucleotidic linkages connected to a sugar moiety without a 2′-modification are linkage having the structure of formula I. In some embodiments, both internucleotidic linkages connected to a sugar moiety without a 2′-modification are phosphorothioate linkages. In some embodiments, both internucleotidic linkages connected to a sugar moiety without a 2′-modification are substituted phosphorothioate linkages. In some embodiments, both internucleotidic linkages connected to a sugar moiety without a 2′-modification are phosphorothioate triester linkages. In some embodiments, each internucleotidic linkage connected to a sugar moiety without a 2′-modification is a modified linkage. In some embodiments, each internucleotidic linkage connected to a sugar moiety without a 2′-modification is a linkage having the structure of formula I. In some embodiments, each internucleotidic linkage connected to a sugar moiety without a 2′-modification is phosphorothioate linkage. In some embodiments, each internucleotidic linkage connected to a sugar moiety without a 2′-modification is a substituted phosphorothioate linkage. In some embodiments, each internucleotidic linkage connected to a sugar moiety without a 2′-modification is a phosphorothioate triester linkage.

In some embodiments, a sugar moiety without a 2′-modification is a sugar moiety found in a natural DNA nucleoside.

In some embodiments, for a wing-core-wing structure, the 5′-end wing comprises only one chiral internucleotidic linkage. In some embodiments, for a wing-core-wing structure, the 5′-end wing comprises only one chiral internucleotidic linkage at the 5′-end of the wing. In some embodiments, for a wing-core-wing structure, the 3′-end wing comprises only one chiral internucleotidic linkage. In some embodiments, for a wing-core-wing structure, the 3′-end wing comprises only one chiral internucleotidic linkage at the 3′-end of the wing. In some embodiments, for a wing-core-wing structure, each wing comprises only one chiral internucleotidic linkage. In some embodiments, for a wing-core-wing structure, each wing comprises only one chiral internucleotidic linkage, wherein the 5′-end wing comprises only one chiral internucleotidic linkage at its 5′-end; and the 3′-end wing comprises only one chiral internucleotidic linkage at its 3′-end. In some embodiments, the only chiral internucleotidic linkage in the 5′-wing is Rp. In some embodiments, the only chiral internucleotidic linkage in the 5′-wing is Sp. In some embodiments, the only chiral internucleotidic linkage in the 3′-wing is Rp. In some embodiments, the only chiral internucleotidic linkage in the 3′-wing is Sp. In some embodiments, the only chiral internucleotidic linkage in both the 5′- and the 3′-wings are Sp. In some embodiments, the only chiral internucleotidic linkage in both the 5′- and the 3′-wings are Rp. In some embodiments, the only chiral internucleotidic linkage in the 5′-wing is Sp, and the only chiral internucleotidic linkage in the 3′-wing is Rp. In some embodiments, the only chiral internucleotidic linkage in the 5′-wing is Rp, and the only chiral internucleotidic linkage in the 3′-wing is Sp.

In some embodiments, a wing comprises two chiral internucleotidic linkages. In some embodiments, a wing comprises only two chiral internucleotidic linkages, and one or more natural phosphate linkages. In some embodiments, a wing comprises only two chiral internucleotidic linkages, and two or more natural phosphate linkages. In some embodiments, a wing comprises only two chiral internucleotidic linkages, and two or more consecutive natural phosphate linkages. In some embodiments, a wing comprises only two chiral internucleotidic linkages, and two consecutive natural phosphate linkages. In some embodiments, a wing comprises only two chiral internucleotidic linkages, and three consecutive natural phosphate linkages. In some embodiments, a 5′-wing (to a core) comprises only two chiral internucleotidic linkages, one at its 5′-end and the other at its 3′-end, with one or more natural phosphate linkages in between. In some embodiments, a 5′-wing (to a core) comprises only two chiral internucleotidic linkages, one at its 5′-end and the other at its 3′-end, with two or more natural phosphate linkages in between. In some embodiments, a 3′-wing (to a core) comprises only two chiral internucleotidic linkages, one at its 3′-end and the other at its 3′-end, with one or more natural phosphate linkages in between. In some embodiments, a 3′-wing (to a core) comprises only two chiral internucleotidic linkages, one at its 3′-end and the other at its 3′-end, with two or more natural phosphate linkages in between.

In some embodiments, a 5′-wing comprises only two chiral internucleotidic linkages, one at its 5′-end and the other at its 3′-end, with one or more natural phosphate linkages in between, and the 3′-wing comprise only one internucleotidic linkage at its 3′-end. In some embodiments, a 5′-wing (to a core) comprises only two chiral internucleotidic linkages, one at its 5′-end and the other at its 3′-end, with two or more natural phosphate linkages in between, and the 3′-wing comprise only one internucleotidic linkage at its 3′-end. In some embodiments, each chiral internucleotidic linkage independently has its own stereochemistry. In some embodiments, both chiral internucleotidic linkages in the 5′-wing have the same stereochemistry. In some embodiments, both chiral internucleotidic linkages in the 5′-wing have different stereochemistry. In some embodiments, both chiral internucleotidic linkages in the 5′-wing are Rp. In some embodiments, both chiral internucleotidic linkages in the 5′-wing are Sp. In some embodiments, chiral internucleotidic linkages in the 5′- and 3′-wings have the same stereochemistry. In some embodiments, chiral internucleotidic linkages in the 5′- and 3′-wings are Rp. In some embodiments, chiral internucleotidic linkages in the 5′- and 3′-wings are Sp. In some embodiments, chiral internucleotidic linkages in the 5′- and 3′-wings have different stereochemistry.

In some embodiments, a chiral, modified phosphate linkage is a chiral phosphorothioate linkage, i.e., phosphorothioate internucleotidic linkage. In some embodiments, a wing region comprises at least 5%, 10%, 15%, 20%, 25%, 30%, 35%, 40%, 45%, 50%, 55%, 60%, 65%, 70%, 75%, 80%, 85%, 90%, 95%, or 100% chiral phosphorothioate internucleotidic linkages. In some embodiments, all chiral, modified phosphate linkages are chiral phosphorothioate internucleotidic linkages. In some embodiments, at least about 10%, 20%, 30%, 40%, 50%, 60%, 70%, 80%, or 90% chiral phosphorothioate internucleotidic linkages of a wing region are of the Sp conformation. In some embodiments, at least about 10% chiral phosphorothioate internucleotidic linkages of a wing region are of the Sp conformation. In some embodiments, at least about 20% chiral phosphorothioate internucleotidic linkages of a wing region are of the Sp conformation. In some embodiments, at least about 30% chiral phosphorothioate internucleotidic linkages of a wing region are of the Sp conformation. In some embodiments, at least about 40% chiral phosphorothioate internucleotidic linkages of a wing region are of the Sp conformation. In some embodiments, at least about 50% chiral phosphorothioate internucleotidic linkages of a wing region are of the Sp conformation. In some embodiments, at least about 60% chiral phosphorothioate internucleotidic linkages of a wing region are of the Sp conformation. In some embodiments, at least about 70% chiral phosphorothioate internucleotidic linkages of a wing region are of the Sp conformation. In some embodiments, at least about 80% chiral phosphorothioate internucleotidic linkages of a wing region are of the Sp conformation. In some embodiments, at least about 90% chiral phosphorothioate internucleotidic linkages of a wing region are of the Sp conformation. In some embodiments, at least about 95% chiral phosphorothioate internucleotidic linkages of a wing region are of the Sp conformation.

In some embodiments, at least about 1 chiral phosphorothioate internucleotidic linkage of a wing region is of the Sp conformation. In some embodiments, at least about 2 chiral phosphorothioate internucleotidic linkages of a wing region are of the Sp conformation. In some embodiments, at least about 3 chiral phosphorothioate internucleotidic linkages of a wing region are of the Sp conformation. In some embodiments, at least about 4 chiral phosphorothioate internucleotidic linkages of a wing region are of the Sp conformation. In some embodiments, at least about 5 chiral phosphorothioate internucleotidic linkages of a wing region are of the Sp conformation. In some embodiments, at least about 6 chiral phosphorothioate internucleotidic linkages of a wing region are of the Sp conformation. In some embodiments, at least about 7 chiral phosphorothioate internucleotidic linkages of a wing region are of the Sp conformation. In some embodiments, at least about 8 chiral phosphorothioate internucleotidic linkages of a wing region are of the Sp conformation. In some embodiments, at least about 9 chiral phosphorothioate internucleotidic linkages of a wing region are of the Sp conformation.

In some embodiments, at least about 2 consecutive chiral phosphorothioate internucleotidic linkages of a wing region are of the Sp conformation. In some embodiments, at least about 3 consecutive chiral phosphorothioate internucleotidic linkages of a wing region are of the Sp conformation. In some embodiments, at least about 4 consecutive chiral phosphorothioate internucleotidic linkages of a wing region are of the Sp conformation. In some embodiments, at least about 5 consecutive chiral phosphorothioate internucleotidic linkages of a wing region are of the Sp conformation. In some embodiments, at least about 6 consecutive chiral phosphorothioate internucleotidic linkages of a wing region are of the Sp conformation. In some embodiments, at least about 7 consecutive chiral phosphorothioate internucleotidic linkages of a wing region are of the Sp conformation. In some embodiments, at least about 8 consecutive chiral phosphorothioate internucleotidic linkages of a wing region are of the Sp conformation. In some embodiments, at least about 9 consecutive chiral phosphorothioate internucleotidic linkages of a wing region are of the Sp conformation.

In some embodiments, at least about 10%, 20%, 30%, 40%, 50%, 60%, 70%, 80%, or 90% chiral phosphorothioate internucleotidic linkages of a wing region are of the Rp conformation. In some embodiments, at least about 10% chiral phosphorothioate internucleotidic linkages of a wing region are of the Rp conformation. In some embodiments, at least about 20% chiral phosphorothioate internucleotidic linkages of a wing region are of the Rp conformation. In some embodiments, at least about 30% chiral phosphorothioate internucleotidic linkages of a wing region are of the Rp conformation. In some embodiments, at least about 40% chiral phosphorothioate internucleotidic linkages of a wing region are of the Rp conformation. In some embodiments, at least about 50% chiral phosphorothioate internucleotidic linkages of a wing region are of the Rp conformation. In some embodiments, at least about 60% chiral phosphorothioate internucleotidic linkages of a wing region are of the Rp conformation. In some embodiments, at least about 70% chiral phosphorothioate internucleotidic linkages of a wing region are of the Rp conformation. In some embodiments, at least about 80% chiral phosphorothioate internucleotidic linkages of a wing region are of the Rp conformation. In some embodiments, at least about 90% chiral phosphorothioate internucleotidic linkages of a wing region are of the Rp conformation. In some embodiments, at least about 95% chiral phosphorothioate internucleotidic linkages of a wing region are of the Rp conformation.

In some embodiments, less than about 10%, 20%, 30%, 40%, 50%, 60%, 70%, 80%, or 90% chiral phosphorothioate internucleotidic linkages of a wing region are of the Rp conformation. In some embodiments, less than about 10% chiral phosphorothioate internucleotidic linkages of a wing region are of the Rp conformation. In some embodiments, less than about 20% chiral phosphorothioate internucleotidic linkages of a wing region are of the Rp conformation. In some embodiments, less than about 30% chiral phosphorothioate internucleotidic linkages of a wing region are of the Rp conformation. In some embodiments, less than about 40% chiral phosphorothioate internucleotidic linkages of a wing region are of the Rp conformation. In some embodiments, less than about 50% chiral phosphorothioate internucleotidic linkages of a wing region are of the Rp conformation. In some embodiments, less than about 60% chiral phosphorothioate internucleotidic linkages of a wing region are of the Rp conformation. In some embodiments, less than about 70% chiral phosphorothioate internucleotidic linkages of a wing region are of the Rp conformation. In some embodiments, less than about 80% chiral phosphorothioate internucleotidic linkages of a wing region are of the Rp conformation. In some embodiments, less than about 90% chiral phosphorothioate internucleotidic linkages of a wing region are of the Rp conformation. In some embodiments, less than about 95% chiral phosphorothioate internucleotidic linkages of a wing region are of the Rp conformation. In some embodiments, a wing region has only one Rp chiral phosphorothioate internucleotidic linkages. In some embodiments, a wing region has only one Rp chiral phosphorothioate internucleotidic linkages, wherein all internucleotide linkages are chiral phosphorothioate internucleotidic linkages.

In some embodiments, at least about 1 chiral phosphorothioate internucleotidic linkage of a wing region is of the Rp conformation. In some embodiments, at least about 2 chiral phosphorothioate internucleotidic linkages of a wing region are of the Rp conformation. In some embodiments, at least about 3 chiral phosphorothioate internucleotidic linkages of a wing region are of the Rp conformation. In some embodiments, at least about 4 chiral phosphorothioate internucleotidic linkages of a wing region are of the Rp conformation. In some embodiments, at least about 5 chiral phosphorothioate internucleotidic linkages of a wing region are of the Rp conformation. In some embodiments, at least about 6 chiral phosphorothioate internucleotidic linkages of a wing region are of the Rp conformation. In some embodiments, at least about 7 chiral phosphorothioate internucleotidic linkages of a wing region are of the Rp conformation. In some embodiments, at least about 8 chiral phosphorothioate internucleotidic linkages of a wing region are of the Rp conformation. In some embodiments, at least about 9 chiral phosphorothioate internucleotidic linkages of a wing region are of the Rp conformation.

In some embodiments, at least about 2 consecutive chiral phosphorothioate internucleotidic linkages of a wing region are of the Rp conformation. In some embodiments, at least about 3 consecutive chiral phosphorothioate internucleotidic linkages of a wing region are of the Rp conformation. In some embodiments, at least about 4 consecutive chiral phosphorothioate internucleotidic linkages of a wing region are of the Rp conformation. In some embodiments, at least about 5 consecutive chiral phosphorothioate internucleotidic linkages of a wing region are of the Rp conformation. In some embodiments, at least about 6 consecutive chiral phosphorothioate internucleotidic linkages of a wing region are of the Rp conformation. In some embodiments, at least about 7 consecutive chiral phosphorothioate internucleotidic linkages of a wing region are of the Rp conformation. In some embodiments, at least about 8 consecutive chiral phosphorothioate internucleotidic linkages of a wing region are of the Rp conformation. In some embodiments, at least about 9 consecutive chiral phosphorothioate internucleotidic linkages of a wing region are of the Rp conformation.

In some embodiments, a wing comprises one or more modified sugar moieties. In some embodiments, a wing comprises two or more modified sugar moieties. In some embodiments, a wing comprises three or more modified sugar moieties. In some embodiments, a wing comprises four or more modified sugar moieties. In some embodiments, a wing comprises five or more modified sugar moieties. In some embodiments, a wing comprises six or more modified sugar moieties. In some embodiments, a wing comprises seven or more modified sugar moieties. In some embodiments, a wing comprises eight or more modified sugar moieties. In some embodiments, a wing comprises nine or more modified sugar moieties. In some embodiments, a wing comprises ten or more modified sugar moieties. In some embodiments, a wing comprises 11 or more modified sugar moieties. In some embodiments, a wing comprises 12 or more modified sugar moieties. In some embodiments, a wing comprises 13 or more modified sugar moieties. In some embodiments, a wing comprises 14 or more modified sugar moieties. In some embodiments, a wing comprises 15 or more modified sugar moieties. In some embodiments, a wing comprises 16 or more modified sugar moieties. In some embodiments, a wing comprises 17 or more modified sugar moieties. In some embodiments, a wing comprises 18 or more modified sugar moieties. In some embodiments, a wing comprises 19 or more modified sugar moieties. In some embodiments, a wing comprises 20 or more modified sugar moieties. In some embodiments, a wing comprises 21 or more modified sugar moieties. In some embodiments, a wing comprises 22 or more modified sugar moieties. In some embodiments, a wing comprises 23 or more modified sugar moieties. In some embodiments, a wing comprises 24 or more modified sugar moieties. In some embodiments, a wing comprises 25 or more modified sugar moieties. In some embodiments, a wing comprises 30 or more modified sugar moieties. In some embodiments, a wing comprises 35 or more modified sugar moieties.

In some embodiments, a wing comprises one or more 2′-modified sugar moieties. In some embodiments, a wing comprises two or more 2′-modified sugar moieties. In some embodiments, a wing comprises three or more 2′-modified sugar moieties. In some embodiments, a wing comprises four or more 2′-modified sugar moieties. In some embodiments, a wing comprises five or more 2′-modified sugar moieties. In some embodiments, a wing comprises six or more 2′-modified sugar moieties. In some embodiments, a wing comprises seven or more 2′-modified sugar moieties. In some embodiments, a wing comprises eight or more 2′-modified sugar moieties. In some embodiments, a wing comprises nine or more 2′-modified sugar moieties. In some embodiments, a wing comprises ten or more 2′-modified sugar moieties. In some embodiments, a wing comprises 11 or more 2′-modified sugar moieties. In some embodiments, a wing comprises 12 or more 2′-modified sugar moieties. In some embodiments, a wing comprises 13 or more 2′-modified sugar moieties. In some embodiments, a wing comprises 14 or more 2′-modified sugar moieties. In some embodiments, a wing comprises 15 or more 2′-modified sugar moieties. In some embodiments, a wing comprises 16 or more 2′-modified sugar moieties. In some embodiments, a wing comprises 17 or more 2′-modified sugar moieties. In some embodiments, a wing comprises 18 or more 2′-modified sugar moieties. In some embodiments, a wing comprises 19 or more 2′-modified sugar moieties. In some embodiments, a wing comprises 20 or more 2′-modified sugar moieties. In some embodiments, a wing comprises 21 or more 2′-modified sugar moieties. In some embodiments, a wing comprises 22 or more 2′-modified sugar moieties. In some embodiments, a wing comprises 23 or more 2′-modified sugar moieties. In some embodiments, a wing comprises 24 or more 2′-modified sugar moieties. In some embodiments, a wing comprises 25 or more 2′-modified sugar moieties. In some embodiments, a wing comprises 30 or more 2′-modified sugar moieties. In some embodiments, a wing comprises 35 or more 2′-modified sugar moieties.

In some embodiments, a wing comprises one or more 2′-F. In some embodiments, a wing comprises two or more 2′-F. In some embodiments, a wing comprises three or more 2′-F. In some embodiments, a wing comprises four or more 2′-F. In some embodiments, a wing comprises five or more 2′-F. In some embodiments, a wing comprises six or more 2′-F. In some embodiments, a wing comprises seven or more 2′-F. In some embodiments, a wing comprises eight or more 2′-F. In some embodiments, a wing comprises nine or more 2′-F. In some embodiments, a wing comprises ten or more 2′-F. In some embodiments, a wing comprises 11 or more 2′-F. In some embodiments, a wing comprises 12 or more 2′-F. In some embodiments, a wing comprises 13 or more 2′-F. In some embodiments, a wing comprises 14 or more 2′-F. In some embodiments, a wing comprises 15 or more 2′-F. In some embodiments, a wing comprises 16 or more 2′-F. In some embodiments, a wing comprises 17 or more 2′-F. In some embodiments, a wing comprises 18 or more 2′-F. In some embodiments, a wing comprises 19 or more 2′-F. In some embodiments, a wing comprises 20 or more 2′-F. In some embodiments, a wing comprises 21 or more 2′-F. In some embodiments, a wing comprises 22 or more 2′-F. In some embodiments, a wing comprises 23 or more 2′-F. In some embodiments, a wing comprises 24 or more 2′-F. In some embodiments, a wing comprises 25 or more 2′-F. In some embodiments, a wing comprises 30 or more 2′-F. In some embodiments, a wing comprises 35 or more 2′-F.

In some embodiments, a wing comprises one 2′-F. In some embodiments, a wing comprises two 2′-F. In some embodiments, a wing comprises three 2′-F. In some embodiments, a wing comprises four 2′-F. In some embodiments, a wing comprises five 2′-F. In some embodiments, a wing comprises six 2′-F. In some embodiments, a wing comprises seven 2′-F. In some embodiments, a wing comprises eight 2′-F. In some embodiments, a wing comprises nine 2′-F. In some embodiments, a wing comprises ten 2′-F. In some embodiments, a wing comprises 11 2′-F. In some embodiments, a wing comprises 12 2′-F. In some embodiments, a wing comprises 13 2′-F. In some embodiments, a wing comprises 14 2′-F. In some embodiments, a wing comprises 15 2′-F. In some embodiments, a wing comprises 16 2′-F. In some embodiments, a wing comprises 17 2′-F. In some embodiments, a wing comprises 18 2′-F. In some embodiments, a wing comprises 19 2′-F. In some embodiments, a wing comprises 20 2′-F. In some embodiments, a wing comprises 21 2′-F. In some embodiments, a wing comprises 22 2′-F. In some embodiments, a wing comprises 23 2′-F. In some embodiments, a wing comprises 24 2′-F. In some embodiments, a wing comprises 25 2′-F. In some embodiments, a wing comprises 30 2′-F. In some embodiments, a wing comprises 35 2′-F.

In some embodiments, a wing comprises one or more consecutive 2′-F. In some embodiments, a wing comprises two or more consecutive 2′-F. In some embodiments, a wing comprises three or more consecutive 2′-F. In some embodiments, a wing comprises four or more consecutive 2′-F. In some embodiments, a wing comprises five or more consecutive 2′-F. In some embodiments, a wing comprises six or more consecutive 2′-F. In some embodiments, a wing comprises seven or more consecutive 2′-F. In some embodiments, a wing comprises eight or more consecutive 2′-F. In some embodiments, a wing comprises nine or more consecutive 2′-F. In some embodiments, a wing comprises ten or more consecutive 2′-F. In some embodiments, a wing comprises 11 or more consecutive 2′-F. In some embodiments, a wing comprises 12 or more consecutive 2′-F. In some embodiments, a wing comprises 13 or more consecutive 2′-F. In some embodiments, a wing comprises 14 or more consecutive 2′-F. In some embodiments, a wing comprises 15 or more consecutive 2′-F. In some embodiments, a wing comprises 16 or more consecutive 2′-F. In some embodiments, a wing comprises 17 or more consecutive 2′-F. In some embodiments, a wing comprises 18 or more consecutive 2′-F. In some embodiments, a wing comprises 19 or more consecutive 2′-F. In some embodiments, a wing comprises 20 or more consecutive 2′-F. In some embodiments, a wing comprises 21 or more consecutive 2′-F. In some embodiments, a wing comprises 22 or more consecutive 2′-F. In some embodiments, a wing comprises 23 or more consecutive 2′-F. In some embodiments, a wing comprises 24 or more consecutive 2′-F. In some embodiments, a wing comprises 25 or more consecutive 2′-F. In some embodiments, a wing comprises 30 or more consecutive 2′-F. In some embodiments, a wing comprises 35 or more consecutive 2′-F.

In some embodiments, a wing comprises one consecutive 2′-F. In some embodiments, a wing comprises two consecutive 2′-F. In some embodiments, a wing comprises three consecutive 2′-F. In some embodiments, a wing comprises four consecutive 2′-F. In some embodiments, a wing comprises five consecutive 2′-F. In some embodiments, a wing comprises six consecutive 2′-F. In some embodiments, a wing comprises seven consecutive 2′-F. In some embodiments, a wing comprises eight consecutive 2′-F. In some embodiments, a wing comprises nine consecutive 2′-F. In some embodiments, a wing comprises ten consecutive 2′-F. In some embodiments, a wing comprises 11 consecutive 2′-F. In some embodiments, a wing comprises 12 consecutive 2′-F. In some embodiments, a wing comprises 13 consecutive 2′-F. In some embodiments, a wing comprises 14 consecutive 2′-F. In some embodiments, a wing comprises 15 consecutive 2′-F. In some embodiments, a wing comprises 16 consecutive 2′-F. In some embodiments, a wing comprises 17 consecutive 2′-F. In some embodiments, a wing comprises 18 consecutive 2′-F. In some embodiments, a wing comprises 19 consecutive 2′-F. In some embodiments, a wing comprises 20 consecutive 2′-F. In some embodiments, a wing comprises 21 consecutive 2′-F. In some embodiments, a wing comprises 22 consecutive 2′-F. In some embodiments, a wing comprises 23 consecutive 2′-F. In some embodiments, a wing comprises 24 consecutive 2′-F. In some embodiments, a wing comprises 25 consecutive 2′-F. In some embodiments, a wing comprises 30 consecutive 2′-F. In some embodiments, a wing comprises 35 consecutive 2′-F.

In some embodiments, a core region has a length of one or more bases. In some embodiments, a core region has a length of two or more bases. In some embodiments, a core region has a length of three or more bases. In some embodiments, a core region has a length of four or more bases. In some embodiments, a core region has a length of five or more bases. In some embodiments, a core region has a length of six or more bases. In some embodiments, a core region has a length of seven or more bases. In some embodiments, a core region has a length of eight or more bases. In some embodiments, a core region has a length of nine or more bases. In some embodiments, a core region has a length of ten or more bases. In some embodiments, a core region has a length of 11, 12, 13, 14, 15, 16, 17, 18, 19, 20, 25, or more bases. In certain embodiments, a core region has a length of 11 or more bases. In certain embodiments, a core region has a length of 12 or more bases. In certain embodiments, a core region has a length of 13 or more bases. In certain embodiments, a core region has a length of 14 or more bases. In certain embodiments, a core region has a length of 15 or more bases. In certain embodiments, a core region has a length of 16 or more bases. In certain embodiments, a core region has a length of 17 or more bases. In certain embodiments, a core region has a length of 18 or more bases. In certain embodiments, a core region has a length of 19 or more bases. In certain embodiments, a core region has a length of 20 or more bases. In certain embodiments, a core region has a length of more than 20 bases. In certain embodiments, a core region has a length of 2 bases. In certain embodiments, a core region has a length of 3 bases. In certain embodiments, a core region has a length of 4 bases. In certain embodiments, a core region has a length of 5 bases. In certain embodiments, a core region has a length of 6 bases. In certain embodiments, a core region has a length of 7 bases. In certain embodiments, a core region has a length of 8 bases. In certain embodiments, a core region has a length of 9 bases. In certain embodiments, a core region has a length of 10 bases. In certain embodiments, a core region has a length of 11 bases. In certain embodiments, a core region has a length of 12 bases. In certain embodiments, a core region has a length of 13 bases. In certain embodiments, a core region has a length of 14 bases. In certain embodiments, a core region has a length of 15 bases. In certain embodiments, a core region has a length of 16 bases. In certain embodiments, a core region has a length of 17 bases. In certain embodiments, a core region has a length of 18 bases. In certain embodiments, a core region has a length of 19 bases. In certain embodiments, a core region has a length of 20 bases.

In some embodiments, a core comprises one or more modified internucleotidic linkages. In some embodiments, a core comprises one or more natural phosphate linkages. In some embodiments, a core independently comprises one or more modified internucleotidic linkages and one or more natural phosphate linkages. In some embodiments, a core comprises no natural phosphate linkages. In some embodiments, each core linkage is a modified internucleotidic linkage.

In some embodiments, a core comprises at least one natural phosphate linkage. In some embodiments, a core comprises at least two modified internucleotidic linkages. In some embodiments, a core comprises at least three modified internucleotidic linkages. In some embodiments, a core comprises at least four modified internucleotidic linkages. In some embodiments, a core comprises at least five modified internucleotidic linkages. In some embodiments, a core comprises at least six modified internucleotidic linkages. In some embodiments, a core comprises at least seven modified internucleotidic linkages. In some embodiments, a core comprises at least eight modified internucleotidic linkages. In some embodiments, a core comprises at least nine modified internucleotidic linkages. In some embodiments, a core comprises at least ten modified internucleotidic linkages. In some embodiments, a core comprises at least 11 modified internucleotidic linkages. In some embodiments, a core comprises at least 12 modified internucleotidic linkages. In some embodiments, a core comprises at least 13 modified internucleotidic linkages. In some embodiments, a core comprises at least 14 modified internucleotidic linkages. In some embodiments, a core comprises at least 15 modified internucleotidic linkages. In some embodiments, a core comprises at least 16 modified internucleotidic linkages. In some embodiments, a core comprises at least 17 modified internucleotidic linkages. In some embodiments, a core comprises at least 18 modified internucleotidic linkages. In some embodiments, a core comprises at least 19 modified internucleotidic linkages. In some embodiments, a core comprises at least 20 modified internucleotidic linkages.

In some embodiments, a core comprises one or more chiral internucleotidic linkages. In some embodiments, a core comprises one or more natural phosphate linkages. In some embodiments, a core independently comprises one or more chiral internucleotidic linkages and one or more natural phosphate linkages. In some embodiments, a core comprises no natural phosphate linkages. In some embodiments, each core linkage is a chiral internucleotidic linkage.

In some embodiments, a core comprises at least one natural phosphate linkage. In some embodiments, a core comprises at least two chiral internucleotidic linkages. In some embodiments, a core comprises at least three chiral internucleotidic linkages. In some embodiments, a core comprises at least four chiral internucleotidic linkages. In some embodiments, a core comprises at least five chiral internucleotidic linkages. In some embodiments, a core comprises at least six chiral internucleotidic linkages. In some embodiments, a core comprises at least seven chiral internucleotidic linkages. In some embodiments, a core comprises at least eight chiral internucleotidic linkages. In some embodiments, a core comprises at least nine chiral internucleotidic linkages. In some embodiments, a core comprises at least ten chiral internucleotidic linkages. In some embodiments, a core comprises at least 11 chiral internucleotidic linkages. In some embodiments, a core comprises at least 12 chiral internucleotidic linkages. In some embodiments, a core comprises at least 13 chiral internucleotidic linkages. In some embodiments, a core comprises at least 14 chiral internucleotidic linkages. In some embodiments, a core comprises at least 15 chiral internucleotidic linkages. In some embodiments, a core comprises at least 16 chiral internucleotidic linkages. In some embodiments, a core comprises at least 17 chiral internucleotidic linkages. In some embodiments, a core comprises at least 18 chiral internucleotidic linkages. In some embodiments, a core comprises at least 19 chiral internucleotidic linkages. In some embodiments, a core comprises at least 20 chiral internucleotidic linkages.

In some embodiments, a core comprises one natural phosphate linkage. In some embodiments, a core comprises two chiral internucleotidic linkages. In some embodiments, a core comprises three chiral internucleotidic linkages. In some embodiments, a core comprises four chiral internucleotidic linkages. In some embodiments, a core comprises five chiral internucleotidic linkages. In some embodiments, a core comprises six chiral internucleotidic linkages. In some embodiments, a core comprises seven chiral internucleotidic linkages. In some embodiments, a core comprises eight chiral internucleotidic linkages. In some embodiments, a core comprises nine chiral internucleotidic linkages. In some embodiments, a core comprises ten chiral internucleotidic linkages. In some embodiments, a core comprises 11 chiral internucleotidic linkages. In some embodiments, a core comprises 12 chiral internucleotidic linkages. In some embodiments, a core comprises 13 chiral internucleotidic linkages. In some embodiments, a core comprises 14 chiral internucleotidic linkages. In some embodiments, a core comprises 15 chiral internucleotidic linkages. In some embodiments, a core comprises 16 chiral internucleotidic linkages. In some embodiments, a core comprises 17 chiral internucleotidic linkages. In some embodiments, a core comprises 18 chiral internucleotidic linkages. In some embodiments, a core comprises 19 chiral internucleotidic linkages. In some embodiments, a core comprises 20 chiral internucleotidic linkages.

In some embodiments, a core region has a pattern of backbone chiral centers comprising (Sp)m(Rp)n, (Rp)n(Sp)m, (Np)t(Rp)n(Sp)m, or (Sp)t(Rp)n(Sp)m, wherein each of m, n, t and Np is independently as defined and described in the present disclosure. In some embodiments, a core region has a pattern of backbone chiral centers comprising (Sp)m(Rp)n, (Rp)n(Sp)m, (Np)t(Rp)n(Sp)m, or (Sp)t(Rp)n(Sp)m. In some embodiments, a core region has a pattern of backbone chiral centers comprising (Sp)m(Rp)n. In some embodiments, a core region has a pattern of backbone chiral centers comprising (Sp)m(Rp)n, wherein m>2 and n is 1. In some embodiments, a core region has a pattern of backbone chiral centers comprising (Rp)n(Sp)m. In some embodiments, a core region has a pattern of backbone chiral centers comprising (Rp)n(Sp)m, wherein m>2 and n is 1. In some embodiments, a core region has a pattern of backbone chiral centers comprising (Np)t(Rp)n(Sp)m. In some embodiments, a core region has a pattern of backbone chiral centers comprising (Np)t(Rp)n(Sp)m, wherein m>2 and n is 1. In some embodiments, a core region has a pattern of backbone chiral centers comprising (Np)t(Rp)n(Sp)m, wherein t>2, m>2 and n is 1. In some embodiments, a core region has a pattern of backbone chiral centers comprising (Sp)t(Rp)n(Sp)m. In some embodiments, a core region has a pattern of backbone chiral centers comprising (Sp)t(Rp)n(Sp)m, wherein m>2 and n is 1. In some embodiments, a core region has a pattern of backbone chiral centers comprising (Sp)t(Rp)n(Sp)m, wherein t>2, m>2 and n is 1. Among other things, the present disclosure demonstrates that, in some embodiments, such patterns can provide and/or enhance controlled cleavage, improved cleavage rate, selectivity, etc., of a target sequence, e.g., an RNA sequence. Example patterns of backbone chiral centers are described in the present disclosure.

In some embodiments, at least 60% of the chiral internucleotidic linkages in the core region are Sp. In some embodiments, at least 65% of the chiral internucleotidic linkages in the core region are Sp. In some embodiments, at least 66% of the chiral internucleotidic linkages in the core region are Sp. In some embodiments, at least 67% of the chiral internucleotidic linkages in the core region are Sp. In some embodiments, at least 70% of the chiral internucleotidic linkages in the core region are Sp. In some embodiments, at least 75% of the chiral internucleotidic linkages in the core region are Sp. In some embodiments, at least 80% of the chiral internucleotidic linkages in the core region are Sp. In some embodiments, at least 85% of the chiral internucleotidic linkages in the core region are Sp. In some embodiments, at least 90% of the chiral internucleotidic linkages in the core region are Sp. In some embodiments, at least 95% of the chiral internucleotidic linkages in the core region are Sp. In some embodiments, each chiral internucleotidic linkages in the core region is Sp.

In some embodiments, at least 1 core region internucleotidic linkage is Sp. In some embodiments, at least 2 core region internucleotidic linkages are Sp. In some embodiments, at least 3 core region internucleotidic linkages are Sp. In some embodiments, at least 4 core region internucleotidic linkages are Sp. In some embodiments, at least 5 core region internucleotidic linkages are Sp. In some embodiments, at least 6 core region internucleotidic linkages are Sp. In some embodiments, at least 7 core region internucleotidic linkages are Sp. In some embodiments, at least 8 core region internucleotidic linkages are Sp. In some embodiments, at least 9 core region internucleotidic linkages are Sp. In some embodiments, at least 10 core region internucleotidic linkages are Sp. In some embodiments, at least 11 core region internucleotidic linkages are Sp. In some embodiments, at least 12 core region internucleotidic linkages are Sp. In some embodiments, at least 13 core region internucleotidic linkages are Sp. In some embodiments, at least 14 core region internucleotidic linkages are Sp. In some embodiments, at least 15 core region internucleotidic linkages are Sp. In some embodiments, at least 16 core region internucleotidic linkages are Sp. In some embodiments, at least 17 core region internucleotidic linkages are Sp. In some embodiments, at least 18 core region internucleotidic linkages are Sp. In some embodiments, at least 19 core region internucleotidic linkages are Sp. In some embodiments, at least 20 core region internucleotidic linkages are Sp. In some embodiments, at least 21 core region internucleotidic linkages are Sp. In some embodiments, at least two core region internucleotidic linkages are Sp. In some embodiments, the Sp internucleotidic linkages are consecutive.

In some embodiments, at least 60% of the chiral internucleotidic linkages in the core region are Rp. In some embodiments, at least 65% of the chiral internucleotidic linkages in the core region are Rp. In some embodiments, at least 66% of the chiral internucleotidic linkages in the core region are Rp. In some embodiments, at least 67% of the chiral internucleotidic linkages in the core region are Rp. In some embodiments, at least 70% of the chiral internucleotidic linkages in the core region are Rp. In some embodiments, at least 75% of the chiral internucleotidic linkages in the core region are Rp. In some embodiments, at least 80% of the chiral internucleotidic linkages in the core region are Rp. In some embodiments, at least 85% of the chiral internucleotidic linkages in the core region are Rp. In some emIn some embodiments, each chiral internucleotidic linkages in the core region is Rp.

In some embodiments, at least 1 core region internucleotidic linkage is Rp. In some embodiments, at least 2 core region internucleotidic linkages are Rp. In some embodiments, at least 3 core region internucleotidic linkages are Rp. In some embodiments, at least 4 core region internucleotidic linkages are Rp. In some embodiments, at least 5 core region internucleotidic linkages are Rp. In some embodiments, at least 6 core region internucleotidic linkages are Rp. In some embodiments, at least 7 core region internucleotidic linkages are Rp. In some embodiments, at least 8 core region internucleotidic linkages are Rp. In some embodiments, at least 9 core region internucleotidic linkages are Rp. In some embodiments, at least 10 core region internucleotidic linkages are Rp. In some embodiments, at least 11 core region internucleotidic linkages are Rp. In some embodiments, at least 12 core region internucleotidic linkages are Rp. In some embodiments, at least 13 core region internucleotidic linkages are Rp. In some embodiments, at least 14 core region internucleotidic linkages are Rp. In some embodiments, at least 15 core region internucleotidic linkages are Rp. In some embodiments, at least 16 core region internucleotidic linkages are Rp. In some embodiments, at least 17 core region internucleotidic linkages are Rp. In some embodiments, at least 18 core region internucleotidic linkages are Rp. In some embodiments, at least 19 core region internucleotidic linkages are Rp. In some embodiments, at least 20 core region internucleotidic linkages are Rp. In some embodiments, at least 21 core region internucleotidic linkages are Rp. In some embodiments, at least two core region internucleotidic linkages are Rp. In some embodiments, the Rp internucleotidic linkages are consecutive.

In some embodiments, a core comprises one or more modified sugar moieties. In some embodiments, a core comprises two or more modified sugar moieties. In some embodiments, a core comprises three or more modified sugar moieties. In some embodiments, a core comprises four or more modified sugar moieties. In some embodiments, a core comprises five or more modified sugar moieties. In some embodiments, a core comprises six or more modified sugar moieties. In some embodiments, a core comprises seven or more modified sugar moieties. In some embodiments, a core comprises eight or more modified sugar moieties. In some embodiments, a core comprises nine or more modified sugar moieties. In some embodiments, a core comprises ten or more modified sugar moieties. In some embodiments, a core comprises 11 or more modified sugar moieties. In some embodiments, a core comprises 12 or more modified sugar moieties. In some embodiments, a core comprises 13 or more modified sugar moieties. In some embodiments, a core comprises 14 or more modified sugar moieties. In some embodiments, a core comprises 15 or more modified sugar moieties. In some embodiments, a core comprises 16 or more modified sugar moieties. In some embodiments, a core comprises 17 or more modified sugar moieties. In some embodiments, a core comprises 18 or more modified sugar moieties. In some embodiments, a core comprises 19 or more modified sugar moieties. In some embodiments, a core comprises 20 or more modified sugar moieties. In some embodiments, a core comprises 21 or more modified sugar moieties. In some embodiments, a core comprises 22 or more modified sugar moieties. In some embodiments, a core comprises 23 or more modified sugar moieties. In some embodiments, a core comprises 24 or more modified sugar moieties. In some embodiments, a core comprises 25 or more modified sugar moieties. In some embodiments, a core comprises 30 or more modified sugar moieties. In some embodiments, a core comprises 35 or more modified sugar moieties. In some embodiments, a 2′-modification is 2′-OR¹. In some embodiments, a 2′-modification is 2′-OMe.

In some embodiments, a core comprises one or more 2′-modified sugar moieties. In some embodiments, a core comprises two or more 2′-modified sugar moieties. In some embodiments, a core comprises three or more 2′-modified sugar moieties. In some embodiments, a core comprises four or more 2′-modified sugar moieties. In some embodiments, a core comprises five or more 2′-modified sugar moieties. In some embodiments, a core comprises six or more 2′-modified sugar moieties. In some embodiments, a core comprises seven or more 2′-modified sugar moieties. In some embodiments, a core comprises eight or more 2′-modified sugar moieties. In some embodiments, a core comprises nine or more 2′-modified sugar moieties. In some embodiments, a core comprises ten or more 2′-modified sugar moieties. In some embodiments, a core comprises 11 or more 2′-modified sugar moieties. In some embodiments, a core comprises 12 or more 2′-modified sugar moieties. In some embodiments, a core comprises 13 or more 2′-modified sugar moieties. In some embodiments, a core comprises 14 or more 2′-modified sugar moieties. In some embodiments, a core comprises 15 or more 2′-modified sugar moieties. In some embodiments, a core comprises 16 or more 2′-modified sugar moieties. In some embodiments, a core comprises 17 or more 2′-modified sugar moieties. In some embodiments, a core comprises 18 or more 2′-modified sugar moieties. In some embodiments, a core comprises 19 or more 2′-modified sugar moieties. In some embodiments, a core comprises 20 or more 2′-modified sugar moieties. In some embodiments, a core comprises 21 or more 2′-modified sugar moieties. In some embodiments, a core comprises 22 or more 2′-modified sugar moieties. In some embodiments, a core comprises 23 or more 2′-modified sugar moieties. In some embodiments, a core comprises 24 or more 2′-modified sugar moieties. In some embodiments, a core comprises 25 or more 2′-modified sugar moieties. In some embodiments, a core comprises 30 or more 2′-modified sugar moieties. In some embodiments, a core comprises 35 or more 2′-modified sugar moieties. In some embodiments, a 2′-modification is 2′-OR¹. In some embodiments, a 2′-modification is 2′-OMe.

In some embodiments, a wing-core-wing (i.e., X—Y—X) motif is represented numerically as, e.g., 5-10-4, meaning the wing to the 5′-end of the core is 5 bases in length, the core region is 10 bases in length, and the wing region to the 3′-end of the core is 4-bases in length. In some embodiments, a wing-core-wing motif is any of, e.g. 2-16-2, 3-14-3, 4-12-4, 5-10-5, 2-9-6, 3-9-3, 3-9-4, 3-9-5, 4-7-4, 4-9-3, 4-9-4, 4-9-5, 4-10-5, 4-11-4, 4-11-5, 5- 7-5, 5-8-6, 8-7-5, 7-7-6, 5-9-3, 5-9-5, 5-10-4, 5-10-5, 6-7-6, 6-8-5, and 6-9-2, etc. In certain embodiments, a wing-core-wing motif is 5-10-5. In certain embodiments, a wing-core-wing motif is 7-7-6. In certain embodiments, a wing-core-wing motif is 8-7-5.

In some embodiments, a wing-core motif is 5-15, 6-14, 7-13, 8-12, 9-12, etc. In some embodiments, a core-wing motif is 5-15, 6-14, 7-13, 8-12, 9-12, etc.

In some embodiments, the internucleosidic linkages of provided oligonucleotides of such wing-core-wing (i.e., X—Y—X) motifs are all chiral, modified phosphate linkages. In some embodiments, the internucleosidic linkages of provided oligonucleotides of such wing-core-wing (i.e., X—Y—X) motifs are all chiral phosphorothioate internucleotidic linkages. In some embodiments, chiral internucleotidic linkages of provided oligonucleotides of such wing-core-wing motifs are at least about 10, 20, 30, 40, 50, 50, 70, 80, or 90% chiral, modified phosphate internucleotidic linkages. In some embodiments, chiral internucleotidic linkages of provided oligonucleotides of such wing-core-wing motifs are at least about 10, 20, 30, 40, 50, 60, 70, 80, or 90% chiral phosphorothioate internucleotidic linkages. In some embodiments, chiral internucleotidic linkages of provided oligonucleotides of such wing-core-wing motifs are at least about 10, 20, 30, 40, 50, 50, 70, 80, or 90% chiral phosphorothioate internucleotidic linkages of the Sp conformation.

In some embodiments, each wing region of a wing-core-wing motif optionally contains chiral, modified phosphate internucleotidic linkages. In some embodiments, each wing region of a wing-core-wing motif optionally contains chiral phosphorothioate internucleotidic linkages. In some embodiments, each wing region of a wing-core-wing motif contains chiral phosphorothioate internucleotidic linkages. In some embodiments, the two wing regions of a wing-core-wing motif have the same internucleotidic linkage stereochemistry. In some embodiments, the two wing regions have different internucleotidic linkage stereochemistry. In some embodiments, each internucleotidic linkage in the wings is independently a chiral internucleotidic linkage.

In some embodiments, the core region of a wing-core-wing motif optionally contains chiral, modified phosphate internucleotidic linkages. In some embodiments, the core region of a wing-core-wing motif optionally contains chiral phosphorothioate internucleotidic linkages. In some embodiments, the core region of a wing-core-wing motif comprises a repeating pattern of internucleotidic linkage stereochemistry. In some embodiments, the core region of a wing-core-wing motif has a repeating pattern of internucleotidic linkage stereochemistry. In some embodiments, the core region of a wing-core-wing motif comprises repeating pattern of internucleotidic linkage stereochemistry, wherein the repeating pattern is (Sp)mRp or Rp(Sp)m, wherein in is 1-50. In some embodiments, the core region of a wing-core-wing motif comprises repeating pattern of internucleotidic linkage stereochemistry, wherein the repeating pattern is (Sp)mRp or Rp(Sp)m, wherein m is 1-50. In some embodiments, the core region of a wing-core-wing motif comprises repeating pattern of internucleotidic linkage stereochemistry, wherein the repeating pattern is (Sp)mRp, wherein m is 1-50. In some embodiments, the core region of a wing-core-wing motif comprises repeating pattern of internucleotidic linkage stereochemistry, wherein the repeating pattern is Rp(Sp)m, wherein m is 1-50. In some embodiments, the core region of a wing-core-wing motif has repeating pattern of internucleotidic linkage stereochemistry, wherein the repeating pattern is (Sp)mRp or Rp(Sp)m, wherein m is 1-50. In some embodiments, the core region of a wing-core-wing motif has repeating pattern of internucleotidic linkage stereochemistry, wherein the repeating pattern is (Sp)mRp, wherein m is 1-50. In some embodiments, the core region of a wing-core-wing motif has repeating pattern of internucleotidic linkage stereochemistry, wherein the repeating pattern is Rp(Sp)m, wherein m is 1-50. In some embodiments, the core region of a wing-core-wing motif has repeating pattern of internucleotidic linkage stereochemistry, wherein the repeating pattern is a motif comprising at least 33% of internucleotidic linkage in the S conformation. In some embodiments, the core region of a wing-core-wing motif has repeating pattern of internucleotidic linkage stereochemistry, wherein the repeating pattern is a motif comprising at least 50% of internucleotidic linkage in the S conformation. In some embodiments, the core region of a wing-core-wing motif has repeating pattern of internucleotidic linkage stereochemistry, wherein the repeating pattern is a motif comprising at least 66% of internucleotidic linkage in the S conformation. In some embodiments, the core region of a wing-core-wing motif has repeating pattern of internucleotidic linkage stereochemistry, wherein the repeating pattern is a repeating triplet motif selected from RpRpSp and SpSpRp. In some embodiments, the core region of a wing-core-wing motif has repeating pattern of internucleotidic linkage stereochemistry, wherein the repeating pattern is a repeating RpRpSp. In some embodiments, the core region of a wing-core-wing motif has repeating pattern of internucleotidic linkage stereochemistry, wherein the repeating pattern is a repeating SpSpRp.

In some embodiments, the present disclosure provides a chirally controlled oligonucleotide composition of an oligonucleotide type whose pattern of backbone chiral centers in the core region comprises (Sp)mRp or Rp(Sp)m. In some embodiments, the present disclosure provides a chirally controlled oligonucleotide composition of an oligonucleotide type whose pattern of backbone chiral centers in the core region comprises Rp(Sp)m. In some embodiments, the present disclosure provides a chirally controlled oligonucleotide composition of an oligonucleotide type whose pattern of backbone chiral centers in the core region comprises (Sp)mRp. In some embodiments, m is 2. In some embodiments, the present disclosure provides a chirally controlled oligonucleotide composition of an oligonucleotide type whose pattern of backbone chiral centers in the core region comprises Rp(Sp)₂. In some embodiments, the present disclosure provides a chirally controlled oligonucleotide composition of an oligonucleotide type whose pattern of backbone chiral centers in the core region comprises (Sp)₂Rp(Sp)₂. In some embodiments, the present disclosure provides a chirally controlled oligonucleotide composition of an oligonucleotide type whose pattern of backbone chiral centers in the core region comprises (Rp)₂Rp(SpP)₂. In some embodiments, the present disclosure provides a chirally controlled oligonucleotide composition of an oligonucleotide type whose pattern of backbone chiral centers in the core region comprises RpSpRp(Sp). In some embodiments, the present disclosure provides a chirally controlled oligonucleotide composition of an oligonucleotide type whose pattern of backbone chiral centers in the core region comprises SpRpRp(Sp)₂. In some embodiments, the present disclosure provides a chirally controlled oligonucleotide composition of an oligonucleotide type whose pattern of backbone chiral centers in the core region comprises (Sp)₂Rp.

In some embodiments, the present disclosure provides a chirally controlled oligonucleotide composition of an oligonucleotide type whose pattern of backbone chiral centers comprises (Sp)mRp or Rp(Sp)m. In some embodiments, the present disclosure provides a chirally controlled oligonucleotide composition of an oligonucleotide type whose pattern of backbone chiral centers comprises Rp(Sp)m. In some embodiments, the present disclosure provides a chirally controlled oligonucleotide composition of an oligonucleotide type whose pattern of backbone chiral centers comprises (Sp)mRp. In some embodiments, m is 2. In some embodiments, the present disclosure provides a chirally controlled oligonucleotide composition of an oligonucleotide type whose pattern of backbone chiral centers comprises Rp(Sp)₂. In some embodiments, the present disclosure provides a chirally controlled oligonucleotide composition of an oligonucleotide type whose pattern of backbone chiral centers comprises (p)₂Rp(Sp)₂. In some embodiments, the present disclosure provides a chirally controlled oligonucleotide composition of an oligonucleotide type whose pattern of backbone chiral centers comprises (Rp)₂Rp(Sp)₂. In some embodiments, the present disclosure provides a chirally controlled oligonucleotide composition of an oligonucleotide type whose pattern of backbone chiral centers comprises RpSpRp(Sp)₂. In some embodiments, the present disclosure provides a chirally controlled oligonucleotide composition of an oligonucleotide type whose pattern of backbone chiral centers comprises SpRpRp(Sp)₂. In some embodiments, the present disclosure provides a chirally controlled oligonucleotide composition of an oligonucleotide type whose pattern of backbone chiral centers comprises (Sp)₂Rp.

As defined herein, m is 1-50. In some embodiments, m is 1. In some embodiments, m is 2-50. In some embodiments, m is 2, 3, 4, 5, 6, 7 or 8. In some embodiments, m is 3, 4, 5, 6, 7 or 8. In some embodiments, m is 4, 5, 6, 7 or 8. In some embodiments, m is 5, 6, 7 or 8. In some embodiments, m is 6, 7 or 8. In some embodiments, m is 7 or 8. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, in is 17. In some embodiments, m is 18. In some embodiments, n is 19. In some embodiments, in is 20. In some embodiments, m is 21. In some embodiments, m is 22. In some embodiments, m is 23. In some embodiments, m is 24. In some embodiments, m is 25. In some embodiments, m is greater than 25.

In some embodiments, a repeating pattern is (Sp)m(Rp)n, wherein n is 1-10, and m is independently as defined above and described herein. In some embodiments, the present disclosure provides a chirally controlled oligonucleotide composition of an oligonucleotide type whose pattern of backbone chiral centers comprises (Sp)m(Rp)n. In some embodiments, the present disclosure provides a chirally controlled oligonucleotide composition of an oligonucleotide type whose pattern of backbone chiral centers in the core region comprises (Sp)m(Rp)n. In some embodiments, a repeating pattern is (Rp)n(Sp)m, wherein n is 1-10, and m is independently as defined above and described herein. In some embodiments, the present disclosure provides a chirally controlled oligonucleotide composition of an oligonucleotide type whose pattern of backbone chiral centers comprises (Rp)n(Sp)m. In some embodiments, the present disclosure provides a chirally controlled oligonucleotide composition of an oligonucleotide type whose pattern of backbone chiral centers in the core region comprises (Rp)n(Sp)m. In some embodiments, (Rp)n(Sp)m is (Rp)(Sp)₂. In some embodiments, (Sp)n(Rp)m is (Sp)₂(Rp).

In some embodiments, the present disclosure provides a chirally controlled oligonucleotide composition of an oligonucleotide type whose pattern of backbone chiral centers comprises (Sp)m(Rp)n(Sp)t. In some embodiments, a repeating pattern is (Sp)m(Rp)n(Sp)t, wherein n is 1-10, t is 1-50, and m is as defined above and described herein. In some embodiments, the present disclosure provides a chirally controlled oligonucleotide composition of an oligonucleotide type whose pattern of backbone chiral centers in the core region comprises (Sp)m(Rp)n(Sp)t. In some embodiments, a repeating pattern is (Sp)t(Rp)n(Sp)m, wherein n is 1-10, t is 1-50, and m is as defined above and described herein. In some embodiments, the present disclosure provides a chirally controlled oligonucleotide composition of an oligonucleotide type whose pattern of backbone chiral centers comprises (Sp)t(Rp)n(Sp)m. In some embodiments, the present disclosure provides a chirally controlled oligonucleotide composition of an oligonucleotide type whose pattern of backbone chiral centers in the core region comprises (Sp)t(Rp)n(Sp)m.

In some embodiments, a repeating pattern is (Np)t(Rp)n(Sp)m, wherein n is 1-10, t is 1-50, Np is independently Rp or Sp, and m is as defined above and described herein. In some embodiments, the present disclosure provides a chirally controlled oligonucleotide composition of an oligonucleotide type whose pattern of backbone chiral centers comprises (Np)t(Rp)n(Sp)m. In some embodiments, the present disclosure provides a chirally controlled oligonucleotide composition of an oligonucleotide type whose pattern of backbone chiral centers in the core region comprises (Np)t(Rp)n(Sp)m. In some embodiments, a repeating pattern is (Np)m(Rp)n(Sp)t, wherein n is 1-10, t is 1-50, Np is independently Rp or Sp, and m is as defined above and described herein. In some embodiments, the present disclosure provides a chirally controlled oligonucleotide composition of an oligonucleotide type whose pattern of backbone chiral centers comprises (Np)m(Rp)n(Sp)t. In some embodiments, the present disclosure provides a chirally controlled oligonucleotide composition of an oligonucleotide type whose pattern of backbone chiral centers in the core region comprises (Np)m(Rp)n(Sp)t. In some embodiments, Np is Rp. In some embodiments, Np is Sp. In some embodiments, all Np are the same. In some embodiments, all Np are Sp. In some embodiments, at least one Np is different from the other Np. In some embodiments, t is 2.

As defined herein, n is 1-10. In some embodiments, n is 1, 2, 3, 4, 5, 6, 7 or 8. In some embodiments, n is 1. In some embodiments, n is 2, 3, 4, 5, 6, 7 or 8. In some embodiments, n is 3, 4, 5, 6, 7 or 8. In some embodiments, n is 4, 5, 6, 7 or 8. In some embodiments, n is 5, 6, 7 or 8. In some embodiments, n is 6, 7 or 8. In some embodiments, n is 7 or 8. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

As defined herein, t is 1-50. In some embodiments, t is 1. In some embodiments, t is 2-50. In some embodiments, t is 2, 3, 4, 5, 6, 7 or 8. In some embodiments, t is 3, 4, 5, 6, 7 or 8. In some embodiments, t is 4, 5, 6, 7 or 8. In some embodiments, t is 5, 6, 7 or 8. In some embodiments, t is 6, 7 or 8. In some embodiments, t is 7 or 8. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, t is 11. In some embodiments, t is 12. In some embodiments, t is 13. In some embodiments, t is 14. In some embodiments, t is 15. In some embodiments, t is 16. In some embodiments, t is 17. In some embodiments, t is 18. In some embodiments, t is 19. In some embodiments, t is 20. In some embodiments, t is 21. In some embodiments, t is 22. In some embodiments, t is 23. In some embodiments, t is 24. In some embodiments, t is 25. In some embodiments, t is greater than 25.

In some embodiments, at least one of m and t is greater than 2. In some embodiments, at least one of m and t is greater than 3. In some embodiments, at least one of m and t is greater than 4. In some embodiments, at least one of m and t is greater than 5. In some embodiments, at least one of m and t is greater than 6. In some embodiments, at least one of n and t is greater than 7. In some embodiments, at least one of m and t is greater than 8. In some embodiments, at least one of m and t is greater than 9. In some embodiments, at least one of in and t is greater than 10. In some embodiments, at least one of m and t is greater than 11. In some embodiments, at least one of m and t is greater than 12. In some embodiments, at least one of m and t is greater than 13. In some embodiments, at least one of m and t is greater than 14. In some embodiments, at least one of m and t is greater than 15. In some embodiments, at least one of m and t is greater than 16. In some embodiments, at least one of m and t is greater than 17. In some embodiments, at least one of m and t is greater than 18. In some embodiments, at least one of m and t is greater than 19. In some embodiments, at least one of m and t is greater than 20. In some embodiments, at least one of m and t is greater than 21. In some embodiments, at least one of m and t is greater than 22. In some embodiments, at least one of m and t is greater than 23. In some embodiments, at least one of m and t is greater than 24. In some embodiments, at least one of m and t is greater than 25.

In some embodiments, each one of m and t is greater than 2. In some embodiments, each one of m and t is greater than 3. In some embodiments, each one of m and t is greater than 4. In some embodiments, each one of m and t is greater than 5. In some embodiments, each one of m and t is greater than 6. In some embodiments, each one of m and t is greater than 7. In some embodiments, each one of m and t is greater than 8. In some embodiments, each one of m and t is greater than 9. In some embodiments, each one of m and t is greater than 10. In some embodiments, each one of m and t is greater than 11. In some embodiments, each one of in and t is greater than 12. In some embodiments, each one of m and t is greater than 13. In some embodiments, each one of m and t is greater than 14. In some embodiments, each one of m and t is greater than 15 In some embodiments, each one of m and t is greater than 16. In some embodiments, each one of m and t is greater than 17. In some embodiments, each one of m and t is greater than 18. In some embodiments, each one of m and t is greater than 19. In some embodiments, each one of m and t is greater than 20.

In some embodiments, the sum of m and t is greater than 3. In some embodiments, the sum of m and t is greater than 4. In some embodiments, the sum of in and t is greater than 5. In some embodiments, the sum of m and t is greater than 6. In some embodiments, the sum of in and t is greater than 7. In some embodiments, the sum of m and t is greater than 8. In some embodiments, the sum of m and t is greater than 9. In some embodiments, the sum of m and t is greater than 10. In some embodiments, the sum of m and t is greater than 11. In some embodiments, the sum of m and t is greater than 12. In some embodiments, the sum of m and t is greater than 13. In some embodiments, the sum of m and t is greater than 14. In some embodiments, the sum of m and t is greater than 15. In some embodiments, the sum of m and t is greater than 16. In some embodiments, the sum of m and t is greater than 17. In some embodiments, the sum of in and t is greater than 18. In some embodiments, the sum of m and t is greater than 19. In some embodiments, the sum of m and t is greater than 20. In some embodiments, the sum of m and t is greater than 21. In some embodiments, the sum of m and t is greater than 22. In some embodiments, the sum of m and t is greater than 23. In some embodiments, the sum of m and t is greater than 24. In some embodiments, the sum of m and t is greater than 25.

In some embodiments, n is 1, and at least one of m and t is greater than 1. In some embodiments, n is 1 and each of m and t is independently greater than 1. In some embodiments, m>n and t>n. In some embodiments, (Sp)m(Rp)n(Sp)t is (Sp)₂Rp(Sp)₂. In some embodiments, (Sp)t(Rp)n(Sp)m is (Sp)₂Rp(Sp)₂. In some embodiments, (Sp)t(Rp)n(Sp)m is SpRp(Sp)₂. In some embodiments, (Np)t(Rp)n(Sp)m is (Np)tRp(Sp)m. In some embodiments, (Np)t(Rp)n(Sp)m is (Np)₂Rp(Sp)m. In some embodiments, (Np)t(Rp)n(Sp)m is (Rp)₂Rp(Sp)m. In some embodiments, (Np)t(Rp)n(Sp)m is (Sp)₂Rp(Sp)m. In some embodiments, (Np)t(Rp)n(Sp)m is RpSpRp(Sp)m. In some embodiments, (Np)t(Rp)n(Sp)m is SpRpRp(Sp)m.

In some embodiments, (Sp)t(Rp)n(Sp)m is SpRpSpSp. In some embodiments, (Sp)t(Rp)n(Sp)m is (Sp)₂Rp(Sp)₂. In some embodiments, (Sp)t(Rp)n(Sp)m is (Sp)₃Rp(Sp)₃. In some embodiments, (Sp)t(Rp)n(Sp)m is (Sp)₄Rp(Sp)₄. In some embodiments, (Sp)t(Rp)n(Sp)m is (Sp)tRp(Sp)₅. In some embodiments, (Sp)t(Rp)n(Sp)m is SpRp(Sp)₅. In some embodiments, (Sp)t(Rp)n(Sp)m is (Sp)₂Rp(Sp)₅. In some embodiments, (Sp)t(Rp)n(Sp)m is (Sp)₃Rp(Sp)₅. In some embodiments, (Sp)t(Rp)n(Sp)m is (Sp)₄Rp(Sp)₅. In some embodiments, (Sp)t(Rp)n(Sp)m is (Sp)₅Rp(Sp)₅.

In some embodiments, (Sp)m(Rp)n(Sp)t is (Sp)₂Rp(Sp)₂. In some embodiments, (Sp)m(Rp)n(Sp)t is (Sp)₃Rp(Sp)₃. In some embodiments, (Sp)m(Rp)n(Sp)t is (Sp)₄Rp(Sp)₄. In some embodiments, (Sp)m(Rp)n(Sp)t is (Sp)mRp(Sp)₅. In some embodiments, (Sp)m(Rp)n(Sp)t is (Sp)₂Rp(Sp)₅. In some embodiments, (Sp)m(Rp)n(Sp)t is (Sp)₃Rp(Sp)₅. In some embodiments, (Sp)m(Rp)n(Sp)t is (Sp)₄Rp(Sp)₅. In some embodiments, (Sp)m(Rp)n(Sp)t is (Sp)₅Rp(Sp)₅.

In some embodiments, a core region comprises at least one Rp internucleotidic linkage. In some embodiments, a core region of a wing-core-wing motif comprises at least one Rp internucleotidic linkage. In some embodiments, a core region comprises at least one Rp phosphorothioate internucleotidic linkage. In some embodiments, a core region of a wing-core-wing motif comprises at least one Rp phosphorothioate internucleotidic linkage. In some embodiments, a core region of a wing-core-wing motif comprises only one Rp phosphorothioate internucleotidic linkage. In some embodiments, a core region motif comprises at least two Rp internucleotidic linkages. In some embodiments, a core region of a wing-core-wing motif comprises at least two Rp internucleotidic linkages. In some embodiments, a core region of a wing-core-wing motif comprises at least two Rp phosphorothioate internucleotidic linkages. In some embodiments, a core region comprises at least three Rp internucleotidic linkages. In some embodiments, a core region of a wing-core-wing motif comprises at least three Rp internucleotidic linkages. In some embodiments, a core region comprises at least three Rp phosphorothioate internucleotidic linkages. In some embodiments, a core region of a wing-core-wing motif comprises at least three Rp phosphorothioate internucleotidic linkages. In some embodiments, a core region comprises at least 4, 5, 6, 7, 8, 9, or 10 Rp internucleotidic linkages. In some embodiments, a core region of a wing-core-wing motif comprises at least 4, 5, 6, 7, 8, 9, or 10 Rp internucleotidic linkages. In some embodiments, a core region comprises at least 4, 5, 6, 7, 8, 9, or 10 Rp phosphorothioate internucleotidic linkages. In some embodiments, a core region of a wing-core-wing motif comprises at least 4, 5, 6, 7, 8, 9, or 10 Rp phosphorothioate internucleotidic linkages.

In some embodiments, a wing region comprises 2′-modifications of sugar moieties that differ from a core region. In some embodiments, a wing region comprises the same type of 2′-modifications that differ from a core region. In some embodiments, a wing region comprises 2′-F which is absent from a core region. In some embodiments, a wing region comprises a pattern of 2′-F which is absent from a core region. In some embodiments, a wing region comprises a level of 2′-F which differs from a core region. In some embodiments, a level is absolute as measured by the number of 2′-F modifications. In some embodiments, a level is relative as measured by the percentage of 2′-F modifications. In some embodiments, a wing region differs from a core region in that it contains less of a 2′-modification presented in a core region, as measured by the number and/or percentage of such 2′-modifications. In some embodiments, a wing region contains less of a 2′-OR¹ modification in a core region. In some embodiments, a wing region contains less of a 2′-OMe modification in a core region. In some embodiments, a wing region differs from a core region in that it contains less of unmodified sugar moieties presented in a core region, as measured by the number and/or percentage of such 2′-modifications.

In some embodiments, provided oligonucleotides comprise two or more wing regions and a core region, for example, provided oligonucleotides may comprise a wing-core-wing structure. In some embodiments, each wing region comprises 2′-modifications of sugar moieties that differ from a core region. In some embodiments, each wing region comprises the same type of 2′-modifications that differ from a core region. In some embodiments, each wing region comprises 2′-F which is absent from a core region. In some embodiments, each wing region comprises a pattern of 2′-F which is absent from a core region. In some embodiments, each wing region comprises a level of 2′-F which differs from a core region. In some embodiments, a level is absolute as measured by the number of 2′-F modifications. In some embodiments, a level is relative as measured by the percentage of 2′-F modifications. In some embodiments, each wing region differs from a core region in that it contains less of a 2′-modification presented in a core region, as measured by the number and/or percentage of such 2′-modifications. In some embodiments, each wing region contains less of a 2′-OR¹ modification in a core region. In some embodiments, each wing region contains less of a 2′-OMe modification in a core region. In some embodiments, each wing region differs from a core region in that it contains less of unmodified sugar moieties presented in a core region, as measured by the number and/or percentage of such 2′-modifications.

In certain embodiments, a wing-core-wing motif is a 5-10-5 motif wherein the residues at each wing region are 2′-modified residues. In certain embodiments, a wing-core-wing motif is a 5-10-5 motif wherein the residues at each wing region are 2′-OR¹-modified residues. In certain embodiments, a wing-core-wing motif is a 5-10-5 motif wherein the residues at each wing region are 2′-MOE-modified residues. In certain embodiments, a wing-core-wing motif is a 5-10-5 motif wherein the residues at each wing region are 2′-OMe-modified residues. In certain embodiments, a wing-core-wing motif is a 5-10-5 motif wherein the residues at each wing region are 2′-F-modified residues. In certain embodiments, a wing-core-wing motif is a 5-10-5 motif wherein the residues in the core region are 2′-deoxyribonucleoside residues. In certain embodiments, a wing-core-wing motif is a 5-10-5 motif, wherein all internucleotidic linkages are phosphorothioate linkages. In certain embodiments, a wing-core-wing motif is a 5-10-5 motif, wherein all internucleotidic linkages are chiral phosphorothioate linkages. In certain embodiments, a wing-core-wing motif is a 5-10-5 motif wherein the residues at each wing region are 2′-modified residues, the residues in the core region are 2′-deoxyribonucleoside residues, and all internucleotidic linkages in the core region are chiral phosphorothioate linkages. In certain embodiments, a wing-core-wing motif is a 5-10-5 motif wherein the residues at each wing region are 2′-OR¹-modified residues, the residues in the core region are 2′-deoxyribonucleoside residues, and all internucleotidic linkages in the core region are chiral phosphorothioate linkages. In certain embodiments, a wing-core-wing motif is a 5-10-5 motif wherein the residues at each wing region are 2′-MOE-modified residues, the residues in the core region are 2′-deoxyribonucleoside residues, and all internucleotidic linkages in the core region are chiral phosphorothioate linkages. In certain embodiments, a wing-core-wing motif is a 5-10-5 motif wherein the residues at each wing region are 2′-OMe-modified residues, the residues in the core region are 2′-deoxyribonucleoside residues, and all internucleotidic linkages in the core region are chiral phosphorothioate linkages.

In some embodiments, residues at the “X” wing region are not 2′-MOE-modified residues. In certain embodiments, a wing-core motif is a motif wherein the residues at the “X” wing region are not 2′-MOE-modified residues. In certain embodiments, a core-wing motif is a motif wherein the residues at the “X” wing region are not 2′-MOE-modified residues. In certain embodiments, a wing-core-wing motif is a motif wherein the residues at each “X” wing region are not 2′-MOE-modified residues. In certain embodiments, a wing-core-wing motif is a 5-10-5 motif wherein the residues at each “X” wing region are not 2′-MOE-modified residues. In certain embodiments, a wing-core-wing motif is a 5-10-5 motif wherein the residues in the core “Y” region are 2′-deoxyribonucleoside residues. In certain embodiments, a wing-core-wing motif is a 5-10-5 motif, wherein all internucleotidic linkages are phosphorothioate internucleotidic linkages. In certain embodiments, a wing-core-wing motif is a 5-10-5 motif, wherein all internucleotidic linkages are chiral phosphorothioate internucleotidic linkages. In certain embodiments, a wing-core-wing motif is a 5-10-5 motif wherein the residues at each “X” wing region are not 2′-MOE-modified residues, the residues in the core “Y” region are 2′-deoxyribonucleoside, and all internucleotidic linkages are chiral phosphorothioate internucleotidic linkages.

In some embodiments, a chiral, modified phosphate linkage is a chiral phosphorothioate linkage, i.e., phosphorothioate internucleotidic linkage. In some embodiments, a core region comprises at least 5%, 10%, 15%, 20%, 25%, 30%, 35%, 40%, 45%, 50%, 55%, 60%, 65%, 70%, 75%, 80%, 85%, 90%, 95%, or 100% chiral phosphorothioate internucleotidic linkages. In some embodiments, all chiral, modified phosphate linkages are chiral phosphorothioate internucleotidic linkages. In some embodiments, at least about 10%, 20%, 30%, 40%, 50%, 60%, 70%, 80%, or 90% chiral phosphorothioate internucleotidic linkages of a core region are of the Sp conformation. In some embodiments, at least about 10% chiral phosphorothioate internucleotidic linkages of a core region are of the Sp conformation. In some embodiments, at least about 20% chiral phosphorothioate internucleotidic linkages of a core region are of the Sp conformation. In some embodiments, at least about 30% chiral phosphorothioate internucleotidic linkages of a core region are of the Sp conformation. In some embodiments, at least about 40% chiral phosphorothioate internucleotidic linkages of a core region are of the Sp conformation. In some embodiments, at least about 50% chiral phosphorothioate internucleotidic linkages of a core region are of the Sp conformation. In some embodiments, at least about 60% chiral phosphorothioate internucleotidic linkages of a core region are of the Sp conformation. In some embodiments, at least about 70% chiral phosphorothioate internucleotidic linkages of a core region are of the Sp conformation. In some embodiments, at least about 80% chiral phosphorothioate internucleotidic linkages of a core region are of the Sp conformation. In some embodiments, at least about 90% chiral phosphorothioate internucleotidic linkages of a core region are of the Sp conformation. In some embodiments, at least about 95% chiral phosphorothioate internucleotidic linkages of a core region are of the Sp conformation.

In some embodiments, at least about 10%, 20%, 30%, 40%, 50%, 60%, 70%, 80%, or 90% chiral phosphorothioate internucleotidic linkages of a core region are of the Rp conformation. In some embodiments, at least about 10% chiral phosphorothioate internucleotidic linkages of a core region are of the Rp conformation. In some embodiments, at least about 20% chiral phosphorothioate internucleotidic linkages of a core region are of the Rp conformation. In some embodiments, at least about 30% chiral phosphorothioate internucleotidic linkages of a core region are of the Rp conformation. In some embodiments, at least about 40% chiral phosphorothioate internucleotidic linkages of a core region are of the Rp conformation. In some embodiments, at least about 50% chiral phosphorothioate internucleotidic linkages of a core region are of the Rp conformation. In some embodiments, at least about 60% chiral phosphorothioate internucleotidic linkages of a core region are of the Rp conformation. In some embodiments, at least about 70% chiral phosphorothioate internucleotidic linkages of a core region are of the Rp conformation. In some embodiments, at least about 80% chiral phosphorothioate internucleotidic linkages of a core region are of the Rp conformation. In some embodiments, at least about 90% chiral phosphorothioate internucleotidic linkages of a core region are of the Rp conformation. In some embodiments, at least about 95% chiral phosphorothioate internucleotidic linkages of a core region are of the Rp conformation.

In some embodiments, less than about 10%, 20%, 30%, 40%, 50%, 60%, 70%, 80%, or 90% chiral phosphorothioate internucleotidic linkages of a core region are of the Rp conformation. In some embodiments, less than about 10% chiral phosphorothioate internucleotidic linkages of a core region are of the Rp conformation. In some embodiments, less than about 20% chiral phosphorothioate internucleotidic linkages of a core region are of the Rp conformation. In some embodiments, less than about 30% chiral phosphorothioate internucleotidic linkages of a core region are of the Rp conformation. In some embodiments, less than about 40% chiral phosphorothioate internucleotidic linkages of a core region are of the Rp conformation. In some embodiments, less than about 50% chiral phosphorothioate internucleotidic linkages of a core region are of the Rp conformation. In some embodiments, less than about 60% chiral phosphorothioate internucleotidic linkages of a core region are of the Rp conformation. In some embodiments, less than about 70% chiral phosphorothioate internucleotidic linkages of a core region are of the Rp conformation. In some embodiments, less than about 80% chiral phosphorothioate internucleotidic linkages of a core region are of the Rp conformation. In some embodiments, less than about 90% chiral phosphorothioate internucleotidic linkages of a core region are of the Rp conformation. In some embodiments, less than about 95% chiral phosphorothioate internucleotidic linkages of a core region are of the Rp conformation. In some embodiments, a core region has only one Rp chiral phosphorothioate internucleotidic linkages. In some embodiments, a core region has only one Rp chiral phosphorothioate internucleotidic linkages, wherein all internucleotide linkages are chiral phosphorothioate internucleotidic linkages.

In some embodiments, provided oligonucleotides are blockmers. In some embodiments, provided oligonucleotide are altmers. In some embodiments, provided oligonucleotides are altmers comprising alternating blocks. In some embodiments, a blockmer or an altmer can be defined by chemical modifications (including presence or absence), e.g., base modifications, sugar modification, internucleotidic linkage modifications, stereochemistry, etc. Example chemical modifications, stereochemistry and patterns thereof for a block and/or an alternating unit include but are not limited to those described in this disclosure, such as those described for a wing, a core, an oligonucleotide, etc. In some embodiments, a blockmer comprises a pattern of ..SS..RR..SS..RR.. In some embodiments, an altmer comprises a pattern of SRSRSRSR.

In some embodiments, a pattern of backbone chiral center, e.g., of a wing, a core, a block, comprises one or more (Rp)p(Sp)x(Rp)q(Sp)y, wherein each of p, x, q, y is independently 0-50, p+q>0, and x+y>0.

In some embodiments, a provided pattern of backbone chiral centers comprises repeating (Sp)m(Rp)n, (Rp)n(Sp)m, (Np)t(Rp)n(Sp)m, or (Sp)t(Rp)n(Sp)m units. In some embodiments, a repeating unit is (Sp)m(Rp)n. In some embodiments, a repeating unit is SpRp. In some embodiments, a repeating unit is SpSpRp. In some embodiments, a repeating unit is SpRpRp. In some embodiments, a repeating unit is RpRpSp. In some embodiments, a repeating unit is (Rp)n(Sp)m. In some embodiments, a repeating unit is (Np)t(Rp)n(Sp)m. In some embodiments, a repeating unit is (Sp)t(Rp)n(Sp)m.

In some embodiments, a provided pattern of backbone chiral centers comprises (Rp/Sp)x-(All Rp or All Sp)-(Rp/Sp)y. In some embodiments, a provided pattern of backbone chiral centers comprises (Rp/Sp)-(All Rp or All Sp)-(Rp/Sp). In some embodiments, a provided pattern of backbone chiral centers comprises (Rp)x-(All Sp)-(Rp)y. In some embodiments, a provided pattern of backbone chiral centers comprises (Rp)-(All Sp)-(Rp). In some embodiments, a provided pattern of backbone chiral centers comprises (Sp)x-(All Rp)-(Sp)y. In some embodiments, a provided pattern of backbone chiral centers comprises (Sp)-(All Rp)-(Sp). In some embodiments, a provided pattern of backbone chiral centers comprises (Rp/Sp)x-(repeating (Sp)m(Rp)n)-(Rp/Sp)y. In some embodiments, a provided pattern of backbone chiral centers comprises (Rp/Sp)-(repeating (Sp)m(Rp)n)-(Rp/Sp). In some embodiments, a provided pattern of backbone chiral centers comprises (Rp/Sp)x-(repeating SpSpRp)-(Rp/Sp)y. In some embodiments, a provided pattern of backbone chiral centers comprises (Rp/Sp)-(repeating SpSpRp)-(Rp/Sp).

In some embodiments, a provided pattern of backbone chiral centers is (Rp/Sp)x-(All Rp or All Sp)-(Rp/Sp)y. In some embodiments, a provided pattern of backbone chiral centers is (Rp/Sp)-(All Rp or All Sp)-(Rp/Sp). In some embodiments, a provided pattern of backbone chiral centers is (Rp)x-(All Sp)-(Rp)y. In some embodiments, a provided pattern of backbone chiral centers is (Rp)-(All Sp)-(Rp). In some embodiments, a provided pattern of backbone chiral centers is (Sp)x-(All Rp)-(Sp)y. In some embodiments, a provided pattern of backbone chiral centers is (Sp)-(All Rp)-(Sp). In some embodiments, a provided pattern of backbone chiral centers is (Rp/Sp)x-(repeating (Sp)m(Rp)n)-(Rp/Sp)y. In some embodiments, a provided pattern of backbone chiral centers is (Rp/Sp)-(repeating (Sp)m(Rp)n)-(Rp/Sp). In some embodiments, a provided pattern of backbone chiral centers is (Rp/Sp)x-(repeating SpSpRp)-(Rp/Sp)y. In some embodiments, a provided pattern of backbone chiral centers is (Rp/Sp)-(repeating SpSpRp)-(Rp/Sp).

A person of ordinary skill in the art understands that various regions of a target transcript can be targeted by provided compositions and methods. In some embodiments, a base sequence of provided oligonucleotides comprises an intron sequence. In some embodiments, a base sequence of provided oligonucleotides comprises an exon sequence. In some embodiments, a base sequence of provided oligonucleotides comprises an intron and an exon sequence. In some embodiments, a base sequence of provided oligonucleotides comprises a sequence spanning a splicing site. In some embodiments, a base sequence of provided oligonucleotides comprises a sequence found in or comprising a 5′ splice site, a branch point sequence (BPS), a polypyrimidine tact (py tact), a 3′ splice site, an intronic splicing silencer (ISS), an exonic splicing silencer (ESS), an intronic splicing enhancer (ISE), and/or an exonic splicing enhancer. In some embodiments, a base sequence of provided oligonucleotides is an intron sequence. In some embodiments, a base sequence of provided oligonucleotides is an exon sequence. In some embodiments, a base sequence of provided oligonucleotides is a sequence spanning a splicing site. In some embodiments, a base sequence of provided oligonucleotides is a sequence found in or comprising a 5′ splice site, a branch point sequence (BPS), a polypyrimidine tact (py tact), a 3′ splice site, an intronic splicing silencer (ISS), an exonic splicing silencer (ESS), an intronic splicing enhancer (ISE), and/or an exonic splicing enhancer. In some embodiments, a base sequence of provided oligonucleotides is a sequence found in a branch point sequence (BPS), a polypyrimidine tact (py tact), an intronic splicing silencer (ISS), an exonic splicing silencer (ESS), an intronic splicing enhancer (ISE), and/or an exonic splicing enhancer.

As understood by a person having ordinary skill in the art, provided oligonucleotides and compositions, among other things, can target a great number of nucleic acid polymers. For instance, in some embodiments, provided oligonucleotides and compositions may target a transcript of a nucleic acid sequence, wherein a common base sequence of oligonucleotides (e.g., a base sequence of an oligonucleotide type) comprises or is a sequence complementary to a sequence of the transcript. In some embodiments, a common base sequence comprises a sequence complimentary to a sequence of a target. In some embodiments, a common base sequence is a sequence complimentary to a sequence of a target. In some embodiments, a common base sequence comprises or is a sequence 100% complimentary to a sequence of a target. In some embodiments, a common base sequence comprises a sequence 100% complimentary to a sequence of a target. In some embodiments, a common base sequence is a sequence 100% complimentary to a sequence of a target. In some embodiments, a common base sequence in a core comprises or is a sequence complimentary to a sequence of a target. In some embodiments, a common base sequence in a core comprises a sequence complimentary to a sequence of a target. In some embodiments, a common base sequence in a core is a sequence % complimentary to a sequence of a target. In some embodiments, a common base sequence in a core comprises or is a sequence 100% complimentary to a sequence of a target. In some embodiments, a common base sequence in a core comprises a sequence 100% complimentary to a sequence of a target. In some embodiments, a common base sequence in a core is a sequence 100% complimentary to a sequence of a target.

In some embodiments, as described in this disclosure, provided oligonucleotides and compositions may provide new cleavage patterns, higher cleavage rate, higher cleavage degree, higher cleavage selectivity, etc. In some embodiments, provided compositions can selectively suppress (e.g., cleave) a transcript from a target nucleic acid sequence which has one or more similar sequences exist within a subject or a population, each of the target and its similar sequences contains a specific nucleotidic characteristic sequence element that defines the target sequence relative to the similar sequences. In some embodiments, for example, a target sequence is a wild-type allele or copy of a gene, and a similar sequence is a sequence has very similar base sequence, e.g., a sequence having SNP, mutations, etc.

In some embodiments, a similar sequence has greater than 60%, 65%, 70%, 75%, 80%, 85%, 90%, 91%, 92%, 93%, 94%, 95%, 96%, 97%, 98% or 99% sequence homology with a target sequence. In some embodiments, a target sequence is a disease-causing copy of a nucleic acid sequence comprising one or more mutations and/or SNPs, and a similar sequence is a copy not causing the disease (wild type). In some embodiments, a target sequence comprises a mutation, wherein a similar sequence is the corresponding wild-type sequence. In some embodiments, a target sequence is a mutant allele, while a similar sequence is a wild-type allele. In some embodiments, a target sequence comprises an SNP that is associated with a disease-causing allele, while a similar sequence comprises the same SNP that is not associates with the disease-causing allele. In some embodiments, the region of a target sequence that is complementary to a common base sequence of a provided oligonucleotide composition has greater than 60%, 65%, 70%, 75%, 80%, 85%, 90%, 91%, 92%, 93%, 94%, 95%, 96%, 97%, 98% or 99% sequence homology with the corresponding region of a similar sequence. In some embodiments, the region of a target sequence that is complementary to a common base sequence of a provided oligonucleotide composition differs from the corresponding region of a similar sequence at less than 5, less than 4, less than 3, less than 2, or only 1 base pairs. In some embodiments, the region of a target sequence that is complementary to a common base sequence of a provided oligonucleotide composition differs from the corresponding region of a similar sequence only at a mutation site or SNP site. In some embodiments, the region of a target sequence that is complementary to a common base sequence of a provided oligonucleotide composition differs from the corresponding region of a similar sequence only at a mutation site. In some embodiments, the region of a target sequence that is complementary to a common base sequence of a provided oligonucleotide composition differs from the corresponding region of a similar sequence only at an SNP site.

In some embodiments, a common base sequence comprises or is a sequence complementary to a characteristic sequence element. In some embodiments, a common base sequence comprises a sequence complementary to a characteristic sequence element. In some embodiments, a common base sequence is a sequence complementary to a characteristic sequence element. In some embodiments, a common base sequence comprises or is a sequence 100% complementary to a characteristic sequence element. In some embodiments, a common base sequence comprises a sequence 100% complementary to a characteristic sequence element. In some embodiments, a common base sequence is a sequence 100% complementary to a characteristic sequence element. In some embodiments, a common base sequence in a core comprises or is a sequence complementary to a characteristic sequence element. In some embodiments, a common base sequence in a core comprises a sequence complementary to a characteristic sequence element. In some embodiments, a common base sequence in a core is a sequence complementary to a characteristic sequence element. In some embodiments, a common base sequence in a core comprises or is a sequence 100% complementary to a characteristic sequence element. In some embodiments, a common base sequence in a core comprises a sequence 100% complementary to a characteristic sequence element. In some embodiments, a common base sequence in a core is a sequence 100% complementary to a characteristic sequence element.

Among other things, the present disclosure recognizes that a base sequence may have impact on oligonucleotide properties. In some embodiments, a base sequence may have impact on cleavage pattern of a target when oligonucleotides having the base sequence are utilized for suppressing a target, e.g., through a pathway involving RNase H: for example, FIG. 33 demonstrates that structurally similar (all phosphorothioate linkages, all stereorandom) oligonucleotides have different sequences may have different cleavage patterns. In some embodiments, a common base sequence of a non-stereorandom oligonucleotide compositions (e.g., certain oligonucleotide compositions provided in the present disclosure) is a base sequence that when applied to a DNA oligonucleotide composition or a stereorandom all-phosphorothioate oligonucleotide composition, cleavage pattern of the DNA (DNA cleavage pattern) and/or the stereorandom all-phosphorothioate (stereorandom cleavage pattern) composition has a cleavage site within or in the vicinity of a characteristic sequence element. In some embodiments, a cleavage site within or in the vicinity is within a sequence complementary to a core region of a common sequence. In some embodiments, a cleavage site within or in the vicinity is within a sequence 100% complementary to a core region of a common sequence.

In some embodiments, a common base sequence is a base sequence that has a cleavage site within or in the vicinity of a characteristic sequence element in its DNA cleavage pattern. In some embodiments, a common base sequence is a base sequence that has a cleavage site within a characteristic sequence element in its DNA cleavage pattern. In some embodiments, a common base sequence is a base sequence that has a cleavage site in the vicinity of a characteristic sequence element in its DNA cleavage pattern. In some embodiments, a common base sequence is a base sequence that has a cleavage site in the vicinity of a mutation or SNP of a characteristic sequence element in its DNA cleavage pattern. In some embodiments, a common base sequence is a base sequence that has a cleavage site in the vicinity of a mutation in its DNA cleavage pattern. In some embodiments, a common base sequence is a base sequence that has a cleavage site in the vicinity of an SNP in its DNA cleavage pattern.

In some embodiments, a common base sequence is a base sequence that has a cleavage site within or in the vicinity of a characteristic sequence element in its stereorandom cleavage pattern. In some embodiments, a common base sequence is a base sequence that has a cleavage site within a characteristic sequence element in its stereorandom cleavage pattern. In some embodiments, a common base sequence is a base sequence that has a cleavage site in the vicinity of a characteristic sequence element in its stereorandom cleavage pattern. In some embodiments, a common base sequence is a base sequence that has a cleavage site in the vicinity of a mutation or SNP of a characteristic sequence element in its stereorandom cleavage pattern. In some embodiments, a common base sequence is a base sequence that has a cleavage site in the vicinity of a mutation in its stereorandom cleavage pattern. In some embodiments, a common base sequence is a base sequence that has a cleavage site in the vicinity of an SNP in its stereorandom cleavage pattern.

In some embodiments, a common base sequence comprises or is a sequence complementary to a nucleic acid sequence. In some embodiments, a common base sequence comprises or is a sequence 100% complementary to a nucleic acid sequence. In some embodiments, a common base sequence comprises or is a sequence complementary to a disease-causing nucleic acid sequence. In some embodiments, a common base sequence comprises or is a sequence 100% complementary to a disease-causing nucleic acid sequence. In some embodiments, a common base sequence comprises or is a sequence complementary to a characteristic sequence element of disease-causing nucleic acid sequence, which characteristic sequences differentiate a disease-causing nucleic acid sequence from a non-diseasing-causing nucleic acid sequence. In some embodiments, a common base sequence comprises or is a sequence 100% complementary to a characteristic sequence element of disease-causing nucleic acid sequence, which characteristic sequences differentiate a disease-causing nucleic acid sequence from a non-diseasing-causing nucleic acid sequence. In some embodiments, a common base sequence comprises or is a sequence complementary to a disease-associated nucleic acid sequence. In some embodiments, a common base sequence comprises or is a sequence 100% complementary to a disease-associated nucleic acid sequence. In some embodiments, a common base sequence comprises or is a sequence complementary to a characteristic sequence element of disease-associated nucleic acid sequence, which characteristic sequences differentiate a disease-associated nucleic acid sequence from a non-diseasing-associated nucleic acid sequence. In some embodiments, a common base sequence comprises or is a sequence 100% complementary to a characteristic sequence element of disease-associated nucleic acid sequence, which characteristic sequences differentiate a disease-associated nucleic acid sequence from a non-diseasing-associated nucleic acid sequence.

In some embodiments, a common base sequence comprises or is a sequence complementary to a gene. In some embodiments, a common base sequence comprises or is a sequence 100% complementary to a gene. In some embodiments, a common base sequence comprises or is a sequence complementary to a characteristic sequence element of a gene, which characteristic sequences differentiate the gene from a similar sequence sharing homology with the gene. In some embodiments, a common base sequence comprises or is a sequence 100% complementary to a characteristic sequence element of a gene, which characteristic sequences differentiate the gene from a similar sequence sharing homology with the gene. In some embodiments, a common base sequence comprises or is a sequence complementary to characteristic sequence element of a target gene, which characteristic sequences comprises a mutation that is not found in other copies of the gene, e.g., the wild-type copy of the gene, another mutant copy the gene, etc. In some embodiments, a common base sequence comprises or is a sequence 100% complementary to characteristic sequence element of a target gene, which characteristic sequences comprises a mutation that is not found in other copies of the gene, e.g., the wild-type copy of the gene, another mutant copy the gene, etc.

In some embodiments, a common base sequence comprises or is a sequence complementary to a sequence comprising an SNP. In some embodiments, a common base sequence comprises or is a sequence complementary to a sequence comprising an SNP, and the common base sequence is 100% complementary to the SNP that is associated with a disease.

In some embodiments, a chiral internucleotidic linkage in provided oligonucleotides has the structure of formula I. In some embodiments, a chiral internucleotidic linkage is phosphorothioate. In some embodiments, each chiral internucleotidic linkage in a single oligonucleotide of a provided composition independently has the structure of formula I. In some embodiments, each chiral internucleotidic linkage in a single oligonucleotide of a provided composition is a phosphorothioate.

In some embodiments, oligonucleotides of the present disclosure comprise one or more modified sugar moieties. In some embodiments, oligonucleotides of the present disclosure comprise one or more modified base moieties. As known by a person of ordinary skill in the art and described in the disclosure, various modifications can be introduced to a sugar and/or moiety. For example, in some embodiments, a modification is a modification described in U.S. Pat. No. 9,006,198, WO2014/012081 and WO/2015/107425, the sugar and base modifications of each of which are incorporated herein by reference.

In some embodiments, a sugar modification is a 2′-modification. Commonly used 2′-modifications include but are not limited to 2′-OR¹, wherein R¹ is not hydrogen. In some embodiments, a modification is 2′-OR, wherein R is optionally substituted aliphatic. In some embodiments, a modification is 2′-OMe. In some embodiments, a modification is 2′-O— MOE. In some embodiments, the present disclosure demonstrates that inclusion and/or location of particular chirally pure internucleotidic linkages can provide stability improvements comparable to or better than those achieved through use of modified backbone linkages, bases, and/or sugars. In some embodiments, a provided single oligonucleotide of a provided composition has no modifications on the sugars. In some embodiments, a provided single oligonucleotide of a provided composition has no modifications on 2′-positions of the sugars (i.e., the two groups at the 2′-position are either —H/—H or —H/—OH). In some embodiments, a provided single oligonucleotide of a provided composition does not have any 2′-MOE modifications.

In some embodiments, a 2′-modification is —O-L- or -L- which connects the 2′-carbon of a sugar moiety to another carbon of a sugar moiety. In some embodiments, a 2′-modification is —O-L- or -L- which connects the 2′-carbon of a sugar moiety to the 4′-carbon of a sugar moiety. In some embodiments, a 2′-modification is S-cEt. In some embodiments, a modified sugar moiety is an LNA moiety.

In some embodiments, a 2′-modification is —F. In some embodiments, a 2′-modification is FANA. In some embodiments, a 2′-modification is FRNA.

In some embodiments, a sugar modification is a 5′-modification, e.g., R-5′-Me, S-5′-Me, etc.

In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Example such moieties are widely known in the art, including but not limited to those used in morpholino (optionally with its phosphorodiamidate linkage), glycol nucleic acids, etc.

In some embodiments, a provided oligonucleotide in a provided composition has at least about 25% of its internucleotidic linkages in Sp configuration. In some embodiments, a provided oligonucleotide in a provided composition has at least about 30% of its internucleotidic linkages in Sp configuration. In some embodiments, a provided oligonucleotide in a provided composition has at least about 35% of its internucleotidic linkages in Sp configuration. In some embodiments, a provided oligonucleotide in a provided composition has at least about 40% of its internucleotidic linkages in Sp configuration. In some embodiments, a provided oligonucleotide in a provided composition has at least about 45% of its internucleotidic linkages in Sp configuration. In some embodiments, a provided oligonucleotide in a provided composition has at least about 50% of its internucleotidic linkages in Sp configuration. In some embodiments, a provided oligonucleotide in a provided composition has at least about 55% of its internucleotidic linkages in Sp configuration. In some embodiments, a provided oligonucleotide in a provided composition has at least about 60% of its internucleotidic linkages in Sp configuration. In some embodiments, a provided oligonucleotide in a provided composition has at least about 65% of its internucleotidic linkages in Sp configuration. In some embodiments, a provided oligonucleotide in a provided composition has at least about 70% of its internucleotidic linkages in Sp configuration. In some embodiments, a provided oligonucleotide in a provided composition has at least about 75% of its internucleotidic linkages in Sp configuration. In some embodiments, a provided oligonucleotide in a provided composition has at least about 80% of its internucleotidic linkages in Sp configuration. In some embodiments, a provided oligonucleotide in a provided composition has at least about 85% of its internucleotidic linkages in Sp configuration. In some embodiments, a provided oligonucleotide in a provided composition has at least about 90% of its internucleotidic linkages in Sp configuration.

In some embodiments, the present disclosure provides chirally controlled oligonucleotide compositions which are of high crude purity and of high diastereomeric purity. In some embodiments, the present disclosure provides and chirally controlled oligonucleotide compositions which are of high crude purity. In some embodiments, the present disclosure provides chirally controlled oligonucleotide compositions which are of high diastereomeric purity.

In some embodiments, a chirally controlled oligonucleotide composition is a substantially pure preparation of an oligonucleotide type in that oligonucleotides in the composition that are not of the oligonucleotide type are impurities form the preparation process of said oligonucleotide type, in some case, after certain purification procedures.

In some embodiments, the present disclosure provides oligonucleotides comprising one or more diastereomerically pure internucleotidic linkages with respect to the chiral linkage phosphorus within the composition. In some embodiments, the present disclosure provides oligonucleotides comprising one or more diastereomerically pure internucleotidic linkages having the structure of formula I. In some embodiments, the present disclosure provides oligonucleotides comprising one or more diastereomerically pure internucleotidic linkages with respect to the chiral linkage phosphorus, and one or more phosphate diester linkages. In some embodiments, the present disclosure provides oligonucleotides comprising one or more diastereomerically pure internucleotidic linkages having the structure of formula I, and one or more phosphate diester linkages. In some embodiments, the present disclosure provides oligonucleotides comprising one or more diastereomerically pure internucleotidic linkages having the structure of formula I-c, and one or more phosphate diester linkages. In some embodiments, such oligonucleotides are prepared by using stereoselective oligonucleotide synthesis, as described in this application, to form pre-designed diastereomerically pure internucleotidic linkages with respect to the chiral linkage phosphorus.

In certain embodiments, a modified internucleotidic linkages has the structure of formula I:

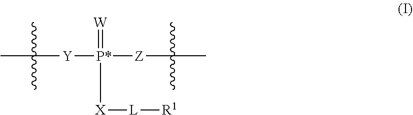

wherein each variable is as defined and described below. In some embodiments, a linkage of formula I is chiral. In some embodiments, the present disclosure provides oligonucleotides comprising one or more modified internucleotidic linkages of formula I. In some embodiments, the present disclosure provides an oligonucleotide comprising one or more modified internucleotidic linkages of formula I, and wherein individual internucleotidic linkages of formula I within the oligonucleotide have different P-modifications relative to one another. In some embodiments, the present disclosure provides an oligonucleotide comprising one or more modified internucleotidic linkages of formula I, and wherein individual internucleotidic linkages of formula I within the oligonucleotide have different —X-L-R¹ relative to one another. In some embodiments, the present disclosure provides an oligonucleotide comprising one or more modified internucleotidic linkages of formula I, and wherein individual internucleotidic linkages of formula I within the oligonucleotide have different X relative to one another. In some embodiments, the present disclosure provides an oligonucleotide comprising one or more modified internucleotidic linkages of formula I, and wherein individual internucleotidic linkages of formula I within the oligonucleotide have different -L-R¹ relative to one another.

In some embodiments, a chirally controlled oligonucleotide is an oligonucleotide in a chirally controlled composition that is of the particular oligonucleotide type, and the chirally controlled oligonucleotide is of the type. In some embodiments, a chirally controlled oligonucleotide is an oligonucleotide in a provided composition that comprises a predetermined level of a plurality of oligonucleotides that share a common base sequence, a common pattern of backbone linkages, and a common pattern of backbone chiral centers, and the chirally controlled oligonucleotide shares the common base sequence, the common pattern of backbone linkages, and the common pattern of backbone chiral centers.

In some embodiments, the present disclosure provides a chirally controlled oligonucleotide, wherein at least two of the individual internucleotidic linkages within the oligonucleotide have different stereochemistry and/or different P-modifications relative to one another. In some embodiments, the present disclosure provides a chirally controlled oligonucleotide, wherein at least two of the individual internucleotidic linkages within the oligonucleotide have different stereochemistry relative to one another, and wherein at least a portion of the structure of the chirally controlled oligonucleotide is characterized by a repeating pattern of alternating stereochemistry.

In some embodiments, the present disclosure provides a chirally controlled oligonucleotide, wherein at least two of the individual internucleotidic linkages within the oligonucleotide have different P-modifications relative to one another, in that they have different X atoms in their —XLR¹ moieties, and/or in that they have different L groups in their —XLR¹ moieties, and/or that they have different R¹ atoms in their —XLR¹ moieties.

In some embodiments, the present disclosure provides a chirally controlled oligonucleotide, wherein at least two of the individual internucleotidic linkages within the oligonucleotide have different stereochemistry and/or different P-modifications relative to one another and the oligonucleotide has a structure represented by the following formula:

[S^{B}n1R^{B}n2S^{B}n3R^{B}n4 . . . S^{B}nxR^{B}ny]

wherein:
each R^{B} independently represents a block of nucleotide units having the R configuration at the linkage phosphorus;
each S^{B} independently represents a block of nucleotide units having the S configuration at the linkage phosphorus;
each of n1-ny is zero or an integer, with the requirement that at least one odd n and at least one even n must be non-zero so that the oligonucleotide includes at least two individual internucleotidic linkages with different stereochemistry relative to one another; and
   wherein the sum of n1-ny is between 2 and 200, and in some embodiments is between a lower limit selected from the group consisting of 2, 3, 4, 5, 6, 7, 8, 9, 10, 11, 12, 13, 14, 15, 16, 17, 18, 19, 20, 21, 22, 23, 24, 25 or more and an upper limit selected from the group consisting of 5, 10, 15, 20, 25, 30, 35, 40, 45, 50, 55, 60, 65, 70, 75, 80, 85, 90, 95, 100, 110, 120, 130, 140, 150, 160, 170, 180, 190, and 200, the upper limit being larger than the lower limit.

In some such embodiments, each n has the same value; in some embodiments, each even n has the same value as each other even n; in some embodiments, each odd n has the same value each other odd n; in some embodiments, at least two even ns have different values from one another; in some embodiments, at least two odd ns have different values from one another.

In some embodiments, at least two adjacent ns are equal to one another, so that a provided oligonucleotide includes adjacent blocks of S stereochemistry linkages and R stereochemistry linkages of equal lengths. In some embodiments, provided oligonucleotides include repeating blocks of S and R stereochemistry linkages of equal lengths. In some embodiments, provided oligonucleotides include repeating blocks of S and R stereochemistry linkages, where at least two such blocks are of different lengths from one another; in some such embodiments each S stereochemistry block is of the same length, and is of a different length from each R stereochemistry length, which may optionally be of the same length as one another.

In some embodiments, at least two skip-adjacent ns are equal to one another, so that a provided oligonucleotide includes at least two blocks of linkages of a first stereochemistry that are equal in length to one another and are separated by a block of linkages of the other stereochemistry, which separating block may be of the same length or a different length from the blocks of first stereochemistry.

In some embodiments, ns associated with linkage blocks at the ends of a provided oligonucleotide are of the same length. In some embodiments, provided oligonucleotides have terminal blocks of the same linkage stereochemistry. In some such embodiments, the terminal blocks are separated from one another by a middle block of the other linkage stereochemistry.

In some embodiments, a provided oligonucleotide of formula [S^{B}n1R^{B}n2S^{B}n3R^{B}n4 . . . S^{B}nxR^{B}ny] is a stereoblockmer. In some embodiments, a provided oligonucleotide of formula [S^{B}n1R^{B}n2S^{B}n3R^{B}n4 . . . S^{B}nxR^{B}ny] is a stereoskipmer. In some embodiments, a provided oligonucleotide of formula [S^{B}n1R^{B}n2S^{B}n3R^{B}n4 . . . S^{B}nxR^{B}ny] is a stereoaltmer. In some embodiments, a provided oligonucleotide of formula [S^{B}n1R^{B}n2S^{B}n3R^{B}n4 . . . S^{B}nxR^{B}ny] is a gapmer.

In some embodiments, a provided oligonucleotide of formula [S^{B}n1R^{B}n2S^{B}n3R^{B}n4 . . . S^{B}nxR^{B}ny] is of any of the above described patterns and further comprises patterns of P-modifications. For instance, in some embodiments, a provided oligonucleotide of formula [S^{B}n1R^{B}n2S^{B}n3R^{B}n4 . . . S^{B}nxR^{B}ny] and is a stereoskipmer and P-modification skipmer. In some embodiments, a provided oligonucleotide of formula [S^{B}n1R^{B}n2S^{B}n3R^{B}n4 . . . S^{B}nxR^{B}ny] and is a stereoblockmer and P-modification altmer. In some embodiments, a provided oligonucleotide of formula [S^{B}n1R^{B}n2S^{B}n3R^{B}n4 . . . S^{B}nxR^{B}ny] and is a stereoaltmer and P-modification blockmer.

In some embodiments, a provided oligonucleotide, for example, an oligonucleotide of formula [S^{B}n1R^{B}n2S^{B}n3R^{B}n4 . . . S^{B}nxR^{B}ny], is a chirally controlled oligonucleotide comprising one or more modified internucleotidic linkages independently having the structure of formula I.

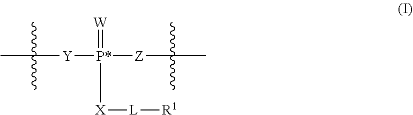

wherein:
P* is an asymmetric phosphorus atom and is either Rp or Sp;
W is O, S or Se;
each of X, Y and Z is independently —O—, —S—, —N(-L-R¹)—, or L;
L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene;
each R is independently hydrogen, or an optionally substituted group selected from C₁-C₆ aliphatic, carbocyclyl, aryl, heteroaryl, and heterocyclyl; and
each

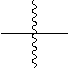

independently represents a connection to a nucleoside.

In some embodiments, a chirally controlled oligonucleotide comprises one or more modified internucleotidic phosphorus linkages. In some embodiments, a chirally controlled oligonucleotide comprises, e.g., a phosphorothioate or a phosphorothioate triester linkage. In some embodiments, a chirally controlled oligonucleotide comprises a phosphorothioate triester linkage. In some embodiments, a chirally controlled oligonucleotide comprises at least two phosphorothioate triester linkages. In some embodiments, a chirally controlled oligonucleotide comprises at least three phosphorothioate triester linkages. In some embodiments, a chirally controlled oligonucleotide comprises at least four phosphorothioate triester linkages. In some embodiments, a chirally controlled oligonucleotide comprises at least five phosphorothioate triester linkages. Example such modified internucleotidic phosphorus linkages are described further herein.

In some embodiments, a chirally controlled oligonucleotide comprises different internucleotidic phosphorus linkages. In some embodiments, a chirally controlled oligonucleotide comprises at least one phosphate diester internucleotidic linkage and at least one modified internucleotidic linkage. In some embodiments, a chirally controlled oligonucleotide comprises at least one phosphate diester internucleotidic linkage and at least one phosphorothioate triester linkage. In some embodiments, a chirally controlled oligonucleotide comprises at least one phosphate diester internucleotidic linkage and at least two phosphorothioate triester linkages. In some embodiments, a chirally controlled oligonucleotide comprises at least one phosphate diester internucleotidic linkage and at least three phosphorothioate triester linkages. In some embodiments, a chirally controlled oligonucleotide comprises at least one phosphate diester internucleotidic linkage and at least four phosphorothioate triester linkages. In some embodiments, a chirally controlled oligonucleotide comprises at least one phosphate diester internucleotidic linkage and at least five phosphorothioate triester linkages. Example such modified internucleotidic phosphorus linkages are described further herein.

In some embodiments, a phosphorothioate triester linkage comprises a chiral auxiliary, which, for example, is used to control the stereoselectivity of a reaction. In some embodiments, a phosphorothioate triester linkage does not comprise a chiral auxiliary. In some embodiments, a phosphorothioate triester linkage is intentionally maintained until and/or during the administration to a subject.

In some embodiments, a chirally controlled oligonucleotide is linked to a solid support. In some embodiments, a chirally controlled oligonucleotide is cleaved from a solid support.

In some embodiments, a chirally controlled oligonucleotide comprises at least one phosphate diester internucleotidic linkage and at least two consecutive modified internucleotidic linkages. In some embodiments, a chirally controlled oligonucleotide comprises at least one phosphate diester internucleotidic linkage and at least two consecutive phosphorothioate triester internucleotidic linkages.

In some embodiments, a chirally controlled oligonucleotide is a blockmer. In some embodiments, a chirally controlled oligonucleotide is a stereoblockmer. In some embodiments, a chirally controlled oligonucleotide is a P-modification blockmer. In some embodiments, a chirally controlled oligonucleotide is a linkage blockmer.

In some embodiments, a chirally controlled oligonucleotide is an altmer. In some embodiments, a chirally controlled oligonucleotide is a stereoaltmer. In some embodiments, a chirally controlled oligonucleotide is a P-modification altmer. In some embodiments, a chirally controlled oligonucleotide is a linkage altmer.

In some embodiments, a chirally controlled oligonucleotide is a unimer. In some embodiments, a chirally controlled oligonucleotide is a stereounimer. In some embodiments, a chirally controlled oligonucleotide is a P-modification unimer. In some embodiments, a chirally controlled oligonucleotide is a linkage unimer.

In some embodiments, a chirally controlled oligonucleotide is a gapmer.

In some embodiments, a chirally controlled oligonucleotide is a skipmer.

In some embodiments, the present disclosure provides oligonucleotides comprising one or more modified internucleotidic linkages independently having the structure of formula I:

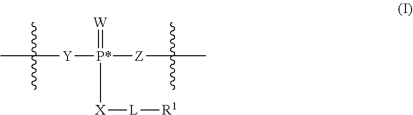

wherein:
P* is an asymmetric phosphorus atom and is either Rp or Sp;
W is O, S or Se;
each of X, Y and Z is independently —O—, —S—, —N(-L-R′)—, or L; L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene;
each R is independently hydrogen, or an optionally substituted group selected from C₁-C₆ aliphatic, carbocyclyl, aryl, heteroaryl, and heterocyclyl; and
each [symbol] independently represents a connection to a nucleoside.

In some embodiments, a modified internucleotidic linkage is phosphorothioate. Examples of internucleotidic linkages having the structure of formula (I) are widely known in the art, including but not limited to those described in US 20110294124, US 20120316224, US 20140194610, US 20150211006, US 20150197540, WO 2015107425, PCT/US2016/043542, and PCT/US2016/043598, each of which is incorporated herein by reference.

Non-limiting examples of internucleotidic linkages also include those described in the art, including, but not limited to, those described in any of: Gryaznov, S.; Chen, J.-K. J. Am. Chem. Soc. 1994, 116, 3143, Jones et al. J. Org. Chem. 1993, 58, 2983, Koshkin et al. 1998 Tetrahedron 54: 3607-3630, Lauritsen et al. 2002 Chem. Comm. 5: 530-531, Lauritsen et al. 2003 Bioo. Med. Chem. Lett. 13: 253-256, Mesmaeker et al. Angew. Chem., Int. Ed. Engl. 1994, 33, 226, Petersen et al. 2003 TRENDS Biotech. 21: 74-81, Schultz et al. 1996 Nucleic Acids Res. 24: 2966, Ts'o et al. 1988 Ann. N. Y. Acad. Sci. 507: 220, and Vasseur et al. J. Am. Chem. Soc. 1992, 114, 4006; and those described in Carbohydrate Modifications in Antisense Research; Sanghvi and Cook, eds., ACS Symposium Series 580: Chapters 3 and 4, 40-65).

In some embodiments, P* is an asymmetric phosphorus atom and is either Rp or Sp. In some embodiments, P* is Rp. In other embodiments, P* is Sp. In some embodiments, an oligonucleotide comprises one or more internucleotidic linkages of formula I wherein each P* is independently Rp or Sp. In some embodiments, an oligonucleotide comprises one or more internucleotidic linkages of formula I wherein each P* is Rp. In some embodiments, an oligonucleotide comprises one or more internucleotidic linkages of formula I wherein each P* is Sp. In some embodiments, an oligonucleotide comprises at least one internucleotidic linkage of formula I wherein P* is Rp. In some embodiments, an oligonucleotide comprises at least one internucleotidic linkage of formula I wherein P* is Sp. In some embodiments, an oligonucleotide comprises at least one internucleotidic linkage of formula I wherein P* is Rp, and at least one internucleotidic linkage of formula I wherein P* is Sp.

In some embodiments, W is O, S, or Se. In some embodiments, W is O. In some embodiments, W is S. In some embodiments, W is Se. In some embodiments, an oligonucleotide comprises at least one internucleotidic linkage of formula I wherein W is O. In some embodiments, an oligonucleotide comprises at least one internucleotidic linkage of formula I wherein W is S. In some embodiments, an oligonucleotide comprises at least one internucleotidic linkage of formula I wherein W is Se.

In some embodiments, each R is independently hydrogen, or an optionally substituted group selected from C₁-C₆ aliphatic, phenyl, carbocyclyl, aryl, heteroaryl, and heterocyclyl.

In some embodiments, R is hydrogen. In some embodiments, R is an optionally substituted group selected from C₁-C₆ aliphatic, phenyl, carbocyclyl, aryl, heteroaryl, or heterocyclyl.

In some embodiments, R is an optionally substituted C₁-C₆ aliphatic. In some embodiments, R is an optionally substituted C₁-C₆ alkyl. In some embodiments, R is optionally substituted, linear or branched hexyl. In some embodiments, R is optionally substituted, linear or branched pentyl. In some embodiments, R is optionally substituted, linear or branched butyl. In some embodiments, R is optionally substituted, linear or branched propyl. In some embodiments, R is optionally substituted ethyl. In some embodiments, R is optionally substituted methyl.

In some embodiments, R is optionally substituted phenyl. In some embodiments, R is substituted phenyl. In some embodiments, R is phenyl.

In some embodiments, R is optionally substituted carbocyclyl. In some embodiments, R is optionally substituted C₃-C₁₀ carbocyclyl. In some embodiments, R is optionally substituted monocyclic carbocyclyl. In some embodiments, R is optionally substituted cycloheptyl. In some embodiments, R is optionally substituted cyclohexyl. In some embodiments, R is optionally substituted cyclopentyl. In some embodiments, R is optionally substituted cyclobutyl. In some embodiments, R is an optionally substituted cyclopropyl. In some embodiments, R is optionally substituted bicyclic carbocyclyl.

In some embodiments, R is an optionally substituted aryl. In some embodiments, R is an optionally substituted bicyclic aryl ring.

In some embodiments, R is an optionally substituted heteroaryl. In some embodiments, R is an optionally substituted 5-6 membered monocyclic heteroaryl ring having 1-3 heteroatoms independently selected from nitrogen, sulfur, or oxygen. In some embodiments, R is a substituted 5-6 membered monocyclic heteroaryl ring having 1-3 heteroatoms independently selected from nitrogen, oxygen, or sulfur. In some embodiments, R is an unsubstituted 5-6 membered monocyclic heteroaryl ring having 1-3 heteroatoms independently selected from nitrogen, sulfur, or oxygen.

In some embodiments, R is an optionally substituted 5 membered monocyclic heteroaryl ring having 1-3 heteroatoms independently selected from nitrogen, oxygen or sulfur. In some embodiments, R is an optionally substituted 6 membered monocyclic heteroaryl ring having 1-3 heteroatoms independently selected from nitrogen, oxygen, or sulfur.

In some embodiments, R is an optionally substituted 5-membered monocyclic heteroaryl ring having 1 heteroatom selected from nitrogen, oxygen, or sulfur. In some embodiments, R is selected from pyrrolyl, furanyl, or thienyl.

In some embodiments, R is an optionally substituted 5-membered heteroaryl ring having 2 heteroatoms independently selected from nitrogen, oxygen, or sulfur. In certain embodiments, R is an optionally substituted 5-membered heteroaryl ring having 1 nitrogen atom, and an additional heteroatom selected from sulfur or oxygen. Example R groups include optionally substituted pyrazolyl, imidazolyl, thiazolyl, isothiazolyl, oxazolyl or isoxazolyl.

In some embodiments, R is a 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having 2 nitrogen atoms. In certain embodiments, R is an optionally substituted 6-membered heteroaryl ring having 1 nitrogen. Example R groups include optionally substituted pyridinyl, pyrimidinyl, pyrazinyl, pyridazinyl, triazinyl, or tetrazinyl.

In certain embodiments, R is an optionally substituted 8-10 membered bicyclic heteroaryl ring having 1-4 heteroatoms independently selected from nitrogen, oxygen, or sulfur. In some embodiments, R is an optionally substituted 5,6-fused heteroaryl ring having 1-4 heteroatoms independently selected from nitrogen, oxygen, or sulfur. In other embodiments, R is an optionally substituted 5,6-fused heteroaryl ring having 1-2 heteroatoms independently selected from nitrogen, oxygen, or sulfur. In certain embodiments, R is an optionally substituted 5,6-fused heteroaryl ring having 1 heteroatom independently selected from nitrogen, oxygen, or sulfur. In some embodiments, R is an optionally substituted indolyl. In some embodiments, R is an optionally substituted azabicyclo[3.2.1]octanyl. In certain embodiments, R is an optionally substituted 5,6-fused heteroaryl ring having 2 heteroatoms independently selected from nitrogen, oxygen, or sulfur. In some embodiments, R is an optionally substituted azaindolyl. In some embodiments, R is an optionally substituted benzimidazolyl. In some embodiments, R is an optionally substituted benzothiazolyl. In some embodiments, R is an optionally substituted benzoxazolyl. In some embodiments, R is an optionally substituted indazolyl. In certain embodiments, R is an optionally substituted 5,6-fused heteroaryl ring having 3 heteroatoms independently selected from nitrogen, oxygen, or sulfur.

In certain embodiments, R is an optionally substituted 6,6-fused heteroaryl ring having 1-4 heteroatoms independently selected from nitrogen, oxygen, or sulfur. In some embodiments, R is an optionally substituted 6,6-fused heteroaryl ring having 1-2 heteroatoms independently selected from nitrogen, oxygen, or sulfur. In other embodiments, R is an optionally substituted 6,6-fused heteroaryl ring having 1 heteroatom independently selected from nitrogen, oxygen, or sulfur. In some embodiments, R is an optionally substituted quinolinyl. In some embodiments, R is an optionally substituted isoquinolinyl. According to one aspect, R is an optionally substituted 6,6-fused heteroaryl ring having 2 heteroatoms independently selected from nitrogen, oxygen, or sulfur. In some embodiments, R is a quinazoline or a quinoxaline.

In some embodiments, R is an optionally substituted heterocyclyl. In some embodiments, R is an optionally substituted 3-7 membered saturated or partially unsaturated heterocyclic ring having 1-2 heteroatoms independently selected from nitrogen, oxygen, or sulfur. In some embodiments, R is a substituted 3-7 membered saturated or partially unsaturated heterocyclic ring having 1-2 heteroatoms independently selected from nitrogen, oxygen, or sulfur. In some embodiments, R is an unsubstituted 3-7 membered saturated or partially unsaturated heterocyclic ring having 1-2 heteroatoms independently selected from nitrogen, oxygen, or sulfur.

In some embodiments, R is an optionally substituted heterocyclyl. In some embodiments, R is an optionally substituted 6 membered saturated or partially unsaturated heterocyclic ring having 1-2 heteroatoms independently selected from nitrogen, oxygen, or sulfur. In some embodiments, R is an optionally substituted 6 membered partially unsaturated heterocyclic ring having 2 heteroatoms independently selected from nitrogen, oxygen, or sulfur. In some embodiments, R is an optionally substituted 6 membered partially unsaturated heterocyclic ring having 2 oxygen atom.

In certain embodiments, R is a 3-7 membered saturated or partially unsaturated heterocyclic ring having 1-2 heteroatoms independently selected from nitrogen, oxygen, or sulfur. In certain embodiments, R is oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothiophenyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothiophenyl, or tetrahydrothiopyranyl. In some embodiments, R is an optionally substituted 5 membered saturated or partially unsaturated heterocyclic ring having 1-2 heteroatoms independently selected from nitrogen, oxygen, or sulfur.

In certain embodiments, R is an optionally substituted 5-6 membered partially unsaturated monocyclic ring having 1-2 heteroatoms independently selected from nitrogen, oxygen, or sulfur. In certain embodiments, R is an optionally substituted tetrahydropyridinyl, dihydrothiazolyl, dihydrooxazolyl, or oxazolinyl group.

In some embodiments, R is an optionally substituted 8-10 membered bicyclic saturated or partially unsaturated heterocyclic ring having 1-4 heteroatoms independently selected from nitrogen, oxygen, or sulfur. In some embodiments, R is an optionally substituted indolinyl. In some embodiments, R is an optionally substituted isoindolinyl. In some embodiments, R is an optionally substituted 1, 2, 3, 4-tetrahydroquinoline. In some embodiments, R is an optionally substituted 1, 2, 3, 4-tetrahydroisoquinoline.

In some embodiments, each R′ is independently —R, —C(O)R, —CO₂R, or —SO₂R, or:
two R′ on the same nitrogen are taken together with their intervening atoms to form an optionally substituted heterocyclic or heteroaryl ring, or
two R′ on the same carbon are taken together with their intervening atoms to form an optionally substituted aryl, carbocyclic, heterocyclic, or heteroaryl ring.

In some embodiments, R′ is —R, —C(O)R, —CO₂R, or —SO₂R, wherein R is as defined above and described herein.

In some embodiments, R′ is —R, wherein R is as defined and described above and herein. In some embodiments, R′ is hydrogen.

In some embodiments, R′ is —C(O)R, wherein R is as defined above and described herein. In some embodiments, R′ is —CO₂R, wherein R is as defined above and described herein. In some embodiments, R′ is —SO₂R, wherein R is as defined above and described herein.

In some embodiments, two R′ on the same nitrogen are taken together with their intervening atoms to form an optionally substituted heterocyclic or heteroaryl ring. In some embodiments, two R′ on the same carbon are taken together with their intervening atoms to form an optionally substituted aryl, carbocyclic, heterocyclic, or heteroaryl ring.

In some embodiments, -Cy- is an optionally substituted bivalent ring selected from carbocyclylene, arylene, heteroarylene, or heterocyclylene.

In some embodiments, -Cy- is optionally substituted phenylene. In some embodiments, -Cy- is optionally substituted carbocyclylene. In some embodiments, -Cy- is optionally substituted arylene. In some embodiments, -Cy- is optionally substituted heteroarylene. In some embodiments, -Cy- is optionally substituted heterocyclylene.

In some embodiments, each of X, Y and Z is independently —O—, —S—, —N(-L-R¹)—, or L, wherein each of L and R¹ is independently as defined above and described below.

In some embodiments, X is —O—. In some embodiments, X is —S—. In some embodiments, X is —O— or —S—. In some embodiments, an oligonucleotide comprises at least one internucleotidic linkage of formula I wherein X is —O—. In some embodiments, an oligonucleotide comprises at least one internucleotidic linkage of formula I wherein X is —S—. In some embodiments, an oligonucleotide comprises at least one internucleotidic linkage of formula I wherein X is —O—, and at least one internucleotidic linkage of formula I wherein X is —S—. In some embodiments, an oligonucleotide comprises at least one internucleotidic linkage of formula I wherein X is —O—, and at least one internucleotidic linkage of formula I wherein X is —S—, and at least one internucleotidic linkage of formula I wherein L is an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—.

In some embodiments, X is —N(-L-R′)—. In some embodiments, X is —N(R′)—. In some embodiments, X is —N(R′)—. In some embodiments, X is —N(R)—. In some embodiments, X is —NH—.

In some embodiments, X is L. In some embodiments, X is a covalent bond. In some embodiments, X is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, X is an optionally substituted C₁-C₁₀ alkylene or C₁-C₁₀ alkenylene. In some embodiments, X is methylene.

In some embodiments, Y is —O—. In some embodiments, Y is —S—.

In some embodiments, Y is —N(-L-R′)—. In some embodiments, Y is —N(R′)—. In some embodiments, Y is —N(R′)—. In some embodiments, Y is —N(R)—. In some embodiments, Y is —NH—.

In some embodiments, Y is L. In some embodiments, Y is a covalent bond. In some embodiments, Y is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, Y is an optionally substituted C₁-C₁₀ alkylene or C₁-C₁₀ alkenylene. In some embodiments, Y is methylene.

In some embodiments, Z is —O—. In some embodiments, Z is —S—.

In some embodiments, Z is —N(-L-R′)—. In some embodiments, Z is —N(R′)—. In some embodiments, Z is —N(R′)—. In some embodiments, Z is —N(R)—. In some embodiments, Z is —NH—.

In some embodiments, Z is L. In some embodiments, Z is a covalent bond. In some embodiments, Z is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, Z is an optionally substituted C₁-C₁₀ alkylene or C₁-C₁₀ alkenylene. In some embodiments, Z is methylene.

In some embodiments, L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—.

In some embodiments, L is a covalent bond. In some embodiments, L is an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—.

In some embodiments, L has the structure of -L¹-V—, wherein:
L¹ is an optionally substituted group selected from

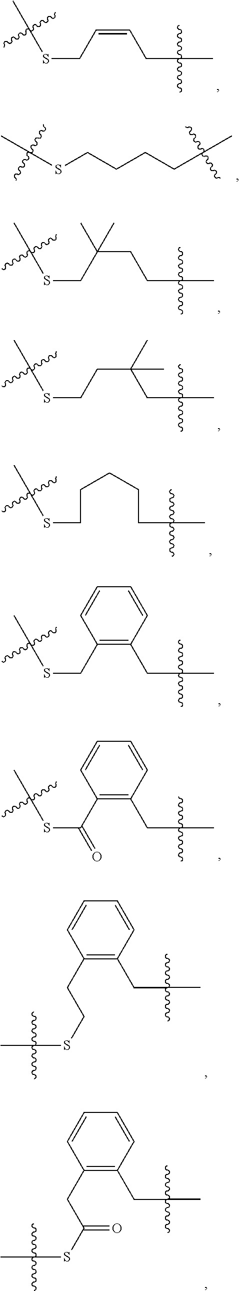

C₁-C₆ alkylene, C₁-C₆ alkenylene, carbocyclylene, arylene, C₁-C₆ heteroalkylene, heterocyclylene, and heteroarylene;
V is selected from —O—, —S—, —NR′—, C(R′)₂, —S—S—, —B—S—S—C—,

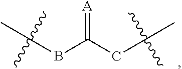

or an optionally substituted group selected from C₁-C₆ alkylene, arylene, C₁-C₆ heteroalkylene, heterocyclylene, and heteroarylene;
A is ═O, ═S, ═NR′, or ═C(R′)₂;
each of B and C is independently —O—, —S—, —NR′—, —C(R′)₂—, or an optionally substituted group selected from C₁-C₆ alkylene, carbocyclylene, arylene, heterocyclylene, or heteroarylene; and
each R′ is independently as defined above and described herein.

In some embodiments, L¹ is

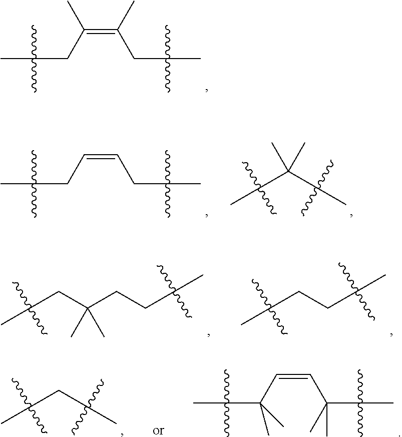

In some embodiments, L¹ is

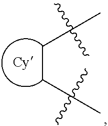

wherein Ring Cy′ is an optionally substituted arylene, carbocyclylene, heteroarylene, or heterocyclylene. In some embodiments, L¹ is optionally substituted

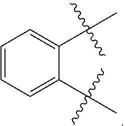 [SMILES: *c1ccccc1* ; "."]

In some embodiments, L¹ is

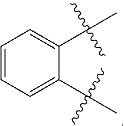 [SMILES: *c1ccccc1* ; "."]

In some embodiments, L¹ is connected to X. In some embodiments, L¹ is an optionally substituted group selected from

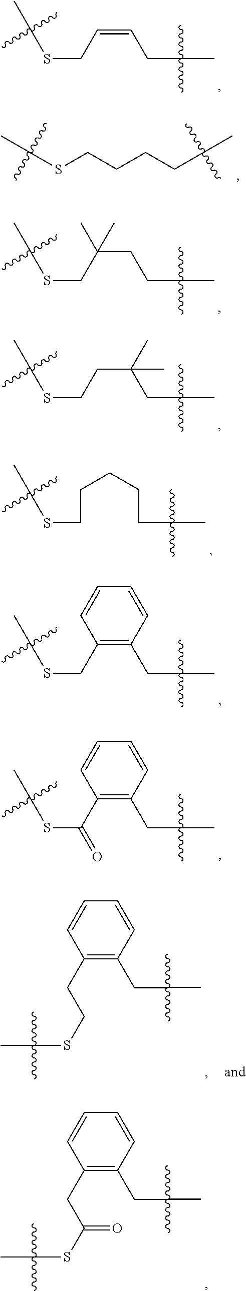

and the sulfur atom is connect to V. In some embodiments, L¹ is an optionally substituted group selected from

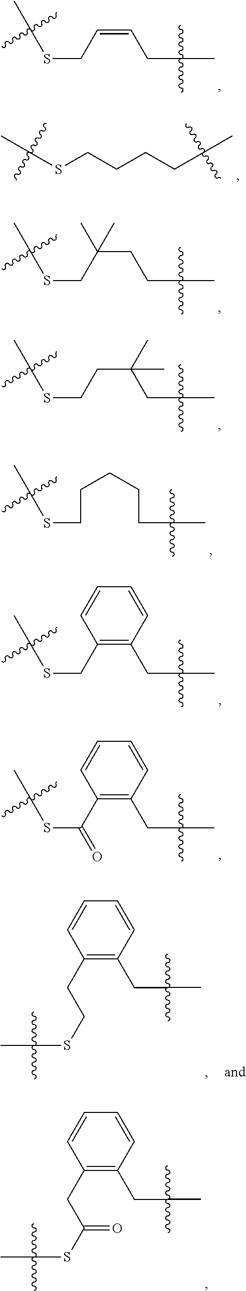

and the carbon atom is connect to X.

In some embodiments, L has the structure of:

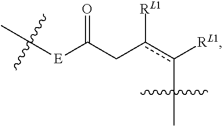

wherein:
E is —O—, —S—, —NR′— or —C(R′)₂—;
[symbol] is a single or double bond;
the two R^{L1} are taken together with the two carbon atoms to which they are bound to form an optionally substituted aryl, carbocyclic, heteroaryl or heterocyclic ring; and each R′ is independently as defined above and described herein.

In some embodiments, L has the structure of:

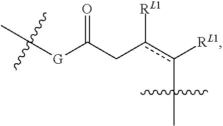

wherein:
G is —O—, —S—, or —NR′;
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, L has the structure of:

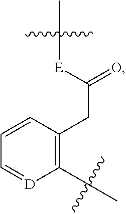

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

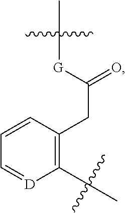

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—.

In some embodiments, L has the structure of:

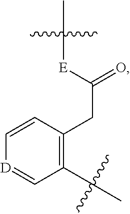

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

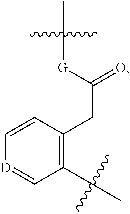

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—.

In some embodiments, L has the structure of:

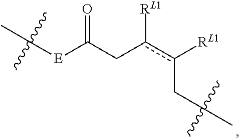

wherein:
E is —O—, —S—, —NR′— or —C(R′)₂—;
[symbol] is a single or double bond;
the two R^{L1} are taken together with the two carbon atoms to which they are bound to form an optionally substituted aryl, C₃-C₁₀ carbocyclic, heteroaryl or heterocyclic ring;
and each R′ is independently as defined above and described herein.

In some embodiments, L has the structure of:

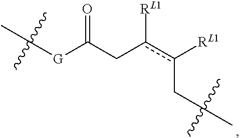

wherein:
G is —O—, —S—, or —NR′;
[symbol] is a single or double bond;
the two R^{L1} are taken together with the two carbon atoms to which they are bound to form an optionally substituted aryl, C₃-C₁₀ carbocyclic, heteroaryl or heterocyclic ring;
and each R′ is independently as defined above and described herein.

In some embodiments, L has the structure of:

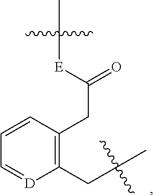

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

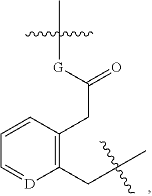

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

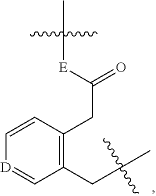

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

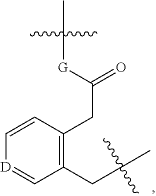

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

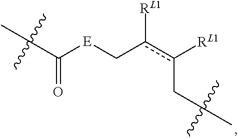

wherein:
E is —O—, —S—, —NR′— or —C(R′)₂—;
[symbol] is a single or double bond;
the two R^{L1} are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

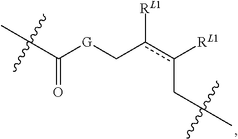

wherein:
G is —O—, —S—, or —NR′;
[symbol] is a single or double bond;
the two R^{L1} are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

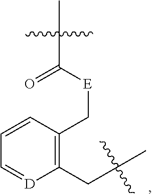

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

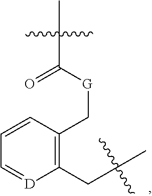

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
R′ is as defined above and described herein.

In some embodiments, L has the structure of:

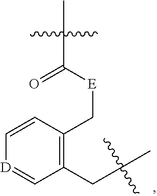

wherein:
E is —O—, —S—, —NR′— or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

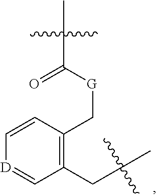

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
R′ is as defined above and described herein.

In some embodiments, L has the structure of:

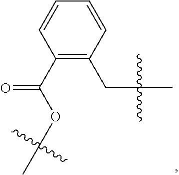 [SMILES: *Cc1ccccc1C(=O)O*]

wherein the phenyl ring is optionally substituted. In some embodiments, the phenyl ring is not substituted. In some embodiments, the phenyl ring is substituted.

In some embodiments, L has the structure of:

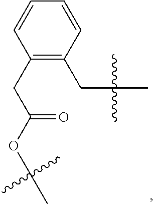 [SMILES: *Cc1ccccc1CC(=O)O*]

wherein the phenyl ring is optionally substituted. In some embodiments, the phenyl ring is not substituted. In some embodiments, the phenyl ring is substituted.

In some embodiments, L has the structure of:

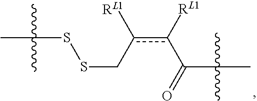

wherein:
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, L has the structure of:

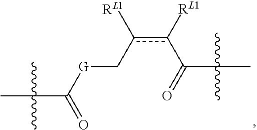

wherein:
G is —O—, —S—, or —NR′;
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, E is —O—, —S—, —NR′— or —C(R′)₂—, wherein each R′ independently as defined above and described herein. In some embodiments, E is —O—, —S—, or —NR′—. In some embodiments, E is —O—, —S—, or —NH—. In some embodiments, E is —O—. In some embodiments, E is —S—. In some embodiments, E is —NH—.

In some embodiments, G is —O—, —S—, or —NR′, wherein each R′ independently as defined above and described herein. In some embodiments, G is —O—, —S—, or —NH—. In some embodiments, G is —O—. In some embodiments, G is —S—. In some embodiments, G is —NH—.

In some embodiments, L is -L³-G-, wherein:
L³ is an optionally substituted C₁-C₅ alkylene or alkenylene, wherein one or more methylene units are optionally and independently replaced by —O—, —S—,—N(R′)—, —C(O)—, —C(S)—, —C(NR′)—, —S(O)—, —S(O)₂—, or

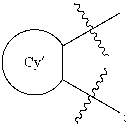 [SMILES: *CC*]

and
wherein each of G, R′ and Ring Cy′ is independently as defined above and described herein.

In some embodiments, L is -L³-S—, wherein L³ is as defined above and described herein. In some embodiments, L is -L³-O—, wherein L³ is as defined above and described herein. In some embodiments, L is -L³-N(R′)—, wherein each of L³ and R′ is independently as defined above and described herein. In some embodiments, L is -L³-NH—, wherein each of L³ and R′ is independently as defined above and described herein.

In some embodiments, L³ is an optionally substituted C₅ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

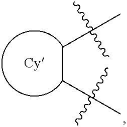 [SMILES: *CC*]

and each of R′ and Ring Cy′ is independently as defined above and described herein. In some embodiments, L³ is an optionally substituted C₅ alkylene. In some embodiments, -L³-G- is

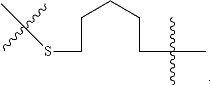 [SMILES: *CCCCCS*]

In some embodiments, L³ is an optionally substituted C₄ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

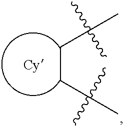 [SMILES: *CC*]

and each of R′ and Cy′ is independently as defined above and described herein.

In some embodiments, -L³-G- is

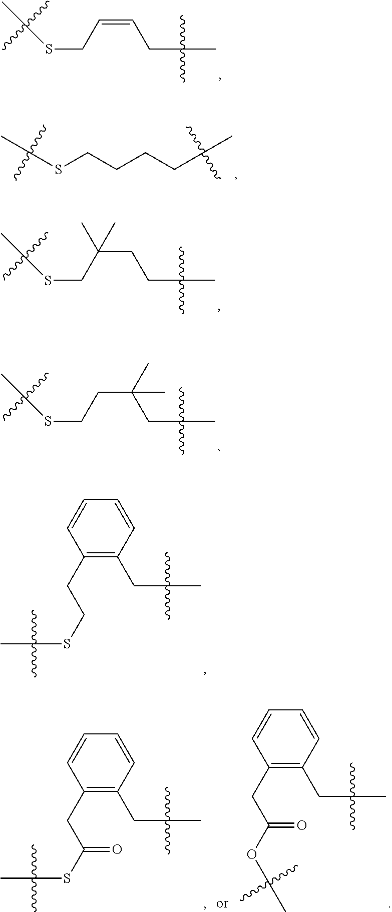

In some embodiments, L³ is an optionally substituted C₃ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

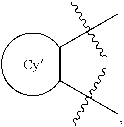 [SMILES: *CC*]

and each of R′ and Cy′ is independently as defined above and described herein.

In some embodiments, -L³-G- is

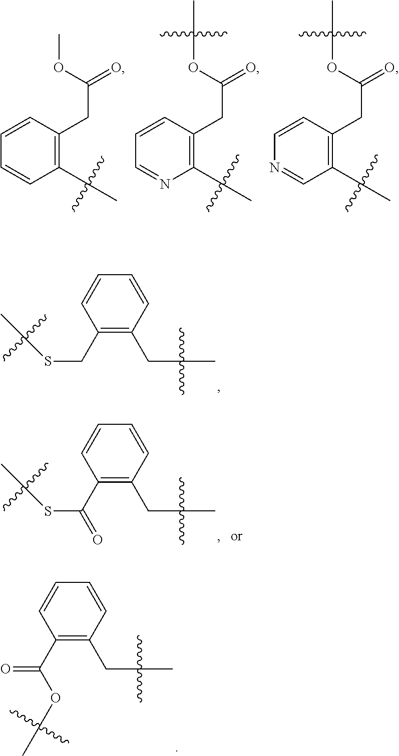

In some embodiments, L is

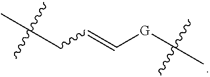

In some embodiments, L is

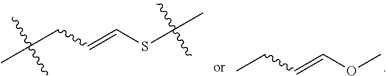

In some embodiments, L is

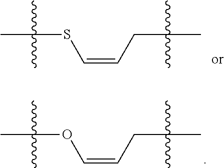 [SMILES: *C/C=C\S* ; *C/C=C\O*]

In some embodiments, L³ is an optionally substituted C₂ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

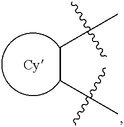 [SMILES: *CC*]

and each of R′ and Cy′ is independently as defined above and described herein.

In some embodiments, -L³-G- is

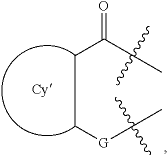

wherein each of G and Cy′ is independently as defined above and described herein. In some embodiments, L is

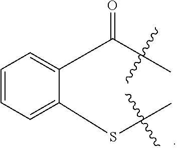 [SMILES: *Sc1ccccc1C(*)=O]

In some embodiments, L is -L⁴-G-, wherein L⁴ is an optionally substituted C₁-C₂ alkylene; and G is as defined above and described herein. In some embodiments, L is -L⁴-G-, wherein L⁴ is an optionally substituted C₁-C₂ alkylene; G is as defined above and described herein; and G is connected to R². In some embodiments, L is -L⁴-G-, wherein L⁴ is an optionally substituted methylene; G is as defined above and described herein; and G is connected to R². In some embodiments, L is -L⁴-G-, wherein L⁴ is methylene; G is as defined above and described herein; and G is connected to R². In some embodiments, L is -L⁴-G-, wherein L⁴ is an optionally substituted —(CH₂)₂—; G is as defined above and described herein; and G is connected to R². In some embodiments, L is -L⁴-G-, wherein L⁴ is —(CH₂)₂—; G is as defined above and described herein; and G is connected to R¹.

In some embodiments, L is

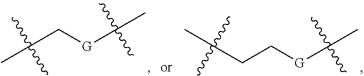

wherein G is as defined above and described herein, and G is connected to R¹. In some embodiments, L is

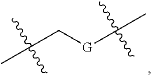

wherein G is as defined above and described herein, and G is connected to R¹. In some embodiments, L is

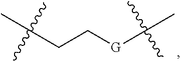

wherein G is as defined above and described herein, and G is connected to R¹. In some embodiments, L is

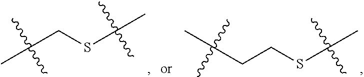

wherein the sulfur atom is connected to R¹. In some embodiments, L is

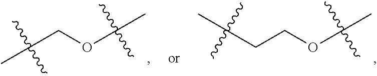

wherein the oxygen atom is connected to R¹.

In some embodiments, L is

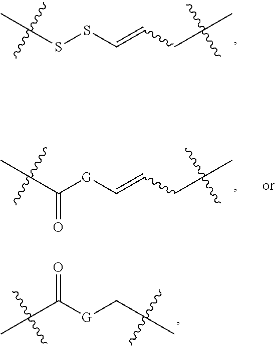

wherein G is as defined above and described herein.

In some embodiments, L is —S—R^{L3}— or —S—C(O)—R^{L3}—, wherein R^{L3} is an optionally substituted, linear or branched, C₁-C₉ alkylene, wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each of R′ and -Cy- is independently as defined above and described herein. In some embodiments, L is —S—R^{L3}— or —S—C(O)—R^{L3}—, wherein R^{L3} is an optionally substituted C₁-C₆ alkylene. In some embodiments, L is —S—R^{L3}— or —S—C(O)—R^{L3}—, wherein R^{L3} is an optionally substituted C₁-C₆ alkenylene. In some embodiments, L is —S—R^{L3}— or —S—C(O)—R^{L3}—, wherein R^{L3} is an optionally substituted C₁-C₆ alkylene wherein one or more methylene units are optionally and independently replaced by an optionally substituted C₁-C₆ alkenylene, arylene, or heteroarylene. In some embodiments, R^{L3} is an optionally substituted —S—(C₁-C₆ alkenylene)-, —S—(C₁-C₆ alkylene)-, —S—(C₁-C₆ alkylene)-arylene-(C₁-C₆ alkylene)-, —S—CO-arylene-(C₁-C₆ alkylene)-, or —S—CO—(C₁-C₆ alkylene)-arylene-(C₁-C₆ alkylene)-.

In some embodiments, L is

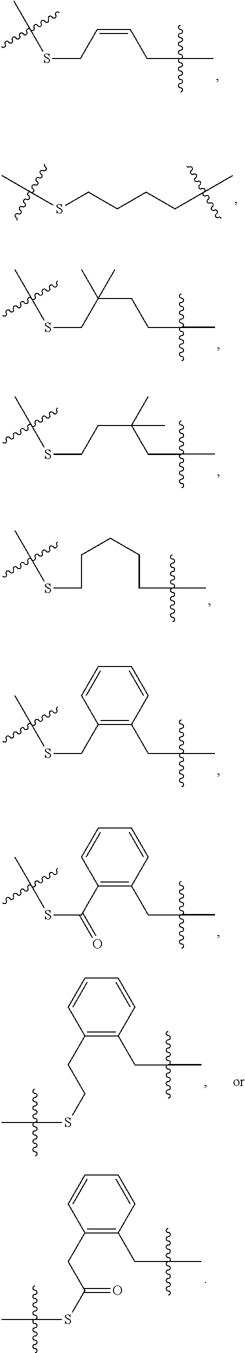

In some embodiments, L is

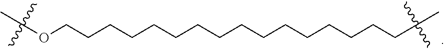 [SMILES: *CCCCCCCCCCCCCCCCO*]

In some embodiments, L is

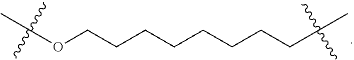 [SMILES: *CCCCCCCCO*]

In some embodiments,

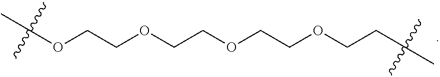 [SMILES: *CCOCCOCCOCCO*]

In some embodiments, the sulfur atom in the L embodiments described above and herein is connected to X. In some embodiments, the sulfur atom in the L embodiments described above and herein is connected to R¹.

In some embodiments, R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein.

In some embodiments, R¹ is hydrogen. In some embodiments, R¹ is halogen. In some embodiments, R¹ is —F. In some embodiments, R¹ is —Cl. In some embodiments, R¹ is —Br. In some embodiments, R¹ is —I.

In some embodiments, R¹ is R wherein R is as defined above and described herein.

In some embodiments, R¹ is hydrogen. In some embodiments, R¹ is an optionally substituted group selected from C₁-C₅₀ aliphatic, phenyl, carbocyclyl, aryl, heteroaryl, or heterocyclyl.

In some embodiments, R¹ is an optionally substituted C₁-C₅₀ aliphatic. In some embodiments, R¹ is an optionally substituted C₁-C₁₀ aliphatic. In some embodiments, R¹ is an optionally substituted C₁-C₆ aliphatic. In some embodiments, R¹ is an optionally substituted C₁-C₆ alkyl. In some embodiments, R¹ is optionally substituted, linear or branched hexyl. In some embodiments, R¹ is optionally substituted, linear or branched pentyl. In some embodiments, R¹ is optionally substituted, linear or branched butyl. In some embodiments, R¹ is optionally substituted, linear or branched propyl. In some embodiments, R¹ is optionally substituted ethyl. In some embodiments, R¹ is optionally substituted methyl.

In some embodiments, R¹ is optionally substituted phenyl. In some embodiments, R¹ is substituted phenyl. In some embodiments, R¹ is phenyl.

In some embodiments, R¹ is optionally substituted carbocyclyl. In some embodiments, R¹ is optionally substituted C₃-C₁₀ carbocyclyl. In some embodiments, R¹ is optionally substituted monocyclic carbocyclyl. In some embodiments, R¹ is optionally substituted cycloheptyl. In some embodiments, R¹ is optionally substituted cyclohexyl. In some embodiments, R¹ is optionally substituted cyclopentyl. In some embodiments, R¹ is optionally substituted cyclobutyl. In some embodiments, R¹ is an optionally substituted cyclopropyl. In some embodiments, R¹ is optionally substituted bicyclic carbocyclyl.

In some embodiments, R¹ is an optionally substituted C₁-C₅₀ polycyclic hydrocarbon. In some embodiments, R¹ is an optionally substituted C₁-C₅₀ polycyclic hydrocarbon wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is optionally substituted

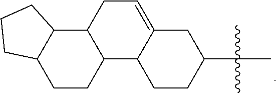 [SMILES: *C1CCC2C(=CCC3C4CCCC4CCC23)C1]

In some embodiments, R¹ is

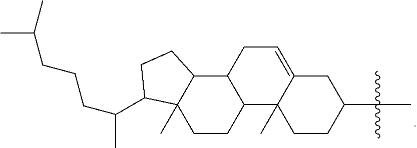 [SMILES: *C1CCC2(C)C(=CCC3C2CCC2(C)C(C(C)CCCC(C)C)CCC32)C1]

In some embodiments, R¹ is optionally substituted

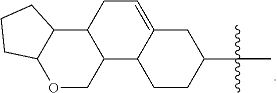 [SMILES: *C1CCC2C(=CCC3C4CCCC4OCC23)C1]

In some embodiments, R¹ is an optionally substituted C₁-C₅₀ aliphatic comprising one or more optionally substituted polycyclic hydrocarbon moieties. In some embodiments, R¹ is an optionally substituted C₁-C₅₀ aliphatic comprising one or more optionally substituted polycyclic hydrocarbon moieties, wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is an optionally substituted C₁-C₅₀ aliphatic comprising one or more optionally substituted

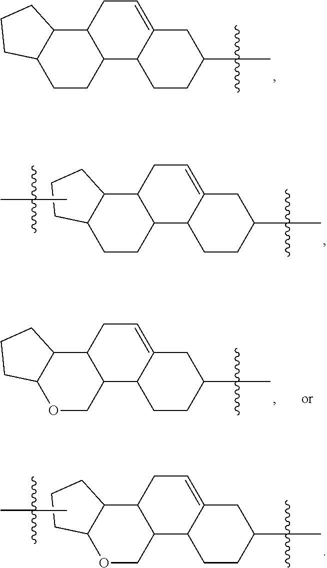

In some embodiments, R¹ is

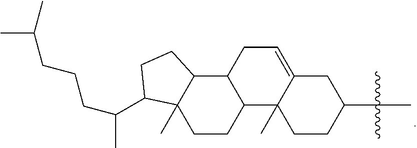 [SMILES: *C1CCC2(C)C(=CCC3C2CCC2(C)C(C(C)CCCC(C)C)CCC32)C1]

In some embodiments R¹ is

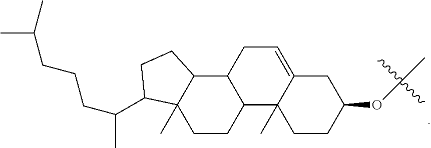 [SMILES: *O[C@H]1CCC2(C)C(=CCC3C2CCC2(C)C(C(C)CCCC(C)C)CCC32)C1]

In some embodiments, R¹ is

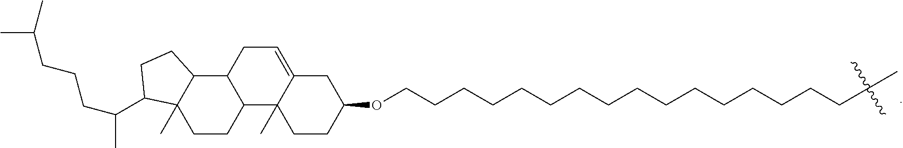 [SMILES: *CCCCCCCCCCCCCCCCO[C@H]1CCC2(C)C(=CCC3C2CCC2(C)C(C(C)CCCC(C)C)CCC32)C1]

In some embodiments, R¹ is

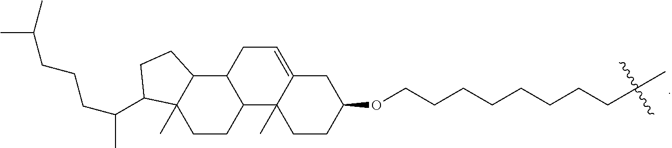 [SMILES: *CCCCCCCCO[C@H]1CCC2(C)C(=CCC3C2CCC2(C)C(C(C)CCCC(C)C)CCC32)C1]

In some embodiments, R¹ is

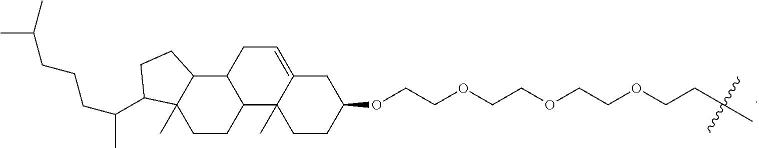 [SMILES: *CCOCCOCCOCCO[C@H]1CCC2(C)C(=CCC3C2CCC2(C)C(C(C)CCCC(C)C)CCC32)C1]

In some embodiments, R¹ is an optionally substituted aryl. In some embodiments, R¹ is an optionally substituted bicyclic aryl ring.

In some embodiments, R¹ is an optionally substituted heteroaryl. In some embodiments, R¹ is an optionally substituted 5-6 membered monocyclic heteroaryl ring having 1-3 heteroatoms independently selected from nitrogen, sulfur, or oxygen. In some embodiments, R¹ is a substituted 5-6 membered monocyclic heteroaryl ring having 1-3 heteroatoms independently selected from nitrogen, oxygen, or sulfur. In some embodiments, R¹ is an unsubstituted 5-6 membered monocyclic heteroaryl ring having 1-3 heteroatoms independently selected from nitrogen, sulfur, or oxygen.

In some embodiments, R¹ is an optionally substituted 5 membered monocyclic heteroaryl ring having 1-3 heteroatoms independently selected from nitrogen, oxygen or sulfur. In some embodiments, R¹ is an optionally substituted 6 membered monocyclic heteroaryl ring having 1-3 heteroatoms independently selected from nitrogen, oxygen, or sulfur.

In some embodiments, R¹ is an optionally substituted 5-membered monocyclic heteroaryl ring having 1 heteroatom selected from nitrogen, oxygen, or sulfur. In some embodiments, R¹ is selected from pyrrolyl, furanyl, or thienyl.

In some embodiments, R¹ is an optionally substituted 5-membered heteroaryl ring having 2 heteroatoms independently selected from nitrogen, oxygen, or sulfur. In certain embodiments, R¹ is an optionally substituted 5-membered heteroaryl ring having 1 nitrogen atom, and an additional heteroatom selected from sulfur or oxygen. Example R¹ groups include optionally substituted pyrazolyl, imidazolyl, thiazolyl, isothiazolyl, oxazolyl or isoxazolyl.

In some embodiments, R¹ is a 6-membered heteroaryl ring having 1-3 nitrogen atoms. In other embodiments, R¹ is an optionally substituted 6-membered heteroaryl ring having 1-2 nitrogen atoms. In some embodiments, R¹ is an optionally substituted 6-membered heteroaryl ring having 2 nitrogen atoms. In certain embodiments, R¹ is an optionally substituted 6-membered heteroaryl ring having 1 nitrogen. Example R¹ groups include optionally substituted pyridinyl, pyrimidinyl, pyrazinyl, pyridazinyl, triazinyl, or tetrazinyl.

In certain embodiments, R¹ is an optionally substituted 8-10 membered bicyclic heteroaryl ring having 1-4 heteroatoms independently selected from nitrogen, oxygen, or sulfur. In some embodiments, R¹ is an optionally substituted 5,6-fused heteroaryl ring having 1-4 heteroatoms independently selected from nitrogen, oxygen, or sulfur. In other embodiments, R¹ is an optionally substituted 5,6-fused heteroaryl ring having 1-2 heteroatoms independently selected from nitrogen, oxygen, or sulfur. In certain embodiments, R¹ is an optionally substituted 5,6-fused heteroaryl ring having 1 heteroatom independently selected from nitrogen, oxygen, or sulfur. In some embodiments, R¹ is an optionally substituted indolyl. In some embodiments, R¹ is an optionally substituted azabicyclo[3.2.1]octanyl. In certain embodiments, R¹ is an optionally substituted 5,6-fused heteroaryl ring having 2 heteroatoms independently selected from nitrogen, oxygen, or sulfur. In some embodiments, R¹ is an optionally substituted azaindolyl. In some embodiments, R¹ is an optionally substituted benzimidazolyl. In some embodiments, R¹ is an optionally substituted benzothiazolyl. In some embodiments, R¹ is an optionally substituted benzoxazolyl. In some embodiments, R¹ is an optionally substituted indazolyl. In certain embodiments, R¹ is an optionally substituted 5,6-fused heteroaryl ring having 3 heteroatoms independently selected from nitrogen, oxygen, or sulfur.

In certain embodiments, R¹ is an optionally substituted 6,6-fused heteroaryl ring having 1-4 heteroatoms independently selected from nitrogen, oxygen, or sulfur. In some embodiments, R¹ is an optionally substituted 6,6-fused heteroaryl ring having 1-2 heteroatoms independently selected from nitrogen, oxygen, or sulfur. In other embodiments, R¹ is an optionally substituted 6,6-fused heteroaryl ring having 1 heteroatom independently selected from nitrogen, oxygen, or sulfur. In some embodiments, R¹ is an optionally substituted quinolinyl. In some embodiments, R¹ is an optionally substituted isoquinolinyl. According to one aspect, R¹ is an optionally substituted 6,6-fused heteroaryl ring having 2 heteroatoms independently selected from nitrogen, oxygen, or sulfur. In some embodiments, R¹ is a quinazoline or a quinoxaline.

In some embodiments, R¹ is an optionally substituted heterocyclyl. In some embodiments, R¹ is an optionally substituted 3-7 membered saturated or partially unsaturated heterocyclic ring having 1-2 heteroatoms independently selected from nitrogen, oxygen, or sulfur. In some embodiments, R¹ is a substituted 3-7 membered saturated or partially unsaturated heterocyclic ring having 1-2 heteroatoms independently selected from nitrogen, oxygen, or sulfur. In some embodiments, R¹ is an unsubstituted 3-7 membered saturated or partially unsaturated heterocyclic ring having 1-2 heteroatoms independently selected from nitrogen, oxygen, or sulfur.

In some embodiments, R¹ is an optionally substituted heterocyclyl. In some embodiments, R¹ is an optionally substituted 6 membered saturated or partially unsaturated heterocyclic ring having 1-2 heteroatoms independently selected from nitrogen, oxygen, or sulfur. In some embodiments, R¹ is an optionally substituted 6 membered partially unsaturated heterocyclic ring having 2 heteroatoms independently selected from nitrogen, oxygen, or sulfur. In some embodiments, R¹ is an optionally substituted 6 membered partially unsaturated heterocyclic ring having 2 oxygen atoms.

In certain embodiments, R¹ is a 3-7 membered saturated or partially unsaturated heterocyclic ring having 1-2 heteroatoms independently selected from nitrogen, oxygen, or sulfur. In certain embodiments, R¹ is oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothiophenyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothiophenyl, or tetrahydrothiopyranyl. In some embodiments, R¹ is an optionally substituted 5 membered saturated or partially unsaturated heterocyclic ring having 1-2 heteroatoms independently selected from nitrogen, oxygen, or sulfur.

In certain embodiments, R¹ is an optionally substituted 5-6 membered partially unsaturated monocyclic ring having 1-2 heteroatoms independently selected from nitrogen, oxygen, or sulfur. In certain embodiments, R¹ is an optionally substituted tetrahydropyridinyl, dihydrothiazolyl, dihydrooxazolyl, or oxazolinyl group.

In some embodiments, R¹ is an optionally substituted 8-10 membered bicyclic saturated or partially unsaturated heterocyclic ring having 1-4 heteroatoms independently selected from nitrogen, oxygen, or sulfur. In some embodiments, R¹ is an optionally substituted indolinyl. In some embodiments, R¹ is an optionally substituted isoindolinyl. In some embodiments, R¹ is an optionally substituted 1, 2, 3, 4-tetrahydroquinoline. In some embodiments, R¹ is an optionally substituted 1, 2, 3, 4-tetrahydroisoquinoline.

In some embodiments, R¹ is an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is an optionally substituted C₁-C₁₀ aliphatic wherein one or more methylene units are optionally and independently replaced by an optionally -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—, wherein each R′ is independently as defined above and described herein. In some embodiments, R¹ is an optionally substituted C₁-C₁₀ aliphatic wherein one or more methylene units are optionally and independently replaced by an optionally -Cy-, —O—, —S—, —S—S—, —N(R′)—, —C(O)—, —OC(O)—, or —C(O)O—, wherein each R′ is independently as defined above and described herein.

In some embodiments, R¹ is

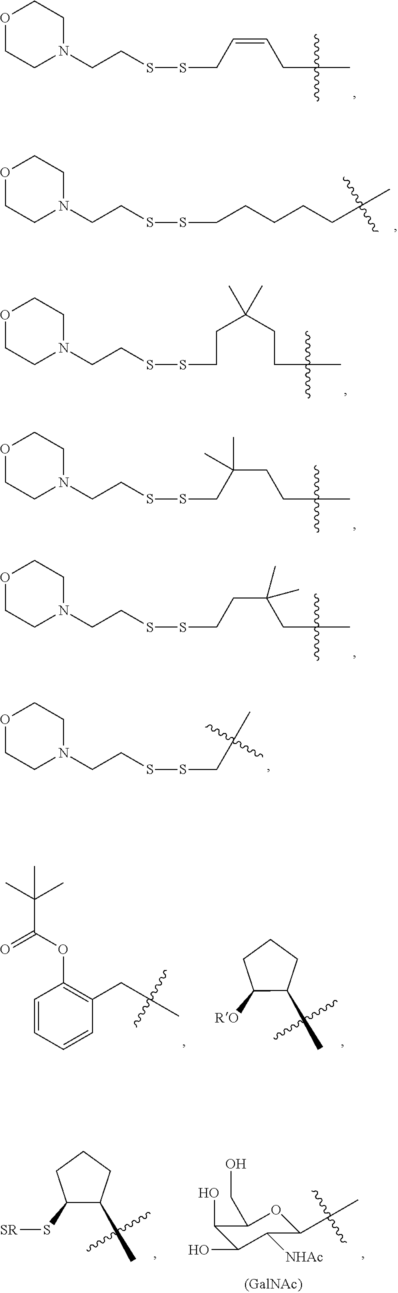

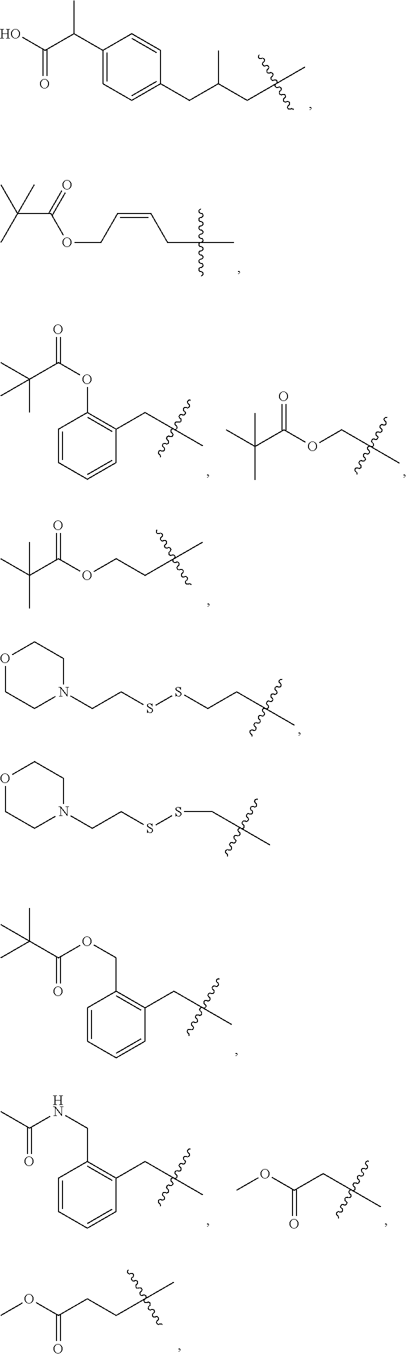

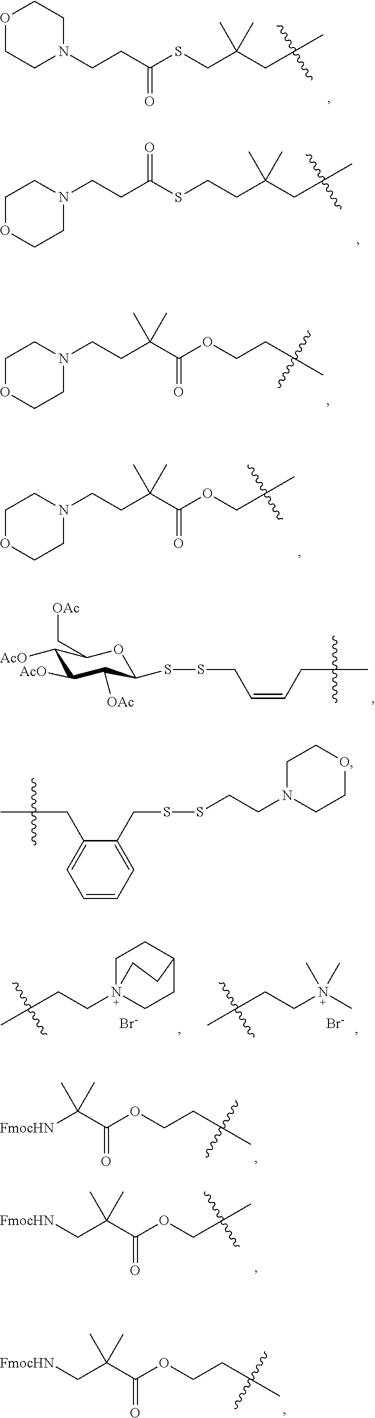

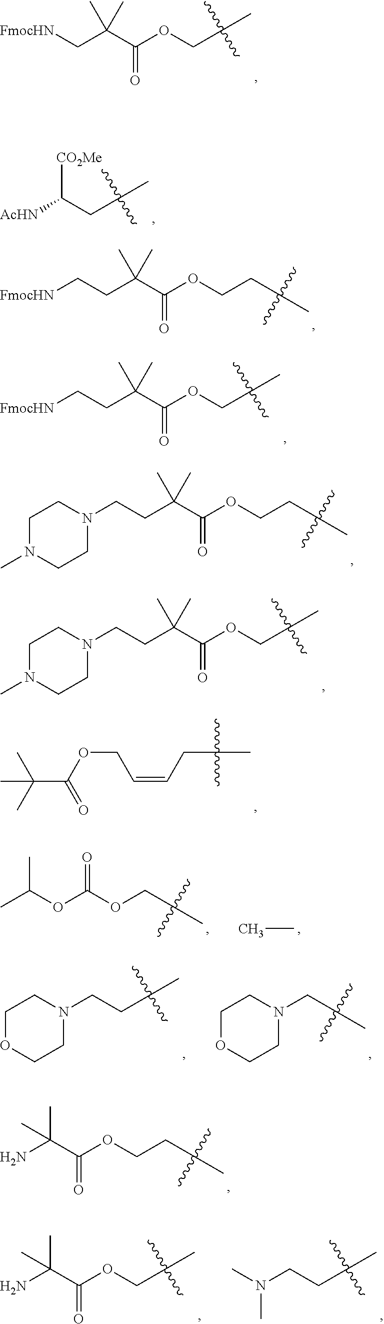

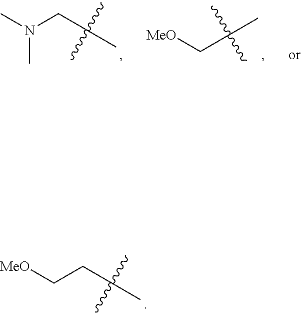

In some embodiments, R¹ is

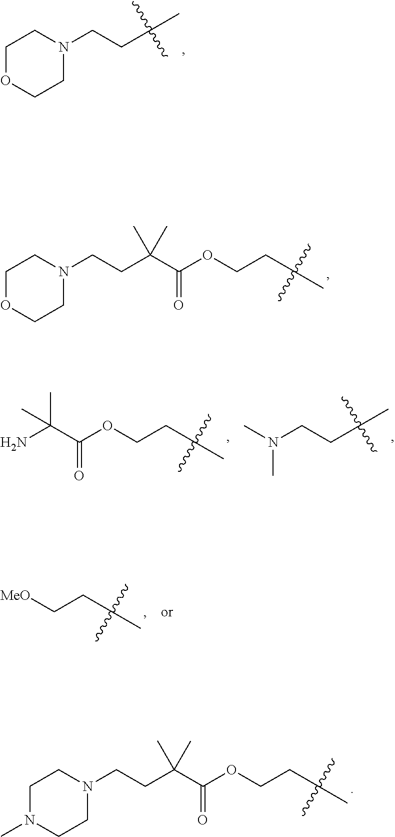

In some embodiments, R¹ comprises a terminal optionally substituted —(CH₂)₂— moiety which is connected to L. Example such R¹ groups are depicted below:

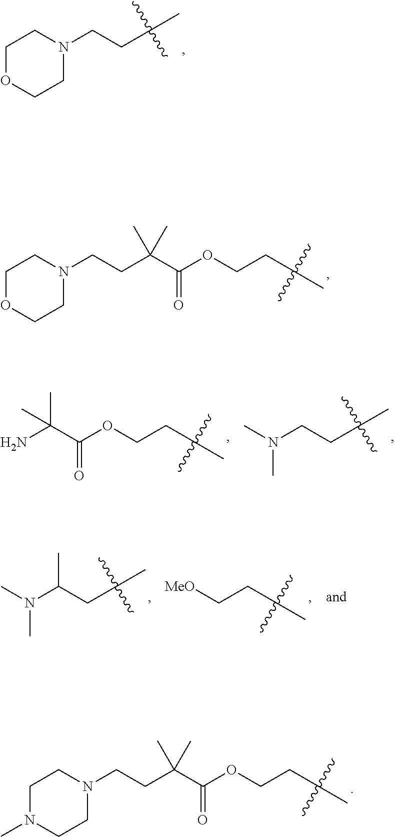

In some embodiments, R¹ comprises a terminal optionally substituted —(CH₂)— moiety which is connected to L. Exemplary such R¹ groups are depicted below:

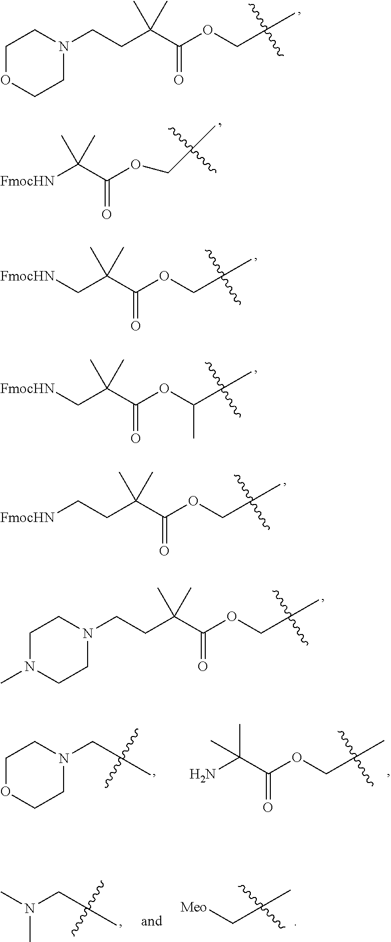

In some embodiments, R¹ is —S—R^{L2}, wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each of R′ and -Cy- is independently as defined above and described herein. In some embodiments, R¹ is —S—R^{L2}, wherein the sulfur atom is connected with the sulfur atom in L group.

In some embodiments, R¹ is —C(O)—R^{L2}, wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each of R′ and -Cy- is independently as defined above and described herein. In some embodiments, R¹ is —C(O)—R^{L2}, wherein the carbonyl group is connected with G in L group. In some embodiments, R¹ is —C(O)—R^{L2}, wherein the carbonyl group is connected with the sulfur atom in L group.

In some embodiments, R^{L2} is optionally substituted C₁-C₉ aliphatic. In some embodiments, R^{L2} is optionally substituted C₁-C₉ alkyl. In some embodiments, R^{L2} is optionally substituted C₁-C₉ alkenyl. In some embodiments, R^{L2} is optionally substituted C₁-C₉ alkynyl. In some embodiments, R^{L2} is an optionally substituted C₁-C₉ aliphatic wherein one or more methylene units are optionally and independently replaced by -Cy- or —C(O)—. In some embodiments, R^{L2} is an optionally substituted C₁-C₉ aliphatic wherein one or more methylene units are optionally and independently replaced by -Cy-. In some embodiments, R^{L2} is an optionally substituted C₁-C₉ aliphatic wherein one or more methylene units are optionally and independently replaced by an optionally substituted heterocyclylene. In some embodiments, R^{L2} is an optionally substituted C₁-C₉ aliphatic wherein one or more methylene units are optionally and independently replaced by an optionally substituted arylene. In some embodiments, R^{L2} is an optionally substituted C₁-C₉ aliphatic wherein one or more methylene units are optionally and independently replaced by an optionally substituted heteroarylene. In some embodiments, R^{L2} is an optionally substituted C₁-C₉ aliphatic wherein one or more methylene units are optionally and independently replaced by an optionally substituted C₃-C₁₀ carbocyclylene. In some embodiments, R^{L2} is an optionally substituted C₁-C₉ aliphatic wherein two methylene units are optionally and independently replaced by -Cy- or —C(O)—. In some embodiments, R^{L2} is an optionally substituted C₁-C₉ aliphatic wherein two methylene units are optionally and independently replaced by -Cy- or —C(O)—. Example R^{L2} groups are depicted below:

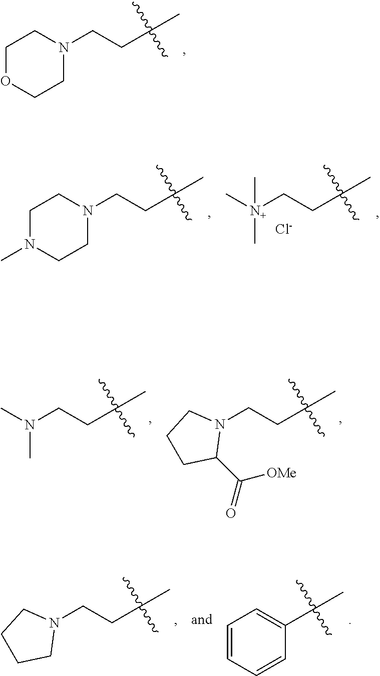

In some embodiments, R¹ is hydrogen, or an optionally substituted group selected from

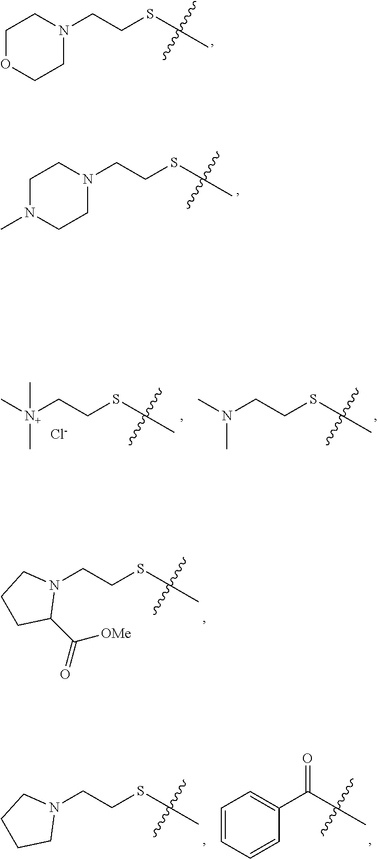

—S—(C₁-C₁₀ aliphatic), C₁-C₁₀ aliphatic, aryl, C₁-C₆ heteroalkyl, heteroaryl and heterocyclyl. In some embodiments, R¹ is

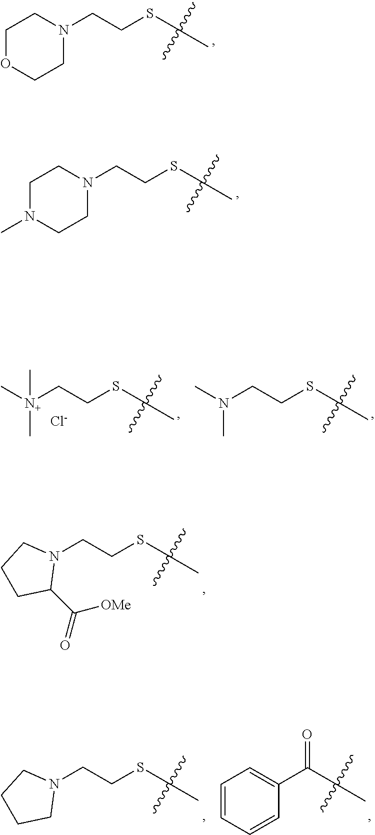

or —S—(C₁-C₁₀ aliphatic). In some embodiments, R¹ is

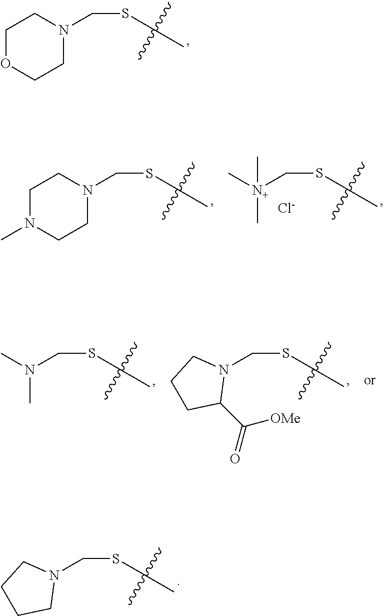

In some embodiments, R¹ is an optionally substituted group selected from —S—(C₁-C₆ aliphatic), C₁-C₁₀ aliphatic, C₁-C₆ heteroaliphatic, aryl, heterocyclyl and heteroaryl.

In some embodiments, R¹ is

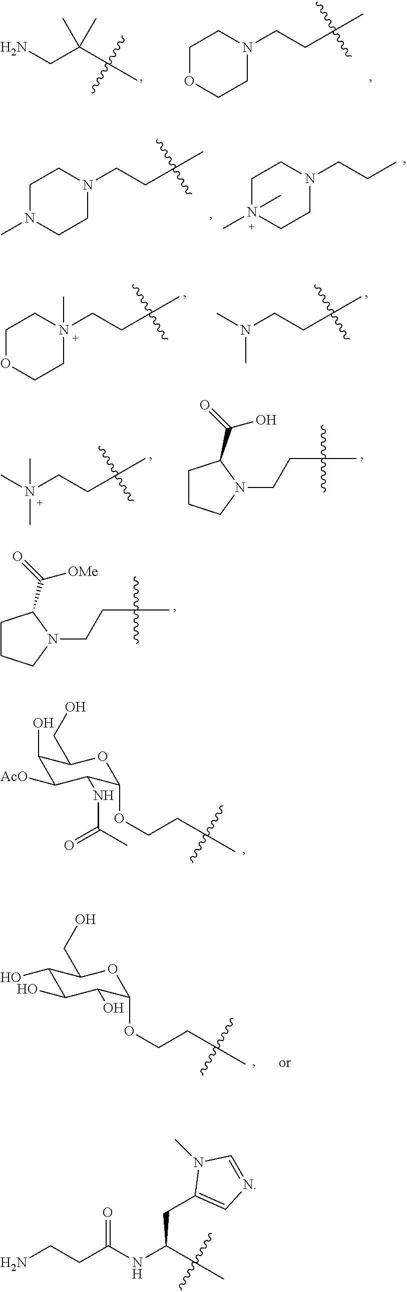

In some embodiments, the sulfur atom in the R¹ embodiments described above and herein is connected with the sulfur atom, G, E, or —C(O)— moiety in the L embodiments described above and herein. In some embodiments, the —C(O)— moiety in the R¹ embodiments described above and herein is connected with the sulfur atom, G, E, or —C(O)— moiety in the L embodiments described above and herein.

In some embodiments, -L-R¹ is any combination of the L embodiments and R¹ embodiments described above and herein.

In some embodiments, -L-R¹ is -L³-G-R¹ wherein each variable is independently as defined above and described herein.

In some embodiments, -L-R¹ is -L⁴-G-R¹ wherein each variable is independently as defined above and described herein.

In some embodiments, -L-R¹ is -L³-G-S—R^{L2}, wherein each variable is independently as defined above and described herein.

In some embodiments, -L-R¹ is -L³-G-C(O)—R^{L2}, wherein each variable is independently as defined above and described herein.

In some embodiments, -L-R¹ is

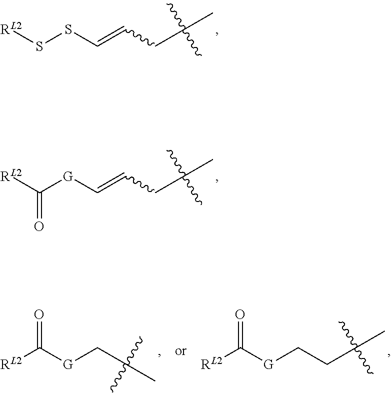

wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each G is independently as defined above and described herein.

In some embodiments, -L-R¹ is —R^{L3}—S—S—R^{L2}, wherein each variable is independently as defined above and described herein. In some embodiments, -L-R¹ is —R^{L3}—C(O)—S—S—R^{L2}, wherein each variable is independently as defined above and described herein.

In some embodiments, -L-R¹ has the structure of:

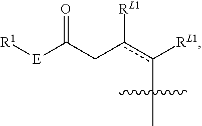

wherein each variable is independently as defined above and described herein.

In some embodiments, -L-R¹ has the structure of:

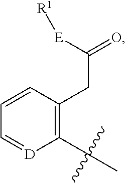

wherein each variable is independently as defined above and described herein.

In some embodiments, -L-R¹ has the structure of:

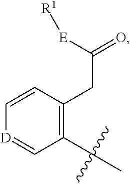

wherein each variable is independently as defined above and described herein.

In some embodiments, -L-R¹ has the structure of:

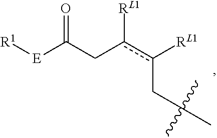

wherein each variable is independently as defined above and described herein.

In some embodiments, -L-R¹ has the structure of:

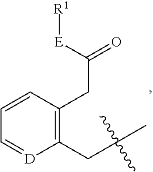

wherein each variable is independently as defined above and described herein.

In some embodiments, -L-R¹ has the structure of:

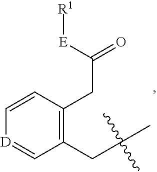

wherein each variable is independently as defined above and described herein.

In some embodiments, -L-R¹ has the structure of:

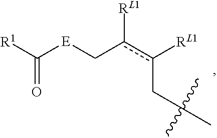

wherein each variable is independently as defined above and described herein.

In some embodiments, -L-R¹ has the structure of:

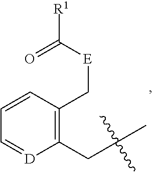

wherein each variable is independently as defined above and described herein.

In some embodiments, -L-R¹ has the structure of:

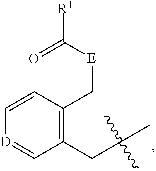

wherein each variable is independently as defined above and described herein.

In some embodiments, -L-R¹ has the structure of:

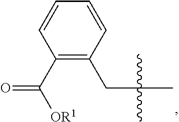

wherein each variable is independently as defined above and described herein.

In some embodiments, -L-R¹ has the structure of:

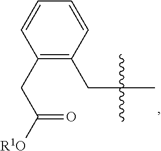

wherein each variable is independently as defined above and described herein.

In some embodiments, -L-R¹ has the structure of:

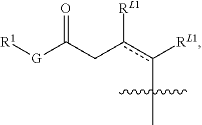

wherein each variable is independently as defined above and described herein.

In some embodiments, -L-R¹ has the structure of:

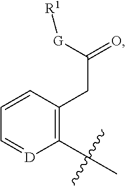

wherein each variable is independently as defined above and described herein.

In some embodiments, -L-R¹ has the structure of:

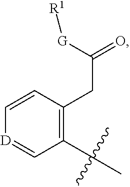

wherein each variable is independently as defined above and described herein.

In some embodiments, -L-R¹ has the structure of:

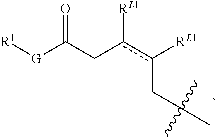

wherein each variable is independently as defined above and described herein.

In some embodiments, -L-R¹ has the structure of:

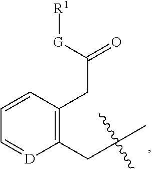

wherein each variable is independently as defined above and described herein.

In some embodiments, -L-R¹ has the structure of:

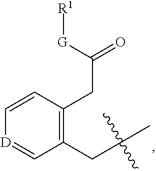

wherein each variable is independently as defined above and described herein.

In some embodiments, -L-R¹ has the structure of:

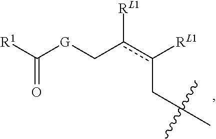

wherein each variable is independently as defined above and described herein.

In some embodiments, -L-R¹ has the structure of:

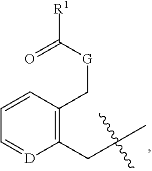

wherein each variable is independently as defined above and described herein.

In some embodiments, -L-R¹ has the structure of:

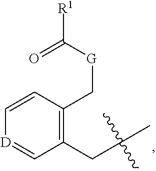

wherein each variable is independently as defined above and described herein.

In some embodiments, -L-R¹ has the structure of:

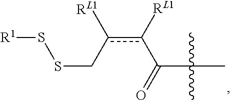

wherein each variable is independently as defined above and described herein.

In some embodiments, L has the structure of:

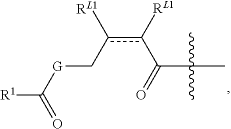

wherein each variable is independently as defined above and described herein.

In some embodiments, —X-L-R¹ has the structure of:

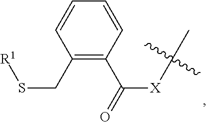

wherein:

the phenyl ring is optionally substituted, and

each of R¹ and X is independently as defined above and described herein.

In some embodiments, -L-R¹ is

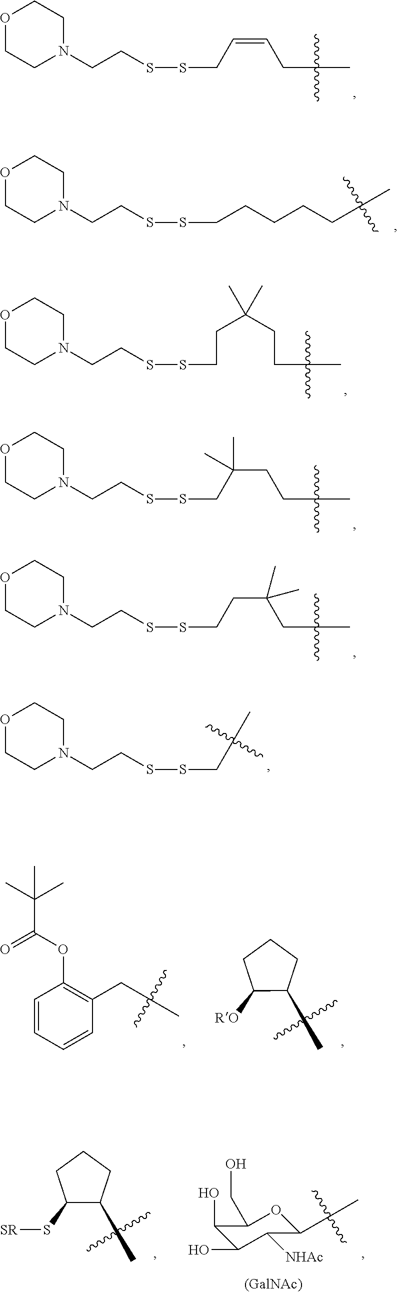

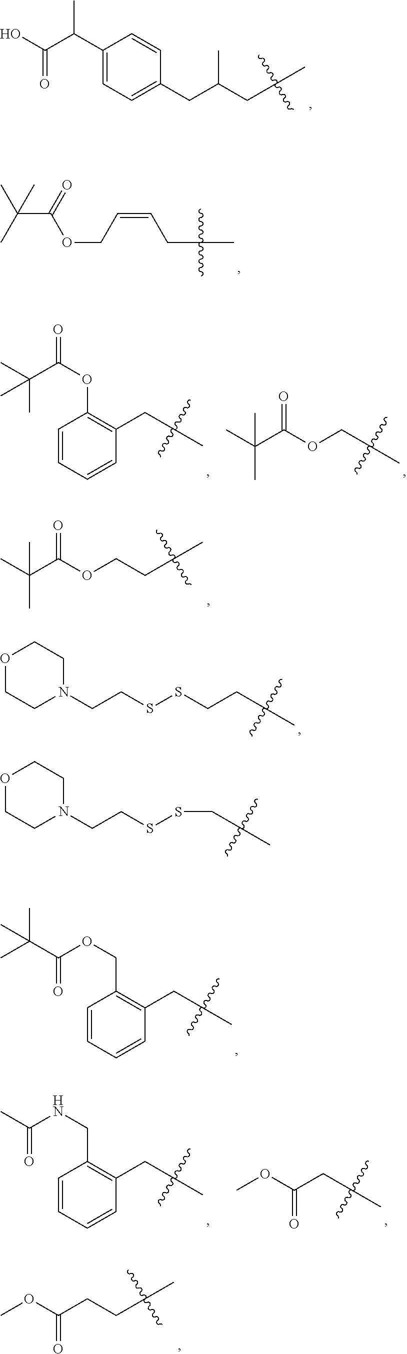

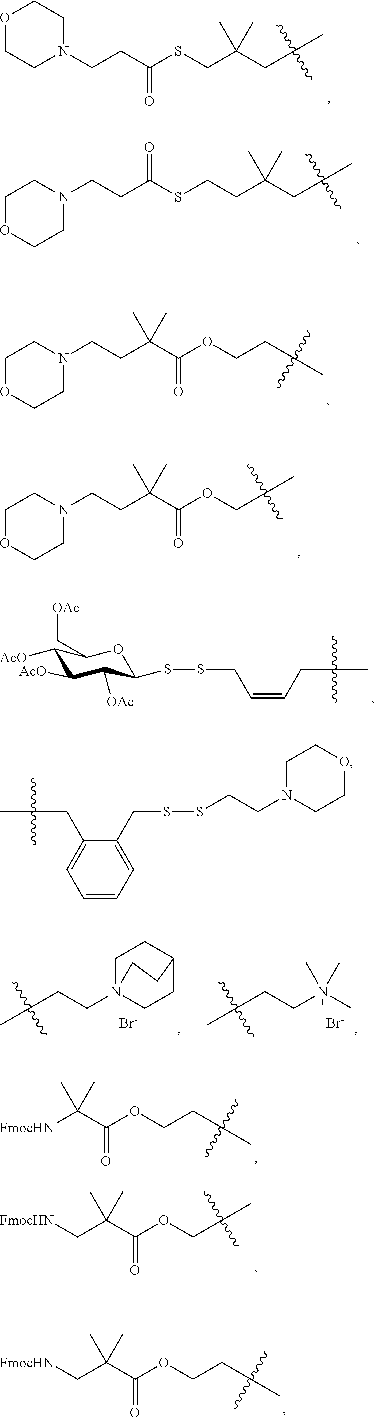

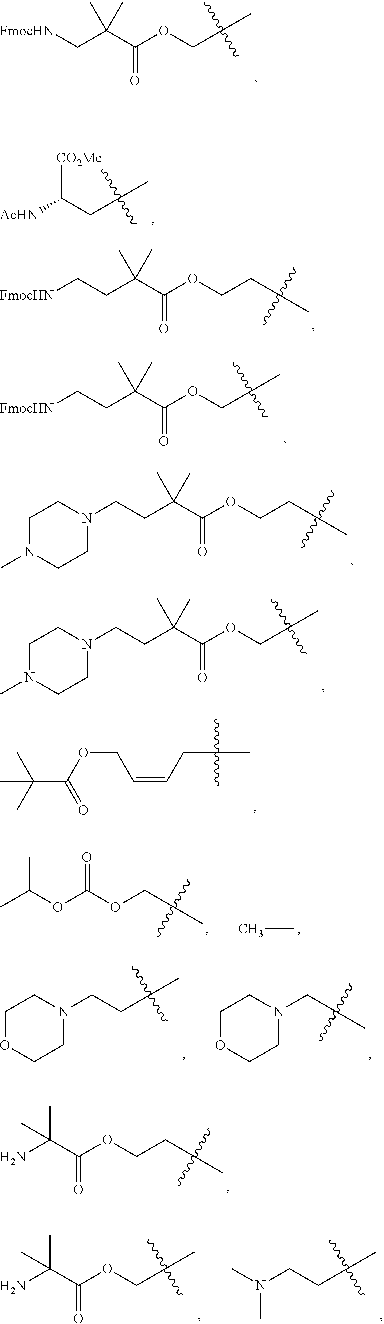

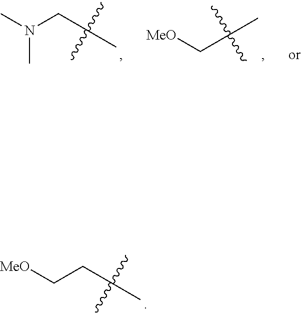

In some embodiments, -L-R¹ is:

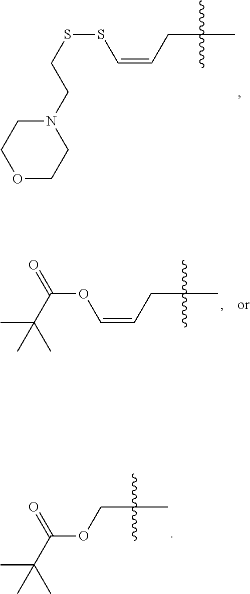

In some embodiments, -L-R¹ is CH₃—,

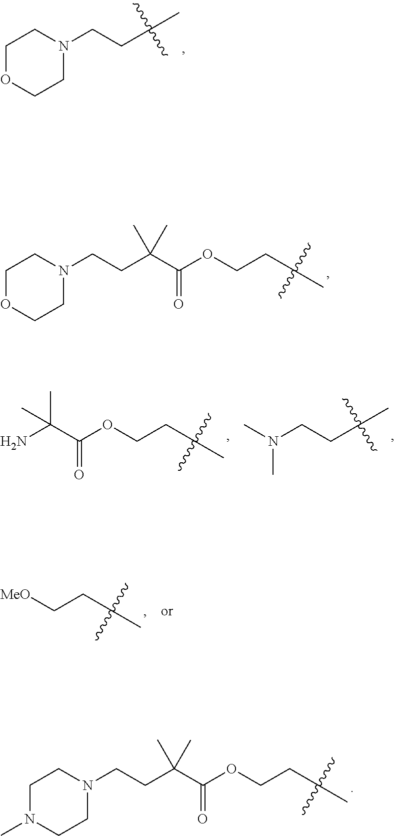

In some embodiments, -L-R¹ is

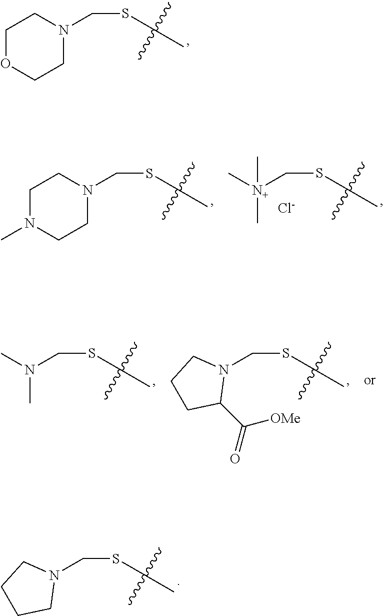

In some embodiments, -L-R¹ comprises a terminal optionally substituted —(CH₂)₂— moiety which is connected to X. In some embodiments, -L-R¹ comprises a terminal—(CH₂)₂— moiety which is connected to X. Example such -L-R¹ moieties are depicted below:

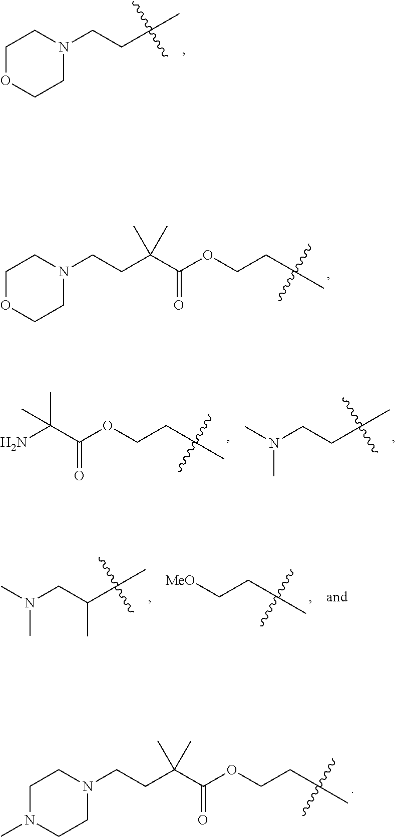

In some embodiments, -L-R¹ comprises a terminal optionally substituted —(CH₂)— moiety which is connected to X. In some embodiments, -L-R¹ comprises a terminal—(CH₂)— moiety which is connected to X. Example such -L-R¹ moieties are depicted below:

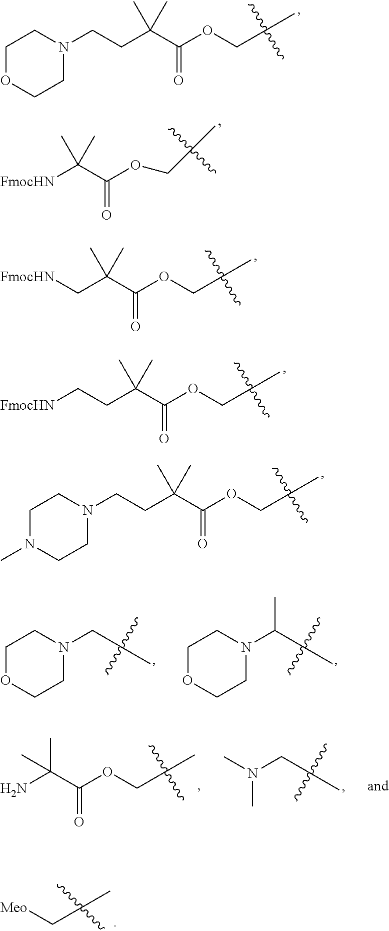

In some embodiments, -L-R is

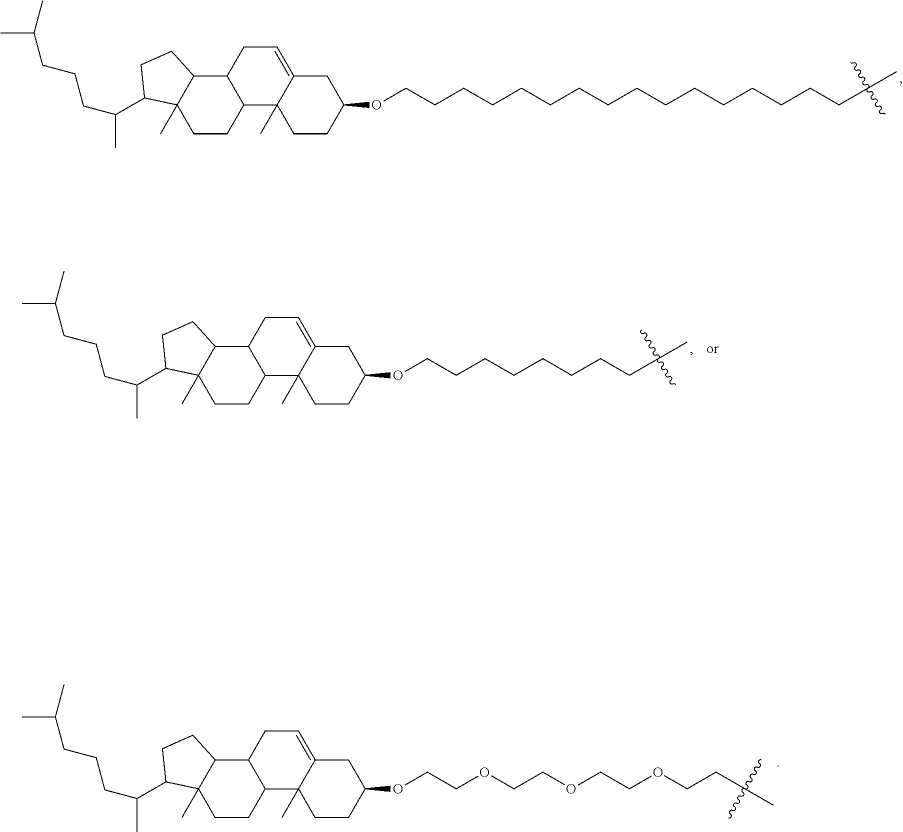 [SMILES: *CCCCCCCCCCCCCCCCO[C@H]1CCC2(C)C(=CCC3C2CCC2(C)C(C(C)CCCC(C)C)CCC32)C1 ; *CCCCCCCCO[C@H]1CCC2(C)C(=CCC3C2CCC2(C)C(C(C)CCCC(C)C)CCC32)C1 ; *CCOCCOCCOCCO[C@H]1CCC2(C)C(=CCC3C2CCC2(C)C(C(C)CCCC(C)C)CCC32)C1]

In some embodiments, -L-R¹ is CH₃—,

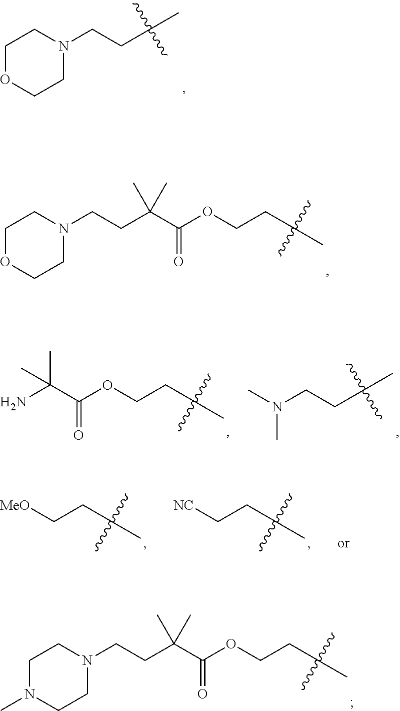

### and X is —S—.

In some embodiments, -L-R¹ is CH₃—,

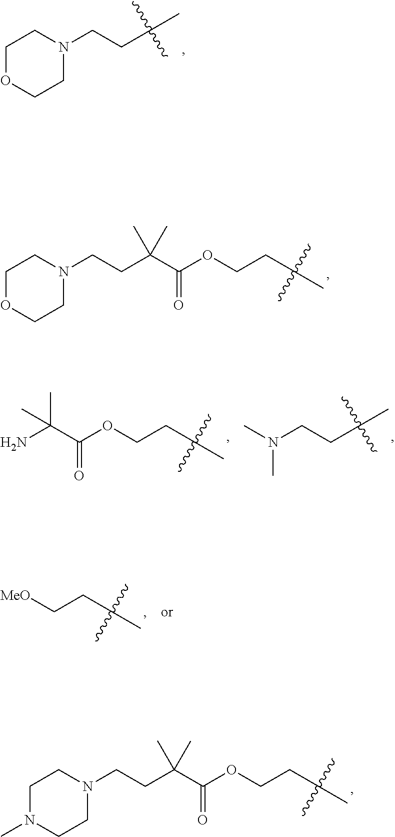

### X is —S—, W is O, Y is —O—, and Z is —O—.

In some embodiments, R¹ is

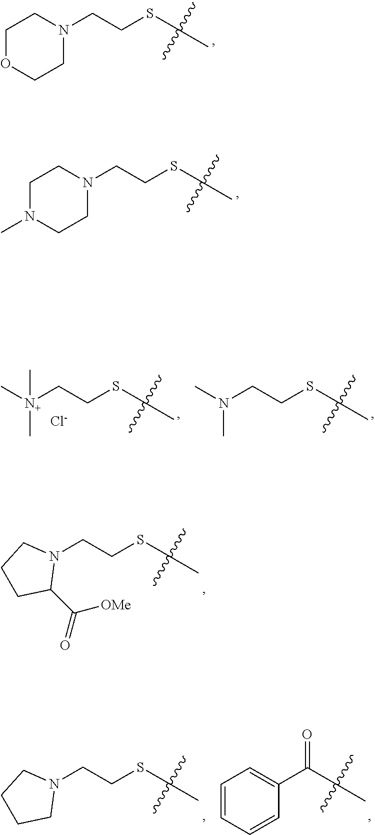

or —S—(C₁-C₁₀ aliphatic).

In some embodiments, R¹ is

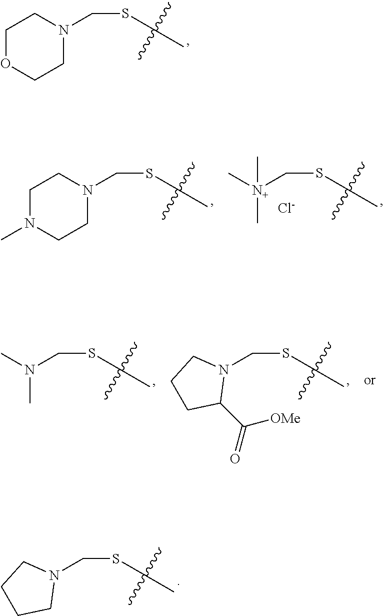

In some embodiments, X is —O— or —S—, and R¹ is

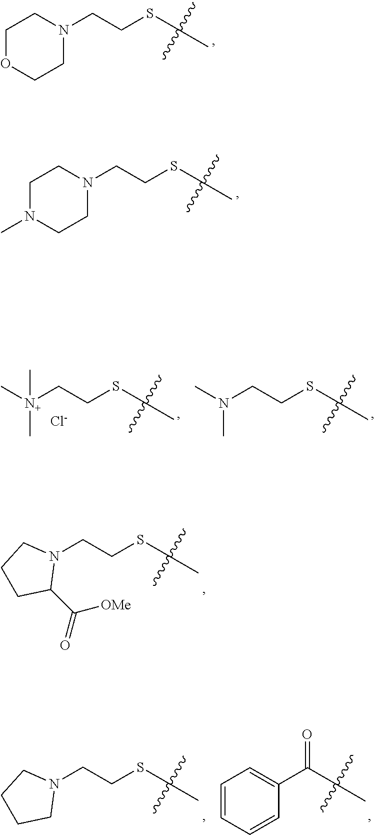

or —S—(C₁-C₁₀ aliphatic).

In some embodiments, X is —O— or —S—, and R¹ is

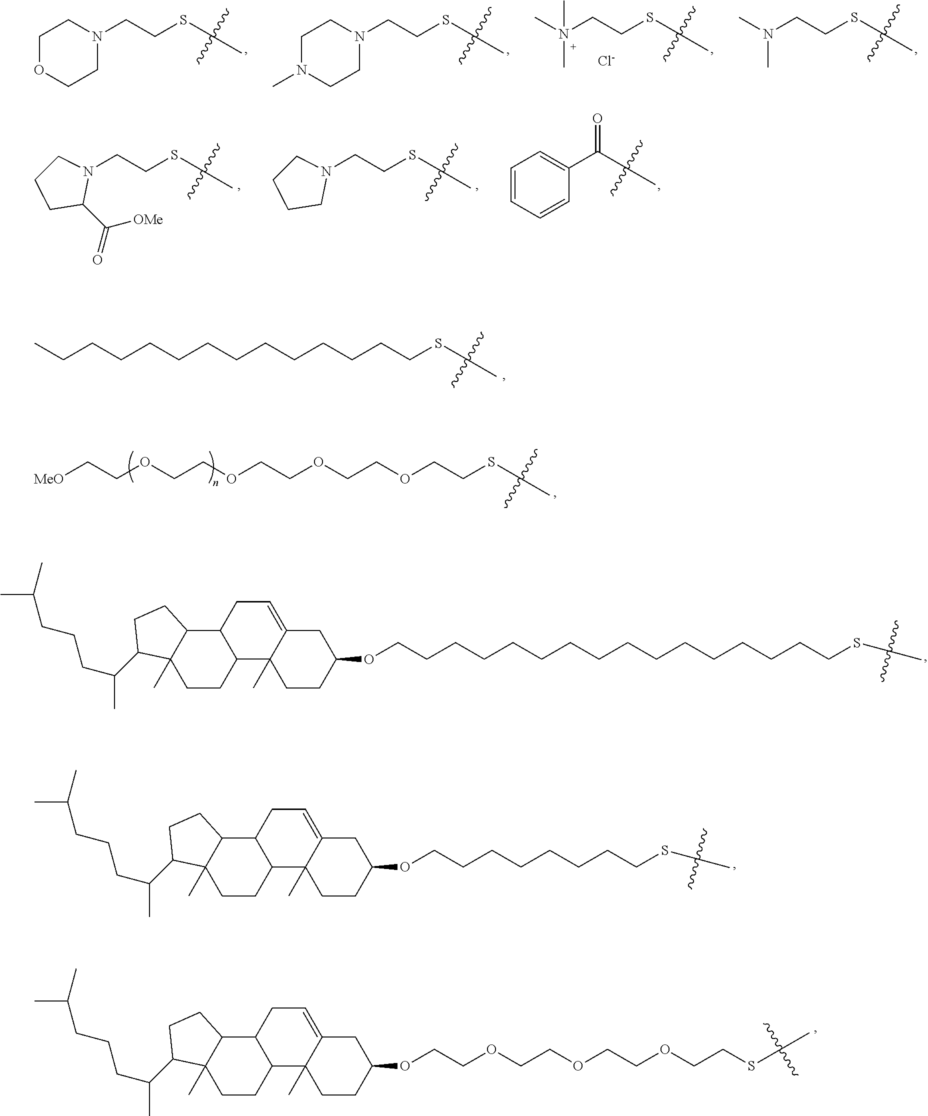

—S—(C₁-C₁₀ aliphatic) or —S—(C₁-C₅₀ aliphatic).

In some embodiments, L is a covalent bond and -L-R¹ is R².

In some embodiments, -L-R¹ is not hydrogen.

In some embodiments, —X-L-R¹ is R¹ is

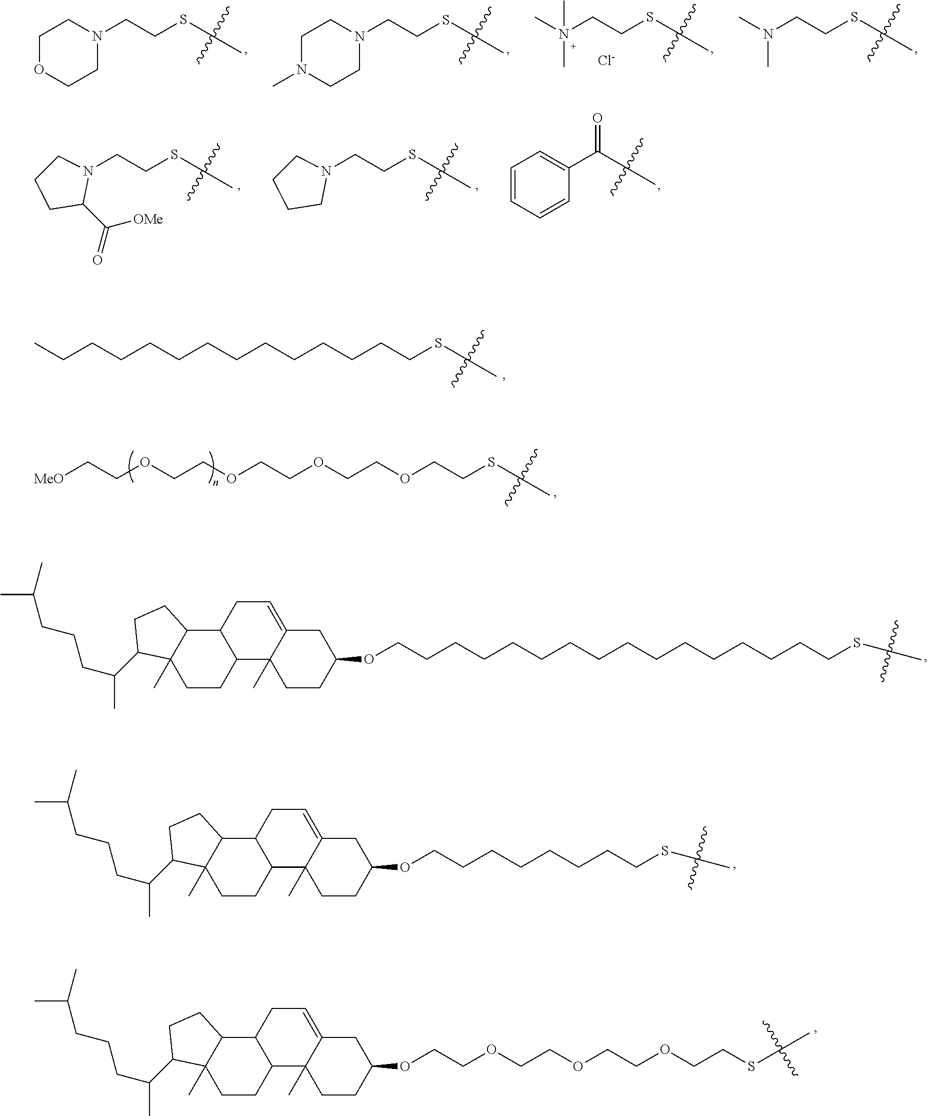

—S—(C₁-C₁₀ aliphatic) or —S—(C₁-C₅₀ aliphatic).

In some embodiments, —X-L-R¹ has the structure of

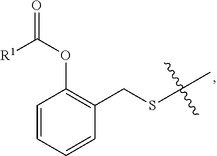

wherein the

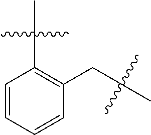 [SMILES: *Cc1ccccc1*]

moiety is optionally substituted. In some embodiments, —X-L-R¹ is

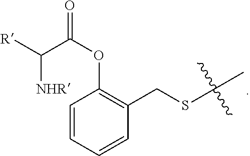

In some embodiments, —X-L-R¹ is

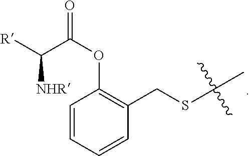

In some embodiments, —X-L-R¹ is

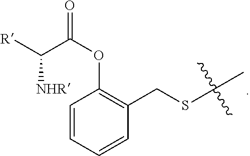

In some embodiments, —X-L-R¹ has the structure of

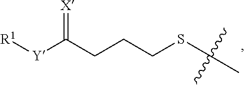

wherein X′ is O or S, Y′ is —O—, —S— or —NR′—, and the

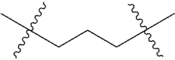 [SMILES: *CCC*]

moiety is optionally substituted. In some embodiments, Y′ is —O—, —S— or —NH—. In some embodiments,

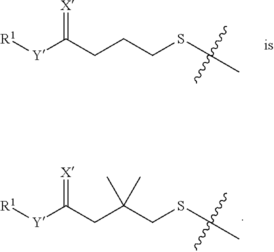

In some embodiments,

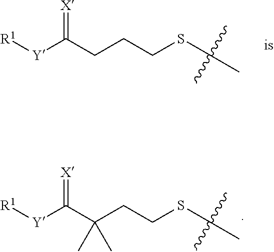

In some embodiments,

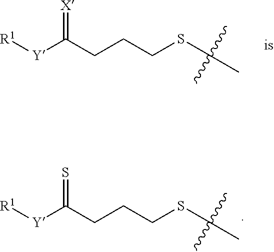

In some embodiments, —X-L-R¹ has the structure of

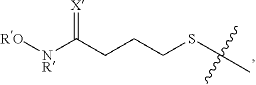

wherein X′ is O or S, and the

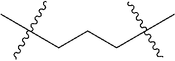 [SMILES: *CCC*]

moiety is optionally substituted. In some embodiments,

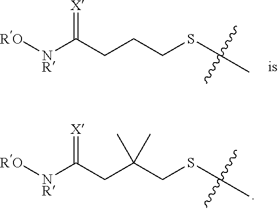

In some embodiments, —X-L-R¹ is

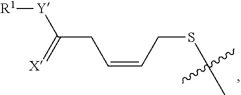

wherein the

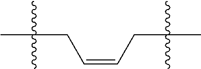 [SMILES: *C/C=C\C*]

is optionally substituted. In some embodiments, —X-L-R¹ is

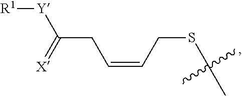

wherein the

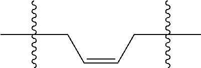 [SMILES: *C/C=C\C*]

is substituted. In some embodiments, —X-L-R¹ is

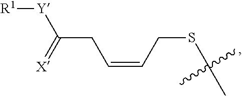

wherein the

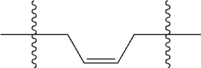 [SMILES: *C/C=C\C*]

is unsubstituted.

In some embodiments, —X-L-R¹ is R¹—C(O)—S-L^{x}-S—, wherein L^{x} is an optionally substituted group selected from

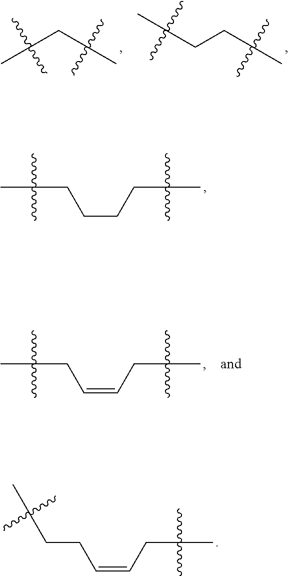

In some embodiments, L^{x} is

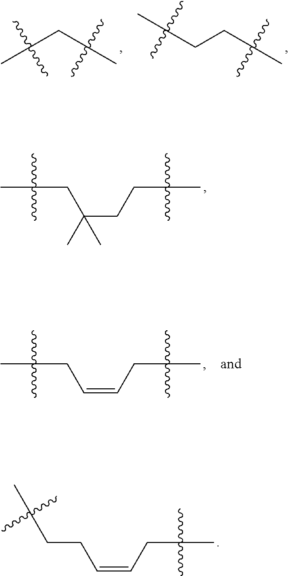

In some embodiments, —X-L-R¹ is (CH₃)₃C—S—S-L^{x}-S—. In some embodiments, —X-L-R¹ is R¹—C(═X′)—Y′—C(R)₂—S-L^{x}-S—. In some embodiments, —X-L-R¹ is R—C(═X′)—Y′—CH₂—S-L^{x}-S—. In some embodiments, —X-L-R¹ is

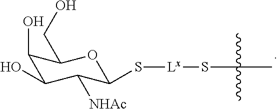

As will be appreciated by a person skilled in the art, many of the —X-L-R¹ groups described herein are cleavable and can be converted to —X— after administration to a subject. In some embodiments, —X-L-R¹ is cleavable. In some embodiments, —X-L-R¹ is —S-L-R¹, and is converted to —S⁻ after administration to a subject. In some embodiments, the conversion is promoted by an enzyme of a subject. As appreciated by a person skilled in the art, methods of determining whether the —S-L-R¹ group is converted to —S⁻ after administration is widely known and practiced in the art, including those used for studying drug metabolism and pharmacokinetics.

In some embodiments, the internucleotidic linkage having the structure of formula I is

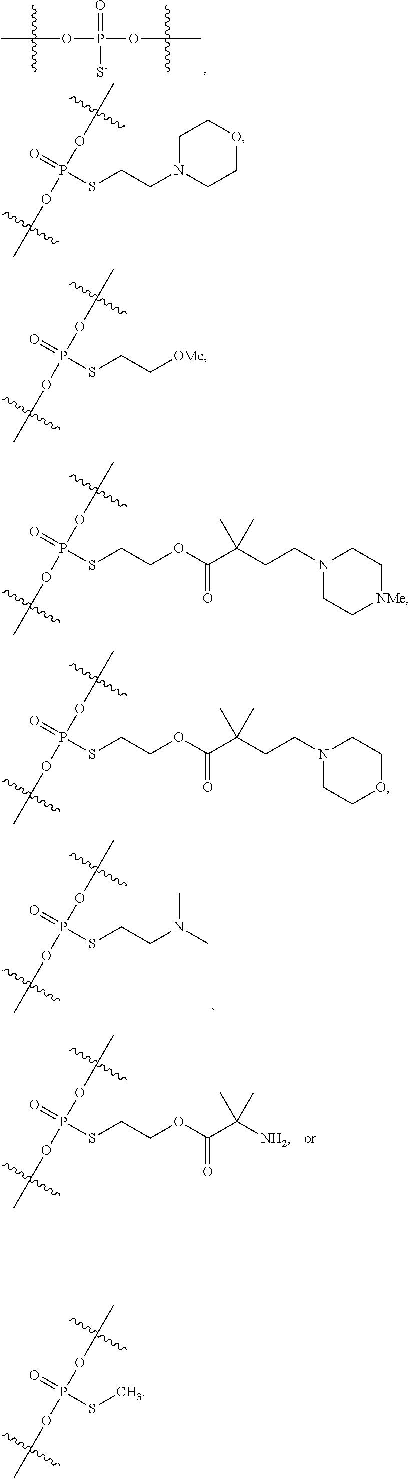

In some embodiments, the internucleotidic linkage of formula I has the structure of formula I-a:

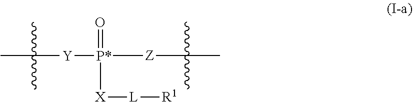

wherein each variable is independently as defined above and described herein.

In some embodiments, the internucleotidic linkage of formula I has the structure of formula I-b:

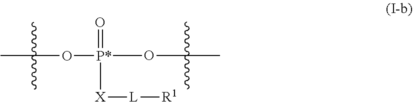

wherein each variable is independently as defined above and described herein.

In some embodiments, the internucleotidic linkage of formula I is an phosphorothioate triester linkage having the structure of formula I-c:

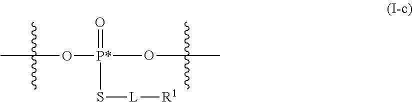

wherein:
P* is an asymmetric phosphorus atom and is either Rp or Sp;
L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, or heterocyclylene;
each R is independently hydrogen, or an optionally substituted group selected from C₁-C₆ aliphatic, phenyl, carbocyclyl, aryl, heteroaryl, or heterocyclyl;
each [symbol] independently represents a connection to a nucleoside; and
R¹ is not —H when L is a covalent bond.

In some embodiments, the internucleotidic linkage having the structure of formula I is

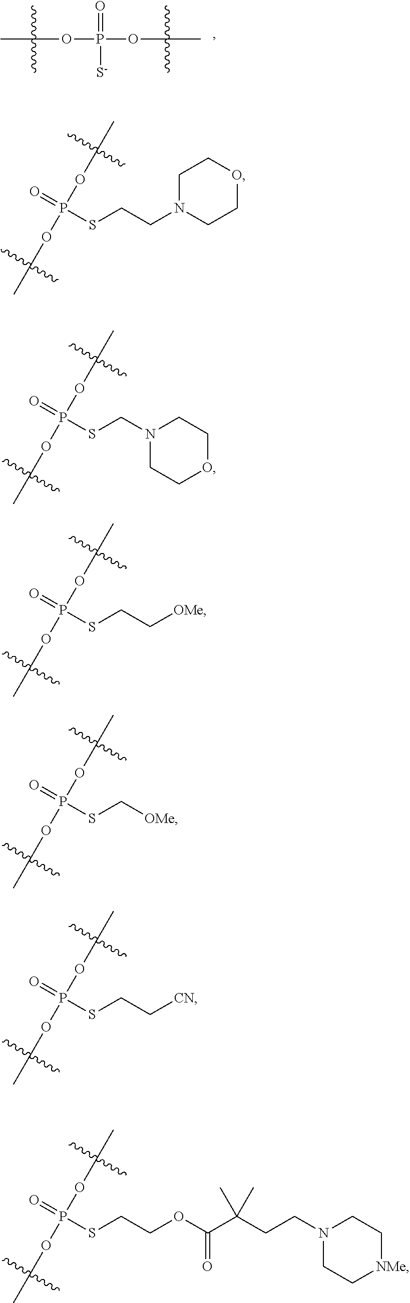

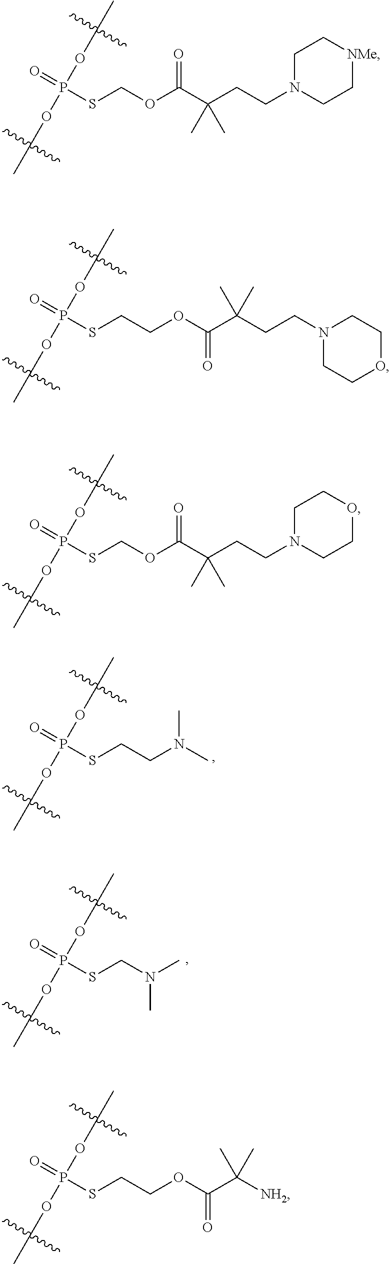 [SMILES: *OP(=O)(O*)SCOC(=O)C(C)(C)CCN1CCN(C)CC1 ; *OP(=O)(O*)SCCOC(=O)C(C)(C)CCN1CCOCC1 ; *OP(=O)(O*)SCOC(=O)C(C)(C)CCN1CCOCC1 ; *OP(=O)(O*)SCCN(C)C ; *OP(=O)(O*)SCN(C)C ; *OP(=O)(O*)SCCOC(=O)C(C)(C)N]

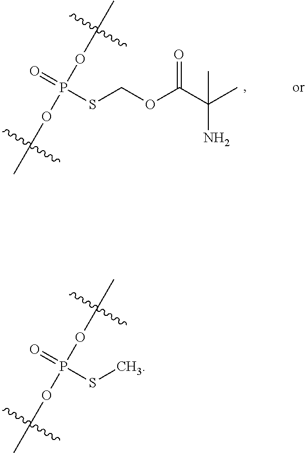 [SMILES: *OP(=O)(O*)SCOC(=O)C(C)(C)N ; *OP(=O)(O*)SC]

In some embodiments, the internucleotidic linkage having the structure of formula I-c is

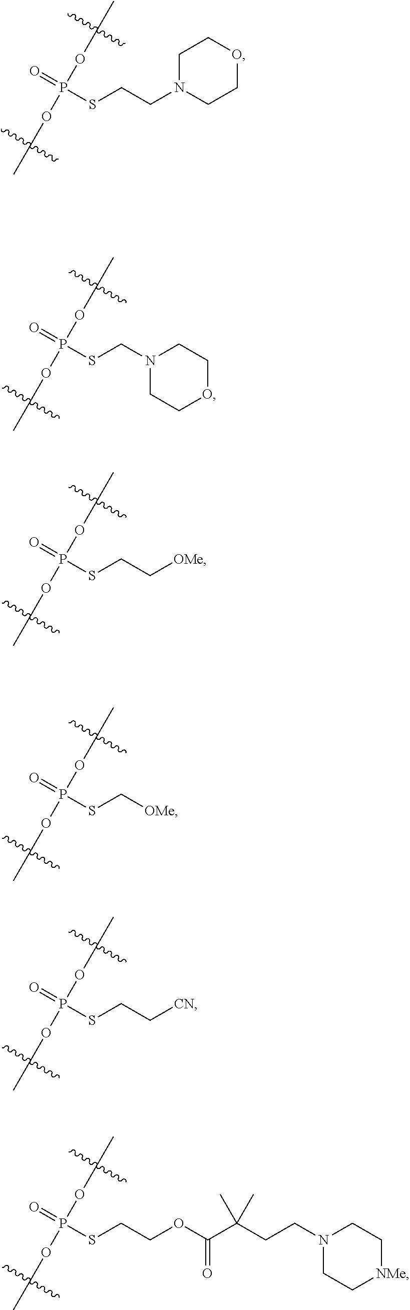 [SMILES: *OP(=O)(O*)SCCN1CCOCC1 ; *OP(=O)(O*)SCN1CCOCC1 ; *OP(=O)(O*)SCCOC ; *OP(=O)(O*)SCOC ; *OP(=O)(O*)SCCC#N ; *OP(=O)(O*)SCCOC(=O)C(C)(C)CCN1CCN(C)CC1]

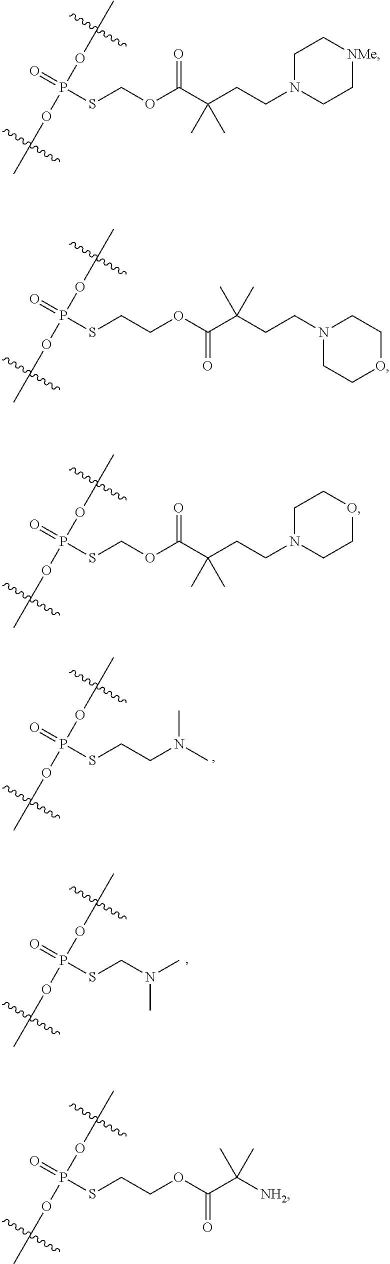 [SMILES: *OP(=O)(O*)SCOC(=O)C(C)(C)CCN1CCN(C)CC1 ; *OP(=O)(O*)SCCOC(=O)C(C)(C)CCN1CCOCC1 ; *OP(=O)(O*)SCOC(=O)C(C)(C)CCN1CCOCC1 ; *OP(=O)(O*)SCCN(C)C ; *OP(=O)(O*)SCN(C)C ; *OP(=O)(O*)SCCOC(=O)C(C)(C)N]

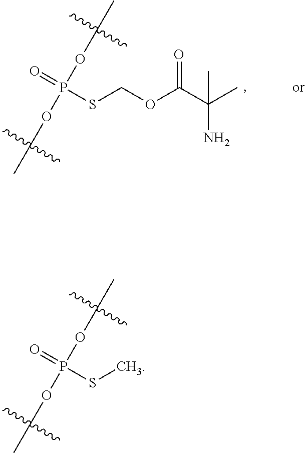 [SMILES: *OP(=O)(O*)SCOC(=O)C(C)(C)N ; *OP(=O)(O*)SC]

In some embodiments, the present disclosure provides a chirally controlled oligonucleotide comprising one or more phosphate diester linkages, and one or more modified internucleotide linkages having the formula of I-a, I-b, or I-c.

In some embodiments, a modified internucleotidic linkage has the structure of I. In some embodiments, a modified internucleotidic linkage has the structure of I-a. In some embodiments, a modified internucleotidic linkage has the structure of I-b. In some embodiments, a modified internucleotidic linkage has the structure of I-c.

In some embodiments, a modified internucleotidic linkage is phosphorothioate. Examples of internucleotidic linkages having the structure of formula I are widely known in the art, including but not limited to those described in US 20110294124, US 20120316224, US 20140194610, US 20150211006, US 20150197540, WO 2015107425, PCT/US2016/043542, and PCT/US2016/043598, each of which is incorporated herein by reference.

Non-limiting examples of internucleotidic linkages also include those described in the art, including, but not limited to, those described in any of: Gryaznov, S.; Chen, J.-K. J. Am. Chem. Soc. 1994, 116, 3143, Jones et al. J. Org. Chem. 1993, 58, 2983, Koshkin et al. 1998 Tetrahedron 54: 3607-3630, Lauritsen et al. 2002 Chem. Comm. 5: 530-531, Lauritsen et al. 2003 Bioo. Med. Chem. Lett. 13: 253-256, Mesmaeker et al. Angew. Chem., Int. Ed. Engl. 1994, 33, 226, Petersen et al. 2003 TRENDS Biotech. 21: 74-81, Schultz et al. 1996 Nucleic Acids Res. 24: 2966, Ts'o et al. Ann. N. Y. Acad. Sci. 1988, 507, 220, and Vasseur et al. J. Am. Chem. Soc. 1992, 114, 4006.

In some embodiments, provided oligonucleotides in provided compositions, e.g., oligonucleotides of a first plurality, comprise base modifications, sugar modifications, and/or internucleotidic linkage modifications, wherein one or more modifications is enrichment of deuterium. In some embodiments, e.g., an oligonucleotide is deuterated at one or more of its sugars, nucleobases, internucleotidic linkages, lipid moieties, linker moieties, targeting components, etc. Such oligonucleotides can be used in any composition or method described herein.

Oligonucleotides of the provided technologies can be of various lengths. In some embodiments, provided oligonucleotides comprise 5, 6, 7, 8, 9, 10, 11, 12, 13, 14, 15, 16, 17, 18, 19, 20, 21, 22, 23, 24, 25, 26, 27, 28, 29, 30, 40, 50 or more bases. In some embodiments, provided oligonucleotides comprise 10, 11, 12, 13, 14, 15, 16, 17, 18, 19, 20, 21, 22, 23, 24, 25, 26, 27, 28, 29, 30, 40, 50 or more bases. In some embodiments, provided oligonucleotides comprise 15, 16, 17, 18, 19, 20, 21, 22, 23, 24, 25, 26, 27, 28, 29, 30, 40, 50 or more bases. In some embodiments, provided oligonucleotides comprise 15 or more bases. In some embodiments, provided oligonucleotides comprise 16 or more bases. In some embodiments, provided oligonucleotides comprise 17 or more bases. In some embodiments, provided oligonucleotides comprise 18 or more bases. In some embodiments, provided oligonucleotides comprise 19 or more bases. In some embodiments, provided oligonucleotides comprise 20 or more bases. In some embodiments, provided oligonucleotides comprise 21 or more bases. In some embodiments, provided oligonucleotides comprise 22 or more bases. In some embodiments, provided oligonucleotides comprise 23 or more bases. In some embodiments, provided oligonucleotides comprise 24 or more bases. In some embodiments, provided oligonucleotides comprise 25 or more bases. In some embodiments, provided oligonucleotides comprise 26 or more bases. In some embodiments, provided oligonucleotides comprise 27 or more bases. In some embodiments, provided oligonucleotides comprise 28 or more bases. In some embodiments, provided oligonucleotides comprise 29 or more bases. In some embodiments, provided oligonucleotides comprise 30 or more bases. In some embodiments, provided oligonucleotides comprise 40 or more bases. In some embodiments, provided oligonucleotides comprise 50 or more bases. In some embodiments, provided oligonucleotides are 15mers. In some embodiments, provided oligonucleotides are 16mers. In some embodiments, provided oligonucleotides are 17mers. In some embodiments, provided oligonucleotides are 18mers. In some embodiments, provided oligonucleotides are 19mers. In some embodiments, provided oligonucleotides are 20mers. In some embodiments, provided oligonucleotides are 21mers. In some embodiments, provided oligonucleotides are 22mers. In some embodiments, provided oligonucleotides are 23mers. In some embodiments, provided oligonucleotides are 24mers. In some embodiments, provided oligonucleotides are 25mers. In some embodiments, provided oligonucleotides are 26mers. In some embodiments, provided oligonucleotides are 27mers. In some embodiments, provided oligonucleotides are 28mers. In some embodiments, provided oligonucleotides are 29mers. In some embodiments, provided oligonucleotides are 30mers.

In some embodiments, the present disclosure provides a chirally controlled oligonucleotide comprising at least one phosphate diester internucleotidic linkage and at least one phosphorothioate triester linkage having the structure of formula I-c. In some embodiments, the present disclosure provides a chirally controlled oligonucleotide comprising at least one phosphate diester internucleotidic linkage and at least two phosphorothioate triester linkages having the structure of formula I-c. In some embodiments, the present disclosure provides a chirally controlled oligonucleotide comprising at least one phosphate diester internucleotidic linkage and at least three phosphorothioate triester linkages having the structure of formula I-c. In some embodiments, the present disclosure provides a chirally controlled oligonucleotide comprising at least one phosphate diester internucleotidic linkage and at least four phosphorothioate triester linkages having the structure of formula I-c. In some embodiments, the present disclosure provides a chirally controlled oligonucleotide comprising at least one phosphate diester internucleotidic linkage and at least five phosphorothioate triester linkages having the structure of formula I-c.

In some embodiments, a chirally controlled oligonucleotide is designed such that one or more nucleotides comprise a phosphorus modification prone to “autorelease” under certain conditions. That is, under certain conditions, a particular phosphorus modification is designed such that it self-cleaves from the oligonucleotide to provide, e.g., a phosphate diester such as those found in naturally occurring DNA and RNA. In some embodiments, such a phosphorus modification has a structure of —O-L-R¹, wherein each of L and R¹ is independently as defined above and described herein. In some embodiments, an autorelease group comprises a morpholino group. In some embodiments, an autorelease group is characterized by the ability to deliver an agent to the internucleotidic phosphorus linker, which agent facilitates further modification of the phosphorus atom such as, e.g., desulfurization. In some embodiments, the agent is water and the further modification is hydrolysis to form a phosphate diester as is found in naturally occurring DNA and RNA.

In some embodiments, a chirally controlled oligonucleotide is designed such that the resulting pharmaceutical properties are improved through one or more particular modifications at phosphorus. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). For instance, Vives et al., (Nucleic Acids Research (1999), 27(20):4071-76) found that tert-butyl SATE pro-oligonucleotides displayed markedly increased cellular penetration compared to the parent oligonucleotide.

In some embodiments, a modification at a linkage phosphorus is characterized by its ability to be transformed to a phosphate diester, such as those present in naturally occurring DNA and RNA, by one or more esterases, nucleases, and/or cytochrome P450 enzymes, including but not limited to, those listed below:

| Family | Gene |
|---|---|
| CYP1 | CYP1A1, CYP1A2, CYP1B1 |
| CYP2 | CYP2A6, CYP2A7, CYP2A13, CYP2B6, |
| | CYP2C8, CYP2C9, CYP2C18, CYP2C19, |
| | CYP2D6, CYP2E1, CYP2F1, CYP2J2, |
| | CYP2R1, CYP2S1, CYP2U1, CYP2W1 |
| CYP3 | CYP3A4, CYP3A5, CYP3A7, CYP3A43 |
| CYP4 | CYP4A11, CYP4A22, CYP4B1, CYP4F2, |
| | CYP4F3, CYP4F8, CYP4F11, CYP4F12, |
| | CYP4F22, CYP4V2, CYP4X1, CYP4Z1 |
| CYP5 | CYP5A1 |
| CYP7 | CYP7A1, CYP7B1 |
| CYP8 | CYP8A1 (prostacyclin synthase), CYP8B1 |
| | (bile acid biosynthesis) |
| CYP11 | CYP11A1, CYP11B1, CYP11B2 |
| CYP17 | CYP17A1 |
| CYP19 | CYP19A1 |
| CYP20 | CYP20A1 |
| CYP21 | CYP21A2 |
| CYP24 | CYP24A1 |
| CYP26 | CYP26A1, CYP26B1, CYP26C1 |
| CYP27 | CYP27A1 (bile acid biosynthesis), CYP27B1 |
| | (vitamin D3 1-alpha hydroxylase, activates |
| | vitamin D3), CYP27C1 (unknown function) |
| CYP39 | CYP39A1 |
| CYP46 | CYP46A1 |
| CYP51 | CYP51A1 (lanosterol 14-alpha demethylase) |

In some embodiments, a modification at phosphorus results in a P-modification moiety characterized in that it acts as a pro-drug, e.g., the P-modification moiety facilitates delivery of an oligonucleotide to a desired location prior to removal. For instance, in some embodiments, a P-modification moiety results from PEGylation at the linkage phosphorus. One of skill in the relevant arts will appreciate that various PEG chain lengths are useful and that the selection of chain length will be determined in part by the result that is sought to be achieved by PEGylation. For instance, in some embodiments, PEGylation is effected in order to reduce RES uptake and extend in vivo circulation lifetime of an oligonucleotide.

In some embodiments, a PEGylation reagent for use in accordance with the present disclosure is of a molecular weight of about 300 g/mol to about 100,000 g/mol. In some embodiments, a PEGylation reagent is of a molecular weight of about 300 g/mol to about 10,000 g/mol. In some embodiments, a PEGylation reagent is of a molecular weight of about 300 g/mol to about 5,000 g/mol. In some embodiments, a PEGylation reagent is of a molecular weight of about 500 g/mol. In some embodiments, a PEGylation reagent of a molecular weight of about 1000 g/mol. In some embodiments, a PEGylation reagent is of a molecular weight of about 3000 g/mol. In some embodiments, a PEGylation reagent is of a molecular weight of about 5000 g/mol.

In certain embodiments, a PEGylation reagent is PEG500. In certain embodiments, a PEGylation reagent is PEG1000. In certain embodiments, a PEGylation reagent is PEG3000. In certain embodiments, a PEGylation reagent is PEG5000.

In some embodiments, a P-modification moiety is characterized in that it acts as a PK enhancer, e.g., lipids, PEGylated lipids, etc.

In some embodiments, a P-modification moiety is characterized in that it acts as an agent which promotes cell entry and/or endosomal escape, such as a membrane-disruptive lipid or peptide.

In some embodiments, a P-modification moiety is characterized in that it acts as a targeting agent. In some embodiments, a P-modification moiety is or comprises a targeting agent. The phrase “targeting agent,” as used herein, is an entity that is associates with a payload of interest (e.g., with an oligonucleotide or oligonucleotide composition) and also interacts with a target site of interest so that the payload of interest is targeted to the target site of interest when associated with the targeting agent to a materially greater extent than is observed under otherwise comparable conditions when the payload of interest is not associated with the targeting agent. A targeting agent may be, or comprise, any of a variety of chemical moieties, including, for example, small molecule moieties, nucleic acids, polypeptides, carbohydrates, etc. Targeting agents are described further by Adarsh et al., “Organelle Specific Targeted Drug Delivery—A Review,” International Journal of Research in Pharmaceutical and Biomedical Sciences, 2011, p. 895.

Example such targeting agents include, but are not limited to, proteins (e.g. Transferrin), oligopeptides (e.g., cyclic and acylic RGD-containing oligopedptides), antibodies (monoclonal and polyclonal antibodies, e.g. IgG, IgA, IgM, IgD, IgE antibodies), sugars/carbohydrates (e.g., monosaccharides and/or oligosaccharides (mannose, mannose-6-phosphate, galactose, and the like)), vitamins (e.g., folate), or other small biomolecules. In some embodiments, a targeting moiety is a steroid molecule (e.g., bile acids including cholic acid, deoxycholic acid, dehydrocholic acid; cortisone; digoxigenin; testosterone; cholesterol; cationic steroids such as cortisone having a trimethylaminomethyl hydrazide group attached via a double bond at the 3-position of the cortisone ring, etc.). In some embodiments, a targeting moiety is a lipophilic molecule (e.g., alicyclic hydrocarbons, saturated and unsaturated fatty acids, waxes, terpenes, and polyalicyclic hydrocarbons such as adamantine and buckminsterfullerenes). In some embodiments, a lipophilic molecule is a terpenoid such as vitamin A, retinoic acid, retinal, or dehydroretinal. In some embodiments, a targeting moiety is a peptide.

In some embodiments, a P-modification moiety is a targeting agent of formula —X-L-R¹ wherein each of X, L, and R¹ are as defined in Formula I above.

In some embodiments, a P-modification moiety is characterized in that it facilitates cell specific delivery.

In some embodiments, a P-modification moiety is characterized in that it falls into one or more of the above-described categories. For instance, in some embodiments, a P-modification moiety acts as a PK enhancer and a targeting ligand. In some embodiments, a P-modification moiety acts as a pro-drug and an endosomal escape agent. One of skill in the relevant arts would recognize that numerous other such combinations are possible and are contemplated by the present disclosure.

In some embodiments, a carbocyclyl, aryl, heteroaryl, or heterocyclyl group, or a bivalent or polyvalent group thereof, is a C₃-C₃₀ carbocyclyl, aryl, heteroaryl, or heterocyclyl group, or a bivalent and/or polyvalent group thereof.

### Nucleobases

In some embodiments, a nucleobase present in a provided oligonucleotide is a natural nucleobase or a modified nucleobase derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Example modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a modified base is optionally substituted adenine, cytosine, guanine, thymine, or uracil. In some embodiments, a modified nucleobase is independently adenine, cytosine, guanine, thymine or uracil, modified by one or more modifications by which:
(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
(2) one or more atoms of a nucleobase are independently replaced with a different atom selected from carbon, nitrogen or sulfur;
(3) one or more double bonds in a nucleobase are independently hydrogenated; or
(4) one or more aryl or heteroaryl rings are independently inserted into a nucleobase.

Structures represented by the following general formulae are also contemplated as modified nucleobases:

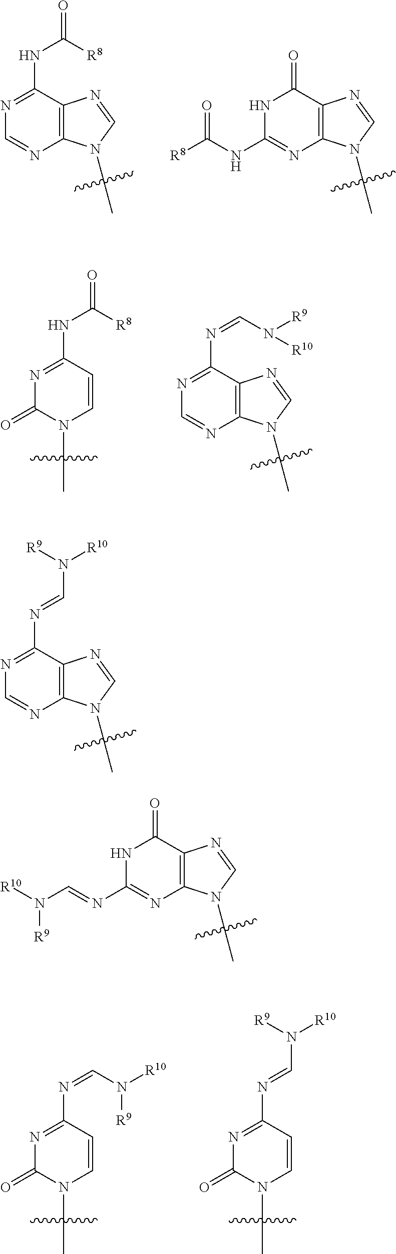

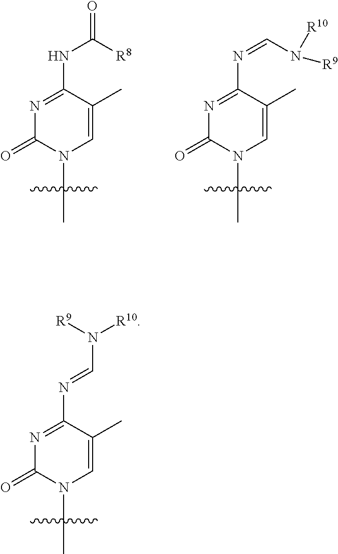

wherein R⁸ is an optionally substituted, linear or branched group selected from aliphatic, aryl, aralkyl, aryloxylalkyl, carbocyclyl, heterocyclyl or heteroaryl group having 1 to 15 carbon atoms, including, by way of example only, a methyl, isopropyl, phenyl, benzyl, or phenoxymethyl group; and each of R⁹ and R¹⁰ is independently an optionally substituted group selected from linear or branched aliphatic, carbocyclyl, aryl, heterocyclyl and heteroaryl.

Modified nucleobases also include expanded-size nucleobases in which one or more aryl rings, such as phenyl rings, have been added. Nucleic base replacements described in the Glen Research catalog (www.glenresearch.com); Krueger A T et al, Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for the synthesis of the nucleic acids described herein. Some examples of these expanded-size nucleobases are shown below:

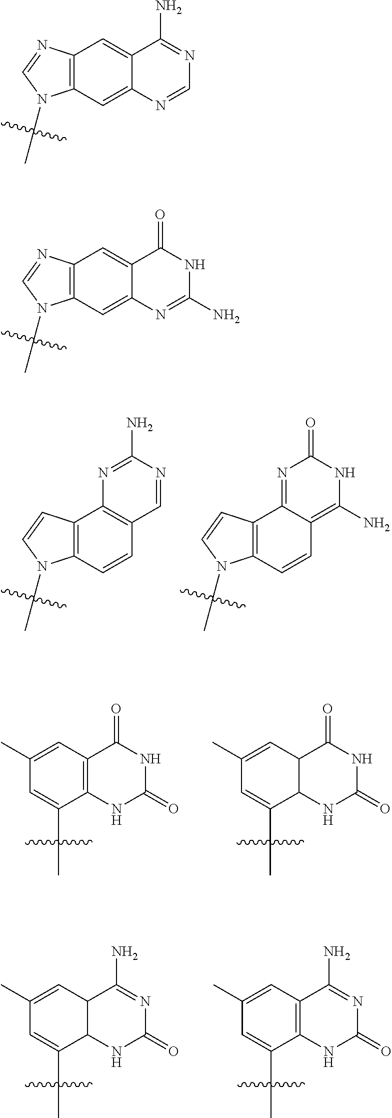

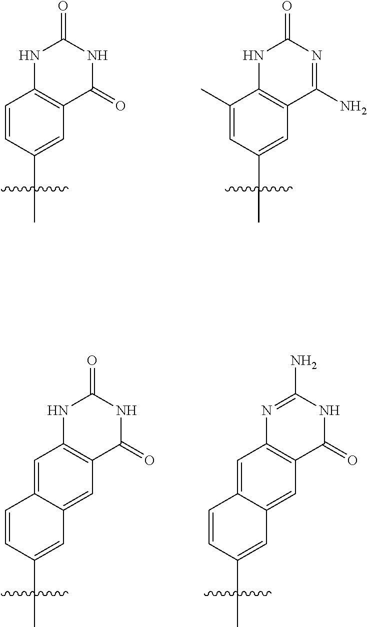 [SMILES: *c1ccc2[nH]c(=O)[nH]c(=O)c2c1 ; *c1cc(C)c2[nH]c(=O)nc(N)c2c1 ; *c1ccc2cc3[nH]c(=O)[nH]c(=O)c3cc2c1 ; *c1ccc2cc3nc(N)[nH]c(=O)c3cc2c1]

Herein, modified nucleobases also encompass structures that are not considered nucleobases but are other moieties such as, but not limited to, corrin- or porphyrin-derived rings. Porphyrin-derived base replacements have been described in Morales-Rojas, H and Kool, E T, Org. Lett., 2002, 4, 4377-4380. Shown below is an example of a porphyrin-derived ring which can be used as a base replacement:

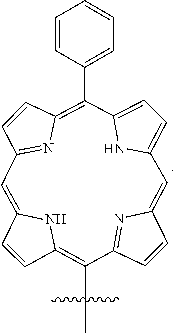 [SMILES: *c1c2nc(cc3ccc([nH]3)c(-c3ccccc3)c3nc(cc4ccc1[nH]4)C=C3)C=C2]

In some embodiments, modified nucleobases are of any one of the following structures, optionally substituted:

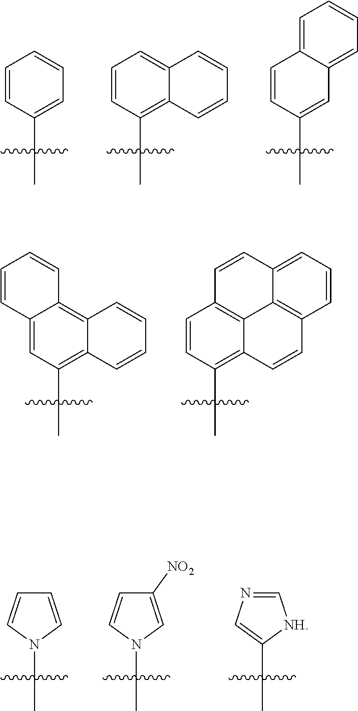

In some embodiments, a modified nucleobase is fluorescent. Example such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

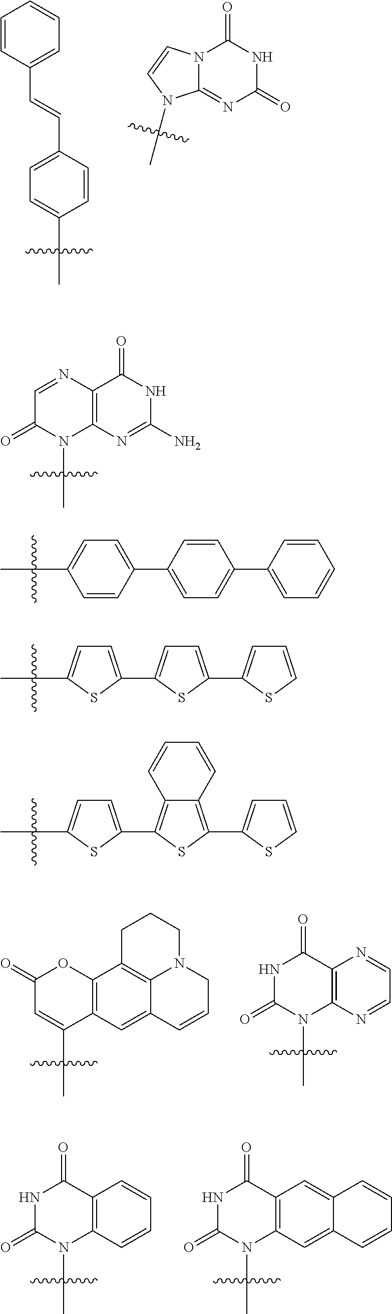

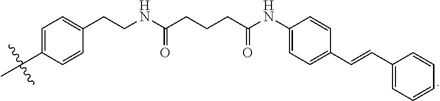 [SMILES: *c1ccc(CCNC(=O)CCCC(=O)Nc2ccc(/C=C/c3ccccc3)cc2)cc1]

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, other nucleosides can also be used in the process disclosed herein and include nucleosides that incorporate modified nucleobases, or nucleobases covalently bound to modified sugars. Some examples of nucleosides or nucleotides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or (S)-chirality at the 6′-position and include the analogs described in U.S. Pat. No. 7,399,845. In other embodiments, nucleosides include 5′-modified bicyclic nucleoside analogs that have either (R) or (S)-chirality at the 5′-position and include the analogs described in US Patent Application Publication No. 20070287831.

In some embodiments, a nucleobase or modified nucleobase comprises one or more biomolecule binding moieties such as e.g., antibodies, antibody fragments, biotin, avidin, streptavidin, receptor ligands, or chelating moieties. In other embodiments, a nucleobase or modified nucleobase is 5-bromouracil, 5-iodouracil, or 2,6-diaminopurine. In some embodiments, a nucleobase or modified nucleobase is modified by substitution with a fluorescent or biomolecule binding moiety. In some embodiments, the substituent on a nucleobase or modified nucleobase is a fluorescent moiety. In some embodiments, the substituent on a nucleobase or modified nucleobase is biotin or avidin.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the modified nucleobases, sugars, and internucleotidic linkages of each of which are incorporated by reference.

In some embodiments, a base is optionally substituted A, T, C, G or U, wherein one or more —NH₂ are independently and optionally replaced with —C(-L-R′)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms. In some embodiments, a modified base is optionally substituted A, T, C, G or U, wherein one or more —NH₂ are independently and optionally replaced with —C(-L-R′)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R′), or ═C(-L-R′)₂, wherein two or more -L-R¹ are optionally taken together with their intervening atoms to form a 3-30 membered bicyclic or polycyclic ring having 0-10 heteroatom ring atoms, wherein the modified base is different than the natural A, T, C, G and U. In some embodiments, a base is optionally substituted A, T, C, G or U. In some embodiments, a modified base is substituted A, T, C, G or U, wherein the modified base is different than the natural A, T, C, G and U.

In some embodiments, a modified nucleotide or nucleotide analog is any modified nucleotide or nucleotide analog described in any of: Albaek et al. 2006 J. Org. Chem. 71: 7731-7740; Braasch et al., Chem. Biol., 2001, 8, 1-7; Chattopadhyaya et al. 2009 J. Org. Chem. 74: 18-134; Elayadi et al, Curr. Opinion Invens. Drugs, 2001, 2, 5561; Frieden et al. 2003 Nucl. Acids Res. 21: 6365-6372; Freier et al. 1997 Nucl. Acids Res. 25: 4429-4443; Gryaznov et al. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Leumann et al. 2002 Bioorg. Med. Chem. 10: 841-854; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Oram et al, Curr. Opinion Mol. Ther., 2001, 3, 239-243; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Singh et al. 1998 Chem. Commun. 4: 455-456; Sorensen 2003 Chem. Comm. 2130-2131; Srivastava et al. 2007 J. Am. Chem. Soc, 129: 8362-8379; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; Wahlestedt et al. 2000 Proc. Natl. Acad. Sci. U.S.A 97: 5633-5638; U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 6,268,490; 6,525,191; 6,670,461; 6,770,748; 6,794,499; 7,034,133; 7,053,207; 7,399,845; and 7,427,672; U.S. Patent Publication Nos. US2004/0171570; US2005/0130923; US2007/0287831; and US2008/0039618; U.S. patent application Ser. Nos. 12/129,154; 60/989,574; 61/026,995; 61/026,998; 61/056,564; 61/086,231; 61/097,787; and 61/099,844; PCT International Applications Nos. PCT/US2008/064591; PCT/US2008/066154; and PCT/US2008/068922. WO 2004/106356; WO 1994/14226; WO 2005/021570; WO 2007/134181; WO 2007/0900071; WO 2008/154401; WO2008/101157; WO2008/150729; WO2009/006478; or WO 2016/079181. Example nucleobases are also described in US 20110294124, US 20120316224, US 20140194610, US 20150211006, US 20150197540, WO 2015107425, PCT/US2016/043542, and PCT/US2016/043598, each of which is incorporated herein by reference.

### Sugars

In some embodiments, provided oligonucleotides comprise one or more modified sugar moieties.

The most common naturally occurring nucleotides are comprised of ribose sugars linked to the nucleobases adenosine (A), cytosine (C), guanine (G), and thymine (T) or uracil (U). Also contemplated are modified nucleotides wherein a phosphate group or linkage phosphorus in the nucleotides can be linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context. In some embodiments, nucleotides or modified nucleotides comprising an unprotected —OH moiety are used in accordance with methods of the present disclosure.

Other modified sugars can also be incorporated within a provided oligonucleotide. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the the 2′, 3′, 4′, 5′, or 6′ positions of the sugar or modified sugar, including the 3′ position of the sugar on the 3′-terminal nucleotide or in the 5′ position of the 5′-terminal nucleotide.

In some embodiments, a 2′-modification is 2′-F.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

Modified sugars also include locked nucleic acids (LNAs). In some embodiments, two substituents on sugar carbon atoms are taken together to form a bivalent moiety. In some embodiments, two substituents are on two different sugar carbon atoms. In some embodiments, a formed bivalent moiety has the structure of -L- as defined herein. In some embodiments, -L- is —O—CH₂—, wherein —CH₂— is optionally substituted. In some embodiments, -L- is —O—CH₂—. In some embodiments, -L- is —O—CH(Et)-. In some embodiments, -L- is between C₂ and C₄ of a sugar moiety. In some embodiments, a locked nucleic acid has the structure indicated below. A locked nucleic acid of the structure below is indicated, wherein Ba represents a nucleobase or modified nucleobase as described herein, and wherein R^{2s} is —OCH₂C4′-.

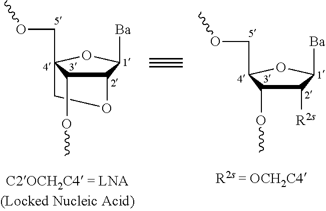

In some embodiments, a modified sugar is an ENA such as those described in, e.g., Seth et al., J Am Chem Soc. 2010 Oct. 27; 132(42): 14942-14950. In some embodiments, a modified sugar is any of those found in an XNA (xenonucleic acid), for instance, arabinose, anhydrohexitol, threose, 2′ fluoroarabinose, or cyclohexene.

Modified sugars include sugar mimetics such as cyclobutyl or cyclopentyl moieties in place of the pentofuranosyl sugar. Representative United States patents that teach the preparation of such modified sugar structures include, but are not limited to, U.S. Pat. Nos. 4,981,957; 5,118,800; 5,319,080; and 5,359,044. Some modified sugars that are contemplated include sugars in which the oxygen atom within the ribose ring is replaced by nitrogen, sulfur, selenium, or carbon. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc).

Non-limiting examples of modified sugars include glycerol, which form glycerol nucleic acid (GNA) analogues. One example of a GNA analogue is shown below and is described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603 (X═O⁻):

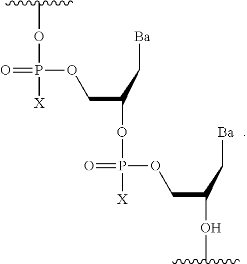

Another example of a GNA derived analogue, flexible nucleic acid (FNA) based on the mixed acetal aminal of formyl glycerol, is described in Joyce G F et al., PNAS, 1987, 84, 4398-4402 and Heuberger B D and Switzer C, J. Am. Chem. Soc., 2008, 130, 412-413, and is shown below:

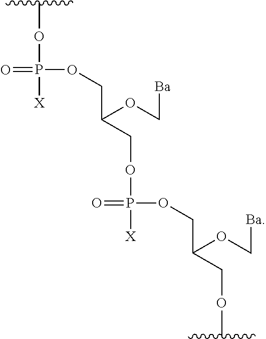

Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars. In some embodiments, a hexopyranosyl (6′ to 4′) sugar is of any one in the following formulae:

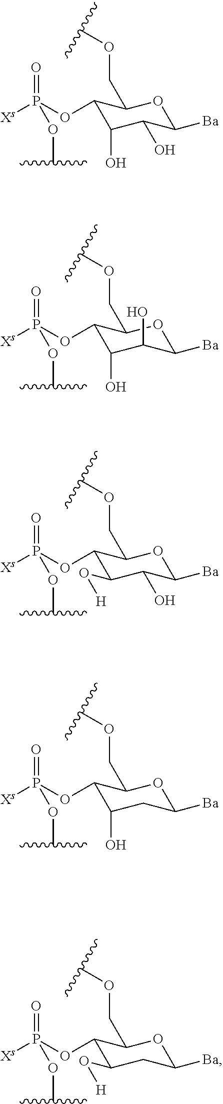

wherein X^{s} corresponds to the P-modification group “—XLR¹” described herein and Ba is as defined herein.

In some embodiments, a pentopyranosyl (4′ to 2′) sugar is of any one in the following formulae:

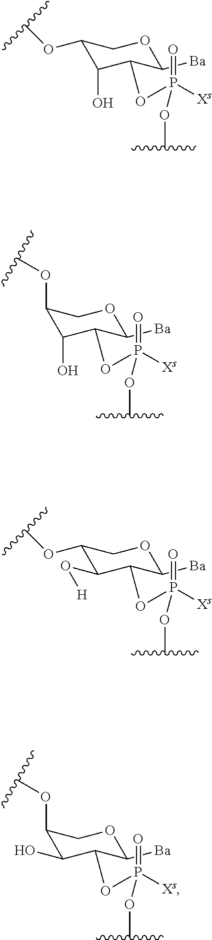

wherein X^{s} corresponds to the P-modification group “—XLR¹” described herein and Ba is as defined herein.

In some embodiments, a pentopyranosyl (4′ to 3′) sugar is of any one in the following formulae:

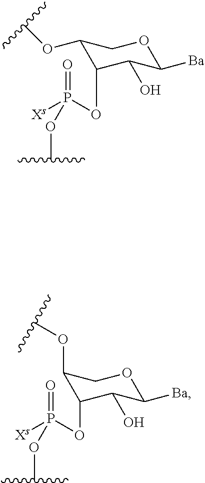

wherein X^{s} corresponds to the P-modification group “—XLR¹” described herein and Ba is as defined herein.

In some embodiments, a tetrofuranosyl (3′ to 2′) sugar is of either in the following formulae:

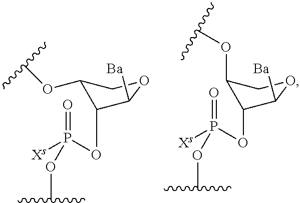

wherein X^{s} corresponds to the P-modification group “—XLR¹” described herein and Ba is as defined herein.

In some embodiments, a modified sugar is of any one in the following formulae:

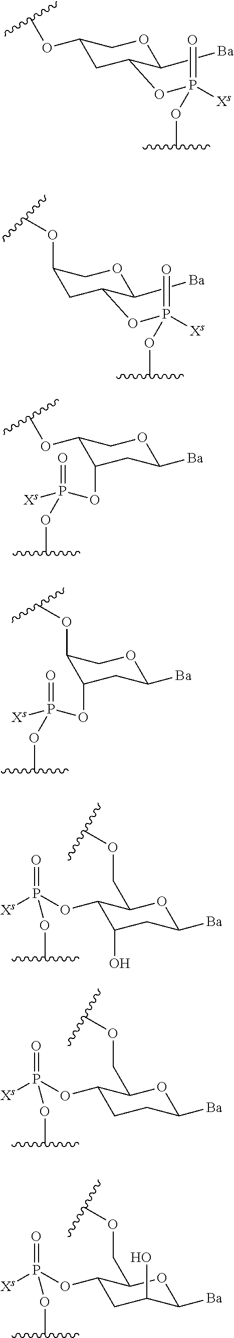

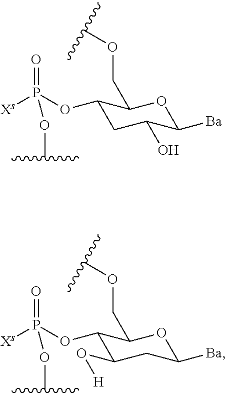

wherein X^{s} corresponds to the P-modification group “—XLR¹” described herein and Ba is as defined herein.

In some embodiments, one or more hydroxyl group in a sugar moiety is optionally and independently replaced with halogen, R′ —N(R′)₂, —OR′, or —SR′, wherein each R′ is independently as defined above and described herein.

In some embodiments, a sugar mimetic is as illustrated below, wherein X′ corresponds to the P-modification group “—XLR¹” described herein, Ba is as defined herein, and X¹ is selected from —S—, —Se—, —CH₂—, —NMe-, —NEt- or —NiPr—.

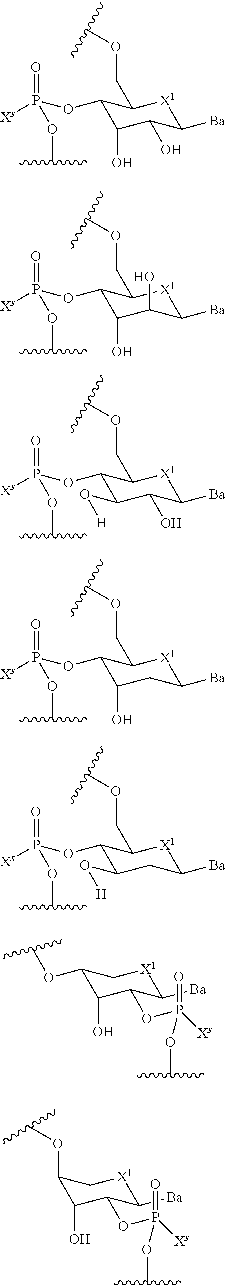

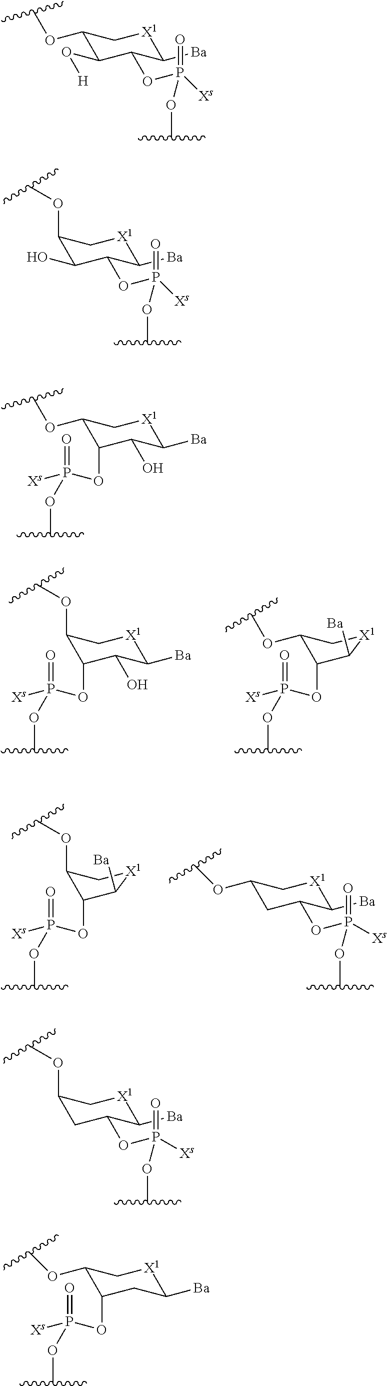

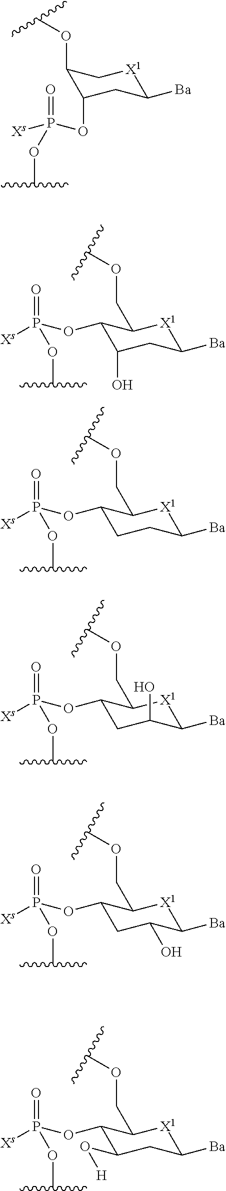

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more (e.g., 55%, 60%, 65%, 70%, 75%, 80%, 85%, 90%, 95% or more), inclusive, of the sugars in a chirally controlled oligonucleotide composition are modified. In some embodiments, only purine residues are modified (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more [e.g., 55%, 60%, 65%, 70%, 75%, 80%, 85%, 90%, 95% or more] of the purine residues are modified). In some embodiments, only pyrimidine residues are modified (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more [e.g., 55%, 60%, 65%, 70%, 75%, 80%, 85%, 90%, 95% or more] of the pyridimine residues are modified). In some embodiments, both purine and pyrimidine residues are modified.

Modified sugars and sugar mimetics can be prepared by methods known in the art, including, but not limited to: A. Eschenmoser, Science (1999), 284:2118; M. Bohringer et al, Helv. Chim. Acta (1992), 75:1416-1477; M. Egli et al, J. Am. Chem. Soc. (2006), 128(33):10847-56; A. Eschenmoser in Chemical Synthesis: Gnosis to Prognosis, C. Chatgilialoglu and V. Sniekus, Ed., (Kluwer Academic, Netherlands, 1996), p.293; K.-U. Schoning et al, Science (2000), 290:1347-1351; A. Eschenmoser et al, Helv. Chim. Acta (1992), 75:218; J. Hunziker et al, Helv. Chim. Acta (1993), 76:259; G. Otting et al, Helv. Chim. Acta (1993), 76:2701; K. Groebke et al, Helv. Chim. Acta (1998), 81:375; and A. Eschenmoser, Science (1999), 284:2118. Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310). In some embodiments, a modified sugar is any of those described in PCT Publication No. WO2012/030683, incorporated herein by reference, and/or depicted herein. In some embodiments, a modified sugar is any modified sugar described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 20070900071; WO 20070900071; or WO 2016/079181.

In some embodiments, a modified sugar moiety is an optionally substituted pentose or hexose moiety. In some embodiments, a modified sugar moiety is an optionally substituted pentose moiety. In some embodiments, a modified sugar moiety is an optionally substituted hexose moiety. In some embodiments, a modified sugar moiety is an optionally substituted ribose or hexitol moiety. In some embodiments, a modified sugar moiety is an optionally substituted ribose moiety. In some embodiments, a modified sugar moiety is an optionally substituted hexitol moiety.

In some embodiments, an example modified internucleotidic linkage and/or sugar is selected from those of:

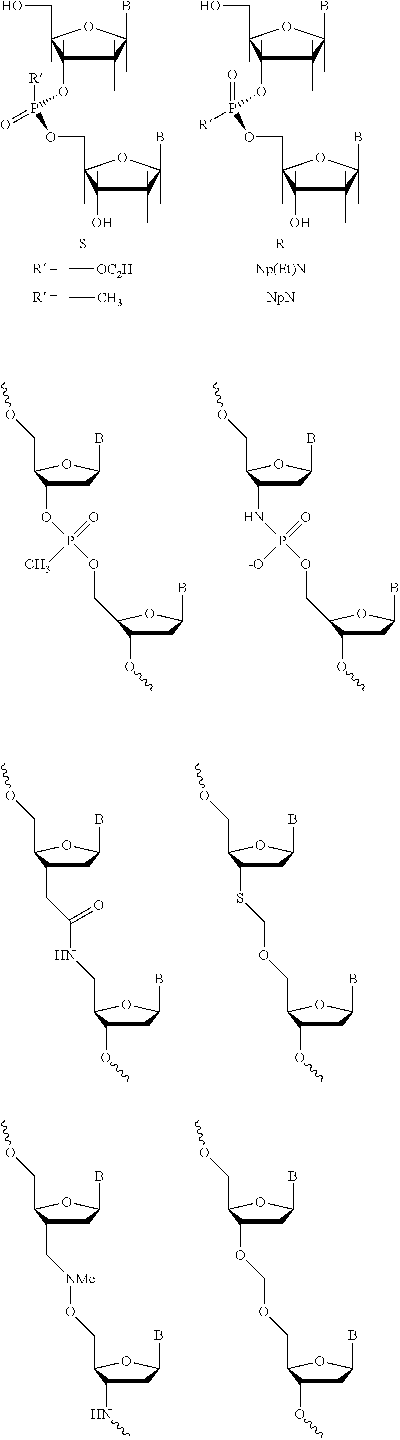

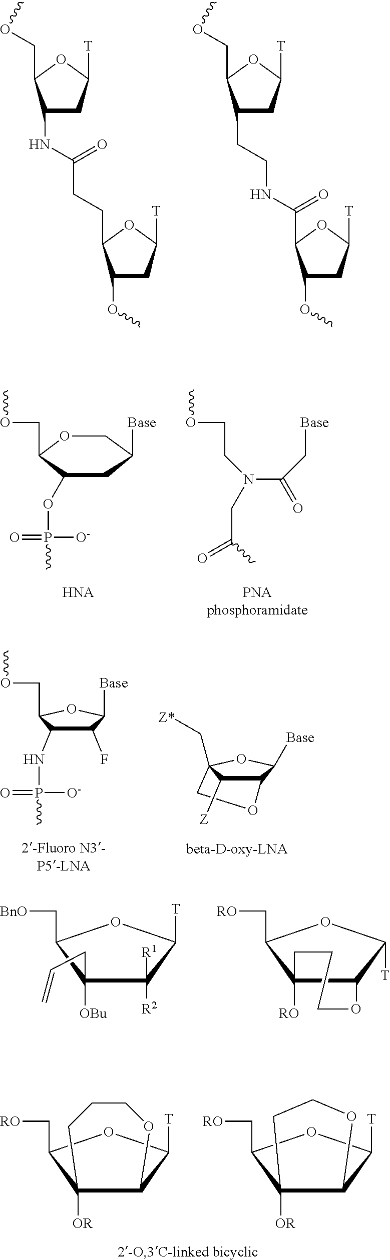

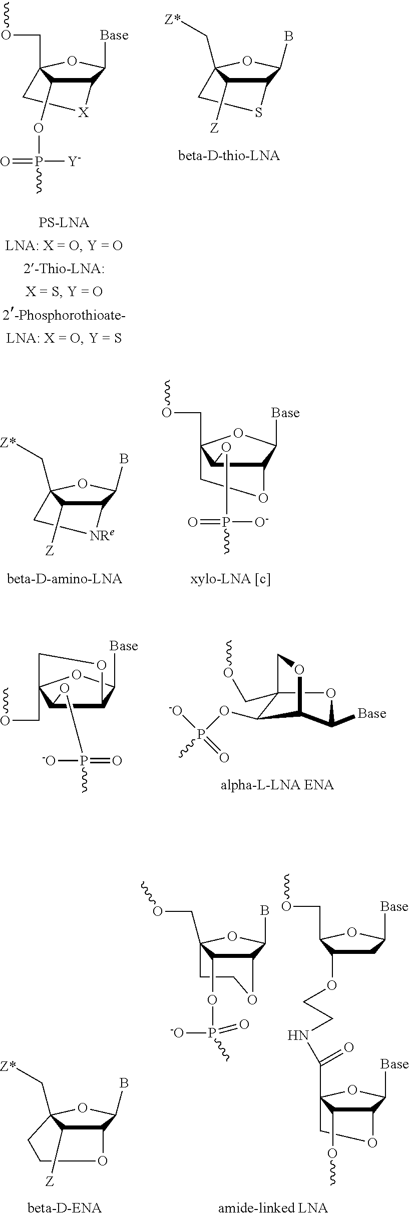

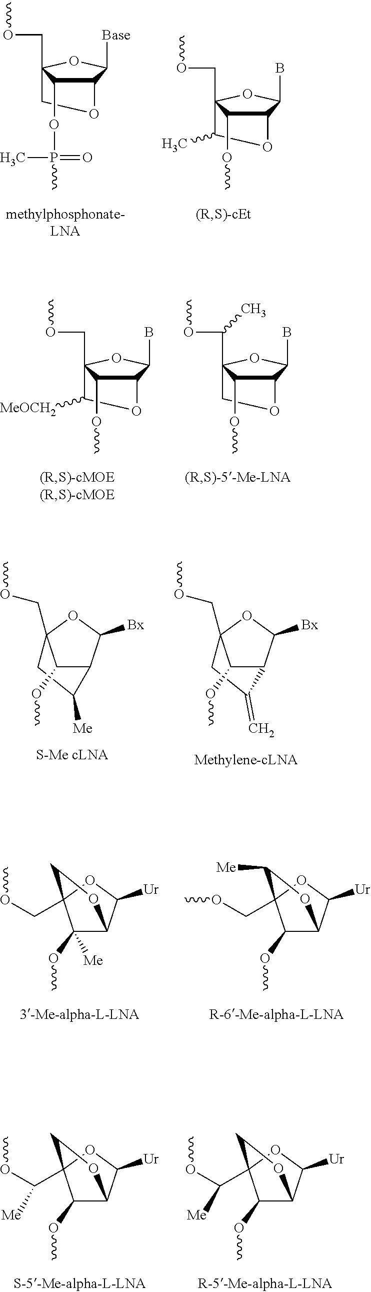

In some embodiments, R¹ is R as defined and described. In some embodiments, R² is R. In some embodiments, R^{c} is R. In some embodiments, Re is H, CH₃, Bn, COCF₃, benzoyl, benzyl, pyren-1-ylcarbonyl, pyren-1-ylmethyl, 2-aminoethyl. In some embodiments, an example modified internucleotidic linkage and/or sugar is selected from those described in Ts'o et al. Ann. N. Y. Acad. Sci. 1988, 507, 220; Gryaznov, S.; Chen, J.-K. J. Am. Chem. Soc. 1994, 116, 3143; Mesmaeker et al. Angew. Chem., Int. Ed. Engl. 9, 33, 226; Jones et al. J. Org. Chem. 9, 58, 2983; Vasseur et al. J. Am. Chem. Soc. 1992, 114, 4006; Van Aerschot et al. 1995 Angew. Chem. Int. Ed. Engl. 34: 1338; Hendrix et al. 1997 Chem. Eur. J. 3: 110; Koshkin et al. 1998 Tetrahedron 54: 3607-3630; Hyrup et al. 1996 Bioorg. Med. Chem. 4: 5; Nielsen et al. 1997 Chem. Soc. Rev. 73; Schultz et al. 1996 Nucleic Acids Res. 24: 2966; Obika et al. 1997 Tetrahedron Lett. 38 (50): 8735-8; Obika et al. 1998 Tetrahedron Lett. 39: 5401-5404; Singh et al. 1998 Chem. Comm. 1247-1248; Kumar et al. 1998 Bioo. Med. Chem. Let. 8: 2219-2222; Nielsen et al. 1997 J. Chem. Soc. Perkins Transl. 1: 3423-3433; Singh et al. 1998 J. Org. Chem. 63: 6078-6079; Seth et al. 2010 J. Org. Chem. 75: 1569-1581; Singh et al. 1998 J. Org. Chem. 63: 10035-39; Sorensen 2003 Chem. Comm. 2130-2131; Petersen et al. 2003 TRENDS Biotech. 21: 74-81; Rajwanshi et al. 1999 Chem. Commun. 1395-1396; Jepsen et al. 2004 Oligo. 14: 130-146; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Koizumi et al. 2003 Nuc. Acids Res. 12: 3267-3273; Lauritsen et al. 2002 Chem. Comm. 5: 530-531; Lauritsen et al. 2003 Bioo. Med. Chem. Lett. 13: 253-256; WO 20070900071; Seth et al., Nucleic Acids Symposium Series (2008), 52(1), 553-554; Seth et al. 2009 J. Med. Chem. 52: 10-13; Seth et al. 2012 Mol. Ther-Nuc. Acids. 1, e47; Pallan et al. 2012 Chem. Comm. 48: 8195-8197; Seth et al. 2010 J. Med. Chem. 53: 8309-8318; Seth et al. 2012 Bioo. Med. Chem. Lett. 22: 296-299; WO 2016/079181; U.S. Pat. Nos. 6,326,199; 6,066,500; and 6,440,739, the base and sugar modifications of each of which is herein incorporated by reference.

In some embodiments, the present disclosure provides oligonucleotides and oligonucleotide compositions that are chirally controlled. For instance, in some embodiments, a provided composition contains predetermined levels of one or more individual oligonucleotide types, wherein an oligonucleotide type is defined by: 1) base sequence; 2) pattern of backbone linkages; 3) pattern of backbone chiral centers; and 4) pattern of backbone P-modifications. In some embodiments, a particular oligonucleotide type may be defined by 1A) base identity; 1B) pattern of base modification; 1C) pattern of sugar modification; 2) pattern of backbone linkages; 3) pattern of backbone chiral centers; and 4) pattern of backbone P-modifications. In some embodiments, oligonucleotides of the same oligonucleotide type are identical.

In some embodiments, a provided oligonucleotide is a unimer. In some embodiments, a provided oligonucleotide is a P-modification unimer. In some embodiments, a provided oligonucleotide is a stereounimer. In some embodiments, a provided oligonucleotide is a stereounimer of configuration Rp. In some embodiments, a provided oligonucleotide is a stereounimer of configuration Sp.

In some embodiments, a provided oligonucleotide is an altmer. In some embodiments, a provided oligonucleotide is a P-modification altmer. In some embodiments, a provided oligonucleotide is a stereoaltmer.

In some embodiments, a provided oligonucleotide is a blockmer. In some embodiments, a provided oligonucleotide is a P-modification blockmer. In some embodiments, a provided oligonucleotide is a stereoblockmer.

In some embodiments, a provided oligonucleotide is a gapmer.

In some embodiments, a provided oligonucleotide is a skipmer.

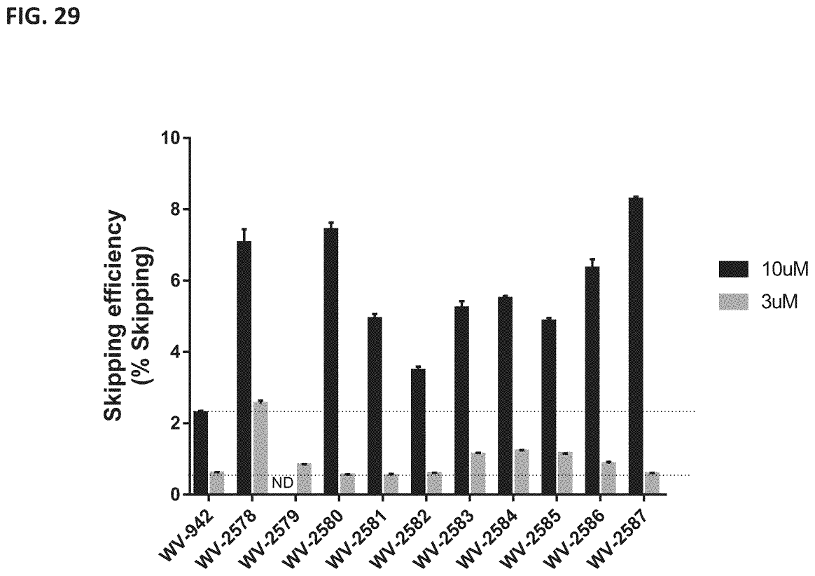

In some embodiments, a provided oligonucleotide is a hemimer. In some embodiments, a hemimer is an oligonucleotide wherein the 5′-end or the 3′-end has a sequence that possesses a structure feature that the rest of the oligonucleotide does not have. In some embodiments, the 5′-end or the 3′-end has or comprises 2 to 20 nucleotides. In some embodiments, a structural feature is a base modification. In some embodiments, a structural feature is a sugar modification. In some embodiments, a structural feature is a P-modification. In some embodiments, a structural feature is stereochemistry of the chiral internucleotidic linkage. In some embodiments, a structural feature is or comprises a base modification, a sugar modification, a P-modification, or stereochemistry of the chiral internucleotidic linkage, or combinations thereof. In some embodiments, a hemimer is an oligonucleotide in which each sugar moiety of the 5′-end sequence shares a common modification. In some embodiments, a hemimer is an oligonucleotide in which each sugar moiety of the 3′-end sequence shares a common modification. In some embodiments, a common sugar modification of the 5′ or 3′ end sequence is not shared by any other sugar moieties in the oligonucleotide. In some embodiments, an example hemimer is an oligonucleotide comprising a sequence of substituted or unsubstituted 2′-O-alkyl sugar modified nucleosides, bicyclic sugar modified nucleosides, p-D-ribonucleosides or p-D-deoxyribonucleosides (for example 2′-MOE modified nucleosides, and LNA™ or ENA™ bicyclic sugar modified nucleosides) at one terminus and a sequence of nucleosides with a different sugar moiety (such as a substituted or unsubstituted 2′-O-alkyl sugar modified nucleosides, bicyclic sugar modified nucleosides or natural ones) at the other terminus. In some embodiments, a provided oligonucleotide is a combination of one or more of unimer, altmer, blockmer, gapmer, hemimer and skipmer. In some embodiments, a provided oligonucleotide is a combination of one or more of unimer, altmer, blockmer, gapmer, and skipmer. For instance, in some embodiments, a provided oligonucleotide is both an altmer and a gapmer. In some embodiments, a provided nucleotide is both a gapmer and a skipmer. One of skill in the chemical and synthetic arts will recognize that numerous other combinations of patterns are available and are limited only by the commercial availability and/or synthetic accessibility of constituent parts required to synthesize a provided oligonucleotide in accordance with methods of the present disclosure. In some embodiments, a hemimer structure provides advantageous benefits, as exemplified by FIG. 29. In some embodiments, provided oligonucleotides are 5′-hemmimers that comprises modified sugar moieties in a 5′-end sequence. In some embodiments, provided oligonucleotides are 5′-hemmimers that comprises modified 2′-sugar moieties in a 5′-end sequence.

In some embodiments, a provided oligonucleotide comprises one or more optionally substituted nucleotides. In some embodiments, a provided oligonucleotide comprises one or more modified nucleotides. In some embodiments, a provided oligonucleotide comprises one or more optionally substituted nucleosides. In some embodiments, a provided oligonucleotide comprises one or more modified nucleosides. In some embodiments, a provided oligonucleotide comprises one or more optionally substituted LNAs.

In some embodiments, a provided oligonucleotide comprises one or more optionally substituted nucleobases. In some embodiments, a provided oligonucleotide comprises one or more optionally substituted natural nucleobases. In some embodiments, a provided oligonucleotide comprises one or more optionally substituted modified nucleobases. In some embodiments, a provided oligonucleotide comprises one or more 5-methylcytidine; 5-hydroxymethylcytidine, 5-formylcytosine, or 5-carboxylcytosine. In some embodiments, a provided oligonucleotide comprises one or more 5-methylcytidine.

In some embodiments, a provided oligonucleotide comprises one or more optionally substituted sugars. In some embodiments, a provided oligonucleotide comprises one or more optionally substituted sugars found in naturally occurring DNA and RNA. In some embodiments, a provided oligonucleotide comprises one or more optionally substituted ribose or deoxyribose. In some embodiments, a provided oligonucleotide comprises one or more optionally substituted ribose or deoxyribose, wherein one or more hydroxyl groups of the ribose or deoxyribose moiety is optionally and independently replaced by halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen. In some embodiments, a provided oligonucleotide comprises one or more optionally substituted deoxyribose, wherein the 2′ position of the deoxyribose is optionally and independently substituted with one or more —F. halogen. In some embodiments, a provided oligonucleotide comprises one or more optionally substituted deoxyribose, wherein the 2′ position of the deoxyribose is optionally and independently substituted with —OR′, wherein each R′ is independently as defined above and described herein. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ aliphatic. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ alkyl. In some embodiments, a provided oligonucleotide comprises one or more optionally substituted deoxyribose, wherein the 2′ position of the deoxyribose is optionally and independently substituted with —OMe. In some embodiments, a provided oligonucleotide comprises one or more optionally substituted deoxyribose, wherein the 2′ position of the deoxyribose is optionally and independently substituted with —O-methoxyethyl.

In some embodiments, a provided oligonucleotide is single-stranded oligonucleotide.

In some embodiments, a provided oligonucleotide is a hybridized oligonucleotide strand. In certain embodiments, a provided oligonucleotide is a partially hydridized oligonucleotide strand. In certain embodiments, a provided oligonucleotide is a completely hydridized oligonucleotide strand. In certain embodiments, a provided oligonucleotide is a double-stranded oligonucleotide. In certain embodiments, a provided oligonucleotide is a triple-stranded oligonucleotide (e.g., a triplex).

In some embodiments, a provided oligonucleotide is chimeric. For example, in some embodiments, a provided oligonucleotide is DNA-RNA chimera, DNA-LNA chimera, etc.

In some embodiments, any one of the structures comprising an oligonucleotide depicted in WO2012/030683 can be modified in accordance with methods of the present disclosure to provide chirally controlled variants thereof. For example, in some embodiments the chirally controlled variants comprise a stereochemical modification at any one or more of the linkage phosphorus and/or a P-modification at any one or more of the linkage phosphorus. For example, in some embodiments, a particular nucleotide unit of an oligonucleotide of WO2012/030683 is preselected to be stereochemically modified at the linkage phosphorus of that nucleotide unit and/or P-modified at the linkage phosphorus of that nucleotide unit. e.g., The related disclosure of WO2012/030683 is herein incorporated by reference in its entirety.

In some embodiments, a provided oligonucleotide is a therapeutic agent.

In some embodiments, a provided oligonucleotide is an antisense oligonucleotide.

In some embodiments, a provided oligonucleotide is an antigene oligonucleotide.

In some embodiments, a provided oligonucleotide is a decoy oligonucleotide.

In some embodiments, a provided oligonucleotide is part of a DNA vaccine.

In some embodiments, a provided oligonucleotide is an immunomodulatory oligonucleotide, e.g., immunostimulatory oligonucleotide and immunoinhibitory oligonucleotide.

In some embodiments, a provided oligonucleotide is an adjuvant.

In some embodiments, a provided oligonucleotide is an aptamer.

In some embodiments, a provided oligonucleotide is a ribozyme.

In some embodiments, a provided oligonucleotide is a deoxyribozyme (DNAzymes or DNA enzymes).

In some embodiments, a provided oligonucleotide is an siRNA.

In some embodiments, a provided oligonucleotide is a microRNA, or miRNA.

In some embodiments, a provided oligonucleotide is a ncRNA (non-coding RNAs), including a long non-coding RNA (lncRNA) and a small non-coding RNA, such as piwi-interacting RNA (piRNA).

In some embodiments, a provided oligonucleotide is complementary to a structural RNA, e.g., tRNA.

In some embodiments, a provided oligonucleotide is a nucleic acid analog, e.g., GNA, LNA, PNA, TNA and Morpholino.

In some embodiments, a provided oligonucleotide is a P-modified prodrug.

In some embodiments, a provided oligonucleotide is a primer. In some embodiments, a primers is for use in polymerase-based chain reactions (i.e., PCR) to amplify nucleic acids. In some embodiments, a primer is for use in any known variations of PCR, such as reverse transcription PCR (RT-PCR) and real-time PCR.

In some embodiments, a provided oligonucleotide is characterized as having the ability to modulate RNase H activation. For example, in some embodiments, RNase H activation is modulated by the presence of stereocontrolled phosphorothioate nucleic acid analogs, with natural DNA/RNA being more or equally susceptible than the Rp stereoisomer, which in turn is more susceptible than the corresponding Sp stereoisomer.

In some embodiments, a provided oligonucleotide is characterized as having the ability to indirectly or directly increase or decrease activity of a protein or inhibition or promotion of the expression of a protein. In some embodiments, a provided oligonucleotide is characterized in that it is useful in the control of cell proliferation, viral replication, and/or any other cell signaling process.

In some embodiments, a provided oligonucleotide is from about 2 to about 200 nucleotide units in length. In some embodiments, a provided oligonucleotide is from about 2 to about 180 nucleotide units in length. In some embodiments, a provided oligonucleotide is from about 2 to about 160 nucleotide units in length. In some embodiments, a provided oligonucleotide is from about 2 to about 140 nucleotide units in length. In some embodiments, a provided oligonucleotide is from about 2 to about 120 nucleotide units in length. In some embodiments, a provided oligonucleotide is from about 2 to about 100 nucleotide units in length. In some embodiments, a provided oligonucleotide is from about 2 to about 90 nucleotide units in length. In some embodiments, a provided oligonucleotide is from about 2 to about 80 nucleotide units in length. In some embodiments, a provided oligonucleotide is from about 2 to about 70 nucleotide units in length. In some embodiments, a provided oligonucleotide is from about 2 to about 60 nucleotide units in length. In some embodiments, a provided oligonucleotide is from about 2 to about 50 nucleotide units in length. In some embodiments, a provided oligonucleotide is from about 2 to about 40 nucleotide units in length. In some embodiments, a provided oligonucleotide is from about 2 to about 30 nucleotide units in length. In some embodiments, a provided oligonucleotide is from about 2 to about 29 nucleotide units in length. In some embodiments, a provided oligonucleotide is from about 2 to about 28 nucleotide units in length. In some embodiments, a provided oligonucleotide is from about 2 to about 27 nucleotide units in length. In some embodiments, a provided oligonucleotide is from about 2 to about 26 nucleotide units in length. In some embodiments, a provided oligonucleotide is from about 2 to about 25 nucleotide units in length. In some embodiments, a provided oligonucleotide is from about 2 to about 24 nucleotide units in length. In some embodiments, a provided oligonucleotide is from about 2 to about 23 nucleotide units in length. In some embodiments, a provided oligonucleotide is from about 2 to about 22 nucleotide units in length. In some embodiments, a provided oligonucleotide is from about 2 to about 21 nucleotide units in length. In some embodiments, a provided oligonucleotide is from about 2 to about 20 nucleotide units in length.

In some embodiments, a provided oligonucleotide is from about 4 to about 200 nucleotide units in length. In some embodiments, a provided oligonucleotide is from about 4 to about 180 nucleotide units in length. In some embodiments, a provided oligonucleotide is from about 4 to about 160 nucleotide units in length. In some embodiments, a provided oligonucleotide is from about 4 to about 140 nucleotide units in length. In some embodiments, a provided oligonucleotide is from about 4 to about 120 nucleotide units in length. In some embodiments, a provided oligonucleotide is from about 4 to about 100 nucleotide units in length. In some embodiments, a provided oligonucleotide is from about 4 to about 90 nucleotide units in length. In some embodiments, a provided oligonucleotide is from about 4 to about 80 nucleotide units in length. In some embodiments, a provided oligonucleotide is from about 4 to about 70 nucleotide units in length. In some embodiments, a provided oligonucleotide is from about 4 to about 60 nucleotide units in length. In some embodiments, a provided oligonucleotide is from about 4 to about 50 nucleotide units in length. In some embodiments, a provided oligonucleotide is from about 4 to about 40 nucleotide units in length. In some embodiments, a provided oligonucleotide is from about 4 to about 30 nucleotide units in length. In some embodiments, a provided oligonucleotide is from about 4 to about 29 nucleotide units in length. In some embodiments, a provided oligonucleotide is from about 4 to about 28 nucleotide units in length. In some embodiments, a provided oligonucleotide is from about 4 to about 27 nucleotide units in length. In some embodiments, a provided oligonucleotide is from about 4 to about 26 nucleotide units in length. In some embodiments, a provided oligonucleotide is from about 4 to about 25 nucleotide units in length. In some embodiments, a provided oligonucleotide is from about 4 to about 24 nucleotide units in length. In some embodiments, a provided oligonucleotide is from about 4 to about 23 nucleotide units in length. In some embodiments, a provided oligonucleotide is from about 4 to about 22 nucleotide units in length. In some embodiments, a provided oligonucleotide is from about 4 to about 21 nucleotide units in length. In some embodiments, a provided oligonucleotide is from about 4 to about 20 nucleotide units in length.

In some embodiments, a provided oligonucleotide is from about 5 to about 10 nucleotide units in length. In some embodiments, a provided oligonucleotide is from about 10 to about 30 nucleotide units in length. In some embodiments, a provided oligonucleotide is from about 15 to about 25 nucleotide units in length. In some embodiments, a provided oligonucleotide is from about 5, 6, 7, 8, 9, 10, 11, 12, 13, 14, 15, 16, 17, 18, 19, 20, 21, 22, 23, 24, or 25 nucleotide units in length.

In some embodiments, an oligonucleotide is at least 2 nucleotide units in length. In some embodiments, an oligonucleotide is at least 3 nucleotide units in length. In some embodiments, an oligonucleotide is at least 4 nucleotide units in length. In some embodiments, an oligonucleotide is at least 5 nucleotide units in length. In some embodiments, an oligonucleotide is at least 6 nucleotide units in length. In some embodiments, an oligonucleotide is at least 7 nucleotide units in length. In some embodiments, an oligonucleotide is at least 8 nucleotide units in length. In some embodiments, an oligonucleotide is at least 9 nucleotide units in length. In some embodiments, an oligonucleotide is at least 10 nucleotide units in length. In some embodiments, an oligonucleotide is at least 11 nucleotide units in length. In some embodiments, an oligonucleotide is at least 12 nucleotide units in length. In some embodiments, an oligonucleotide is at least 13 nucleotide units in length. In some embodiments, an oligonucleotide is at least 14 nucleotide units in length. In some embodiments, an oligonucleotide is at least 15 nucleotide units in length. In some embodiments, an oligonucleotide is at least 16 nucleotide units in length. In some embodiments, an oligonucleotide is at least 17 nucleotide units in length. In some embodiments, an oligonucleotide is at least 18 nucleotide units in length. In some embodiments, an oligonucleotide is at least 19 nucleotide units in length. In some embodiments, an oligonucleotide is at least 20 nucleotide units in length. In some embodiments, an oligonucleotide is at least 21 nucleotide units in length. In some embodiments, an oligonucleotide is at least 22 nucleotide units in length. In some embodiments, an oligonucleotide is at least 23 nucleotide units in length. In some embodiments, an oligonucleotide is at least 24 nucleotide units in length. In some embodiments, an oligonucleotide is at least 25 nucleotide units in length. In some other embodiments, an oligonucleotide is at least 30 nucleotide units in length. In some other embodiments, an oligonucleotide is a duplex of complementary strands of at least 18 nucleotide units in length. In some other embodiments, an oligonucleotide is a duplex of complementary strands of at least 21 nucleotide units in length.

In some embodiments, the 5′-end and/or the 3′-end of a provided oligonucleotide is modified. In some embodiments, the 5′-end and/or the 3′-end of a provided oligonucleotide is modified with a terminal cap moiety. Example such modifications, including terminal cap moieties are extensively described herein and in the art, for example but not limited to those described in US Patent Application Publication US 2009/0023675A1.

In some embodiments, oligonucleotides of an oligonucleotide type characterized by 1) a common base sequence and length, 2) a common pattern of backbone linkages, and 3) a common pattern of backbone chiral centers, have the same chemical structure. For example, they have the same base sequence, the same pattern of nucleoside modifications, the same pattern of backbone linkages (i.e., pattern of internucleotidic linkage types, for example, phosphate, phosphorothioate, etc), the same pattern of backbone chiral centers (i.e. pattern of linkage phosphorus stereochemistry (Rp/Sp)), and the same pattern of backbone phosphorus modifications (e.g., pattern of “—XLR¹” groups in formula I).

The present disclosure provides compositions comprising or consisting of a plurality of provided oligonucleotides (e.g., chirally controlled oligonucleotide compositions). In some embodiments, all such provided oligonucleotides are of the same type, i.e., all have the same base sequence, pattern of backbone linkages (i.e., pattern of internucleotidic linkage types, for example, phosphate, phosphorothioate, etc), pattern of backbone chiral centers (i.e. pattern of linkage phosphorus stereochemistry (Rp/Sp)), and pattern of backbone phosphorus modifications (e.g., pattern of “—XLR¹” groups in formula I). In some embodiments, all oligonucleotides of the same type are identical. In many embodiments, however, provided compositions comprise a plurality of oligonucleotides types, typically in pre-determined relative amounts.

In some embodiments, a provided chirally controlled oligonucleotide composition comprises a combination of one or more provided oligonucleotide types. One of skill in the chemical and medicinal arts will recognize that the selection and amount of each of the one or more types of provided oligonucleotides in a provided composition will depend on the intended use of that composition. That is to say, one of skill in the relevant arts would design a provided chirally controlled oligonucleotide composition such that the amounts and types of provided oligonucleotides contained therein cause the composition as a whole to have certain desirable characteristics (e.g., biologically desirable, therapeutically desirable, etc.).

In some embodiments, a provided chirally controlled oligonucleotide composition comprises a combination of two or more provided oligonucleotide types. In some embodiments, a provided chirally controlled oligonucleotide composition comprises a combination of three or more provided oligonucleotide types. In some embodiments, a provided chirally controlled oligonucleotide composition comprises a combination of four or more provided oligonucleotide types. In some embodiments, a provided chirally controlled oligonucleotide composition comprises a combination of five or more provided oligonucleotide types. In some embodiments, a provided chirally controlled oligonucleotide composition comprises a combination of six or more provided oligonucleotide types. In some embodiments, a provided chirally controlled oligonucleotide composition comprises a combination of seven or more provided oligonucleotide types. In some embodiments, a provided chirally controlled oligonucleotide composition comprises a combination of eight or more provided oligonucleotide types. In some embodiments, a provided chirally controlled oligonucleotide composition comprises a combination of nine or more provided oligonucleotide types. In some embodiments, a provided chirally controlled oligonucleotide composition comprises a combination of ten or more provided oligonucleotide types. In some embodiments, a provided chirally controlled oligonucleotide composition comprises a combination of fifteen or more provided oligonucleotide types.

In some embodiments, a provided chirally controlled oligonucleotide composition is a combination of an amount of chirally uniform mipomersen of the Rp configuration and an amount of chirally uniform mipomersen of the Sp configuration.

In some embodiments, a provided chirally controlled oligonucleotide composition is a combination of an amount of chirally uniform mipomersen of the Rp configuration, an amount of chirally uniform mipomersen of the Sp configuration, and an amount of one or more chirally pure mipomersen of a desired diastereomeric form.

In some embodiments, a provided oligonucleotide type is selected from those described in WO/2014/012081 and WO/2015/107425, the oligonucleotides, oligonucleotide types, oligonucleotide compositions, and methods thereof of each of which are incorporated herein by reference. In some embodiments, a provided chirally controlled oligonucleotide composition comprises oligonucleotides of an oligonucleotide type selected from those described in WO/2014/012081 and WO/2015/107425.

### Incorporation of Lipids

Lipids can be incorporated into provided technologies through many types of methods in accordance with the present disclosure. In some embodiments, lipids are physically mixed with provided oligonucleotides to form provided compositions. In some embodiments, lipids are chemically conjugated with oligonucleotides.

In some embodiments, provided compositions comprise two or more lipids. In some embodiments, provided oligonucleotides comprise two or more conjugated lipids. In some embodiments, the two or more conjugated lipids are the same. In some embodiments, the two or more conjugated lipids are different. In some embodiments, provided oligonucleotides comprise no more than one lipid. In some embodiments, oligonucleotides of a provided composition comprise different types of conjugated lipids. In some embodiments, oligonucleotides of a provided composition comprise the same type of lipids.

Lipids can be conjugated to biologically active agents, e.g., oligonucleotides optionally through linkers. Various types of linkers in the art can be utilized in accordance of the present disclosure. In some embodiments, a linker comprise a phosphate group, which can, for example, be used for conjugating lipids through chemistry similar to those employed in oligonucleotide synthesis. In some embodiments, a linker comprises an amide, ester, or ether group. In some embodiments, a linker has the structure of -L^{LD}-. In some embodiments, a linker has the structure of -L-. In some embodiments, after conjugation to oligonucleotides, a lipid forms a moiety having the structure of -L^{LD}-R^{LD}, wherein each of L^{LD} and R^{LD} is independently as defined and described herein. In some embodiments, after conjugation to oligonucleotides, a lipid forms a moiety having the structure of -L-R^{LD}, wherein each of L and R^{LD} is independently as defined and described herein.

In some embodiments, -L- comprises a bivalent aliphatic chain. In some embodiments, -L- comprises a phosphate group. In some embodiments, -L- comprises a phosphorothioate group. In some embodiments, -L- has the structure of —C(O)NH—(CH₂)₆—OP(═O)(S⁻)—.

Lipids, optionally through linkers, can be conjugated to oligonucleotides at various suitable locations. In some embodiments, lipids are conjugated through the 5′-OH group. In some embodiments, lipids are conjugated through the 3′-OH group. In some embodiments, lipids are conjugated through one or more sugar moieties. In some embodiments, lipids are conjugated through one or more bases. In some embodiments, lipids are incorporated through one or more internucleotidic linkages. In some embodiments, an oligonucleotide may contain multiple conjugated lipids which are independently conjugated through its 5′-OH, 3′-OH, sugar moieties, base moieties and/or internucleotidic linkages.

As demonstrated in the present disclosure, conjugations of lipids with oligonucleotides can surprising improve properties of the oligonucleotides, such as safety, activity, delivery, etc.

### Certain Biological Applications and Use

As described herein, provided compositions and methods are capable of altering splicing of transcripts. In some embodiments, provided compositions and methods provide improved splicing patterns of transcripts compared to a reference pattern, which is a pattern from a reference condition selected from the group consisting of absence of the composition, presence of a reference composition, and combinations thereof. An improvement can be an improvement of any desired biological functions. In some embodiments, for example, in DMD, an improvement is production of an mRNA from which a dystrophin protein with improved biological activities is produced. In some other embodiments, for example, an improvement is down-regulation of STAT3, HNRNPH1 and/or KDR to mitigate tumor progression, malignancy, and angiogenesis through forced splicing-induced nonsense-mediated decay (DSD-NMD).

In some embodiments, the present disclosure provides a method for altering splicing of a target transcript, comprising administering a composition comprising a first plurality of oligonucleotides, wherein the splicing of the target transcript is altered relative to reference conditions selected from the group consisting of absence of the composition, presence of a reference composition, and combinations thereof.

In some embodiments, the present disclosure provides a method of generating a set of spliced products from a target transcript, the method comprising steps of: contacting a splicing system containing the target transcript with a provided composition, in an amount, for a time, and under conditions sufficient for a set of spliced products to be generated that is different from a set generated under reference conditions selected from the group consisting of absence of the lipids in the provided composition, the composition, presence of a reference composition, and combinations thereof.

As widely known in the art, many diseases and/or conditions are associated with transcript splicing. For examples, see Garcia-Blanco, et al., Alternative splicing in disease and therapy, Nat Biotechnol. 2004 May; 22(5):535-46; Wang, et al., Splicing in disease: disruption of the splicing code and the decoding machinery, Nat Rev Genet. 2007 October; 8(10):749-61; Havens, et al., Targeting RNA splicing for disease therapy, Wiley Interdiscip Rev RNA. 2013 May-June; 4(3):247-66; Perez, et al., Antisense mediated splicing modulation for inherited metabolic diseases: challenges for delivery, Nucleic Acid Ther. 2014 February; 24(1):48-56; etc. Additional example targets and/or disease are described in Xiong, et al., The human splicing code reveals new insights into the genetic determinants of disease, Science. 2015 Jan. 9; 347(6218):1254806. doi: 10.1126/science.1254806. In some embodiments, the present disclosure provides compositions and methods for treating or preventing diseases, including but not limited to those described in references cited in this disclosure.

In some embodiments, the present disclosure provides a method for treating or preventing a disease, comprising administering to a subject an oligonucleotide composition described herein.

In some embodiments, the present disclosure provides a method for treating or preventing a disease, comprising administering to a subject a provided oligonucleotide composition comprising a lipid and a first plurality of oligonucleotides to which the lipid is conjugated, which oligonucleotides:
1) have a common base sequence complementary to a target sequence in a transcript; and
2) comprise one or more modified sugar moieties and modified internucleotidic linkages,

the oligonucleotide composition being characterized in that, when it is contacted with the transcript in a transcript splicing system, splicing of the transcript is altered relative to that observed under reference conditions selected from the group consisting of absence of the lipid, absence of the composition, presence of a reference composition, and combinations thereof.

In some embodiments, the present disclosure provides a method for treating or preventing a disease, comprising administering to a subject an oligonucleotide composition comprising a lipid and a first plurality of oligonucleotides of a particular oligonucleotide type defined by:
1) base sequence;
2) pattern of backbone linkages;
3) pattern of backbone chiral centers; and
4) pattern of backbone phosphorus modifications,
   which composition is chirally controlled in that it is enriched, relative to a substantially racemic preparation of oligonucleotides having the same base sequence, for oligonucleotides of the particular oligonucleotide type, wherein:

the lipid is conjugated to one or more oligonucleotides of the first plurality; and

the oligonucleotide composition being characterized in that, when it is contacted with the transcript in a transcript splicing system, splicing of the transcript is altered relative to that observed under reference conditions selected from the group consisting of absence of the composition, presence of a reference composition, and combinations thereof.

In some embodiments, a disease is one in which, after administering a provided composition, one or more spliced transcripts repair, restore or introduce a new beneficial function. For example, in DMD, after skipping one or more exons, functions of dystrophin can be restored, or partially restored, through a truncated but (partially) active version. Other examples include but are not limited to those listed in Table ES1, ES2, or ES3. In some embodiments, a target is one listed in Table ES3 with “Correction of Aberrant Splicing”.

In some embodiments, a disease is one in which, after administering a provided composition, one or more spliced transcripts repair, a gene is effectively knockdown by altering splicing of the gene transcript. Examples include but are not limited to those listed in Table ES1, ES2, or ES3. In some embodiments, a target is one listed in Table ES3 with “Knockdown of Detrimental Gene Expression”.

In some embodiments, a disease is Duchenne muscular dystrophy. In some embodiments, a disease is spinal muscular atrophy. In some embodiments, a disease is cancer.

In some embodiments, the present disclosure provides a method of treating a disease by administering a composition comprising a first plurality of oligonucleotides sharing a common base sequence comprising a common base sequence, which nucleotide sequence is complementary to a target sequence in the target transcript,

the improvement that comprises using as the oligonucleotide composition a stereocontrolled oligonucleotide composition characterized in that 1) a lipid is conjugated to one or more oligonucleotides of the stereocontrolled oligonucleotide composition; and 2) when it is contacted with the transcript in a transcript splicing system, splicing of the transcript is altered relative to that observed under reference conditions selected from the group consisting of absence of the composition, presence of a reference composition, and combinations thereof.

In some embodiments, the present disclosure provides a method of treating a disease by administering a composition comprising a first plurality of oligonucleotides sharing a common base sequence comprising a common base sequence, which nucleotide sequence is complementary to a target sequence in the target transcript,

the improvement that comprises using as the oligonucleotide composition a stereocontrolled oligonucleotide composition characterized in that, 1) a lipid is conjugated to one or more oligonucleotides of the stereocontrolled oligonucleotide composition; and 2) when it is contacted with the transcript in a transcript splicing system, splicing of the transcript is altered relative to that observed under reference conditions selected from the group consisting of absence of the composition, presence of a reference composition, and combinations thereof.

In some embodiments, sequence of provide oligonucleotides is or comprises an element that is substantially complementary to a targeted element in a cellular nucleic acid. In some embodiments, a sequence is or comprises a sequence element that is associated with a muscle disease, disorder or condition. In some embodiments, a cellular nucleic acid is or comprises a transcript. In some embodiments, a cellular nucleic acid is or comprises a primary transcript. In some embodiments, a cellular nucleic acid is RNA. In some embodiments, a cellular nucleic acid is pre-mRNA. In some embodiments, a cellular nucleic acid is mRNA. In some embodiments, a cellular nucleic acid is or comprises genomic nucleic acid. In some embodiments, a sequence is or comprises an element that is substantially complementary to a targeted an RNA, and provided oligonucleotides of the sequence provide exon-skipping to form mRNA which are translated into proteins that have improved functions than proteins formed absence of the provided oligonucleotides. In some embodiments, such proteins with improved activities can restore or partially restore one or more muscular functions and can be used for treatment of muscle diseases, disorders and/or conditions.

In some embodiments, a common sequence of a plurality of oligonucleotides comprises a sequence selected from Table. In some embodiments, a common sequence is a sequence selected from Table ES1. In some embodiments, a common sequence is a sequence found is a transcript of any of the genes selected from Table ES1, ES2, and ES3.

Example diseases that can be treated include but are not limited to those described in Tables ES2 and ES3. In some embodiments, a disease is Duchenne muscular dystrophy. In some embodiments, a disease is spinal muscular atrophy. In some embodiments, a disease is cancer.

For Duchenne muscular dystrophy, example mutations and/or suitable DMD exons for skipping are widely known in the art, including but not limited to those described in U.S. Pat. Nos. 8,759,507, 8,486,907, and reference cited therein. In some embodiments, one or more skipped exons are selected from exon 2, 29, 40, 41, 42, 43, 44, 45, 46, 47, 48, 49, 50, 51 and 53. In some embodiments, exon 2 of DMD is skipped. In some embodiments, exon 29 of DMD is skipped. In some embodiments, exon 40 of DMD is skipped. In some embodiments, exon 41 of DMD is skipped. In some embodiments, exon 42 of DMD is skipped. In some embodiments, exon 43 of DMD is skipped. In some embodiments, exon 44 of DMD is skipped. In some embodiments, exon 45 of DMD is skipped. In some embodiments, exon 46 of DMD is skipped. In some embodiments, exon 47 of DMD is skipped. In some embodiments, exon 48 of DMD is skipped. In some embodiments, exon 49 of DMD is skipped. In some embodiments, exon 50 of DMD is skipped. In some embodiments, exon 51 of DMD is skipped. In some embodiments, exon 53 of DMD is skipped. In some embodiments, a skipped exon is any exon whose inclusion decreases a desired function of DMD. In some embodiments, a skipped exon is any exon whose skipping increased a desired function of DMD.

In some embodiments, for exon skipping of DMD transcript, or for treatment of DMD, a sequence of a provided plurality of oligonucleotides comprises a DMD sequence selected from Table ES1. In some embodiments, a sequence comprises one of SEQ ID Nos 1-30 of U.S. Pat. No. 8,759,507. In some embodiments, a sequence comprises one of SEQ ID Nos 1-211 of U.S. Pat. No. 8,486,907. In some embodiments, for exon skipping of DMD transcript, or for treatment of DMD, a sequence of a provided plurality of oligonucleotides is a DMD sequence selected from Table ES1. In some embodiments, a sequence is one of SEQ ID Nos 1-30 of U.S. Pat. No. 8,759,507. In some embodiments, a sequence is one of SEQ ID Nos 1-211 of U.S. Pat. No. 8,486,907. In some embodiments, a sequence comprises UCAAGGAAGAUGGCAUUUCU (SEQ ID NO: 1). In some embodiments, a sequence is UCAAGGAAGAUGGCAUUUCU (SEQ ID NO: 1). In some embodiments, a sequence comprises CTCCAACATCAAGGAAGATGGCATTTCTAG (SEQ ID NO: 9). In some embodiments, a sequence is CTCCAACATCAAGGAAGATGGCATTTCTAG (SEQ ID NO: 9).

In some embodiments, a sequence is selected from Table 4A. In some embodiments, a sequence is one described in Kemaladewi, et al., Dual exon skipping in myostatin and dystrophin for Duchenne muscular dystrophy, BMC Med Genomics. 2011 Apr. 20; 4:36. doi: 10.1186/1755-8794-4-36; or Malerba et al., Dual Myostatin and Dystrophin Exon Skipping by Morpholino Nucleic Acid Oligomers Conjugated to a Cell-penetrating Peptide Is a Promising Therapeutic Strategy for the Treatment of Duchenne Muscular Dystrophy, Mol Ther Nucleic Acids. 2012 Dec. 18; 1:e62. doi: 10.1038/mtna.2012.54.

In some embodiments, a disease treatment comprises knockdown of a gene function by altering its transcript splicing. Example disease and target genes include but are not limited to those listed in Table ES3, particularly those with labeled with “Knockdown of Detrimental Gene Expression”.

TABLE ES1
Example sequences (SEQ ID NOS 10-204,
respectively, in order of columns).

cccauuuugugaauguuuucuuuu
uuguguauuuacccauuuugug
Uauccucugaaugucgcauc
gguuauccucugaaugucgu
Gagccuuuuuucuucuuug
Uccuuucgucucugggcuc
Cuccucuuucuucuucugc
Cuucgaaacugagcaaauuu
cuugugagacaUgagug
cagagacuccucuugcuu
ugcugcugucuucuugcu
Uuguuaacuuuuucccauu
cgccgccauuucucaacag
TAGATAGCTATATAT
ATAGATAGCTATATA
TATAGATAGCTATAT
ATATAGATAGCTATA
GATATAGATAGCTAT
ATAGATAGCTAT
AGATATAGATAGCTA
TATAGATAGCTA
TAGATATAGATAGCT
ATATAGATAGCT
ATAGATATAGATAGC
GATATAGATAGC
TATAGATATAGATAG
AGATATAGATAG
ATATAGATATAGATA
TAGATATAGATA
TATATAGATATAGAT
ATAGATATAGAT
TATAGATATAGA
ATATAGATATAG
ATAGCTATATAGATA
AAAAAATAGCTATAT
GTTAAAAAAAATAGC
AGGAAGTTAAAAAAA
AATAAAGGAAGTTAA
AGGAAAATAAAGGAA
GTGTAAGGAAAATAA
ATTTTGTCTAAAACC
GATTTTGTCTAAAAC
TTTTGTCTAAAA
TGATTTTGTCTAAAA
ATTTTGTCTAAA
TTGATTTTGTCTAAA
GATTTTTGTCTAA
TTTGATTTTGTCTAA
TTTTGATTTTGTCTAA
TTTTGATTTTGTCTA
TGATTTTGTCTA
TTGATTTTGTCT
TTTTTGATTTTGTCT
TTTGATTTTGTC
CTTTTTGATTTTGTC
TTTTGATTTTGT
TTTTTGATTTTG
CTTCTTTTTGATTTT
CTTTTTGATTTT
TCTTTTTGATTT
CCTTCCTTCTTTTTG
GAGCACCTTCCTTCT
AATGTGAGCACCTTC
TAAGGAATGTGAGCA
AATTTAAGGAATGTGAGC
TTAAGGAATGTGAGC
TAATTTAAGGAATGTGAG
TTTAAGGAATGTGAG
AAGGAATGTGAG
TTAATTTAAGGAATGTGA
ATTTAAGGAATGTGA
TAAGGAATGTGA
CTTAATTTAAGGAATGTG
AATTTAAGGAATGTG
TTAAGGAATGTG
TAATTTAAGGAATGT
CCTTAATTTAAGGAATGT
TTTAAGGAATGT
TTAATTTAAGGAATG
ATTTAAGGAATG
CTTAATTTAAGGAAT
AATTTAAGGAAT
CCTTAATTTAAGGAA
TAATTTAAGGAA
TCCTTAATTTAAGGA
TTAATTTAAGGA
CTTAATTTAAGG
CCTTAATTTAAG
TGCTGGCAGACTTAC
CATAATGCTGGCAGA
TCATAATGCTGGCAG
TTCATAATGCTGGCA
TTTCATAATGCTGGC
ATTCACTTTCATAATGCTGG
CTTTCATAATGCTGG
TCATAATGCTGG
ACTTTCATAATGCTG
TTCATAATGCTG
CACTTTCATAATGCT
TTTCATAATGCT
TCACTTTCATAATGC
GTTTCATAATGC
TTCACTTTCATAATG
ACTTTCATAATG
ATTCACTTTCATAAT
CACTTTCATAAT
GATTCACTTTCATAA
TCACTTTCATAA
TTCACTTTCATA
ATTCACTTTCAT
AGTAAGATTCACTTT
ACAAAAGTAAGATTC
GTTTTACAAAAGTAA
ATAAAGTTTTACAAA
AAACCATAAAGTTTT
TCCACAAACCATAAA
ATT CAC TTT CAT AAT GCT GG
ATT CAC TTT CAT AAT GCT GG
ATT CAC TTT CAT AAT GCT GG
ATT CAC TTT CAT AAT GCT GG
ATT CAC TTT CAT AAT GCT GG
ATT CAC TTT CAT AAT GCT GG
ATT CAC TTT CAT AAT GCT GG
ATT CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
CAC TTT CAT AAT GCT GG
TTT CAT AAT GCT GG
TTT CAT AAT GCT GG
TTT CAT AAT GCT GG
TTT CAT AAT GCT GG
TTT CAT AAT GCT GG
TTT CAT AAT GCT GG
TTT CAT AAT GCT GG
AAT GCT GGC AG
AAT GCT GGC AG
AAT GCT GGC AG
AAT GCT GGC AG
AAT GCT GGC AG
AAT GCT GGC AG
AAT GCT GGC AG
GCT GGC AG
GCT GGC AG
GCT GGC AG
GCT GGC AG
GCT GGC AG
GCT GGC AG
GCT GGC AG
GCT GGC AG
GCT GGC AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C TAG TAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
C CAG CAT TTC CTG CAA ATG AG
A TGC CAG CAT TTC CTG CAA ATG AGA
A TGC CAG CAT TTC CTG CAA ATG AGA
A TGC CAG CAT TTC CTG CAA ATG AGA
A TGC CAG CAT TTC CTG CAA ATG AGA
A TGC CAG CAT TTC CTG CAA ATG AGA
A TGC CAG CAT TTC CTG CAA ATG AGA
A TGC CAG CAT TTC CTG CAA ATG AGA
A TGC CAG CAT TTC CTG CAA ATG AGA
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A
GCT CTA TGC CAG CAT TTC CTG CAA A

SEQ ID Nos 1-30 of U.S. Pat. No. 8,759,507;
SEQ ID Nos 1-211 of U.S. Pat. No. U.S. Pat. No. 8,486,907;

TABLE ES2

| | | | | | | | |
|---|---|---|---|---|---|---|---|
| | | | | | Repeat | | |
| | | | Parent of | Repeat | number | Repeat | |
| | | | origin of | number | (pre- | number | Somatic |
| Disease | Sequence | Location | expansion | (normal) | mutation) | (disease) | instability |

Diseases with coding TNRs

| | | | | | | | |
|---|---|---|---|---|---|---|---|
| DRPLA | CAG | ATN1 (exon 5) | P | 6-35 | 35-48 | 49-88 | Yes |
| HD | CAG | HTT (exon 1) | P | 6-29 | 29-37 | 38-180 | Yes |
| OPMD | GCN | PABPN1 | P and M | 10 | 12-17 | >11 | None found in |
| | | (exon 1) | | | | | tissue tested |
| | | | | | | | (hypothalamus) |
| SCA1 | CAG | ATXN1 | P | 6-39 | 40 | 41-83 | Yes |
| | | (exon 8) | | | | | |
| SCA2 | CAG | ATXN2 | P | <31 | 31-32 | 32-200 | Unknown |
| | | (exon 1) | | | | | |
| SCA3 | CAG | ATXN3 | P | 12-40 | 41-85 | 52-86 | Unknown |
| (Machado- | | (exon 8) | | | | | |
| Joseph | | | | | | | |
| disease) | | | | | | | |
| SCA6 | CAG | CACNA1A | P | <18 | 19 | 20-33 | None found |
| | | (exon 47) | | | | | |
| SCA7 | CAG | ATXN7 | P | 4-17 | 28-33 | >36 | Yes |
| | | (exon 3) | | | | to >460 | |
| SCA17 | CAG | TBP (exon 3) | P > M | 25-42 | 43-48 | 45-66 | Yes |
| SMBA | CAG | AR (exon 1) | P | 13-31 | 32-39 | 40 | None found |

Diseases with non-coding TNRs

| | | | | | | | |
|---|---|---|---|---|---|---|---|
| DM1 | CTG | DMPK (3′ UTR) | M | 5-37 | 37-50 | <50 | Yes |
| DM2 | CCTG | CNBP | Uncertain | <30 | 31-74 | 75-11,000 | Yes |
| | | (intron 1) | | | | | |
| FRAX-E | GCC | AFF2 (5′ UTR) | M | 4-39 | 40-200 | >200 | Unknown |
| FRDA | GAA | FXN (intron 1) | Recessive | 5-30 | 31-100 | 70-1,000 | Yes |
| FXS | CGG | FMR1 (5′ UTR) | M | 6-50 | 55-200 | 200-4,000 | Yes |
| HDL2 | CTG | JPH3 (exon 2A) | M | 6-27 | 29-35 | 36-57 | Unknown |
| SCA8 | CTG | ATXN8OS | M | 15-34 | 34-89 | 89-250 | Unknown |
| | | (3′ UTR) | | | | | |
| SCA10 | ATTCT | ATXN10 | M and P | 10-29 | 29-400 | 400-4,500 | Yes |
| | | (intron 9) | (smaller | | | | |
| | | | changes | | | | |
| | | | with M) | | | | |
| SCA12 | CAG | PPP2R2B | M and P | 7-28 | 28-66 | 66-78 | None found |
| | | (5′ UTR) | (more | | | | |
| | | | unstable | | | | |
| | | | with P) | | | | |

AFF2, AF4/FMR2 family, member 2;
AR, androgen receptor;
ATN1, atrophin 1;
ATXN, ataxin;
ATXN8OS, ATXN8 opposite strand (non-protein coding);
CACNA1A, calcium channel, voltage-dependent, P/Q type, alpha 1A subunit;
CNBP, CCHC-type zinc finger nucleic acid binding protein;
DM, myotonic dystrophy;
DMPK, dystrophia myotonica-protein kinase;
DRPLA, dentatorubral-pallidoluysian atrophy;
FMR1, fragile × mental retardation 1;
FRAX-E, mental retardation, X-linked, associated with FRAXE;
FRDA, Friedreich's ataxia;
FXN, frataxin;
FXS, fragile × syndrome;
FXTAS, fragile X-associated tremor/ataxia syndrome;
HD, Huntington's disease;
HDL2, Huntington's disease-like 2;
HTT, huntingtin;
JPH3, junctophilin 3;
M, maternal;
OPMD, oculopharyngeal muscular dystrophy;
P, paternal;
PABPN1, poly(A) binding protein nuclear 1;
PPP2R2B, protein phosphatase 2, regulatory subunit B;
SCA, spinocerebellar ataxia;
SMBA, spinomuscular bulbar atrophy;
TBP, TATA-box binding protein;
TNR, trinucleotide repeat.

| | | Ataxia telangiectasia | ATM | |
|---|---|---|---|---|
| | | β-Thalassemia | HBB | |
| | | Cancer | BRCA2 | |
| | | CDG1A² | PMM2 | |
| | | Congenital adrenal insufficiency | CYP11A | |
| | | Cystic fibrosis | CFTR | |
| Bardet-Biedl syndrome | BBS1 | Duchenne muscular dystrophy | DMD | |
| β-Thalassemia | HBB | Fukuyama congenital muscular | FKTN | |
| | | dystrophy (FCMD) | | |
| Cancer | BRCA1 | Growth hormone insensitivity | GHR | |
| | PTCH1 | HPABH4A² | PTS | |
| Cystic fibrosis | CFTR | Hutchinson-Gilford progeria (HGPS) | LMNA | |
| Duchenne muscular | DMD | MLC1² | MLC1 | |
| dystrophy | | | | |
| Factor VII deficiency | F7 | Methylmalonic aciduria | MUT | |
| Familial dysautonomia | IKBKAP | Myopathy with lactic acidosis | iSCU | |
| Fanconi anemia | FANCC | Myotonic dystrophy | CLC1 | |
| Hemophilia A | F9 | Neurofibromatosis | NF1 | |
| Propionic acidemia | PCCA | Niemann-Pick type C | NPC1 | |
| Retinitis pigmentosa | RHO | Propionic acidemia | PCCB | |
| | RPGR | Usher syndrome | USH1C | |
| | | Alzheimer's disease/FTDP-17 | MAPT | |
| | | Taupathies | | |
| | | Cancer | BCL2L1 | |
| | | | FGFR1 | |
| | | | MCL1 | |
| | | | MDM2 | |
| Afibrinogenemia | FGB | | Multiple | |
| Cancer | BRCA1 | | PKM | |
| Propionic acidemia | PCCA | | MST1R | |
| Neurofibromatosis | NF1 | | USP5 | |
| Ocular albinism type 1 | GRP143 | Spinal muscular atrophy | SMN2 | |
| Alzheimer's disease | BACE1 | | | |
| Cancer | CDKN1A | | | |
| | ERBB2 | | | |
| | FLT1 | | | |
| | HNRNPH1 | | | |
| | KDR | | | |
| | MYC | | | |
| | Multiple | | | |
| | PHB | | | |
| | SRA1 | | | |
| | STAT3 | | | |
| | TERT | | | |
| | WT1 | Duchenne muscular dystrophy | DMD | |
| FHBL/atherosclerosis² | APOB | | | |
| Immune-response | CD40 | | | |
| Inflammatory disease | TNFRSF1B | | | |
| | IL5RA | | | |
| Influenza virus | TMPRSS2 | Dystrophic epidermolysis bullosa | COL7A1 | |
| Muscle wasting diseases | MSTN | | | |
| Spinocerebellar ataxia | ATXN1 | Miyoshi myopathy | DYSF | |
| type 1 | | | | |
| Gene | Effect | Disease | Variant location | Effect on splicing/protein |

Modifies disease phenotype

| | | | | |
|---|---|---|---|---|
| CFTR | cis | Cystic fibrosis | (TG)n and Tn polymorphisms | Affects the amount of exon 9 skipping |
| | | | in CFTR intron 8 | |
| MCAD | cis | Medium-chain acyl-CoA | ESS within exon 5 | Prevents effect of disease-causing |
| | | dehydrogenase deficiency | | ESE mutation |
| SCN1A | cis | Susceptibility to anti- | 5′ splice site of neonatal | Increased use of neonatal alternative |
| | | epileptics | alternative exon | exon |
| CFTR | cis and | Cystic fibrosis | Point mutation in intron 19 creates a | Variable level of cryptic exon |
| | trans | | variably spliced 84-nucleotide exon | inclusion influences severity |
| IKBKAP | trans | Familial | n/a | Tissue-specific differences in |
| | | dysautonomia | | recognition of mutant 5′ splice site |
| Scn8a | cis and | Neurological disorder | 4-bp deletion within the 5′-splice | 5′ splice-site mutation modified by |
| | trans | (mouse) | site of exon 3 | Scnm1 |

Linked with disease susceptibility

| | | | | |
|---|---|---|---|---|
| IRF5 | cis | Systemic lupus | One SNP between alternative | SNP creates 5′ splice site and new |
| | | erythematosus (SLE) | promoters creates 5′ splice site | first exon |
| CTLA4 | cis | Autoimmune diseases | Two SNPs in 3′ UTR (exon 4) | Increased exon 3 skipping; reduced |
| | | | | soluble isoform |
| NCAM1 | cis | Bipolar disorder | Two SNPs, one within cluster | Decreased expression of secreted |
| | | | of alternative exons | splice variants |
| ERBB4 | cis | Schizophrenia | One SNP in intron 12 and | Increased use of exons 16 and 26 |
| | | | SNPs near exon 3 linked with | |
| | | | splicing of exons 16 and 26, | |
| | | | respectively | |
| OLR1 | cis | Myocardial infarction | Six SNPs; three in intron 4, | Exon 5 skipping results in an |
| | | | two in intron 5, one in | isoform with reduced |
| | | | the 3′ UTR in exon 6 | apoptotic effects |
| OAS1 | cis | Type 1 diabetes | Intron 6 AG→AA variant shifts | SNP moves splice site by 1 nucleotide |
| | | | 3′ splice site by 1 nucleotide, | resulting in a longer protein |
| | | | changing the reading frame | |
| TNNT2 | cis | Cardiac hypertrophy | 5-bp deletion affects intron | Results in E4 skipping (minigene |
| | | | 3 splice site | analysis) |
| GPRA | cis | Asthma | Three SNPs distal to alternative site | Increased use of the more distal of |
| | | | | two terminal exons |
| MAPT | cis | Tauopathies | 238-bp insertion into intron 9 | Enhanced exon 10 inclusion |
| PTPRC | cis | Altered immune | A138G polymorphism exon 6 | Enhanced exon 6 skipping |
| (CD45) | | function | | |
| PTPRC | cis | Multiple sclerosis | C77G polymorphism exon 4 | Enhanced exon 4 inclusion |
| (CD45) | | | | |
| LDLR | cis | Elevated cholesterol | C688T polymorphism exon 12 | Enhanced exon 12 skipping |
| SFRS8 | trans | Asthma | n/a | None reported |

| Splicing factorª | OMIM number^{b} | Disease association^{c} |
|---|---|---|
| CUG triplet repeat, RNA-binding protein 1; | 601074 | Myotonic dystrophy (DM) |
| CUGBP1 (CUGBP; NAB50; BRUNOL2) | | |
| CUG triplet repeat, RNA-binding protein 2; | 602538 | Myotonic dystrophy (DM) |
| CUGBP2 (ETR3) | | |
| FUS-interacting protein I; FUSIP1 | 605221 | Leukemias and sarcomas |
| (TASR(1 or 2); SRp38; SRRp40; NSSR) | | |
| Fusion, derived from 12-16 translocation, | 137070 | Liposarcomas, acute myeloid |
| malignant liposarcoma; FUS ( TLS) | | leukemia (AML) |
| Glycogen synthase kinase 3-BETA; GSK3B (GSK-3ß) | 605004 | Alzheimer disease (AD) |
| Hydroxymethylglutaryl coenzyme A1a | 600701 | Alzheimer disease (AD) |
| (HMGA1a) (HMG-I) | | |
| Muscleblind-like protein 1; MBNL1 (MBNL) | 606516 | Myotonic dystrophy (DM) |
| Muscleblind-like protein 2; MBNL2 (MBLL) | 607327 | Myotonic dystrophy (DM) |
| Muscleblind-like protein 3; MBNL3 (MBXL) | 300413 | Myotonic dystrophy (DM) |
| Neurooncologic ventral antigen 1; NOVA1 (Ri Ag) | 602157 | Paraneoplastic syndrome |
| Precursor mRNA-processing factor 3, | 607301 | Retinitis pigmentosa |
| Saccharomyces cerevisiae, homolog of | | |
| PRPF3 (PRP3; HPRP3) | | |
| Precursor mRNA-processing factor 31, | 606419 | Retinitis pigmentosa |
| S. cerevisiae, homolog of PRPF31 (PRP31) | | |
| Precursor mRNA-processing factor 8, | 607300 | Retinitis pigmentosa |
| S. cerevisiae, homolog of PRPF8 (PRP8 | | |
| PRPC8 U5 snRNP-specific protein, 220-K; p220) | | |
| RNA-binding motif protein, Y chromosome | 400006 | Azospermia |
| family 1, member A1; RBMY1A1 | | |
| (RBMY; RBM1; RBM2; YRRM1; YRRM2) | | |
| Splicing factor HCC1 (HCC1.3; HCC1.4) | 604739 | Hepatocellular carcinoma |
| Splicing factor, proline- and glutamine-rich SFPQ (PSF) | 605199 | Papillary renal cell carcinoma |
| Survival of motor neuron 1, telomeric: | 600354 | Spinal muscular atrophy |
| SMN1 (SMN; SMNT; T-BCD541) | | |
| Survival of motor neuron 2, centromeric, | 601627 | Spinal muscular atrophy |
| SMN2 (SMNC; C-BCD541) | | |
| Tumor protein p73-like: TP73L p(63) | 603273 | Hay-Wells syndrome |

| Disease | Human Target Gene | Gene Defects | Therapeutic modality | Approaches |
|---|---|---|---|---|
| Cancer | BRCA1 | Splice Site Mutations | ASO | Correction of Aberrant Splicing |
| Cancer | PTCH1 | Splice Site Mutations | ASO | Correction of Aberrant Splicing |
| Duchenne muscular | DMD | Splice Site Mutations | ASO | Correction of Aberrant Splicing |
| dystrophy | | | | |
| Ataxia telangiectasia | ATM | Cryptic Splice Sites | ASO | Correction of Aberrant Splicing |
| Beta-thalassemia | HBB | Cryptic Splice Sites | ASO | Correction of Aberrant Splicing |
| Cancer | BRCA2 | Cryptic Splice Sites | ASO | Correction of Aberrant Splicing |
| CDG1A | PMM2 | Cryptic Splice Sites | ASO | Correction of Aberrant Splicing |
| Congenital adrenal | CYP11A | Cryptic Splice Sites | ASO | Correction of Aberrant Splicing |
| insufficiency | | | | |
| Cystic fibrosis | CFTR | Cryptic Splice Sites | ASO | Correction of Aberrant Splicing |
| Duchenne muscular | DMD | Cryptic Splice Sites | ASO | Correction of Aberrant Splicing |
| dystrophy | | | | |
| Fukuyama congenital | FKTN | Cryptic Splice Sites | ASO | Correction of Aberrant Splicing |
| muscular dystrophy | | | | |
| (FCMD) | | | | |
| Growth hormone | GHR | Cryptic Splice Sites | ASO | Correction of Aberrant Splicing |
| insensitivity | | | | |
| HPABH4A | PTS | Cryptic Splice Sites | ASO | Correction of Aberrant Splicing |
| Hutchinson-Gilford | LMNA | Cryptic Splice Sites | ASO | Correction of Aberrant Splicing |
| progeria (HGPS) | | | | |
| MLC1 | MLC1 | Cryptic Splice Sites | ASO | Correction of Aberrant Splicing |
| Methylmalonic aciduria | MUT | Cryptic Splice Sites | ASO | Correction of Aberrant Splicing |
| Myopathy with lactic | ISCU | Cryptic Splice Sites | ASO | Correction of Aberrant Splicing |
| acidosis | | | | |
| Myotonic dystrophy | CLC1 | Cryptic Splice Sites | ASO | Correction of Aberrant Splicing |
| Neurofibromatosis | NF1 | Cryptic Splice Sites | ASO | Correction of Aberrant Splicing |
| Niemann-Pick type C | NPC1 | Cryptic Splice Sites | ASO | Correction of Aberrant Splicing |
| Propionic acidemia | PCCB | Cryptic Splice Sites | ASO | Correction of Aberrant Splicing |
| Usher syndrome | USH1C | Cryptic Splice Sites | ASO | Correction of Aberrant Splicing |
| Afibrinogenemia | FGB | Regulatory Sequence | ASO | Correction of Aberrant Splicing |
| | | Mutations | | |
| Cancer | BRCA1 | Regulatory Sequence | ASO | Correction of Aberrant Splicing |
| | | Mutations | | |
| Propionic acidemia | PCCA | Regulatory Sequence | ASO | Correction of Aberrant Splicing |
| | | Mutations | | |
| Ocular albinism type 1 | GRP143 | Regulatory Sequence | ASO | Correction of Aberrant Splicing |
| | | Mutations | | |
| Alzheimer's | MAPT | Deregulated Alternative | ASO | Correction of Aberrant Splicing |
| disease/FTDP-17 Taupathies | | Splicing | | |
| Cancer | BCL2L1 | Deregulated Alternative | ASO | Correction of Aberrant Splicing |
| | | Splicing | | |
| Cancer | FGFR1 | Deregulated Alternative | ASO | Correction of Aberrant Splicing |
| | | Splicing | | |
| Cancer | MCL1 | Deregulated Alternative | ASO | Correction of Aberrant Splicing |
| | | Splicing | | |
| Cancer | MDM2 | Deregulated Alternative | ASO | Correction of Aberrant Splicing |
| | | Splicing | | |
| Cancer | PKM | Deregulated Alternative | ASO | Correction of Aberrant Splicing |
| | | Splicing | | |
| Cancer | MST1R | Deregulated Alternative | ASO | Correction of Aberrant Splicing |
| | | Splicing | | |
| Cancer | USP5 | Deregulated Alternative | ASO | Correction of Aberrant Splicing |
| | | Splicing | | |
| Spinal muscular atrophy | SMN2 | Deregulated Alternative | ASO | Correction of Aberrant Splicing |
| | | Splicing | | |
| Alzheimer's disease | BACE1 | Detrimental | ASO | Knockdown of Detrimental |
| | | Gene Expression | | Gene Expression |
| Cancer | ERBB2 | Detrimental | ASO | Knockdown of Detrimental |
| | | Gene Expression | | Gene Expression |
| Cancer | FLT1 | Detrimental | ASO | Knockdown of Detrimental |
| | | Gene Expression | | Gene Expression |
| Cancer | HNRNPH1 | Detrimental | ASO | Knockdown of Detrimental |
| | | Gene Expression | | Gene Expression |
| Cancer | KDR | Detrimental | ASO | Knockdown of Detrimental |
| | | Gene Expression | | Gene Expression |
| Cancer | SRA1 | Detrimental | ASO | Knockdown of Detrimental |
| | | Gene Expression | | Gene Expression |
| Cancer | STAT3 | Detrimental | ASO | Knockdown of Detrimental |
| | | Gene Expression | | Gene Expression |
| Cancer | TERT | Detrimental | ASO | Knockdown of Detrimental |
| | | Gene Expression | | Gene Expression |
| Cancer | WT1 | Detrimental | ASO | Knockdown of Detrimental |
| | | Gene Expression | | Gene Expression |
| FHBL/atherosclerosis | APOB | Detrimental | ASO | Knockdown of Detrimental |
| | | Gene Expression | | Gene Expression |
| Immune-response | CD40 | Detrimental | ASO | Knockdown of Detrimental |
| | | Gene Expression | | Gene Expression |
| Inflammatory disease | TNFRSF1B | Detrimental | ASO | Knockdown of Detrimental |
| | | Gene Expression | | Gene Expression |
| Inflammatory disease | IL5RA | Detrimental | ASO | Knockdown of Detrimental |
| | | Gene Expression | | Gene Expression |
| Influenza virus | TMPRSS2 | Detrimental | ASO | Knockdown of Detrimental |
| | | Gene Expression | | Gene Expression |
| Muscle wasting diseases | MSTN | Detrimental | ASO | Knockdown of Detrimental |
| | | Gene Expression | | Gene Expression |
| Spinocerebellar | ATXN1 | Detrimental | ASO | Knockdown of Detrimental |
| ataxia type 1 | | Gene Expression | | Gene Expression |
| Duchenne muscular | DMD | RNA Reframing | ASO | RNA Reframing |
| dystrophy | | | | |
| Dystrophic | COL7A1 | RNA Reframing | ASO | RNA Reframing |
| epidermolysis bullosa | | | | |
| Miyoshi myopathy | DYSF | RNA Reframing | ASO | RNA Reframing |

| Beta-thalassemia | HBB | Splice Site Mutations |
|---|---|---|
| Alzheimer's disease/FTDP-17 Taupathies | MAPT | Deregulated Alternative Splicing |
| Spinal muscular atrophy | SMN2 | Deregulated Alternative Splicing |
| Dystrophic epidermolysis bullosa | COL7A1 | RNA Reframing |
| Familial dysautonomia | IKBKAP | Splice Site Mutations |
| Cystic fibrosis | CFTR | Cryptic Splice Sites |
| Neurofibromatosis | NF1 | Regulatory Sequence Mutations |
| Alzheimer's disease/FTDP-17 Taupathies | MAPT | Deregulated Alternative Splicing |
| Cancer | Multiple | Deregulated Alternative Splicing |
| Spinal muscular atrophy | SMN2 | Deregulated Alternative Splicing |
| Cancer | CDKN1A | Detrimental Gene Expression |
| Cancer | MYC | Detrimental Gene Expression |
| Cancer | Multiple | Detrimental Gene Expression |
| Cancer | PHB | Detrimental Gene Expression |
| Duchenne muscular dystrophy | DMD | RNA Reframing |
| Cystic Fibrosis | CFTR | Splice Site Mutations |
| Factor VII deficiency | F7 | Splice Site Mutations |
| Fanconi anemia | FANCC | Splice Site Mutations |
| Hemophilia A | F9 | Splice Site Mutations |
| Propionic acidemia | PCCA | Splice Site Mutations |
| Retinitis pigmentosa | RHO | Splice Site Mutations |
| Retinitis pigmentosa | RPGR | Splice Site Mutations |
| Spinal muscular atrophy | SMN2 | Deregulated Alternative Splicing |
| Bardet-Biedl syndrome | BBS1 | Splice Site Mutations |

| Disease | Human Target Gene | Therapeutic | Stage |
|---|---|---|---|
| Bardet-Biedl syndrome | BBS1 | U1/U6 snRNA* | Patient cells |
| Beta-thalassemia | HBB | PTM | Minigene |
| Cancer | BRCA1 | ASO | Minigene |
| Cancer | PTCH1 | ASO | Minigene |
| Cystic Fibrosis | CFTR | U1 snRNA* | Minigene |
| Duchenne muscular dystrophy | DMD | ASO | Canine model |
| Factor VII deficiency | F7 | U1 snRNA* | Minigene |
| Familial dysautonomia | IKBKAP | SM | Patients |
| Fanconi anemia | FANCC | U1 snRNA* | Patient cells |
| Hemophilia A | F9 | U1 snRNA* | Minigene |
| Propionic acidemia | PCCA | U1 snRNA* | Patient cells |
| Retinitis pigmentosa | RHO | U1 snRNA* | Minigenes |
| Retinitis pigmentosa | RPGR | U1 snRNA* | Patient cells |
| Ataxia telangiectasia | ATM | ASO | Patient cells |
| Beta-thalassemia | HBB | ASO | Mouse model |
| Cancer | BRCA2 | ASO | Minigene |
| CDG1A | PMM2 | ASO | Patient cells |
| Congenital adrenal insufficiency | CYP11A | ASO | Minigene |
| Cystic fibrosis | CFTR | ASO | Cell lines |
| Cystic fibrosis | CFTR | SM | Patient cells |
| Duchenne muscular dystrophy | DMD | ASO | Patient cells |
| Fukuyama congenital muscular | FKTN | ASO | Mouse model |
| dystrophy (FCMD) | | | |
| Growth hormone insensitivity | GHR | ASO | Minigene |
| HPABH4A | PTS | ASO | Patient cells |
| Hutchinson-Gilford progeria | LMNA | ASO | Mouse model |
| (HGPS) | | | |
| MLC1 | MLC1 | ASO | Minigene |
| Methylmalonic aciduria | MUT | ASO | Patient cells |
| Myopathy with lactic acidosis | ISCU | ASO | Patient cells |
| Myotonic dystrophy | CLC1 | ASO | Mouse model |
| Neurofibromatosis | NF1 | ASO | Patient cells |
| Niemann-Pick type C | NPC1 | ASO | Patient cells |
| Propionic acidemia | PCCB | ASO | Patient cells |
| Usher syndrome | USH1C | ASO | Mouse model |
| Afibrinogenemia | FGB | ASO | Minigene |
| Cancer | BRCA1 | ASO | In vitro |
| Propionic acidemia | PCCA | ASO | Patient cells |
| Neurofibromatosis | NF1 | SM | Patient cells |
| Ocular albinism type 1 | GRP143 | ASO | Patient cells |
| Alzheimer's disease/FTDP-17 | MAPT | ASO | Cell lines |
| Taupathies | | | |
| Alzheimer's disease/FTDP-17 | MAPT | PTM | Minigene |
| Taupathies | | | |
| Alzheimer's disease/FTDP-17 | MAPT | SM | Cell lines |
| Taupathies | | | |
| Cancer | BCL2L1 | ASO | Mouse model |
| Cancer | FGFR1 | ASO | Cell lines |
| Cancer | MCL1 | ASO | Cell lines |
| Cancer | MDM2 | ASO | Cell lines |
| Cancer | Multiple | SM | Cell lines |
| Cancer | PKM | ASO | Cell lines |
| Cancer | MST1R | ASO | Cell lines |
| Cancer | USP5 | ASO | Cell lines |
| Spinal muscular atrophy | SMN2 | ASO | Clinical |
| Spinal muscular atrophy | SMN2 | SM | Clincal trials |
| Spinal muscular atrophy | SMN2 | U1 snRNA* | Minigene |
| Spinal muscular atrophy | SMN2 | PTM | Mouse model |
| Alzheimer's disease | BACE1 | ASO | Cell lines |
| Cancer | CDKN1A | SM | Cell lines |
| Cancer | ERBB2 | ASO | Cell lines |
| Cancer | FLT1 | ASO | Mouse model |
| Cancer | HNRNPH1 | ASO | Patient cells |
| Cancer | KDR | ASO | Mouse model |
| Cancer | MYC | SM | Cell lines |
| Cancer | Multiple | SM | Clinical trials |
| | | | phase I, E7107 |
| Cancer | PHB | SM | Cell lines |
| Cancer | SRA1 | ASO | Cell lines |
| Cancer | STAT3 | ASO | Mouse model |
| Cancer | TERT | ASO | Cell lines |
| Cancer | WT1 | ASO | Cell lines |
| FHBL/atherosclerosis | APOB | ASO | Cell lines |
| Immune-response | CD40 | ASO | Cell lines |
| Inflammatory disease | TNFRSF1B | ASO | Mouse model |
| Inflammatory disease | IL5RA | ASO | Cell lines |
| Influenza virus | TMPRSS2 | ASO | Cell lines |
| Muscle wasting diseases | MSTN | ASO | Mouse model |
| Spinocerebellar ataxia type 1 | ATXN1 | ASO | Cell lines |
| Duchenne muscular dystrophy | DMD | ASO | Clinical |
| Duchenne muscular dystrophy | DMD | SM | Cell lines |
| Dystrophic epidermolysis | COL7A1 | ASO | Explants |
| bullosa | | | |
| Dystrophic epidermolysis | COL7A1 | PTM | Patient cells |
| bullosa | | | |
| Miyoshi myopathy | DYSF | ASO | Patient cells |

In some embodiments, a provided oligonucleotide composition is administered at a dose and/or frequency lower than that of an otherwise comparable reference oligonucleotide composition with comparable effect in altering the splicing of a target transcript. In some embodiments, a stereocontrolled oligonucleotide composition is administered at a dose and/or frequency lower than that of an otherwise comparable stereorandom reference oligonucleotide composition with comparable effect in altering the splicing of the target transcript. If desired, a provided composition can also be administered at higher dose/frequency due to its lower toxicities.

In some embodiments, the present disclosure recognizes that properties, e.g., activities, toxicities, etc. of oligonucleotides and compositions thereof can be optimized by chemical modifications and/or stereochemistry. In some embodiments, the present disclosure provides methods for optimizing oligonucleotide properties through chemical modifications and stereochemistry. In some embodiments, the present disclosure provides oligonucleotides and compositions and methods thereof with low toxicities. In some embodiments, the present disclosure provides oligonucleotides and compositions and methods thereof with low toxicities and enhanced activities (e.g., target-inhibition efficiency, specificity, cleavage rates, cleavage pattern, etc.). In some embodiments, the present disclosure provides oligonucleotides and compositions and methods thereof with improved protein binding profile. In some embodiments, the present disclosure provides oligonucleotides and compositions and methods thereof with improved protein binding profile and enhanced activities. In some embodiments, the present disclosure provides oligonucleotides and compositions and methods thereof with improved delivery and enhanced activities.

In some embodiments, provided oligonucleotides, compositions and methods have low toxicities, e.g., when compared to a reference composition. As widely known in the art, oligonucleotides can induce toxicities when administered to, e.g., cells, tissues, organism, etc. In some embodiments, oligonucleotides can induce undesired immune response. In some embodiments, oligonucleotide can induce complement activation. In some embodiments, oligonucleotides can induce activation of the alternative pathway of complement. In some embodiments, oligonucleotides can induce inflammation. Among other things, the complement system has strong cytolytic activity that can damages cells and should therefore be modulated to reduce potential injuries. In some embodiments, oligonucleotide-induced vascular injury is a recurrent challenge in the development of oligonucleotides for e.g., pharmaceutical use. In some embodiments, a primary source of inflammation when high doses of oligonucleotides are administered involves activation of the alternative complement cascade. In some embodiments, complement activation is a common challenge associated with phosphorothioate-containing oligonucleotides, and there is also a potential of some sequences of phosphorothioates to induce innate immune cell activation. In some embodiments, cytokine release is associated with administration of oligonucleotides. For example, in some embodiments, increases in interleukin-6 (IL-6) monocyte chemoattractant protein (MCP-1) and/or interleukin-12 (IL-12) is observed. See, e.g., Frazier, Antisense Oligonucleotide Therapies: The Promise and the Challenges from a Toxicologic Pathologist's Perspective. Toxicol Pathol., 43: 78-89, 2015; and Engelhardt, et al., Scientific and Regulatory Policy Committee Points-to-consider Paper: Drug-induced Vascular Injury Associated with Nonsmall Molecule Therapeutics in Preclinical Development: Part 2. Antisense Oligonucleotides. Toxicol Pathol. 43: 935-944, 2015.

By controlling of chemical modifications and/or stereochemistry, the present disclosure provides improved oligonucleotide compositions and methods. In some embodiments, provided oligonucleotides comprise chemical modifications. In some embodiments, provided oligonucleotides comprise base modifications, sugar modifications, internucleotidic linkage modifications, or any combinations thereof. In some embodiments, provided oligonucleotides comprise base modifications. In some embodiments, provided oligonucleotides comprise sugar modifications. In some embodiments, provided oligonucleotides comprises 2′-modifications on the sugar moieties. In some embodiments, the present disclosure demonstrates that 2′-modifications can lower toxicity. In some embodiments, provided oligonucleotides comprises one or more modified internucleotidic linkages and one or more natural phosphate linkages. In some embodiments, the present disclosure demonstrates that incorporation of one or more natural phosphate linkages into oligonucleotides comprising one or more modified internucleotidic linkages can lower toxicity. A natural phosphate linkage can be incorporated into various locations of an oligonucleotide. In some embodiments, a natural phosphate linkage is incorporated into a wing region, or a region close to the 5′- or the 3′-end. In some embodiments, a natural phosphate linkage is incorporated into the middle of an oligonucleotide. In some embodiments, a natural phosphate linkage is incorporated into a core region. In some embodiments, the present disclosure demonstrates that stereochemistry, either alone or in combination with chemical modifications, can modulate toxicity. In some embodiments, the present disclosure demonstrates that stereochemistry, either alone or in combination with chemical modifications, can modulate immune response. In some embodiments, the present disclosure demonstrates that stereochemistry, either alone or in combination with chemical modifications, can modulate complement activation. It is surprisingly found that a chirally controlled oligonucleotide composition of an individual stereoisomer can have dramatically different toxicity profile, e.g., complement activation, compared to the corresponding stereorandom composition, and/or a chirally controlled oligonucleotide composition of another individual stereoisomer. In some embodiments, the present disclosure demonstrates that stereochemistry, either alone or in combination with chemical modifications, can modulate complement activation via the alternative pathway. Example chemical modifications, stereochemistry and patterns thereof are extensively described in this disclosure, and they can be used in combinations. Example compositions and methods of are also extensively described in this disclosure. A person having ordinary skill in the art understands that methods and compositions described herein can be used to either increase or decrease immune responses, including complement activation, relative to a reference composition.

Delivery of oligonucleotides to target locations can benefit conjugation with lipids. In some embodiments, the present disclosure provides a method comprising administering a provided composition, which composition displays improved delivery as compared with a reference composition which does not comprise the lipids in the provided composition.

In some embodiments, provided oligonucleotides, compositions and methods provide improved cytoplasmatic delivery. In some embodiments, improved delivery is to a population of cells. In some embodiments, improved delivery is to a tissue. In some embodiments, improved delivery is to an organ. In some embodiments, improved delivery is to an organism. In some embodiments, improved delivery is to muscle.

In some embodiments, a reference oligonucleotide composition of a provided oligonucleotide composition is a comparable composition absence of the lipids in the provided composition. In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a chirally un-controlled (or stereorandom) composition of oligonucleotides having the same base sequence and chemical modifications. In some embodiments, a reference composition is a composition of oligonucleotides having the same base sequence but different chemical modifications. In some embodiments, a reference composition is a composition of oligonucleotides having the same base sequence, base modifications, internucleotidic linkage modifications but different sugar modifications. In some embodiments, a reference composition has fewer 2′-modified sugar modifications. In some embodiments, a reference composition is a composition of oligonucleotides having the same base sequence, base modifications, sugar modifications but different internucleotidic linkage modifications. In some embodiments, a reference composition has more internucleotidic linkage modifications. In some embodiments, a reference composition has fewer natural phosphate linkages. In some embodiments, a reference composition comprising oligonucleotides having no natural phosphate linkages. In some embodiments, a reference composition is a composition comprising a reference plurality of oligonucleotides wherein individual oligonucleotides within the reference plurality differ from one another in stereochemical structure. In some embodiments, a reference composition is a composition comprising a reference plurality of oligonucleotides, wherein at least some oligonucleotides within the reference plurality have a structure different from a structure represented by a plurality of oligonucleotides of a composition compared to the reference composition. In some embodiments, a reference composition is a composition comprising a reference plurality of oligonucleotides wherein at least some oligonucleotides within the reference plurality do not comprise a wing region and a core region. In some embodiments, a reference oligonucleotide composition comprises a reference plurality of oligonucleotides having the same common nucleotide sequence but lacking at least one of the one or more modified sugar moieties in oligonucleotides of the oligonucleotide composition compared to the reference composition. In some embodiments, a reference oligonucleotide composition comprises a reference plurality of oligonucleotides having the same common nucleotide sequence but have no modified sugar moieties. In some embodiments, a reference oligonucleotide composition comprises a reference plurality of oligonucleotides having the same common nucleotide sequence but do not comprise natural phosphate linkages. In some embodiments, a reference composition is a chirally controlled oligonucleotide composition of oligonucleotides having the same chemical modification patterns. In some embodiments, a reference composition is a chirally controlled oligonucleotide composition of another stereoisomer.

In some embodiments, provided oligonucleotides comprise one or more structural elements (e.g., modifications, stereochemistry, patterns, etc.) that oligonucleotides of the reference plurality do not all have. Such structural elements can be any one described in this disclosure.

In some embodiments, oligonucleotides of a provided composition comprise more phosphorothioate linkages than oligonucleotides of the reference composition. In some embodiments, oligonucleotides of a provided composition comprise more phosphorothioate linkages than oligonucleotides of the reference composition at the 5′-end region. In some embodiments, oligonucleotides of a provided composition comprise more phosphorothioate linkages than oligonucleotides of the reference composition at the 3′-end region. In some embodiments, oligonucleotides of a provided composition comprise more phosphorothioate linkages in a wing region than the corresponding region of oligonucleotides of the reference composition. In some embodiments, oligonucleotides of a provided composition comprise more phosphorothioate linkages in each wing region than the corresponding regions in oligonucleotides of the reference composition. In some embodiments, oligonucleotides of a provided composition comprise more Sp chiral internucleotidic linkages than oligonucleotides of the reference composition. In some embodiments, oligonucleotides of a provided composition comprise more Sp phosphorothioate linkages than oligonucleotides of the reference composition. In some embodiments, oligonucleotides of a provided composition comprise more Sp phosphorothioate linkages than oligonucleotides of the reference composition at the 5′-end region. In some embodiments, oligonucleotides of a provided composition comprise more Sp phosphorothioate linkages than oligonucleotides of the reference composition at the 3′-end region. In some embodiments, oligonucleotides of a provided composition comprise more Sp phosphorothioate linkages in a wing region than oligonucleotides of the reference composition. In some embodiments, oligonucleotides of a provided composition comprise more Sp phosphorothioate linkages in each wing region than oligonucleotides of the reference composition. In some embodiments, oligonucleotides of a provided composition comprise more modified bases than oligonucleotides of the reference composition. In some embodiments, oligonucleotides of a provided composition comprise more methylated bases than oligonucleotides of the reference composition. In some embodiments, oligonucleotides of a provided composition comprise more methylated bases than oligonucleotides of the reference composition at the 5′-end region. In some embodiments, oligonucleotides of a provided composition comprise more methylated bases than oligonucleotides of the reference composition at the 3′-end region. In some embodiments, oligonucleotides of a provided composition comprise more methylated bases than in a wing region than oligonucleotides of the reference composition. In some embodiments, oligonucleotides of a provided composition comprise more methylated bases than in each wing region than oligonucleotides of the reference composition.

In some embodiments, oligonucleotides of a provided composition comprise fewer 2′-MOE modifications than oligonucleotides of the reference composition. In some embodiments, oligonucleotides of a provided composition comprise fewer 2′-MOE modifications than oligonucleotides of the reference composition. In some embodiments, oligonucleotides of a provided composition comprise fewer 2′-MOE modifications than oligonucleotides of the reference composition at the 5′-end region. In some embodiments, oligonucleotides of a provided composition comprise fewer 2′-MOE modifications than oligonucleotides of the reference composition at the 3′-end. In some embodiments, oligonucleotides of a provided composition comprise fewer 2′-MOE modifications than in a wing region than oligonucleotides of the reference composition. In some embodiments, oligonucleotides of a provided composition comprise fewer 2′-MOE modifications than in each wing region than oligonucleotides of the reference composition. In some embodiments, individual oligonucleotides within the reference plurality differ from one another in stereochemical structure. In some embodiments, at least some oligonucleotides within the reference plurality have a structure different from a structure represented by the plurality of oligonucleotides of the composition. In some embodiments, at least some oligonucleotides within the reference plurality do not comprise a wing region and a core region. In some embodiments, the reference composition is a substantially racemic preparation of oligonucleotides that share the base sequence. In some embodiments, the reference composition is a chirally controlled oligonucleotide composition of another oligonucleotide type. In some embodiments, oligonucleotides of the reference composition comprise more phosphorothioate linkages. In some embodiments, oligonucleotides of the reference composition comprise only phosphorothioate linkages. In some embodiments, oligonucleotides of the reference composition comprise fewer modified sugar moieties. In some embodiments, oligonucleotides of the reference composition comprise fewer modified sugar moieties, wherein the modification is 2′-OR¹. In some embodiments, oligonucleotides of the reference composition comprise more modified sugar moieties. In some embodiments, oligonucleotides of the reference composition comprise more modified sugar moieties, the modification is 2′-OR¹. In some embodiments, oligonucleotides of the reference composition comprise fewer phosphorothioate linkages. In some embodiments, oligonucleotides of the reference composition have a wing, and comprise fewer phosphorothioate linkages at the wing. In some embodiments, oligonucleotides of the reference composition comprise fewer Sp phosphorothioate linkages. In some embodiments, oligonucleotides of the reference composition have a wing, and comprise fewer Sp phosphorothioate linkages at the wing. In some embodiments, oligonucleotides of the reference composition comprise more Rp phosphorothioate linkages. In some embodiments, oligonucleotides of the reference composition have a wing, and comprise more Rp phosphorothioate linkages at the wing. In some embodiments, oligonucleotides of the reference composition comprise fewer methylated bases. In some embodiments, oligonucleotides of the reference composition comprise more 2′-MOE modifications. In some embodiments, oligonucleotides of the reference composition comprise fewer natural phosphate linkages. In some embodiments, oligonucleotides of the reference composition comprise fewer natural phosphate linkages at the 5′- and/or 3′-end. In some embodiments, oligonucleotides of the reference composition comprise fewer natural phosphate linkages in a region corresponding to a wing of oligonucleotides of the first plurality. In some embodiments, oligonucleotides of a provided composition comprise natural phosphate linkages in a wing, and oligonucleotides of the reference composition comprise fewer natural phosphate linkages at the corresponding wing region. In some embodiments, oligonucleotides of a provided composition comprises natural phosphate linkages in a wing, and oligonucleotides of the reference composition comprises modified internucleotidic linkages at one or more such natural phosphate linkage locations in a wing. In some embodiments, oligonucleotides of a provided composition comprise natural phosphate linkages in a wing, and oligonucleotides of the reference composition comprises phosphorothioate linkages at one or more such natural phosphate linkage locations in a wing. In some embodiments, oligonucleotides of the reference composition comprise no natural phosphate linkages. In some embodiments, oligonucleotides of the reference composition comprise no wing-core-wing structure. In some embodiments, oligonucleotides of a provided composition comprise a 5′-end wing region comprising a natural phosphate linkage between the two nucleosides at its 3′-end, and oligonucleotides of a reference plurality do not have a natural phosphate linkage at the same position. In some embodiments, oligonucleotides of a provided composition comprise a 3′-end wing region comprising a natural phosphate linkage between the two nucleosides at its 5′-end, and oligonucleotides of a reference plurality do not have a natural phosphate linkage at the same position.

In some embodiments, oligonucleotides of a provided composition contain more 2′-F modifications than oligonucleotides of a reference composition. In some embodiments, oligonucleotides of a provided composition contain more 2′-F modifications in a wing region. In some embodiments, oligonucleotides of a provided composition contain more 2′-F modifications in each wing region.

In some embodiments, provided chirally controlled oligonucleotide compositions comprises oligonucleotides of one oligonucleotide type. In some embodiments, provided chirally controlled oligonucleotide compositions comprises oligonucleotides of only one oligonucleotide type. In some embodiments, provided chirally controlled oligonucleotide compositions has oligonucleotides of only one oligonucleotide type. In some embodiments, provided chirally controlled oligonucleotide compositions comprises oligonucleotides of two or more oligonucleotide types. In some embodiments, using such compositions, provided methods can target more than one target. In some embodiments, a chirally controlled oligonucleotide composition comprising two or more oligonucleotide types targets two or more targets. In some embodiments, a chirally controlled oligonucleotide composition comprising two or more oligonucleotide types targets two or more mismatches. In some embodiments, a single oligonucleotide type targets two or more targets, e.g., mutations. In some embodiments, a target region of oligonucleotides of one oligonucleotide type comprises two or more “target sites” such as two mutations or SNPs.

In some embodiments, oligonucleotides in a provided chirally controlled oligonucleotide composition optionally comprise modified bases or sugars. In some embodiments, a provided chirally controlled oligonucleotide composition does not have any modified bases or sugars. In some embodiments, a provided chirally controlled oligonucleotide composition does not have any modified bases. In some embodiments, oligonucleotides in a provided chirally controlled oligonucleotide composition comprise modified bases and sugars. In some embodiments, oligonucleotides in a provided chirally controlled oligonucleotide composition comprise a modified base. In some embodiments, oligonucleotides in a provided chirally controlled oligonucleotide composition comprise a modified sugar. Modified bases and sugars for oligonucleotides are widely known in the art, including but not limited in those described in the present disclosure. In some embodiments, a modified base is 5-mC. In some embodiments, a modified sugar is a 2′-modified sugar. Suitable 2′-modification of oligonucleotide sugars are widely known by a person having ordinary skill in the art. In some embodiments, 2′-modifications include but are not limited to 2′-OR¹, wherein R¹ is not hydrogen. In some embodiments, a 2′-modification is 2′-OR¹, wherein R¹ is optionally substituted C₁₋₆ aliphatic. In some embodiments, a 2′-modification is 2′-MOE. In some embodiments, a modification is 2′-halogen. In some embodiments, a modification is 2′-F. In some embodiments, modified bases or sugars may further enhance activity, stability and/or selectivity of a chirally controlled oligonucleotide composition, whose common pattern of backbone chiral centers provides unexpected activity, stability and/or selectivity.

In some embodiments, a provided chirally controlled oligonucleotide composition does not have any modified sugars. In some embodiments, a provided chirally controlled oligonucleotide composition does not have any 2′-modified sugars. In some embodiments, the present disclosure surprising found that by using chirally controlled oligonucleotide compositions, modified sugars are not needed for stability, activity, and/or control of cleavage patterns. Furthermore, in some embodiments, the present disclosure surprisingly found that chirally controlled oligonucleotide compositions of oligonucleotides without modified sugars deliver better properties in terms of stability, activity, turn-over and/or control of cleavage patterns. For example, in some embodiments, it is surprising found that chirally controlled oligonucleotide compositions of oligonucleotides having no modified sugars dissociates much faster from cleavage products and provide significantly increased turn-over than compositions of oligonucleotides with modified sugars.

As discussed in detail herein, the present disclosure provides, among other things, a chirally controlled oligonucleotide composition, meaning that the composition contains a plurality of oligonucleotides of at least one type. Each oligonucleotide molecule of a particular “type” is comprised of preselected (e.g., predetermined) structural elements with respect to: (1) base sequence; (2) pattern of backbone linkages; (3) pattern of backbone chiral centers; and (4) pattern of backbone P-modification moieties. In some embodiments, provided oligonucloetide compositions contain oligonucleotides that are prepared in a single synthesis process. In some embodiments, provided compositions contain oligonucloetides having more than one chiral configuration within a single oligonucleotide molecule (e.g., where different residues along the oligonucleotide have different stereochemistry); in some such embodiments, such oligonucleotides may be obtained in a single synthesis process, without the need for secondary conjugation steps to generate individual oligonucleotide molecules with more than one chiral configuration.

Oligonucleotide compositions as provided herein can be used as agents for modulating a number of cellular processes and machineries, including but not limited to, transcription, translation, immune responses, epigenetics, etc. In addition, oligonucleotide compositions as provided herein can be used as reagents for research and/or diagnostic purposes. One of ordinary skill in the art will readily recognize that the present disclosure herein is not limited to particular use but is applicable to any situations where the use of synthetic oligonucleotides is desirable. Among other things, provided compositions are useful in a variety of therapeutic, diagnostic, agricultural, and/or research applications.

In some embodiments, provided oligonucleotide compositions comprise oligonucleotides and/or residues thereof that include one or more structural modifications as described in detail herein. In some embodiments, provided oligonucleotide compositions comprise oligonucleoties that contain one or more nucleic acid analogs. In some embodiments, provided oligonucleotide compositions comprise oligonucleotides that contain one or more artificial nucleic acids or residues, including but not limited to: peptide nucleic acids (PNA), Morpholino and locked nucleic acids (LNA), glycon nucleic acids (GNA), threose nucleic acids (TNA), Xeno nucleic acids (ZNA), and any combination thereof.

In any of the embodiments, the disclosure is useful for oligonucleotide-based modulation of gene expression, immune response, etc. Accordingly, stereo-defined, oligonucleotide compositions of the disclosure, which contain oligonucleotides of predetermined type (i.e., which are chirally controlled, and optionally chirally pure), can be used in lieu of conventional stereo-random or chirally impure counterparts. In some embodiments, provided compositions show enhanced intended effects and/or reduced unwanted side effects. Certain embodiments of biological and clinical/therapeutic applications of the disclosure are discussed explicitly below.

Various dosing regimens can be utilized to administer provided chirally controlled oligonucleotide compositions. In some embodiments, multiple unit doses are administered, separated by periods of time. In some embodiments, a given composition has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second (or subsequent) dose amount that is same as or different from the first dose (or another prior dose) amount. In some embodiments, a dosing regimen comprises administering at least one unit dose for at least one day. In some embodiments, a dosing regimen comprises administering more than one dose over a time period of at least one day, and sometimes more than one day. In some embodiments, a dosing regimen comprises administering multiple doses over a time period of at least week. In some embodiments, the time period is at least 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per week for more than one week. In some embodiments, a dosing regimen comprises administering one dose per week for 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose every two weeks for more than two week period. In some embodiments, a dosing regimen comprises administering one dose every two weeks over a time period of 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per month for one month. In some embodiments, a dosing regimen comprises administering one dose per month for more than one month. In some embodiments, a dosing regimen comprises administering one dose per month for 2, 3, 4, 5, 6, 7, 8, 9, 10, 11, 12, or more months. In some embodiments, a dosing regimen comprises administering one dose per week for about 10 weeks. In some embodiments, a dosing regimen comprises administering one dose per week for about 20 weeks. In some embodiments, a dosing regimen comprises administering one dose per week for about 30 weeks. In some embodiments, a dosing regimen comprises administering one dose per week for 26 weeks. In some embodiments, a chirally controlled oligonucleotide composition is administered according to a dosing regimen that differs from that utilized for a chirally uncontrolled (e.g., stereorandom) oligonucleotide composition of the same sequence, and/or of a different chirally controlled oligonucleotide composition of the same sequence. In some embodiments, a chirally controlled oligonucleotide composition is administered according to a dosing regimen that is reduced as compared with that of a chirally uncontrolled (e.g., sterorandom) oligonucleotide composition of the same sequence in that it achieves a lower level of total exposure over a given unit of time, involves one or more lower unit doses, and/or includes a smaller number of doses over a given unit of time. In some embodiments, a chirally controlled oligonucleotide composition is administered according to a dosing regimen that extends for a longer period of time than does that of a chirally uncontrolled (e.g., stereorandom) oligonucleotide composition of the same sequence Without wishing to be limited by theory, Applicant notes that in some embodiments, the shorter dosing regimen, and/or longer time periods between doses, may be due to the improved stability, bioavailability, and/or efficacy of a chirally controlled oligonucleotide composition. In some embodiments, a chirally controlled oligonucleotide composition has a longer dosing regimen compared to the corresponding chirally uncontrolled oligonucleotide composition. In some embodiments, a chirally controlled oligonucleotide composition has a shorter time period between at least two doses compared to the corresponding chirally uncontrolled oligonucleotide composition. Without wishing to be limited by theory, Applicant notes that in some embodiments longer dosing regimen, and/or shorter time periods between doses, may be due to the improved safety of a chirally controlled oligonucleotide composition.

In some embodiments, with their low toxicity, provided oligonucleotides and compositions can be administered in higher dosage and/or with higher frequency. In some embodiments, with their improved delivery (and other properties), provided compositions can be administered in lower dosages and/or with lower frequency to achieve biological effects, for example, clinical efficacy.

A single dose can contain various amounts of oligonucleotides. In some embodiments, a single dose can contain various amounts of a type of chirally controlled oligonucleotide, as desired suitable by the application. In some embodiments, a single dose contains about 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 or more (e.g., about 350, 400, 450, 500, 550, 600, 650, 700, 750, 800, 850, 900, 950, 1000 or more) mg of a type of chirally controlled oligonucleotide. In some embodiments, a single dose contains about 1 mg of a type of chirally controlled oligonucleotide. In some embodiments, a single dose contains about 5 mg of a type of chirally controlled oligonucleotide. In some embodiments, a single dose contains about 10 mg of a type of chirally controlled oligonucleotide. In some embodiments, a single dose contains about 15 mg of a type of chirally controlled oligonucleotide. In some embodiments, a single dose contains about 20 mg of a type of chirally controlled oligonucleotide. In some embodiments, a single dose contains about 50 mg of a type of chirally controlled oligonucleotide. In some embodiments, a single dose contains about 100 mg of a type of chirally controlled oligonucleotide. In some embodiments, a single dose contains about 150 mg of a type of chirally controlled oligonucleotide. In some embodiments, a single dose contains about 200 mg of a type of chirally controlled oligonucleotide. In some embodiments, a single dose contains about 250 mg of a type of chirally controlled oligonucleotide. In some embodiments, a single dose contains about 300 mg of a type of chirally controlled oligonucleotide. In some embodiments, a chirally controlled oligonucleotide is administered at a lower amount in a single dose, and/or in total dose, than a chirally uncontrolled oligonucleotide. In some embodiments, a chirally controlled oligonucleotide is administered at a lower amount in a single dose, and/or in total dose, than a chirally uncontrolled oligonucleotide due to improved efficacy. In some embodiments, a chirally controlled oligonucleotide is administered at a higher amount in a single dose, and/or in total dose, than a chirally uncontrolled oligonucleotide. In some embodiments, a chirally controlled oligonucleotide is administered at a higher amount in a single dose, and/or in total dose, than a chirally uncontrolled oligonucleotide due to improved safety.

A provided oligonucleotide composition as used herein may comprise single stranded and/or multiply stranded oligonucleotides. In some embodiments, single-stranded oligonucleotides contain self-complementary portions that may hybridize under relevant conditions so that, as used, even single-stranded oligonucleotides may have at least partially double-stranded character. In some embodiments, an oligonucleotide included in a provided composition is single-stranded, double-stranded, or triple-stranded. In some embodiments, an oligonucleotide included in a provided composition comprises a single-stranded portion and a multiple-stranded portion within the oligonucleotide. In some embodiments, as noted above, individual single-stranded oligonucleotides can have double-stranded regions and single-stranded regions.

In some embodiments, provided compositions include one or more oligonucleotides fully or partially complementary to strand of: structural genes, genes control and/or termination regions, and/or self-replicating systems such as viral or plasmid DNA. In some embodiments, provided compositions include one or more oligonucleotides that are or act as siRNAs or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, Ul adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, morpholinos, G-quadruplex (RNA and DNA), antiviral oligonucleotides, and decoy oligonucleotides.

In some embodiments, provided compositions include one or more hybrid (e.g., chimeric) oligonucleotides. In the context of the present disclosure, the term “hybrid” broadly refers to mixed structural components of oligonucloetides. Hybrid oliogonucleotides may refer to, for example, (1) an oligonucleotide molecule having mixed classes of nucleotides, e.g., part DNA and part RNA within the single molecule (e.g., DNA-RNA); (2) complementary pairs of nucleic acids of different classes, such that DNA:RNA base pairing occurs either intramolecularly or intermolecularly; or both; (3) an oligonucleotide with two or more kinds of the backbone or internucleotide linkages.

In some embodiments, provided compositions include one or more oligonucleotide that comprises more than one classes of nucleic acid residues within a single molecule. For example, in any of the embodiments described herein, an oligonucleotide may comprise a DNA portion and an RNA portion. In some embodiments, an oligonucleotide may comprise a unmodified portion and modified portion.

Provided oligonucleotide compositions can include oligonucleotides containing any of a variety of modifications, for example as described herein. In some embodiments, particular modifications are selected, for example, in light of intended use. In some embodiments, it is desirable to modify one or both strands of a double-stranded oligonucleotide (or a double-stranded portion of a single-stranded oligonucleotide). In some embodiments, the two strands (or portions) include different modifications. In some embodiments, the two strands include the same modifications. One of skill in the art will appreciate that the degree and type of modifications enabled by methods of the present disclosure allow for numerous permutations of modifications to be made. Example such modifications are described herein and are not meant to be limiting.

The phrase “antisense strand” as used herein, refers to an oligonucleotide that is substantially or 100% complementary to a target sequence of interest. The phrase “antisense strand” includes the antisense region of both oligonucleotides that are formed from two separate strands, as well as unimolecular oligonucleotides that are capable of forming hairpin or dumbbell type structures. The terms “antisense strand” and “guide strand” are used interchangeably herein.

The phrase “sense strand” refers to an oligonucleotide that has the same nucleoside sequence, in whole or in part, as a target sequence such as a messenger RNA or a sequence of DNA. The terms “sense strand” and “passenger strand” are used interchangeably herein.

By “target sequence” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant. In some embodiments, a target sequence is associated with a disease or disorder.

By “specifically hybridizable” and “complementary” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LIT pp. 123-133; Freier et al., 1986, Proc. Nat. Acad. Sci. USA 83:9373-9377; Turner et al., 1987, J. Am. Chem. Soc. 109:3783-3785)

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, e.g., under physiological conditions in the case of in vivo assays or therapeutic treatment, or in the case of in vitro assays, under conditions in which the assays are performed. In some embodiments, non-target sequences differ from corresponding target sequences by at least 5 nucleotides.

When used as therapeutics, a provided oligonucleotide is administered as a pharmaceutical composition. In some embodiments, the pharmaceutical composition comprises a therapeutically effective amount of a provided oligonucleotide comprising, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In some embodiments, in provided compositions provided oligonucleotides may exist as salts, preferably pharmaceutically acceptable salts, e.g., sodium salts, ammonium salts, etc. In some embodiments, a salt of a provided oligonucleotide comprises two or more cations, for example, in some embodiments, up to the number of negatively charged acidic groups (e.g., phosphate, phosphorothioate, etc.) in an oligonucleotide. In another embodiment, the pharmaceutical composition is formulated for intravenous injection, oral administration, buccal administration, inhalation, nasal administration, topical administration, ophthalmic administration or otic administration. In further embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

### Pharmaceutical Compositions

When used as therapeutics, a provided oligonucleotide or oligonucleotide composition described herein is administered as a pharmaceutical composition. In some embodiments, the pharmaceutical composition comprises a therapeutically effective amount of a provided oligonucleotides, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled oligonucleotide, or composition thereof, in admixture with a pharmaceutically acceptable excipient. One of skill in the art will recognize that the pharmaceutical compositions include the pharmaceutically acceptable salts of the chirally controlled oligonucleotide, or composition thereof, described above.

A variety of supramolecular nanocarriers can be used to deliver nucleic acids. Example nanocarriers include, but are not limited to liposomes, cationic polymer complexes and various polymeric. Complexation of nucleic acids with various polycations is another approach for intracellular delivery; this includes use of PEGlyated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, polymer micelles, quantum dots and lipoplexes. In some embodiments, an oligonucleotide is conjugated to another molecular.

Additional nucleic acid delivery strategies are known in addition to the example delivery strategies described herein.

In therapeutic and/or diagnostic applications, the compounds of the disclosure can be formulated for a variety of modes of administration, including systemic and topical or localized administration. Techniques and formulations generally may be found in Remington, The Science and Practice of Pharmacy, (20th ed. 2000).

Provided oligonucleotides, and compositions thereof, are effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions, described herein, within the central nervous system may be achieved with intraparenchymal administration, intrathecal administration, or intracerebroventricular administration.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC50) by a factor of 20, 25, 30, 35, 40, 45 or 50. In certain embodiments, the pharmaceutical agent in an antisense compound as further described herein. In certain embodiments, the targeted tissue is brain tissue. In certain embodiments the targeted tissue is striatal tissue. In certain embodiments, decreasing EC50 is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with oligonucleotides of this disclosure. For example, chemotherapeutic agents or other anti-proliferative agents may be combined with the oligonucleotides of this disclosure to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

The function and advantage of these and other embodiments of the present disclosure will be more fully understood from the examples described below. The following examples are intended to illustrate the benefits of the present disclosure, but do not exemplify the full scope of the disclosure.

### Targeting Components

In some embodiments, a provided composition further comprises a targeting component. A targeting component can be either conjugated or not conjugated to a lipid or a biologically active agent. In some embodiments, a targeting component is conjugated to a biologically active agent. In some embodiments, a biologically active agent is conjugated to both a lipid and a targeting component. As described in here, in some embodiments, a biologically active agent is a provided oligonucleotide. Thus, in some embodiments, a provided oligonucleotide composition further comprises, besides a lipid and oligonucleotides, a target elements. Various targeting components can be used in accordance with the present disclosure, e.g., lipids, antibodies, peptides, carbohydrates, etc. In some embodiments, provided oligonucleotides have the structure of A^{c}-[-L^{LD}-(R^{LD})ₐ]_{b}. In some embodiments, provided oligonucleotides have the structure of [(A^{c})ₐ-L^{LD}]_{b}-R^{LD}. In some embodiments, L^{LD}, R^{LD} combinations of L^{LD} and R^{LD}, or -[-L^{LD}-(R^{LD})ₐ]_{b} comprises one or more targeting components.

In some embodiments, a targeting component interacts with a protein on the surface of targeted cells. In some embodiments, such interaction facilitates internalization into targeted cells. In some embodiments, a targeting component comprises a sugar moiety. In some embodiments, a targeting component comprises a polypeptide moiety. In some embodiments, a targeting component comprises an antibody. In some embodiments, a targeting component is an antibody. In some embodiments, a targeting component comprises an inhibitor. In some embodiments, a targeting component is a moiety from a small molecule inhibitor. In some embodiments, an inhibitor is an inhibitor of a protein on the surface of targeted cells. In some embodiments, an inhibitor is a carbonic anhydrase inhibitor. In some embodiments, an inhibitor is a carbonic anhydrase inhibitor expressed on the surface of target cells. In some embodiments, a carbonic anhydrase is I, II, III, IV, V, VI, VII, VIII, IX, X, XI, XII, XIII, XIV, XV or XVI. In some embodiments, a carbonic anhydrase is membrane bound. In some embodiments, a carbonic anhydrase is IV, IX, XII or XIV. In some embodiments, an inhibitor is for IV, IX, XII and/or XIV. In some embodiments, an inhibitor is a carbonic anhydrase III inhibitor. In some embodiments, an inhibitor is a carbonic anhydrase IV inhibitor. In some embodiments, an inhibitor is a carbonic anhydrase IX inhibitor. In some embodiments, an inhibitor is a carbonic anhydrase XII inhibitor. In some embodiments, an inhibitor is a carbonic anhydrase XIV inhibitor. In some embodiments, an inhibitor comprises or is a sulfonamide (e.g., those described in Supuran, CT. Nature Rev Drug Discover 2008, 7, 168-181, which sulfonamides are incorporated herein by reference). In some embodiments, an inhibitor is a sulfonamide. In some embodiments, targeted cells are muscle cells.

In some embodiments, a targeting component is R^{LD} as defined and described in the present disclosure. In some embodiments, the present disclosure provides oligonucleotides comprising R^{LD}. In some embodiments, the present disclosure provides oligonucleotide compositions comprising oligonucleotides comprising R^{LD}. In some embodiments, the present disclosure provides oligonucleotide compositions comprising a first plurality of oligonucleotides comprising R^{LD}. In some embodiments, the present disclosure provides chirally controlled oligonucleotide compositions of oligonucleotides comprising R^{LD}. In some embodiments, R^{LD} comprises or is

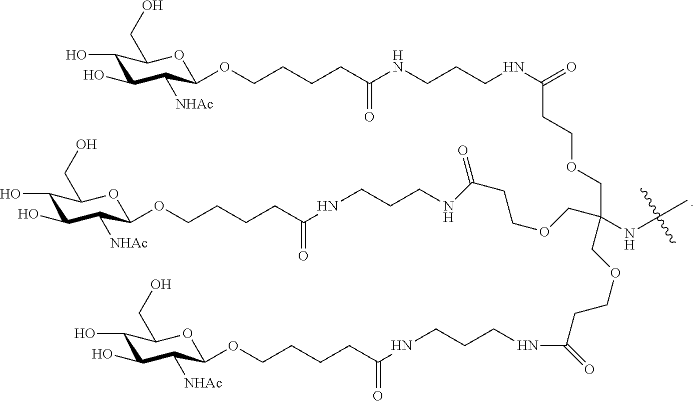

In some embodiments, R^{LD} comprises or is

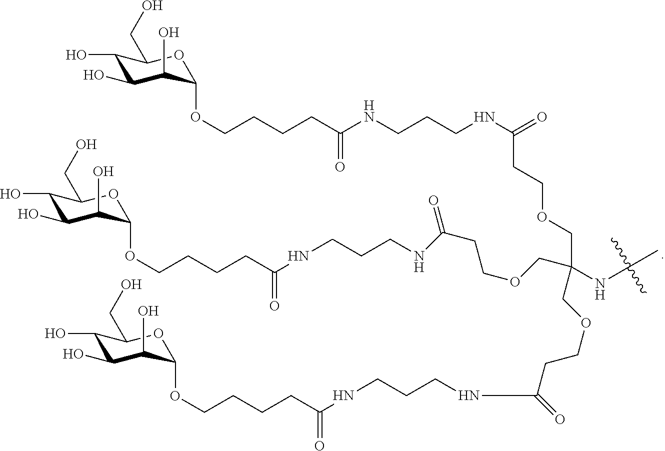

In some embodiments, R^{LD} comprises or is

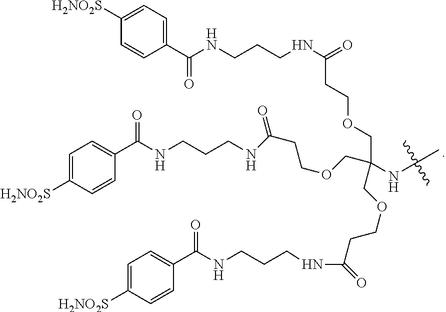 [SMILES: *NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{LD} comprises or is

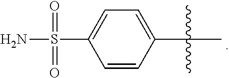 [SMILES: *c1ccc(S(N)(=O)=O)cc1]

In some embodiments, R^{LD} comprises or is

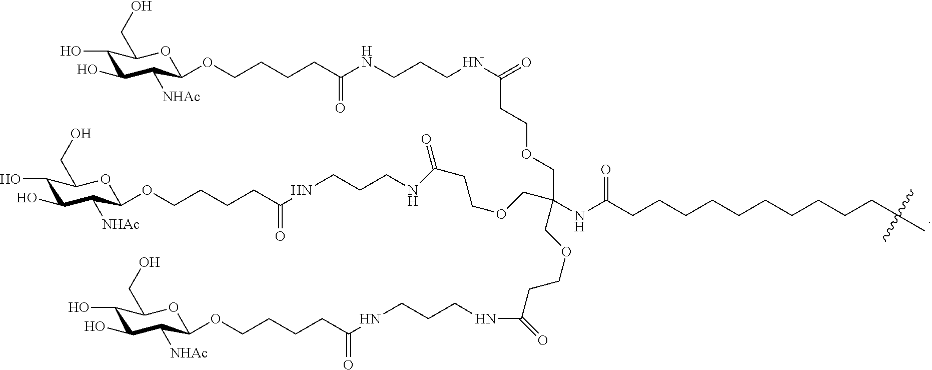

In some embodiments, R^{LD} comprises or is

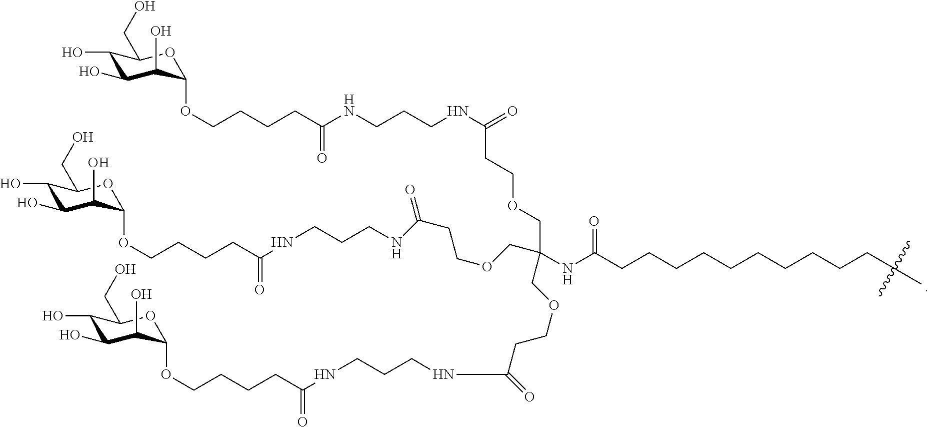

In some embodiments, R^{LD} comprises or is

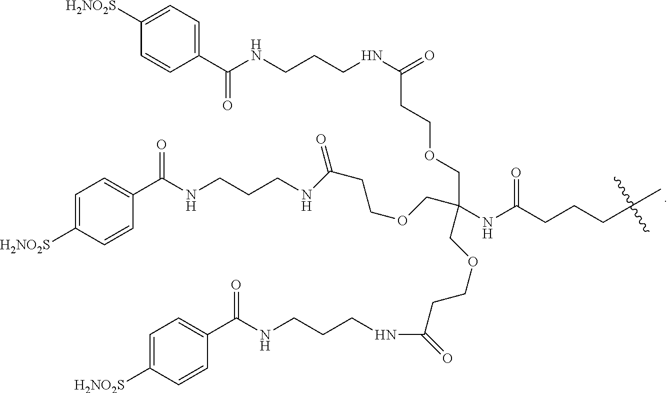 [SMILES: *CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{LD} comprises or is

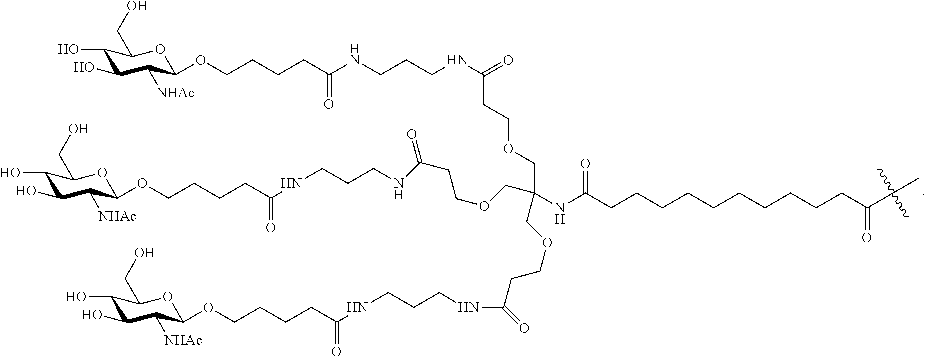

In some embodiments, R^{LD} comprises or is

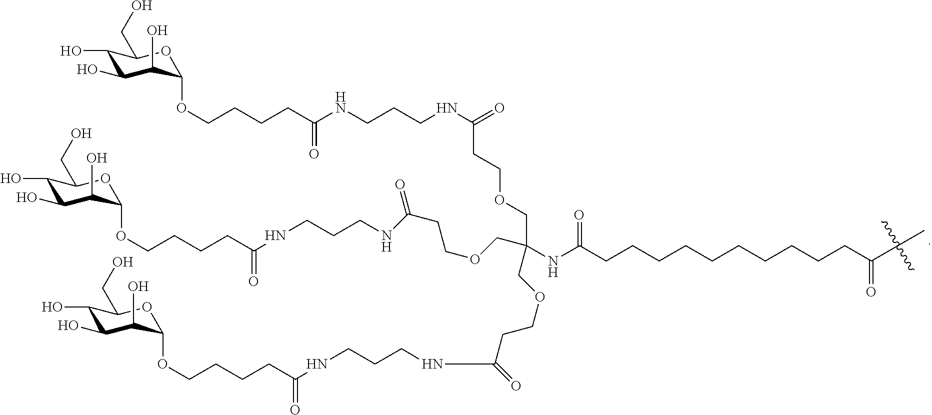

In some embodiments, R^{LD} comprises or is

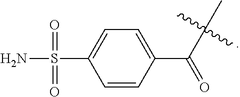 [SMILES: *C(=O)c1ccc(S(N)(=O)=O)cc1]

In some embodiments, R^{LD} comprises or is

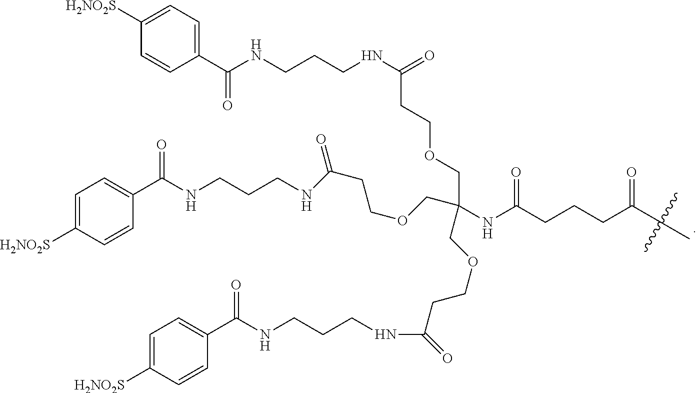 [SMILES: *C(=O)CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{LD} comprises or is

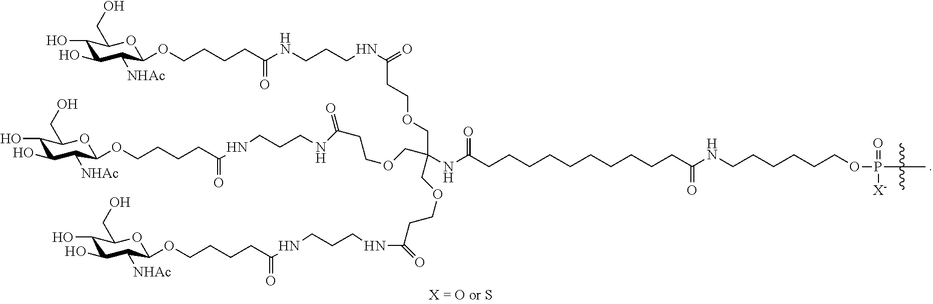

In some embodiments, R^{LD} comprises or is

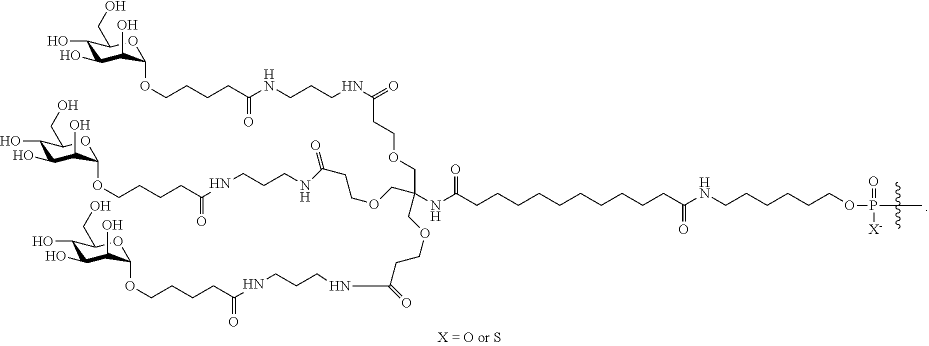

In some embodiments, R^{LD} comprises or is

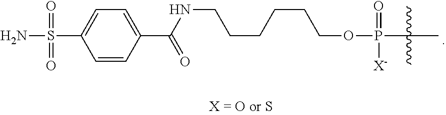

In some embodiments, R^{LD} comprises or is

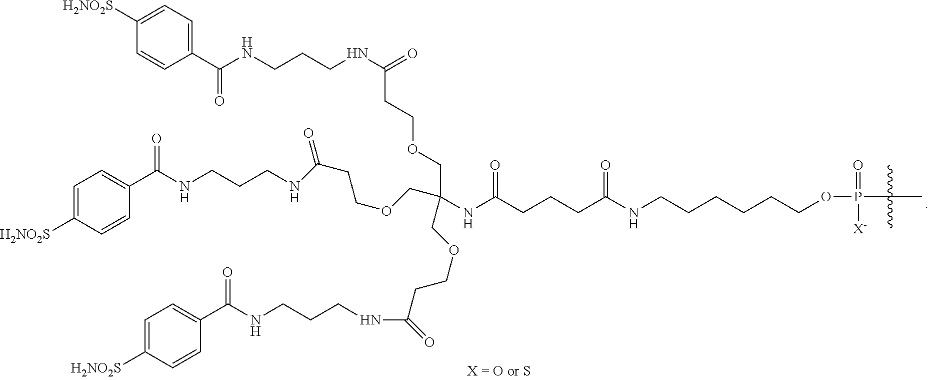

In some embodiments, X is O. In some embodiments, X is S.

In some embodiments, the present disclosure provides technologies (e.g., reagents, methods, etc.) for conjugating various moieties to oligonucleotide chains. In some embodiments, the present disclosure provides technologies for conjugating targeting component to oligonucleotide chains. In some embodiments, the present disclosure provides acids comprising targeting components for conjugation, e.g., R^{LD}—COOH. In some embodiments, the present disclosure provides linkers for conjugation, e.g., L^{LD}. A person having ordinary skill in the art understands that many known and widely practiced technologies can be utilized for conjugation with oligonucleotide chains in accordance with the present disclosure. In some embodiments, a provided acid is

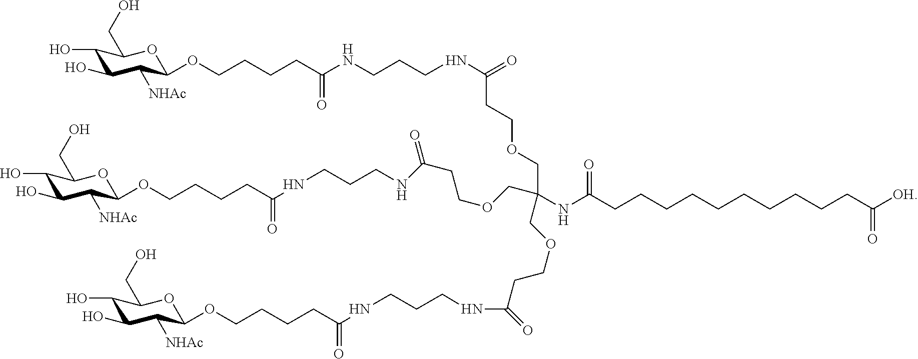

In some embodiments, a provided acid is

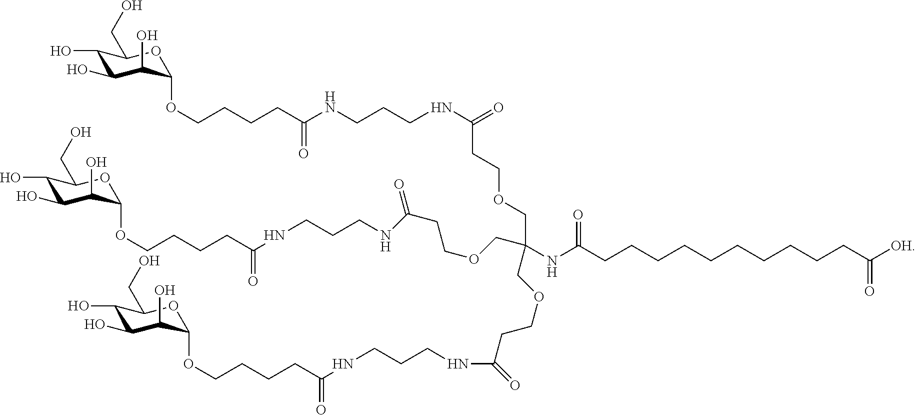

In some embodiments, a provided acid is

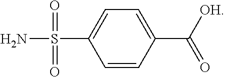 [SMILES: NS(=O)(=O)c1ccc(C(=O)O)cc1]

In some embodiments, a provided acid is

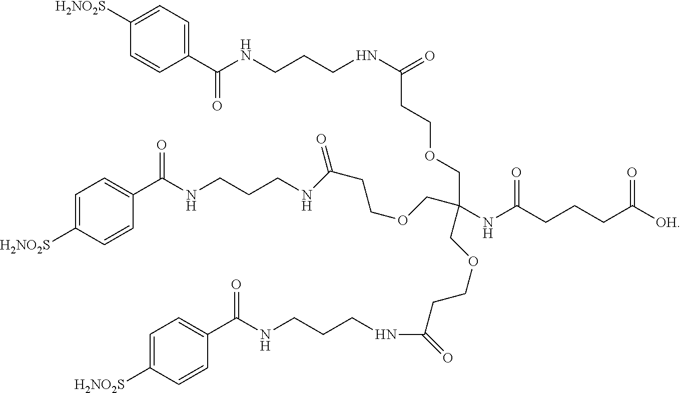 [SMILES: NS(=O)(=O)c1ccc(C(=O)NCCCNC(=O)CCOCC(COCCC(=O)NCCCNC(=O)c2ccc(S(N)(=O)=O)cc2)(COCCC(=O)NCCCNC(=O)c2ccc(S(N)(=O)=O)cc2)NC(=O)CCCC(=O)O)cc1]

In some embodiments, the present disclosure provides methods and reagents for preparing such acids.

In some embodiments, provided compounds, e.g., reagents, products (e.g., oligonucleotides, amidites, etc.) etc. are at least 10%, 20%, 30%, 40%, 50%, 60%, 70%, 75%, 80%, 81%, 82%, 83%, 84%, 85%, 86%, 87%, 88%, 89%, 90%, 91%, 92%, 93%, 94%, 95%, 96%, 97%, 97% or 99% pure. In some embodiments, the purity is at least 50%. In some embodiments, the purity is at least 75%. In some embodiments, the purity is at least 80%. In some embodiments, the purity is at least 85%. In some embodiments, the purity is at least 90%. In some embodiments, the purity is at least 95%. In some embodiments, the purity is at least 96%. In some embodiments, the purity is at least 97%. In some embodiments, the purity is at least 98%. In some embodiments, the purity is at least 99%.

Target components can be incorporated into provided technologies through many types of methods in accordance with the present disclosure. In some embodiments, target components are physically mixed with provided oligonucleotides to form provided compositions. In some embodiments, target components are chemically conjugated with oligonucleotides. In some embodiments, target components are chemically conjugated with oligonucleotides through a linker, for example, L^{LD}.

In some embodiments, provided compositions comprise two or more target components. In some embodiments, provided oligonucleotides comprise two or more conjugated target components. In some embodiments, the two or more conjugated target components are the same. In some embodiments, the two or more conjugated target components are different. In some embodiments, provided oligonucleotides comprise no more than one target component. In some embodiments, oligonucleotides of a provided composition comprise different types of conjugated target components. In some embodiments, oligonucleotides of a provided composition comprise the same type of target components.

Target components can be conjugated to oligonucleotides optionally through linkers. Various types of linkers in the art can be utilized in accordance of the present disclosure. In some embodiments, a linker comprise a phosphate group, which can, for example, be used for conjugating target components through chemistry similar to those employed in oligonucleotide synthesis. In some embodiments, a linker comprises an amide, ester, or ether group. In some embodiments, a linker has the structure of -L-. Target components can be conjugated through either the same or different linkers compared to lipids.

Target components, optionally through linkers, can be conjugated to oligonucleotides at various suitable locations. In some embodiments, target components are conjugated through the 5′-OH group. In some embodiments, target components are conjugated through the 3′-OH group. In some embodiments, target components are conjugated through one or more sugar moieties. In some embodiments, target components are conjugated through one or more bases. In some embodiments, target components are incorporated through one or more internucleotidic linkages. In some embodiments, an oligonucleotide may contain multiple conjugated target components which are independently conjugated through its 5′-OH, 3′-OH, sugar moieties, base moieties and/or internucleotidic linkages. Target components and lipids can be conjugated either at the same, neighboring and/or separated locations. In some embodiments, a target component is conjugated at one end of an oligonucleotide, and a lipid is conjugated at the other end.

### Example Uses

In some embodiments, the present disclosure encompasses the use of a composition comprising a lipid and a biologically active agent. In some embodiments, the present disclosure provides methods for delivering a biologically active agent to a target location comprising administering a provided composition. In some embodiments, a provided method delivers a biologically active agent into a cell. In some embodiments, a provided method delivers a biologically active agent into a muscle cell. In some embodiments, a provided method delivers a biologically active agent into a cell within a tissue. In some embodiments, a provided method delivers a biologically active agent into a cell within an organ. In some embodiments, a provided method delivers a biologically active agent into a cell within a subject, comprising administering to the subject a provided composition. In some embodiments, a provided method delivers a biologically active agent into cytoplasm. In some embodiments, a provided method delivers a biologically active agent into nucleus.

In some embodiments, the present disclosure pertains to methods related to the delivery of a biologically active agent to a muscle cell or tissue, or a muscle cell or tissue in a mammal (e.g., a human subject), which method pertains to a use of a composition comprising a biological agent and a lipid.

### Biologically Active Agent: A Nucleic Acid

In some embodiments, the present disclosure pertains to compositions and methods related to a composition comprising a biologically active agent and a lipid comprising a C₁₀-C₄₀ linear, saturated or partially unsaturated, aliphatic chain. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group. In some embodiments, the present disclosure pertains to compositions and methods related to delivery of biologically active agents, wherein the compositions comprise a biologically active agent a lipid. In various embodiments, the lipid is selected from: lauric acid, myristic acid, palmitic acid, stearic acid, oleic acid, linoleic acid, alpha-linolenic acid, gamma-linolenic acid, docosahexaenoic acid (DHA or cis-DHA), turbinaric acid and dilinoleyl.

In some embodiments, a biologically active agent is a nucleic acid.

In some embodiments, a nucleic acid is an oligonucleotide, an antisense oligonucleotide, an RNAi agent, a miRNA, splice switching oligonucleotide (SSO), immunomodulatory nucleic acid, an aptamer, a ribozyme, a Piwi-interacting RNA (piRNA), a small nucleolar RNA (snoRNA), a mRNA, a lncRNA, a ncRNA, an antigomir (e.g., an antagonist to a miRNA, lncRNA, ncRNA or other nucleic acid), a plasmid, a vector, or a portion thereof.

In some embodiments, a nucleic acid is an oligonucleotide.

In some embodiments, the present disclosure pertains to: an oligonucleotide composition comprising a plurality of oligonucleotides, which share: 1) a common base sequence; 2) a common pattern of backbone linkages; and 3) a common pattern of backbone phosphorus modifications; wherein one or more oligonucleotides of the plurality are individually conjugated to a lipid. In some embodiments, the present disclosure pertains to: a chirally controlled oligonucleotide composition comprising a plurality of oligonucleotides, which share: 1) a common base sequence; 2) a common pattern of backbone linkages; and 3) a common pattern of backbone phosphorus modifications; wherein: the composition is chirally controlled in that the plurality of oligonucleotides share the same stereochemistry at one or more chiral internucleotidic linkages; one or more oligonucleotides of the plurality are individually conjugated to a lipid; and one or more oligonucleotides of the plurality are optionally and individually conjugated to a targeting compound or moiety. In some embodiments, the oligonucleotide is a splice-switching oligonucleotide. In some embodiments, the biologically active agent is an oligonucleotide capable of skipping or mediating skipping of an exon in a gene related to a muscle-related disease or disorder. In some embodiments, the biologically active agent is an oligonucleotide capable of skipping or mediating skipping of an exon in the dystrophin gene. In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, the sequence of the oligonucleotide comprises or consists of the sequence of any splice-switching oligonucleotide disclosed herein (e.g., in Table 4A).

In some embodiments, the plurality of oligonucleotides share the same stereochemistry at five or more chiral internucleotidic linkages. In some embodiments, the plurality of oligonucleotides share the same stereochemistry at ten or more chiral internucleotidic linkages. In some embodiments, the plurality of oligonucleotides share the same stereochemistry at each of the chiral internucleotidic linkages so that they share a common pattern of backbone chiral centers. In some embodiments, one or more oligonucleotides of the plurality are independently conjugated to a lipid through a 5′-OH on the oligonucleotide. In some embodiments, one or more oligonucleotides of the plurality are independently conjugated to a lipid through a 3′-OH on the oligonucleotide. In some embodiments, each oligonucleotide of the plurality is individually conjugated to a lipid. In some embodiments, each oligonucleotide of the plurality is individually conjugated to the same lipid. In some embodiments, the present disclosure pertains to: a composition comprising a biologically active agent and a lipid, wherein the agent is any agent disclosed herein, and wherein the lipid is any lipid disclosed herein. In some embodiments, each oligonucleotide of the plurality is individually conjugated to the same lipid at the same location. In some embodiments, a lipid is conjugated to an oligonucleotide through a linker. In some embodiments, one or more oligonucleotides of the plurality are independently conjugated to a targeting compound or moiety. In some embodiments, one or more oligonucleotides of the plurality are independently conjugated to a lipid and a targeting compound or moiety. In some embodiments, one or more oligonucleotides of the plurality are independently conjugated to a lipid at one end and a targeting compound or moiety at the other. In some embodiments, oligonucleotides of the plurality share the same chemical modification patterns. In some embodiments, oligonucleotides of the plurality share the same chemical modification patterns comprising one or more base modifications. In some embodiments, oligonucleotides of the plurality share the same chemical modification patterns comprising one or more sugar modifications. In some embodiments, the sequence of the oligonucleotide(s) comprises or consists of the sequence of any splice-switching oligonucleotide disclosed herein (e.g., in Table 4A).

In some embodiments, the plurality of oligonucleotides share a common pattern of sugar modification, which comprises 3 or more 2′-F. In some embodiments, the plurality of oligonucleotides share a common pattern of sugar modification, which comprises 3 or more consecutive 2′-F. In some embodiments, the plurality of oligonucleotides share a common pattern of sugar modification, which comprises 3 or more consecutive 2′-F within the 10 nucleotide at the 5′-end. In some embodiments, the plurality of oligonucleotides share a common pattern of sugar modification, which comprises 3 or more 2′-F within the 10 nucleotide at the 5′-end. In some embodiments, the plurality of oligonucleotides share a common pattern of sugar modification, which comprises 3 or more consecutive 2′-F at the 5′-end. In some embodiments, the plurality of oligonucleotides share a common pattern of sugar modification, which comprises 5 or more consecutive 2′-F within the first 10 nucleotide at the 3′-end. In some embodiments, the plurality of oligonucleotides share a common pattern of sugar modification, which comprises 5 or more 2′-F within the 10 nucleotide at the 3′-end. In some embodiments, the plurality of oligonucleotides share a common pattern of sugar modification, which comprises 7 or more consecutive 2′-F at the 3′-end. In some embodiments, the plurality of oligonucleotides share a common pattern of sugar modification, which comprises 3 or more consecutive 2′-F at the 5′-end, 3 or more consecutive 2′-F at the 3′-end, and 3 or more 2′-OR between the 5′-end 2′-F and the 3′-end 2′-F modifications. In some embodiments, the plurality of oligonucleotides share a common pattern of sugar modification, which comprises 3 or more 2′-F at the 5′-end, 3 or more 2′-F at the 3′-end, and 3 or more 2′-OR between the 5′-end 2′-F and the 3′-end 2′-F modifications. In some embodiments, the plurality of oligonucleotides share a common pattern of sugar modification, which comprises 5 or more 2′-F within the 10 nucleotides at the 5′-end. In some embodiments, the plurality of oligonucleotides share a common pattern of sugar modification, which comprises 3 or more consecutive 2′-F at the 5′-end. In some embodiments, the plurality of oligonucleotides share a common pattern of sugar modification, which comprises 7 or more 2′-F within the 10 nucleotides at the 3′-end. In some embodiments, the plurality of oligonucleotides share a common pattern of sugar modification, which comprises 5 or more consecutive 2′-F within the 10 nucleotides at the 3′-end. In some embodiments, the plurality of oligonucleotides share a common pattern of sugar modification, which comprises 7 or more consecutive 2′-F at the 3′-end. In some embodiments, the plurality of oligonucleotides comprises a 5′-wing-core-wing-3′ structure, wherein each wing region independently comprises 3 to 10 nucleosides, and the core region independently comprises 3 to 10 nucleosides. In some embodiments, the sequence of the oligonucleotide(s) comprises or consists of the sequence of any oligonucleotide disclosed herein (e.g., in Table 4A).

In some embodiments, the present disclosure pertains to: a method of delivering an oligonucleotide to a muscle cell or tissue in a human subject, comprising: (a) Providing a composition of any one of the preceding claims; and (b) Administering the composition to the human subject such that the oligonucleotide is delivered to a muscle cell or tissue in the subject. In some embodiments, the sequence of the oligonucleotide(s) comprises or consists of the sequence of any oligonucleotide disclosed herein (e.g., in Table 4A).

In some embodiments, the common base sequence is capable of hybridizing with a transcript in a muscle cell, which transcript contains a mutation that is linked to a muscle disease, or whose level, activity and/or distribution is linked to a muscle disease. In some embodiments, the common base sequence is capable of hybridizing with a transcript in a muscle cell, and the composition is characterized in that when it is contacted with the transcript in a transcript splicing system, splicing of the transcript is altered relative to that observed under reference conditions selected from the group consisting of absence of the composition, presence of a reference composition, and combinations thereof. In some embodiments, the common base sequence is capable of hybridizing with a transcript in a cell. In some embodiments, a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14). In some embodiments, the common base sequence hybridizes with a transcript of dystrophin. In some embodiments, the common base sequence hybridizes with a transcript of dystrophin, and the composition increases the production of one or more functional or partially functional proteins encoded by dystrophin. In some embodiments, the sequence of the oligonucleotide(s) comprises or consists of the sequence of any oligonucleotide disclosed herein (e.g., in Table 4A).

In some embodiments, the oligonucleotide or oligonucleotides is or are splice switching oligonucleotide or oligonucleotides. In some embodiments, the sequence of the oligonucleotide(s) comprises or consists of the sequence of any oligonucleotide disclosed herein (e.g., in Table 4A).

In some embodiments, the present disclosure pertains to:

A method for inhibiting expression of a gene in a muscle cell or tissue in a mammal comprising preparing a composition comprising a lipid and an oligonucleotide (as a non-limiting example, a SSO) and administering the composition to the mammal.

A method of treating a disease that is caused by the over-expression of one or several proteins in a muscle cell or tissue in a subject, said method comprising the administration of a composition comprising a lipid and an oligonucleotide (as a non-limiting example, a SSO).

A method of treating a disease that is caused by a reduced, suppressed or missing expression of one or several proteins in a subject, said method comprising the administration of a composition comprising a lipid and an oligonucleotide (as a non-limiting example, a SSO).

A method for treating a sign and/or symptom of a disease, disorder, or condition related to a muscle-related disorder or disease in a subject by providing a composition comprising a lipid and an oligonucleotide (as a non-limiting example, a SSO) and administering a therapeutically effective amount of the composition to the subject.

A method of administering a biologically active agent to a subject in need thereof, comprising steps of providing a composition comprising a biologically active agent and a lipid, and administering the composition to the subject, wherein the biologically active compound is an oligonucleotide (as a non-limiting example, a SSO), and wherein the lipid is any lipid disclosed herein.

A method of treating a disease in a subject, the method comprising steps of providing a composition comprising a biologically active agent and a lipid, and administering a therapeutically effective amount of the composition to the subject, wherein the biologically active compound is an oligonucleotide (as a non-limiting example, a SSO), and wherein the lipid is any lipid disclosed herein, and wherein the disease is any disease disclosed herein.

A method for inhibiting expression of a gene in a muscle cell or tissue in a mammal, wherein the gene is related to a muscle-related disease or disorder, the method comprising steps of preparing a composition comprising a lipid and an oligonucleotide (as a non-limiting example, a SSO) and administering the composition to the mammal.

A method of treating a disease that is caused by the over-expression of one or several proteins in a muscle cell or tissue in a subject, said method comprising the administration of a composition comprising a lipid and an oligonucleotide (as a non-limiting example, a SSO).

A method of treating a disease that is caused by a reduced, suppressed or missing expression of one or several proteins in a muscle in a subject, said method comprising the administration of a composition comprising a lipid and an oligonucleotide (as a non-limiting example, a SSO).

A method for treating a sign and/or symptom of a disease, disorder, or condition related to a muscle-related disorder or disease in a subject by providing a composition comprising a lipid and an oligonucleotide (as a non-limiting example, a SSO) and administering a therapeutically effective amount of the composition to the subject.

A method of administering a biologically active agent to a subject in need thereof, comprising steps of providing a composition comprising a biologically active agent and a lipid, and administering the composition to the subject, wherein the biologically active compound is an oligonucleotide (as a non-limiting example, a SSO), and wherein the lipid is any lipid disclosed herein.

A method of treating a muscle-related disease or disorder in a subject, the method comprising steps of providing a composition comprising a biologically active agent and a lipid, and administering a therapeutically effective amount of the composition to the subject, wherein the biologically active compound is an oligonucleotide (as a non-limiting example, a SSO), and wherein the lipid is any lipid disclosed herein.

A method for skipping an exon in a gene in a muscle cell or tissue in a mammal, the method comprising steps of preparing a composition comprising a lipid and a splice-switching oligonucleotide and administering the composition to the mammal.

A method of treating a disease related to an exon comprising a mutation in a gene, said method comprising the administration of a composition comprising a lipid and a splice-switching oligonucleotide, wherein the splice-switching oligonucleotide is capable of skipping or mediating skipping the exon comprising the mutation. In some embodiments, the disease is a muscle-related disease.

A method of treating a disease that is caused by a mutation in an exon in a gene, said method comprising the administration of a composition comprising a lipid and an oligonucleotide, wherein the oligonucleotide is capable of skipping or mediating skipping the exon comprising the mutation. In some embodiments, the disease is a muscle-related disease.

A method for treating a sign and/or symptom of a disease, disorder, or condition related to a muscle-related disorder or disease in a subject by providing a composition comprising a lipid and an oligonucleotide (as a non-limiting example, a SSO) and administering a therapeutically effective amount of the composition to the subject.

A method of administering a biologically active agent to a subject in need thereof, comprising steps of providing a composition comprising a biologically active agent and a lipid, and administering the composition to the subject, wherein the oligonucleotide is capable of skipping or mediating skipping an exon comprising a mutation, and wherein the lipid is any lipid disclosed herein.

A method of treating a muscle-related disease or disorder in a subject, wherein the disease or disorder is related to an exon comprising a mutation in a gene, the method comprising steps of providing a composition comprising an oligonucleotide and a lipid, and administering a therapeutically effective amount of the composition to the subject, wherein the oligonucleotide is capable of skipping or mediating skipping the exon comprising the mutation, and wherein the lipid is any lipid disclosed herein.

A method for skipping an exon in the dystrophin gene in a muscle cell or tissue in a mammal, the method comprising steps of preparing a composition comprising a lipid and a splice-switching oligonucleotide and administering the composition to the mammal.

A method of treating a disease related to an exon comprising a mutation in the dystrophin gene, said method comprising the administration of a composition comprising a lipid and a splice-switching oligonucleotide, wherein the splice-switching oligonucleotide is capable of skipping or mediating skipping the exon comprising the mutation.

A method of treating a disease that is caused by a mutation in an exon in the dystrophin gene, said method comprising the administration of a composition comprising a lipid and an oligonucleotide, wherein the oligonucleotide is capable of skipping or mediating skipping the exon comprising the mutation.

A method for treating a sign and/or symptom of a disease, disorder, or condition related to a muscle-related disorder or disease in a subject by providing a composition comprising a lipid and an oligonucleotide (as a non-limiting example, a SSO) and administering a therapeutically effective amount of the composition to the subject.

A method of administering a biologically active agent to a subject in need thereof, comprising steps of providing a composition comprising a biologically active agent and a lipid, and administering the composition to the subject, wherein the oligonucleotide is capable of skipping or mediating skipping the exon comprising the mutation, and wherein the lipid is any lipid disclosed herein.

A method of treating Duchenne muscular dystrophy in a subject, wherein the Duchenne muscular dystrophy is related to a mutation in an exon in the dystrophin gene, the method comprising steps of providing a composition comprising an oligonucleotide and a lipid, and administering a therapeutically effective amount of the composition to the subject, wherein the oligonucleotide is capable of skipping or mediating skipping the exon comprising the mutation, and wherein the lipid is any lipid disclosed herein.

A method for skipping an exon in the dystrophin gene in a muscle cell or tissue in a mammal, the method comprising steps of preparing a composition comprising a lipid and a splice-switching oligonucleotide and administering the composition to the mammal, wherein the lipid is any lipid disclosed herein, and wherein the sequence of the splice-switching oligonucleotide comprises or consists of the sequence of any splice-switching oligonucleotide disclosed herein (e.g., in Table 4A).

A method of treating a disease related to an exon comprising a mutation in the dystrophin gene, said method comprising the administration of a composition comprising a lipid and a splice-switching oligonucleotide, wherein the splice-switching oligonucleotide is capable of skipping or mediating skipping the exon comprising the mutation, wherein the lipid is any lipid disclosed herein, and wherein the sequence of the splice-switching oligonucleotide comprises or consists of the sequence of any splice-switching oligonucleotide disclosed herein (e.g., in Table 4A).

A method of treating a disease that is caused by a mutation in an exon in the dystrophin gene, said method comprising the administration of a composition comprising a lipid and an oligonucleotide, wherein the oligonucleotide is capable of skipping or mediating skipping the exon comprising the mutation, wherein the lipid is any lipid disclosed herein, and wherein the oligonucleotide comprises or consists of the sequence of any splice-switching oligonucleotide disclosed herein (e.g., in Table 4A).

A method for treating a sign and/or symptom of a disease, disorder, or condition related to a muscle-related disorder or disease in a subject by providing a composition comprising a lipid and an oligonucleotide (as a non-limiting example, a SSO) and administering a therapeutically effective amount of the composition to the subject, wherein the lipid is any lipid disclosed herein, and wherein the sequence of the oligonucleotide comprises or consists of the sequence of any splice-switching oligonucleotide disclosed herein (e.g., in Table 4A).

A method of administering a biologically active agent to a subject in need thereof, comprising steps of providing a composition comprising a biologically active agent and a lipid, and administering the composition to the subject, wherein the oligonucleotide is capable of skipping or mediating skipping the exon comprising the mutation, and wherein the lipid is any lipid disclosed herein, wherein the lipid is any lipid disclosed herein, and wherein the sequence of the oligonucleotide comprises or consists of the sequence of any splice-switching oligonucleotide disclosed herein (e.g., in Table 4A).

A method of treating Duchenne muscular dystrophy in a subject, wherein the Duchenne muscular dystrophy is related to a mutation in an exon in the dystrophin gene, the method comprising steps of providing a composition comprising an oligonucleotide and a lipid, and administering a therapeutically effective amount of the composition to the subject, wherein the oligonucleotide is capable of skipping or mediating skipping the exon comprising the mutation, and wherein the lipid is any lipid disclosed herein, and wherein the oligonucleotide comprises or consists of the sequence of any splice-switching oligonucleotide disclosed herein (e.g., in Table 4A).

In some individuals with muscular dystrophy, an exon in the dystrophin gene comprises a mutation; in many cases, this mutation causes a frameshift, which can lead to a premature stop codon. This prematurely terminated dystrophin is not full-length and thus cannot perform all the necessary functions of this protein. In some treatments for muscular dystrophy, an oligonucleotide (e.g., a splice-switching oligonucleotide) is capable of skipping or causing the skipping of one or more exons comprising a mutation; this allows the expression of a shortened dystrophin gene product, which lacks the portion corresponding to the skipped exon(s), but is otherwise functional. A non-limiting example of muscular dystrophy is Duchenne muscular dystrophy (DMD). A non-limiting example of an exon comprising a mutation causing a frameshift mutation and a premature stop is exon 51 of the dystrophin gene.

In some embodiments, a biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of exon 51, 45, 53 or 44 in the dystrophin gene.

Various oligonucleotides are listed in Table 4A. Many of these are capable of skipping or mediating skipping of exon 51 of the human dystrophin gene, as shown in data presented in U.S. Pat. Application No. 62/239,839, filed Oct. 9, 2015, which is incorporated by reference in its entirety; and in data shown here.

Various oligonucleotides particularly capable of mediating skipping of exon 51 of human dystrophin include: WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2533, among others. Thus, any composition or method described herein can comprise a biologically active agent, wherein the biologically active agent is selected from: WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2533, or any other nucleic acid disclosed herein (including, but not limited to, those listed in Table 4A).

In some embodiments, a provided oligonucleotide is selected from those presented in the tables in the present disclosure, wherein the oligonucleotide is conjugated to a lipid and optionally a target component.

TABLE 4A
Example Oligonucleotides.

| WAVE ID | Base Sequence | SEQ ID NO: | Description | SEQ ID NO: | Stereochemistry¹ | Notes | Target/Program |
|---|---|---|---|---|---|---|---|
| ONT- | UCAAGGAAGA | 205 | mU*SmC*SmA*SmA*SmG*SmG*SmA | 957 | SSSSSSSSSSSSSS | Chiral version of | DMD |
| 395 | UGGCAUUUCU | | *SmA*SmG*SmA*SmU*SmG*SmG*Sm | | SSSSS | PRO051 (Drisapersen) | |
| | | | C*SmA*SmU*SmU*SmU*SmC*SmU | | | | |
| WV-942 | UCAAGGAAGA | 206 | mU*mC*mA*mA*mG*mG*mA*mA*m | 958 | XXXXXXXXXX | PRO051 (Drisapersen) | DMD |
| | UGGCAUUUCU | | G*mA*mU*mG*mG*mC*mA*mU*mU* | | XXXXXXXXX | | |
| | | | mU*mC*mU | | | | |
| WV-943 | GGCCAAACCUC | 207 | mG*mG*mC*mC*mA*mA*mA*mC*m | 959 | XXXXXXXXXX | Exon 23 control | DMD |
| | GGCUUACCU | | C*mU*mC*mG*mG*mC*mU*mU*mA* | | XXXXXXXXX | | |
| | | | mC*mC*mU | | | | |
| WV- | CUCCAACAUCA | 208 | mC*mU*mC*mC*mA*mA*mC*mA*m | 960 | XXXXXXXXXX | eteplirsen-all-2′-Me | DMD |
| 2165 | AGGAAGAUGG | | U*mC*mA*mA*mG*mG*mA*mA*mG* | | XXXXXXXXXX | 30mer | |
| | CAUUUCUAG | | mA*mU*mG*mG*mC*mA*mU*mU*m | | XXXXXXXXX | | |
| | | | U*mC*mU*mA*mG | | | | |
| WV- | ACCAGAGUAA | 209 | mA*mC*mC*mA*mG*mA*mG*mU*m | 961 | XXXXXXXXXX | 25-mer 2′-OMethyl | DMD |
| 2179 | CAGUCUGAGU | | A*mA*mC*mA*mG*mU*mC*mU*mG* | | XXXXXXXXXX | | |
| | AGGAG | | mA*mG*mU*mA*mG*mG*mA*mG | | XXXX | | |
| WV- | CACCAGAGUA | 210 | mC*mA*mC*mC*mA*mG*mA*mG*m | 962 | XXXXXXXXXX | 25-mer 2′-OMethyl | DMD |
| 2180 | ACAGUCUGAG | | U*mA*mA*mC*mA*mG*mU*mC*mU* | | XXXXXXXXXX | | |
| | UAGGA | | mG*mA*mG*mU*mA*mG*mG*mA | | XXXX | | |
| WV- | UCACCAGAGU | 211 | mU*mC*mA*mC*mC*mA*mG*mA*m | 963 | XXXXXXXXXX | 25-mer 2′-OMethyl | DMD |
| 2181 | AACAGUCUGA | | G*mU*mA*mA*mC*mA*mG*mU*mC* | | XXXXXXXXXX | | |
| | GUAGG | | mU*mG*mA*mG*mU*mA*mG*mG | | XXXX | | |
| WV- | GUCACCAGAG | 212 | mG*mU*mC*mA*mC*mC*mA*mG*m | 964 | XXXXXXXXXX | 25-mer 2′-OMethyl | DMD |
| 2182 | UAACAGUCUG | | A*mG*mU*mA*mA*mC*mA*mG*mU* | | XXXXXXXXXX | | |
| | AGUAG | | mC*mU*mG*mA*mG*mU*mA*mG | | XXXX | | |
| WV- | GUUGUGUCAC | 213 | mG*mU*mU*mG*mU*mG*mU*mC*m | 965 | XXXXXXXXXX | 25-mer 2′-OMethyl | DMD |
| 2183 | CAGAGUAACA | | A*mC*mC*mA*mG*mA*mG*mU*mA* | | XXXXXXXXXX | | |
| | GUCUG | | mA*mC*mA*mG*mU*mC*mU*mG | | XXXX | | |
| WV- | GGUUGUGUCA | 214 | mG*mG*mU*mU*mG*mU*mG*mU*m | 966 | XXXXXXXXXX | 25-mer 2′-OMethyl | DMD |
| 2184 | CCAGAGUAAC | | C*mA*mC*mC*mA*mG*mA*mG*mU* | | XXXXXXXXXX | | |
| | AGUCU | | mA*mA*mC*mA*mG*mU*mC*mU | | XXXX | | |
| WV- | AGGUUGUGUC | 215 | mA*mG*mG*mU*mU*mG*mU*mG*m | 967 | XXXXXXXXXX | 25-mer 2′-OMethyl | DMD |
| 2185 | ACCAGAGUAA | | U*mC*mA*mC*mC*mA*mG*mA*mG* | | XXXXXXXXXX | | |
| | CAGUC | | mU*mA*mA*mC*mA*mG*mU*mC | | XXXX | | |
| WV- | CAGGUUGUGU | 216 | mC*mA*mG*mG*mU*mU*mG*mU*m | 968 | XXXXXXXXXX | 25-mer 2′-OMethyl | DMD |
| 2186 | CACCAGAGUA | | G*mU*mC*mA*mC*mC*mA*mG*mA* | | XXXXXXXXXX | | |
| | ACAGU | | mG*mU*mA*mA*mC*mA*mG*mU | | XXXX | | |
| WV- | ACAGGUUGUG | 217 | mA*mC*mA*mG*mG*mU*mU*mG*m | 969 | XXXXXXXXXX | 25-mer 2′-OMethyl | DMD |
| 2187 | UCACCAGAGU | | U*mG*mU*mC*mA*mC*mC*mA*mG* | | XXXXXXXXXX | | |
| | AACAG | | mA*mG*mU*mA*mA*mC*mA*mG | | XXXX | | |
| WV- | CCACAGGUUG | 218 | mC*mC*mA*mC*mA*mG*mG*mU*m | 970 | XXXXXXXXXX | 25-mer 2′-OMethyl | DMD |
| 2188 | UGUCACCAGA | | U*mG*mU*mG*mU*mC*mA*mC*mC* | | XXXXXXXXXX | | |
| | GUAAC | | mA*mG*mA*mG*mU*mA*mA*mC | | XXXX | | |
| WV- | ACCACAGGUU | 219 | mA*mC*mC*mA*mC*mA*mG*mG*m | 971 | XXXXXXXXXX | 25-mer 2′-OMethyl | DMD |
| 2189 | GUGUCACCAG | | U*mU*mG*mU*mG*mU*mC*mA*mC* | | XXXXXXXXXX | | |
| | AGUAA | | mC*mA*mG*mA*mG*mU*mA*mA | | XXXX | | |
| WV- | AACCACAGGU | 220 | mA*mA*mC*mC*mA*mC*mA*mG*m | 972 | XXXXXXXXXX | 25-mer 2′-OMethyl | DMD |
| 2190 | UGUGUCACCA | | G*mU*mU*mG*mU*mG*mU*mC*mA* | | XXXXXXXXXX | | |
| | GAGUA | | mC*mC*mA*mG*mA*mG*mU*mA | | XXXX | | |
| WV- | UAACCACAGG | 221 | mU*mA*mA*mC*mC*mA*mC*mA*m | 973 | XXXXXXXXXX | 25-mer 2′-OMethyl | DMD |
| 2191 | UUGUGUCACC | | G*mG*mU*mU*mG*mU*mG*mU*mC* | | XXXXXXXXXX | | |
| | AGAGU | | mA*mC*mC*mA*mG*mA*mG*mU | | XXXX | | |
| WV- | GUAACCACAG | 222 | mG*mU*mA*mA*mC*mC*mA*mC*m | 974 | XXXXXXXXXX | 25-mer 2′-OMethyl | DMD |
| 2192 | GUUGUGUCAC | | A*mG*mG*mU*mU*mG*mU*mG*mU | | XXXXXXXXXX | | |
| | CAGAG | | *mC*mA*mC*mC*mA*mG*mA*mG | | XXXX | | |
| WV- | AGUAACCACA | 223 | mA*mG*mU*mA*mA*mC*mC*mA*m | 975 | XXXXXXXXXX | 25-mer 2′-OMethyl | DMD |
| 2193 | GGUUGUGUCA | | C*mA*mG*mG*mU*mU*mG*mU*mG* | | XXXXXXXXXX | | |
| | CCAGA | | mU*mC*mA*mC*mC*mA*mG*mA | | XXXX | | |
| WV- | UAGUAACCAC | 224 | mU*mA*mG*mU*mA*mA*mC*mC*m | 976 | XXXXXXXXXX | 25-mer 2′-OMethyl | DMD |
| 2194 | AGGUUGUGUC | | A*mC*mA*mG*mG*mU*mU*mG*mU* | | XXXXXXXXXX | | |
| | ACCAG | | mG*mU*mC*mA*mC*mC*mA*mG | | XXXX | | |
| WV- | UUAGUAACCA | 225 | mU*mU*mA*mG*mU*mA*mA*mC*m | 977 | XXXXXXXXXX | 25-mer 2′-OMethyl | DMD |
| 2195 | CAGGUUGUGU | | C*mA*mC*mA*mG*mG*mU*mU*mG* | | XXXXXXXXXX | | |
| | CACCA | | mU*mG*mU*mC*mA*mC*mC*mA | | XXXX | | |
| WV- | CUUAGUAACC | 226 | mC*mU*mU*mA*mG*mU*mA*mA*m | 978 | XXXXXXXXXX | 25-mer 2′-OMethyl | DMD |
| 2196 | ACAGGUUGUG | | C*mC*mA*mC*mA*mG*mG*mU*mU* | | XXXXXXXXXX | | |
| | UCACC | | mG*mU*mG*mU*mC*mA*mC*mC | | XXXX | | |
| WV- | CCUUAGUAACC | 227 | mC*mC*mU*mU*mA*mG*mU*mA*m | 979 | XXXXXXXXXX | 25-mer 2′-OMethyl | DMD |
| 2197 | ACAGGUUGUG | | A*mC*mC*mA*mC*mA*mG*mG*mU* | | XXXXXXXXXX | | |
| | UCAC | | mU*mG*mU*mG*mU*mC*mA*mC | | XXXX | | |
| WV- | UCCUUAGUAA | 228 | mU*mC*mC*mU*mU*mA*mG*mU*m | 980 | XXXXXXXXXX | 25-mer 2′-OMethyl | DMD |
| 2198 | CCACAGGUUG | | A*mA*mC*mC*mA*mC*mA*mG*mG* | | XXXXXXXXXX | | |
| | UGUCA | | mU*mU*mG*mU*mG*mU*mC*mA | | XXXX | | |
| WV- | GUUUCCUUAG | 229 | mG*mU*mU*mU*mC*mC*mU*mU*m | 981 | XXXXXXXXXX | 25-mer 2′-OMethyl | DMD |
| 2199 | UAACCACAGG | | A*mG*mU*mA*mA*mC*mC*mA*mC* | | XXXXXXXXXX | | |
| | UUGUG | | mA*mG*mG*mU*mU*mG*mU*mG | | XXXX | | |
| WV- | AGUUUCCUUA | 230 | mA*mG*mU*mU*mU*mC*mC*mU*m | 982 | XXXXXXXXXX | 25-mer 2′-OMethyl | DMD |
| 2200 | GUAACCACAG | | U*mA*mG*mU*mA*mA*mC*mC*mA* | | XXXXXXXXXX | | |
| | GUUGU | | mC*mA*mG*mG*mU*mU*mG*mU | | XXXX | | |
| WV- | CAGUUUCCUU | 231 | mC*mA*mG*mU*mU*mU*mC*mC*m | 983 | XXXXXXXXXX | 25-mer 2′-OMethyl | DMD |
| 2201 | AGUAACCACA | | U*mU*mA*mG*mU*mA*mA*mC*mC* | | XXXXXXXXXX | | |
| | GGUUG | | mA*mC*mA*mG*mG*mU*mU*mG | | XXXX | | |
| WV- | GCAGUUUCCU | 232 | mG*mC*mA*mG*mU*mU*mU*mC*m | 984 | XXXXXXXXXX | 25-mer 2′-OMethyl | DMD |
| 2202 | UAGUAACCAC | | C*mU*mU*mA*mG*mU*mA*mA*mC* | | XXXXXXXXXX | | |
| | AGGUU | | mC*mA*mC*mA*mG*mG*mU*mU | | XXXX | | |
| WV- | GGCAGUUUCC | 233 | mG*mG*mC*mA*mG*mU*mU*mU*m | 985 | XXXXXXXXXX | 25-mer 2′-OMethyl | DMD |
| 2203 | UUAGUAACCA | | C*mC*mU*mU*mA*mG*mU*mA*mA* | | XXXXXXXXXX | | |
| | CAGGU | | mC*mC*mA*mC*mA*mG*mG*mU | | XXXX | | |
| WV- | UGGCAGUUUC | 234 | mU*mG*mG*mC*mA*mG*mU*mU*m | 986 | XXXXXXXXXX | 25-mer 2′-OMethyl | DMD |
| 2204 | CUUAGUAACC | | U*mC*mC*mU*mU*mA*mG*mU*mA* | | XXXXXXXXXX | | |
| | ACAGG | | mA*mC*mC*mA*mC*mA*mG*mG | | XXXX | | |
| WV- | AUGGCAGUUU | 235 | mA*mU*mG*mG*mC*mA*mG*mU*m | 987 | XXXXXXXXXX | 25-mer 2′-OMethyl | DMD |
| 2205 | CCUUAGUAACC | | U*mU*mC*mC*mU*mU*mA*mG*mU* | | XXXXXXXXXX | | |
| | ACAG | | mA*mA*mC*mC*mA*mC*mA*mG | | XXXX | | |
| WV- | AGAUGGCAGU | 236 | mA*mG*mA*mU*mG*mG*mC*mA*m | 988 | XXXXXXXXXX | 25-mer 2′-OMethyl | DMD |
| 2206 | UUCCUUAGUA | | G*mU*mU*mU*mC*mC*mU*mU*mA* | | XXXXXXXXXX | | |
| | ACCAC | | mG*mU*mA*mA*mC*mC*mA*mC | | XXXX | | |
| WV- | GAGAUGGCAG | 237 | mG*mA*mG*mA*mU*mG*mG*mC*m | 989 | XXXXXXXXXX | 25-mer 2′-OMethyl | DMD |
| 2207 | UUUCCUUAGU | | A*mG*mU*mU*mU*mC*mC*mU*mU* | | XXXXXXXXXX | | |
| | AACCA | | mA*mG*mU*mA*mA*mC*mC*mA | | XXXX | | |
| WV- | GGAGAUGGCA | 238 | mG*mG*mA*mG*mA*mU*mG*mG*m | 990 | XXXXXXXXXX | 25-mer 2′-OMethyl | DMD |
| 2208 | GUUUCCUUAG | | C*mA*mG*mU*mU*mU*mC*mC*mU* | | XXXXXXXXXX | | |
| | UAACC | | mU*mA*mG*mU*mA*mA*mC*mC | | XXXX | | |
| WV- | UGGAGAUGGC | 239 | mU*mG*mG*mA*mG*mA*mU*mG*m | 991 | XXXXXXXXXX | 25-mer 2′-OMethyl | DMD |
| 2209 | AGUUUCCUUA | | G*mC*mA*mG*mU*mU*mU*mC*mC* | | XXXXXXXXXX | | |
| | GUAAC | | mU*mU*mA*mG*mU*mA*mA*mC | | XXXX | | |
| WV- | UUGGAGAUGG | 240 | mU*mU*mG*mG*mA*mG*mA*mU*m | 992 | XXXXXXXXXX | 25-mer 2′-OMethyl | DMD |
| 2210 | CAGUUUCCUU | | G*mG*mC*mA*mG*mU*mU*mU*mC* | | XXXXXXXXXX | | |
| | AGUAA | | mC*mU*mU*mA*mG*mU*mA*mA | | XXXX | | |
| WV- | UUUGGAGAUG | 241 | mU*mU*mU*mG*mG*mA*mG*mA*m | 993 | XXXXXXXXXX | 25-mer 2′-OMethyl | DMD |
| 2211 | GCAGUUUCCU | | U*mG*mG*mC*mA*mG*mU*mU*mU* | | XXXXXXXXXX | | |
| | UAGUA | | mC*mC*mU*mU*mA*mG*mU*mA | | XXXX | | |
| WV- | AGUUUGGAGA | 242 | mA*mG*mU*mU*mU*mG*mG*mA*m | 994 | XXXXXXXXXX | 25-mer 2′-OMethyl | DMD |
| 2212 | UGGCAGUUUC | | G*mA*mU*mG*mG*mC*mA*mG*mU* | | XXXXXXXXXX | | |
| | CUUAG | | mU*mU*mC*mC*mU*mU*mA*mG | | XXXX | | |
| WV- | UAGUUUGGAG | 243 | mU*mA*mG*mU*mU*mU*mG*mG*m | 995 | XXXXXXXXXX | 25-mer 2′-OMethyl | DMD |
| 2213 | AUGGCAGUUU | | A*mG*mA*mU*mG*mG*mC*mA*mG* | | XXXXXXXXXX | | |
| | CCUUA | | mU*mU*mU*mC*mC*mU*mU*mA | | XXXX | | |
| WV- | CUAGUUUGGA | 244 | mC*mU*mA*mG*mU*mU*mU*mG*m | 996 | XXXXXXXXXX | 25-mer 2′-OMethyl | DMD |
| 2214 | GAUGGCAGUU | | G*mA*mG*mA*mU*mG*mG*mC*mA* | | XXXXXXXXXX | | |
| | UCCUU | | mG*mU*mU*mU*mC*mC*mU*mU | | XXXX | | |
| WV- | UCUAGUUUGG | 245 | mU*mC*mU*mA*mG*mU*mU*mU*m | 997 | XXXXXXXXXX | 25-mer 2′-OMethyl | DMD |
| 2215 | AGAUGGCAGU | | G*mG*mA*mG*mA*mU*mG*mG*mC* | | XXXXXXXXXX | | |
| | UUCCU | | mA*mG*mU*mU*mU*mC*mC*mU | | XXXX | | |
| WV- | UUCUAGUUUG | 246 | mU*mU*mC*mU*mA*mG*mU*mU*m | 998 | XXXXXXXXXX | 25-mer 2′-OMethyl | DMD |
| 2216 | GAGAUGGCAG | | U*mG*mG*mA*mG*mA*mU*mG*mG | | XXXXXXXXXX | | |
| | UUUCC | | *mC*mA*mG*mU*mU*mU*mC*mC | | XXXX | | |
| WV- | CAUUUCUAGU | 247 | mC*mA*mU*mU*mU*mC*mU*mA*m | 999 | XXXXXXXXXX | 25-mer 2′-OMethyl | DMD |
| 2217 | UUGGAGAUGG | | G*mU*mU*mU*mG*mG*mA*mG*mA | | XXXXXXXXXX | | |
| | CAGUU | | *mU*mG*mG*mC*mA*mG*mU*mU | | XXXX | | |
| WV- | GCAUUUCUAG | 248 | mG*mC*mA*mU*mU*mU*mC*mU*m | 1000 | XXXXXXXXXX | 25-mer 2′-OMethyl | DMD |
| 2218 | UUUGGAGAUG | | A*mG*mU*mU*mU*mG*mG*mA*mG | | XXXXXXXXXX | | |
| | GCAGU | | *mA*mU*mG*mG*mC*mA*mG*mU | | XXXX | | |
| WV- | AUGGCAUUUC | 249 | mA*mU*mG*mG*mC*mA*mU*mU*m | 1001 | XXXXXXXXXX | 25-mer 2′-OMethyl | DMD |
| 2219 | UAGUUUGGAG | | U*mC*mU*mA*mG*mU*mU*mU*mG* | | XXXXXXXXXX | | |
| | AUGGC | | mG*mA*mG*mA*mU*mG*mG*mC | | XXXX | | |
| WV- | GAAGAUGGCA | 250 | mG*mA*mA*mG*mA*mU*mG*mG*m | 1002 | XXXXXXXXXX | 25-mer 2′-OMethyl | DMD |
| 2220 | UUUCUAGUUU | | C*mA*mU*mU*mU*mC*mU*mA*mG* | | XXXXXXXXXX | | |
| | GGAGA | | mU*mU*mU*mG*mG*mA*mG*mA | | XXXX | | |
| WV- | AGGAAGAUGG | 251 | mA*mG*mG*mA*mA*mG*mA*mU*m | 1003 | XXXXXXXXXX | 25-mer 2′-OMethyl | DMD |
| 2221 | CAUUUCUAGU | | G*mG*mC*mA*mU*mU*mU*mC*mU* | | XXXXXXXXXX | | |
| | UUGGA | | mA*mG*mU*mU*mU*mG*mG*mA | | XXXX | | |
| WV- | AAGGAAGAUG | 252 | mA*mA*mG*mG*mA*mA*mG*mA*m | 1004 | XXXXXXXXXX | 25-mer 2′-OMethyl | DMD |
| 2222 | GCAUUUCUAG | | U*mG*mG*mC*mA*mU*mU*mU*mC* | | XXXXXXXXXX | | |
| | UUUGG | | mU*mA*mG*mU*mU*mU*mG*mG | | XXXX | | |
| WV- | CAAGGAAGAU | 253 | mC*mA*mA*mG*mG*mA*mA*mG*m | 1005 | XXXXXXXXXX | 25-mer 2′-OMethyl | DMD |
| 2223 | GGCAUUUCUA | | A*mU*mG*mG*mC*mA*mU*mU*mU* | | XXXXXXXXXX | | |
| | GUUUG | | mC*mU*mA*mG*mU*mU*mU*mG | | XXXX | | |
| WV- | CAUCAAGGAA | 254 | mC*mA*mU*mC*mA*mA*mG*mG*m | 1006 | XXXXXXXXXX | 25-mer 2′-OMethyl | DMD |
| 2224 | GAUGGCAUUU | | A*mA*mG*mA*mU*mG*mG*mC*mA* | | XXXXXXXXXX | | |
| | CUAGU | | mU*mU*mU*mC*mU*mA*mG*mU | | XXXX | | |
| WV- | ACAUCAAGGA | 255 | mA*mC*mA*mU*mC*mA*mA*mG*m | 1007 | XXXXXXXXXX | 25-mer 2′-OMethyl | DMD |
| 2225 | AGAUGGCAUU | | G*mA*mA*mG*mA*mU*mG*mG*mC* | | XXXXXXXXXX | | |
| | UCUAG | | mA*mU*mU*mU*mC*mU*mA*mG | | XXXX | | |
| WV- | AACAUCAAGG | 256 | mA*mA*mC*mA*mU*mC*mA*mA*m | 1008 | XXXXXXXXXX | 25-mer 2′-OMethyl | DMD |
| 2226 | AAGAUGGCAU | | G*mG*mA*mA*mG*mA*mU*mG*mG | | XXXXXXXXXX | | |
| | UUCUA | | *mC*mA*mU*mU*mU*mC*mU*mA | | XXXX | | |
| WV- | CAACAUCAAG | 257 | mC*mA*mA*mC*mA*mU*mC*mA*m | 1009 | XXXXXXXXXX | 25-mer 2′-OMethyl | DMD |
| 2227 | GAAGAUGGCA | | A*mG*mG*mA*mA*mG*mA*mU*mG | | XXXXXXXXXX | | |
| | UUUCU | | *mG*mC*mA*mU*mU*mU*mC*mU | | XXXX | | |
| WV- | CUCCAACAUCA | 258 | mC*mU*mC*mC*mA*mA*mC*mA*m | 1010 | XXXXXXXXXX | 25-mer 2′-OMethyl | DMD |
| 2228 | AGGAAGAUGG | | U*mC*mA*mA*mG*mG*mA*mA*mG* | | XXXXXXXXXX | | |
| | CAUU | | mA*mU*mG*mG*mC*mA*mU*mU | | XXXX | | |
| WV- | ACCUCCAACAU | 259 | mA*mC*mC*mU*mC*mC*mA*mA*m | 1011 | XXXXXXXXXX | 25-mer 2′-OMethyl | DMD |
| 2229 | CAAGGAAGAU | | C*mA*mU*mC*mA*mA*mG*mG*mA* | | XXXXXXXXXX | | |
| | GGCA | | mA*mG*mA*mU*mG*mG*mC*mA | | XXXX | | |
| WV- | GUACCUCCAAC | 260 | mG*mU*mA*mC*mC*mU*mC*mC*m | 1012 | XXXXXXXXXX | 25-mer 2′-OMethyl | DMD |
| 2230 | AUCAAGGAAG | | A*mA*mC*mA*mU*mC*mA*mA*mG* | | XXXXXXXXXX | | |
| | AUGG | | mG*mA*mA*mG*mA*mU*mG*mG | | XXXX | | |
| WV- | AGGUACCUCCA | 261 | mA*mG*mG*mU*mA*mC*mC*mU*m | 1013 | XXXXXXXXXX | 25-mer 2′-OMethyl | DMD |
| 2231 | ACAUCAAGGA | | C*mC*mA*mA*mC*mA*mU*mC*mA* | | XXXXXXXXXX | | |
| | AGAU | | mA*mG*mG*mA*mA*mG*mA*mU | | XXXX | | |
| WV- | AGAGCAGGUA | 262 | mA*mG*mA*mG*mC*mA*mG*mG*m | 1014 | XXXXXXXXXX | 25-mer 2′-OMethyl | DMD |
| 2232 | CCUCCAACAUC | | U*mA*mC*mC*mU*mC*mC*mA*mA* | | XXXXXXXXXX | | |
| | AAGG | | mC*mA*mU*mC*mA*mA*mG*mG | | XXXX | | |
| WV- | CAGAGCAGGU | 263 | mC*mA*mG*mA*mG*mC*mA*mG*m | 1015 | XXXXXXXXXX | 25-mer 2′-OMethyl | DMD |
| 2233 | ACCUCCAACAU | | G*mU*mA*mC*mC*mU*mC*mC*mA* | | XXXXXXXXXX | | |
| | CAAG | | mA*mC*mA*mU*mC*mA*mA*mG | | XXXX | | |
| WV- | CUGCCAGAGCA | 264 | mC*mU*mG*mC*mC*mA*mG*mA*m | 1016 | XXXXXXXXXX | 25-mer 2′-OMethyl | DMD |
| 2234 | GGUACCUCCAA | | G*mC*mA*mG*mG*mU*mA*mC*mC* | | XXXXXXXXXX | | |
| | CAU | | mU*mC*mC*mA*mA*mC*mA*mU | | XXXX | | |
| WV- | UCUGCCAGAGC | 265 | mU*mC*mU*mG*mC*mC*mA*mG*m | 1017 | XXXXXXXXXX | 25-mer 2′-OMethyl | DMD |
| 2235 | AGGUACCUCCA | | A*mG*mC*mA*mG*mG*mU*mA*mC* | | XXXXXXXXXX | | |
| | ACA | | mC*mU*mC*mC*mA*mA*mC*mA | | XXXX | | |
| WV- | AUCUGCCAGA | 266 | mA*mU*mC*mU*mG*mC*mC*mA*m | 1018 | XXXXXXXXXX | 25-mer 2′-OMethyl | DMD |
| 2236 | GCAGGUACCUC | | G*mA*mG*mC*mA*mG*mG*mU*mA* | | XXXXXXXXXX | | |
| | CAAC | | mC*mC*mU*mC*mC*mA*mA*mC | | XXXX | | |
| WV- | AAUCUGCCAG | 267 | mA*mA*mU*mC*mU*mG*mC*mC*m | 1019 | XXXXXXXXXX | 25-mer 2′-OMethyl | DMD |
| 2237 | AGCAGGUACC | | A*mG*mA*mG*mC*mA*mG*mG*mU* | | XXXXXXXXXX | | |
| | UCCAA | | mA*mC*mC*mU*mC*mC*mA*mA | | XXXX | | |
| WV- | AAAUCUGCCA | 268 | mA*mA*mA*mU*mC*mU*mG*mC*m | 1020 | XXXXXXXXXX | 25-mer 2′-OMethyl | DMD |
| 2238 | GAGCAGGUAC | | C*mA*mG*mA*mG*mC*mA*mG*mG* | | XXXXXXXXXX | | |
| | CUCCA | | mU*mA*mC*mC*mU*mC*mC*mA | | XXXX | | |
| WV- | GAAAUCUGCC | 269 | mG*mA*mA*mA*mU*mC*mU*mG*m | 1021 | XXXXXXXXXX | 25-mer 2′-OMethyl | DMD |
| 2239 | AGAGCAGGUA | | C*mC*mA*mG*mA*mG*mC*mA*mG* | | XXXXXXXXXX | | |
| | CCUCC | | mG*mU*mA*mC*mC*mU*mC*mC | | XXXX | | |
| WV- | UGAAAUCUGC | 270 | mU*mG*mA*mA*mA*mU*mC*mU*m | 1022 | XXXXXXXXXX | 25-mer 2′-OMethyl | DMD |
| 2240 | CAGAGCAGGU | | G*mC*mC*mA*mG*mA*mG*mC*mA* | | XXXXXXXXXX | | |
| | ACCUC | | mG*mG*mU*mA*mC*mC*mU*mC | | XXXX | | |
| WV- | UUGAAAUCUG | 271 | mU*mU*mG*mA*mA*mA*mU*mC*m | 1023 | XXXXXXXXXX | 25-mer 2′-OMethyl | DMD |
| 2241 | CCAGAGCAGG | | U*mG*mC*mC*mA*mG*mA*mG*mC* | | XXXXXXXXXX | | |
| | UACCU | | mA*mG*mG*mU*mA*mC*mC*mU | | XXXX | | |
| WV- | CCCGGUUGAA | 272 | mC*mC*mC*mG*mG*mU*mU*mG*m | 1024 | XXXXXXXXXX | 25-mer 2′-OMethyl | DMD |
| 2242 | AUCUGCCAGA | | A*mA*mA*mU*mC*mU*mG*mC*mC* | | XXXXXXXXXX | | |
| | GCAGG | | mA*mG*mA*mG*mC*mA*mG*mG | | XXXX | | |
| WV- | CCAAGCCCGGU | 273 | mC*mC*mA*mA*mG*mC*mC*mC*m | 1025 | XXXXXXXXXX | 25-mer 2′-OMethyl | DMD |
| 2243 | UGAAAUCUGC | | G*mG*mU*mU*mG*mA*mA*mA*mU | | XXXXXXXXXX | | |
| | CAGA | | *mC*mU*mG*mC*mC*mA*mG*mA | | XXXX | | |
| WV- | UCCAAGCCCGG | 274 | mU*mC*mC*mA*mA*mG*mC*mC*m | 1026 | XXXXXXXXXX | 25-mer 2′-OMethyl | DMD |
| 2244 | UUGAAAUCUG | | C*mG*mG*mU*mU*mG*mA*mA*mA* | | XXXXXXXXXX | | |
| | CCAG | | mU*mC*mU*mG*mC*mC*mA*mG | | XXXX | | |
| WV- | GUCCAAGCCCG | 275 | mG*mU*mC*mC*mA*mA*mG*mC*m | 1027 | XXXXXXXXXX | 25-mer 2′-OMethyl | DMD |
| 2245 | GUUGAAAUCU | | C*mC*mG*mG*mU*mU*mG*mA*mA* | | XXXXXXXXXX | | |
| | GCCA | | mA*mU*mC*mU*mG*mC*mC*mA | | XXXX | | |
| WV- | UCUGUCCAAGC | 276 | mU*mC*mU*mG*mU*mC*mC*mA*m | 1028 | XXXXXXXXXX | 25-mer 2′-OMethyl | DMD |
| 2246 | CCGGUUGAAA | | A*mG*mC*mC*mC*mG*mG*mU*mU* | | XXXXXXXXXX | | |
| | UCUG | | mG*mA*mA*mA*mU*mC*mU*mG | | XXXX | | |
| WV- | UUCUGUCCAA | 277 | mU*mU*mC*mU*mG*mU*mC*mC*m | 1029 | XXXXXXXXXX | 25-mer 2′-OMethyl | DMD |
| 2247 | GCCCGGUUGA | | A*mA*mG*mC*mC*mC*mG*mG*mU* | | XXXXXXXXXX | | |
| | AAUCU | | mU*mG*mA*mA*mA*mU*mC*mU | | XXXX | | |
| WV- | GUUCUGUCCA | 278 | mG*mU*mU*mC*mU*mG*mU*mC*m | 1030 | XXXXXXXXXX | 25-mer 2′-OMethyl | DMD |
| 2248 | AGCCCGGUUG | | C*mA*mA*mG*mC*mC*mC*mG*mG* | | XXXXXXXXXX | | |
| | AAAUC | | mU*mU*mG*mA*mA*mA*mU*mC | | XXXX | | |
| WV- | AGUUCUGUCC | 279 | mA*mG*mU*mU*mC*mU*mG*mU*m | 1031 | XXXXXXXXXX | 25-mer 2′-OMethyl | DMD |
| 2249 | AAGCCCGGUU | | C*mC*mA*mA*mG*mC*mC*mC*mG* | | XXXXXXXXXX | | |
| | GAAAU | | mG*mU*mU*mG*mA*mA*mA*mU | | XXXX | | |
| WV- | AAGUUCUGUC | 280 | mA*mA*mG*mU*mU*mC*mU*mG*m | 1032 | XXXXXXXXXX | 25-mer 2′-OMethyl | DMD |
| 2250 | CAAGCCCGGUU | | U*mC*mC*mA*mA*mG*mC*mC*mC* | | XXXXXXXXXX | | |
| | GAAA | | mG*mG*mU*mU*mG*mA*mA*mA | | XXXX | | |
| WV- | UAAGUUCUGU | 281 | mU*mA*mA*mG*mU*mU*mC*mU*m | 1033 | XXXXXXXXXX | 25-mer 2′-OMethyl | DMD |
| 2251 | CCAAGCCCGGU | | G*mU*mC*mC*mA*mA*mG*mC*mC* | | XXXXXXXXXX | | |
| | UGAA | | mC*mG*mG*mU*mU*mG*mA*mA | | XXXX | | |
| WV- | GUAAGUUCUG | 282 | mG*mU*mA*mA*mG*mU*mU*mC*m | 1034 | XXXXXXXXXX | 25-mer 2′-OMethyl | DMD |
| 2252 | UCCAAGCCCGG | | U*mG*mU*mC*mC*mA*mA*mG*mC* | | XXXXXXXXXX | | |
| | UUGA | | mC*mC*mG*mG*mU*mU*mG*mA | | XXXX | | |
| WV- | GGUAAGUUCU | 283 | mG*mG*mU*mA*mA*mG*mU*mU*m | 1035 | XXXXXXXXXX | 25-mer 2′-OMethyl | DMD |
| 2253 | GUCCAAGCCCG | | C*mU*mG*mU*mC*mC*mA*mA*mG* | | XXXXXXXXXX | | |
| | GUUG | | mC*mC*mC*mG*mG*mU*mU*mG | | XXXX | | |
| WV- | CGGUAAGUUC | 284 | mC*mG*mG*mU*mA*mA*mG*mU*m | 1036 | XXXXXXXXXX | 25-mer 2′-OMethyl | DMD |
| 2254 | UGUCCAAGCCC | | U*mC*mU*mG*mU*mC*mC*mA*mA* | | XXXXXXXXXX | | |
| | GGUU | | mG*mC*mC*mC*mG*mG*mU*mU | | XXXX | | |
| WV- | UCGGUAAGUU | 285 | mU*mC*mG*mG*mU*mA*mA*mG*m | 1037 | XXXXXXXXXX | 25-mer 2′-OMethyl | DMD |
| 2255 | CUGUCCAAGCC | | U*mU*mC*mU*mG*mU*mC*mC*mA* | | XXXXXXXXXX | | |
| | CGGU | | mA*mG*mC*mC*mC*mG*mG*mU | | XXXX | | |
| WV- | GUCGGUAAGU | 286 | mG*mU*mC*mG*mG*mU*mA*mA*m | 1038 | XXXXXXXXXX | 25-mer 2′-OMethyl | DMD |
| 2256 | UCUGUCCAAGC | | G*mU*mU*mC*mU*mG*mU*mC*mC* | | XXXXXXXXXX | | |
| | CCGG | | mA*mA*mG*mC*mC*mC*mG*mG | | XXXX | | |
| WV- | AGUCGGUAAG | 287 | mA*mG*mU*mC*mG*mG*mU*mA*m | 1039 | XXXXXXXXXX | 25-mer 2′-OMethyl | DMD |
| 2257 | UUCUGUCCAA | | A*mG*mU*mU*mC*mU*mG*mU*mC* | | XXXXXXXXXX | | |
| | GCCCG | | mC*mA*mA*mG*mC*mC*mC*mG | | XXXX | | |
| WV- | CAGUCGGUAA | 288 | mC*mA*mG*mU*mC*mG*mG*mU*m | 1040 | XXXXXXXXXX | 25-mer 2′-OMethyl | DMD |
| 2258 | GUUCUGUCCA | | A*mA*mG*mU*mU*mC*mU*mG*mU* | | XXXXXXXXXX | | |
| | AGCCC | | mC*mC*mA*mA*mG*mC*mC*mC | | XXXX | | |
| WV- | AAAGCCAGUC | 289 | mA*mA*mA*mG*mC*mC*mA*mG*m | 1041 | XXXXXXXXXX | 25-mer 2′-OMethyl | DMD |
| 2259 | GGUAAGUUCU | | U*mC*mG*mG*mU*mA*mA*mG*mU* | | XXXXXXXXXX | | |
| | GUCCA | | mU*mC*mU*mG*mU*mC*mC*mA | | XXXX | | |
| WV- | GAAAGCCAGU | 290 | mG*mA*mA*mA*mG*mC*mC*mA*m | 1042 | XXXXXXXXXX | 25-mer 2′-OMethyl | DMD |
| 2260 | CGGUAAGUUC | | G*mU*mC*mG*mG*mU*mA*mA*mG* | | XXXXXXXXXX | | |
| | UGUCC | | mU*mU*mC*mU*mG*mU*mC*mC | | XXXX | | |
| WV- | GUCACCCACCA | 291 | mG*mU*mC*mA*mC*mC*mC*mA*m | 1043 | XXXXXXXXXX | 25-mer 2′-OMethyl | DMD |
| 2261 | UCACCCUCUGU | | C*mC*mA*mU*mC*mA*mC*mC*mC* | | XXXXXXXXXX | | |
| | GAU | | mU*mC*mU*mG*mU*mG*mA*mU | | XXXX | | |
| WV- | GGUCACCCACC | 292 | mG*mG*mU*mC*mA*mC*mC*mC*m | 1044 | XXXXXXXXXX | 25-mer 2′-OMethyl | DMD |
| 2262 | AUCACCCUCUG | | A*mC*mC*mA*mU*mC*mA*mC*mC* | | XXXXXXXXXX | | |
| | UGA | | mC*mU*mC*mU*mG*mU*mG*mA | | XXXX | | |
| WV- | AAGGUCACCCA | 293 | mA*mA*mG*mG*mU*mC*mA*mC*m | 1045 | XXXXXXXXXX | 25-mer 2′-OMethyl | DMD |
| 2263 | CCAUCACCCUC | | C*mC*mA*mC*mC*mA*mU*mC*mA* | | XXXXXXXXXX | | |
| | UGU | | mC*mC*mC*mU*mC*mU*mG*mU | | XXXX | | |
| WV- | CAAGGUCACCC | 294 | mC*mA*mA*mG*mG*mU*mC*mA*m | 1046 | XXXXXXXXXX | 25-mer 2′-OMethyl | DMD |
| 2264 | ACCAUCACCCU | | C*mC*mC*mA*mC*mC*mA*mU*mC* | | XXXXXXXXXX | | |
| | CUG | | mA*mC*mC*mC*mU*mC*mU*mG | | XXXX | | |
| WV- | UCAAGGUCACC | 295 | mU*mC*mA*mA*mG*mG*mU*mC*m | 1047 | XXXXXXXXXX | 25-mer 2′-OMethyl | DMD |
| 2265 | CACCAUCACCC | | A*mC*mC*mC*mA*mC*mC*mA*mU* | | XXXXXXXXXX | | |
| | UCU | | mC*mA*mC*mC*mC*mU*mC*mU | | XXXX | | |
| WV- | CUCAAGGUCAC | 296 | mC*mU*mC*mA*mA*mG*mG*mU*m | 1048 | XXXXXXXXXX | 25-mer 2′-OMethyl | DMD |
| 2266 | CCACCAUCACC | | C*mA*mC*mC*mC*mA*mC*mC*mA* | | XXXXXXXXXX | | |
| | CUC | | mU*mC*mA*mC*mC*mC*mU*mC | | XXXX | | |
| WV- | CUUGAUCAAG | 297 | mC*mU*mU*mG*mA*mU*mC*mA*m | 1049 | XXXXXXXXXX | 25-mer 2′-OMethyl | DMD |
| 2267 | CAGAGAAAGC | | A*mG*mC*mA*mG*mA*mG*mA*mA* | | XXXXXXXXXX | | |
| | CAGUC | | mA*mG*mC*mC*mA*mG*mU*mC | | XXXX | | |
| WV- | AUAACUUGAU | 298 | mA*mU*mA*mA*mC*mU*mU*mG*m | 1050 | XXXXXXXXXX | 25-mer 2′-OMethyl | DMD |
| 2268 | CAAGCAGAGA | | A*mU*mC*mA*mA*mG*mC*mA*mG* | | XXXXXXXXXX | | |
| | AAGCC | | mA*mG*mA*mA*mA*mG*mC*mC | | XXXX | | |
| WV- | AGUAACAGUC | 299 | mA*mG*mU*mA*mA*mC*mA*mG*m | 1051 | XXXXXXXXXX | 20-mer 2′-OMethyl | DMD |
| 2273 | UGAGUAGGAG | | U*mC*mU*mG*mA*mG*mU*mA*mG* | | XXXXXXXXX | | |
| | | | mG*mA*mG | | | | |
| WV- | GAGUAACAGU | 300 | mG*mA*mG*mU*mA*mA*mC*mA*m | 1052 | XXXXXXXXXX | 20-mer 2′-OMethyl | DMD |
| 2274 | CUGAGUAGGA | | G*mU*mC*mU*mG*mA*mG*mU*mA* | | XXXXXXXXX | | |
| | | | mG*mG*mA | | | | |
| WV- | AGAGUAACAG | 301 | mA*mG*mA*mG*mU*mA*mA*mC*m | 1053 | XXXXXXXXXX | 20-mer 2′-OMethyl | DMD |
| 2275 | UCUGAGUAGG | | A*mG*mU*mC*mU*mG*mA*mG*mU* | | XXXXXXXXX | | |
| | | | mA*mG*mG | | | | |
| WV- | CAGAGUAACA | 302 | mC*mA*mG*mA*mG*mU*mA*mA*m | 1054 | XXXXXXXXXX | 20-mer 2′-OMethyl | DMD |
| 2276 | GUCUGAGUAG | | C*mA*mG*mU*mC*mU*mG*mA*mG* | | XXXXXXXXX | | |
| | | | mU*mA*mG | | | | |
| WV- | GUCACCAGAG | 303 | mG*mU*mC*mA*mC*mC*mA*mG*m | 1055 | XXXXXXXXXX | 20-mer 2′-OMethyl | DMD |
| 2277 | UAACAGUCUG | | A*mG*mU*mA*mA*mC*mA*mG*mU* | | XXXXXXXXX | | |
| | | | mC*mU*mG | | | | |
| WV- | UGUCACCAGA | 304 | mU*mG*mU*mC*mA*mC*mC*mA*m | 1056 | XXXXXXXXXX | 20-mer 2′-OMethyl | DMD |
| 2278 | GUAACAGUCU | | G*mA*mG*mU*mA*mA*mC*mA*mG* | | XXXXXXXXX | | |
| | | | mU*mC*mU | | | | |
| WV- | GUGUCACCAG | 305 | mG*mU*mG*mU*mC*mA*mC*mC*m | 1057 | XXXXXXXXXX | 20-mer 2′-OMethyl | DMD |
| 2279 | AGUAACAGUC | | A*mG*mA*mG*mU*mA*mA*mC*mA* | | XXXXXXXXX | | |
| | | | mG*mU*mC | | | | |
| WV- | UGUGUCACCA | 306 | mU*mG*mU*mG*mU*mC*mA*mC*m | 1058 | XXXXXXXXXX | 20-mer 2′-OMethyl | DMD |
| 2280 | GAGUAACAGU | | C*mA*mG*mA*mG*mU*mA*mA*mC* | | XXXXXXXXX | | |
| | | | mA*mG*mU | | | | |
| WV- | UUGUGUCACC | 307 | mU*mU*mG*mU*mG*mU*mC*mA*m | 1059 | XXXXXXXXXX | 20-mer 2′-OMethyl | DMD |
| 2281 | AGAGUAACAG | | C*mC*mA*mG*mA*mG*mU*mA*mA* | | XXXXXXXXX | | |
| | | | mC*mA*mG | | | | |
| WV- | GGUUGUGUCA | 308 | mG*mG*mU*mU*mG*mU*mG*mU*m | 1060 | XXXXXXXXXX | 20-mer 2′-OMethyl | DMD |
| 2282 | CCAGAGUAAC | | C*mA*mC*mC*mA*mG*mA*mG*mU* | | XXXXXXXXX | | |
| | | | mA*mA*mC | | | | |
| WV- | AGGUUGUGUC | 309 | mA*mG*mG*mU*mU*mG*mU*mG*m | 1061 | XXXXXXXXXX | 20-mer 2′-OMethyl | DMD |
| 2283 | ACCAGAGUAA | | U*mC*mA*mC*mC*mA*mG*mA*mG* | | XXXXXXXXX | | |
| | | | mU*mA*mA | | | | |
| WV- | CAGGUUGUGU | 310 | mC*mA*mG*mG*mU*mU*mG*mU*m | 1062 | XXXXXXXXXX | 20-mer 2′-OMethyl | DMD |
| 2284 | CACCAGAGUA | | G*mU*mC*mA*mC*mC*mA*mG*mA* | | XXXXXXXXX | | |
| | | | mG*mU*mA | | | | |
| WV- | ACAGGUUGUG | 311 | mA*mC*mA*mG*mG*mU*mU*mG*m | 1063 | XXXXXXXXXX | 20-mer 2′-OMethyl | DMD |
| 2285 | UCACCAGAGU | | U*mG*mU*mC*mA*mC*mC*mA*mG* | | XXXXXXXXX | | |
| | | | mA*mG*mU | | | | |
| WV- | CACAGGUUGU | 312 | mC*mA*mC*mA*mG*mG*mU*mU*m | 1064 | XXXXXXXXXX | 20-mer 2′-OMethyl | DMD |
| 2286 | GUCACCAGAG | | G*mU*mG*mU*mC*mA*mC*mC*mA* | | XXXXXXXXX | | |
| | | | mG*mA*mG | | | | |
| WV- | CCACAGGUUG | 313 | mC*mC*mA*mC*mA*mG*mG*mU*m | 1065 | XXXXXXXXXX | 20-mer 2′-OMethyl | DMD |
| 2287 | UGUCACCAGA | | U*mG*mU*mG*mU*mC*mA*mC*mC* | | XXXXXXXXX | | |
| | | | mA*mG*mA | | | | |
| WV- | ACCACAGGUU | 314 | mA*mC*mC*mA*mC*mA*mG*mG*m | 1066 | XXXXXXXXXX | 20-mer 2′-OMethyl | DMD |
| 2288 | GUGUCACCAG | | U*mU*mG*mU*mG*mU*mC*mA*mC* | | XXXXXXXXX | | |
| | | | mC*mA*mG | | | | |
| WV- | AACCACAGGU | 315 | mA*mA*mC*mC*mA*mC*mA*mG*m | 1067 | XXXXXXXXXX | 20-mer 2′-OMethyl | DMD |
| 2289 | UGUGUCACCA | | G*mU*mU*mG*mU*mG*mU*mC*mA* | | XXXXXXXXX | | |
| | | | mC*mC*mA | | | | |
| WV- | UAACCACAGG | 316 | mU*mA*mA*mC*mC*mA*mC*mA*m | 1068 | XXXXXXXXXX | 20-mer 2′-OMethyl | DMD |
| 2290 | UUGUGUCACC | | G*mG*mU*mU*mG*mU*mG*mU*mC* | | XXXXXXXXX | | |
| | | | mA*mC*mC | | | | |
| WV- | GUAACCACAG | 317 | mG*mU*mA*mA*mC*mC*mA*mC*m | 1069 | XXXXXXXXXX | 20-mer 2′-OMethyl | DMD |
| 2291 | GUUGUGUCAC | | A*mG*mG*mU*mU*mG*mU*mG*mU | | XXXXXXXXX | | |
| | | | *mC*mA*mC | | | | |
| WV- | AGUAACCACA | 318 | mA*mG*mU*mA*mA*mC*mC*mA*m | 1070 | XXXXXXXXXX | 20-mer 2′-OMethyl | DMD |
| 2292 | GGUUGUGUCA | | C*mA*mG*mG*mU*mU*mG*mU*mG* | | XXXXXXXXX | | |
| | | | mU*mC*mA | | | | |
| WV- | CUUAGUAACC | 319 | mC*mU*mU*mA*mG*mU*mA*mA*m | 1071 | XXXXXXXXXX | 20-mer 2′-OMethyl | DMD |
| 2293 | ACAGGUUGUG | | C*mC*mA*mC*mA*mG*mG*mU*mU* | | XXXXXXXXX | | |
| | | | mG*mU*mG | | | | |
| WV- | CCUUAGUAACC | 320 | mC*mC*mU*mU*mA*mG*mU*mA*m | 1072 | XXXXXXXXXX | 20-mer 2′-OMethyl | DMD |
| 2294 | ACAGGUUGU | | A*mC*mC*mA*mC*mA*mG*mG*mU* | | XXXXXXXXX | | |
| | | | mU*mG*mU | | | | |
| WV- | UCCUUAGUAA | 321 | mU*mC*mC*mU*mU*mA*mG*mU*m | 1073 | XXXXXXXXXX | 20-mer 2′-OMethyl | DMD |
| 2295 | CCACAGGUUG | | A*mA*mC*mC*mA*mC*mA*mG*mG* | | XXXXXXXXX | | |
| | | | mU*mU*mG | | | | |
| WV- | UUCCUUAGUA | 322 | mU*mU*mC*mC*mU*mU*mA*mG*m | 1074 | XXXXXXXXXX | 20-mer 2′-OMethyl | DMD |
| 2296 | ACCACAGGUU | | U*mA*mA*mC*mC*mA*mC*mA*mG* | | XXXXXXXXX | | |
| | | | mG*mU*mU | | | | |
| WV- | UUUCCUUAGU | 323 | mU*mU*mU*mC*mC*mU*mU*mA*m | 1075 | XXXXXXXXXX | 20-mer 2′-OMethyl | DMD |
| 2297 | AACCACAGGU | | G*mU*mA*mA*mC*mC*mA*mC*mA* | | XXXXXXXXX | | |
| | | | mG*mG*mU | | | | |
| WV- | GUUUCCUUAG | 324 | mG*mU*mU*mU*mC*mC*mU*mU*m | 1076 | XXXXXXXXXX | 20-mer 2′-OMethyl | DMD |
| 2298 | UAACCACAGG | | A*mG*mU*mA*mA*mC*mC*mA*mC* | | XXXXXXXXX | | |
| | | | mA*mG*mG | | | | |
| WV- | AGUUUCCUUA | 325 | mA*mG*mU*mU*mU*mC*mC*mU*m | 1077 | XXXXXXXXXX | 20-mer 2′-OMethyl | DMD |
| 2299 | GUAACCACAG | | U*mA*mG*mU*mA*mA*mC*mC*mA* | | XXXXXXXXX | | |
| | | | mC*mA*mG | | | | |
| WV- | GCAGUUUCCU | 326 | mG*mC*mA*mG*mU*mU*mU*mC*m | 1078 | XXXXXXXXXX | 20-mer 2′-OMethyl | DMD |
| 2300 | UAGUAACCAC | | C*mU*mU*mA*mG*mU*mA*mA*mC* | | XXXXXXXXX | | |
| | | | mC*mA*mC | | | | |
| WV- | GGCAGUUUCC | 327 | mG*mG*mC*mA*mG*mU*mU*mU*m | 1079 | XXXXXXXXXX | 20-mer 2′-OMethyl | DMD |
| 2301 | UUAGUAACCA | | C*mC*mU*mU*mA*mG*mU*mA*mA* | | XXXXXXXXX | | |
| | | | mC*mC*mA | | | | |
| WV- | UGGCAGUUUC | 328 | mU*mG*mG*mC*mA*mG*mU*mU*m | 1080 | XXXXXXXXXX | 20-mer 2′-OMethyl | DMD |
| 2302 | CUUAGUAACC | | U*mC*mC*mU*mU*mA*mG*mU*mA* | | XXXXXXXXX | | |
| | | | mA*mC*mC | | | | |
| WV- | AUGGCAGUUU | 329 | mA*mU*mG*mG*mC*mA*mG*mU*m | 1081 | XXXXXXXXXX | 20-mer 2′-OMethyl | DMD |
| 2303 | CCUUAGUAAC | | U*mU*mC*mC*mU*mU*mA*mG*mU* | | XXXXXXXXX | | |
| | | | mA*mA*mC | | | | |
| WV- | GAUGGCAGUU | 330 | mG*mA*mU*mG*mG*mC*mA*mG*m | 1082 | XXXXXXXXXX | 20-mer 2′-OMethyl | DMD |
| 2304 | UCCUUAGUAA | | U*mU*mU*mC*mC*mU*mU*mA*mG* | | XXXXXXXXX | | |
| | | | mU*mA*mA | | | | |
| WV- | AGAUGGCAGU | 331 | mA*mG*mA*mU*mG*mG*mC*mA*m | 1083 | XXXXXXXXXX | 20-mer 2′-OMethyl | DMD |
| 2305 | UUCCUUAGUA | | G*mU*mU*mU*mC*mC*mU*mU*mA* | | XXXXXXXXX | | |
| | | | mG*mU*mA | | | | |
| WV- | GGAGAUGGCA | 332 | mG*mG*mA*mG*mA*mU*mG*mG*m | 1084 | XXXXXXXXXX | 20-mer 2′-OMethyl | DMD |
| 2306 | GUUUCCUUAG | | C*mA*mG*mU*mU*mU*mC*mC*mU* | | XXXXXXXXX | | |
| | | | mU*mA*mG | | | | |
| WV- | UGGAGAUGGC | 333 | mU*mG*mG*mA*mG*mA*mU*mG*m | 1085 | XXXXXXXXXX | 20-mer 2′-OMethyl | DMD |
| 2307 | AGUUUCCUUA | | G*mC*mA*mG*mU*mU*mU*mC*mC* | | XXXXXXXXX | | |
| | | | mU*mU*mA | | | | |
| WV- | UUGGAGAUGG | 334 | mU*mU*mG*mG*mA*mG*mA*mU*m | 1086 | XXXXXXXXXX | 20-mer 2′-OMethyl | DMD |
| 2308 | CAGUUUCCUU | | G*mG*mC*mA*mG*mU*mU*mU*mC* | | XXXXXXXXX | | |
| | | | mC*mU*mU | | | | |
| WV- | UUUGGAGAUG | 335 | mU*mU*mU*mG*mG*mA*mG*mA*m | 1087 | XXXXXXXXXX | 20-mer 2′-OMethyl | DMD |
| 2309 | GCAGUUUCCU | | U*mG*mG*mC*mA*mG*mU*mU*mU* | | XXXXXXXXX | | |
| | | | mC*mC*mU | | | | |
| WV- | GUUUGGAGAU | 336 | mG*mU*mU*mU*mG*mG*mA*mG*m | 1088 | XXXXXXXXXX | 20-mer 2′-OMethyl | DMD |
| 2310 | GGCAGUUUCC | | A*mU*mG*mG*mC*mA*mG*mU*mU* | | XXXXXXXXX | | |
| | | | mU*mC*mC | | | | |
| WV- | CUAGUUUGGA | 337 | mC*mU*mA*mG*mU*mU*mU*mG*m | 1089 | XXXXXXXXXX | 20-mer 2′-OMethyl | DMD |
| 2311 | GAUGGCAGUU | | G*mA*mG*mA*mU*mG*mG*mC*mA* | | XXXXXXXXX | | |
| | | | mG*mU*mU | | | | |
| WV- | UCUAGUUUGG | 338 | mU*mC*mU*mA*mG*mU*mU*mU*m | 1090 | XXXXXXXXXX | 20-mer 2′-OMethyl | DMD |
| 2312 | AGAUGGCAGU | | G*mG*mA*mG*mA*mU*mG*mG*mC* | | XXXXXXXXX | | |
| | | | mA*mG*mU | | | | |
| WV- | AUUUCUAGUU | 339 | mA*mU*mU*mU*mC*mU*mA*mG*m | 1091 | XXXXXXXXXX | 20-mer 2′-OMethyl | DMD |
| 2313 | UGGAGAUGGC | | U*mU*mU*mG*mG*mA*mG*mA*mU | | XXXXXXXXX | | |
| | | | *mG*mG*mC | | | | |
| WV- | UGGCAUUUCU | 340 | mU*mG*mG*mC*mA*mU*mU*mU*m | 1092 | XXXXXXXXXX | 20-mer 2′-OMethyl | DMD |
| 2314 | AGUUUGGAGA | | C*mU*mA*mG*mU*mU*mU*mG*mG* | | XXXXXXXXX | | |
| | | | mA*mG*mA | | | | |
| WV- | GAUGGCAUUU | 341 | mG*mA*mU*mG*mG*mC*mA*mU*m | 1093 | XXXXXXXXXX | 20-mer 2′-OMethyl | DMD |
| 2315 | CUAGUUUGGA | | U*mU*mC*mU*mA*mG*mU*mU*mU* | | XXXXXXXXX | | |
| | | | mG*mG*mA | | | | |
| WV- | AGAUGGCAUU | 342 | mA*mG*mA*mU*mG*mG*mC*mA*m | 1094 | XXXXXXXXXX | 20-mer 2′-OMethyl | DMD |
| 2316 | UCUAGUUUGG | | U*mU*mU*mC*mU*mA*mG*mU*mU* | | XXXXXXXXX | | |
| | | | mU*mG*mG | | | | |
| WV- | AAGAUGGCAU | 343 | mA*mA*mG*mA*mU*mG*mG*mC*m | 1095 | XXXXXXXXXX | 20-mer 2′-OMethyl | DMD |
| 2317 | UUCUAGUUUG | | A*mU*mU*mU*mC*mU*mA*mG*mU* | | XXXXXXXXX | | |
| | | | mU*mU*mG | | | | |
| WV- | AGGAAGAUGG | 344 | mA*mG*mG*mA*mA*mG*mA*mU*m | 1096 | XXXXXXXXXX | 20-mer 2′-OMethyl | DMD |
| 2318 | CAUUUCUAGU | | G*mG*mC*mA*mU*mU*mU*mC*mU* | | XXXXXXXXX | | |
| | | | mA*mG*mU | | | | |
| WV- | AAGGAAGAUG | 345 | mA*mA*mG*mG*mA*mA*mG*mA*m | 1097 | XXXXXXXXXX | 20-mer 2′-OMethyl | DMD |
| 2319 | GCAUUUCUAG | | U*mG*mG*mC*mA*mU*mU*mU*mC* | | XXXXXXXXX | | |
| | | | mU*mA*mG | | | | |
| WV- | CAAGGAAGAU | 346 | mC*mA*mA*mG*mG*mA*mA*mG*m | 1098 | XXXXXXXXXX | 20-mer 2′-OMethyl | DMD |
| 2320 | GGCAUUUCUA | | A*mU*mG*mG*mC*mA*mU*mU*mU* | | XXXXXXXXX | | |
| | | | mC*mU*mA | | | | |
| WV- | UCAAGGAAGA | 347 | mU*mC*mA*mA*mG*mG*mA*mA*m | 1099 | XXXXXXXXXX | 20-mer 2′-OMethyl | DMD |
| 2321 | UGGCAUUUCU | | G*mA*mU*mG*mG*mC*mA*mU*mU* | | XXXXXXXXX | | |
| | | | mU*mC*mU | | | | |
| WV- | ACAUCAAGGA | 348 | mA*mC*mA*mU*mC*mA*mA*mG*m | 1100 | XXXXXXXXXX | 20-mer 2′-OMethyl | DMD |
| 2322 | AGAUGGCAUU | | G*mA*mA*mG*mA*mU*mG*mG*mC* | | XXXXXXXXX | | |
| | | | mA*mU*mU | | | | |
| WV- | CAACAUCAAG | 349 | mC*mA*mA*mC*mA*mU*mC*mA*m | 1101 | XXXXXXXXXX | 20-mer 2′-OMethyl | DMD |
| 2323 | GAAGAUGGCA | | A*mG*mG*mA*mA*mG*mA*mU*mG | | XXXXXXXXX | | |
| | | | *mG*mC*mA | | | | |
| WV- | UCCAACAUCAA | 350 | mU*mC*mC*mA*mA*mC*mA*mU*m | 1102 | XXXXXXXXXX | 20-mer 2′-OMethyl | DMD |
| 2324 | GGAAGAUGG | | C*mA*mA*mG*mG*mA*mA*mG*mA* | | XXXXXXXXX | | |
| | | | mU*mG*mG | | | | |
| WV- | CCUCCAACAUC | 351 | mC*mC*mU*mC*mC*mA*mA*mC*m | 1103 | XXXXXXXXXX | 20-mer 2′-OMethyl | DMD |
| 2325 | AAGGAAGAU | | A*mU*mC*mA*mA*mG*mG*mA*mA* | | XXXXXXXXX | | |
| | | | mG*mA*mU | | | | |
| WV- | AGGUACCUCCA | 352 | mA*mG*mG*mU*mA*mC*mC*mU*m | 1104 | XXXXXXXXXX | 20-mer 2′-OMethyl | DMD |
| 2326 | ACAUCAAGG | | C*mC*mA*mA*mC*mA*mU*mC*mA* | | XXXXXXXXX | | |
| | | | mA*mG*mG | | | | |
| WV- | CAGGUACCUCC | 353 | mC*mA*mG*mG*mU*mA*mC*mC*m | 1105 | XXXXXXXXXX | 20-mer 2′-OMethyl | DMD |
| 2327 | AACAUCAAG | | U*mC*mC*mA*mA*mC*mA*mU*mC* | | XXXXXXXXX | | |
| | | | mA*mA*mG | | | | |
| WV- | AGAGCAGGUA | 354 | mA*mG*mA*mG*mC*mA*mG*mG*m | 1106 | XXXXXXXXXX | 20-mer 2′-OMethyl | DMD |
| 2328 | CCUCCAACAU | | U*mA*mC*mC*mU*mC*mC*mA*mA* | | XXXXXXXXX | | |
| | | | mC*mA*mU | | | | |
| WV- | CAGAGCAGGU | 355 | mC*mA*mG*mA*mG*mC*mA*mG*m | 1107 | XXXXXXXXXX | 20-mer 2′-OMethyl | DMD |
| 2329 | ACCUCCAACA | | G*mU*mA*mC*mC*mU*mC*mC*mA* | | XXXXXXXXX | | |
| | | | mA*mC*mA | | | | |
| WV- | CCAGAGCAGG | 356 | mC*mC*mA*mG*mA*mG*mC*mA*m | 1108 | XXXXXXXXXX | 20-mer 2′-OMethyl | DMD |
| 2330 | UACCUCCAAC | | G*mG*mU*mA*mC*mC*mU*mC*mC* | | XXXXXXXXX | | |
| | | | mA*mA*mC | | | | |
| WV- | GCCAGAGCAG | 357 | mG*mC*mC*mA*mG*mA*mG*mC*m | 1109 | XXXXXXXXXX | 20-mer 2′-OMethyl | DMD |
| 2331 | GUACCUCCAA | | A*mG*mG*mU*mA*mC*mC*mU*mC* | | XXXXXXXXX | | |
| | | | mC*mA*mA | | | | |
| WV- | UGCCAGAGCA | 358 | mU*mG*mC*mC*mA*mG*mA*mG*m | 1110 | XXXXXXXXXX | 20-mer 2′-OMethyl | DMD |
| 2332 | GGUACCUCCA | | C*mA*mG*mG*mU*mA*mC*mC*mU* | | XXXXXXXXX | | |
| | | | mC*mC*mA | | | | |
| WV- | CUGCCAGAGCA | 359 | mC*mU*mG*mC*mC*mA*mG*mA*m | 1111 | XXXXXXXXXX | 20-mer 2′-OMethyl | DMD |
| 2333 | GGUACCUCC | | G*mC*mA*mG*mG*mU*mA*mC*mC* | | XXXXXXXXX | | |
| | | | mU*mC*mC | | | | |
| WV- | UCUGCCAGAGC | 360 | mU*mC*mU*mG*mC*mC*mA*mG*m | 1112 | XXXXXXXXXX | 20-mer 2′-OMethyl | DMD |
| 2334 | AGGUACCUC | | A*mG*mC*mA*mG*mG*mU*mA*mC* | | XXXXXXXXX | | |
| | | | mC*mU*mC | | | | |
| WV- | AUCUGCCAGA | 361 | mA*mU*mC*mU*mG*mC*mC*mA*m | 1113 | XXXXXXXXXX | 20-mer 2′-OMethyl | DMD |
| 2335 | GCAGGUACCU | | G*mA*mG*mC*mA*mG*mG*mU*mA* | | XXXXXXXXX | | |
| | | | mC*mC*mU | | | | |
| WV- | UUGAAAUCUG | 362 | mU*mU*mG*mA*mA*mA*mU*mC*m | 1114 | XXXXXXXXXX | 20-mer 2′-OMethyl | DMD |
| 2336 | CCAGAGCAGG | | U*mG*mC*mC*mA*mG*mA*mG*mC* | | XXXXXXXXX | | |
| | | | mA*mG*mG | | | | |
| WV- | CCCGGUUGAA | 363 | mC*mC*mC*mG*mG*mU*mU*mG*m | 1115 | XXXXXXXXXX | 20-mer 2′-OMethyl | DMD |
| 2337 | AUCUGCCAGA | | A*mA*mA*mU*mC*mU*mG*mC*mC* | | XXXXXXXXX | | |
| | | | mA*mG*mA | | | | |
| WV- | GCCCGGUUGA | 364 | mG*mC*mC*mC*mG*mG*mU*mU*m | 1116 | XXXXXXXXXX | 20-mer 2′-OMethyl | DMD |
| 2338 | AAUCUGCCAG | | G*mA*mA*mA*mU*mC*mU*mG*mC* | | XXXXXXXXX | | |
| | | | mC*mA*mG | | | | |
| WV- | AGCCCGGUUG | 365 | mA*mG*mC*mC*mC*mG*mG*mU*m | 1117 | XXXXXXXXXX | 20-mer 2′-OMethyl | DMD |
| 2339 | AAAUCUGCCA | | U*mG*mA*mA*mA*mU*mC*mU*mG* | | XXXXXXXXX | | |
| | | | mC*mC*mA | | | | |
| WV- | CCAAGCCCGGU | 366 | mC*mC*mA*mA*mG*mC*mC*mC*m | 1118 | XXXXXXXXXX | 20-mer 2′-OMethyl | DMD |
| 2340 | UGAAAUCUG | | G*mG*mU*mU*mG*mA*mA*mA*mU | | XXXXXXXXX | | |
| | | | *mC*mU*mG | | | | |
| WV- | UCCAAGCCCGG | 367 | mU*mC*mC*mA*mA*mG*mC*mC*m | 1119 | XXXXXXXXXX | 20-mer 2′-OMethyl | DMD |
| 2341 | UUGAAAUCU | | C*mG*mG*mU*mU*mG*mA*mA*mA* | | XXXXXXXXX | | |
| | | | mU*mC*mU | | | | |
| WV- | GUCCAAGCCCG | 368 | mG*mU*mC*mC*mA*mA*mG*mC*m | 1120 | XXXXXXXXXX | 20-mer 2′-OMethyl | DMD |
| 2342 | GUUGAAAUC | | C*mC*mG*mG*mU*mU*mG*mA*mA* | | XXXXXXXXX | | |
| | | | mA*mU*mC | | | | |
| WV- | UGUCCAAGCCC | 369 | mU*mG*mU*mC*mC*mA*mA*mG*m | 1121 | XXXXXXXXXX | 20-mer 2′-OMethyl | DMD |
| 2343 | GGUUGAAAU | | C*mC*mC*mG*mG*mU*mU*mG*mA* | | XXXXXXXXX | | |
| | | | mA*mA*mU | | | | |
| WV- | CUGUCCAAGCC | 370 | mC*mU*mG*mU*mC*mC*mA*mA*m | 1122 | XXXXXXXXXX | 20-mer 2′-OMethyl | DMD |
| 2344 | CGGUUGAAA | | G*mC*mC*mC*mG*mG*mU*mU*mG* | | XXXXXXXXX | | |
| | | | mA*mA*mA | | | | |
| WV- | UCUGUCCAAGC | 371 | mU*mC*mU*mG*mU*mC*mC*mA*m | 1123 | XXXXXXXXXX | 20-mer 2′-OMethyl | DMD |
| 2345 | CCGGUUGAA | | A*mG*mC*mC*mC*mG*mG*mU*mU* | | XXXXXXXXX | | |
| | | | mG*mA*mA | | | | |
| WV- | UUCUGUCCAA | 372 | mU*mU*mC*mU*mG*mU*mC*mC*m | 1124 | XXXXXXXXXX | 20-mer 2′-OMethyl | DMD |
| 2346 | GCCCGGUUGA | | A*mA*mG*mC*mC*mC*mG*mG*mU* | | XXXXXXXXX | | |
| | | | mU*mG*mA | | | | |
| WV- | GUUCUGUCCA | 373 | mG*mU*mU*mC*mU*mG*mU*mC*m | 1125 | XXXXXXXXXX | 20-mer 2′-OMethyl | DMD |
| 2347 | AGCCCGGUUG | | C*mA*mA*mG*mC*mC*mC*mG*mG* | | XXXXXXXXX | | |
| | | | mU*mU*mG | | | | |
| WV- | AGUUCUGUCC | 374 | mA*mG*mU*mU*mC*mU*mG*mU*m | 1126 | XXXXXXXXXX | 20-mer 2′-OMethyl | DMD |
| 2348 | AAGCCCGGUU | | C*mC*mA*mA*mG*mC*mC*mC*mG* | | XXXXXXXXX | | |
| | | | mG*mU*mU | | | | |
| WV- | AAGUUCUGUC | 375 | mA*mA*mG*mU*mU*mC*mU*mG*m | 1127 | XXXXXXXXXX | 20-mer 2′-OMethyl | DMD |
| 2349 | CAAGCCCGGU | | U*mC*mC*mA*mA*mG*mC*mC*mC* | | XXXXXXXXX | | |
| | | | mG*mG*mU | | | | |
| WV- | UAAGUUCUGU | 376 | mU*mA*mA*mG*mU*mU*mC*mU*m | 1128 | XXXXXXXXXX | 20-mer 2′-OMethyl | DMD |
| 2350 | CCAAGCCCGG | | G*mU*mC*mC*mA*mA*mG*mC*mC* | | XXXXXXXXX | | |
| | | | mC*mG*mG | | | | |
| WV- | GUAAGUUCUG | 377 | mG*mU*mA*mA*mG*mU*mU*mC*m | 1129 | XXXXXXXXXX | 20-mer 2′-OMethyl | DMD |
| 2351 | UCCAAGCCCG | | U*mG*mU*mC*mC*mA*mA*mG*mC* | | XXXXXXXXX | | |
| | | | mC*mC*mG | | | | |
| WV- | GGUAAGUUCU | 378 | mG*mG*mU*mA*mA*mG*mU*mU*m | 1130 | XXXXXXXXXX | 20-mer 2′-OMethyl | DMD |
| 2352 | GUCCAAGCCC | | C*mU*mG*mU*mC*mC*mA*mA*mG* | | XXXXXXXXX | | |
| | | | mC*mC*mC | | | | |
| WV- | CAGUCGGUAA | 379 | mC*mA*mG*mU*mC*mG*mG*mU*m | 1131 | XXXXXXXXXX | 20-mer 2′-OMethyl | DMD |
| 2353 | GUUCUGUCCA | | A*mA*mG*mU*mU*mC*mU*mG*mU* | | XXXXXXXXX | | |
| | | | mC*mC*mA | | | | |
| WV- | CCAGUCGGUA | 380 | mC*mC*mA*mG*mU*mC*mG*mG*m | 1132 | XXXXXXXXXX | 20-mer 2′-OMethyl | DMD |
| 2354 | AGUUCUGUCC | | U*mA*mA*mG*mU*mU*mC*mU*mG* | | XXXXXXXXX | | |
| | | | mU*mC*mC | | | | |
| WV- | CCACCAUCACC | 381 | mC*mC*mA*mC*mC*mA*mU*mC*m | 1133 | XXXXXXXXXX | 20-mer 2′-OMethyl | DMD |
| 2355 | CUCUGUGAU | | A*mC*mC*mC*mU*mC*mU*mG*mU* | | XXXXXXXXX | | |
| | | | mG*mA*mU | | | | |
| WV- | CCCACCAUCAC | 382 | mC*mC*mC*mA*mC*mC*mA*mU*mC | 1134 | XXXXXXXXXX | 20-mer 2′-OMethyl | DMD |
| 2356 | CCUCUGUGA | | *mA*mC*mC*mC*mU*mC*mU*mG*m | | XXXXXXXXX | | |
| | | | U*mG*mA | | | | |
| WV- | CACCCACCAUC | 383 | mC*mA*mC*mC*mC*mA*mC*mC*mA | 1135 | XXXXXXXXXX | 20-mer 2′-OMethyl | DMD |
| 2357 | ACCCUCUGU | | *mU*mC*mA*mC*mC*mC*mU*mC*m | | XXXXXXXXX | | |
| | | | U*mG*mU | | | | |
| WV- | UCACCCACCAU | 384 | mU*mC*mA*mC*mC*mC*mA*mC*mC | 1136 | XXXXXXXXXX | 20-mer 2′-OMethyl | DMD |
| 2358 | CACCCUCUG | | *mA*mU*mC*mA*mC*mC*mC*mU*m | | XXXXXXXXX | | |
| | | | C*mU*mG | | | | |
| WV- | GUCACCCACCA | 385 | mG*mU*mC*mA*mC*mC*mC*mA*m | 1137 | XXXXXXXXXX | 20-mer 2′-OMethyl | DMD |
| 2359 | UCACCCUCU | | C*mC*mA*mU*mC*mA*mC*mC*mC* | | XXXXXXXXX | | |
| | | | mU*mC*mU | | | | |
| WV- | GGUCACCCACC | 386 | mG*mG*mU*mC*mA*mC*mC*mC*m | 1138 | XXXXXXXXXX | 20-mer 2′-OMethyl | DMD |
| 2360 | AUCACCCUC | | A*mC*mC*mA*mU*mC*mA*mC*mC* | | XXXXXXXXX | | |
| | | | mC*mU*mC | | | | |
| WV- | UCAAGCAGAG | 387 | mU*mC*mA*mA*mG*mC*mA*mG*m | 1139 | XXXXXXXXXX | 20-mer 2′-OMethyl | DMD |
| 2361 | AAAGCCAGUC | | A*mG*mA*mA*mA*mG*mC*mC*mA* | | XXXXXXXXX | | |
| | | | mG*mU*mC | | | | |
| WV- | UUGAUCAAGC | 388 | mU*mU*mG*mA*mU*mC*mA*mA*m | 1140 | XXXXXXXXXX | 20-mer 2′-OMethyl | DMD |
| 2362 | AGAGAAAGCC | | G*mC*mA*mG*mA*mG*mA*mA*mA* | | XXXXXXXXX | | |
| | | | mG*mC*mC | | | | |
| WV- | CAAAGAAGAU | 389 | mC*mA*mA*mA*mG*mA*mA*mG*m | 1141 | XXXXXXXXXX | based on WV-2223 | DMD |
| 2625 | GGCAUUUCUA | | A*mU*mG*mG*mC*mA*mU*mU*mU* | | XXXXXXXXXX | match mouse target | |
| | GUUUG | | mC*mU*mA*mG*mU*mU*mU*mG | | XXXX | sequence | |
| WV- | GCAAAGAAGA | 390 | mG*mC*mA*mA*mA*mG*mA*mA*m | 1142 | XXXXXXXXXX | based on WV-942 match | DMD |
| 2627 | UGGCAUUUCU | | G*mA*mU*mG*mG*mC*mA*mU*mU* | | XXXXXXXXX | mouse target sequence | |
| | | | mU*mC*mU | | | | |
| WV- | GCAAAGAAGA | 391 | fG*fC*fA*fA*fA*fG*mA*mA*mG*mA | 1143 | XXXXXXXXXX | based on WV-1714 | DMD |
| 2628 | UGGCAUUUCU | | *mU*mG*mG*mC*fA*fU*fU*fU*fC*fU | | XXXXXXXXX | match mouse target | |
| | | | | | | sequence | |
| WV- | UCAAGGAAGA | 392 | fU*fC*fA*fA*fG*mG*mA*mA*mG*m | 1144 | XXXXXXXXXX | Exon51: 5F -10OMe-5F | DMD |
| 2095 | UGGCAUUUCU | | A*mU*mG*mG*mC*mA*fU*fU*fU*fC | | XXXXXXXXX | all-PS | Exon51 |
| | | | *fU | | | | |
| WV- | UCAAGGAAGA | 393 | fU*fC*fA*fA*mG*mG*mA*mA*mG*m | 1145 | XXXXXXXXXX | Exon51: 4F-12OMe-4F | DMD |
| 2096 | UGGCAUUUCU | | A*mU*mG*mG*mC*mA*mU*fU*fU*f | | XXXXXXXXX | all-PS | Exon51 |
| | | | C*fU | | | | |
| WV- | UCAAGGAAGA | 394 | fU*fC*fA*mA*mG*mG*mA*mA*mG* | 1146 | XXXXXXXXXX | Exon51: 3F -14OMe-3F | DMD |
| 2097 | UGGCAUUUCU | | mA*mU*mG*mG*mC*mA*mU*mU*fU | | XXXXXXXXX | all-PS | Exon51 |
| | | | *fC*fU | | | | |
| WV- | UCAAGGAAGA | 395 | fU*fC*mA*mA*mG*mG*mA*mA*mG* | 1147 | XXXXXXXXXX | Exon51: 2F -16OMe-2F | DMD |
| 2098 | UGGCAUUUCU | | mA*mU*mG*mG*mC*mA*mU*mU*m | | XXXXXXXXX | all-PS | Exon51 |
| | | | U*fC*fU | | | | |
| WV- | UCAAGGAAGA | 396 | fU*mC*mA*mA*mG*mG*mA*mA*mG | 1148 | XXXXXXXXXX | Exon51: 1F-18OMe-1F | DMD |
| 2099 | UGGCAUUUCU | | *mA*mU*mG*mG*mC*mA*mU*mU* | | XXXXXXXXX | all-PS | Exon51 |
| | | | mU*mC*fU | | | | |
| WV- | UCAAGGAAGA | 397 | fU*fC*fA*fA*fG*fGmA*mA*mG*mA* | 1149 | XXXXXOXXXX | Exon51: 6F-8OMe-6F | DMD |
| 2100 | UGGCAUUUCU | | mU*mG*mG*mCfA*fU*fU*fU*fC*fU | | XXXOXXXXX | 5PS-1PO-7PS-1PO-5PS | Exon51 |
| WV- | UCAAGGAAGA | 398 | fU*fC*fA*fA*fGfGmA*mA*mG*mA*m | 1150 | XXXXOOXXXX | Exon51: 6F-8OMe-6F | DMD |
| 2101 | UGGCAUUUCU | | U*mG*mG*mCfAfU*fU*fU*fC*fU | | XXXOOXXXX | 4PS-2PO-7PS-2PO-4PS | Exon51 |
| WV- | UCAAGGAAGA | 399 | fU*fC*fA*fAfGfGmA*mA*mG*mA*m | 1151 | XXXOOOXXXX | Exon51: 6F-8OMe-6F | DMD |
| 2102 | UGGCAUUUCU | | U*mG*mG*mCfAfUfU*fU*fC*fU | | XXXOOOXXX | 3PS-3PO-7PS-3PO-3PS | Exon51 |
| WV- | UCAAGGAAGA | 400 | fU*fC*fAfAfGfGmA*mA*mG*mA*mU | 1152 | XXOOOOXXXX | Exon51: 6F-8OMe-6F | DMD |
| 2103 | UGGCAUUUCU | | *mG*mG*mCfAfUfUfU*fC*fU | | XXXOOOOXX | 2PS-4PO-7PS-4PO-2PS | Exon51 |
| WV- | UCAAGGAAGA | 401 | fU*fCfAfAfGfGmA*mA*mG*mA*mU* | 1153 | XOOOOOXXXX | Exon51: 6F-8OMe-6F | DMD |
| 2104 | UGGCAUUUCU | | mG*mG*mCfAfUfUfUfC*fU | | XXXOOOOOX | 1PS-5PO-7PS-5PO-1PS | Exon51 |
| WV- | UCAAGGAAGA | 402 | fUfCfAfAfGfGmA*mA*mG*mA*mU*m | 1154 | OOOOOOXXXX | Exon51: 6F-8OMe-6F | DMD |
| 2105 | UGGCAUUUCU | | G*mG*mCfAfUfUfUfCfU | | XXXOOOOOO | 6PO-7PS-6PO | Exon51 |
| WV- | UCAAGGAAGA | 403 | fU*fC*fA*fA*fG*fG*fA*fA*fG*fA*mU | 1155 | XXXXXXXXXX | Exon51: 10F-10OMe | DMD |
| 2106 | UGGCAUUUCU | | *mG*mG*mC*mA*mU*mU*mU*mC*mU | | XXXXXXXXX | all-PS | Exon51 |
| WV- | UCAAGGAAGA | 404 | mU*mC*mA*mA*mG*mG*mA*mA*m | 1156 | XXXXXXXXXX | Exon51: 10OMe-10F | DMD |
| 2107 | UGGCAUUUCU | | G*mA*fU*fG*fG*fC*fA*fU*fU*fU*fC* | | XXXXXXXXX | all-PS | Exon51 |
| | | | fU | | | | |
| WV- | UCAAGGAAGA | 405 | fU*fC*fA*fA*fG*fG*mA*mA*mG*mA | 1157 | XXXXXXXXXX | Exon51: 6F-14OMe all- | DMD |
| 2108 | UGGCAUUUCU | | *mU*mG*mG*mC*mA*mU*mU*mU* | | XXXXXXXXX | PS | Exon51 |
| | | | mC*mU | | | | |
| WV- | UCAAGGAAGA | 406 | mU*mC*mA*mA*mG*mG*mA*mA*m | 1158 | XXXXXXXXXX | Exon51: 14OMe-6F all- | DMD |
| 2109 | UGGCAUUUCU | | G*mA*mU*mG*mG*mC*fA*fU*fU*fU | | XXXXXXXXX | PS | Exon51 |
| | | | *fC*fU | | | | |
| WV-884 | UCAAGGAAGA | 407 | mU*RmC*RmA*RmA*RmG*RmG*Rm | 1159 | RRRRRRRRRRR | All-R; 2′-OMe oligo | Dystrophin |
| | UGGCAUUUCU | | A*RmA*RmG*RmA*RmU*RmG*RmG | | RRRRRRRR | | |
| | | | *RmC*RmA*RmU*RmU*RmU*RmC*R | | | | |
| | | | mU | | | | |
| WV-885 | UCAAGGAAGA | 408 | mU*SmC*RmA*SmA*RmG*SmG*RmA | 1160 | SRSRSRSRSRSR | (SR)9S; 2′-OMe oligo | Dystrophin |
| | UGGCAUUUCU | | *SmA*RmG*SmA*RmU*SmG*RmG*S | | SRSRSRS | | |
| | | | mC*RmA*SmU*RmU*SmU*RmC*SmU | | | | |
| WV-886 | UCAAGGAAGA | 409 | mU*RmC*RmA*RmA*SmG*SmG*SmA | 1161 | RRRSSSSSSSSSS | R3S13R3; 2′-OMe oligo | Dystrophin |
| | UGGCAUUUCU | | *SmA*SmG*SmA*SmU*SmG*SmG*Sm | | SSSRRR | | |
| | | | C*SmA*SmU*SmU*RmU*RmC*RmU | | | | |
| WV-887 | UCAAGGAAGA | 410 | mU*SmC*SmA*SmA*RmG*RmG*RmA | 1162 | SSSRRRRRRRRR | S3R13S3; 2′-OMe oligo | Dystrophin |
| | UGGCAUUUCU | | *RmA*RmG*RmA*RmU*RmG*RmG*R | | RRRRSSS | | |
| | | | mC*RmA*RmU*RmU*SmU*SmC*SmU | | | | |
| WV-888 | UCAAGGAAGA | 411 | mU*RmC*RmA*RmA*RmG*RmG*Sm | 1163 | RRRRRSSRSSRS | R5(SSR)3R5; 2′-OMe | Dystrophin |
| | UGGCAUUUCU | | A*SmA*RmG*SmA*SmU*RmG*SmG* | | SRRRRRR | oligo | |
| | | | SmC*RmA*RmU*RmU*RmU*RmC*R | | | | |
| | | | mU | | | | |
| WV-889 | UCAAGGAAGA | 412 | mU*SmC*SmA*SmA*SmG*SmG*RmA | 1164 | SSSSSRRSRRSR | S5(RRS)3S5; 2′-OMe | Dystrophin |
| | UGGCAUUUCU | | *RmA*SmG*RmA*RmU*SmG*RmG*R | | RSSSSSS | oligo | |
| | | | mC*SmA*SmU*SmU*SmU*SmC*SmU | | | | |
| WV-890 | UCAAGGAAGA | 413 | mU*RmC*RmA*RmA*SmG*SmG*Rm | 1165 | RRRSSRRSRRRS | R3S2R2SR3SR2S2R3; | Dystrophin |
| | UGGCAUUUCU | | A*RmA*SmG*RmA*RmU*RmG*SmG* | | RRSSRRR | 2′-OMe oligo | |
| | | | RmC*RmA*SmU*SmU*RmU*RmC*RmU | | | | |
| WV-891 | UCAAGGAAGA | 414 | mU*SmC*SmA*SmA*RmG*RmG*SmA | 1166 | SSSRRSSRSSSRS | S3R2S2RS3RS2R2S3; | Dystrophin |
| | UGGCAUUUCU | | *SmA*RmG*SmA*SmU*SmG*RmG*S | | SRRSSS | 2′-OMe oligo | |
| | | | mC*SmA*RmU*RmU*SmU*SmC*SmU | | | | |
| WV-892 | UCAAGGAAGA | 415 | mU*SmC*RmA*RmA*RmG*RmG*Rm | 1167 | SRRRRRRRRRR | SR17S; 2′-OMe | Dystrophin |
| | UGGCAUUUCU | | A*RmA*RmG*RmA*RmU*RmG*RmG | | RRRRRRRS | chimeric oligo | |
| | | | *RmC*RmA*RmU*RmU*RmU*RmC*S | | | | |
| | | | mU | | | | |
| WV-893 | UCAAGGAAGA | 416 | mU*RmC*SmA*SmA*SmG*SmG*SmA | 1168 | RSSSSSSSSSSSS | RS17R; 2′-OMe | Dystrophin |
| | UGGCAUUUCU | | *SmA*SmG*SmA*SmU*SmG*SmG*Sm | | SSSSSR | chimeric oligo | |
| | | | C*SmA*SmU*SmU*SmU*SmC*RmU | | | | |
| WV-894 | UCAAGGAAGA | 417 | mU*SmC*RmA*SmA*SmG*RmG*RmA | 1169 | SRSSRRSSRSSR | GC(R) and AU(S) 2′- | Dystrophin |
| | UGGCAUUUCU | | *SmA*SmG*RmA*SmU*SmG*RmG*R | | RRSSSSR | OMe oligo | |
| | | | mC*RmA*SmU*SmU*SmU*SmC*RmU | | | | |
| WV-895 | UCAAGGAAGA | 418 | mU*RmC*SmA*RmA*RmG*SmG*SmA | 1170 | RSRRSSRRSRRS | GC(S) and AU(R) 2′- | Dystrophin |
| | UGGCAUUUCU | | *RmA*RmG*SmA*RmU*RmG*SmG*S | | SSRRRRS | OMe oligo | |
| | | | mC*SmA*RmU*RmU*RmU*RmC*SmU | | | | |
| WV-896 | UCAAGGAAGA | 419 | mU*SmC*SmA*RmA*RmG*RmG*Rm | 1171 | SSRRRRRRRRSR | GA(R) and CU(S) 2′- | Dystrophin |
| | UGGCAUUUCU | | A*RmA*RmG*RmA*RmU*SmG*RmG* | | RSRSSSS | OMe oligo | |
| | | | RmC*SmA*RmU*SmU*SmU*SmC*SmU | | | | |
| WV-897 | UCAAGGAAGA | 420 | mU*RmC*RmA*SmA*SmG*SmG*SmA | 1172 | RRSSSSSSSSRSS | GA(S) and CU(R) 2′- | Dystrophin |
| | UGGCAUUUCU | | *SmA*SmG*SmA*SmU*RmG*SmG*S | | RSRRRR | OMe oligo | |
| | | | mC*RmA*SmU*RmU*RmU*RmC*RmU | | | | |
| WV- | GGCCAAACCUC | 421 | fG*fG*fC*fC*fA*fA*fA*fC*fC*fU*fC*f | 1173 | XXXXXXXXXX | All 2′-F modified | Exon 23 |
| 1678 | GGCUUACCU | | G*fG*fC*fU*fU*fA*fC*fC*fU | | XXXXXXXXX | | |
| WV- | GGCCAAACCUC | 422 | mG*mG*fC*fC*mA*mA*mA*fC*fC*fU | 1174 | XXXXXXXXXX | 2′-F pyrimidines; 2′- | Exon 23 |
| 1679 | GGCUUACCU | | *fC*mG*mG*fC*fU*fU*mA*fC*fC*fU | | XXXXXXXXX | OMe purines | |
| WV- | GGCCAAACCUC | 423 | fG*fG*mC*mC*fA*fA*fA*mC*mC*mU | 1175 | XXXXXXXXXX | 2′-F purines; 2′-OMe | Exon 23 |
| 1680 | GGCUUACCU | | *mC*fG*fG*mC*mU*mU*fA*mC*mC* | | XXXXXXXXX | pyrimidines | |
| | | | mU | | | | |
| WV- | GGCCAAACCUC | 424 | mG*fG*mC*fC*mA*fA*mA*fC*mC*fU | 1176 | XXXXXXXXXX | Alternate 2′-OMe/2′F | Exon 23 |
| 1681 | GGCUUACCU | | *mC*fG*mG*fC*mU*fU*mA*fC*mC*fU | | XXXXXXXXX | | |
| WV- | GGCCAAACCUC | 425 | mG*mG*mC*mC*mA*mA*fA*fC*fC*f | 1177 | XXXXXXXXXX | 2′-OMe/2′-F/2′-OMe | Exon 23 |
| 1682 | GGCUUACCU | | U*fC*fG*fG*fC*mU*mU*mA*mC*mC | | XXXXXXXXX | gapmer | |
| | | | *mU | | | | |
| WV- | GGCCAAACCUC | 426 | fG*fG*fC*fC*fA*fA*mA*mC*mC*mU* | 1178 | XXXXXXXXXX | 2′-F/2′-OMe/2′-F gapmer | Exon 23 |
| 1683 | GGCUUACCU | | mC*mG*mG*mC*fU*fU*fA*fC*fC*fU | | XXXXXXXXX | | |
| WV- | GGCCAAACCUC | 427 | fG*fG*fC*fC*mA*mA*mA*fC*fC*mU* | 1179 | XXXXXXXXXX | 2′-F (C; G); 2′-OMe (U; | Exon 23 |
| 1684 | GGCUUACCU | | fC*fG*fG*fC*mU*mU*mA*fC*fC*mU | | XXXXXXXXX | A) | |
| WV- | GGCCAAACCUC | 428 | mG*mG*mC*mC*fA*fA*fA*mC*mC*f | 1180 | XXXXXXXXXX | 2′-F (U; A); 2′-OMe (C; | Exon 23 |
| 1685 | GGCUUACCU | | U*mC*mG*mG*mC*fU*fU*fA*mC*mC | | XXXXXXXXX | G) | |
| | | | *fU | | | | |
| WV- | UCAAGGAAGA | 429 | fU*fC*fA*fA*fG*fG*fA*fA*fG*fA*fU* | 1181 | XXXXXXXXXX | | Exon 51 |
| 1709 | UGGCAUUUCU | | fG*fG*fC*fA*fU*fU*fU*fC*fU | | XXXXXXXXX | | |
| WV- | UCAAGGAAGA | 430 | fU*fC*mA*mA*mG*mG*mA*mA*mG* | 1182 | XXXXXXXXXX | | Exon 51 |
| 1710 | UGGCAUUUCU | | mA*fU*mG*mG*fC*mA*fU*fU*fU*fC | | XXXXXXXXX | | |
| | | | *fu | | | | |
| WV- | UCAAGGAAGA | 431 | mU*mC*fA*fA*fG*fG*fA*fA*fG*fA*m | 1183 | XXXXXXXXXX | | Exon 51 |
| 1711 | UGGCAUUUCU | | U*fG*fG*mC*fA*mU*mU*mU*mC*mU | | XXXXXXXXX | | |
| WV- | UCAAGGAAGA | 432 | mU*fC*mA*fA*mG*fG*mA*fA*mG*f | 1184 | XXXXXXXXXX | | Exon 51 |
| 1712 | UGGCAUUUCU | | A*mU*fG*mG*fC*mA*fU*mU*fU*mC | | XXXXXXXXX | | |
| | | | *fu | | | | |
| WV- | UCAAGGAAGA | 433 | mU*mC*mA*mA*mG*mG*fA*fA*fG*f | 1185 | XXXXXXXXXX | | Exon 51 |
| 1713 | UGGCAUUUCU | | A*fU*fG*fG*fC*mA*mU*mU*mU*mC | | XXXXXXXXX | | |
| | | | *mU | | | | |
| WV- | UCAAGGAAGA | 434 | fU*fC*fA*fA*fG*fG*mA*mA*mG*mA | 1186 | XXXXXXXXXX | | Exon 51 |
| 1714 | UGGCAUUUCU | | *mU*mG*mG*mC*fA*fU*fUqU*fC*fU | | XXXXXXXXX | | |
| WV- | UCAAGGAAGA | 435 | mU*fC*mA*mA*fG*fG*mA*mA*fG*m | 1187 | XXXXXXXXXX | | Exon 51 |
| 1715 | UGGCAUUUCU | | A*mU*fG*fG*fC*mA*mU*mU*mU*fC | | XXXXXXXXX | | |
| | | | *mU | | | | |
| WV- | UCAAGGAAGA | 436 | fU*mC*fA*fA*mG*mG*fA*fA*mG*fA | 1188 | XXXXXXXXXX | | Exon 51 |
| 1716 | UGGCAUUUCU | | *fU*mG*mG*mC*fA*fU*fU*fU*mC*fU | | XXXXXXXXX | | |
| WV- | GGCCAAACCTC | 437 | G*G*C*C*A*A*A*C*C*T*C*G*G*C* | 1189 | XXXXXXXXXX | Stereorandom DNA | Exon23 |
| 1093 | GGCTTACCT | | T*T*A*C*C*T | | XXXXXXXXX | version of Exon23 full | |
| | | | | | | PS: Analog of WV943 | |
| WV- | GGCCAAACCUC | 438 | mGmGmCmCmAmAmAmCmCmUmCm | 1190 | OOOOOOOOOO | Full PO version of | Exon23 |
| 1094 | GGCUUACCU | | GmGmCmUmUmAmCmCmU | | OOOOOOOOO | WV943 | |
| WV- | GGCCAAACCTC | 439 | G*RG*RC*RC*RA*RA*RA*RC*RC*R | 1191 | RRRRRRRRRRR | Full Rp DNA version of | Exon23 |
| 1095 | GGCTTACCT | | T*RC*RG*RG*RC*RT*RT*RA*RC*RC | | RRRRRRRR | Exon23: Analog of | |
| | | | *RT | | | WV943 | |
| WV- | GGCCAAACCTC | 440 | G*SG*SC*SC*SA*SA*SA*SC*SC*ST* | 1192 | SSSSSSSSSSSSSS | Full Sp DNA version of | Exon23 |
| 1096 | GGCTTACCT | | SC*SG*SG*SC*ST*ST*SA*SC*SC*ST | | SSSSS | Exon23: Analog of | |
| | | | | | | WV943 | |
| WV- | GGCCAAACCUC | 441 | G*SG*SC*SC*SA*SmAmAmCmCmUm | 1193 | SSSSSOOOOOO | Stereopure DNA/2′OMe | Exon23 |
| 1097 | GGCTTACCT | | CmGmGmC T*S T*SA*SC*SC*ST | | OOOSSSSS | chimeric version of | |
| | | | | | | Exon23: Analog of 943 | |
| WV- | GGCCAAACCTC | 442 | mGmGmCmCA*SA*SA*SmCC*ST*SC | 1194 | OOOOSSSOSSSS | Stereopure DNA/2′OMe | Exon23 |
| 1098 | GGCTTACCU | | *SG*SmGC*ST*ST*SmAmCmCmU | | OSSSOOO | chimeric version of | |
| | | | | | | Exon23: Analog of 943 | |
| WV- | GGCCAAACCUC | 443 | G*SmGC*SmCA*SmAA*SmCC*SmUC | 1195 | SOSOSOSOSOSO | Stereopure DNA/2′OMe | Exon23 |
| 1099 | GGCTUACCU | | *SmGG*SmCT*SmUA*SmCC*SmU | | SOSOSOS | chimeric version of | |
| | | | | | | Exon23: Analog of 943 | |
| WV- | GGCCAAACCTC | 444 | mGG*SmCC*SmAA*SmAC*SmCT*Sm | 1196 | OSOSOSOSOSOS | Stereopure DNA/2′OMe | Exon23 |
| 1100 | GGCUTACCU | | CG*SmGC*SmUT*SmAC*SmCmU | | OSOSOSO | chimeric version of | |
| | | | | | | Exon23: Analog of 943 | |
| WV- | GGCCAAACCTC | 445 | G*SG*SmCmCA*SA*SmAmCC*ST*SC | 1197 | SSOOSSOOSSSO | Stereopure DNA/2′OMe | Exon23 |
| 1101 | GGCTUACCU | | *SmGmGC*ST*SmUmAC*SC*SmU | | OSSOOSS | chimeric version of | |
| | | | | | | Exon23: Analog of 943 | |
| WV- | GGCCAAACCUC | 446 | G*SG*SC*SmCmAmAA*SC*SmCmUm | 1198 | SSSOOOSSOOOS | Stereopure DNA/2′OMe | Exon23 |
| 1102 | GGCUUACCU | | CG*SG*SmCmUmUA*SC*SC*SmU | | SOOOSSS | chimeric version of | |
| | | | | | | Exon23: Analog of 943 | |
| WV- | GGCCAAACCTC | 447 | G*SG*SC*SC*SmAmAmAmCC*ST*SC | 1199 | SSSSOOOOSSSO | Stereopure DNA/2′OMe | Exon23 |
| 1103 | GGCUTACCU | | *SmGmGmCmUT*SA*SC*SC*SmU | | OOOSSSS | chimeric version of | |
| | | | | | | Exon23: Analog of 943 | |
| WV- | GGCCAAACCTC | 448 | G*SG*SC*SmCA*SA*SA*SmCC*ST*S | 1200 | SSSOSSSOSSSOS | Stereopure DNA/2′OMe | Exon23 |
| 1104 | GGCTUACCU | | C*SmGG*SC*ST*SmUA*SC*SC*SmU | | SSOSSS | chimeric version of | |
| | | | | | | Exon23: Analog of 943 | |
| WV- | GGCCAAACCUC | 449 | mGmGmCmCA*SA*SA*SC*SC*SmUm | 1201 | OOOOSSSSSOO | Stereopure DNA/2′OMe | Exon23 |
| 1105 | GGCTTACCU | | CmGmGmCT*ST*SA*SC*SC*SmU | | OOOSSSS | chimeric version of | |
| | | | | | | Exon23: Analog of 943 | |
| WV- | GGCCAAACCUC | 450 | G*G*C*C*A*mAmAmCmCmUmCmGm | 1202 | XXXXXOOOOO | Stereorandom | Exon23 |
| 1121 | GGCTTACCT | | GmCT*T*A*C*C*T | | OOOOXXXXX | DNA/2′OMe chimeric | |
| | | | | | | version of Exon23: | |
| | | | | | | Analog of WV1097 | |
| WV- | GGCCAAACCTC | 451 | mGmGmCmCA*A*A*mCC*T*C*G*mG | 1203 | OOOOXXXOXX | Stereorandom | Exon23 |
| 1122 | GGCTTACCU | | C*T*T*mAmCmCmU | | XXOXXXOOO | DNA/2′OMe chimeric | |
| | | | | | | version of Exon23: | |
| | | | | | | Analog of WV1098 | |
| WV- | GGCCAAACCUC | 452 | G*mGC*mCA*mAA*mCC*mUC*mGG | 1204 | XOXOXOXOXO | Stereorandom | Exon23 |
| 1123 | GGCTUACCU | | *mCT*mUA*mCC*mU | | XOXOXOXOX | DNA/2′OMe chimeric | |
| | | | | | | version of Exon23: | |
| | | | | | | Analog of WV1099 | |
| WV- | GGCCAAACCTC | 453 | mGG*mCC*mAA*mAC*mCT*mCG*m | 1205 | OXOXOXOXOX | Stereorandom | Exon23 |
| 1124 | GGCUTACCU | | GC*mUT*mAC*mCmU | | OXOXOXOXO | DNA/2′OMe chimeric | |
| | | | | | | version of Exon23: | |
| | | | | | | Analog of WV11OO | |
| WV- | GGCCAAACCTC | 454 | G*G*mCmCA*A*mAmCmCT*C*mGm | 1206 | XXOOXXOOOX | Stereorandom | Exon23 |
| 1125 | GGCTUACCU | | GC*T*mUmAC*C*mU | | XOOXXOOXX | DNA/2′OMe chimeric | |
| | | | | | | version of Exon23: | |
| | | | | | | Analog of WV1101 | |
| WV- | GGCCAAACCUC | 455 | G*G*C*mCmAmAA*C*mCmUmCG*G | 1207 | XXXOOOXXOO | Stereorandom | Exon23 |
| 1126 | GGCUUACCU | | *mCmUmUA*C*C*mU | | OXXOOOXXX | DNA/2′OMe chimeric | |
| | | | | | | version of Exon23: | |
| | | | | | | Analog of WV1102 | |
| WV- | GGCCAAACCTC | 456 | G*G*C*C*mAmAmAmCC*T*C*mGmG | 1208 | XXXXOOOOXX | Stereorandom | Exon23 |
| 1127 | GGCUTACCU | | mCmUT*A*C*C*mU | | XOOOOXXXX | DNA/2′OMe chimeric | |
| | | | | | | version of Exon23: | |
| | | | | | | Analog of WV1103 | |
| WV- | GGCCAAACCTC | 457 | G*G*C*mCA*A*A*mCC*T*C*mGG*C | 1209 | XXXOXXXOXX | Stereorandom | Exon23 |
| 1128 | GGCTUACCU | | *T*mUA*C*C*mU | | XOXXXOXXX | DNA/2′OMe chimeric | |
| | | | | | | version of Exon23: | |
| | | | | | | Analog of WV1104 | |
| WV- | GGCCAAACCUC | 458 | mGmGmCmCA*A*A*C*C*mUmCmGm | 1210 | OOOOXXXXXO | Stereorandom | Exon23 |
| 1129 | GGCTTACCU | | GmCT*T*A*C*C*mU | | OOOOXXXXX | DNA/2′OMe chimeric | |
| | | | | | | version of Exon23: | |
| | | | | | | Analog of WV1105 | |
| WV- | GGCCAAACCUC | 459 | G*G*mCmCmAmAmAmCmCmUC*mG | 1211 | XXOOOOOOOO | Stereorandom | Exon23 |
| 1130 | GGCUTACCU | | mGC*mUT*A*C*C*mU | | XOOXOXXXX | DNA/2′OMe chimeric | |
| | | | | | | version of Exon23: | |
| | | | | | | Analog of WV1106 | |
| WV- | GGCCAAACCUC | 460 | mG*mG*mC*mC*mA*mAmAmCmCm | 1212 | XXXXXOOOOO | Stereorandom 2′OMe | Exon23 |
| 1141 | GGCUUACCU | | UmCmGmGmCmU*mU*mA*mC*mC* | | OOOOXXXXX | PO/PS chimeric version | |
| | | | mU | | | of exon23: Analog of | |
| | | | | | | WV1097 | |
| WV- | GGCCAAACCUC | 461 | mGmGmCmCmA*mA*mA*mCmC*mU | 1213 | OOOOXXXOXX | Stereorandom 2′OMe | Exon23 |
| 1142 | GGCUUACCU | | *mC*mG*mGmC*mU*mU*mAmCmCmU | | XXOXXXOOO | PO/PS chimeric version | |
| | | | | | | of exon23: Analog of | |
| | | | | | | WV1098 | |
| WV- | GGCCAAACCUC | 462 | mG*mGmC*mCmA*mAmA*mCmC*m | 1214 | XOXOXOXOXO | Stereorandom 2′OMe | Exon23 |
| 1143 | GGCUUACCU | | UmC*mGmG*mCmU*mUmA*mCmC* | | XOXOXOXOX | PO/PS chimeric version | |
| | | | mU | | | of exon23: Analog of | |
| | | | | | | WV1099 | |
| WV- | GGCCAAACCUC | 463 | mGmG*mCmC*mAmA*mAmC*mCmU | 1215 | OXOXOXOXOX | Stereorandom 2′OMe | Exon23 |
| 1144 | GGCUUACCU | | *mCmG*mGmC*mUmU*mAmC*mCmU | | OXOXOXOXO | PO/PS chimeric version | |
| | | | | | | of exon23: Analog of | |
| | | | | | | WV1100 | |
| WV- | GGCCAAACCUC | 464 | mG*mG*mCmCmA*mA*mAmCmCmU | 1216 | XXOOXXOOOX | Stereorandom 2′OMe | Exon23 |
| 1145 | GGCUUACCU | | *mC*mGmGmC*mU*mUmAmC*mC*mU | | XOOXXOOXX | PO/PS chimeric version | |
| | | | | | | of exon23: Analog of | |
| | | | | | | WV1101 | |
| WV- | GGCCAAACCUC | 465 | mG*mG*mC*mCmAmAmA*mC*mCm | 1217 | XXXOOOXXOO | Stereorandom 2′OMe | Exon23 |
| 1146 | GGCUUACCU | | UmCmG*mG*mCmUmUmA*mC*mC* | | OXXOOOXXX | PO/PS chimeric version | |
| | | | mU | | | of exon23: Analog of | |
| | | | | | | WV1102 | |
| WV- | GGCCAAACCUC | 466 | mG*mG*mC*mC*mAmAmAmCmC*m | 1218 | XXXXOOOOXX | Stereorandom 2′OMe | Exon23 |
| 1147 | GGCUUACCU | | U*mC*mGmGmCmUmU*mA*mC*mC* | | XOOOOXXXX | PO/PS chimeric version | |
| | | | mU | | | of exon23: Analog of | |
| | | | | | | WV1103 | |
| WV- | GGCCAAACCUC | 467 | mG*mG*mC*mCmA*mA*mA*mCmC* | 1219 | XXXOXXXOXX | Stereorandom 2′OMe | Exon23 |
| 1148 | GGCUUACCU | | mU*mC*mGmG*mC*mU*mUmA*mC* | | XOXXXOXXX | PO/PS chimeric version | |
| | | | mC*mU | | | of exon23: Analog of | |
| | | | | | | WV1104 | |
| WV- | GGCCAAACCUC | 468 | mGmGmCmCmA*mA*mA*mC*mC*m | 1220 | OOOOXXXXXO | Stereorandom 2′OMe | Exon23 |
| 1149 | GGCUUACCU | | UmCmGmGmCmU*mU*mA*mC*mC* | | OOOOXXXXX | PO/PS chimeric version | |
| | | | mU | | | of exon23: Analog of | |
| | | | | | | WV1105 | |
| WV- | GGCCAAACCUC | 469 | mG*mG*mCmCmAmAmAmCmCmUm | 1221 | XXOOOOOOOO | Stereorandom 2′OMe | Exon23 |
| 1150 | GGCUUACCU | | C*mGmGmC*mUmU*mA*mC*mC*mU | | XOOXOXXXX | PO/PS chimeric version | |
| | | | | | | of exon23: Analog of | |
| | | | | | | WV1106 | |
| WV- | GGCCAAACCUC | 470 | L001*mG*mG*mC*mC*mA*mA*mA* | 1222 | XXXXXXXXXX | All-OMe full-PS | Exon23 |
| 2733 | GGCUUACCU | | mC*mC*mU*mC*mG*mG*mC*mU*m | | XXXXXXXXXX | | |
| | | | U*mA*mC*mC*mU | | | | |
| WV- | GGCCAAACCUC | 471 | L001*mG*mG*mC*mC*mA*mA*mA* | 1223 | XXXXXXXXXX | All-OMe full-PS | Exon23 |
| 2734 | GGCUUACCUG | | mC*mC*mU*mC*mG*mG*mC*mU*m | | XXXXXXXXXX | | |
| | AAAU | | U*mA*mC*mC*mU*mG*mA*mA*mA* | | XXXXX | | |
| | | | mU | | | | |
| WV- | GGCCAAACCUC | 472 | G*SG*SmCmCmAmAmAmCmCmUC*S | 1224 | SSOOOOOOOOS | Stereopure DNA/2′OMe | Exon51 |
| 1106 | GGCUTACCU | | mGmGC*SmUT*SA*SC*SC*SmU | | OOSOSSSS | chimeric version of | |
| | | | | | | Exon23: Analog of 943 | |
| WV- | TCAAGGAAGAT | 473 | T*C*A*A*G*G*A*A*G*A*T*G*G*C* | 1225 | XXXXXXXXXX | Stereorandom DNA | Exon51 |
| 1107 | GGCATTTCT | | A*T*T*T*C*T | | XXXXXXXXX | version of Exon51 full | |
| | | | | | | PS: Analog of WV942 | |
| WV- | UCAAGGAAGA | 474 | mUmCmAmAmGmGmAmAmGmAmU | 1226 | OOOOOOOOOO | Full PO version of | Exon51 |
| 1108 | UGGCAUUUCU | | mGmGmCmAmUmUmUmCmU | | OOOOOOOOO | WV942 | |
| WV- | TCAAGGAAGAT | 475 | T*RC*RA*RA*RG*RG*RA*RA*RG*R | 1227 | RRRRRRRRRRR | Full Rp DNA version of | Exon51 |
| 1109 | GGCATTTCT | | A*RT*RG*RG*RC*RA*RT*RT*RT*RC | | RRRRRRRR | Exon51: Analog of | |
| | | | *RT | | | WV942 | |
| WV- | TCAAGGAAGAT | 476 | T*SC*SA*SA*SG*SG*SA*SA*SG*SA* | 1228 | SSSSSSSSSSSSSS | Full Rp DNA version of | Exon51 |
| 1110 | GGCATTTCT | | ST*SG*SG*SC*SA*ST*ST*ST*SC*ST | | SSSSS | Exon51: Analog of | |
| | | | | | | WV942 | |
| WV- | TCAAGGAAGA | 477 | T*SC*SA*SA*SG*SmGmAmAmGmAm | 1229 | SSSSSOOOOOO | Stereopure DNA/2′OMe | Exon51 |
| 1111 | UGGCATTTCT | | UmGmGmCA*ST*ST*ST*SC*ST | | OOOSSSSS | chimeric version of | |
| | | | | | | Exon51: Analog of 942 | |
| WV- | UCAAGGAAGA | 478 | mUmCmAmAG*SG*SA*SmAG*SA*ST | 1230 | OOOOSSSOSSSS | Stereopure DNA/2′OMe | Exon51 |
| 1112 | TGGCATUUCU | | *SG*SmGC*SA*ST*SmUmUmCmU | | OSSSOOO | chimeric version of | |
| | | | | | | Exon51: Analog of 942 | |
| WV- | TCAAGGAAGAT | 479 | T*SmCA*SmAG*SmGA*SmAG*SmAT | 1231 | SOSOSOSOSOSO | Stereopure DNA/2′OMe | Exon51 |
| 1113 | GGCAUTUCU | | *SmGG*SmCA*SmUT*SmUC*SmU | | SOSOSOS | chimeric version of | |
| | | | | | | Exon51: Analog of 942 | |
| WV- | UCAAGGAAGA | 480 | mUC*SmAA*SmGG*SmAA*SmGA*Sm | 1232 | OSOSOSOSOSOS | Stereopure DNA/2′OMe | Exon51 |
| 1114 | UGGCATUTCU | | UG*SmGC*SmAT*SmUT*SmCmU | | OSOSOSO | chimeric version of | |
| | | | | | | Exon51: Analog of 942 | |
| WV- | TCAAGGAAGAT | 481 | T*SC*SmAmAG*SG*SmAmAG*SA*ST | 1233 | S SOOSSOOSSSO | Stereopure DNA/2′OMe | Exon51 |
| 1115 | GGCAUUTCU | | *SmGmGC*SA*SmUmUT*SC*SmU | | OSSOOSS | chimeric version of | |
| | | | | | | Exon51: Analog of 942 | |
| WV- | TCAAGGAAGA | 482 | T*SC*SA*SmAmGmGA*SA*SmGmAm | 1234 | SSSOOOSSOOOS | Stereopure DNA/2′OMe | Exon51 |
| 1116 | UGGCAUTTCU | | UG*SG*SmCmAmUT*ST*SC*SmU | | SOOOSSS | chimeric version of | |
| | | | | | | Exon51: Analog of 942 | |
| WV- | TCAAGGAAGAT | 483 | T*SC*SA*SA*SmGmGmAmAG*SA*ST | 1235 | SSSSOOOOSSSO | Stereopure DNA/2′OMe | Exon51 |
| 1117 | GGCATTTCU | | *SmGmGmCmAT*ST*ST*SC*SmU | | OOOSSSS | chimeric version of | |
| | | | | | | Exon51: Analog of 942 | |
| WV- | TCAAGGAAGAT | 484 | T*SC*SA*SmAG*SG*SA*SmAG*SA*S | 1236 | SSSOSSSOSSSOS | Stereopure DNA/2′OMe | Exon51 |
| 1118 | GGCAUTTCU | | T*SmGG*SC*SA*SmUT*ST*SC*SmU | | SSOSSS | chimeric version of | |
| | | | | | | Exon51: Analog of 942 | |
| WV- | UCAAGGAAGA | 485 | mUmCmAmAG*SG*SA*SA*SG*SmAm | 1237 | OOOOSSSSSOO | Stereopure DNA/2′OMe | Exon51 |
| 1119 | UGGCATTTCU | | UmGmGmCA*ST*ST*ST*SC*SmU | | OOOSSSSS | chimeric version of | |
| | | | | | | Exon51: Analog of 942 | |
| WV- | TCAAGGAAGAT | 486 | T*SC*SmAmAmGmGmAmAmGmAT*S | 1238 | SSOOOOOOOOS | Stereopure DNA/2′OMe | Exon51 |
| 1120 | GGCATTTCU | | mGmGC*SmAT*ST*ST*SC*SmU | | OOSOSSSS | chimeric version of | |
| | | | | | | Exon51: Analog of 942 | |
| WV- | TCAAGGAAGA | 487 | T*C*A*A*G*mGmAmAmGmAmUmG | 1239 | XXXXXOOOOO | Stereorandom | Exon51 |
| 1131 | UGGCATTTCT | | mGmCA*T*T*T*C*T | | OOOOXXXXX | DNA/2′OMe chimeric | |
| | | | | | | version of Exon51: | |
| | | | | | | Analog of WV1111 | |
| WV- | UCAAGGAAGA | 488 | mUmCmAmAG*G*A*mAG*A*T*G*m | 1240 | OOOOXXXOXX | Stereorandom | Exon51 |
| 1132 | TGGCATUUCU | | GC*A*T*mUmUmCmU | | XXOXXXOOO | DNA/2′OMe chimeric | |
| | | | | | | version of Exon51: | |
| | | | | | | Analog of WV1112 | |
| WV- | TCAAGGAAGAT | 489 | T*mCA*mAG*mGA*mAG*mAT*mGG | 1241 | XOXOXOXOXO | Stereorandom | Exon51 |
| 1133 | GGCAUTUCU | | *mCA*mUT*mUC*mU | | XOXOXOXOX | DNA/2′OMe chimeric | |
| | | | | | | version of Exon51: | |
| | | | | | | Analog of WV1113 | |
| WV- | UCAAGGAAGA | 490 | mUC*mAA*mGG*mAA*mGA*mUG*m | 1242 | OXOXOXOXOX | Stereorandom | Exon51 |
| 1134 | UGGCATUTCU | | GC*mAT*mUT*mCmU | | OXOXOXOXO | DNA/2′OMe chimeric | |
| | | | | | | version of Exon51: | |
| | | | | | | Analog of WV1114 | |
| WV- | TCAAGGAAGAT | 491 | T*C*mAmAG*G*mAmAG*A*T*mGm | 1243 | XXOOXXOOXX | Stereorandom | Exon51 |
| 1135 | GGCAUUTCU | | GC*A*mUmUT*C*mU | | XOOXXOOXX | DNA/2′OMe chimeric | |
| | | | | | | version of Exon51: | |
| | | | | | | Analog of WV1115 | |
| WV- | TCAAGGAAGA | 492 | T*C*A*mAmGmGA*A*mGmAmUG*G | 1244 | XXXOOOXXOO | Stereorandom | Exon51 |
| 1136 | UGGCAUTTCU | | *mCmAmUT*T*C*mU | | OXXOOOXXX | DNA/2′OMe chimeric | |
| | | | | | | version of Exon51: | |
| | | | | | | Analog of WV1116 | |
| WV- | TCAAGGAAGAT | 493 | T*C*A*A*mGmGmAmAG*A*T*mGm | 1245 | XXXXOOOOXX | Stereorandom | Exon51 |
| 1137 | GGCATTTCU | | GmCmAT*T*T*C*mU | | XOOOOXXXX | DNA/2′OMe chimeric | |
| | | | | | | version of Exon51: | |
| | | | | | | Analog of WV1117 | |
| WV- | TCAAGGAAGAT | 494 | T*C*A*mAG*G*A*mAG*A*T*mGG*C | 1246 | XXXOXXXOXX | Stereorandom | Exon51 |
| 1138 | GGCAUTTCU | | *A*mUT*T*C*mU | | XOXXXOXXX | DNA/2′OMe chimeric | |
| | | | | | | version of Exon51: | |
| | | | | | | Analog of WV1118 | |
| WV- | UCAAGGAAGA | 495 | mUmCmAmAG*G*A*A*G*mAmUmG | 1247 | OOOOXXXXXO | Stereorandom | Exon51 |
| 1139 | UGGCATTTCU | | mGmCA*T*T*T*C*mU | | OOOOXXXXX | DNA/2′OMe chimeric | |
| | | | | | | version of Exon51: | |
| | | | | | | Analog of WV1119 | |
| WV- | TCAAGGAAGAT | 496 | T*C*mAmAmGmGmAmAmGmAT*mG | 1248 | XXOOOOOOOO | Stereorandom | Exon51 |
| 1140 | GGCATTTCU | | mGC*mAT*T*T*C*mU | | XOOXOXXXX | DNA/2′OMe chimeric | |
| | | | | | | version of Exon51: | |
| | | | | | | Analog of WV1120 | |
| WV- | UCAAGGAAGA | 497 | mU*mC*mA*mA*mG*mGmAmAmGm | 1249 | XXXXXOOOOO | Stereorandom 2′OMe | Exon51 |
| 1151 | UGGCAUUUCU | | AmUmGmGmCmA*mU*mU*mU*mC* | | OOOOXXXXX | PO/PS chimeric version | |
| | | | mU | | | of exon51: Analog of | |
| | | | | | | WV1111 | |
| WV- | UCAAGGAAGA | 498 | mUmCmAmAmG*mG*mA*mAmG*mA | 1250 | OOOOXXXOXX | Stereorandom 2′OMe | Exon51 |
| 1152 | UGGCAUUUCU | | *mU*mG*mGmC*mA*mU*mUmUmCmU | | XXOXXXOOO | PO/PS chimeric version | |
| | | | | | | of exon51: Analog of | |
| | | | | | | WV1112 | |
| WV- | UCAAGGAAGA | 499 | mU*mCmA*mAmG*mGmA*mAmG*m | 1251 | XOXOXOXOXO | Stereorandom 2′OMe | Exon51 |
| 1153 | UGGCAUUUCU | | AmU*mGmG*mCmA*mUmU*mUmC* | | XOXOXOXOX | PO/PS chimeric version | |
| | | | mU | | | of exon51: Analog of | |
| | | | | | | WV1113 | |
| WV- | UCAAGGAAGA | 500 | mUmC*mAmA*mGmG*mAmA*mGmA | 1252 | OXOXOXOXOX | Stereorandom 2′OMe | Exon51 |
| 1154 | UGGCAUUUCU | | *mUmG*mGmC*mAmU*mUmU*mCmU | | OXOXOXOXO | PO/PS chimeric version | |
| | | | | | | of exon51: Analog of | |
| | | | | | | WV1114 | |
| WV- | UCAAGGAAGA | 501 | mU*mC*mAmAmG*mG*mAmAmG*m | 1253 | XXOOXXOOXX | Stereorandom 2′OMe | Exon51 |
| 1155 | UGGCAUUUCU | | A*mU*mGmGmC*mA*mUmUmU*mC* | | XOOXXOOXX | PO/PS chimeric version | |
| | | | mU | | | of exon51: Analog of | |
| | | | | | | WV1115 | |
| WV- | UCAAGGAAGA | 502 | mU*mC*mA*mAmGmGmA*mA*mGm | 1254 | XXXOOOXXOO | Stereorandom 2′OMe | Exon51 |
| 1156 | UGGCAUUUCU | | AmUmG*mG*mCmAmUmU*mU*mC* | | OXXOOOXXX | PO/PS chimeric version | |
| | | | mU | | | of exon51: Analog of | |
| | | | | | | WV1116 | |
| WV- | UCAAGGAAGA | 503 | mU*mC*mA*mA*mGmGmAmAmG*m | 1255 | XXXXOOOOXX | Stereorandom 2′OMe | Exon51 |
| 1157 | UGGCAUUUCU | | A*mU*mGmGmCmAmU*mU*mU*mC* | | XOOOOXXXX | PO/PS chimeric version | |
| | | | mU | | | of exon51: Analog of | |
| | | | | | | WV1117 | |
| WV- | UCAAGGAAGA | 504 | mU*mC*mA*mAmG*mG*mA*mAmG* | 1256 | XXXOXXXOXX | Stereorandom 2′OMe | Exon51 |
| 1158 | UGGCAUUUCU | | mA*mU*mGmG*mC*mA*mUmU*mU* | | XOXXXOXXX | PO/PS chimeric version | |
| | | | mC*mU | | | of exon51: Analog of | |
| | | | | | | WV1118 | |
| WV- | UCAAGGAAGA | 505 | mUmCmAmAmG*mG*mA*mA*mG*m | 1257 | OOOOXXXXXO | Stereorandom 2′OMe | Exon51 |
| 1159 | UGGCAUUUCU | | AmUmGmGmCmA*mU*mU*mU*mC* | | OOOOXXXXX | PO/PS chimeric version | |
| | | | mU | | | of exon51: Analog of | |
| | | | | | | WV1119 | |
| WV- | UCAAGGAAGA | 506 | mU*mC*mAmAmGmGmAmAmGmAm | 1258 | XXOOOOOOOO | Stereorandom 2′OMe | Exon51 |
| 1160 | UGGCAUUUCU | | U*mGmGmC*mAmU*mU*mU*mC*mU | | XOOXOXXXX | PO/PS chimeric version | |
| | | | | | | of exon51: Analog of | |
| | | | | | | WV1120 | |
| WV- | AGAAAUGCCA | 507 | rArGrArArArUrGrCrCrArUrCrUrUrCrCr | 1259 | OOOOOOOOOO | RNA | Exon51 |
| 1687 | UCUUCCUUGA | | UrUrGrA | | OOOOOOOOO | | |
| WV- | UCAAGGAAGA | 508 | mU*SmC*SmA*RmA*RmG*RmG*Rm | 1260 | SSRRRRRRRRRR | Exon51: 2S-15R-2S | Exon51 |
| 2363 | UGGCAUUUCU | | A*RmA*RmG*RmA*RmU*RmG*RmG | | RRRRRSS | | |
| | | | *RmC*RmA*RmU*RmU*RmU*SmC*S | | | | |
| | | | mU | | | | |
| WV- | UCAAGGAAGA | 509 | mU*SmC*SmA*SmA*SmG*RmG*RmA | 1261 | SSSSRRRRRRRR | Exon51: 4S-11R-4S | Exon51 |
| 2364 | UGGCAUUUCU | | *RmA*RmG*RmA*RmU*RmG*RmG*R | | RRRSSSS | | |
| | | | mC*RmA*RmU*SmU*SmU*SmC*SmU | | | | |
| WV- | UCAAGGAAGA | 510 | mU*SmC*SmA*SmA*SmG*SmG*RmA | 1262 | SSSSSRRRRRRR | Exon51: 5S-9R-5S | Exon51 |
| 2365 | UGGCAUUUCU | | *RmA*RmG*RmA*RmU*RmG*RmG*R | | RRSSSSS | | |
| | | | mC*RmA*SmU*SmU*SmU*SmC*SmU | | | | |
| WV- | UCAAGGAAGA | 511 | mU*SmCmAmAmGmGmAmAmGmAm | 1263 | SOOOOOOOOOO | Exon51: 1S-17PO-1S | Exon51 |
| 2366 | UGGCAUUUCU | | UmGmGmCmAmUmUmUmC*SmU | | OOOOOOOS | | |
| WV- | UCAAGGAAGA | 512 | mU*SmC*SmAmAmGmGmAmAmGmA | 1264 | SSOOOOOOOOO | Exon51: 2S-15PO-2S | Exon51 |
| 2367 | UGGCAUUUCU | | mUmGmGmCmAmUmUmU*SmC*SmU | | OOOOOOSS | | |
| WV- | UCAAGGAAGA | 513 | mU*SmC*SmA*SmAmGmGmAmAmG | 1265 | SSSOOOOOOOO | Exon51: 3S-13PO-3S | Exon51 |
| 2368 | UGGCAUUUCU | | mAmUmGmGmCmAmUmU*SmU*SmC | | OOOOOSSS | | |
| | | | *SmU | | | | |
| WV- | UCAAGGAAGA | 514 | mU*SmC*SmA*SmA*SmGmGmAmAm | 1266 | SSSSOOOOOOO | Exon51: 4S-11PO-4S | Exon51 |
| 2369 | UGGCAUUUCU | | GmAmUmGmGmCmAmU*SmU*SmU* | | OOOOSSSS | | |
| | | | SmC*SmU | | | | |
| WV- | UCAAGGAAGA | 515 | mU*SmC*SmA*SmA*SmG*SmGmAm | 1267 | SSSSSOOOOOO | Exon51: 5S-9PO-5S | Exon51 |
| 2370 | UGGCAUUUCU | | AmGmAmUmGmGmCmA*SmU*SmU* | | OOOSSSSS | | |
| | | | SmU*SmC*SmU | | | | |
| WV- | UCAAGGAAGA | 516 | mU*mCmAmAmGmGmAmAmGmAmU | 1268 | XOOOOOOOOO | Exon51: 1PS-17PO-1PS | Exon51 |
| 2381 | UGGCAUUUCU | | mGmGmCmAmUmUmUmC*mU | | OOOOOOOOX | stereorandom | |
| WV- | UCAAGGAAGA | 517 | mU*mC*mAmAmGmGmAmAmGmAm | 1269 | XXOOOOOOOO | Exon51: 2PS-15PO-2PS | Exon51 |
| 2382 | UGGCAUUUCU | | UmGmGmCmAmUmUmU*mC*mU | | OOOOOOOXX | stereorandom | |
| WV- | UCAAGGAAGA | 518 | mU*mC*mA*mAmGmGmAmAmGmA | 1270 | XXXOOOOOOO | Exon51: 3PS-13PO-3PS | Exon51 |
| 2383 | UGGCAUUUCU | | mUmGmGmCmAmUmU*mU*mC*mU | | OOOOOOXXX | stereorandom | |
| WV- | UCAAGGAAGA | 519 | mU*mC*mA*mA*mGmGmAmAmGmA | 1271 | XXXXOOOOOO | Exon51: 4PS-11PO-4PS | Exon51 |
| 2384 | UGGCAUUUCU | | mUmGmGmCmAmU*mU*mU*mC*mU | | OOOOOXXXX | stereorandom | |
| WV- | UCAAGGAAGA | 520 | mU*mC*mA*mA*mG*mGmAmAmGm | 1272 | XXXXXOOOOO | Exon51: 5PS-9PO-5PS | Exon51 |
| 2385 | UGGCAUUUCU | | AmUmGmGmCmA*mU*mU*mU*mC* | | OOOOXXXXX | stereorandom | |
| | | | mU | | | | |
| WV- | UCAAGGAAGA | 521 | fU*fC*fA*fA*fG*fG*mAmAmGmAmU | 1273 | XXXXXXOOOO | 6F-8OMe-6F 6PS-7PO- | Exon51 |
| 2432 | UGGCAUUUCU | | mGmGmC*fA*fU*fU*fU*fC*fU | | OOOXXXXXX | 6PS | |
| WV- | UCAAGGAAGA | 522 | fU*fC*fA*fA*fG*mGmAmAmGmAmU | 1274 | XXXXXOOOOO | 5F-10OMe-5F 5PS- | Exon51 |
| 2433 | UGGCAUUUCU | | mGmGmCmA*fU*fU*fU*fC*fU | | OOOOXXXXX | 9PO-5PS | |
| WV- | UCAAGGAAGA | 523 | fU*fC*fA*fA*mGmGmAmAmGmAmU | 1275 | XXXXOOOOOO | 4F-12OMe-4F 4PS- | Exon51 |
| 2434 | UGGCAUUUCU | | mGmGmCmAmU*fU*fU*fC*fU | | OOOOOXXXX | 11PO-4PS | |
| WV- | UCAAGGAAGA | 524 | fU*fC*fA*mAmGmGmAmAmGmAmU | 1276 | XXXOOOOOOO | 3F-14OMe-3F 3PS- | Exon51 |
| 2435 | UGGCAUUUCU | | mGmGmCmAmUmU*fU*fC*fU | | OOOOOOXXX | 13PO-3PS | |
| WV- | UCAAGGAAGA | 525 | fU*fC*mAmAmGmGmAmAmGmAmU | 1277 | XXOOOOOOOO | 2F-16OMe-2F 2PS- | Exon51 |
| 2436 | UGGCAUUUCU | | mGmGmCmAmUmUmU*fC*fU | | OOOOOOOXX | 15PO-2PS | |
| WV- | UCAAGGAAGA | 526 | fU*mCmAmAmGmGmAmAmGmAmU | 1278 | XOOOOOOOOO | 1F-18OMe-1F 1PS- | Exon51 |
| 2437 | UGGCAUUUCU | | mGmGmCmAmUmUmUmC*fU | | OOOOOOOOX | 17PO-1PS | |
| WV- | UCAAGGAAGA | 527 | fU*SfC*SfA*SfA*SfG*SfG*SmAmAmG | 1279 | SSSSSSOOOOOO | 6F-8OMe-6F 6Sp-7PO- | Exon51 |
| 2438 | UGGCAUUUCU | | mAmUmGmGmC*SfA*SfU*SfU*SfU*S | | OSSSSSS | 6Sp | |
| | | | fC*SfU | | | | |
| WV- | UCAAGGAAGA | 528 | fU*SfC*SfA*SfA*SfG*SmGmAmAmG | 1280 | SSSSSOOOOOO | 5F-10OMe-5F 5Sp- | Exon51 |
| 2439 | UGGCAUUUCU | | mAmUmGmGmCmA*SfU*SfU*SfU*Sf | | OOOSSSSS | 9PO-5Sp | |
| | | | C*SfU | | | | |
| WV- | UCAAGGAAGA | 529 | fU*SfC*SfA*SfA*SmGmGmAmAmGm | 1281 | SSSSOOOOOOO | 4F-12OMe-4F 4Sp- | Exon51 |
| 2440 | UGGCAUUUCU | | AmUmGmGmCmAmU*SfU*SfU*SfC*S | | OOOOSSSS | 11PO-4Sp | |
| | | | fU | | | | |
| WV- | UCAAGGAAGA | 530 | fU*SfC*SfA*SmAmGmGmAmAmGmA | 1282 | SSSOOOOOOOO | 3F-14OMe-3F 3Sp- | Exon51 |
| 2441 | UGGCAUUUCU | | mUmGmGmCmAmUmU*SfU*SfC*SfU | | OOOOOSSS | 13PO-3Sp | |
| WV- | UCAAGGAAGA | 531 | fU*SfC*SmAmAmGmGmAmAmGmAm | 1283 | SSOOOOOOOOO | 2F-16OMe-2F 2Sp- | Exon51 |
| 2442 | UGGCAUUUCU | | UmGmGmCmAmUmUmU*SfC*SfU | | OOOOOOSS | 15PO-2Sp | |
| WV- | UCAAGGAAGA | 532 | fU*SmCmAmAmGmGmAmAmGmAmU | 1284 | SOOOOOOOOOO | 1F-18OMe-1F 1Sp- | Exon51 |
| 2443 | UGGCAUUUCU | | mGmGmCmAmUmUmUmC*SfU | | OOOOOOOS | 17PO-1Sp | |
| WV- | UCAAGGAAGA | 533 | fU*SfC*SfA*SfA*SfG*SfG*SmA*RmA | 1285 | SSSSSSRRRRRR | 6F-8OMe-6F 6Sp-7Rp- | Exon51 |
| 2444 | UGGCAUUUCU | | *RmG*RmA*RmU*RmG*RmG*RmC*S | | RSSSSSS | 6Sp | |
| | | | fA*SfU*SfU*SfU*SfC*SfU | | | | |
| WV- | UCAAGGAAGA | 534 | fU*SfC*SfA*SfA*SfG*SmG*RmA*Rm | 1286 | SSSSSSRRRRRRR | 5F-10OMe-5F 5Sp-9Rp- | Exon51 |
| 2445 | UGGCAUUUCU | | A*RmG*RmA*RmU*RmG*RmG*RmC* | | RRSSSSS | 5Sp | |
| | | | RmA*SfU*SfU*SfU*SfC*SfU | | | | |
| WV- | UCAAGGAAGA | 535 | fU*SfC*SfA*SfA*SmG*RmG*RmA*Rm | 1287 | SSSSRRRRRRRR | 4F-12OMe-4F 4Sp- | Exon51 |
| 2446 | UGGCAUUUCU | | A*RmG*RmA*RmU*RmG*RmG*RmC* | | RRRSSSS | 11Rp-4Sp | |
| | | | RmA*RmU*SfU*SfU*SfC*SfU | | | | |
| WV- | UCAAGGAAGA | 536 | fU*SfC*SfA*SmA*RmG*RmG*RmA*R | 1288 | SSSRRRRRRRRR | 3F-14OMe-3F 3Sp- | Exon51 |
| 2447 | UGGCAUUUCU | | mA*RmG*RmA*RmU*RmG*RmG*Rm | | RRRRSSS | 13Rp-3Sp | |
| | | | C*RmA*RmU*RmU*SfU*SfC*SfU | | | | |
| WV- | UCAAGGAAGA | 537 | fU*SfC*SmA*RmA*RmG*RmG*RmA* | 1289 | SSRRRRRRRRRR | 2F-16OMe-2F 2Sp- | Exon51 |
| 2448 | UGGCAUUUCU | | RmA*RmG*RmA*RmU*RmG*RmG*R | | RRRRRSS | 15Rp-2Sp | |
| | | | mC*RmA*RmU*RmU*RmU*SfC*SfU | | | | |
| WV- | UCAAGGAAGA | 538 | fU*SmC*RmA*RmA*RmG*RmG*RmA | 1290 | SRRRRRRRRRR | 1F-18OMe-1F 1Sp- | Exon51 |
| 2449 | UGGCAUUUCU | | *RmA*RmG*RmA*RmU*RmG*RmG*R | | RRRRRRRS | 17Rp-1Sp | |
| | | | mC*RmA*RmU*RmU*RmU*RmC*SfU | | | | |
| WV- | UCAAGGAAGA | 539 | fU*SfC*SfA*SfA*SfG*SfG*SfA*SfA*R | 1291 | SSSSSSSRRRRRS | 7F-6OMe-7F 7Sp-5Rp- | Exon51 |
| 2526 | UGGCAUUUCU | | mG*RmA*RmU*RmG*RmG*SfC*SfA* | | SSSSSS | 7Sp | |
| | | | SfU*SfU*SfU*SfC*SfU | | | | |
| WV- | UCAAGGAAGA | 540 | fU*SfC*SfA*SfA*SfG*SfG*SfA*SfA*S | 1292 | SSSSSSSSRRRSS | 8F-4OMe-8F 8Sp-3Rp- | Exon51 |
| 2527 | UGGCAUUUCU | | mG*RmA*RmU*RmG*SfG*SfC*SfA*Sf | | SSSSSS | 8Sp | |
| | | | U*SfU*SfU*SfC*SfU | | | | |
| WV- | UCAAGGAAGA | 541 | fU*SfC*SfA*SfA*SfG*SfG*SfA*SfA*Sf | 1293 | SSSSSSSSSRSSS | 9F-2OMe-9F 9Sp-1Rp- | Exon51 |
| 2528 | UGGCAUUUCU | | G*SmA*RmU*SfG*SfG*SfC*SfA*SfU* | | SSSSSS | 9Sp | |
| | | | SfU*SfU*SfC*SfU | | | | |
| WV- | UCAAGGAAGA | 542 | fU*SfC*SfA*SfA*SfG*SfG*SfA*SfAm | 1294 | SSSSSSSOOOOO | 7F-6OMe-7F 75p-5PO- | Exon51 |
| 2529 | UGGCAUUUCU | | GmAmUmGmG*SfC*SfA*SfU*SfU*Sf | | SSSSSSS | 7Sp | |
| | | | U*SfC*SfU | | | | |
| WV- | UCAAGGAAGA | 543 | fU*SfC*SfA*SfA*SfG*SfG*SfA*SfA*S | 1295 | SSSSSSSSOOOSS | 8F-4OMe-8F 8Sp-3PO- | Exon51 |
| 2530 | UGGCAUUUCU | | mGmAmUmG*SfG*SfC*SfA*SfU*SfU* | | SSSSSS | 8Sp | |
| | | | SfU*SfC*SfU | | | | |
| WV- | UCAAGGAAGA | 544 | fU*SfC*SfA*SfA*SfG*SfG*SfA*SfA*Sf | 1296 | SSSSSSSSSOSSS | 9F-2OMe-9F 9Sp-1PO- | Exon51 |
| 2531 | UGGCAUUUCU | | G*SmAmU*SfG*SfG*SfC*SfA*SfU*Sf | | SSSSSS | 9Sp | |
| | | | U*SfU*SfC*SfU | | | | |
| WV- | UCAAGGAAGA | 545 | fU*SfC*SfA*SfA*SfG*SfG*SfA*mA*m | 1297 | SSSSSSXXXXXX | 6F-8OMe-6F 65p-7PS- | Exon51 |
| 2532 | UGGCAUUUCU | | G*mA*mU*mG*mG*fC*SfA*SfU*SfU* | | XSSSSSS | 6Sp | |
| | | | SfU*SfC*SfU | | | | |
| WV- | UCAAGGAAGA | 546 | mU*SmC*SmA*SmA*SmG*SmG*SmA | 1298 | SSSSSSRRRRRR | All-OMe 6Sp-7Rp-6Sp | Exon51 |
| 2533 | UGGCAUUUCU | | *RmA*RmG*RmA*RmU*RmG*RmG*R | | RSSSSSS | | |
| | | | mC*SmA*SmU*SmU*SmU*SmC*SmU | | | | |
| WV- | UCAAGGAAGA | 547 | mU*SmC*SmA*SmA*SmG*SmG*SmA | 1299 | SSSSSSSRRRRRS | All-OMe 7Sp-5Rp-7Sp | Exon51 |
| 2534 | UGGCAUUUCU | | *SmA*RmG*RmA*RmU*RmG*RmG*S | | SSSSSS | | |
| | | | mC*SmA*SmU*SmU*SmU*SmC*SmU | | | | |
| WV- | UCAAGGAAGA | 548 | mU*SmC*SmA*SmA*SmG*SmG*SmA | 1300 | SSSSSSSSRRRSS | All-OMe 8Sp-3Rp-8Sp | Exon51 |
| 2535 | UGGCAUUUCU | | *SmA*SmG*RmA*RmU*RmG*SmG*S | | SSSSSS | | |
| | | | mC*SmA*SmU*SmU*SmU*SmC*SmU | | | | |
| WV- | UCAAGGAAGA | 549 | mU*SmC*SmA*SmA*SmG*SmG*SmA | 1301 | SSSSSSSSSRSSS | All-OMe 9Sp-1Rp-9Sp | Exon51 |
| 2536 | UGGCAUUUCU | | *SmA*SmG*SmA*RmU*SmG*SmG*S | | SSSSSS | | |
| | | | mC*SmA*SmU*SmU*SmU*SmC*SmU | | | | |
| WV- | UCAAGGAAGA | 550 | mU*SmC*SmA*SmA*SmG*SmG*SmA | 1302 | SSSSSSXXXXXX | All-OMe 6Sp-7PS-6Sp | Exon51 |
| 2537 | UGGCAUUUCU | | *mA*mG*mA*mU*mG*mG*mC*SmA* | | XSSSSSS | | |
| | | | SmU*SmU*SmU*SmC*SmU | | | | |
| WV- | UCAAGGAAGA | 551 | L001*mU*mC*mA*mA*mG*mG*mA* | 1303 | XXXXXXXXXX | Drisapersen with C6 | Exon51 |
| 2538 | UGGCAUUUCU | | mA*mG*mA*mU*mG*mG*mC*mA*m | | XXXXXXXXXX | amino linker | |
| | | | U*mU*mU*mC*mU | | | | |
| WV- | UCAAGGAAGA | 552 | Mod013L001*mU*mC*mA*mA*mG*m | 1304 | OXXXXXXXXX | Drisapersen with C6 and | Exon51 |
| 2578 | UGGCAUUUCU | | G*mA*mA*mG*mA*mU*mG*mG*mC* | | XXXXXXXXXXX | Lauric | |
| | | | mA*mU*mU*mU*mC*mU | | | | |
| WV- | UCAAGGAAGA | 553 | Mod014L001*mU*mC*mA*mA*mG*m | 1305 | OXXXXXXXXX | Drisapersen with C6 and | Exon51 |
| 2579 | UGGCAUUUCU | | G*mA*mA*mG*mA*mU*mG*mG*mC* | | XXXXXXXXXXX | Myristic | |
| | | | mA*mU*mU*mU*mC*mU | | | | |
| WV- | UCAAGGAAGA | 554 | Mod005L001*mU*mC*mA*mA*mG*m | 1306 | OXXXXXXXXX | Drisapersen with C6 and | Exon51 |
| 2580 | UGGCAUUUCU | | G*mA*mA*mG*mA*mU*mG*mG*mC* | | XXXXXXXXXXX | Palmitic | |
| | | | mA*mU*mU*mU*mC*mU | | | | |
| WV- | UCAAGGAAGA | 555 | Mod015L001*mU*mC*mA*mA*mG*m | 1307 | OXXXXXXXXX | Drisapersen with C6 and | Exon51 |
| 2581 | UGGCAUUUCU | | G*mA*mA*mG*mA*mU*mG*mG*mC* | | XXXXXXXXXXX | Stearic | |
| | | | mA*mU*mU*mU*mC*mU | | | | |
| WV- | UCAAGGAAGA | 556 | Mod016L001*mU*mC*mA*mA*mG*m | 1308 | OXXXXXXXXX | Drisapersen with C6 and | Exon51 |
| 2582 | UGGCAUUUCU | | G*mA*mA*mG*mA*mU*mG*mG*mC* | | XXXXXXXXXXX | Oleic | |
| | | | mA*mU*mU*mU*mC*mU | | | | |
| WV- | UCAAGGAAGA | 557 | Mod017L001*mU*mC*mA*mA*mG*m | 1309 | OXXXXXXXXX | Drisapersen with C6 and | Exon51 |
| 2583 | UGGCAUUUCU | | G*mA*mA*mG*mA*mU*mG*mG*mC* | | XXXXXXXXXXX | Linoleic | |
| | | | mA*mU*mU*mU*mC*mU | | | | |
| WV- | UCAAGGAAGA | 558 | Mod018L001*mU*mC*mA*mA*mG*m | 1310 | OXXXXXXXXX | Drisapersen with C6 and | Exon51 |
| 2584 | UGGCAUUUCU | | G*mA*mA*mG*mA*mU*mG*mG*mC* | | XXXXXXXXXXX | alpha-Linolenic | |
| | | | mA*mU*mU*mU*mC*mU | | | | |
| WV- | UCAAGGAAGA | 559 | Mod019L001*mU*mC*mA*mA*mG*m | 1311 | OXXXXXXXXX | Drisapersen with C6 and | Exon51 |
| 2585 | UGGCAUUUCU | | G*mA*mA*mG*mA*mU*mG*mG*mC* | | XXXXXXXXXXX | gamma-Linolenic | |
| | | | mA*mU*mU*mU*mC*mU | | | | |
| WV- | UCAAGGAAGA | 560 | Mod006L001*mU*mC*mA*mA*mG*m | 1312 | OXXXXXXXXX | Drisapersen with C6 and | Exon51 |
| 2586 | UGGCAUUUCU | | G*mA*mA*mG*mA*mU*mG*mG*mC* | | XXXXXXXXXXX | DHA | |
| | | | mA*mU*mU*mU*mC*mU | | | | |
| WV- | UCAAGGAAGA | 561 | Mod020L001*mU*mC*mA*mA*mG*m | 1313 | OXXXXXXXXX | Drisapersen with C6 and | Exon51 |
| 2587 | UGGCAUUUCU | | G*mA*mA*mG*mA*mU*mG*mG*mC* | | XXXXXXXXXXX | Turbinaric | |
| | | | mA*mU*mU*mU*mC*mU | | | | |
| WV- | UCAAGGAAGA | 562 | Mod021*mU*mC*mA*mA*mG*mG*m | 1314 | XXXXXXXXXX | Drisapersen with C6 and | Exon51 |
| 2588 | UGGCAUUUCU | | A*mA*mG*mA*mU*mG*mG*mC*mA* | | XXXXXXXXXX | Dilinoleic | |
| | | | mU*mU*mU*mC*mU | | | | |
| WV- | UCAAGGAAGA | 563 | mU*mC*mA*mA*mG*mG*mAmAmG | 1315 | XXXXXXOOOO | All-OMe 6PS-7PO-6PS | Exon51 |
| 2660 | UGGCAUUUCU | | mAmUmGmGmC*mA*mU*mU*mU*m | | OOOXXXXXX | | |
| | | | C*mU | | | | |
| WV- | UCAAGGAAGA | 564 | mU*mC*mA*mA*mG*mG*mA*mAmG | 1316 | XXXXXXXOOO | All-OMe 7PS-5PO-7PS | Exon51 |
| 2661 | UGGCAUUUCU | | mAmUmGmG*mC*mA*mU*mU*mU* | | OOXXXXXXX | | |
| | | | mC*mU | | | | |
| WV- | UCAAGGAAGA | 565 | mU*mC*mA*mA*mG*mG*mA*mA*m | 1317 | XXXXXXXXOO | All-OMe 8PS-3PO-8PS | Exon51 |
| 2662 | UGGCAUUUCU | | GmAmUmG*mG*mC*mA*mU*mU*mU | | OXXXXXXXX | | |
| | | | *mC*mU | | | | |
| WV- | UCAAGGAAGA | 566 | mU*mC*mA*mA*mG*mG*mA*mA*m | 1318 | XXXXXXXXXO | All-OMe 9PS-1PO-9PS | Exon51 |
| 2663 | UGGCAUUUCU | | G*mAmU*mG*mG*mC*mA*mU*mU* | | XXXXXXXXX | | |
| | | | mU*mC*mU | | | | |
| WV- | UCAAGGAAGA | 567 | mU*SmC*SmA*SmA*SmG*SmG*SmA | 1319 | SSSSSSOOOOOO | All-OMe 6Sp-7PO-6Sp | Exon51 |
| 2664 | UGGCAUUUCU | | mAmGmAmUmGmGmC*SmA*SmU*S | | OSSSSSS | | |
| | | | mU*SmU*SmC*SmU | | | | |
| WV- | UCAAGGAAGA | 568 | mU*SmC*SmA*SmA*SmG*SmG*SmA | 1320 | SSSSSSSOOOOO | All-OMe 7Sp-5PO-7Sp | Exon51 |
| 2665 | UGGCAUUUCU | | *SmAmGmAmUmGmG*SmC*SmA*Sm | | SSSSSSS | | |
| | | | U*SmU*SmU*SmC*SmU | | | | |
| WV- | UCAAGGAAGA | 569 | mU*SmC*SmA*SmA*SmG*SmG*SmA | 1321 | SSSSSSSSOOOSS | All-OMe 8Sp-3PO-8Sp | Exon51 |
| 2666 | UGGCAUUUCU | | *SmA*SmGmAmUmG*SmG*SmC*Sm | | SSSSSS | | |
| | | | A*SmU*SmU*SmU*SmC*SmU | | | | |
| WV- | UCAAGGAAGA | 570 | mU*SmC*SmA*SmA*SmG*SmG*SmA | 1322 | SSSSSSSSSOSSS | All-OMe 9Sp-1PO-9Sp | Exon51 |
| 2667 | UGGCAUUUCU | | *SmA*SmG*SmAmU*SmG*SmG*SmC | | SSSSSS | | |
| | | | *SmA*SmU*SmU*SmU*SmC*SmU | | | | |
| WV- | UCAAGGAAGA | 571 | fU*fC*fA*fA*fG*fG*fA*mAmGmAmU | 1323 | XXXXXXXOOO | 7F-6OMe-7F 7PS-5PO- | Exon51 |
| 2668 | UGGCAUUUCU | | mGmG*fC*fA*fU*fU*fU*fC*fU | | OOXXXXXXX | 7PS | |
| WV- | UCAAGGAAGA | 572 | fU*fC*fA*fA*fG*fG*fA*fA*mGmAmU | 1324 | XXXXXXXXOO | 8F-4OMe-8F 8PS-3PO- | Exon51 |
| 2669 | UGGCAUUUCU | | mG*fG*fC*fA*fil*fLi*fU*fC*fU | | OXXXXXXXX | 8PS | |
| WV- | UCAAGGAAGA | 573 | fU*fC*fA*fA*fG*fG*fA*fA*fG*mAmU | 1325 | XXXXXXXXXO | 9F-2OMe-9F 9PS-1PO- | Exon51 |
| 2670 | UGGCAUUUCU | | *fG*fG*fC*fAqUqUqU*fC*fU | | XXXXXXXXX | 9PS | |
| WV- | UCAAGGAAGA | 574 | fU*SfC*SfA*SfA*SfG*SfG*SmAmAmG | 1326 | SSSSSSOOOROO | | DMD |
| 2737 | UGGCAUUUCU | | mA*RmUmGmGmC*SfA*SfU*SfU*SfU | | OSSSSSS | | |
| | | | *SfC*SfU | | | | |
| WV- | UCAAGGAAGA | 575 | fU*SfC*SfA*SfA*SfG*SfG*SmAmAmG | 1327 | SSSSSSOORRRO | | Exon 51 |
| 2738 | UGGCAUUUCU | | *RmA*RmU*RmGmGmC*SfA*SfU*Sf | | OSSSSSS | | |
| | | | U*SfU*SfC*SfU | | | | |
| WV- | UCAAGGAAGA | 576 | fU*SfC*SfA*SfA*SfG*SfG*SmAmA*R | 1328 | SSSSSSORRRRR | | Exon 51 |
| 2739 | UGGCAUUUCU | | mG*RmA*RmU*RmG*RmGmC*SfA*Sf | | OSSSSSS | | |
| | | | U*SfU*SfU*SfC*SfU | | | | |
| WV- | UCAAGGAAGA | 577 | fU*SfC*SfA*SfA*SfG*SfG*SmA*RmA | 1329 | SSSSSSRROOOR | | Exon 51 |
| 2740 | UGGCAUUUCU | | *RmGmAmUmG*RmG*RmC*SfA*SfU* | | RSSSSSS | | |
| | | | SfU*SfU*SfC*SfU | | | | |
| WV- | UCAAGGAAGA | 578 | fU*SfC*SfA*SfA*SfG*SfG*SmA*RmA | 1330 | SSSSSSROOOOO | | Exon 51 |
| 2741 | UGGCAUUUCU | | mGmAmUmGmG*RmC*SfA*SfU*SfU* | | RSSSSSS | | |
| | | | SfU*SfC*SfU | | | | |
| WV- | UCAAGGAAGA | 579 | fU*SfC*SfA*SfA*SfG*SfG*SmA*SmA* | 1331 | SSSSSSSSOOOSS | | Exon 51 |
| 2742 | UGGCAUUUCU | | SmGmAmUmG*SmG*SmC*SfA*SfU*S | | SSSSSS | | |
| | | | fU*SfU*SfC*SfU | | | | |
| WV- | UCAAGGAAGA | 580 | fU*SfC*SfA*SfA*SfG*SfG*SmA*SmA | 1332 | SSSSSSSOOOOO | | Exon 51 |
| 2743 | UGGCAUUUCU | | mGmAmUmGmG*SmC*SfA*SfU*SfU* | | SSSSSSS | | |
| | | | SfU*SfC*SfU | | | | |
| WV- | UCAAGGAAGA | 581 | fU*SfC*SfA*SfA*SfG*SfG*SmA*SmA* | 1333 | SSSSSSSSSSSSSS | | Exon 51 |
| 2744 | UGGCAUUUCU | | SmG*SmA*SmU*SmG*SmG*SmC*SfA | | SSSSS | | |
| | | | *SfU*SfU*SfU*SfC*SfU | | | | |
| WV- | UCAAGGAAGA | 582 | fU*SfC*SfA*SfA*SfG*SfG*SmAmAmG | 1334 | SSSSSSOOOOSO | | Exon 51 |
| 2745 | UGGCAUUUCU | | mAfU*SmGmG*SfC*SfA*SfU*SfU*SfU | | SSSSSSS | | |
| | | | *SfC*SfU | | | | |
| WV- | UCAAGGAAGA | 583 | fU*SfC*SfA*SfA*SfG*SfG*SmA*RmA | 1335 | SSSSSSRRRRSRS | | Exon 51 |
| 2746 | UGGCAUUUCU | | *RmG*RmA*RfU*SmG*RmG*SfC*SfA | | SSSSSS | | |
| | | | *SfU*SfU*SfU*SfC*SfU | | | | |
| WV- | UCAAGGAAGA | 584 | fU*SfC*SfA*SfA*SmG*SmG*SfAfAmG | 1336 | SSSSSSOOOOSO | | Exon 51 |
| 2747 | UGGCAUUUCU | | mAfU*SmGmG*SfC*SfA*SfU*SfU*SfU | | SSSSSSS | | |
| | | | *SfC*SfU | | | | |
| WV- | UCAAGGAAGA | 585 | fU*SfC*SfA*SfA*SmG*SmG*SfA*RfA | 1337 | SSSSSSRRRRSRS | | Exon 51 |
| 2748 | UGGCAUUUCU | | *RmG*RmA*RfU*SmG*RmG*SfC*SfA | | SSSSSS | | |
| | | | *SfU*SfU*SfU*SfC*SfU | | | | |
| WV- | UCAAGGAAGA | 586 | fU*SfC*SfA*SfA*SfG*SfG*SfA*SmAm | 1338 | SSSSSSSOOOOO | | Exon 51 |
| 2749 | UGGCAUUUCU | | GmAmUmGmG*SfC*SfA*SfU*SfU*Sf | | SSSSSSS | | |
| | | | U*SfC*SfU | | | | |
| WV- | UCAAGGAAGA | 587 | fU*SfC*SfA*SfA*SfG*SfG*SfA*SmA* | 1339 | SSSSSSSRRRRRS | | Exon 51 |
| 2750 | UGGCAUUUCU | | RmG*RmA*RmU*RmG*RmG*SfC*SfA | | SSSSSS | | |
| | | | *SfU*SfU*SfU*SfC*SfU | | | | |
| WV- | UCAAGGAAGA | 588 | mU*SmC*SmA*SfA*SfG*SfG*SmA*R | 1340 | SSSSSSRRRRRR | | Exon 51 |
| 2791 | UGGCAUUUCU | | mA*RmG*RmA*RmU*RmG*RmG*Rm | | RSSSSSS | | |
| | | | C*SfA*SfU*SfU*SmU*SmC*SmU | | | | |
| WV- | UCAAGGAAGA | 589 | mU*SmC*SmA*SfA*SfG*SfG*SfA*Sm | 1341 | SSSSSSSRRRRRS | | Exon 51 |
| 2792 | UGGCAUUUCU | | A*RmG*RmA*RmU*RmG*RmG*SfC*S | | SSSSSS | | |
| | | | fA*SfU*SfU*SmU*SmC*SmU | | | | |
| WV- | UCAAGGAAGA | 590 | mU*SmC*SmA*SfA*SfG*SfG*SfA*SfA | 1342 | SSSSSSSSRRRSS | | Exon 51 |
| 2793 | UGGCAUUUCU | | *SmG*RmA*RmU*RmG*SfG*SfC*SfA | | SSSSSS | | |
| | | | *SfU*SfU*SmU*SmC*SmU | | | | |
| WV- | UCAAGGAAGA | 591 | mU*SmC*SmA*SfA*SfG*SfG*SfA*SfA | 1343 | SSSSSSSSSRSSS | | Exon 51 |
| 2794 | UGGCAUUUCU | | *SfG*SmA*RmU*SfG*SfG*SfC*SfA*Sf | | SSSSSS | | |
| | | | U*SfU*SmU*SmC*SmU | | | | |
| WV- | UCAAGGAAGA | 592 | mU*SmC*SmA*SfA*SfG*SfG*SfA*SfA | 1344 | SSSSSSSSOOOSS | | Exon 51 |
| 2795 | UGGCAUUUCU | | *SmGmAmUmG*SfG*SfC*SfA*SfU*Sf | | SSSSSS | | |
| | | | U*SmU*SmC*SmU | | | | |
| WV- | UCAAGGAAGA | 593 | mU*SmC*SmA*SfA*SfG*SfG*SfA*SfA | 1345 | SSSSSSSSSOSSS | | Exon 51 |
| 2796 | UGGCAUUUCU | | *SfG*SmAmU*SfG*SfG*SfC*SfA*SfU* | | SSSSSS | | |
| | | | SfU*SmU*SmC*SmU | | | | |
| WV- | UCAAGGAAGA | 594 | fU*fC*fA*fA*fG*fG*fA*fA*mG*mA*m | 1346 | XXXXXXXXXX | randomer based on WV- | DMD |
| 2797 | UGGCAUUUCU | | U*mG*mG*fC*fA*fU*fU*fU*fC*fU | | XXXXXXXXX | 2526 | |
| WV- | UCAAGGAAGA | 595 | fU*fC*fA*fA*fG*fG*fA*fA*mG*mA*m | 1347 | XXXXXXXXXX | randomer based on WV- | DMD |
| 2798 | UGGCAUUUCU | | U*mG*fG*fC*fA*fU*fU*fU*fC*fU | | XXXXXXXXX | 2527 | |
| WV- | UCAAGGAAGA | 596 | fU*fC*fA*fA*fG*fG*fA*fA*fG*mA*m | 1348 | XXXXXXXXXX | randomer based on WV- | DMD |
| 2799 | UGGCAUUUCU | | U*fG*fG*fC*fA*fU*fU*fU*fC*fU | | XXXXXXXXX | 2528 | |
| WV- | UCAAGGAAGA | 597 | fU*fC*fA*fA*fG*fG*fA*mA*mG*mA* | 1349 | XXXXXXXXXX | randomer based on WV- | DMD |
| 2800 | UGGCAUUUCU | | mU*mG*mG*fC*fA*fU*fU*fU*fC*fU | | XXXXXXXXX | 2750 | |
| WV- | UCAAGGAAGA | 598 | mU*mC*mA*fA*fG*fG*mA*mA*mG* | 1350 | XXXXXXXXXX | randomer based on WV- | DMD |
| 2801 | UGGCAUUUCU | | mA*mU*mG*mG*mC*fA*fU*fU*mU* | | XXXXXXXXX | 2791 | |
| | | | mC*mU | | | | |
| WV- | UCAAGGAAGA | 599 | mU*mC*mA*fA*fG*fG*fA*mA*mG*m | 1351 | XXXXXXXXXX | randomer based on WV- | DMD |
| 2802 | UGGCAUUUCU | | A*mU*mG*mG*fC*fA*fU*fU*mU*mC | | XXXXXXXXX | 2792 | |
| | | | *mU | | | | |
| WV- | UCAAGGAAGA | 600 | mU*mC*mA*fA*fG*fG*fA*fA*mG*m | 1352 | XXXXXXXXXX | randomer based on WV- | DMD |
| 2803 | UGGCAUUUCU | | A*mU*mG*fG*fC*fA*fU*fU*mU*mC* | | XXXXXXXXX | 2793 | |
| | | | mU | | | | |
| WV- | UCAAGGAAGA | 601 | mU*mC*mA*fA*fG*fG*fA*fA*fG*mA | 1353 | XXXXXXXXXX | randomer based on WV- | DMD |
| 2804 | UGGCAUUUCU | | *mU*fG*fG*fC*fA*fU*fU*mU*mC*mU | | XXXXXXXXX | 2794 | |
| WV- | UCAAGGAAGA | 602 | mU*mC*mA*fA*fG*fG*fA*fA*mGmA | 1354 | XXXXXXXXOO | randomer based on WV- | DMD |
| 2805 | UGGCAUUUCU | | mUmG*fG*fC*fA*fU*fU*mU*mC*mU | | OXXXXXXXX | 2795 | |
| WV- | UCAAGGAAGA | 603 | mU*mC*mA*fA*fG*fG*fA*fA*fG*mA | 1355 | XXXXXXXXXO | randomer based on WV- | DMD |
| 2806 | UGGCAUUUCU | | mU*fG*fG*fC*fA*fU*fU*mU*mC*mU | | XXXXXXXXX | 2796 | |
| WV- | UCAAGGAAGA | 604 | Mod024L001*mU*mC*mA*mA*mG*m | 1356 | XXXXXXXXXX | All-OMe full-PS | Exon 51 |
| 2807 | UGGCAUUUCU | | G*mA*mA*mG*mA*mU*mG*mG*mC* | | XXXXXXXXXX | TriGlcNAc conjugated | |
| | | | mA*mU*mU*mU*mC*mU | | | WV942 C6 PS | |
| WV- | UCAAGGAAGA | 605 | Mod026L001*mU*mC*mA*mA*mG*m | 1357 | XXXXXXXXXX | All-OMe full-PS | Exon 51 |
| 2808 | UGGCAUUUCU | | G*mA*mA*mG*mA*mU*mG*mG*mC* | | XXXXXXXXXX | TrialphaMannose | |
| | | | mA*mU*mU*mU*mC*mU | | | conjugated WV942 C6 | |
| | | | | | | PS | |
| WV- | UCAAGGAAGA | 606 | fU*fC*fA*fA*fG*fG*mA*mA*mG*mA | 1358 | XXXXXXXXXX | WV-1714 based BrdU in | DMD |
| 2812 | TGGCAUUUCU | | *BrdU*mG*mG*mC*fA*fU*fU*fU*fC* | | XXXXXXXXX | the center | exon 51 |
| | | | fU | | | | |
| WV- | UCAAGGAAGA | 607 | fU*fC*fA*fA*fG*fG*fA*fA*fG*mA*Br | 1359 | XXXXXXXXXX | WV-2528 and WV-2799 | DMD |
| 2813 | TGGCAUUUCU | | dU*fG*fG*fC*fA*fU*fU*fU*fC*fU | | XXXXXXXXX | based randomer BrdU in | exon 51 |
| | | | | | | the center | |
| WV- | UCAAGGAAGA | 608 | mU*mC*mA*mA*mG*mG*mA*mA*m | 1360 | XXXXXXXXXX | WV-942 based BrdU in | DMD |
| 2814 | TGGCAUUUCU | | G*mA*BrdU*mG*mG*mC*mA*mU*m | | XXXXXXXXX | the center | exon 51 |
| | | | U*mU*mC*mU | | | | |
| WV- | UCAAGGAAGA | 609 | fU*SfC*SfA*SfA*SfG*SfG*SfA*SfA*S | 1361 | SSSSSSSSOOOSS | WV-2530 based, BrdU | Exon 51 |
| 3017 | TGGCAUUUCU | | mGmABrdUmG*SfG*SfC*SfA*SfU*Sf | | SSSSSS | in the middle | |
| | | | U*SfU*SfC*SfU | | | | |
| WV- | UCAAGGAAGA | 610 | fU*fC*fA*fA*fG*fG*fA*fA*mGmABrd | 1362 | XXXXXXXXOO | WV-2530 based, | Exon 51 |
| 3018 | TGGCAUUUCU | | UmG*fG*fC*fA*fU*fU*fU*fC*fU | | OXXXXXXXX | randomer, BrdU in the | |
| | | | | | | middle | |
| WV- | UCAAGGAAGA | 611 | fU*SfC*SfA*SfA*SfG*SfG*SmAmAmG | 1363 | SSSSSSOOOOOO | WV-2438 based, BrdU | Exon 51 |
| 3019 | TGGCAUUUCU | | mABrdUmGmGmC*SfA*SfU*SfU*SfU* | | OSSSSSS | in the middle | |
| | | | SfC*SfU | | | | |
| WV- | UCAAGGAAGA | 612 | fU*fC*fA*fA*fG*fG*mAmAmGmABrd | 1364 | XXXXXXOOOO | WV-2438 based, | Exon 51 |
| 3020 | TGGCAUUUCU | | UmGmGmC*fA*fU*fU*fU*fC*fU | | OOOXXXXXX | randomer, BrdU in the | |
| | | | | | | middle | |
| WV- | UCAAGGAAGA | 613 | L001*fU*SfC*SfA*SfA*SfG*SfG*SmA | 1365 | XSSSSSSOOOOO | WV-2438 based; C6 PS; | DMD |
| 3022 | UGGCAUUUCU | | mAmGmAmUmGmGmC*SfA*SfU*SfU | | OOSSSSSS | on support; used for | |
| | | | *SfU*SfC*SfU | | | conjugation | |
| WV- | UCAAGGAAGA | 614 | Mod015L001*fU*SfC*SfA*SfA*SfG*Sf | 1366 | OXSSSSSSOOOO | WV-2438 based; | DMD |
| 3023 | UGGCAUUUCU | | G*SmAmAmGmAmUmGmGmC*SfA*S | | OOOSSSSSS | conjugate with stearic | |
| | | | fU*SfU*SfU*SfC*SfU | | | acid C6 PS | |
| WV- | UCAAGGAAGA | 615 | Mod006L001*fU*SfC*SfA*SfA*SfG*Sf | 1367 | OXSSSSSSOOOO | WV-2438 based; | DMD |
| 3024 | UGGCAUUUCU | | G*SmAmAmGmAmUmGmGmC*SfA*S | | OOOSSSSSS | conjugate with DHA C6 | |
| | | | fU*SfU*SfU*SfC*SfU | | | PS | |
| WV- | UCAAGGAAGA | 616 | L001*fU*SfC*SfA*SfA*SfG*SfG*SfA* | 1368 | XSSSSSSSSOOO | WV-2530 based; C6 PS; | DMD |
| 3025 | UGGCAUUUCU | | SfA*SmGmAmUmG*SfG*SfC*SfA*SfU | | SSSSSSSS | on support; used for | |
| | | | *SfU*SfU*SfC*SfU | | | conjugation | |
| WV- | UCAAGGAAGA | 617 | Mod015L001*fU*SfC*SfA*SfA*SfG*Sf | 1369 | OXSSSSSSSSOO | WV-2530 based; | DMD |
| 3026 | UGGCAUUUCU | | G*SfA*SfA*SmGmAmUmG*SfG*SfC* | | OSSSSSSSS | conjugate with stearic | |
| | | | SfA*SfU*SfU*SfU*SfC*SfU | | | acid C6 PS | |
| WV- | UCAAGGAAGA | 618 | Mod006L001*fU*SfC*SfA*SfA*SfG*Sf | 1370 | OXSSSSSSSSOO | WV-2530 based; | DMD |
| 3027 | UGGCAUUUCU | | G*SfA*SfA*SmGmAmUmG*SfG*SfC* | | OSSSSSSSS | conjugate with DHA C6 | |
| | | | SfA*SfU*SfU*SfU*SfC*SfU | | | PS | |
| WV- | UCAAGGAAGA | 619 | fU*SfC*SfA*SfA*SfG*SfG*SfA*SfA*S | 1371 | SSSSSSSSOOOO | WV-2529 based, convert | DMD |
| 3028 | UGGCAUUUCU | | mGmAmUmGmG*SfC*SfA*SfU*SfU*S | | SSSSSSS | PO between 8th and 9th | |
| | | | fU*SfC*SfU | | | nt to PS | |
| WV- | UCAAGGAAGA | 620 | L001*fU*fC*fA*fA*fG*fG*mA*mA*m | 1372 | XXXXXXXXXX | WV-1714 based; | DMD |
| 3029 | UGGCAUUUCU | | G*mA*mU*mG*mG*mC*fA*fU*fU*fU | | XXXXXXXXXX | stereorandom; C6 PS; on | |
| | | | *fC*fU | | | support | |
| WV- | UCAAGGAAGA | 621 | Mod015L001*fU*fC*fA*fA*fG*fG*mA | 1373 | OXXXXXXXXX | WV-1714 based; | DMD |
| 3030 | UGGCAUUUCU | | *mA*mG*mA*mU*mG*mG*mC*fA*fU | | XXXXXXXXXXX | stereorandom; conjugate | |
| | | | *fU*fU*fC*fU | | | with stearic acid C6 PS | |
| WV- | UCAAGGAAGA | 622 | Mod006L001*f1J*fC*fA*fA*fG*fG*mA | 1374 | OXXXXXXXXX | WV-1714 based; | DMD |
| 3031 | UGGCAUUUCU | | *mA*mG*mA*mU*mG*mG*mC*fA*fU | | XXXXXXXXXXX | stereorandom; conjugate | |
| | | | *fU*fU*fC*fU | | | with DHA C6 PS | |
| WV- | UCAAGGAAGA | 623 | Mod020L001*fU*fC*fA*fA*fG*fG*mA | 1375 | OXXXXXXXXX | WV-1714 based; | DMD |
| 3032 | UGGCAUUUCU | | *mA*mG*mA*mU*mG*mG*mC*fA*fU | | XXXXXXXXXXX | stereorandom; conjugate | |
| | | | *fU*fU*fC*fU | | | with turbinaric acid C6 | |
| | | | | | | PS | |
| WV- | UCAAGGAAGA | 624 | Mod019L001*fU*fC*fA*fA*fG*fG*mA | 1376 | OXXXXXXXXX | WV-1714 based; | DMD |
| 3033 | UGGCAUUUCU | | *mA*mG*mA*mU*mG*mG*mC*fA*fU | | XXXXXXXXXXX | stereorandom; conjugate | |
| | | | *fU*fU*fC*fU | | | with gamma-Linolenic | |
| | | | | | | acid C6 PS | |
| WV- | UCAAGGAAGA | 625 | L001*fU*fC*fA*fA*fG*fG*fA*fA*mG | 1377 | XXXXXXXXXO | WV-2530 based; | DMD |
| 3034 | UGGCAUUUCU | | mAmUmG*fG*fC*fA*fU*fU*fU*fC*fU | | OOXXXXXXXX | stereorandom; C6 PS; on | |
| | | | | | | support | |
| WV- | UCAAGGAAGA | 626 | Mod015L001*fU*fC*fA*fA*fG*fG*fA*f | 1378 | OXXXXXXXXX | WV-2530 based; | DMD |
| 3035 | UGGCAUUUCU | | A*mGmAmUmG*fG*fC*fA*fU*fU*fU* | | OOOXXXXXXXX | stereorandom; conjugate | |
| | | | fC*fU | | | with stearic acid C6 PS | |
| WV- | UCAAGGAAGA | 627 | Mod006L001*fU*fC*fA*fA*fG*fG*fA*f | 1379 | OXXXXXXXXX | WV-2530 based; | DMD |
| 3036 | UGGCAUUUCU | | A*mGmAmUmG*fG*fC*fA*fU*fU*fU* | | OOOXXXXXXXX | stereorandom; conjugate | |
| | | | fC*fU | | | with DHA C6 PS | |
| WV- | UCAAGGAAGA | 628 | Mod020L001*fU*fC*fA*fA*fG*fG*fA*f | 1380 | OXXXXXXXXX | WV-2530 based; | DMD |
| 3037 | UGGCAUUUCU | | A*mGmAmUmG*fG*fC*fA*fU*fU*fU* | | OOOXXXXXXXX | stereorandom; conjugate | |
| | | | fC*fU | | | with turbinaric acid C6 | |
| | | | | | | PS | |
| WV- | UCAAGGAAGA | 629 | Mod019L001*fU*fC*fA*fA*fG*fG*fA*f | 1381 | OXXXXXXXXX | WV-2530 based; | DMD |
| 3038 | UGGCAUUUCU | | A*mGmAmUmG*fG*fC*fA*fU*fU*fU* | | OOOXXXXXXXX | stereorandom; conjugate | |
| | | | fC*fU | | | with gamma-Linolenic | |
| | | | | | | acid C6 PS | |
| WV- | UCAAGGAAGA | 630 | fU*fC*fA*fA*fG*fG*mAmAmGmA*m | 1382 | XXXXXXOOOX | Randomer of WV-2737; | DMD |
| 3039 | UGGCAUUUCU | | UmGmGmC*fA*fU*fU*fU*fC*fU | | OOOXXXXXX | based on WV-2438; with | exon 51 |
| | | | | | | Rp/PO in the core | |
| WV- | UCAAGGAAGA | 631 | fU*fC*fA*fA*fG*fG*mAmAmG*mA*m | 1383 | XXXXXXOOXX | Randomer of WV-2738; | DMD |
| 3040 | UGGCAUUUCU | | U*mGmGmC*fA*fU*fU*fU*fC*fU | | XOOXXXXXX | based on WV-2438; with | exon 51 |
| | | | | | | Rp/PO in the core | |
| WV- | UCAAGGAAGA | 632 | fU*fC*fA*fA*fG*fG*mAmA*mG*mA* | 1384 | XXXXXXOXXX | Randomer of WV-2739; | DMD |
| 3041 | UGGCAUUUCU | | mU*mG*mGmC*fA*fU*fU*fU*fC*fU | | XXOXXXXXX | based on WV-2438; with | exon 51 |
| | | | | | | Rp/PO in the core | |
| WV- | UCAAGGAAGA | 633 | fU*fC*fA*fA*fG*fG*mA*mA*mGmAm | 1385 | XXXXXXXXOO | Randomer of WV-2740; | DMD |
| 3042 | UGGCAUUUCU | | UmG*mG*mC*fA*fU*fU*fU*fC*fU | | OXXXXXXXX | based on WV-2438; with | exon 51 |
| | | | | | | Rp/PO in the core | |
| WV- | UCAAGGAAGA | 634 | fU*fC*fA*fA*fG*fG*mA*mAmGmAm | 1386 | XXXXXXXOOO | Randomer of WV-2741; | DMD |
| 3043 | UGGCAUUUCU | | UmGmG*mC*fA*fU*fU*fU*fC*fU | | OOXXXXXXX | based on WV-2438; with | exon 51 |
| | | | | | | Rp/PO in the core | |
| WV- | UCAAGGAAGA | 635 | fU*fC*fA*fA*fG*fG*mA*mA*mGmAm | 1387 | XXXXXXXXOO | Randomer of WV-2742; | DMD |
| 3044 | UGGCAUUUCU | | UmG*mG*mC*fA*fU*fU*fU*fC*fU | | OXXXXXXXX | based on WV-2438; with | exon 51 |
| | | | | | | Sp/PO in the core | |
| WV- | UCAAGGAAGA | 636 | fU*fC*fA*fA*fG*fG*mA*mAmGmAm | 1388 | XXXXXXXOOO | Randomer of WV-2743; | DMD |
| 3045 | UGGCAUUUCU | | UmGmG*mC*fA*fU*fU*fU*fC*fU | | OOXXXXXXX | based on WV-2438; with | exon 51 |
| | | | | | | Sp/PO in the core | |
| WV- | UCAAGGAAGA | 637 | fU*fC*fA*fA*fG*fG*mAmAmGmAfU* | 1389 | XXXXXXOOOO | Randomer of WV-2745; | DMD |
| 3046 | UGGCAUUUCU | | mGmG*fC*fA*fU*fU*fU*fC*fU | | XOXXXXXXX | based on WV-2444; | exon 51 |
| | | | | | | Sp/PO in the core; with | |
| | | | | | | additional fU fC in the | |
| | | | | | | core | |
| WV- | UCAAGGAAGA | 638 | fU*fC*fA*fA*fG*fG*mA*mA*mG*mA | 1390 | XXXXXXXXXX | Randomer of WV-2746; | DMD |
| 3047 | UGGCAUUUCU | | *fU*mG*mG*fC*fA*fU*fU*fU*fC*fU | | XXXXXXXXX | based on WV-2444; | exon 51 |
| | | | | | | Sp/Rp in the core; with | |
| | | | | | | additional fU fC in the | |
| | | | | | | core | |
| WV- | UCAAGGAAGA | 639 | fU*fC*fA*fA*mG*mG*fAfAmGmAfU* | 1391 | XXXXXXOOOO | Randomer of WV-2747; | DMD |
| 3048 | UGGCAUUUCU | | mGmG*fC*fA*fU*fU*fU*fC*fU | | XOXXXXXXX | based on WV-2444; | exon 51 |
| | | | | | | Sp/PO in the core; with | |
| | | | | | | mGmG on left wing, | |
| | | | | | | with additional fA fA fU | |
| | | | | | | fC in the core | |
| WV- | UCAAGGAAGA | 640 | fU*fC*fA*fA*mG*mG*fA*fA*mG*mA | 1392 | XXXXXXXXXX | Randomer of WV-2748; | DMD |
| 3049 | UGGCAUUUCU | | *fU*mG*mG*fC*fA*fU*fU*fU*fC*fU | | XXXXXXXXX | based on WV-2444; | exon 51 |
| | | | | | | Sp/Rp in the core; with | |
| | | | | | | mGmG on left wing, | |
| | | | | | | with additional fA fA fU | |
| | | | | | | fC in the core | |
| WV- | UCAAGGAAGA | 641 | fU*fC*fA*fA*fG*fG*mA*mA*mG*mA | 1393 | XXXXXXXXXX | All PS version of the | DMD |
| 3050 | UGGCAUUUCU | | *fU*mG*mG*fC*fA*fU*fU*fU*fC*fU | | XXXXXXXXX | randomer of WV- | exon 51 |
| | | | | | | 2745/2746; based on | |
| | | | | | | WV-2444; with | |
| | | | | | | additional fU fC in the | |
| | | | | | | core | |
| WV- | UCAAGGAAGA | 642 | fU*fC*fA*fA*mG*mG*fA*fA*mG*mA | 1394 | XXXXXXXXXX | All PS version of the | DMD |
| 3051 | UGGCAUUUCU | | *fU*mG*mG*fC*fA*fU*fU*fU*fC*fU | | XXXXXXXXX | randomer of WV- | exon 51 |
| | | | | | | 2747/2748; based on | |
| | | | | | | WV-2444; Sp/PO in the | |
| | | | | | | core; with mGmG on | |
| | | | | | | left wing, with | |
| | | | | | | additional fA fA fU fC | |
| | | | | | | in the core | |
| WV- | UCAAGGAAGA | 643 | fU*fC*fA*fA*mG*mG*fA*fA*mGmAm | 1395 | XXXXXXXXOO | Based on WV-2530; | DMD |
| 3052 | UGGCAUUUCU | | UmG*mG*fC*fA*fU*fU*fU*fC*fU | | OXXXXXXXX | replace all 2′F G with | exon 51 |
| | | | | | | 2′Ome G | |
| WV- | UCAAGGAAGA | 644 | fU*fC*fA*fA*mG*mG*mA*mA*mGmA | 1396 | XXXXXXXXOO | Based on WV-2107; | DMD |
| 3053 | UGGCAUUUCU | | fUmG*mG*fC*fA*fU*fU*fU*fC*fU | | OXXXXXXXX | four 2′-F on the 5′; seven | exon 51 |
| | | | | | | 2′-F on the 3′; 2′F U in | |
| | | | | | | the center | |
| WV- | UCAAGGAAGA | 645 | fU*fC*fA*fA*mG*mG*fA*fA*mG*mA | 1397 | XXXXXXXXXX | All PS; based on WV- | DMD |
| 3054 | UGGCAUUUCU | | *mU*mG*mG*fC*fA*fU*fU*fU*fC*fU | | XXXXXXXXX | 2530/2529; replace all | exon 51 |
| | | | | | | 2′F G with 2′Ome G | |
| WV- | UCAAGGAAGA | 646 | fU*fC*fA*fA*mG*mG*mA*mA*mG*m | 1398 | XXXXXXXXXX | All PS; based on WV- | DMD |
| 3055 | UGGCAUUUCU | | A*fU*mG*mG*fC*fA*fU*fU*fU*fC*fU | | XXXXXXXXX | 2107; four 2′-F on the 5′; | exon 51 |
| | | | | | | seven 2′-F on the 3′; 2′F | |
| | | | | | | U in the center | |
| WV- | UCAAGGAAGA | 647 | fU*fC*fA*fA*mG*mG*fAfAmGmA*fU | 1399 | XXXXXXOOOX | Based on WV-2747; | DMD |
| 3056 | UGGCAUUUCU | | *mGmG*fC*fA*fU*fU*fU*fC*fU | | XOXXXXXXX | with additional PS in the | exon 51 |
| | | | | | | center between A and U | |
| WV- | UCAAGGAAGA | 648 | fU*fC*fA*fA*mG*mG*fA*fA*mG*mA | 1400 | XXXXXXXXXX | All PS version; based on | DMD |
| 3057 | UGGCAUUUCU | | *fU*mG*mG*fC*fA*fU*fU*fU*fC*fU | | XXXXXXXXX | WV-2747; with | exon 51 |
| | | | | | | additional PS in the | |
| | | | | | | center between A and U | |
| WV- | UCAAGGAAGA | 649 | fU*fC*fA*fA*mG*mG*fA*fA*mG*fA*f | 1401 | XXXXXXXXXX | Based on WV-1716; | DMD |
| 3058 | UGGCAUUUCU | | U*mG*mG*fC*fA*fU*fU*fU*fC*fU | | XXXXXXXXX | with all mC converted to | exon 51 |
| | | | | | | fC | |
| WV- | UCAAGGAAGA | 650 | fU*fC*fA*fA*mG*mG*fA*fA*mGmAm | 1402 | XXXXXXXXOO | Randomers of based on | DMD |
| 3059 | UGGCAUUUCU | | UmGmG*fC*fA*fU*fU*fU*fC*fU | | OOXXXXXXX | WV-2529; with all G as | exon 51 |
| | | | | | | mG; with additional PS | |
| | | | | | | between A and G | |
| WV- | UCAAGGAAGA | 651 | fU*fC*fA*fA*mG*mG*fA*fA*mGmAf | 1403 | XXXXXXXXOO | Randomer; Sp/PO in the | DMD |
| 3060 | UGGCAUUUCU | | U*mGmG*fC*fA*fU*fU*fU*fC*fU | | XOXXXXXXX | core; with mGmG on | exon 51 |
| | | | | | | left wing, with | |
| | | | | | | additional fA fA fU fC | |
| | | | | | | in the core | |
| WV- | UCAAGGAAGA | 652 | fU*fC*fA*fA*mG*mG*mA*mA*mGmA | 1404 | XXXXXXXXOO | Based on WV-2107; | DMD |
| 3061 | UGGCAUUUCU | | fU*mGmG*fC*fA*fU*fU*fU*fC*fU | | XOXXXXXXX | four 2′-F on the 5′; seven | exon 51 |
| | | | | | | 2′-F on the 3′; 2′F U in | |
| | | | | | | the center | |
| WV- | UCAAGGAAGA | 653 | fU*SfC*SfA*SfA*SfG*SfG*SmAmAmG | 1405 | SSSSSSOOOODO | WV-2438 based, with | Exon 51 |
| 3070 | UGGCAUUUCU | | mAmU:mGmGmC*SfA*SfU*SfU*SfU* | | OSSSSSS | PS2 after nucleotide 11 | |
| | | | SfC*SfU | | | | |
| WV- | UCAAGGAAGA | 654 | fU*SfC*SfA*SfA*SfG*SfG*SmAmA:m | 1406 | SSSSSSODODOD | WV-2438 based, with | Exon 51 |
| 3071 | UGGCAUUUCU | | GmA:mUmG:mGmC*SfA*SfU*SfU*SfU | | OSSSSSS | PS2 after nucleotide 8, | |
| | | | *SfC*SfU | | | 10, 12 | |
| WV- | UCAAGGAAGA | 655 | fU*SfC*SfA*SfA*SfG*SfG*SmA:mAm | 1407 | SSSSSSDODODO | WV-2438 based, with | Exon 51 |
| 3072 | UGGCAUUUCU | | G:mAmU:mGmG:mC*SfA*SfU*SfU*Sf | | DSSSSSS | PS2 after nucleotide 7, | |
| | | | U*SfC*SfU | | | 9, 11, 13 | |
| WV- | UCAAGGAAGA | 656 | fU*SfC*SfA*SfA*SfG*SfG*SmA:mAm | 1408 | SSSSSSDOOODO | WV-2438 based, with | Exon 51 |
| 3073 | UGGCAUUUCU | | GmAmU:mGmG:mC*SfA*SfU*SfU*SfU | | DSSSSSS | PS2 after nucleotide 7, | |
| | | | *SfC*SfU | | | 10, 13 | |
| WV- | UCAAGGAAGA | 657 | fU*SfC*SfA*SfA*fGSG:mAmAmGmA | 1409 | SSSXDDOOOOD | WV-2438 based, with | Exon 51 |
| 3074 | UGGCAUUUCU | | mU:mGmGmC*SfA*SfU*SfU*SfU*SfC | | OOSSSSSS | PS2 after nucleotide 11; | |
| | | | *SfU | | | two SfG * on 5′ wing | |
| | | | | | | converted to fG-PS2 | |
| WV- | UCAAGGAAGA | 658 | fU*SfC*SfA*SfA*mG:mG:mAmAmGm | 1410 | SSSXDDOOOOD | WV-2438 based, with | Exon 51 |
| 3075 | UGGCAUUUCU | | AmU:mGmGmC*SfA*SfU*SfU*SfU*Sf | | OOSSSSSS | PS2 after nucleotide 11; | |
| | | | C*SfU | | | two SfG * on 5′ wing | |
| | | | | | | converted to mG-PS2 | |
| WV- | UCAAGGAAGA | 659 | fU*SfC*SfA*SfA*SfG*SfG*SfA*SmAm | 1411 | SSSSSSSOOODO | WV-2749 based, with | Exon 51 |
| 3076 | UGGCAUUUCU | | GmAmU:mGmG*SfC*SfA*SfU*SfU*Sf | | SSSSSSS | PS2 after nucleotide 11 | |
| | | | U*SfC*SfU | | | | |
| WV- | UCAAGGAAGA | 660 | fU*SfC*SfA*SfA*fG:fG:fA*SmAmGmA | 1412 | SSSXDDSOOOD | WV-2749 based, with | Exon 51 |
| 3077 | UGGCAUUUCU | | mU:mGmG*SfC*SfA*SfU*SfU*SfU*Sf | | OSSSSSSS | PS2 after nucleotide 11; | |
| | | | C*SfU | | | two SfG * on 5′ wing | |
| | | | | | | converted to fG-PS2 | |
| WV- | UCAAGGAAGA | 661 | fU*SfC*SfA*SfA*mG:mG:fA*SmAmG | 1413 | SSSXDDSOOOD | WV-2749 based, with | Exon 51 |
| 3078 | UGGCAUUUCU | | mAmU:mGmG*SfC*SfA*SfU*SfU*SfU | | OSSSSSSS | PS2 after nucleotide 11; | |
| | | | *SfC*SfU | | | two SfG * on 5′ wing | |
| | | | | | | converted to mG-PS2 | |
| WV- | UCAAGGAAGA | 662 | fU*SfC*SfA*SfA*SfG*SfG*SfA*SfA*S | 1414 | SSSSSSSSOODSS | WV-2530 based, with | Exon 51 |
| 3079 | UGGCAUUUCU | | mGmAmU:mG*SfG*SfC*SfA*SfU*SfU | | SSSSSS | PS2 after nucleotide 11 | |
| | | | *SfU*SfC*SfU | | | | |
| WV- | UCAAGGAAGA | 663 | fU*SfC*SfA*SfA*SfG*SfG*SfA*SfA*S | 1415 | SSSSSSSSDDDSS | WV-2530 based, with | Exon 51 |
| 3080 | UGGCAUUUCU | | mG:mA:mU:mG*SfG*SfC*SfA*SfU*Sf | | SSSSSS | PS2 after nucleotide 9, | |
| | | | U*SfU*SfC*SfU | | | 10, 11 | |
| WV- | UCAAGGAAGA | 664 | fU*SfC*SfA*SfA*SfG*SfG*SfA*SfA*S | 1416 | SSSSSSSSDODSS | WV-2530 based, with | Exon 51 |
| 3081 | UGGCAUUUCU | | mG:mAmU:mG*SfG*SfC*SfA*SfU*SfU | | SSSSSS | PS2 after nucleotide 9, | |
| | | | *SfU*SfC*SfU | | | 11 | |
| WV- | UCAAGGAAGA | 665 | fU*SfC*SfA*SfA*fG:fG:fA*SfA*SmGm | 1417 | SSSXDDSSOODS | WV-2530 based, with | Exon 51 |
| 3082 | UGGCAUUUCU | | AmU:mG*SfG*SfC*SfA*SfU*SfU*SfU* | | SSSSSSS | PS2 after nucleotide 11; | |
| | | | SfC*SfU | | | two SfG * on 5′ wing | |
| | | | | | | converted to fG-PS2 | |
| WV- | UCAAGGAAGA | 666 | fU*SfC*SfA*SfA*mG:mG:fA*SfA*SmG | 1418 | SSSXDDSSOODS | WV-2530 based, with | Exon 51 |
| 3083 | UGGCAUUUCU | | mAmU:mG*SfG*SfC*SfA*SfU*SfU*Sf | | SSSSSSS | PS2 after nucleotide 11; | |
| | | | U*SfC*SfU | | | two SfG * on 5′ wing | |
| | | | | | | converted to mG-PS2 | |
| WV- | UCAAGGAAGA | 667 | Mod015L001mU*mC*mA*mA*mG*mG | 1419 | OOXXXXXXXX | WV942 with C6 PO and | Exon 51 |
| 3084 | UGGCAUUUCU | | *mA*mA*mG*mA*mU*mG*mG*mC* | | XXXXXXXXXXX | Stearic | |
| | | | mA*mU*mU*mU*mC*mU | | | | |
| WV- | UCAAGGAAGA | 668 | Mod019L001mU*mC*mA*mA*mG*mG | 1420 | OOXXXXXXXX | WV942 with C6 PO and | Exon 51 |
| 3085 | UGGCAUUUCU | | *mA*mA*mG*mA*mU*mG*mG*mC* | | XXXXXXXXXXX | gamma-Linolenic | |
| | | | mA*mU*mU*mU*mC*mU | | | | |
| WV- | UCAAGGAAGA | 669 | Mod020L001mU*mC*mA*mA*mG*mG | 1421 | OOXXXXXXXX | WV942 with C6 PO and | Exon 51 |
| 3086 | UGGCAUUUCU | | *mA*mA*mG*mA*mU*mG*mG*mC* | | XXXXXXXXXXX | Turbinaric | |
| | | | mA*mU*mU*mU*mC*mU | | | | |
| WV- | UCAAGGAAGA | 670 | Mod015L001:mU*mC*mA*mA*mG*m | 1422 | ODXXXXXXXX | WV942 with C6 PS2 | Exon 51 |
| 3087 | UGGCAUUUCU | | G*mA*mA*mG*mA*mU*mG*mG*mC* | | XXXXXXXXXXX | and Stearic | |
| | | | mA*mU*mU*mU*mC*mU | | | | |
| WV- | UCAAGGAAGA | 671 | Mod019L001:mU*mC*mA*mA*mG*m | 1423 | ODXXXXXXXX | WV942 with C6 PS2 | Exon 51 |
| 3088 | UGGCAUUUCU | | G*mA*mA*mG*mA*mU*mG*mG*mC* | | XXXXXXXXXXX | and gamma-Linolenic | |
| | | | mA*mU*mU*mU*mC*mU | | | | |
| WV- | UCAAGGAAGA | 672 | Mod020L001:mU*mC*mA*mA*mG*m | 1424 | ODXXXXXXXX | WV942 with C6 PS2 | Exon 51 |
| 3089 | UGGCAUUUCU | | G*mA*mA*mG*mA*mU*mG*mG*mC* | | XXXXXXXXXXX | and Turbinaric | |
| | | | mA*mU*mU*mU*mC*mU | | | | |
| WV- | UCAAGGAAGA | 673 | fU*SfC*SfA*SfA*SfG:fG:mAmAmGmA | 1425 | SSSSDDOOOOD | Variant of WV-3074. | Exon 51 |
| 3113 | UGGCAUUUCU | | mU:mGmGmC*SfA*SfU*SfU*SfU*SfC | | OOSSSSSS | There was a randomer | |
| | | | *SfU | | | PS in WV-3074 | |
| WV- | UCAAGGAAGA | 674 | fU*SfC*SfA*SfA*SmG:mG:mAmAmG | 1426 | SSSSDDOOOOD | Variant of WV-3075. | Exon 51 |
| 3114 | UGGCAUUUCU | | mAmU:mGmGmC*SfA*SfU*SfU*SfU* | | OOSSSSSS | There was a randomer | |
| | | | SfC*SfU | | | PS in WV-3075 | |
| WV- | UCAAGGAAGA | 675 | fU*SfC*SfA*SfA*SfG:fG:fA*SmAmGm | 1427 | SSSSDDSOOOD | Variant of WV-3077. | Exon 51 |
| 3115 | UGGCAUUUCU | | AmU:mGmG*SfC*SfA*SfU*SfU*SfU*S | | OSSSSSSS | There was a randomer | |
| | | | fC*SfU | | | PS in WV-3077 | |
| WV- | UCAAGGAAGA | 676 | fU*SfC*SfA*SfA*SmG:mG:fA*SmAmG | 1428 | SSSSDDSOOOD | Variant of WV-3078. | Exon 51 |
| 3116 | UGGCAUUUCU | | mAmU:mGmG*SfC*SfA*SfU*SfU*SfU | | OSSSSSSS | There was a randomer | |
| | | | *SfC*SfU | | | PS in WV-3078 | |
| WV- | UCAAGGAAGA | 677 | fU*SfC*SfA*SfA*SfG:fG:fA*SfA*SmG | 1429 | SSSSDDSSOODS | Variant of WV-3082. | Exon 51 |
| 3117 | UGGCAUUUCU | | mAmU:mG*SfG*SfC*SfA*SfU*SfU*Sf | | SSSSSSS | There was a randomer | |
| | | | U*SfC*SfU | | | PS in WV-3082 | |
| WV- | UCAAGGAAGA | 678 | fU*SfC*SfA*SfA*SmG:mG:fA*SfA*Sm | 1430 | SSSSDDSSOODS | Variant of WV-3083. | Exon 51 |
| 3118 | UGGCAUUUCU | | GmAmU:mG*SfG*SfC*SfA*SfU*SfU*S | | SSSSSSS | There was a randomer | |
| | | | fU*SfC*SfU | | | PS in WV-3083 | |
| WV- | UCAAGGAAGA | 679 | fU*SfC*SfA*SfA*SfG*SfG*SfA*SfA*Sf | 1431 | SSSSSSSSSOOSS | 9F-3OMe-8F 9Sp-2PO- | Exon 51 |
| 3120 | UGGCAUUUCU | | G*SmAmUmG*SfG*SfC*SfA*SfU*SfU | | SSSSSS | 8Sp | |
| | | | *SfU*SfC*SfU | | | | |
| WV- | UCAAGGAAGA | 680 | fU*fC*fA*fA*fG*fG*fA*fA*fG*mAmU | 1432 | XXXXXXXXXO | 9F-3OMe-8F 9PS-2PO- | Exon 51 |
| 3121 | UGGCAUUUCU | | mG*fG*fC*fA*fU*fU*fU*fC*fU | | OXXXXXXXX | 8PS, randomer version | |
| | | | | | | of WV-3120 | |
| WV- | UCAAGGAAGA | 681 | fU*SfC*SfA*SfA*SfG*SfG*SmAfA*Sm | 1433 | SSSSSSOSOSOS | WV-2438 modifed | DMD |
| 3152 | UGGCAUUUCU | | GfA*SmUfG*SmGfC*SfA*SfU*SfU*Sf | | OSSSSSS | | |
| | | | U*SfC*SfU | | | | |
| WV- | UCAAGGAAGA | 682 | fU*SfC*SfA*SfA*SfG*SfG*SfA*SfA*S | 1434 | SSSSSSSSOSOSS | WV-2529 modified | DMD |
| 3153 | UGGCAUUUCU | | mGfA*SmUfG*SmG*SfC*SfA*SfU*SfU | | SSSSSS | | |
| | | | *SfU*SfC*SfU | | | | |
| WV- | UCAAGGAAGA | 683 | L001mU*mC*mA*mA*mG*mG*mA*m | 1435 | OXXXXXXXXX | WV942 with C6 PO | Exon 51 |
| 3357 | UGGCAUUUCU | | A*mG*mA*mU*mG*mG*mC*mA*mU* | | XXXXXXXXXX | linker | |
| | | | mU*mU*mC*mU | | | | |
| WV- | UCAAGGAAGA | 684 | L001fU*SfC*SfA*SfA*SfG*SfG*SfA*Sf | 1436 | OSSSSSSSSSOSS | WV2531 with C6 PO | Exon 51 |
| 3358 | UGGCAUUUCU | | A*SfG*SmAmU*SfG*SfG*SfC*SfA*Sf | | SSSSSSS | linker | |
| | | | U*SfU*SfU*SfC*SfU | | | | |
| WV- | UCAAGGAAGA | 685 | Mod013L001mU*mC*mA *mA*mG*mG | 1437 | OOXXXXXXXX | WV942 with C6 amine | Exon 51 |
| 3359 | UGGCAUUUCU | | *mA*mA*mG*mA*mU*mG*mG*mC* | | XXXXXXXXXXX | PO linker, Lauric acid | |
| | | | mA*mU*mU*mU*mC*mU | | | | |
| WV- | UCAAGGAAGA | 686 | Mod013L001fU*SfC*SfA*SfA*SfG*SfG | 1438 | OOSSSSSSSSSOS | WV2531 with C6 amine | Exon 51 |
| 3360 | UGGCAUUUCU | | *SfA*SfA*SfG*SmAmU*SfG*SfG*SfC* | | SSSSSSSS | PO linker, Lauric acid | |
| | | | SfA*SfU*SfU*SfU*SfC*SfU | | | | |
| WV- | UCAAGGAAGA | 687 | Mod014L001fU*SfC*SfA*SfA*SfG*SfG | 1439 | OOSSSSSSSSSOS | WV2531 with C6 amine | Exon 51 |
| 3361 | UGGCAUUUCU | | *SfA*SfA*SfG*SmAmU*SfG*SfG*SfC* | | SSSSSSSS | PO linker, Myristic acid | |
| | | | SfA*SfU*SfU*SfU*SfC*SfU | | | | |
| WV- | UCAAGGAAGA | 688 | Mod005L001fU*SfC*SfA*SfA*SfG*SfG | 1440 | OOSSSSSSSSSOS | WV2531 with C6 amine | Exon 51 |
| 3362 | UGGCAUUUCU | | *SfA*SfA*SfG*SmAmU*SfG*SfG*SfC* | | SSSSSSSS | PO linker, Palmitic acid | |
| | | | SfA*SfU*SfU*SfU*SfC*SfU | | | | |
| WV- | UCAAGGAAGA | 689 | Mod015L001fU*SfC*SfA*SfA*SfG*SfG | 1441 | OOSSSSSSSSSOS | WV2531 with C6 amine | Exon 51 |
| 3363 | UGGCAUUUCU | | *SfA*SfA*SfG*SmAmU*SfG*SfG*SfC* | | SSSSSSSS | PO linker, Stearic acid | |
| | | | SfA*SfU*SfU*SfU*SfC*SfU | | | | |
| WV- | UCAAGGAAGA | 690 | Mod020L001fU*SfC*SfA*SfA*SfG*SfG | 1442 | OOSSSSSSSSSOS | WV2531 with C6 amine | Exon 51 |
| 3364 | UGGCAUUUCU | | *SfA*SfA*SfG*SmAmU*SfG*SfG*SfC* | | SSSSSSSS | PO linker, Turbinaric | |
| | | | SfA*SfU*SfU*SfU*SfC*SfU | | | acid | |
| WV- | UCAAGGAAGA | 691 | Mod027L001fU*SfC*SfA*SfA*SfG*SfG | 1443 | OSSSSSSSSSOSS | WV2531 with C6 amine | Exon 51 |
| 3365 | UGGCAUUUCU | | *SfA*SfA*SfG*SmAmU*SfG*SfG*SfC* | | SSSSSSS | PO linker, | |
| | | | SfA*SfU*SfU*SfU*SfC*SfU | | | MonoSulfonamide | |
| WV- | UCAAGGAAGA | 692 | Mod029L001fU*SfC*SfA*SfA*SfG*SfG | 1444 | OSSSSSSSSSOSS | WV2531 with C6 amine | Exon 51 |
| 3366 | UGGCAUUUCU | | *SfA*SfA*SfG*SmAmU*SfG*SfG*SfC* | | SSSSSSS | PO linker, | |
| | | | SfA*SfU*SfU*SfU*SfC*SfU | | | TriSulfonamide | |
| WV- | UCAAGGAAGA | 693 | fU*SfC*SfA*SfA*SfG*SfGfA*SmAfG* | 1445 | SSSSSOSOSOSO | modifying WV-3152, | Exon 51 |
| 3463 | UGGCAUUUCU | | SmAfU*SmGfGfC*SfA*SfU*SfU*SfU* | | OSSSSSS | 2′f-U and Sp in the | |
| | | | SfC*SfU | | | middle | |
| WV- | UCAAGGAAGA | 694 | fU*SfC*SfA*SfA*SfG*SfG*SfA*SfAfG | 1446 | SSSSSSSOSOSSS | modifying WV-3153, | Exon 51 |
| 3464 | UGGCAUUUCU | | *SmAfU*SmG*SmG*SfC*SfA*SfU*SfU | | SSSSSS | 2′f-U and Sp in the | |
| | | | *SfU*SfC*SfU | | | middle | |
| WV- | UCAAGGAAGA | 695 | fU*SfC*SfA*SfA*SfG*SfG*SfA*SfA*Sf | 1447 | SSSSSSSSSOSSS | modifying WV-2531, | Exon 51 |
| 3465 | UGGCAUUUCU | | G*SmAfU*SmG*SfG*SfC*SfA*SfU*Sf | | SSSSSS | 2′f-U and Sp in the | |
| | | | U*SfU*SfC*SfU | | | middle | |
| WV- | UCAAGGAAGA | 696 | fU*SfC*SfA*SfA*SfG*SfG*SfA*SfA*S | 1448 | SSSSSSSSOOSOS | modifying WV-3028, | Exon 51 |
| 3466 | UGGCAUUUCU | | mGmAfU*SmGmG*SfC*SfA*SfU*SfU* | | SSSSSS | 2′f-U and Sp in the | |
| | | | SfU*SfC*SfU | | | middle | |
| WV- | UCAAGGAAGA | 697 | fU*SfC*SfA*SfA*SfG*SfG*SfA*SfA*Sf | 1449 | SSSSSSSSSOSOS | modifying WV-3120, | Exon 51 |
| 3467 | UGGCAUUUCU | | G*SmAfU*SmGfG*SfC*SfA*SfU*SfU* | | SSSSSS | 2′f-U and Sp in the | |
| | | | SfU*SfC*SfU | | | middle | |
| WV- | UCAAGGAAGA | 698 | fU*SfC*SfA*SfA*SfG*SfG*mAmAmG | 1450 | SSSSSXOOOOSO | modifying WV-3046, | Exon 51 |
| 3468 | UGGCAUUUCU | | mAfU*SmGmG*SfC*SfA*SfU*SfU*SfU | | SSSSSSS | 2′f-U and Sp in the | |
| | | | *SfC*SfU | | | middle | |
| WV- | UCAAGGAAGA | 699 | fU*SfC*SfA*SfA*SfG*SfG*SmA*SmA* | 1451 | SSSSSSSSSSSSSS | modifying WV-3047, | Exon 51 |
| 3469 | UGGCAUUUCU | | SmG*SmA*SfU*SmG*SmG*SfC*SfA*S | | SSSSS | 2′f-U and Sp in the | |
| | | | fU*SfU*SfU*SfC*SfU | | | middle | |
| WV- | UCAAGGAAGA | 700 | fU*SfC*SfA*SfA*SfG*SfG*SmAfA*Sm | 1452 | SSSSSSOSOSOS | 2′F on the middle U; | DMD |
| 3470 | UGGCAUUUCU | | GfA*SfUfG*SmGfC*SfA*SfU*SfU*SfU | | OSSSSSS | modified on WV-3152 | |
| | | | *SfC*SfU | | | | |
| WV- | UCAAGGAAGA | 701 | fU*SfC*SfA*SfA*SfG*SfG*SmAfA*Sm | 1453 | SSSSSSOSOSOO | 2′F on the middle U; | DMD |
| 3471 | UGGCAUUUCU | | GfA*SfUmGmGfC*SfA*SfU*SfU*SfU* | | OSSSSSS | modified on WV-3152 | |
| | | | SfC*SfU | | | | |
| WV- | UCAAGGAAGA | 702 | fU*SfC*SfA*SfA*SfG*SfG*SmAfA*Sm | 1454 | SSSSSSOSOSSO | 2′F on the middle U; | DMD |
| 3472 | UGGCAUUUCU | | GfA*SfU*SmGmGfC*SfA*SfU*SfU*Sf | | OSSSSSS | modified on WV-3152 | |
| | | | U*SfC*SfU | | | | |
| WV- | UCAAGGAAGA | 703 | fU*SfC*SfA*SfA*SfG*SfG*SmAfA*Sm | 1455 | SSSSSSOSOSSO | 2′F on the middle U; | DMD |
| 3473 | UGGCAUUUCU | | GmA*SfU*SmGmGfC*SfA*SfU*SfU*Sf | | OSSSSSS | modified on WV-3152 | |
| | | | U*SfC*SfU | | | | |
| WV- | UCAAGGAAGA | 704 | fU*SfC*SfA*SfA*SfG*SfG*SmAfA*Sm | 1456 | SSSSSSOSOOSO | modifed on WV-3472; | Exon 51 |
| 3506 | UGGCAUUUCU | | GfAfU*SmGmGfC*SfA*SfU*SfU*SfU* | | OSSSSSS | except for PO linker | |
| | | | SfC*SfU | | | between fA (10th nt) and | |
| | | | | | | fU (11th nt) | |
| WV- | UCAAGGAAGA | 705 | fU*SfC*SfA*SfA*SfG*SfG*SmAfA*Sm | 1457 | SSSSSSOSOOSO | modifed on WV-3473; | Exon 51 |
| 3507 | UGGCAUUUCU | | GmAfU*SmGmGfC*SfA*SfU*SfU*SfU | | OSSSSSS | except for PO linker | |
| | | | *SfC*SfU | | | between mA (10th nt) | |
| | | | | | | and fU (11th nt) | |
| WV- | UCAAGGAAGA | 706 | fU*SfC*SfA*SfA*SfG*SfG*SmAfA*Sm | 1458 | SSSSSSOSOSSO | modifed on WV-3472; | Exon 51 |
| 3508 | UGGCAUUUCU | | GfA*SfU*SmGmGfC*SfAfU*SfU*SfU* | | OSOSSSS | except for PO linker | |
| | | | SfC*SfU | | | between fA (15th nt) and | |
| | | | | | | fU (16th nt) | |
| WV- | UCAAGGAAGA | 707 | fU*SfC*SfA*SfA*SfG*SfG*SmAfA*Sm | 1459 | SSSSSSOSOSSO | modifed on WV-3473; | Exon 51 |
| 3509 | UGGCAUUUCU | | GmA*SfU*SmGmGfC*SfAfU*SfU*SfU | | OSOSSSS | except for PO linker | |
| | | | *SfC*SfU | | | between fA (15th nt) and | |
| | | | | | | fU (16th nt) | |
| WV- | UCAAGGAAGA | 708 | fU*SfC*SfA*SfA*SfG*SfG*SmAfA*Sm | 1460 | SSSSSSOSOOSO | modifed on WV-3472; | Exon 51 |
| 3510 | UGGCAUUUCU | | GfAfU*SmGmGfC*SmA*SfU*SfU*SfU | | OSSSSSS | except for mA on 15th nt | |
| | | | *SfC*SfU | | | | |
| WV- | UCAAGGAAGA | 709 | fU*SfC*SfA*SfA*SfG*SfG*SmAfA*Sm | 1461 | SSSSSSOSOOSO | modifed on WV-3473; | Exon 51 |
| 3511 | UGGCAUUUCU | | GmAfU*SmGmGfC*SmA*SfU*SfU*Sf | | OSSSSSS | except for mA on 15th nt | |
| | | | U*SfC*SfU | | | | |
| WV- | UCAAGGAAGA | 710 | fU*SfC*SfA*SfA*SfG*SfG*SmAfA*Sm | 1462 | SSSSSSOSOOSO | modifed on WV-3472; | Exon 51 |
| 3512 | UGGCAUUUCU | | GfAfU*SmGmGfC*SmAfU*SfU*SfU*Sf | | OSOSSSS | except for PO linker | |
| | | | C*SfU | | | between fA (10th nt) and | |
| | | | | | | fU (11th nt); mA on 15th | |
| | | | | | | nt, and PO between mA | |
| | | | | | | (15th nt) and fU (16th | |
| | | | | | | nt) | |
| WV- | UCAAGGAAGA | 711 | fU*SfC*SfA*SfA*SfG*SfG*SmAfA*Sm | 1463 | SSSSSSOSOOSO | modifed on WV-3472; | Exon 51 |
| 3513 | UGGCAUUUCU | | GmAfU*SmGmGfC*SmAfU*SfU*SfU* | | OSOSSSS | except for PO linker | |
| | | | SfC*SfU | | | between mA (10th nt) | |
| | | | | | | and fU (11th nt); mA on | |
| | | | | | | 15th nt, and PO between | |
| | | | | | | mA (15th nt) and fU | |
| | | | | | | (16th nt) | |
| WV- | UCAAGGAAGA | 712 | fU*SfC*SfA*SfA*SfG*SfG*SmAfA*Sm | 1464 | SSSSSSOSOOSO | modifed on WV-3472; | Exon 51 |
| 3514 | UGGCAUUUCU | | GfAfU*SmGmGfC*SfAfU*SfU*SfU*Sf | | OSOSSSS | except for PO linker | |
| | | | C*SfU | | | between fA (10th nt) and | |
| | | | | | | fU (11th nt); PO | |
| | | | | | | between fA (15th nt) and | |
| | | | | | | fU (16th nt) | |
| WV- | UCAAGGAAGA | 713 | fU*SfC*SfA*SfA*SfG*SfG*SmAfA*Sm | 1465 | SSSSSSOSOOSO | modifed on WV-3472; | Exon 51 |
| 3515 | UGGCAUUUCU | | GmAfU*SmGmGfC*SfAfU*SfU*SfU*Sf | | OSOSSSS | except for PO linker | |
| | | | C*SfU | | | between mA (10th nt) | |
| | | | | | | and fU (11th nt); PO | |
| | | | | | | between fA (15th nt) and | |
| | | | | | | fU (16th nt) | |
| WV- | UCAAGGAAGA | 714 | fU*fC*fA*fA*fG*fG*mAfA*mGfA*mU | 1466 | XXXXXXOXOX | randomer version of | Exon 51 |
| 3516 | UGGCAUUUCU | | fG*mGfC*fA*fU*fU*fU*fC*fU | | OXOXXXXXX | WV-3152 | |
| WV- | UCAAGGAAGA | 715 | Mod030fU*fC*fA*fA*fG*fG*mAfA*m | 1467 | OXXXXXXOXO | with PO linker, Lauric | Exon 51 |
| 3517 | UGGCAUUUCU | | GfA*mUfG*mGfC*fA*fU*fU*fU*fC*fU | | XOXOXXXXXX | | |
| WV- | UCAAGGAAGA | 716 | Mod031fU*fC*fA*fA*fG*fG*mAfA*m | 1468 | OXXXXXXOXO | with PO linker, Myristic | Exon 51 |
| 3518 | UGGCAUUUCU | | GfA*mUfG*mGfC*fA*fU*fU*fU*fC*fU | | XOXOXXXXXX | | |
| WV- | UCAAGGAAGA | 717 | Mod032fU*fC*fA*fA*fG*fG*mAfA*m | 1469 | OXXXXXXOXO | with PO linker, Palmitic | Exon 51 |
| 3519 | UGGCAUUUCU | | GfA*mUfG*mGfC*fA*fU*fU*fU*fC*fU | | XOXOXXXXXX | | |
| WV- | UCAAGGAAGA | 718 | Mod033fU*fC*fA*fA*fG*fG*mAfA*m | 1470 | OXXXXXXOXO | with PO linker, Stearic | Exon 51 |
| 3520 | UGGCAUUUCU | | GfA*mUfG*mGfC*fA*fU*fU*fU*fC*fU | | XOXOXXXXXX | | |
| WV- | UCAAGGAAGA | 719 | Mod013L001fU*SfC*SfA*SfA*SfG*SfG | 1471 | OOSSSSSSOSOS | WV-3473, Lauric acid, | Exon 51 |
| 3543 | UGGCAUUUCU | | *SmAfA*SmGmA*SfU*SmGmGfC*SfA | | SOOSSSSSS | C6 PO linker | |
| | | | *SfU*SfU*SfU*SfC*SfU | | | | |
| WV- | UCAAGGAAGA | 720 | Mod005L001fU*SfC*SfA*SfA*SfG*SfG | 1472 | OOSSSSSSOSOS | WV-3473, Palmitic acid, | Exon 51 |
| 3544 | UGGCAUUUCU | | *SmAfA*SmGmA*SfU*SmGmGfC*SfA | | SOOSSSSSS | C6 PO linker | |
| | | | *SfU*SfU*SfU*SfC*SfU | | | | |
| WV- | UCAAGGAAGA | 721 | Mod015L001fU*SfC*SfA*SfA*SfG*SfG | 1473 | OOSSSSSSOSOS | WV-3473, Stearic acid, | Exon 51 |
| 3545 | UGGCAUUUCU | | *SmAfA*SmGmA*SfU*SmGmGfC*SfA | | SOOSSSSSS | C6 PO linker | |
| | | | *SfU*SfU*SfU*SfC*SfU | | | | |
| WV- | UCAAGGAAGA | 722 | Mod020L001fU*SfC*SfA*SfA*SfG*SfG | 1474 | OOSSSSSSOSOS | WV-3473, Turbinaric | Exon 51 |
| 3546 | UGGCAUUUCU | | *SmAfA*SmGmA*SfU*SmGmGfC*SfA | | SOOSSSSSS | acid, C6 PO linker | |
| | | | *SfU*SfU*SfU*SfC*SfU | | | | |
| WV- | UCAAGGAAGA | 723 | Mod027L001fU*SfC*SfA*SfA*SfG*SfG | 1475 | OSSSSSSOSOSS | WV-3473, | Exon 51 |
| 3547 | UGGCAUUUCU | | *SmAfA*SmGmA*SfU*SmGmGfC*SfA | | OOSSSSSS | Monosulfonamide, C6 | |
| | | | *SfU*SfU*SfU*SfC*SfU | | | PO linker | |
| WV- | UCAAGGAAGA | 724 | Mod029L001fU*SfC*SfA*SfA*SfG*SfG | 1476 | OSSSSSSOSOSS | WV-3473, | Exon 51 |
| 3548 | UGGCAUUUCU | | *SmAfA*SmGmA*SfU*SmGmGfC*SfA | | OOSSSSSS | Trisulfonamide, C6 PO | |
| | | | *SfU*SfU*SfU*SfC*SfU | | | linker | |
| WV- | UCAAGGAAGA | 725 | Mod030fU*SfC*SfA*SfA*SfG*SfG*Sm | 1477 | OSSSSSSOSOSS | WV-3473, Laurie, PO | Exon 51 |
| 3549 | UGGCAUUUCU | | AfA*SmGmA*SfU*SmGmGfC*SfA*Sf | | OOSSSSSS | linker | |
| | | | U*SfU*SfU*SfC*SfU | | | | |
| WV- | UCAAGGAAGA | 726 | Mod032fU*SfC*SfA*SfA*SfG*SfG*Sm | 1478 | OSSSSSSOSOSS | WV-3473, Palmitic, PO | Exon 51 |
| 3550 | UGGCAUUUCU | | AfA*SmGmA*SfU*SmGmGfC*SfA*Sf | | OOSSSSSS | linker | |
| | | | U*SfU*SfU*SfC*SfU | | | | |
| WV- | UCAAGGAAGA | 727 | Mod033fU*SfC*SfA*SfA*SfG*SfG*Sm | 1479 | OSSSSSSOSOSS | WV-3473, Stearic, PO | Exon 51 |
| 3551 | UGGCAUUUCU | | AfA*SmGmA*SfU*SmGmGfC*SfA*Sf | | OOSSSSSS | linker | |
| | | | U*SfU*SfU*SfC*SfU | | | | |
| WV- | UCAAGGAAGA | 728 | Mod020L001*fU*SfC*SfA*SfA*SfG*Sf | 1480 | OXSSSSSSOSOS | WV-3473, Turbinaric | Exon 51 |
| 3552 | UGGCAUUUCU | | G*SmAfA*SmGmA*SfU*SmGmGfC*Sf | | SOOSSSSSS | acid, C6 PS linker | |
| | | | A*SfU*SfU*SfU*SfC*SfU | | | | |
| WV- | UCAAGGAAGA | 729 | Mod005L001*fU*SfC*SfA*SfA*SfG*Sf | 1481 | OXSSSSSSOSOS | WV-3473, Palmitic acid, | Exon 51 |
| 3553 | UGGCAUUUCU | | G*SmAfA*SmGmA*SfU*SmGmGfC*Sf | | SOOSSSSSS | C6 PS linker | |
| | | | A*SfU*SfU*SfU*SfC*SfU | | | | |
| WV- | UCAAGGAAGA | 730 | Mod014L001fU*SfC*SfA*SfA*SfG*SfG | 1482 | OOSSSSSSOSOS | WV-3473, Myristic acid, | Exon 51 |
| 3554 | UGGCAUUUCU | | *SmAfA*SmGmA*SfU*SmGmGfC*SfA | | SOOSSSSSS | C6 PO linker | |
| | | | *SfU*SfU*SfU*SfC*SfU | | | | |
| WV- | UCAAGGAAGA | 731 | Mod030*fU*SfC*SfA*SfA*SfG*SfG*S | 1483 | XSSSSSSOSOSS | WV-3473, Laurie PS | Exon 51 |
| 3555 | UGGCAUUUCU | | mAfA*SmGmA*SfU*SmGmGfC*SfA*S | | OOSSSSSS | linker | |
| | | | fU*SfU*SfU*SfC*SfU | | | | |
| WV- | UCAAGGAAGA | 732 | Mod032*fU*SfC*SfA*SfA*SfG*SfG*S | 1484 | XSSSSSSOSOSS | WV-3473, Palmitic PS | Exon 51 |
| 3556 | UGGCAUUUCU | | mAfA*SmGmA*SfU*SmGmGfC*SfA*5 | | OOSSSSSS | linker | |
| | | | fU*SfU*SfU*SfC*SfU | | | | |
| WV- | UCAAGGAAGA | 733 | Mod033*fU*SfC*SfA*SfA*SfG*SfG*S | 1485 | XSSSSSSOSOSS | WV-3473, Stearic PS | Exon 51 |
| 3557 | UGGCAUUUCU | | mAfA*SmGmA*SfU*SmGmGfC*SfA*5 | | OOSSSSSS | linker | |
| | | | fU*SfU*SfU*SfC*SfU | | | | |
| WV- | UCAAGGAAGA | 734 | Mod033*fU*fC*fA*fA*fG*fG*mAfA*m | 1486 | XXXXXXXOXO | with PS linker, Stearic | Exon 51 |
| 3558 | UGGCAUUUCU | | GfA*mUfG*mGfC*fA*fU*fU*fU*fC*fU | | XOXOXXXXXX | linker | |
| WV- | UCAAGGAAGA | 735 | Mod020L001fU*fC*fA*fA*fG*fG*mAf | 1487 | OOXXXXXXOX | with C6 amine PO | Exon 51 |
| 3559 | UGGCAUUUCU | | A*mGfA*mUfG*mGfC*fA*fU*fU*fU*f | | OXOXOXXXXXX | linker, Turbinaric acid | |
| WV- | UCAAGGAAGA | 736 | Mod020L001*fU*fC*fA*fA*fG*fG*mAf | 1488 | OXXXXXXXOX | with C6 amine PS linker, | Exon 51 |
| 3560 | UGGCAUUUCU | | A*mGfA*mUfG*mGfC*fA*fU*fU*fU*f | | OXOXOXXXXXX | Turbinaric acid | |
| | | | C*fU | | | | |
| WV- | UCAAGGAAGA | 737 | L001*fU*SfC*SfA*SfA*SfG*SfG*SmAf | 1489 | XSSSSSSOSOSS | WV-3473, C6 PS linker | Exon 51 |
| 3753 | UGGCAUUUCU | | A* SmGmA*SfU*SmGmGfC*SfA*SfU* | | OOSSSSSS | | |
| | | | SfU*SfU*SfC*SfU | | | | |
| WV- | UCAAGGAAGA | 738 | L001fU*SfC*SfA*SfA*SfG*SfG*SmAf | 1490 | OSSSSSSOSOSS | WV-3473, C6 PO linker | Exon 51 |
| 3754 | UGGCAUUUCU | | A* SmGmA*SfU*SmGmGfC*SfA*SfU* | | OOSSSSSS | | |
| | | | SfU*SfU*SfC*SfU | | | | |
| WV- | GCCAACUGGG | 739 | rGrCrCrArArCrUrGrGrGrArGrCrUrGrGr | 1491 | OOOOOOOOOO | Complementary RNA | MSTN |
| 3812 | AGCUGGAGCG | | ArGrCrGrCrArCrCrArArCrCrArG | | OOOOOOOOOO | | |
| | CACCAACCAG | | | | OOOOOOOOO | | |
| WV- | UCAAGGAAGA | 740 | L001*fU*fC*fA*fA*fG*fG*mAfA*mGf | 1492 | XXXXXXXOXO | WV-3516, C6 PS linker | Exon 51 |
| 3820 | UGGCAUUUCU | | A*mUfG*mGfC*fA*fU*fU*fU*fC*fU | | XOXOXXXXXX | | |
| WV- | UCAAGGAAGA | 741 | L001fU*fC*fA*fA*fG*fG*mAfA*mGfA | 1493 | OXXXXXXOXO | WV-3516, C6 PO linker | Exon 51 |
| 3821 | UGGCAUUUCU | | *mUfG*mGfC*fA*fU*fU*fU*fC*fU | | XOXOXXXXXX | | |
| WV- | UCAAGGAAGA | 742 | Mod015L001*fU*fC*fA*fA*fG*fG*mAf | 1494 | OXXXXXXXOX | WV-3516, C6 PS linker, | Exon 51 |
| 3855 | UGGCAUUUCU | | A*mGfA*mUfG*mGfC*fA*fU*fU*fU*f | | OXOXOXXXXXX | Stearic acid | |
| | | | C*fU | | | | |
| WV- | UCAAGGAAGA | 743 | Mod015L001fU*fC*fA*fA*fG*fG*mAf | 1495 | OOXXXXXXOX | WV-3516, C6 PO linker, | Exon 51 |
| 3856 | UGGCAUUUCU | | A*mGfA*mUfG*mGfC*fA*fU*fU*fU*f | | OXOXOXXXXXX | Stearic acid | |
| | | | C*fU | | | | |
| WV- | CCUUCCCUGAA | 744 | fC*fC*fU*fU*fC*fC*mCfU*GmAfA*m | 1496 | XXXXXXOXOO | Negative control | NA |
| 3975 | GGUUCCUCC | | GmGfU*fU*fC*fC*fU*fC*fC | | XOOXXXXXX | | |
| WV- | CCUUCCCUGAA | 745 | L001fC*fC*fU*fU*fC*fC*mCfU*GmAf | 1497 | OXXXXXXOXO | Negative control | NA |
| 3976 | GGUUCCUCC | | A*mGmGfU*fU*fC*fC*fU*fC*fC | | OXOOXXXXXX | | |
| WV- | CCUUCCCUGAA | 746 | Mod020L001fC*fC*fU*fU*fC*fC*mCfU | 1498 | OOXXXXXXOX | Negative control | NA |
| 3977 | GGUUCCUCC | | *GmAfA*mGmGfU*fU*fC*fC*fU*fC*fC | | OOXOOXXXXXX | | |
| WV- | CCUUCCCUGAA | 747 | fC*fC*fU*fU*fC*fC*mCfU*mGmAfA* | 1499 | XXXXXXOXOO | Negative control | NA |
| 3978 | GGUUCCUCC | | mGmGfU*fU*fC*fC*fU*fC*fC | | XOOXXXXXX | | |
| WV- | CCUUCCCUGAA | 748 | L001fC*fC*fU*fU*fC*fC*mCfU*mGm | 1500 | OXXXXXXOXO | Negative control | NA |
| 3979 | GGUUCCUCC | | AfA*mGmGfU*fU*fC*fC*fU*fC*fC | | OXOOXXXXXX | | |
| WV- | CCUUCCCUGAA | 749 | Mod020L001fC*fC*fU*fU*fC*fC*mCfU | 1501 | OOXXXXXXOX | Negative control | NA |
| 3980 | GGUUCCUCC | | *mGmAfA*mGmGfU*fU*fC*fC*fU*fC | | OOXOOXXXXXX | | |
| | | | *fC | | | | |
| WV- | UCAAGGAAGA | 750 | Mod015L001*fU*SfC*SfA*SfA*SfG*Sf | 1502 | OXSSSSSSOSOS | WV-3473, C6 PS linker, | Exon 51 |
| 4106 | UGGCAUUUCU | | G*SmAfA*SmGmA*SfU*SmGmGfC*Sf | | SOOSSSSSS | Stearic acid | |
| | | | A*SfU*SfU*SfU*SfC*SfU | | | | |
| WV- | UCAAGGAAGA | 751 | Mod015L001*SfU*SfC*SfA*SfA*SfG*S | 1503 | OSSSSSSSOSOSS | WV-3473, Sp stereopure | DMD |
| 4107 | UGGCAUUUCU | | fG*SmAfA*SmGmA*SfU*SmGmGfC*S | | OOSSSSSS | C6 linker, stearic acid | |
| | | | fA*SfU*SfU*SfU*SfC*SfU | | | | |
| WV- | UCAAGGAAGA | 752 | L001 * SfU * SfC * SfA * SfA * SfG * | 1504 | SSSSSSSOSOSSO | WV-3473, C6 and Sp | DMD |
| 4191 | UGGCAUUUCU | | SfG * SmAfA * SmGmA * SfU * | | OSSSSSS | stereopure linker | |
| | | | SmGmGfC * SfA * SfU * SfU * SfU * | | | | |
| | | | SfC * SfU | | | | |
| WV- | UCAAGGAAGA | 753 | fU*SfC*SfA*SfA*SfG*SfG*SmAfA*Sm | 1505 | SSSSSSOSOSSO | WV-3473 based, n-1 on | DMD |
| 4231 | UGGCAUUUC | | GmA*SfU*SmGmGfC*SfA*SfU*SfU*Sf | | OSSSSS | 3′ | |
| | | | U*SfC | | | | |
| WV- | UCAAGGAAGA | 754 | fU*SfC*SfA*SfA*SfG*SfG*SmAfA*Sm | 1506 | SSSSSSOSOSSO | WV-3473 based, n-2 on | DMD |
| 4232 | UGGCAUUU | | GmA*SfU*SmGmGfC*SfA*SfU*SfU*SfU | | OSSSS | 3′ | |
| WV- | CAAGGAAGAU | 755 | fC*SfA*SfA*SfG*SfG*SmAfA*SmGmA | 1507 | SSSSSOSOSSOO | WV-3473 based, n-1 on | DMD |
| 4233 | GGCAUUUCU | | *SfU*SmGmGfC*SfA*SfU*SfU*SfU*Sf | | SSSSSS | 5′ | |
| | | | C*SfU | | | | |
| WV- | GGCCAAACCUC | 756 | Mod020L001mG*mG*mC*mC*mA*mA | 1508 | OOXXXXXXXX | WV-943, C6 linker and | DMD |
| 4610 | GGCUUACCU | | *mA*mC*mC*mU*mC*mG*mG*mC*m | | XXXXXXXXXXX | PO, Turbinaric acid | mouse |
| | | | U*mU*mA*mC*mC*mU | | | | Exon23 |
| WV- | GGCCAAACCUC | 757 | Mod015L001mG*mG*mC*mC*mA*mA | 1509 | OOXXXXXXXX | WV-943, C6 linker and | DMD |
| 4611 | GGCUUACCU | | *mA*mC*mC*mU*mC*mG*mG*mC*m | | XXXXXXXXXXX | PO, Stearic acid | mouse |
| | | | U*mU*mA*mC*mC*mU | | | | Exon23 |
| WV- | UUCUGUAAGG | 758 | fU*fU*fC*fU*fG*fU*mA*mA*mG*mG | 1510 | XXXXXXXXXX | DMD mouse Exon23 | DMD |
| 4614 | UUUUUAUGUG | | *mU*mU*mU*mU*fU*fA*fU*fG*fU*fG | | XXXXXXXXX | | |
| WV- | AUUUCUGUAA | 759 | fA*fU*fU*fU*fC*fU*mG*mU*mA*mA | 1511 | XXXXXXXXXX | DMD mouse Exon23 | DMD |
| 4615 | GGUUUUUAUG | | *mG*mG*mU*mU*fU*fU*fU*fA*fU*fG | | XXXXXXXXX | | |
| WV- | CCAUUUCUGU | 760 | fC*fC*fA*fU*fU*fU*mC*mU*mG*mU* | 1512 | XXXXXXXXXX | DMD mouse Exon23 | DMD |
| 4616 | AAGGUUUUUA | | mA*mA*mG*mG*fU*fU*fU*fU*fU*fA | | XXXXXXXXX | | |
| WV- | AUCCAUUUCU | 761 | fA*fU*fC*fC*fA*fU*mU*mU*mC*mU* | 1513 | XXXXXXXXXX | DMD mouse Exon23 | DMD |
| 4617 | GUAAGGUUUU | | mG*mU*mA*mA*fG*fG*fU*fU*fU*fU | | XXXXXXXXX | | |
| WV- | CAUCCAUUUCU | 762 | fC*fA*fU*fC*fC*fA*mU*mU*mU*mC* | 1514 | XXXXXXXXXX | DMD mouse Exon23 | DMD |
| 4618 | GUAAGGUUU | | mU*mG*mU*mA*fA*fG*fG*fU*fU*fU | | XXXXXXXXX | | |
| WV- | CCAUCCAUUUC | 763 | fC*fC*fA*fU*fC*fC*mA*mU*mU*mU* | 1515 | XXXXXXXXXX | DMD mouse Exon23 | DMD |
| 4619 | UGUAAGGUU | | mC*mU*mG*mU*fA*fA*fG*fG*fU*fU | | XXXXXXXXX | | |
| WV- | GCCAUCCAUUU | 764 | fG*fC*fC*fA*fU*fC*mC*mA*mU*mU* | 1516 | XXXXXXXXXX | DMD mouse Exon23 | DMD |
| 4620 | CUGUAAGGU | | mU*mC*mU*mG*fU*fA*fA*fG*fG*fU | | XXXXXXXXX | | |
| WV- | AGCCAUCCAUU | 765 | fA*fG*fC*fC*fA*fU*mC*mC*mA*mU* | 1517 | XXXXXXXXXX | DMD mouse Exon23 | DMD |
| 4621 | UCUGUAAGG | | mU*mU*mC*mU*fG*fU*fA*fA*fG*fG | | XXXXXXXXX | | |
| WV- | CAGCCAUCCAU | 766 | fC*fA*fG*fC*fC*fA*mU*mC*mC*mA* | 1518 | XXXXXXXXXX | DMD mouse Exon23 | DMD |
| 4622 | UUCUGUAAG | | mU*mU*mU*mC*fU*fG*fU*fA*fA*fG | | XXXXXXXXX | | |
| WV- | UCAGCCAUCCA | 767 | fU*fC*fA*fG*fC*fC*mA*mU*mC*mC* | 1519 | XXXXXXXXXX | DMD mouse Exon23 | DMD |
| 4623 | UUUCUGUAA | | mA*mU*mU*mU*fC*fU*fG*fU*fA*fA | | XXXXXXXXX | | |
| WV- | UUCAGCCAUCC | 768 | fU*fU*fC*fA*fG*fC*mC*mA*mU*mC* | 1520 | XXXXXXXXXX | DMD mouse Exon23 | DMD |
| 4624 | AUUUCUGUA | | mC*mA*mU*mU*fU*fC*fU*fG*fU*fA | | XXXXXXXXX | | |
| WV- | CUUCAGCCAUC | 769 | fC*fU*fU*fC*fA*fG*mC*mC*mA*mU* | 1521 | XXXXXXXXXX | DMD mouse Exon23 | DMD |
| 4625 | CAUUUCUGU | | mC*mC*mA*mU*fU*fU*fC*ffl*fG*fU | | XXXXXXXXX | | |
| WV- | ACUUCAGCCAU | 770 | fA*fC*fU*fU*fC*fA*mG*mC*mC*mA* | 1522 | XXXXXXXXXX | DMD mouse Exon23 | DMD |
| 4626 | CCAUUUCUG | | mU*mC*mC*mA*fU*fU*fU*fC*fU*fG | | XXXXXXXXX | | |
| WV- | AACUUCAGCCA | 771 | fA*fA*fC*fU*fU*fC*mA*mG*mC*mC* | 1523 | XXXXXXXXXX | DMD mouse Exon23 | DMD |
| 4627 | UCCAUUUCU | | mA*mU*mC*mC*fA*fU*fU*fU*fC*fU | | XXXXXXXXX | | |
| WV- | CAACUUCAGCC | 772 | fC*fA*fA*fC*fU*fU*mC*mA*mG*mC* | 1524 | XXXXXXXXXX | DMD mouse Exon23 | DMD |
| 4628 | AUCCAUUUC | | mC*mA*mU*mC*fC*fA*fU*fU*fU*fC | | XXXXXXXXX | | |
| WV- | UCAACUUCAGC | 773 | fU*fC*fA*fA*fC*fU*mU*mC*mA*mG* | 1525 | XXXXXXXXXX | DMD mouse Exon23 | DMD |
| 4629 | CAUCCAUUU | | mC*mC*mA*mU*fC*fC*fA*fU*fU*fU | | XXXXXXXXX | | |
| WV- | AUCAACUUCA | 774 | fA*fU*fC*fA*fA*fC*mU*mU*mC*mA* | 1526 | XXXXXXXXXX | DMD mouse Exon23 | DMD |
| 4630 | GCCAUCCAUU | | mG*mC*mC*mA*fU*fC*fC*fA*fU*fU | | XXXXXXXXX | | |
| WV- | CAUCAACUUCA | 775 | fC*fA*fU*fC*fA*fA*mC*mU*mU*mC* | 1527 | XXXXXXXXXX | DMD mouse Exon23 | DMD |
| 4631 | GCCAUCCAU | | mA*mG*mC*mC*fA*fU*fC*fC*fA*fU | | XXXXXXXXX | | |
| WV- | ACAUCAACUUC | 776 | fA*fC*fA*fU*fC*fA*mA*mC*mU*mU* | 1528 | XXXXXXXXXX | DMD mouse Exon23 | DMD |
| 4632 | AGCCAUCCA | | mC*mA*mG*mC*fC*fA*fU*fC*fC*fA | | XXXXXXXXX | | |
| WV- | AACAUCAACU | 777 | fA*fA*fC*fA*fU*fC*mA*mA*mC*mU* | 1529 | XXXXXXXXXX | DMD mouse Exon23 | DMD |
| 4633 | UCAGCCAUCC | | mU*mC*mA*mG*fC*fC*fA*fU*fC*fC | | XXXXXXXXX | | |
| WV- | GAAAACAUCA | 778 | fG*fA*fA*fA*fA*fC*mA*mU*mC*mA | 1530 | XXXXXXXXXX | DMD mouse Exon23 | DMD |
| 4634 | ACUUCAGCCA | | *mA*mC*mU*mU*fC*fA*fG*fC*fC*fA | | XXXXXXXXX | | |
| WV- | CAGGAAAACA | 779 | fC*fA*fG*fG*fA*fA*mA*mA*mC*mA | 1531 | XXXXXXXXXX | DMD mouse Exon23 | DMD |
| 4635 | UCAACUUCAG | | *mU*mC*mA*mA*fC*fU*fU*fC*fA*fG | | XXXXXXXXX | | |
| WV- | UUUCAGGAAA | 780 | fU*fU*fU*fC*fA*fG*mG*mA*mA*mA | 1532 | XXXXXXXXXX | DMD mouse Exon23 | DMD |
| 4636 | ACAUCAACUU | | *mA*mC*mA*mU*fC*fA*fA*fC*fU*fU | | XXXXXXXXX | | |
| WV- | CUCUUUCAGG | 781 | fC*fU*fC*fU*fU*fU*mC*mA*mG*mG* | 1533 | XXXXXXXXXX | DMD mouse Exon23 | DMD |
| 4637 | AAAACAUCAA | | mA*mA*mA*mA*fC*fA*fU*fC*fA*fA | | XXXXXXXXX | | |
| WV- | UUCCUCUUUCA | 782 | fU*fU*fC*fC*fU*fC*mU*mU*mU*mC* | 1534 | XXXXXXXXXX | DMD mouse Exon23 | DMD |
| 4638 | GGAAAACAU | | mA*mG*mG*mA*fA*fA*fA*fC*fA*fU | | XXXXXXXXX | | |
| WV- | GCCAUUCCUCU | 783 | fG*fC*fC*fA*ftl*fU*mC*mC*mU*mC* | 1535 | XXXXXXXXXX | DMD mouse Exon23 | DMD |
| 4639 | UUCAGGAAA | | mU*mU*mU*mC*fA*fG*fG*fA*fA*fA | | XXXXXXXXX | | |
| WV- | GGCCAUUCCUC | 784 | fG*fG*fC*fC*fA*fU*mU*mC*mC*mU* | 1536 | XXXXXXXXXX | DMD mouse Exon23 | DMD |
| 4640 | UUUCAGGAA | | mC*mU*mU*mU*fC*fA*fG*fG*fA*fA | | XXXXXXXXX | | |
| WV- | AGGCCAUUCCU | 785 | fA*fG*fG*fC*fC*fA*mU*mU*mC*mC* | 1537 | XXXXXXXXXX | DMD mouse Exon23 | DMD |
| 4641 | CUUUCAGGA | | mU*mC*mU*mU*fU*fC*fA*fG*fG*fA | | XXXXXXXXX | | |
| WV- | CAGGCCAUUCC | 786 | fC*fA*fG*fG*fC*fC*mA*mU*mU*mC* | 1538 | XXXXXXXXXX | DMD mouse Exon23 | DMD |
| 4642 | UCUUUCAGG | | mC*mU*mC*mU*fU*fU*fC*fA*fG*fG | | XXXXXXXXX | | |
| WV- | GCAGGCCAUUC | 787 | fG*fC*fA*fG*fG*fC*mC*mA*mU*mU* | 1539 | XXXXXXXXXX | DMD mouse Exon23 | DMD |
| 4643 | CUCUUUCAG | | mC*mC*mU*mC*fU*fU*fU*fC*fA*fG | | XXXXXXXXX | | |
| WV- | GGCAGGCCAU | 788 | fG*fG*fC*fA*fG*fG*mC*mC*mA*mU* | 1540 | XXXXXXXXXX | DMD mouse Exon23 | DMD |
| 4644 | UCCUCUUUCA | | mU*mC*mC*mU*fC*fU*fU*fU*fC*fA | | XXXXXXXXX | | |
| WV- | GGGCAGGCCA | 789 | fG*fG*fG*fC*fA*fG*mG*mC*mC*mA* | 1541 | XXXXXXXXXX | DMD mouse Exon23 | DMD |
| 4645 | UUCCUCUUUC | | mU*mU*mC*mC*fU*fC*fU*fU*fU*fC | | XXXXXXXXX | | |
| WV- | AGGGCAGGCC | 790 | fA*fG*fG*fG*fC*fA*mG*mG*mC*mC* | 1542 | XXXXXXXXXX | DMD mouse Exon23 | DMD |
| 4646 | AUUCCUCUUU | | mA*mU*mU*mC*fC*fU*fC*fU*fU*fU | | XXXXXXXXX | | |
| WV- | CAGGGCAGGCC | 791 | fC*fA*fG*fG*fG*fC*mA*mG*mG*mC* | 1543 | XXXXXXXXXX | DMD mouse Exon23 | DMD |
| 4647 | AUUCCUCUU | | mC*mA*mU*mU*fC*fC*fU*fC*fU*fU | | XXXXXXXXX | | |
| WV- | CCAGGGCAGGC | 792 | fC*fC*fA*fG*fG*fG*mC*mA*mG*mG* | 1544 | XXXXXXXXXX | DMD mouse Exon23 | DMD |
| 4648 | CAUUCCUCU | | mC*mC*mA*mU*fU*fC*fC*fU*fC*fU | | XXXXXXXXX | | |
| WV- | CCCAGGGCAGG | 793 | fC*fC*fC*fA*fG*fG*mG*mC*mA*mG* | 1545 | XXXXXXXXXX | DMD mouse Exon23 | DMD |
| 4649 | CCAUUCCUC | | mG*mC*mC*mA*fU*fU*fC*fC*fU*fC | | XXXXXXXXX | | |
| WV- | CCCCAGGGCAG | 794 | fC*fC*fC*fC*fA*fG*mG*mG*mC*mA* | 1546 | XXXXXXXXXX | DMD mouse Exon23 | DMD |
| 4650 | GCCAUUCCU | | mG*mG*mC*mC*fA*fU*fU*fC*fC*fU | | XXXXXXXXX | | |
| WV- | CCCCCAGGGCA | 795 | fC*fC*fC*fC*fC*fA*mG*mG*mG*mC* | 1547 | XXXXXXXXXX | DMD mouse Exon23 | DMD |
| 4651 | GGCCAUUCC | | mA*mG*mG*mC*fC*fA*fU*fU*fC*fC | | XXXXXXXXX | | |
| WV- | UCCCCCAGGGC | 796 | fU*fC*fC*fC*fC*fC*mA*mG*mG*mG* | 1548 | XXXXXXXXXX | DMD mouse Exon23 | DMD |
| 4652 | AGGCCAUUC | | mC*mA*mG*mG*fC*fC*fA*fU*fU*fC | | XXXXXXXXX | | |
| WV- | AUCCCCCAGGG | 797 | fA*fU*fC*fC*fC*fC*mC*mA*mG*mG* | 1549 | XXXXXXXXXX | DMD mouse Exon23 | DMD |
| 4653 | CAGGCCAUU | | mG*mC*mA*mG*fG*fC*fC*fA*fU*fU | | XXXXXXXXX | | |
| WV- | CAUCCCCCAGG | 798 | fC*fA*fU*fC*fC*fC*mC*mC*mA*mG* | 1550 | XXXXXXXXXX | DMD mouse Exon23 | DMD |
| 4654 | GCAGGCCAU | | mG*mG*mC*mA*fG*fG*fC*fC*fA*fU | | XXXXXXXXX | | |
| WV- | GCAUCCCCCAG | 799 | fG*fC*fA*fU*fC*fC*mC*mC*mC*mA* | 1551 | XXXXXXXXXX | DMD mouse Exon23 | DMD |
| 4655 | GGCAGGCCA | | mG*mG*mG*mC*fA*fG*fG*fC*fC*fA | | XXXXXXXXX | | |
| WV- | AGCAUCCCCCA | 800 | fA*fG*fC*fA*fU*fC*mC*mC*mC*mC* | 1552 | XXXXXXXXXX | DMD mouse Exon23 | DMD |
| 4656 | GGGCAGGCC | | mA*mG*mG*mG*fC*fA*fG*fG*fC*fC | | XXXXXXXXX | | |
| WV- | CAGCAUCCCCC | 801 | fC*fA*fG*fC*fA*fU*mC*mC*mC*mC* | 1553 | XXXXXXXXXX | DMD mouse Exon23 | DMD |
| 4657 | AGGGCAGGC | | mC*mA*mG*mG*fG*fC*fA*fG*fG*fC | | XXXXXXXXX | | |
| WV- | UCAGCAUCCCC | 802 | fU*fC*fA*fG*fC*fA*mU*mC*mC*mC* | 1554 | XXXXXXXXXX | DMD mouse Exon23 | DMD |
| 4658 | CAGGGCAGG | | mC*mC*mA*mG*fG*fG*fC*fA*fG*fG | | XXXXXXXXX | | |
| WV- | UUCAGCAUCCC | 803 | fU*fU*fC*fA*fG*fC*mA*mU*mC*mC* | 1555 | XXXXXXXXXX | DMD mouse Exon23 | DMD |
| 4659 | CCAGGGCAG | | mC*mC*mC*mA*fG*fG*fG*fC*fA*fG | | XXXXXXXXX | | |
| WV- | UUUCAGCAUCC | 804 | fU*fU*fU*fC*fA*fG*mC*mA*mU*mC* | 1556 | XXXXXXXXXX | DMD mouse Exon23 | DMD |
| 4660 | CCCAGGGCA | | mC*mC*mC*mC*fA*fG*fG*fG*fC*fA | | XXXXXXXXX | | |
| WV- | AUUUCAGCAU | 805 | fA*fU*fU*fU*fC*fA*mG*mC*mA*mU | 1557 | XXXXXXXXXX | DMD mouse Exon23 | DMD |
| 4661 | CCCCCAGGGC | | *mC*mC*mC*mC*fC*fA*fG*fG*fG*fC | | XXXXXXXXX | | |
| WV- | GAUUUCAGCA | 806 | fG*fA*fU*fU*fU*fC*mA*mG*mC*mA | 1558 | XXXXXXXXXX | DMD mouse Exon23 | DMD |
| 4662 | UCCCCCAGGG | | *mU*mC*mC*mC*fC*fC*fA*fG*fG*fG | | XXXXXXXXX | | |
| WV- | GGAUUUCAGC | 807 | fG*fG*fA*fU*fU*fU*mC*mA*mG*mC | 1559 | XXXXXXXXXX | DMD mouse Exon23 | DMD |
| 4663 | AUCCCCCAGG | | *mA*mU*mC*mC*fC*fC*fC*fA*fG*fG | | XXXXXXXXX | | |
| WV- | AGGAUUUCAG | 808 | fA*fG*fG*fA*fU*fU*mU*mC*mA*mG | 1560 | XXXXXXXXXX | DMD mouse Exon23 | DMD |
| 4664 | CAUCCCCCAG | | *mC*mA*mU*mC*fC*fC*fC*fC*fA*fG | | XXXXXXXXX | | |
| WV- | CAGGAUUUCA | 809 | fC*fA*fG*fG*fA*fU*mU*mU*mC*mA | 1561 | XXXXXXXXXX | DMD mouse Exon23 | DMD |
| 4665 | GCAUCCCCCA | | *mG*mC*mA*mU*fC*fC*fC*fC*fC*fA | | XXXXXXXXX | | |
| WV- | UCAGGAUUUC | 810 | fU*fC*fA*fG*fG*fA*mU*mU*mU*mC | 1562 | XXXXXXXXXX | DMD mouse Exon23 | DMD |
| 4666 | AGCAUCCCCC | | *mA*mG*mC*mA*fU*fC*fC*fC*fC*fC | | XXXXXXXXX | | |
| WV- | UUCAGGAUUU | 811 | fU*fU*fC*fA*fG*fG*mA*mU*mU*mU | 1563 | XXXXXXXXXX | DMD mouse Exon23 | DMD |
| 4667 | CAGCAUCCCC | | *mC*mA*mG*mC*fA*fU*fC*fC*fC*fC | | XXXXXXXXX | | |
| WV- | UUUCAGGAUU | 812 | fU*fU*fU*fC*fA*fG*mG*mA*mU*mU | 1564 | XXXXXXXXXX | DMD mouse Exon23 | DMD |
| 4668 | UCAGCAUCCC | | *mU*mC*mA*mG*fC*fA*fU*fC*fC*fC | | XXXXXXXXX | | |
| WV- | UUUUCAGGAU | 813 | fU*fU*fU*fU*fC*fA*mG*mG*mA*mU | 1565 | XXXXXXXXXX | DMD mouse Exon23 | DMD |
| 4669 | UUCAGCAUCC | | *mU*mU*mC*mA*fG*fC*fA*fU*fC*fC | | XXXXXXXXX | | |
| WV- | UUUUUCAGGA | 814 | fU*fU*fU*fU*fU*fC*mA*mG*mG*mA | 1566 | XXXXXXXXXX | DMD mouse Exon23 | DMD |
| 4670 | UUUCAGCAUC | | *mU*mU*mU*mC*fA*fG*fC*fA*fU*fC | | XXXXXXXXX | | |
| WV- | UUUUUUCAGG | 815 | fU*fU*fU*fU*fU*fU*mC*mA*mG*mG | 1567 | XXXXXXXXXX | DMD mouse Exon23 | DMD |
| 4671 | AUUUCAGCAU | | *mA*mU*mU*mU*fC*fA*fG*fC*fA*fU | | XXXXXXXXX | | |
| WV- | GUUUUUUCAG | 816 | fG*fU*fU*fU*fU*fU*mU*mC*mA*mG | 1568 | XXXXXXXXXX | DMD mouse Exon23 | DMD |
| 4672 | GAUUUCAGCA | | *mG*mA*mU*mU*fU*fC*fA*fG*fC*fA | | XXXXXXXXX | | |
| WV- | UGUUUUUUCA | 817 | fU*fG*fU*fU*fU*fU*mU*mU*mC*mA | 1569 | XXXXXXXXXX | DMD mouse Exon23 | DMD |
| 4673 | GGAUUUCAGC | | *mG*mG*mA*mU*fU*fU*fC*fA*fG*fC | | XXXXXXXXX | | |
| WV- | CUGUUUUUUC | 818 | fC*fU*fG*fU*fU*fU*mU*mU*mU*mC | 1570 | XXXXXXXXXX | DMD mouse Exon23 | DMD |
| 4674 | AGGAUUUCAG | | *mA*mG*mG*mA*fU*fU*fU*fC*fA*fG | | XXXXXXXXX | | |
| WV- | GCUGUUUUUU | 819 | fG*fC*fU*fG*fU*fU*mU*mU*mU*mU | 1571 | XXXXXXXXXX | DMD mouse Exon23 | DMD |
| 4675 | CAGGAUUUCA | | *mC*mA*mG*mG*fA*fU*fU*fU*fC*fA | | XXXXXXXXX | | |
| WV- | AGCUGUUUUU | 820 | fA*fG*fC*fU*fG*fU*mU*mU*mU*mU | 1572 | XXXXXXXXXX | DMD mouse Exon23 | DMD |
| 4676 | UCAGGAUUUC | | *mU*mC*mA*mG*fG*fA*fU*fU*fU*fC | | XXXXXXXXX | | |
| WV- | GAGCUGUUUU | 821 | fG*fA*fG*fC*fU*fG*mU*mU*mU*mU | 1573 | XXXXXXXXXX | DMD mouse Exon23 | DMD |
| 4677 | UUCAGGAUUU | | *mU*mU*mC*mA*fG*fG*fA*fU*fU*fU | | XXXXXXXXX | | |
| WV- | UGAGCUGUUU | 822 | fU*fG*fA*fG*fC*fU*mG*mU*mU*mU | 1574 | XXXXXXXXXX | DMD mouse Exon23 | DMD |
| 4678 | UUUCAGGAUU | | *mU*mU*mU*mC*fA*fG*fG*fA*fU*fU | | XXXXXXXXX | | |
| WV- | UUGAGCUGUU | 823 | fU*fU*fG*fA*fG*fC*mU*mG*mU*mU | 1575 | XXXXXXXXXX | DMD mouse Exon23 | DMD |
| 4679 | UUUUCAGGAU | | *mU*mU*mU*mU*fC*fA*fG*fG*fA*fU | | XXXXXXXXX | | |
| WV- | UUUGAGCUGU | 824 | fU*fU*fU*fG*fA*fG*mC*mU*mG*mU | 1576 | XXXXXXXXXX | DMD mouse Exon23 | DMD |
| 4680 | UUUUUCAGGA | | *mU*mU*mU*mU*fU*fC*fA*fG*fG*fA | | XXXXXXXXX | | |
| WV- | GUUUGAGCUG | 825 | fG*fU*fU*fU*fG*fA*mG*mC*mU*mG | 1577 | XXXXXXXXXX | DMD mouse Exon23 | DMD |
| 4681 | UUUUUUCAGG | | *mU*mU*mU*mU*fU*fU*fC*fA*fG*fG | | XXXXXXXXX | | |
| WV- | UUGUUUGAGC | 826 | fU*fU*fG*fU*fU*fU*mG*mA*mG*mC | 1578 | XXXXXXXXXX | DMD mouse Exon23 | DMD |
| 4682 | UGUUUUUUCA | | *mU*mG*mU*mU*fU*fU*fU*fU*fC*fA | | XXXXXXXXX | | |
| WV- | CAUUGUUUGA | 827 | fC*fA*fU*fU*fG*fU*mU*mU*mG*mA | 1579 | XXXXXXXXXX | DMD mouse Exon23 | DMD |
| 4683 | GCUGUUUUUU | | *mG*mC*mU*mG*fU*fU*fU*fU*fU*fU | | XXXXXXXXX | | |
| WV- | GCAUUGUUUG | 828 | fG*fC*fA*fU*fU*fG*mU*mU*mU*mG | 1580 | XXXXXXXXXX | DMD mouse Exon23 | DMD |
| 4684 | AGCUGUUUUU | | *mA*mG*mC*mU*fG*fU*fU*fU*fU*fU | | XXXXXXXXX | | |
| WV- | UGCAUUGUUU | 829 | fU*fG*fC*fA*fU*fU*mG*mU*mU*mU | 1581 | XXXXXXXXXX | DMD mouse Exon23 | DMD |
| 4685 | GAGCUGUUUU | | *mG*mA*mG*mC*fU*fG*fU*fU*fU*fU | | XXXXXXXXX | | |
| WV- | CUGCAUUGUU | 830 | fC*fU*fG*fC*fA*fU*mU*mG*mU*mU | 1582 | XXXXXXXXXX | DMD mouse Exon23 | DMD |
| 4686 | UGAGCUGUUU | | *mU*mG*mA*mG*fC*fU*fG*fU*fU*fU | | XXXXXXXXX | | |
| WV- | UCUGCAUUGU | 831 | fU*fC*fU*fG*fC*fA*mU*mU*mG*mU | 1583 | XXXXXXXXXX | DMD mouse Exon23 | DMD |
| 4687 | UUGAGCUGUU | | *mU*mU*mG*mA*fG*fC*fU*fG*fU*fU | | XXXXXXXXX | | |
| WV- | CUCUGCAUUG | 832 | fC*fU*fC*fU*fG*fC*mA*mU*mU*mG* | 1584 | XXXXXXXXXX | DMD mouse Exon23 | DMD |
| 4688 | UUUGAGCUGU | | mU*mU*mU*mG*fA*fG*fC*fU*fG*fU | | XXXXXXXXX | | |
| WV- | ACUCUGCAUU | 833 | fA*fC*fU*fC*fU*fG*mC*mA*mU*mU* | 1585 | XXXXXXXXXX | DMD mouse Exon23 | DMD |
| 4689 | GUUUGAGCUG | | mG*mU*mU*mU*fG*fA*fG*fC*fU*fG | | XXXXXXXXX | | |
| WV- | UACUCUGCAU | 834 | fU*fA*fC*fU*fC*fU*mG*mC*mA*mU* | 1586 | XXXXXXXXXX | DMD mouse Exon23 | DMD |
| 4690 | UGUUUGAGCU | | mU*mG*mU*mU*fU*fG*fA*fG*fC*fU | | XXXXXXXXX | | |
| WV- | UUACUCUGCA | 835 | fU*fU*fA*fC*fU*fC*mU*mG*mC*mA* | 1587 | XXXXXXXXXX | DMD mouse Exon23 | DMD |
| 4691 | UUGUUUGAGC | | mU*mU*mG*mU*fU*fU*fG*fA*fG*fC | | XXXXXXXXX | | |
| WV- | CUUACUCUGCA | 836 | fC*fU*fU*fA*fC*fU*mC*mU*mG*mC* | 1588 | XXXXXXXXXX | DMD mouse Exon23 | DMD |
| 4692 | UUGUUUGAG | | mA*mU*mU*mG*fU*fU*fU*fG*fA*fG | | XXXXXXXXX | | |
| WV- | UCUUACUCUGC | 837 | fU*fC*fU*fU*fA*fC*mU*mC*mU*mG* | 1589 | XXXXXXXXXX | DMD mouse Exon23 | DMD |
| 4693 | AUUGUUUGA | | mC*mA*mU*mU*fG*fU*fU*fU*fG*fA | | XXXXXXXXX | | |
| WV- | AUCUUACUCU | 838 | fA*fU*fC*fU*fU*fA*mC*mU*mC*mU* | 1590 | XXXXXXXXXX | DMD mouse Exon23 | DMD |
| 4694 | GCAUUGUUUG | | mG*mC*mA*mU*fU*fG*fU*fU*fU*fG | | XXXXXXXXX | | |
| WV- | AAUCUUACUC | 839 | fA*fA*fU*fC*fU*fU*mA*mC*mU*mC* | 1591 | XXXXXXXXXX | DMD mouse Exon23 | DMD |
| 4695 | UGCAUUGUUU | | mU*mG*mC*mA*fU*fU*fG*fU*fU*fU | | XXXXXXXXX | | |
| WV- | CAAAUCUUAC | 840 | fC*fA*fA*fA*fU*fC*mU*mU*mA*mC* | 1592 | XXXXXXXXXX | DMD mouse Exon23 | DMD |
| 4696 | UCUGCAUUGU | | mU*mC*mU*mG*fC*fA*fU*fU*fG*fU | | XXXXXXXXX | | |
| WV- | GAUACAAAUC | 841 | fG*fA*fU*fA*fC*fA*mA*mA*mU*mC | 1593 | XXXXXXXXXX | DMD mouse Exon23 | DMD |
| 4697 | UUACUCUGCA | | *mU*mU*mA*mC*fU*fC*fU*fG*fC*fA | | XXXXXXXXX | | |
| WV- | GGGUCAGCT | 842 | mG*mG*mG*mU*mC*A*G*C*T*G* | 1594 | XXXXXXXXX | ASO1 Malat1 2OMe | Malat1 |
| 2559 | GCCAATGCU | | C*C*A*A*T*mG*mC*mU*mA*mG | | XXXXXXXXXX | 5-10-5 Full PS version | |
| | AG | | | | | | |
| WV- | GGGUCAGCT | 843 | mG*mGmGmUmC*A*G*C*T*G*C*C | 1595 | XOOOXXXXX | ASO1 Malat1 2OMe | Malat1 |
| 2560 | GCCAATGCU | | *A*A*T*mGmCmUmA*mG | | XXXXXXOOOX | 5-10-5 WV-1497 like | |
| | AG | | | | | version | |
| WV- | GGGUCAGCT | 844 | mG*G*mG*mU*mC*A*G*C*T*G*C* | 1596 | XXXXXXXXX | ASO1 Malat1 2OMe | Malat1 |
| 2562 | GCCAATGCU | | C*A*A*T*mG*mC*mU*A*mG | | XXXXXXXXXX | 1-1-3-10-3-1-1 Full PS | |
| | AG | | | | | version Frank2 | |
| WV- | GGGUCAGCT | 845 | mG*G*mGmUmC*A*G*C*T*G*C*C | 1597 | XXOOXXXXX | ASO1 Malat1 2OMe | Malat1 |
| 2564 | GCCAATGCU | | *A*A*T*mGmCmUA*mG | | XXXXXXOOOX | 1-1-3-10-3-1-1 PO PS | |
| | AG | | | | | Frank2 | |
| WV- | GGGUCAGCT | 846 | mG*mG*G*mU*mC*A*G*C*T*G*C* | 1598 | XXXXXXXXX | ASO1 Malat1 2OMe | Malat1 |
| 2566 | GCCAATGCTAG | | C*A*A*T*mG*mC*T*mA*mG | | XXXXXXXXXX | 2-1-2-10-2-1-2 Full PS | |
| | | | | | | version Frank3 | |
| WV- | GGGUCAGCT | 847 | mG*mGG*mUmC*A*G*C*T*G*C*C | 1599 | XOXOXXXXX | ASO1 Malat1 2OMe | Malat1 |
| 2568 | GCCAATGCTAG | | *A*A*T*mGmCT*mA*mG | | XXXXXXOOXX | 2-1-2-10-2-1-2 PO PS | |
| | | | | | | version Frank3 | |
| WV- | GGGTCAGCTG | 848 | mG*mG*mG*T*mC*A*G*C*T*G*C* | 1600 | XXXXXXXXX | ASO1 Malat1 2OMe | Malat1 |
| 2570 | CCAATGCUAG | | C*A*A*T*mG*C*mU*mA*mG | | XXXXXXXXXX | 3-1-1-10-1-1-3 Full PS | |
| | | | | | | version Nenad1 | |
| WV- | GGGTCAGCTG | 849 | mG*mGmGT*mC*A*G*C*T*G*C*C* | 1601 | XOOXXXXXX | ASO1 Malat1 2OMe | Malat1 |
| 2572 | CCAATGCUAG | | A*A*T*mGC*mUmA*mG | | XXXXXXOXOX | 3-1-1-10-1-1-3 PO PS | |
| | | | | | | like version Nenad1 | |
| WV- | GGGUCAGCT | 850 | G*G*mG*mU*mC*A*G*C*T*G*C*C | 1602 | XXXXXXXXX | ASO1 Malat1 2OMe | Malat1 |
| 2574 | GCCAATGCU | | *A*A*T*mG*mC*mU*A*G | | XXXXXXXXX | 2-3-10-3-2 PO PS like | |
| | AG | | | | | version Chandra1 | |
| WV- | GGGUCAGCT | 851 | G*mGmGmUmC*A*G*C*T*G*C*C* | 1603 | XOOOXXXXX | ASO1 Malat1 2OMe | Malat1 |
| 2576 | GCCAATGCU | | A*A*T*mGmCmUmA*G | | XXXXXXOOOX | 1-4-10-4-1 PO PS like | |
| | AG | | | | | version Chandra2 | |
| WV- | GGGTCAGCTG | 852 | Geo*Geo*Geo*Teo*m5Ceo*A*G*C*T | 1604 | XXXXXXXXX | Randomer for WV- | Malat1 |
| 2735 | CCAATGCTAG | | *G*C*C*A*A*T*Geo*m5Ceo*Teo*A | | XXXXXXXXXX | 2526 | |
| | | | eo*Geo | | | | |
| WV- | GGGTCAGCTG | 853 | Geo*Geo*Geo*Teo*Ceo*A*G*C*T*G | 1605 | XXXXXXXXX | Randomer for WV- | Malat1 |
| 2736 | CCAATGCTAG | | *C*C*A*A*T*Geo*Ceo*Teo*Aeo*Geo | | XXXXXXXXXX | 2526 | |
| WV- | GGGUCAGCT | 854 | Mod013L001*mG*mG*mG*mU*mC* | 1606 | OXXXXXXXX | Laurie acid OMe full- | Malat1 |
| 2753 | GCCAATGCU | | A*G*C*T*G*C*C*A*A*T*mG*mC* | | XXXXXXXXX | PS | |
| | AG | | mU*mA*mG | | XXX | | |
| WV- | GGGUCAGCT | 855 | Mod014L001*mG*mG*mG*mU*mC* | 1607 | OXXXXXXXX | Myristic acid OMe | Malat1 |
| 2754 | GCCAATGCU | | A*G*C*T*G*C*C*A*A*T*mG*mC* | | XXXXXXXXX | full-PS | |
| | AG | | mU*mA*mG | | XXX | | |
| WV- | GGGUCAGCT | 856 | Mod005L001*mG*mG*mG*mU*mC* | 1608 | OXXXXXXXX | Palmitic acid OMe | Malat1 |
| 2755 | GCCAATGCU | | A*G*C*T*G*C*C*A*A*T*mG*mC* | | XXXXXXXXX | full-PS | |
| | AG | | mU*mA*mG | | XXX | | |
| WV- | GGGUCAGCT | 857 | Mod015L001*mG*mG*mG*mU*mC* | 1609 | OXXXXXXXX | Stearic acid OMe full- | Malat1 |
| 2756 | GCCAATGCU | | A*G*C*T*G*C*C*A*A*T*mG*mC* | | XXXXXXXXX | PS | |
| | AG | | mU*mA*mG | | XXX | | |
| WV- | GGGUCAGCT | 858 | Mod016L001*mG*mG*mG*mU*mC* | 1610 | OXXXXXXXX | Oleic acid OMe full- | Malat1 |
| 2757 | GCCAATGCU | | A*G*C*T*G*C*C*A*A*T*mG*mC* | | XXXXXXXXX | PS | |
| | AG | | mU*mA*mG | | XXX | | |
| WV- | GGGUCAGCT | 859 | Mod017L001*mG*mG*mG*mU*mC* | 1611 | OXXXXXXXX | Linoleic acid OMe | Malat1 |
| 2758 | GCCAATGCU | | A*G*C*T*G*C*C*A*A*T*mG*mC* | | XXXXXXXXX | full-PS | |
| | AG | | mU*mA*mG | | XXX | | |
| WV- | GGGUCAGCT | 860 | Mod018L001*mG*mG*mG*mU*mC* | 1612 | OXXXXXXXX | alpha-Linolenic acid | Malat1 |
| 2759 | GCCAATGCU | | A*G*C*T*G*C*C*A*A*T*mG*mC* | | XXXXXXXXX | OMe full-PS | |
| | AG | | mU*mA*mG | | XXX | | |
| WV- | GGGUCAGCT | 861 | Mod019L001*mG*mG*mG*mU*mC* | 1613 | OXXXXXXXX | gamma-Linolenic acid | Malat1 |
| 2760 | GCCAATGCU | | A*G*C*T*G*C*C*A*A*T*mG*mC* | | XXXXXXXXX | OMe full-PS | |
| | AG | | mU*mA*mG | | XXX | | |
| WV- | GGGUCAGCT | 862 | Mod006L001*mG*mG*mG*mU*mC* | 1614 | OXXXXXXXX | DHA OMe full-PS | Malat1 |
| 2761 | GCCAATGCU | | A*G*C*T*G*C*C*A*A*T*mG*mC* | | XXXXXXXXX | | |
| | AG | | mU*mA*mG | | XXX | | |
| WV- | GGGUCAGCT | 863 | Mod020L001*mG*mG*mG*mU*mC* | 1615 | OXXXXXXXX | Turbinaric acid OMe | Malat1 |
| 2762 | GCCAATGCU | | A*G*C*T*G*C*C*A*A*T*mG*mC* | | XXXXXXXXX | full-PS | |
| | AG | | mU*mA*mG | | XXX | | |
| WV- | GGGUCAGCT | 864 | Mod021*mG*mG*mG*mU*mC*A*G* | 1616 | XXXXXXXXX | Dilinoleyl alcohol | Malat1 |
| 2763 | GCCAATGCU | | C*T*G*C*C*A*A*T*mG*mC*mU*m | | XXXXXXXXX | OMe full-PS | |
| | AG | | A*mG | | XX | | |
| WV- | GGGUCAGCT | 865 | Mod024L001*mG*mG*mG*mU*mC* | 1617 | XXXXXXXXX | Triantennary GlcNAc | Malat1 |
| 2764 | GCCAATGCU | | A*G*C*T*G*C*C*A*A*T*mG*mC* | | XXXXXXXXX | OMe full-PS | |
| | AG | | mU*mA*mG | | XX | | |
| WV- | GGGUCAGCT | 866 | Mod025L001*mG*mG*mG*mU*mC* | 1618 | XXXXXXXXX | Triantennary beta- | Malat1 |
| 2765 | GCCAATGCU | | A*G*C*T*G*C*C*A*A*T*mG*mC* | | XXXXXXXXX | Mannose OMe full-PS | |
| | AG | | mU*mA*mG | | XX | | |
| WV- | GGGUCAGCT | 867 | Mod026L001*mG*mG*mG*mU*mC* | 1619 | XXXXXXXXX | Triantennary alpha- | Malat1 |
| 2766 | GCCAATGCU | | A*G*C*T*G*C*C*A*A*T*mG*mC* | | XXXXXXXXX | Mannose OMe full-PS | |
| | AG | | mU*mA*mG | | XX | | |
| WV- | GGGUCAGCT | 868 | Mod013L001*mG*mGmGmUmC*A* | 1620 | OXXOOOXXX | Laurie acid OMe | Malat1 |
| 2767 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 869 | Mod014L001*mG*mGmGmUmC*A* | 1621 | OXXOOOXXX | Myristic acid OMe | Malat1 |
| 2768 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 870 | Mod005L001*mG*mGmGmUmC*A* | 1622 | OXXOOOXXX | Palmitic acid OMe | Malat1 |
| 2769 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 871 | Mod015L001*mG*mGmGmUmC*A* | 1623 | OXXOOOXXX | Stearic acid OMe | Malat1 |
| 2770 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 872 | Mod016L001*mG*mGmGmUmC*A* | 1624 | OXXOOOXXX | Oleic acid OMe | Malat1 |
| 2771 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 873 | Mod017L001*mG*mGmGmUmC*A* | 1625 | OXXOOOXXX | Linoleic acid OMe | Malat1 |
| 2772 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 874 | Mod018L001*mG*mGmGmUmC*A* | 1626 | OXXOOOXXX | alpha-Linolenic acid | Malat1 |
| 2773 | GCCAATGCU | | G*C*T*G*C*C*A*A*T*mGmCmUm | | XXXXXXXXO | OMe PS\/PO wing | |
| | AG | | A*mG | | OOX | | |
| WV- | GGGUCAGCT | 875 | Mod019L001*mG*mGmGmUmC*A* | 1627 | OXXOOOXXX | gamma-Linolenic acid | Malat1 |
| 2774 | GCCAATGCU | | G*C*T*G*C*C*A*A*T*mGmCmUm | | XXXXXXXXO | OMe PS\/PO wing | |
| | AG | | A*mG | | OOX | | |
| WV- | GGGUCAGCT | 876 | Mod006L001*mG*mGmGmUmC*A* | 1628 | OXXOOOXXX | DHA OMe PS\/PO | Malat1 |
| 2775 | GCCAATGCU | | G*C*T*G*C*C*A*A*T*mGmCmUm | | XXXXXXXXO | wing | |
| | AG | | A*mG | | OOX | | |
| WV- | GGGUCAGCT | 877 | Mod020L001*mG*mGmGmUmC*A* | 1629 | OXXOOOXXX | Turbinaric acid OMe | Malat1 |
| 2776 | GCCAATGCU | | G*C*T*G*C*C*A*A*T*mGmCmUm | | XXXXXXXXO | PS\/PO wing | |
| | AG | | A*mG | | OOX | | |
| WV- | GGGUCAGCT | 878 | Mod021*mG*mGmGmUmC*A*G*C* | 1630 | XXOOOXXXX | Dilinoleyl alcohol | Malat1 |
| 2777 | GCCAATGCU | | T*G*C*C*A*A*T*mGmCmUmA*mG | | XXXXXXXOO | OMe PS\/PO wing | |
| | AG | | | | | OX | |
| WV- | GGGUCAGCT | 879 | Mod024L001*mG*mGmGmUmC*A* | 1631 | XXOOOXXXX | Triantennary GlcNAc | Malat1 |
| 2778 | GCCAATGCU | | G*C*T*G*C*C*A*A*T*mGmCmUm | | XXXXXXXOO | OMe PS\/PO wing | |
| | AG | | A*mG | | OX | | |
| WV- | GGGUCAGCT | 880 | Mod025L001*mG*mGmGmUmC*A* | 1632 | XXOOOXXXX | Triantennary beta- | Malat1 |
| 2779 | GCCAATGCU | | G*C*T*G*C*C*A*A*T*mGmCmUm | | XXXXXXXOO | Mannose OMe PS\/PO | |
| | AG | | A*mG | | OX | wing | |
| WV- | GGGUCAGCT | 881 | Mod026L001*mG*mGmGmUmC*A* | 1633 | XXOOOXXXX | Triantennary alpha- | Malat1 |
| 2780 | GCCAATGCU | | G*C*T*G*C*C*A*A*T*mGmCmUm | | XXXXXXXOO | Mannose OMe PS\/PO | |
| | AG | | A*mG | | OX | wing | |
| WV- | CUAGCAUUG | 882 | rCrUrArGrCrArUrUrGrGrCrArGrCrUr | 1634 | OOOOOOOOO | complementary RNA | Malat1 |
| 2781 | GCAGCUGAC | | GrArCrCrC | | OOOOOOOOOO | coding Malat1 | |
| | CC | | | | | | |
| WV- | GGGTCAGCTG | 883 | L001*Geo*Geo*Geo*Teo*m5Ceo*A* | 1635 | XXXXXXXXX | C6amine linker MOE | Malat1 |
| 2809 | CCAATGCTAG | | G*C*T*G*C*C*A*A*T*Geo*m5Ceo* | | XXXXXXXXX | full-PS | |
| | | | Teo*Aeo*Geo | | XX | | |
| WV- | GGGUCAGCT | 884 | L001*mG*mG*mG*mU*mC*A*G*C* | 1636 | XXXXXXXXX | C6amine linker OMe | Malat1 |
| 2810 | GCCAATGCU | | T*G*C*C*A*A*T*mG*mC*mU*mA* | | XXXXXXXXX | full-PS | |
| | AG | | mG | | XX | | |
| WV- | GGGUCAGCT | 885 | L001*mG*mGmGmUmC*A*G*C*T* | 1637 | XXOOOXXXX | C6amine linker OMe | Malat1 |
| 2811 | GCCAATGCU | | G*C*C*A*A*T*mGmCmUmA*mG | | XXXXXXXOO | PS\/PO wing | |
| | AG | | | | OX | | |
| WV- | GGGTCAGCTG | 886 | Mod013L001*Geo*Geo*Geo*Teo*m5 | 1638 | OXXXXXXXX | Lauric acid MOE full- | Malat1 |
| 2821 | CCAATGCTAG | | Ceo*A*G*C*T*G*C*C*A*A*T*Geo* | | XXXXXXXXX | PS | |
| | | | m5Ceo*Teo*Aeo*Geo | | XXX | | |
| WV- | GGGTCAGCTG | 887 | Mod014L001*Geo*Geo*Geo*Teo*m5 | 1639 | OXXXXXXXX | Myristic acid MOE | Malat1 |
| 2822 | CCAATGCTAG | | Ceo*A*G*C*T*G*C*C*A*A*T*Geo* | | XXXXXXXXX | full-PS | |
| | | | m5Ceo*Teo*Aeo*Geo | | XXX | | |
| WV- | GGGTCAGCTG | 888 | Mod005L001*Geo*Geo*Geo*Teo*m5 | 1640 | OXXXXXXXX | Palmitic acid MOE | Malat1 |
| 2823 | CCAATGCTAG | | Ceo*A*G*C*T*G*C*C*A*A*T*Geo* | | XXXXXXXXX | full-PS | |
| | | | m5Ceo*Teo*Aeo*Geo | | XXX | | |
| WV- | GGGTCAGCTG | 889 | Mod015L001*Geo*Geo*Geo*Teo*m5 | 1641 | OXXXXXXXX | Stearic acid MOE full- | Malat1 |
| 2824 | CCAATGCTAG | | Ceo*A*G*C*T*G*C*C*A*A*T*Geo* | | XXXXXXXXX | PS | |
| | | | m5Ceo*Teo*Aeo*Geo | | XXX | | |
| WV- | GGGTCAGCTG | 890 | Mod016L001*Geo*Geo*Geo*Teo*m5 | 1642 | OXXXXXXXX | Oleic acid MOE full- | Malat1 |
| 2825 | CCAATGCTAG | | Ceo*A*G*C*T*G*C*C*A*A*T*Geo* | | XXXXXXXXX | PS | |
| | | | m5Ceo*Teo*Aeo*Geo | | XXX | | |
| WV- | GGGTCAGCTG | 891 | Mod017L001*Geo*Geo*Geo*Teo*m5 | 1643 | OXXXXXXXX | Linoleic acid MOE | Malat1 |
| 2826 | CCAATGCTAG | | Ceo*A*G*C*T*G*C*C*A*A*T*Geo* | | XXXXXXXXX | full-PS | |
| | | | m5Ceo*Teo*Aeo*Geo | | XXX | | |
| WV- | GGGTCAGCTG | 892 | Mod018L001*Geo*Geo*Geo*Teo*m5 | 1644 | OXXXXXXXX | alpha-Linolenic acid | Malat1 |
| 2827 | CCAATGCTAG | | Ceo*A*G*C*T*G*C*C*A*A*T*Geo* | | XXXXXXXXX | MOE full-PS | |
| | | | m5Ceo*Teo*Aeo*Geo | | XXX | | |
| WV- | GGGTCAGCTG | 893 | Mod019L001*Geo*Geo*Geo*Teo*m5 | 1645 | OXXXXXXXX | gamma-Linolenic acid | Malat1 |
| 2828 | CCAATGCTAG | | Ceo*A*G*C*T*G*C*C*A*A*T*Geo* | | XXXXXXXXX | MOE full-PS | |
| | | | m5Ceo*Teo*Aeo*Geo | | XXX | | |
| WV- | GGGTCAGCTG | 894 | Mod006L001*Geo*Geo*Geo*Teo*m5 | 1646 | OXXXXXXXX | DHA MOE full-PS | Malat1 |
| 2829 | CCAATGCTAG | | Ceo*A*G*C*T*G*C*C*A*A*T*Geo* | | XXXXXXXXX | | |
| | | | m5Ceo*Teo*Aeo*Geo | | XXX | | |
| WV- | GGGTCAGCTG | 895 | Mod020L001*Geo*Geo*Geo*Teo*m5 | 1647 | OXXXXXXXX | Turbinaric acid MOE | Malat1 |
| 2830 | CCAATGCTAG | | Ceo*A*G*C*T*G*C*C*A*A*T*Geo* | | XXXXXXXXX | full-PS | |
| | | | m5Ceo*Teo*Aeo*Geo | | XXX | | |
| WV- | GGGTCAGCTG | 896 | Mod021*Geo*Geo*Geo*Teo*m5Ceo* | 1648 | XXXXXXXXX | Dilinoleyl alcohol | Malat1 |
| 2831 | CCAATGCTAG | | A*G*C*T*G*C*C*A*A*T*Geo*m5C | | XXXXXXXXX | MOE full-PS | |
| | | | eo*Teo*Aeo*Geo | | XX | | |
| WV- | GGGTCAGCTG | 897 | Mod024L001*Geo*Geo*Geo*Teo*m5 | 1649 | XXXXXXXXX | Triantennary GlcNAc | Malat1 |
| 2832 | CCAATGCTAG | | Ceo*A*G*C*T*G*C*C*A*A*T*Geo* | | XXXXXXXXX | MOE full-PS | |
| | | | m5Ceo*Teo*Aeo*Geo | | XX | | |
| WV- | GGGTCAGCTG | 898 | Mod025L001*Geo*Geo*Geo*Teo*m5 | 1650 | XXXXXXXXX | Triantennary beta- | Malat1 |
| 2833 | CCAATGCTAG | | Ceo*A*G*C*T*G*C*C*A*A*T*Geo* | | XXXXXXXXX | Mannose MOE full-PS | |
| | | | m5Ceo*Teo*Aeo*Geo | | XX | | |
| WV- | GGGTCAGCTG | 899 | Mod026L001*Geo*Geo*Geo*Teo*m5 | 1651 | XXXXXXXXX | Triantennary alpha- | Malat1 |
| 2834 | CCAATGCTAG | | Ceo*A*G*C*T*G*C*C*A*A*T*Geo* | | XXXXXXXXX | Mannose MOE full-PS | |
| | | | m5Ceo*Teo*Aeo*Geo | | XX | | |
| WV- | GGGTCAGCTG | 900 | Mod027L001*Geo*Geo*Geo*Teo*m5 | 1652 | XXXXXXXXX | sulfonamide MOE | Malat1 |
| 2835 | CCAATGCTAG | | Ceo*A*G*C*T*G*C*C*A*A*T*Geo* | | XXXXXXXXX | full-PS | |
| | | | m5Ceo*Teo*Aeo*Geo | | XX | | |
| WV- | GGGTCAGCTG | 901 | Mod028L001*Geo*Geo*Geo*Teo*m5 | 1653 | XXXXXXXXX | sulfonamide | Malat1 |
| 2836 | CCAATGCTAG | | Ceo*A*G*C*T*G*C*C*A*A*T*Geo* | | XXXXXXXXX | alkylchain MOE full- | |
| | | | m5Ceo*Teo*Aeo*Geo | | XX | PS | |
| WV- | GGGTCAGCTG | 902 | Mod015L001*Geo*Geo*Geo*Teo*m5 | 1654 | OXXXXXXXX | Stearic acid and | Malat1 |
| 3062 | CCAATGCTAG | | Ceo*A*G*C*T*G*C*C*A*A*T*Geo* | | XXXXXXXXX | GlucNAc, MOE, full- | |
| | | | m5Ceo*Teo*Aeo*Geo*L004Mod024 | | XXX | PS | |
| WV- | GGGTCAGCTG | 903 | Mod019L001*Geo*Geo*Geo*Teo*m5 | 1655 | OXXXXXXXX | gamma-Linolenic acid | Malat1 |
| 3063 | CCAATGCTAG | | Ceo*A*G*C*T*G*C*C*A*A*T*Geo* | | XXXXXXXXX | and GlucNAc, MOE, | |
| | | | m5Ceo*Teo*Aeo*Geo*L004Mod024 | | XXX | full-PS | |
| WV- | GGGTCAGCTG | 904 | Mod020L001*Geo*Geo*Geo*Teo*m5 | 1656 | OXXXXXXXX | Turbinaric acid and | Malat1 |
| 3064 | CCAATGCTAG | | Ceo*A*G*C*T*G*C*C*A*A*T*Geo* | | XXXXXXXXX | GlucNAc, MOE, full- | |
| | | | m5Ceo*Teo*Aeo*Geo*L004Mod024 | | XXX | PS | |
| WV- | GGGTCAGCTG | 905 | Mod015L001*Geo*Geo*Geo*Teo*m5 | 1657 | OXXXXXXXX | Stearic acid and | Malat1 |
| 3065 | CCAATGCTAG | | Ceo*A*G*C*T*G*C*C*A*A*T*Geo* | | XXXXXXXXX | Mannose, MOE, full- | |
| | | | m5Ceo*Teo*Aeo*Geo*L004Mod026 | | XXX | PS | |
| WV- | GGGTCAGCTG | 906 | Mod019L001*Geo*Geo*Geo*Teo*m5 | 1658 | OXXXXXXXX | gamma-Linolenic acid | Malat1 |
| 3066 | CCAATGCTAG | | Ceo*A*G*C*T*G*C*C*A*A*T*Geo* | | XXXXXXXXX | and Mannose, MOE, | |
| | | | m5Ceo*Teo*Aeo*Geo*L004Mod026 | | XXX | full-PS | |
| WV- | GGGTCAGCTG | 907 | Mod020L001*Geo*Geo*Geo*Teo*m5 | 1659 | OXXXXXXXX | Turbinaric acid and | Malat1 |
| 3067 | CCAATGCTAG | | Ceo*A*G*C*T*G*C*C*A*A*T*Geo* | | XXXXXXXXX | Mannose, MOE, full- | |
| | | | m5Ceo*Teo*Aeo*Geo*L004Mod026 | | XXX | PS | |
| WV- | UAGCGCCCA | 908 | mU*mA*mG*mC*mG*C*C*C*A*C* | 1660 | XXXXXXXXX | 20mers, Full PS, 5-10- | Malat1 |
| 3154 | CCTCACCCCUC | | C*T*C*A*C*mC*mC*mC*mU*mC | | XXXXXXXXXX | 5 2′OMe gapmers | |
| WV- | UUAGCGCCC | 909 | mU*mU*mA*mG*mC*G*C*C*C*A* | 1661 | XXXXXXXXX | 20mers, Full PS, 5-10- | Malat1 |
| 3155 | ACCTCACCCCU | | C*C*T*C*A*mC*mC*mC*mC*mU | | XXXXXXXXXX | 5 2′OMe gapmers | |
| WV- | CUUAGCGCC | 910 | mC*mU*mU*mA*mG*C*G*C*C*C* | 1662 | XXXXXXXXX | 20mers, Full PS, 5-10- | Malat1 |
| 3156 | CACCTCACCCC | | A*C*C*T*C*mA*mC*mC*mC*mC | | XXXXXXXXXX | 5 2′OMe gapmers | |
| WV- | ACCCCGTCCT | 911 | mA*mC*mC*mC*mC*G*T*C*C*T*G | 1663 | XXXXXXXXX | 20mers, Full PS, 5-10- | Malat1 |
| 3157 | GGAAACCAGG | | *G*A*A*A*mC*mC*mA*mG*mG | | XXXXXXXXXX | 5 2′OMe gapmers | |
| WV- | CCCCGTCCTG | 912 | mC*mC*mC*mC*mG*T*C*C*T*G*G | 1664 | XXXXXXXXX | 20mers, Full PS, 5-10- | Malat1 |
| 3158 | GAAACCAGGA | | *A*A*A*C*mC*mA*mG*mG*mA | | XXXXXXXXXX | 5 2′OMe gapmers | |
| WV- | GCUUAGCGC | 913 | mG*mC*mU*mU*mA*G*C*G*C*C* | 1665 | XXXXXXXXX | 20mers, Full PS, 5-10- | Malat1 |
| 3159 | CCACCTCACCC | | C*A*C*C*T*mC*mA*mC*mC*mC | | XXXXXXXXXX | 5 2′OMe gapmers | |
| WV- | GGCUUAGCG | 914 | mG*mG*mC*mU*mU*A*G*C*G*C* | 1666 | XXXXXXXXX | 20mers, Full PS, 5-10- | Malat1 |
| 3160 | CCCACCUCACC | | C*C*A*C*C*mU*mC*mA*mC*mC | | XXXXXXXXXX | 5 2′OMe gapmers | |
| WV- | CCCGUCCTGG | 915 | mC*mC*mC*mG*mU*C*C*T*G*G* | 1667 | XXXXXXXXX | 20mers, Full PS, 5-10- | Malat1 |
| 3161 | AAACCAGGAG | | A*A*A*C*C*mA*mG*mG*mA*mG | | XXXXXXXXXX | 5 2′OMe gapmers | |
| WV- | UGAACCCCGT | 916 | mU*mG*mA*mA*mC*C*C*C*G*T* | 1668 | XXXXXXXXX | 20mers, Full PS, 5-10- | Malat1 |
| 3162 | CCTGGAAACC | | C*C*T*G*G*mA*mA*mA*mC*mC | | XXXXXXXXXX | 5 2′OMe gapmers | |
| WV- | UUUCCCCTCC | 917 | mU*mU*mU*mC*mC*C*C*T*C*C*C | 1669 | XXXXXXXXX | 20mers, Full PS, 5-10- | Malat1 |
| 3163 | CTCATCAACA | | *T*C*A*T*mC*mA*mA*mC*mA | | XXXXXXXXXX | 5 2′OMe gapmers | |
| WV- | AGCUCCAGTC | 918 | mA*mG*mC*mU*mC*C*A*G*T*C* | 1670 | XXXXXXXXX | 20mers, Full PS, 5-10- | Malat1 |
| 3164 | CCTGAAGGUG | | C*C*T*G*A*mA*mG*mG*mU*mG | | XXXXXXXXXX | 5 2′OMe gapmers | |
| WV- | AGGCUTAGC | 919 | mA*mG*mG*mC*mU*T*A*G*C*G* | 1671 | XXXXXXXXX | 20mers, Full PS, 5-10- | Malat1 |
| 3165 | GCCCACCUCAC | | C*C*C*A*C*mC*mU*mC*mA*mC | | XXXXXXXXXX | 5 2′OMe gapmers | |
| WV- | GUUUCCCCTC | 920 | mG*mU*mU*mU*mC*C*C*C*T*C* | 1672 | XXXXXXXXX | 20mers, Full PS, 5-10- | Malat1 |
| 3166 | CCTCAUCAAC | | C*C*T*C*A*mU*mC*mA*mA*mC | | XXXXXXXXXX | 5 2′OMe gapmers | |
| WV- | AACCCCGTCC | 921 | mA*mA*mC*mC*mC*C*G*T*C*C*T | 1673 | XXXXXXXXX | 20mers, Full PS, 5-10- | Malat1 |
| 3167 | TGGAAACCAG | | *G*G*A*A*mA*mC*mC*mA*mG | | XXXXXXXXXX | 5 2′OMe gapmers | |
| WV- | GAACCCCGTC | 922 | mG*mA*mA*mC*mC*C*C*G*T*C* | 1674 | XXXXXXXXX | 20mers, Full PS, 5-10- | Malat1 |
| 3168 | CTGGAAACCA | | C*T*G*G*A*mA*mA*mC*mC*mA | | XXXXXXXXXX | 5 2′OMe gapmers | |
| WV- | GCUCCAGTCC | 923 | mG*mC*mU*mC*mC*A*G*T*C*C* | 1675 | XXXXXXXXX | 20mers, Full PS, 5-10- | Malat1 |
| 3169 | CTGAAGGUGU | | C*T*G*A*A*mG*mG*mU*mG*mU | | XXXXXXXXXX | 5 2′OMe gapmers | |
| WV- | UUGAACCCC | 924 | mU*mU*mG*mA*mA*C*C*C*C*G* | 1676 | XXXXXXXXX | 20mers, Full PS, 5-10- | Malat1 |
| 3170 | GTCCTGGAAAC | | T*C*C*T*G*mG*mA*mA*mA*mC | | XXXXXXXXXX | 5 2′OMe gapmers | |
| WV- | UUCCCCTCCC | 925 | mU*mU*mC*mC*mC*C*T*C*C*C*T | 1677 | XXXXXXXXX | 20mers, Full PS, 5-10- | Malat1 |
| 3171 | TCATCAACAA | | *C*A*T*C*mA*mA*mC*mA*mA | | XXXXXXXXXX | 5 2′OMe gapmers | |
| WV- | CCGUCCTGGA | 926 | mC*mC*mG*mU*mC*C*T*G*G*A* | 1678 | XXXXXXXXX | 20mers, Full PS, 5-10- | Malat1 |
| 3172 | AACCAGGAGU | | A*A*C*C*A*mG*mG*mA*mG*mU | | XXXXXXXXXX | 5 2′OMe gapmers | |
| WV- | GCAGCTCCAG | 927 | mG*mC*mA*mG*mC*T*C*C*A*G* | 1679 | XXXXXXXXX | 20mers, Full PS, 5-10- | Malat1 |
| 3173 | TCCCTGAAGG | | T*C*C*C*T*mG*mA*mA*mG*mG | | XXXXXXXXXX | 5 2′OMe gapmers | |
| WV- | UGCCAGGCT | 928 | mU*mG*mC*mC*mA*G*G*C*T*G* | 1680 | XXXXXXXXX | 20mers, Full PS, 5-10- | Malat1 |
| 3174 | GGTTATGACUC | | G*T*T*A*T*mG*mA*mC*mU*mC | | XXXXXXXXXX | 5 2′OMe gapmers | |
| WV- | CGUCCTGGA | 929 | mC*mG*mU*mC*mC*T*G*G*A*A* | 1681 | XXXXXXXXX | 20mers, Full PS, 5-10- | Malat1 |
| 3175 | AACCAGGAG | | A*C*C*A*G*mG*mA*mG*mU*mG | | XXXXXXXXXX | 5 2′OMe gapmers | |
| | UG | | | | | | |
| WV- | CAGCUCCAGT | 930 | mC*mA*mG*mC*mU*C*C*A*G*T* | 1682 | XXXXXXXXX | 20mers, Full PS, 5-10- | Malat1 |
| 3176 | CCCTGAAGGU | | C*C*C*T*G*mA*mA*mG*mG*mU | | XXXXXXXXXX | 5 2′OMe gapmers | |
| WV- | CUGCCAGGCT | 931 | mC*mU*mG*mC*mC*A*G*G*C*T* | 1683 | XXXXXXXXX | 20mers, Full PS, 5-10- | Malat1 |
| 3177 | GGTTAUGACU | | G*G*T*T*A*mU*mG*mA*mC*mU | | XXXXXXXXXX | 5 2′OMe gapmers | |
| WV- | UCCUGGAAA | 932 | mU*mC*mC*mU*mG*G*A*A*A*C* | 1684 | XXXXXXXXX | 20mers, Full PS, 5-10- | Malat1 |
| 3178 | CCAGGAGUG | | C*A*G*G*A*mG*mU*mG*mC*mC | | XXXXXXXXXX | 5 2′OMe gapmers | |
| | CC | | | | | | |
| WV- | AAGGCTTAGC | 933 | mA*mA*mG*mG*mC*T*T*A*G*C* | 1685 | XXXXXXXXX | 20mers, Full PS, 5-10- | Malat1 |
| 3179 | GCCCACCUCA | | G*C*C*C*A*mC*mC*mU*mC*mA | | XXXXXXXXXX | 5 2′OMe gapmers | |
| WV- | CCAGGCTGGT | 934 | mC*mC*mA*mG*mG*C*T*G*G*T*T | 1686 | XXXXXXXXX | 20mers, Full PS, 5-10- | Malat1 |
| 3180 | TATGACUCAG | | *A*T*G*A*mC*mU*mC*mA*mG | | XXXXXXXXXX | 5 2′OMe gapmers | |
| WV- | CCUGGAAAC | 935 | mC*mC*mU*mG*mG*A*A*A*C*C* | 1687 | XXXXXXXXX | 20mers, Full PS, 5-10- | Malat1 |
| 3181 | CAGGAGUGC | | A*G*G*A*G*mU*mG*mC*mC*mA | | XXXXXXXXXX | 5 2′OMe gapmers | |
| | CA | | | | | | |
| WV- | GCCAGGCTG | 936 | mG*mC*mC*mA*mG*G*C*T*G*G* | 1688 | XXXXXXXXX | 20mers, Full PS, 5-10- | Malat1 |
| 3182 | GTTATGACUCA | | T*T*A*T*G*mA*mC*mU*mC*mA | | XXXXXXXXXX | 5 2′OMe gapmers | |
| WV- | AAAGGCTTA | 937 | mA*mA*mA*mG*mG*C*T*T*A*G* | 1689 | XXXXXXXXX | 20mers, Full PS, 5-10- | Malat1 |
| 3183 | GCGCCCACCUC | | C*G*C*C*C*mA*mC*mC*mU*mC | | XXXXXXXXXX | 5 2′OMe gapmers | |
| WV- | GGAUUGGGA | 938 | mG*mG*mA*mU*mU*G*G*G*A*G* | 1690 | XXXXXXXXX | 20mers, Full PS, 5-10- | Malat1 |
| 3184 | GTTACTUGCCA | | T*T*A*C*T*mU*mG*mC*mC*mA | | XXXXXXXXXX | 5 2′OMe gapmers | |
| WV- | GUCCUGGAA | 939 | mG*mU*mC*mC*mU*G*G*A*A*A* | 1691 | XXXXXXXXX | 20mers, Full PS, 5-10- | Malat1 |
| 3185 | ACCAGGAGU | | C*C*A*G*G*mA*mG*mU*mG*mC | | XXXXXXXXXX | 5 2′OMe gapmers | |
| | GC | | | | | | |
| WV- | CAGGCTGGTT | 940 | mC*mA*mG*mG*mC*T*G*G*T*T* | 1692 | XXXXXXXXX | 20mers, Full PS, 5-10- | Malat1 |
| 3186 | ATGACUCAGA | | A*T*G*A*C*mU*mC*mA*mG*mA | | XXXXXXXXXX | 5 2′OMe gapmers | |
| WV- | GGGAGTTACT | 941 | mG*mG*mG*mA*mG*T*T*A*C*T*T | 1693 | XXXXXXXXX | 20mers, Full PS, 5-10- | Malat1 |
| 3187 | TGCCAACUUG | | *G*C*C*A*mA*mC*mU*mU*mG | | XXXXXXXXXX | 5 2′OMe gapmers | |
| WV- | UGGGAGTTA | 942 | mU*mG*mG*mG*mA*G*T*T*A*C* | 1694 | XXXXXXXXX | 20mers, Full PS, 5-10- | Malat1 |
| 3188 | CTTGCCAACUU | | T*T*G*C*C*mA*mA*mC*mU*mU | | XXXXXXXXXX | 5 2′OMe gapmers | |
| WV- | UUGGGAGTT | 943 | mU*mU*mG*mG*mG*A*G*T*T*A* | 1695 | XXXXXXXXX | 20mers, Full PS, 5-10- | Malat1 |
| 3189 | ACTTGCCAACU | | C*T*T*G*C*mC*mA*mA*mC*mU | | XXXXXXXXXX | 5 2′OMe gapmers | |
| WV- | AUUUCCTCA | 944 | mA*mU*mU*mU*mC*C*T*C*A*A* | 1696 | XXXXXXXXX | 20mers, Full PS, 5-10- | Malat1 |
| 3190 | ACACTCAGCCU | | C*A*C*T*C*mA*mG*mC*mC*mU | | XXXXXXXXXX | 5 2′OMe gapmers | |
| WV- | CCCCUCCCTC | 945 | mC*mC*mC*mC*mU*C*C*C*T*C*A | 1697 | XXXXXXXXX | 20mers, Full PS, 5-10- | Malat1 |
| 3191 | ATCAACAAAA | | *T*C*A*A*mC*mA*mA*mA*mA | | XXXXXXXXXX | 5 2′OMe gapmers | |
| WV- | ACAUUTCCAC | 946 | mA*mC*mA*mU*mU*T*C*C*A*C* | 1698 | XXXXXXXXX | 20mers, Full PS, 5-10- | Malat1 |
| 3192 | TTGCCAGUUA | | T*T*G*C*C*mA*mG*mU*mU*mA | | XXXXXXXXXX | 5 2′OMe gapmers | |
| WV- | AAAAGGCTT | 947 | mA*mA*mA*mA*mG*G*C*T*T*A* | 1699 | XXXXXXXXX | 20mers, Full PS, 5-10- | Malat1 |
| 3193 | AGCGCCCACCU | | G*C*G*C*C*mC*mA*mC*mC*mU | | XXXXXXXXXX | 5 2′OMe gapmers | |
| WV- | ACCUGTCTGA | 948 | mA*mC*mC*mU*mG*T*C*T*G*A* | 1700 | XXXXXXXXX | 20mers, Full PS, 5-10- | Malat1 |
| 3194 | GGCAAACGAA | | G*G*C*A*A*mA*mC*mG*mA*mA | | XXXXXXXXXX | 5 2′OMe gapmers | |
| WV- | AUUGGGAGT | 949 | mA*mU*mU*mG*mG*G*A*G*T*T* | 1701 | XXXXXXXXX | 20mers, Full PS, 5-10- | Malat1 |
| 3195 | TACTTGCCAAC | | A*C*T*T*G*mC*mC*mA*mA*mC | | XXXXXXXXXX | 5 2′OMe gapmers | |
| WV- | UCAACAAAA | 950 | mU*mC*mA*mA*mC*A*A*A*A*G* | 1702 | XXXXXXXXX | 20mers, Full PS, 5-10- | Malat1 |
| 3196 | GCCCACCCUCU | | C*C*C*A*C*mC*mC*mU*mC*mU | | XXXXXXXXXX | 5 2′OMe gapmers | |
| WV- | CUAAGATGCT | 951 | mC*mU*mA*mA*mG*A*T*G*C*T* | 1703 | XXXXXXXXX | 20mers, Full PS, 5-10- | Malat1 |
| 3197 | AGCTTGGCCA | | A*G*C*T*T*mG*mG*mC*mC*mA | | XXXXXXXXXX | 5 2′OMe gapmers | |
| WV- | GGGTCAGCTG | 952 | L001Geo*Geo*Geo*Teo*m5Ceo*A*G | 1704 | OXXXXXXXX | C6 amine PO linker, | Malat1 |
| 3356 | CCAATGCTAG | | *C*T*G*C*C*A*A*T*Geo*m5Ceo*T | | XXXXXXXXX | MOE, full-PS | |
| | | | eo*Aeo*Geo | | XX | | |
| WV- | GGGTCAGCTG | 953 | Mod030Geo*Geo*Geo*Teo*m5Ceo*A | 1705 | OXXXXXXXX | WV-2735 based; with | Malat1 |
| 3521 | CCAATGCTAG | | *G*C*T*G*C*C*A*A*T*Geo*m5Ceo | | XXXXXXXXX | PO linker, Laurie acid | |
| | | | *Teo*Aeo*Geo | | XX | | |
| WV- | GGGTCAGCTG | 954 | Mod031Geo*Geo*Geo*Teo*m5Ceo*A | 1706 | OXXXXXXXX | WV-2735 based; with | Malat1 |
| 3522 | CCAATGCTAG | | *G*C*T*G*C*C*A*A*T*Geo*m5Ceo | | XXXXXXXXX | PO inker, Myristic | |
| | | | *Teo*Aeo*Geo | | XX | acid | |
| WV- | GGGTCAGCTG | 955 | Mod032Geo*Geo*Geo*Teo*m5Ceo*A | 1707 | OXXXXXXXX | WV-2735 based; with | Malat1 |
| 3523 | CCAATGCTAG | | *G*C*T*G*C*C*A*A*T*Geo*m5Ceo | | XXXXXXXXX | PO linker, Palmitic | |
| | | | *Teo*Aeo*Geo | | XX | acid | |
| WV- | GGGTCAGCTG | 956 | Mod033Geo*Geo*Geo*Teo*m5Ceo*A | 1708 | OXXXXXXXX | WV-2735 based; with | Malat1 |
| 3524 | CCAATGCTAG | | *G*C*T*G*C*C*A*A*T*Geo*m5Ceo | | XXXXXXXXX | PO linker, Stearic acid | |
| | | | *Teo*Aeo*Geo | | XX | | |

¹Including —C(O)— (noted as O) connecting Mod and the amino group of C6 amino linker and phosphate or phosphorothioate linkage connecting C6 amino linker and oligonucleotide chain (noted as X (stereorandom), S (Sp) or R (Sp)).

### Abbreviations:

2\′: 2′

### 5Ceo: 5-Methyl 2′-Methoxyethyl C

C6: C6 amino linker (L001, —NH—(CH₂)₆— wherein —NH— is connected to Mod (through —C(O)—) or —H, and —(CH₂)₆— is connected to the 5′-end of oligonucleotide chain through, e.g., phosphodiester (illustrated in the Table as O or PO), phosphorothioate (illustrated in the Table as * if the phosphorothioate not chirally controlled; *S, S, or Sp, if chirally controlled and has an Sp configuration, and *R, R, or Rp, if chirally controlled and has an Rp configuration), or phosphorodithioate (illustrated in the Table as PS2 or :). May also be referred to as C6 linker or C6 amine linker)

### eo: 2′-MOE

### Exon: Exon of Dystrophin

### F, f: 2′-F

Lauric (in Mod013), Myristic (in Mod014), Palmitic (in Mod005), Stearic (in Mod015), Oleic (in Mod016), Linoleic (in Mod017), alpha-Linoleinc (in Mod018), gamma-Linolenic (in Mod019), DHA (in Mod006), Turbinaric (in Mod020), Dilinoleic (in Mod021), TriGlcNAc (in Mod024), TrialphaMannose (in Mod026), MonoSulfonamide (in Mod 027), TriSulfonamide (in Mod029), Lauric (in Mod030), Myristic (in Mod031), Palmitic (in Mod032), and Stearic (in Mod033): Lauric acid (for Mod013), Myristic acid (for Mod014), Palmitic acid (for Mod005), Stearic acid (for Mod015), Oleic acid (for Mod016), Linoleic acid (for Mod017), alpha-Linolenic acid (for Mod018), gamma-Linolenic acid (for Mod019), docosahexaenoic acid (for Mod006), Turbinaric acid (for Mod020), alcohol for Dilinoleyl (for Mod021), acid for TriGlcNAc (for Mod024), acid for TrialphaMannose (for Mod026), acid for MonoSulfonamide (for Mod 027), acid for TriSulfonamide (for Mod029), Lauryl alcohol (for Mod030), Myristyl alcohol (for Mod031), Palmityl alcohol (for Mod032), and Stearyl alcohol (for Mod033), respectively, conjugated to oligonucleotide chains through amide groups, C6 amino linker, phosphodiester linkage (PO), and/or phosphorothioate linkage (PS): Mod013 (Lauric acid with C6 amino linker and PO or PS), Mod014 (Myristic acid with C6 amino linker and PO or PS), Mod005 (Palmitic acid with C6 amino linker and PO or PS), Mod015 (Stearic acid with C6 amino linker and PO or PS), Mod016 (Oleic acid with C6 amino linker and PO or PS), Mod017 (Linoleic acid with C6 amino linker and PO or PS), Mod018 (alpha-Linolenic acid with C6 amino linker and PO or PS), Mod019 (gamma-Linolenic acid with C6 amino linker and PO or PS), Mod006 (DHA with C6 amino linker and PO or PS), Mod020 (Turbinaric acid with C6 amino linker and PO or PS), Mod021 (alcohol (see below) with PO or PS), Mod024 (acid (see below) with C6 amino linker and PO or PS), Mod026 (acid (see below) with C6 amino linker and PO or PS), Mod027 (acid (see below) with C6 amino linker and PO or PS), Mod029 (acid (see below) with C6 amino linker and PO or PS), Mod030 (Lauryl alcohol with PO or PS), Mod031 (Myristyl alcohol with PO or PS), Mod032 (Palmityl alcohol with PO or PS), and Mod033 (Stearyl alcohol with PO or PS), with PO or PS for each oligonucleotide indicated in the Table (for example, WV-3473 Lauric acid conjugated to oligonucleotide chain of WV-3473 via amide group, C6, and PO:
Mod013L001fU*SfC*SfA*SfA*SfG*SfG*SmAfA*SmGmA*SfU*SmGmGfC*SfA*SfU*SfU*SfU*SfC*SfU (SEQ ID NO: 1709) (Description), OOSSSSSSOSOSSOOSSSSSS (Stereochemistry), and/or WV-3473, Lauric acid, C6 PO linker (Notes); WV-3557 Steary alcohol conjugated to oligonucleotide chain of WV-3473 via PS:
Mod033*fU*SfC*SfA*SfA*SfG*SfG*SmAfA*SmGmA*SfU*SmGmGfC*SfA*SfU*SfU*SfU*SfC*SfU (SEQ ID NO: 1710) (Description), XSSSSSSOSOSSOOSSSSSS (Stereochemistry), and/or WV-3473, Stearic PS (Notes); and WV-4106 Stearic acid conjugated to oligonucleotide chain of WV-3473 via amide group, C₆, and PS:
Mod015L001*fU*SfC*SfA*SfA*SfG*SfG*SmAfA*SmGmA*SfU*SmGmGfC*SfA*SfU*SfU*SfU*SfC*SfU (SEQ ID NO: 1711) (Description), OXSSSSSSOSOSSOOSSSSSS (Stereochemistry), and/or WV-3473, C6 PS linker, Stearic acid (Notes)) Moieties for conjugation, and example reagents (many of which were previously known and are commercially available or can be readily prepared using known technologies in accordance with the present disclosure, e.g., Lauric acid (for Mod013), Myristic acid (for Mod014), Palmitic acid (for Mod005), Stearic acid (for Mod015), Oleic acid (for Mod016), Linoleic acid (for Mod017), alpha-Linolenic acid (for Mod018), gamma-Linolenic acid (for Mod019), docosahexaenoic acid (for Mod006), Turbinaric acid (for Mod020), alcohol for Dilinoleyl (for Mod021), Lauryl alcohol (for Mod030), Myristyl alcohol (for Mod031), Palmityl alcohol (for Mod032), Stearyl alcohol (for Mod033), etc.) are listed below

### m: 2′-OMe.

NA: Not Applicable; this term is generally used for negative controls

### OMe: 2′-OMe

O, PO: phoshodiester (phosphate), or when used with Mod and L001, —C(O)— (connecting Mod and L001, for example, Mod013L001fU*SfC*SfA*SfA*SfG*SfG*SmAfA*SmGmA*SfU*SmGmGfC*SfA*SfU*SfU*SfU*SfC*SfU (SEQ ID NO: 1709) (Description), OOSSSSSSOSOSSOOSSSSSS (Stereochemistry) and/or WV-3473, Lauric acid, C₆ PO linker (Notes). Note the second O OOSSSSSSOSOSSOOSSSSSS (Stereochemistry) represents phosphodiester linkage connecting L001 and 5′-O— of oligonucleotide chain: Mod013L001fU*SfC*SfA*SfA*SfG*SfG*SmAfA*SmGmA*SfU*SmGmGfC*SfA*SfU*SfU*SfU*SfC*SfU (SEQ ID NO: 1709))

### *, PS: Phosphorothioate

PS2, ::phosphorodithioate (e.g., WV-3078, wherein a colon (:) indicates a phosphorodithioate)
*R, R, Rp: Phosphorothioate in Rp conformation
*S, S, Sp: Phosphorothioate in Sp conformation

### WV, W V-: WV-

X: Phosphorothioate stereorandom
Example moieties (e.g., lipid moieties, targeting component, etc.) and example preparation reagents (e.g., acids, alcohols, etc.) for conjugation to prepare provided oligonucleotides, e.g., example oligonucleotides in Tables 1-4 comprising such moieties, in accordance with the present disclosure include the below:

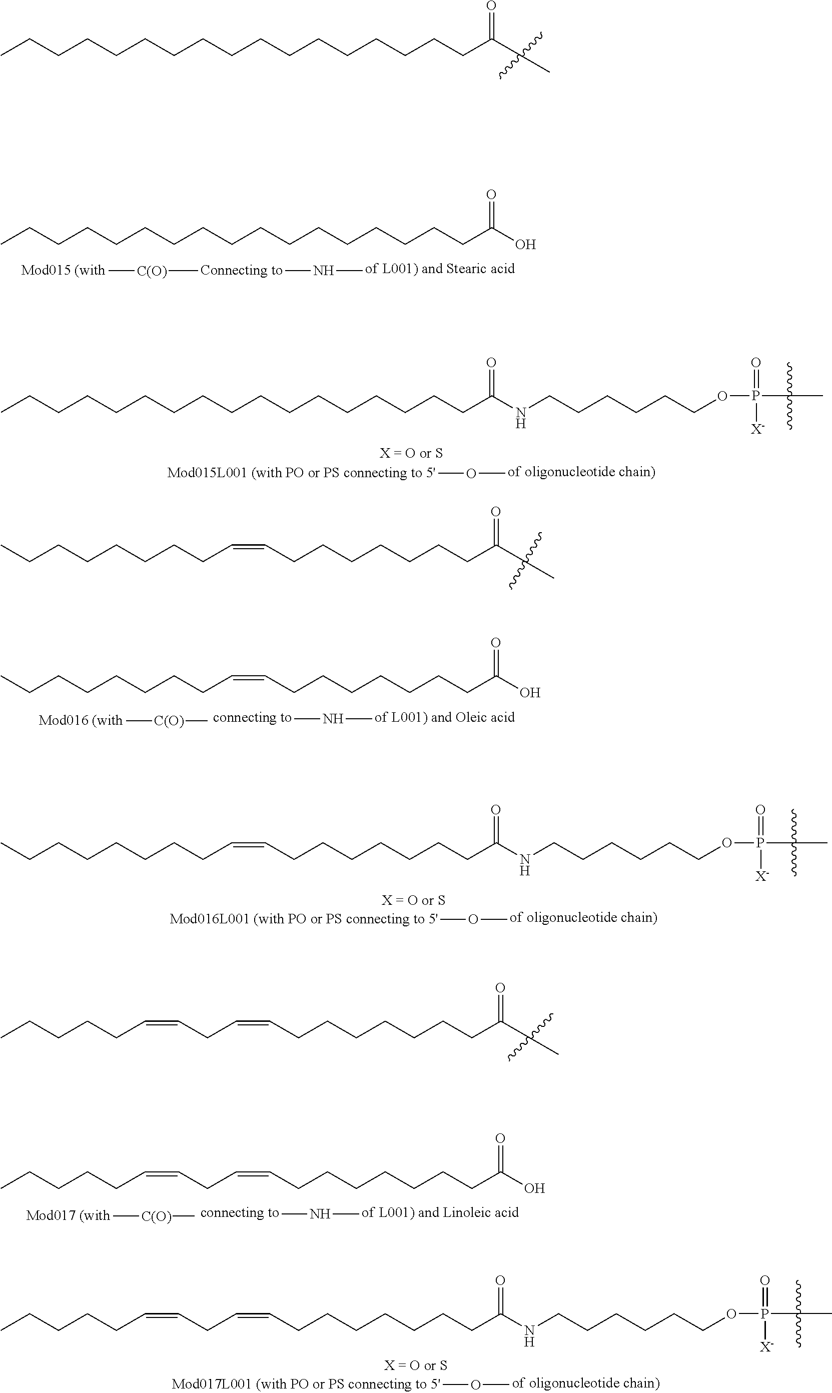

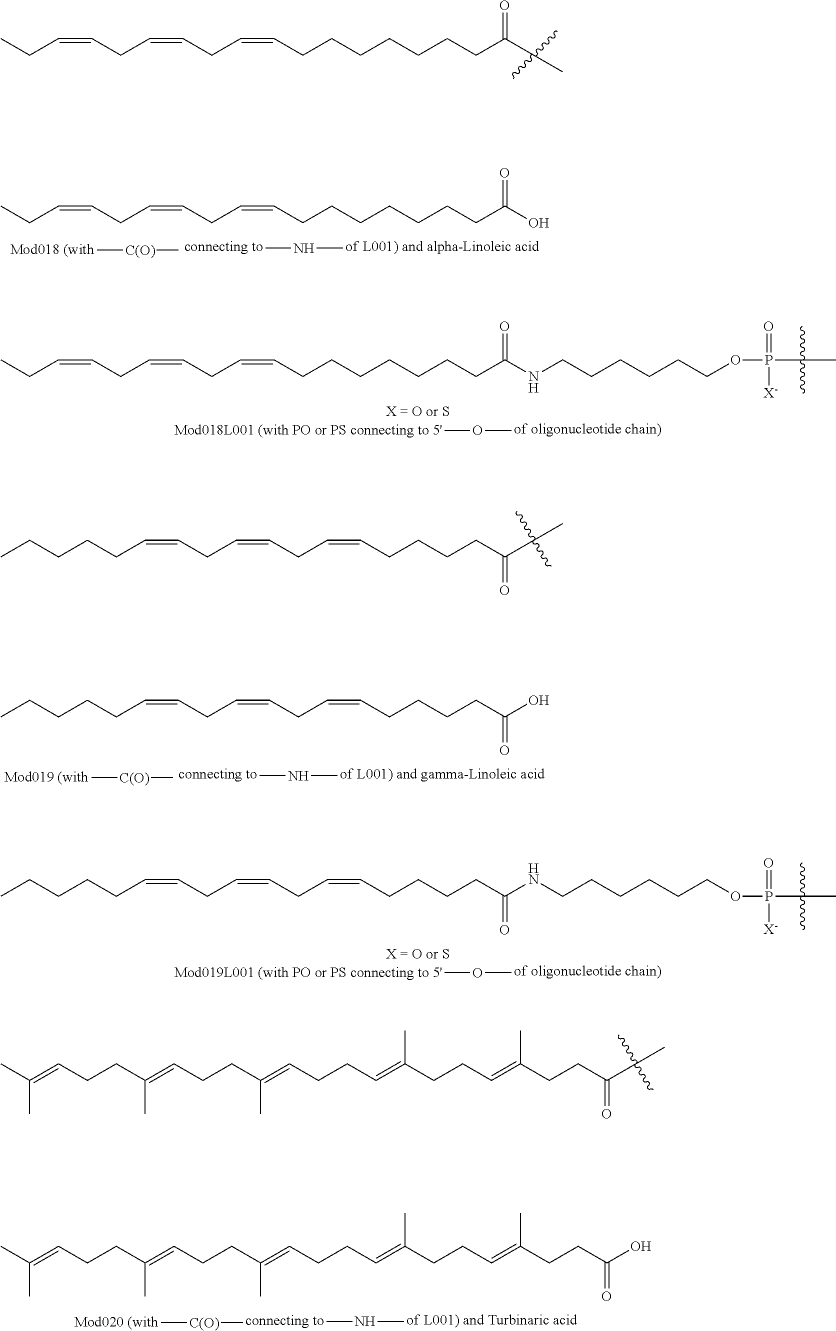

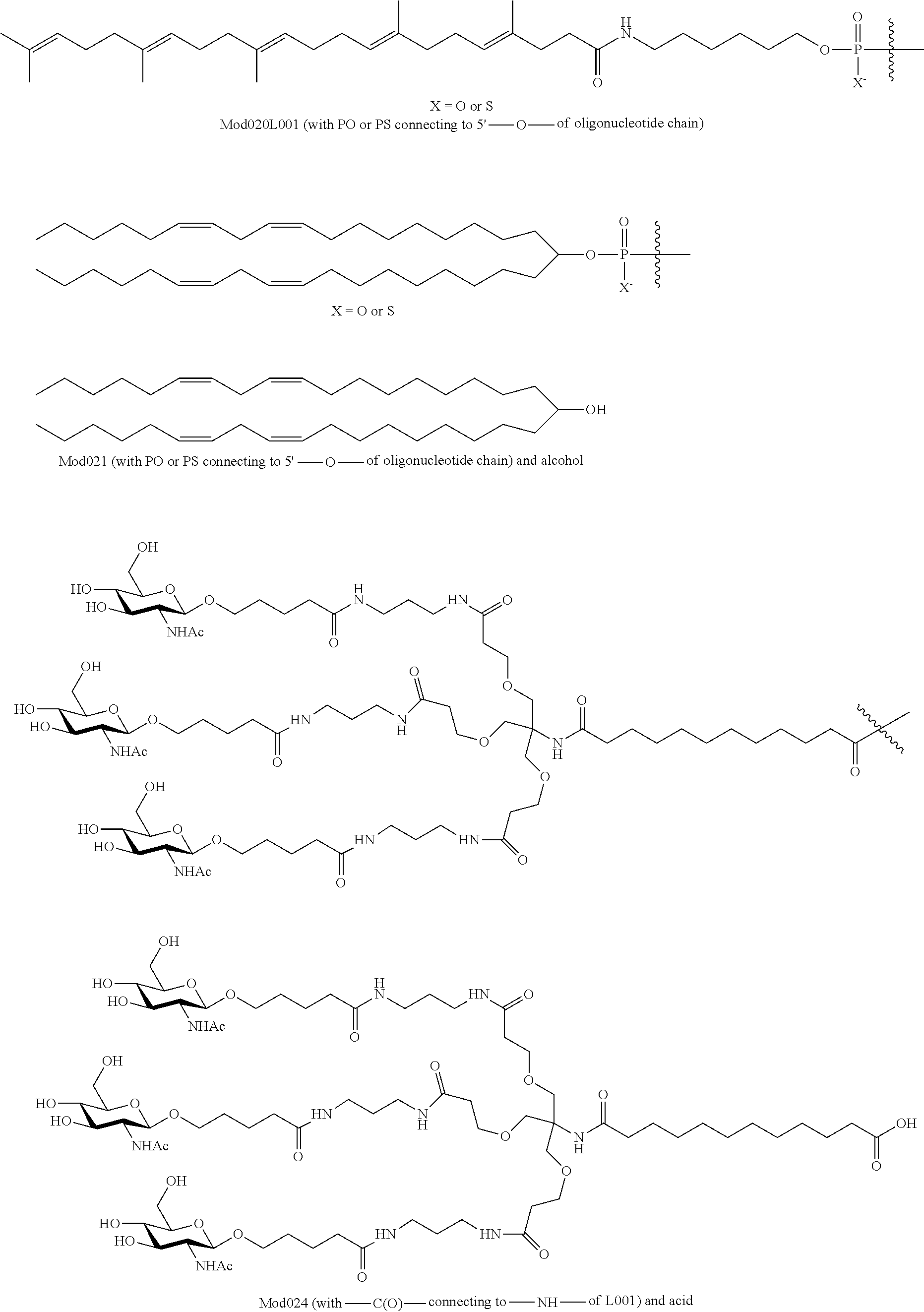

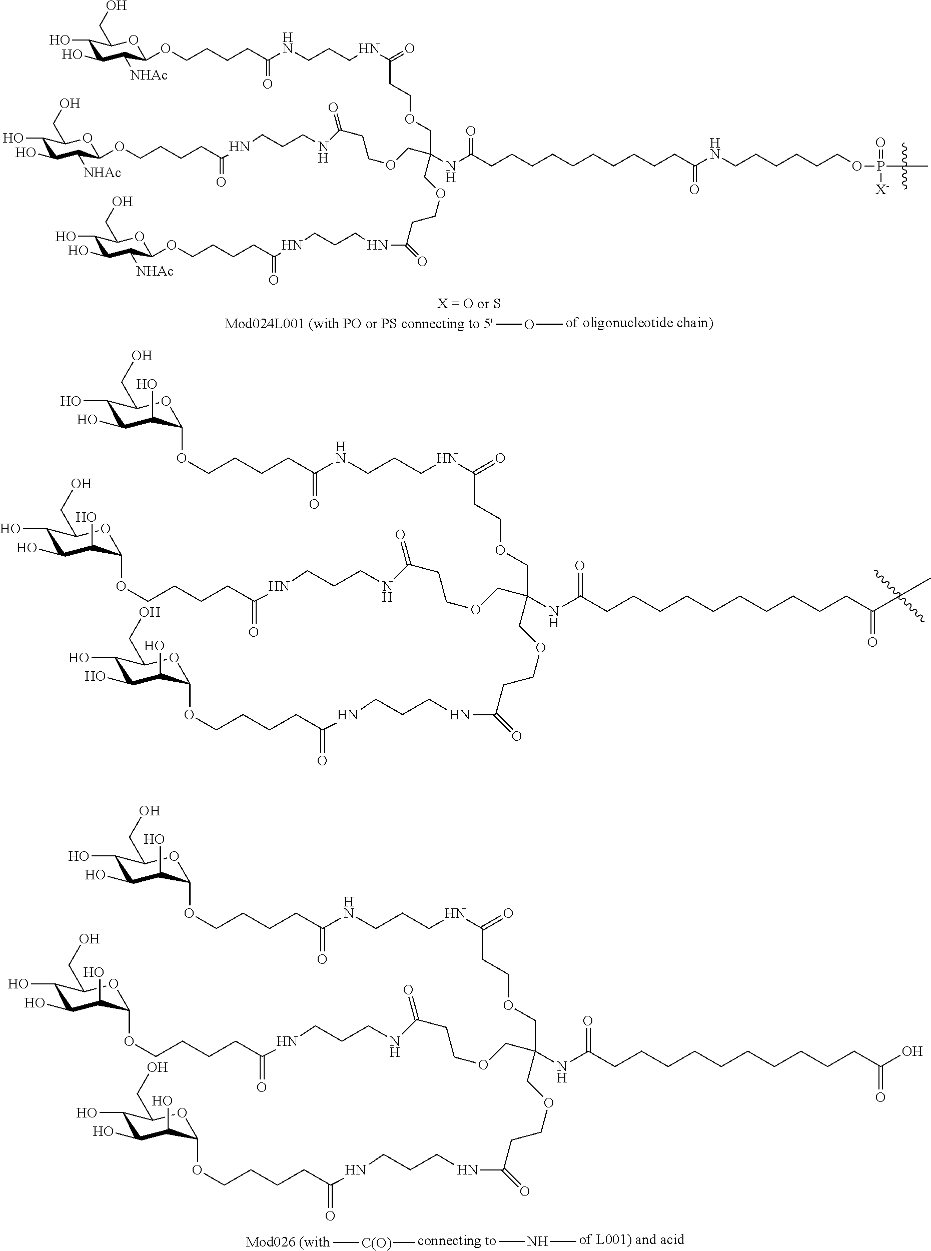

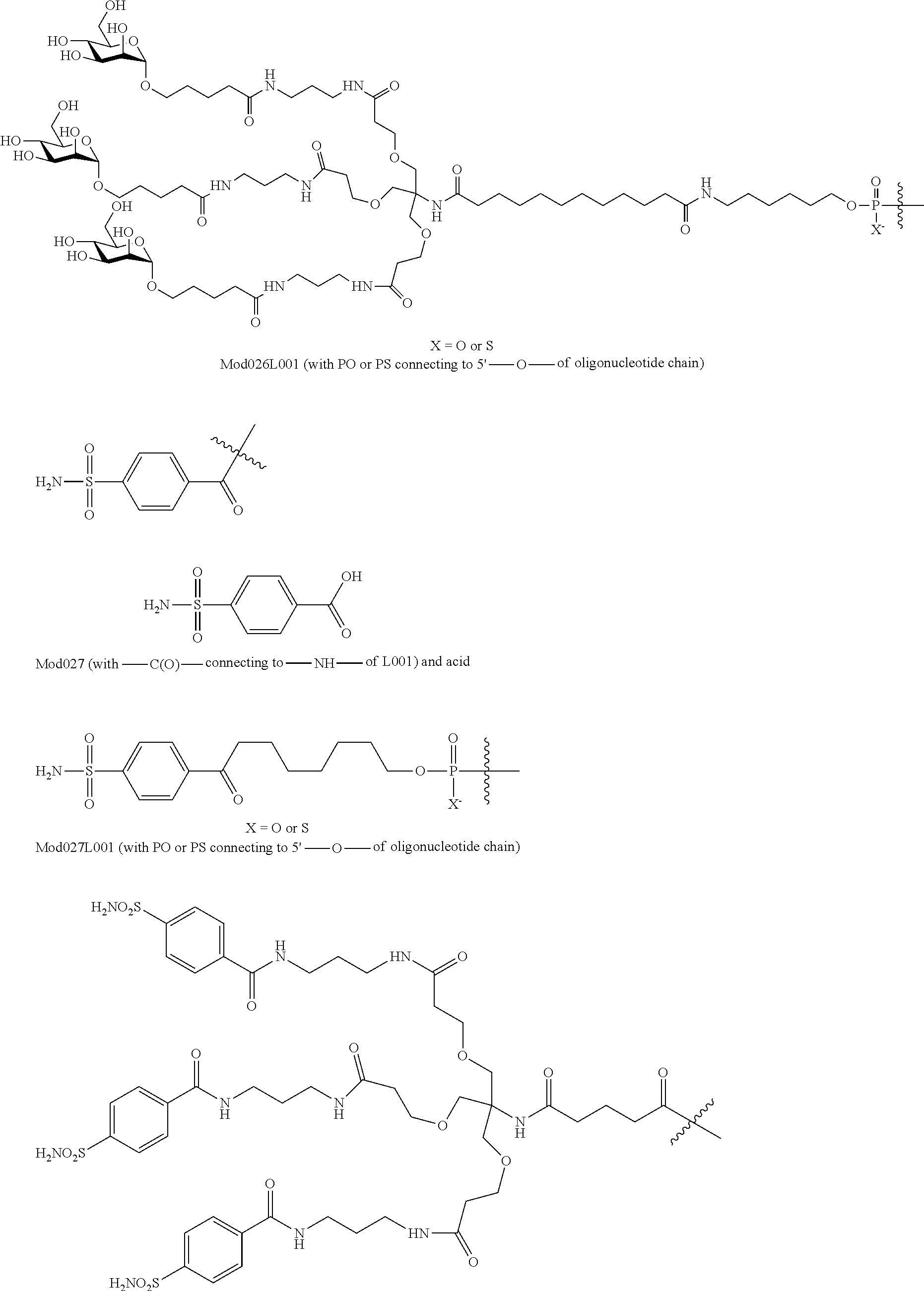

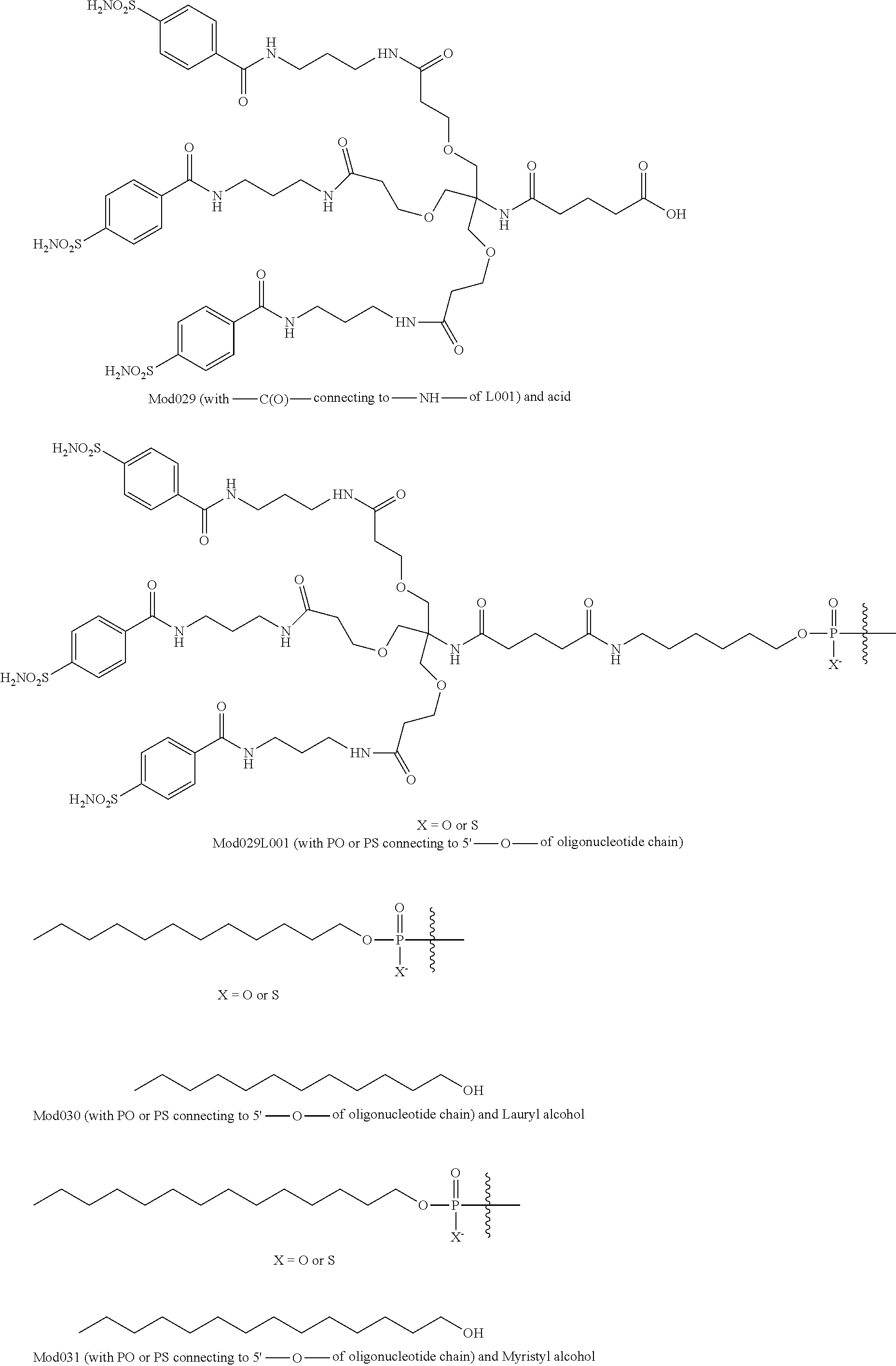

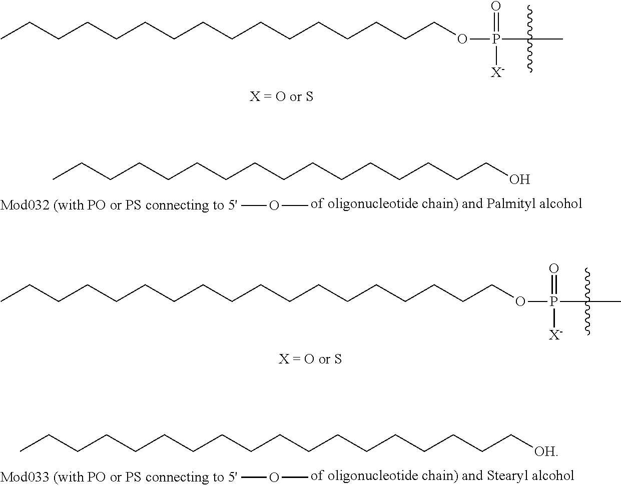

Applicant notes that presented above in the Table are example ways of presenting structures of provided oligonucleotides, for example, WV-3546 (Mod020L001fU*SfC*SfA*SfA*SfG*SfG*SmAfA*SmGmA*SfU*SmGmGfC*SfA*SfU*SfU*SfU*SfC*SfU (SEQ ID NO: 1712)) can be presented as a lipid moiety

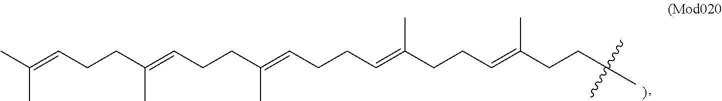 [SMILES: *CC/C(C)=C/CC/C(C)=C/CC/C=C(\C)CC/C=C(\C)CCC=C(C)C]

connected via —C(O)— (QOSSSSSSOSOSSOOSSSSSS) to the —NH— of —NH—(CH₂)₆—, wherein the —(CH₂)₆— is connected to the 5′-end of the oligonucleotide chain via a phosphodiester linkage (OOSSSSSSOSOSSOOSSSSSS). One having ordinary skill in the art understands that a provided oligonucleotide can be presented as combinations of lipid, linker and oligonucleotide chain units in many different ways, wherein in each way the combination of the units provides the same oligonucleotide. For example, WV-3546, can be considered to have a structure of A^{c}-[-L^{LD}-(R^{LD})ₐ]_{b}, wherein a is 1, b is 1, and have a lipid moiety R^{LD} of

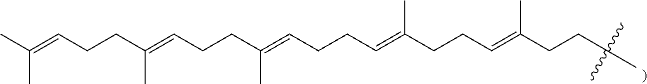 [SMILES: *CC/C(C)=C/CC/C(C)=C/CC/C=C(\C)CC/C=C(\C)CCC=C(C)C]

connected to its oligonucleotide chain (A^{c}) portion through a linker L^{LD} of —C(O)—NH—(CH₂)₆—OP(═O)(OH)—O—, wherein —C(O)— is connected to R^{LD} and —O— is connected to A^{c} (as 5′-O— of the oligonucleotide chain); one of the many alternative ways is that R^{LD} is

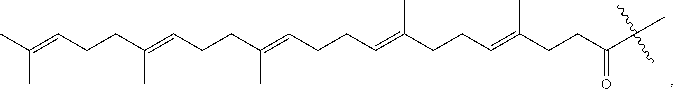 [SMILES: *C(=O)CC/C(C)=C/CC/C(C)=C/CC/C=C(\C)CC/C=C(\C)CCC=C(C)C]

and L^{LD} is —NH—(CH₂)₆—OP(═O)(OH)—O—, wherein —NH— is connected to R^{LD}, and —O— is connected to A^{c} (as 5′-O— of the oligonucleotide chain).

Oligonucleotides were prepared and characterized using a variety of methods in accordance of the present disclosure. Example MS data are presented below:

| WAVE | Calculated | Found |
|---|---|---|
| ID | Mass | Mass |
| WV-2531 | 6767.90000 | 6766.3 |
| WV-3152 | 6743.77000 | 6742.8 |
| WV-3472 | 6720.78472 | 6720.8 |
| WV-3473 | 6732.82024 | 6735 |
| WV-3507 | 6716.75464 | 6717.3 |
| WV-3508 | 6704.71912 | 6706 |
| WV-3509 | 6716.75464 | 6718 |
| WV-3510 | 6716.75464 | 6717.6 |
| WV-3511 | 6728.79016 | 6731 |
| WV-3512 | 6700.68904 | 6702 |
| WV-3513 | 6712.72456 | 6713 |
| WV-3514 | 6688.65352 | 6688.9 |
| WV-3515 | 6700.68904 | 6701.2 |
| WV-3545 | 7178.43622 | 7178 |
| WV-3546 | 7294.59604 | 7295 |

*Calculated and found mass data of WV-2531 and WV-3152 are for sodium adducts.

In various embodiments, a composition comprises a lipid and a nucleic acid [as non-limiting examples: an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, or a vector, or a portion thereof] which targets any gene listed herein.

In some embodiments, a composition comprises a lipid and a nucleic acid which targets any of: AFF2, APOB, APOC3, AR, ATM, ATN1, ATXN1, ATXN10, ATXN2, ATXN3, ATXN7, ATXN80S, BACE1, BBS1, BCL2L1, BRCA1, BRCA2, C9orf72, CACNA1A, CD40, CD40, CDKN1A, CFTR, CLC1, CNBP, COL7A1, CYPI 1A, DMD, DMPK, DYSF, Dystrophin, ERBB2, F7, F9, FANCC, FGB, FGFR1, FKTN, FLT1, FMR1, FXN, GHR, GRP143, HBB, HNRNPH1, HTT (Huntingtin), IKBKAP, IL5RA, ISCU, JPH3, KDR, LMNA, MAPT, MCL1, MDM2, MLC1, MST1R, MSTN, MUT, MYC, NF1, NPC1, PCCA, PCCB, PHB, PKM, PMM2, PPP2R2B, PTCH1, PTS, PTS, RHO, RHO, RPGR, RPGR, SMN2, SRA1, STAT3, TBP, TERT, TMPRSS2, TNFRSFIB, USH1C, USP5, and WT1.

In some embodiments, the common base sequence is capable of hybridizing with a transcript in a cell. In some embodiments, a common base sequence hybridizes with a transcript of any gene described herein or known in the art.

In some embodiments, a composition comprises a lipid and a biologically active agent suitable for treatment of any of: Afibrinogenemia, Alzheimer's disease, Alzheimer's disease/FTDP-17 Taupathies, Ataxia telangiectasia, Bardet-Biedl syndrome, Beta-thalassemia, Cancer, CDG1A, Congenital adrenal insufficiency, Cystic fibrosis, Dentatorubral-pallidoluysian atrophy, Duchenne muscular dystrophy, Dystrophic epidermolysis bullosa, Factor VII deficiency, Familial dysautonomia, Fanconi anemia, FHBL/atherosclerosis, Fragile X mental retardation, Fragile X syndrome, Friedreich's ataxia, Frontotemporal dementia, Fukuyama congenital muscular dystrophy (FCMD), Growth hormone insensitivity, Hemophilia A, HPABH4A, Huntington's Diease, Huntington's Disease-like 2, Hutchinson-Gilford progeria (HGPS), Immune-response, Inflammatory disease, Influenza virus, Machado-Joseph disease, Mental retardation, Mental retardation, X-linked, associated with FRAXE, Methylmalonic aciduria, Miyoshi myopathy, MLC1, Muscle wasting diseases, Myopathy with lactic acidosis, Myotonic dystrophy, Neurofibromatosis, Niemann-Pick type C, Ocular albinism type 1, Oculpopharyngeal muscular dystrophy, Propionic acidemia, Retinitis pigmentosa, Spinal muscular atrophy, Spinocerebellar ataxia, Spinocerebellar ataxia type 1, Spinomuscular bulbar atrophy, or Usher syndrome.

In some embodiments, an antisense oligonucleotide is an oligonucleotide which participates in RNaseH-mediated cleavage; for example, an antisense oligonucleotide hybridizes in a sequence-specific manner to a portion of a target mRNA, thus targeting the mRNA for cleavage my RNaseH. In some embodiments, an antisense oligonucleotide is able to differentiate between a wild-type and a mutant allele of a target. In some embodiments, an antisense oligonucleotide significantly participates in RNaseH-mediated cleavage of a mutant allele but participates in RNaseH-mediated cleavage of a wild-type allele to a much less degree (e.g., does not significantly participate in RNaseH-mediated cleavage of the wild-type allele of the target).

In various embodiments, a composition comprises a lipid and a nucleic acid [as non-limiting examples: an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, or a vector, or a portion thereof] which targets Huntingtin gene.

In various embodiments, a composition comprises a lipid and a nucleic acid [as non-limiting examples: an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, or a vector, or a portion thereof] which targets a mutant allele of Huntingtin gene.

In various embodiments, a composition comprises a lipid and a nucleic acid [as non-limiting examples: an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, lncRNA, ncRNA or other nucleic acid), a plasmid, or a vector, or a portion thereof] which is capable of differentiating between a wild-type and a mutant allele of Huntingtin gene.

Various oligonucleotides to HTT (Huntingtin gene) are listed below, in Table 8.

TABLE 8
Example HTT Oligonucleotides.

| | | SEQ | | SEQ | | | |
|---|---|---|---|---|---|---|---|
| | | ID | | ID | | | |
| WAVE ID | Base Sequence | NO: | Description | NO: | Stereochemistry | Notes 1 | Notes 2 |
| ONT-450 | ATTAATAAATT | 1713 | A*T*T*A*A*T*A*A*A*T* | 2065 | XXXXXXXXX | Stereorandom | Htt SNP |
| | GTCATCACC | | T*G*T*C*A*T*C*A*C*C | | XXXXXXXXXX | Htt sequence | rs7685686 |
| ONT-451 | ATTAATAAATT | 1714 | A*ST*ST*SA*SA*ST*SA*S | 2066 | SSSSSSSSSSSS | Stereopure Htt | Htt SNP |
| (WV-451) | GTCATCACC | | A*SA*ST*ST*SG*ST*SC*R | | SRSSSSS | sequence I | rs7685686 |
| | | | A*ST*SC*SA*SC*SC | | | | |
| ONT-452 | ATTAATAAATT | 1715 | A*ST*ST*SA*SA*ST*SA*S | 2067 | SSSSSSSSSSSS | Stereopure Htt | Htt SNP |
| | GTCATCACC | | A*SA*ST*ST*SG*ST*SC*S | | SSRSSSS | sequence II | rs7685686 |
| | | | A*RT*SC*SA*SC*SC | | | | |
| ONT-453 | GGUGAUGACA | 1716 | rGrGrUrGrArUrGrArCrArAr | 2068 | OOOOOOOOO | RNA against Htt | Htt SNP |
| | AUUUAUUAAU | | UrUrUrArUrUrArArU | | OOOOOOOOOO | sequence Mutant | rs7685686 |
| ONT-454 | GGUGAUGGCA | 1717 | rGrGrUrGrArUrGrGrCrArAr | 2069 | OOOOOOOOO | RNA against Htt | Htt SNP |
| | AUUUAUUAAU | | UrUrUrArUrUrArArU | | OOOOOOOOOO | sequence Wild | rs7685686 |
| | | | | | | Type | |
| WV-902 | UUUGGAAGUC | 1718 | rUrUrUrGrGrArArGrUrCrUr | 2070 | OOOOOOOOO | wtRNA | muHTT SNP |
| | UGCGCCCUUGU | | GrCrGrCrCrCrUrUrGrUrGrC | | OOOOOOOOO | | 362307 |
| | GCCC | | rCrC | | OOOOOO | | |
| WV-903 | UUUGGAAGUC | 1719 | rUrUrUrGrGrArArGrUrCrUr | 2071 | OOOOOOOOO | mRNA | muHTT SNP |
| | UGUGCCCUUGU | | GrUrGrCrCrCrUrUrGrUrGrC | | OOOOOOOOO | | 362307 |
| | GCCC | | rCrC | | OOOOOO | | |
| WV-904 | GGGCACAAGG | 1720 | G*G*G*C*A*C*A*A*G*G* | 2072 | XXXXXXXXX | ASO1 All DNA; | muHTT SNP |
| | GCACAGACTT | | G*C*A*C*A*G*A*C*T*T | | XXXXXXXXXX | stereorandom PS | 362307 |
| WV-905 | GGCACAAGGGC | 1721 | G*G*C*A*C*A*A*G*G*G* | 2073 | XXXXXXXXX | ASO2 All DNA; | muHTT SNP |
| | ACAGACTTC | | C*A*C*A*G*A*C*T*T*C | | XXXXXXXXXX | stereorandom PS | 362307 |
| WV-906 | GCACAAGGGCA | 1722 | G*C*A*C*A*A*G*G*G*C* | 2074 | XXXXXXXXX | ASO3 All DNA; | muHTT SNP |
| | CAGACTTCC | | A*C*A*G*A*C*T*T*C*C | | XXXXXXXXXX | stereorandom PS | 362307 |
| WV-907 | CACAAGGGCAC | 1723 | C*A*C*A*A*G*G*G*C*A* | 2075 | XXXXXXXXX | ASO4 All DNA; | muHTT SNP |
| | AGACTTCCA | | C*A*G*A*C*T*T*C*C*A | | XXXXXXXXXX | stereorandom PS | 362307 |
| WV-908 | ACAAGGGCACA | 1724 | A*C*A*A*G*G*G*C*A*C* | 2076 | XXXXXXXXX | ASO5 All DNA; | muHTT SNP |
| | GACTTCCAA | | A*G*A*C*T*T*C*C*A*A | | XXXXXXXXXX | stereorandom PS | 362307 |
| WV-909 | CAAGGGCACAG | 1725 | C*A*A*G*G*G*C*A*C*A* | 2077 | XXXXXXXXX | ASO6 All DNA; | muHTT SNP |
| | ACTTCCAAA | | G*A*C*T*T*C*C*A*A*A | | XXXXXXXXXX | stereorandom PS | 362307 |
| WV-910 | GGGCACAAGG | 1726 | mG*mG*mG*mC*mA*C*A | 2078 | XXXXXXXXX | ASO7 5-15 (2′- | muHTT SNP |
| | GCACAGACTT | | *A*G*G*G*C*A*C*A*G*A | | XXXXXXXXXX | OMe-DNA); | 362307 |
| | | | *C*T*T | | | stereorandom PS | |
| WV-911 | GGCACAAGGGC | 1727 | mG*mG*mC*mA*mC*A*A | 2079 | XXXXXXXXX | ASO8 5-15 (2′- | muHTT SNP |
| | ACAGACTTC | | *G*G*G*C*A*C*A*G*A*C | | XXXXXXXXXX | OMe-DNA); | 362307 |
| | | | *T*T*C | | | stereorandom PS | |
| WV-912 | GCACAAGGGCA | 1728 | mG*mC*mA*mC*mA*A*G | 2080 | XXXXXXXXX | ASO9 5-15 (2′- | muHTT SNP |
| | CAGACTTCC | | *G*G*C*A*C*A*G*A*C*T | | XXXXXXXXXX | OMe-DNA); | 362307 |
| | | | *T*C*C | | | stereorandom PS | |
| WV-913 | CACAAGGGCAC | 1729 | mC*mA*mC*mA*mA*G*G | 2081 | XXXXXXXXX | ASO10 5-15 (2′- | muHTT SNP |
| | AGACTTCCA | | *G*C*A*C*A*G*A*C*T*T | | XXXXXXXXXX | OMe-DNA); | 362307 |
| | | | *C*C*A | | | stereorandom PS | |
| WV-914 | ACAAGGGCACA | 1730 | mA*mC*mA*mA*mG*G*G | 2082 | XXXXXXXXX | ASO11 5-15 (2′- | muHTT SNP |
| | GACTTCCAA | | *C*A*C*A*G*A*C*T*T*C* | | XXXXXXXXXX | OMe-DNA); | 362307 |
| | | | C*A*A | | | stereorandom PS | |
| WV-915 | CAAGGGCACAG | 1731 | mC*mA*mA*mG*mG*G*C | 2083 | XXXXXXXXX | ASO12 5-15 (2′- | muHTT SNP |
| | ACTTCCAAA | | *A*C*A*G*A*C*T*T*C*C* | | XXXXXXXXXX | OMe-DNA); | 362307 |
| | | | A*A*A | | | stereorandom PS | |
| WV-916 | GGGCACAAGG | 1732 | mG*mG*mG*mC*mA*C*A | 2084 | XXXXXXXXX | ASO13 5-10-5 | muHTT SNP |
| | GCACAGACUU | | *A*G*G*G*C*A*C*A*mG* | | XXXXXXXXXX | (2′-OMe-DNA- | 362307 |
| | | | mA*mC*mU*mU | | | 2′-OMe); | |
| | | | | | | stereorandom PS | |
| WV-917 | GGCACAAGGGC | 1733 | mG*mG*mC*mA*mC*A*A | 2085 | XXXXXXXXX | ASO14 5-10-5 | muHTT SNP |
| | ACAGACUUC | | *G*G*G*C*A*C*A*G*mA* | | XXXXXXXXXX | (2′-OMe-DNA- | 362307 |
| | | | mC*mU*mU*mC | | | 2′-OMe); | |
| | | | | | | stereorandom PS | |
| WV-918 | GCACAAGGGCA | 1734 | mG*mC*mA*mC*mA*A*G | 2086 | XXXXXXXXX | ASO15 5-10-5 | muHTT SNP |
| | CAGACUUCC | | *G*G*C*A*C*A*G*A*mC* | | XXXXXXXXXX | (2′-OMe-DNA- | 362307 |
| | | | mU*mU*mC*mC | | | 2′-OMe); | |
| | | | | | | stereorandom PS | |
| WV-919 | CACAAGGGCAC | 1735 | mC*mA*mC*mA*mA*G*G | 2087 | XXXXXXXXX | ASO16 5-10-5 | muHTT SNP |
| | AGACUUCCA | | *G*C*A*C*A*G*A*C*mU* | | XXXXXXXXXX | (2′-OMe-DNA- | 362307 |
| | | | mU*mC*mC*mA | | | 2′-OMe); | |
| | | | | | | stereorandom PS | |
| WV-920 | ACAAGGGCACA | 1736 | mA*mC*mA*mA*mG*G*G | 2088 | XXXXXXXXX | ASO17 5-10-5 | muHTT SNP |
| | GACTUCCAA | | *C*A*C*A*G*A*C*T*mU* | | XXXXXXXXXX | (2′-OMe-DNA- | 362307 |
| | | | mC*mC*mA*mA | | | 2′-OMe); | |
| | | | | | | stereorandom PS | |
| WV-921 | CAAGGGCACAG | 1737 | mC*mA*mA*mG*mG*G*C | 2089 | XXXXXXXXX | ASO18 5-10-5 | muHTT SNP |
| | ACTTCCAAA | | *A*C*A*G*A*C*T*T*mC* | | XXXXXXXXXX | (2′-OMe-DNA- | 362307 |
| | | | mC*mA*mA*mA | | | 2′-OMe); | |
| | | | | | | stereorandom PS | |
| WV-922 | GCACAAGGGCA | 1738 | mG*mC*mA*mC*mA*mA* | 2090 | XXXXXXXXX | ASO19 8-7-5 (2′- | muHTT SNP |
| | CAGACUUCC | | mG*mG*G*C*A*C*A*G*A | | XXXXXXXXXX | OMe-DNA-2′- | 362307 |
| | | | *mC*mU*mU*mC*mC | | | OMe); | |
| | | | | | | stereorandom PS | |
| WV-923 | CACAAGGGCAC | 1739 | mC*mA*mC*mA*mA*mG* | 2091 | XXXXXXXXX | ASO20 7-7-6 (2′- | muHTT SNP |
| | AGACUUCCA | | mG*G*C*A*C*A*G*A*mC | | XXXXXXXXXX | OMe-DNA-2′- | 362307 |
| | | | *mU*mU*mC*mC*mA | | | OMe); | |
| | | | | | | stereorandom PS | |
| WV-924 | ACAAGGGCACA | 1740 | mA*mC*mA*mA*mG*mG* | 2092 | XXXXXXXXX | ASO21 6-7-5 (2′- | muHTT SNP |
| | GACUUCCAA | | G*C*A*C*A*G*A*mC*mU | | XXXXXXXXXX | OMe-DNA-2′- | 362307 |
| | | | *mU*mC*mC*mA*mA | | | OMe); | |
| | | | | | | stereorandom PS; | |
| | | | | | | PO in the wings | |
| WV-925 | CAAGGGCACAG | 1741 | mC*mA*mA*mG*mG*G*C | 2093 | XXXXXXXXX | ASO22 5-7-8 (2′- | muHTT SNP |
| | ACUUCCAAA | | *A*C*A*G*A*mC*mU*mU | | XXXXXXXXXX | OMe-DNA-2′- | 362307 |
| | | | *mC*mC*mA*mA*mA | | | OMe); | |
| | | | | | | stereorandom PS; | |
| | | | | | | PO in the wings | |
| WV-926 | GCACAAGGGCA | 1742 | mGmCmAmCmAmAmGmG | 2094 | OOOOOOOXX | ASO23 8-7-5 (2′- | muHTT SNP |
| | CAGACUUCC | | *G*C*A*C*A*G*A*mCmU | | XXXXXXOOOO | OMe-DNA-2′- | 362307 |
| | | | mUmCmC | | | OMe); | |
| | | | | | | stereorandom PS; | |
| | | | | | | PO in the wings | |
| WV-927 | CACAAGGGCAC | 1743 | mCmAmCmAmAmGmG*G* | 2095 | OOOOOOXXX | ASO24 7-7-6 (2′- | muHTT SNP |
| | AGACUUCCA | | C*A*C*A*G*A*mCmUmU | | XXXXXOOOOO | OMe-DNA-2′- | 362307 |
| | | | mCmCmA | | | OMe); | |
| | | | | | | stereorandom PS; | |
| | | | | | | PO in the wings | |
| WV-928 | ACAAGGGCACA | 1744 | mAmCmAmAmGmG*G*C* | 2096 | OOOOOXXXX | ASO25 6-7-5 (2′- | muHTT SNP |
| | GACUUCCAA | | A*C*A*G*A*mCmUmUmC | | XXXXOOOOOO | OMe-DNA-2′- | 362307 |
| | | | mCmAmA | | | OMe); | |
| | | | | | | stereorandom PS; | |
| | | | | | | PO in the wings | |
| WV-929 | CAAGGGCACAG | 1745 | mCmAmAmGmG*G*C*A*C | 2097 | OOOOXXXXX | ASO26 5-7-8 (2′- | muHTT SNP |
| | ACUUCCAAA | | *A*G*A*mCmUmUmCmCm | | XXXOOOOOOO | OMe-DNA-2′- | 362307 |
| | | | AmAmA | | | OMe); | |
| | | | | | | stereorandom PS; | |
| | | | | | | PO in the wings | |
| WV-930 | GGGCACAAGG | 1746 | mGmGmGmCmA*C*A*A*G | 2098 | OOOOXXXXX | ASO27 5-10-5 | muHTT SNP |
| | GCACAGACUU | | *G*G*C*A*C*A*mGmAmC | | XXXXXXOOOO | (2′-OMe-DNA- | 362307 |
| | | | mUmU | | | 2′-OMe); | |
| | | | | | | stereorandom PS; | |
| | | | | | | PO in the wings | |
| WV-931 | GGCACAAGGGC | 1747 | mGmGmCmAmC*A*A*G*G | 2099 | OOOOXXXXX | ASO28 5-10-5 | muHTT SNP |
| | ACAGACUUC | | *G*C*A*C*A*G*mAmCmU | | XXXXXXOOOO | (2′-OMe-DNA- | 362307 |
| | | | mUmC | | | 2′-OMe); | |
| | | | | | | stereorandom PS; | |
| | | | | | | PO in the wings | |
| WV-932 | GCACAAGGGCA | 1748 | mGmCmAmCmA*A*G*G*G | 2100 | OOOOXXXXX | ASO29 5-10-5 | muHTT SNP |
| | CAGACUUCC | | *C*A*C*A*G*A*mCmUmU | | XXXXXXOOOO | (2′-OMe-DNA- | 362307 |
| | | | mCmC | | | 2′-OMe); | |
| | | | | | | stereorandom PS; | |
| | | | | | | PO in the wings | |
| WV-933 | CACAAGGGCAC | 1749 | mCmAmCmAmA*G*G*G*C | 2101 | OOOOXXXXX | ASO30 5-10-5 | muHTT SNP |
| | AGACUUCCA | | *A*C*A*G*A*C*mUmUmC | | XXXXXXOOOO | (2′-OMe-DNA- | 362307 |
| | | | mCmA | | | 2′-OMe); | |
| | | | | | | stereorandom PS; | |
| | | | | | | PO in the wings | |
| WV-934 | ACAAGGGCACA | 1750 | mAmCmAmAmG*G*G*C*A | 2102 | OOOOXXXXX | ASO31 5-10-5 | muHTT SNP |
| | GACTUCCAA | | *C*A*G*A*C*T*mUmCmC | | XXXXXXOOOO | (2′-OMe-DNA- | 362307 |
| | | | mAmA | | | 2′-OMe); | |
| | | | | | | stereorandom PS; | |
| | | | | | | PO in the wings | |
| WV-935 | CAAGGGCACAG | 1751 | mCmAmAmGmG*G*C*A*C | 2103 | OOOOXXXXX | ASO32 5-10-5 | muHTT SNP |
| | ACTTCCAAA | | *A*G*A*C*T*T*mCmCmA | | XXXXXXOOOO | (2′-OMe-DNA- | 362307 |
| | | | mAmA | | | 2′-OMe); | |
| | | | | | | stereorandom PS; | |
| | | | | | | PO in the wings | |
| WV-936 | GGGCACAAGG | 1752 | G*SG*SG*SC*SA*SC*SA* | 2104 | SSSSSSSSSSSS | ASO33 | muHTT SNP |
| | GCACAGACTT | | SA*SG*SG*SG*SC*SA*SC | | SRSSSSS | Stereopure DNA; | 362307 |
| | | | *RA*SG*SA*SC*ST*ST | | | One Rp; position | |
| | | | | | | 14 | |
| WV-937 | GGCACAAGGGC | 1753 | G*SG*SC*SA*SC*SA*SA* | 2105 | SSSSSSSSSSSS | ASO34 | muHTT SNP |
| | ACAGACTTC | | SG*SG*SG*SC*SA*SC*RA | | RSSSSSS | Stereopure DNA; | 362307 |
| | | | *SG*SA*SC*ST*ST*SC | | | One Rp; position | |
| | | | | | | 13 | |
| WV-938 | GCACAAGGGCA | 1754 | G*SC*SA*SC*SA*SA*SG* | 2106 | SSSSSSSSSSSR | ASO35 | muHTT SNP |
| | CAGACTTCC | | SG*SG*SC*SA*SC*RA*SG | | SSSSSSS | Stereopure DNA; | 362307 |
| | | | *SA*SC*ST*ST*SC*SC | | | One Rp; position | |
| | | | | | | 12 | |
| WV-939 | CACAAGGGCAC | 1755 | C*SA*SC*SA*SA*SG*SG* | 2107 | SSSSSSSSSSRS | ASO36 | muHTT SNP |
| | AGACTTCCA | | SG*SC*SA*SC*RA*SG*SA | | SSSSSSS | Stereopure DNA; | 362307 |
| | | | *SC*ST*ST*SC*SC*SA | | | One Rp; position | |
| | | | | | | 11 | |
| WV-940 | ACAAGGGCACA | 1756 | A*SC*SA*SA*SG*SG*SG* | 2108 | SSSSSSSSSRSS | ASO37 | muHTT SNP |
| | GACTTCCAA | | SC*SA*SC*RA*SG*SA*SC | | SSSSSSS | Stereopure DNA; | 362307 |
| | | | *ST*ST*SC*SC*SA*SA | | | One Rp; position | |
| | | | | | | 10 | |
| WV-941 | CAAGGGCACAG | 1757 | C*SA*SA*SG*SG*SG*SC* | 2109 | SSSSSSSSRSSS | ASO38 | muHTT SNP |
| | ACTTCCAAA | | SA*SC*RA*SG*SA*SC*ST | | SSSSSSS | Stereopure DNA; | 362307 |
| | | | *ST*SC*SC*SA*SA*SA | | | One Rp; position 9 | |
| WV-944 | UUUGGAAGUC | 1758 | rUrUrUrGrGrArArGrUrCrUr | 2110 | OOOOOOOOO | HTT-rs362307 | Huntington |
| | UGCGCCCUUGU | | GrCrGrCrCrCrUrUrGrUrGrC | | OOOOOOOOO | human | |
| | GCCC | | rCrC | | OOOOOO | | |
| WV-945 | UUUGGAAGUC | 1759 | rUrUrUrGrGrArArGrUrCrUr | 2111 | OOOOOOOOO | HTT-rs362307 | Huntington |
| | UGUGCCCUUGU | | GrUrGrCrCrCrUrUrGrUrGrC | | OOOOOOOOO | human | |
| | GCCC | | rCrC | | OOOOOO | | |
| WV-948 | GAGCAGCTGCA | 1760 | G*A*G*C*A*G*C*T*G*C* | 2112 | XXXXXXXXX | HTT-rs362306 | HTT-rs362306 |
| | ACCTGGCAA | | A*A*C*C*T*G*G*C*A*A | | XXXXXXXXXX | | |
| WV-949 | GGGCCAACAGC | 1761 | G*G*G*C*C*A*A*C*A*G* | 2113 | XXXXXXXXX | HTT-rs362268 | HTT-rs362268 |
| | CAGCCTGCA | | C*C*A*G*C*C*T*G*C*A | | XXXXXXXXXX | | |
| WV-950 | GGUUGUUGCC | 1762 | rGrGrUrUrGrUrUrGrCrCrAr | 2114 | OOOOOOOOO | | HTT-rs362306 |
| | AGGUUACAGC | | GrGrUrUrArCrArGrCrUrGrC | | OOOOOOOOO | | |
| | UGCUC | | rUrC | | OOOOOO | | |
| WV-951 | GGUUGUUGCC | 1763 | rGrGrUrUrGrUrUrGrCrCrAr | 2115 | OOOOOOOOO | | HTT-rs362306 |
| | AGGUUGCAGC | | GrGrUrUrGrCrArGrCrUrGrC | | OOOOOOOOO | | |
| | UGCUC | | rUrC | | OOOOOO | | |
| WV-952 | GAGCAGCTGCA | 1764 | G*SA*SG*SC*SA*SG*SC* | 2116 | SSSSSSSSSSRS | Stereopure PS | HTT-rs362306 |
| | ACCTGGCAA | | ST*SG*SC*SA*RA*SC*SC* | | SSSSSSS | DNA; One Rp at | |
| | | | ST*SG*SG*SC*SA*SA | | | position 11 | |
| WV-953 | AGCAGCTGCAA | 1765 | A*SG*SC*SA*SG*SC*ST*S | 2117 | SSSSSSSSSRSS | Stereopure PS | HTT-rs362306 |
| | CCTGGCAAC | | G*SC*SA*RA*SC*SC*ST*S | | SSSSSSS | DNA; One Rp at | |
| | | | G*SG*SC*SA*SA*SC | | | position 10 | |
| WV-954 | GCAGCTGCAAC | 1766 | G*SC*SA*SG*SC*ST*SG*S | 2118 | SSSSSSSSRSSS | Stereopure PS | HTT-rs362306 |
| | CTGGCAACA | | C*SA*RA*SC*SC*ST*SG*S | | SSSSSSS | DNA; One Rp at | |
| | | | G*SC*SA*SA*SC*SA | | | position 9 | |
| WV-955 | CAGCTGCAACC | 1767 | C*SA*SG*SC*ST*SG*SC*S | 2119 | SSSSSSSRSSSS | Stereopure PS | HTT-rs362306 |
| | TGGCAACAA | | A*RA*SC*SC*ST*SG*SG* | | SSSSSSS | DNA; One Rp at | |
| | | | SC*SA*SA*SC*SA*SA | | | position 8 | |
| WV-956 | AGCTGCAACCT | 1768 | A*SG*SC*ST*SG*SC*SA* | 2120 | SSSSSSRSSSSS | Stereopure PS | HTT-rs362306 |
| | GGCAACAAC | | RA*SC*SC*ST*SG*SG*SC* | | SSSSSSS | DNA; One Rp at | |
| | | | SA*SA*SC*SA*SA*SC | | | position 7 | |
| WV-957 | GCTGCAACCTG | 1769 | G*SC*ST*SG*SC*SA*RA* | 2121 | SSSSSRSSSSSS | Stereopure PS | HTT-rs362306 |
| | GCAACAACC | | SC*SC*ST*SG*SG*SC*SA* | | SSSSSSS | DNA; One Rp at | |
| | | | SA*SC*SA*SA*SC*SC | | | position 6 | |
| WV-958 | CCUCCUGCAGG | 1770 | rCrCrUrCrCrUrGrCrArGrGrC | 2122 | OOOOOOOOO | | HTT-rs362268 |
| | CUGGGUGUUG | | rUrGrGrGrUrGrUrUrGrGrCr | | OOOOOOOOO | | |
| | GCCC | | CrC | | OOOOOO | | |
| WV-959 | CCUCCUGCAGG | 1771 | rCrCrUrCrCrUrGrCrArGrGrC | 2123 | OOOOOOOOO | | HTT-rs362268 |
| | CUGGCUGUUG | | rUrGrGrCrUrGrUrUrGrGrCr | | OOOOOOOOO | | |
| | GCCC | | CrC | | OOOOOO | | |
| WV-960 | GGGCCAACAGC | 1772 | G*SG*SG*SC*SC*SA*SA* | 2124 | SSSSSSSSSSRS | Stereopure PS | HTT-rs362268 |
| | CAGCCTGCA | | SC*SA*SG*SC*RC*SA*SG | | SSSSSSS | DNA; One Rp at | |
| | | | *SC*SC*ST*SG*SC*SA | | | position 11 | |
| WV-961 | GGCCAACAGCC | 1773 | G*SG*SC*SC*SA*SA*SC*S | 2125 | SSSSSSSSSRSS | Stereopure PS | HTT-rs362268 |
| | AGCCTGCAG | | A*SG*SC*RC*SA*SG*SC* | | SSSSSSS | DNA; One Rp at | |
| | | | SC*ST*SG*SC*SA*SG | | | position 10 | |
| WV-962 | GCCAACAGCCA | 1774 | G*SC*SC*SA*SA*SC*SA*S | 2126 | SSSSSSSSRSSS | Stereopure PS | HTT-rs362268 |
| | GCCTGCAGG | | G*SC*RC*SA*SG*SC*SC* | | SSSSSSS | DNA; One Rp at | |
| | | | ST*SG*SC*SA*SG*SG | | | position 9 | |
| WV-963 | CCAACAGCCAG | 1775 | C*SC*SA*SA*SC*SA*SG*S | 2127 | SSSSSSSRSSSS | Stereopure PS | HTT-rs362268 |
| | CCTGCAGGA | | C*RC*SA*SG*SC*SC*ST*S | | SSSSSSS | DNA; One Rp at | |
| | | | G*SC*SA*SG*SG*SA | | | position 8 | |
| WV-964 | CAACAGCCAGC | 1776 | C*SA*SA*SC*SA*SG*SC* | 2128 | SSSSSSRSSSSS | Stereopure PS | HTT-rs362268 |
| | CTGCAGGAG | | RC*SA*SG*SC*SC*ST*SG* | | SSSSSSS | DNA; One Rp at | |
| | | | SC*SA*SG*SG*SA*SG | | | position 7 | |
| WV-965 | AACAGCCAGCC | 1777 | A*SA*SC*SA*SG*SC*RC* | 2129 | SSSSSRSSSSSS | Stereopure PS | HTT-rs362268 |
| | TGCAGGAGG | | SA*SG*SC*SC*ST*SG*SC* | | SSSSSSS | DNA; One Rp at | |
| | | | SA*SG*SG*SA*SG*SG | | | position 6 | |
| WV-973 | GGCCUUUCACU | 1778 | rGrGrCrCrUrUrUrCrArCrUr | 2130 | OOOOOOOOO | siRNA (+control | Htt |
| | ACUCCUACTT | | ArCrUrCrCrUrArCTT | | OOOOOOOOO | for Renilla | |
| | | | | | OO | luciferase in | |
| | | | | | | psiCHECK2 | |
| | | | | | | plasmid) | |
| | | | | | | antisense strand | |
| WV-974 | GUAGGAGUAG | 1779 | rGrUrArGrGrArGrUrArGrUr | 2131 | OOOOOOOOO | siRNA (+control | Htt SNP |
| | UGAAAGGCCTT | | GrArArArGrGrCrCTT | | OOOOOOOOO | for Renilla | rs362268 |
| | | | | | OO | luciferase in | |
| | | | | | | psiCHECK2 | |
| | | | | | | plasmid) sense | |
| | | | | | | strand | |
| WV-975 | GTAGGAGTAGT | 1780 | G*T*A*G*G*A*G*T*A*G* | 2132 | XXXXXXXXX | ASO (+control | Htt SNP |
| | GAAAGGCCA | | T*G*A*A*A*G*G*C*C*A | | XXXXXXXXXX | for Renilla | rs362268 |
| | | | | | | luciferase in | |
| | | | | | | psiCHECK2 | |
| | | | | | | plasmid) | |
| WV-982 | GCAGGGCACAA | 1781 | G*SC*SA*SG*SG*SG*SC* | 2133 | SSSSSSSSSSSS | Htt seq 307 | Htt rs362307 |
| | GGGCACAGA | | SA*SC*SA*SA*SG*SG*SG | | SSSSRSS | expanding 3 nt | |
| | | | *SC*SA*SC*RA*SG*SA | | | towards 3′ | |
| | | | | | | example 3 | |
| WV-983 | CAGGGCACAAG | 1782 | C*SA*SG*SG*SG*SC*SA* | 2134 | SSSSSSSSSSSS | Htt seq 307 | Htt rs362307 |
| | GGCACAGAC | | SC*SA*SA*SG*SG*SG*SC | | SSSRSSS | expanding 3 nt | |
| | | | *SA*SC*RA*SG*SA*SC | | | towards 3′ | |
| | | | | | | example 2 | |
| WV-984 | AGGGCACAAG | 1783 | A*SG*SG*SG*SC*SA*SC* | 2135 | SSSSSSSSSSSS | Htt seq 307 | Htt rs362307 |
| | GGCACAGACT | | SA*SA*SG*SG*SG*SC*SA | | SSRSSSS | expanding 3 nt | |
| | | | *SC*RA*SG*SA*SC*ST | | | towards 3′ | |
| | | | | | | example 1 | |
| WV-985 | AAGGGCACAG | 1784 | A*SA*SG*SG*SG*SC*SA* | 2136 | SSSSSSSRSSSS | Htt seq 307 | Htt rs362307 |
| | ACTTCCAAAG | | SC*RA*SG*SA*SC*ST*ST* | | SSSSSSS | expanding 3 nt | |
| | | | SC*SC*SA*SA*SA*SG | | | towards 5′ | |
| | | | | | | example 1 | |
| WV-986 | AGGGCACAGAC | 1785 | A*SG*SG*SG*SC*SA*SC* | 2137 | SSSSSSRSSSSS | Htt seq 307 | Htt rs362307 |
| | TTCCAAAGG | | RA*SG*SA*SC*ST*ST*SC* | | SSSSSSS | expanding 3 nt | |
| | | | SC*SA*SA*SA*SG*SG | | | towards 5′ | |
| | | | | | | example 2 | |
| WV-987 | GGGCACAGACT | 1786 | G*SG*SG*SC*SA*SC*RA* | 2138 | SSSSSRSSSSSS | Htt seq 307 | Htt rs362307 |
| | TCCAAAGGC | | SG*SA*SC*ST*ST*SC*SC* | | SSSSSSS | expanding 3 nt | |
| | | | SA*SA*SA*SG*SG*SC | | | towards 5′ | |
| | | | | | | example 3 | |
| WV-1001 | GAGCAGCTGCA | 1787 | G*A*G*C*A*G*C*T*G*C* | 2139 | XXXXXXXXX | All DNA; | HTT-rs362306 |
| | ACCTGGCAA | | A*A*C*C*T*G*G*C*A*A | | XXXXXXXXXX | stereorandom PS | |
| WV-1002 | AGCAGCTGCAA | 1788 | A*G*C*A*G*C*T*G*C*A* | 2140 | XXXXXXXXX | All DNA; | HTT-rs362306 |
| | CCTGGCAAC | | A*C*C*T*G*G*C*A*A*C | | XXXXXXXXXX | stereorandom PS | |
| WV-1003 | GCAGCTGCAAC | 1789 | G*C*A*G*C*T*G*C*A*A* | 2141 | XXXXXXXXX | All DNA; | HTT-rs362306 |
| | CTGGCAACA | | C*C*T*G*G*C*A*A*C*A | | XXXXXXXXXX | stereorandom PS | |
| WV-1004 | CAGCTGCAACC | 1790 | C*A*G*C*T*G*C*A*A*C* | 2142 | XXXXXXXXX | All DNA; | HTT-rs362306 |
| | TGGCAACAA | | C*T*G*G*C*A*A*C*A*A | | XXXXXXXXXX | stereorandom PS | |
| WV-1005 | AGCTGCAACCT | 1791 | A*G*C*T*G*C*A*A*C*C* | 2143 | XXXXXXXXX | All DNA; | HTT-rs362306 |
| | GGCAACAAC | | T*G*G*C*A*A*C*A*A*C | | XXXXXXXXXX | stereorandom PS | |
| WV-1006 | GCTGCAACCTG | 1792 | G*C*T*G*C*A*A*C*C*T* | 2144 | XXXXXXXXX | All DNA; | HTT-rs362306 |
| | GCAACAACC | | G*G*C*A*A*C*A*A*C*C | | XXXXXXXXXX | stereorandom PS | |
| WV-1007 | GAGCAGCTGCA | 1793 | mG*mA*mG*mC*mA*G*C | 2145 | XXXXXXXXX | 5-15 (2′-OMe- | HTT-rs362306 |
| | ACCTGGCAA | | *T*G*C*A*A*C*C*T*G*G | | XXXXXXXXXX | DNA); | |
| | | | *C*A*A | | | stereorandom PS | |
| WV-1008 | AGCAGCTGCAA | 1794 | mA*mG*mC*mA*mG*C*T* | 2146 | XXXXXXXXX | 5-15 (2′-OMe- | HTT-rs362306 |
| | CCTGGCAAC | | G*C*A*A*C*C*T*G*G*C* | | XXXXXXXXXX | DNA); | |
| | | | A*A*C | | | stereorandom PS | |
| WV-1009 | GCAGCTGCAAC | 1795 | mG*mC*mA*mG*mC*T*G* | 2147 | XXXXXXXXX | 5-15 (2′-OMe- | HTT-rs362306 |
| | CTGGCAACA | | C*A*A*C*C*T*G*G*C*A* | | XXXXXXXXXX | DNA); | |
| | | | A*C*A | | | stereorandom PS | |
| WV-1010 | CAGCUGCAACC | 1796 | mC*mA*mG*mC*mU*G*C* | 2148 | XXXXXXXXX | 5-15 (2′-OMe- | HTT-rs362306 |
| | TGGCAACAA | | A*A*C*C*T*G*G*C*A*A* | | XXXXXXXXXX | DNA); | |
| | | | C*A*A | | | stereorandom PS | |
| WV-1011 | AGCUGCAACCT | 1797 | mA*mG*mC*mU*mG*C*A | 2149 | XXXXXXXXX | 5-15 (2′-OMe- | HTT-rs362306 |
| | GGCAACAAC | | *A*C*C*T*G*G*C*A*A*C | | XXXXXXXXXX | DNA); | |
| | | | *A*A*C | | | stereorandom PS | |
| WV-1012 | GCUGCAACCTG | 1798 | mG*mC*mU*mG*mC*A*A | 2150 | XXXXXXXXX | 5-15 (2′-OMe- | HTT-rs362306 |
| | GCAACAACC | | *C*C*T*G*G*C*A*A*C*A | | XXXXXXXXXX | DNA); | |
| | | | *A*C*C | | | stereorandom PS | |
| WV-1013 | GAGCAGCTGCA | 1799 | mG*mA*mG*mC*mA*G*C | 2151 | XXXXXXXXX | 5-10-5 (2′-OMe- | HTT-rs362306 |
| | ACCTGGCAA | | *T*G*C*A*A*C*C*T*mG* | | XXXXXXXXXX | DNA-2′-OMe); | |
| | | | mG*mC*mA*mA | | | stereorandom PS | |
| WV-1014 | AGCAGCTGCAA | 1800 | mA*mG*mC*mA*mG*C*T* | 2152 | XXXXXXXXX | 5-10-5 (2′-OMe- | HTT-rs362306 |
| | CCTGGCAAC | | G*C*A*A*C*C*T*G*mG*m | | XXXXXXXXXX | DNA-2′-OMe); | |
| | | | C*mA*mA*mC | | | stereorandom PS | |
| WV-1015 | GCAGCTGCAAC | 1801 | mG*mC*mA*mG*mC*T*G* | 2153 | XXXXXXXXX | 5-10-5 (2′-OMe- | HTT-rs362306 |
| | CTGGCAACA | | C*A*A*C*C*T*G*G*mC*m | | XXXXXXXXXX | DNA-2′-OMe); | |
| | | | A*mA*mC*mA | | | stereorandom PS | |
| WV-1016 | CAGCUGCAACC | 1802 | mC*mA*mG*mC*mU*G*C* | 2154 | XXXXXXXXX | 5-10-5 (2′-OMe- | HTT-rs362306 |
| | TGGCAACAA | | A*A*C*C*T*G*G*C*mA*m | | XXXXXXXXXX | DNA-2′-OMe); | |
| | | | A*mC*mA*mA | | | stereorandom PS | |
| WV-1017 | AGCUGCAACCT | 1803 | mA*mG*mC*mU*mG*C*A | 2155 | XXXXXXXXX | 5-10-5 (2′-OMe- | HTT-rs362306 |
| | GGCAACAAC | | *A*C*C*T*G*G*C*A*mA* | | XXXXXXXXXX | DNA-2′-OMe); | |
| | | | mC*mA*mA*mC | | | stereorandom PS | |
| WV-1018 | GCUGCAACCTG | 1804 | mG*mC*mU*mG*mC*A*A | 2156 | XXXXXXXXX | 5-10-5 (2′-OMe- | HTT-rs362306 |
| | GCAACAACC | | *C*C*T*G*G*C*A*A*mC* | | XXXXXXXXXX | DNA-2′-OMe); | |
| | | | mA*mA*mC*mC | | | stereorandom PS | |
| WV-1019 | GAGCAGCTGCA | 1805 | mG*mA*mG*mC*mA*mG* | 2157 | XXXXXXXXX | 7-7-6 (2′-OMe- | HTT-rs362306 |
| | ACCUGGCAA | | mC*T*G*C*A*A*C*C*mU* | | XXXXXXXXX | DNA-2′-OMe); | |
| | | | mG*mG*mC*mA*mA | | | stereorandom PS | |
| WV-1020 | GAGCAGCTGCA | 1806 | mGmAmGmCmAmGmC*T* | 2158 | OOOOOOXXX | 7-7-6 (2′-OMe- | HTT-rs362306 |
| | ACCUGGCAA | | G*C*A*A*C*C*mUmGmG | | XXXXXOOOOO | DNA-2′-OMe); | |
| | | | mCmAmA | | | stereorandom PS; | |
| | | | | | | PO in wings | |
| WV-1021 | AGCAGCTGCAA | 1807 | mA*mG*mC*mA*mG*mC* | 2159 | XXXXXXXXX | 6-7-5 (2′-OMe- | HTT-rs362306 |
| | CCTGGCAAC | | T*G*C*A*A*C*C*T*G*mG | | XXXXXXXXXX | DNA-2′-OMe); | |
| | | | *mC*mA*mA*mC | | | stereorandom PS | |
| WV-1022 | AGCAGCTGCAA | 1808 | mAmGmCmAmGmC*T*G* | 2160 | OOOOOXXXX | 6-7-5 (2′-OMe- | HTT-rs362306 |
| | CCTGGCAAC | | C*A*A*C*C*T*G*mGmCm | | XXXXXXOOOO | DNA-2′-OMe); | |
| | | | AmAmC | | | stereorandom PS; | |
| | | | | | | PO in the wings | |
| WV-1023 | GCAGCTGCAAC | 1809 | mG*mC*mA*mG*mC*T*G* | 2161 | XXXXXXXXX | 5-7-8 (2′-OMe- | HTT-rs362306 |
| | CUGGCAACA | | C*A*A*C*C*mU*mG*mG* | | XXXXXXXXXX | DNA-2′-OMe); | |
| | | | mC*mA*mA*mC*mA | | | stereorandom PS | |
| WV-1024 | GCAGCTGCAAC | 1810 | mGmCmAmGmC*T*G*C*A | 2162 | OOOOXXXXX | 5-7-8 (2′-OMe- | HTT-rs362306 |
| | CUGGCAACA | | *A*C*C*mUmGmGmCmAm | | XXXOOOOOOO | DNA-2′-OMe); | |
| | | | AmCmA | | | stereorandom PS; | |
| | | | | | | PO in the wings | |
| WV-1025 | GAGCAGCTGCA | 1811 | mGmAmGmCmA*G*C*T*G | 2163 | OOOOXXXXX | 5-10-5 (2′-OMe- | HTT-rs362306 |
| | ACCTGGCAA | | *C*A*A*C*C*T*mGmGmC | | XXXXXXOOOO | DNA-2′-OMe); | |
| | | | mAmA | | | stereorandom PS; | |
| | | | | | | PO in the wings | |
| WV-1026 | AGCAGCTGCAA | 1812 | mAmGmCmAmG*C*T*G*C | 2164 | OOOOXXXXX | 5-10-5 (2′-OMe- | HTT-rs362306 |
| | CCTGGCAAC | | *A*A*C*C*T*G*mGmCmA | | XXXXXXOOOO | DNA-2′-OMe); | |
| | | | mAmC | | | stereorandom PS; | |
| | | | | | | PO in the wings | |
| WV-1027 | GCAGCTGCAAC | 1813 | mGmCmAmGmCT*G*C*A* | 2165 | OOOOOXXXX | 5-10-5 (2′-OMe- | HTT-rs362306 |
| | CTGGCAACA | | A*C*C*T*G*G*mCmAmA | | XXXXXXOOOO | DNA-2′-OMe); | |
| | | | mCmA | | | stereorandom PS; | |
| | | | | | | PO in the wings | |
| WV-1028 | CAGCUGCAACC | 1814 | mCmAmGmCmU*G*C*A*A | 2166 | OOOOXXXXX | 5-10-5 (2′-OMe- | HTT-rs362306 |
| | TGGCAACAA | | *C*C*T*G*G*C*mAmAmC | | XXXXXXOOOO | DNA-2′-OMe); | |
| | | | mAmA | | | stereorandom PS; | |
| | | | | | | PO in the wings | |
| WV-1029 | AGCUGCAACCT | 1815 | mAmGmCmUmG*C*A*A*C | 2167 | OOOOXXXXX | 5-10-5 (2′-OMe- | HTT-rs362306 |
| | GGCAACAAC | | *C*T*G*G*C*A*mAmCmA | | XXXXXXOOOO | DNA-2′-OMe); | |
| | | | mAmC | | | stereorandom PS; | |
| | | | | | | PO in the wings | |
| WV-1030 | GCUGCAACCTG | 1816 | mGmCmUmGmC*A*A*C*C | 2168 | OOOOXXXXX | 5-10-5 (2′-OMe- | HTT-rs362306 |
| | GCAACAACC | | *T*G*G*C*A*A*mCmAmA | | XXXXXXOOOO | DNA-2′-OMe); | |
| | | | mCmC | | | stereorandom PS; | |
| | | | | | | PO in the wings | |
| WV-1031 | GGGCCAACAGC | 1817 | G*G*G*C*C*A*A*C*A*G* | 2169 | XXXXXXXXX | All DNA; | HTT-rs362268 |
| | CAGCCTGCA | | C*C*A*G*C*C*T*G*C*A | | XXXXXXXXXX | stereorandom PS | |
| WV-1032 | GGCCAACAGCC | 1818 | G*G*C*C*A*A*C*A*G*C* | 2170 | XXXXXXXXX | All DNA; | HTT-rs362268 |
| | AGCCTGCAG | | C*A*G*C*C*T*G*C*A*G | | XXXXXXXXXX | stereorandom PS | |
| WV-1033 | GCCAACAGCCA | 1819 | G*C*C*A*A*C*A*G*C*C* | 2171 | XXXXXXXXX | All DNA; | HTT-rs362268 |
| | GCCTGCAGG | | A*G*C*C*T*G*C*A*G*G | | XXXXXXXXXX | stereorandom PS | |
| WV-1034 | CCAACAGCCAG | 1820 | C*C*A*A*C*A*G*C*C*A* | 2172 | XXXXXXXXX | All DNA; | HTT-rs362268 |
| | CCTGCAGGA | | G*C*C*T*G*C*A*G*G*A | | XXXXXXXXXX | stereorandom PS | |
| WV-1035 | CAACAGCCAGC | 1821 | C*A*A*C*A*G*C*C*A*G* | 2173 | XXXXXXXXX | All DNA; | HTT-rs362268 |
| | CTGCAGGAG | | C*C*T*G*C*A*G*G*A*G | | XXXXXXXXXX | stereorandom PS | |
| WV-1036 | AACAGCCAGCC | 1822 | A*A*C*A*G*C*C*A*G*C* | 2174 | XXXXXXXXX | All DNA; | HTT-rs362268 |
| | TGCAGGAGG | | C*T*G*C*A*G*G*A*G*G | | XXXXXXXXXX | stereorandom PS | |
| WV-1037 | GGGCCAACAGC | 1823 | mG*mG*mG*mC*mC*A*A | 2175 | XXXXXXXXX | 5-15 (2′-OMe- | HTT-rs362268 |
| | CAGCCTGCA | | *C*A*G*C*C*A*G*C*C*T | | XXXXXXXXXX | DNA); | |
| | | | *G*C*A | | | stereorandom PS | |
| WV-1038 | GGCCAACAGCC | 1824 | mG*mG*mC*mC*mA*A*C* | 2176 | XXXXXXXXX | 5-15 (2′-OMe- | HTT-rs362268 |
| | AGCCTGCAG | | A*G*C*C*A*G*C*C*T*G* | | XXXXXXXXXX | DNA); | |
| | | | C*A*G | | | stereorandom PS | |
| WV-1039 | GCCAACAGCCA | 1825 | mG*mC*mC*mA*mA*C*A* | 2177 | XXXXXXXXX | 5-15 (2′-OMe- | HTT-rs362268 |
| | GCCTGCAGG | | G*C*C*A*G*C*C*T*G*C* | | XXXXXXXXXX | DNA); | |
| | | | A*G*G | | | stereorandom PS | |
| WV-1040 | CCAACAGCCAG | 1826 | mC*mC*mA*mA*mC*A*G* | 2178 | XXXXXXXXX | 5-15 (2′-OMe- | HTT-rs362268 |
| | CCTGCAGGA | | C*C*A*G*C*C*T*G*C*A* | | XXXXXXXXXX | DNA); | |
| | | | G*G*A | | | stereorandom PS | |
| WV-1041 | CAACAGCCAGC | 1827 | mC*mA*mA*mC*mA*G*C* | 2179 | XXXXXXXXX | 5-15 (2′-OMe- | HTT-rs362268 |
| | CTGCAGGAG | | C*A*G*C*C*T*G*C*A*G* | | XXXXXXXXXX | DNA); | |
| | | | G*A*G | | | stereorandom PS | |
| WV-1042 | AACAGCCAGCC | 1828 | mA*mA*mC*mA*mG*C*C* | 2180 | XXXXXXXXX | 5-15 (2′-OMe- | HTT-rs362268 |
| | TGCAGGAGG | | A*G*C*C*T*G*C*A*G*G* | | XXXXXXXXXX | DNA); | |
| | | | A*G*G | | | stereorandom PS | |
| WV-1043 | GGGCCAACAGC | 1829 | mG*mG*mG*mC*mC*A*A | 2181 | XXXXXXXXX | 5-10-5 (2′-OMe- | HTT-rs362268 |
| | CAGCCUGCA | | *C*A*G*C*C*A*G*C*mC* | | XXXXXXXXXX | DNA-2′-OMe); | |
| | | | mU*mG*mC*mA | | | stereorandom PS | |
| WV-1044 | GGCCAACAGCC | 1830 | mG*mG*mC*mC*mA*A*C* | 2182 | XXXXXXXXX | 5-10-5 (2′-OMe- | HTT-rs362268 |
| | AGCCUGCAG | | A*G*C*C*A*G*C*C*mU* | | XXXXXXXXXX | DNA-2′-OMe); | |
| | | | mG*mC*mA*mG | | | stereorandom PS | |
| WV-1045 | GCCAACAGCCA | 1831 | mG*mC*mC*mA*mA*C*A* | 2183 | XXXXXXXXX | 5-10-5 (2′-OMe- | HTT-rs362268 |
| | GCCTGCAGG | | G*C*C*A*G*C*C*T*mG*m | | XXXXXXXXXX | DNA-2′-OMe); | |
| | | | C*mA*mG*mG | | | stereorandom PS | |
| WV-1046 | CCAACAGCCAG | 1832 | mC*mC*mA*mA*mC*A*G* | 2184 | XXXXXXXXX | 5-10-5 (2′-OMe- | HTT-rs362268 |
| | CCTGCAGGA | | C*C*A*G*C*C*T*G*mC*m | | XXXXXXXXXX | DNA-2′-OMe); | |
| | | | A*mG*mG*mA | | | stereorandom PS | |
| WV-1047 | CAACAGCCAGC | 1833 | mC*mA*mA*mC*mA*G*C* | 2185 | XXXXXXXXX | 5-10-5 (2′-OMe- | HTT-rs362268 |
| | CTGCAGGAG | | C*A*G*C*C*T*G*C*mA*m | | XXXXXXXXXX | DNA-2′-OMe); | |
| | | | G*mG*mA*mG | | | stereorandom PS | |
| WV-1048 | AACAGCCAGCC | 1834 | mA*mA*mC*mA*mG*C*C* | 2186 | XXXXXXXXX | 5-10-5 (2′-OMe- | HTT-rs362268 |
| | TGCAGGAGG | | A*G*C*C*T*G*C*A*mG*m | | XXXXXXXXXX | DNA-2′-OMe); | |
| | | | G*mA*mG*mG | | | stereorandom PS | |
| WV-1049 | GGGCCAACAGC | 1835 | mG*mG*mG*mC*mC*mA* | 2187 | XXXXXXXXX | 7-7-6 (2′-OMe- | HTT-rs362268 |
| | CAGCCUGCA | | mA*C*A*G*C*C*A*G*mC | | XXXXXXXXXX | DNA-2′-OMe); | |
| | | | *mC*mU*mG*mC*mA | | | stereorandom PS | |
| WV-1050 | GGGCCAACAGC | 1836 | mGmGmGmCmCmAmA*C* | 2188 | OOOOOOXXX | 7-7-6 (2′-OMe- | HTT-rs362268 |
| | CAGCCUGCA | | A*G*C*C*A*G*mCmCmU | | XXXXXOOOOO | DNA-2′-OMe); | |
| | | | mGmCmA | | | stereorandom PS; | |
| | | | | | | PO in wings | |
| WV-1051 | GGCCAACAGCC | 1837 | mG*mG*mC*mC*mA*mA* | 2189 | XXXXXXXXX | 6-7-5 (2′-OMe- | HTT-rs362268 |
| | AGCCUGCAG | | C*A*G*C*C*A*G*C*C*mU | | XXXXXXXXX | DNA-2′-OMe); | |
| | | | *mG*mC*mA*mG | | | stereorandom PS | |
| WV-1052 | GGCCAACAGCC | 1838 | mGmGmCmCmAmA*C*A* | 2190 | OOOOOXXXX | 6-7-5 (2′-OMe- | HTT-rs362268 |
| | AGCCUGCAG | | G*C*C*A*G*C*C*mUmGm | | XXXXXXOOOO | DNA-2′-OMe); | |
| | | | CmAmG | | | stereorandom PS; | |
| | | | | | | PO in the wings | |
| WV-1053 | GCCAACAGCCA | 1839 | mG*mC*mC*mA*mA*C*A* | 2191 | XXXXXXXXX | 5-7-8 (2′-OMe- | HTT-rs362268 |
| | GCCUGCAGG | | G*C*C*A*G*mC*mC*mU* | | XXXXXXXXXX | DNA-2′-OMe); | |
| | | | mG*mC*mA*mG*mG | | | stereorandom PS | |
| WV-1054 | GCCAACAGCCA | 1840 | mGmCmCmAmA*C*A*G*C | 2192 | OOOOXXXXX | 5-7-8 (2′-OMe- | HTT-rs362268 |
| | GCCUGCAGG | | *C*A*G*mCmCmUmGmCm | | XXXOOOOOOO | DNA-2′-OMe); | |
| | | | AmGmG | | | stereorandom PS; | |
| | | | | | | PO in the wings | |
| WV-1055 | GGGCCAACAGC | 1841 | mGmGmGmCmC*A*A*C*A | 2193 | OOOOXXXXX | 5-10-5 (2′-OMe- | HTT-rs362268 |
| | CAGCCUGCA | | *G*C*C*A*G*C*mCmUmG | | XXXXXXOOOO | DNA-2′-OMe); | |
| | | | mCmA | | | stereorandom PS; | |
| | | | | | | PO in the wings | |
| WV-1056 | GGCCAACAGCC | 1842 | mGmGmCmCmA*A*C*A*G | 2194 | OOOOXXXXX | 5-10-5 (2′-OMe- | DNA-2′-OMe); |
| | AGCCUGCAG | | *C*C*A*G*C*C*mUmGmC | | XXXXXXOOOO | DNA-2′-OMe); | |
| | | | mAmG | | | stereorandom PS; | |
| | | | | | | PO in the wings | |
| WV-1057 | GCCAACAGCCA | 1843 | mGmCmCmAmA*C*A*G*C | 2195 | OOOOXXXXX | 5-10-5 (2′-OMe- | HTT-rs362268 |
| | GCCTGCAGG | | *C*A*G*C*C*T*mGmCmA | | XXXXXXOOOO | DNA-2′-OMe); | |
| | | | mGmG | | | stereorandom PS; | |
| | | | | | | PO in the wings | |
| WV-1058 | CCAACAGCCAG | 1844 | mCmCmAmAmC*A*G*C*C | 2196 | OOOOXXXXX | 5-10-5 (2′-OMe- | HTT-rs362268 |
| | CCTGCAGGA | | *A*G*C*C*T*G*mCmAmG | | XXXXXXOOOO | DNA-2′-OMe); | |
| | | | mGmA | | | stereorandom PS; | |
| | | | | | | PO in the wings | |
| WV-1059 | CAACAGCCAGC | 1845 | mCmAmAmCmA*G*C*C*A | 2197 | OOOOXXXXX | 5-10-5 (2′-OMe- | HTT-rs362268 |
| | CTGCAGGAG | | *G*C*C*T*G*C*mAmGmG | | XXXXXXOOOO | DNA-2′-OMe); | |
| | | | mAmG | | | stereorandom PS; | |
| | | | | | | PO in the wings | |
| WV-1060 | AACAGCCAGCC | 1846 | mAmAmCmAmG*C*C*A*G | 2198 | OOOOXXXXX | 5-10-5 (2′-OMe- | HTT-rs362268 |
| | TGCAGGAGG | | *C*C*T*G*C*A*mGmGmA | | XXXXXXOOOO | DNA-2′-OMe); | |
| | | | mGmG | | | stereorandom PS; | |
| | | | | | | PO in the wings: | |
| WV-1061 | GUAGGAGTAGT | 1847 | mG*mU*mA*mG*mG*A*G | 2199 | XXXXXXXXX | 5-10-5 (2′-OMe- | HTT-rs362268 |
| | GAAAGGCCA | | *T*A*G*T*G*A*A*A*mG* | | XXXXXXXXXX | DNA-2′-OMe); | |
| | | | mG*mC*mC*mA | | | stereorandom PS: | |
| | | | | | | +ve Luciferase | |
| | | | | | | control for | |
| | | | | | | psiCHECK2; | |
| | | | | | | WV-975 | |
| | | | | | | analogue | |
| WV-1062 | GUAGGAGTAGT | 1848 | mGmUmAmGmG*A*G*T*A | 2200 | OOOOXXXXX | 5-10-5 (2′-OMe- | HTT-control |
| | GAAAGGCCA | | *G*T*G*A*A*A*mGmGmC | | XXXXXXOOOO | DNA-2′-OMe); | |
| | | | mCmA | | | stereorandom PS; | |
| | | | | | | PO in the wings: | |
| | | | | | | +ve Luciferase | |
| | | | | | | control for | |
| | | | | | | psiCHECK2; | |
| | | | | | | WV-975 | |
| | | | | | | analogue | |
| WV-1063 | GUAGGAGTAGT | 1849 | mG*mU*mA*mG*mG*A*G | 2201 | XXXXXXXXX | 5-15 (2′-OMe- | HTT-control |
| | GAAAGGCCA | | *T*A*G*T*G*A*A*A*G*G | | XXXXXXXXXX | DNA); | |
| | | | *C*C*A | | | stereorandom PS: | |
| | | | | | | +ve Luciferase | |
| | | | | | | control for | |
| | | | | | | psiCHECK2; | |
| | | | | | | WV-975 | |
| | | | | | | analogue | |
| WV-1064 | CUCUUACTGTG | 1850 | mC*mU*mC*mU*mU*A*C* | 2202 | XXXXXXXXX | 5-10-5 (2′-OMe- | HTT-control |
| | CTGTGGACA | | T*G*T*G*C*T*G*T*mG*m | | XXXXXXXXXX | DNA-2′-OMe); | |
| | | | G*mA*mC*mA | | | stereorandom PS: | |
| | | | | | | Negative | |
| | | | | | | Luciferase | |
| | | | | | | control for | |
| | | | | | | psiCHECK2; | |
| | | | | | | ONT-67 | |
| | | | | | | analogue | |
| WV-1065 | CUCUUACTGTG | 1851 | mCmUmCmUmU*A*C*T*G | 2203 | OOOOXXXXX | 5-10-5 (2′-OMe- | HTT-control |
| | CTGTGGACA | | *T*G*C*T*G*T*mGmGmA | | XXXXXXOOO | DNA-2′-OMe); | |
| | | | mCmA | | | stereorandom PS; | |
| | | | | | | PO in the wings: | |
| | | | | | | Negative | |
| | | | | | | Luciferase | |
| | | | | | | control for | |
| | | | | | | psiCHECK2; | |
| | | | | | | ONT-67 | |
| | | | | | | analogue | |
| WV-1066 | CUCUUACTGTG | 1852 | mC*mU*mC*mU*mU*A*C* | 2204 | XXXXXXXXX | 5-15 (2′-OMe- | HTT-control |
| | CTGTGGACA | | T*G*T*G*C*T*G*T*G*G* | | XXXXXXXXXX | DNA); | |
| | | | A*C*A | | | stereorandom PS: | |
| | | | | | | Negative | |
| | | | | | | Luciferase | |
| | | | | | | control for | |
| | | | | | | psiCHECK2; | |
| | | | | | | ONT-67 | |
| | | | | | | analogue | |
| WV-1067 | GGGCACAAGG | 1853 | G*G*G*C*A*C*A*A*G*G* | 2205 | XXXXXXXXX | All DNA | HTT-control |
| | GCACAGACTT | | G*C*d2AP*C*A*G*A*C*T*T | | XXXXXXXXXX | stereorandom; | |
| | | | | | | P13 (2- | |
| | | | | | | aminopurine): | |
| | | | | | | rs362307; WV- | |
| | | | | | | 904 analogue | |
| WV-1068 | GGCACAAGGGC | 1854 | G*G*C*A*C*A*A*G*G*G* | 2206 | XXXXXXXXX | All DNA | rs362307 |
| | ACAGACTTC | | C*d2AP*C*A*G*A*C*T*T*C | | XXXXXXXXXX | stereorandom; | |
| | | | | | | P12 (2- | |
| | | | | | | aminopurine): | |
| | | | | | | rs362307; WV- | |
| | | | | | | 905 analogue | |
| WV-1069 | GCACAAGGGCA | 1855 | G*C*A*C*A*A*G*G*G*C* | 2207 | XXXXXXXXX | All DNA | rs362307 |
| | CAGACTTCC | | d2AP*C*A*G*A*C*T*T*C*C | | XXXXXXXXXX | stereorandom; | |
| | | | | | | P11 (2- | |
| | | | | | | aminopurine): | |
| | | | | | | rs362307; WV- | |
| | | | | | | 906 analogue | |
| WV-1070 | GGGCACAAGG | 1856 | G*G*G*C*A*C*A*A*G*G* | 2208 | XXXXXXXXX | All DNA | rs362307 |
| | GCACAGACTT | | G*C*dDAP*C*A*G*A*C*T | | XXXXXXXXXX | stereorandom; | |
| | | | *T | | | P13 (2; 6- | |
| | | | | | | diaminopurine): | |
| | | | | | | rs362307; WV- | |
| | | | | | | 904 analogue | |
| WV-1071 | GGCACAAGGGC | 1857 | G*G*C*A*C*A*A*G*G*G* | 2209 | XXXXXXXXX | All DNA | rs362307 |
| | ACAGACTTC | | C*dDAP*C*A*G*A*C*T*T | | XXXXXXXXXx | stereorandom; | |
| | | | *C | | | P12 (2; 6- | |
| | | | | | | diaminopurine): | |
| | | | | | | rs362307; WV- | |
| | | | | | | 905 analogue | |
| WV-1072 | GCACAAGGGCA | 1858 | G*C*A*C*A*A*G*G*G*C* | 2210 | XXXXXXXXX | All DNA | rs362307 |
| | CAGACTTCC | | dDAP*C*A*G*A*C*T*T*C | | XXXXXXXXXX | stereorandom; | |
| | | | *C | | | P12 (2;6- | |
| | | | | | | diaminopurine): | |
| | | | | | | rs362307; WV- | |
| | | | | | | 906 analogue | |
| WV-1073 | GAGCCUUUGG | 1859 | rGrArGrCrCrUrUrUrGrGrAr | 2211 | OOOOOOOOO | wtRNA | rs362307 |
| | AAGUCUGCGCC | | ArGrUrCrUrGrCrGrCrCrCrU | | OOOOOOOOO | | |
| | CUUGUGCCCUG | | rUrGrUrGrCrCrCrUrGrCrCrU | | OOOOOOOOO | | |
| | CCU | | | | OOOOOOO | | |
| WV-1074 | GAGCCUUUGG | 1860 | rGrArGrCrCrUrUrUrGrGrAr | 2212 | OOOOOOOOO | muRNA | rs362307 |
| | AAGUCUGUGCC | | ArGrUrCrUrGrUrGrCrCrCrU | | OOOOOOOOO | | |
| | CUUGUGCCCUG | | rUrGrUrGrCrCrCrUrGrCrCrU | | OOOOOOOOO | | |
| | CCU | | | | OOOOOOO | | |
| WV-1075 | CACACGGGCAC | 1861 | rCrArCrArCrGrGrGrCrArCr | 2213 | OOOOOOOOO | Antisense strand: | rs362307 |
| | AGACUUCCAA | | ArGrArCrUrUrCrCrArA | | OOOOOOOOO | Positive control; | |
| | | | | | OO | Curr. Bio. Vol 19 | |
| | | | | | | No 9; 776 | |
| WV-1076 | GGAAGUCUGU | 1862 | rGrGrArArGrUrCrUrGrUrGr | 2214 | OOOOOOOOO | Sense strand: | rs362307 |
| | GCCCGUGUGCC | | CrCrCrGrUrGrUrGrCrC | | OOOOOOOOO | Positive control; | |
| | | | | | OO | Curr. Bio. Vol 19 | |
| | | | | | | No 9; 777: Note: | |
| | | | | | | incorrectly added | |
| | | | | | | as | |
| | | | | | | rGrGrArArGrUr | |
| | | | | | | CrUrGrUrGrCrC | |
| | | | | | | rCrGrUrGrUrUr | |
| | | | | | | CrC (SEQ ID | |
| | | | | | | NO: 2417) in | |
| | | | | | | earlier versions | |
| | | | | | | of databse | |
| WV-1077 | AUUAAUAAATT | 1863 | mA*SmU*SmU*SmA*SmA* | 2215 | SSSSSSSSSSSS | 6-10-4 (2′-OMe- | HTT rs7685686 |
| | GTCATCACC | | SmU*SA*SA*SA*ST*ST*S | | SRSSSSS | DNA-2′-OMe) | |
| | | | G*ST*SC*RA*ST*SmC*Sm | | | Gapmer: | |
| | | | A*SmC*SmC | | | Analogue of | |
| | | | | | | WV-451 | |
| WV-1078 | AUUAAUAAATT | 1864 | mA*RmU*RmU*RmA*RmA | 2216 | RRRRRSSSSSS | 6-10-4 (2′-OMe- | HTT rs7685686 |
| | GTCATCACC | | *RmU*SA*SA*SA*ST*ST* | | SSRSSRRR | DNA-2′-OMe) | |
| | | | SG*ST*SC*RA*ST*SmC*R | | | Gapmer: | |
| | | | mA*RmC*RmC | | | Analogue of | |
| | | | | | | WV-451 | |
| WV-1079 | AUUAAUAAATT | 1865 | mA*SmU*SmU*SmA*SmA* | 2217 | SSSSSSSSSSSS | 8-12 (2′-OMe- | HTT rs7685686 |
| | GTCATCACC | | SmU*SmA*SmA*SA*ST*ST | | SRSSSSS | DNA) hemimer: | |
| | | | *SG*ST*SC*RA*ST*SC*SA | | | Analogue of | |
| | | | *SC*SC | | | WV-451 | |
| WV-1080 | AUUAAUAAATT | 1866 | mA*RmU*RmU*RmA*RmA | 2218 | RRRRRRRSSSS | 8-12 (2′-OMe- | HTT rs7685686 |
| | GTCATCACC | | *RmU*RmA*RmA*SA*ST* | | SSRSSSSS | DNA) hemimer: | |
| | | | ST*SG*ST*SC*RA*ST*SC* | | | Analogue of | |
| | | | SA*SC*SC | | | WV-451 | |
| WV-1081 | AUUAAUAAATT | 1867 | mAmUmUmAmAmUmAmA | 2219 | OOOOOOOSSS | 8-12 (2′-OMe- | HTT rs7685686 |
| | GTCATCACC | | *SA*ST*ST*SG*ST*SC*RA | | SSSRSSSSS | DNA) hemimer; | |
| | | | *ST*SC*SA*SC*SC | | | PO wing: | |
| | | | | | | Analogue of | |
| | | | | | | WV-451 | |
| WV-1082 | AUUAAUAAATT | 1868 | mAmUmUmAmAmU*SA*S | 2220 | OOOOOSSSSSS | 6-10-4 (2′-OMe- | HTT rs7685686 |
| | GTCATCACC | | A*SA*ST*ST*SG*ST*SC*R | | SSRSSOOO | DNA-2′-OMe); | |
| | | | A*ST*SmCmAmCmC | | | PO wings: | |
| | | | | | | Analogue of | |
| | | | | | | WV-451 | |
| WV-1083 | AUUAAUAAATT | 1869 | mA*SmUmUmAmAmU*SA | 2221 | SOOOOSSSSSS | 6-10-4 (2′-OMe- | HTT rs7685686 |
| | GTCATCACC | | *SA*SA*ST*ST*SG*ST*SC | | SSRSSOOS | DNA-2′-OMe) | |
| | | | *RA*ST*SmCmAmC*SmC | | | Gapmer: | |
| | | | | | | Analogue of | |
| | | | | | | WV-451 | |
| WV-1084 | AUUAAUAAATT | 1870 | mA*RmUmUmAmAmU*SA | 2222 | ROOOOSSSSSS | 6-10-4 (2′-OMe- | HTT rs7685686 |
| | GTCATCACC | | *SA*SA*ST*ST*SG*ST*SC | | SSRSSOOR | DNA-2′-OMe) | |
| | | | *RA*ST*SmCmAmC*RmC | | | Gapmer: | |
| | | | | | | Analogue of | |
| | | | | | | WV-451 | |
| WV-1085 | GGCACAAGGGC | 1871 | mG*SmG*SmC*SmA*SmC* | 2223 | SSSSSSSSSSSS | 5-10-5 (2′-OMe- | HTT rs362307 |
| | ACAGACUUC | | SA*SA*SG*SG*SG*SC*SA | | RSSSSSS | DNA-2′-OMe) | |
| | | | *SC*RA*SG*SmA*SmC*Sm | | | Gapmer: | |
| | | | U*SmU*SmC | | | Analogue of | |
| | | | | | | WV-905 and | |
| | | | | | | WV-937 | |
| WV-1086 | GGCACAAGGGC | 1872 | mG*RmG*RmC*RmA*RmC | 2224 | RRRRSSSSSSS | 5-10-5 (2′-OMe- | HTT rs362307 |
| | ACAGACUUC | | *SA*SA*SG*SG*SG*SC*S | | SRSSRRRR | DNA-2′-OMe) | |
| | | | A*SC*RA*SG*SmA*RmC* | | | Gapmer: | |
| | | | RmU*RmU*RmC | | | Analogue of | |
| | | | | | | WV-905 and | |
| | | | | | | WV-937 | |
| WV-1087 | GGCACAAGGGC | 1873 | mGmGmCmAmC*SA*SA*S | 2225 | OOOOSSSSSSS | 5-10-5 (2′-OMe- | HTT rs362307 |
| | ACAGACUUC | | G*SG*SG*SC*SA*SC*RA* | | SRSSOOOO | DNA-2′-OMe); | |
| | | | SG*SmAmCmUmUmC | | | PO wings: | |
| | | | | | | Analogue of | |
| | | | | | | WV-905 and | |
| | | | | | | WV-937 | |
| WV-1088 | GGCACAAGGGC | 1874 | mG*SmG*SmC*SmA*SmC* | 2226 | SSSSSSSSSSSS | 8-12 (2′-OMe- | HTT rs362307 |
| | ACAGACTTC | | SmA*SmA*SmG*SG*SG*S | | RSSSSSS | DNA) hemimer: | |
| | | | C*SA*SC*RA*SG*SA*SC* | | | Analogue of | |
| | | | ST*ST*SC | | | WV-905 and | |
| | | | | | | WV-937 | |
| WV-1089 | GGCACAAGGGC | 1875 | mG*RmG*RmC*RmA*RmC | 2227 | RRRRRRRSSSS | 8-12 (2′-OMe- | HTT rs362307 |
| | ACAGACTTC | | *RmA*RmA*RmG*SG*SG* | | SRSSSSSS | DNA) hemimer: | |
| | | | SC*SA*SC*RA*SG*SA*SC | | | Analogue of | |
| | | | *ST*ST*SC | | | WV-905 and | |
| | | | | | | WV-937 | |
| WV-1090 | GGCACAAGGGC | 1876 | mGmGmCmAmCmAmAmG | 2228 | OOOOOOOSSS | 8-12 (2′-OMe- | HTT rs362307 |
| | ACAGACTTC | | *SG*SG*SC*SA*SC*RA*S | | SSRSSSSSS | DNA) hemimer; | |
| | | | G*SA*SC*ST*ST*SC | | | PO wing: | |
| | | | | | | Analogue of | |
| | | | | | | WV-905 and | |
| | | | | | | WV-937 | |
| WV-1091 | GGCACAAGGGC | 1877 | mG*RmGmCmAmC*SA*SA | 2229 | ROOOSSSSSSS | 8-12 (2′-OMe- | HTT rs362307 |
| | ACAGACUUC | | *SG*SG*SG*SC*SA*SC*R | | SRSSOOOR | DNA) gapmer | |
| | | | A*SG*SmAmCmUmU*RmC | | | PO wing: | |
| | | | | | | Analogue of | |
| | | | | | | WV-905 and | |
| | | | | | | WV-937: | |
| | | | | | | incorrectly added | |
| | | | | | | as | |
| | | | | | | gsSgcacsSdAsSd | |
| | | | | | | AsSdGsSdGsSd | |
| | | | | | | GsSdCsSdAsSd | |
| | | | | | | CsRdAsSdGsSac | |
| | | | | | | uusSc (SEQ ID | |
| | | | | | | NO: 2418) in | |
| | | | | | | earlier version of | |
| | | | | | | database | |
| WV-1092 | GGCACAAGGGC | 1878 | mG*SmGmCmAmC*SA*SA | 2230 | SOOOSSSSSSS | 8-12 (2′-OMe- | HTT rs362307 |
| | ACAGACUUC | | *SG*SG*SG*SC*SA*SC*R | | SRSSOOOS | DNA) gapmer | |
| | | | A*SG*SmAmCmUmU*SmC | | | PO wing: | |
| | | | | | | Analogue of | |
| | | | | | | WV-905 and | |
| | | | | | | WV-937 | |
| WV-1183 | GCAGGGCACAA | 1879 | G*C*A*G*G*G*C*A*C*A* | 2231 | XXXXXXXXX | Phosphorothioate | Huntington |
| | GGGCACAGA | | A*G*G*G*C*A*C*A*G*A | | XXXXXXXXXX | DNA; | rs362307 |
| | | | | | | Stereorandom | |
| WV-1184 | GCAGGGCACAA | 1880 | mG*mC*mA*mG*mG*G*C | 2232 | XXXXXXXXX | 5-15 (2′-OMe- | Huntington |
| | GGGCACAGA | | *A*C*A*A*G*G*G*C*A*C | | XXXXXXXXXX | DNA) Hemimer | rs362307 |
| | | | *A*G*A | | | | |
| WV-1185 | GCAGGGCACAA | 1881 | mGmCmAmGmG*G*C*A*C | 2233 | OOOOXXXXX | 5-15 (2′-OMe- | Huntington |
| | GGGCACAGA | | *A*A*G*G*G*C*A*C*A*G | | XXXXXXXXXX | DNA) Hemimer; | rs362307 |
| | | | *A | | | PO wing | |
| WV-1186 | GCAGGGCACAA | 1882 | mG*mC*mA*mG*mG*mG* | 2234 | XXXXXXXXX | 7-13 (2′-OMe- | Huntington |
| | GGGCACAGA | | mC*A*C*A*A*G*G*G*C* | | XXXXXXXXXX | DNA) Hemimer | rs362307 |
| | | | A*C*A*G*A | | | | |
| WV-1187 | GCAGGGCACAA | 1883 | mGmCmAmGmGmGmC*A* | 2235 | OOOOOOXXX | 7-13 (2′-OMe- | Huntington |
| | GGGCACAGA | | C*A*A*G*G*G*C*A*C*A* | | XXXXXXXXXX | DNA) Hemimer; | rs362307 |
| | | | G*A | | | PO wing | |
| WV-1188 | CAGGGCACAAG | 1884 | C*A*G*G*G*C*A*C*A*A* | 2236 | XXXXXXXXX | Phosphorothioate | Huntington |
| | GGCACAGAC | | G*G*G*C*A*C*A*G*A*C | | XXXXXXXXXX | DNA; | rs362307 |
| | | | | | | Stereorandom | |
| WV-1189 | CAGGGCACAAG | 1885 | mC*mA*mG*mG*mG*C*A | 2237 | XXXXXXXXX | 5-15 (2′-OMe- | Huntington |
| | GGCACAGAC | | *C*A*A*G*G*G*C*A*C*A | | XXXXXXXXXX | DNA) Hemimer | rs362307 |
| | | | *G*A*C | | | | |
| WV-1190 | CAGGGCACAAG | 1886 | mCmAmGmGmG*C*A*C*A | 2238 | OOOOXXXXX | 5-15 (2′-OMe- | Huntington |
| | GGCACAGAC | | *A*G*G*G*C*A*C*A*G*A | | XXXXXXXXXX | DNA) Hemimer; | rs362307 |
| | | | *C | | | PO wing | |
| WV-1191 | CAGGGCACAAG | 1887 | mC*mA*mG*mG*mG*mC* | 2239 | XXXXXXXXX | 7-13 (2′-OMe- | Huntington |
| | GGCACAGAC | | mA*C*A*A*G*G*G*C*A* | | XXXXXXXXXX | DNA) Hemimer | rs362307 |
| | | | mC*mA*mG*mA*mC | | | | |
| WV-1192 | CAGGGCACAAG | 1888 | mCmAmGmGmGmCmA*C* | 2240 | OOOOOOXXX | 7-13 (2′-OMe- | Huntington |
| | GGCACAGAC | | A*A*G*G*G*C*A*mCmAm | | XXXXXXOOOO | DNA) Hemimer; | rs362307 |
| | | | GmAmC | | | PO wing | |
| WV-1193 | AGGGCACAAG | 1889 | A*G*G*G*C*A*C*A*A*G* | 2241 | XXXXXXXXX | Phosphorothioate | Huntington |
| | GGCACAGACT | | G*G*C*A*C*A*G*A*C*T | | XXXXXXXXXX | DNA; | rs362307 |
| | | | | | | Stereorandom | |
| WV-1194 | AGGGCACAAG | 1890 | mA*mG*mG*mG*mC*A*C | 2242 | XXXXXXXXX | 5-15 (2′-OMe- | Huntington |
| | GGCACAGACT | | *A*A*G*G*G*C*A*C*A*G | | XXXXXXXXXX | DNA) Hemimer | rs362307 |
| | | | *A*C*T | | | | |
| WV-1195 | AGGGCACAAG | 1891 | mAmGmGmGmC*A*C*A*A | 2243 | OOOOXXXXX | 5-15 (2′-OMe- | Huntington |
| | GGCACAGACT | | *G*G*G*C*A*C*A*G*A*C | | XXXXXXXXXX | DNA) Hemimer; | rs362307 |
| | | | *T | | | PO wing | |
| WV-1196 | AGGGCACAAG | 1892 | mA*mG*mG*mG*mC*mA* | 2244 | XXXXXXXXX | 7-12-1 (2′-OMe- | Huntington |
| | GGCACAGACU | | mC*A*A*G*G*G*C*A*C* | | XXXXXXXXXX | DNA-2′-DNA) | rs362307 |
| | | | A*G*A*C*mU | | | Gapmer | |
| WV-1197 | AGGGCACAAG | 1893 | mAmGmGmGmCmAmC*A* | 2245 | OOOOOOXXX | 7-12-1 (2′-OMe- | Huntington |
| | GGCACAGACU | | A*G*G*G*C*A*C*A*G*A* | | XXXXXXXXXX | DNA-2′-DNA) | rs362307 |
| | | | C*mU | | | Gapmer; PO | |
| | | | | | | wings | |
| WV-1198 | AAGGGCACAG | 1894 | A*A*G*G*G*C*A*C*A*G* | 2246 | XXXXXXXXX | Phosphorothioate | Huntington |
| | ACTTCCAAAG | | A*C*T*T*C*C*A*A*A*G | | XXXXXXXXXX | DNA | rs362307 |
| | | | | | | Stereorandom | |
| WV-1199 | AAGGGCACAG | 1895 | mA*mA*mG*mG*mG*C*A | 2247 | XXXXXXXXX | 5-15 (2′-OMe- | Huntington |
| | ACTTCCAAAG | | *C*A*G*A*C*T*T*C*C*A* | | XXXXXXXXXX | DNA) Hemimer | rs362307 |
| | | | A*A*G | | | | |
| WV-1200 | AAGGGCACAG | 1896 | mAmAmGmGmG*C*A*C*A | 2248 | OOOOXXXXX | 5-15 (2′-OMe- | Huntington |
| | ACTTCCAAAG | | *G*A*C*T*T*C*C*A*A*A | | XXXXXXXXXX | DNA) Hemimer; | rs362307 |
| | | | *G | | | PO wing | |
| WV-1201 | AAGGGCACAG | 1897 | mA*mA*mG*mG*mG*C*A | 2249 | XXXXXXXXX | 5-10-5 (2′-OMe- | Huntington |
| | ACTTCCAAAG | | *C*A*G*A*C*T*T*C*mC* | | XXXXXXXXXX | DNA-2′-DNA) | rs362307 |
| | | | mA*mA*mA*mG | | | Gapmer | |
| WV-1202 | AAGGGCACAG | 1898 | mAmAmGmGmG*C*A*C*A | 2250 | OOOOXXXXX | 5-10-5 (2′-OMe- | Huntington |
| | ACTTCCAAAG | | *G*A*C*T*T*C*mCmAmA | | XXXXXXOOOO | DNA-2′-DNA) | rs362307 |
| | | | mAmG | | | Gapmer; PO | |
| | | | | | | wings | |
| WV-1203 | AAGGGCACAG | 1899 | mA*mA*mG*mG*G*C*A*C | 2251 | XXXXXXXXX | 4-10-6 (2′-OMe- | Huntington |
| | ACTTCCAAAG | | *A*G*A*C*T*T*mC*mC*m | | XXXXXXXXXX | DNA-2′-DNA) | rs362307 |
| | | | A*mA*mA*mG | | | Gapmer | |
| WV-1204 | AAGGGCACAG | 1900 | mAmAmGmGG*C*A*C*A* | 2252 | OOOOXXXXX | 4-10-6 (2′-OMe- | Huntington |
| | ACTTCCAAAG | | G*A*C*T*T*mCmCmAmA | | XXXXXOOOOO | DNA-2′-DNA) | rs362307 |
| | | | mAmG | | | Gapmer; PO | |
| | | | | | | wings | |
| WV-1205 | AGGGCACAGAC | 1901 | A*G*G*G*C*A*C*A*G*A* | 2253 | XXXXXXXXX | Phosphorothioate | Huntington |
| | TTCCAAAGG | | C*T*T*C*C*A*A*A*G*G | | XXXXXXXXXX | DNA; | rs362307 |
| | | | | | | Stereorandom | |
| WV-1206 | AGGGCACAGAC | 1902 | mA*mG*mG*mG*mC*A*C | 2254 | XXXXXXXXX | 5-15 (2′-OMe- | Huntington |
| | TTCCAAAGG | | *A*G*A*C*T*T*C*C*A*A | | XXXXXXXXXX | DNA) Hemimer | rs362307 |
| | | | *A*G*G | | | | |
| WV-1207 | AGGGCACAGAC | 1903 | mAmGmGmGmC*A*C*A*G | 2255 | OOOOXXXXX | 5-15 (2′-OMe- | Huntington |
| | TTCCAAAGG | | *A*C*T*T*C*C*A*A*A*G | | XXXXXXXXXX | DNA) Hemimer; | rs362307 |
| | | | *G | | | PO wing | |
| WV-1208 | AGGGCACAGAC | 1904 | mA*mG*mG*mG*mC*A*C | 2256 | XXXXXXXXX | 5-10-5 (2′-OMe- | Huntington |
| | TTCCAAAGG | | *A*G*A*C*T*T*C*C*mA* | | XXXXXXXXXX | DNA-2′-DNA) | rs362307 |
| | | | mA*mA*mG*mG | | | Gapmer | |
| WV-1209 | AGGGCACAGAC | 1905 | mAmGmGmGmC*A*C*A*G | 2257 | OOOOXXXXX | 5-10-5 (2′-OMe- | Huntington |
| | TTCCAAAGG | | *A*C*T*T*C*C*mAmAmA | | XXXXXXOOOO | DNA-2′-DNA) | rs362307 |
| | | | mGmG | | | Gapmer; PO | |
| | | | | | | wings | |
| WV-1210 | AGGGCACAGAC | 1906 | mA*mG*mG*mG*C*A*C*A | 2258 | XXXXXXXXX | 4-10-6 (2′-OMe- | Huntington |
| | TTCCAAAGG | | *G*A*C*T*T*C*mC*mA*m | | XXXXXXXXXX | DNA-2′-DNA) | rs362307 |
| | | | A*mA*mG*mG | | | Gapmer | |
| WV-1211 | AGGGCACAGAC | 1907 | mAmGmGmG*C*A*C*A*G | 2259 | OOOXXXXXX | 4-10-6 (2′-OMe- | Huntington |
| | TTCCAAAGG | | *A*C*T*T*C*mCmAmAmA | | XXXXXOOOOO | DNA-2′-DNA) | rs362307 |
| | | | mGmG | | | Gapmer; PO | |
| | | | | | | wings | |
| WV-1212 | GGGCACAGACT | 1908 | G*G*G*C*A*C*A*G*A*C* | 2260 | XXXXXXXXX | Phosphorothioate | Huntington |
| | TCCAAAGGC | | T*T*C*C*A*A*A*G*G*C | | XXXXXXXXXX | DNA; | rs362307 |
| | | | | | | Stereorandom | |
| WV-1213 | GGGCACAGACT | 1909 | mG*mG*mG*mC*mA*C*A | 2261 | XXXXXXXXX | 4-16 (2′-OMe- | Huntington |
| | TCCAAAGGC | | *G*A*C*T*T*C*C*A*A*A | | XXXXXXXXXX | DNA) Hemimer | rs362307 |
| | | | *G*G*C | | | | |
| WV-1214 | GGGCACAGACT | 1910 | mGmGmGmCmA*C*A*G*A | 2262 | OOOOXXXXX | 4-16 (2′-OMe- | Huntington |
| | TCCAAAGGC | | *C*T*T*C*C*A*A*A*G*G | | XXXXXXXXXX | DNA) Hemimer; | rs362307 |
| | | | *C | | | PO wing | |
| WV-1215 | GGGCACAGACT | 1911 | mG*mG*mG*mC*mA*C*A | 2263 | XXXXXXXXX | 4-10-6 (2′-OMe- | Huntington |
| | TCCAAAGGC | | *G*A*C*T*T*C*C*A*mA* | | XXXXXXXXXX | DNA-2′-DNA) | rs362307 |
| | | | mA*mG*mG*mC | | | Gapmer | |
| WV-1216 | GGGCACAGACT | 1912 | mGmGmGmCmA*C*A*G*A | 2264 | OOOOXXXXX | 4-10-6 (2′-OMe- | Huntington |
| | TCCAAAGGC | | *C*T*T*C*C*A*mAmAmG | | XXXXXXOOOO | DNA-2′-DNA) | rs362307 |
| | | | mGmC | | | Gapmer; PO | |
| | | | | | | wings | |
| WV-1234 | GGCACAAGGGC | 1913 | mG*mG*mC*mA*mC*A*A | 2265 | XXXXXXXXX | 5-10-5 (2′-OMe- | HTT-rs362307 |
| | ACAGACUTC | | *G*G*G*C*A*C*A*G*mA* | | XXXXXXXXXX | DNA-2′-OMe) | |
| | | | mC*mU*BrdU*mC | | | Gapmer; One Br- | |
| | | | | | | dU | |
| WV-1235 | GGCACAAGGGC | 1914 | mG*mG*mC*mA*mC*A*A | 2266 | XXXXXXXXX | 5-10-5 (2′-OMe- | HTT-rs362307 |
| | ACAGACTTC | | *G*G*G*C*A*C*A*G*mA* | | XXXXXXXXXX | DNA-2′-OMe) | |
| | | | mC*BrdU*BrdU*mC | | | Gapmer; two Br- | |
| | | | | | | dU | |
| WV-1497 | GGCACAAGGGC | 1915 | mG*mGmCmAmC*A*A*G* | 2267 | XOOOXXXXX | stereo random | HTT rs362307 |
| | ACAGACUUC | | G*G*C*A*C*A*G*mAmCm | | XXXXXXOOOX | version of WV- | |
| | | | UmU*mC | | | 1092 | |
| WV-1508 | AUUAAUAAATT | 1916 | A*SmUmUmAmAmU*SA*S | 2268 | SOOOOSSSSSS | 1-5-10-3-1 | HTT rs7685686 |
| | GTCATCACC | | A*SA*ST*ST*SG*ST*SC*R | | SSRSSOOS | (DNA/2′-OMe) | |
| | | | A*ST*SmCmAmC*SC | | | Gapmer:: | |
| | | | | | | Analogue of | |
| | | | | | | WV-1083 | |
| WV-1509 | AUUAAUAAATT | 1917 | A*mUmUmAmAmU*A*A* | 2269 | XOOOOXXXX | 1-5-10-3-1 | HTT rs7685686 |
| | GTCATCACC | | A*T*T*G*T*C*A*T*mCmA | | XXXXXXXOOX | (DNA/2′-OMe) | |
| | | | mC*C | | | Gapmer; 1st and | |
| | | | | | | last PS:: | |
| | | | | | | Analogue of | |
| | | | | | | WV-1083 | |
| WV-1510 | GGCACAAGGGC | 1918 | G*SmGmCmAmC*SA*SA*S | 2270 | SOOOSSSSSSS | 1-4-10-4-1 | HTT rs362307 |
| | ACAGACUUC | | G*SG*SG*SC*SA*SC*RA* | | SRSSOOOS | (DNA/2′-OMe) | |
| | | | SG*SmAmCmUmU*SC | | | gapmer:: | |
| | | | | | | Analogue of | |
| | | | | | | WV-1092 | |
| WV-1511 | GGCACAAGGGC | 1919 | G*mGmCmAmC*A*A*G*G | 2271 | XOOOXXXXX | 1-4-10-4-1 | HTT rs362307 |
| | ACAGACUUC | | *G*C*A*C*A*G*mAmCmU | | XXXXXXOOOX | (DNA/2′-OMe) | |
| | | | mU*C | | | gapmer; 1st and | |
| | | | | | | last PS:: | |
| | | | | | | Analogue of | |
| | | | | | | WV-1092 | |
| WV-1654 | GGCACAAGGGC | 1920 | Geo*Geo*m5Ceo*Aeo*m5Ce | 2272 | XXXXXXXXX | 5-10-5; 2′- | HTT rs362307 |
| | ACAGACTTC | | o*A*A*G*G*G*C*A*C*A* | | XXXXXXXXXX | OMOE gapmer; | |
| | | | G*Aeo*m5Ceo*Teo*Teo*m5 | | | All PS | |
| | | | Ceo | | | | |
| WV-1655 | GGCACAAGGGC | 1921 | Geo*Geom5CeoAeom5Ceo* | 2273 | XOOOXXXXX | 5-10-5; 2′- | HTT rs362307 |
| | ACAGACTTC | | A*A*G*G*G*C*A*C*A*G* | | XXXXXXOOOX | OMOE gapmer; | |
| | | | Aeom5CeoTeoTeo*m5Ceo | | | 1st and last PS in | |
| | | | | | | the wing; rest of | |
| | | | | | | the wing is PO | |
| WV-1656 | CTCAGTAACAT | 1922 | m5Ceo*Teo*m5Ceo*Aeo*Ge | 2274 | XXXXXXXXX | 5-10-5; 2′- | Huntington |
| | TGACACCAC | | o*T*A*A*C*A*T*T*G*A*C | | XXXXXXXXXX | OMOE gapmer; | |
| | | | *Aeo*m5Ceo*m5Ceo*Aeo* | | | All PS | |
| | | | m5Ceo | | | | |
| WV-1657 | CUCAGTAACAT | 1923 | mC*mU*mC*mA*mG*T*A* | 2275 | XXXXXXXXX | 5-10-5; 2′-OMe | Huntington |
| | TGACACCAC | | A*C*A*T*T*G*A*C*mA*m | | XXXXXXXXXX | gapmer; All PS | |
| | | | C*mC*mA*mC | | | | |
| WV-1788 | GGCACAAGGGC | 1924 | mG*mGmCmAmC*A*A*G* | 2276 | XOOOXXXXX | 5/10/5 2′Ome | HTT |
| | ACAGACUTC | | G*G*C*A*C*A*G*mAmCm | | XXXXXXOOXX | Gapmer BrdU | |
| | | | U*BrdU*mC | | | PO wings | |
| WV-1789 | CTCAGTAACAT | 1925 | mC*BrdU*mC*mA*mG*T* | 2277 | XXXXXXXXX | 5/10/5 2′Ome | HTT |
| | TGACACCAC | | A*A*C*A*T*T*G*A*C*mA | | XXXXXXXXX | Gapmer BrdU | |
| | | | *mC*mC*mA*mC | | | | |
| WV-1790 | CTCAGTAACAT | 1926 | mC*BrdU*mCmAmG*T*A* | 2278 | XXOOXXXXX | 5/10/5 2′Ome | HTT |
| | TGACACCAC | | A*C*A*T*T*G*A*C*mAm | | XXXXXXOOOX | Gapmer BrdU | |
| | | | CmCmA*mC | | | PO wings | |
| WV-1799 | GAAGUCUGUG | 1927 | rGrArArGrUrCrUrGrUrGrCr | 2279 | OOOOOOOOO | RNA | HTT |
| | CCCUUGUGCC | | CrCrUrUrGrUrGrCrC | | OOOOOOOOOO | complementary | |
| | | | | | | to WV1092 | |
| WV-2022 | GGCACAAGGGC | 1928 | mG*SmGmCmAmC*SA*SA | 2280 | SOOOSSSSSSS | BrdU version of | HTT rs362307 |
| | ACAGACUTC | | *SG*SG*SG*SC*SA*SC*R | | SRSSOOSS | WV-1092 | |
| | | | A*SG*SmAmCmU*SBrdU* | | | | |
| | | | SmC | | | | |
| WV-2023 | TGTCATCACCA | 1929 | T*G*T*C*A*T*C*A*C*C* | 2281 | XXXXXXXXX | 15-5 hemimer | rs7685686 |
| | GAAAAAGUC | | A*G*A*A*A*mA*mA*mG* | | XXXXXXXXXX | full PS | (A/G) |
| | | | mU*mC | | | | |
| WV-2024 | UTGTCATCACC | 1930 | mU*T*G*T*C*A*T*C*A*C | 2282 | XXXXXXXXX | 1-14-5 gapmer | rs7685686 |
| | AGAAAAAGU | | *C*A*G*A*A*mA*mA*mA | | XXXXXXXXXX | full PS | (A/G) |
| | | | *mG*mU | | | | |
| WV-2025 | TTGTCATCACC | 1931 | T*T*G*T*C*A*T*C*A*C*C | 2283 | XXXXXXXXX | 15-5 hemimer | rs7685686 |
| | AGAAAAAGU | | *A*G*A*A*mA*mA*mA*m | | XXXXXXXXXX | full PS | (A/G) |
| | | | G*mU | | | | |
| WV-2026 | AUTGTCATCAC | 1932 | mA*mU*T*G*T*C*A*T*C* | 2284 | XXXXXXXXX | 2-13-5 gapmer | rs7685686 |
| | CAGAAAAAG | | A*C*C*A*G*A*mA*mA*m | | XXXXXXXXXX | full PS | (A/G) |
| | | | A*mA*mG | | | | |
| WV-2027 | ATTGTCATCAC | 1933 | mA*T*T*G*T*C*A*T*C*A | 2285 | XXXXXXXXX | 1-14-5 gapmer | rs7685686 |
| | CAGAAAAAG | | *C*C*A*G*A*mA*mA*mA | | XXXXXXXXXX | full PS | (A/G) |
| | | | *mA*mG | | | | |
| WV-2028 | AAUTGTCATCA | 1934 | mA*mA*mU*T*G*T*C*A* | 2286 | XXXXXXXXX | 3-12-5 gapmer | rs7685686 |
| | CCAGAAAAA | | T*C*A*C*C*A*G*mA*mA* | | XXXXXXXXXX | full PS | (A/G) |
| | | | mA*mA*mA | | | | |
| WV-2029 | AATTGTCATCA | 1935 | mA*mA*T*T*G*T*C*A*T* | 2287 | XXXXXXXXX | 2-13-5 gapmer | rs7685686 |
| | CCAGAAAAA | | C*A*C*C*A*G*mA*mA*m | | XXXXXXXXX | full PS | (A/G) |
| | | | A*mA*mA | | | | |
| WV-2030 | AAATTGTCATC | 1936 | mA*mA*mA*T*T*G*T*C* | 2288 | XXXXXXXXX | 3-12-5 gapmer | rs7685686 |
| | ACCAGAAAA | | A*T*C*A*C*C*A*mG*mA* | | XXXXXXXXXX | full PS | (A/G) |
| | | | mA*mA*mA | | | | |
| WV-2031 | AAAUTGTCATC | 1937 | mA*mA*mA*mU*T*G*T*C | 2289 | XXXXXXXXX | 4-11-5 gapmer | rs7685686 |
| | ACCAGAAAA | | *A*T*C*A*C*C*A*mG*mA | | XXXXXXXXXX | full PS | (A/G) |
| | | | *mA*mA*mA | | | | |
| WV-2032 | UAAAUTGTCAT | 1938 | mU*mA*mA*mA*mU*T*G* | 2290 | XXXXXXXXX | 5-11-4 gapmer | rs7685686 |
| | CACCAGAAA | | T*C*A*T*C*A*C*C*A*mG | | XXXXXXXXXX | full PS | (A/G) |
| | | | *mA*mA*mA | | | | |
| WV-2033 | UAAAUTGTCAT | 1939 | mU*mA*mA*mA*mU*T*G* | 2291 | XXXXXXXXX | 5-10-5 gapmer | rs7685686 |
| | CACCAGAAA | | T*C*A*T*C*A*C*C*mA*m | | XXXXXXXXXX | full PS | (A/G) |
| | | | G*mA*mA*mA | | | | |
| WV-2034 | AUAAATTGTCA | 1940 | mA*mU*mA*mA*mA*T*T* | 2292 | XXXXXXXXX | 5-11-4 gapmer | rs7685686 |
| | TCACCAGAA | | G*T*C*A*T*C*A*C*C*mA | | XXXXXXXXXX | full PS | (A/G) |
| | | | *mG*mA*mA | | | | |
| WV-2035 | AUAAATTGTCA | 1941 | mA*mU*mA*mA*mA*T*T* | 2293 | XXXXXXXXX | 5-10-5 gapmer | rs7685686 |
| | TCACCAGAA | | G*T*C*A*T*C*A*C*mC*m | | XXXXXXXXXX | full PS | (A/G) |
| | | | A*mG*mA*mA | | | | |
| WV-2036 | AAUAAATTGTC | 1942 | mA*mA*mU*mA*mA*A*T* | 2294 | XXXXXXXXX | 5-12-3 gapmer | rs7685686 |
| | ATCACCAGA | | T*G*T*C*A*T*C*A*C*C* | | XXXXXXXXXX | full PS | (A/G) |
| | | | mA*mG*mA | | | | |
| WV-2037 | AAUAAATTGTC | 1943 | mA*mA*mU*mA*mA*A*T* | 2295 | XXXXXXXXX | 5-11-4 gapmer | rs7685686 |
| | ATCACCAGA | | T*G*T*C*A*T*C*A*C*mC | | XXXXXXXXXX | full PS | (A/G) |
| | | | *mA*mG*mA | | | | |
| WV-2038 | AAUAAATTGTC | 1944 | mA*mA*mU*mA*mA*A*T* | 2296 | XXXXXXXXX | 5-10-5 gapmer | rs7685686 |
| | ATCACCAGA | | T*G*T*C*A*T*C*A*mC*m | | XXXXXXXXXX | full PS | (A/G) |
| | | | C*mA*mG*mA | | | | |
| WV-2039 | UAAUAAATTGT | 1945 | mU*mA*mA*mU*mA*A*A | 2297 | XXXXXXXXX | 5-13-2 gapmer | rs7685686 |
| | CATCACCAG | | *T*T*G*T*C*A*T*C*A*C* | | XXXXXXXXXX | full PS | (A/G) |
| | | | C*mA*mG | | | | |
| WV-2040 | UAAUAAATTGT | 1946 | mU*mA*mA*mU*mA*A*A | 2298 | XXXXXXXXX | 5-12-3 gapmer | rs7685686 |
| | CATCACCAG | | *T*T*G*T*C*A*T*C*A*C* | | XXXXXXXXXX | full PS | (A/G) |
| | | | mC*mA*mG | | | | |
| WV-2041 | UAAUAAATTGT | 1947 | mU*mA*mA*mU*mA*A*A | 2299 | XXXXXXXXX | 5-11-4 gapmer | rs7685686 |
| | CATCACCAG | | *T*T*G*T*C*A*T*C*A*m | | XXXXXXXXXX | full PS | (A/G) |
| | | | C*mC*mA*mG | | | | |
| WV-2042 | UAAUAAATTGT | 1948 | mU*mA*mA*mU*mA*A*A | 2300 | XXXXXXXXX | 5-10-5 gapmer | rs7685686 |
| | CATCACCAG | | *T*T*G*T*C*A*T*C*mA* | | XXXXXXXXXX | full PS | (A/G) |
| | | | mC*mC*mA*mG | | | | |
| WV-2043 | UUAAUAAATTG | 1949 | mU*mU*mA*mA*mU*A*A | 2301 | XXXXXXXXX | 5-14-1 gapmer | rs7685686 |
| | TCATCACCA | | *A*T*T*G*T*C*A*T*C*A* | | XXXXXXXXXX | full PS | (A/G) |
| | | | C*C*mA | | | | |
| WV-2044 | UUAAUAAATTG | 1950 | mU*mU*mA*mA*mU*A*A | 2302 | XXXXXXXXX | 5-13-2 gapmer | rs7685686 |
| | TCATCACCA | | *A*T*T*G*T*C*A*T*C*A* | | XXXXXXXXXX | full PS | (A/G) |
| | | | C*mC*mA | | | | |
| WV-2045 | UUAAUAAATTG | 1951 | mU*mU*mA*mA*mU*A*A | 2303 | XXXXXXXXX | 5-12-3 gapmer | rs7685686 |
| | TCATCACCA | | *A*T*T*G*T*C*A*T*C*A* | | XXXXXXXXXX | full PS | (A/G) |
| | | | mC*mC*mA | | | | |
| WV-2046 | UUAAUAAATTG | 1952 | mU*mU*mA*mA*mU*A*A | 2304 | XXXXXXXXX | 5-11-4 gapmer | rs7685686 |
| | TCATCACCA | | *A*T*T*G*T*C*A*T*C*m | | XXXXXXXXXX | full PS | (A/G) |
| | | | A*mC*mC*mA | | | | |
| WV-2047 | AUUAATAAATT | 1953 | mA*mU*mU*mA*mA*T*A* | 2305 | XXXXXXXXX | 5-15 hemimer | rs7685686 |
| | GTCATCACC | | A*A*T*T*G*T*C*A*T*C* | | XXXXXXXXXX | full PS | (A/G) |
| | | | A*C*C | | | | |
| WV-2048 | AUUAATAAATT | 1954 | mA*mU*mU*mA*mA*T*A* | 2306 | XXXXXXXXX | 5-14-1 gapmer | rs7685686 |
| | GTCATCACC | | A*A*T*T*G*T*C*A*T*C* | | XXXXXXXXXX | full PS | (A/G) |
| | | | A*C*mC | | | | |
| WV-2049 | AUUAATAAATT | 1955 | mA*mU*mU*mA*mA*T*A* | 2307 | XXXXXXXXX | 5-13-2 gapmer | rs7685686 |
| | GTCATCACC | | A*A*T*T*G*T*C*A*T*C* | | XXXXXXXXXX | full PS | (A/G) |
| | | | A*mC*mC | | | | |
| WV-2050 | AUUAATAAATT | 1956 | mA*mU*mU*mA*mA*T*A* | 2308 | XXXXXXXXX | 5-12-3 gapmer | rs7685686 |
| | GTCATCACC | | A*A*T*T*G*T*C*A*T*C* | | XXXXXXXXXX | full PS | (A/G) |
| | | | mA*mC*mC | | | | |
| WV-2051 | UAUUAATAAAT | 1957 | mU*mA*mU*mU*mA*A*T* | 2309 | XXXXXXXXX | 5-15 hemimer | rs7685686 |
| | TGTCATCAC | | A*A*A*T*T*G*T*C*A*T* | | XXXXXXXXXX | full PS | (A/G) |
| | | | C*A*C | | | | |
| WV-2052 | UAUUAATAAAT | 1958 | mU*mA*mU*mU*mA*A*T* | 2310 | XXXXXXXXX | 5-14-1 gapmer | rs7685686 |
| | TGTCATCAC | | A*A*A*T*T*G*T*C*A*T* | | XXXXXXXXXX | full PS | (A/G) |
| | | | C*A*mC | | | | |
| WV-2053 | UAUUAATAAAT | 1959 | mU*mA*mU*mU*mA*A*T* | 2311 | XXXXXXXXX | 5-13-2 gapmer | rs7685686 |
| | TGTCATCAC | | A*A*A*T*T*G*T*C*A*T* | | XXXXXXXXXX | full PS | (A/G) |
| | | | C*mA*mC | | | | |
| WV-2054 | CUAUUAATAAA | 1960 | mC*mU*mA*mU*mU*A*A | 2312 | XXXXXXXXX | 5-15 hemimer | rs7685686 |
| | TTGTCATCA | | *T*A*A*A*T*T*G*T*C*A* | | XXXXXXXXXX | full PS | (A/G) |
| | | | T*C*A | | | | |
| WV-2055 | CUAUUAATAAA | 1961 | mC*mU*mA*mU*mU*A*A | 2313 | XXXXXXXXX | 5-14-1 gapmer | rs7685686 |
| | TTGTCATCA | | *T*A*A*A*T*T*G*T*C*A* | | XXXXXXXXXX | full PS | (A/G) |
| | | | T*C*mA | | | | |
| WV-2056 | ACUAUTAATAA | 1962 | mA*mC*mU*mA*mU*T*A* | 2314 | XXXXXXXXX | 5-15 hemimer | rs7685686 |
| | ATTGTCATC | | A*T*A*A*A*T*T*G*T*C* | | XXXXXXXXXX | full PS | (A/G) |
| | | | A*T*C | | | | |
| WV-2057 | TGTCATCACCA | 1963 | T*G*T*C*A*T*C*A*C*C* | 2315 | XXXXXXXXX | 15-5 hemimer 1 | rs7685686 |
| | GAAAAAGUC | | A*G*A*A*A*mAmAmGmU | | XXXXXXOOOX | PS on each end | (A/G) |
| | | | *mC | | | and between dN- | |
| | | | | | | mN and dN-dN | |
| WV-2058 | UTGTCATCACC | 1964 | mU*T*G*T*C*A*T*C*A*C | 2316 | XXXXXXXXX | 1-14-5 gapmer 1 | rs7685686 |
| | AGAAAAAGU | | *C*A*G*A*A*mAmAmAm | | XXXXXXOOOX | PS on each end | (A/G) |
| | | | G*mU | | | and between dN- | |
| | | | | | | mN and dN-dN | |
| WV-2059 | TTGTCATCACC | 1965 | T*T*G*T*C*A*T*C*A*C*C | 2317 | XXXXXXXXX | 15-5 hemimer 1 | rs7685686 |
| | AGAAAAAGU | | *A*G*A*A*mAmAmAmG* | | XXXXXXOOOX | PS on each end | (A/G) |
| | | | mU | | | and between dN- | |
| | | | | | | mN and dN-dN | |
| WV-2060 | AUTGTCATCAC | 1966 | mA*mU*T*G*T*C*A*T*C* | 2318 | XXXXXXXXX | 2-13-5 gapmer 1 | rs7685686 |
| | CAGAAAAAG | | A*C*C*A*G*A*mAmAmA | | XXXXXXOOOX | PS on each end | (A/G) |
| | | | mA*mG | | | and between dN- | |
| | | | | | | mN and dN-dN | |
| WV-2061 | ATTGTCATCAC | 1967 | mA*T*T*G*T*C*A*T*C*A | 2319 | XXXXXXXXX | 1-14-5 gapmer 1 | rs7685686 |
| | CAGAAAAAG | | *C*C*A*G*A*mAmAmAm | | XXXXXXOOOX | PS on each end | (A/G) |
| | | | A*mG | | | and between dN- | |
| | | | | | | mN and dN-dN | |
| WV-2062 | AAUTGTCATCA | 1968 | mA*mAmU*T*G*T*C*A*T | 2320 | XOXXXXXXX | 3-12-5 gapmer 1 | rs7685686 |
| | CCAGAAAAA | | *C*A*C*C*A*G*mAmAmA | | XXXXXXOOOX | PS on each end | (A/G) |
| | | | mA*mA | | | and between dN- | |
| | | | | | | mN and dN-dN | |
| WV-2063 | AATTGTCATCA | 1969 | mA*mA*T*T*G*T*C*A*T* | 2321 | XXXXXXXXX | 2-13-5 gapmer 1 | rs7685686 |
| | CCAGAAAAA | | C*A*C*C*A*G*mAmAmA | | XXXXXXOOOX | PS on each end | (A/G) |
| | | | mA*mA | | | and between dN- | |
| | | | | | | mN and dN-dN | |
| WV-2064 | AAATTGTCATC | 1970 | mA*mAmA*T*T*G*T*C*A | 2322 | XOXXXXXXX | 3-12-5 gapmer 1 | rs7685686 |
| | ACCAGAAAA | | *T*C*A*C*C*A*mGmAmA | | XXXXXXOOOX | PS on each end | (A/G) |
| | | | mA*mA | | | and between dN- | |
| | | | | | | mN and dN-dN | |
| WV-2065 | AAAUTGTCATC | 1971 | mA*mAmAmU*T*G*T*C*A | 2323 | XOOXXXXXX | 4-11-5 gapmer 1 | rs7685686 |
| | ACCAGAAAA | | *T*C*A*C*C*A*mGmAmA | | XXXXXXOOOX | PS on each end | (A/G) |
| | | | mA*mA | | | and between dN- | |
| | | | | | | mN and dN-dN | |
| WV-2066 | UAAAUTGTCAT | 1972 | mU*mAmAmAmU*T*G*T* | 2324 | XOOOXXXXX | 5-11-4 gapmer 1 | rs7685686 |
| | CACCAGAAA | | C*A*T*C*A*C*C*A*mGm | | XXXXXXXOOX | PS on each end | (A/G) |
| | | | AmA*mA | | | and between dN- | |
| | | | | | | mN and dN-dN | |
| WV-2067 | UAAAUTGTCAT | 1973 | mU*mAmAmAmU*T*G*T* | 2325 | XOOOXXXXX | 5-10-5 gapmer 1 | rs7685686 |
| | CACCAGAAA | | C*A*T*C*A*C*C*mAmGm | | XXXXXXOOOX | PS on each end | (A/G) |
| | | | AmA*mA | | | and between dN- | |
| | | | | | | mN and dN-dN | |
| WV-2068 | AUAAATTGTCA | 1974 | mA*mUmAmAmA*T*T*G* | 2326 | XOOOXXXXX | 5-11-4 gapmer 1 | rs7685686 |
| | TCACCAGAA | | T*C*A*T*C*A*C*C*mAm | | XXXXXXXOOX | PS on each end | (A/G) |
| | | | GmA*mA | | | and between dN- | |
| | | | | | | mN and dN-dN | |
| WV-2069 | AUAAATTGTCA | 1975 | mA*mUmAmAmA*T*T*G* | 2327 | XOOOXXXXX | 5-10-5 gapmer 1 | rs7685686 |
| | TCACCAGAA | | T*C*A*T*C*A*C*mCmAm | | XXXXXXOOOX | PS on each end | (A/G) |
| | | | GmA*mA | | | and between dN- | |
| | | | | | | mN and dN-dN | |
| WV-2070 | AAUAAATTGTC | 1976 | mA*mAmUmAmA*A*T*T* | 2328 | XOOOXXXXX | 5-12-3 gapmer 1 | rs7685686 |
| | ATCACCAGA | | G*T*C*A*T*C*A*C*C*mA | | XXXXXXXXOX | PS on each end | (A/G) |
| | | | mG*mA | | | and between dN- | |
| | | | | | | mN and dN-dN | |
| WV-2071 | AAUAAATTGTC | 1977 | mA*mAmUmAmA*A*T*T* | 2329 | XOOOXXXXX | 5-11-4 gapmer 1 | rs7685686 |
| | ATCACCAGA | | G*T*C*A*T*C*A*C*mCm | | XXXXXXXOOX | PS on each end | (A/G) |
| | | | AmG*mA | | | and between dN- | |
| | | | | | | mN and dN-dN | |
| WV-2072 | AAUAAATTGTC | 1978 | mA*mAmUmAmA*A*T*T* | 2330 | XOOOXXXXX | 5-10-5 gapmer 1 | rs7685686 |
| | ATCACCAGA | | G*T*C*A*T*C*A*mCmCm | | XXXXXXOOOX | PS on each end | (A/G) |
| | | | AmG*mA | | | and between dN- | |
| | | | | | | mN and dN-dN | |
| WV-2073 | UAAUAAATTGT | 1979 | mU*mAmAmUmA*A*A*T* | 2331 | XOOOXXXXX | 5-13-2 gapmer 1 | rs7685686 |
| | CATCACCAG | | T*G*T*C*A*T*C*A*C*C* | | XXXXXXXXXX | PS on each end | (A/G) |
| | | | mA*mG | | | and between dN- | |
| | | | | | | mN and dN-dN | |
| WV-2074 | UAAUAAATTGT | 1980 | mU*mAmAmUmA*A*A*T* | 2332 | XOOOXXXXX | 5-12-3 gapmer 1 | rs7685686 |
| | CATCACCAG | | T*G*T*C*A*T*C*A*C*mC | | XXXXXXXXOX | PS on each end | (A/G) |
| | | | mA*mG | | | and between dN- | |
| | | | | | | mN and dN-dN | |
| WV-2075 | UAAUAAATTGT | 1981 | mU*mAmAmUmA*A*A*T* | 2333 | XOOOXXXXX | 5-11-4 gapmer 1 | rs7685686 |
| | CATCACCAG | | T*G*T*C*A*T*C*A*mCmC | | XXXXXXXOOX | PS on each end | (A/G) |
| | | | mA*mG | | | and between dN- | |
| | | | | | | mN and dN-dN | |
| WV-2076 | UAAUAAATTGT | 1982 | mU*mAmAmUmA*A*A*T* | 2334 | XOOOXXXXX | 5-10-5 gapmer 1 | rs7685686 |
| | CATCACCAG | | T*G*T*C*A*T*C*mAmCm | | XXXXXXOOOX | PS on each end | (A/G) |
| | | | CmA*mG | | | and between dN- | |
| | | | | | | mN and dN-dN | |
| WV-2077 | UUAAUAAATTG | 1983 | mU*mUmAmAmU*A*A*A* | 2335 | XOOOXXXXX | 5-14-1 gapmer 1 | rs7685686 |
| | TCATCACCA | | T*T*G*T*C*A*T*C*A*C*C | | XXXXXXXXXX | PS on each end | (A/G) |
| | | | *mA | | | and between dN- | |
| | | | | | | mN and dN-dN | |
| WV-2078 | UUAAUAAATTG | 1984 | mU*mUmAmAmU*A*A*A* | 2336 | XOOOXXXXX | 5-13-2 gapmer 1 | rs7685686 |
| | TCATCACCA | | T*T*G*T*C*A*T*C*A*C* | | XXXXXXXXXX | PS on each end | (A/G) |
| | | | mC*mA | | | and between dN- | |
| | | | | | | mN and dN-dN | |
| WV-2079 | UUAAUAAATTG | 1985 | mU*mUmAmAmU*A*A*A* | 2337 | XOOOXXXXX | 5-12-3 gapmer 1 | rs7685686 |
| | TCATCACCA | | T*T*G*T*C*A*T*C*A*mC | | XXXXXXXXOX | PS on each end | (A/G) |
| | | | mC*mA | | | and between dN- | |
| | | | | | | mN and dN-dN | |
| WV-2080 | UUAAUAAATTG | 1986 | mU*mUmAmAmU*A*A*A* | 2338 | XOOOXXXXX | 5-11-4 gapmer 1 | rs7685686 |
| | TCATCACCA | | T*T*G*T*C*A*T*C*mAmC | | XXXXXXXOOX | PS on each end | (A/G) |
| | | | mC*mA | | | and between dN- | |
| | | | | | | mN and dN-dN | |
| WV-2081 | AUUAATAAATT | 1987 | mA*mUmUmAmA*T*A*A* | 2339 | XOOOXXXXX | 5-15 hemimer 1 | rs7685686 |
| | GTCATCACC | | A*T*T*G*T*C*A*T*C*A*C | | XXXXXXXXXX | PS on each end | (A/G) |
| | | | *C | | | and between dN- | |
| | | | | | | mN and dN-dN | |
| WV-2082 | AUUAATAAATT | 1988 | mA*mUmUmAmA*T*A*A* | 2340 | XOOOXXXXX | 5-14-1 gapmer 1 | rs7685686 |
| | GTCATCACC | | A*T*T*G*T*C*A*T*C*A*C | | XXXXXXXXXX | PS on each end | (A/G) |
| | | | *mC | | | and between dN- | |
| | | | | | | mN and dN-dN | |
| WV-2083 | AUUAATAAATT | 1989 | mA*mUmUmAmA*T*A*A* | 2341 | XOOOXXXXX | 5-13-2 gapmer 1 | rs7685686 |
| | GTCATCACC | | A*T*T*G*T*C*A*T*C*A* | | XXXXXXXXXX | PS on each end | (A/G) |
| | | | mC*mC | | | and between dN- | |
| | | | | | | mN and dN-dN | |
| WV-2084 | AUUAATAAATT | 1990 | mA*mUmUmAmA*T*A*A* | 2342 | XOOOXXXXX | 5-12-3 gapmer 1 | rs7685686 |
| | GTCATCACC | | A*T*T*G*T*C*A*T*C*mA | | XXXXXXXXOX | PS on each end | (A/G) |
| | | | mC*mC | | | and between dN- | |
| | | | | | | mN and dN-dN | |
| WV-2085 | UAUUAATAAAT | 1991 | mU*mAmUmUmA*A*T*A* | 2343 | XOOOXXXXX | 5-15 hemimer 1 | rs7685686 |
| | TGTCATCAC | | A*A*T*T*G*T*C*A*T*C* | | XXXXXXXXXX | PS on each end | (A/G) |
| | | | A*C | | | and between dN- | |
| | | | | | | mN and dN-dN | |
| WV-2086 | UAUUAATAAAT | 1992 | mU*mAmUmUmA*A*T*A* | 2344 | XOOOXXXXX | 5-14-1 gapmer 1 | rs7685686 |
| | TGTCATCAC | | A*A*T*T*G*T*C*A*T*C* | | XXXXXXXXXX | PS on each end | (A/G) |
| | | | A*mC | | | and between dN- | |
| | | | | | | mN and dN-dN | |
| WV-2087 | UAUUAATAAAT | 1993 | mU*mAmUmUmA*A*T*A* | 2345 | XOOOXXXXX | 5-13-2 gapmer 1 | rs7685686 |
| | TGTCATCAC | | A*A*T*T*G*T*C*A*T*C* | | XXXXXXXXXX | PS on each end | (A/G) |
| | | | mA*mC | | | and between dN- | |
| | | | | | | mN and dN-dN | |
| WV-2088 | CUAUUAATAAA | 1994 | mC*mUmAmUmU*A*A*T* | 2346 | XOOOXXXXX | 5-15 hemimer 1 | rs7685686 |
| | TTGTCATCA | | A*A*A*T*T*G*T*C*A*T* | | XXXXXXXXXX | PS on each end | (A/G) |
| | | | C*A | | | and between dN- | |
| | | | | | | mN and dN-dN | |
| WV-2089 | CUAUUAATAAA | 1995 | mC*mUmAmUmU*A*A*T* | 2347 | XOOOXXXXX | 5-14-1 gapmer 1 | rs7685686 |
| | TTGTCATCA | | A*A*A*T*T*G*T*C*A*T* | | XXXXXXXXXX | PS on each end | (A/G) |
| | | | C*mA | | | and between dN- | |
| | | | | | | mN and dN-dN | |
| WV-2090 | ACUAUTAATAA | 1996 | mA*mCmUmAmU*T*A*A* | 2348 | XOOOXXXXX | 5-15 hemimer 1 | rs7685686 |
| | ATTGTCATC | | T*A*A*A*T*T*G*T*C*A*T | | XXXXXXXXXX | PS on each end | (A/G) |
| | | | *C | | | and between dN- | |
| | | | | | | mN and dN-dN | |
| WV-2163 | GACUUUUUCU | 1997 | rGrArCrUrUrUrUrUrCrUrGr | 2349 | OOOOOOOOO | HTT rs7685686 | HTT rs7685686 |
| | GGUGAUGGCA | | GrUrGrArUrGrGrCrArArUrU | | OOOOOOOOO | | |
| | AUUUAUUAAU | | rUrArUrUrArArUrArG | | OOOOOOOOO | | |
| | AG | | | | OOOO | | |
| WV-2164 | GACUUUUUCU | 1998 | rGrArCrUrUrUrUrUrCrUrGr | 2350 | OOOOOOOOO | HTT rs7685686 | HTT rs7685686 |
| | GGUGAUGACA | | GrUrGrArUrGrArCrArArUrU | | OOOOOOOOO | | |
| | AUUUAUUAAU | | rUrArUrUrArArUrArG | | OOOOOOOOO | | |
| | AG | | | | OOOO | | |
| WV-2269 | UAAAUTGTCAT | 1999 | mU*SmAmAmAmU*ST*SG | 2351 | SOOOSSSSSRS | 5-10-5 2′ OMe- | HTT rs7685686 |
| | CACCAGAAA | | *ST*SC*SA*RT*SC*SA*SC | | SSSSOOOS | DNA-2′-OMe | |
| | | | *SC*SmAmGmAmA*SmA | | | Gapmer 1-3-11- | |
| | | | | | | 3-1 (PS/PO) | |
| WV-2270 | AUAAATTGTCA | 2000 | mA*SmUmAmAmA*ST*ST | 2352 | SOOOSSSSSSR | 5-10-5 2′ OMe- | HTT rs7685686 |
| | TCACCAGAA | | *SG*ST*SC*SA*RT*SC*SA | | SSSSOOOS | DNA-2′-OMe | |
| | | | *SC*SmCmAmGmA*SmA | | | Gapmer 1-3-11- | |
| | | | | | | 3-1 (PS/PO) | |
| WV-2271 | AAUAAATTGTC | 2001 | mA*SmAmUmAmA*SA*ST | 2353 | SOOOSSSSSSS | 5-10-5 2′ OMe- | HTT rs7685686 |
| | ATCACCAGA | | *ST*SG*ST*SC*SA*RT*SC | | RSSSOOOS | DNA-2′-OMe | |
| | | | *SA*SmCmCmAmG*SmA | | | Gapmer 1-3-11- | |
| | | | | | | 3-1 (PS/PO) | |
| WV-2272 | UAAUAAATTGT | 2002 | mU*SmAmAmUmA*SA*SA | 2354 | SOOOSSSSSSS | 5-10-5 2′ OMe- | HTT rs7685686 |
| | CATCACCAG | | *ST*ST*SG*ST*SC*SA*RT | | SRSSOOOS | DNA-2′-OMe | |
| | | | *SC*SmAmCmCmA*SmG | | | Gapmer 1-3-11- | |
| | | | | | | 3-1 (PS/PO) | |
| WV-2374 | AAUAAATTGTC | 2003 | mA*SmAmUmAmA*SA*ST | 2355 | SOOOSSSSSSS | P10 stereopure | HTT rs7685686 |
| | ATCACCAGA | | *ST*SG*ST*SC*SA*RT*SC | | RSSSSOOS | analogue of WV- | |
| | | | *SA*SC*SmCmAmG*SmA | | | 2071 5-11-4 2′- | |
| | | | | | | OMe-DNA-2′- | |
| | | | | | | OMe Gapmer 1- | |
| | | | | | | 3-12-2-1 | |
| | | | | | | (PS/PO) | |
| WV-2375 | UAAUAAATTGT | 2004 | mU*SmAmAmUmA*SA*SA | 2356 | SOOOSSSSSSS | P11 stereopure | HTT rs7685686 |
| | CATCACCAG | | *ST*ST*SG*ST*SC*SA*RT | | SRSSSOOS | analogue of WV- | |
| | | | *SC*SA*SmCmCmA*SmG | | | 20755-11-4 2′- | |
| | | | | | | OMe-DNA-2′- | |
| | | | | | | OMe Gapmer 1- | |
| | | | | | | 3-12-2-1 | |
| | | | | | | (PS/PO) | |
| WV-2377 | GCACAAGGGCA | 2005 | mG*mCmAmCmA*A*G*G* | 2357 | XOOOXXXXX | P11 | HTT rs362307 |
| | CAGACUUCC | | G*C*A*C*A*G*A*mCmUm | | XXXXXXOOOX | stereorandom | |
| | | | UmC*mC | | | analogue of WV- | |
| | | | | | | 932 5-10-5 2′- | |
| | | | | | | OMe-DNA-2′- | |
| | | | | | | OMe Gapmer | |
| | | | | | | and 1-3-11-3-1 | |
| | | | | | | (PS/PO) | |
| WV-2378 | GCACAAGGGCA | 2006 | mG*SmCmAmCmA*SA*SG | 2358 | SOOOSSSSSSS | P11 | HTT rs362307 |
| | CAGACUUCC | | *SG*SG*SC*SA*SC*RA*S | | RSSSOOOS | stereorandom | |
| | | | G*SA*SmCmUmUmC*SmC | | | analogue of WV- | |
| | | | | | | 932 5-10-5 2′- | |
| | | | | | | OMe-DNA-2′- | |
| | | | | | | OMe Gapmer | |
| | | | | | | and 1-3-11-3-1 | |
| | | | | | | (PS/PO) | |
| WV-2379 | CACAAGGGCAC | 2007 | mC*mAmCmAmA*G*G*G* | 2359 | XOOOXXXXX | P10 | HTT rs362307 |
| | AGACUUCCA | | C*A*C*A*G*A*C*mUmUm | | XXXXXXOOOX | sereorandom | |
| | | | CmC*mA | | | analogue of WV- | |
| | | | | | | 933 5-10-5 2′- | |
| | | | | | | OMe-DNA-2′- | |
| | | | | | | OMe Gapmer | |
| | | | | | | and 1-3-11-3-1 | |
| | | | | | | (PS/PO) | |
| WV-2380 | CACAAGGGCAC | 2008 | mC*SmAmCmAmA*SG*SG | 2360 | SOOOSSSSSSR | P10 stereopure | HTT rs362307 |
| | AGACUUCCA | | *SG*SC*SA*SC*RA*SG*S | | SSSSOOOS | analogue of WV- | |
| | | | A*SC*SmUmUmCmC*SmA | | | 933 5-10-5 2′- | |
| | | | | | | OMe-DNA-2′- | |
| | | | | | | OMe Gapmer | |
| | | | | | | and 1-3-11-3-1 | |
| | | | | | | (PS/PO) | |
| WV-2416 | UAAAUTGTCAT | 2009 | mU*SmAmAmAmU*ST*SG | 2361 | SOOOSSSSRSS | P8 5-10-5 2′ | HTT rs7685686 |
| | CACCAGAAA | | *ST*SC*RA*ST*SC*SA*SC | | SSSSOOOS | OMe-DNA-2′- | |
| | | | *SC*SmAmGmAmA*SmA | | | OMe Gapmer 1- | |
| | | | | | | 3-11-3-1 | |
| | | | | | | (PS/PO) | |
| WV-2417 | AUAAATTGTCA | 2010 | mA*SmUmAmAmA*ST*ST | 2362 | SOOOSSSSSRS | P9 5-10-5 2′ | HTT rs7685686 |
| | TCACCAGAA | | *SG*ST*SC*RA*ST*SC*SA | | SSSSOOOS | OMe-DNA-2′- | |
| | | | *SC*SmCmAmGmA*SmA | | | OMe Gapmer 1- | |
| | | | | | | 3-11-3-1 | |
| | | | | | | (PS/PO) | |
| WV-2418 | AAUAAATTGTC | 2011 | mA*SmAmUmAmA*SA*ST | 2363 | SOOOSSSSSSR | P10 5-10-5 2′ | HTT rs7685686 |
| | ATCACCAGA | | *ST*SG*ST*SC*RA*ST*SC | | SSSSOOOS | OMe-DNA-2′- | |
| | | | *SA*SmCmCmAmG*SmA | | | OMe Gapmer 1- | |
| | | | | | | 3-11-3-1 | |
| | | | | | | (PS/PO) | |
| WV-2419 | UAAUAAATTGT | 2012 | mU*SmAmAmUmA*SA*SA | 2364 | SOOOSSSSSSS | P11 5-10-5 2′ | HTT rs7685686 |
| | CATCACCAG | | *ST*ST*SG*ST*SC*RA*ST | | RSSSOOOS | OMe-DNA-2′- | |
| | | | *SC*SmAmCmCmA*SmG | | | OMe Gapmer 1- | |
| | | | | | | 3-11-3-1 | |
| | | | | | | (PS/PO) | |
| WV-2589 | UCCCCACAGAG | 2013 | mU*SmCmCmCmC*SA*SC | 2365 | SOOOSSRSSSS | P6 5-10-5 (2′- | HTT rs2530595 |
| | GGAGGAAGC | | *RA*SG*SA*SG*SG*SG*S | | SSSSOOOS | OMe-DNA-2′- | (C/T) |
| | | | A*SG*SmGmAmAmG*SmC | | | OMe) 1-3-11-3- | |
| | | | | | | 1 (PS/PO) | |
| | | | | | | Gapmer | |
| WV-2590 | CUCCCCACAGA | 2014 | mC*SmUmCmCmC*SC*SA | 2366 | SOOOSSSRSSS | P7 5-10-5 (2′- | HTT |
| | GGGAGGAAG | | *SC*RA*SG*SA*SG*SG*S | | SSSSOOOS | OMe-DNA-2′- | rs2530595 |
| | | | G*SA*SmGmGmAmA*SmG | | | OMe) 1-3-11-3- | (C/T) |
| | | | | | | 1 (PS/PO) | |
| | | | | | | Gapmer | |
| WV-2591 | CCUCCCCACAG | 2015 | mC*SmCmUmCmC*SC*SC* | 2367 | SOOOSSSSRSS | P8 5-10-5 (2′- | HTT |
| | AGGGAGGAA | | SA*SC*RA*SG*SA*SG*SG | | SSSSOOOS | OMe-DNA-2′- | rs2530595 |
| | | | *SG*SmAmGmGmA*SmA | | | OMe) 1-3-11-3- | (C/T) |
| | | | | | | 1 (PS/PO) | |
| | | | | | | Gapmer | |
| WV-2592 | UCCUCCCCACA | 2016 | mU*SmCmCmUmC*SC*SC | 2368 | SOOOSSSSSRS | P9 5-10-5 (2′- | HTT |
| | GAGGGAGGA | | *SC*SA*SC*RA*SG*SA*S | | SSSSOOOS | OMe-DNA-2′- | rs2530595 |
| | | | G*SG*SmGmAmGmG*SmA | | | OMe) 1-3-11-3- | (C/T) |
| | | | | | | 1 (PS/PO) | |
| | | | | | | Gapmer | |
| WV-2593 | GUCCUCCCCAC | 2017 | mG*SmUmCmCmU*SC*SC | 2369 | SOOOSSSSSSR | P10 5-10-5 (2′- | HTT |
| | AGAGGGAGG | | *SC*SC*SA*SC*RA*SG*S | | SSSSOOOS | OMe-DNA-2′- | rs2530595 |
| | | | A*SG*SmGmGmAmG*SmG | | | OMe) 1-3-11-3- | (C/T) |
| | | | | | | 1 (PS/PO) | |
| | | | | | | Gapmer | |
| WV-2594 | GGUCCTCCCCA | 2018 | mG*SmGmUmCmC*ST*SC | 2370 | SOOOSSSSSSS | P11 5-10-5 (2′- | HTT |
| | CAGAGGGAG | | *SC*SC*SC*SA*SC*RA*S | | RSSSOOOS | OMe-DNA-2′- | rs2530595 |
| | | | G*SA*SmGmGmGmA*SmG | | | OMe) 1-3-11-3- | (C/T) |
| | | | | | | 1 (PS/PO) | |
| | | | | | | Gapmer | |
| WV-2595 | GGGUCCTCCCC | 2019 | mG*SmGmGmUmC*SC*ST | 2371 | SOOOSSSSSSS | P12 5-10-5 (2′- | HTT |
| | ACAGAGGGA | | *SC*SC*SC*SC*SA*SC*RA | | SRSSOOOS | OMe-DNA-2′- | rs2530595 |
| | | | *SG*SmAmGmGmG*SmA | | | OMe) 1-3-11-3- | (C/T) |
| | | | | | | 1 (PS/PO) | |
| | | | | | | Gapmer | |
| WV-2596 | CGGGUCCTCCC | 2020 | mC*SmGmGmGmU*SC*SC | 2372 | SOOOSSSSSSS | P13 5-10-5 (2′- | HTT |
| | CACAGAGGG | | *ST*SC*SC*SC*SC*SA*SC | | SSRSOOOS | OMe-DNA-2′- | rs2530595 |
| | | | *RA*SmGmAmGmG*SmG | | | OMe) 1-3-11-3- | (C/T) |
| | | | | | | 1 (PS/PO) | |
| | | | | | | Gapmer | |
| WV-2597 | ACAGUAGATGA | 2021 | mA*SmCmAmGmU*SA*SG | 2373 | SOOOSSRSSSS | P6 5-10-5 (2′- | HTT |
| | GGGAGCAGG | | *RA*ST*SG*SA*SG*SG*S | | SSSSOOOS | OMe-DNA-2′- | (rs362331) |
| | | | G*SA*SmGmCmAmG*SmG | | | OMe) 1-3-11-3- | (C/T) |
| | | | | | | 1 (PS/PO) | |
| | | | | | | Gapmer | |
| WV-2598 | CACAGTAGATG | 2022 | mC*SmAmCmAmG*ST*SA | 2374 | SOOOSSSRSSS | P7 5-10-5 (2′- | HTT |
| | AGGGAGCAG | | *SG*RA*ST*SG*SA*SG*S | | SSSSOOOS | OMe-DNA-2′- | (rs362331) |
| | | | G*SG*SmAmGmCmA*SmG | | | OMe) 1-3-11-3- | (C/T) |
| | | | | | | 1 (PS/PO) | |
| | | | | | | Gapmer | |
| WV-2599 | ACACAGTAGAT | 2023 | mA*SmCmAmCmA*SG*ST | 2375 | SOOOSSSSRSS | P8 5-10-5 (2′- | HTT |
| | GAGGGAGCA | | *SA*SG*RA*ST*SG*SA*S | | SSSSOOOS | OMe-DNA-2′- | (rs362331) |
| | | | G*SG*SmGmAmGmC*SmA | | | OMe) 1-3-11-3- | (C/T) |
| | | | | | | 1 (PS/PO) | |
| | | | | | | Gapmer | |
| WV-2600 | CACACAGTAGA | 2024 | mC*SmAmCmAmC*SA*SG | 2376 | SOOOSSSSSRS | P9 5-10-5 (2′- | HTT |
| | TGAGGGAGC | | *ST*SA*SG*RA*ST*SG*S | | SSSSOOOS | OMe-DNA-2′- | (rs362331) |
| | | | A*SG*SmGmGmAmG*SmC | | | OMe) 1-3-11-3- | (C/T) |
| | | | | | | 1 (PS/PO) | |
| | | | | | | Gapmer | |
| WV-2601 | GCACACAGTAG | 2025 | mG*SmCmAmCmA*SC*SA | 2377 | SOOOSSSSSSR | P10 5-10-5 (2′- | HTT |
| | ATGAGGGAG | | *SG*ST*SA*SG*RA*ST*S | | SSSSOOOS | OMe-DNA-2′- | (rs362331) |
| | | | G*SA*SmGmGmGmA*SmG | | | OMe) 1-3-11-3- | (C/T) |
| | | | | | | 1 (PS/PO) | |
| | | | | | | Gapmer | |
| WV-2602 | UGCACACAGTA | 2026 | mU*SmGmCmAmC*SA*SC | 2378 | SOOOSSSSSSS | P11 5-10-5 (2′- | HTT |
| | GATGAGGGA | | *SA*SG*ST*SA*SG*RA*S | | RSSSOOOS | OMe-DNA-2′- | (rs362331) |
| | | | T*SG*SmAmGmGmG*SmA | | | OMe) 1-3-11-3- | (C/T) |
| | | | | | | 1 (PS/PO) | |
| | | | | | | Gapmer | |
| WV-2603 | GUGCACACAGT | 2027 | mG*SmUmGmCmA*SC*SA | 2379 | SOOOSSSSSSS | P12 5-10-5 (2′- | HTT |
| | AGATGAGGG | | *SC*SA*SG*ST*SA*SG*R | | SRSSOOOS | OMe-DNA-2′- | (rs362331) |
| | | | A*ST*SmGmAmGmG*SmG | | | OMe) 1-3-11-3- | (C/T) |
| | | | | | | 1 (PS/PO) | |
| | | | | | | Gapmer | |
| WV-2604 | AGUGCACACAG | 2028 | mA*SmGmUmGmC*SA*SC | 2380 | SOOOSSSSSSS | P13 5-10-5 (2′- | HTT |
| | TAGAUGAGG | | *SA*SC*SA*SG*ST*SA*SG | | SSRSOOOS | OMe-DNA-2′- | (rs362331) |
| | | | *RA*SmUmGmAmG*SmG | | | OMe) 1-3-11-3- | (C/T) |
| | | | | | | 1 (PS/PO) | |
| | | | | | | Gapmer | |
| WV-2605 | UCCCCACAGAG | 2029 | mU*mCmCmCmC*A*C*A* | 2381 | XOOOXXXXX | P6 5-10-5 (2′- | HTT r2530595 |
| | GGAGGAAGC | | G*A*G*G*G*A*G*mGmAm | | XXXXXXOOOX | OMe-DNA-2′- | (C/T) |
| | | | AmG*mC | | | OMe) 1-3-11-3- | |
| | | | | | | 1 (P/PO) Gapmer | |
| WV-2606 | CUCCCCACAGA | 2030 | mC*mUmCmCmC*C*A*C* | 2382 | XOOOXXXXX | P7 5-10-5 (2′- | HTT r2530595 |
| | GGGAGGAAG | | A*G*A*G*G*G*A*mGmGm | | XXXXXXOOOX | OMe-DNA-2′- | (C/T) |
| | | | AmA*mG | | | OMe) 1-3-11-3- | |
| | | | | | | 1 (P/PO) Gapmer | |
| WV-2607 | CCUCCCCACAG | 2031 | mC*mCmUmCmC*C*C*A* | 2383 | XOOOXXXXX | P8 5-10-5 (2′- | HTT r2530595 |
| | AGGGAGGAA | | C*A*G*A*G*G*G*mAmGm | | XXXXXXOOOX | OMe-DNA-2′- | (C/T) |
| | | | GmA*mA | | | OMe) 1-3-11-3- | |
| | | | | | | 1 (P/PO) Gapmer | |
| WV-2608 | UCCUCCCCACA | 2032 | mU*mCmCmUmC*C*C*C* | 2384 | XOOOXXXXX | P9 5-10-5 (2′- | HTT r2530595 |
| | GAGGGAGGA | | A*C*A*G*A*G*G*mGmAm | | XXXXXXOOOX | OMe-DNA-2′- | (C/T) |
| | | | GmG*mA | | | OMe) 1-3-11-3- | |
| | | | | | | 1 (P/PO) Gapmer | |
| WV-2609 | GUCCUCCCCAC | 2033 | mG*mUmCmCmU*C*C*C* | 2385 | XOOOXXXXX | P10 5-10-5 (2′- | HTT r2530595 |
| | AGAGGGAGG | | C*A*C*A*G*A*G*mGmGm | | XXXXXXOOOX | OMe-DNA-2′- | (C/T) |
| | | | AmG*mG | | | OMe) 1-3-11-3- | |
| | | | | | | 1 (P/PO) Gapmer | |
| WV-2610 | GGUCCTCCCCA | 2034 | mG*mGmUmCmC*T*C*C* | 2386 | XOOOXXXXX | P11 5-10-5 (2′- | HTT r2530595 |
| | CAGAGGGAG | | C*C*A*C*A*G*A*mGmGm | | XXXXXXOOOX | OMe-DNA-2′- | (C/T) |
| | | | GmA*mG | | | OMe) 1-3-11-3- | |
| | | | | | | 1 (P/PO) Gapmer | |
| WV-2611 | GGGUCCTCCCC | 2035 | mG*mGmGmUmC*C*T*C* | 2387 | XOOOXXXXX | P12 5-10-5 (2′- | HTT r2530595 |
| | ACAGAGGGA | | C*C*C*A*C*A*G*mAmGm | | XXXXXXOOOX | OMe-DNA-2′- | (C/T) |
| | | | GmG*mA | | | OMe) 1-3-11-3- | |
| | | | | | | 1 (P/PO) Gapmer | |
| WV-2612 | CGGGUCCTCCC | 2036 | mC*mGmGmGmU*C*C*T* | 2388 | XOOOXXXXX | P13 5-10-5 (2′- | HTT r2530595 |
| | CACAGAGGG | | C*C*C*C*A*C*A*mGmAm | | XXXXXXOOOX | OMe-DNA-2′- | (C/T) |
| | | | GmG*mG | | | OMe) 1-3-11-3- | |
| | | | | | | 1 (P/PO) Gapmer | |
| WV-2613 | ACAGUAGATGA | 2037 | mA*mCmAmGmU*A*G*A* | 2389 | XOOOXXXXX | P6 5-10-5 (2′- | HTT (r362331) |
| | GGGAGCAGG | | T*G*A*G*G*G*A*mGmCm | | XXXXXXOOOX | OMe-DNA-2′- | (C/T) |
| | | | AmG*mG | | | OMe) 1-3-11-3- | |
| | | | | | | 1 (P/PO) Gapmer | |
| WV-2614 | CACAGTAGATG | 2038 | mC*mAmCmAmG*T*A*G* | 2390 | XOOOXXXXX | P7 5-10-5 (2′- | HTT (r362331) |
| | AGGGAGCAG | | A*T*G*A*G*G*G*mAmGm | | XXXXXXOOOX | OMe-DNA-2′- | (C/T) |
| | | | CmA*mG | | | OMe) 1-3-11-3- | |
| | | | | | | 1 (P/PO) Gapmer | |
| WV-2615 | ACACAGTAGAT | 2039 | mA*mCmAmCmA*G*T*A* | 2391 | XOOOXXXXX | P8 5-10-5 (2′- | HTT (r362331) |
| | GAGGGAGCA | | G*A*T*G*A*G*G*mGmAm | | XXXXXXOOOX | OMe-DNA-2′- | (C/T) |
| | | | GmC*mA | | | OMe) 1-3-11-3- | |
| | | | | | | 1 (P/PO) Gapmer | |
| WV-2616 | CACACAGTAGA | 2040 | mC*mAmCmAmC*A*G*T* | 2392 | XOOOXXXXX | P9 5-10-5 (2′- | HTT (r362331) |
| | TGAGGGAGC | | A*G*A*T*G*A*G*mGmGm | | XXXXXXOOOX | OMe-DNA-2′- | (C/T) |
| | | | AmG*mC | | | OMe) 1-3-11-3- | |
| | | | | | | 1 (P/PO) Gapmer | |
| WV-2617 | GCACACAGTAG | 2041 | mG*mCmAmCmA*C*A*G* | 2393 | XOOOXXXXX | P10 5-10-5 (2′- | HTT (r362331) |
| | ATGAGGGAG | | T*A*G*A*T*G*A*mGmGm | | XXXXXXOOOX | OMe-DNA-2′- | (C/T) |
| | | | GmA*mG | | | OMe) 1-3-11-3- | |
| | | | | | | 1 (P/PO) Gapmer | |
| WV-2618 | UGCACACAGTA | 2042 | mU*mGmCmAmC*A*C*A* | 2394 | XOOOXXXXX | P11 5-10-5 (2′- | HTT (r362331) |
| | GATGAGGGA | | G*T*A*G*A*T*G*mAmGm | | XXXXXXOOOX | OMe-DNA-2′- | (C/T) |
| | | | GmG*mA | | | OMe) 1-3-11-3- | |
| | | | | | | 1 (P/PO) Gapmer | |
| WV-2619 | GUGCACACAGT | 2043 | mG*mUmGmCmA*C*A*C* | 2395 | X000XXXXX | P12 5-10-5 (2′- | HTT (r362331) |
| | AGATGAGGG | | A*G*T*A*G*A*T*mGmAm | | XXXXXXOOOX | OMe-DNA-2′- | (C/T) |
| | | | GmG*mG | | | OMe) 1-3-11-3- | |
| | | | | | | 1 (P/PO) Gapmer | |
| WV-2620 | AGUGCACACAG | 2044 | mA*mGmUmGmC*A*C*A* | 2396 | XOOOXXXXX | P13 5-10-5 (2′- | HTT (r362331) |
| | TAGAUGAGG | | C*A*G*T*A*G*A*mUmGm | | XXXXXXOOOX | OMe-DNA-2′- | (C/T) |
| | | | AmG*mG | | | OMe) 1-3-11-3- | |
| | | | | | | 1 (P/PO) Gapmer | |
| WV-2623 | GGCACAAGGGC | 2045 | GGCACAAGGGCACAGAC | 2397 | OOOOOOOOO | DNA version of | HTT rs362307 |
| | ACAGACTTC | | TTC | | OOOOOOOOOO | WV-1092 | (C/T) |
| WV-2659 | GGCACAAGGGC | 2046 | mG*SmGmCmAmC*SA*SA | 2398 | SOOOSSSSSSS | WV-1092 | rs362307 |
| | ACAGACUUC | | *SG*SG*SG*SC*SA*SC*S | | SSSSOOOS | analogue with | Human HTT |
| | | | A*SG*SmAmCmUmU*SmC | | | All Sp | |
| | | | | | | stereochemistry | |
| WV-2671 | GGGUCCTCCCC | 2047 | mG*SmG*SmGmUmC*SC* | 2399 | SSOOSSSSSSSS | P12 5-10-5 (2′- | HTT |
| | ACAGAGGGA | | ST*SC*SC*SC*SC*SA*SC* | | RSSOOSS | OMe-DNA-2′- | rs2530595 |
| | | | RA*SG*SmAmGmG*SmG* | | | OMe) 2-2-11-2- | (C/T) |
| | | | SmA | | | 2 (PS/PO) | |
| | | | | | | Gapmer with Sp | |
| | | | | | | wings | |
| WV-2672 | GGGUCCTCCCC | 2048 | mG*RmG*RmGmUmC*SC* | 2400 | RROOSSSSSSS | P12 5-10-5 (2′- | HTT |
| | ACAGAGGGA | | ST*SC*SC*SC*SC*SA*SC* | | SRSSOORR | OMe-DNA-2′- | rs2530595 |
| | | | RA*SG*SmAmGmG*RmG* | | | OMe) 4-11-4 | (C/T) |
| | | | RmA | | | (PS/PO) Gapmer | |
| | | | | | | with Rp wings | |
| WV-2673 | GGGUCCTCCCC | 2049 | mG*SmG*SmG*SmU*SmC* | 2401 | SSSSSSSSSSSS | P12 5-10-5 (2′- | HTT |
| | ACAGAGGGA | | SC*ST*SC*SC*SC*SC*SA* | | RSSSSSS | OMe-DNA-2′- | rs2530595 |
| | | | SC*RA*SG*SmA*SmG*Sm | | | OMe) 2-2-11-2- | (C/T) |
| | | | G*SmG*SmA | | | 2 (PS/PO) | |
| | | | | | | Gapmer with Sp | |
| | | | | | | wings | |
| WV-2674 | GGGUCCTCCCC | 2050 | mG*RmG*RmG*RmU*RmC | 2402 | RRRRSSSSSSS | P12 5-10-5 (2′- | HTT |
| | ACAGAGGGA | | *SC*ST*SC*SC*SC*SC*SA | | SRSSRRRR | OMe-DNA-2′- | rs2530595 |
| | | | *SC*RA*SG*SmA*RmG*R | | | OMe) 2-2-11-2- | (C/T) |
| | | | mG*RmG*RmA | | | 2 (PS/PO) | |
| | | | | | | Gapmer with Rp | |
| | | | | | | wings | |
| WV-2675 | GGGUUCTCCCC | 2051 | mG*SmGmGmUmU*SC*ST | 2403 | SOOOSSSSSSS | P12 analogue of | HTT |
| | ACAGAGGGA | | *SC*SC*SC*SC*SA*SC*RA | | SRSSOOOS | WV-2595 with | rs2530595 |
| | | | *SG*SmAmGmGmG*SmA | | | G:U mismatch at | (C/T) |
| | | | | | | position 5 | |
| WV-2676 | GGCACAAGGGC | 2052 | mG*RmGmCmAmC*SA*SA | 2404 | ROOOSSSSSSS | WV-1092 | rs362307 |
| | ACAGACUUC | | *SG*SG*SG*SC*SA*SC*S | | SSSSOOOS | analogue for | Human HTT |
| | | | A*SG*SmAmCmUmU*SmC | | | CMC | |
| WV-2682 | GGCACAAGGGC | 2053 | mG*SmGmCmAmC*RA*SA | 2405 | SOOORSSSSSS | WV-1092 | rs362307 |
| | ACAGACUUC | | *SG*SG*SG*SC*SA*SC*S | | SSSSOOOS | analogue for | Human HTT |
| | | | A*SG*SmAmCmUmU*SmC | | | CMC | |
| WV-2683 | GGCACAAGGGC | 2054 | mG*SmGmCmAmC*SA*RA | 2406 | SOOOSRSSSSS | WV-1092 | rs362307 |
| | ACAGACUUC | | *SG*SG*SG*SC*SA*SC*S | | SSSSOOOS | analogue for | Human HTT |
| | | | A*SG*SmAmCmUmU*SmC | | | CMC | |
| WV-2684 | GGCACAAGGGC | 2055 | mG*SmGmCmAmC*SA*SA | 2407 | SOOOSSRSSSS | WV-1092 | rs362307 |
| | ACAGACUUC | | *RG*SG*SG*SC*SA*SC*S | | SSSSOOOS | analogue for | Human HTT |
| | | | A*SG*SmAmCmUmU*SmC | | | CMC | |
| WV-2685 | GGCACAAGGGC | 2056 | mG*SmGmCmAmC*SA*SA | 2408 | SOOOSSSRSSS | WV-1092 | rs362307 |
| | ACAGACUUC | | *SG*RG*SG*SC*SA*SC*S | | SSSSOOOS | analogue for | Human HTT |
| | | | A*SG*SmAmCmUmU*SmC | | | CMC | |
| WV-2686 | GGCACAAGGGC | 2057 | mG*SmGmCmAmC*SA*SA | 2409 | SOOOSSSSRSS | WV-1092 | rs362307 |
| | ACAGACUUC | | *SG*SG*RG*SC*SA*SC*S | | SSSSOOOS | analogue for | Human HTT |
| | | | A*SG*SmAmCmUmU*SmC | | | CMC | |
| WV-2687 | GGCACAAGGGC | 2058 | mG*SmGmCmAmC*SA*SA | 2410 | SOOOSSSSSRS | WV-1092 | rs362307 |
| | ACAGACUUC | | *SG*SG*SG*RC*SA*SC*S | | SSSSOOOS | analogue for | Human HTT |
| | | | A*SG*SmAmCmUmU*SmC | | | CMC | |
| WV-2688 | GGCACAAGGGC | 2059 | mG*SmGmCmAmC*SA*SA | 2411 | SOOOSSSSSSR | WV-1092 | rs362307 |
| | ACAGACUUC | | *SG*SG*SG*SC*RA*SC*S | | SSSSOOOS | analogue for | Human HTT |
| | | | A*SG*SmAmCmUmU*SmC | | | CMC | |
| WV-2689 | GGCACAAGGGC | 2060 | mG*SmGmCmAmC*SA*SA | 2412 | SOOOSSSSSSS | WV-1092 | rs362307 |
| | ACAGACUUC | | *SG*SG*SG*SC*SA*RC*S | | RSSSOOOS | analogue for | Human HTT |
| | | | A*SG*SmAmCmUmU*SmC | | | CMC | |
| WV-2690 | GGCACAAGGGC | 2061 | mG*SmGmCmAmC*SA*SA | 2413 | SOOOSSSSSSS | WV-1092 | rs362307 |
| | ACAGACUUC | | *SG*SG*SG*SC*SA*SC*S | | SSRSOOOS | analogue for | Human HTT |
| | | | A*RG*SmAmCmUmU*SmC | | | CMC | |
| WV-2691 | GGCACAAGGGC | 2062 | mG*SmGmCmAmC*SA*SA | 2414 | SOOOSSSSSSS | WV-1092 | rs362307 |
| | ACAGACUUC | | *SG*SG*SG*SC*SA*SC*S | | SSSROOOS | analogue for | Human HTT |
| | | | A*SG*RmAmCmUmU*SmC | | | CMC | |
| WV-2692 | GGCACAAGGGC | 2063 | mG*SmGmCmAmC*SA*SA | 2415 | SOOOSSSSSSS | WV-1092 | rs362307 |
| | ACAGACUUC | | *SG*SG*SG*SC*SA*SC*S | | SSSSOOOR | analogue for | Human HTT |
| | | | A*SG*SmAmCmUmU*RmC | | | CMC | |
| WV-2728 | GGCAC | | mG*SmGmCmAmC | | SOOO | WV-1092 | rs362307 |
| | | | | | | fragment for | Human HTT |
| | | | | | | CMC | |
| WV-2729 | GGCAC | | mG*RmGmCmAmC | | ROOO | WV-1092 | rs362307 |
| | | | | | | fragment for | Human HTT |
| | | | | | | CMC | |
| WV-2730 | ACUUC | | mAmCmUmU*SmC | | OOOS | WV-1092 | rs362307 |
| | | | | | | fragment for | Human HTT |
| | | | | | | CMC | |
| WV-2731 | ACUUC | | mAmCmUmU*RmC | | OOOR | WV-1092 | rs362307 |
| | | | | | | fragment for | Human HTT |
| | | | | | | CMC | |
| WV-2732 | GGCACAAGGGC | 2064 | mG*SmGmCmAmC*SA*SA | 2416 | SOOOSSSSSSR | WV-1092 for | rs362307 |
| | ACAGACUUC | | *SG*SG*SG*SC*RA*SC*R | | SRSSOOOS | CM | Human HTT |
| | | | A*SG*SmAmCmUmU*SmC | | | | |

### Abbreviations:

2\′: 2′
3\′: 3′
5\′: 5′
307: SNP rs362307
C6: C6 amino linker

### F, f: 2′-F

Htt, HTT: Huntingtin gene or Huntington's Disease
Lauric, Myristic, Palmitic, Stearic, Oleic, Linoleic, alpha-Linolenic, gamma-Linolenic, DHA, Turbinaric, Dilinoleic: Lauric acid, Myristic acid, Palmitic acid, Stearic acid, Oleic acid, Linoleic acid, alpha-Linolenic acid, gamma-Linolenic acid, docosahexaenoic acid, Turbinaric acid, Dilinoleyl methanol, respectively.
muHtt or muHTT: mutant Huntingtin gene or gene product

### OMe: 2′-OMe

O, PO: phoshodiester (phosphate)

### *, PS: Phosphorothioate

R, Rp: Phosphorothioate in Rp conformation
S, Sp: Phosphorothioate in Sp conformation
X: Phosphorothioate, stereorandom

In some embodiments, a composition or method described herein can pertain to any biologically active agent described herein, which can target any gene described herein, and any lipid described herein.

In some embodiments, a composition or method described herein can pertain to any biologically active agent described herein and any lipid described herein, for the treatment of any disease described herein.

In some embodiments, a composition or method described herein can pertain to any biologically active agent described herein and any lipid described herein.

Efficacy of Composition for Delivery of a Biologically Active Agent

In some embodiments, a composition for delivery of a biologically active agent is capable of performing two different functions: (a) delivering the biologically active agent (e.g., to particular targeted cells or tissues); and (b) allowing (e.g., not preventing or interfering with) the function of the biologically active agent. In some embodiments, a lipid increase the efficacy, activity, stability, bio-availability, tissue targeting, and/or biological half-life of a biologically active agent.

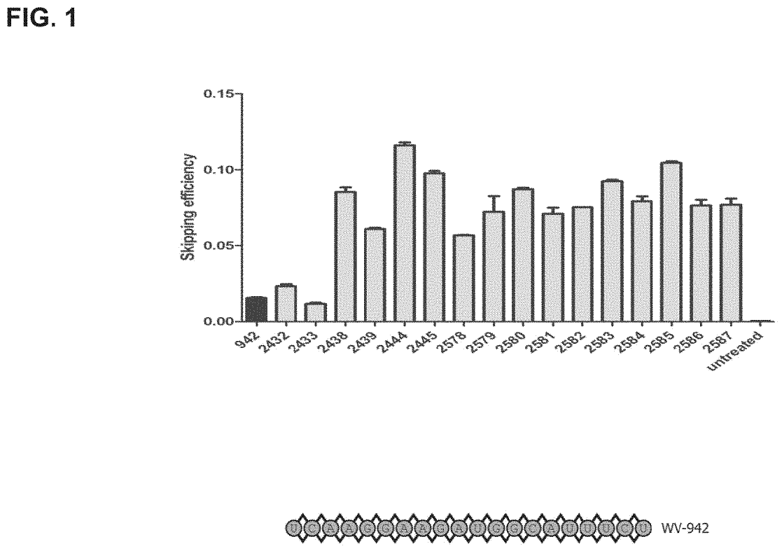

As shown in FIG. 1, certain example lipids for use in preparation of a composition for delivery of a biologically active agent allow (e.g., do not prevent or interfere with) the function of the biologically active agent. Non-limiting example lipids include: lauric acid, myristic acid, palmitic acid, stearic acid, oleic acid, linoleic acid, alpha-linolenic acid, gamma-linolenic acid, docosahexaenoic acid (DHA or cis-DHA), turbinaric acid and dilinoleyl.

A biologically active agent, oligonucleotide WV-942, was tested for its biological activity in human DMD (Duchenne muscular dystrophy) myoblasts. In the absence of exon 51 skipping, the protein is severely truncated due to a frameshift mutation, leading to a premature stop codon. Oligonucleotide WV-942, which has a sequence and chemical identical to Drisapersen, also known as Kyndrisa, PRO051 and GSK2402968, is intended to allow skipping of exon 51, thus allowing production of a frame-corrected dystrophin transcript which lacks exon 51. Experimental details are provided in Example 2.

In this experiment, the myoblast cells were treated with naked WV-942 (not conjugated to any lipid), or WV-942 conjugated to any one of several lipids: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

TABLE 1
Lipids conjugated to biologically active agent,
oligonucleotide WV-942.

| Oligonucleotide | Conjugated Acid |
|---|---|
| WV-942 | — |
| WV-2578 | Lauric acid |
| WV-2579 | Myristic Acid |
| WV-2580 | Palmitic acid |
| WV-2581 | Stearic acid |
| WV-2582 | Oleic acid |
| WV-2583 | Linoleic acid |
| WV-2584 | Alpha-Linolenic acid |
| WV-2585 | Gamma-Linolenic acid |
| WV-2586 | cis-DHA |
| WV-2587 | Turbinaric acid |
| WV-2588 | Dilinoleyl |

These results show that preparing a composition comprising a biologically active agent, WV-942, and any of several lipids (lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl) did not prevent biological activity of the agent; in fact, in several cases, in the addition of a lipid, biological activity was increased several-fold.

Among other things, the present disclosure encompasses the recognition that lipids can surprisingly enable and/or promote delivery of biologically active agents to their target location(s) (e.g., cells, tissues, organs, etc.) In some embodiments, lipids can be utilized to effectively improve delivery of biologically active agents to their target location(s) in a subject, e.g., in a mammal or human subject, etc. The present disclosure particularly documents the surprising achievement of efficient and/or effective delivery of biologically active agent(s) into cells (i.e., to intracellular location(s)). The present disclosure also shows the additional surprising finding that lipids can improve the pharmacokinetics (e.g., optimized half-life) of an administered biologically active agent. The present disclosure also documents the additional surprising finding that lipids can be utilized to improve immune characteristics of delivered biologically active agents, e.g., by antagonizing an immune response mediated by TLR9.

Targeting of Particular Cells or Tissues

In some embodiments, a composition for delivery of a biologically active agent is capable of targeting the biologically active agent to particular cells or tissues, as desired.

In some embodiments, a composition for delivery of a biologically active agent is capable of targeting the biologically active agent to a muscle cell or tissue. In some embodiments, the present disclosure pertains to compositions and methods related to delivery of biologically active agents, wherein the compositions comprise a biologically active agent a lipid. In various embodiments to a muscle cell or tissue, the lipid is selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

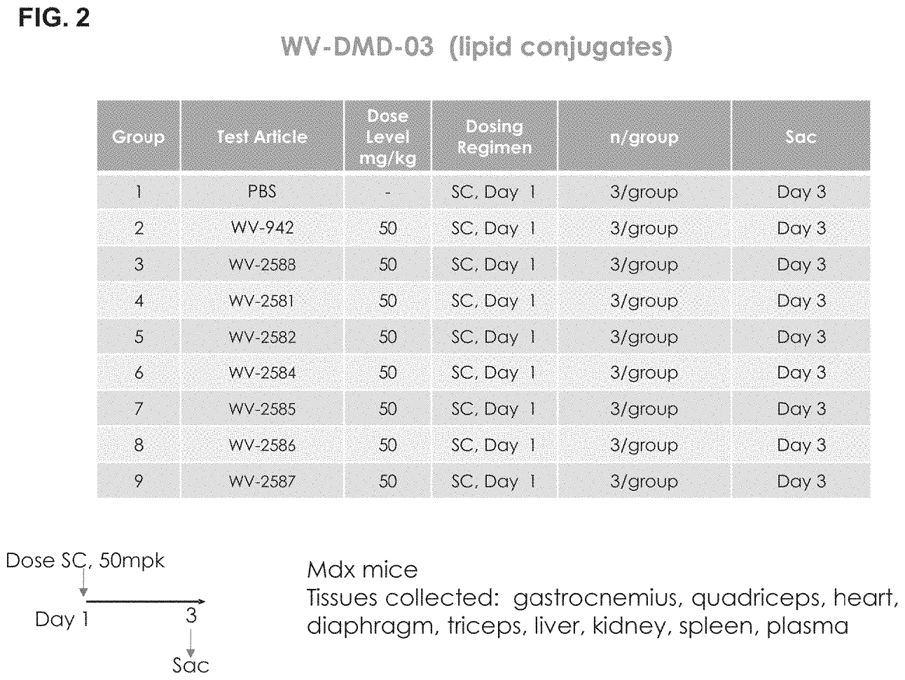

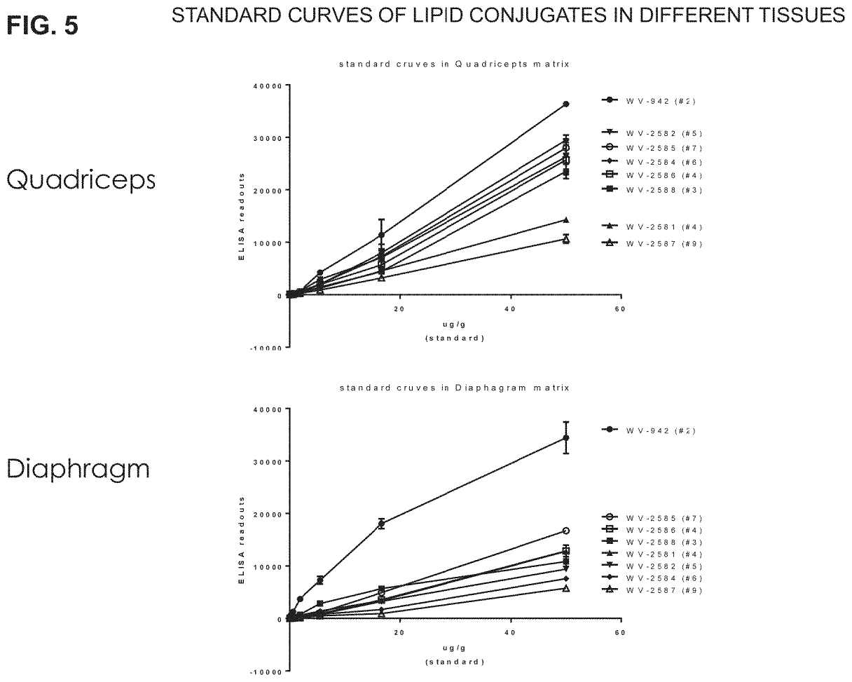

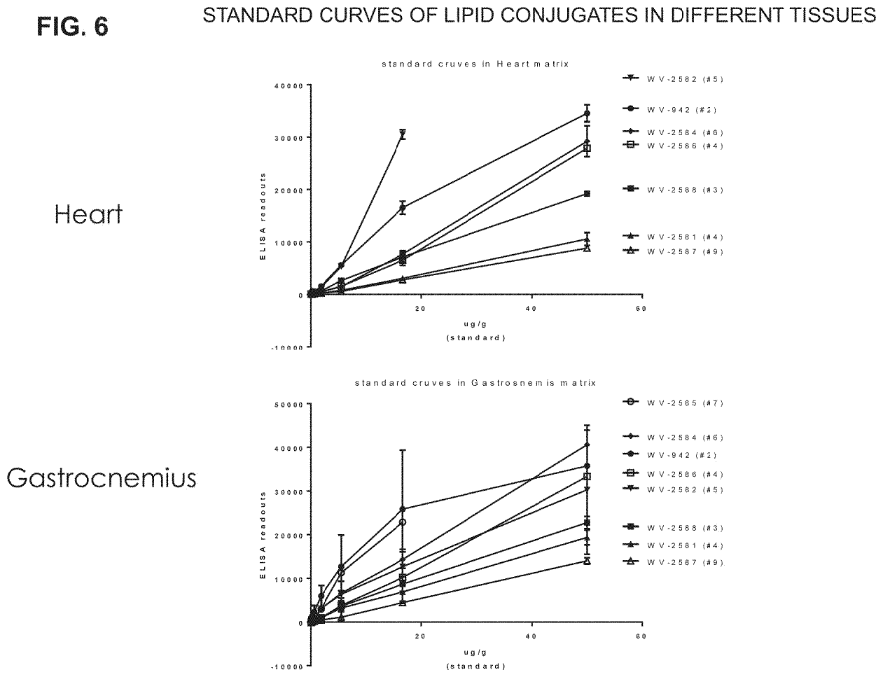

As shown in FIGS. 2 to 6, example compositions were prepared comprising a biologically active agent (WV-942) and a lipid, and these compositions were capable of delivering the biologically active agent to target cells and tissues, e.g., muscle cells and tissues. The example lipids used include stearic acid, oleic acid, alpha-linolenic acid, gamma-linolenic acids, cis-DHA, turbinaric acid and dilinoleyl acid. In these figures, “TBD” indicates that the particular composition was effective for delivery, but the numerical results were outside the standard range, and thus the final results remain to be determined; however, the compositions indicated as “TBD” in the Figures were efficacious at delivery of a biologically active agent.

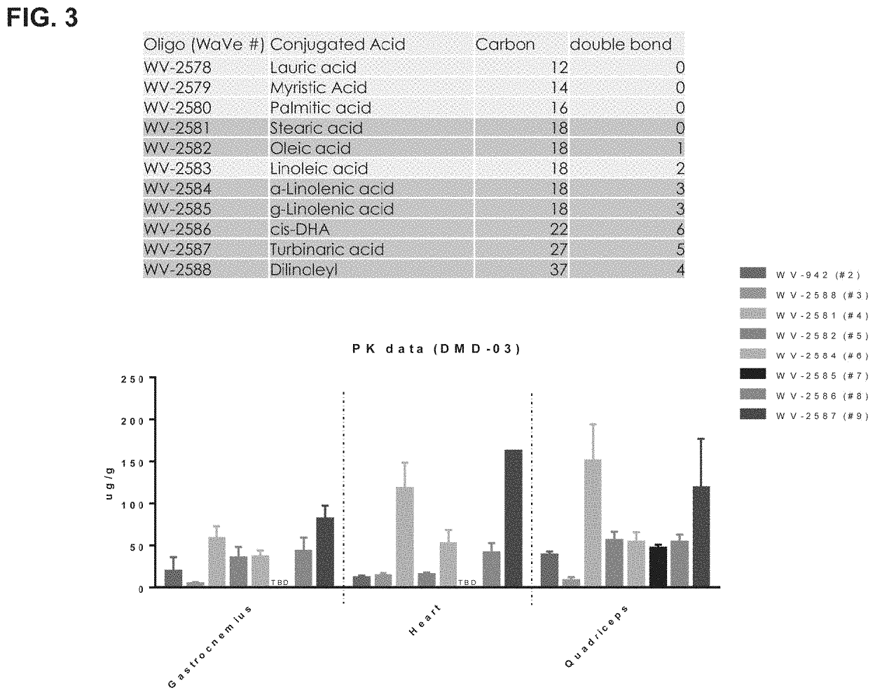

As shown in FIG. 3: A composition comprising a biologically active agent and any of: stearic acid, oleic acid, alpha-linolenic acid, gamma-linolenic acid, cis-DHA or turbinaric acid, was able to deliver the biologically active agent to gastrocnemius muscle tissue.

A composition comprising a biologically active agent and any of: stearic acid, alpha-linolenic, gamma-linolenic, cis-DHA, or turbinaric acid, was able to deliver the biologically active agent to heart muscle tissue.

A composition comprising a biologically active agent and any of: stearic acid, oleic acid, alpha-linolenic acid, gamma-linolenic acid, cis-DHA or turbinaric acid, was able to deliver the biologically active agent to quadriceps muscle tissue.

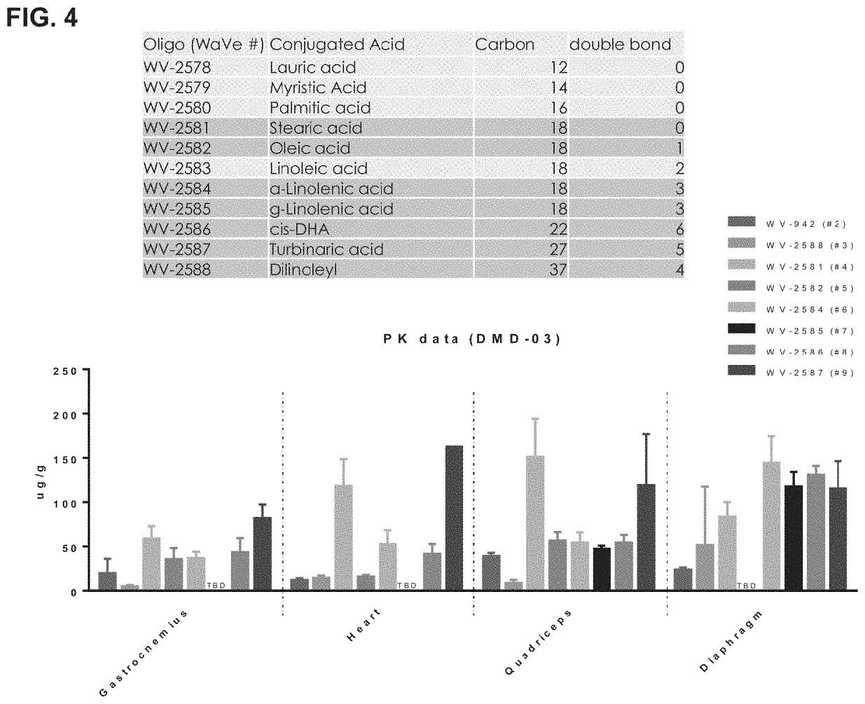

As shown in FIG. 4: A composition comprising a biologically active agent and any of: stearic, oleic, alpha-linolenic, gamma-linolenic, cis-DHA, or turbinaric acid was able to deliver the biologically active agent to the gastrocnemius muscle tissue.

A composition comprising a biologically active agent and any of: stearic acid, alpha-linolenic, gamma-linolenic, cis-DHA, or turbinaric acid was able to deliver the biologically active agent to heart muscle tissue.

A composition comprising a biologically active agent and any of: dilinoleyl, stearic acid, oleic acid, alpha-linolenic, gamma-linolenic, cis-DHA or turbinaric acid was able to delivery the biologically active agent to the thoracic diaphragm muscle tissue.

In some embodiments, conjugation of a lipid to an oligonucleotide improves at least one characteristic of the oligonucleotide. In some embodiments, the characteristic is increased activity (e.g., increased ability to induce desirable skipping of a deleterious exon), decreased toxicity, or improved distribution to a tissue. In some embodiments, the tissue is muscle tissue. In some embodiments, the tissue is skeletal muscle, gastrocnemius, triceps, heart or diaphragm.

The ability of conjugation of lipids to improve the distribution of oligonucleotides is shown in FIGS. 31A to 31D.

The tested oligonucleotides (WV-3473, WV-3545, WV-3546 and WV-942) were intravenous injected via tail vein in male C57BL/10ScSndmdmdx mice (4-5 weeks old), at 10 mg/kg or 30 mg/kg. Tissues were harvested on Day 2, 7 and 14 after injection, fresh-frozen in liquid nitrogen and stored in −80° C. until analysis.

Hybrid-ELISA is used to quantify the ASO levels in tissue using test article serial dilution as standard curve: Maleic anhydride activated 96 well plate (Pierce 15110) was coated 50 μL of capture probe at 500 nM in 2.5% NaHCO₃(Gibco, 25080-094) for 2 hours at 37° C. The plate then washed 3 times with PBST (PBS+0.1% Tween-20), blocked with 5% fat free milk-PBST at 37° C. for 1 hour. Test article ASO was serial diluted into matrix. This standard together with original samples were diluted with lysis buffer (4 M Guanidine; 0.33% N-Lauryl Sarcosine; 25 mM Sodium Citrate; 10 mM DTT) so that ASO amount in all samples is less than 100 ng/ml. 20 μL of diluted samples were mixed with 180 μL of 333 nM detection probe diluted in PBST, then denatured in PCR machine (65° C., 10 min, 95° C., 15 min, 4° C. ∞). 50 μL of denatured samples were distributed in blocked ELISA plate in triplicates, and incubated overnight at 4° C. After 3 washes of PBST, 1:2000 streptavidin-AP in PBST was added, 50 μL per well and incubated at room temperature for 1 hour. After extensive wash with PBST, 100 μL of AttoPhos (Promega S1000) was added, incubated at room temperature in dark for 10 min and read on plate reader (Molecular Device, M5) fluorescence channel: Ex 435 nm, Em 555 nm. The ASO in samples were calculated according to standard curve by 4-parameter regression.

In some embodiments, for example as shown in certain Figures, WV-3473 has no detectable level in Gastrocnemius, Triceps, Heart or Diaphragm, in contrast to WV-942. The stability of WV-3473 is good in both plasma and tissue homogenates. In some embodiments, for example as demonstrated in certain Figures, lipid-conjugation of WV-3473 improves the muscle distribution of WV-3473, often without impacting removal of oligonucleotides from a system.

Thus: A composition comprising a lipid, selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, and a biologically active agent is capable of delivering the biologically active agent to extra-hepatic cells and tissues, e.g., muscle cells and tissues.

### Pharmacokinetics

In some embodiments, one or more characteristics of pharmacokinetics of a drug (e.g., a drug comprising a biologically active agent, including but not limited to an oligonucleotide) can be optimized by conjugation with a lipid.

In some embodiments, pharmacokinetics pertains to analysis of drug metabolism, including analysis of the fate of a drug from the moment of administration to the time of elimination from the body. Pharmacokinetics can encompass, as a non-limiting example, the quantification of a drug or its metabolite in a particular tissue (e.g., blood or muscle) over time.

Various pharmacokinetic characteristics include, but are not limited to: Cₘₐₓ, peak plasma concentration of a drug after administration; tₘₐₓ, time to reach Cₘₐₓ; Cₘᵢₙ, lowest (trough) concentration that a drug reaches before the next dose is administered; Elimination half-life, the time required for the concentration of the drug to reach half of its original value; Elimination rate constant, rate at which a drug is removed from the body; Area under the curve, integral of the concentration-time curve (after a single dose or in steady state); and Clearance, volume of plasma cleared of the drug per unit time.

Various pharmacokinetic characteristics of a particular drug can be influenced by any one or more of: total dose, number of dosages, rate of administration, method of administration, administration vehicle, bodily site of administration, etc. Various characteristics of pharmacokinetics and various influences on them are known in the art.

The present disclosure, among other things, shows that one or more characteristics of pharmacokinetics of a drug comprising a biologically active agent (e.g., an oligonucleotide) can be affected, improved and/or optimized by conjugation of the agent to a lipid.

In general, it is noted that optimization of a pharmacokinetic characteristic such as half-life can be distinguished from maximization. In some embodiments, in general, it may be desirable for a particular drug to have a half-life sufficient to allow performance of its desired function, but short enough to minimize off-target effects and other toxicity. Thus, in some embodiments, an optimized half-life is long enough to allow activity while minimizing toxicity; a prolonged or maximized half-life may be undesirable.

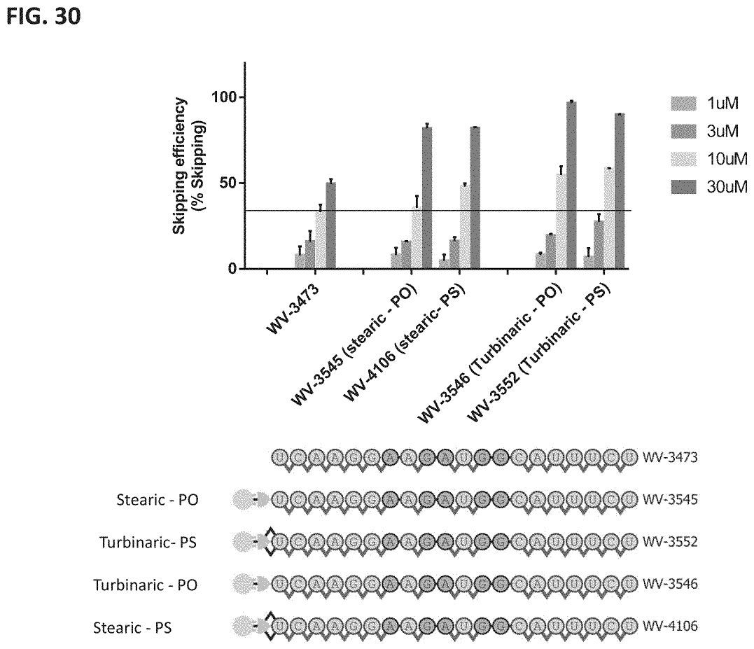

The present disclosure shows that conjugation with a lipid can improve the half-life of a biologically active agent. FIGS. 31A to 31D show the distribution of oligonucleotides (including some conjugated to a lipid) in various muscle tissues. Muscles tested include: gastrocnemius (FIG. 31A); triceps (FIG. 31B); heart (FIG. 31C); and diaphragm (FIG. 31D). Control oligonucleotide WV-942 is equivalent to Drisapersen, which has an undesirably long half-life, which can contribute to toxicity. Test oligonucleotide WV-3473 was administered to animals naked, or conjugated to a lipid (stearic acid, WV-3545; or turbinaric acid, WV-3546). In some of the assays, conjugation to a lipid improved half-life of the oligonucleotide, without extending it to an undesirably long length. See, for example, FIG. 30C, which shows that conjugation of either stearic acid or turbinaric acid to the oligonucleotide, which administered at 30 mg/kg, increased distribution to heart tissue, particularly at Day 3 and 8, but did not increase it to the level of WV-942, which is known to have an undesirably long half-life.

### Lipids, Immunostimulation and TLR9

In some embodiments, the present disclosure encompasses the surprising finding that lipid conjugation can effectively antagonize an immune response, e.g., that mediated by TLR9.

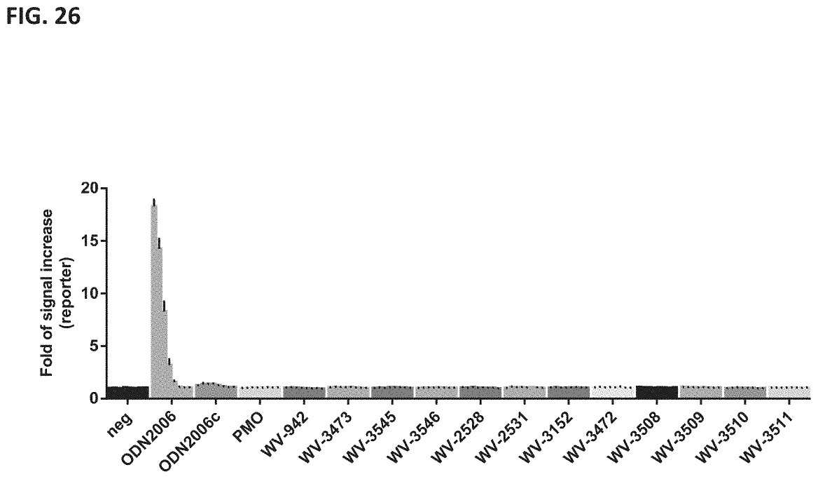

In some embodiments, example data demonstrated that many of provided oligonucleotides do not mediate an immune response, as determined by a lack of agonism of hTLR9; see FIG. 26. Among other things, the present disclosure demonstrates that oligonucleotides conjugated to lipids surprisingly counteracted hTLR9 agonism, for example, that mediated by control oligonucleotide ODN2006 (e.g., conjugation of lipids to oligonucleotides antagonizes hTLR9 activity mediated by ODN2006); for examples, see FIGS. 27 and 28, WV-3545 and WV-3546 (which are oligonucleotides to the target Dystrophin). Other oligonucleotides comprising lipid moieties were also tested and were shown to have greatly enhanced ability to antagonize hTLR9 activity. For example, WV-2824 and WV-2830, conjugates of Malat1-targeting WV-2735 with stearic acid (WV-2824) and turbinaric acid (WV-2830), respectively, also demonstrated greatly enhanced ability to antagonize hTLR9 activity mediated by ODN2006. Among other things, these experiments show that conjugation of lipids, such as stearic acid, turbinaric acid, etc., with oligonucleotides can greatly increase hTLR9 antagonist activity.

TLR9 is Toll-Like Receptor 9, also known as CD289; RefSeq (mRNA) NM_017442; RefSeq (protein) NP_059138. hTLR9 is human TLR9.

Microarray

In some embodiments, the present disclosure pertains to a microarray comprising a collection of one or more chirally controlled oligonucleotides. In some embodiments, a microarray comprises multiple spatially defined regions, wherein each region comprises a chirally controlled oligonucleotide composition. In some embodiments, a microarray comprises a solid phase support having a surface (e.g., a planar surface), which carries a collection of types of chirally controlled oligonucleotides, wherein each type is immobilized to a spatially defined region or site on the surface, wherein the region or site of one type does not overlap with the region or site of any other type. In some embodiments, oligonucleotides of different types can differ in base sequence (e.g., comprising overlapping or tiled sequences), pattern of backbone modification, pattern of stereochemistry, and/or conjugation with any of a variety of lipids or other moieties.

In some embodiments, a microarray can be used in for a variety of purposes. In some embodiments, a microarray can be used to test the comparative qualities of various aspects (e.g., base sequence, pattern of backbone modification, pattern of stereochemistry, and/or conjugation with a lipid or other moiety) of different types of oligonucleotides. In some embodiments, different types of oligonucleotides can be tested for their ability to bind to particular target proteins or nucleic acids, ability to mediate RNA interference, ability to alter exon skipping (e.g., increasing desired skipping or decrease undesired skipping), ability to mediate knockdown via a RNAseH mechanism, resistance to nucleases, immunogenicity, TLR9 agonism and/or antagonism, etc. Using a microarray, in some embodiments multiple types of oligonucleotides can be exposed to the same experimental fluids and test conditions, and thus multiple types of oligonucleotides can be simultaneously tested. As a non-limiting example, in order to test resistance of multiple types of oligonucleotides to a nuclease, various types of oligonucleotides can be immobilized in different regions on a microarray, which is then subjected to a fluid comprising a nuclease. The relative resistance of various oligonucleotide types to nuclease degradation can be readily determined. As another non-limiting example, in order to test the relative ability of different oligonucleotide types to mediate RNA interference, different types can be immobilized in different regions of a microarray, which is then treated with a fluid comprising the various components required for testing RNA interference (mRNA target, RNA interference complex, buffer, detection moieties, etc.); the relative ability of different oligonucleotide types to mediate RNA interference can thus be readily determined. Any method known in the art can be used to determine the relative activity of interest of each oligonucleotide type, including but not limited to: detection or use of fluorescent, chemiluminescent, chemical, or radioactive markers, labels or dyes.

Methods of producing microarrays, which may also be referred to as gene chips, gene arrays, or nucleic acid chips, or by other terms, are known in the art and can be used in accordance with the present disclosure. In some embodiments, microarrays are produced by robots. In some embodiments, a method of producing a microarray comprises a step of separately producing various oligonucleotide types and then a step of depositing various oligonucleotide types on designated regions of a microarray. In some embodiments, for each oligonucleotide type, a fine needle or pin is dipped into a well comprising that type, and the needle or pin used to deposit each type onto a microarray. In some embodiments, a method of producing a microarray comprises a step of polymerizing various oligonucleotide types on designated regions of a microarray. Various methods of making microarrays are known in the art. Also known in the art are various materials from which a microarray can be constructed (glass, plastic, polystyrene, etc.).

In some embodiments, a microarray comprises a collection of beads, wherein each bead is physically discrete from each other, but wherein various beads are mixed or combined in a single sample. Each bead or type of bead (e.g., a particular type of bead to which is immobilized an oligonucleotide type) can comprise, for example, a specific ratio of two or more quantification agents (e.g., dyes), such that beads can be differentiated and the relative activity of each oligonucleotide type can be determined.

In some embodiments, the present disclosure pertains to a collection of types of oligonucleotide types, wherein each type is defined by any one or more of: base sequence, pattern of backbone modification, pattern of stereochemistry, and/or conjugation with a lipid or other moiety. The collection (e.g., a microarray) can be used to test the relative qualities or abilities or various oligonucleotide types.

Additional Optional Components of the Composition

In some embodiments, the present disclosure pertains to compositions and methods related to delivery of biologically active agents, wherein the compositions comprise a biologically active agent and a lipid comprising a C₁₀-C₄₀ linear, saturated or partially unsaturated, aliphatic chain. In some embodiments, the present disclosure pertains to compositions and methods related to delivery of biologically active agents, wherein the compositions comprise a biologically active agent and a lipid comprising a C₁₀-C₄₀ linear, saturated or partially unsaturated, aliphatic chain, optionally substituted with one or more C₁₋₄ aliphatic group.

In some embodiments, the present disclosure pertains to compositions and methods related to delivery of biologically active agents, wherein the compositions comprise a biologically active agent and a lipid. In various embodiments, the lipid is selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

In various embodiments, the composition for delivery of a biologically active agent furhter comprises an additional, optional, component. In various embodiments, the additional optional component is selected from: one or more additional lipids; a targeting compound or moiety; a 3′ end cap (in the example of a nucleic acid); and a carbonic anhydrase inhibitor.

In some embodiment, a composition comprises a lipid, a biologically active agent and any one or more additional components selected from: a polynucleotide, a dye, an intercalating agent (e.g. an acridine), a cross-linker (e.g. psoralene, or mitomycin C), a porphyrin (e.g., TPPC4, texaphyrin, or Sapphyrin), a polycyclic aromatic hydrocarbon (e.g., phenazine, or dihydrophenazine), an artificial endonuclease, a chelating agent, EDTA, an alkylating agent, a phosphate, an amino, a mercapto, a PEG (e.g., PEG-40K), MPEG, [MPEG]₂, a polyamino, an alkyl, a substituted alkyl, a radiolabeled marker, an enzyme, a hapten (e.g. biotin), a transport/absorption facilitator (e.g., aspirin, vitamin E, or folic acid), a synthetic ribonuclease, a protein, e.g., a glycoprotein, or peptide, e.g., a molecule having a specific affinity for a co-ligand, or antibody e.g., an antibody, a hormones, a hormone receptor, a non-peptidic species, a lipid, a lectin, a carbohydrate, a vitamin, a cofactor, a carbonic anhydrase inhibitor, or a drug.

In some embodiment, a composition comprises a lipid, a biologically active agent and any one or more additional components, wherein the one or more additional components comprises or consists of a carbonic anhydrase inhibitor. Carbonic anhydrases are a family of 16 members that regulate intracellular and extracellular pH. In some embodiments, the expression of the CA3 gene is strictly tissue-specific and present at high levels in skeletal muscle and much lower levels in cardiac and smooth muscle. In some embodiments, CA3 is insufficient in muscles of Myasthenia Gravis patients. In some embodiments, a proportion of carriers of Duchenne muscle dystrophy have a higher CA3 level than normal. In some embodiments, CA IV, the first membrane-associated isoform to be studied, is expressed in a wide variety of tissues including kidney, heart, lung, gall bladder, distal small intestine, colon, and skeletal muscle. In some embodiments, in human tissues CA XIV is expressed in heart, followed by brain, skeletal muscle, and liver; no signal was seen in lung or kidney. In some embodiments, a carbonic anhydrase inhibitor inhibits a CA3, CA IV, CA XIV and/or any one or more CA genes and/or gene products. A non-limiting example of a CA inhibitor, along with a linker for linking the CA inhibitor to a biologically active agent, is shown in FIG. 30.

### Additional Lipids

In some embodiments, the present disclosure pertains to compositions and methods related to a composition comprising a biologically active agent and a lipid comprising a C₁₀-C₄₀ linear, saturated or partially unsaturated, aliphatic chain. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group.

In some embodiments, the present disclosure pertains to compositions and methods related to a composition comprising: a biologically active agent; a lipid comprising a C₁₀-C₄₀ linear, saturated or partially unsaturated, aliphatic chain; and an additional lipid or lipids. In some embodiments, the present disclosure pertains to compositions and methods related to a composition comprising: a biologically active agent; a lipid comprising a C₁₀-C₄₀ linear, saturated or partially unsaturated, aliphatic chain, optionally substituted with one or more C₁₋₄ aliphatic group; and an additional lipid or lipids.

In various embodiments, the composition for delivery of a biologically active agent comprises: a biologically active agent; a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl; and an additional lipid or lipids.

In some embodiments, an additional lipid or lipids is selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. As a non-limiting example: In various embodiments, the composition for delivery of a biologically active agent comprises a biologically active agent, and two or more lipids selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

In various embodiments, the additional lipid or lipids can be selected from: an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; and a targeting lipid or other targeting component; and any other lipid described herein or reported in the art. In various embodiments, the additional lipids can comprise a combination of lipids, as a non-limiting example, an amino-lipid, a cationic lipid, a helper lipid and/or a PEG-lipid and/or a hydrophobic small molecule. Additional components that may be present in a lipid particle include bilayer stabilizing components such as polyamide oligomers (see, U.S. Pat. No. 6,320,017), peptides, proteins, detergents, lipid-derivatives, such as PEG coupled to phosphatidylethanolamine and PEG conjugated to ceramides (see, U.S. Pat. No. 5,885,613).

The types and ratios of various lipids (e.g., a lipid and an additional, optional lipid or lipids) in the composition can be modulated to perform any one or more of the following: improve cellular or tissue targeting; improve cellular uptake; improve endosomal escape; reduce liver toxicity; increase efficiency of delivery; increase tolerability; improve consistency in size of lipid nanoparticles; reduce aggregation of lipid nanoparticles; prevent charge-induced aggregation of lipid nanoparticles; improve chemical stability, improve half-life in circulation, and/or reduce degradation of the biologically active agent (e.g., by nucleases in the case of nucleic acids, or proteases in the case of proteins).

The composition for delivery of the present disclosure can be in the form of a lipid nanoparticle (LNP). As used herein, the term “lipid nanoparticles” includes liposomes irrespective of their lamellarity, shape or structure and lipoplexes as described for the introduction of pDNA into cells (PNAS, 1987, 84, 7413). These lipid nanoparticles can be complexed with biologically active agents and are useful as in vivo delivery vehicles.

Various lipids are described below, and/or reported in the art, including, as non-limiting examples: U.S. Pat. Nos. 9,315,437; 9,278,130; 9,254,327; 9,242,001; and 9,220,785; US patent applications: US 2009/0263407, US 2009/0285881, US 2010/0055168, US 2010/0055169, US 2010/0063135, US 2010/0076055, US 2010/0099738, and US 2010/0104629; Semple S. C. et al., Rational design of cationic lipids for siRNA delivery, Nature Biotechnology, published online 17 Jan. 2010; doi:10.1038/nbt.1602; and documents cited therein.

In some embodiments, an additional lipid or lipids comprises an amino lipid. As a non-limiting example, an amino lipid includes a lipid having at least one nitrogen atom incorporated in at least one fatty acid chain. This fatty acid chain may be an alkyl, alkenyl or alkynyl carbon chain. As non-limiting examples, lipids contain carbon chain lengths in the range from C10 to C20. The fatty acid portion of the amino-lipid can be incorporated through the use of suitable carbonyl compounds such as aldehydes (R—CHO) and ketones (R—CO—R). Through the use of asymmetrical ketones (R—CO—R′) corresponding unsymmetrical substituted lipids can be prepared. Likewise, through the use of carbonyl ethers, esters, carbamates and amides and suitable reducing agents the corresponding amino-lipids are accessible.

In some embodiments, an additional lipid or lipids comprises an amphipathic lipid. As a non-limiting example, an amphipathic lipid includes any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Additionally, such amphipathic lipids can be readily mixed with other lipids, such as triglycerides and sterols.

In some embodiments, an additional lipid or lipids comprises an anionic lipid. As non-limiting examples, an anionic lipid includes a compound selected from phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol and other anionic modifying groups joined to neutral lipids.

In some embodiments, an additional lipid or lipids comprises an apolipoprotein, also known as a lipoprotein, or a fragment thereof. As non-limiting examples, apolipoproteins include ApoA-I, ApaA-II, ApoA-IV, ApaA-V and ApoE, and active polymorphic forms, isoforms, variants and mutants as well as fragments or truncated forms thereof. In certain embodiments, the apolipoprotein is a thiol-containing apolipoprotein, which contains at least one cysteine residue. The most common thiol-containing apolipoproteins are ApoA-I Milano (ApoA-IM) and ApoA-I Paris (ApoA-Ip), which contain one cysteine residue (Jia et al., 2002, Biochem. Biophys. Res. Comm. 297: 206-13; Bielicki and Oda, 2002, Biochemistry 41: 2089-96). ApoA-II, ApoE2 and ApoE3 are also thiol-containing apolipoproteins. Isolated ApoE and/or active fragments and polypeptide analogues thereof, including recombinantly produced forms thereof, are described in U.S. Pat. Nos. 5,672,685; 5,525,472; 5,473,039; 5,182,364; 5,177,189; 5,168,045; and 5,116,739. ApoE3 is disclosed in Weisgraber, et al., “Human E apoprotein heterogeneity: cysteine-arginine interchanges in the amino acid sequence of the apo-E isoforms,” J. Biol. Chem. (1981) 256: 9077-9083; and Rall, et al., “Structural basis for receptor binding heterogeneity of apolipoprotein E from type III hyperlipoproteinemic subjects,” Proc. Nat. Acad. Sci. (1982) 79: 4696-4700. See also GenBank accession number K00396. In certain embodiments, the apolipoprotein can be in its mature form, in its (preproapolipoprotein form or in its proapolipoprotein form, Homo- and heterodimers (where feasible) of pro- and mature ApoA-I (Duverger et al., 1996, Arterioscler. Thromb. Vase. Biol. 16(12):1424-29), ApoA-I Milano (Klon et al., 2000, Biophys. J. 79:(3)1679-87; Franceschini et al., 1985, J. Biol. Chem. 260: 1632-35), ApoA-I Paris (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), and ApoE (McLean et al., 1983, J. Biol. Chem. 258(14):8993-9000) can also be utilized.

In some embodiments, an additional lipid or lipids comprises a cationic lipid. In a non-limiting example, a cationic lipid is an amino lipid. As a non-limiting example, a cationic lipid includes a lipid comprising a quaternary amine with a nitrogen atom having four organic substituents. Non-limiting examples of cationic lipids comprising a quaternary amine include N-(2,3-dioleyloxy)propyl)-N,N,N-trimethylammonium chloride (“DOTAP”), N,N-Distearyl-N,N-dimethylammonium bromide (“DDBA”), 1-methyl-4-(cis-9-dioleyl)-methylpyridinium-chloride (“SAINT-solid”)N-(2,3-dioleyloxy)propyl)-N,N,N-triethylammonium chloride (“DOTMA”), N,N-dioleyl-N,N-dimethylammonium chloride (“DODAC”), (1,2-dimyristyloxyprop-3-yl)-N,N-dimethyl-N-hydroxyethyl ammonium bromide (“DIMRIE”), N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3beta-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioetadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE, (comprising DOSPA and DOPE, available from GIBCO/BRL). As a non-limiting example, a cationic lipid can have certain design features including a head group, one or more hydrophobic tails, and a linker between the head group and the one or more tails. The head group can include an amine; for example an amine having a desired pKa. The pKa can be influenced by the structure of the lipid, particularly the nature of head group; e.g., the presence, absence, and location of functional groups such as anionic functional groups, hydrogen bond donor functional groups, hydrogen bond acceptor groups, hydrophobic groups (e.g., aliphatic groups), hydrophilic groups (e.g., hydroxyl or methoxy), or aryl groups. The head group amine can be a cationic amine; a primary, secondary, or tertiary amine; the head group can include one amine group (monoamine), two amine groups (diamine), three amine groups (triamine), or a larger number of amine groups, as in an oligoamine or polyamine. The head group can include a functional group that is less strongly basic than an amine, such as, for example, an imidazole, a pyridine, or a guanidinium group. The head group can be zwitterionic. Other head groups are suitable as well. The one or more hydrophobic tails can include two hydrophobic chains, which may be the same or different. The tails can be aliphatic; for example, they can be composed of carbon and hydrogen, either saturated or unsaturated but without aromatic rings. The tails can be fatty acid tails; some such groups include octanyl, nonanyl, decyl, lauryl, myristyl, palmityl, stearyl, alpha-linoleyl, stearidonyl, linoleyl, gamma-linolenyl, arachadonyl, oleyl, and others. Other hydrophobic tails are suitable as well. The linker can include, for example, a glyceride linker, an acyclic glyceride analog linker, or a cyclic linker (including a spiro linker, a bicyclic linker, and a polycyclic linker). The linker can include functional groups such as an ether, an ester, a phosphate, a phosphonate, a phosphorothioate, a sulfonate, a disulfide, an acetal, a ketal, an imine, a hydrazone, or an oxime. Other linkers and functional groups are suitable as well.

In some embodiments, an additional lipid or lipids comprises a cloaking component. As a non-limiting example, a cloaking component can include a fusion delaying component. As non-limiting examples, the cloaking component can include an ATTA-lipid conjugate or a PEG-lipid conjugate, and can simply exchange out of the lipid particle membrane over time. By the time the lipid particle is suitably distributed in the body, it has lost sufficient cloaking agent so as to be fusogenic.

In some embodiments, an additional lipid or lipids comprises a helper lipid. Non-limiting examples of helper lipids include 1,2-distearoyl-sa-glycero-3-phosphocholine (“DSPC”), 1,2-dipalmitoyl-sn-glycero-3-phosphocholine (“DPPC”), or any related phosphatidylcholine such as natural sphingomyelin (“SM”) and synthetic derivatives thereof such as 1-oleoyl-2-cholesteryl-hemisuccinoyl-sn-glycero-3-phosphocholine (“OChemsPC”). Other helper lipids include 1,2-dileoyl-sn-3-phosphoethanolamine (“DOPE”), 1,2-diphytanoyl-sn-glycero-3-phosphoethanol-amine (“ME 16:0 PE”).

In some embodiments, an additional lipid or lipids comprises a lipopeptide compound. As a non-limiting example, a lipopeptide compound includes a central peptide and having lipophilic groups attached at each terminus, and salts and uses thereof. The lipophilic groups can be derived from a naturally-occurring lipid, or can be a C(1-22)alkyl, C(6-12)cycloalkyl, C(6-12)cycloalkyl-alkyl, C(3-18)alkenyl, C(3-18)alkynyl, C(1-5)alkoxy-C(1-5)alkyl, or a sphinganine, or (2R,3R)-2-amino-1,3-octadecanediol, icosasphinganine, sphingosine, phytosphingosine, and cis-4-sphingenine.

In some embodiments, an additional lipid or lipids comprises a PEG-lipid. As a non-limiting example, a PEG-lipid includes an uncharged lipid modified with one or more hydrophilic polymers, e.g. polyethylene glycol (herein also referred to as “PEG-lipids”) to stabilize the lipid nanoparticle and to avoid aggregation. The polyethylene glycol (PEG) size can vary from approximately 1 to 5 approximately kDa. Depending on the relative amounts of these molecules in the formulation and the length of the hydrocarbon chain, the PEG-lipid can influence the pharmacokinetic characteristics, biodistribution, and efficacy of a formulation. PEG lipids having relatively short lipid hydrocarbon chains of about 14 carbons dissociate from the LNP in vivo in plasma with a half-life of less than 1 h. In contrast, a PEG lipid with a relatively long lipid hydrocarbon chain length of about 18 carbons circulates fully associated with the formulation for several days. Hence, in one embodiment, the PEG lipid comprises a lipid hydrocarbon chain of 12 to 20 carbon atoms, 14 to 18 carbon atoms, or 14 carbon atoms. Non-limiting examples of suitable PEG modified lipids include pegylated ceremide conjugates, pegylated distearoylphosphatidyl-ethanolamine (PEG-DSPE). Other compounds that can be used to stabilize lipid nanoparticles include gangliosides (GMt, GM3, etc.). As a non-limiting example, PEG lipids have a PEG size ranging from about 1 to about 2 KDa. Specific examples are methoxy-polyethyleneglycol-carbamoyl-dimyristyloxy-propylamine (PEG2000-c-DMA), and (alpha-(3′-(1,2-dimyristoyl-3-propanoxy)carboxamide-propyl]-.omega.-met-hoxy-polyoxyethylene (PEG2000-c-DOMG). In some embodiments, a PEG-lipid is polyethyleneglycol-dimyristoyl-phosphatidylethanolamine.

In some embodiments, an additional lipid or lipids comprises a hydrophobic small molecule. In a non-limiting example, a hydrophobic small molecule includes a compound with a molecular weight of about 300 to about 700 Da comprising 2 or more carbon- or heterocycles providing a rigid core structure. As a non-limiting example, a hydrophobic small molecule is selected from the group of sterols such as cholesterol or stigmasterol or a hydrophobic vitamin such as tocopherol. In a non-limiting example, a hydrophobic small molecule is cholesterol.

In some embodiments, an additional lipid or lipids comprises a neutral lipids. As a non-limiting example a neutral lipid can include any of a number of lipid species which exist either in an uncharged or neutral zwitterionic form at physiological pH. Such lipids include, for example diacylphosphatidylcholine, diacylphosphatidylethanotamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. As a non-limiting example, a neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids contain saturated fatty acids with carbon chain lengths in the range of C₁₀ to C₂₀. In another group of embodiments, lipids with mono or diunsaturated fatty acids with carbon chain lengths in the range of C₁₀ to C₂₀ are used. Additionally, lipids having mixtures of saturated and unsaturated fatty acid chains can be used. As a non-limiting example, a neutral lipid is DOPE, DSPC, POPC, DPPC or any related phosphatidylcholine. The neutral lipids may also be composed of sphingomyelin, dihydrosphingomyeline, or phospholipids with other head groups, such as serine and inositol.

In some embodiments, an additional lipid or lipids comprises a phospholipid. As a non-limiting example, the phospholipid includes 1,2-dioleoyl-sn-glycero-3-phosphoethanolamine. Additional non-limiting examples of phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and beta-acyloxyacids.

In some embodiments, an additional lipid or lipids comprises a stealth lipid. In a non-limiting example, a stealth lipid comprises a hydrophilic head and lipid moiety; in various embodiments, a stealth lipid can improve in vivo potency, increase efficacy, and/or decrease toxicity. Non-limiting examples of stealth lipids are provided, for example in WO 2011/076807.

In some embodiments, an additional lipid or lipids comprises a sterol. In a non-limiting example, a sterol is a steroid alcohol, or a member of a subgroup of steroids. In a non-limiting example, a sterol can be any of those sterols conventionally used in the field of liposome, lipid vesicle or lipid particle preparation. In a non-limiting example, a sterol is cholesterol. In a non-limiting example, a sterol is a Cholesterol, Ergosterol, Hopanoid, Phytosterol, or Steroid.

In some embodiments, an additional lipid or lipids comprises a targeting lipid or other targeting component. In non-limiting examples, a targeting lipid or other targeting component targets the lipid particles using targeting moieties that are specific to a cell type or tissue. Targeting of lipid particles using a variety of targeting moieties, such as ligands, cell surface receptors, glycoproteins, vitamins e.g., riboflavin) and monoclonal antibodies, has been previously described (see, e.g., U.S. Pat. Nos. 4,957,773 and 4,603,044). The targeting moieties can comprise the entire protein or fragments thereof. Targeting mechanisms generally require that the targeting agents be positioned on the surface of the lipid particle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002). The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)). Standard methods for coupling the target agents can be used. For example, phosphatidylethanolamine, which can be activated for attachment of target agents, or derivatized lipophilic compounds, such as lipid-derivatized bleomycin, can be used.

In various embodiments, the additional lipid or lipids comprises any lipid described herein or reported in the art. In various embodiments, the additional lipid or lipids comprises: 5-heptadecylbenzene-1,3-diol (resorcinol), cholesterol hemisuccinate (CHEMS), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1,2-diarachidoyl-sn-glycero-3-phosphocholine (DBPC), 1-palmitoyl-2-stearoyl phosphatidylcholine (PSPC), I-stearoyl-2-palmitoyl phosphatidylcholine (SPPC), 1,2-distearoyl-sn-glycero-3-phosphocholine (DAPC), 1,2-dieicosenoyl-sn-glycero-3-phosphocholine (DEPC), palmitoyloleoyl phosphatidylcholine (POPC), lysophosphatidyl choline, dilinoleoylphosphatidylcholine distearoylphophatidylethanolamine (DSPE), dimyristoyi phosphatidylethanolamine (DMPE), dipalmitoyl phosphatidylethanolamine (DPPE), palmitoyloleoyl phosphatidylethanolamine (POPE), lysophosphatidylethanolamine or a combination thereof. In one embodiment, the neutral phospholipid is selected from the group consisting of distearoylphosphatidylcholine (DSPC) and/or dimyristoyi phosphatidyl ethanolamine (DMPE).

Included in the instant disclosure is a free form of any lipid disclosed herein, as well as a pharmaceutically acceptable salt and stereoisomer thereof. Some of the isolated specific cationic lipids exemplified herein are the protonated salts of amine cationic lipids. The encompassed pharmaceutically acceptable salts not only include the isolated salts exemplified for the specific lipids described herein, but also all the typical pharmaceutically acceptable salts of the free form of any lipid disclosed herein.

The pharmaceutically acceptable salts of the instant lipids can be synthesized from the lipids of this invention which contain a basic or acidic moiety by conventional chemical methods. Generally, the salts of the basic cationic lipids are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the lipids of this disclosure include the conventional non-toxic salts of the lipids of this invention as formed by reacting a basic instant lipids with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic (TFA) and the like.

When the lipids of the present disclosure are acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Non-limiting examples include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methyl glucamine, morpholine, piperazine, piperidine, polyamine resins, procaine, purines, theobromine, triethylamine, trimethylamine tripropylamine, tromethamine and the like.

### Targeting Compound or Moiety

In some embodiments, the present disclosure pertains to compositions and methods related to a composition comprising: a biologically active agent; a lipid comprising a C₁₀-C₄₀ linear, saturated or partially unsaturated, aliphatic chain; and a targeting compound or moiety (targeting component). In some embodiments, the present disclosure pertains to compositions and methods related to a composition comprising: a biologically active agent; a lipid comprising a C₁₀-C₄₀ linear, saturated or partially unsaturated, aliphatic chain, optionally substituted with one or more C₁₋₄ aliphatic group; and a targeting compound or moiety.

In various embodiments, the composition for delivery of a biologically active agent comprises a biologically active agent; a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl; and a targeting compound or moiety.

In various embodiments, the targeting compound or moiety is selected from: an antibody, a sugar, an antigen, a small molecule, a peptide, and a cell penetrating peptide (CPP).

In some embodiments, a targeting compound or moiety is a structure capable of targeting a compound or composition to a particular cell or tissue or subset of cells or tissues. In some embodiments, a targeting moiety is designed to take advantage of cell- or tissue-specific expression of particular targets, receptors, proteins, or other subcellular components; In some embodiments, a targeting moiety is a ligand (e.g., a small molecule, antibody, peptide, protein, carbohydrate, aptamer, etc.) that targets a compound or a composition to a cell or tissue, and/or binds to a target, receptor, protein, or other subcellular component. In some embodiments, a targeting moiety targets a composition comprising a lipid and a biologically active agent to a muscle cell or tissue. In some embodiments, a targeting moiety comprises a compound that targets a muscle cell or tissue. In some embodiments, a targeting moiety comprises fetuin, epidermal growth factor, fibroblast growth factor, insulin, and/or dexamethasone, or a component or fragment or combination thereof. In some embodiments, a targeting moiety targets a composition comprising a lipid and a biologically active agent to a neuron or other cell or tissue in the neuromuscular system. In some embodiments, a targeting moiety comprises a rabies virus peptide (see Kumar et al. 2007 Nature 448: 39-43; and Hwang do et al. 2011 Biomaterials 32: 4968-4975). In some embodiments, a targeting moiety is a moiety capable of binding to a neurotransmitter transporter, a dopamine transporter, a serotonin transporter, or norepinephrine transporter, or alpha-synuclein, or a mRNA encoding any of these components (see U.S. Pat. No. 9,084,825). In some embodiments, a targeting moiety is a transferrin receptor ligand or alpha-transferrin antibody, thus reportedly making use of a transferrin receptor-mediated route across the vascular endothelium. Clark et al. 2015 Proc. Natl. Acad. Sci. USA 112: 12486-12491; Bien-Ly et al. 2014 J. Exp. Med. 211: 233-244; and Youn et al. 2014 Mol. Pharm. 11: 486-495. In some embodiments, a targeting moiety binds to an integrin. In some embodiments, a targeting moiety binds to alphaIIbeta3, e.g., on platelets. In some embodiments, a targeting moiety binds to a beta2 integrin, e.g., on a leukocyte. In some embodiments, a targeting moiety binds to an alphavbeta3, e.g., on a tumor cell. In some embodiments, a targeting moiety binds to a GPCR (G protein-coupled receptor) (see Hanyaloglu et al. 2008 Ann. Rev. Pharm. Tox. 48: 537-568). In some embodiments, a targeting moiety binds to a gastrin releasing peptide receptor, e.g., on a cancer cell (see Cornelio et al. 2007 Ann. Oncol. 18: 1457-1466). In some embodiments, a targeting moiety comprises a carbonic anhydrase inhibitor.

Antibody-targeted liposomes can be constructed using, for instance, liposomes that incorporate protein A (see, Renneisen, et al., J. Bio. Chem., 265:16337-16342 (1990) and Leonetti, et. al., Proc. Natl. Acad. Sci. (USA), 87:2448-2451 (1990). Other examples of antibody conjugation are disclosed in U.S. Pat. No. 6,027,726. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

In various embodiments, the targeting compound or moiety increases the targeting of the composition comprising a biologically active agent to a particular cell or tissue. For example, it is reported that particular cells or tissues in the body comprise particular receptors or other structures which allow the selective uptake or particular compounds. For example, muscle cells reportedly readily uptake sugars. Thus, as a non-limiting example, if delivery to a muscle cell or tissue is desired, the composition for delivery of a biologically active agent can comprise a biologically active agent; a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl; and a targeting compound or moiety, wherein the targeting compound or moiety targets a muscle cell or tissue. Such a targeting compound or moiety can comprise, as a non-limiting example, a sugar, e.g., a glucosamine (e.g., a trinary glucosamine or mono glucosamine) or mannose (e.g., a mono mannose). In various embodiments of the composition, the lipid and/or the targeting compound or moiety is conjugated to the biologically active agent. As a non-limiting example, if the biologically active agent is a nucleic acid, the nucleic acid can comprise a lipid and a targeting compound or moiety, e.g., a sugar. As a non-limiting example, if the biologically active agent is a nucleic acid, the nucleic acid can be conjugated to a lipid and/or targeting compound or moiety, e.g., a sugar. As a non-limiting example, if the biologically active agent is a nucleic acid, the nucleic acid can be conjugated on one end (e.g., the 5′ or 3′ end) to a lipid, and conjugated at the other end (e.g., the other of the 5′ or 3′ end) to a targeting compound or moiety, e.g., a sugar, e.g., a glucosamine (e.g., a trinary or mono glucosamine) or mannose (e.g., a mono mannose). In some embodiments, a targeting compound or moiety or component comprises a carbonic anhydrase inhibitor.

Optional 5′ and 3′ End Modifications for Biologically Active Agents which are Nucleic Acids

In some embodiments, the present disclosure pertains to a composition comprising a lipid and a biologically active agent, wherein the biologically active agent comprises or consists of a nucleic acid (e.g., an oligonucleotide). In some embodiments, a nucleic acid can further comprise a 5′ end or 3′ end cap (also referenced as a “modification”), which is non-nucleotidic. By describing a 5′ end cap or 3′ end cap as “non-nucleotidic”, it is meant that a nucleotide comprises three components: a phosphate, a pentose (e.g., a ribose or deoxyribose) and a nucleobase, and a 3′ end cap does not comprise all three of the components.

The 5′ end cap can be selected, as non-limiting examples, from any of: a composition comprising GalNAc; a nucleotide lacking a 5′ phosphate or 5′-OH; a nucleotide lacking a 5′ phosphate or a 5′-OH and also comprising a 2-OMe or 2′-MOE modification; 5′-deoxy-2′-O-methyl modification; 5′-OME-dT; ddT; and 5′-OTr-dT. Any 5′ end cap known in the art can be used on a CpG oligonucleotide.

The 3′ end cap can be selected, as non-limiting examples, from any of: C3, C6, C8, C10, C12, lithocholic acid, biphenyl, triethylene glycol, cyclohexyl, phenyl, adamantane, C3 amino, C7 amino, X027, X038, X050 to 52, X058 to 69, X097 to 98, X109 to 113, X1009 to 1028, and X1047 to 1049. See, for example, U.S. Pat. Nos. 8,084,600; 8,404,832; 8,404,831; 8,957,041; and WO 2015051366.

Any 3′ end cap known in the art can be used on a CpG oligonucleotide.

Any 5′ end cap can be used in combination with any 3′ end cap.

In various embodiments, the present disclosure pertains to a composition comprising a lipid and a biologically active agent, wherein the biologically active agent is a nucleic acid, and the nucleic acid comprises a 5′ end cap; a 3′ end cap; a 5′ end cap and a 3′ end cap; or neither a 5′ nor a 3′ end cap.

### Methods of Making a Composition Comprising a Lipid and a Biologically Active Agent

In some embodiments, the present disclosure pertains to compositions and methods related to a composition comprising a biologically active agent and a lipid comprising a C₁₀-C₄₀ linear, saturated or partially unsaturated, aliphatic chain, wherein the lipid is conjugated to the biologically active agent. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group, wherein the lipid is conjugated to the biologically active agent.

In some embodiments, the present disclosure pertains to compositions and methods related to a composition comprising a biologically active agent and a lipid comprising a C₁₀-C₄₀ linear, saturated or partially unsaturated, aliphatic chain, wherein the lipid is not conjugated to the biologically active agent. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group, wherein the lipid is not conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is not conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is directly conjugated to the biologically active agent (without a linker interposed between the lipid and the biologically active agent).

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is indirectly conjugated to the biologically active agent (with a linker interposed between the lipid and the biologically active agent).

Various methods of making compositions comprising lipids are known in the art.

Various methods of conjugating a lipid to various molecules are known in the art.

A non-limiting example of a method for conjugating a lipid to an oligonucleotide is provided in Example 2. Similar methods can be used to conjugate stereorandom and stereopure oligonucleotides to various lipids.

Any appropriate method known in the art can be used to produce a composition comprising a lipid and a biologically active agent as described herein.

### Linkers

In some embodiments, the present disclosure pertains to compositions and methods related to a composition comprising a biologically active agent and a lipid comprising a C₁₀-C₄₀ linear, saturated or partially unsaturated, aliphatic chain, wherein the lipid is conjugated to the biologically active agent. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group, wherein the lipid is conjugated to the biologically active agent.

In some embodiments, the present disclosure pertains to compositions and methods related to a composition comprising a biologically active agent and a lipid comprising a C₁₀-C₄₀ linear, saturated or partially unsaturated, aliphatic chain, wherein the lipid is not conjugated to the biologically active agent. In some embodiments, the present disclosure pertains to compositions and methods related to a composition comprising a biologically active agent and a lipid comprising a C₁₀-C₄₀ linear, saturated or partially unsaturated, aliphatic chain, optionally substituted with one or more C₁₋₄ aliphatic group, wherein the lipid is not conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is not conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is conjugated to the biologically active agent.

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is directly conjugated to the biologically active agent (without a linker interposed between the lipid and the biologically active agent).

In some embodiments, a composition comprises a biologically active agent and a lipid selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the lipid is indirectly conjugated to the biologically active agent (with a linker interposed between the lipid and the biologically active agent).

A linker is a moiety that connects two parts of a composition; as a non-limiting example, a linker physically connects a biologically active agent to a lipid. In some embodiments, a linker is -L^{LD}-.

Non-limiting examples of suitable linkers include: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group.

In some embodiments, a linker comprises an uncharged linker or a charged linker.

In some embodiments, a linker comprises an alkyl.

In some embodiments, a linker comprises a phosphate. In various embodiments, a phosphate can also be modified by replacement of a bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. In some embodiments, the bridging oxygen is the 3′-oxygen of a nucleoside, replacement with carbon is done. In some embodiments, the bridging oxygen is the 5′-oxygen of a nucleoside, replacement with nitrogen is done. In various embodiments, the linker comprising a phosphate comprises any one or more of: a phosphorodithioate, phosphoramidate, boranophosphonoate, or a compound of formula (I):

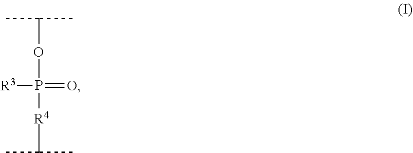

where R³ is selected from OH, SH, NH₂, BH₃, CH₃, C₁₋₆ alkyl, C₆₋₁₀ aryl, C₁₋₆ alkoxy and C₆₋₁₀ aryl-oxy, wherein C₁₋₆ alkyl and C₆₋₁₀ aryl are unsubstituted or optionally independently substituted with 1 to 3 groups independently selected from halo, hydroxyl and NH₂; and R⁴ is selected from O, S, NH, or CH₂.

In some embodiments, a linker comprises a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R₁)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, a linker is a branched linker. In some embodiments, a branchpoint of the branched linker may be at least trivalent, but may be a tetravalent, pentavalent or hexavalent atom, or a group presenting such multiple valencies. In some embodiments, a branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In one embodiment, a linker comprises at least one cleavable linking group.

As a non-limiting example, a cleavable linking group can be sufficiently stable outside the cell, but which upon entry into a target cell is cleaved to release the two parts the linker is holding together. As a non-limiting example, a cleavable linking group is cleaved at least 10 times or more, at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum). Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

As a non-limiting example, a cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a desired pH, thereby releasing the cationic lipid from the ligand inside the cell, or into the desired compartment of the cell.

As a non-limiting example, a linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

As a non-limiting example, a linker can contain a peptide bond, which can be used when targeting cell types rich in peptidases, such as liver cells and synoviocytes.

As a non-limiting example, suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. As a non-limiting example, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

In some embodiments, a linker comprises a redox cleavable linking group. As a non-limiting example, one class of cleavable linking groups are redox cleavable linking groups that are cleaved upon reduction or oxidation. A non-limiting example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. As a non-limiting example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. As a non-limiting example, candidate compounds are cleaved by at most 10% in the blood. As a non-limiting example, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In some embodiments, a linker comprises a phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Additional non-limiting examples are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. An additional non-limiting examples is —O—P(O)(OH)—O—. In various embodiments, Rk is any of: OH, SH, NH₂, BH₃, CH₃, C₁₋₆ alkyl, C₆₋₁₀ aryl, C₁₋₆ alkoxy and C₆₋₁₀ aryl-oxy, wherein C₁₋₆ alkyl and C₆₋₁₀ aryl are unsubstituted or optionally independently substituted with 1 to 3 groups independently selected from halo, hydroxyl and NH₂; and R⁴ is selected from O, S, NH, or CH₂.

In some embodiments, a linker comprises an acid cleavable linking groups are linking groups that are cleaved under acidic conditions. As a non-limiting example, acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). In an additional non-limiting example, when the carbon attached to the oxygen of the ester (the alkoxy group) is an aryl group, substituted alkyl group, or tertiary alkyl group such as dimethyl pentyl or t-butyl.

In some embodiments, a linker comprises an ester-based linking groups. As a non-limiting example, ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

In some embodiments, a linker comprises a peptide-based cleaving group. Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. As a non-limiting example, peptide-based cleavable groups do not include the amide group (—C(O)NH—). The amide group can be formed between any alkylene, alkenylene or alkynylene. A peptide bond is a special type of amide bond formed between amino acids to yield peptides and proteins. As a non-limiting example, a peptide based cleavage group can be limited to the peptide bond (i.e., the amide bond) formed between amino acids yielding peptides and proteins and does not include the entire amide functional group. As a non-limiting example, a peptide-based cleavable linking groups can have the general formula —NHCHR^{A}C(O)NHCHR^{B}C(O)—, where R^{A} and R^{B} are the R groups of the two adjacent amino acids. These candidates can be evaluated using methods analogous to those described above.

Any linker reported in the art can be used, including, as non-limiting examples, those described in: U.S. Pat. App. No. 20150265708.

In some embodiments, a lipid is conjugated to a biologically active agent using any method known in the art in accordance with the present disclosure.

Non-limiting examples of procedures for conjugating a lipid to a biologically active agent are provided in the Examples. For example, a lipid (e.g., stearic acid or turbinaric acid) can be conjugated to an oligonucleotide (e.g., WV-3473) using a C6 PO linker to produced WV-3545, 5′-Mod015L001fU*fC*fA*fA*fG*fG*mAfA*mGmA*fU*mGmGfC*fA*fU*fU*fU*fC*fU-3′ (SEQ ID NO: 2419), wherein Mod015L001 is based on stearic acid and C6 PO linker; and WV-3546, 5′-Mod020L001fU*fC*fA*fA*fG*fG*mAfA*mGmA*fU*mGmGfC*fA*fU*fU*fU*fC*fU-3′ (SEQ ID NO: 2420), wherein Mod020L001 is based on turbinaric acid and C6 PO linker; WV-3856, 5′-Mod015L001fU*fC*fA*fA*fG*fG*mAfA*mGfA*mUfG*mGfC*fA*fU*fU*fU*fC*fU-3′ (SEQ ID NO: 2421), wherein Mod015L001 is based on stearic acid and C6 PO linker; and WV-3559, 5′-Mod020L001fU*fC*fA*fA*fG*fG*mAfA*mGfA*mUfG*mGfC*fA*fU*fU*fU*fC*fU-3′ (SEQ ID NO: 2422), wherein Mod020L001 is based on turbinaric acid and C6 PO linker. These oligonucleotides were efficacious in various in vitro assays.

### Pharmaceutical Preparations

A pharmaceutical composition can comprise a lipid and a biologically active agent.

In various embodiments, the present disclosure pertains to a composition or pharmaceutical composition comprising a lipid and a biologically active agent and a pharmaceutically acceptable carrier.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. A pharmaceutical composition comprising a lipid and a biologically active agent can be delivered by intravenous infusion, subcutaneous injection, intramuscular injection, intranasal, intrathecal, topical, mucosal delivery, vaginal delivery, oral delivery, intrarectal delivery, conjunctival delivery, intraocular delivery, transcutaneous delivery, or any other modality known in the art.

This pharmaceutical composition can further comprise any component appropriate for delivery of a composition comprising a lipid and a biologically active agent. Such components include, as non-limiting examples, a pharmaceutically acceptable salt, a pharmaceutically accept carrier.

Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Pharmaceutically acceptable carriers are well known in the art. For example, acceptable carriers (e.g., pharmaceutically acceptable carriers), diluents, stabilizers, buffers, preservatives, etc., may be included as described infra.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable carriers or excipients that can be used in the manufacture of a pharmaceutical composition include, but are not limited to: binding agents, buffering agents, disintegrating agents, dispersing and/or granulating agents, inert diluents, lubricating agents, oils, preservatives, surface active agents and/or emulsifiers. Such excipients may optionally be included in pharmaceutical compositions.

Non-limiting example diluents include, but are not limited to: calcium carbonate, calcium hydrogen phosphate, calcium phosphate, calcium sulfate, cellulose, cornstarch, dicalcium phosphate, dry starch, inositol, kaolin, mannitol, microcrystalline cellulose, powdered sugar, sodium carbonate, sodium chloride, sodium phosphate lactose, sorbitol, sucrose, and/or combinations thereof.

Non-limiting example granulating and/or dispersing agents include, but are not limited to: agar, alginic acid, bentonite, calcium carbonate, calcium carboxymethyl cellulose, carboxymethyl cellulose, cation-exchange resins, cellulose and wood products, citrus pulp, clays, corn starch, cross-linked poly(vinyl-pyrrolidone) (crospovidone), cross-linked sodium carboxymethyl cellulose (e.g., croscarmellose), guar gum, magnesium aluminum silicate (e.g., VEEGUM®), methylcellulose, microcrystalline starch, natural sponge, potato starch, pregelatinized starch (e.g., starch 1500), quaternary ammonium compounds, silicates, sodium carbonate, sodium carboxymethyl starch (sodium starch glycolate), sodium lauryl sulfate, sodium starch glycolate, tapioca starch, water insoluble starch, and/or combinations thereof.

Non-limiting example surface active agents and/or emulsifiers include, but are not limited to: polyoxyethylene lauryl ether [BRIJ® 30], acacia, acrylic acid polymer, agar, alginic acid, and carboxyvinyl polymer, and propylene glycol monostearate, and SOLUTOL®), benzalkonium chloride, carbomers, carboxy polymethylene, carboxymethylcellulose sodium, carrageenan, casein, cellulosic derivatives, cetrimonium bromide, cetyl alcohol, cetylpyridinium chloride, cholesterol, cholesterol, chondrux, colloidal clays (e.g. bentonite[aluminum silicate] and VEEGUM® [magnesium aluminum silicate]), diethylene glycol monolaurate, docusate sodium, egg yolk, ethyl laurate, ethyl oleate, ethylene glycol distearate, gelatin, glyceryl monooleate, glyceryl monostearate, high molecular weight alcohols, stearyl alcohol, hydroxymethyl cellulose, hydroxypropyl cellulose, hydroxypropyl methylcellulose, lecithin, long chain amino acid derivatives, methylcellulose, natural emulsifiers, oleic acid, oleyl alcohol, pectin, PLUORINC®F 68, POLOXAMER® 188, poly(vinyl-pyrrolidone), polyacrylic acid, polyethoxylated castor oil, polyethylene glycol fatty acid esters (e.g., CREMOPHOR®), polyoxyethylene esters (e.g. polyoxyethylene monostearate [MYRJ®45], polyoxyethylene ethers, polyoxyethylene hydrogenated castor oil, polyoxyethylene sorbitan [TWEENn®60], polyoxyethylene sorbitan monooleate [TWEEN®80], polyoxymethylene stearate, polyvinyl alcohol), potassium oleate, powdered cellulose, sodium alginate, sodium lauryl sulfate, sodium oleate, sorbitan fatty acid esters (e.g. polyoxyethylene sorbitan monolaurate [TWEEN®20], sorbitan monooleate [SPAN®80]), sorbitan monopalmitate [SPAN®40], sorbitan monostearate [SPAN®60], sorbitan tristearate [SPAN®65], sucrose fatty acid esters, tragacanth, triacetin monostearate, triethanolamine oleate, wax, wool fat, xanthan, and/or combinations thereof.

Non-limiting example binding agents include, but are not limited to: starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®); and larch arabogalactan; alginates; polyethylene oxide; polyethylene glycol; inorganic calcium salts; silicic acid; polymethacrylates; waxes; water; alcohol; and combinations thereof.

Non-limiting example preservatives may include, but are not limited to: acidic preservatives, alcohol preservatives, antifungal preservatives, antimicrobial preservatives, antioxidants, chelating agents, and/or other preservatives.

Non-limiting example antioxidants include, but are not limited to: alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Non-limiting example chelating agents include: citric acid monohydrate, dipotassium edetate, disodium edetate, edetic acid, ethylenediaminetetraacetic acid (EDTA), fumaric acid, malic acid, phosphoric acid, sodium edetate, tartaric acid, and trisodium edetate,

Non-limiting example antimicrobial preservatives include, but are not limited to: benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Non-limiting example antifungal preservatives include, but are not limited to: benzoic acid, butyl paraben, ethyl paraben, hydroxybenzoic acid, methyl paraben, potassium benzoate, potassium sorbate, propyl paraben, sodium benzoate, sodium propionate, and sorbic acid.

Non-limiting example alcohol preservatives include, but are not limited to, bisphenol, chlorobutanol, ethanol, hydroxybenzoate, phenol, phenolic compounds, phenylethyl alcohol, and polyethylene glycol.

Non-limiting example acidic preservatives include, but are not limited to, acetic acid, ascorbic acid, beta-carotene, citric acid, dehydroacetic acid, phytic acid, sorbic acid, vitamin A, vitamin C, vitamin E, Other preservatives include, but are not limited to, butylated hydroxyanisol (BHA), butylated hydroxytoluened (BHT), cetrimide, deteroxime mesylate, ethylenediamine, EUXYL®, GERMABEN®II, GERMALL® 115, GLYDANT PLUS®, KATHON™, methylparaben, NEOLONE™, PHENONIP®, potassium metabisulfite, potassium sulfite, sodium bisulfite, sodium lauryl ether sulfate (SLES), sodium lauryl sulfate (SLS), sodium metabisulfite, tocopherol acetate, and tocopherol.

Non-limiting example buffering agents include, but are not limited to: acetate buffer solutions, alginic acid, aluminum hydroxide, ammonium chloride, calcium carbonate, calcium chloride, calcium citrate, calcium glubionate, calcium gluceptate, calcium gluconate, calcium glycerophosphate, calcium hydroxide phosphate, calcium lactate, calcium levulinate, citrate buffer solutions, D-gluconic acid, dibasic calcium phosphate, dibasic potassium phosphate, dibasic sodium phosphate, ethyl alcohol, isotonic saline, magnesium hydroxide, monobasic potassium phosphate, monobasic sodium phosphate, pentanoic acid, phosphate buffer solutions, phosphoric acid, potassium acetate, potassium chloride, potassium gluconate, potassium mixtures, potassium phosphate mixtures, propanoic acid, pyrogen-free water, Ringer's solution, sodium acetate, sodium bicarbonate, sodium chloride, sodium citrate, sodium lactate, sodium phosphate mixtures, tribasic calcium phosphate, tromethamine, and/or combinations thereof.

Non-limiting example lubricating agents include, but are not limited to: calcium stearate, glyceryl behenate, hydrogenated vegetable oils, leucine, magnesium lauryl sulfate, magnesium stearate, malt, polyethylene glycol, silica, sodium acetate, sodium benzoate, sodium chloride, sodium lauryl sulfate, stearic acid, talc, and/or combinations thereof.

Non-limiting example oils include, but are not limited to: almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils.

Non-limiting example oils include, but are not limited to: butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof. Non-limiting example excipients include, but are not limited to: cocoa butter and suppository waxes, coloring agents, coating agents, sweetening, flavoring, and/or perfuming agents.

As a non-limiting example, in an in vitro experiment, the results of which are shown in FIG. 1, a composition comprising a biologically active agent and a lipid was delivered to cells in muscle cell proliferation medium. As a non-limiting example, in an in vivo experiment, the results of which are shown in FIGS. 2 to 6, a composition comprising a biologically active agent and a lipid was delivered to mice subcutaneously in PBS.

Any composition comprising a lipid and a biologically active disclosed herein can be used in any pharmaceutical composition described herein or otherwise known in the art.

### Methods of Delivery of a Pharmaceutical Composition Comprising a Lipid and a Biologically Active Agent

A pharmaceutical composition comprising a lipid and a biologically active agent can be delivered using any method or device or other modality known in the art, including, but not limited, to any method of administration described herein.

As a non-limiting example: A pharmaceutical composition comprising a lipid and a biologically active agent can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, intraperitoneal, or intrathecal injection, or infusion techniques and the like. As another non-limiting example: A pharmaceutical composition comprising a lipid and a biologically active agent can be delivered by intravenous infusion, subcutaneous injection, intramuscular injection, intranasal, intrathecal, topical, mucosal delivery, vaginal delivery, oral delivery, intrarectal delivery, conjunctival delivery, intraocular delivery, transcutaneous delivery, or any other modality known in the art.

The pharmaceutical composition can comprise an therapeutically effective amount or dosage of a lipid and a biologically active agent.

Dosage levels of the order of from about 0.1 mg to about 140 mg per kilogram of body weight per day are useful in the treatment of the various conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

Any composition comprising a lipid and a biologically active agent can be used in any pharmaceutical composition described herein or otherwise known in the art, and can be used with any method of delivery described herein or otherwise known in the art.

### Methods of Treatment

A composition comprising a lipid and a biologically active agent, as used herein, can be used to treat a subject in need thereof.

In various embodiments, the biologically active agent is active against a specific target gene or gene product (such as a RNA, protein or other gene product). In various embodiments, the biologically active agent is active against a specific target gene or gene product (such as a RNA, protein or other gene product), wherein the gene or gene product at least partially mediates and/or is associated with a particular disease or disorder (e.g., a target gene- or target gene or gene product-related disorder or disease). As a non-limiting example, if a biologically active agent is an antibody, the antibody can bind to a particular target gene product, and the target gene product at least partially mediates or is associated with a disorder or disease associated with the target gene or gene product.

As used herein in the context of a composition comprising a lipid and a biologically active agent, the terms “treat,” “treatment,” and the like, refer to relief from or alleviation of pathological processes. In the context of the present disclosure insofar as it relates to any of the other conditions recited herein below (other than pathological processes mediated by expression of a target gene or gene product, such as a protein), the terms “treat,” “treatment,” and the like mean to relieve or alleviate at least one symptom associated with such condition, or to slow or reverse the progression or anticipated progression of such condition, such as slowing the progression of a lipid disorder, such as atherosclerosis.

By “lower” in the context of a disease marker or symptom is meant a statistically significant decrease in such level. The decrease can be, for example, at least 10%, at least 20%, at least 30%, at least 40% or more. If, for a particular disease, or for an individual suffering from a particular disease, the levels or expression of a target gene or gene product are elevated, treatment with a composition comprising a lipid and a biologically active agent of the present disclosure can preferably reduce the level or expression of a target gene to a level considered in the literature as within the range of normal for an individual without such disorder.

The level or expression of a target gene can be measured by evaluation of mRNA (e.g., via Northern blots or PCR), or protein (e.g., Western blots). The effect of a composition comprising a lipid and a biologically active agent on target gene expression can be determined by measuring target gene transcription rates (e.g., via Northern blots; or reverse transcriptase polymerase chain reaction or real-time polymerase chain reaction). Direct measurements can be made of levels of a target gene product, e.g. by Western blots of tissues in which the target gene is expressed.

In another embodiment of the disclosure, a composition comprising a lipid and a biologically active agent can be administered to non-human animals. For example, the compositions can be given to chickens, turkeys, livestock animals (such as sheep, pigs, horses, cattle, etc.), companion animals (e.g., cats and dogs) and can have efficacy in treatment of cancer and viral diseases. In each case, a biologically active agent would be selected to match the characteristics (e.g., structure, sequence, etc.) of the target gene or gene product of the genome of the animal.

By “treatment” is meant prophylaxis, therapy, cure, or any other change in a patient's condition indicating improvement or absence of degradation of physical condition. By “treatment” is meant treatment of target gene-related disease (e.g., cancer or viral disease), or any appropriate treatment of any other ailment the patient has. As used herein, the terms “treatment” and “treat” refer to both prophylactic or preventative treatment and curative or disease-modifying treatment, including treatment of patients at risk of contracting a disease or suspected of having a disease, as well as patients already ill or diagnosed as suffering from a condition. The terms “treatment” and “treat” also refer to the maintenance and/or promotion of health in an individual not suffering from a disease but who may be susceptible to developing an unhealthy condition, such as nitrogen imbalance or muscle loss. In one embodiment, “treatment” does not encompass prevention of a disease state. Thus, the present disclosure is useful for suppressing expression of the target gene or gene product and/or treating a target gene-related disease in an individual afflicted by an target gene-related disease, or an individual susceptible to a target gene-related disease. An individual “afflicted” by an target gene-related disease has demonstrated detectable symptoms characteristics of the disease, or had otherwise been shown clinically to have been exposed to or to carry target gene-related disease pathogens or markers. As non-limiting examples, an individual afflicted by an target gene-related disease can show outward symptoms; or can show no outward symptoms but can be shown with a clinical test to carry protein markers associated with an target gene-related disease, or proteins or genetic material associated with a pathogen in the blood.

An “effective amount” or a “therapeutically effective amount” is an amount that treats a disease or medical condition of an individual, or, more generally, provides a nutritional, physiological or medical benefit to an individual. As used herein, the phrases “therapeutically effective amount” and “prophylactically effective amount” refer to an amount that provides a therapeutic benefit in the treatment, prevention, or management of pathological processes mediated by target gene expression or an overt symptom of pathological processes mediated by target gene expression. The specific amount that is therapeutically effective can be readily determined by an ordinary medical practitioner, and may vary depending on factors known in the art, such as, e.g., the type of pathological processes mediated by target gene expression, the patient's history and age, the stage of pathological processes mediated by target gene expression, and administration of other agents that inhibit pathological processes mediated by a target gene.

In various embodiments of the disclosure, the patient is at least about 1, 5, 10, 20, 30, 40, 50, 55, 60, 65, 70, or 75 years of age. In various embodiments, the patient is no more than about 1, 5, 10, 20, 30, 40, 50, 55, 60, 65, 70, 75, 80, 90, or 100 years of age. In various embodiments the patient has a body weight of at least about 20, 30, 40, 50, 60, 70, 80, 90, 100, 120, 140, 160, 180, 200, 220, 240, 260, 280, 300, 320, 340, 360, 380 or 400 lbs. In various embodiments, the patient has a body weight of no more than about 20, 30, 40, 50, 60, 70, 80, 90, 100, 120, 140, 160, 180, 200, 220, 240, 260, 280, 300, 320, 340, 360, 380 or 400 lbs.

In various embodiments of the disclosure, the dosage [measuring only the active ingredient(s)] can be at least about 1, 5, 10, 25, 50, 100, 200, 250, 300, 250, 400, 450, 500, 550, 600, 650, 700, 750, 800, 850, 900, 950 or 1000 ng, 1, 5, 10, 25, 50, 100, 200, 250, 300, 250, 400, 450, 500, 550, 600, 650, 700, 750, 800, 850, 900, 950 or 1000 micrograms, 1, 5, 10, 25, 50, 100, 200, 250, 300, 250, 400, 450, 500, 550, 600, 650, 700, 750, 800, 850, 900, 950 or 1000 mg. In various embodiments, the dosage can be no more than about 10, 25, 50, 100, 200, 250, 300, 250, 400, 450, 500, 550, 600, 650, 700, 750, 800, 850, 900, 950 or 1000 mg. In various embodiments, the dosage can be administered at least more than once a day, daily, more than once a weekly, weekly, bi-weekly, monthly, and/or every 2, 3, 4, 5, 6, 7, 8, 9, 10, 11 or 12 months, or a combination thereof.

In various embodiments, the dosage is correlated to the body weight or body surface area of the individual. The actual dosage level can be varied to obtain an amount of active agent which is effective for a particular patient, composition and mode of administration, without being toxic to the patient. The selected dose will depend on a variety of pharmacokinetic factors, including the activity of the particular biologically active agent employed, the route of administration, the rate of excretion of biologically active agent, the duration of the treatment, other drugs, compounds and/or materials used in combination with a biologically active agent, the age, sex, weight, condition, general health and prior medical history of the patient, and like factors well known in the medical arts. A physician or veterinarian having ordinary skill in the art can readily determine the effective amount of a biologically active agent required. A suitable dose will be that amount which is the lowest dose effective to produce a therapeutic effect, or a dose low enough to produce a therapeutic effect without causing side effects.

### Use in Treating Muscle-Related Diseases and Disorders.

In various embodiments, a composition comprising a lipid and a biologically active agent can be used to treat a subject with a muscle-related disorder.

In various embodiments, a muscle-related disorder is sarcopenia, a muscle movement disorder, a muscle wasting-related disorder, muscle degeneration, muscle weakness, muscular dystrophy, Duchenne muscular dystrophy, heart failure, breathing disorder, skeletal muscle degeneration caused by malnutrition and disease, a muscle-related disease related to impaired insulin-dependent signaling, muscular dystrophy, amyotrophic lateral sclerosis, spinal muscle atrophy and spinal cord injury, ischemic muscle disease. In some embodiments, a muscle related disorder includes, for example, shoulder stiffness, frozen shoulder (stiff shoulder due to age), rheumatoid arthritis, myofascitis, neck muscle rigidity, neck-shoulder-arm syndrome, whiplash syndrome, sprain, tendon sheath inflammation, low back pain syndrome, skeletal muscle atrophy and the like.

In some embodiments, the present disclosure provides the following embodiments:

1. A composition comprising a lipid and a biologically active agent.
2. A composition comprising a lipid and a biologically active agent, characterized in that the composition delivers the biologically active agent into cells.
3. The composition of any one of the preceding embodiments, wherein the composition delivers the biologically active agent into the cytoplasm of the cells.
4. The composition of any one of the preceding embodiments, wherein the composition delivers the biologically active agent into the nucleus of the cells.
5. A composition comprising a lipid and a biologically active agent, wherein the composition delivers the biologically active agent into cells to a level higher than that observed for the biologically active agent absent the lipid.
6. A composition comprising a lipid and a biologically active agent, wherein the composition is characterized in that it delivers the biologically active agent into muscle cells.
7. The composition of any one of the preceding embodiments, wherein the composition delivers the biologically active agent into the cytoplasm of the muscle cells.
8. The composition of any one of the preceding embodiments, wherein the composition delivers the biologically active agent into the nucleus of the muscle cells.
9. The composition of any one of the preceding embodiments, wherein the composition is characterized in that when administered to a subject, the composition delivers the biologically active agent to a muscle cell in the subject.
10. The composition of any one of the preceding embodiments, wherein the composition delivers the biologically active agent into the cytoplasm of the muscle cells.
11. The composition of any one of the preceding embodiments, wherein the composition delivers the biologically active agent into the nucleus of the muscle cells.
12. A composition for delivery of a biologically active agent to a muscle cell or tissue, comprising a lipid and the biologically active agent.
13. A composition comprising a biologically active agent and a lipid selected from the list of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.
14. A composition comprising a biologically active agent and a lipid selected from:

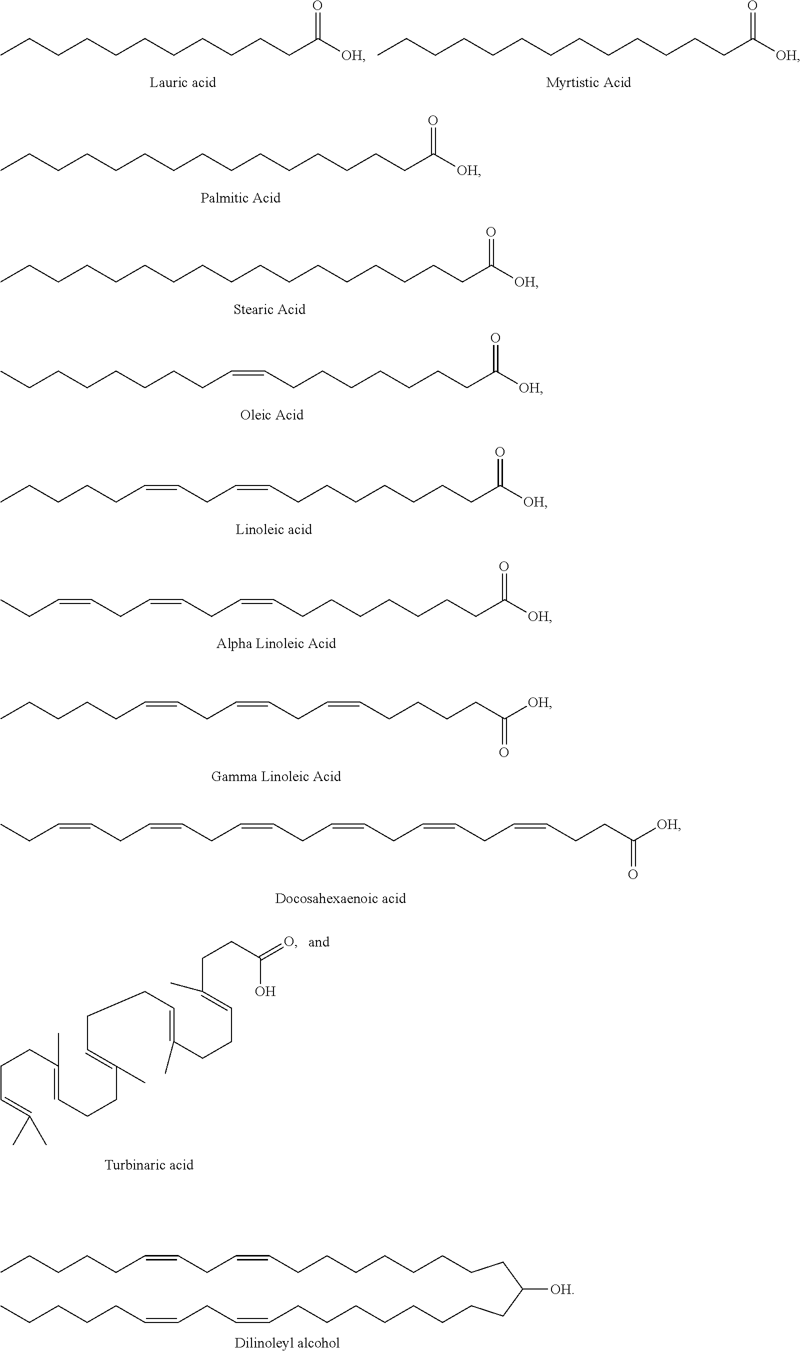

15. A composition comprising a biologically active agent and a lipid,

wherein the lipid comprises a C₁₀-C₄₀ linear, saturated or partially unsaturated, aliphatic chain, optionally substituted with one or more C₁₋₄ aliphatic group,

wherein the biologically active agent is selected from the group consisting of: a small molecule, a peptide, a protein, a component of a CRISPR-Cas system, a carbohydrate, a therapeutic agent, a chemotherapeutic agent, a vaccine, a nucleic acid, and a lipid.

16. A composition comprising a nucleic acid and a lipid, for delivery of the lipid to a muscle cell or tissue.
17. An oligonucleotide composition comprising a plurality of oligonucleotides, which share:
1) a common base sequence;
2) a common pattern of backbone linkages; and
3) a common pattern of backbone phosphorus modifications;

wherein one or more oligonucleotides of the plurality are individually conjugated to a lipid.

18. A chirally controlled oligonucleotide composition comprising a lipid and a plurality of oligonucleotides, which oligonucleotides share:
1) a common base sequence;
2) a common pattern of backbone linkages; and
3) a common pattern of backbone phosphorus modifications;

wherein:
b. the composition is chirally controlled in that the plurality of oligonucleotides share the same stereochemistry at one or more chiral internucleotidic linkages;
c. one or more oligonucleotides of the plurality are individually conjugated to a lipid; and
d. one or more oligonucleotides of the plurality are optionally and individually conjugated to a targeting compound or moiety.
   19. The composition of any one of the preceding embodiments, wherein the oligonucleotide comprises a sequence which is substantially complementary to that of a targeted element in a nucleic acid in a cell.
   20. The composition of any one of the preceding embodiments, wherein the oligonucleotide comprises a sequence which is substantially complementary to that of a targeted element in a nucleic acid in a cell in a subject.
   21. The composition of any one of the preceding embodiments, wherein the oligonucleotide comprises a sequence which is substantially complementary to that of a targeted element in a nucleic acid in a cell, wherein the nucleic acid is genomic.
   22. The composition of any one of the preceding embodiments, wherein the oligonucleotide comprises a sequence which is substantially complementary to that of a targeted element in a nucleic acid in a cell in a subject, wherein the nucleic acid is genomic.
   23. The composition of any one of the preceding embodiments, wherein the oligonucleotide comprises a sequence which is substantially complementary to that of a targeted element in a nucleic acid in a cell, wherein the targeted element is a mRNA or a portion thereof.
   24. The composition of any one of the preceding embodiments, wherein the oligonucleotide comprises a sequence which is substantially complementary to that of a targeted element in a nucleic acid in a cell in a subject, wherein the targeted element is a mRNA or a portion thereof.
   25. The composition of any one of the preceding embodiments, wherein the oligonucleotide comprises a sequence which is substantially complementary to that of a targeted element in a nucleic acid in a cell, wherein the targeted element is associated with a disease, disorder or condition.
   26. The composition of any one of the preceding embodiments, wherein the oligonucleotide comprises a sequence which is substantially complementary to that of a targeted element in a nucleic acid in a cell in a subject, wherein the targeted element is associated with a disease, disorder, or condition.
   27. The composition of any one of the preceding embodiments, wherein the oligonucleotide comprises a sequence which is substantially complementary to that of a targeted element in a nucleic acid in a muscle cell, wherein the targeted element is associated with a muscle disease, disorder, or condition.
   28. The composition of any one of the preceding embodiments, wherein the oligonucleotide comprises a sequence which is substantially complementary to that of a targeted element in a nucleic acid in a muscle cell in a subject, wherein the targeted element is associated with a muscle disease, disorder, or condition.
   29. The composition of any one of the preceding embodiments, wherein a muscle disease, disorder, or condition is DMD.
   30. The composition of any one of the preceding embodiments, wherein a targeted element in a nucleic acid is a targeted element in a transcript of dystrophin.
   31. The composition of any one of the preceding embodiments, wherein the oligonucleotides in the composition provide exon skipping of exon 51 of dystrophin.
   32. The composition of any one of embodiments 17-31, wherein the plurality of oligonucleotides share the same stereochemistry at five or more chiral internucleotidic linkages.
   33. The composition of any one of embodiments 17-31, wherein the plurality of oligonucleotides share the same stereochemistry at ten or more chiral internucleotidic linkages.
   34. The composition of any one of embodiments 17-31, wherein the plurality of oligonucleotides share the same stereochemistry at each of the chiral internucleotidic linkages so that they share a common pattern of backbone chiral centers.
   35. The composition of any one of the preceding embodiments, wherein one or more oligonucleotides of the plurality are independently conjugated to a lipid through a sugar moiety.
   36. The composition of any one of the preceding embodiments, wherein one or more oligonucleotides of the plurality are independently conjugated to a lipid through a 5′-OH on the oligonucleotide.
   37. The composition of any one of the preceding embodiments, wherein one or more oligonucleotides of the plurality are independently conjugated to a lipid through a 3′-OH on the oligonucleotide.
   38. The composition of any one of the preceding embodiments, wherein one or more oligonucleotides of the plurality are independently conjugated to a lipid through a nucleobase moiety.
   39. The composition of any one of the preceding embodiments, wherein one or more oligonucleotides of the plurality are independently conjugated to a lipid through an internucleotidic linkage.
   40. The composition of any one of the preceding embodiments, wherein each oligonucleotide of the plurality is individually conjugated to a lipid.
   41. The composition of any one of the preceding embodiments, wherein each oligonucleotide of the plurality is individually conjugated to the same lipid.
   42. The composition of any one of the preceding embodiments, wherein one or more oligonucleotides comprise two or more conjugated lipids.
   43. The composition of any one of the preceding embodiments, wherein one or more oligonucleotides comprise two or more conjugated lipids, wherein the lipids are the same.
   44. The composition of any one of embodiments 1-43, wherein one or more oligonucleotides comprises two or more conjugated lipids, wherein the lipids are different.
   45. A composition comprising a biologically active agent and a lipid, wherein the agent is any agent disclosed herein, and wherein the lipid is any lipid disclosed herein.
   46. A method of delivering an oligonucleotide to a muscle cell or tissue in a human subject, comprising:
(a) providing a composition of any one of the preceding embodiments; and
(b) administering the composition to the human subject such that the oligonucleotide is delivered to a muscle cell or tissue in the subject.
   47. A method for delivering a biologically active agent to a muscle cell or tissue comprising preparing a composition according to any one of the preceding embodiments and contacting the cell or tissue with the composition.
   48. A method of modulating the level of a transcript or gene product of a gene in a cell, the method comprising the step of contacting the cell with a composition according to any one of the preceding embodiments, wherein the biologically active agent is capable of modulating the level of the transcript or gene product.
   49. A method for inhibiting expression of a gene in a muscle cell or tissue comprising preparing a composition according to any one of the preceding embodiments and treating the muscle cell or tissue with the composition.
   50. A method for inhibiting expression of a gene in a muscle cell or tissue in a mammal comprising preparing a composition according to any one of the preceding embodiments and administering the composition to the mammal.
   51. A method of treating a disease that is caused by the over-expression of one or several proteins in a muscle cell or tissue in a subject, said method comprising the administration of a composition according to any one of the preceding embodiments to the subject.
   52. A method of treating a disease that is caused by a reduced, suppressed or missing expression of one or several proteins in a subject, said method comprising the administration of a composition according to any one of the preceding embodiments to the subject.
   53. A method for generating an immune response in a subject, said method comprising the administration of a composition according to any one of the preceding embodiments to the subject, wherein the biologically active compound is an immunomodulating nucleic acid.
   54. A method for treating a sign and/or symptom of a disease, disorder, or condition in a subject selected from cancer, a proliferative disease, disorder, or condition, a metabolic disease, disorder, or condition, an inflammatory disease, disorder, or condition, and a viral infection by providing a composition of any one of the preceding embodiments and administering the composition to the subject.
   55. A method of modulating the amount of exon skipping in a cell, the method comprising contacting the cell with a composition according to any one of the preceding embodiments, wherein the biologically active agent is capable of modulating the amount of exon skipping.
   56. A method of administering a biologically active agent to a subject in need thereof, comprising steps of providing a composition comprising the agent a lipid, and administering the composition to the subject, wherein the agent is any agent disclosed herein, and wherein the lipid is any lipid disclosed herein.
   57. A method of treating a disease in a subject, the method comprising steps of providing a composition comprising a biologically active agent and a lipid, and administering a therapeutically effective amount of the composition to the subject, wherein the agent is any agent disclosed herein, and wherein the lipid is any lipid disclosed herein, and wherein the disease is any disease disclosed herein.
   58. The composition or method of any one of the preceding embodiments, wherein a lipid comprises an R^{LD} group, wherein R^{LD} is an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—, wherein:
each R′ is independently —R, —C(O)R, —CO₂R, or —SO₂R, or:
two R′ are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, and heterocyclylene; and
each R is independently hydrogen, or an optionally substituted group selected from C₁-C₆ aliphatic, phenyl, carbocyclyl, aryl, heteroaryl, or heterocyclyl.
   59. The composition or method of any one of the preceding embodiments, wherein a lipid comprises an optionally substituted C₁₀-C₄₀ saturated or partially unsaturated aliphatic chain.
   60. The composition or method of any one of the preceding embodiments, wherein a lipid comprises an optionally substituted C₁₀-C₄₀ linear, saturated or partially unsaturated, aliphatic chain.
   61. The composition or method of any one of the preceding embodiments, wherein a lipid comprises a C₁₀-C₄₀ linear, saturated or partially unsaturated, aliphatic chain, optionally substituted with one or more C₁₋₄ aliphatic group.
   62. The composition or method of any one of the preceding embodiments, wherein a lipid comprises an unsubstituted C₁₀-C₄₀ linear, saturated or partially unsaturated, aliphatic chain.
   63. The composition or method of any one of the preceding embodiments, wherein a lipid comprises no more than one optionally substituted C₁₀-C₄₀ linear, saturated or partially unsaturated, aliphatic chain.
   64. The composition or method of any one of the preceding embodiments, wherein a lipid comprises two or more optionally substituted C₁₀-C₄₀ linear, saturated or partially unsaturated, aliphatic chain.
   65. The composition or method of any one of the preceding embodiments, wherein a lipid comprises no tricyclic or polycyclic moiety.
   66. The composition or method of any one of the preceding embodiments, wherein a lipid has the structure of R¹—COOH, wherein R¹ is an optionally substituted C₁₀-C₄₀ saturated or partially unsaturated aliphatic chain.
   67. The composition or method of any one of embodiment 16, wherein the lipid is conjugated through its carboxyl group.
   68. The composition or method according to any one of the preceding embodiments, wherein the lipid is selected from:

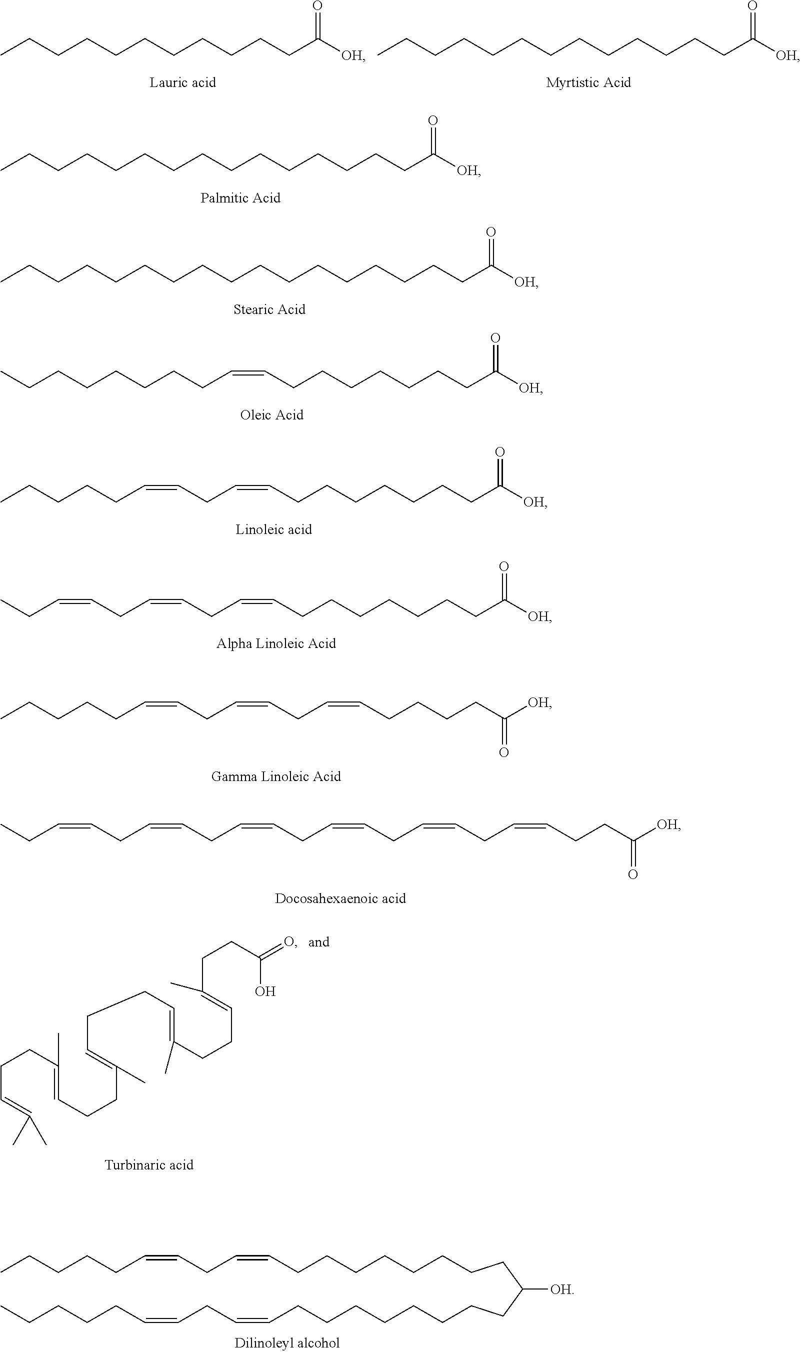

69. The composition or method of any one of the preceding embodiments, wherein the lipid is conjugated to the biologically active agent.
70. The composition or method of any one of the preceding embodiments, wherein the lipid is directly conjugated to the biologically active agent.
71. The composition or method of any one of the preceding embodiments, wherein the lipid is conjugated to the biologically active agent via a linker.
72. The composition or method of any one of the preceding embodiments, wherein the linker is -L-, wherein L is L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
each R′ is independently —R, —C(O)R, —CO₂R, or —SO₂R, or:
two R′ are taken together with their intervening atoms to form an optionally substituted aryl, carbocyclic, heterocyclic, or heteroaryl ring;
-Cy- is an optionally substituted bivalent ring selected from carbocyclylene, arylene, heteroarylene, and heterocyclylene; and
each R is independently hydrogen, or an optionally substituted group selected from C₁-C₆ aliphatic, phenyl, carbocyclyl, aryl, heteroaryl, or heterocyclyl.
   73. The composition or method of any one of the preceding embodiments, wherein the linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; and a linker comprising at least one peptide-based cleavage group.
   74. The composition or method of any one of the preceding embodiments, wherein the nucleic acid is an oligonucleotide, an antisense oligonucleotide, an RNAi agent, a miRNA, splice switching oligonucleotide (SSO), immunomodulatory nucleic acid, an aptamer, a ribozyme, a mRNA, a lncRNA, a ncRNA, an antigomir (e.g., an antagonist to a miRNA, lncRNA, ncRNA or other nucleic acid), a plasmid, a vector, or a portion thereof.
   75. The composition or method of any one of the preceding embodiments, wherein the RNAi agent is a siRNA, a shRNA, a miRNA, a sisiRNA, a meroduplex RNA (mdRNA), a DNA-RNA chimera, a siRNA comprising two mismatches (or more mismatches), a neutral siRNA, an aiRNA, or a siRNA comprising a terminal or internal spacer.
   76. The composition or method of any one of the preceding embodiments, wherein each oligonucleotide of the plurality is individually conjugated to the same lipid at the same location.
   77. The composition or method of any one of the preceding embodiments, wherein a lipid is conjugated to an oligonucleotide through a linker.
   78. The composition or method of any one of the preceding embodiments, wherein one or more oligonucleotides of the plurality are independently conjugated to a targeting compound or moiety.
   79. The composition or method of any one of the preceding embodiments, wherein one or more oligonucleotides of the plurality are independently conjugated to a lipid and a targeting compound or moiety.
   80. The composition or method of any one of the preceding embodiments, wherein one or more oligonucleotides of the plurality are independently conjugated to a lipid at one end and a targeting compound or moiety at the other.
   81. The composition or method of any one of the preceding embodiments, wherein oligonucleotides of the plurality share the same chemical modification patterns.
   82. The composition or method of any one of the preceding embodiments, wherein oligonucleotides of the plurality share the same chemical modification patterns comprising one or more base modifications.
   83. The composition or method of any one of the preceding embodiments, wherein oligonucleotides of the plurality share the same chemical modification patterns comprising one or more sugar modifications.
   84. The composition or method of any one of the preceding embodiments, wherein the common base sequence is capable of hybridizing with a transcript in a muscle cell, which transcript contains a mutation that is linked to a muscle disease, or whose level, activity and/or distribution is linked to a muscle disease.
   85. The composition or method of any one of the preceding embodiments, wherein the common base sequence is capable of hybridizing with a transcript in a muscle cell, and the composition is characterized in that when it is contacted with the transcript in a transcript splicing system, splicing of the transcript is altered relative to that observed under reference conditions selected from the group consisting of absence of the composition, presence of a reference composition, and combinations thereof.
   86. The composition or method of any one of the preceding embodiments, wherein the common base sequence hybridizes with a transcript of dystrophin.
   87. The composition or method of any one of the preceding embodiments, wherein the common base sequence hybridizes with a transcript of dystrophin, and the composition increases the production of one or more functional or partially functional proteins encoded by dystrophin.
   88. The composition or method of any one of the preceding embodiments, wherein the oligonucleotide or oligonucleotides is or are splice switching oligonucleotide or oligonucleotides.
   89. The composition or method of any one of the preceding embodiments, wherein the plurality of oligonucleotides share a common pattern of sugar modification, which comprises 3, 4, 5, 6, 7, 8, 9 or more 2′-F.
   90. The composition or method of any one of the preceding embodiments, wherein the plurality of oligonucleotides share a common pattern of sugar modification, which comprises 3 or more 2′-F.
   91. The composition or method of any one of the preceding embodiments, wherein the plurality of oligonucleotides share a common pattern of sugar modification, which comprises 3, 4, 5, 6, 7, 8, 9 or more consecutive 2′-F.
   92. The composition or method of any one of the preceding embodiments, wherein the plurality of oligonucleotides share a common pattern of sugar modification, which comprises 3 or more consecutive 2′-F.
   93. The composition or method of any one of the preceding embodiments, wherein the plurality of oligonucleotides share a common pattern of sugar modification, which comprises 3, 4, 5, 6, 7, 8, 9 or more 2′-F within the 10 nucleotides at the 5′-end.
   94. The composition or method of any one of the preceding embodiments, wherein the plurality of oligonucleotides share a common pattern of sugar modification, which comprises 3 or more 2′-F within the 10 nucleotides at the 5′-end.
   95. The composition or method of any one of the preceding embodiments, wherein the plurality of oligonucleotides share a common pattern of sugar modification, which comprises 3, 4, 5, 6, 7, 8, 9 or more consecutive 2′-F at the 5′-end.
   96. The composition or method of any one of the preceding embodiments, wherein the plurality of oligonucleotides share a common pattern of sugar modification, which comprises 3 or more consecutive 2′-F at the 5′-end.
   97. The composition or method of any one of the preceding embodiments, wherein the plurality of oligonucleotides share a common pattern of sugar modification, which comprises 3, 4, 5, 6, 7, 8, 9 or more consecutive 2′-F within the 10 nucleotides at the 5′-end.
   98. The composition or method of any one of the preceding embodiments, wherein the plurality of oligonucleotides share a common pattern of sugar modification, which comprises 3 or more consecutive 2′-F within the 10 nucleotides at the 5′-end.
   99. The composition or method of any one of the preceding embodiments, wherein the plurality of oligonucleotides share a common pattern of sugar modification, which comprises 3, 4, 5, 6, 7, 8, 9 or more 2′-F within the 10 nucleotides at the 3′-end.
   100. The composition or method of any one of the preceding embodiments, wherein the plurality of oligonucleotides share a common pattern of sugar modification, which comprises 3 or more 2′-F within the 10 nucleotides at the 3′-end.
   101. The composition or method of any one of the preceding embodiments, wherein the plurality of oligonucleotides share a common pattern of sugar modification, which comprises 3, 4, 5, 6, 7, 8, 9 or more consecutive 2′-F at the 3′-end.
   102. The composition or method of any one of the preceding embodiments, wherein the plurality of oligonucleotides share a common pattern of sugar modification, which comprises 3 or more consecutive 2′-F at the 3′-end.
   103. The composition or method of any one of the preceding embodiments, wherein the plurality of oligonucleotides share a common pattern of sugar modification, which comprises 3, 4, 5, 6, 7, 8, 9 or more consecutive 2′-F within the 10 nucleotides at the 3′-end.
   104. The composition or method of any one of the preceding embodiments, wherein the plurality of oligonucleotides share a common pattern of sugar modification, which comprises 3 or more consecutive 2′-F within the 10 nucleotides at the 3′-end.
   105. The composition or method of any one of the preceding embodiments, wherein the plurality of oligonucleotides share a common pattern of sugar modification, which comprises 5 or more 2′-F within the 10 nucleotides at the 5′-end.
   106. The composition or method of any one of the preceding embodiments, wherein the plurality of oligonucleotides share a common pattern of sugar modification, which comprises 5 or more consecutive 2′-F within the 10 nucleotides at the 5′-end.
   107. The composition or method of any one of the preceding embodiments, wherein the plurality of oligonucleotides share a common pattern of sugar modification, which comprises 5 or more 2′-F within the 10 nucleotides at the 3′-end.
   108. The composition or method of any one of the preceding embodiments, wherein the plurality of oligonucleotides share a common pattern of sugar modification, which comprises 6 or more consecutive 2′-F within the 10 nucleotides at the 5′-end.
   109. The composition or method of any one of the preceding embodiments, wherein the plurality of oligonucleotides share a common pattern of sugar modification, which comprises 6 or more 2′-F within the 10 nucleotides at the 3′-end.
   110. The composition or method of any one of the preceding embodiments, wherein the plurality of oligonucleotides share a common pattern of sugar modification, which comprises 7 or more consecutive 2′-F at the 5′-end.
   111. The composition or method of any one of the preceding embodiments, wherein the plurality of oligonucleotides share a common pattern of sugar modification, which comprises 7 or more consecutive 2′-F at the 3′-end.
   112. The composition or method of any one of the preceding embodiments, wherein the plurality of oligonucleotides share a common pattern of sugar modification, which comprises 3 or more consecutive 2′-F at the 5′-end, 3 or more consecutive 2′-F at the 3′-end, and 3 or more 2′-OR between the 5′-end 2′-F and the 3′-end 2′-F modifications.
   113. The composition or method of any one of the preceding embodiments, wherein the plurality of oligonucleotides share a common pattern of sugar modification, which comprises 3 or more 2′-F at the 5′-end, 3 or more 2′-F at the 3′-end, and 3 or more 2′-OR between the 5′-end 2′-F and the 3′-end 2′-F modifications.
   114. The composition or method of any one of the preceding embodiments, wherein the plurality of oligonucleotides share a common pattern of sugar modification, which comprises 7 or more 2′-F within the 10 nucleotides at the 3′-end.
   115. The composition or method of any one of the preceding embodiments, wherein the plurality of oligonucleotides share a common pattern of sugar modification, which comprises 5 or more consecutive 2′-F within the 10 nucleotides at the 3′-end.
   116. The composition or method of any one of the preceding embodiments, wherein the plurality of oligonucleotides share a common pattern of sugar modification, which comprises 7 or more consecutive 2′-F at the 3′-end.
   117. The composition or method of any one of the preceding embodiments, wherein the plurality of oligonucleotides comprises a 5′-wing-core-wing-3′ structure, wherein each wing region independently comprises 3 to 10 nucleosides, and the core region independently comprises 3 to 10 nucleosides.
   118. The composition or method of any one of the preceding embodiments, wherein the plurality of oligonucleotides comprises a 5′-wing-core-3′ or a 5′-core-wing-3′ structure, wherein each wing region independently comprises 3 to 10 nucleosides, and the core region independently comprises 3 to 10 nucleosides.
   119. The composition or method of any one of the preceding embodiments, wherein a 5′-wing region comprises 3, 4, 5, 6, 7, 8, 9 or more 2′-F.
   120. The composition or method of any one of the preceding embodiments, wherein a 5′-wing region comprises 3 or more 2′-F.
   121. The composition or method of any one of the preceding embodiments, wherein a 5′-wing region comprises 5 or more 2′-F.
   122. The composition or method of any one of the preceding embodiments, wherein a 5′-wing region comprises 3, 4, 5, 6, 7, 8, 9 or more consecutive 2′-F.
   123. The composition or method of any one of the preceding embodiments, wherein a 5′-wing region comprises 3 or more consecutive 2′-F.
   124. The composition or method of any one of the preceding embodiments, wherein a 5′-wing region comprises 5 or more consecutive 2′-F.
   125. The composition or method of any one of the preceding embodiments, wherein a 5′-wing region comprises 10%, 20%, 30%, 40%, 50%, 60%, 70%, 80%, 90% or more 2′-F.
   126. The composition or method of any one of the preceding embodiments, wherein each sugar of a 5′-wing region comprises a 2′-F.
   127. The composition or method of any one of the preceding embodiments, wherein a 5′-wing region comprises 3 or more chiral internucleotidic linkages.
   128. The composition or method of any one of the preceding embodiments, wherein a 5′-wing region comprises 3 or more consecutive chiral internucleotidic linkages.
   129. The composition or method of any one of the preceding embodiments, wherein a 5′-wing region comprises 10% or more chiral internucleotidic linkages.
   130. The composition or method of any one of the preceding embodiments, wherein each internucleotidic linkage of a 5′-wing region is chiral.
   131. The composition or method of any one of the preceding embodiments, wherein each internucleotidic linkage of a 5′-wing region is a phosphorothioate linkage.
   132. The composition or method of any one of the preceding embodiments, wherein a 5′-wing region comprises 5 or more Rp chiral internucleotidic linkages.
   133. The composition or method of any one of the preceding embodiments, wherein a 5′-wing region comprises 5 or more Rp consecutive internucleotidic linkages.
   134. The composition or method of any one of the preceding embodiments, wherein a 5′-wing region comprises 10%, 20%, 30%, 40%, 50%, 60%, 70%, 80%, 90% or more Rp internucleotidic linkages.
   135. The composition or method of any one of the preceding embodiments, wherein each internucleotidic linkage of a 5′-wing region is Rp.
   136. The composition or method of any one of the embodiments 1-134, wherein a 5′-wing region comprises 5 or more Sp chiral internucleotidic linkages.
   137. The composition or method of any one of the embodiments 1-134, wherein a 5′-wing region comprises 5 or more Sp consecutive internucleotidic linkages.
   138. The composition or method of any one of the embodiments 1-134, wherein a 5′-wing region comprises 10%, 20%, 30%, 40%, 50%, 60%, 70%, 80%, 90% or more Sp internucleotidic linkages.
   139. The composition or method of any one of the embodiments 1-134, wherein each internucleotidic linkage of a 5′-wing region is Sp.
   140. The composition or method of any one of the preceding embodiments, wherein a 3′-wing region comprises 3, 4, 5, 6, 7, 8, 9 or more 2′-F.
   141. The composition or method of any one of the preceding embodiments, wherein a 3′-wing region comprises 3 or more 2′-F.
   142. The composition or method of any one of the preceding embodiments, wherein a 3′-wing region comprises 5 or more 2′-F.
   143. The composition or method of any one of the preceding embodiments, wherein a 3′-wing region comprises 3, 4, 5, 6, 7, 8, 9 or more consecutive 2′-F.
   144. The composition or method of any one of the preceding embodiments, wherein a 3′-wing region comprises 3 or more consecutive 2′-F.
   145. The composition or method of any one of the preceding embodiments, wherein a 3′-wing region comprises 5 or more consecutive 2′-F.
   146. The composition or method of any one of the preceding embodiments, wherein a 3′-wing region comprises 10%, 20%, 30%, 40%, 50%, 60%, 70%, 80%, 90%, or more 2′-F.
   147. The composition or method of any one of the preceding embodiments, wherein each sugar of a 3′-wing region comprises a 2′-F.
   148. The composition or method of any one of the preceding embodiments, wherein a 3′-wing region comprises 3 or more chiral internucleotidic linkages.
   149. The composition or method of any one of the preceding embodiments, wherein a 3′-wing region comprises 5 or more consecutive internucleotidic linkages.
   150. The composition or method of any one of the preceding embodiments, wherein a 3′-wing region comprises 10%, 20%, 30%, 40%, 50%, 60%, 70%, 80%, 90% or more chiral internucleotidic linkages.
   151. The composition or method of any one of the preceding embodiments, wherein each internucleotidic linkage of a 3′-wing region is chiral.
   152. The composition or method of any one of the preceding embodiments, wherein each internucleotidic linkage of a 3′-wing region is a phosphorothioate linkage.
   153. The composition or method of any one of the preceding embodiments, wherein a 3′-wing region comprises 3 or more Rp chiral internucleotidic linkages.
   154. The composition or method of any one of the preceding embodiments, wherein a 3′-wing region comprises 5 or more Rp consecutive internucleotidic linkages.
   155. The composition or method of any one of the preceding embodiments, wherein a 3′-wing region comprises 10%, 20%, 30%, 40%, 50%, 60%, 70%, 80%, 90% or more Rp internucleotidic linkages.
   156. The composition or method of any one of the preceding embodiments, wherein each internucleotidic linkage of a 3′-wing region is Rp.
   157. The composition or method of any one of the embodiments 1-155, wherein a 3′-wing region comprises 3 or more Sp chiral internucleotidic linkages.
   158. The composition or method of any one of the embodiments 1-155, wherein a 3′-wing region comprises 5 or more Sp consecutive internucleotidic linkages.
   159. The composition or method of any one of the embodiments 1-155, wherein a 3′-wing region comprises 10%, 20%, 30%, 40%, 50%, 60%, 70%, 80%, 90% or more Sp internucleotidic linkages.
   160. The composition or method of any one of the embodiments 1-155, wherein each internucleotidic linkage of a 3′-wing region is Sp.
   161. The composition or method of any one of the preceding embodiments, wherein the 5′-wing and the 3′-wing have the same length, pattern of chemical modifications, pattern of backbone internucleotidic linkages, and pattern of backbone chiral centers.
   162. The composition or method of any one of the preceding embodiments, wherein the internucleotidic linkage between the 5′-wing region and the core region is a chiral internucleotidic linkage.
   163. The composition or method of any one of the preceding embodiments, wherein the internucleotidic linkage between the 5′-wing region and the core region is a phosphorothioate linkage.
   164. The composition or method of any one of the preceding embodiments, wherein the internucleotidic linkage between the 5′-wing region and the core region is an Rp phosphorothioate linkage.
   165. The composition or method of any one of the preceding embodiments, wherein the internucleotidic linkage between the 3′-wing region and the core region is a chiral internucleotidic linkage.
   166. The composition or method of any one of the preceding embodiments, wherein the internucleotidic linkage between the 3′-wing region and the core region is a phosphorothioate linkage.
   167. The composition or method of any one of the preceding embodiments, wherein the internucleotidic linkage between the 3′-wing region and the core region is an Rp phosphorothioate linkage.
   168. The composition or method of any one of the preceding embodiments, wherein a core region comprises 3, 4, 5, 6, 7, 8, 9 or more 2′-OR.
   169. The composition or method of any one of the preceding embodiments, wherein a core region comprises 3 or more 2′-OR.
   170. The composition or method of any one of the preceding embodiments, wherein a core region comprises 5 or more consecutive 2′-OR.
   171. The composition or method of any one of the preceding embodiments, wherein a core region comprises 10%, 20%, 30%, 40%, 50%, 60%, 70%, 80%, 90% or more 2′-OR.
   172. The composition or method of any one of the preceding embodiments, wherein each sugar of a core region comprises a 2′-OR.
   173. The composition or method of any one of the preceding embodiments, wherein a 2′-OR modification is a 2′-OMe modification.
   174. The composition or method of any one of the preceding embodiments, wherein a core region comprises 3 or more chiral internucleotidic linkages.
   175. The composition or method of any one of the preceding embodiments, wherein a core region comprises 5 or more consecutive chiral internucleotidic linkages.
   176. The composition or method of any one of the preceding embodiments, wherein a core region comprises 10% or more chiral internucleotidic linkages.
   177. The composition or method of any one of the preceding embodiments, wherein each internucleotidic linkage of a core region is chiral.
   178. The composition or method of any one of the preceding embodiments, wherein each internucleotidic linkage of a core region is a phosphorothioate linkage.
   179. The composition or method of any one of the preceding embodiments, wherein a core region comprises 3, 4, 5, 6, 7, 8, 9 or more Sp chiral internucleotidic linkages.
   180. The composition or method of any one of the preceding embodiments, wherein a core region comprises 3 or more Sp chiral internucleotidic linkages.
   181. The composition or method of any one of the preceding embodiments, wherein a core region comprises 5 or more Sp consecutive internucleotidic linkages.
   182. The composition or method of any one of the preceding embodiments, wherein a core region comprises 10%, 20%, 30%, 40%, 50%, 60%, 70%, 80%, 90% or more Sp internucleotidic linkages.
   183. The composition or method of any one of the preceding embodiments, wherein each internucleotidic linkage of a core region is Sp.
   184. The composition or method of any one of embodiments 1-182, wherein a core region comprises 3, 4, 5, 6, 7, 8, 9 or more Rp chiral internucleotidic linkages.
   185. The composition or method of any one of embodiments 1-182, wherein a core region comprises 3 or more Rp chiral internucleotidic linkages.
   186. The composition or method of any one of embodiments 1-182, wherein a core region comprises 5 or more Rp consecutive internucleotidic linkages.
   187. The composition or method of any one of embodiments 1-182, wherein a core region comprises 10%, 20%, 30%, 40%, 50%, 60%, 70%, 80%, 90% or more Rp internucleotidic linkages.
   188. The composition or method of any one of embodiments 1-178, wherein each internucleotidic linkage of a core region is Rp.
   189. The composition or method of any one of the preceding embodiments, wherein a 5′-wing region comprises 10% or more Sp internucleotidic linkages.
   190. The composition or method of any one of the preceding embodiments, wherein a 3′-wing region comprises 10% or more Sp internucleotidic linkages.
   191. The composition or method of any one of the preceding embodiments, wherein the internucleotidic linkage between the 5′-wing region and the core region is an Sp phosphorothioate linkage.
   192. The composition or method of any one of the preceding embodiments, wherein the internucleotidic linkage between the 3′-wing region and the core region is an Sp phosphorothioate linkage.
   193. The composition or method of any one of the preceding embodiments, wherein the nucleic acid is a splice switching oligonucleotide (SSO).
   194. The composition or method of any one of the preceding embodiments, wherein the nucleic acid is a splice switching oligonucleotide (SSO) which targets dystrophin.
   195. The composition or method of any one of the preceding embodiments, wherein the nucleic acid is a splice switching oligonucleotide (SSO) which targets dystrophin exon 51, 45, 53 or 44.
   196. The composition or method of any one of the preceding embodiments, wherein the nucleic acid is a splice switching oligonucleotide (SSO) which targets dystrophin exon 51.
   197. The composition or method of any one of the preceding embodiments, wherein the immunomodulatory nucleic acid is a CpG oligonucleotide.
   198. The composition or method of any one of the preceding embodiments, wherein the immunomodulatory nucleic acid is a CpG oligonucleotide which is capable of agonizing an immune response which is TLR9-mediated or TLR9-associated.
   199. The composition or method of any one of the preceding embodiments, wherein the immunomodulatory nucleic acid is a CpG oligonucleotide which is capable of antagonizing an immune response which is TLR9-mediated or TLR9-associated.
   200. The composition or method of any one of the preceding embodiments, wherein the oligonucleotide comprises a strand of about 14 to about 49 nucleotides.
   201. The composition or method of any one of the preceding embodiments, where the oligonucleotide further comprises a second strand.
   202. The composition or method of any one of the preceding embodiments, wherein the oligonucleotide comprises at least one modification to a base, sugar or internucleoside linkage.
   203. The composition or method of any one of the preceding embodiments, wherein the modification is a sugar modifications at the 2′ carbon.
   204. The composition or method of any one of the preceding embodiments, wherein the modification is a sugar modifications at the 2′ carbon selected from: 2′-MOE, 2′-OMe, and 2′-F.
   205. The composition or method of any one of the preceding embodiments, wherein the biologically active agent is a nucleic acid.
   206. The composition or method of any one of the preceding embodiments, wherein the biologically active agent is an immunomodulatory nucleic acid.
   207. The composition or method of any one of the preceding embodiments, wherein the biologically active agent is a CpG oligonucleotide that agonizes or antagonizes an immune response
   208. The composition or method of any one of the preceding embodiments, wherein the biologically active agent is an CpG oligonucleotide that agonizes or antagonizes an immune response which is TLR9-mediated or TLR9-associated.
   209. The composition or method of any one of the preceding embodiments, wherein the biologically active agent is a small molecule, and wherein the small molecule is hydrophobic
   210. The composition or method of any one of the preceding embodiments, wherein the biologically active agent is a hydrophobic small molecule selected from the group consisting of a sterol and a hydrophobic vitamin.
   211. The composition or method of any one of the preceding embodiments, wherein the biologically active agent is cholesterol.
   212. The composition or method of any one of the preceding embodiments, wherein the biologically active agent is a protein selected from the group consisting of a nucleoprotein, a mucoprotein, a lipoprotein, a synthetic polypeptide, a small molecule linked to a protein and a glycoprotein.
   213. The composition or method of any one of the preceding embodiments, wherein the biologically active agent is a nucleic acid in the form of a single stranded or partially double stranded oligomer or a polymer composed of ribonucleotides.
   214. The composition or method of any one of the preceding embodiments, wherein the biologically active agent is a nucleic acid selected from the group consisting of miRNA, antisense oligonucleotides, siRNA, immune-stimulatory oligonucleotides, aptamers, Piwi-interacting RNAs (piRNAs), small nucleolar RNAs (snoRNAs), ribozymes, and plasmids encoding a specific gene or siRNA.
   215. The composition or method of any one of the preceding embodiments, wherein the cell or tissue is a muscle cell or tissue.
   216. The composition or method of any one of the preceding embodiments, wherein the biologically active agent is an oligonucleotide.
   217. The composition or method of any one of the preceding embodiments, wherein the biologically active agent is an oligonucleotide which mediates exon skipping.
   218. The composition or method of any one of the preceding embodiments, wherein the biologically active agent is a stereodefined oligonucleotide which mediates exon skipping.
   219. The composition or method of any one of the preceding embodiments, wherein the disease or disorder is a muscle-related disease or disorder.
   220. The composition or method of any one of the preceding embodiments, wherein the muscle-related disorder is sarcopenia, a muscle movement disorder, a muscle wasting-related disorder, muscle degeneration, muscle weakness, muscular dystrophy, Duchenne muscular dystrophy, heart failure, breathing disorder, skeletal muscle degeneration caused by malnutrition and disease, a muscle-related disease related to impaired insulin-dependent signaling, amyotrophic lateral sclerosis, spinal muscle atrophy and spinal cord injury, ischemic muscle disease.
   221. The composition or method of any one of the preceding embodiments, wherein the cell or tissue is a muscle cell or tissue, wherein the biologically active agent is a stereodefined oligonucleotide which is a splice switching oligonucleotide, and wherein the subject is afflicted with a muscle disorder.
   222. The composition or method of any one of the preceding embodiments, wherein the cell or tissue is a muscle cell or tissue, wherein the biologically active agent is a stereodefined oligonucleotide which is a splice switching oligonucleotide, and wherein the subject is afflicted with muscular dystrophy.
   223. The composition or method of any one of the preceding embodiments, wherein the cell or tissue is a muscle cell or tissue, wherein the biologically active agent is a stereodefined oligonucleotide which is a splice switching oligonucleotide, and wherein the subject is afflicted with Duchenne muscular dystrophy.
   224. The composition or method of any one of the preceding embodiments, wherein sequences of the oligonucleotides comprise or consist of a sequence listed in tables in the Specification.
   225. The composition or method of any one of the preceding embodiments, wherein sequences of the oligonucleotides comprise or consist of UCAAGGAAGAUGGCAUUUCU (SEQ ID NO: 1).
   226. The composition or method of any one of the preceding embodiments, wherein the lipid comprises an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.
   227. The composition or method of any one of the preceding embodiments, wherein the lipid comprises an optionally substituted C₁₀-C₈₀ saturated or partially unsaturated, aliphatic chain.
   228. The composition or method of any one of the preceding embodiments, wherein the lipid comprises an optionally substituted C₁₀-C₈₀ linear, saturated or partially unsaturated, aliphatic chain.
   229. The composition or method of any one of the preceding embodiments, wherein the lipid comprises an optionally substituted C₁₀-C₆₀ saturated or partially unsaturated, aliphatic chain.
   230. The composition or method of any one of the preceding embodiments, wherein the lipid comprises an optionally substituted C₁₀-C₆₀ linear, saturated or partially unsaturated, aliphatic chain.
   231. The composition or method of any one of the preceding embodiments, wherein the lipid comprises an optionally substituted C₁₀-C₄₀ saturated or partially unsaturated, aliphatic chain.
   232. The composition or method of any one of the preceding embodiments, wherein the lipid comprises an optionally substituted C₁₀-C₄₀ linear, saturated or partially unsaturated, aliphatic chain.
   233. The composition or method of any one of the preceding embodiments, wherein the lipid comprises an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.
   234. The composition or method of any one of the preceding embodiments, wherein the lipid comprises an optionally substituted C₁₀-C₈₀ saturated or partially unsaturated, aliphatic chain.
   235. The composition or method of any one of the preceding embodiments, wherein the lipid comprises an optionally substituted C₁₀-C₆₀ linear, saturated or partially unsaturated, aliphatic chain.
   236. The composition or method of any one of the preceding embodiments, wherein the lipid comprises an optionally substituted C₁₀-C₄₀ linear, saturated or partially unsaturated, aliphatic chain.
   237. The composition or method of any one of the preceding embodiments, wherein the lipid comprises an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.
   238. The composition or method of any one of the preceding embodiments, wherein the lipid comprises an optionally substituted C₁₀-C₄₀ saturated or partially unsaturated, aliphatic chain.
   239. The composition or method of any one of the preceding embodiments, wherein the lipid comprises an optionally substituted C₁₀-C₄₀ linear, saturated or partially unsaturated, aliphatic chain.
   240. The composition or method of any one of the preceding embodiments, wherein the composition further comprises one or more additional components selected from: a polynucleotide, carbonic anhydrase inhibitor, a dye, an intercalating agent, an acridine, a cross-linker, psoralene, mitomycin C, a porphyrin, TPPC4, texaphyrin, Sapphyrin, a polycyclic aromatic hydrocarbon phenazine, dihydrophenazine, an artificial endonuclease, a chelating agent, EDTA, an alkylating agent, a phosphate, an amino, a mercapto, a PEG, PEG-40K, MPEG, [MPEG]₂, a polyamino, an alkyl, a substituted alkyl, a radiolabeled marker, an enzyme, a hapten biotin, a transport/absorption facilitator, aspirin, vitamin E, folic acid, a synthetic ribonuclease, a protein, a glycoprotein, a peptide, a molecule having a specific affinity for a co-ligand, an antibody, a hormone, a hormone receptor, a non-peptidic species, a lipid, a lectin, a carbohydrate, a vitamin, a cofactor, or a drug.
   241. The composition or method of any one of the preceding embodiments, wherein the lipid comprises a C₁₀-C₈₀ linear, saturated or partially unsaturated, aliphatic chain.
   242. The composition or method of any one of the preceding embodiments, wherein the composition further comprises a linker linking the biologically active agent and the lipid, wherein the linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group.
   243. The composition or method of any one of the preceding embodiments, wherein the biologically active agent comprises or consists of or is an oligonucleotide or oligonucleotide composition or chirally controlled oligonucleotide composition.
   244. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of any oligonucleotide described herein.
   245. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of any oligonucleotide listed in Table 4A.
   246. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of a splice-switching oligonucleotide.
   247. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of an exon in the dystrophin gene.
   248. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of an oligonucleotide capable of skipping or mediating skipping of exon 51 in the dystrophin gene.
   249. The composition or method of any one of the preceding embodiments, wherein the biologically active agent comprises or consists of or is an oligonucleotide or oligonucleotide composition or chirally controlled oligonucleotide composition, wherein the sequence of the oligonucleotide comprises or consists of the sequence of any of: WV-887, WV-896, WV-1709, WV-1710, WV-1714, WV-2095, WV-2100, WV-2106, WV-2107, WV-2108, WV-2109, WV-2223, WV-2224, WV-2225, WV-2226, WV-2227, WV-2228, WV-2229, WV-2230, WV-2438, WV-2444, WV-2445, WV-2526, WV-2527, WV-2528, WV-2529, WV-2530, WV-2531, WV-2533, WV-2578, WV-2580, WV-2587, WV-3047, WV-3152, WV-3472, WV-3473, WV-3507, WV-3508, WV-3509, WV-3510, WV-3511, WV-3512, WV-3513, WV-3514, WV-3515, WV-3545, or WV-3546.
   250. The composition or method of any one of the preceding embodiments, wherein the sequence of an oligonucleotide includes any one or more of: base sequence (including length); pattern of chemical modifications to sugar and base moieties; pattern of backbone linkages; pattern of natural phosphate linkages, phosphorothioate linkages, phosphorothioate triester linkages, and combinations thereof; pattern of backbone chiral centers; pattern of stereochemistry (Rp/Sp) of chiral internucleotidic linkages; pattern of backbone phosphorus modifications; pattern of modifications on the internucleotidic phosphorus atom, such as —S—, and -L-R¹ of formula I.
   251. The composition or method of any one of the preceding embodiments, wherein the muscle cell or tissue is selected from: skeletal muscle, smooth muscle, heart muscle, thoracic diaphragm, gastrocnemius, quadriceps, triceps, and/or heart.
   252. The method of any one of the preceding embodiments, wherein the method delivers the biologically active agent into the cytoplasm of a cell.
   253. The method of any one of the preceding embodiments, wherein the method delivers the biologically active agent into the nucleus of a cell.
   254. The composition or method of any one of the preceding embodiments, wherein the chiral internucleoside linkage is a phosphorothioate.
   255. The composition or method of any one of the preceding embodiments, wherein a common base sequence hybridizes with a transcript of dystrophin, myostatin, Huntingtin, a myostatin receptor, ActRIIB, ActRIIA, DMPK, Malat1, SMN2, dystrophia myotonica protein kinase (DMPK), Proprotein convertase subtilisin/kexin type 9 (PCSK9), SMAD7 or KRT14 (Keratin 14).
   256. The composition or method of any one of the preceding embodiments, wherein the composition delivers the biologically active agent into cells to a level higher than that observed for the biologically active agent absent the lipid.
   257. The composition or method of any one of the preceding embodiments, characterized in that the composition has higher hTLR9 antagonist activity than that observed for the composition absent the lipid.
   258. A method for reducing hTLR9 agonist activities, comprising conjugating a biologically active agent to one or more lipids.
   259. A method for increasing hTLR9 antagonist activities, comprising conjugating a biologically active agent to one or more lipids.
   260. The method of any one of embodiments 258-259, characterized in that the agonist activities are reduced, or the antagonist activities are increased, compared to the biological active agent absent the lipid.
   261. The method of any one of embodiments 258-260, wherein the biological active agent is an oligonucleotides.
   262. The method of any one of embodiments 258-261, wherein the biological active agent is an oligonucleotide of any one of the preceding embodiments.
   263. The method of any one of embodiments 258-262, wherein the lipid is a lipid of any one of the preceding embodiments.
   264. The composition or method of any one of the preceding embodiments, wherein a lipid is conjugated to a biologically active agent via a linker.
   265. The composition or method of any one of the preceding embodiments, wherein the linker is -L^{LD}-.
   266. The composition or method of any one of the preceding embodiments, wherein the linker is -L-.
   267. The composition or method of any one of the preceding embodiments, wherein the linker is —NH—(CH₂)₆—.
   268. The composition or method of any one of the preceding embodiments, wherein the linker is —C(O)—NH—(CH₂)₆—P(O)(O—)—.
   269. The composition or method of any one of the preceding embodiments, wherein the linker is —C(O)—NH—(CH₂)₆—P(O)(S—)—.
   270. The composition of embodiment 268 or 269, wherein the lipid is a fatty acid which is connected to the linker through formation of the amide group —C(O)—NH—, and the oligonucleotide is connected to the linker through formation of a phosphate or phosphorothioate linkage between its 5′-OH or 3′-OH with —P(O)(O—)— or —P(O)(S—)— of the linker.
   271. The composition of embodiment 268 or 269, wherein the lipid is a fatty acid which is connected to the linker through formation of the amide group —C(O)—NH—, and the oligonucleotide is connected to the linker through formation of a phosphate or phosphorothioate linkage between its 5′-OH with —P(O)(O—)— or —P(O)(S—)— of the linker.
   272. The composition of embodiment 268 or 269, wherein the lipid is a fatty acid which is connected to the linker through formation of the amide group —C(O)—NH—, and the oligonucleotide is connected to the linker through formation of a phosphate or phosphorothioate linkage between its 3′-OH with —P(O)(O—)— or —P(O)(S—)— of the linker.
   273. The composition of any one of the preceding embodiments, further comprising one or more targeting components.
   274. A composition comprising a plurality of compounds having the structure of:
A^{c}-[-L^{LD}-(R^{LD})ₐ]_{b} or [(A^{c})ₐ-L^{LD}]_{b}-R^{LD}, or a salt thereof,
   wherein:
A^{c} is a biologically active agent;
a is 1-1000;
b is 1-1000;
each L^{LD} is independently a linker moiety; and
each R^{LD} is independently a lipid moiety or a targeting component, wherein at least one R^{LD} is a lipid moiety.
   275. A composition comprising a plurality of compounds having the structure of:
A^{c}-[-L^{LD}-(R^{LD})ₐ]_{b} or [(A^{c})ₐ-L^{LD}]_{b}-R^{LD}, or a salt thereof,
   wherein:
A^{c} is a biologically active agent;
a is 1-1000;
b is 1-1000;
each L^{LD} is independently a covalent bond or an optionally substituted, C₁-C₈₀ saturated or partially unsaturated aliphatic group, wherein one or more methylene units are optionally and independently replaced by T^{L}D or an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
each R^{LD} is independently an optionally substituted, C₁-C₈₀ saturated or partially unsaturated aliphatic group, wherein one or more methylene units are optionally and independently replaced by an optionally substituted group selected from C₁-C₆ alkylene, C₁-C₆ alkenylene, —CC, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
T^{LD} has the structure of:

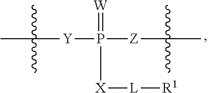

W is O, S or Se;
each of X, Y and Z is independently —O—, —S—, —N(-L-R′)—, or L;
L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene; and
each R is independently hydrogen, or an optionally substituted group selected from C₁-C₆ aliphatic, carbocyclyl, aryl, heteroaryl, and heterocyclyl.
   276. The composition of any one of embodiments 274-275, wherein A^{c} is an oligonucleotide chain ([H]_{b}-A^{c} is an oligonucleotide).
   277. The composition or method of any one of embodiments 1-273, wherein the composition is a composition of any one of embodiments 274-276.
   278. The composition or method of any one of embodiments 274-277, wherein the oligonucleotides or oligonucleotides have the structure of A^{c}-[-L^{LD}-(R^{LD})ₐ]_{b}.
   279. The composition or method of any one of embodiments 274-277, wherein the oligonucleotides or oligonucleotides have the structure of [(A^{c})ₐ-L^{LD}]_{b}-R^{LD}.
   280. The composition or method of any one of embodiments 274-279, wherein L^{LD}, R^{LD} combinations of L^{LD} and R^{LD}, or -[-L^{LD}-(R^{LD})ₐ]_{b} comprises one or more lipid moieties.
   281. The composition or method of any one of embodiments 274-279, wherein -[-L^{LD}-(R^{LD})ₐ]_{b} comprises one or more lipid moieties.
   282. The composition or method of any one of embodiments 274-280, wherein R^{LD} comprises one or more lipid moieties.
   283. The composition or method of any one of embodiments 274-279, wherein L^{LD}, R^{LD} combinations of L^{LD} and R^{LD}, or -[-L^{LD}-(R^{LD})ₐ]_{b} comprises one or more targeting components.
   284. The composition or method of any one of embodiments 274-279, wherein -[-L^{LD}-(R^{LD})ₐ]_{b} comprises one or more targeting components.
   285. The composition or method of any one of embodiments 274-280, wherein R^{LD} comprises one or more targeting components.
   286. The composition or method of any one of embodiments 274-285, wherein b is 1.
   287. The composition or method of any one of embodiments 274-286, wherein a is 1.
   288. The composition or method of any one of embodiments 274-287, wherein A^{c} comprises one or more modified base, sugar, or internucleotidic linkage moieties.
   289. The composition or method of any one of embodiments 274-288, wherein A^{c} comprises one or more chiral internucleotidic linkages.
   290. The composition or method of any one of embodiments 274-289, wherein A^{c} comprises one or more chiral internucleotidic linkages, and each chiral internucleotidic linkage of A^{c} is chirally controlled.
   291. The composition or method of any one of embodiments 274-290, wherein oligonucleotides having the structure of A^{c}-[-L^{LD}-(R^{LD})ₐ]_{b}, or [(A^{c})ₐ-L^{LD}]_{b}-R^{LD}, are of a particular type defined by the 1) base sequence; 2) pattern of backbone linkages; 3) pattern of backbone chiral centers; and 4) pattern of backbone phosphorus modifications of A.
   292. The composition or method of any one of embodiments 274-287, wherein A^{c} is the oligonucleotide chain of any one of the preceding embodiments.
   293. The composition or method of any one of embodiments 274-292, wherein A^{c} is an oligonucleotide of any one of the preceding embodiments, connecting to L^{LD} through a hydroxyl group of a sugar moiety (—O—).
   294. The composition or method of any one of embodiments 274-293, wherein A^{c} is an oligonucleotide of any one of the preceding embodiments, connecting to L^{LD} through its 5′-O—.
   295. The composition or method of any one of embodiments 274-292, wherein A^{c} is an oligonucleotide of any one of the preceding embodiments, connecting to L^{LD} through a nucleobase.
   296. The composition or method of any one of embodiments 274-292, wherein A^{c} is an oligonucleotide of any one of the preceding embodiments, connecting to L^{LD} through an internucleotidic linkage.
   297. The composition or method of any one of embodiments 274-294, wherein A^{c} is an oligonucleotide selected from any of the Tables and connected to L^{LD} and R^{LD} ([H]_{b}-A^{c} is an oligonucleotide selected from any of the Tables).
   298. The composition or method of any one of embodiments 274-294, wherein A^{c} is WV-887 connected to L^{LD} and R^{LD} ([H]_{b}-A^{c} is WV-887).
   299. The composition or method of any one of embodiments 274-294, wherein A^{c} is WV-892 connected to L^{LD} and R^{LD}.
   300. The composition or method of any one of embodiments 274-294, wherein A^{c} is WV-896 connected to L^{LD} and R^{LD}.
   301. The composition or method of any one of embodiments 274-294, wherein A^{c} is WV-1714 connected to L^{LD} and R^{LD}.
   302. The composition or method of any one of embodiments 274-294, wherein A^{c} is WV-2444 connected to L^{LD} and R^{LD}.
   303. The composition or method of any one of embodiments 274-294, wherein A^{c} is WV-2445 connected to L^{LD} and R^{LD}.
   304. The composition or method of any one of embodiments 274-294, wherein A^{c} is WV-2526 connected to L^{LD} and R^{LD}.
   305. The composition or method of any one of embodiments 274-294, wherein A^{c} is WV-2527 connected to L^{LD} and R^{LD}.
   306. The composition or method of any one of embodiments 274-294, wherein A^{c} is WV-2528 connected to L^{LD} and R^{LD}.
   307. The composition or method of any one of embodiments 274-294, wherein A^{c} is WV-2530 connected to L^{LD} and R^{LD}.
   308. The composition or method of any one of embodiments 274-294, wherein A^{c} is WV-2531 connected to L^{LD} and R^{LD}.
   309. The composition or method of any one of embodiments 274-294, wherein A^{c} is WV-2578 connected to L^{LD} and R^{LD}.
   310. The composition or method of any one of embodiments 274-294, wherein A^{c} is WV-2580 connected to L^{LD} and R^{LD}.
   311. The composition or method of any one of embodiments 274-294, wherein A^{c} is WV-2587 connected to L^{LD} and R^{LD}.
   312. The composition or method of any one of embodiments 274-294, wherein A^{c} is WV-3047 connected to L^{LD} and R^{LD}.
   313. The composition or method of any one of embodiments 274-294, wherein A^{c} is WV-3152 connected to L^{LD} and R^{LD}.
   314. The composition or method of any one of embodiments 274-294, wherein A^{c} is WV-3472 connected to L^{LD} and R^{LD}.
   315. The composition or method of any one of embodiments 274-294, wherein A^{c} is WV-3473 connected to L^{LD} and R^{LD}.
   316. The composition or method of any one of embodiments 274-294, wherein A^{c} is WV-3507 connected to L^{LD} and R^{LD}.
   317. The composition or method of any one of embodiments 274-294, wherein A^{c} is WV-3508 connected to L^{LD} and R^{LD}.
   318. The composition or method of any one of embodiments 274-294, wherein A^{c} is WV-3509 connected to L^{LD} and R^{LD}.
   319. The composition or method of any one of embodiments 274-294, wherein A^{c} is WV-3510 connected to L^{LD} and R^{LD}.
   320. The composition or method of any one of embodiments 274-294, wherein A^{c} is WV-3511 connected to L^{LD} and R^{LD}.
   321. The composition or method of any one of embodiments 274-294, wherein A^{c} is WV-3512 connected to L^{LD} and R^{LD}.
   322. The composition or method of any one of embodiments 274-294, wherein A^{c} is WV-3513 connected to L^{LD} and R^{LD}.
   323. The composition or method of any one of embodiments 274-294, wherein A^{c} is WV-3514 connected to L^{LD} and R^{LD}.
   324. The composition or method of any one of embodiments 274-294, wherein A^{c} is WV-3515 connected to L^{LD} and R^{LD}.
   325. The composition or method of any one of embodiments 274-324, wherein L^{LD} is an optionally substituted, C₁-C₁₀ saturated or partially unsaturated aliphatic group, wherein one or more methylene units are optionally and independently replaced by T^{LD} or an optionally substituted group selected from C₁-C₆ alkylene, C₁-C₆ alkenylene, —C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—.
   326. The composition or method of any one of embodiments 274-324, wherein L^{LD} is an optionally substituted, C₁-C₁₀ saturated or partially unsaturated aliphatic group, wherein one or more methylene units are optionally and independently replaced by an optionally substituted group selected from C₁-C₆ alkylene, C₁-C₆ alkenylene, —C≡C—, -Cy-, —O—, —S—, —N(R′)—, —C(O)—, —C(O)N(R′)—, —N(R′)C(O)N(R′)—, —OC(O)N(R′)—, —S(O)—, —S(O)₂—, —S(O)₂N(R′)—, and —C(O)O—, or T^{L}D wherein W is O or S, each of Y and Z is independently —O—, —S—, or -L-.
   327. The composition or method of any one of embodiments 274-324, wherein L^{L}D is an optionally substituted, C₁-C₁₀ saturated or partially unsaturated aliphatic group, wherein one or more methylene units are optionally and independently replaced by an optionally substituted group selected from C₁-C₆ alkylene, C₁-C₆ alkenylene, —C≡C—, -Cy-, —O—, —S—, —N(R′)—, —C(O)—, —C(O)N(R′)—, —N(R′)C(O)N(R′)—, —OC(O)N(R′)—, —S(O)—, —S(O)₂—, —S(O)₂N(R′)—, and —C(O)O—, or T^{LD} wherein W is O or S, each of X and Y is independently —O—, —S—, or -L-, and Z is a covalent bond.
   328. The composition or method of any one of embodiments 274-326, wherein L^{LD} connects to a hydroxyl group of A^{c}.
   329. The composition or method of any one of embodiments 274-326, wherein L^{LD} connects to 5′-OH of A^{c}.
   330. The composition or method of any one of embodiments 274-326, wherein L^{LD} connects to 3′-OH of A^{c}.
   331. The composition or method of any one of the preceding embodiments, wherein each R′ is independently —R, —C(O)R, —CO₂R, or —SO₂R, or:

two R′ are taken together with their intervening atoms to form an optionally substituted C₃-C₁₄ monocyclic, bicyclic or polycyclic aryl, carbocyclic, heterocyclic, or heteroaryl ring having 0-10 heteroatoms.

332. The composition or method of any one of the preceding embodiments, wherein -Cy- is an optionally substituted bivalent ring selected from C₃-C₁₄ monocyclic, bicyclic or polycyclic carbocyclylene, arylene, heteroarylene, and heterocyclylene having 0-10 heteroatoms.
333. The composition or method of any one of the preceding embodiments, wherein each R is independently hydrogen, or an optionally substituted group selected from C₁-C₆ aliphatic, and C₃-C₁₄ monocyclic, bicyclic or polycyclic aryl, carbocyclic, heterocyclic, or heteroaryl ring having 0-10 heteroatoms.
334. The composition or method of any one of embodiments 274-326, wherein L^{LD} is T^{LD}.
335. The composition or method of any one of embodiments 274-326, wherein L^{LD} is —NH—(CH₂)₆-T^{LD}-.
336. The composition or method of any one of embodiments 274-326, wherein L^{LD} is —C(O)—NH—(CH₂)₆-T^{LD}-.
337. The composition or method of embodiment 336, wherein —C(O)— is connected to —R^{LD}.
338. The composition or method of any one of embodiments 274-337, wherein T^{LD} is connected to 5′-O— or 3′-O— of A^{c}.
339. The composition or method of any one of embodiments 274-338, wherein T^{LD} is connected to 5′-O— of A^{c}.
340. The composition or method of any one of embodiments 274-338, wherein T^{LD} is connected to 3′-O— of A^{c}.
341. The composition or method of any one of embodiments 274-340, wherein T^{LD} forms a phosphorothioate linkage with 5′-O— or 3′-O— of A^{c}.
342. The composition or method of embodiment 341, wherein a phosphorothioate linkage is chirally controlled and is Sp.
343. The composition or method of embodiment 341, wherein a phosphorothioate linkage is chirally controlled and is Rp.
344. The composition or method of any one of embodiments 274-340, wherein T^{LD} forms a phosphate linkage with 5′-O— or 3′-O— of A^{c}.
345. The composition or method of any one of embodiments 274-324, wherein L^{LD} is a covalent bond.
346. The composition or method of any one of the preceding embodiments, R^{LD} is an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—.
347. The composition or method of any one of the preceding embodiments, wherein R^{LD} is an optionally substituted, C₁₀-C₈₀ saturated or partially unsaturated aliphatic group, wherein one or more methylene units are optionally and independently replaced by —C(O)—.
348. The composition or method of any one of the preceding embodiments, wherein R^{LD} is an optionally substituted, C₁₀-C₆₀ saturated or partially unsaturated aliphatic group, wherein one or more methylene units are optionally and independently replaced by —C(O)—.
349. The composition or method of any one of the preceding embodiments, wherein R^{LD} is an optionally substituted, C₁₀-C₄₀ saturated or partially unsaturated aliphatic group, wherein one or more methylene units are optionally and independently replaced by —C(O)—.
350. The composition or method of any one of the preceding embodiments, wherein R^{LD} comprises 10, 11, 12, 13, 14, 15, 16, 17, 18, 19, 20, 21, 22, 23, 24, 25, 26, 27, 28, 29, 30 or more carbon atoms.
351. The composition or method of any one of the preceding embodiments, wherein at least one R^{LD} comprises or is a targeting component.
352. The composition or method of any one of the preceding embodiments, wherein at least one R^{LD} is a targeting component.
353. The composition or method of any one of the preceding embodiments, wherein at least one R^{LD} comprises a lipid moiety.
354. The composition or method of any one of the preceding embodiments, wherein at least one R^{LD} is a lipid moiety.
355. The composition or method of any one of the preceding embodiments, wherein R^{LD} is an optionally substituted, C₁₀-C₈₀ saturated or partially unsaturated aliphatic group.
356. The composition or method of any one of the preceding embodiments, wherein R^{LD} is an optionally substituted, C₁₀-C₆₀ saturated or partially unsaturated aliphatic group.
357. The composition or method of any one of the preceding embodiments, wherein R^{LD} is an optionally substituted, C₁₀-C₄₀ saturated or partially unsaturated aliphatic group.
358. The composition or method of any one of the preceding embodiments, wherein R^{LD} is unsubstituted linear or branched C₁₀-C₈₀ aliphatic group.
359. The composition or method of any one of the preceding embodiments, wherein R^{LD} is unsubstituted linear or branched C₁₀-C₆₀ aliphatic group.
360. The composition or method of any one of the preceding embodiments, wherein R^{LD} is unsubstituted linear or branched C₁₀-C₄₀ aliphatic group.
361. The composition or method of any one of embodiments 274-360, wherein R^{LD} is palmityl.
362. The composition or method of any one of the preceding embodiments 274-360, wherein R^{LD} is

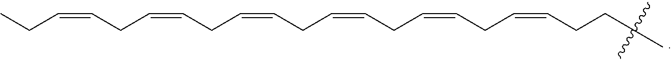 [SMILES: *CC/C=C\C/C=C\C/C=C\C/C=C\C/C=C\C/C=C\CC]

363. The composition or method of any one of embodiments 274-360, wherein R^{LD} is lauryl.
364. The composition or method of any one of embodiments 274-360, wherein R^{LD} is myristyl.
365. The composition or method of any one of embodiments 274-360, wherein R^{LD} is stearyl.
366. The composition or method of any one of embodiments 274-360, wherein R^{LD} is

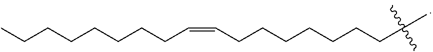 [SMILES: *CCCCCCC/C=C\CCCCCCCC]

367. The composition or method of any one of embodiments 274-360, wherein R^{LD} is

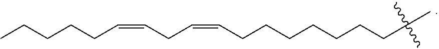 [SMILES: *CCCCCCC/C=C\C/C=C\CCCCC]

368. The composition or method of any one of embodiments 274-360, wherein R^{LD} is

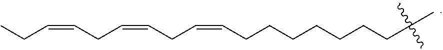 [SMILES: *CCCCCCC/C=C\C/C=C\C/C=C\CC]

369. The composition or method of any one of embodiments 274-360, wherein R^{LD} is

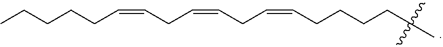 [SMILES: *CCCC/C=C\C/C=C\C/C=C\CCCCC]

370. The composition or method of any one of embodiments 274-360, wherein R^{LD} is

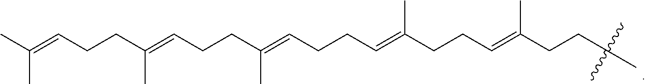 [SMILES: *CC/C(C)=C/CC/C(C)=C/CC/C=C(\C)CC/C=C(\C)CCC=C(C)C]

371. The composition or method of any one of embodiments 274-360, wherein R^{LD} is

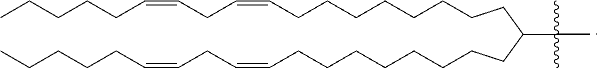 [SMILES: *C(CCCCCCCC/C=C\C/C=C\CCCCC)CCCCCCCC/C=C\C/C=C\CCCCC]

372. The composition or method of any one of embodiments 274-354, wherein R^{LD} is

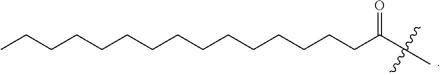 [SMILES: *C(=O)CCCCCCCCCCCCCCC]

373. The composition or method of any one of embodiments 274-354, wherein R^{LD} is

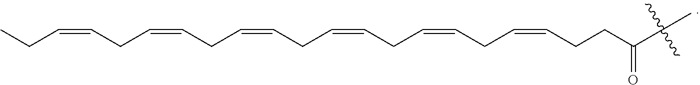 [SMILES: *C(=O)CC/C=C\C/C=C\C/C=C\C/C=C\C/C=C\C/C=C\CC]

374. The composition or method of any one of embodiments 274-354, wherein R^{LD} is

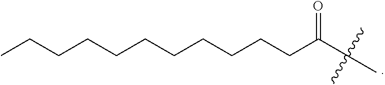 [SMILES: *C(=O)CCCCCCCCCCC]

375. The composition or method of any one of embodiments 274-354, wherein R^{LD} is

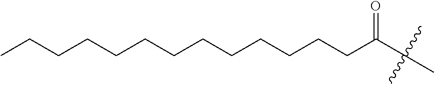 [SMILES: *C(=O)CCCCCCCCCCCCC]

376. The composition or method of any one of embodiments 274-354, wherein R^{LD} is

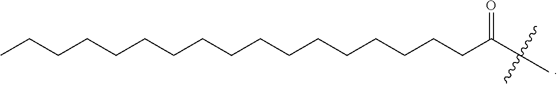 [SMILES: *C(=O)CCCCCCCCCCCCCCCCC]

377. The composition or method of any one of embodiments 274-354, wherein R^{LD} is

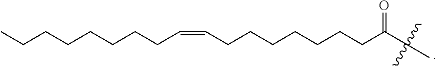 [SMILES: *C(=O)CCCCCCC/C=C\CCCCCCCC]

378. The composition or method of any one of embodiments 274-354, wherein R^{LD} is

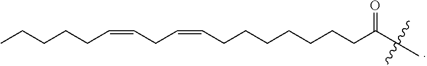 [SMILES: *C(=O)CCCCCCC/C=C\C/C=C\CCCCC]

379. The composition or method of any one of embodiments 274-354, wherein R^{LD} is

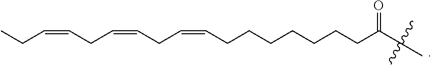 [SMILES: *C(=O)CCCCCCC/C=C\C/C=C\C/C=C\CC]

380. The composition or method of any one of embodiments 274-354, wherein R^{LD} is

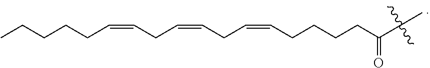 [SMILES: *C(=O)CCCC/C=C\C/C=C\C/C=C\CCCCC]

381. The composition or method of any one of embodiments 274-354, wherein R^{LD} is

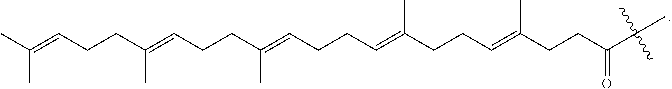 [SMILES: *C(=O)CC/C(C)=C/CC/C(C)=C/CC/C=C(\C)CC/C=C(\C)CCC=C(C)C]

382. The composition or method of any one of embodiments 274-354, wherein R^{LD} is

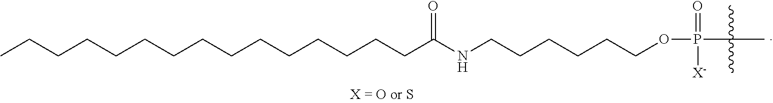

383. The composition or method of any one of embodiments 274-354, wherein R^{LD} is

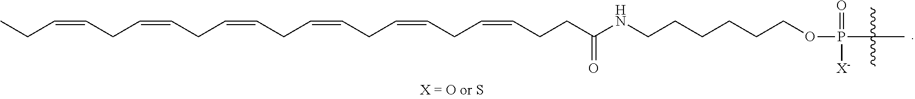

384. The composition or method of any one of embodiments 274-354, wherein R^{LD} is

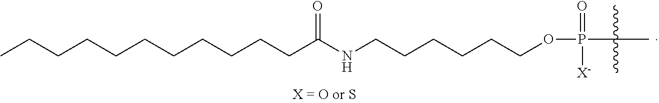

385. The composition or method of any one of embodiments 274-354, wherein R^{LD} is

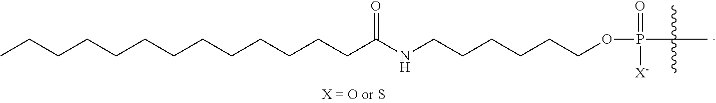

386. The composition or method of any one of embodiments 274-354, wherein R^{LD} is

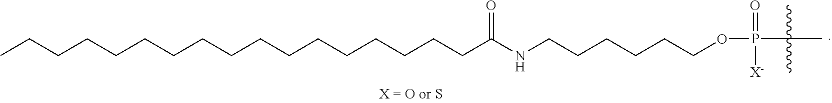

387. The composition or method of any one of embodiments 274-354, wherein R^{LD} is

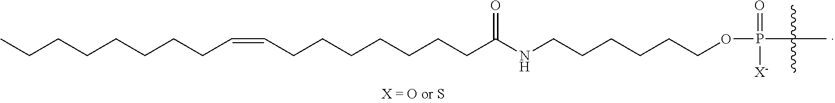

388. The composition or method of any one of embodiments 274-354, wherein R^{LD} is

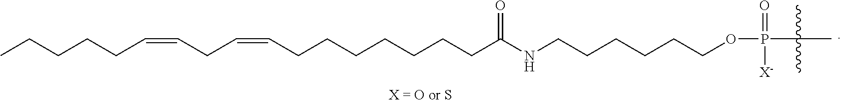

389. The composition or method of any one of embodiments 274-354, wherein R^{LD} is

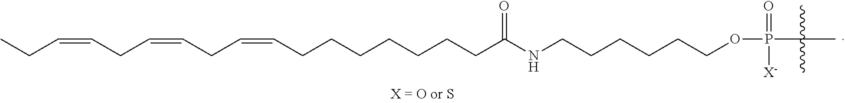

390. The composition or method of any one of embodiments 274-354, wherein R^{LD} is

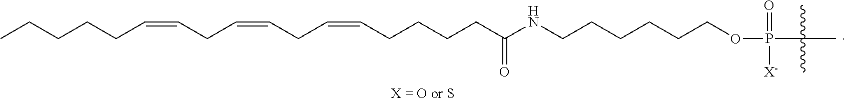

391. The composition or method of any one of embodiments 274-354, wherein R^{LD} is

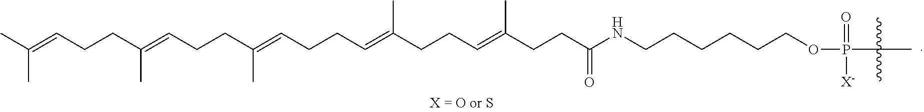

392. The composition or method of any one of embodiments 274-354, wherein R^{LD} is

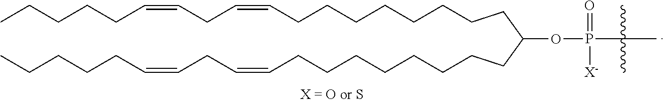

393. The composition or method of any one of embodiments 274-354, wherein R^{LD} is

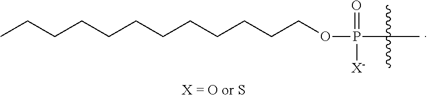

394. The composition or method of any one of embodiments 274-354, wherein R^{LD} is

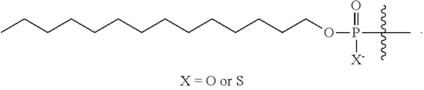

395. The composition or method of any one of embodiments 274-354, wherein R^{LD} is

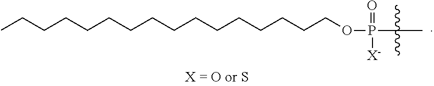

396. The composition or method of any one of embodiments 274-354, wherein R^{LD} is

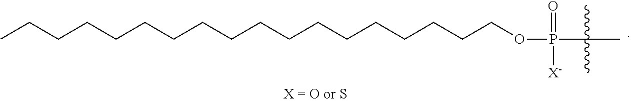

397. The composition or method of any one of embodiments 274-354, wherein -[-L^{LD}-(R^{LD})ₐ]_{b} is

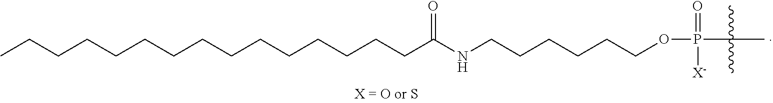

398. The composition or method of any one of embodiments 274-354, wherein -[-L^{LD}-(R^{LD})ₐ]_{b} is

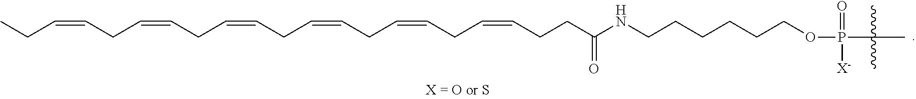

399. The composition or method of any one of embodiments 274-354, wherein

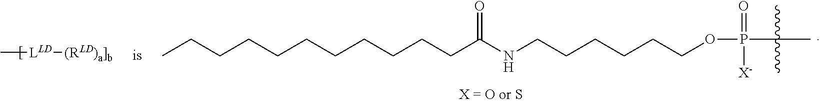

400. The composition or method of any one of embodiments 274-354, wherein -[-L^{LD}-(R^{LD})ₐ]_{b} is

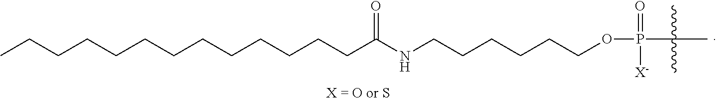

401. The composition or method of any one of embodiments 274-354, wherein -[-L^{LD}-(R^{LD})ₐ]_{b} is

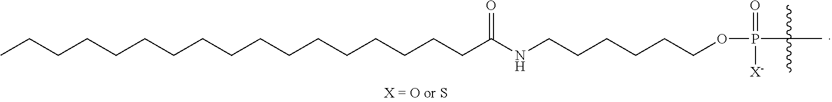

402. The composition or method of any one of embodiments 274-354, wherein -[-L^{LD}-(R^{LD})ₐ]_{b} is

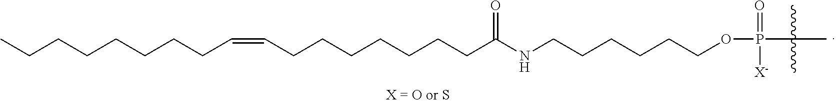

403. The composition or method of any one of embodiments 274-354, wherein -[-L^{LD}-(R^{LD})ₐ]_{b} is

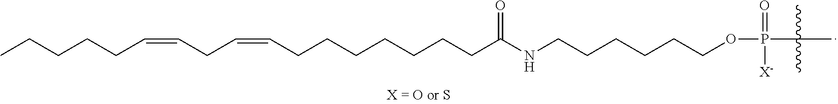

404. The composition or method of any one of embodiments 274-354, wherein -[-L^{LD}-(R^{LD})ₐ]_{b} is

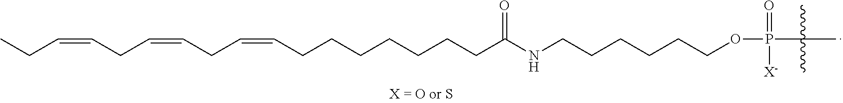

405. The composition or method of any one of embodiments 274-354, wherein -[-L^{LD}-(R^{LD})ₐ]_{b} is

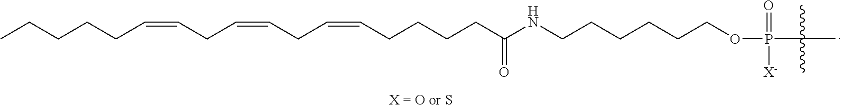

406. The composition or method of any one of embodiments 274-354, wherein -[-L^{LD}-(R^{LD})ₐ]_{b} is

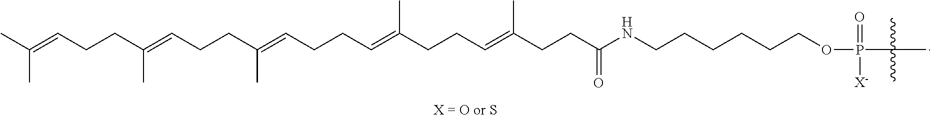

407. The composition or method of any one of embodiments 274-354, wherein -[-L^{LD}-(R^{LD})ₐ]_{b} is

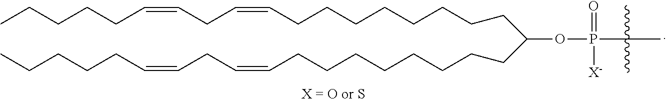

408. The composition or method of any one of embodiments 274-354, wherein -[-L^{LD}(R^{LD})ₐ]_{b} is

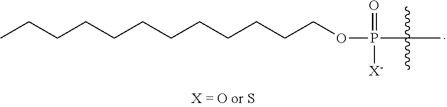

409. The composition or method of any one of embodiments 274-354, wherein -[-L^{LD}-(R^{LD})ₐ]_{b} is

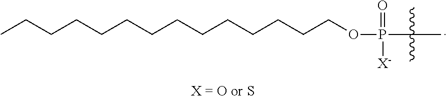

410. The composition or method of any one of embodiments 274-354, wherein -[-L^{LD}-(R^{LD})ₐ]_{b} is

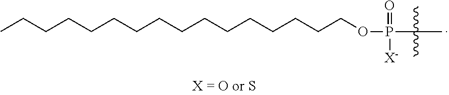

411. The composition or method of any one of embodiments 274-354, wherein -[-L^{LD}-(R^{LD})ₐ]_{b} is

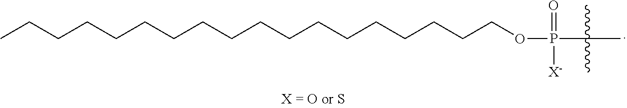

412. The composition or method of any one of embodiments 382-411, wherein X is O.
413. The composition or method of any one of embodiments 382-411, wherein X is S.
414. The composition or method of embodiment 412, wherein —O—P(O)(X⁻)— connects to 5′-O— of A^{c} to form a phosphate linkage.
415. The composition or method of embodiment 412, wherein —O—P(O)(X⁻)— connects to 3′-O— of A^{c} to form a phosphate linkage.
416. The composition or method of embodiment 413, wherein —O—P(O)(X⁻)— connects to 5′-O— of A^{c} to form a phosphorothioate linkage.
417. The composition or method of embodiment 413, wherein —O—P(O)(X⁻)— connects to 3′-O— of A^{c} to form a phosphorothioate linkage.
418. The composition or method of embodiment 416 or 417, wherein the phosphorothioate linkage is chirally controlled.
419. The composition or method of embodiment 416 or 417, wherein the phosphorothioate linkage is chirally controlled and is Sp.
420. The composition or method of embodiment 416 or 417, wherein the phosphorothioate linkage is chirally controlled and is Rp.
421. The composition or method of any one of the preceding embodiments, wherein at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%0, 90%, 91%, 92%, 93%, 94%, 95%, 96%, 97%, 97% or 99% of the oligonucleotides that have the base sequence of the particular oligonucleotide type, defined by 1) base sequence; 2) pattern of backbone linkages; 3) pattern of backbone chiral centers; and 4) pattern of backbone phosphorus modifications, are oligonucleotides of the particular oligonucleotide type.
422. The composition or method of any one of the preceding embodiments, wherein at least 10% of the oligonucleotides that have the base sequence of the particular oligonucleotide type, defined by 1) base sequence; 2) pattern of backbone linkages; 3) pattern of backbone chiral centers; and 4) pattern of backbone phosphorus modifications, are oligonucleotides of the particular oligonucleotide type.
423. The composition or method of any one of the preceding embodiments, wherein at least 0.5%, 1%, 2%, 3%, 4%, 5%, 6%, 7%, 8%, 9%, 10%, 20%, 30%, 40%, 50%, 60%, 70%, 75%, 80%, 81%, 82%, 83%, 84%, 85%, 86%, 87%, 88%, 89%0, 90%, 91%, 92%, 93%, 94%, 95%, 96%, 97%, 97% or 99% of the oligonucleotides that have the base sequence, pattern of backbone linkages, and pattern of backbone phosphorus modifications of the particular oligonucleotide type, defined by 1) base sequence; 2) pattern of backbone linkages; 3) pattern of backbone chiral centers; and 4) pattern of backbone phosphorus modifications, are oligonucleotides of the particular oligonucleotide type.
424. The composition or method of any one of the preceding embodiments, wherein at least 10% of the oligonucleotides that have the base sequence, pattern of backbone linkages, and pattern of backbone phosphorus modifications of the particular oligonucleotide type, defined by 1) base sequence; 2) pattern of backbone linkages; 3) pattern of backbone chiral centers; and 4) pattern of backbone phosphorus modifications, are oligonucleotides of the particular oligonucleotide type.
425. The composition or method of any one of the preceding embodiments, wherein the composition is a pharmaceutical composition comprising one or more pharmaceutically acceptable salts of the oligonucleotides.
426. The composition or method of any one of the preceding embodiments, wherein the composition is a pharmaceutical composition comprising one or more sodium salts of the oligonucleotides.
427. The composition or method of any one of the proceeding embodiments, wherein the composition further comprises one or more other therapeutic agents.
428. A method of generating a set of spliced products from a target transcript, the method comprising steps of:

contacting a splicing system containing the target transcript with an oligonucleotide composition of one of the previous embodiments in an amount, for a time, and under conditions sufficient for a set of spliced products to be generated that is different from a set generated under reference conditions selected from the group consisting of absence of the composition, presence of a reference composition, and combinations thereof.

429. A method for treating a disease, comprising administering to a subject a composition of any one of the preceding embodiments.
430. The method of any one of the preceding embodiments, wherein the disease is Duchenne muscular dystrophy.
431. A method of identifying and/or characterizing an oligonucleotide composition, the method comprising steps of:

providing at least one composition of any one of the preceding embodiments;

assessing splicing pattern of a transcript relative to a reference composition.

432. An oligonucleotide described in any one of the preceding embodiments or a salt thereof.

### EXAMPLES

Non-limiting examples are provided below. A person of ordinary skill in the art appreciates that other compositions and methods can similarly be prepared and performed in accordance with the present disclosure.

### Example 1. Synthesis of turbinaric acid and 2-cyanoethyl ((6Z,9Z,28Z,31Z)-heptatriaconta-6,9,28,31-tetraen-19-yl) diisopropylphosphoramidite

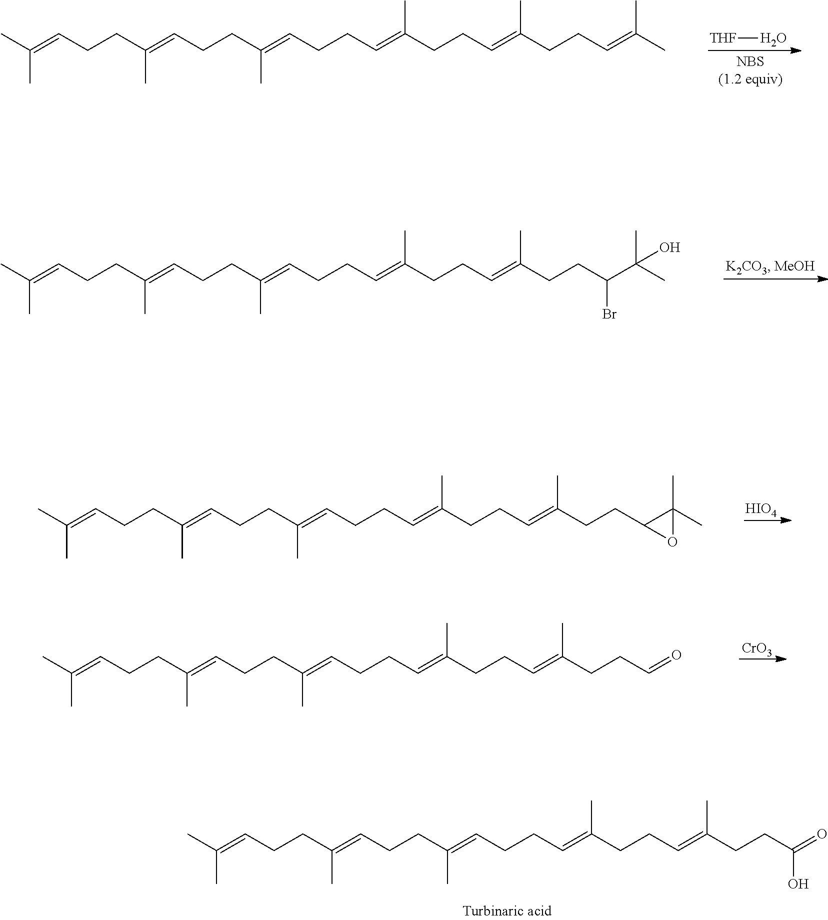

Synthesis of Turbinaric Acid: (4E,8E,12E,16E)-4,8,13,17,21-pentamethyldocosa-4,8,12,16,20-pentaenoic acid. First-step, synthesis of 2-hydroxy-3-bromosqualene. To a solution of squalene (30.03 g, 73.1 mmol) in THE (210 mL), water (35 mL) was added and then a small amount of THE was added dropwise to obtain a clear solution under Argon. N-bromosuccinimide (15.62 g, 88 mmol) was added portion-wise at 0° C. and the reaction mixture was stirred at 0° C. for 30 minutes and at room temperature for 3 hrs. The solvent was removed under reduced pressure, and brine (500 mL) was added and extracted with EtOA (100 mL×5). The organic layer was dried over anhydrous sodium sulfate and concentrated to give a residue, which was purified by ISCO (220 g gold silica gel cartridge) eluting with hexane to 50% EtOAc in hexane (product was come out at 10-20% EtOAc in hexane) to give 2-hydroxy-3-bromosqualene (9.92 g, 19.54 mmol, 26.7% yield) as a pale yellowish oil. ¹H NMR (400 MHz, Chloroform-d) δ 5.24-5.05 (m, 5H), 3.98 (dd, J=11.3, 1.9 Hz, 1H), 2.35-2.32 (m, 1H), 2.16-1.90 (m, 18H), 1.85-1.70 (m, 1H), 1.67 (d, J=1.4 Hz, 3H), 1.60 (bs, 15H), 1.34 (s, 3H), 1.32 (s, 3H). MS (ESI), 551.1 and 553.3 (M+HCOO)⁻.

Second step, synthesis of 2,2-dimethyl-3-((3E,7E,11E,15E)-3,7,12,16,20-pentamethylhenicosa-3,7,11,15,19-pentaen-1-yl)oxirane. To a solution of 2-hydroxy-3-bromosqualene (9.72 g, 19.15 mmol) in MeOH (360 mL), K₂CO₃ (5.29 g, 38.3 mmol) was added and the reaction mixture was stirred at room temperature for 2 hrs, filtered and then concentrated under reduced pressure. Then 300 mL EtOAc was added, and filtered, concentrated to give 2,3-oxidosqualene (8.38 g, 19.64 mmol, 100% yield) a colorless oil. ¹H NMR (400 MHz, Chloroform-d) δ 5.20-5.04 (m, 5H), 2.70 (t, J=7.0 Hz, 1H), 2.20-1.95 (m, 20H), 1.67 (s, 3H), 1.61 (s, 3H), 1.59 (bs, 15H), 1.29 (s, 3H), 1.25 (s, 3H).

Third step, synthesis of (4E,8E,12E,16E)-4,8,13,17,21-pentamethyldocosa-4,8,12,16,20-pentaenal. To a solution of periodic acid (7.79 g, 34.2 mmol) in water (28 mL) at 0° C., a solution of 2,3-oxidosqualene (8.10 g, 18.98 mmol) in dioxane (65 mL) was added. The reaction mixture was stirred at room temperature for 2 hrs. Water (150 mL) was added and extracted with EtOAc (3×100 mL). The organic layer are dried over anhydrous sodium sulfate and concentrated under reduced pressure to give a residue, which was purified by ISCO (120 g gold silica gel cartridge) eluting with hexane to 10% EtOAc in hexane (product come out at 5-7% EtOAc in hexane to give (4E,8E,12E,16E)-4,8,13,17,21-pentamethyldocosa-4,8,12,16,20-pentaenal (5.80 g, 15.08 mmol, 79% yield) as a colorless oil. ¹H NMR (400 MHz, Chloroform-d) δ 9.74 (t, J=2.0 Hz, 1H), 5.18-5.04 (m, 5H), 2.50 (td, J=7.5, 2.0 Hz, 2H), 2.31 (t, J=7.5 Hz, 2H), 2.13-1.92 (m, 16H), 1.67 (s, 3H), 1.61 (s, 3H), 1.59 (bs, 12H).

Fourth Step, synthesis of turbinaric Acid. Sulfuric acid (8.2 mL) followed by sodium dichromate dihydrate (4.42 g, 14.82 mmol) was added to HPLC water (80 mL) at 0° C. The above chromic acid solution was added dropwise to a solution of (4Z,8Z,12E,16E)-4,8,13,17,21-pentamethyldocosa-4,8,12,16,20-pentaenal (5.70 g, 14.82 mmol) in ethyl ether (115 mL) at 0° C. The reaction mixture was stirred at 0° C. for 2 hrs. After 2 hrs, TLC showed the reaction was complete (3:1 hexane/EtOAc). The reaction mixture was diluted with EtOAc (300 mL), washed with brine (100 mL×4), dried over a hydrous, concentrated to give a residue, which was purified by ISCO (80 g silica gel cartridge) eluting with DCM to 5% MeOH in DCM to give turbinaric acid as a colorless oil (5.00 g, 84% yield). ¹H NMR (400 MHz, Chloroform-d) δ 5.18-5.07 (m, 5H), 2.44 (t, J=6.5 Hz, 2H), 2.30 (t, J=7.7 Hz, 2H), 2.13-1.93 (m, 16H), 1.67 (s, 3H), 1.59 (bs, 15H); MS (ESI), 399.3 (M−H)⁻.

### Example 2. Synthesis of 2-cyanoethyl ((6Z,9Z,28Z,31Z)-heptatriaconta-6,9,28,31-tetraen-19-yl) diisopropylphosphoramidite

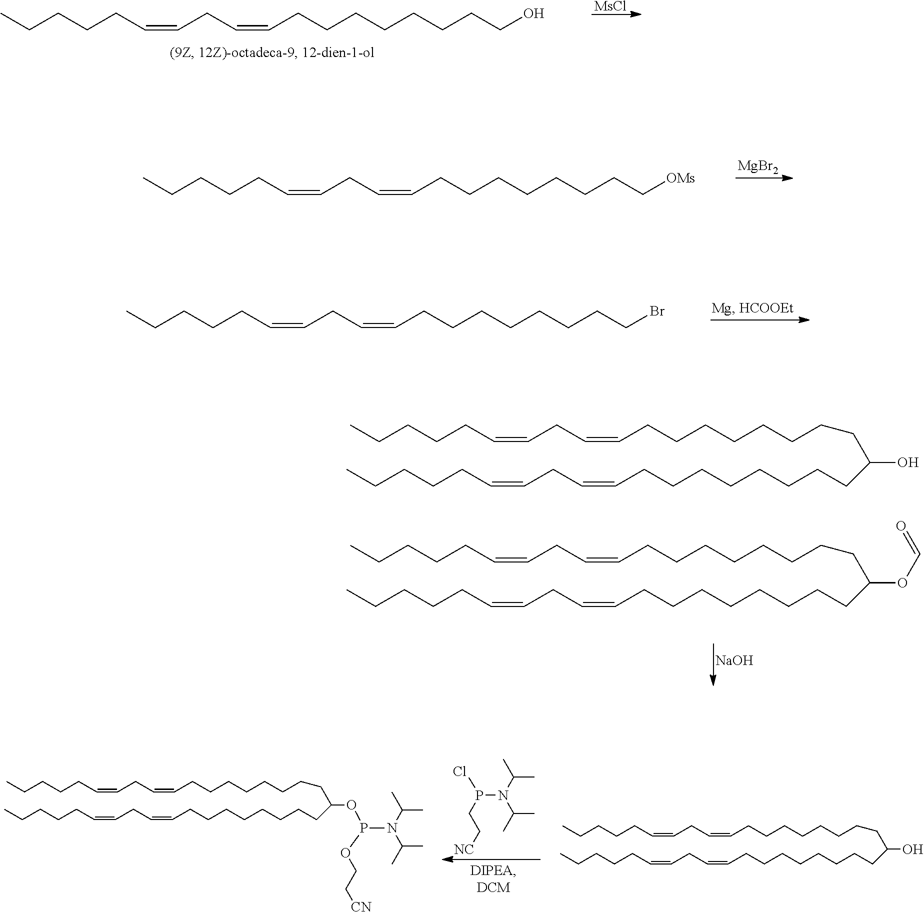

Synthesis of 2-cyanoethyl ((6Z,9Z,28Z,31Z)-heptatriaconta-6,9,28,31-tetraen-19-yl) diisopropylphosphoramidite. First-step, synthesis of (9Z,12Z)-octadeca-9,12-dien-1-yl methanesulfonate (or linoleyl methanesulfonate). To a solution of linoleyl alcohol (23.31 ml, 75 mmol) and triethylamine (13.60 ml, 98 mmol) in DCM (150 mL) at 0° C. was added methanesulfonyl chloride (6.39 ml, 83 mmol) dropwise. The reaction mixture was stirred at 0° C. for 30 minutes and at room temperature for 3 hrs. The reaction mixture was diluted with DCM (200 mL), washed with water, sat sodium bicarbonate and brine and dried over a hydrous sodium sulfate. Solvent was concentrated to give linoleyl methanesulfonate (26.17 g, 100% yield) as an yellowish oil. Without further purification, directly use for next step. ¹H NMR (500 MHz, Chloroform-d) δ 5.30-5.41 (m, 4H), 4.22 (t, J=6.6 Hz, 2H), 2.99 (s, 3H), 2.77 (t, J=6.7 Hz, 2H), 2.05 (q, J=6.9 Hz, 4H), 1.74 (p, J=6.7 Hz, 2H), 1.43-1.25 (m, 16H), 0.89 (t, J=6.7 Hz, 3H).

Second-step, synthesis of linoleyl bromide. To a solution of linoleyl methanesulfonate (26 g, 75 mmol) in ether (800 mL) was added Magnesium bromide ethyl etherate (58.5 g, 226 mmol) under Argon. The reaction mixture was stirred at room temperature for 2 hrs. TLC showed the reaction was not completed. Additional magnesium bromide ethyl etherate (14.5 g) was added the reaction mixture and the reaction mixture was stirred at room temperature for 22 hrs. TLC showed the reaction was complete (9/1 hexane/EtOAc). The reaction mixture was filtered, washed with ether (200 mL), hexane (100 mL), concentrated under reduced pressure to give a residue, which was purified by ISCO (200 g gold silica gel cartridge) eluted with hexane to 10% EtOAc in hexane to give linoleyl bromide (22.8 g, 69.2 mmol, 92% yield) as a colorless oil. ¹H NMR (500 MHz, Chloroform-d) δ 5.42-5.31 (m, 4H), 3.41 (t, J=6.9 Hz, 2H), 2.77 (t, J=6.6 Hz, 2H), 2.05 (q, J=6.9 Hz, 4H), 1.85 (p, J=6.9 Hz, 2H), 1.43-1.25 (m, 16H), 0.89 (t, J=6.8 Hz, 3H).

Third-step, synthesis of dilinoleyl methanol. To a suspension of Mg (0.897 g, 36.9 mmol) and ether (20 mL) in RB flask was added linoleyl bromide (10.0 g, 30.4 mmol) in ether (25 mL) dropwise while keeping the reaction under gentle reflux by cooling the RB flask in water. The reaction mixture was stirred at 35° C. for 1 hour. To the above reaction mixture at 0° C. was added ethyl formate (1.013 g, 13.68 mmol) in ether (30 mL) dropwise for 10 minutes and the reaction mixture was stirred at room temperature for 1.5 hrs. The reaction mixture was cooled in ice bath, quenched with water (30 mL), treated with 10% H₂SO₄ (150 mL) until the solution became homogeneous and the layer was separated. The aqueous layer was extracted with ether (200 mL×2). The solvent was evaporated under reduced pressure to give a residue, which was re-dissolved in THE (50 mL) and 1 N NaOH (30 mL). The reaction mixture was stirred at 40° C. for 5 hrs. TLC showed the reaction was not complete. 1.5 g NaOH was added to the reaction mixture and the reaction mixture was continually stirred at 40° C. for overnight. The reaction mixture was extracted with ether (2×), dried over a hydrous sodium sulfate, concentrated to give a residue, which was purified by ISCO (120 g gold silica gel cartridge) eluting with hexane to 10% EtOAc in hexane to give dilinoleyl methanol (5.16 g, 9.76 mmol, 71.3% yield) as a colorless oil. ¹H NMR (500 MHz, Chloroform-d) δ 5.41-5.30 (m, 8H), 3.58 (s, 1H), 2.77 (t, J=6.7 Hz, 4H), 2.05 (q, J=6.9 Hz, 8H), 1.49-1.25 (m, 40H), 0.89 (t, J=6.8 Hz, 6H).

Fourth-step, 2-cyanoethyl ((6Z,9Z,28Z,31Z)-heptatriaconta-6,9,28,31-tetraen-19-yl) diisopropylphosphoramidite. To a solution of dilinoleyl methanol (2.5 g, 4.73 mmol) in anhdrous dichloromethane (30 mL) at room temperature was added DIPEA (4.12 ml, 23.63 mmol) and 3-(chloro(diisopropylamino)phosphino)propanenitrile (1.180 ml, 5.67 mmol). The reaction mixture was stirred at room temperature for 2 hours. The reaction mixture was added EtOAc (300 mL), washed with sat sodium bicarbonate, dried over a hydrous sodium sulfate, and concentrated under reduced pressure to give a residue, which was purified by ISCO (40 g gold silica gel cartridge) eluting with hexane to 5% EtOAc in hexane containing 5% TEA to give 2-cyanoethyl (6Z,9Z,28Z,31Z)-heptatriaconta-6,9,28,31-tetraen-19-yl diisopropylphosphoramidite (2.97 g, 4.07 mmol, 86% yield) as a colorless oil. ¹H NMR (500 MHz, Chloroform-d) δ 5.30-5.41 (m, 8H), 3.85-3.72 (m, 3H), 3.59 (dp, J=10.2, 6.8 Hz, 2H), 2.77 (t, J=6.8 Hz, 4H), 2.61 (t, J=6.6 Hz, 2H), 2.05 (q, J=7.1 Hz, 8H), 1.60-1.46 (m, 4H), 1.42-1.27 (m, 36H), 1.18 (dd, J=6.8, 3.0 Hz, 12H), 0.89 (t, J=6.8 Hz, 6H). ³¹P NMR (202 MHz, Chloroform-d) δ 147.68.

### Example 3. Lipid Conjugation of WV-942 Amino Linker

General route for conjugation of lipids with C₆-amino linked WV-942 (SEQ ID NO: 2423) is exemplified in the following Scheme (Scheme 1).

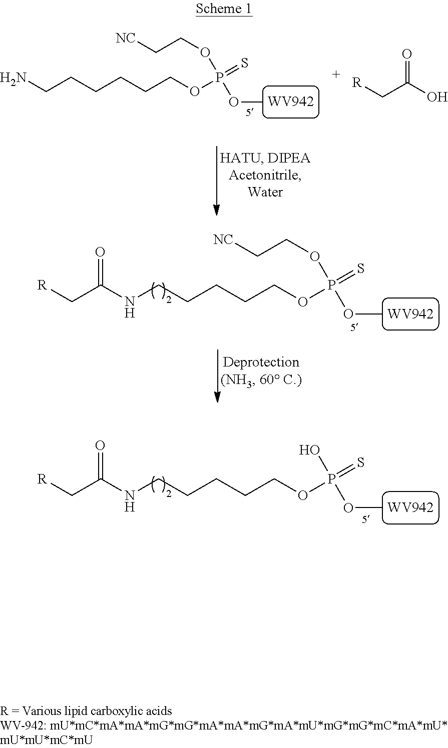

Structures of various lipid carboxylic acids and alcohols used for conjugation is depicted below:

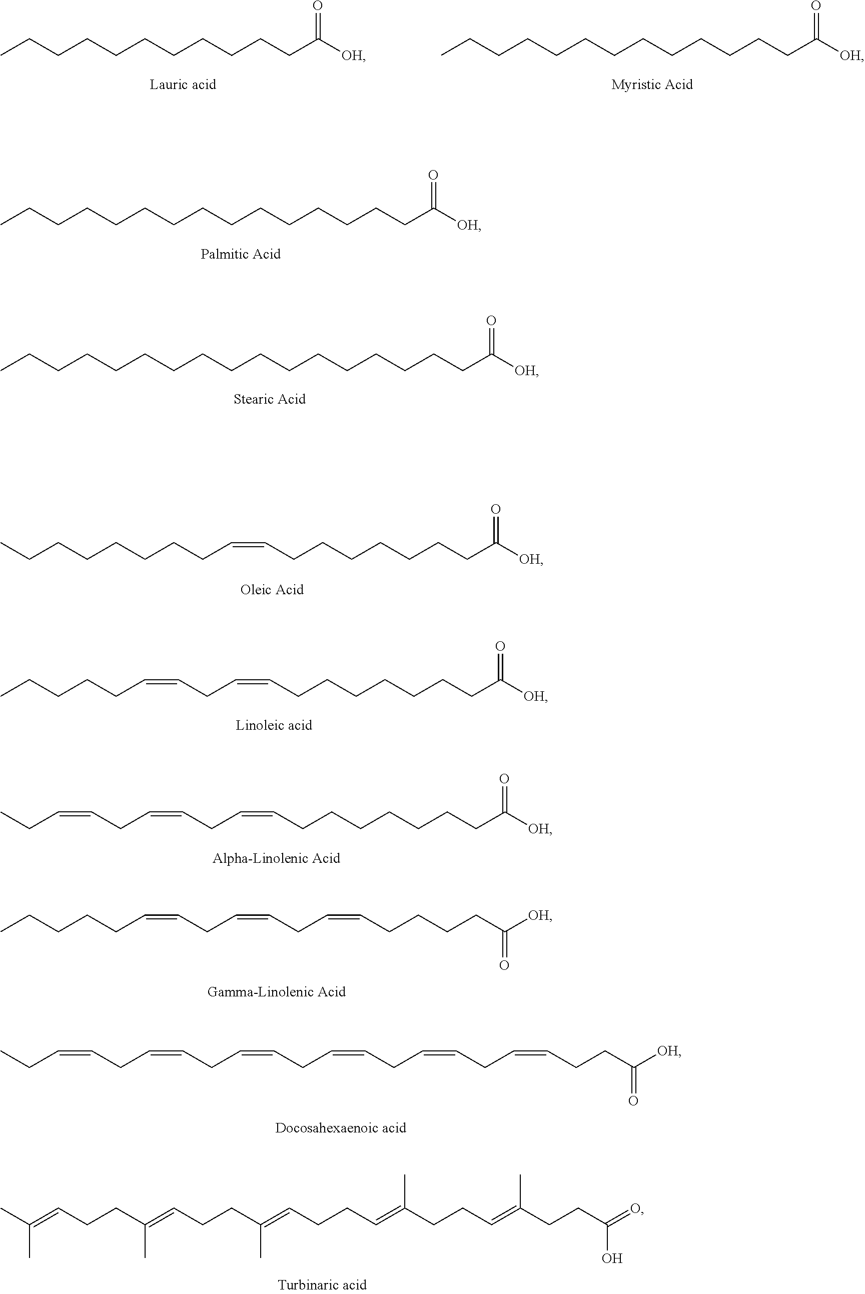

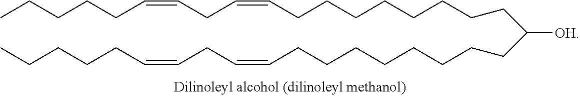 [SMILES: CCCCC/C=C\C/C=C\CCCCCCCCC(O)CCCCCCCC/C=C\C/C=C\CCCCC]

General Procedure for conjugation of lipids with C6-amino linked WV-942. A mixture of the lipid acid (55 μmol), HATU (50 μmol), Diisopropylethylamine (100 μmol) and NMP (500 μl) was shaken well at room temperature for 10 minutes, in a 3 ml plastic vial. This activated acid was pipetted into a plastic vial containing the CPG (5 μmol, CPG is linked to amino linked oligo). The contents of the vial was thoroughly mixed and shaken well for 12 hours. After this time the supernatant NMP was removed carefully. The CPG was washed with acetonitrile (1 ml×3) and dried in a speed vac. A 1:1 mixture (1 ml) of ammonium hydroxide and methyl amine (AMA) was added and heated at 35° C. for 1 hour with intermittent shaking. After 1 hour, the CPG was transferred into a small filtration cartridge, filtered, washed with DMSO (500 μl×2) and washed with water (1 ml×3). Filtrate and washings were combined and diluted to 10 ml using water. This solution was cooled to zero degrees celsius and neutralized with glacial acetic acid until pH of the solution reached 7.5. Crude product was analyzed by UV spectrometer, reverse phase HPLC and LC-MS. Purification of the crude product was done by RP HPLC.

TABLE 5
(Amount of CPG, Lipid acid, HATU, DIPEA
and NMP used for the coupling reactions).

| | | | | HATU | DIPEA | |
|---|---|---|---|---|---|---|
| | CPG | | | (50 μmol | (MW = 129) | |
| EXP | (5 | Acid | | (MW = | d = 0.726 | |
| # | μmol) | [55 μmol] | | 379.24) | (100 μmol) | NMP |
| 1 | 70.5 | Lauric acid | | 19 mg | 18 μL | 500 μL |
| | | (MW = 200.32) | | | | |
| | | 11.01 | mg | | | |
| 2 | 70.5 | Myristic Acid | | 19 mg | 18 μL | 500 μL |
| | | (MW = 228.38) | | | | |
| | | 12.56 | mg | | | |
| 3 | 70.5 | Palmitic acid | | 19 mg | 18 μL | 500 μL |
| | | (MW = 256.26) | | | | |
| | | 14.1 | mg | | | |
| 4 | 70.5 | Stearic acid | | 19 mg | 18 μL | 500 μL |
| | | (MW = 284.27) | | | | |
| | | 15.63 | mg | | | |
| 5 | 70.5 | Oleic acid | | 19 mg | 18 μL | 500 μL |
| | | (MW = 282.47) | | | | |
| | | 15.53 | g | | | |
| 6 | 70.5 | Linolenic acid | | 19 mg | 18 μL | 500 μL |
| | | (MW = 280.45) | | | | |
| | | 15.4 | mg | | | |
| 7 | 70.5 | α-Linoleic acid | | 19 mg | 18 μL | 500 μL |
| | | (MW = 278.44) | | | | |
| | | 15.3 | mg | | | |
| 8 | 70.5 | γ-Linoleic acid | | 19 mg | 18 μL | 500 μL |
| | | (MW = 278.44) | | | | |
| | | 15.3 | mg | | | |
| 9 | 70.5 | cis-DHA | | 19 mg | 18 μL | 500 μL |
| | | (MW = 328.24) | | | | |
| | | 18.05 | mg | | | |
| 10 | 70.5 | Turbinaric acid | | 19 mg | 18 μL | 500 μL |
| | | (MW = 400.36) | | | | |
| | | 22 | mg | | | |

After HPLC purification each fraction was analyzed by RP HPLC and LC-MS. Pure fractions were combined and solvent was removed under vacuum (speed vaac). Residue was dissolved in water and desalted (Triethyl ammonium ion was replaced with sodium ion) on a C-18 cartridge. Solvent was removed on a speed vaac and the residue was filtered through a centrifugal filter (Amicon Ultra-15 by Millipore), lyophilized and analyzed.

| Oligo | Conjugated | Total | Amount | Amount |
|---|---|---|---|---|
| (Wave#) | Acid | ODs | (μmol) | (mg) |
| WV2578 | Lauric Acid | 287 | 1.40 | 9.79 |
| WV2579 | Myristic Acid | 331 | 1.62 | 11.29 |
| WV2580 | Palmitic Acid | 268 | 1.31 | 9.14 |
| WV2581 | Stearic Acid | 265 | 1.30 | 9.04 |
| WV2582 | Oleic Acid | 262 | 1.28 | 8.94 |
| WV2583 | Linoleic Acid | 120 | 0.59 | 4.09 |
| WV2584 | α-Linolenic Acid | 285 | 1.39 | 9.72 |
| WV2585 | γ-Linolenic Acid | 297 | 1.45 | 10.13 |
| WV2586 | cis-DHA | 274 | 1.34 | 9.35 |
| WV2587 | Turbinaric acid | 186 | 0.91 | 6.35 |
| WV2588 | Dilinoleyl* | 345 | 1.69 | 11.77 |

*Synthesized on a solid support.

### Example 4. In Vitro Efficacy of Oligonucleotides Conjugated to Lipids

Cell Treatments and RNA Extraction

Primary human myoblasts from a patient with (deletion exon 48-50), DL 589.2 (deletion exon 51-55) were seeded into 12-well-plate pre-coated with matrigel (BD Biosciences) with density of 60×10³ cells per well in muscle cell proliferation medium (PromoCell GmbH, Heidelberg, Germany) at 37° C. with 500 CO₂. The next day, proliferation medium was replaced with muscle differentiation medium with 5% horse serum containing 10 μM oligos indicated in FIG. 1 and Table 1.

The oligonucleotide, which is identical to Drisapersen, has the sequence: 5′-mU*mC*mA*mA*mG*mG*mA*mA*mG*mA*mU*mG*mG*mC*mA*mU*mU*mU*mC*mU-3′, wherein * indicates a stereorandom phosphorothioate; and m indicates a 2′-OMe. WV-942 was conjugated with a lipid, as indicated in Table 1, on the 5′ end of the oligonucleotide.

TABLE 5
Lipids conjugated to biologically
active agent, oligonucleotide WV-942.

| Oligonucleotide | Conjugated Acid |
|---|---|
| WV-942 | — |
| WV-2578 | Lauric acid |
| WV-2579 | Myristic Acid |
| WV-2580 | Palmitic acid |
| WV-2581 | Stearic acid |
| WV-2582 | Oleic acid |
| WV-2583 | Linoleic acid |
| WV-2584 | Alpha-Linolenic acid |
| WV-2585 | Gamma-Linolenic acid |
| WV-2586 | cis-DHA |
| WV-2587 | Turbinaric acid |
| WV-2588 | Dilinoleyl |

Cells were differentiated for 4 days. Differentiation medium was then removed from each well and replaced with 500 μl of Trizol. Total RNA was extracted with 300 μl of phenol/chloroform, precipitated with 250 μl isopropanol, washed with 800 μl of 75% ethanol, and finally dissolved in 50 μl RNase free water.

Procedure of Nested PCR and Taqman Assay for DMD Skipping

Total cellular RNA was first reverse-transcribed into cDNA using the High-Capacity RNA-to-cDNA™ Kit from ThermoFisher Scientific following the protocol provided by the vendor.

For nested PCR, resulting cDNA was amplified sequentially using two sets of primers for nested PCR. PCR products were examined and visualized on agarose gels.

For the Taqman assay, skipped and unskipped transcripts in the cDNA were preamplified for 14 cycles using TaqMan® PreAmp Master Mix from ThermoFisher Scientific following the protocol provided. The amplification procedures are 95° C. for 10 min, then 14 cycles of 95° C. for 15 sec and 60° C. for 4 min. Preamplified cDNA was then analyzed for 40 cycles on LightCycler system (95° C. for 10 min, followed by 40 cycles of 95° C. for 15 sec and 60° C. for 1 min). Reactions contained 5 μl preamplified cDNA, 0.5 μl of skipped or unskipped Taqman assay and 0.5 μl Taqman assay for endogenous control, 4 μl water and 10 μl of Taman universal PCR master mix in a total volume of 20 μl. Data was analyzed using the LightCycler program to calculate Ct values. Endogenous controls include GAPDH, as well as muscle differentiation markers such as MyoD, desmin, myogenin, utrophin, myosin heavy chain and DMD itself.

Custom TaqMan MGB probes and primers were synthesized by Life Technologies using the following sequences.

Unskipped (exon 51)
Forward:

(SEQ ID NO: 2424)

GTGATGGTGGGTGACCTTGAG
Reverse:

(SEQ ID NO: 2425)

TTTGGGCAGCGGTAATGAG
Probe:

(SEQ ID NO: 2426)

CAAGCAGAAGGCAACAA
Skipped (exon 51)
Forward:

(SEQ ID NO: 2427)

TGAAAATAAGCTCAAGCAGACAAATC
Reverse:

(SEQ ID NO: 2428)

GACGCCTCTGTTCCAAATCC
Probe:

(SEQ ID NO: 2429)

CAGTGGATAAAGGCAACA

Results are shown in FIG. 1.

### Example 5. In Vivo Delivery of Compositions Comprising a Biologically Active Agent and a Lipid

In Vivo Oligo Treatment

Five weeks old mdx mice were dosed subcutaneously at 5 ml/kg at concentration of 10 mg/ml on Day 1. On Day 4, all animals were subjected to both terminal blood and tissue collection. Plasma was aliquoted into polypropylene tubes and stored at −70° C. For tissue collections, all animals were euthanized via CO₂ asphyxiation, and perfused using PBS. The following tissues were collected: liver, kidney, spleen, heart, thoracic diaphragm, gastrocnemius, quadriceps and triceps. Tissues were snap-frozen (in liquid nitrogen) and stored at −70° C.

Procedure:

In vivo biodistribution of the Control unconjugated ASO WV-942 or WV-942 conjugated to seven different lipids (WV-2588, -2581, -2582, -2584, -2585, -2586, -2587) was tested following a single subcutaneous administration to C57BL/10ScSn-Dmd^{mdx}/J male mice 5 weeks of age (Jackson Laboratory, Stock #001801). The study design is described in Table 1.

Animals were housed at 18° C. to 26° C. and 30% to 70% humidity two per cage in polycarbonate cages during acclimation and throughout the study. Housing included Beta Chip® and Enviro-Dri contact bedding. Standard chow and water were supplied ad libitum.

The study complied with all applicable sections of the Final Rules of the Animal Welfare Act regulations (Code of Federal Regulations, Title 9), the Public Health Service Policy on Humane Care and Use of Laboratory Animals from the Office of Laboratory Animal Welfare, and the Guide for the Care and Use of Laboratory Animals from the National Research Council. The protocol and any amendments or procedures involving the care or use of animals in this study were reviewed and approved by the Testing Facility Institutional Animal Care and Use Committee before the initiation of such procedures.

TABLE 6
Study design.

| | | | | Number | | | |
|---|---|---|---|---|---|---|---|
| | | | Dosing | of | Test Article | Dose | |
| | Test | Dose, | Route/Dosing | animals | Concentration, | Volume, | Termination |
| Group | Article | mg/kg | Day | (males) | mg/ml | ml/kg | Day |
| 1 | PBS | — | SC, Day 1 | 3 | 0 | 5 | Day 3 |
| 2 | WV-942 | 50 | SC, Day 1 | 3 | 10 | 5 | Day 3 |
| 3 | WV-2588 | 50 | SC, Day 1 | 3 | 10 | 5 | Day 3 |
| 4 | WV-2581 | 50 | SC, Day 1 | 3 | 10 | 5 | Day 3 |
| 5 | WV-2582 | 50 | SC, Day 1 | 3 | 10 | 5 | Day 3 |
| 6 | WV-2584 | 50 | SC, Day 1 | 3 | 10 | 5 | Day 3 |
| 7 | WV-2585 | 50 | SC, Day 1 | 3 | 10 | 5 | Day 3 |
| 8 | WV-2586 | 50 | SC, Day 1 | 3 | 10 | 5 | Day 3 |
| 9 | WV-2587 | 50 | SC, Day 1 | 3 | 10 | 5 | Day 3 |

Animals were euthanized via CO₂ asphyxiation 48 hours (±1 hour) after subcutaneous injection on Day 1. All animals were perfused using PBS. The following collected tissues (liver, kidney 2x, spleen, heart, thoracic diaphragm, gastroenemius, quadriceps, and triceps) were rinsed briefly with PBS, gently blotted dry, snap frozen (liquid N2) in polypropylene tubes and storaged at −70° C. until processing for further analysis.

Oligo Quantification

Briefly, each mouse tissue was weighted and lysed in tissue lysis buffer.

Hybridization Assay to Detect ASO: Sandwich

Methods:

Probe:

Capture probe: /5AmMC12/A+GA+AA+TG+CC+A (SEQ ID NO: 2430)

Detection probe: T+CT+TC+CT+TG+A/3Bio/ (SEQ ID NO: 2431)

Plate:

Coat Pierce® Amine-binding, Maleic Anhydride 96-Well Plates, with diluted Capture probe at 500 nM in 2.5% NaHCO₃, at 37 C for at least 1 hour (or 4 C overnight). After wash with PBST (1×PBS+0.1% Tween-20), block in 5% fat-free milk/PBST at 37 C for >1 hour.

Tissue Sample Preparation

Weight tissue pieces, add 4 volume of lysis buffer to tissue to achieve 0.2 g tissue/ml, in tissue lysis buffer (IGEPAL 0.5%, 100 mM NaCl, 5 mM EDTA, 10 mM Tris pH8, protease K 300 ug/ml). The homogenate was generated by Bullet Blender (NextAdvance).

Standard Curve:

Dilute Test Article into non-treated blank tissue homogenates (matrix) at 10-50 ug/ml (50-250 ug/g tissue). The standard was further serial diluted 1:1 with matrix for 8 points to form standard curve series.

Hybrid-ELISA:

Dilute Standard Curve samples, treated tissue homogenates 100-500 times with hybridization buffer (4 M Guanidine; 0.33% N-Lauryl Sarcosine; 25 mM Sodium Citrate; 10 mM DTT). 20 ul of diluted tissue samples were mixed with 180 ul of detection probe diluted in PBST at 333 nM. Samples were denatured using following condition: 65 C, 10 min; 95 C, 15 min; 4 C, ∞. Add 50 ul/well denatured samples into coated 96 wells. Incubate at 4 C for overnight. Wash plate 3 times with PBST. Add 1:2000 dilution of streptavidin-AP in PBST. Incubate at room temperature for 1 hour. Wash plate 5 times×2 cycles with PBST on Molecular Device plate wash machine. Add 100 ul/well AttoPhos substrates. Incubate for 10 min, read plate at Molecular Device M5 in fluorescence channel: Ex435 nm, Em555 nm. Take another read at 20 min. The ASO concentration is calculated against Standard Curve by using either linear curve fit or 4-parameter curve fit.

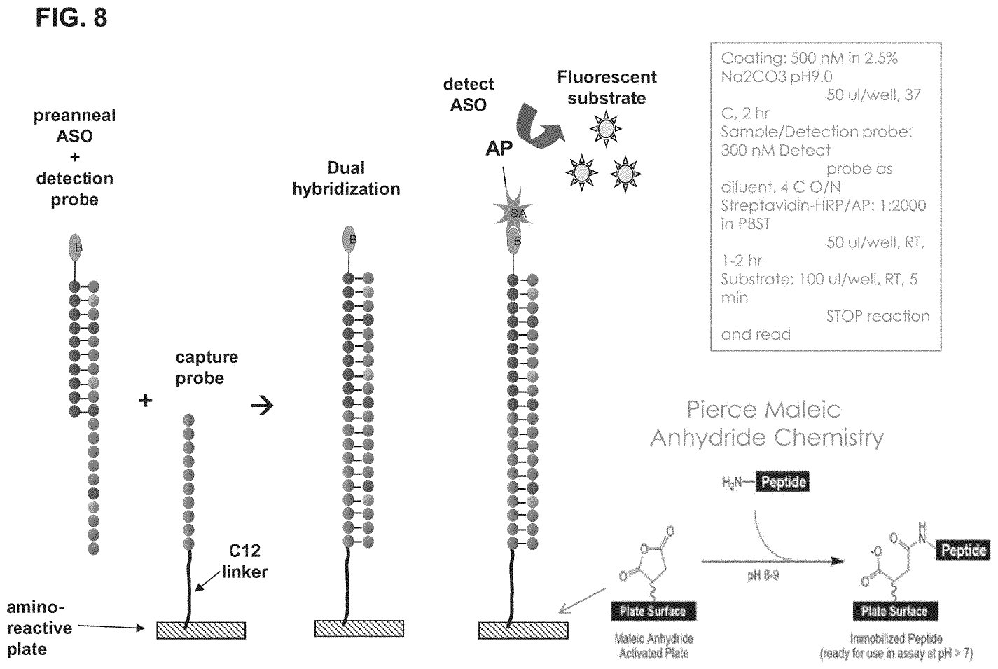

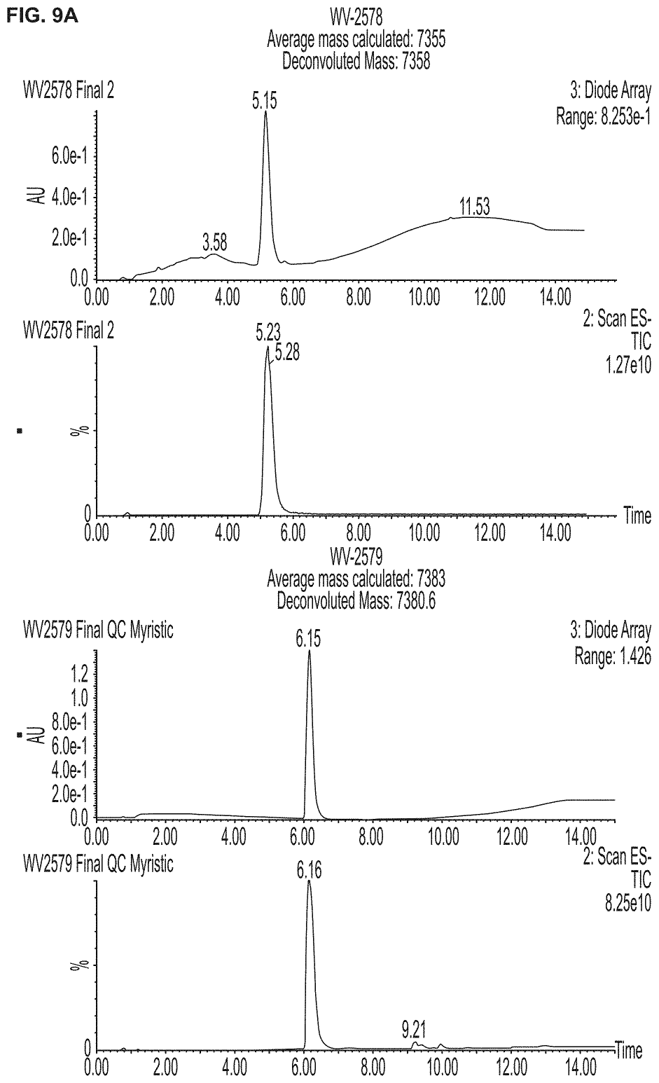

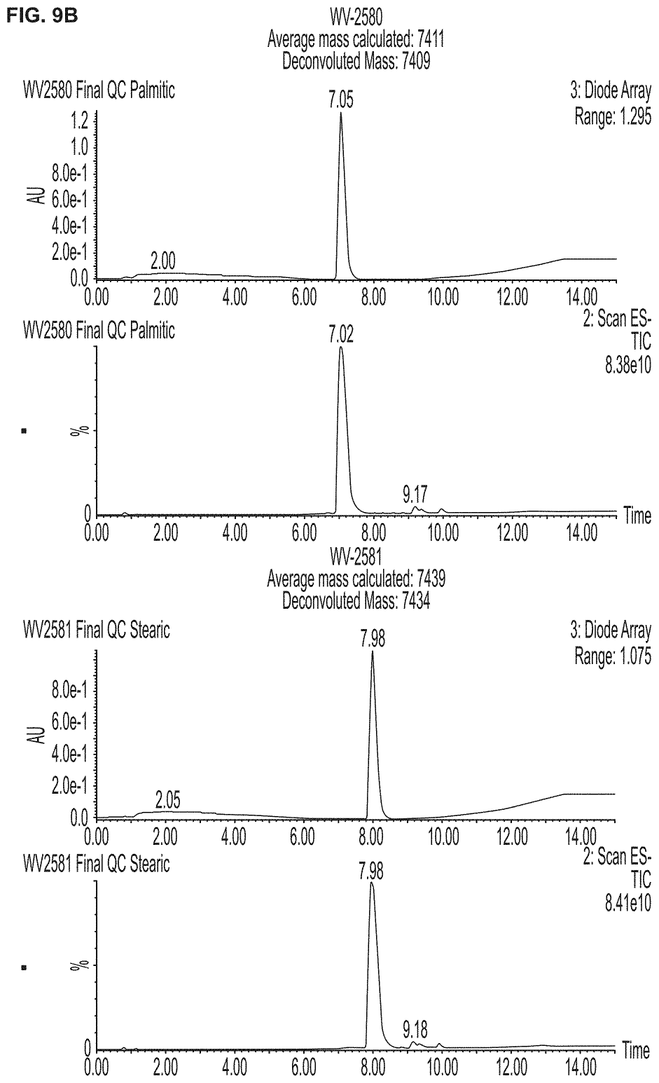

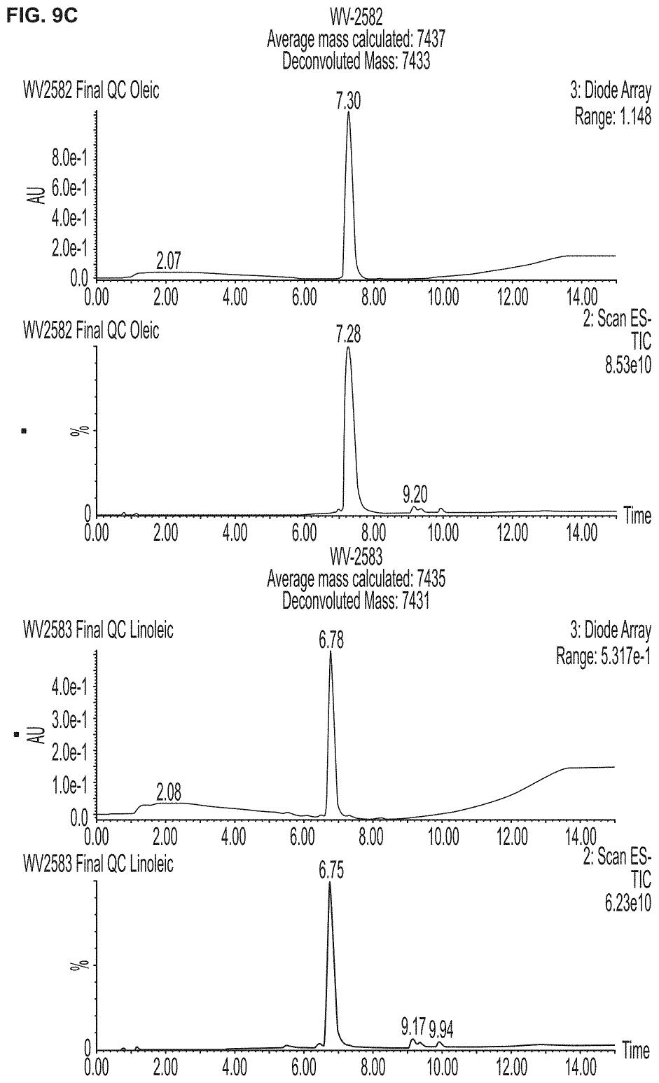

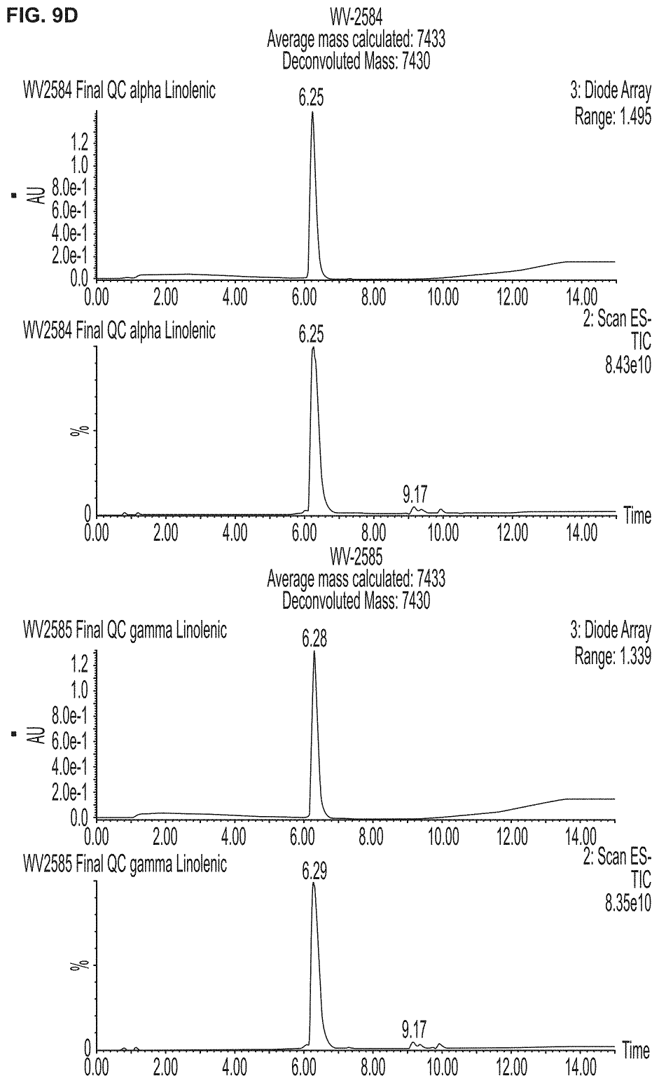

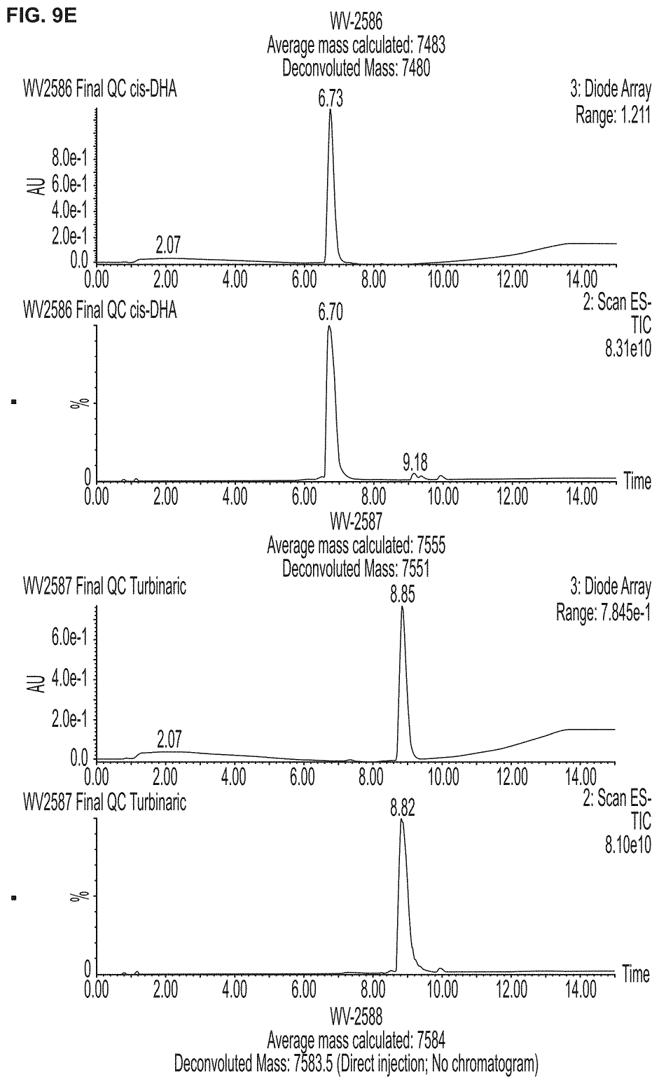

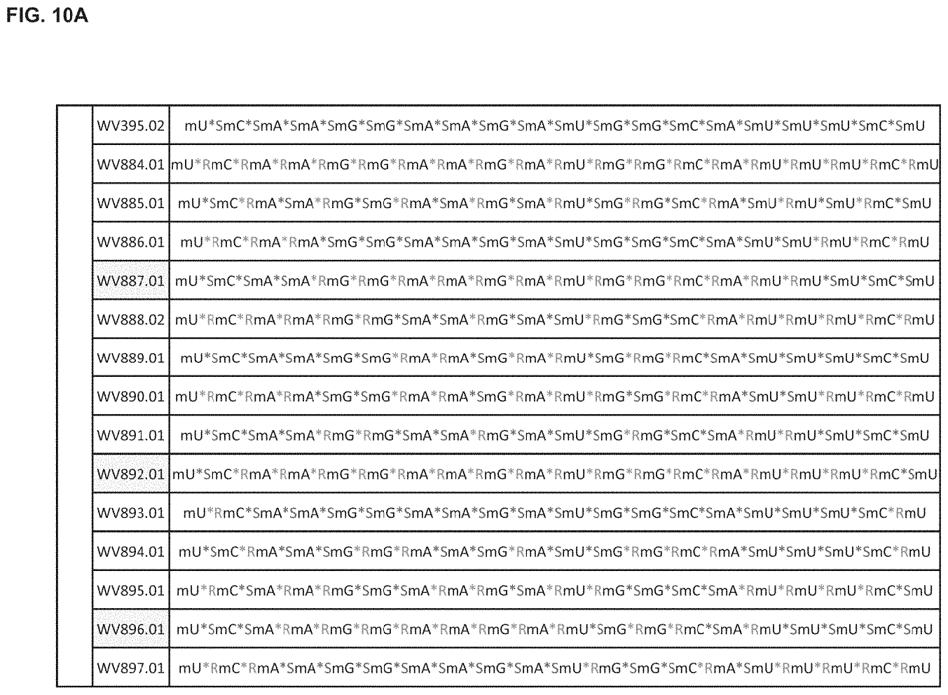

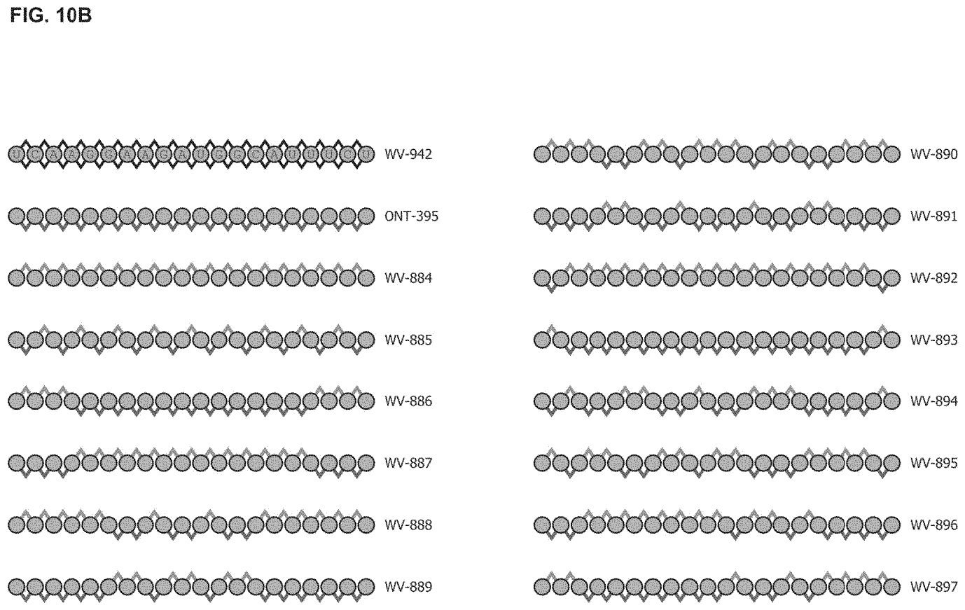

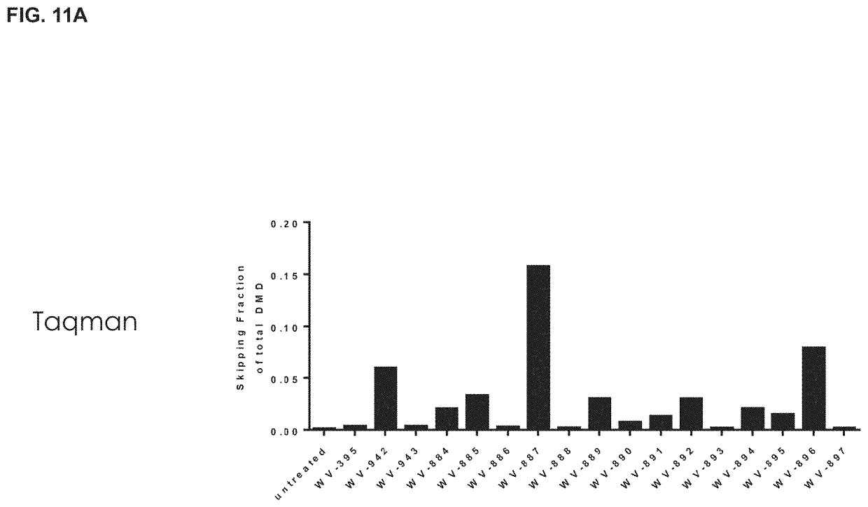

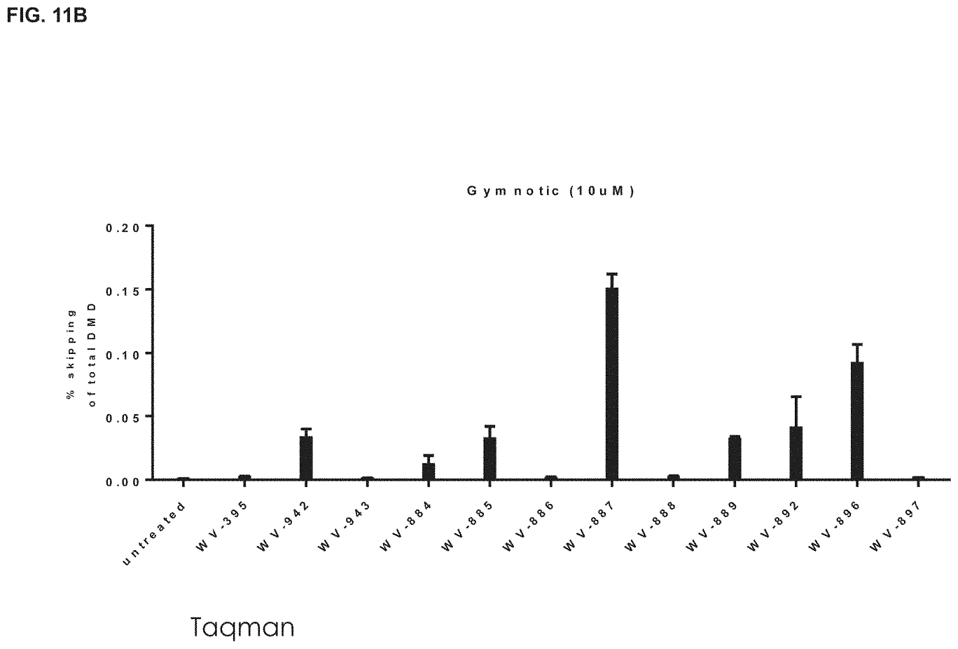

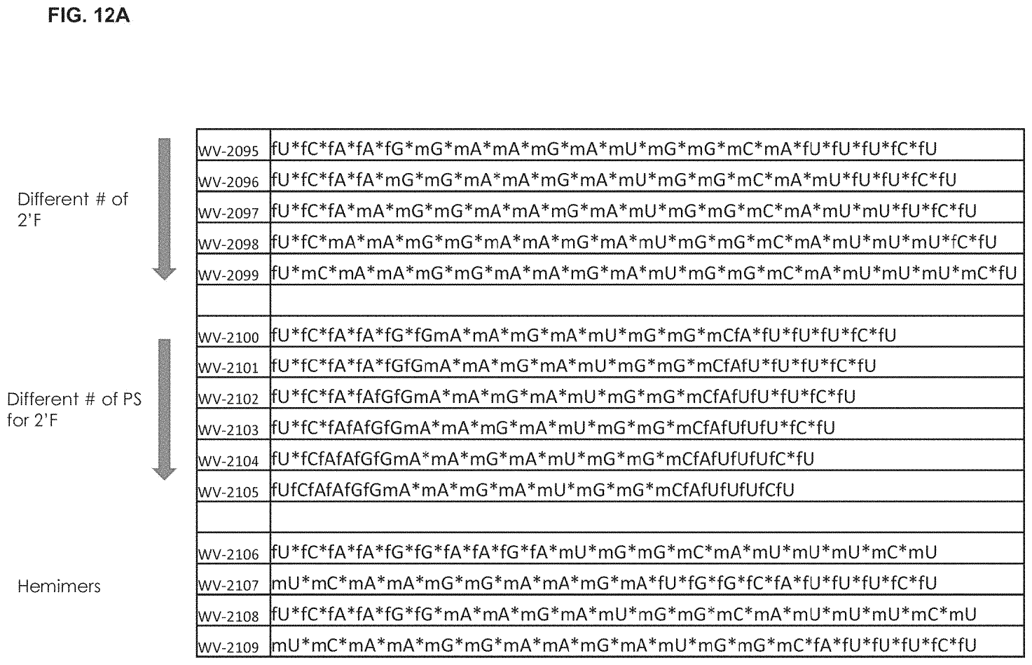

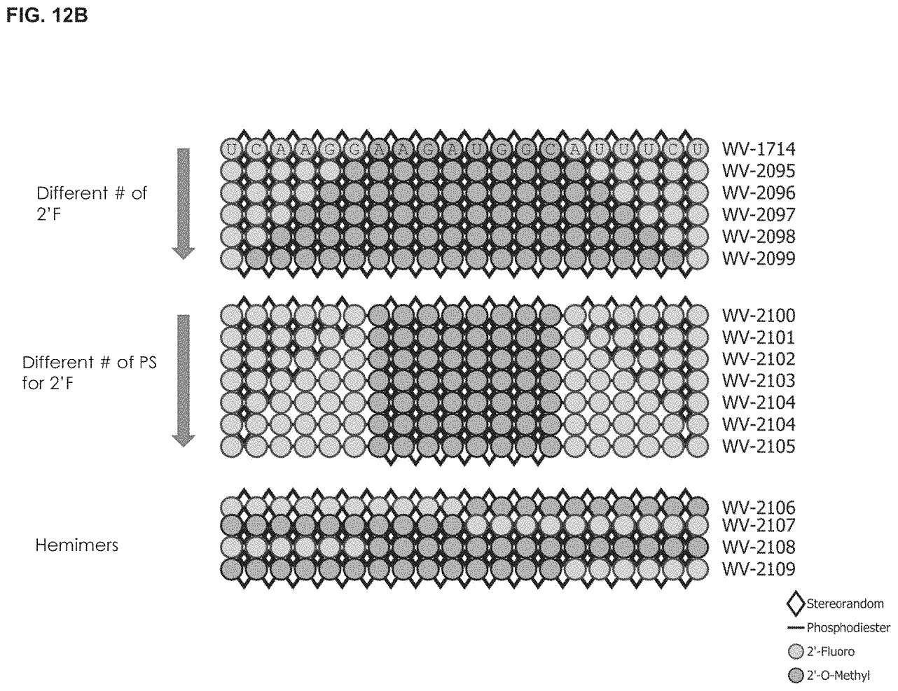

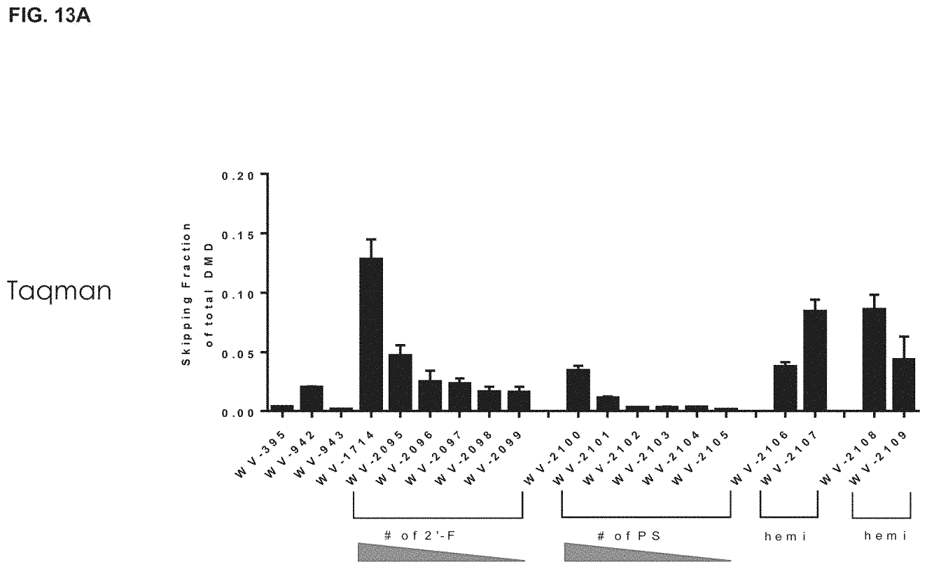

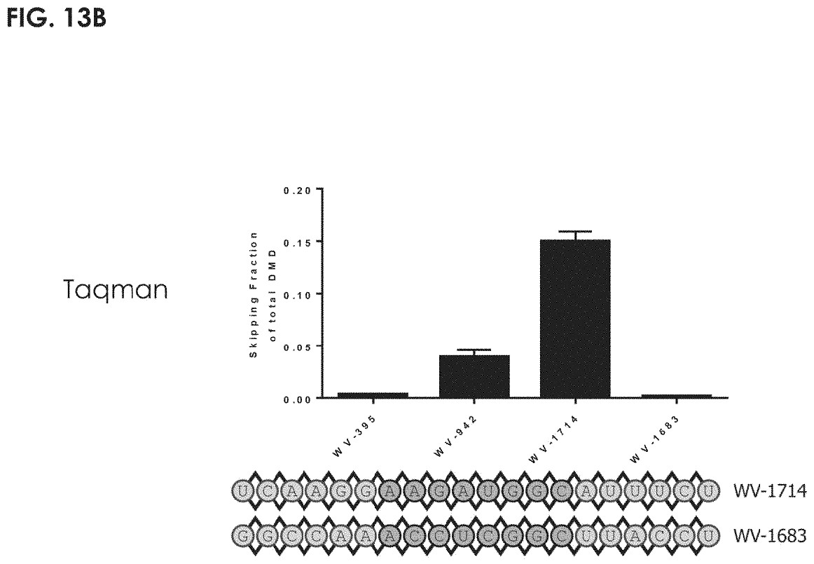

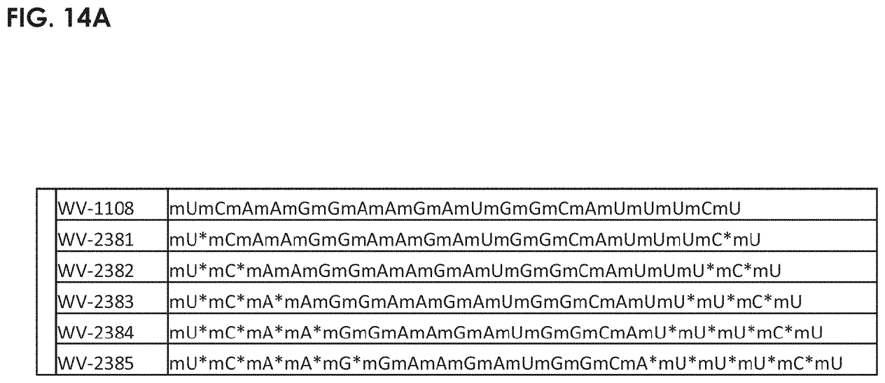

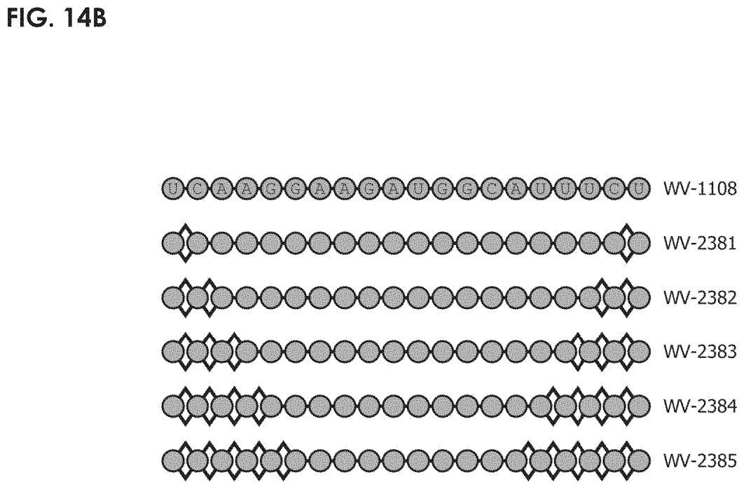

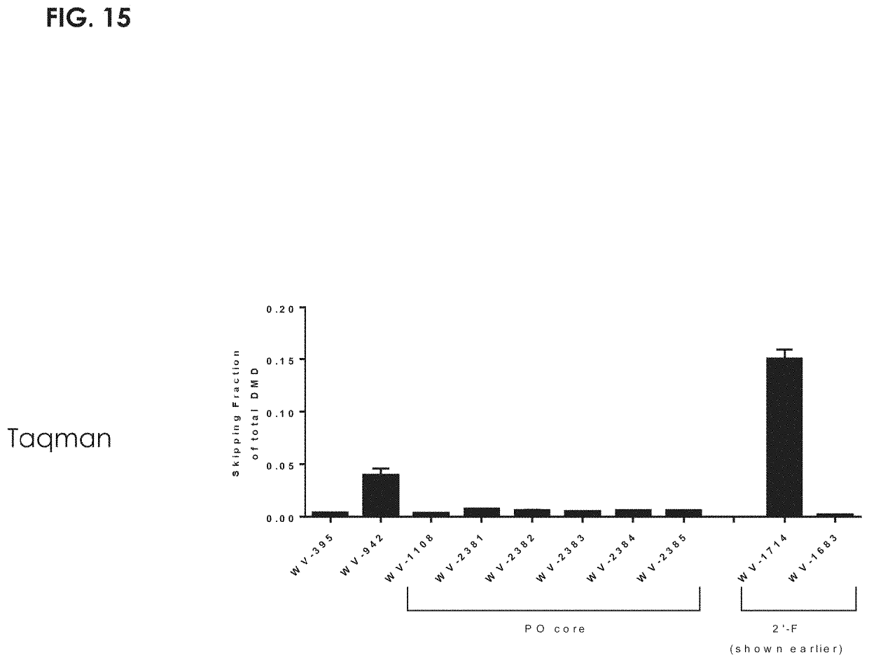

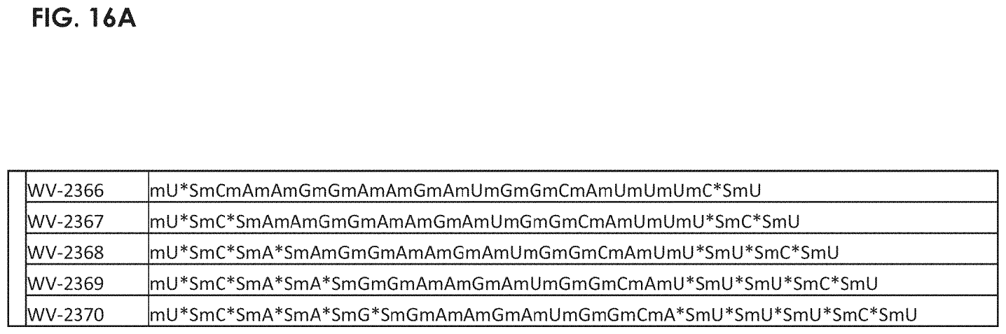

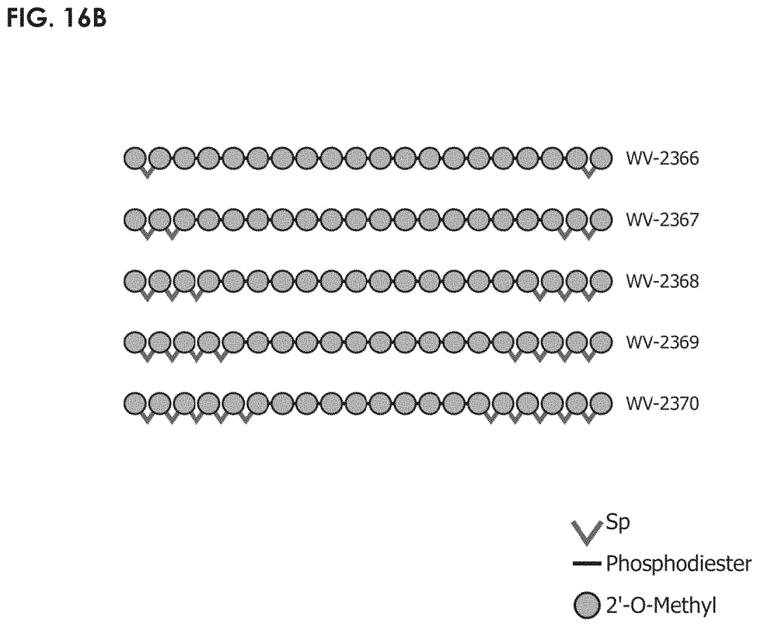

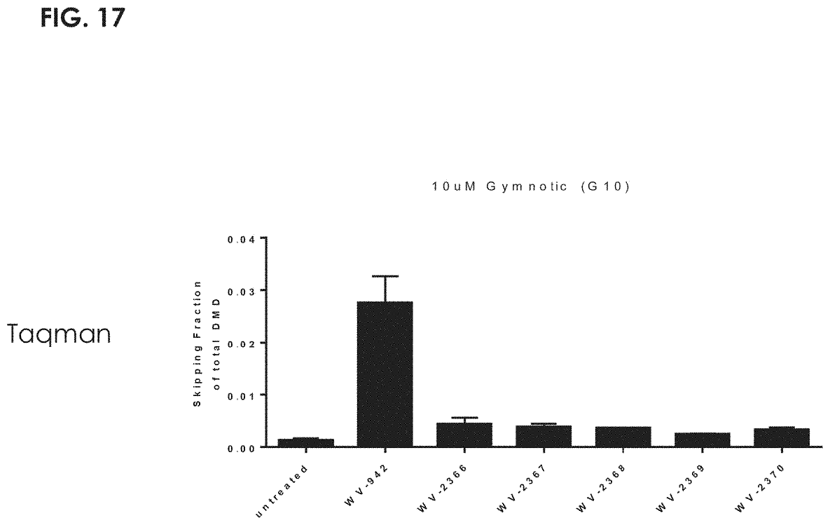

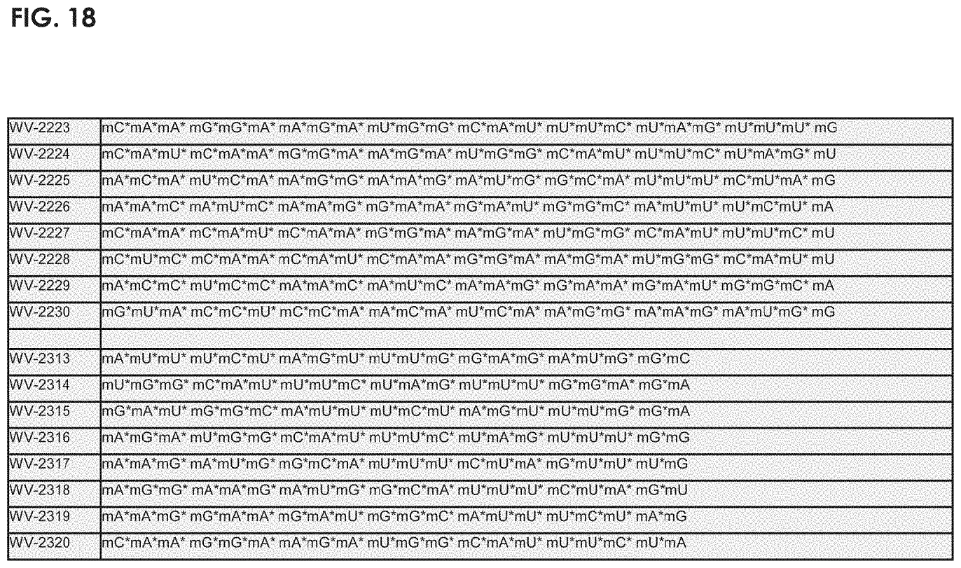

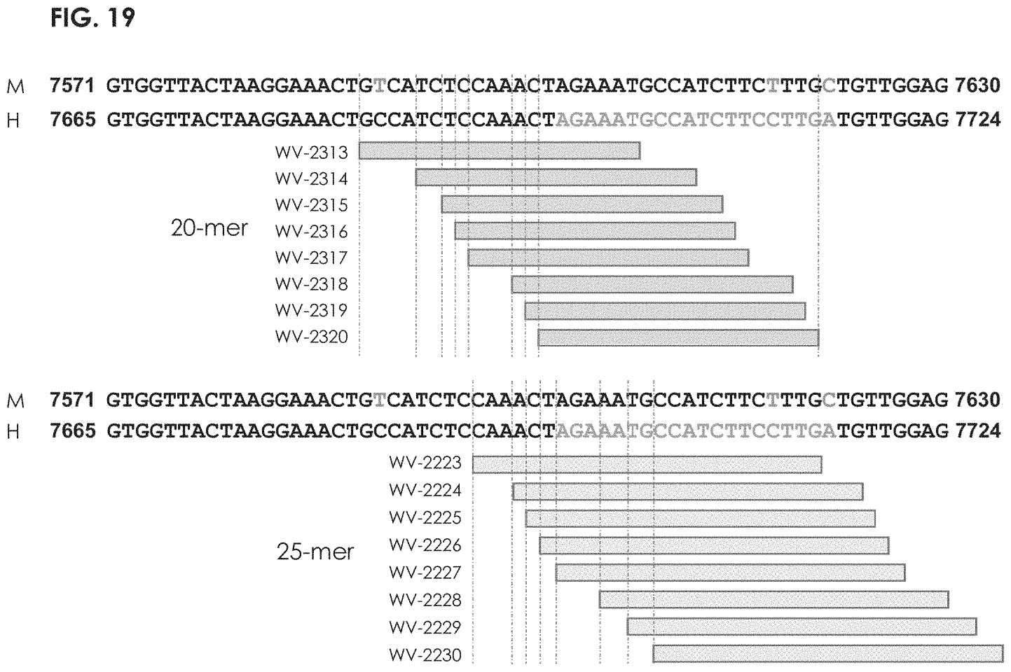

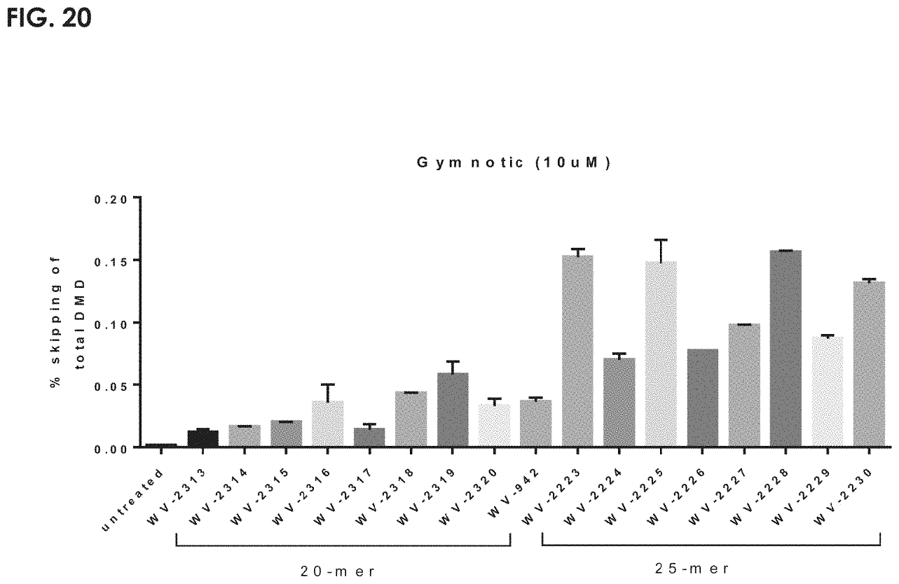

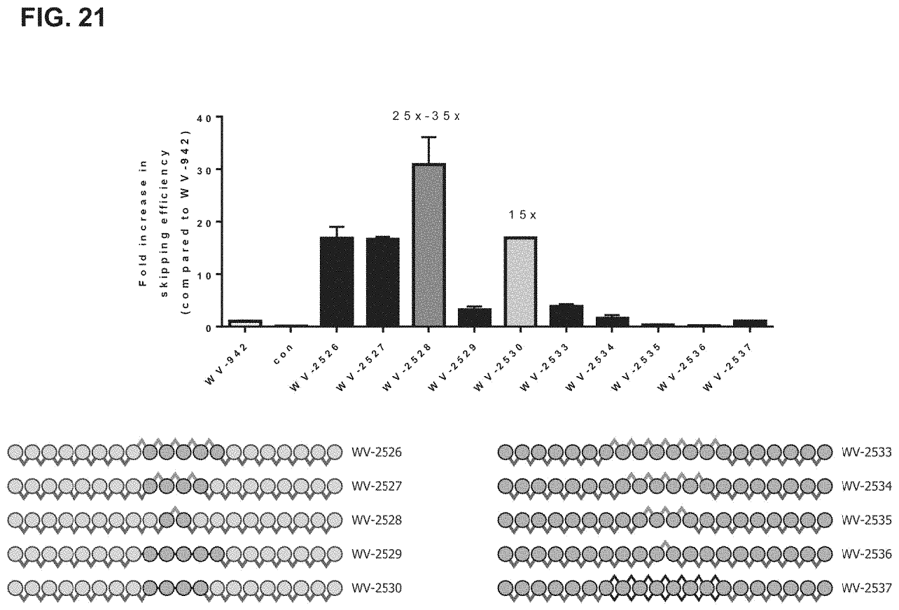

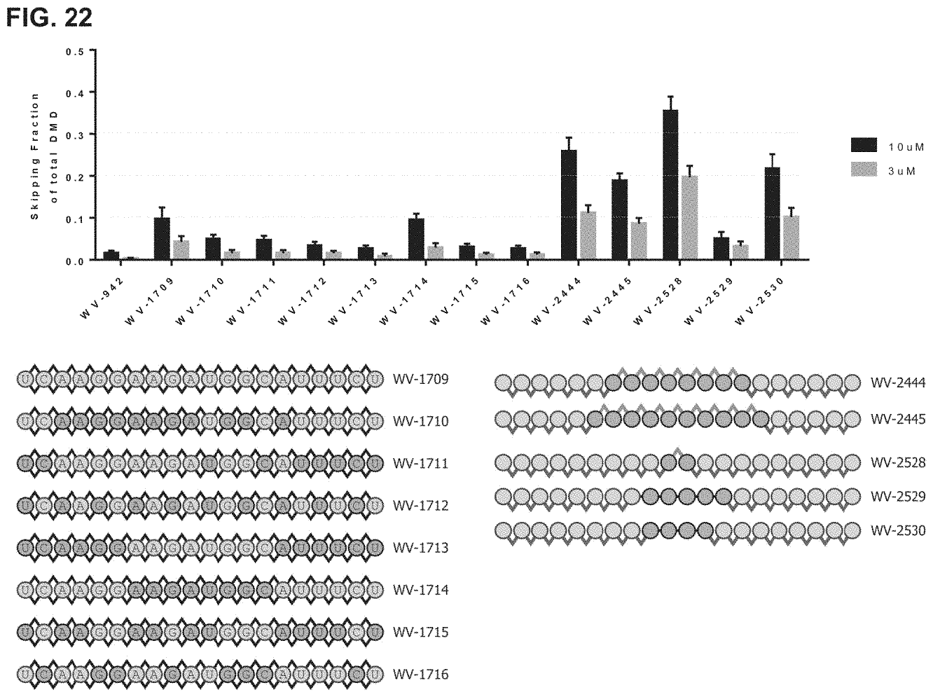

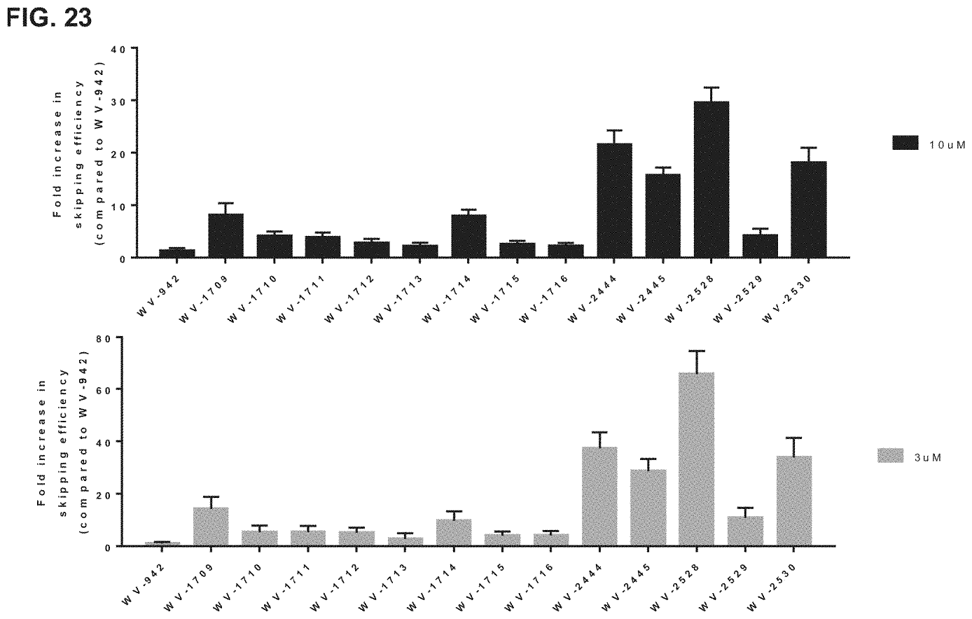

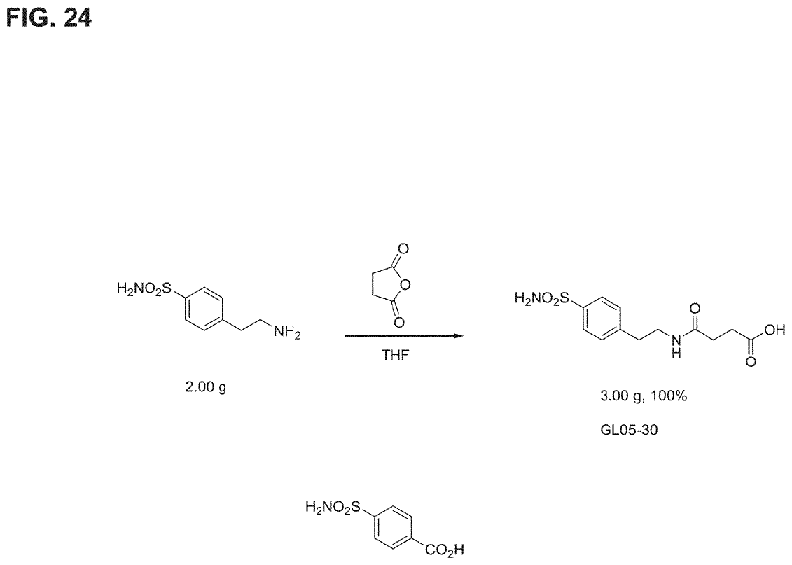

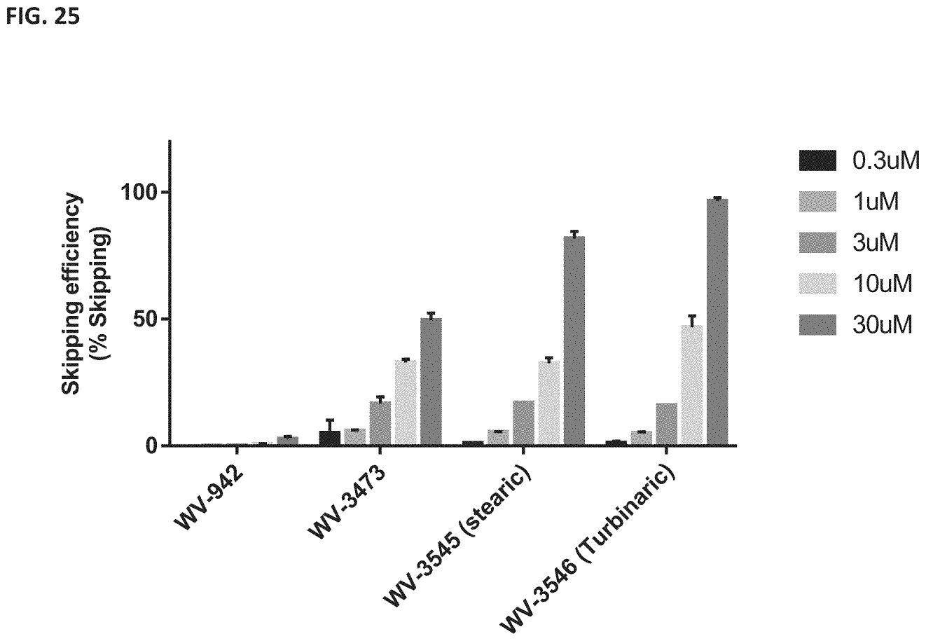

An example protocol is illustrated in FIG. 8.

### Example 6. Example Assay for Measuring TLR9 Agonist and Antagonist Activities

Various assays can be utilized to assay TLR9 activities of provided compositions in accordance with the present disclosure. In one example human TLR9 report assay, HEK-Blue™ TLR9 cells which stably overexpress the human TLR9 gene and an NF-kB inducible secreted embryonic alkaline phosphatase (SEAP) were obtained from Invivogen (San Diego, CA, USA). Oligonucleotides at indicated concentrations were plated into 96-well-plates in the final volume of 20 mL in water. 4×10⁴ HEK-Blue TLR9 cells were added to each well in a volume of 180 mL in SEAP detection medium. In certain experiments, oligonucleotides were added in the presence or absence of various concentrations of TLR9 agonists (e.g., oligonucleotide ODN2006), and the cultures were continued for 16 h. At the end of the treatment, OD was measured at 655 nM. The results are expressed as fold change in NF-κB activation over phosphate buffered saline (PBS)-treated cells.

### Example 7. Example In Vivo Delivery of Provided Compounds and Compositions

Example in vivo oligonucleotide treatment: Five-week-old mdx mice were dosed i.v. or subcutaneously at 5 mL/kg at concentration of 10 mg/mL on Day 1. On Day 4 (or other days as desired), all animals were subjected to both terminal blood and tissue collection. Plasma was aliquoted into polypropylene tubes and stored at −70° C. For tissue collections, all animals were euthanized via CO₂ asphyxiation, and perfused using PBS. The following tissues were also collected: liver, kidney, spleen, heart, thoracic diaphragm, gastrocnemius, quadriceps and triceps. Tissues were snap-frozen (in liquid nitrogen) and stored at −70° C.

Example procedure: In vivo biodistribution of the control oligonucleotide WV-942 and oligonucleotides to be tested (e.g., WV-2588, WV-2581, WV-2582, WV-2584, WV-2585, WV-2586, WV-2587, etc.) was tested following a single subcutaneous administration to C57BL/10ScSn-Dmd^{mdx}/J male mice 5 weeks of age (Jackson Laboratory, Stock #001801). Animals were housed at 18° C. to 26° C. and 30% to 70% humidity two per cage in polycarbonate cages during acclimation and throughout the study. Housing included Beta Chip® and Enviro-Dri contact bedding. Standard chow and water were supplied ad libitum. The study complied with all applicable sections of the Final Rules of the Animal Welfare Act regulations (Code of Federal Regulations, Title 9), the Public Health Service Policy on Humane Care and Use of Laboratory Animals from the Office of Laboratory Animal Welfare, and the Guide for the Care and Use of Laboratory Animals from the National Research Council. The protocol and any amendments or procedures involving the care or use of animals in this study were reviewed and approved by the Testing Facility Institutional Animal Care and Use Committee before the initiation of such procedures.

Animals were euthanized via CO₂ asphyxiation 48 hours (+1 hour) after subcutaneous injection on Day 1. All animals were perfused using PBS. The following collected tissues (liver, kidney 2x, spleen, heart, thoracic diaphragm, gastrocnemius, quadriceps, and triceps) were rinsed briefly with PBS, gently blotted dry, snap frozen (liquid N2) in polypropylene tubes and stored at −70° C. until processing for further analysis.

Oligonucleotide quantification: Briefly, each mouse tissue was weighted and lysed in tissue lysis buffer.

Hybridization assay to detect ASO: Sandwich

Methods:

Probe: Capture probe: /5AmMC12/A+GA+AA+TG+CC+A (SEQ ID NO: 2430); Detection probe: T+CT+TC+CT+TG+A/3Bio/ (SEQ ID NO: 2431)

Plate: Coat Pierce® Amine-binding, Maleic Anhydride 96-Well Plates, with diluted Capture probe at 500 nM in 2.5% NaHCO₃, at 37° C. for at least 1 hour (or 4° C. overnight). After wash with PBST (1×PBS+0.1% Tween-20), block in 5% fat-free milk/PBST at 37° C. for >1 hour.

Tissue sample preparation: Weigh tissue pieces, add 4 volumes of lysis buffer to tissue to achieve 0.2 g tissue/mL, in tissue lysis buffer (IGEPAL 0.5%, 100 mM NaCl, 5 mM EDTA, 10 mM Tris pH8, protease K 300 μg/mL). The homogenate was generated by Bullet Blender (NextAdvance).

Standard Curve: Dilute Test Article into non-treated blank tissue homogenates (matrix) at 10-50 μg/mL (50-250 μg/g tissue). The standard was further serial diluted 1:1 with matrix for 8 points to form standard curve series.

Hybrid-ELISA: Dilute Standard Curve samples, treated tissue homogenates 100-500 times with hybridization buffer (4 M Guanidine; 0.33% N-Lauryl Sarcosine; 25 mM Sodium Citrate; 10 mM DTT). 20 μL of diluted tissue samples were mixed with 180 μL of detection probe diluted in PBST at 333 nM. Samples were denatured using following condition: 65° C., 10 min; 95° C., 15 min; 4° C., ∞. Add 50 μL/well denatured samples into coated 96 wells. Incubate at 4° C. for overnight. Wash plate 3 times with PBST. Add 1:2000 dilution of streptavidin-AP in PBST. Incubate at room temperature for 1 hour. Wash plate 5 times×2 cycles with PBST on Molecular Device plate wash machine. Add 100 μL/well AttoPhos substrates. Incubate for 10 min, read plate at Molecular Device M5 in fluorescence channel: Ex435 nm, Em555 nm. Take another read at 20 min. Oligonucleotide concentration is calculated against Standard Curve by using either linear curve fit or 4-parameter curve fit.

Example test results were presented in the Figures, e.g., 31A-31D, demonstrating that provided oligonucleotides comprising lipid moieties have improved properties (e.g., distribution, metabolism, etc.).

### Example 8. Example synthesis of 1,7,14-trioxo-12,12-bis((3-oxo-3-((3-(4-sulfamoylbenzamido)propyl)amino)propoxy)methyl)-1-(4-sulfamoylphenyl)-10-oxa-2,6,13-triazaoctadecan-18-oic acid

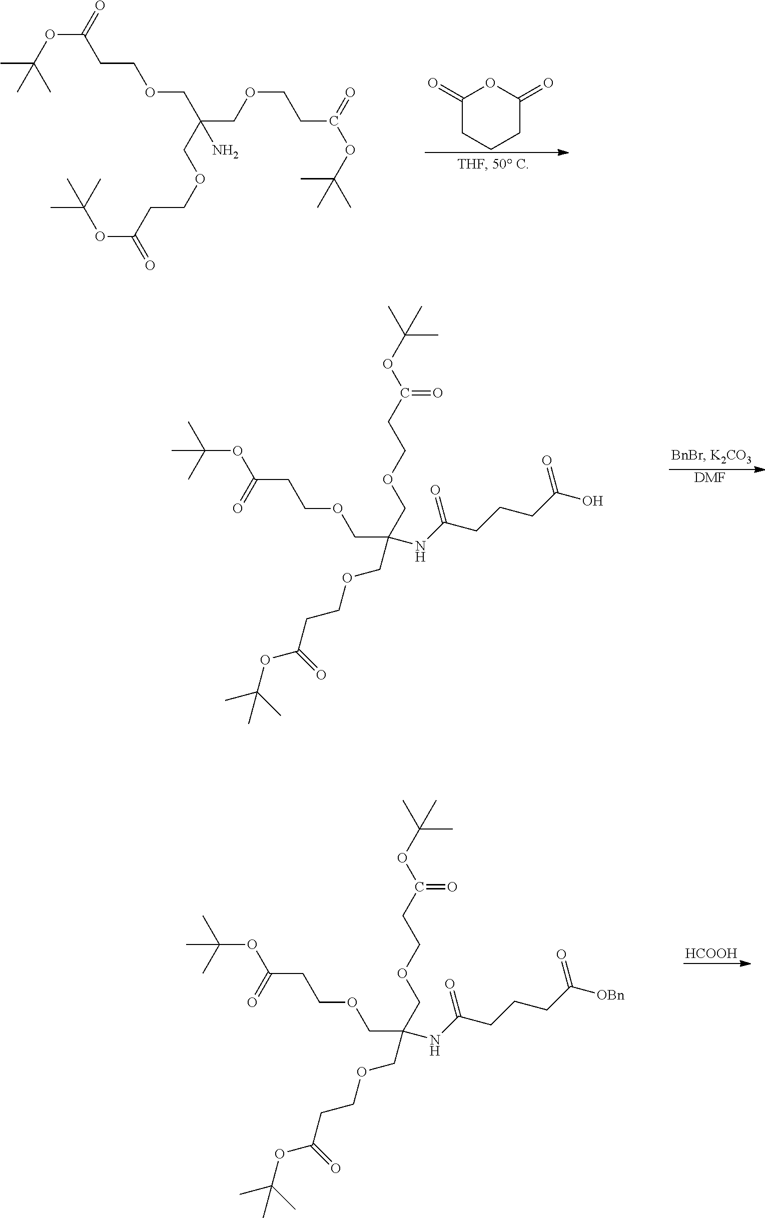

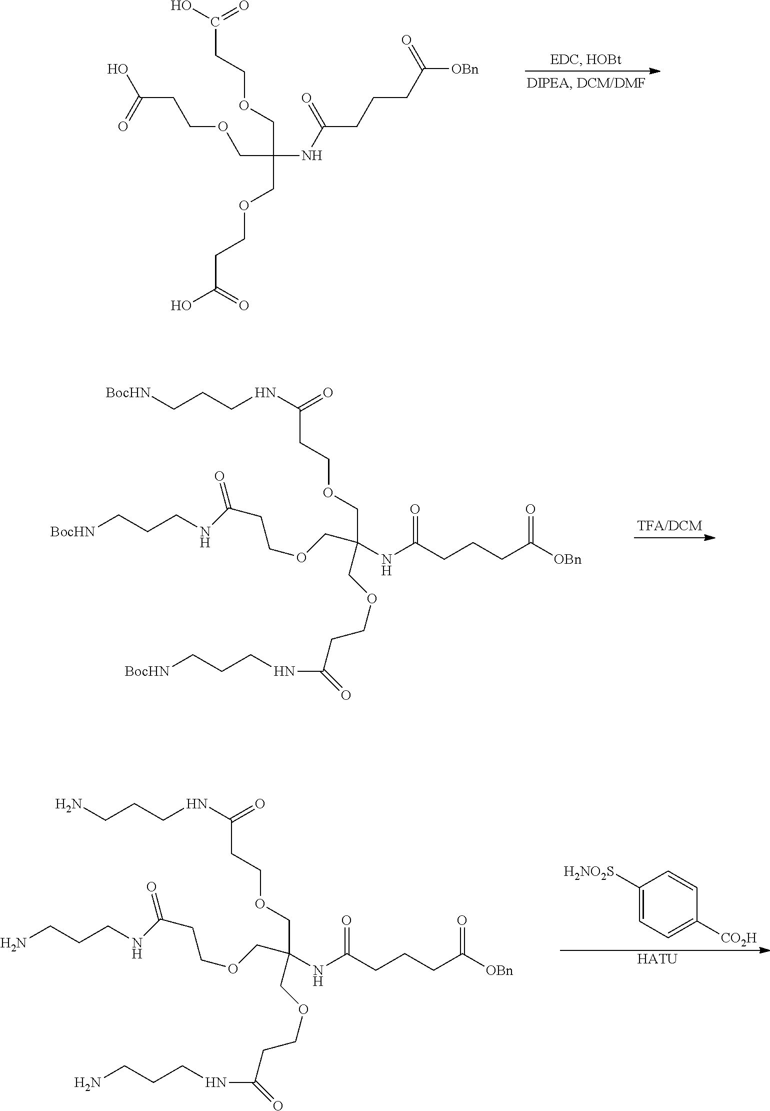

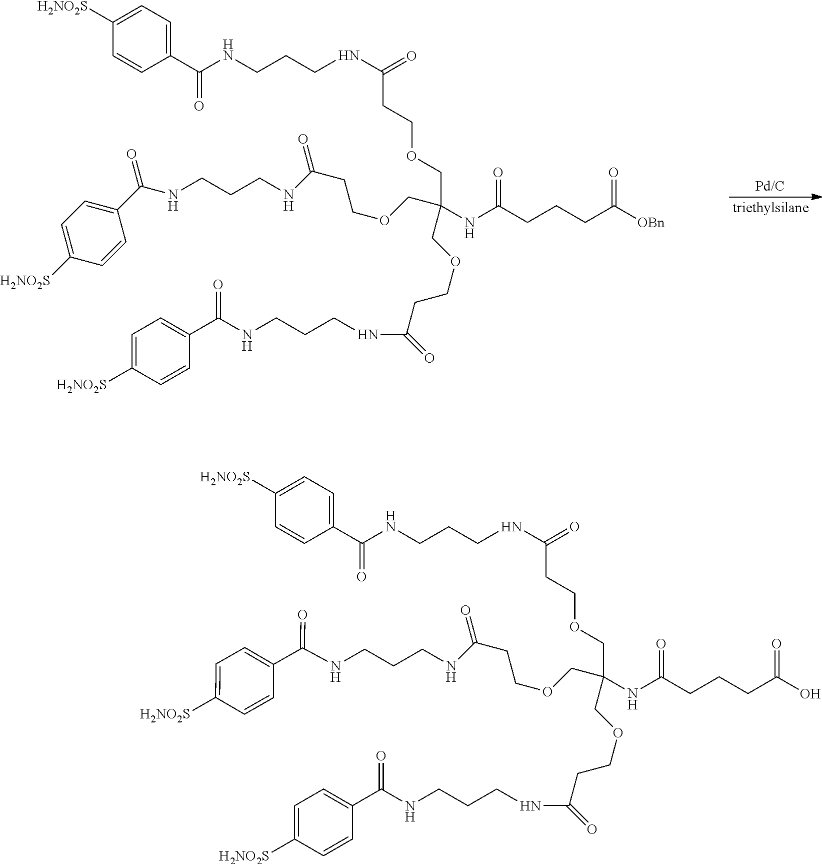 [SMILES: NS(=O)(=O)c1ccc(C(=O)NCCCNC(=O)CCOCC(COCCC(=O)NCCCNC(=O)c2ccc(S(N)(=O)=O)cc2)(COCCC(=O)NCCCNC(=O)c2ccc(S(N)(=O)=O)cc2)NC(=O)CCCC(=O)OCc2ccccc2)cc1 ; NS(=O)(=O)c1ccc(C(=O)NCCCNC(=O)CCOCC(COCCC(=O)NCCCNC(=O)c2ccc(S(N)(=O)=O)cc2)(COCCC(=O)NCCCNC(=O)c2ccc(S(N)(=O)=O)cc2)NC(=O)CCCC(=O)O)cc1]

Step 1: A solution of di-tert-butyl 3,3′-((2-amino-2-((3-(tert-butoxy)-3-oxopropoxy)methyl)propane-1,3-diyl)bis(oxy))dipropanoate (4.0 g, 7.91 mmol) and dihydro-2H-pyran-2,6(3H)-dione (0.903 g, 7.91 mmol) in THF (40 mL) was stirred at 50° C. for 3 hrs and at rt for 3 hrs. LC-MS showed desired product. Solvent was evaporated to give 5-((9-((3-(tert-butoxy)-3-oxopropoxy)methyl)-2,2,16,16-tetramethyl-4,14-dioxo-3,7,11,15-tetraoxaheptadecan-9-yl)amino)-5-oxopentanoic acid, which was directly used for next step without purification.

Step 2: To a solution of 5-((9-((3-(tert-butoxy)-3-oxopropoxy)methyl)-2,2,16,16-tetramethyl-4,14-dioxo-3,7,11,15-tetraoxaheptadecan-9-yl)amino)-5-oxopentanoic acid (4.90 g, 7.91 mmol) and (bromomethyl)benzene (1.623 g, 9.49 mmol) in DMF was added anhydrous K₂CO₃ (3.27 g, 23.73 mmol). The mixture was stirred at 40° C. for 4 hrs and at room temperature for overnight. Solvent was evaporated under reduced pressure. The reaction mixture was diluted with EtOAc, washed with water, dried over anhydrous sodium sulfate, and concentrated under reduced pressure to give a residue, which was purified by ISCO eluting with 10% EtOAc in hexane to 50% EtOAc in hexane to give di-tert-butyl 3,3′-((2-(5-(benzyloxy)-5-oxopentanamido)-2-((3-(tert-butoxy)-3-oxopropoxy)methyl)propane-1,3-diyl)bis(oxy))dipropanoate (5.43 g, 7.65 mmol, 97% yield) as a colorless oil. ¹H NMR (400 MHz, Chloroform-d) δ 7.41-7.28 (m, 5H), 6.10 (s, 1H), 5.12 (s, 2H), 3.72-3.60 (m, 12H), 2.50-2.38 (m, 8H), 2.22 (t, J=7.3 Hz, 2H), 1.95 (p, J=7.4 Hz, 2H), 1.45 (s, 27H); MS (ESI), 710.5 (M+H)+.

Step 3: A solution of di-tert-butyl 3,3′-((2-(5-(benzyloxy)-5-oxopentanamido)-2-((3-(tert-butoxy)-3-oxopropoxy)methyl)propane-1,3-diyl)bis(oxy))dipropanoate (5.43 g, 7.65 mmol) in formic acid (50 mL) was stirred at room temperature for 48 hrs. LC-MS showed the reaction was not complete. Solvent was evaporated under reduced pressure. The crude product was re-dissolved in formic acid (50 mL) and was stirred at room temperature for 6 hrs. LC-MS showed the reaction was complete. Solvent was evaporated under reduced pressure, co-evaporated with toluene (3×) under reduced pressure, and dried under vacuum to give 3,3′-((2-(5-(benzyloxy)-5-oxopentanamido)-2-((2-carboxyethoxy)methyl)propane-1,3-diyl)bis(oxy))dipropanoic acid (4.22 g, 7.79 mmol, 100% yield) as a white solid. ¹H NMR (500 MHz, DMSO-d₆) δ 12.11 (s, 3H), 7.41-7.27 (m, 5H), 6.97 (s, 1H), 5.07 (s, 2H), 3.55 (d, J=6.4 Hz, 6H), 2.40 (t, J=6.3 Hz, 6H), 2.37-2.26 (m, 2H), 2.08 (t, J=7.3 Hz, 2H), 1.70 (p, J=7.4 Hz, 2H); MS (ESI), 542.3 (M+H)⁺.

Step 4: To a solution of 3,3′-((2-(5-(benzyloxy)-5-oxopentanamido)-2-((2-carboxyethoxy)methyl)propane-1,3-diyl)bis(oxy))dipropanoic acid (4.10 g, 7.57 mmol) and HOBt (4.60 g, 34.1 mmol) in DCM (60 mL) and DMF (15 mL) at 0° C. was added tert-butyl (3-aminopropyl)carbamate (5.94 g, 34.1 mmol), EDAC HCl salt (6.53 g, 34.1 mmol) and DIPEA (10.55 mL, 60.6 mmol). The reaction mixture was stirred at 0° C. for 15 minutes and at room temperature for 20 hrs. LC-MS showed the reaction was not complete. EDAC HCl salt (2.0 g) and tert-butyl (3-aminopropyl)carbamate (1.0 g) was added into the reaction mixture. The reaction mixture was stirred at room temperature for 4 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate 5 (6.99 g, 6.92 mmol, 91% yield) as a white solid. ¹H NMR (500 MHz, Chloroform-d) δ 7.35 (t, J=4.7 Hz, 5H), 6.89 (s, 3H), 6.44 (s, 1H), 5.22 (d, J=6.6 Hz, 3H), 5.12 (s, 2H), 3.71-3.62 (m, 12H), 3.29 (q, J=6.2 Hz, 6H), 3.14 (q, J=6.5 Hz, 6H), 2.43 (dt, J=27.0, 6.7 Hz, 8H), 2.24 (t, J=7.2 Hz, 2H), 1.96 (p, J=7.5 Hz, 2H), 1.69-1.59 (m, 6H), 1.43 (d, J=5.8 Hz, 27H); MS (ESI): 1011.5 (M+H)+.

Step 5: To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate (1.84 g, 1.821 mmol) in DCM (40 mL) was added 2,2,2-trifluoroacetic acid (7.02 mL, 91 mmol). The reaction mixture was stirred at room temperature for overnight. Solvent was evaporated to give benzyl 5-((1,19-diamino-10-((3-((3-aminopropyl)amino)-3-oxopropoxy)methyl)-5,15-dioxo-8,12-dioxa-4,16-diazanonadecan-10-yl)amino)-5-oxopentanoate as a colorless oil. MS (ESI), 710.6 (M+H)⁺.

Step 6: To a solution of 4-sulfamoylbenzoic acid (1.466 g, 7.28 mmol) in DCM (40 mL) was added HATU (2.77 g, 7.28 mmol) followed by benzyl 5-((1,19-diamino-10-((3-((3-aminopropyl)amino)-3-oxopropoxy)methyl)-5,15-dioxo-8,12-dioxa-4,16-diazanonadecan-10-yl)amino)-5-oxopentanoate (1.293 g, 1.821 mmol) in DMF (4.0 mL). The mixture was stirred at room temperature for 5 hrs. Solvent was evaporated under reduced pressure to give a residue, which was purified by ISCO (40 g gold column) eluting with DCM to 50% MeOH in DCM to give benzyl 1,7,14-trioxo-12,12-bis((3-oxo-3-((3-(4-sulfamoylbenzamido)propyl)amino)-propoxy)methyl)-1-(4-sulfamoylphenyl)-10-oxa-2,6,13-triazaoctadecan-18-oate (0.36 g, 0.286 mmol, 16% yield). ¹H NMR (400 MHz, DMSO-d₆) δ 8.60 (t, J=5.6 Hz, 3H), 7.96-7.81 (m, 15H), 7.44 (s, 6H), 7.35-7.23 (m, 5H), 7.04 (s, 1H), 5.02 (s, 2H), 3.50 (t, J=6.9 Hz, 6H), 3.48 (s, 6H), 3.23 (q, J=6.6 Hz, 6H), 3.06 (q, J=6.6 Hz, 6H), 2.29 (t, J=7.4 Hz, 2H), 2.24 (t, J=6.5 Hz, 6H), 2.06 (t, J=7.4 Hz, 2H), 1.69-1.57 (m, 8H).

Step 7: To a round bottom flask flushed with Ar was added 10% Pd/C (80 mg, 0.286 mmol) and EtOAc (15 mL). A solution of benzyl 1,7,14-trioxo-12,12-bis((3-oxo-3-((3-(4-sulfamoylbenzamido)propyl)amino)propoxy)methyl)-1-(4-sulfamoylphenyl)-10-oxa-2,6,13-triazaoctadecan-18-oate (360 mg) in methanol (15 mL) was added followed by diethyl(methyl)silane (0.585 g, 5.72 mmol) dropwise. The mixture was stirred at room temperature for 3 hrs. LC-MS showed the reaction was complete. The reaction was diluted with EtOAc, and filtered through celite, washed with 20% MeOH in EtOAc, and concentrated under reduced pressure to give 1,7,14-trioxo-12,12-bis((3-oxo-3-((3-(4-sulfamoylbenzamido)propyl)-amino)propoxy)methyl)-1-(4-sulfamoylphenyl)-10-oxa-2,6,13-triazaoctadecan-18-oic acid (360 mg, 100% yield) as a white solid. ¹H NMR (400 MHz, DMSO-d₆) δ 8.60 (t, J=5.6 Hz, 3H), 7.94-7.81 (m, 15H), 7.44 (s, 6H), 7.04 (s, 1H), 3.50 (t, J=6.9 Hz, 6H), 3.48 (s, 6H), 3.23 (q, J=6.6 Hz, 6H), 3.06 (q, J=6.6 Hz, 6H), 2.24 (t, J=6.4 Hz, 6H), 2.14 (t, J=7.5 Hz, 2H), 2.05 (t, J=7.4 Hz, 2H), 1.66-1.57 (m, 8H); MS (ESI), 1170.4 (M+H)⁺.

### Example 9. Example Synthesis of Amidites for Mod030-Mod033

To a solution of lauryl alcohol (5.2 g, 28 mmol) in 60 mL dry DCM, under an atmosphere of argon, at room temperature was added DIPEA (18 g, 140 mmol) and stirred for 5 minutes. To this solution was added 2-cyanoethyl N,N-diisopropylchlorophosphoramidite (7.9 g, 33.5 mmol) dropwise and stirred for 4 hours. Solvent from the reaction mixture was evaporated under reduced pressure, diluted with 300 mL ethyl acetate, washed with sat. NaHCO₃ and dried over anhydrous sodium sulfate. Removal of solvent and column chromatography over silica gel (80 g regular silica, 0-30% ethyl acetate in hexane containing 5% triethyl amine) using ISCO afforded the product. Weight of product obtained: 3.8 g (35%). ¹H NMR (500 MHz; CDCl₃): δ 3.88-3.76 (m, 2H), 3.68-3.55 (m, 4H), 2.62 (t, 2H), 1.62-1.35 (m, 2H), 1.32-1.28 (m, 18H), 1.19-1.17 (m, 12H), 0.87 (t, 3H). ³¹P NMR (202.4 MHz; CDCl₃): δ 147.2 (s). Amidites for Mod031, Mod032 and Mod033 were prepared using the same procedure. These amidites were used as the last amidite in the synthesis cycle to prepare oligonucleotides comprising Mod030-Mod033.

### Example 10. Example Preparation of Acid for Mod024

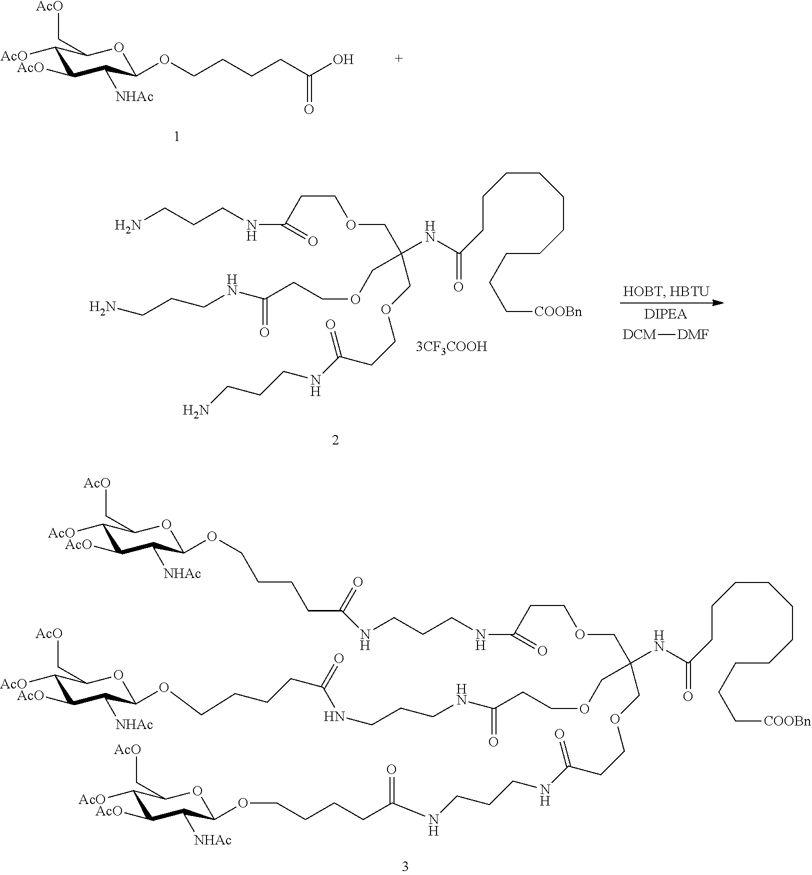

GlucNAc acid 1 (WO 2014/025805 A1) (1.88 g, 4.2 mmol) and HOBT (0.73 g, 5.4 mmol) was stirred in anhydrous DMF-DCM mixture (11+15 mL) under nitrogen at room temperature for 10 minutes. HBTU (2.05 g, 5.4 mmol) was added followed by DIPEA (2.17 g, 16.8 mmol) at 10° C. To this solution was added tri-amine salt 2 (WO 2014/025805 A1) (1.38 g, 1.2 mmol) and stirred overnight. Solvent was removed under vacuum and the residue was dissolved in ethyl acetate (200 mL). To this solution was added 100 ml of a mixture of sat. ammonium chloride, sat. sodium chloride, sat. sodium bicarbonate and water (1:1:1:1). The ethyl acetate layer was turbid initially. After thoroughly shaking the layers got separated. Aqueous layer was extracted with ethyl acetate (×2). Combined organic fractions were washed with brine and dried over anhydrous sodium sulfate. Solvent removal under reduced pressure afforded 490 mg of crude product. This product was purified by CC on an ISCO machine. The eluent was DCM-Methanol (0-20% methanol in DCM). Amount of product obtained was 1.26 g (50%). LC-MS (+ mode): 1768 (M-1GlucNAc), 1438 (M-2 GlucNAc), 1108 (M-3 GlucNAc), 1049 (M/2+1).

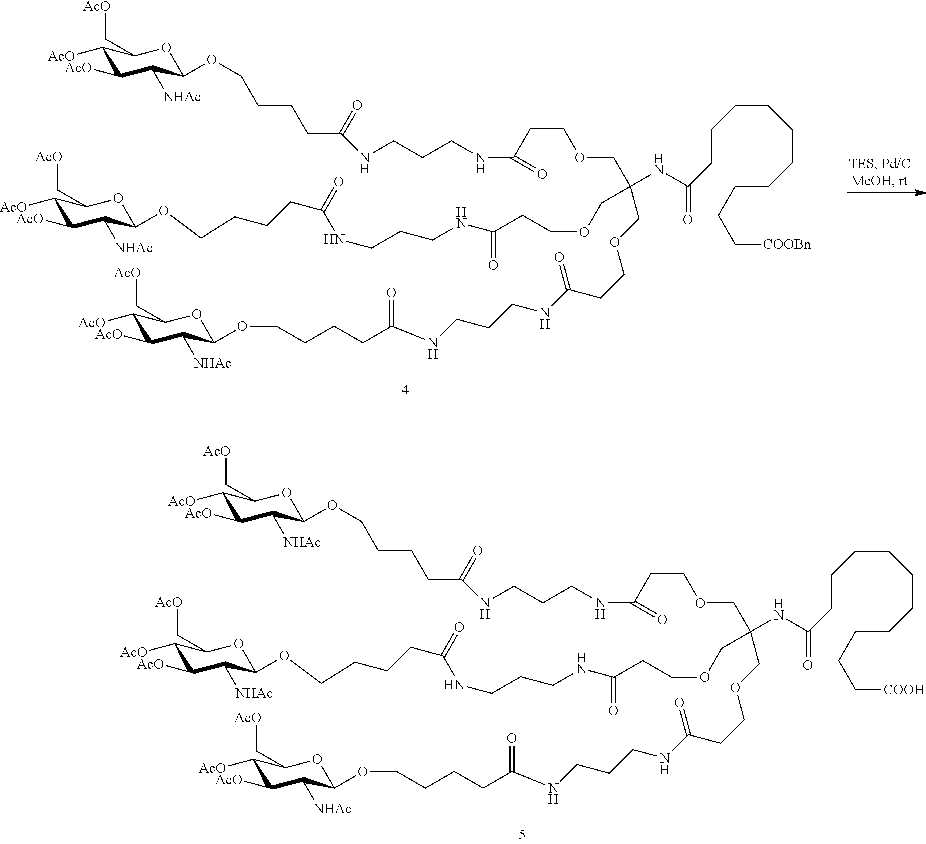

To a solution of benzyl ester 4 (0.25 g, 0.119 mmol) in 7 mL dry methanol, under an atmosphere of argon, 10% Pd/C (50 mg) was added followed by 1.5 mL (9.4 mmol) triethylsilane (TES) drop wise. A vigorous reaction set in and the RMV was stirred for 3 hours. LC-MS analysis of the product indicates completion of reaction. The RMV was filtered over celite and solvent was removed under vacuum. The crude product was triturated (×3) with ether-methanol (3:1) mixture and dried under vacuum. This product 5 was used for conjugation with oligonucleotide chains without further purification, and after conjugation the hydroxyl groups were deprotected, for example, during cleavage and/or deprotection of oligonucleotides to incorporate Mod024. If desired, a number of protocols can be utilized to deprotect the hydroxyl groups in 5 to provide the acid with deprotected hydroxyl groups. ¹H NMR (500 MHz, DMSO-D6): δ 7.90 (3H, d, J=N0 Hz), 7.80 (t, 3H), 7.70 (t, 3H), 5.03 (t, 3H), 4.77 (t, 3H), 4.54 (3H, d, J=10 Hz), 4.14 (3H, dd, J₁=9 Hz, J₂=5 Hz), 3.97-3.93 (m, 3H), 3.79-3.74 (m, 3H), 3.69-3.61 (m, 6H), 3.51-3.47 (m, 3H), 3.40-3.35 (m, 3H), 3.31 (d, 3H, J=9 Hz), 2.98 (m, 12H), 2.23 (t, 3H), 2.13 (t, 3H), 2.01-1.99 (m, 3H), 1.97 (s, 9H), 1.92 (s, 9H), 1.86 (s, 9H), 1.71 (s, 9H), 1.49-1.32 (m, 22H), 1.18 (br s, 12H). Mod026 were incorporated using similar strategies.

### Example 11. Example Procedure for Conjugation—Preparing Oligonucleotide Chains with Amino Groups

As appreciated by a person having ordinary skill in the art, various technologies, e.g., linkers, methods, functional groups, etc. can be utilized to prepare provided oligonucleotides in accordance with the present disclosure, including those comprising lipid moieties and/or targeting components. Below are example procedures for preparing oligonucleotides with amino groups for incorporating various moieties, e.g., lipid moieties, targeting components, etc. A person of ordinary skill in the art appreciates that various technologies can be used to conjugate lipids with other types of biologically active agents, e.g., small molecules, peptides, proteins, etc., including methods, reagents, etc. widely known and used in the art, in accordance with the present disclosures.

“On Support” Conjugation Strategy

Preparation of 5′-amino-modified oligonucleotides for “on support” conjugation was carried out using MMT-amino C6 CE phosphoramidite (ChemGenes Corporation catalog No. CLP-1563 or Glen Research catalog No. 10-1906), which was added as the last phosphoramidite and coupled to 5′-OH of the oligonucleotide chain on solid support using oligonucleotide synthesis chemistry. After coupling, the newly formed linkage was optionally oxidized to provide a phosphodiester linkage if desired using, for example, tert-butyl hydroperoxide (e.g., 1.1 M in 20:80 decane/dichloromethane), 12 (e.g., in pyridine/water, THF/pyridine/water, etc.), etc., depending on the oligonucleotide synthesis chemistry. When a phosphorothioate linkage was desired, PolyOrg Sulfa (e.g., 0.1 M in acetonitrile) or DDTT (e.g., 0.1 M in pyridine) was used for sulfurization. The MMT protecting group was then removed while the oligonucleotide was on support with deblocking reagent (e.g., 3% trichloroacetic acid in dichloromethane, 3% dichloroacetic acid in toluene, etc.) until the yellow color was no longer observed. Various compounds, e.g., fatty acids, sugar acids, etc. were then coupled, and optionally followed by cleavage from the support, deprotection and/or purification.

“In Solution” Conjugation Strategy

Preparation of 5′-amino-modified oligonucleotides for “in solution” conjugation strategy was carried out using TFA-amino C6 CED phosphoramidite (ChemGenes Corporation catalog No. CLP-1553 or Glen Research catalog No. 10-1916), which was added as the last phosphoramidite and coupled to 5′-OH of the oligonucleotide chain on solid support using oligonucleotide synthesis chemistry. After coupling, the newly formed linkage was optionally oxidized to provide a phosphodiester linkage if desired using, for example, tert-butyl hydroperoxide (e.g., 1.1 M in 20:80 decane/dichloromethane), 12 (e.g., in pyridine/water, THF/pyridine/water, etc.), etc., depending on the oligonucleotide synthesis chemistry. When a phosphorothioate linkage was desired, PolyOrg Sulfa (e.g., 0.1 M in acetonitrile) or DDTT (e.g., 0.1 M in pyridine) was used for sulfurization. The amine-modified oligonucleotides were then cleaved from the support, deprotected and purified to provide products with free amino groups for conjugation. Usually the TFA group was removed during cleavage and deprotection of the oligonucleotides. The oligonucleotides were then utilized for conjugation

### Example 12. Example Procedure for Conjugation on Solid Support

In some embodiments, lipid conjugation to biologically active agents can be performed using solid support. As appreciated by a person having ordinary skill in the art, a number of widely known and practiced technologies, e.g., reagents, methods, etc., can be utilized to prepare provided oligonucleotide compositions, including those comprising lipid moieties, in accordance with the present disclosure. Two example schemes are provided in the present and following examples for illustration of conjugation of lipids, targeting components, etc. to oligonucleotides. In some embodiments, R^{LD}—COOH is a fatty acid as described herein (prepared and/or commercially available) to provide R^{LD} as illustrated in provided oligonucleotides, e.g., certain example oligonucleotides in Table 4. In some embodiments, R^{LD}—COOH is an acid comprising targeting component (prepared and/or commercially available) as described herein to provide R^{LD} as illustrated in provided oligonucleotides, e.g., certain example oligonucleotides in Table 4.

Example Procedure for Conjugation on Solid Support:

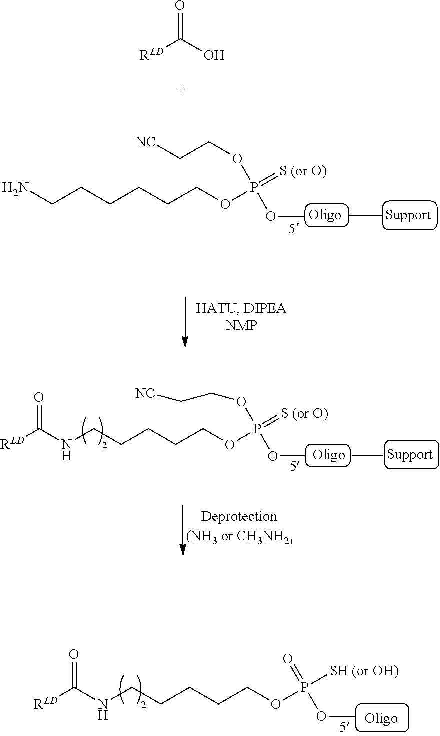

In an example procedure, a mixture of a lipid acid (1 μmol, 1 eq.), HATU (0.9 eq), diisopropylethylamine (10 eq) and NMP (500 μl) was shaken well at room temperature for 10 minutes, in a 3 mL plastic vial. This activated acid was pipetted into a plastic vial containing oligonucleotides (e.g., see example above) on solid support (0.09 μmol, 0.9 eq). The contents of the vial was thoroughly mixed and shaken well for 12 hours. After this time the supernatant NMP was removed carefully. The solid support was washed with acetonitrile (1 mL×3) and dried in a speed vac. A 1:1 mixture (1 mL) of ammonium hydroxide and methyl amine (AMA) was added and heated at 35° C. for 1 hour with intermittent shaking. After 1 hour, the CPG was transferred into a small filtration cartridge, filtered, washed with DMSO (500 μl×2) and washed with water (1 mL×3). Filtrate and washings were combined and diluted to 10 mL using water. This solution was cooled to 0° C. and neutralized with glacial acetic acid until pH of the solution reached 7.5. (Alternatively the dried solid support can be treated with 35% NH₄OH at 60° C. for 12 hours, cooled, filtered and neutralized with glacial acetic acid. For oligos containing fluoro group at 2′ position, a mixture of 35% ammonium hydroxide and ethanol (3:1) was used with temperature not exceeding 40° C.). Crude product was analyzed by UV spectrometer, reverse phase HPLC and LC-MS. Purification of the crude product was done by RP-HPLC. After HPLC purification each fraction was analyzed by RP-HPLC and LC-MS. Pure fractions were combined and solvent was removed under vacuum (speed vac). Residue was dissolved in water and desalted (Triethyl ammonium ion was replaced with sodium ion) on a C-18 cartridge. Solvent was removed on a speed vaac and the residue was filtered through a centrifugal filter (Amicon Ultra-15 by Millipore), lyophilized and analyzed.

For example, for synthesis of WV-2578, a mixture of lauric acid (11.01 mg, 0.0549 mmol), HATU (19 mg, 0.050 mmol) and diisopropylethyl amine (18 μL, 0.1 mmol) was dissolved in 500 μL of dry NMP and shaken well for five minutes. This activated acid was pipetted into a plastic vial containing oligonucleotides on solid support (70.5 mg, 0.005 mmol). The contents of the vial was thoroughly mixed and shaken well for 12 hours. After this time supernatant NMP was removed carefully. The solid support was washed with acetonitrile (1 mL×3) and dried in a speed vac. A 1:1 mixture (1 mL) of ammonium hydroxide and methyl amine (AMA) was added and heated at 35° C. for 1 hour with intermittent shaking. After 1 hour, the CPG was transferred into a small filtration cartridge, filtered, washed with DMSO (500 μL×2) and washed with water (1 mL×3). Filtrate and washings were combined and diluted to 10 mL using water. This solution was cooled to 0° C. and neutralized with glacial acetic acid until pH of the solution reached 7.5. Purification of the crude product was done by RP-HPLC. After HPLC purification each fraction was analyzed by RP-HPLC and LC-MS. Pure fractions were combined and solvent was removed under vacuum (speed vac). Residue was dissolved in water and desalted (triethyl ammonium ion was replaced with sodium ion) on a C-18 cartridge. Solvent was removed on a speed vac and the residue was filtered through a centrifugal filter (Amicon Ultra-15 by Millipore), lyophilized and analyzed. Average mass of WV2578 calculated: 7355, found (deconvoluted mass):7358. Additional examples include:

| | CPG | |
|---|---|---|
| EXP* | (5 μmol) | Acid (55 μmol) |
| 1 | 70.5 | Lauric acid (MW = 200.32) 11.01 mg |
| 2 | 70.5 | Myristic Acid (MW = 228.38) 12.56 mg |
| 3 | 70.5 | Palmitic acid (MW = 256.26) 14.1 mg |
| 4 | 70.5 | Stearic acid (MW = 284.27) 15.63 mg |
| 5 | 70.5 | Oleic acid (MW = 282.47) 15.53 g |
| 6 | 70.5 | Linolenic acid (MW = 280.45) 15.4 mg |
| 7 | 70.5 | α-Linolenic acid (MW = 278.44) 15.3 mg |
| 8 | 70.5 | γ-Linolenic acid (MW = 278.44) 15.3 mg |
| 9 | 70.5 | cis-DHA (MW = 328.24) 18.05 mg |
| 10 | 70.5 | Turbinaric acid (MW = 400.36) 22 mg |

*HATU (50 μmol, MW = 379.24, 19 mg),
DIPEA (MW = 129, d = 0.726, 100 μmol, 18 μL),
NMP (500 μL).
Example products include (Total ODs and Amount of lipid conjugates after purification; some may be described in previous examples):

| | Conjugated | Total | Amount | Amount |
|---|---|---|---|---|
| Oligonucleotide | Acid | ODs | (μmol) | (mg) |
| WV2578 | Lauric Acid | 287 | 1.40 | 9.79 |
| WV2579 | Myristic Acid | 331 | 1.62 | 11.29 |
| WV2580 | Palmitic Acid | 268 | 1.31 | 9.14 |
| WV2581 | Stearic Acid | 265 | 1.30 | 9.04 |
| WV2582 | Oleic Acid | 262 | 1.28 | 8.94 |
| WV2583 | Linoleic Acid | 120 | 0.59 | 4.09 |
| WV2584 | α-Linolenic Acid | 285 | 1.39 | 9.72 |
| WV2585 | γ-Linolenic Acid | 297 | 1.45 | 10.13 |
| WV2586 | cis-DHA | 274 | 1.34 | 9.35 |
| WV2587 | Turbinaric acid | 186 | 0.91 | 6.35 |
| WV2588 | Dilinoleyl* | 345 | 1.69 | 11.77 |

*Synthesized on a solid support; last cycle using 2-cyanoethyl ((6Z,9Z,28Z,31Z)-heptatriaconta-6,9,28,31-tetraen-19-yl) diisopropylphosphoramidite.

### Example 13. Example Procedure for Conjugation in Solution

In some embodiments, lipid conjugation with biologically active agents can be performed in solution. In some embodiments, provided oligonucleotides comprising lipid moieties were prepared in solution phase.

Example Procedure for Conjugation in Liquid Phase:

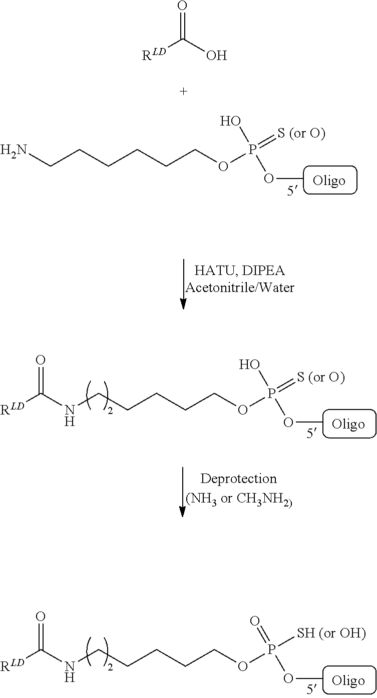

In an example procedure, a mixture of the lipid acid (1 eq.), HATU (1 eq.) and DIPEA (10 eq.) was mixed well in dry AcCN (10 mL) and kept for 10 minutes. This activated acid was added to the oligonucleotide (5 μmol) in water (5 mL) and mixed well on a vortex. This reaction was shaken for 1 hour. After 1 hour completion of the reaction was checked by LC-MS (usually the reaction is complete in 1 hour; if not, more acid-HATU complex can be added to drive the reaction to completion). Acetonitrile and water was removed under vacuum on a speed vac. The solid obtained was treated with 35% ammonium hydroxide (15 mL) and shaken at 60° C. for 12 hours; for 2′ fluoro oligonucleotides a 3:1 mixture of 35% ammonium hydroxide and ethanol was used for deprotection). After 12 hours solvent was removed under vacuum and diluted with water (15 mL), analyzed by LC-MS and RP-HPLC. Crude product was then purified by RP-HPLC and desalted.

For example, for synthesis of WV-3546, turbinaric acid (7 mg, 0.0174 mmol), HATU (6.27 mg, 0.0165 mmol) and DIPEA (22.2 mg, 0.172 mmol) was mixed well in dry AcCN (10 mL) and kept for 5 minutes in a 40 mL plastic vial. This activated acid was added to oligonucleotides in 3.77 mL water (80 mg, 0.0117 mmol) and mixed well on a vortex. This reaction was shaken for 2 hours. After 2 hours completion of the reaction was checked by LC-MS (reaction was complete). Acetonitrile and water was removed under vacuum on a speed vac. The solid obtained was treated with ammonia: ethanol mixture (3:1, 15 mL) and shaken at 40° C. for 12 hours. After 12 hours solvent was removed under vacuum and diluted with water (˜15 mL) and analyzed by LC-MS. Crude product was purified by RP-HPLC (50 mM triethyl ammonium acetate in water-acetonitrile system (0-70% acetonitrile in 45 minutes), X Bridge preparative C8 (19×250 mm column)). Average mass of WV3546 calculated: 7295. Mass found (deconvoluted mass): 7295.

### Example 14. Example Synthesis of MMT-C6-Amino DPSE-L Amidite

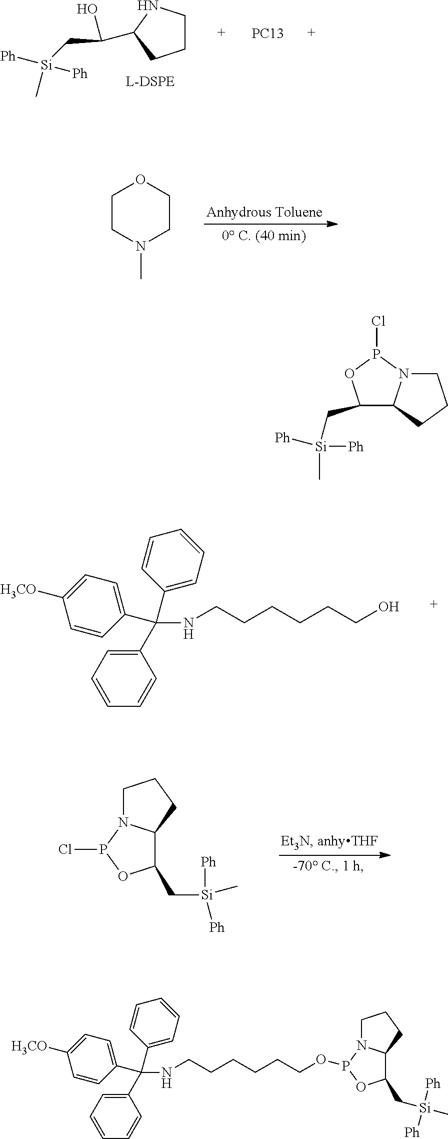

Preparation of chlorooxazaphospholidine: L-DPSE (37.1 g, 119 mmol) was dried by azeotropic evaporation with anhydrous toluene (150 mL) at 35° C. in a rotaevaporator and left in high vacuum for overnight. Then a solution of this dried L-DPSE (37.1 g) and 4-methylmorpholine (26.4 mL, 24.31 g, 240 mmol) dissolved in anhydrous toluene (150 mL) was added to an ice-cold solution of trichlorophosphine (16.51 g, 10.49 mL, 120 mmol) in anhydrous toluene (110 mL) placed in three neck round bottomed flask through cannula under Argon (start Temp: 0.6° C., Max: temp 14° C., 25 min addition) and the reaction mixture was stirred at 0° C. for 40 min. After that the precipitated white solid was filtered by vacuum under argon using spacial filter tube (Chemglass: Filter Tube, 24/40 Inner Joints, 80 mm OD Medium Frit, Airfree, Schlenk). The solvent was removed by rotaevaporator under argon at low temperature (25° C.) followed by dried under vacuum overnight (˜15 h) and the oily chlorooxazaphospholidine obtained was used for the next step.

MMT-C6-amino DPSE-L amidite: 6-(monomethoxytritylamino)hexan-1-ol (7.0 g, 17.97 mmol) was first dried by azeotropic evaporation by anhydrous toluene (50 ml) and dried under vacuum for overnight. Then the dried 6-(monomethoxytritylamino)hexan-1-ol was dissolved in anhydrous THE (80 mL) and added triethylamine (9.0 g, 90 mmol) and then the reaction solution was cooled to −70° C. To this cooled solution was added chlorooxazaphospholidine (6.76 g, 17.97 mmol) dissolved in anhydrous THF (50 mL) over 10 min. After the reaction mixture slowly warmed to room temperature (˜1 h), TLC indicated complete conversion of starting material. Then the reaction mixture was filtered carefully under vacuum/argon using the fitted filtration tube to remove precipitated solid, and washed with THE (80 mL). The solution was evaporated at 25° C. and the resulting oily residue was dissolved in Hexane-CH₂Cl₂ mixture with 5% TEA and purified using ISCO Combi-Flash system 220 g silica column (which was pre-de-activated with 3 CV MeOH, then equilibrated with ethyl acetate (5% TEA) 3 CV), with Hexane-EtOAc mixture (5% TEA). Pure fractions were collected and concentrated, dried overnight to afford MMT-C6-amino DPSE-L amidite as a colorless oily liquid. Yield: 8.0 g (62%). MS: calculated: 728.38; found by LCMS analysis at +Ve ion mode m/z: 729.54 (M⁺ ion), 747.50 (M⁺+18, H2O). ¹H-NMR (500 MHz, CDCl₃): δ 7.58-7.43 (m, 8H), 7.41-7.31 (m, 6H), 7.31-7.23 (m, 6H), 7.17 (t, J=7.2 Hz, 2H), 6.81 (d, J=8.7 Hz, 2H), 4.82 (dt, J=8.7, 5.7 Hz, 1H), 3.78 (s, 3H), 3.77-3.73 (m, 1H), 3.54 (qt, J=11.0, 5.2 Hz, 2H), 2.54 (q, J=7.2 Hz, 3H), 2.11 (t, J=7.0 Hz, 2H), 1.64-1.57 (m, 4H), 1.51-1.35 (m, 6H), 1.26 (q, J=9.9, 8.0 Hz, 2H), 1.04 (t, J=7.1 Hz, 2H), 0.67 (s, 3H). ¹³C NMR (500 MHz, CDCl₃) δ 157.87, 146.73, 146.67, 138.63, 136.89, 136.43, 134.71, 134.57, 134.48, 129.88, 129.46, 129.42, 128.66, 128.05, 127.96, 127.87, 127.81, 126.17, 113.13, 78.14, 78.07, 77.48, 77.43, 77.22, 76.97, 70.45, 68.03, 68.01, 63.50, 63.40, 55.22, 47.46, 47.17, 46.40, 43.69, 34.79, 31.34, 31.07, 27.19, 27.09, 26.04, 25.98, 17.60, 11.78, −3.17. ³¹P-NMR (500 MHz, CDCl₃): δ 154.27 (92.18%), 157.68 (3.56%), 146.35 (4.26%).

### Example 15. Example Preparation of WV-4107

Oligonucleotides were prepared using conditions for WV-3473 with all protecting groups and auxiliaries on and remained on solid support (if cleaved and deprotected, would provide WV-3473) using provided oligonucleotide technologies. In an example procedure, DPSE chemistry and GE Primer Support 5G (2.1 g), and the following cycles were used:

| | | | volume | waiting |
|---|---|---|---|---|
| step | operation | reagents and solvent | per cycle | time |
| 1 | detritylation | 3% DCA in toluene | ~150 mL | ~6 min |
| 2 | coupling | 0.175M monomer in MeCN or | 21 mL | 8 min |
| | | 20% isobutyronitrile in MeCN + 0.6M | | |
| | | CMIMT in MeCN | | |
| 3 | capping | 20% Ac₂O, 30% 2,6-lutidine in | 23 mL | 1.5 min |
| | | MeCN + 20% MeIm in MeCN | | |
| 4 | oxidation or | 1.1M TBHP in DCM-decane | 44 mL or | 2 min or 6 min |
| | sulfurization | or 0.1M POS in MeCN | 39 mL | |

After the last cycle, a portion of the oligonucleotides can be cleaved and deprotected for QC or other purposes. In an example procedure the oligonucleotides on support were washed with 6 column volumes of 20% diethylamine in acetonitrile for 15 min followed by an acetonitrile wash. The support was dried and then incubated in 1 M triethylamine hydrofluoride in 3:1 dimethylformamide/water for 1-1.5 h at 50° C. The sample was filtered and washed with acetonitrile and dried. The support was then incubated overnight at 40° C. in 3:1 ammonium hydroxide/ethanol.

For preparation of WV-4107, after the last cycle the DMT protecting group was removed using 3% dichloroacetic acid in toluene. During the coupling step, MMT-C6-amino DPSE-L amidite (0.175 M in isobutyronitrile) and CMIMT activator (0.6 M in acetonitrile) were added with a contact time of 8 min. The percent volume of activator was 55%. Capping was performed with 20% 1-methylimidazole in acetonitrile and 20/30/50 acetic anhydride/2,6-lutidine/acetonitrile. Sulfurization was performed using 0.1 M PolyOrg Sulfa in acetonitrile.

The MMT protecting group was then removed while the oligonucleotide was on support with deblocking reagent (3% dichloroacetic acid in toluene) until the yellow color was no longer observed, providing WV-4191. Stearic acid was then coupled to the amine using described procedure above. The oligonucleotides on support were washed with 20% diethylamine in acetonitrile for 30 min at room temperature followed by an acetonitrile wash. The support was dried and then incubated in 1 M triethylamine hydrofluoride in 3:1 dimethylformamide/water for 1-1.5 h at 50° C. The sample was filtered and washed with acetonitrile and dried. The support was then incubated overnight at 40° C. in 3:1 ammonium hydroxide/ethanol. The crude product was further purified using RP-HPLC to provide WV-4107.

### Example 16. Example Preparation of Oligonucleotides with Mod021

Oligonucleotide was synthesized at a scale of 10 μmol using standard cyanoethyl phosphoramidite chemistry and was left on support with protecting groups using cycle conditions for WV-942 (if cleaved and deprotected, would provide WV-942). The DMT protecting group was removed using 3% trichloroacetic acid in dichloromethane. The lipid amidite was then added to the 5′ end of the oligonucleotide on the synthesizer. During the coupling step, equal volumes of lipid amidite (e.g., 0.1 M in isobutyronitrile) and 5-ethylthio tetrazole (e.g., 0.5 M in acetonitrile) were added with a contact time of, e.g., 5 min. The coupling step was optionally repeated a second time. Sulfurization was performed using 0.1 M DDTT in pyridine. The oligonucleotide was cleaved and deprotected using AMA condition (ammonium hydroxide/40% aqueous methylamine 1:1 v/v) to provide WV-2588.

### Example 17. Example Preparation of Oligonucleotides with Mod030, Mod031, Mod032 and Mod033

Oligonucleotides were synthesized using cyanoethyl phosphoramidite chemistry as for WV-2735 and were left on support with the protecting group on (if cleaved and deprotected, would provide WV-2735). The 5′-DMT protecting group was removed using 3% trichloroacetic acid in dichloromethane. The lipid amidites were then added to the 5′ end of the oligonucleotide on the synthesizer. During the coupling step, equal volumes of lipid amidite (0.1M in isobutyronitrile or dichloromethane) and 5-ethylthio tetrazole (0.5M in acetonitrile) were added with a contact time of 10 min. The coupling step was repeated again. Oxidation was performed using 0.02 M I₂ in THF/pyridine/water. The oligonucleotides were de-protected with 20% diethylamine in acetonitrile wash followed by an acetonitrile wash. The oligonucleotides were cleaved from the support and further de-protected in ammonium hydroxide at 50° C. overnight.

Product oligonucleotides were characterized in various chemical analyses, e.g., UV, HPLC-MS, etc., (for example MS data, see Table 6) and biological assays, e.g., those described herein. Following similar procedures and/or using widely known and practiced technologies in the art, other example provided oligonucleotides were or can be readily prepared and characterized in accordance with the present disclosure.

### EQUIVALENTS

Having described some illustrative embodiments of the disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1.-5. (canceled)

6. A chirally controlled oligonucleotide composition comprising a plurality of oligonucleotides, wherein oligonucleotides of the plurality share:
1) a common base sequence;
2) a common pattern of backbone linkages; and
3) a common pattern of backbone phosphorus modifications;
wherein:
oligonucleotides of the plurality each comprise two or more consecutive 2′-F modified sugars;
50% or more of sugar moieties in each oligonucleotide of the plurality are 2′-F modified sugars;
oligonucleotides of the plurality share the same stereochemistry at five or more chiral internucleotidic linkages;
one or more-oligonucleotides of the plurality are individually conjugated to a lipid, wherein the lipid comprises an optionally substituted C₁₀-C₆₀ linear, saturated or partially unsaturated aliphatic chain.

7.-9. (canceled)

10. The composition of claim 6, wherein the plurality of oligonucleotides share the same stereochemistry at each chiral internucleotidic linkage.

11. (canceled)

12. The composition of claim 6, wherein the plurality of oligonucleotides share a common pattern of sugar modification, which comprises 5 or more consecutive 2′-F.

13.-15. (canceled)

16. A method of delivering an oligonucleotide to a muscle cell or tissue in a human subject, comprising:
(a) providing a composition of claim 6; and
(b) administering the composition to the human subject such that the oligonucleotide is delivered to a muscle cell or tissue in the subject.

17. A method of modulating the level of a transcript or gene product of a gene in a cell, the method comprising the step of contacting the cell with a composition claim 6, wherein the biologically active agent is capable of modulating the level of the transcript or gene product.

18. A method for treating a sign and/or symptom of a disease, disorder, or condition in a subject selected from cancer, a proliferative disease, disorder, or condition, a metabolic disease, disorder, or condition, an inflammatory disease, disorder, or condition, and a viral infection by providing a composition of claim 6 and administering the composition to the subject.

19.-21. (canceled)

22. The composition of claim 6, wherein each oligonucleotide of the plurality is at least 15 nucleotides in length.

23. The composition of claim 6, wherein each oligonucleotide of the plurality comprises 10 or more modified internucleotidic linkages.

24. The composition of claim 23, wherein each modified internucleotidic linkage is independently a phosphorothioate linkage.

25. The composition of claim 24, wherein at least about 80% phosphorothioate internucleotidic linkages of each oligonucleotide of the plurality are of the Sp configuration.

26. The composition of claim 24, wherein at least about 90% phosphorothioate internucleotidic linkages of each oligonucleotide of the plurality are of the Sp configuration.

27. The composition of claim 6, wherein oligonucleotides of the plurality comprise one or more natural phosphate linkages.

28. The composition of claim 6, wherein oligonucleotides of the plurality comprise one or more 2′-OR¹ modified sugars, wherein R¹ is optionally substituted C₁₋₆ aliphatic.

29. The composition of claim 28, wherein oligonucleotides of the plurality comprise one or more 2′-OMe modified sugars.

30. The composition of claim 6, wherein oligonucleotides of the plurality are identical.

31. A pharmaceutical composition comprising the composition of claim 6 and at least one pharmaceutically acceptable inactivate ingredient selected from pharmaceutically acceptable diluents, pharmaceutically acceptable excipients, and pharmaceutically acceptable carriers.

32. The pharmaceutical composition of claim 31, wherein each oligonucleotide of the plurality is independently and optionally a pharmaceutically acceptable salt.
